(19) United States
(12) Patent Application Publication
(10) Pub. No.: US 2026/0007758 A1
(43) Pub. Date: Jan. 8, 2026

(54) SOMATOSTATIN SUBTYPE-2 RECEPTOR (SST2R) NON-PEPTIDE DRUG CONJUGATES AND USES THEREOF

(71) Applicant: Crinetics Pharmaceuticals, Inc., San Diego, CA (US)

(72) Inventors: Jian ZHAO, San Diego, CA (US); Mi CHEN, San Diego, CA (US); Yunfei ZHU, San Diego, CA (US); Jeffrey SCHKERYANTZ, Harvard, MA (US)

(21) Appl. No.: 19/232,304

(22) Filed: Jun. 9, 2025

Related U.S. Application Data
(60) Provisional application No. 63/658,766, filed on Jun. 11, 2024.
(60) Provisional application No. 63/787,101, filed on Apr. 11, 2025.

(51) Int. Cl. A61K 47/65; A61P 35/00

(52) U.S. Cl. CPC A61K 47/65; A61P 35/00

(57) ABSTRACT

Described herein are somatostatin subtype-2 receptor (SST2R) non-peptide drug conjugates (NDC) that target tumor cells expressing SST2R and their use in the treatment and/or diagnosis of cancer.

### CROSS-REFERENCE TO RELATED APPLICATIONS

This application claims the benefit of U.S. Provisional Patent Application No. 63/658,766, filed Jun. 11, 2024; and U.S. Provisional Patent Application No. 63/787,101, filed Apr. 11, 2025; each of which is incorporated herein in its entirety by reference.

### FIELD OF THE INVENTION

Described herein are somatostatin subtype-2 receptor (SST2R) non-peptide drug conjugates (NDC) and methods of using such conjugates as cancer therapeutics, diagnostics, or both.

### BACKGROUND OF THE INVENTION

Neoplasms are abnormal growth of cells and cause enormous medical burdens, including morbidity and mortality, in humans. Neoplasms include benign or noncancerous neoplasms which do not display malignant features and are generally unlikely to become dangerous (e.g., adenomas); malignant neoplasms display features such as genetic mutations, loss of normal function, rapid division, ability to metastasize (invade) to other tissues, and exhibit neoplasms of uncertain or unknown behavior. Malignant neoplasms (i.e., cancerous solid tumors) are the leading cause of death in industrialized countries. Noncancerous neoplasms, including benign adenomas, can also cause significant morbidity and mortality. Although standard treatments can achieve significant effects in tumor growth inhibition and even tumor elimination, the applied drugs exhibit only minor selectivity for the malignant tissue over healthy tissue and their severe side effects limit their efficacy and use. Specific targeting of neoplastic cells without affecting healthy tissue is a major desire for effective solid tumor therapy. Non-peptide SST2R ligands conjugated to suitable drug cargos or payloads represent a novel class of non-peptide drug conjugates (NDC) for selective cancer therapeutics or diagnostics.

### SUMMARY OF THE INVENTION

Described herein are SST2R modulators that target the delivery of payloads to tumors that express the SSTR2, and their use in the treatment of tumors. The present disclosure provides an alternative and improved method for the treatment of tumors. In some embodiments, the NDCs disclosed herein provide an improved method for targeting SST2R-expressing tumor cells over traditional therapies, which have narrow therapeutic indexes.

In one aspect, described herein is a compound that has the structure of Formula (I), or a pharmaceutically acceptable salt thereof:

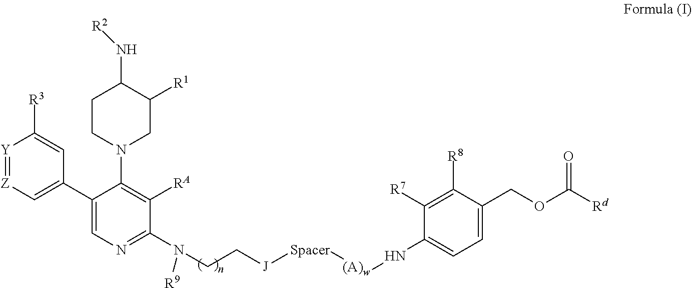

wherein:
J is a bond or NR⁶;
n is an integer selected from 1-5;
R^{A} is

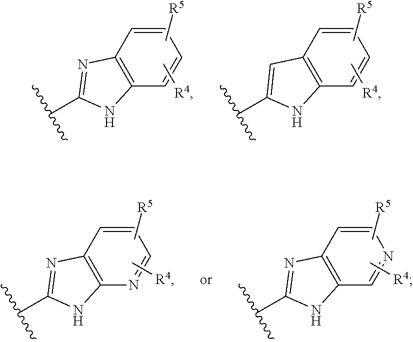

Y is N or CH;
Z is N or CR¹⁰;
R¹ is H;
R² is H or C₁-C₆ alkyl; or
R¹ and R², together with the N and C to which they are attached, combine to form a heterocycloalkyl;
R³ is C₁-C₆ alkyl or C₁-C₆ alkoxy;
R⁴ is H, halogen, CN, or C₁-C₆ alkoxy;
R⁵ is H, halogen, or CN;
R⁶ is H or C₁-C₆ alkyl;
R⁹ is H or C₁-C₆ alkyl;
R¹⁰ is H or halogen;
Spacer is a spacer group;
each A is independently selected from natural or unnatural amino acids, wherein any free amine of an amino acid is optionally independently substituted with CH₃;
w is an integer selected from 1-6;
R⁷ is H, C₁-C₆ haloalkyl, OR^{7a},

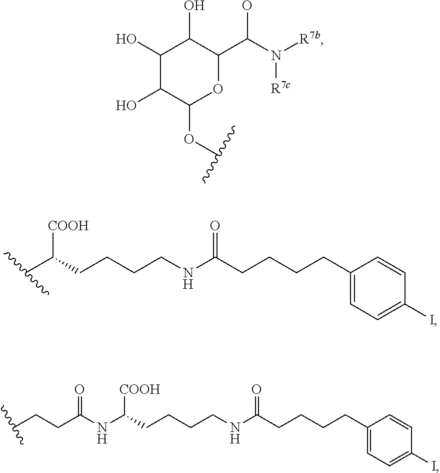

or C₁-C₆ alkyl optionally substituted with COOH;
R^{7a} is H, or C₁-C₆ alkyl optionally substituted with amino, COOH, or OH.
R^{7b} is H, C₁-C₂₀ alkyl optionally substituted with OR^{7b1}, COOR^{7b1}, NR^{7b1}₂, or SO₃R^{7b1}; (CH₂)₂NHC(═O)—R^{7b2}.

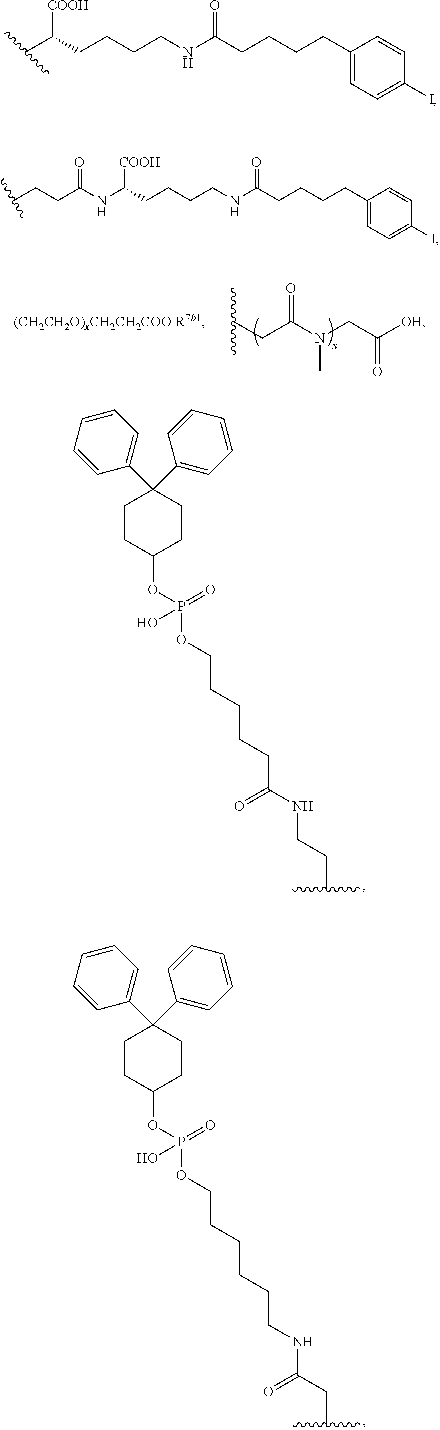

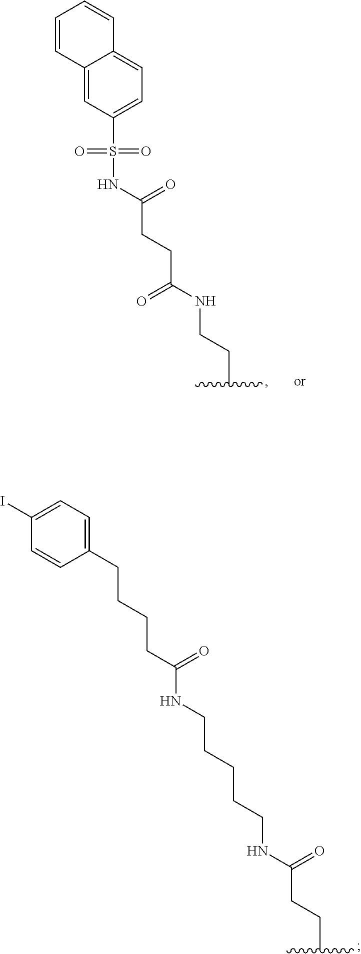 [SMILES: *CCNC(=O)CCC(=O)NS(=O)(=O)c1ccc2ccccc2c1 ; *CCC(=O)NCCCCCNC(=O)CCCCc1ccc(I)cc1]

each R^{7b1} is independently selected from H and C₁-C₆alkyl;

R^{7b2} is (CH₂CH₂O)ₓCH₂CH₂COOH, or (CH₂)_{y}COOH;
R^{7c} is H or C₁-C₆ alkyl; or
R^{7b} and R^{7c}, together with the nitrogen to which they are attached, combine to form a heterocycloalkyl group optionally substituted with amino, COOH, or C₁-C₆ alkyl that is optionally substituted with OH or COOH:

R⁸ is H, C₁-C₆ haloalkyl, COOH, C(═O)NH—R^{8a},

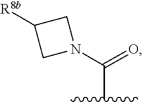

or C(═O)NH(CH₂)₂NHC(═O)—R^{8c};
R^{8a} is C₁-C₂₀ alkyl substituted with COOH or SO₃H, or C₁-C₆ alkoxy optionally substituted with amino;
R^{8b} is COOH or amino;
R^{8c} is (CH₂CH₂O)ₓCH₂CH₂COOH or (CH₂)_{y}COOH;
each x is independently an integer selected from 1-10;
each y is independently an integer selected from 1-20;
R^{d} is the residue of a bioactive molecule of the formula R^{d}—H;
wherein when R⁷ is H, then R⁸ is C₁-C₆ haloalkyl, COOH, C(═O)NH—R^{8a},

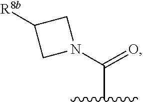

or C(═O)NH(CH₂)₂NHC(═O)—R^{8c}; and
wherein when R⁸ is H, then R⁷ is C₁-C₆ haloalkyl, OR^{7a},

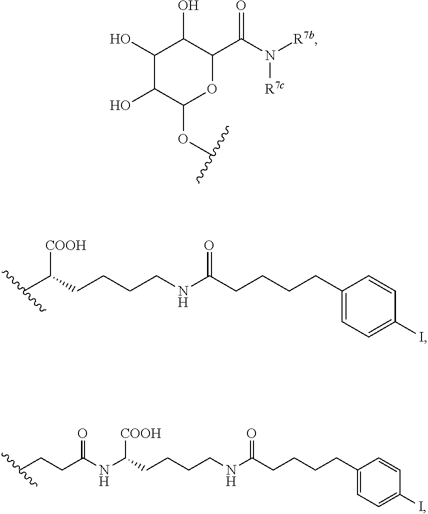

or C₁-C₆ alkyl optionally substituted with COOH.

In some embodiments, R⁷ is H, C₁-C₃ haloalkyl, OR^{7a},

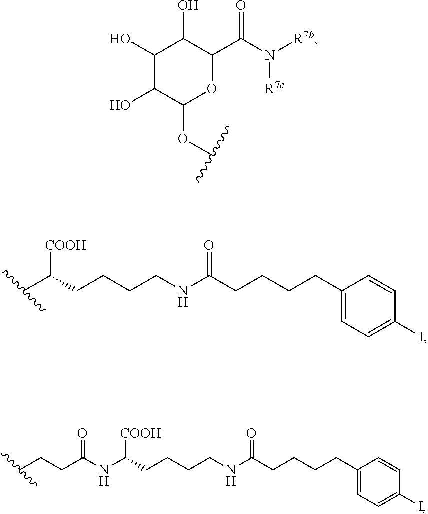

or C₁-C₃ alkyl optionally substituted with COOH;
R^{7a} is H, or C₁-C₃ alkyl optionally substituted with amino, COOH or OH;
H, C₁-C₁₄ alkyl optionally substituted with OR^{7b1}, COOR^{7b1}, NR^{7b1}₂, or SO₃R^{7b1}; (CH₂)₂NHC(═O)—R^{7b2},

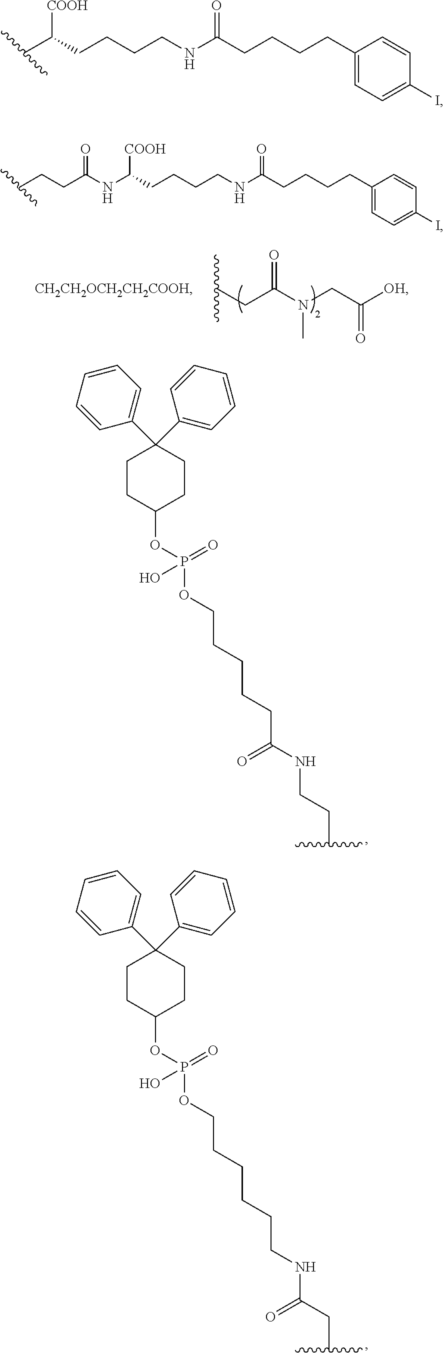

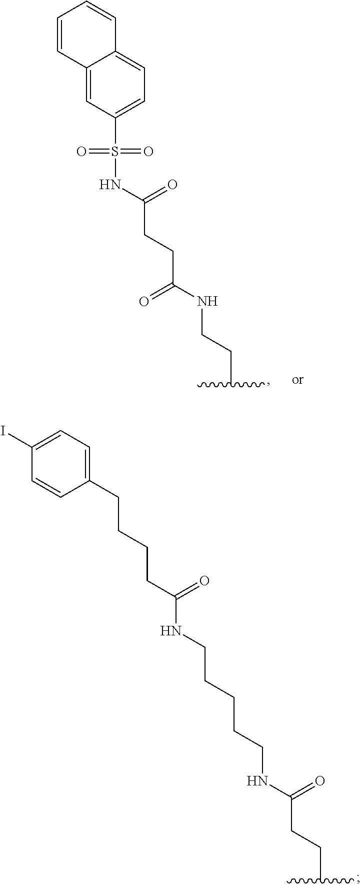 [SMILES: *CCNC(=O)CCC(=O)NS(=O)(=O)c1ccc2ccccc2c1 ; *CCC(=O)NCCCCCNC(=O)CCCCc1ccc(I)cc1]

R^{7b1} is H or C₁-C₃ alkyl;
R^{7b2} is (CH₂CH₂O)ₓCH₂CH₂COOH or (CH₂)_{y}COOH;
R^{7c} is H or C₁-C₃ alkyl; or
R^{7b} and R⁷, together with the nitrogen to which they are attached, combine to form a 3-6 membered heterocycloalkyl group optionally substituted with amino, COOH, or C₁-C₃ alkyl that is optionally substituted with OH or COOH;
R⁸ is H, C₁-C₃ haloalkyl, COOH, C(═O)NH—R^{8a},

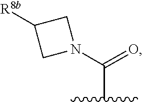

or C(═O)NH(CH₂)₂NHC(═O)—R^{8c};
R^{8a} is C₁-C₁₀ alkyl substituted with COOH or SO₃H, or C₁-C₃ alkoxy optionally substituted with amino;
R^{8b} is COOH or amino,
R^{8c} is (CH₂CH₂O)ₓCH₂CH₂COOH or (CH₂)_{y}COOH;
wherein when R⁷ is H, then R⁸ is C₁-C₃ haloalkyl, COOH, C(═O)NH—R^{8a},

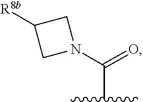

or C(═O)NH(CH₂)₂NHC(═O)—R^{8c}; and
wherein when R⁸ is H, then R⁷ is C₁-C₃ haloalkyl, OR^{7a},

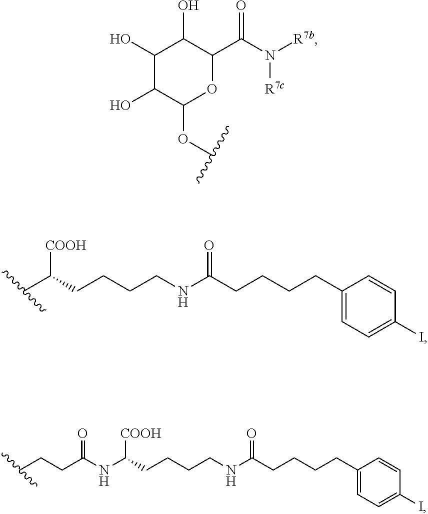

or C₁-C₃ alkyl optionally substituted with COOH.

In one aspect, described herein is a compound that has the structure of Formula (I), or a pharmaceutically acceptable salt thereof:

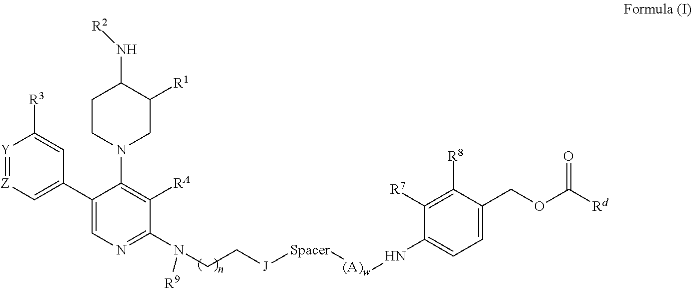

wherein:
J is a bond or NR⁶;
n is an integer selected from 1-5;
R^{A} is

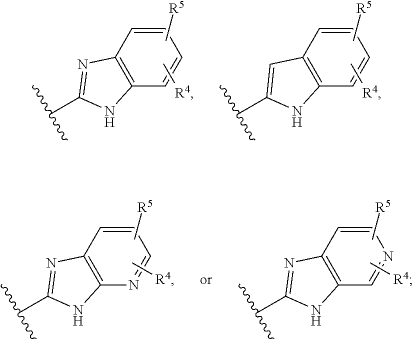

Y is N or CH;
Z is N or CR¹⁰;
R¹ is H;
R² is H or C₁-C₆ alkyl; or
R¹ and R², together with the N and C to which they are attached, combine to form a heterocycloalkyl;
R³ is C₁-C₆ alkyl or C₁-C₆ alkoxy;
R⁴ is H, halogen, CN, or C₁-C₆ alkoxy;
R⁵ is H, halogen, or CN;
R⁶ is H or C₁-C₆ alkyl;
R⁹ is H or C₁-C₆ alkyl;
R¹⁰ is H or halogen;
Spacer is a spacer group;
each A is independently selected from natural or unnatural amino acids, wherein any free amine of an amino acid is optionally independently substituted with CH₃;
w is an integer selected from 1-6;
R⁷ is H, C₁-C₆ alkyl, C₁-C₆ haloalkyl, OR^{7a}, or

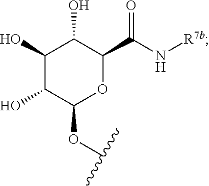

R^{7a} is H; or C₁-C₆ alkyl optionally substituted with amino, COOH, or OH;
R^{7b} is H; C₁-C₂₀ alkyl substituted with COOR^{7b1}, amino or SO₃H; (CH₂)₂NHC(═O)—R^{7b2};

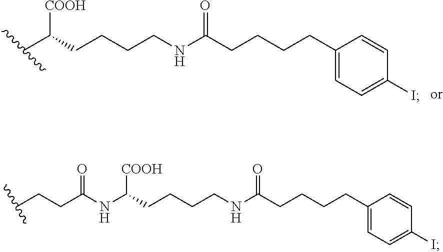 [SMILES: *[C@@H](CCCCNC(=O)CCCCc1ccc(I)cc1)C(=O)O ; "or" ; *CCC(=O)N[C@@H](CCCCNC(=O)CCCCc1ccc(I)cc1)C(=O)O]

R^{7b1} is H or C₁-C₆ alkyl;
R^{7b2} is (CH₂CH₂O)ₓCH₂CH₂COOH or (CH₂)_{y}COOH;
R⁸ is H, C₁-C₆ haloalkyl, COOH, C(═O)NH—R^{8a},

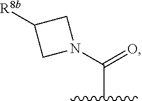

or C(═O)NH(CH₂)₂NHC(═O)—R^{8c};
R^{8a} is C₁-C₂₀ alkyl substituted with COOH or SO₃H; or C₁-C₆ alkoxy optionally substituted with amino;
R^{8b} is COOH or amino;
R^{8c} is (CH₂CH₂O)ₓCH₂CH₂COOH or (CH₂)_{y}COOH;
each x is independently an integer selected from 1-10;
each y is independently an integer selected from 1-20;
R^{d} is the residue of a bioactive molecule of the formula R^{d}—H;
wherein when R⁷ is H, then R⁸ is C₁-C₆ haloalkyl, COOH, C(═O)NH—R^{8A},

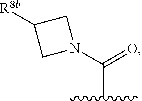

or C(═O)NH(CH₂)₂NHC(═O)—R^{8c}; and
wherein when R⁸ is H, then R⁷ is C₁-C₆ alkyl, C₁-C₆ haloalkyl, OR^{7a},

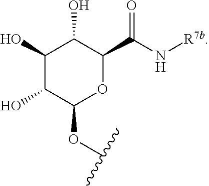

In some embodiments, R⁷ is H, C₁-C₃ alkyl, C₁-C₃ haloalkyl, OR^{7a}, or

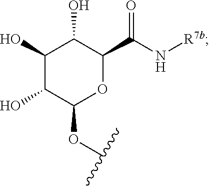

R^{7a} is H; or C₁-C₃ alkyl optionally substituted with amino, COOH, or OH;
R^{7b} is H; C₁-C₁₄ alkyl substituted with COOR^{7b1}, amino, or SO₃H; (CH₂)₂NHC(═O)—R^{7b2};

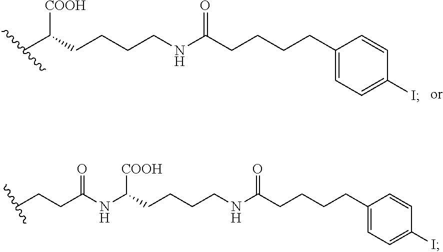 [SMILES: *[C@@H](CCCCNC(=O)CCCCc1ccc(I)cc1)C(=O)O ; "or" ; *CCC(=O)N[C@@H](CCCCNC(=O)CCCCc1ccc(I)cc1)C(=O)O]

R^{7b1} is H or C₁-C₃ alkyl;
R^{7b2} is (CH₂CH₂O)ₓCH₂CH₂COOH, or (CH₂)_{y}COOH;
R⁸ is H, C₁-C₃ haloalkyl, COOH, C(═O)NH—R^{8a},

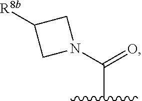

or C(═O)NH(CH₂)₂NHC(═O)—R^{8c};
R^{8a} is C₁-C₁₀ alkyl substituted with COOH or SO₃H; or C₁-C₃ alkoxy optionally substituted with amino;
R^{8b} is COOH or amino;
R^{8c} is (CH₂CH₂O)ₓCH₂CH₂COOH or (CH₂)_{y}COOH;
wherein when R⁷ is H, then R⁸ is C₁-C₃ haloalkyl, COOH, C(═O)NH—R^{8a},

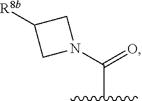

or C(═O)NH(CH₂)₂NHC(═O)—R^{8c}; and
wherein when R⁸ is H, then R⁷ is C₁-C₃ alkyl, C₁-C₃ haloalkyl, OR^{7a}, or

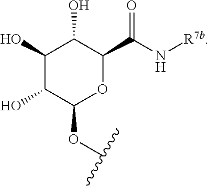

In some embodiments. R⁷ is methyl, trifluoromethyl, OR^{7a}, or

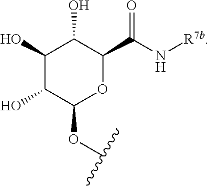

In some embodiments, R⁷ is

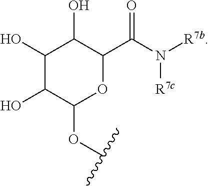

In some embodiments, R⁷ is OR^{7a} or

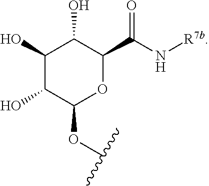

In some embodiments, R⁸ is trifluoromethyl, COOH, O(═O)NH—R^{8a},

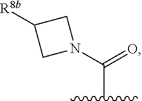

or C(═O)NH(CH₂)₂NHC(═O)—R^{8c}.

In some embodiments,

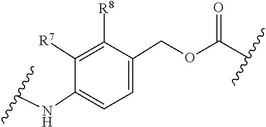

### of Formula (I) is:

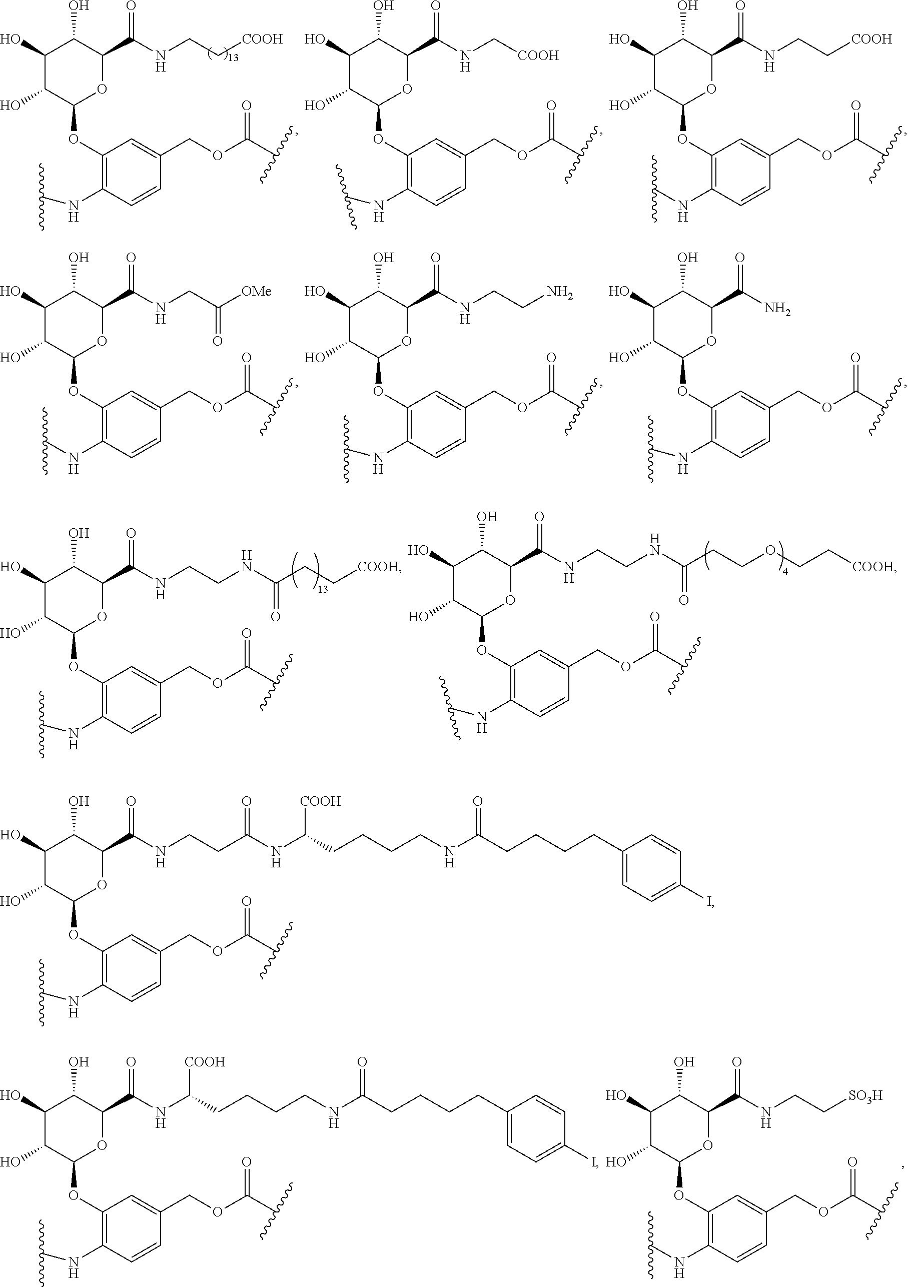

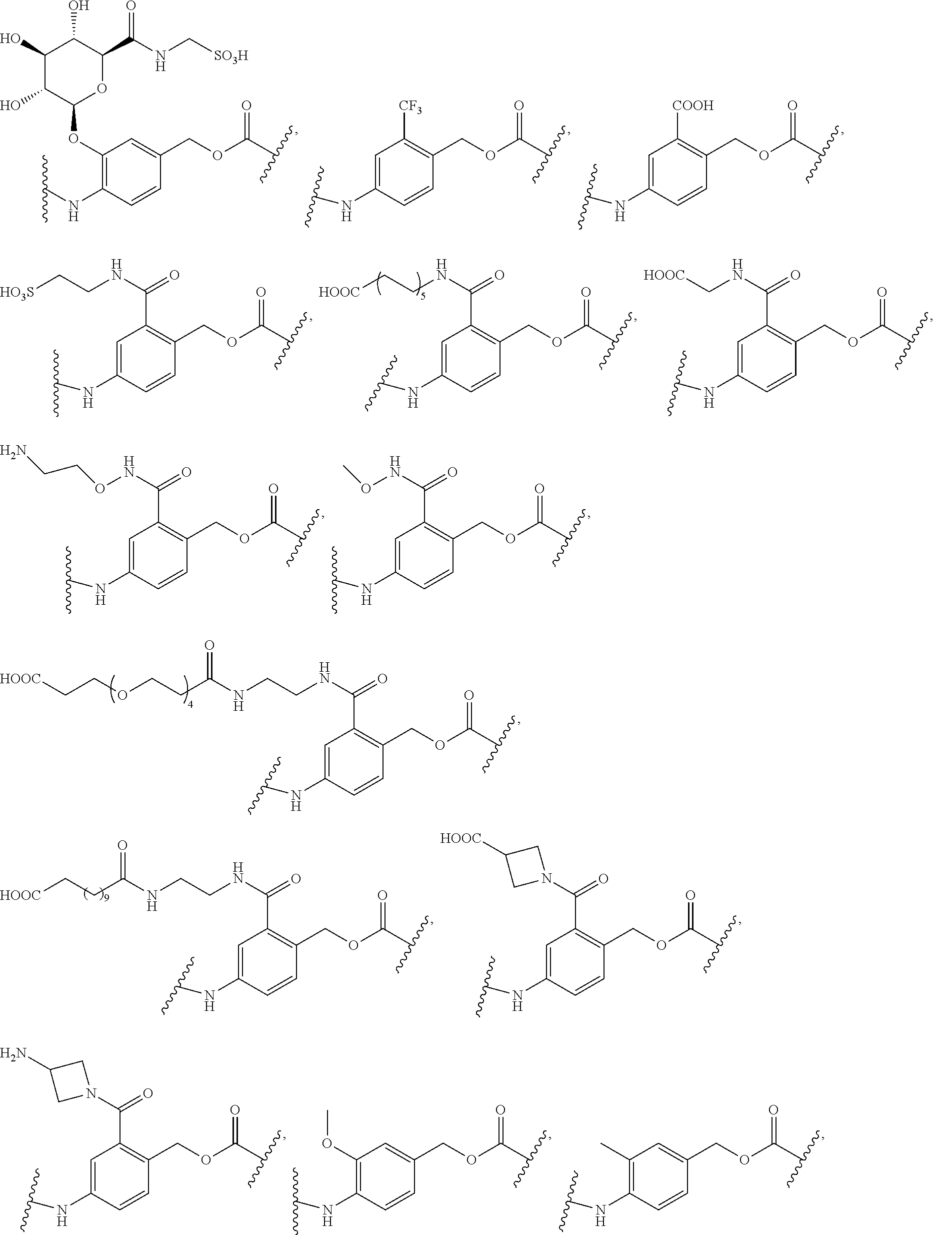

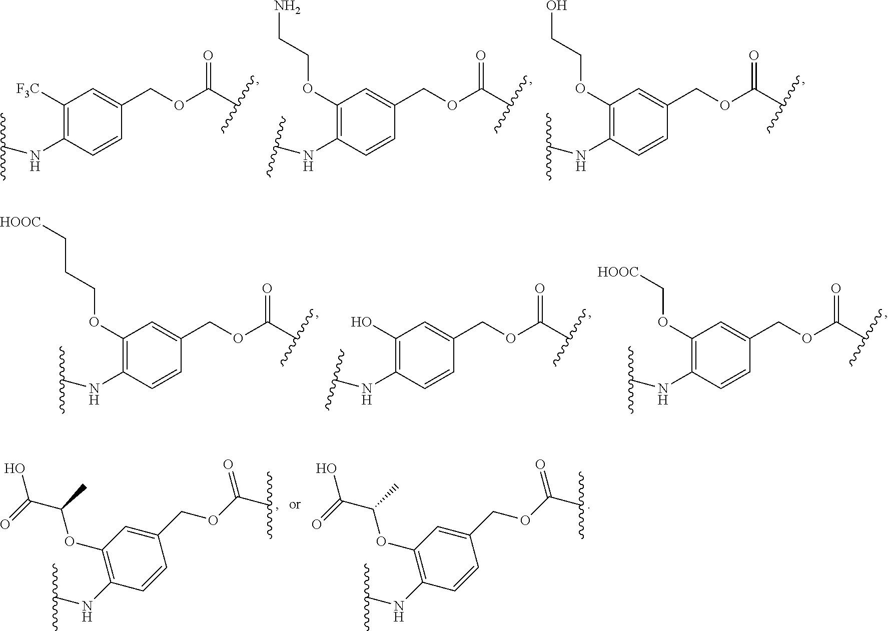

In some embodiments,

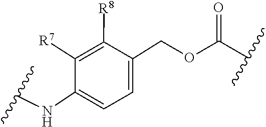

### of Formula (I) is:

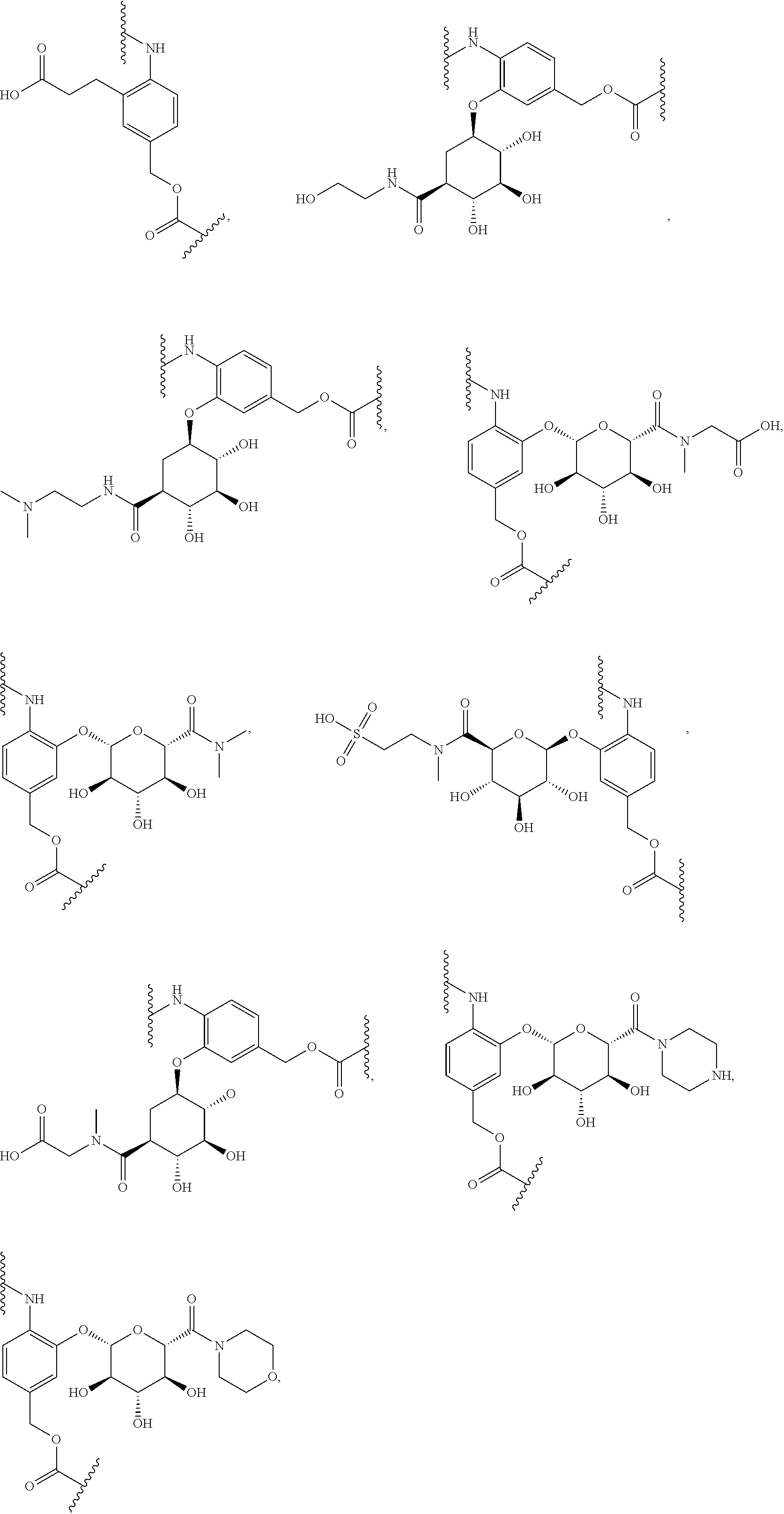

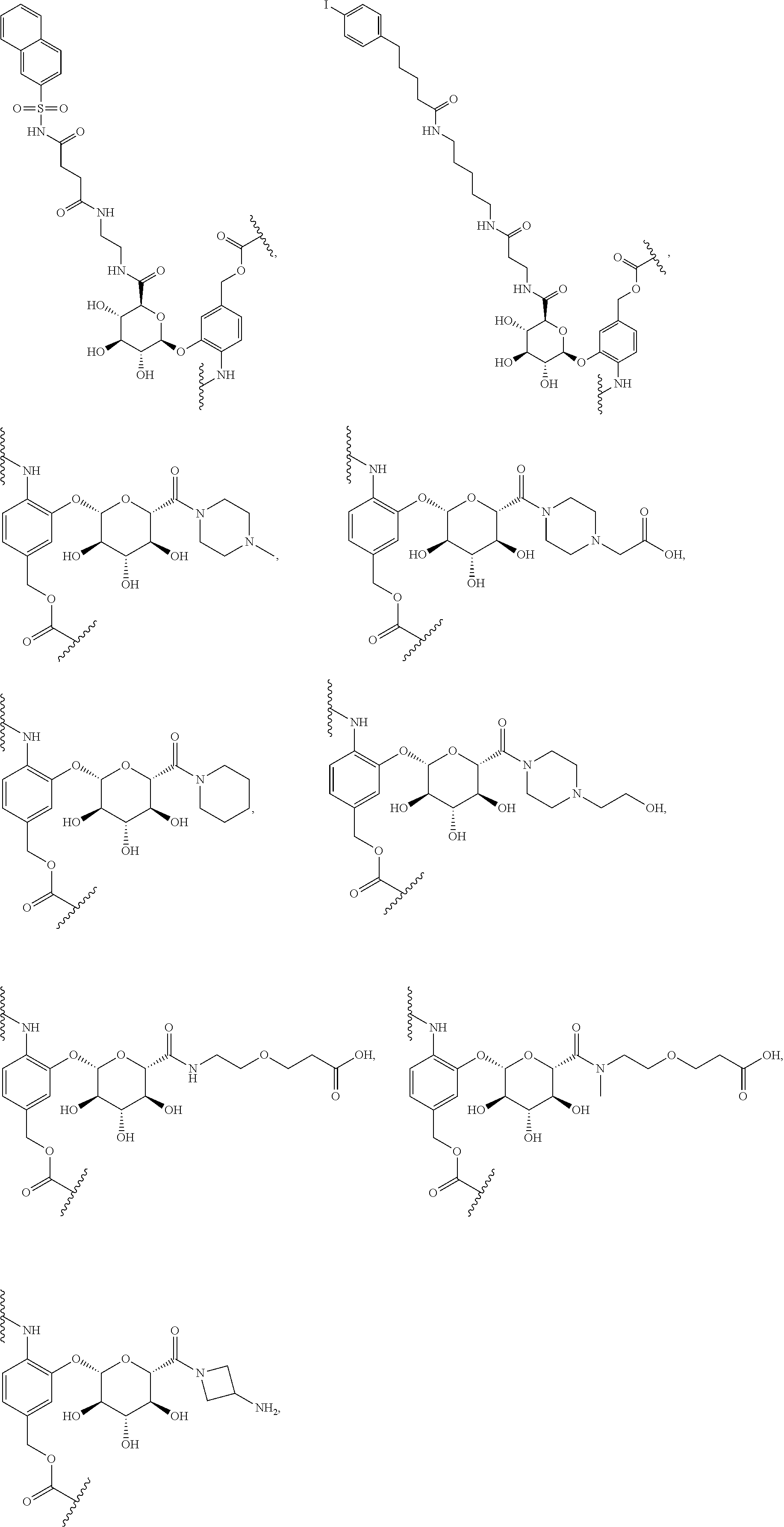

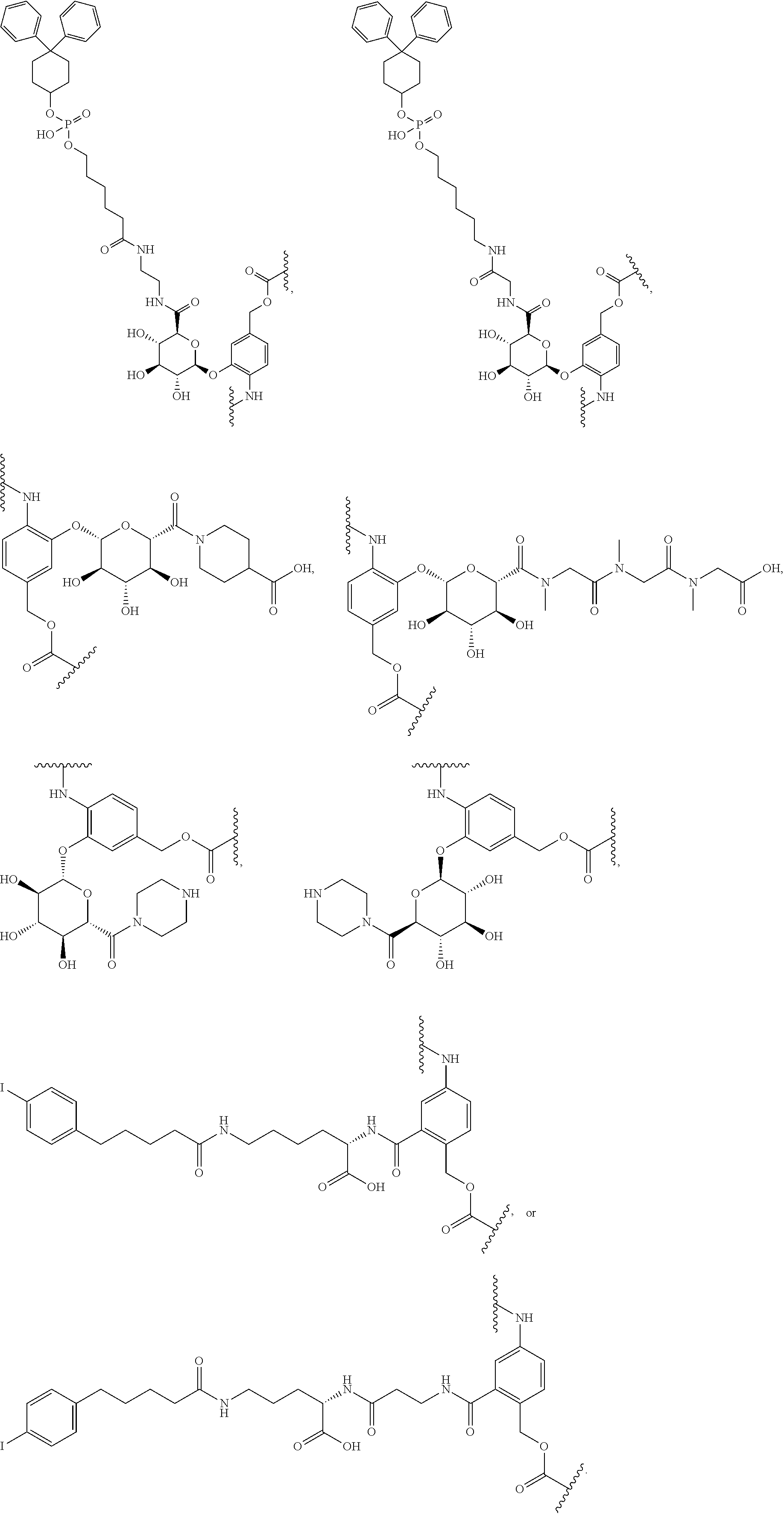

In some embodiments,

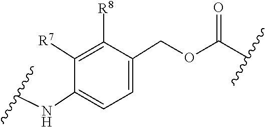

### of Formula (I) is:

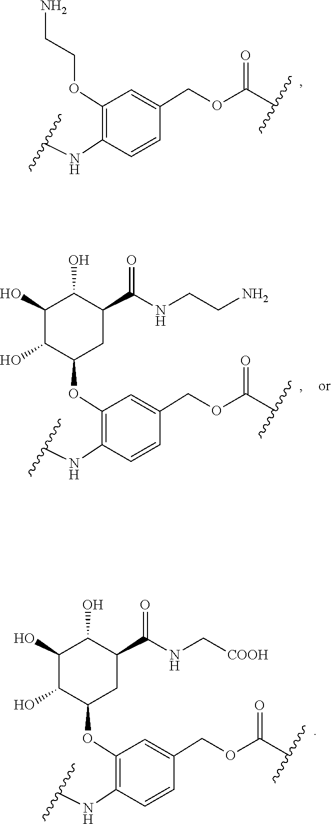 [SMILES: *Nc1ccc(COC(*)=O)cc1OCCN ; *Nc1ccc(COC(*)=O)cc1O[C@@H]1C[C@H](C(=O)NCCN)[C@@H](O)[C@H](O)[C@H]1O ; *Nc1ccc(COC(*)=O)cc1O[C@@H]1C[C@H](C(=O)NCC(=O)O)[C@@H](O)[C@H](O)[C@H]1O]

In some embodiments, each A is independently selected from natural or unnatural amino acids, wherein any free amine of an amino acid is optionally independently substituted with CH₃, wherein the natural or unnatural amino acids are selected from alanine (Ala), Ala(SO₃H), 3-(1-piperidinyl)alanine, cyclohexylalanine, arginine (Arg), asparagine (Asn), aspartate (Asp), cysteine (Cys), glutamine (Gin), glutamatic acid (Glu), glycine (Gly), leucine (Leu), lysine(Lys), methionine (Met), phenylalanine (Phe), homophenylalanine, proline (Pro), serine (Ser), 3-homoserine, tyrosine(Tyr), Tyr(SO₃H), tryptophan (Trp), valine (Val), citrulline, β-alanine, β3-homoserine, β3-homolysine, and β3-homoglutamic acid.

In some embodiments, each A is independently selected from natural or unnatural amino acids, wherein any free amine of an amino acid is optionally independently substituted with CH₃, wherein the natural or unnatural amino acids are selected from alanine (Ala), Ala(SO₃H), glutamine (Gin), glutamatic acid (Glu), glycine (Gly), leucine (Leu), serine (Ser), tryptophan (Trp), valine (Val), and citrulline.

In some embodiments, w is an integer selected from 2 or 3.

In some embodiments, (A)_{w} is valine-citrulline, valine-alanine, alanine(SO₃H)-valine-alanine, glutamic acid-valine-citrulline, glycine-valine-citrulline, glutamine-valine-citrulline, alanine(SO₃H)-valine-citrulline, serine-valine-citrulline, or tryptophane-valine-citrulline.

In some embodiments, (A)_{w} is valine-citrulline, valine-alanine, or glutamic acid-valine-citrulline.

In some embodiments,

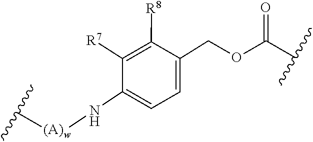

### of Formula (I) is:

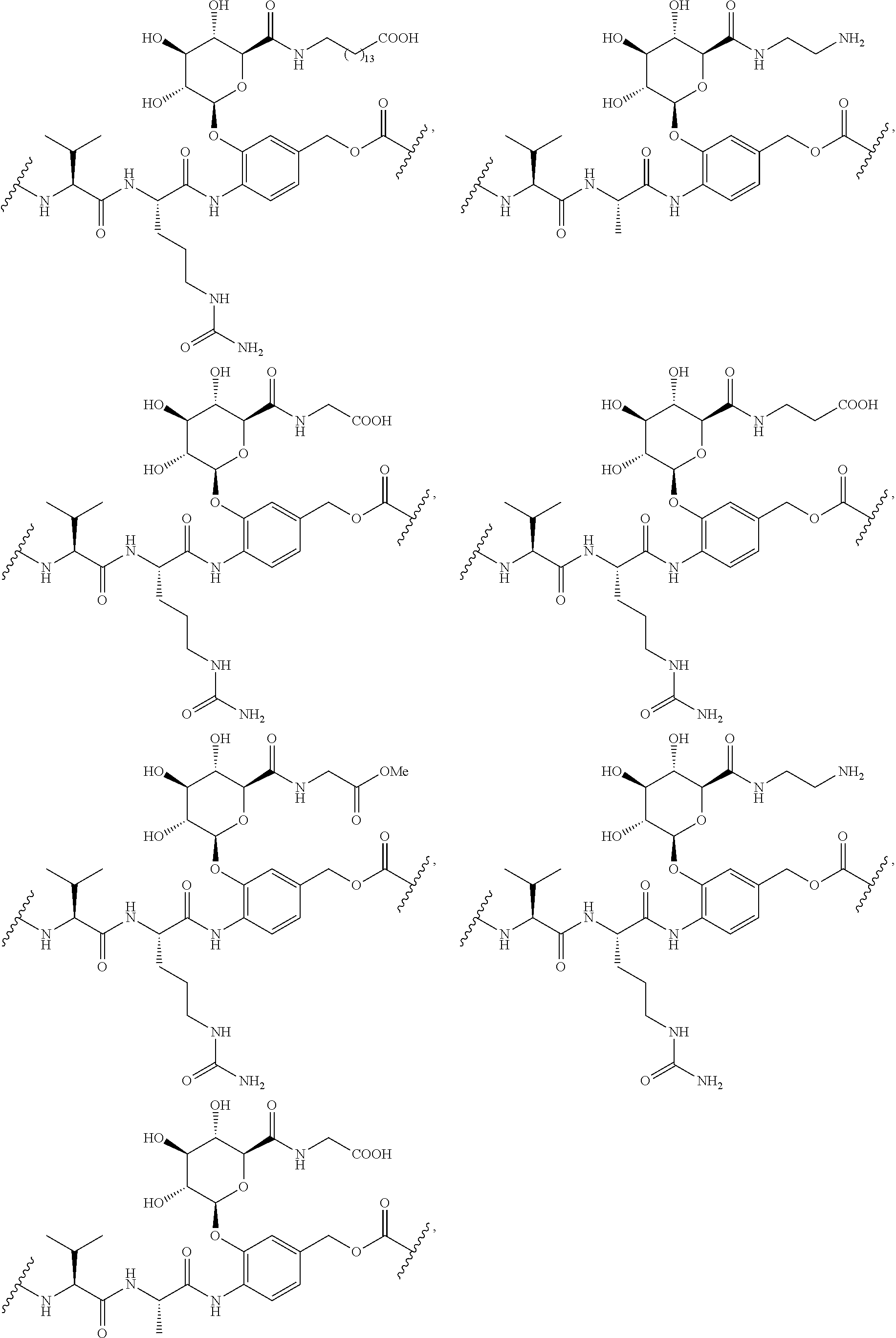

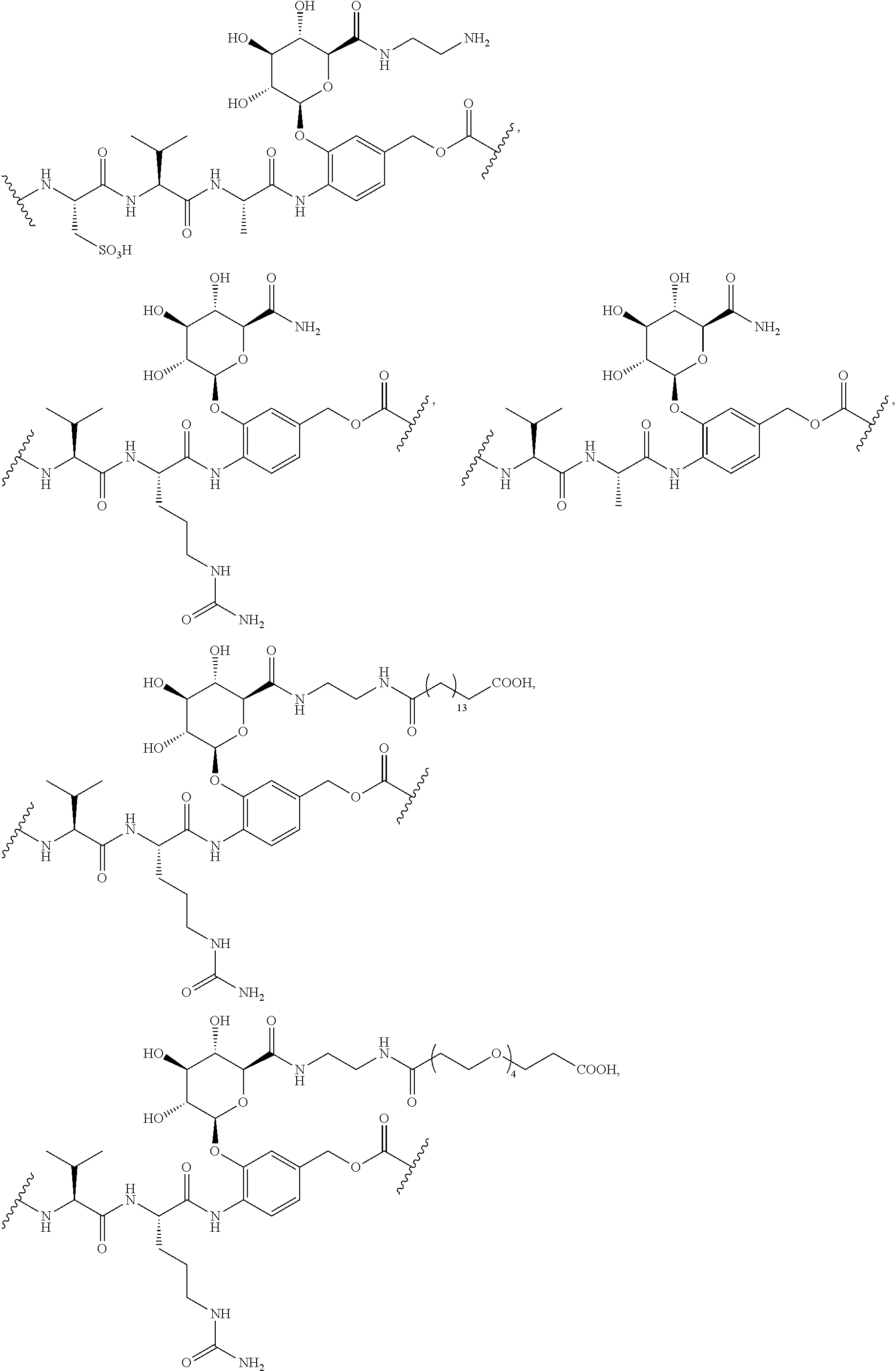

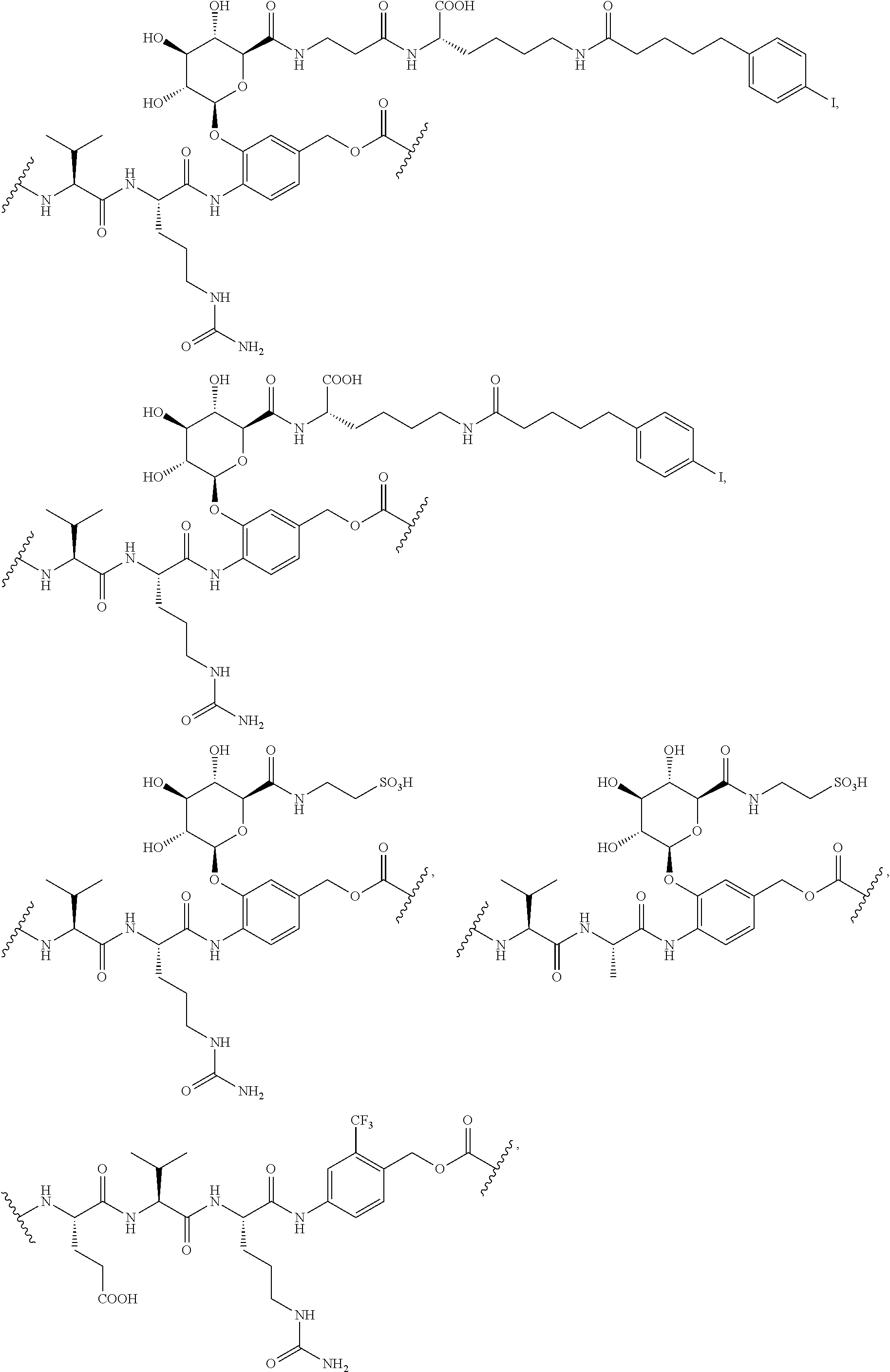

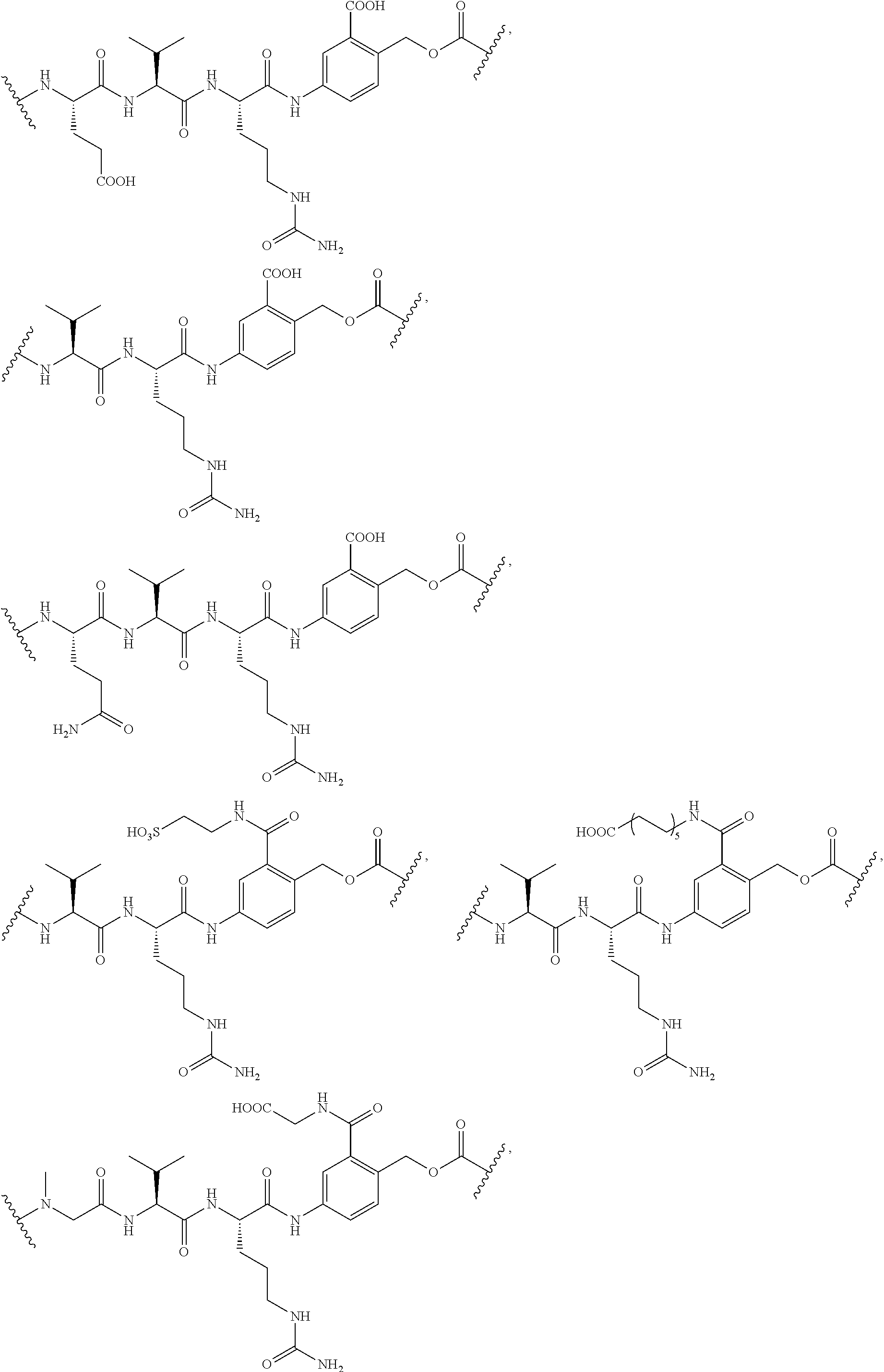

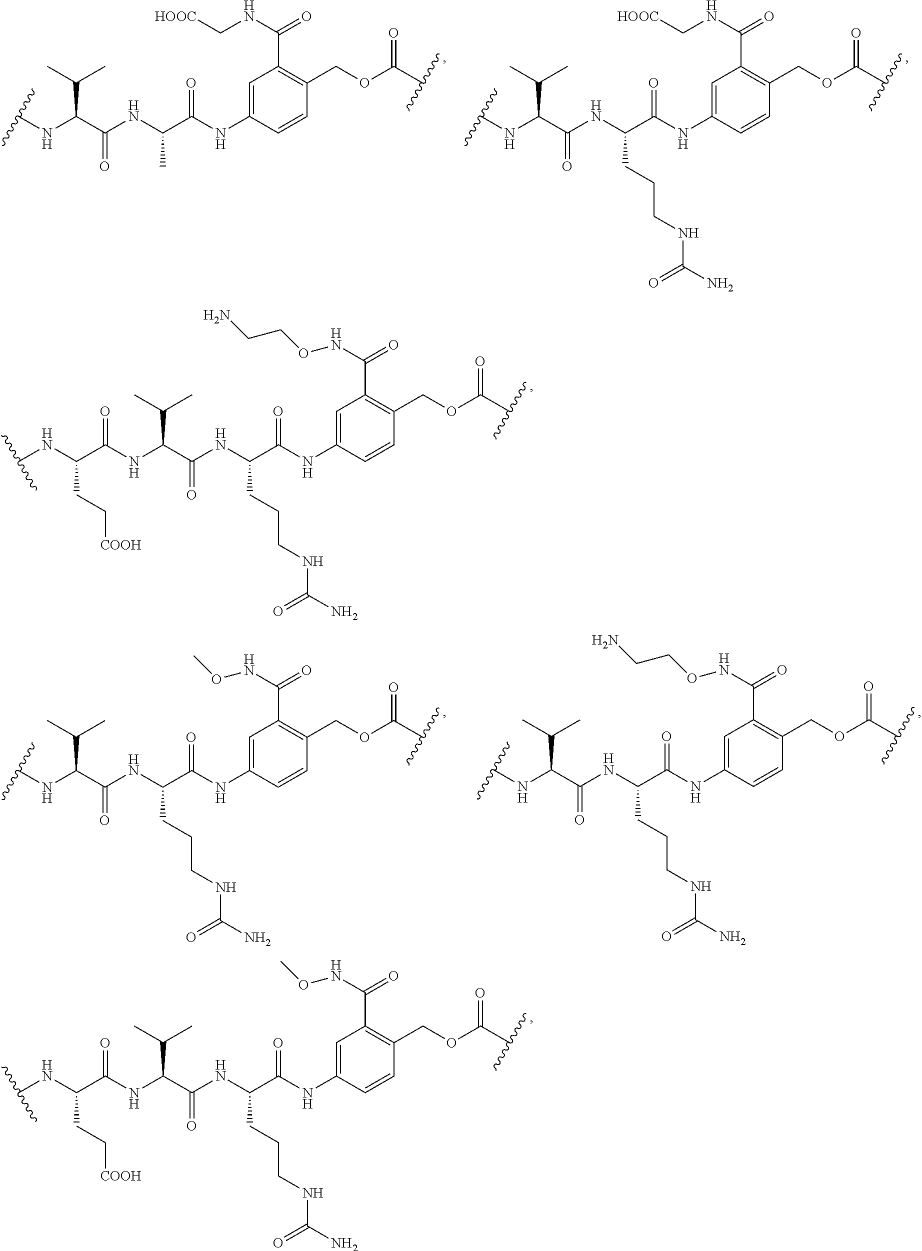

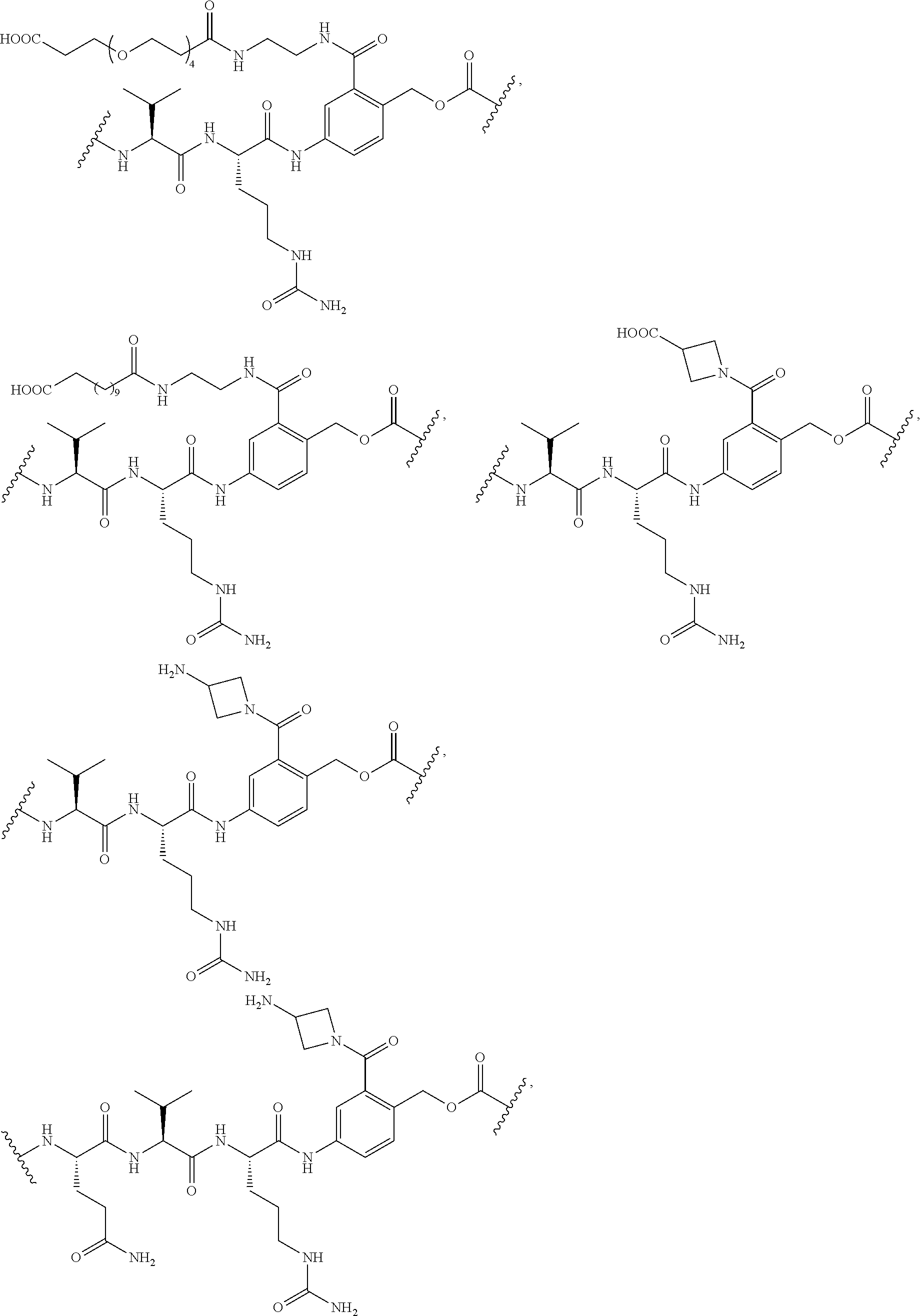

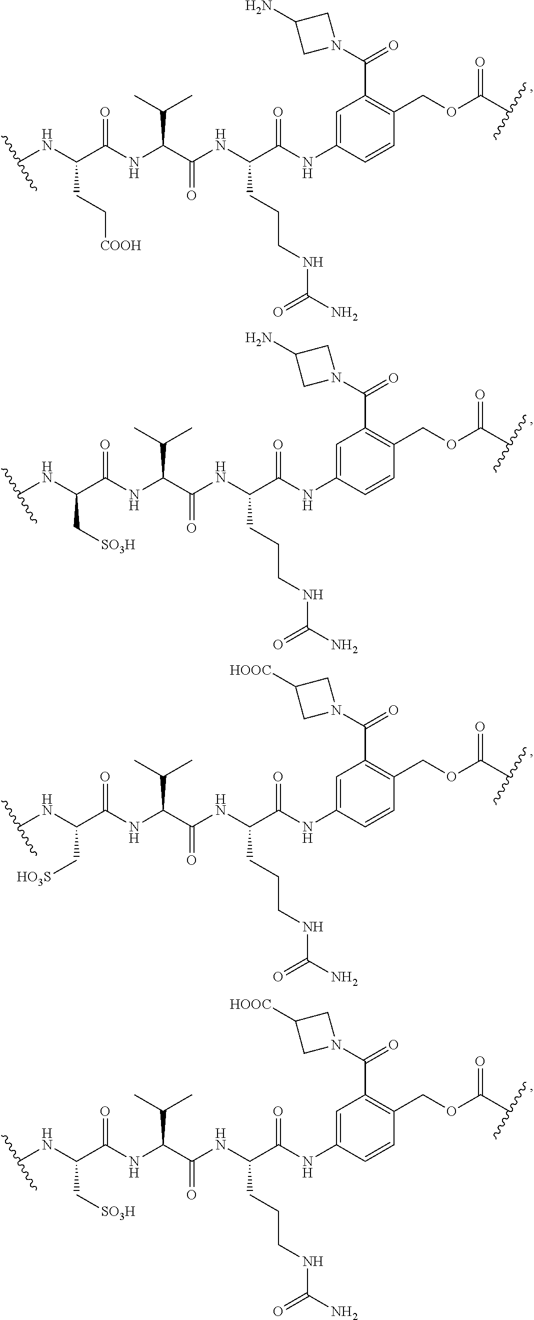

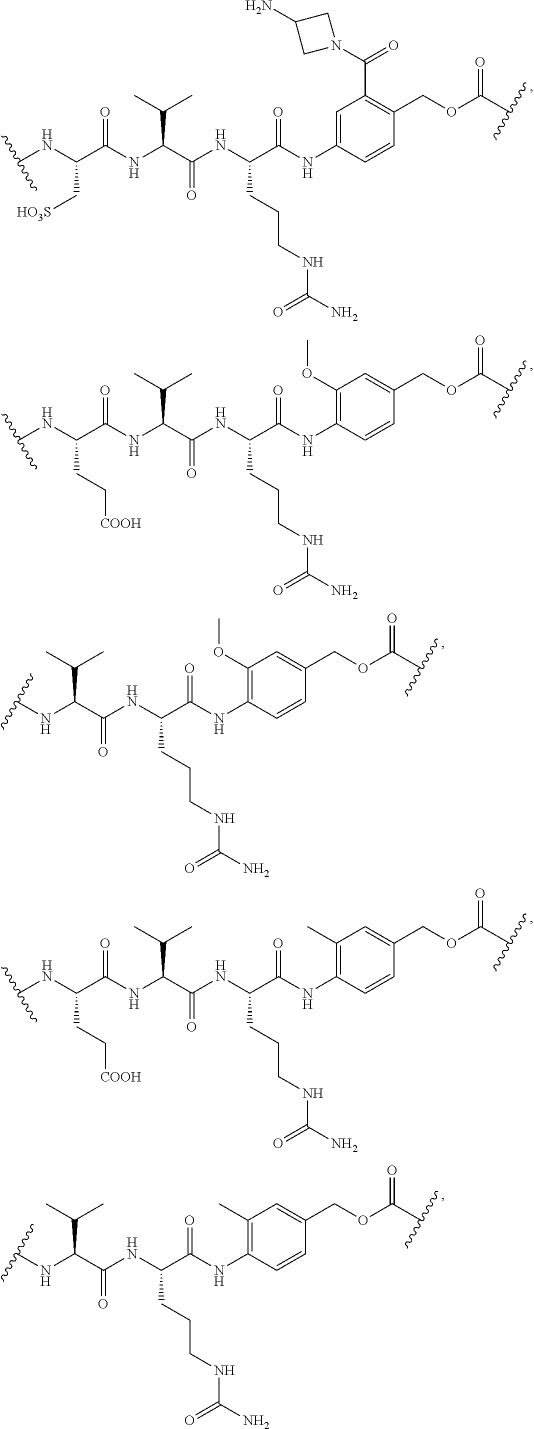

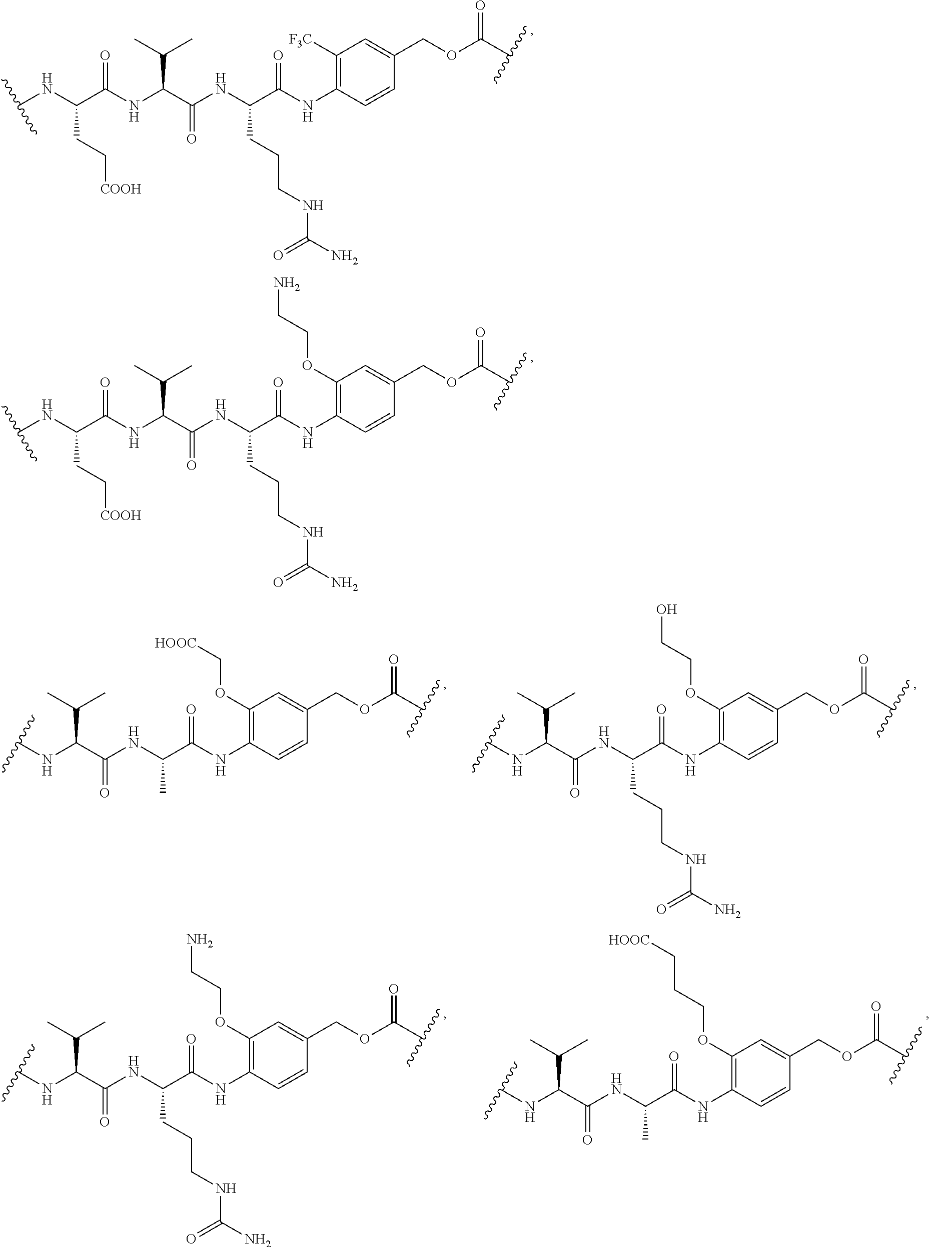

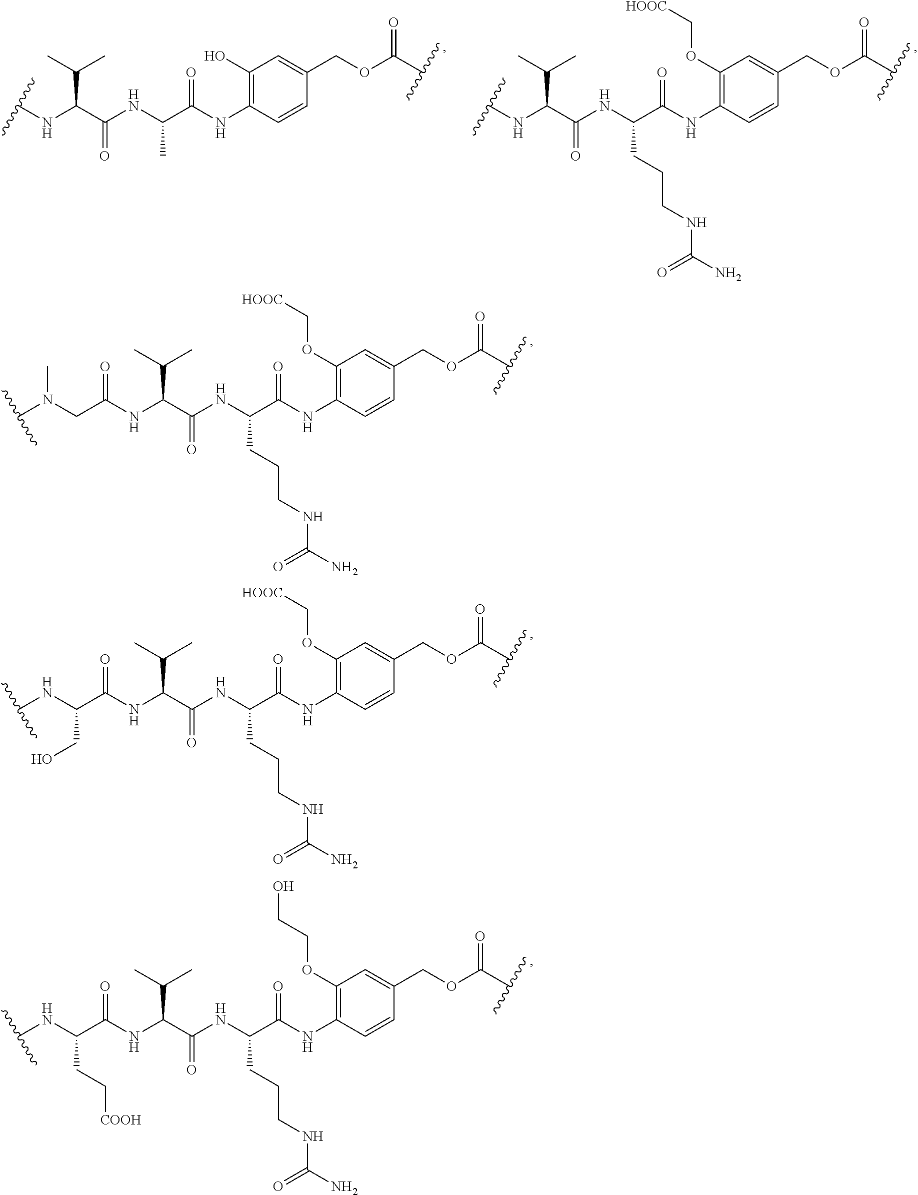

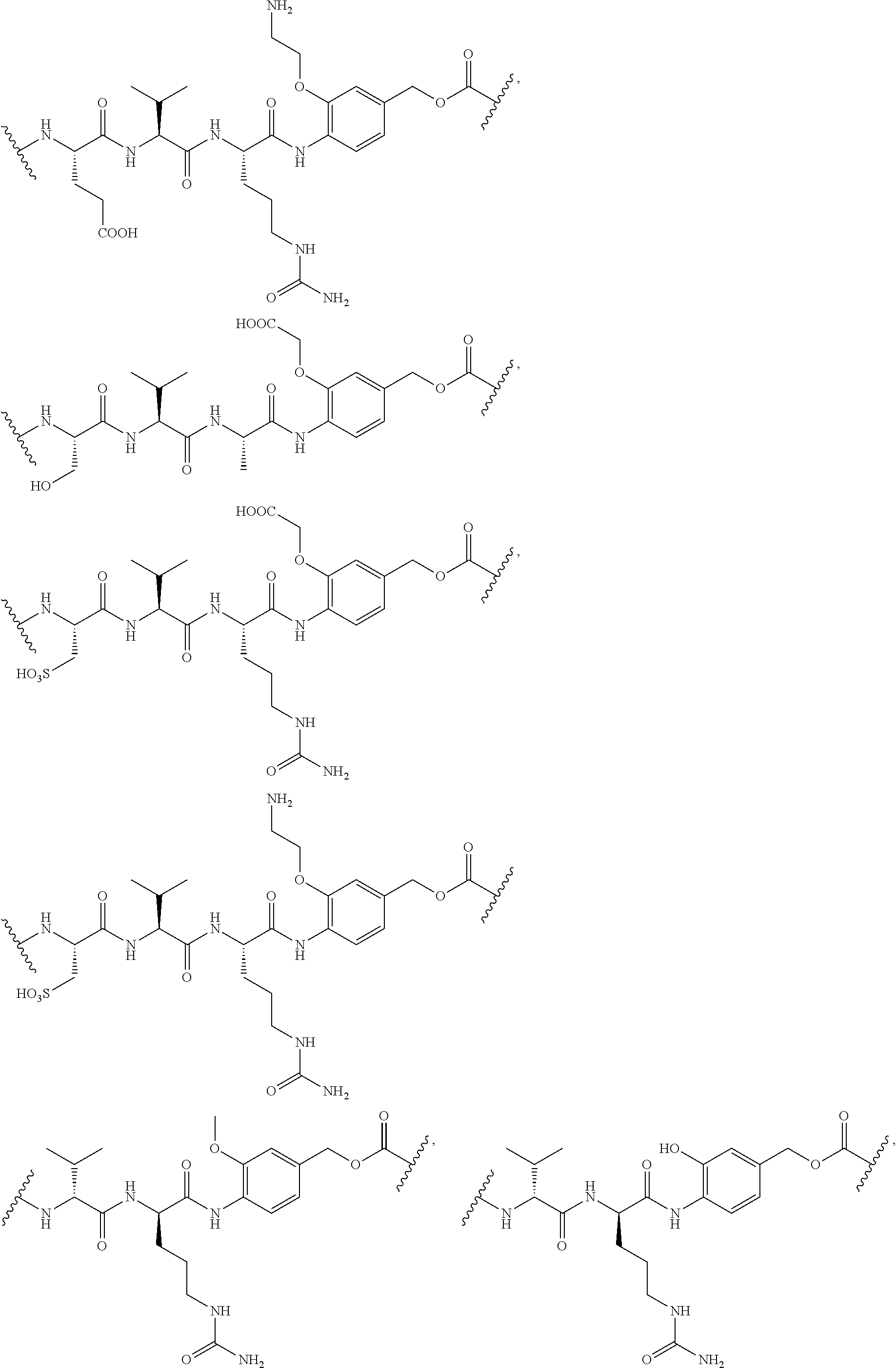

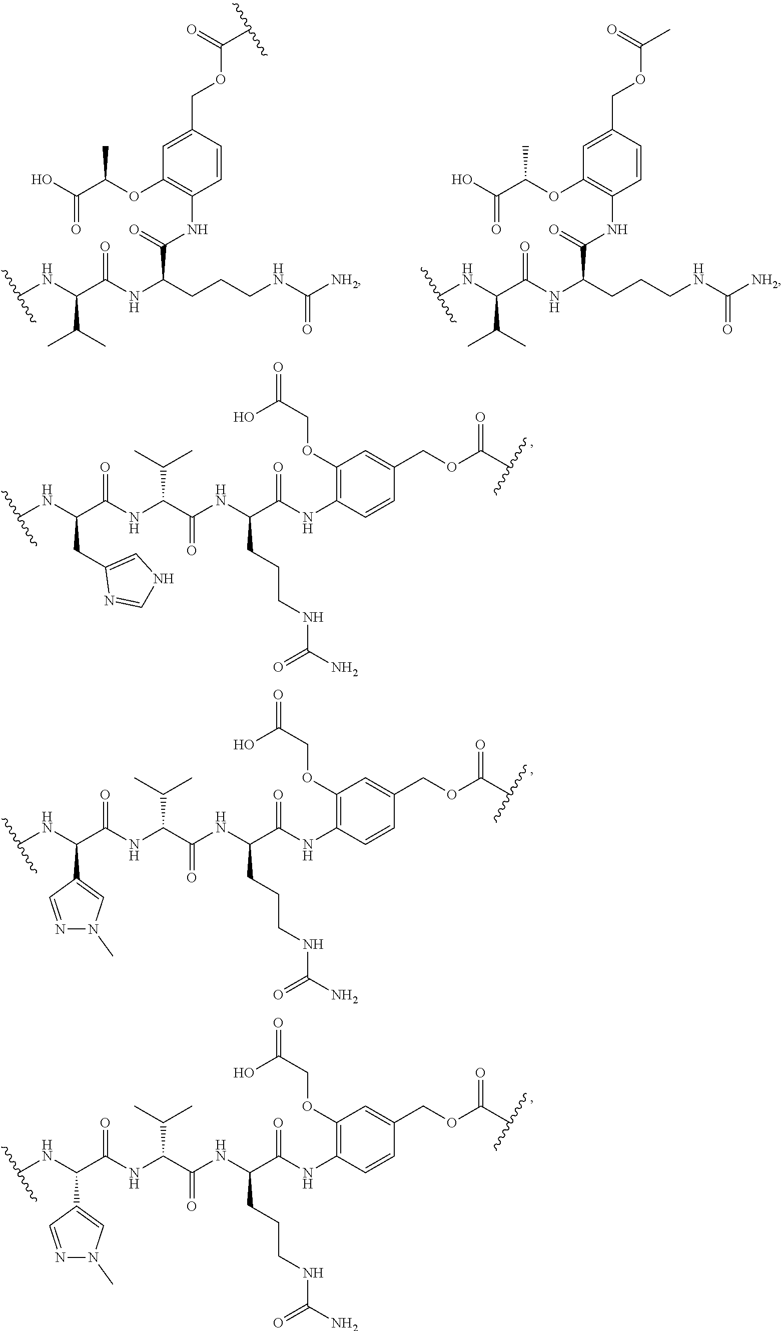

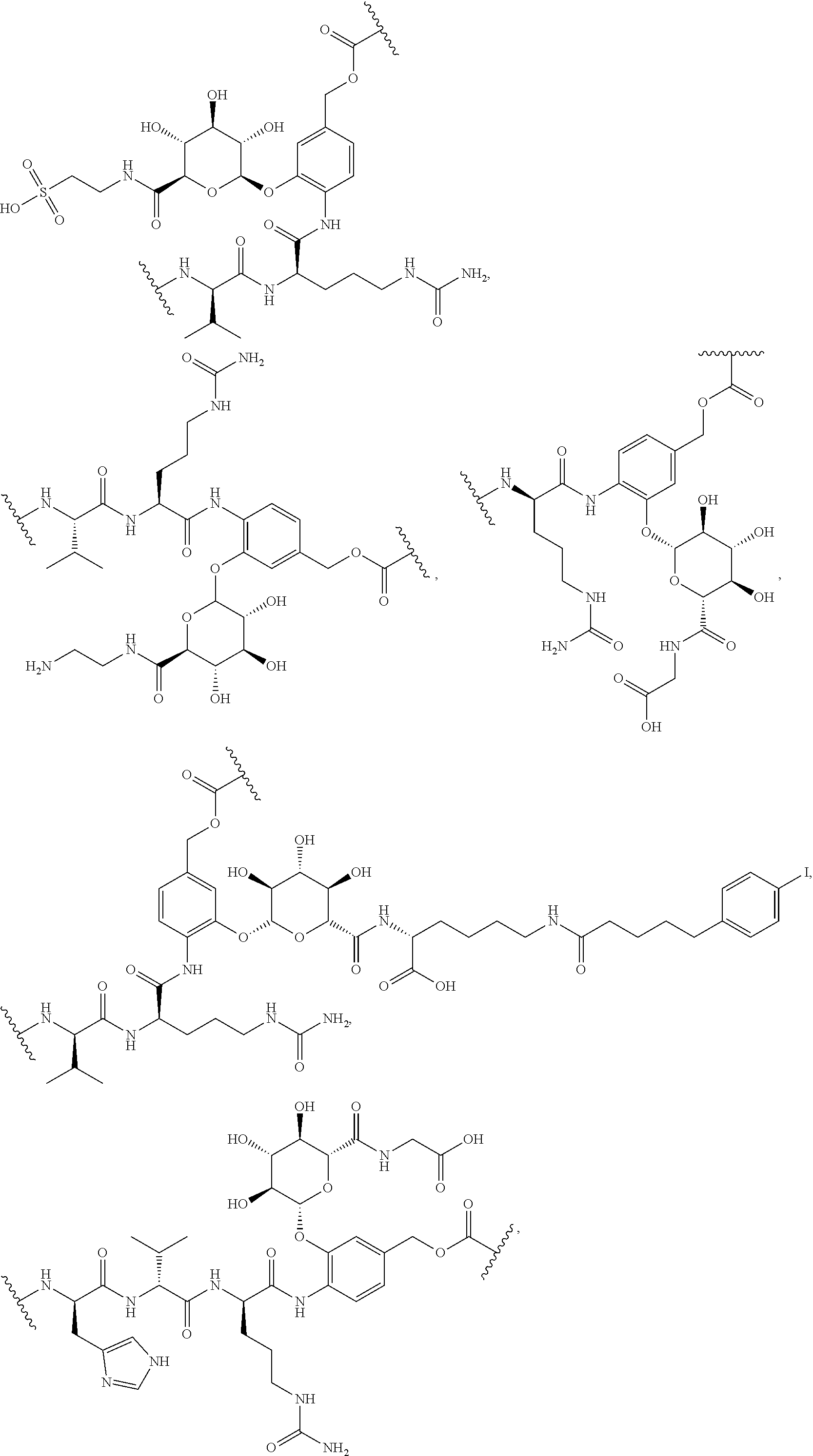

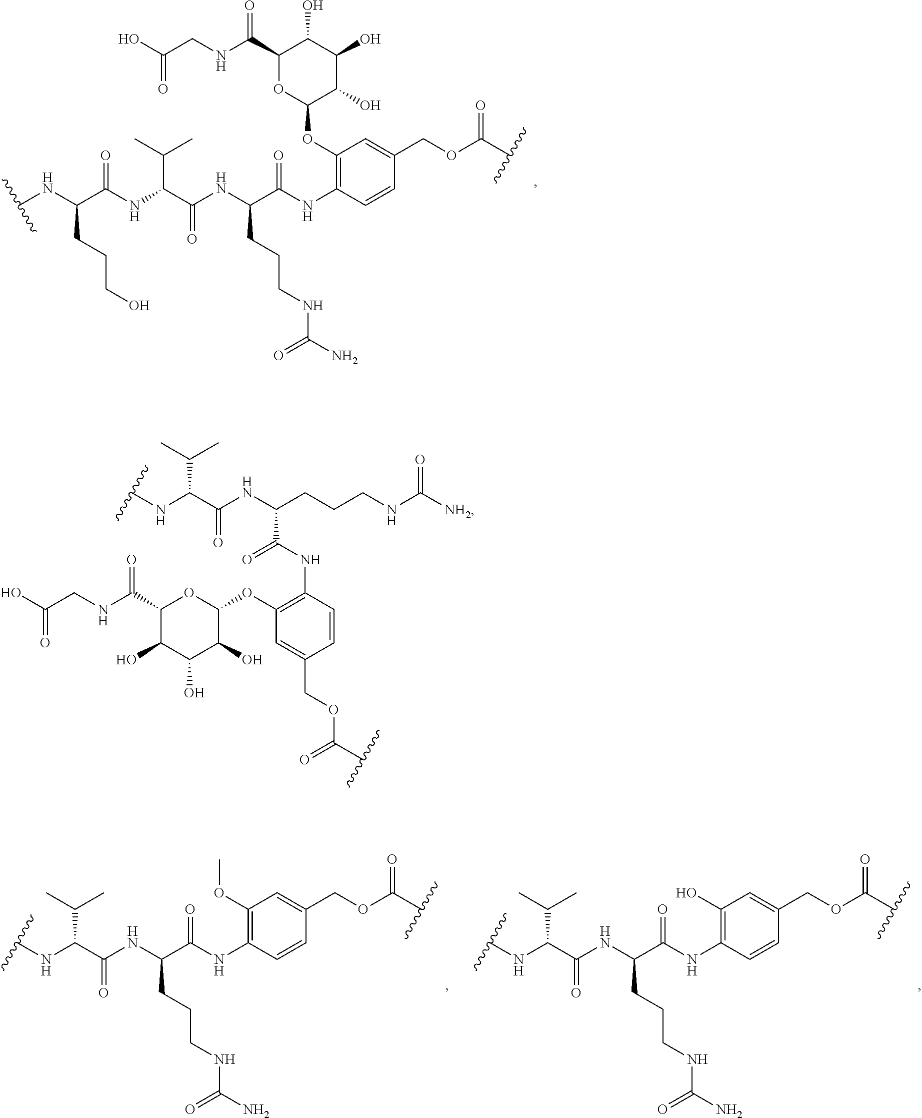 [SMILES: *N[C@H](CCCO)C(=O)N[C@@H](C(=O)N[C@H](CCCNC(N)=O)C(=O)Nc1ccc(COC(*)=O)cc1O[C@H]1O[C@@H](C(=O)NCC(=O)O)[C@H](O)[C@@H](O)[C@@H]1O)C(C)C ; *N[C@@H](C(=O)N[C@H](CCCNC(N)=O)C(=O)Nc1ccc(COC(*)=O)cc1O[C@H]1O[C@@H](C(=O)NCC(=O)O)[C@H](O)[C@@H](O)[C@@H]1O)C(C)C ; *N[C@@H](C(=O)N[C@H](CCCNC(N)=O)C(=O)Nc1ccc(COC(*)=O)cc1OC)C(C)C ; *N[C@@H](C(=O)N[C@H](CCCNC(N)=O)C(=O)Nc1ccc(COC(*)=O)cc1O)C(C)C]

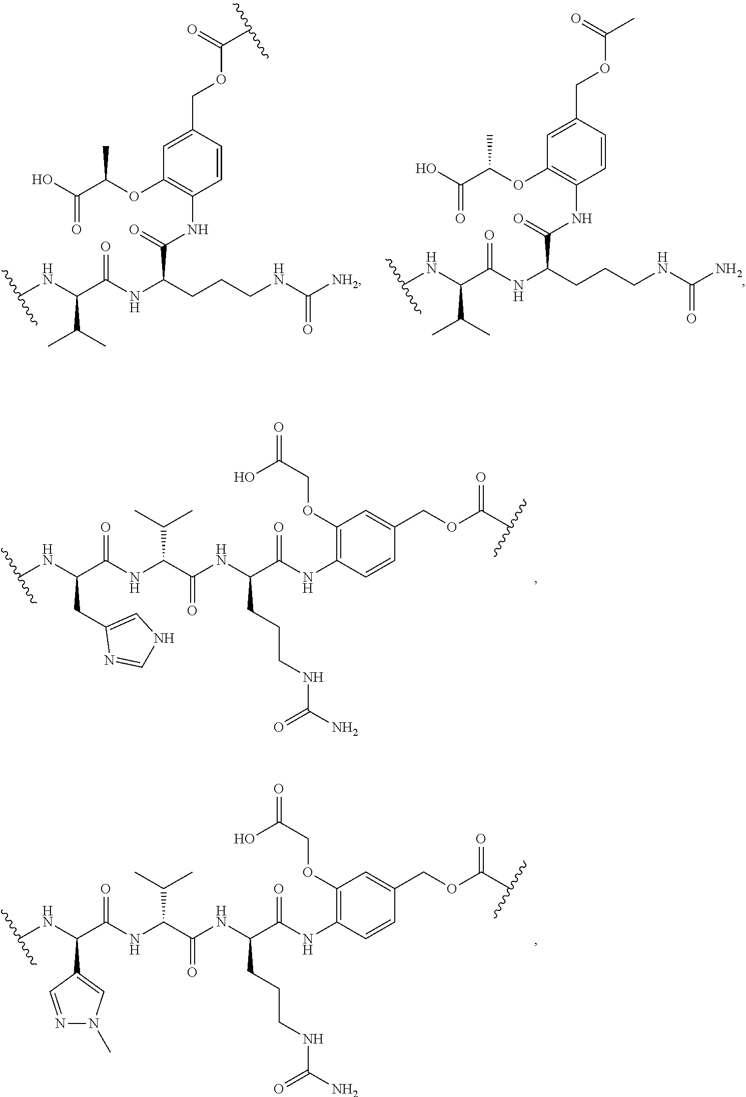

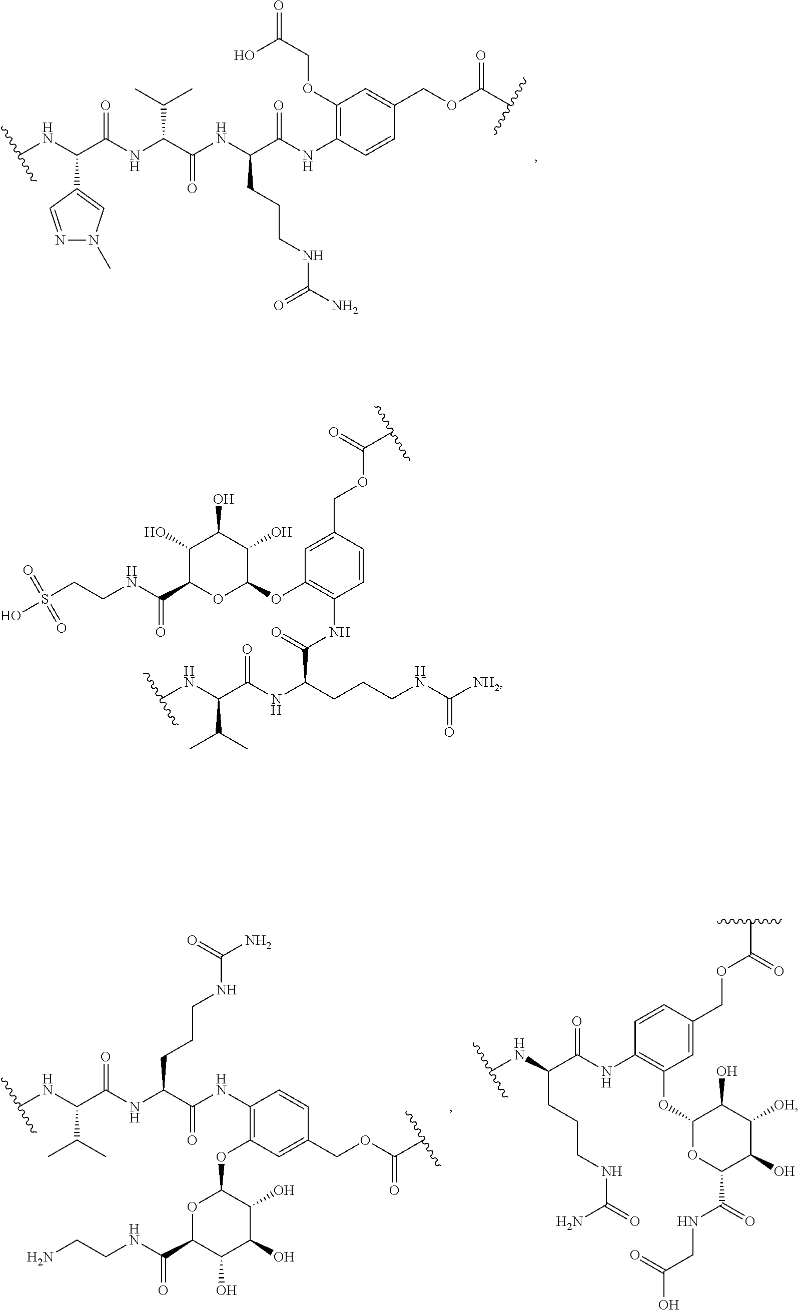 [SMILES: *N[C@H](C(=O)N[C@@H](C(=O)N[C@H](CCCNC(N)=O)C(=O)Nc1ccc(COC(*)=O)cc1OCC(=O)O)C(C)C)c1cnn(C)c1 ; *N[C@@H](C(=O)N[C@H](CCCNC(N)=O)C(=O)Nc1ccc(COC(*)=O)cc1O[C@H]1O[C@@H](C(=O)NCCS(=O)(=O)O)[C@H](O)[C@@H](O)[C@@H]1O)C(C)C ; *N[C@H](C(=O)N[C@@H](CCCNC(N)=O)C(=O)Nc1ccc(COC(*)=O)cc1O[C@@H]1O[C@H](C(=O)NCCN)[C@@H](O)[C@H](O)[C@H]1O)C(C)C ; *N[C@H](CCCNC(N)=O)C(=O)Nc1ccc(COC(*)=O)cc1O[C@H]1O[C@@H](C(=O)NCC(=O)O)[C@H](O)[C@@H](O)[C@@H]1O]

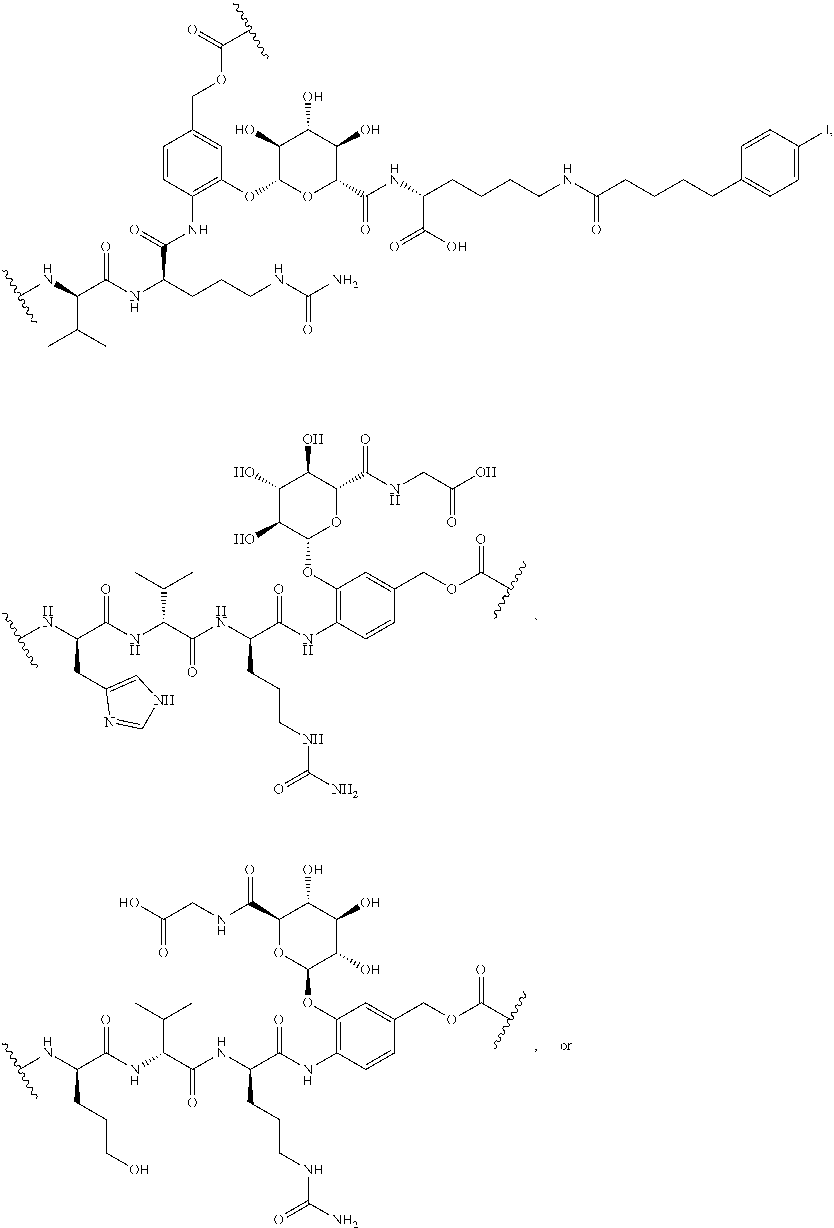 [SMILES: *N[C@@H](C(=O)N[C@H](CCCNC(N)=O)C(=O)Nc1ccc(COC(*)=O)cc1O[C@H]1O[C@@H](C(=O)N[C@H](CCCCNC(=O)CCCCc2ccc(I)cc2)C(=O)O)[C@H](O)[C@@H](O)[C@@H]1O)C(C)C ; *N[C@H](Cc1c[nH]cn1)C(=O)N[C@@H](C(=O)N[C@H](CCCNC(N)=O)C(=O)Nc1ccc(COC(*)=O)cc1O[C@H]1O[C@@H](C(=O)NCC(=O)O)[C@H](O)[C@@H](O)[C@@H]1O)C(C)C ; *N[C@H](CCCO)C(=O)N[C@@H](C(=O)N[C@H](CCCNC(N)=O)C(=O)Nc1ccc(COC(*)=O)cc1O[C@H]1O[C@@H](C(=O)NCC(=O)O)[C@H](O)[C@@H](O)[C@@H]1O)C(C)C]

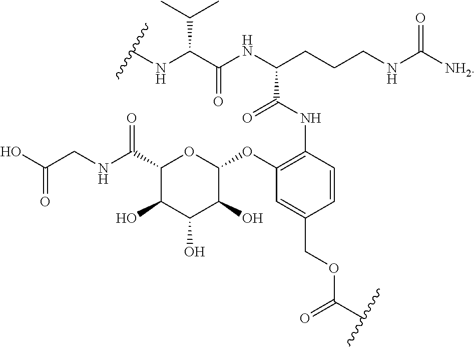 [SMILES: *N[C@@H](C(=O)N[C@H](CCCNC(N)=O)C(=O)Nc1ccc(COC(*)=O)cc1O[C@H]1O[C@@H](C(=O)NCC(=O)O)[C@H](O)[C@@H](O)[C@@H]1O)C(C)C]

In some embodiments,

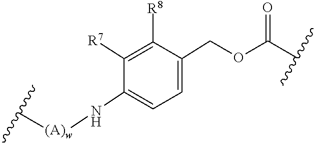

### of Formula (I) is:

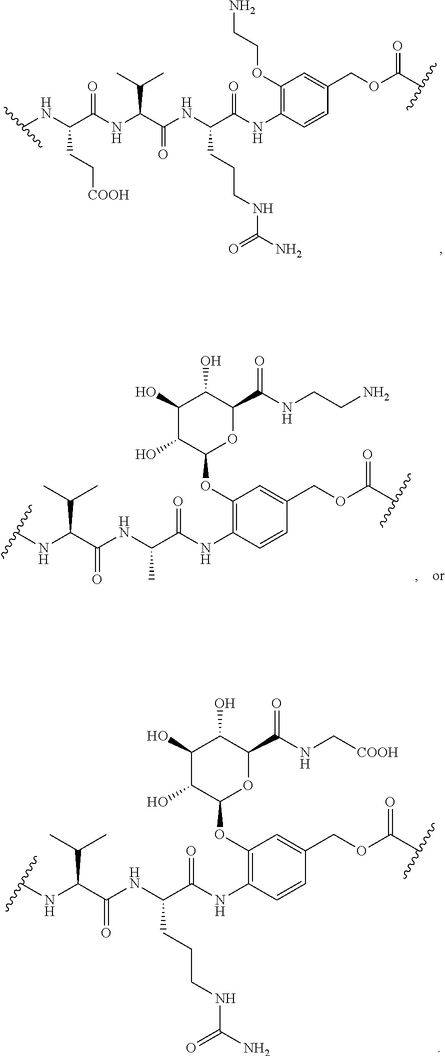 [SMILES: *N[C@@H](CCC(=O)O)C(=O)N[C@H](C(=O)N[C@@H](CCCNC(N)=O)C(=O)Nc1ccc(COC(*)=O)cc1OCCN)C(C)C ; *N[C@H](C(=O)N[C@@H](C)C(=O)Nc1ccc(COC(*)=O)cc1O[C@@H]1O[C@H](C(=O)NCCN)[C@@H](O)[C@H](O)[C@H]1O)C(C)C ; *N[C@H](C(=O)N[C@@H](CCCNC(N)=O)C(=O)Nc1ccc(COC(*)=O)cc1O[C@@H]1O[C@H](C(=O)NCC(=O)O)[C@@H](O)[C@H](O)[C@H]1O)C(C)C]

In some embodiments,

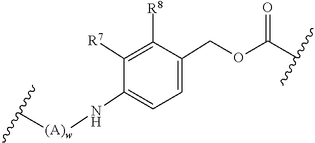

### of Formula (I) is:

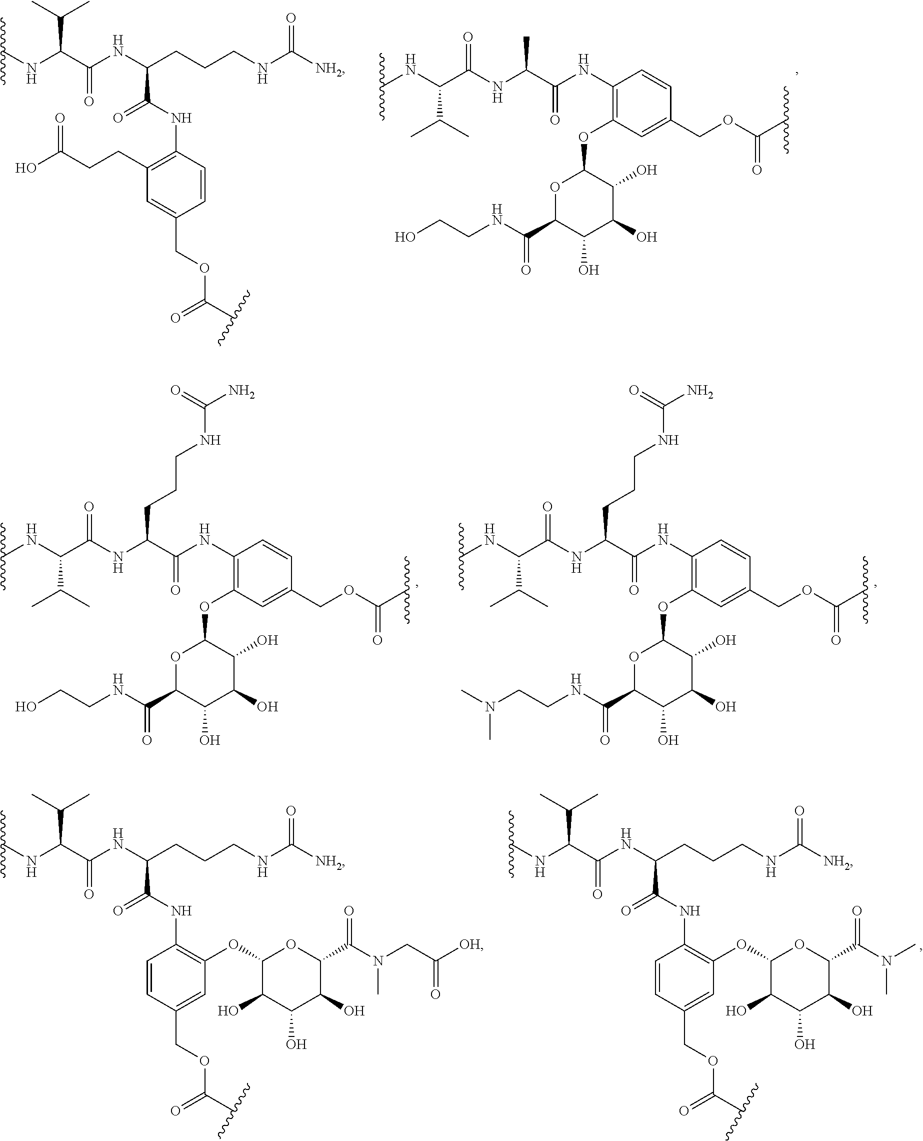

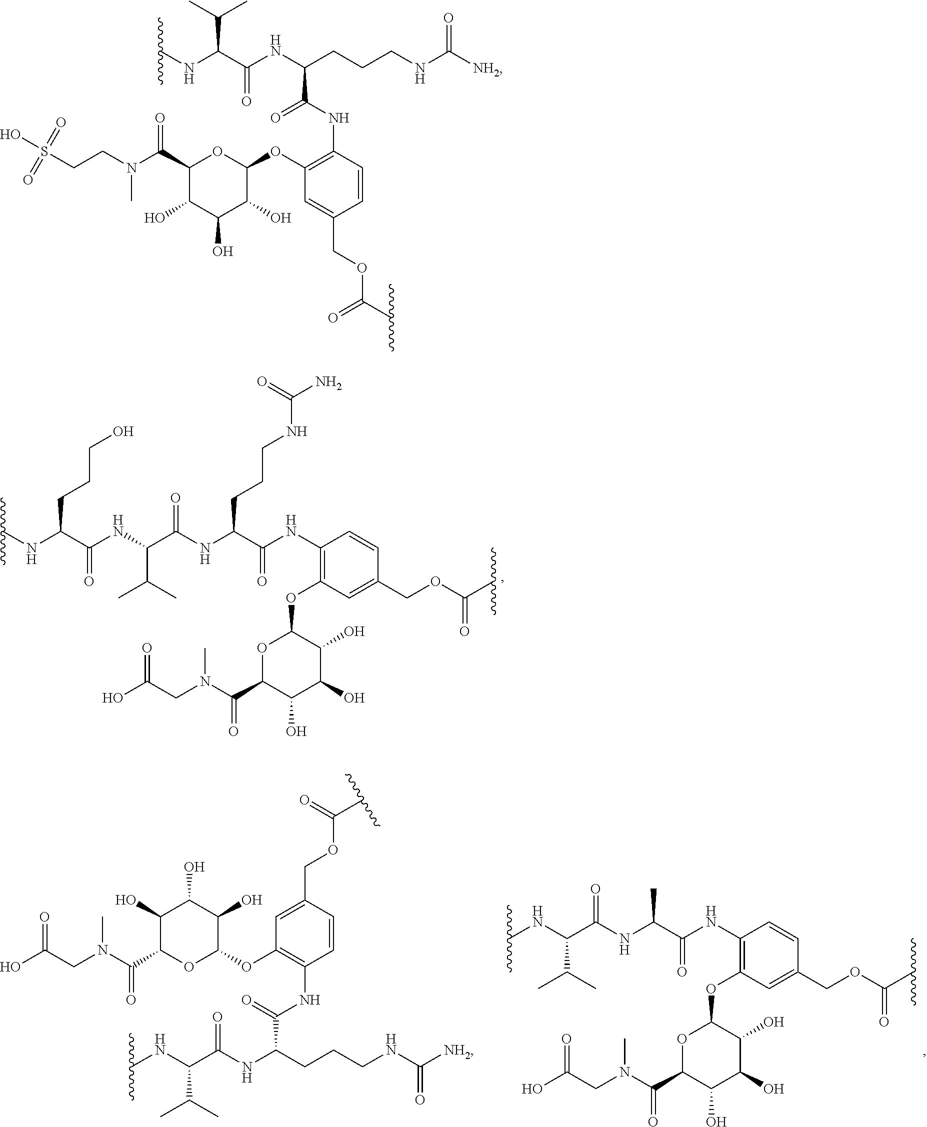 [SMILES: *N[C@H](C(=O)N[C@@H](CCCNC(N)=O)C(=O)Nc1ccc(COC(*)=O)cc1O[C@@H]1O[C@H](C(=O)N(C)CCS(=O)(=O)O)[C@@H](O)[C@H](O)[C@H]1O)C(C)C ; *N[C@@H](CCCO)C(=O)N[C@H](C(=O)N[C@@H](CCCNC(N)=O)C(=O)Nc1ccc(COC(*)=O)cc1O[C@@H]1O[C@H](C(=O)N(C)CC(=O)O)[C@@H](O)[C@H](O)[C@H]1O)C(C)C ; *N[C@H](C(=O)N[C@@H](CCCNC(N)=O)C(=O)Nc1ccc(COC(*)=O)cc1O[C@@H]1O[C@H](C(=O)N(C)CC(=O)O)[C@@H](O)[C@H](O)[C@H]1O)C(C)C ; *N[C@H](C(=O)N[C@@H](C)C(=O)Nc1ccc(COC(*)=O)cc1O[C@@H]1O[C@H](C(=O)N(C)CC(=O)O)[C@@H](O)[C@H](O)[C@H]1O)C(C)C]

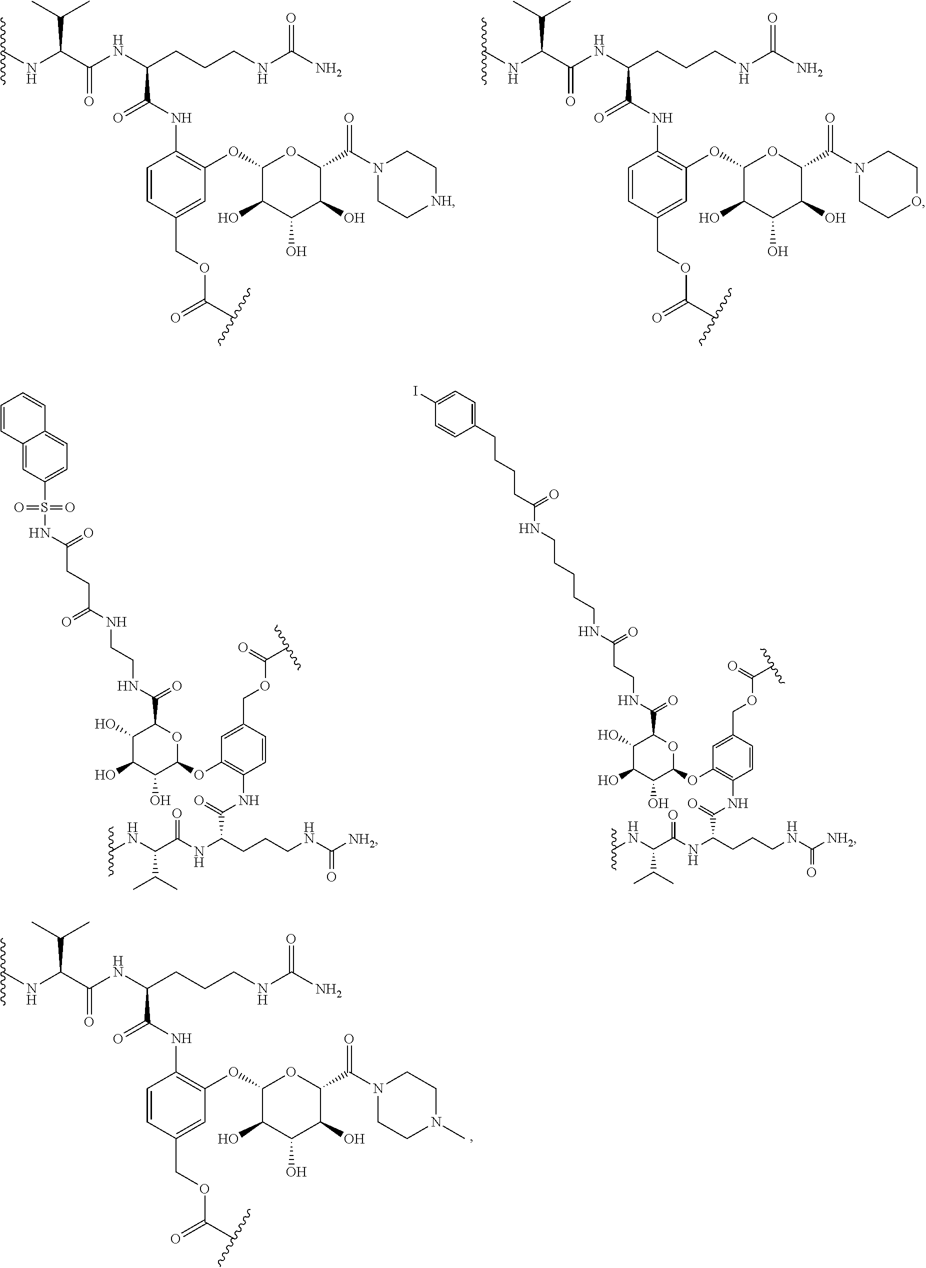

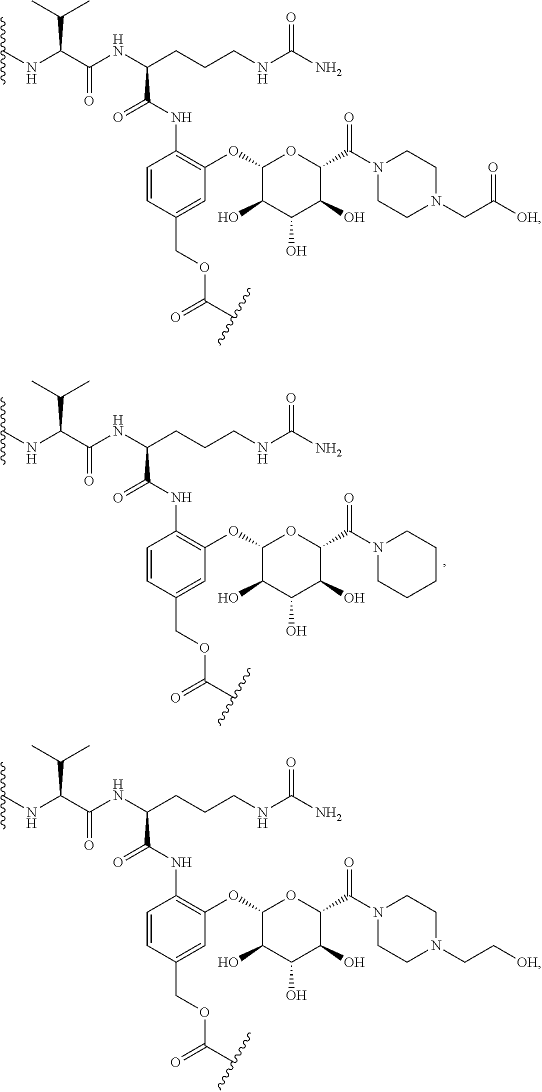 [SMILES: *N[C@H](C(=O)N[C@@H](CCCNC(N)=O)C(=O)Nc1ccc(COC(*)=O)cc1O[C@@H]1O[C@H](C(=O)N2CCN(CC(=O)O)CC2)[C@@H](O)[C@H](O)[C@H]1O)C(C)C ; *N[C@H](C(=O)N[C@@H](CCCNC(N)=O)C(=O)Nc1ccc(COC(*)=O)cc1O[C@@H]1O[C@H](C(=O)N2CCCCC2)[C@@H](O)[C@H](O)[C@H]1O)C(C)C ; *N[C@H](C(=O)N[C@@H](CCCNC(N)=O)C(=O)Nc1ccc(COC(*)=O)cc1O[C@@H]1O[C@H](C(=O)N2CCN(CCO)CC2)[C@@H](O)[C@H](O)[C@H]1O)C(C)C]

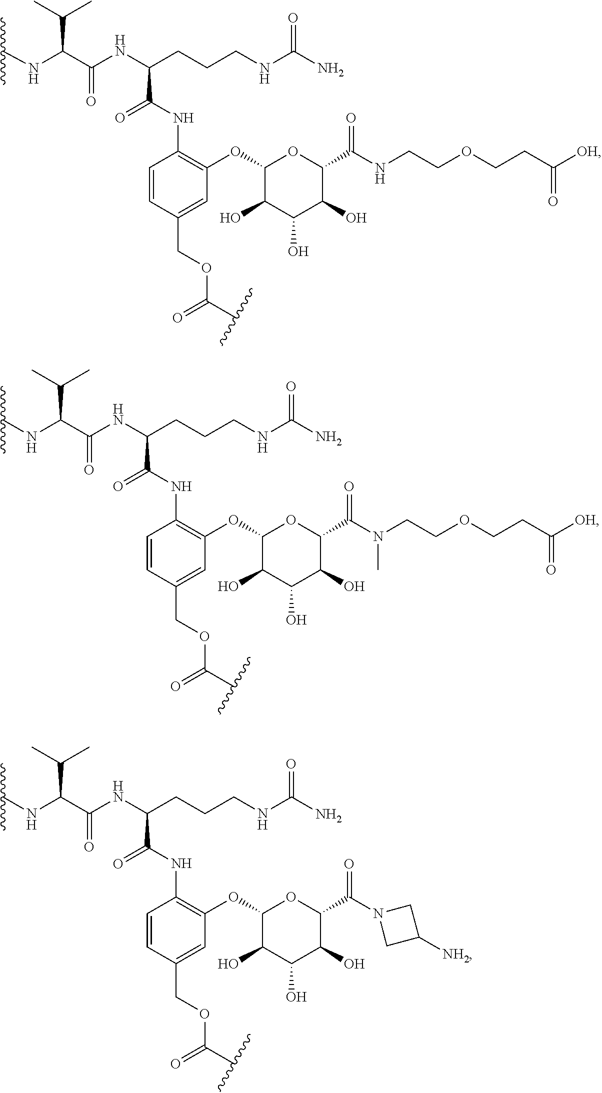 [SMILES: *N[C@H](C(=O)N[C@@H](CCCNC(N)=O)C(=O)Nc1ccc(COC(*)=O)cc1O[C@@H]1O[C@H](C(=O)NCCOCCC(=O)O)[C@@H](O)[C@H](O)[C@H]1O)C(C)C ; *N[C@H](C(=O)N[C@@H](CCCNC(N)=O)C(=O)Nc1ccc(COC(*)=O)cc1O[C@@H]1O[C@H](C(=O)N(C)CCOCCC(=O)O)[C@@H](O)[C@H](O)[C@H]1O)C(C)C ; *N[C@H](C(=O)N[C@@H](CCCNC(N)=O)C(=O)Nc1ccc(COC(*)=O)cc1O[C@@H]1O[C@H](C(=O)N2CC(N)C2)[C@@H](O)[C@H](O)[C@H]1O)C(C)C]

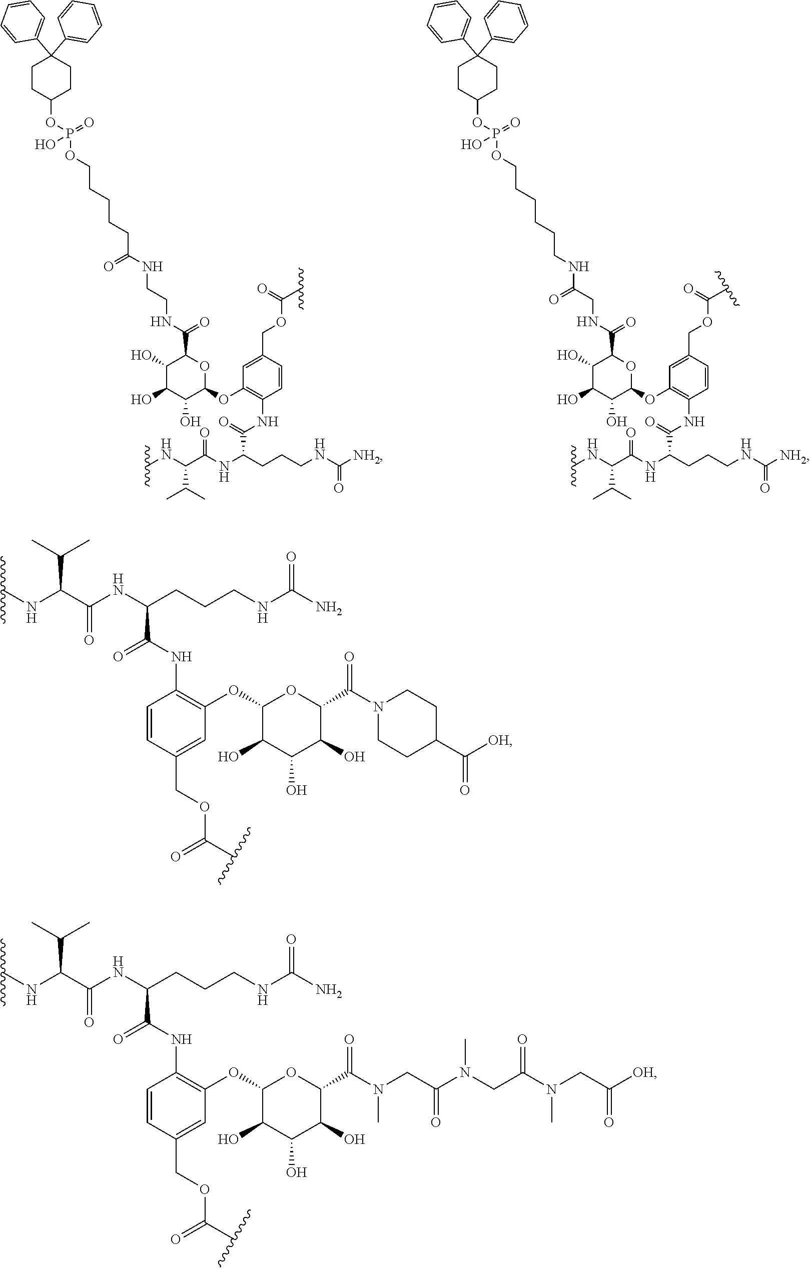

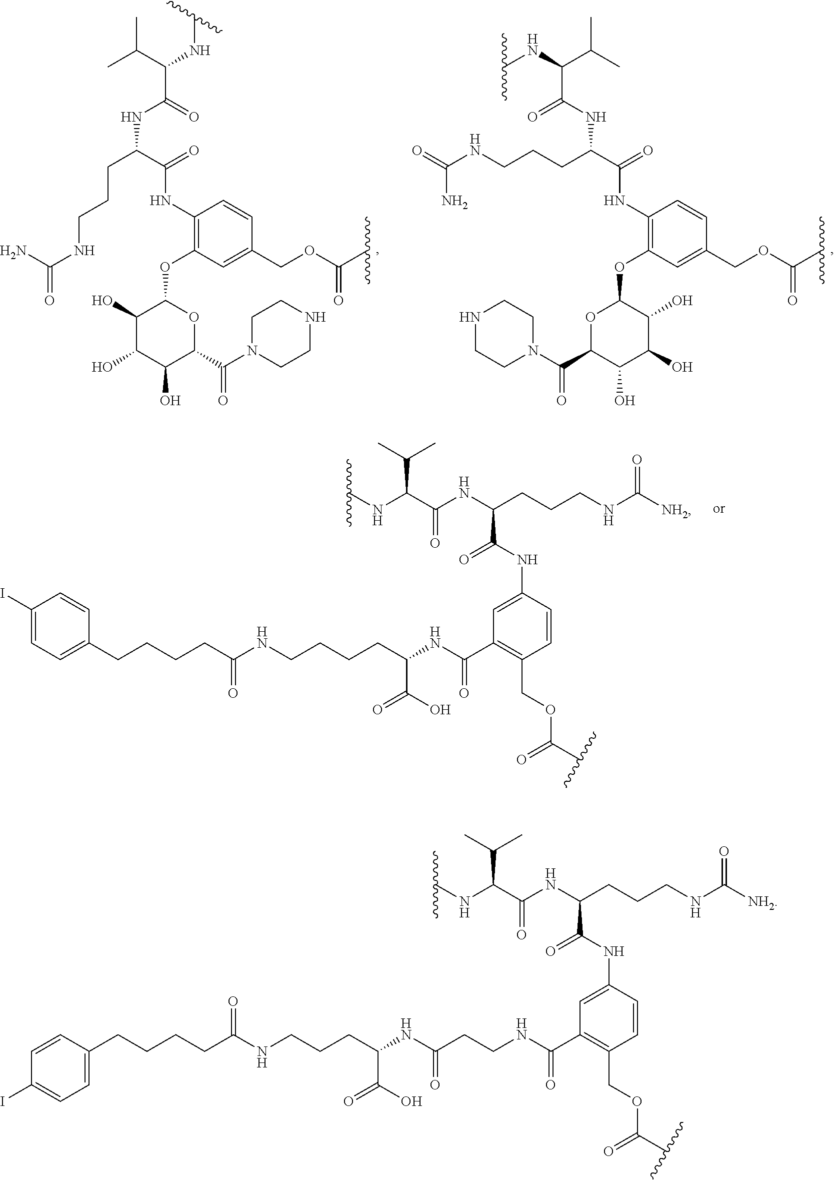 [SMILES: *N[C@H](C(=O)N[C@@H](CCCNC(N)=O)C(=O)Nc1ccc(COC(*)=O)cc1O[C@@H]1O[C@H](C(=O)N2CCNCC2)[C@@H](O)[C@H](O)[C@H]1O)C(C)C ; *N[C@H](C(=O)N[C@@H](CCCNC(N)=O)C(=O)Nc1ccc(COC(*)=O)cc1O[C@@H]1O[C@H](C(=O)N2CCNCC2)[C@@H](O)[C@H](O)[C@H]1O)C(C)C ; *N[C@H](C(=O)N[C@@H](CCCNC(N)=O)C(=O)Nc1ccc(COC(*)=O)c(C(=O)N[C@@H](CCCCNC(=O)CCCCc2ccc(I)cc2)C(=O)O)c1)C(C)C ; "or" ; *N[C@H](C(=O)N[C@@H](CCCNC(N)=O)C(=O)Nc1ccc(COC(*)=O)c(C(=O)NCCC(=O)N[C@@H](CCCNC(=O)CCCCc2ccc(I)cc2)C(=O)O)c1)C(C)C]

In some embodiments, the Spacer is

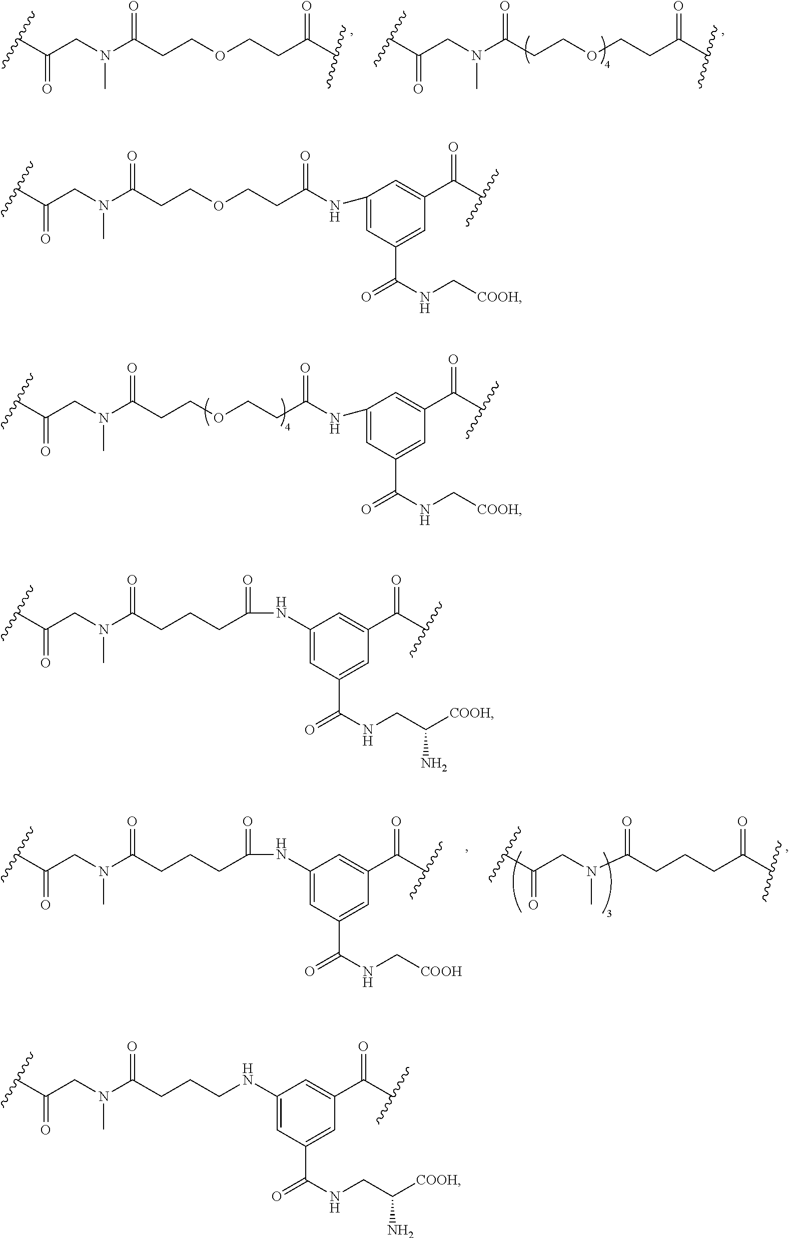

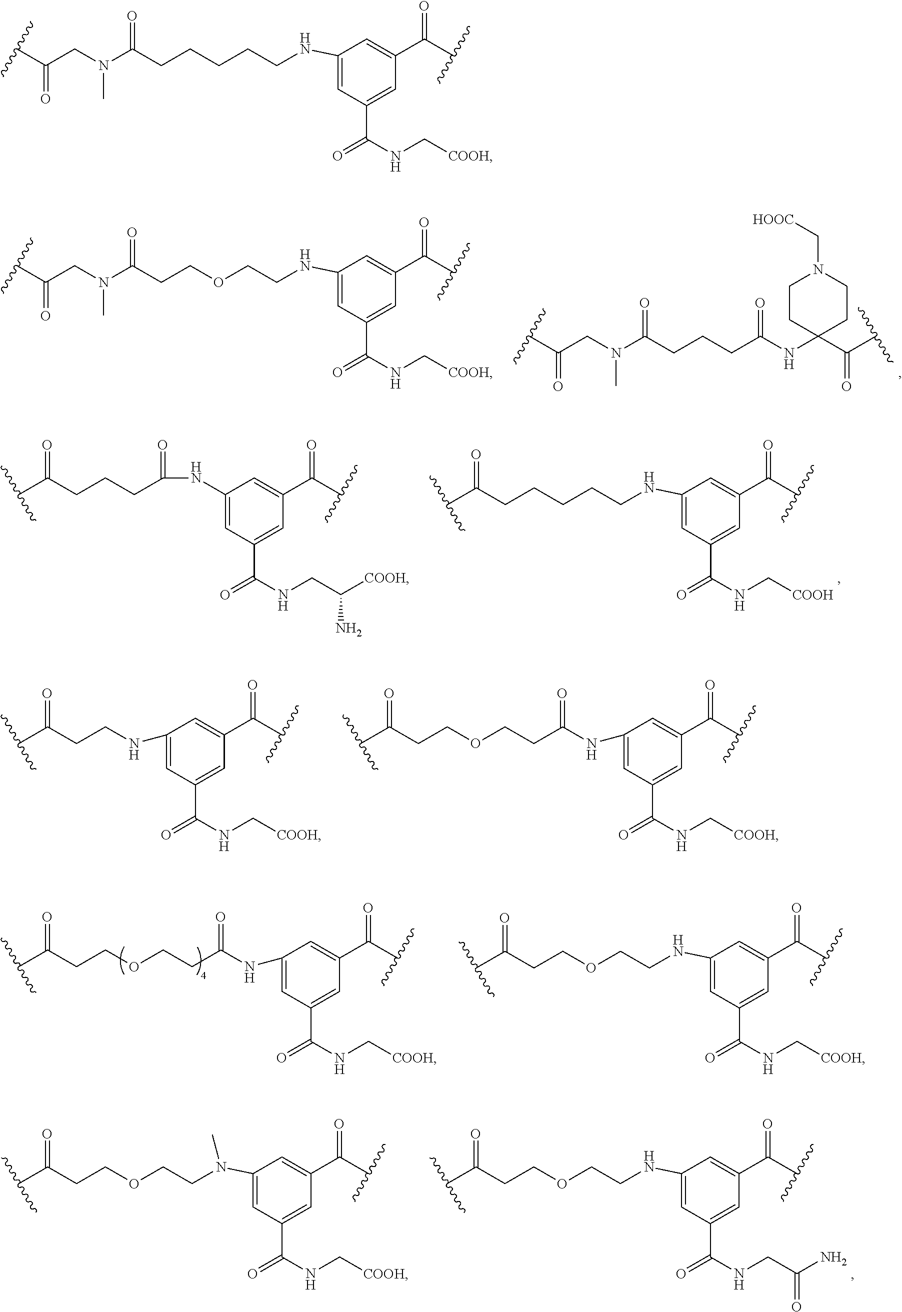

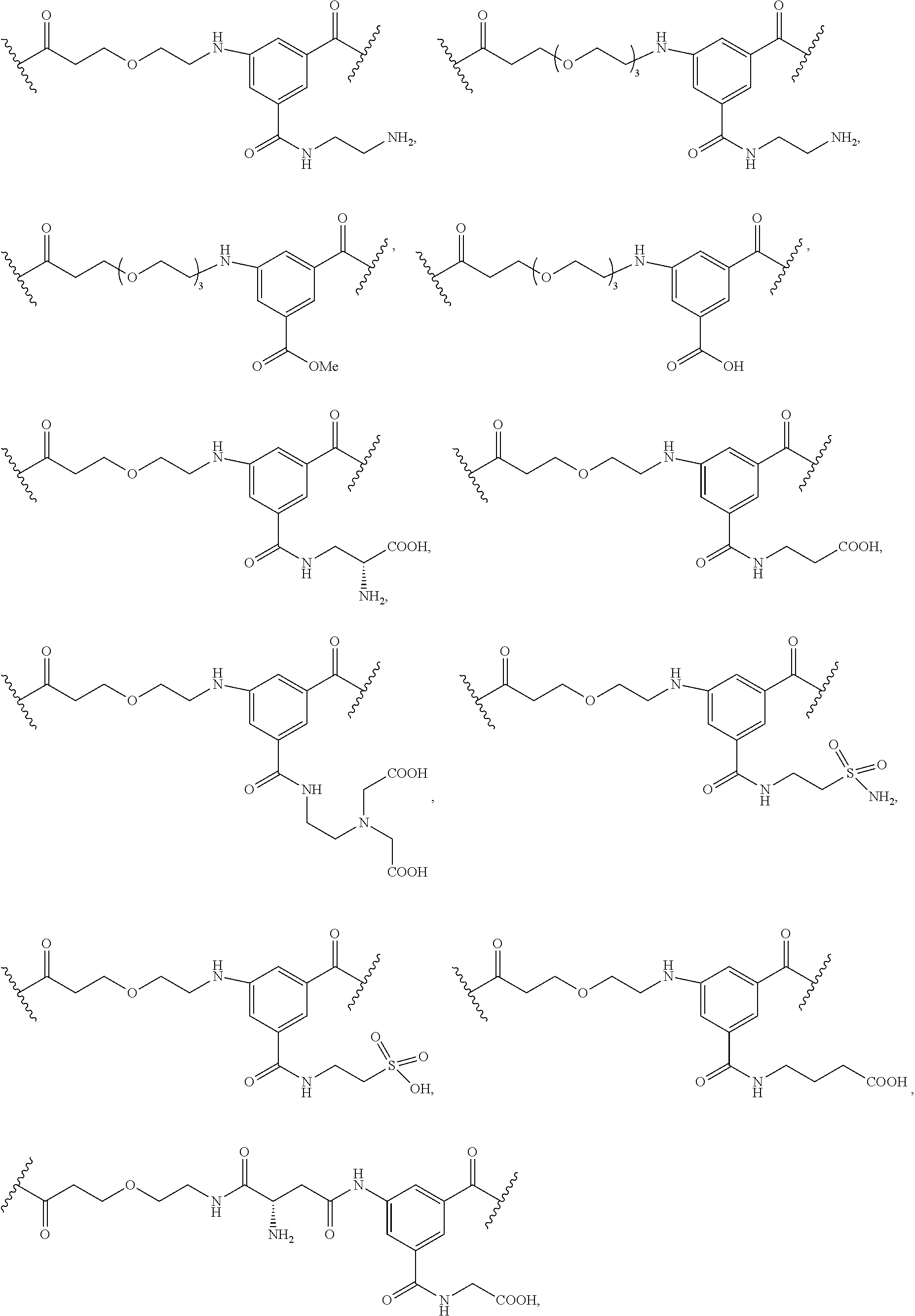

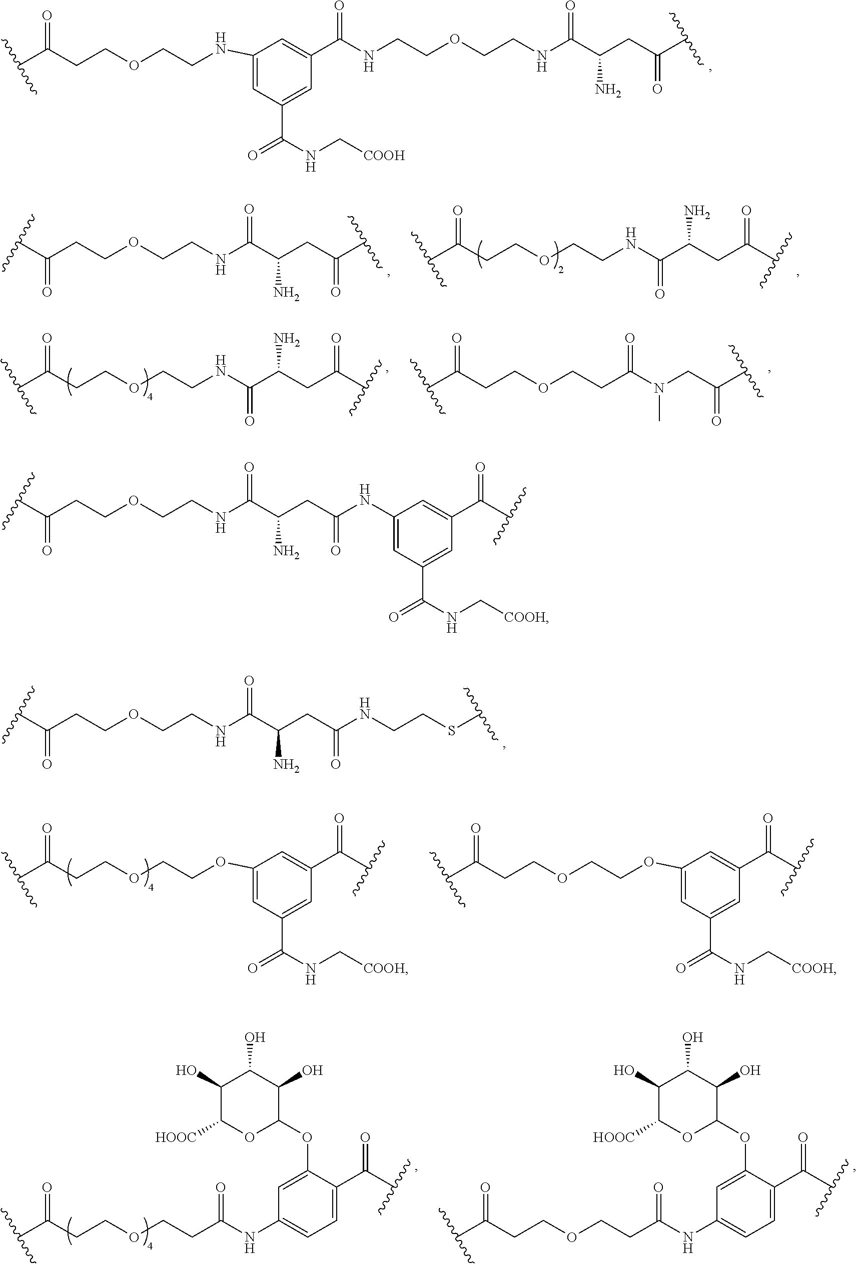

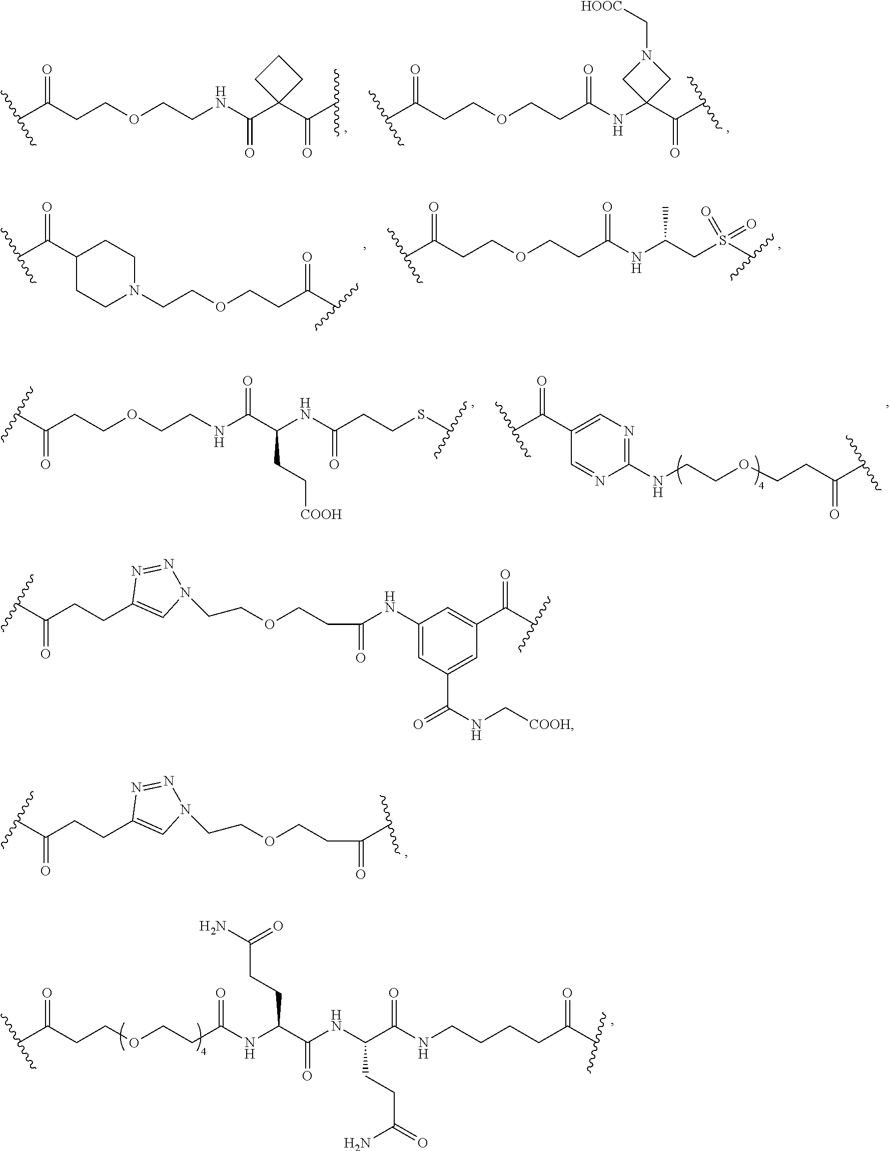

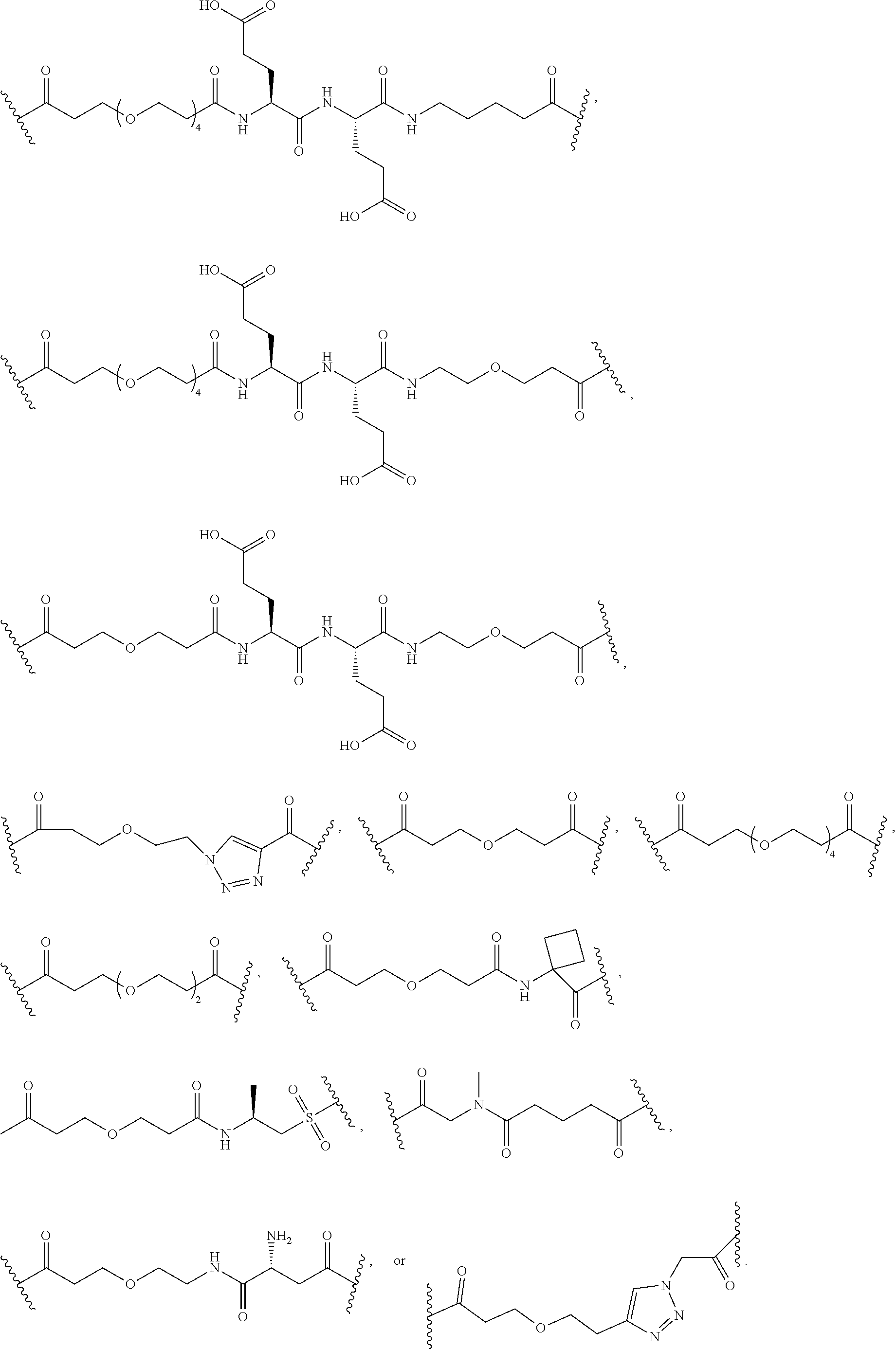

In some embodiments, the Spacer is or

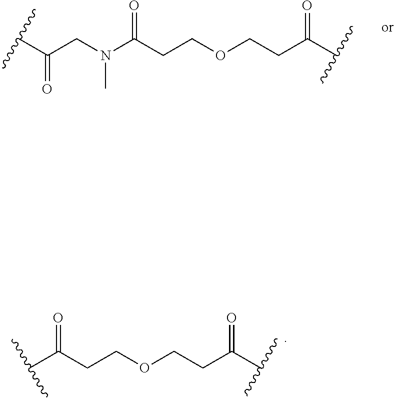 [SMILES: *C(=O)CCOCCC(=O)N(C)CC(*)=O ; *C(=O)CCOCCC(*)=O]

In some embodiments, the compound of Formula (I) is a compound of Formula (Ia):

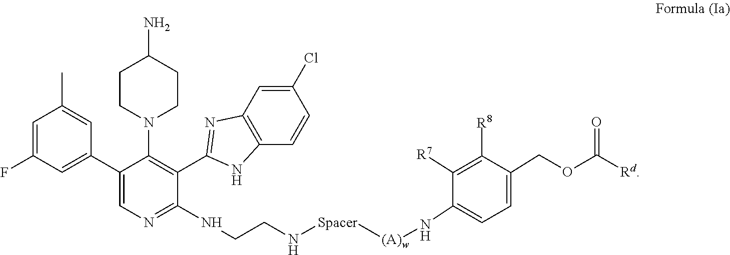

In some embodiments. R^{d}—H is monomethyl auristatin E.

In some embodiments, the compound of Formula (I) is a compound selected from Compound 1-56, 59-62, 65, 67, 69, 71-78, 83, 86-91, 198-204, 208-214, or a pharmaceutically acceptable salt thereof.

In some embodiments, the compound of Formula (I) is a compound selected from Compound 205-207, 215-238, 246, and 247, or a pharmaceutically acceptable salt thereof.

In one aspect, disclosed herein is a pharmaceutical composition comprising a compound of Formula (I), or a pharmaceutically acceptable salt thereof, and a pharmaceutically acceptable excipient.

In one aspect, disclosed herein is a method for the treatment of cancer comprising administering to a mammal with cancer an effective amount of a compound of Formula (I), or a pharmaceutically acceptable salt thereof.

In one aspect, disclosed herein is a compound that has the structure of Formula (II):

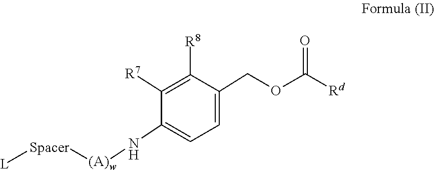

wherein:
L is a ligand;
Spacer is a spacer group;
each A is independently selected from natural or unnatural amino acids, wherein any free amine of an amino acid is optionally independently substituted with CH₃;
w is an integer selected from 1-6.
R⁷ is H, C₁-C₆ haloalkyl, OR^{7a}

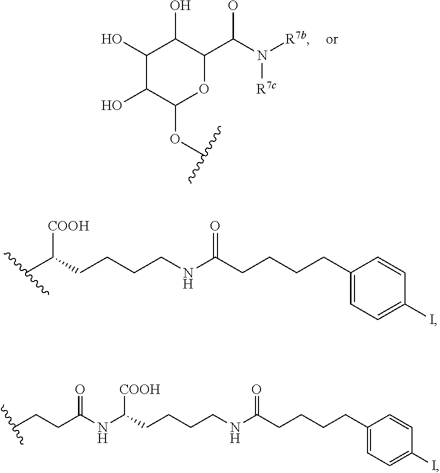

C₁-C₆ alkyl optionally substituted with COOH;
R^{7a} is H, or C₁-C₆ alkyl optionally substituted with amino, COOH, or OH,
R^{7b} is H, C₁-C₂₀ alkyl optionally substituted with OR^{7b1}, COOR^{7b1}, NR^{7b1}₂, or SO₃R^{7b1}; (CH₂)₂NHC(═O)—R^{7b2},

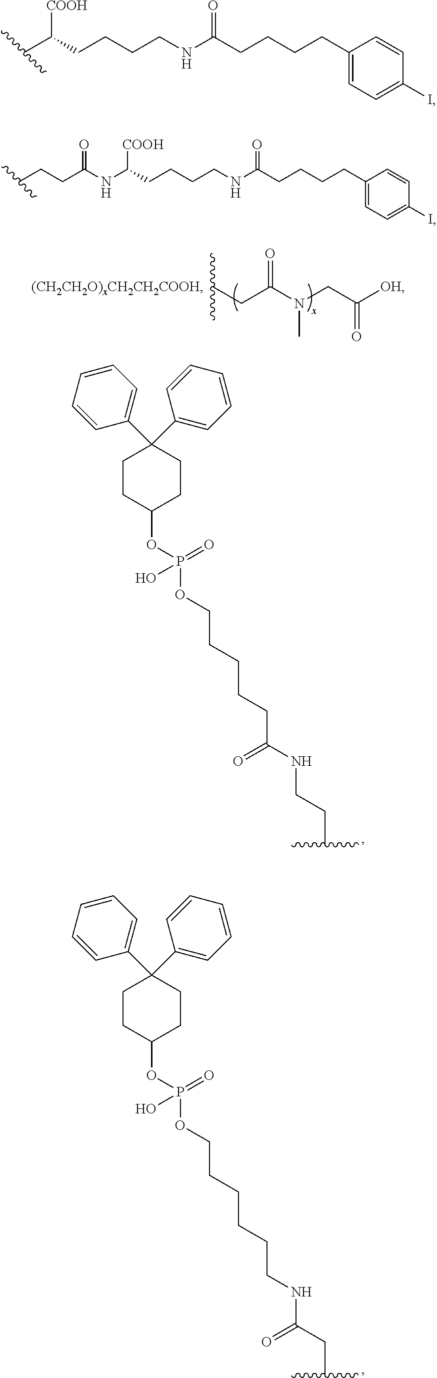

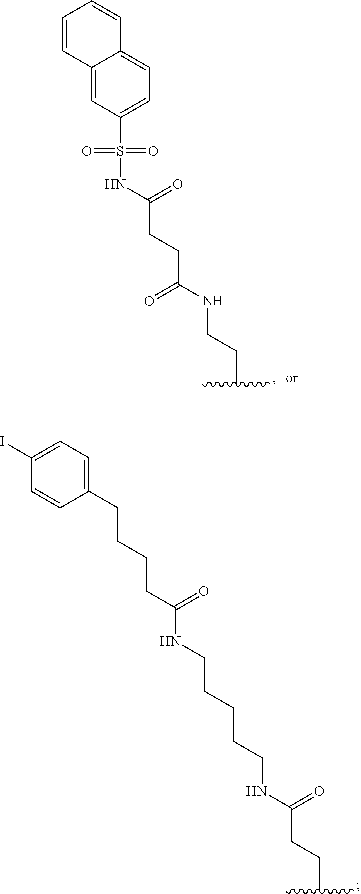 [SMILES: *CCNC(=O)CCC(=O)NS(=O)(=O)c1ccc2ccccc2c1 ; *CCC(=O)NCCCCCNC(=O)CCCCc1ccc(I)cc1]

each R^{7b1} is independently selected from H and C₁-C₆ alkyl;
R^{7b2} is (CH₂CH₂O)ₓCH₂CH₂COOH, or (CH₂)_{y}COOH;
R^{7c} is H or C₁-C₆ alkyl; or
R^{7b} and R^{7c}, together with the nitrogen to which they are attached, combine to form a heterocycloalkyl group optionally substituted with amino, COOH, or C₁-C₅ alkyl that is optionally substituted with OH or COOH;
R⁸ is H, C₁-C₆ haloalkyl, COOH, C(═O)NH—R^{8a},

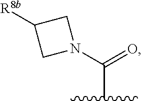

or C(═O)NH(CH₂)₂NHC(═O)—R^{8c};
R^{8a} is C₁-C₂₀ alkyl substituted with COOH or SO₃H, or C₁-C₆ alkoxy optionally substituted with amino;
R^{8b} is COOH or amino;
R^{8c} is (CH₂CH₂O)ₓCH₂CH₂COOH or (CH₂)_{y}COOH;
each x is independently an integer selected from 1-10;
each y is independently an integer selected from 1-20;
R^{d} is the residue of a bioactive molecule of the formula R^{d}—H;
wherein when R⁷ is H, then R⁸ is C₁-C₆ haloalkyl, COOH, C(═O)NH—R^{8a},

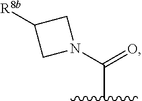

or C(═O)NH(CH₂)₂NHC(═O)—R^{8c}; and
wherein when R⁸ is H, then R⁷ is C₁-C₆ haloalkyl, OR^{7a},

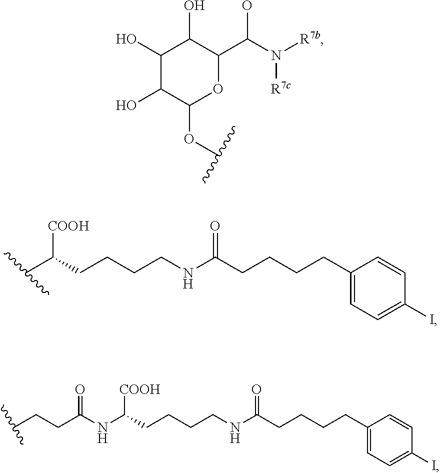

or C₁-C₆ alkyl optionally substituted with COOH.

In some embodiments, R⁷ is H, C₁-C₃ haloalkyl, OR^{7a},

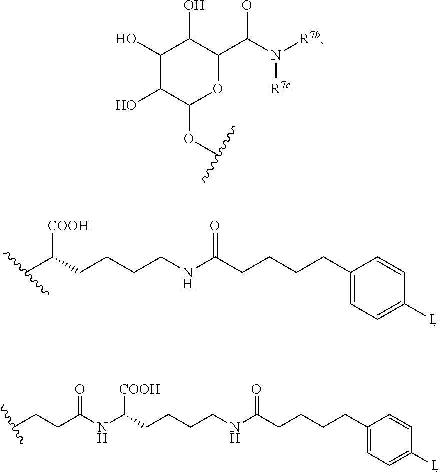

or C₁-C₃ alkyl optionally substituted with COOH;
R^{7a} is H, or C₁-C₃ alkyl optionally substituted with amino, COOH, or OH, H, C₁-C₁₄ alkyl optionally substituted with OR^{7b1}, COOR^{7b1}, NR^{7b1}₂, or SO₃R_{7b1}; (CH₂)₂NHC(═O)—R^{7b2},

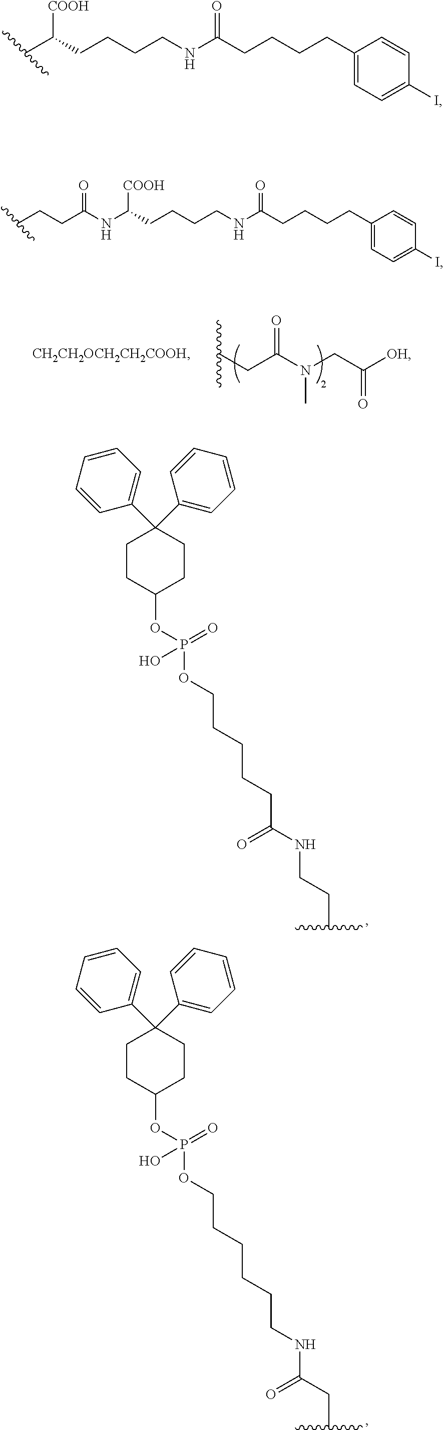

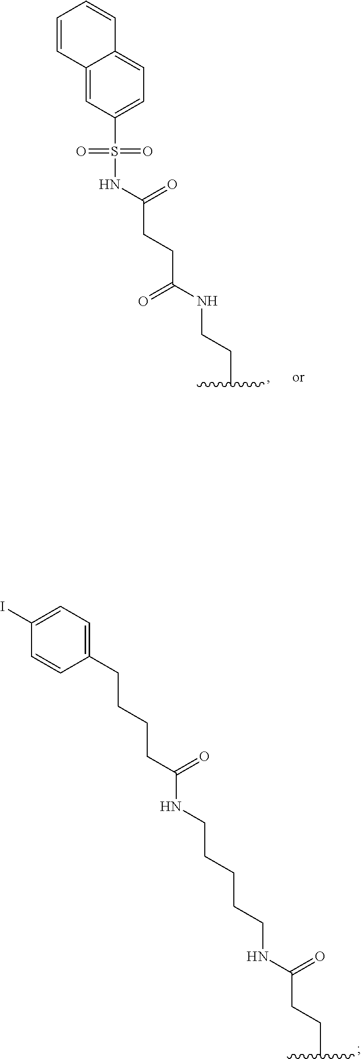 [SMILES: *CCNC(=O)CCC(=O)NS(=O)(=O)c1ccc2ccccc2c1 ; *CCC(=O)NCCCCCNC(=O)CCCCc1ccc(I)cc1]

R^{7b1} is H or C₁-C₃ alkyl;
R^{7b2} is (CH₂CH₂O)ₓCH₂CH₂COOH or (CH₂)_{y}COOH;
R^{7c} is H or C₁-C₃ alkyl; or
R^{7b} and R^{7c}, together with the nitrogen to which they are attached, combine to form a 3-6-membered heterocycloalkyl group optionally substituted with amino, COOH, or C₁-C₃ alkyl that is optionally substituted with OH or COOH;
R⁸ is H, C₁-C₃ haloalkyl, COOH, C(═O)NH—R^{8a},

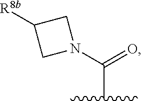

or C(═O)NH(CH₂)₂NHC(═O)—R^{8c};
R^{8a} is C₁-C₁₀ alkyl substituted with COOH or SO₃H, or C₁-C₃ alkoxy optionally substituted with amino;
R^{8b} is COOH or amino;
R^{8c} is (CH₂CH₂O)ₓCH₂CH₂COOH or (CH₂)_{y}COOH;
wherein when R⁷ is H, then R⁸ is C₁-C₃ haloalkyl, COOH, C(═O)NH—R^{8a},

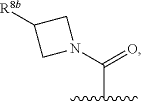

or C(═O)NH(CH₂)₂NHC(═O)—R^{8c}; and
wherein when R⁸ is H, then R⁷ is C₁-C₃ haloalkyl, OR^{7a},

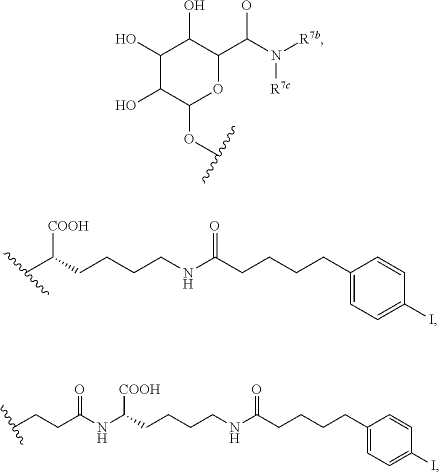

or C₁-C₃ alkyl optionally substituted with COOH.

In some embodiments,
L is a ligand;
Spacer is a spacer group;
each A is independently selected from natural or unnatural amino acids, wherein any free amine of an amino acid is optionally independently substituted with CH₃;
w is an integer selected from 1-6;
R⁷ is H, C₁-C₆ alkyl, C₁-C₆ haloalkyl, OR^{7a}, or

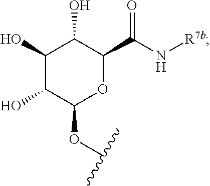

R^{7a} is H; or C₁-C₆ alkyl optionally substituted with amino, COOH, or OH;
R^{7b} is H; C₁-C₂₀ alkyl substituted with COOR^{7b1} amino or SO₃H; CH₂)NHC(═O)—R^{7b2}:

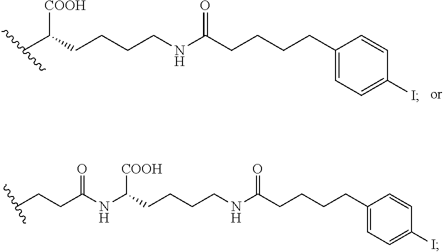 [SMILES: *[C@@H](CCCCNC(=O)CCCCc1ccc(I)cc1)C(=O)O ; "or" ; *CCC(=O)N[C@@H](CCCCNC(=O)CCCCc1ccc(I)cc1)C(=O)O]

R^{7b1} is H or C₁-C₆ alkyl;
R^{7b2} is (CH₂CH₂O)ₓCH₂CH₂COOH, or (CH₂)_{y}COOH;
R⁸ is H, C₁-C₆ haloalkyl, COOH, C(═O)NH—R^{8a},

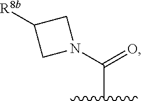

or C(═O)NH(CH₂)₂NHC(═O)—R^{8c};
R^{8a} is C₁-C₂₀ alkyl substituted with COOH or SO₃H; or C₁-C₆ alkoxy optionally substituted with amino;
R^{8b} is COOH or amino;
R^{8c} is (CH₂CH₂O)ₓCH₂CH₂COOH or (CH₂)_{y}COOH;
each x is independently an integer selected from 1-10;
each y is independently an integer selected from 1-20;
R^{d} is the residue of a bioactive molecule of the formula R^{d}—H;
wherein when R⁷ is H, then R⁸ is C₁-C₆ haloalkyl, COOH, C(═O)NH—R^{8a},

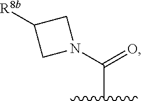

or C(═O)NH(CH₂)₂NHC(═O)—R^{8c}; and
wherein when R⁸ is H, then R⁷ is C₁-C₆ alkyl, C₁-C₆ haloalkyl, OR^{7a}, or

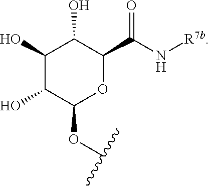

In some embodiments. R⁷ is H, C₁-C₃ alkyl, C₁-C₃ haloalkyl, OR^{7a}, or

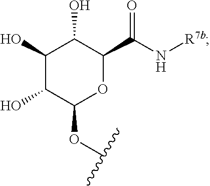

R^{7a} is H; or C₁-C₃ alkyl optionally substituted with amino, COOH, or OH;
R^{7b} is H; C₁-C₁₄ alkyl substituted with COOR^{7b1}, amino, or SO₃H; (CH₂)₂NHC(═O)—R^{7b2};

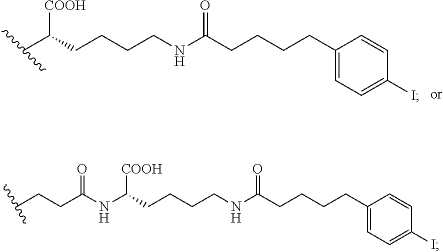 [SMILES: *[C@@H](CCCCNC(=O)CCCCc1ccc(I)cc1)C(=O)O ; "or" ; *CCC(=O)N[C@@H](CCCCNC(=O)CCCCc1ccc(I)cc1)C(=O)O]

R^{7b1} is H or C₁-C₃ alkyl;
R^{7b2} is (CH₂CH₂O)ₓCH₂CH₂COOH, or (CH₂)_{y}COOH;
R⁸ is H, C₁-C₃ haloalkyl, COOH, C(═O)NH—R^{8a},

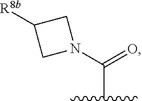

or C(═O)NH(CH₂)₂NHC(═O)—R^{8c};
R^{8a} is C₁-C₁₀ alkyl substituted with COOH or SO₃H; or C₁-C₃ alkoxy optionally substituted with amino;
R^{8b} is COOH or amino,
R^{8c} is (CH₂CH₂O)ₓCH₂CH₂COOH or (CH₂)_{y}COOH;
wherein when R⁷ is H, then R⁸ is C₁-C₃ haloalkyl, COOH, C(═O)NH—R^{8a},

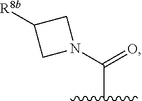

or C(═O)NH(CH₂)₂NHC(═O)—R^{8c}; and
wherein when R⁸ is H, then R⁷ is C₁-C₃ alkyl, C₁-C₃ haloalkyl, OR^{7a}, or

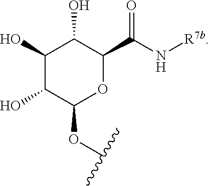

In some embodiments, R⁷ is methyl, trifluoromethyl, OR^{7a}, or

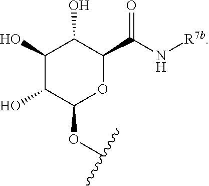

In some embodiments, R⁷ is OR^{7a} or

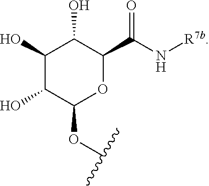

In some embodiments. R⁷ is

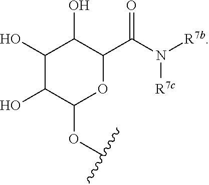

In some embodiments, R⁸ is trifluoromethyl, COOH, C(═O)NH—R^{8a},

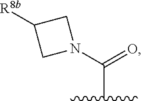

or C(═O)NH(CH₂)₂NHC(═O)—R^{8c}.

In some embodiments, R⁸ is C(═O)NH—R^{8a},

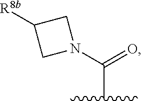

or C(═O)NH(CH₂)₂NHC(═O)—R^{8c}.

In some embodiments.

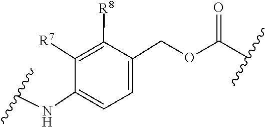

### of Formula (II) is:

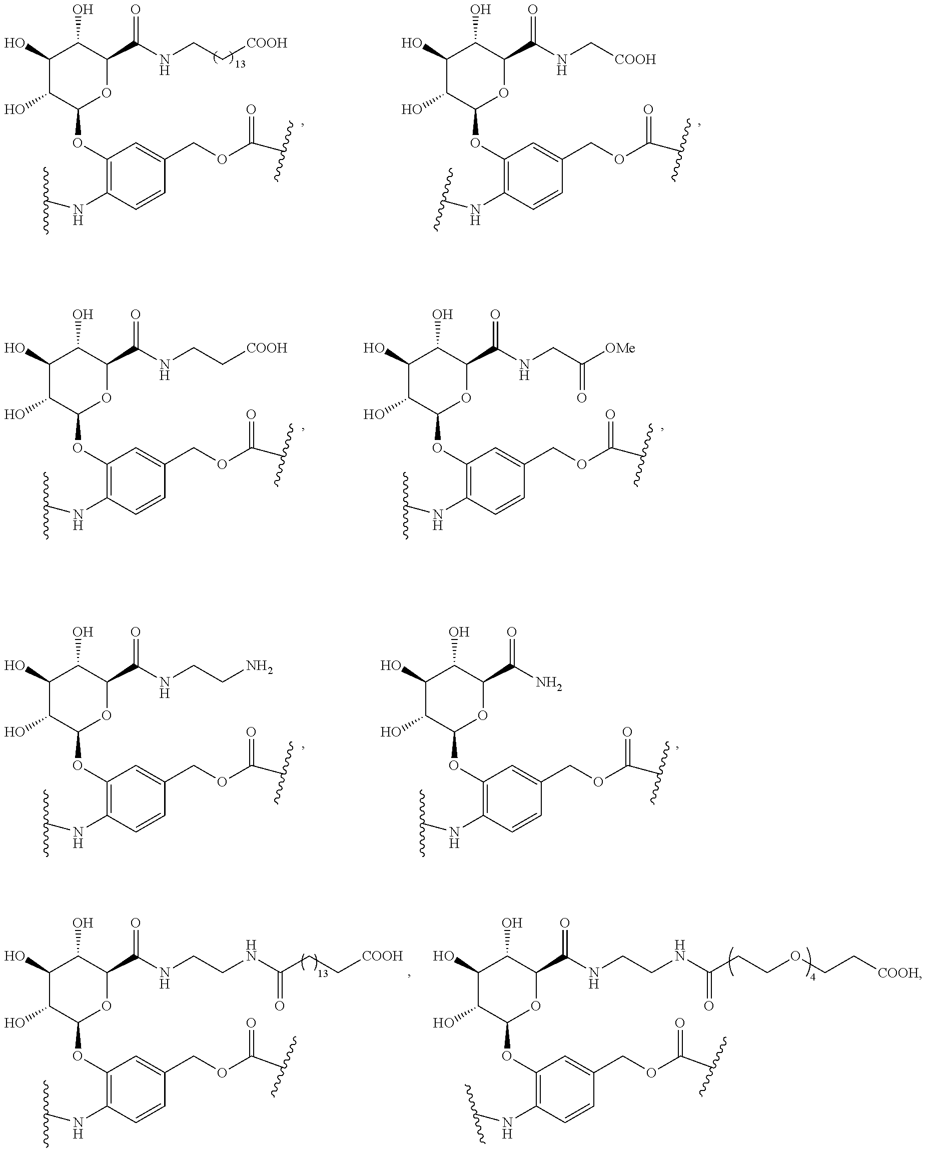

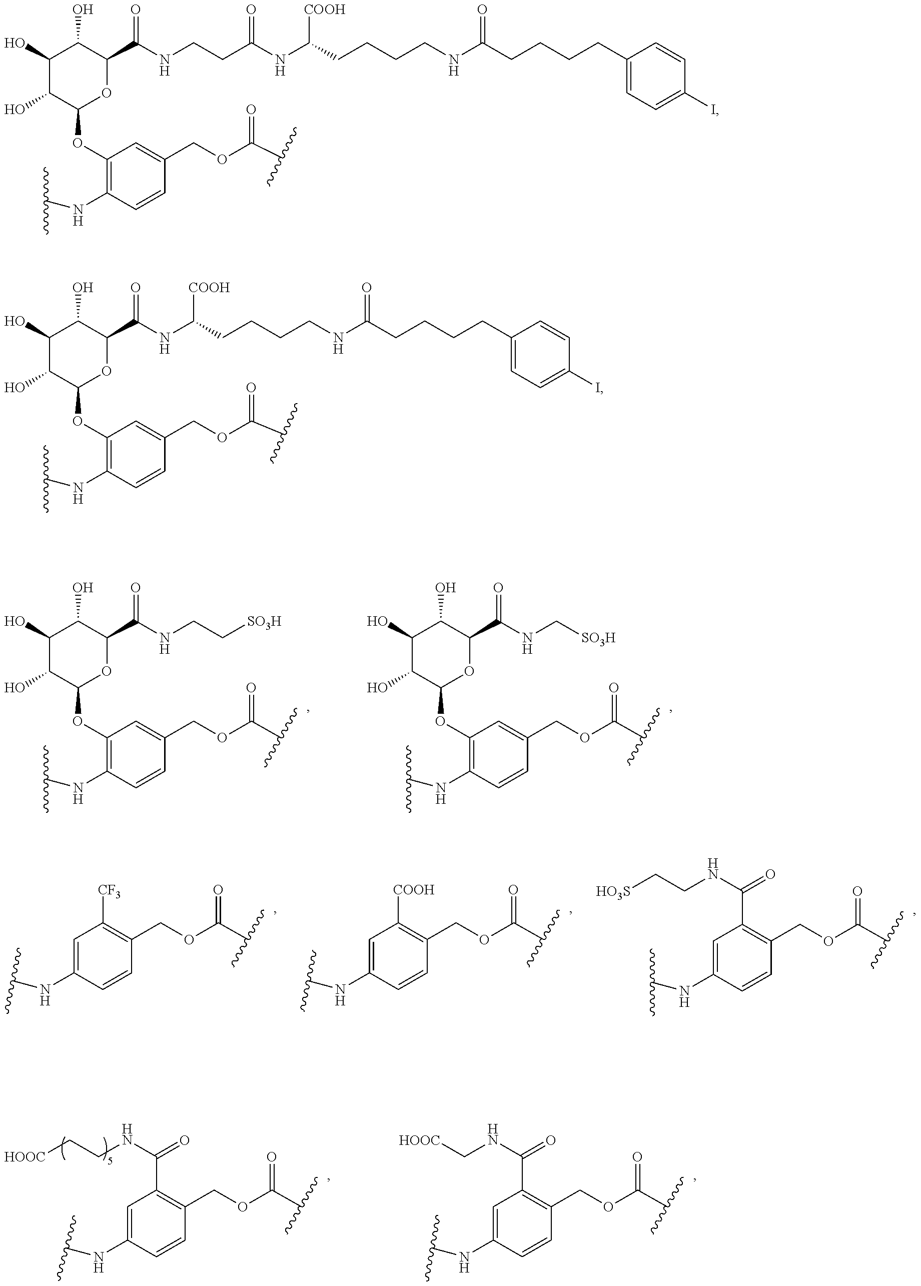

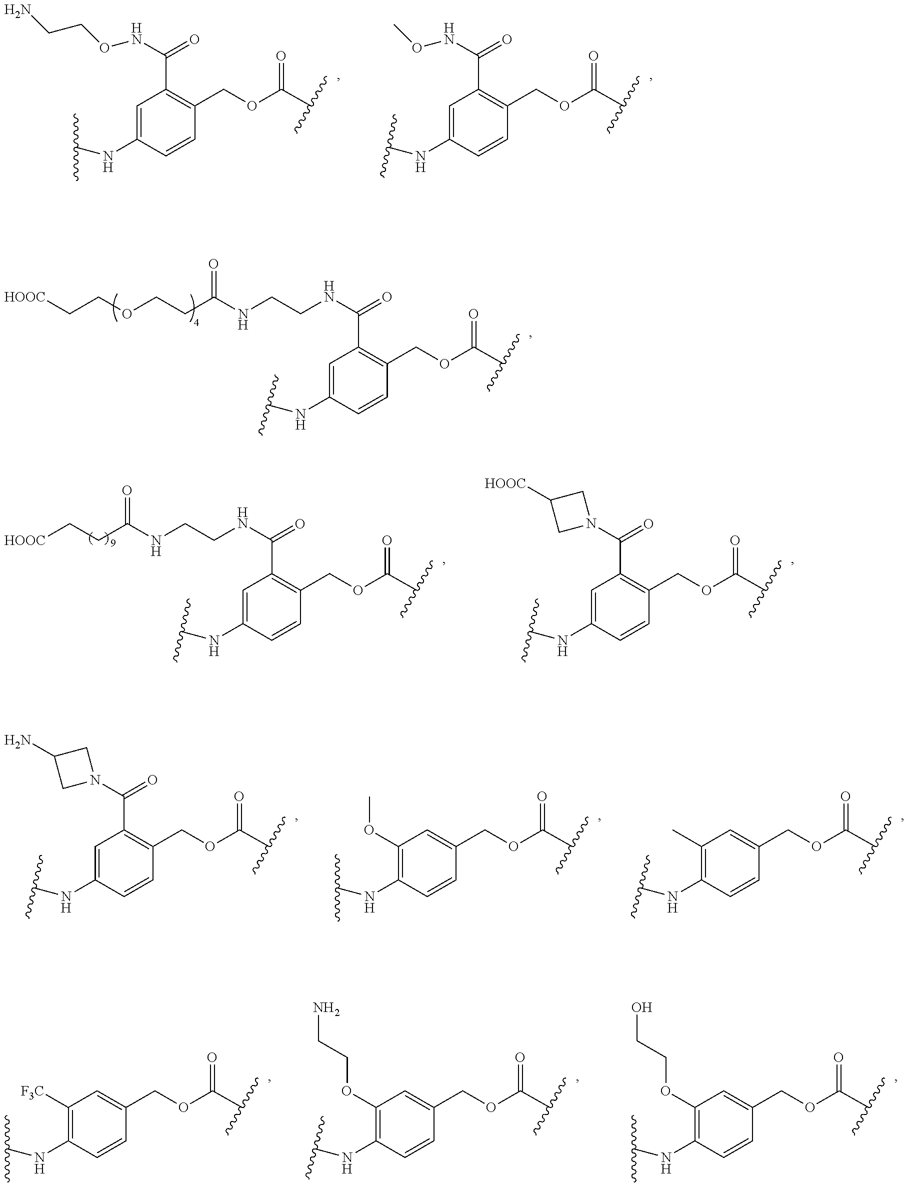

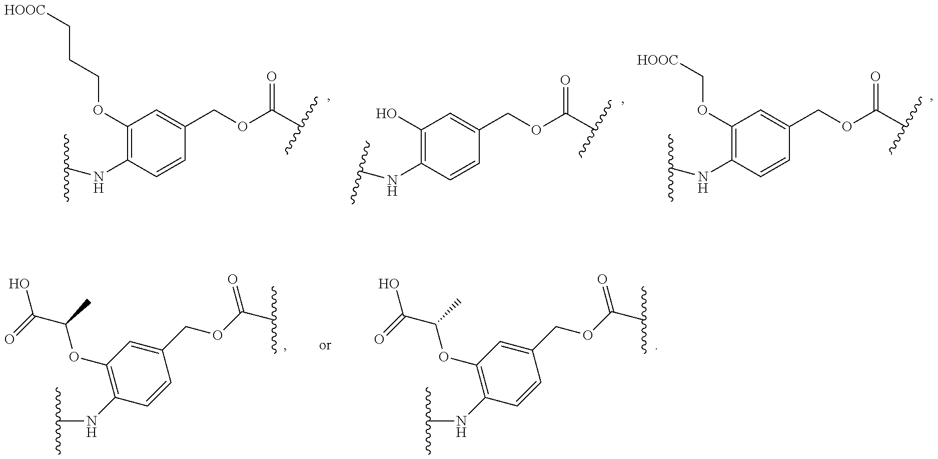 [SMILES: *Nc1ccc(COC(*)=O)cc1OCCCC(=O)O ; *Nc1ccc(COC(*)=O)cc1O ; *Nc1ccc(COC(*)=O)cc1OCC(=O)O ; *Nc1ccc(COC(*)=O)cc1O[C@H](C)C(=O)O ; *Nc1ccc(COC(*)=O)cc1O[C@@H](C)C(=O)O]

In some embodiments,

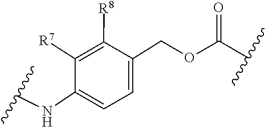

### Formula (II) is:

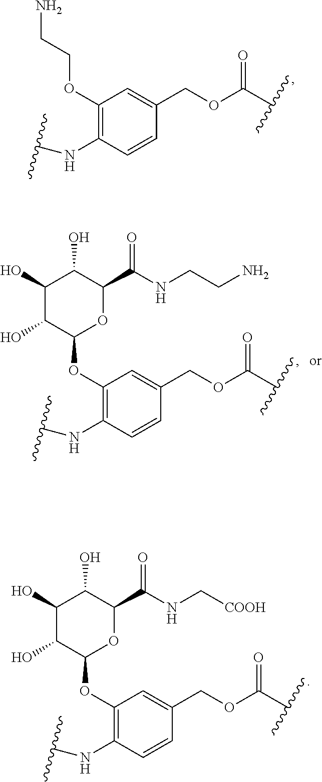 [SMILES: *Nc1ccc(COC(*)=O)cc1OCCN ; *Nc1ccc(COC(*)=O)cc1O[C@@H]1O[C@H](C(=O)NCCN)[C@@H](O)[C@H](O)[C@H]1O ; *Nc1ccc(COC(*)=O)cc1O[C@@H]1O[C@H](C(=O)NCC(=O)O)[C@@H](O)[C@H](O)[C@H]1O]

In some embodiments,

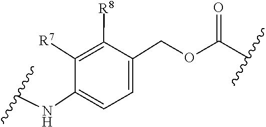

### of Formula (II) is:

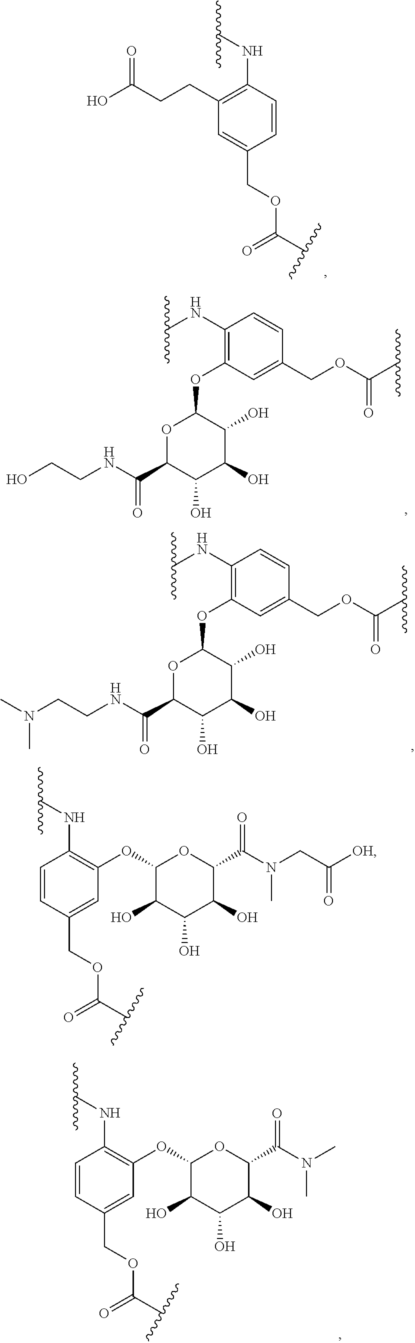

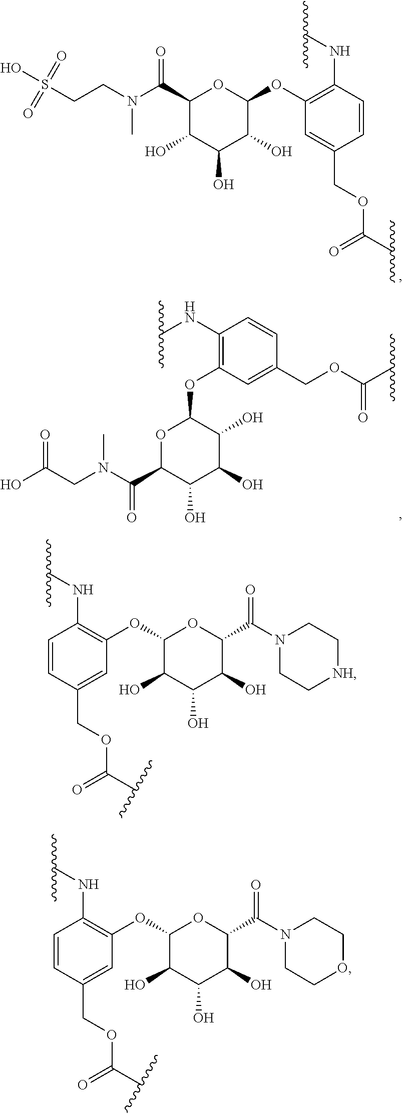 [SMILES: *Nc1ccc(COC(*)=O)cc1O[C@@H]1O[C@H](C(=O)N(C)CCS(=O)(=O)O)[C@@H](O)[C@H](O)[C@H]1O ; *Nc1ccc(COC(*)=O)cc1O[C@@H]1O[C@H](C(=O)N(C)CC(=O)O)[C@@H](O)[C@H](O)[C@H]1O ; *Nc1ccc(COC(*)=O)cc1O[C@@H]1O[C@H](C(=O)N2CCNCC2)[C@@H](O)[C@H](O)[C@H]1O ; *Nc1ccc(COC(*)=O)cc1O[C@@H]1O[C@H](C(=O)N2CCOCC2)[C@@H](O)[C@H](O)[C@H]1O]

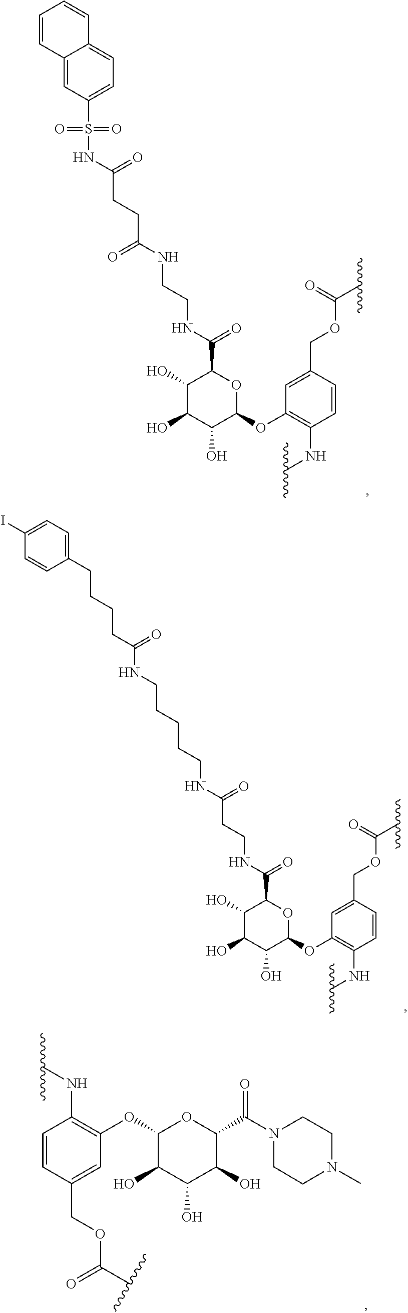 [SMILES: *Nc1ccc(COC(*)=O)cc1O[C@@H]1O[C@H](C(=O)NCCNC(=O)CCC(=O)NS(=O)(=O)c2ccc3ccccc3c2)[C@@H](O)[C@H](O)[C@H]1O ; *Nc1ccc(COC(*)=O)cc1O[C@@H]1O[C@H](C(=O)NCCC(=O)NCCCCCNC(=O)CCCCc2ccc(I)cc2)[C@@H](O)[C@H](O)[C@H]1O ; *Nc1ccc(COC(*)=O)cc1O[C@@H]1O[C@H](C(=O)N2CCN(C)CC2)[C@@H](O)[C@H](O)[C@H]1O]

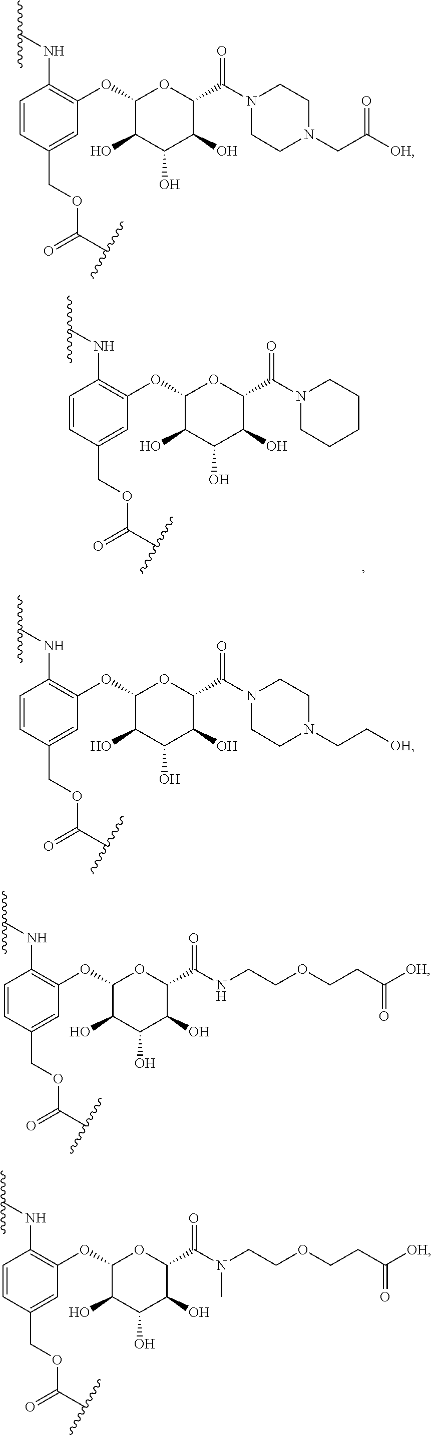 [SMILES: *Nc1ccc(COC(*)=O)cc1O[C@@H]1O[C@H](C(=O)N2CCN(CC(=O)O)CC2)[C@@H](O)[C@H](O)[C@H]1O ; *Nc1ccc(COC(*)=O)cc1O[C@@H]1O[C@H](C(=O)N2CCCCC2)[C@@H](O)[C@H](O)[C@H]1O ; *Nc1ccc(COC(*)=O)cc1O[C@@H]1O[C@H](C(=O)N2CCN(CCO)CC2)[C@@H](O)[C@H](O)[C@H]1O ; *Nc1ccc(COC(*)=O)cc1O[C@@H]1O[C@H](C(=O)NCCOCCC(=O)O)[C@@H](O)[C@H](O)[C@H]1O ; *Nc1ccc(COC(*)=O)cc1O[C@@H]1O[C@H](C(=O)N(C)CCOCCC(=O)O)[C@@H](O)[C@H](O)[C@H]1O]

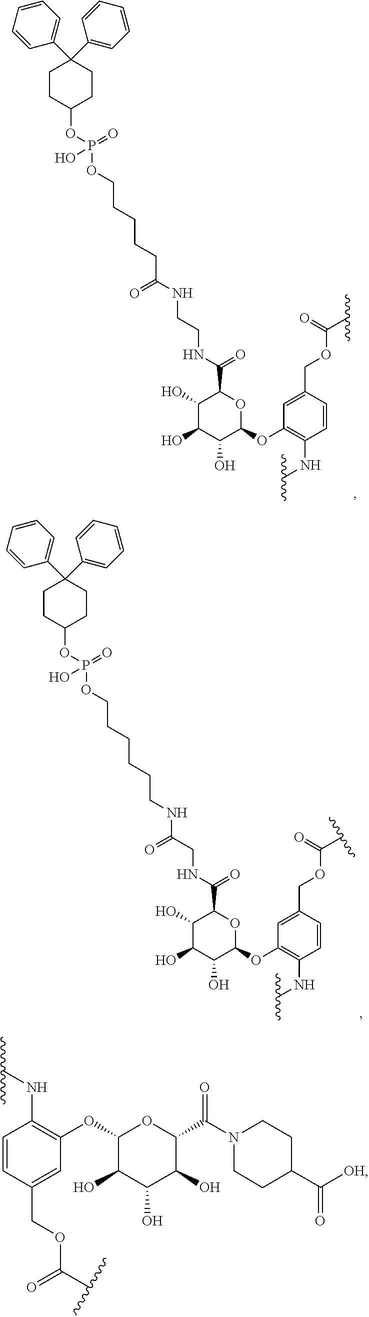 [SMILES: *Nc1ccc(COC(*)=O)cc1O[C@@H]1O[C@H](C(=O)NCCNC(=O)CCCCCOP(=O)(O)OC2CCC(c3ccccc3)(c3ccccc3)CC2)[C@@H](O)[C@H](O)[C@H]1O ; *Nc1ccc(COC(*)=O)cc1O[C@@H]1O[C@H](C(=O)NCC(=O)NCCCCCCOP(=O)(O)OC2CCC(c3ccccc3)(c3ccccc3)CC2)[C@@H](O)[C@H](O)[C@H]1O ; *Nc1ccc(COC(*)=O)cc1O[C@@H]1O[C@H](C(=O)N2CCC(C(=O)O)CC2)[C@@H](O)[C@H](O)[C@H]1O]

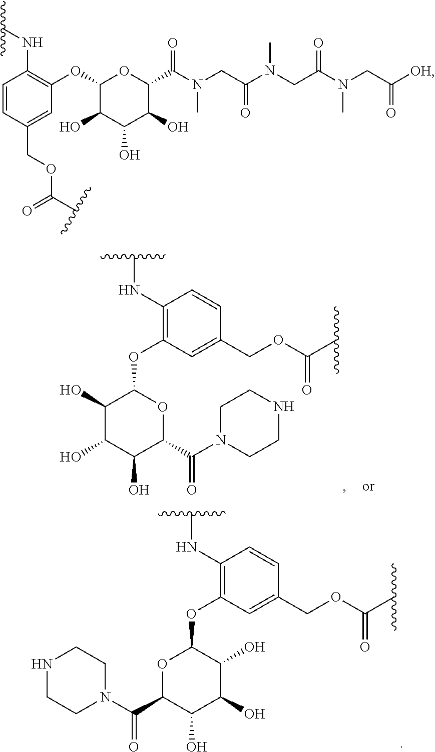

In some embodiments, each A is independently selected from natural or unnatural amino acids, wherein any free amine of an amino acid is optionally independently substituted with CH₃, wherein the natural or unnatural amino acids are selected from alanine (Ala), Ala(SO₃H), 3-(1-piperidinyl)alanine, cyclohexylalanine, arginine (Arg), asparagine (Asn), aspartate (Asp), cysteine (Cys), glutamine (Gln), glutamatic acid (Glu), glycine (Gly), leucine (Leu), lysine(Lys), methionine (Met), phenylalanine (Phe), homophenylalanine, proline (Pro), serine (Ser), 3-homoserine, tyrosine(Tyr), Tyr(SOH), tryptophan (Trp), valine (Val), citrulline, β-alanine, β3-homoserine, β3-homolysine, and β3-homoglutamic acid.

In one aspect, disclosed herein is a compound selected from Compound 57, 58, 64, 66, 79-82, 84, 85, 92-197, 239-245, 248, and 249, or a pharmaceutically acceptable salt thereof.

In one aspect, described herein is a pharmaceutical composition including an NDC disclosed herein, or a pharmaceutically acceptable salt thereof, and a pharmaceutically acceptable excipient.

In one aspect, described herein is a method for the treatment of cancer by administering to a mammal with cancer an effective amount of an NDC disclosed herein, or a pharmaceutically acceptable salt thereof.

Other objects, features and advantages of the compounds, methods and compositions described herein will become apparent from the following detailed description. It should be understood, however, that the detailed description and the specific examples, while indicating specific embodiments, are given by way of illustration only, since various changes and modifications within the spirit and scope of the instant disclosure will become apparent to those skilled in the art from this detailed description.

### DETAILED DESCRIPTION OF THE INVENTION

Cancer, a disease in which some cells undergo a genetic change in the control of their growth and replication that results in uncontrolled growth and spreading, is one of the leading causes of death worldwide. General types of cancers include solid tumors (cancers that typically originate in organs), carcinomas (cancers that originate in skin or tissues that line organs), sarcomas (cancers of connective tissues such as bones), leukemias (cancers of bone marrow), and lymphomas and myelomas (cancers of the immune system). Neoplasms are abnormal growth of cells that result in solid tumors which may be benign (i.e., do not display malignant features and are generally unlikely to become dangerous such as adenomas), malignant (i.e., display features such as genetic mutations, loss of normal function, rapid division, and can metastasize (invade) to other tissues), and of uncertain or unknown behavior. State-of-the-art treatment of neoplasms is accomplished by a combination of surgical procedures, chemotherapy, and radiation therapy. Surgical procedures can be curative under some conditions, but often requires multiple interventions as well as combination with radiation and chemotherapy. Chemotherapy proves to be a potent weapon in the fight against cancer in many cases, but further optimization is required. Chemotherapy is typically performed by systemic administration of potent cytotoxic drugs, but these compounds lack tumor selectivity and therefore also kill healthy cells in the body. This non-specific toxicity causes the severe side effects commonly associated with of chemotherapy. Radiotherapy is the use of high-energy radiation to kill cells. The source of radiation may be external-beam radiation (applied using an external source), internal radiation (placement of a radioactive material near the target cells), or radiotherapy from the systemic administration of a radioactive material. Similar to chemotherapy, many radiation therapy options also lack tumor cell identification properties needed to achieve the ultimate goal of targeted tumor therapy with drug molecules or radionuclides.

GPCRs are a large and diverse group of integral membrane receptors and as a consequence are expressed in every cell type in the body. GPCRs are generally poorly antigenic making them difficult targets for antibody-based strategies. For many GPCRs, a large proportion of the protein population resides in intracellular compartments at any given time, which reduces the total number of cell surface binding sites accessible to antibodies or peptides.

Many human tumors overexpress different GPCRs, often times at significantly higher density than other tissues. For example, gastroenteropancreatic (GEP) neuroendocrine tumors (NETs) overexpress somatostatin receptors, namely SSTR2, SSTR3, and SSTR5. Breast cancers also overexpress SSTR2. Due to the complicated GPCR overexpression profiles in neoplasms, targeting multiple receptors simultaneously may address issues such as heterogenity, resistance, and change of phenotype during disease progression that have hampered many current treatment options.

The class of somatostatin receptors (SSTRs) consists of five members (SSTR1, SSTR2, SSTR2, SSTR4, SSTR5), which are widely expressed in different tissues in the body including nervous, pituitary, kidney, lung, and immune cells. Their natural ligand is the neuropeptide somatostatin (SST), which occurs in two active isoforms, SST-14 and SST-28. In combination with their receptors, both isoforms act as inhibitory hormones. An important physiological function of the SSTR/SST axis is, for example, the inhibition of the release of growth hormones. SSTRs, particularly the SSTR subtype 2, are found highly expressed in many neoplastic cells and in tumoral blood vessels. Overexpression of SSTRs, in particular SSTR2, has been found in various neuroendocrine tumors, as well as other tumors such as breast, ovarian, and lung cancer. Targeting of the SSTR2 for drug delivery has been accomplished by using stabilized, cyclic somatostatin analogs such as octreotate, octreotide, and lanreotide. For example, covalently attaching a DOTA chelator to octreotide (DOTA-TATE, also known as DOTA-(Tyr³)-octreotate) has made it possible to target delivery of radionuclides to tumor cells expressing somatostatin receptors. ¹⁷⁷Lu DOTA-TATE therapy is a form of peptide receptor radionuclide therapy (PRRT) which targets somatostatin receptors and is a form of targeted drug delivery.

Most currently available GPCR-targeting drugs act at receptors for which the native ligands are small molecules, such as histamine, adrenaline, and neurotransmitters. Drugs targeting GPCR for which the native ligands are peptides or proteins, are typically also peptides or proteins.

Peptides are intrinsically sensitive to proteolytic enzymes and peptidases present in most tissues, and are rapidly degraded into multiple fragments which no longer have significant affinity for the intended receptors. There are ways to stabilize peptides (e.g., incorporating peptidomimetic structures or using more stable D amino acids in the peptide backbone) but these modifications can lead to loss of affinity and/or selectivity, and negatively impact physicochemical properties (e.g., poor solubility and tendency to aggregate). In addition, peptides may cause unwanted immunogenic responses complicating later stages of development by masking the therapeutic effect and impacting the safety assessment.

When peptide ligands are linked to cytotoxic payloads, the resulting conjugates often degrade rapidly in blood plasma and produce cytotoxic peptide fragments which may nonspecifically bind to both tumor and normal tissue. This premature breakdown of peptide drug conjugates (PDCs) and antibody drug conjugates (ADCs) reduces the amount of cytotoxic payloads distributed to targeted tumors, lowering treatment efficacy, and possibly increasing toxicity. In addition, peptides are most likely exclusively excreted via the kidneys, which may limit PDC applications. Marked kidney uptake of some peptide-based therapeutics has limited their routine use.

High affinity, small molecule ligands that bind peptide GPCRs and protein GPCRs, such as chemokine GPCRs, have been described and are cell permeable and can access populations of receptors in the endoplasmic reticulum and endosomes. Owing to the low molecular weight of non-peptide small molecules, vascular permeability and tumor penetration should be improved compared to high molecular weight conjugates based on peptides and antibodies. The binding affinity of small molecule nonpeptide ligands in many cases surpasses that of FDA approved antibodies by orders of magnitude.

Provided herein are NDCs which are SST2R ligand drug conjugates. The conjugated drug cargo or payload moiety is connected to the ligand in a manner that does not affect the binding affinity of the small molecule SST2R ligand to the SST2R. The conjugated drug cargos or payload moieties include chemotherapeutic agents, which are connected to the ligand using a spacer and/or linker moiety.

Solid Tumors: Benign and/or Malignant Neoplasms (Cancer)

In one aspect, compounds of Formula (I) or other NDCs disclosed herein are used to treat benign and/or malignant neoplasms (solid tumors), wherein the neoplasm comprises cells that overexpress cell surface GPCRs.

The term “neoplasm” as used herein, refers to an abnormal growth of cells that may proliferate in an uncontrolled way and may have the ability to metastasize (spread).

Neoplasms include solid tumors, adenomas, carcinomas, sarcomas, leukemias and lymphomas, at any stage of the disease with or without metastases.

A solid tumor is an abnormal mass of tissue that usually does not contain cysts or liquid areas. Solid tumors may be benign (not cancer), or malignant (cancer). Different types of solid tumors are named for the type of cells that form them. Examples of solid tumors are sarcomas, carcinomas, and lymphomas. Leukemias (cancers of the blood) generally do not form solid tumors.

Solid tumors are cancers that typically originate in organs, such as the bladder, bowel, brain, breast, endometrium, heart, kidney, lung, liver, uterus, ovaries, pancreas or other endocrine organs (thyroid), and prostate.

An adenoma is a tumor that is not cancer. It starts in gland-like cells of the epithelial tissue (thin layer of tissue that covers organs, glands, and other structures within the body). An adenoma can grow from many glandular organs, including the adrenal glands, pituitary gland, thyroid, prostate, and others. Over time adenomas may become malignant, at which point they are called adenocarcinomas. While benign, they have the potential to cause serious health complications by compressing other structures (mass effect) and by producing large amounts of hormones in an unregulated, non-feedback-dependent manner (causing paraneoplastic syndromes).

Adenomas typically are found in the colon (e.g. adenomatous polyps, which have a tendency to become malignant and to lead to colon cancer), kidneys (e.g. renal adenomas may be precursor lesions to renal carcinomas), adrenal glands (e.g. adrenal adenomas, such as pheochromocytoma; some secrete hormones such as cortisol, causing Cushing's syndrome, aldosterone causing Conn's syndrome, or androgens causing hyperandrogenism), thyroid (e.g. thyroid adenoma), pituitary (e.g. pituitary adenomas, such as prolactinoma), parathyroid (e.g. an adenoma of a parathyroid gland may secrete inappropriately high amounts of parathyroid hormone and thereby cause primary hyperparathyroidism), liver (e.g. hepatocellular adenoma), breast (e.g. fibroadenomas), appendix (e.g. cystadenoma), bronchial (e.g. bronchial adenomas may cause carcinoid syndrome, a type of paraneoplastic syndrome), prostate (e.g. prostate adenoma), sebaceous gland (e.g. sebaceous adenoma), and salivary glands.

Metastasis is the spread of malignant cells to new areas of the body, often by way of the lymph system or bloodstream. A metastatic tumor is one that has spread from the primary site of origin, or where it started, into different areas of the body. Metastatic tumors comprise malignant cells that express cell surface GPCRs.

Tumors formed from cells that have spread are called secondary tumors. Tumors may have spread to areas near the primary site, called regional metastasis, or to parts of the body that are farther away, called distant metastasis.

In some embodiments, the tumor to be treated comprises tumor cells expressing a GPCR, wherein the tumor is a primary or metastatic tumor. In some embodiments, the tumor to be treated comprises tumor cells expressing a GPCR, wherein the tumor is a primary or metastatic tumor of gastrointestinal origin, such as colorectal cancer, stomach cancer, small intestine cancer, or esophageal cancer. In some embodiments, the tumor to be treated comprises tumor cells expressing a GPCR, wherein the tumor is a primary or metastatic tumor of the pancreas. In some embodiments, the tumor to be treated comprises tumor cells expressing a GPCR, wherein the tumor is a primary or metastatic tumor of the lungs, such as squamous cell carcinoma, adenosquamous carcinoma, or adenocarcinoma. In some embodiments, the tumor to be treated comprises tumor cells expressing a GPCR, wherein the tumor is a primary or metastatic neuroectodermal tumor, such as aphaechromotcytoma or a paraganglioma. In some embodiments, the tumor to be treated comprises tumor cells expressing a GPCR, wherein the tumor is a primary or metastatic bronchopulmonary or gastrointestinal neuroendocrine tumor. In some embodiments, the tumor to be treated comprises tumor cells expressing a GPCR, wherein the tumor is a primary or metastatic tumor of the rectum or colon.

In some embodiments, compounds of Formula (I) or other NDC disclosed herein are used to treat a sarcoma, such as leiomyosarcoma or rhabdomyosarcoma.

In some embodiments, compounds of Formula (I) or other NDC disclosed herein are used to treat an adenoma.

In another aspect, provided herein is a method for treating cancer in a mammal comprising administering to the mammal in need thereof compounds of Formula (I) or other NDC disclosed herein. In some embodiments, the cancer comprises tumor cells expressing one or more peptide hormone GPCRs. In some embodiments, the cancer comprises tumor cells that overexpress one or more GPCRs. In some embodiments, the cancer comprises a solid tumor. In some embodiments, the cancer comprises a sarcoma, carcinoma, or lymphoma. In some embodiments, the cancer comprises a neuroendocrine tumor. In some embodiments, the cancer comprises an insulinoma. In some embodiments, the cancer comprises peptide hormone GPCR-positive (e.g., somatostatin receptor-positive) gastroenteropancreatic neuroendocrine tumors (GEP-NETs).

In some embodiments, the compounds of Formula (I) or other NDC disclosed herein are administered to an oncology patient. In some embodiments, the oncology patient has been diagnosed with a carcinoma, sarcoma, primary tumor, metastatic tumor, solid tumor, non-solid tumor, blood tumor, leukemia or lymphoma.

Carcinomas include, but are not limited to, esophageal carcinoma, hepatocellular carcinoma, basal cell carcinoma (a form of skin cancer), squamous cell carcinoma (various tissues), bladder carcinoma, including transitional cell carcinoma (a malignant neoplasm of the bladder), bronchogenic carcinoma, colon carcinoma, colorectal carcinoma, gastric carcinoma, lung carcinoma, including small cell carcinoma and non-small cell carcinoma of the lung, adrenocortical carcinoma, thyroid carcinoma, pancreatic carcinoma, breast carcinoma, ovarian carcinoma, prostate carcinoma, adenocarcinoma, sweat gland carcinoma, sebaceous gland carcinoma, papillary carcinoma, papillary adenocarcinoma, cystadenocarcinoma, medullary carcinoma, renal cell carcinoma, ductal carcinoma in situ or bile duct carcinoma, choriocarcinoma, seminoma, embryonal carcinoma, Wilm's tumor, cervical carcinoma, uterine carcinoma, testicular carcinoma, osteogenic carcinoma, epithelial carcinoma, and nasopharyngeal carcinoma, etc.

Sarcomas include, but are not limited to, fibrosarcoma, myxosarcoma, liposarcoma, chondrosarcoma, chordoma, osteogenic sarcoma, osteosarcoma, angiosarcoma, endotheliosarcoma, lymphangiosarcoma, lymphangioendotheliosarcoma, synovioma, mesothelioma, Ewing's sarcoma, leiomyosarcoma, rhabdomyosarcoma, and other soft tissue sarcomas.

Solid tumors include, but are not limited to, glioma, astrocytoma, medulloblastoma, craniopharyngioma, ependymoma, pinealoma, hemangioblastoma, acoustic neuroma, oligodendroglioma, menangioma, melanoma, neuroblastoma, and retinoblastoma. Benign solid tumors include adenomas.

Leukemias include, but are not limited to, a) chronic myeloproliferative syndromes (neoplastic disorders of multipotential hematopoietic stem cells); b) acute myelogenous leukemias (neoplastic transformation of a multipotential hematopoietic stem cell or a hematopoietic cell of restricted lineage potential; c) chronic lymphocytic leukemias (CLL; clonal proliferation of immunologically immature and functionally incompetent small lymphocytes), including B-cell CLL. T-cell CLL prolymphocyte leukemia, and hairy cell leukemia; and d) acute lymphoblastic leukemias (characterized by accumulation of lymphoblasts). Lymphomas include, but are not limited to, B-cell lymphomas (e.g., Burkitt's lymphoma); Hodgkin's lymphoma; and the like.

Primary and metastatic tumors include, e.g., lung cancer (including, but not limited to, lung adenocarcinoma, squamous cell carcinoma, large cell carcinoma, bronchioloalveolar carcinoma, non-small-cell carcinoma, small cell carcinoma, mesothelioma); breast cancer (including, but not limited to, ductal carcinoma, lobular carcinoma, inflammatory breast cancer, clear cell carcinoma, mucinous carcinoma); colorectal cancer (including, but not limited to, colon cancer, rectal cancer); anal cancer; pancreatic cancer (including, but not limited to, pancreatic adenocarcinoma, islet cell carcinoma, neuroendocrine tumors); prostate cancer; ovarian carcinoma (including, but not limited to, ovarian epithelial carcinoma or surface epithelial-stromal tumor including serous tumor, endometrioid tumor and mucinous cystadenocarcinoma, sex-cord-stromal tumor); liver and bile duct carcinoma (including, but not limited to, hepatocellular carcinoma, cholangiocarcinoma, hemangioma); esophageal carcinoma (including, but not limited to, esophageal adenocarcinoma and squamous cell carcinoma); non-Hodgkin's lymphoma; bladder carcinoma; carcinoma of the uterus (including, but not limited to, endometrial adenocarcinoma, uterine papillary serous carcinoma, uterine clear-cell carcinoma, uterine sarcomas and leiomyosarcomas, mixed mullerian tumors); glioma, glioblastoma, medulloblastoma, and other tumors of the brain; kidney cancers (including, but not limited to, renal cell carcinoma, clear cell carcinoma, Wilm's tumor); cancer of the head and neck (including, but not limited to, squamous cell carcinomas); cancer of the stomach (including, but not limited to, stomach adenocarcinoma, gastrointestinal stromal tumor); thymus cancer, multiple myeloma; testicular cancer; germ cell tumor; neuroendocrine tumor; cervical cancer; carcinoids of the gastrointestinal tract, breast, and other organs; and signet ring cell carcinoma.

### Non-Peptide Drug Conjugates

In one aspect, described herein is a non-peptide drug conjugate, or NDC.

In some embodiments, the NDC is a compound having the structure of Formula (I), or a pharmaceutically acceptable salt thereof:

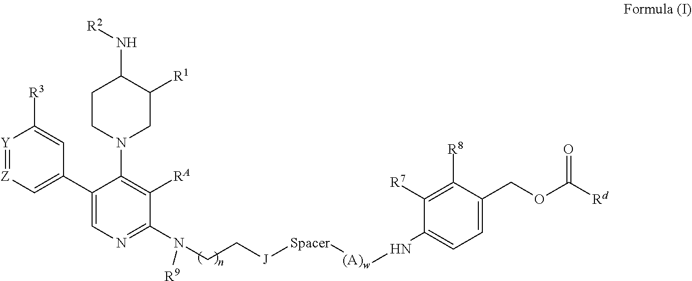

wherein:
J is a bond or NR⁶;
n is an integer selected from 1-5;
R^{A} is

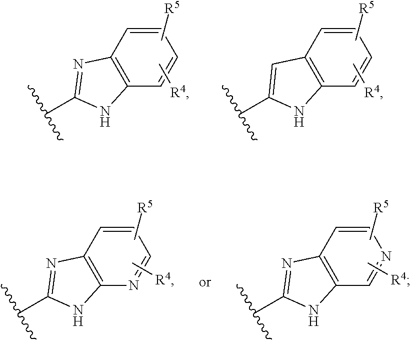

Y is N or CH;
Z is N or CR¹⁰;
R¹ is H;
R² is H or C₁-C₆ alkyl; or
R¹ and R², together with the N and C to which they are attached, combine to form a heterocycloalkyl;
R³ is C₁-C₆ alkyl or C₁-C₆ alkoxy;
R⁴ is H, halogen, CN, or C₁-C₆ alkoxy;
R⁵ is H, halogen, or CN;
R⁶ is H or C₁-C₆ alkyl;
R⁹ is H or C₁-C₆ alkyl;
R¹⁰ is H or halogen;
Spacer is a spacer group;
each A is independently selected from natural or unnatural amino acids, wherein any free amine of an amino acid is optionally independently substituted with CH₃;
w is an integer selected from 1-6;
R⁷ is H, C₁-C₆ haloalkyl, OR^{7a},

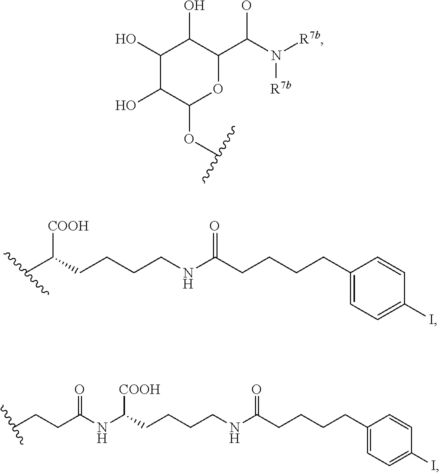

or C₁-C₆ alkyl optionally substituted with COOH;
R^{7a} is H, or C₁-C₆ alkyl optionally substituted with amino, COOH, or OH;
R^{7b} is H, C₁-C₂₀ alkyl optionally substituted with OR^{7b1}, COOR^{7b1}, NR^{7b1}₂, or SO₃R^{7b1}; (CH₂)₂NHC(═O)—R^{7b2},

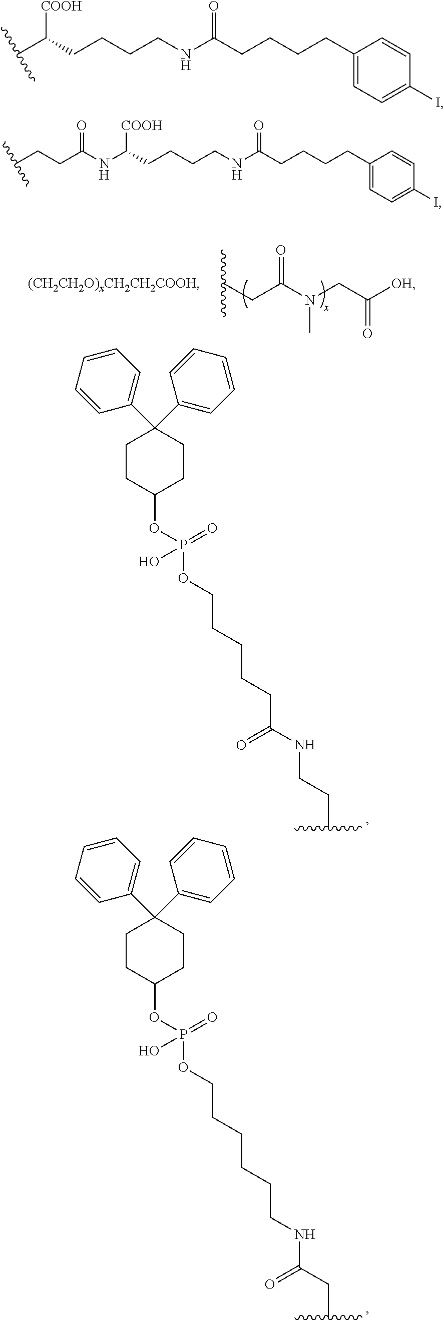

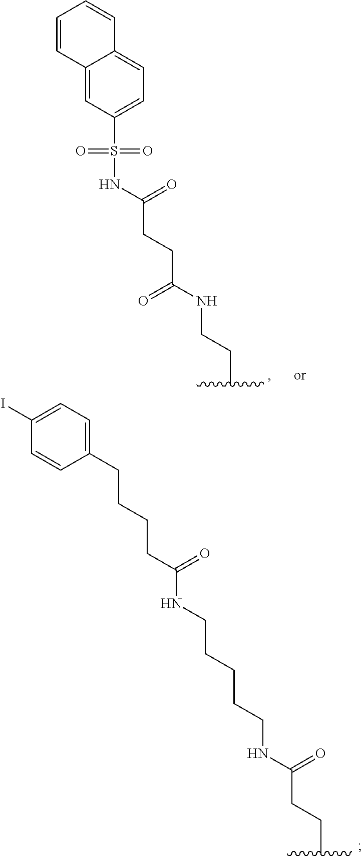 [SMILES: *CCNC(=O)CCC(=O)NS(=O)(=O)c1ccc2ccccc2c1 ; *CCC(=O)NCCCCCNC(=O)CCCCc1ccc(I)cc1]

each R^{7b1} is independently selected from H and C₁-C₆ alkyl;
R^{7b2} is (CH₂CH₂O)ₓCH₂CH₂COOH, or (CH₂)_{y}COOH;
R^{7c} is H or C₁-C₆ alkyl; or
R^{7b} and R^{7c}, together with the nitrogen to which they are attached, combine to form a heterocycloalkyl group optionally substituted with amino, COOH, or C₁-C₆ alkyl which is optionally substituted with OH or COOH;
R⁸ is H, C₁-C₆ haloalkyl, COOH, C(═O)NH—R^{8a}

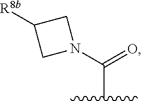

or C(═O)NH(CH₂)₂NHC(═O)—R^{8c};
R^{8a} is C₁-C₂₀ alkyl substituted with COOH or SO₃H, or C₁-C₆ alkoxy optionally substituted with amino;
R^{8b} is COOH or amino;
R^{8c} is (CH₂CH₂O)ₓCH₂CH₂COOH or (CH₂)_{y}COOH;
each x is independently an integer selected from 1-10;
each y is independently an integer selected from 1-20;
R^{d} is the residue of a bioactive molecule of the formula R^{d}—H;
wherein when R⁷ is H, then R⁸ is C₁-C₆ haloalkyl, COOH, C(═O)NH—R^{8a},

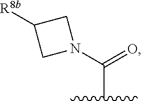

or C(═O)NH(CH₂)₂NHC(═O)—R^{8c}; and
wherein when R⁸ is H, then R⁷ is C₁-C₆ haloalkyl, OR^{7a},

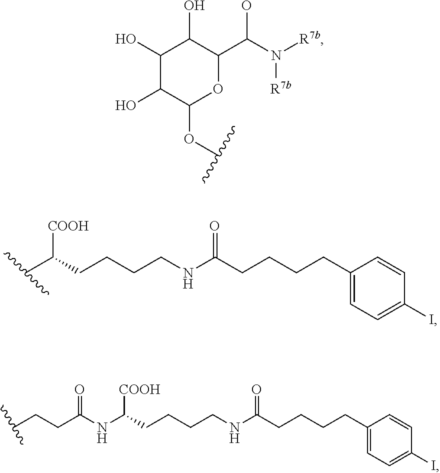

or C₁-C₆ alkyl optionally substituted with COOH.

In some embodiments, R⁷ is H, C₁-C₃ haloalkyl, OR^{7a},

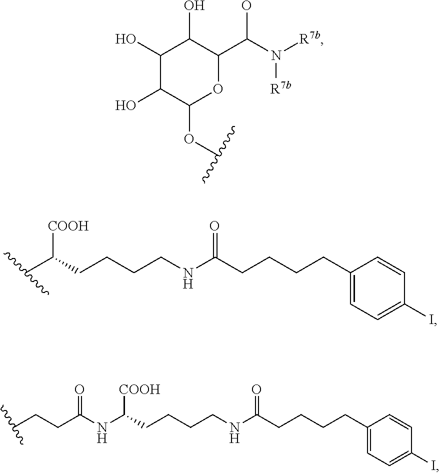

or C₁-C₃ alkyl optionally substituted with COOH;
R^{7a} is H, or C₁-C₃ alkyl optionally substituted with amino, COOH, or OH;
H, C₁-C₁₄ alkyl optionally substituted with OR^{7b1}, COOR^{7b1}, NR^{7b1}₂, or SO₃R^{7b1}; (CH₂)₂NHC(═O)—R^{7b2},

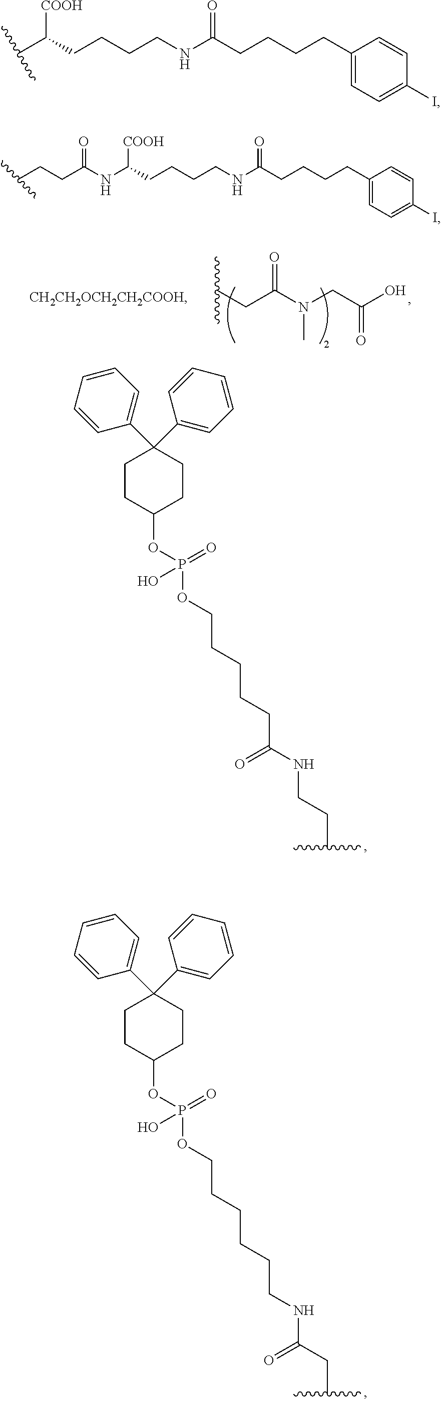

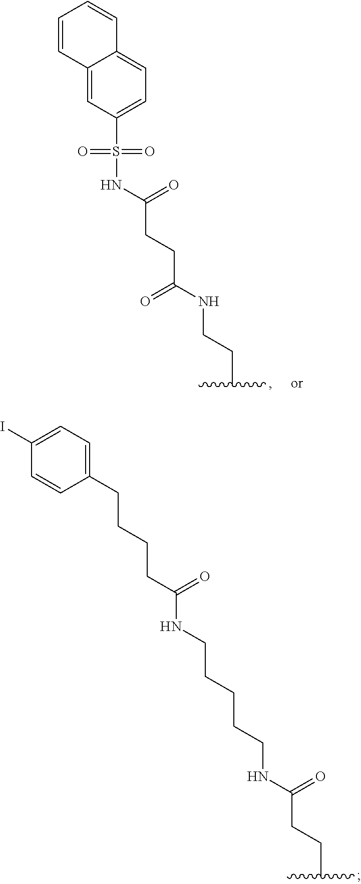 [SMILES: *CCNC(=O)CCC(=O)NS(=O)(=O)c1ccc2ccccc2c1 ; *CCC(=O)NCCCCCNC(=O)CCCCc1ccc(I)cc1]

R^{7b1} is H or C₁-C₃ alkyl;
R^{7b2} is (CH₂CH₂O)ₓCH₂CH₂COOH or (CH₂)_{y}COOH;
R^{7c} is H or C₁-C₃ alkyl; or
R^{7b} and R^{7c}, together with the nitrogen to which they are attached, combine to form a 3-6-membered heterocycloalkyl group optionally substituted with amino, COOH, or C₁-C₃ alkyl which is optionally substituted with OH or COOH;
R⁸ is H, C₁-C₃ haloalkyl, COOH, C(═O)NH—R^{8a},

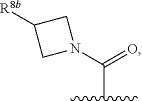

or C(═O)NH(CH₂)₂NHC(═O)—R^{8c};
R^{8a} is C₁-C₁₀ alkyl substituted with COOH or SO₃H, or C₁-C₃ alkoxy optionally substituted with amino;
R^{8b} is COOH or amino;
R^{8c} is (CH₂CH₂O)ₓCH₂CH₂COOH or (CH₂)_{y}COOH;
wherein when R⁷ is H, then R⁸ is C₁-C₃ haloalkyl, COOH, C(═O)NH—R^{8a},

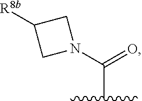

or C(═O)NH(CH₂)₂NHC(═O)—R^{8c}; and
wherein when R⁸ is H, then R⁷ is C₁-C₃ haloalkyl, OR^{7a},

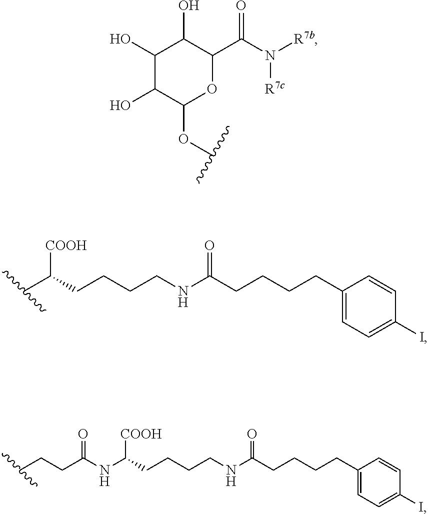

or C₁-C₃ alkyl optionally substituted with COOH

In some embodiments, the NDC is a compound having the structure of Formula (I), or a pharmaceutically acceptable salt thereof:
wherein:
J is a bond or NR⁶;
n is an integer selected from 1-5;
R^{A} is

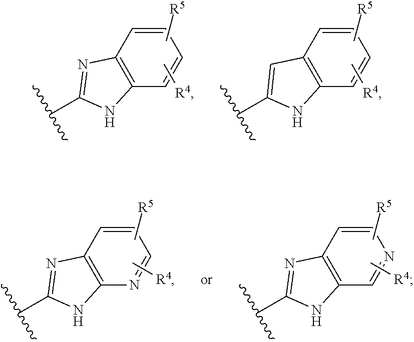

Y is N or CH;
Z is N or CR¹⁰;
R¹ is H;
R² is H or C₁-C₆ alkyl; or
R¹ and R², together with the N and C to which they are attached, combine to form a heterocycloalkyl;
R³ is C₁-C₆ alkyl or C₁-C₆ alkoxy;
R⁴ is H, halogen, CN, or C₁-C₆ alkoxy;
R⁵ is H, halogen, or CN;
R⁶ is H or C₁-C₆ alkyl;
R⁹ is H or C₁-C₆ alkyl;
R¹⁰ is H or halogen;
Spacer is a spacer group;
each A is independently selected from natural or unnatural amino acids, wherein any free amine of an amino acid is optionally independently substituted with CH₃;
w is an integer selected from 1-6;
R⁷ is H, C₁-C₆ alkyl, C₁-C₆ haloalkyl, OR^{7′}, or

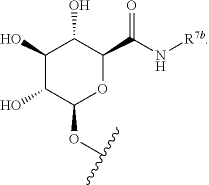

R^{7a} is H, or C₁-C₆ alkyl optionally substituted with amino, COOH, or OH; R^{7b} is H, C₁-C₂) alkyl substituted with COOR^{7b1}, amino, or SO₃H; (CH₂)₂NHC(═O)—R^{7b2};

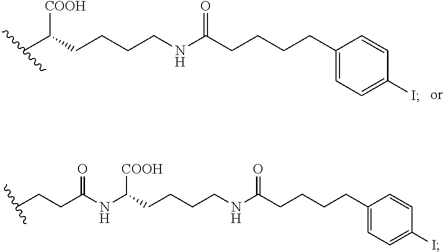 [SMILES: *[C@@H](CCCCNC(=O)CCCCc1ccc(I)cc1)C(=O)O ; "or" ; *CCC(=O)N[C@@H](CCCCNC(=O)CCCCc1ccc(I)cc1)C(=O)O]

R^{7b1} is H or C₁-C₆ alkyl;
R^{7b2} is (CH₂CH₂O)ₓCH₂CH₂COOH or (CH₂)_{y}COOH;
R⁸ is H, C₁-C₆ haloalkyl, COOH, C(═O)NH—R^{8a}

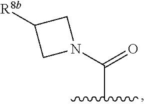

or C(═O)NH(CH₂)₂NHC(═O)—R^{8c};
R^{8a} is C₁-C₂₀ alkyl substituted with COOH or SO₃H; or C₁-C₆ alkoxy optionally substituted with amino;
R^{8b} is COOH or amino;
R^{8c} is (CH₂CH₂O)ₓCH₂CH₂COOH or (CH₂)_{y}COOH;
each x is independently an integer selected from 1-10;
each y is independently an integer selected from 1-20;
R^{d} is the residue of a bioactive molecule of the formula R^{d}—H;
wherein when R⁷ is H, then R⁸ is C₁-C₆ haloalkyl, COOH, C(═O)NH—R^{8a},

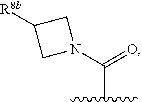

or C(═O)NH(CH₂)₂NHC(═O)—R^{8c}; and
wherein when R⁸ is H, then R⁷ is C₁-C₆ alkyl, C₁-C₆ haloalkyl, OR^{7a}, or

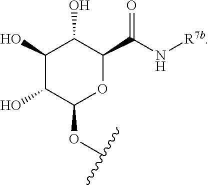

In some embodiments, Y is N. In some embodiments, Y is CH.

In some embodiments, Z is N. In some embodiments, Z is CR¹⁰.

In some embodiments, when Y is N, then Z is CR¹⁰. In some embodiments, when Y is CH, then Z is N. In some embodiments, when Y is CH, then Z is CR¹⁰.

In some embodiments, R¹⁰ is H. In some embodiments, R¹⁰ is halogen. In some embodiments, R¹⁰ is H or F. In some embodiments. R¹⁰ is F.

In some embodiments, R^{A} is

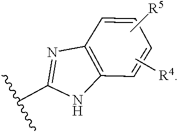

In some embodiments, R^{A} is

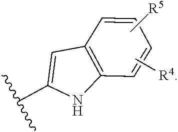

In some embodiments, R^{A} is

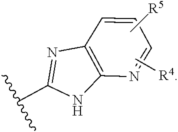

In some embodiments. R^{A} is

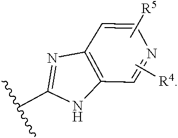

In some embodiments, R¹ is hydrogen.

In some embodiments, R² is H. In some embodiments, R² is C₁-C₆ alkyl. In some embodiments, R² is H or C₁-C₃ alkyl. In some embodiments, R² is C₁-C₃ alkyl. In some embodiments, R² is methyl, ethyl, propyl, or isopropyl. In some embodiments. R² is H or methyl. In some embodiments, R² is methyl.

In some embodiments, R¹ and R², together with the N and C to which they are attached, combine to form a heterocycloalkyl. In some embodiments, R¹ and R², together with the N and C to which they are attached, combine to form a 6-membered heterocycloalkyl. In some embodiments, R¹ and R², together with the N and C to which they are attached, combine to form a morpholine or a piperidine. In some embodiments, R¹ and R², together with the N and C to which they are attached, combine to form a morpholine. In some embodiments, R¹ and R², together with the N and C to which they are attached, combine to form a piperidine.

In some embodiments, R³ is C₁-C₆ alkyl or C₁-C₆ alkoxy. In some embodiments, R³ is C₁-C₆ alkyl. In some embodiments, R³ is C₁-C₆ alkoxy. In some embodiments, R³ is C₁-C₃ alkyl or C₁-C₃ alkoxy. In some embodiments, R³ is C₁-C₃ alkyl. In some embodiments. R³ is C₁-C₃ alkoxy. In some embodiments, is R³ is methyl, ethyl, propyl, isopropyl, methoxy, ethoxy, propoxy, or isopropoxy. In some embodiments, R³ is methyl, ethyl, propyl, or isopropyl. In some embodiments, is R³ is methoxy, ethoxy, propoxy, or isopropoxy. In some embodiments, R³ is methyl or methoxy. In some embodiments, is R³ is methoxy. In some embodiments, R³ is methyl.

In some embodiments, R⁴ is H, halogen, CN, or C₁-C₃ alkoxy. In some embodiments, R⁴ is H. In some embodiments, R⁴ is CN. In some embodiments, R⁴ is C₁-C₃ alkoxy. In some embodiments, R⁴ is methoxy, ethoxy, propoxy, or isopropoxy. In some embodiments, R⁴ is methoxy. In some embodiments, R⁴ is halogen. In some embodiments, R⁴ is F or Cl. In some embodiments, R⁴ is F. In some embodiments, R⁴ is C₁. In some embodiments, R⁴ is H, F, Cl, OMe, or CN.

In some embodiments, R⁵ is H, halogen, or CN. In some embodiments, R⁵ is H. In some embodiments, R⁵ is CN. In some embodiments, R⁵ is halogen. In some embodiments, R⁵ is F or Cl. In some embodiments, R⁵ is F. In some embodiments, R⁵ is Cl. In some embodiments, R⁵ is H, F, Cl, or CN.

In some embodiments, R^{A} of Formula (I) is

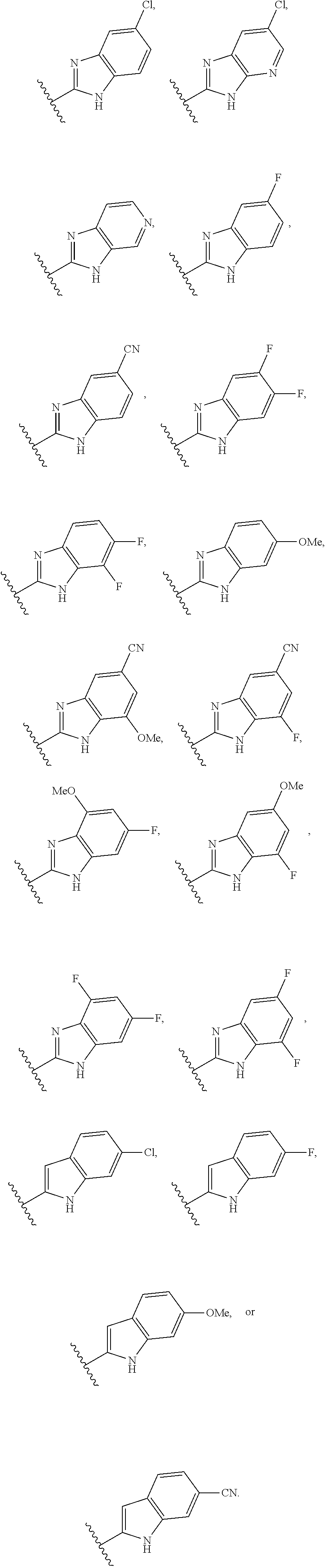

In some embodiments, R^{A} of Formula (I) is

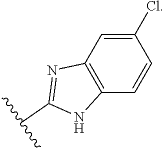 [SMILES: *c1nc2cc(Cl)ccc2[nH]1]

In some embodiments,

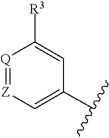

### of Formula (I) is

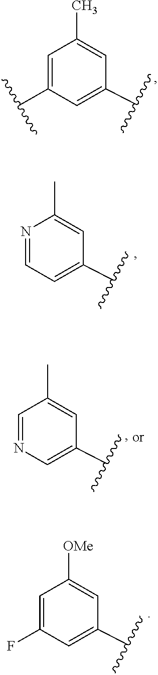

In some embodiments,

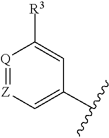

### of Formula (I) is

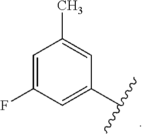 [SMILES: *c1cc(C)cc(F)c1]

In some embodiments, J is a bond. In some embodiments, J is NR⁶.

In some embodiments, R⁶ is H. In some embodiments, R⁶ is C₁-C₆ alkyl. In some embodiments, R⁶ is H or C₁-C₃ alkyl. In some embodiments, R⁶ is H, methyl, ethyl, propyl, or isopropyl. In some embodiments, R⁶ is H or methyl. In some embodiments, R⁶ is methyl.

In some embodiments, J is NR⁶ and R⁶ is H or methyl. In some embodiments, J is NR⁶ and R⁶ is H. In some embodiments, J is NR⁶ and R⁶ is methyl.

In some embodiments, R⁹ is H. In some embodiments, R⁹ is C₁-C₆ alkyl. In some embodiments, R⁹ is H or C₁-C₃ alkyl. In some embodiments, R⁹ is H, methyl, ethyl, propyl, or isopropyl. In some embodiments, R⁹ is H or methyl. In some embodiments, R⁹ is methyl.

In some embodiments, R⁹ is H or methyl, and J is NH or NMe. In some embodiments, R⁹ is H and J is NH.

In some embodiments, n is 1. In some embodiments, n is 2. In some embodiments, n is 3. In some embodiments, n is 4. In some embodiments, n is 5.

In some embodiments, J is NR⁶ and n is 1.

In some embodiments, the compound of Formula (I) is a compound of Formula (Ia):

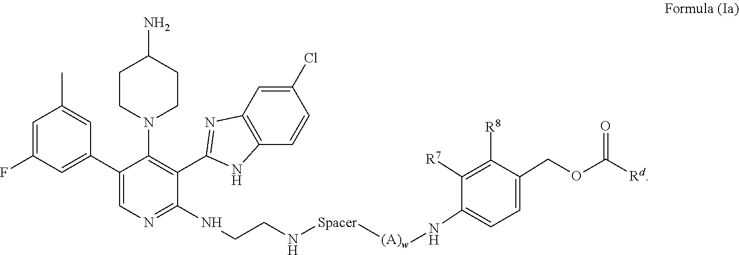

In some embodiments, the compound of Formula (I) is a compound of Formula (Ib):

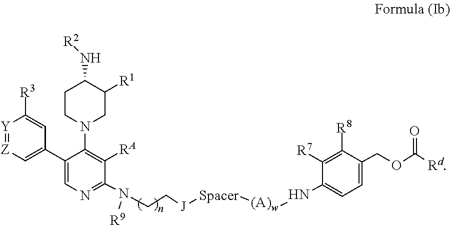

In some embodiments:
R⁷ is H, C₁-C₃ alkyl, C₁-C₃ haloalkyl, OR^{7a}, or

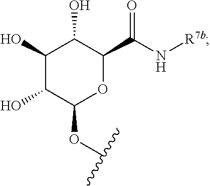

R^{7a} is H, or C₁-C₃ alkyl optionally substituted with amino, COOH, or OH,
R^{7b} is H; C₁-C₁₄ alkyl substituted with COOR^{7b1}, amino, or SO₃H; (CH₂)₂NHC(═O)—R^{7b2};

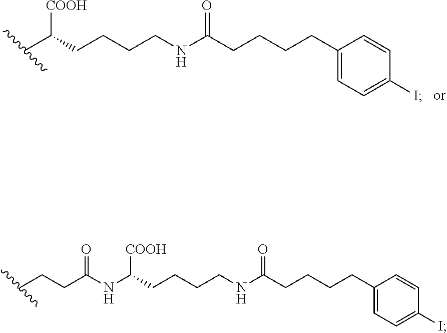

R^{7b1} is H or C₁-C₃ alkyl;
R^{7b2} is (CH₂CH₂O)ₓCH₂CH₂COOH, or (CH₂)_{y}COOH;
R⁸ is H, C₁-C₃ haloalkyl, COOH, C(═O)NH—R^{8a},

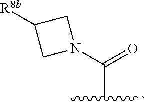

or C(═O)NH(CH₂)₂NHC(═O)—R^{8c};
R^{8a} is C₁-C₁₀ alkyl substituted with COOH or SO₃H; or C₁-C₃ alkoxy optionally substituted with amino;
R^{8b} is COOH or amino;
R^{8c} is (CH₂CH₂O)ₓCH₂CH₂COOH or (CH₂)_{y}COOH;
wherein when R⁷ is H, then R⁸ is C₁-C₃ haloalkyl, COOH, C(═O)NH—R^{8a},

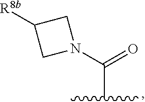

or C(═O)NH(CH₂)₂NHC(═O)—R^{8c}; and
wherein when R⁸ is H, then R⁷ is C₁-C₃ alkyl, C₁-C₃ haloalkyl, OR^{7a}, or

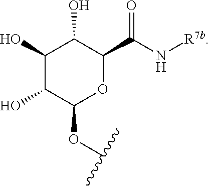

In some embodiments, R⁷ is C₁-C₃ alkyl. In some embodiments. R⁷ is methyl, ethyl, propyl, or isopropyl. In some embodiments, R⁷ is methyl.

In some embodiments. R⁷ is C₁-C₃ haloalkyl. In some embodiments, R⁷ is trifluoromethyl.

In some embodiments, R⁷ is OR^{7a}. In some embodiments, R^{7a} is H. In some embodiments, R^{7a} is C₁-C₃ alkyl. In some embodiments, R^{7a} is methyl, ethyl, propyl, or isopropyl. In some embodiments. R^{7a} is C₁-C₃ alkyl substituted with amino. In some embodiments, R^{7a} is methyl, ethyl, propyl, or isopropyl, any of which is substituted with amino. In some embodiments, R^{7a} is ethyl substituted with amino. In some embodiments, R^{7a} is C₁-C₃ alkyl substituted with COOH. In some embodiments, R^{7a} is methyl, ethyl, propyl, or isopropyl, any of which is substituted with COOH. In some embodiments. R^{7a} is methyl substituted with COOH. In some embodiments, R^{7a} is C₁-C₃ alkyl substituted with OH. In some embodiments, R^{7a} is methyl, ethyl, propyl, or isopropyl, any of which is substituted with OH. In some embodiments, R^{7a} is ethyl substituted with OH. In some embodiments, R^{7a} is propyl substituted with OH.

In some embodiments, R⁷ is

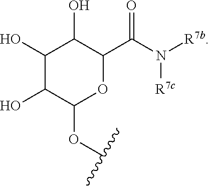

In some embodiments, R^{7c} is H. In some embodiments, R^{7c} is C₁-C₆ alkyl. In some embodiments, R^{7c} is C₁-C₃ alkyl. In some embodiments, R^{7c} is H, methyl, ethyl, or propyl. In some embodiments, R^{7c} is methyl.

In some embodiments, R⁷ is

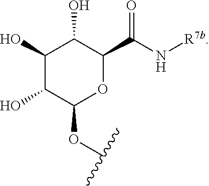

In some embodiments, R⁷ is C₁-C₆ alkyl optionally substituted with COOH. In some embodiments. R⁷ is C₁-C₃ alkyl optionally substituted with COOH. In some embodiments, R⁷ is methyl, ethyl, or propyl optionally substituted with COOH. In some embodiments, R⁷ is ethyl substituted with COOH.

In some embodiments, R⁷ is

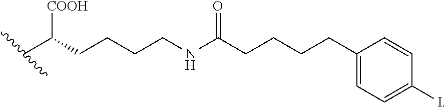 [SMILES: *[C@@H](CCCCNC(=O)CCCCc1ccc(I)cc1)C(=O)O]

In some embodiments, R⁷ is

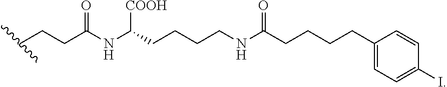

In some embodiments. R^{7b} is H.

In some embodiments, R^{7b} is C₁-C₂₀ alkyl optionally substituted with OR^{7b1}, COOR^{7b1}, NR^{7b1}₂, or SO₃R^{7b1}. In some embodiments, R^{7b} is C₁-C₁₄ alkyl optionally substituted with OR^{7b1}, COOR^{7b1}, NR^{7b1}₂, or SO₃R^{7b1}. In some embodiments, R^{7b} is C₁-C₆ alkyl optionally substituted with OR^{7b1}, COOR^{7b1}, NR^{7b1}₂, or SO₃R^{7b1}. In some embodiments, R^{7b} is C₁-C₃ alkyl optionally substituted with OR^{7b1}, COOR^{7b1}, NR^{7b1}₂, or SO₃R^{7b1}. In some embodiments, R^{7b} is ethyl optionally substituted with OR^{7b1}, COOR^{7b1}, NR^{7b1}₂, or SO₃R^{7b1}. In some embodiments, R^{7b} is ethyl optionally substituted with OR^{7b1}. In some embodiments, R^{7b} is ethyl optionally substituted with COOR^{7b1}. In some embodiments. R^{7b} is ethyl optionally substituted with NR^{7b1}₂. In some embodiments, R^{7b} is ethyl optionally substituted with SO₃R^{7b1}.

In some embodiments, R^{7b} is C₁-C₁₄ alkyl substituted with COOR^{7b1}, amino, or SO₃H. In some embodiments, R^{7b} is C₁-C₁₄ alkyl substituted with COOR^{7b1}. In some embodiments, R^{7b} is methyl substituted with COOR^{7b1}. In some embodiments, R^{7b} is ethyl substituted with COOR^{7b1}. In some embodiments, R^{7b} is C₁₄ substituted with COOR^{7b1}. In some embodiments, R^{7b} is C₁-C₁₄ alkyl substituted with amino. In some embodiments, R^{7b} is ethyl substituted with amino. In some embodiments, R^{7b} is C₁-C₁₄ alkyl substituted with SO₃H. In some embodiments, R^{7b} is methyl substituted with SO₃H. In some embodiments. R^{7b} is ethyl substituted with SO₃H.

In some embodiments, R^{7b1} is H or C₁-C₃ alkyl. In some embodiments, R^{7b1} is H. In some embodiments, R^{7b1} is C₁-C₃ alkyl. In some embodiments, R^{7b1} is methyl, ethyl, propyl, or isopropyl. In some embodiments, R^{7b1} is H or methyl. In some embodiments, R^{7b1} is methyl.

In some embodiments, R^{7b} is (CH₂)₂NHC(═O)—R^{7b2}. In some embodiments, R^{7b} is (CH₂CH₂O)ₓCH₂CH₂COOH. In some embodiments, R^{7b} is (CH₂)_{y}COOH.

In some embodiments, x is 1-10. In some embodiments, x is 2. In some embodiments, x is 1. In some embodiments, x is 3. In some embodiments, x is 4. In some embodiments, x is 5. In some embodiments, x is 6. In some embodiments, x is 7. In some embodiments, x is 8. In some embodiments, x is 9. In some embodiments, x is 10.

In some embodiments, y is 1-20. In some embodiments, y is 1. In some embodiments, y is 2. In some embodiments, y is 3. In some embodiments, y is 4. In some embodiments, y is 5. In some embodiments, y is 6. In some embodiments, y is 7. In some embodiments, y is 8. In some embodiments, y is 9. In some embodiments, y is 10. In some embodiments, y is 11. In some embodiments, y is 12. In some embodiments, y is 13. In some embodiments, y is 14. In some embodiments, y is 15. In some embodiments, y is 16. In some embodiments, y is 17. In some embodiments, y is 18. In some embodiments, y is 19. In some embodiments, y is 20.

In some embodiments. R^{7b} is

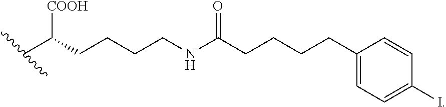 [SMILES: *[C@@H](CCCCNC(=O)CCCCc1ccc(I)cc1)C(=O)O]

In some embodiments. R^{7b} is

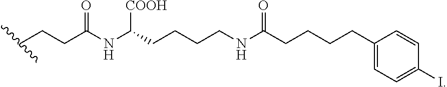

In some embodiments, R^{7b} is CH₂CH₂OCH₂CH₂COOH.

In some embodiments, R^{7b} is

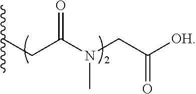

In some embodiments. R^{7b} is

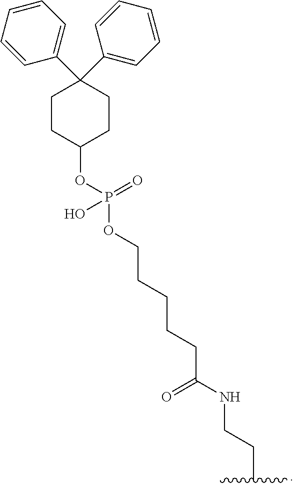 [SMILES: *CCNC(=O)CCCCCOP(=O)(O)OC1CCC(c2ccccc2)(c2ccccc2)CC1]

In some embodiments. R^{7b} is

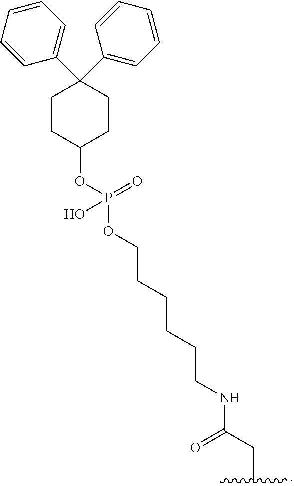 [SMILES: *CC(=O)NCCCCCCOP(=O)(O)OC1CCC(c2ccccc2)(c2ccccc2)CC1]

In some embodiments, R^{7b} is

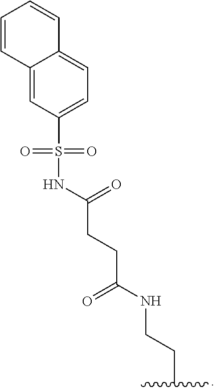 [SMILES: *CCNC(=O)CCC(=O)NS(=O)(=O)c1ccc2ccccc2c1]

In some embodiments, R^{7b} is

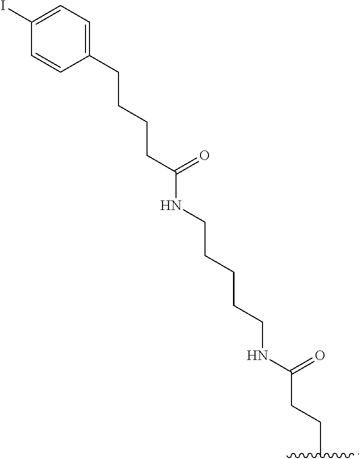 [SMILES: *CCC(=O)NCCCCCNC(=O)CCCCc1ccc(I)cc1]

In some embodiments, R^{7b} and R^{7c}, together with the nitrogen to which they are attached, combine to form a heterocycloalkyl group optionally substituted with amino, COOH or C₁-C₃ alkyl which is optionally substituted with OH or COOH. In some embodiments, R^{7b} and R^{7c}, together with the nitrogen to which they are attached, combine to form a 3-6-membered heterocycloalkyl group optionally substituted with amino. COOH or C₁-C₃ alkyl which is optionally substituted with OH or COOH. In some embodiments, R^{7b} and R^{7c}, together with the nitrogen to which they are attached, combine to form a 6-membered heterocycloalkyl group optionally substituted with amino, COOH or C₁-C₃ alkyl which is optionally substituted with OH or COOH.

In some embodiments, R^{7b} and R^{7c}, together with the nitrogen to which they are attached, combine to form a heterocycloalkyl group. In some embodiments, R^{7b} and R^{7c}, together with the nitrogen to which they are attached, combine to form a 3-6-membered heterocycloalkyl group. In some embodiments, R^{7b} and R^{7c}, together with the nitrogen to which they are attached, combine to form a 6-membered heterocycloalkyl group.

In some embodiments, R^{7b} and R^{7c}, together with the nitrogen to which they are attached, combine to form an azetidine. In some embodiments, R^{7b} and R^{7c}, together with the nitrogen to which they are attached, combine to form an azetidine substituted with amino.

In some embodiments, R^{7b} and R^{7c}, together with the nitrogen to which they are attached, combine to form a piperidine. In some embodiments, R^{7b} and R^{7c}, together with the nitrogen to which they are attached, combine to form a piperidine substituted with COOH.

In some embodiments, R^{7b} and R^{7c}, together with the nitrogen to which they are attached, combine to form a piperazine. In some embodiments, R^{7b} and R^{7c}, together with the nitrogen to which they are attached, combine to form an piperazine substituted with a C₁-C₃ alkyl optionally substituted with COOH. In some embodiments, R^{7b} and R^{7c}, together with the nitrogen to which they are attached, combine to form an piperazine substituted with methyl. In some embodiments, R^{7b} and R^{7c}, together with the nitrogen to which they are attached, combine to form an piperazine substituted with methyl substituted with COOH. In some embodiments, R^{7b} and R^{7c}, together with the nitrogen to which they are attached, combine to form an piperazine substituted with ethyl substituted with OH.

In some embodiments, R^{7b} and R^{7c}, together with the nitrogen to which they are attached, combine to form a morpholine.

In some embodiments, R⁷ is C₁-C₃ alkyl, C₁-C₃ haloalkyl, OR^{7a}, or

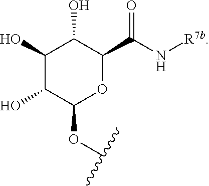

In some embodiments, R⁷ is methyl, trifluoromethyl, OR^{7a}, or

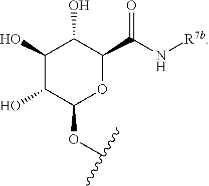

In some embodiments, R⁷ is OR^{7a} or

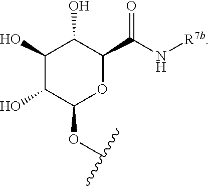

In some embodiments. R⁷ is methyl, trifluoromethyl, hydroxy, methoxy, OCH₂COOH, OCH₂CH₂NH₂, OCH₂CH₂OH, OCH₂CH₂CH₂OH,

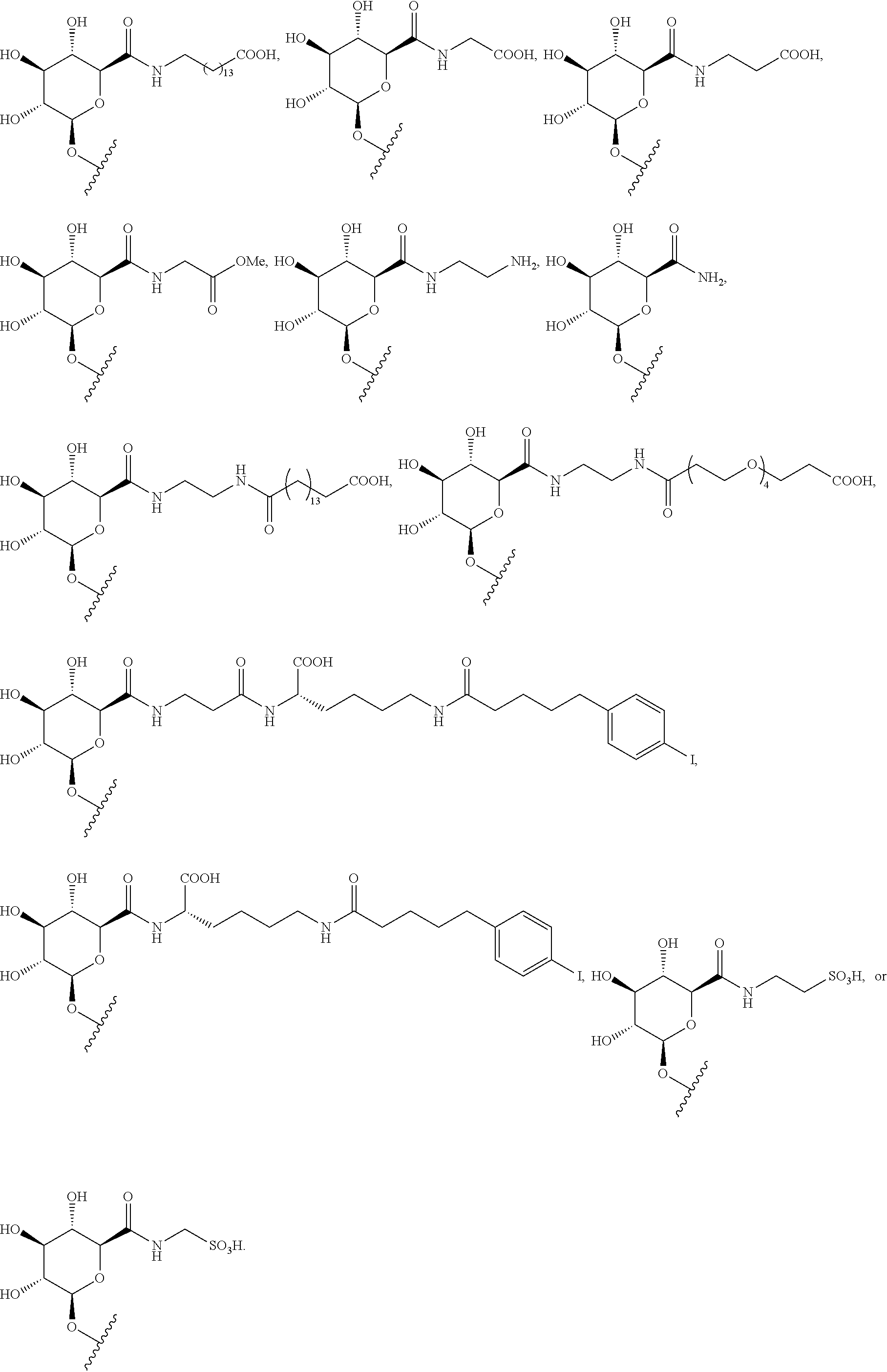

In some embodiments, R⁷ is OCH₂CH₂NH₂,

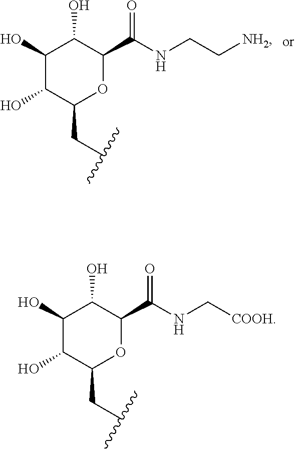 [SMILES: *C[C@@H]1O[C@H](C(=O)NCCN)[C@@H](O)[C@H](O)[C@H]1O ; *C[C@@H]1O[C@H](C(=O)NCC(=O)O)[C@@H](O)[C@H](O)[C@H]1O]

In some embodiments, R⁸ is H.

In some embodiments, R⁸ is C₁-C₃ haloalkyl.

In some embodiments, R⁸ is COOH.

In some embodiments, R⁸ is C(═O)NH—R^{8a}.

In some embodiments, R^{8a} is C₁-C₁₀ alkyl substituted with COOH or SO₃H. In some embodiments, R^{8a} is C₁-C₁₀ alkyl substituted with COOH. In some embodiments, R^{8a} is methyl substituted with COOH. In some embodiments, R^{8a} is C₁₀ alkyl substituted with COOH. In some embodiments, R^{8a} is C₁-C₁₀ alkyl substituted with SO₃H. In some embodiments, R^{8a} is ethyl substituted with SO₃H.

In some embodiments, R^{8a} is C₁-C₃ alkoxy. In some embodiments. R^{8a} is methoxy. In some embodiments, R^{8a} is C₁-C₃ alkoxy substituted with amino. In some embodiments, R^{8a} is ethoxy substituted with amino.

In some embodiments, R⁸ is

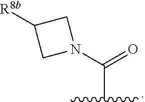

In some embodiments R⁸ is COOH. In some embodiments R^{8b} is amino.

In some embodiments, R⁸ is C(═O)NH(CH₂)₂NHC(═O)—R^{8c}. In some embodiments, R^{Q} is (CH₂CH₂O)ₓCH₂CH₂COOH. In some embodiments, R^{8c} is (CH₂)_{y}COOH. In some embodiments, x and y are as defined above.

In some embodiments, R⁸ is trifluoromethyl, COOH, C(═O)NH—R^{8a},

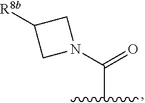

or C(═O)NH(CH₂)₂NHC(═O)—R^{8c}.

In some embodiments, R⁸ is trifluoromethyl, COOH,

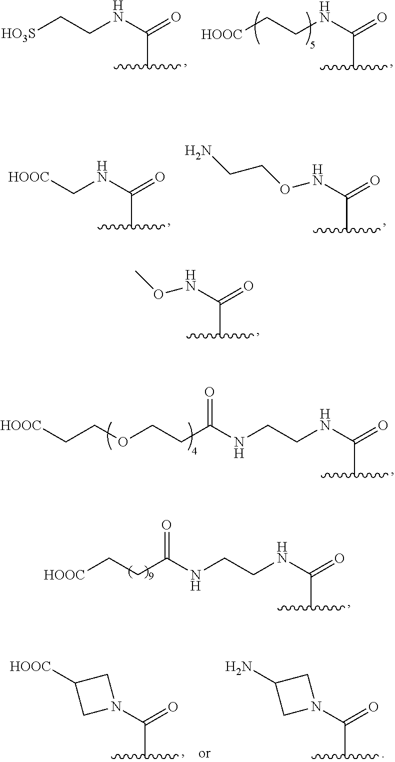

Compounds of the preset disclosure also include tautomeric forms. Tautomeric forms result from the swapping of a single bond with an adjacent double bond and the concomitant migration of a proton. Tautomeric forms include prototropic tautomers which are isomeric protonation states having the same empirical formula and total charge. Examples prototropic tautomers include ketone-enol pairs, amide-imidic acid pairs, lactam-lactim pairs, amide-imidic acid pairs, enamine-imine pairs, and annular forms where a proton can occupy two or more positions of a heterocyclic system, such as, 1H- and 3H-imidazole, 1H-, 2H- and 4H-1,2,4-triazole, 2H- and 2H-isoindole, and 2H- and 2H-pyrazole. Tautomeric forms can be in equilibrium or sterically locked into one form by appropriate substitution.

### Compounds for Making Drug Conjugates

Also disclosed herein are compounds of Formula (II) which can be used to make drug conjugates:

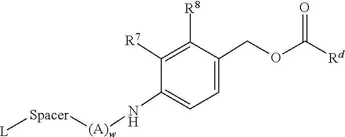

wherein:
L is a ligand;
Spacer is a spacer group;
each A is independently selected from natural or unnatural amino acids, wherein any free amine of an amino acid is optionally independently substituted with CH₃;
w is an integer selected from 1-6;
R⁷ is H, C₁-C₆ haloalkyl, OR^{7a},

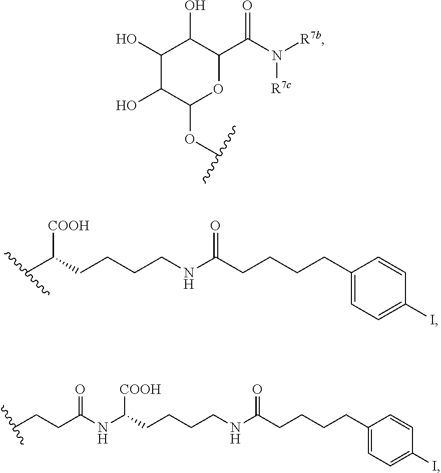

or C₁-C₆ alkyl optionally substituted with COOH;
R^{7a} is H, or C₁-C₆ alkyl optionally substituted with amino, COOH, or OH;
R^{7b} is H, C₁-C₂₀ alkyl optionally substituted with OR^{7b1}, COOR^{7b1}, NR^{7b1}₂, or SO₃R^{71b}; (CH₂)₂NHC(═O)—R^{7b2},

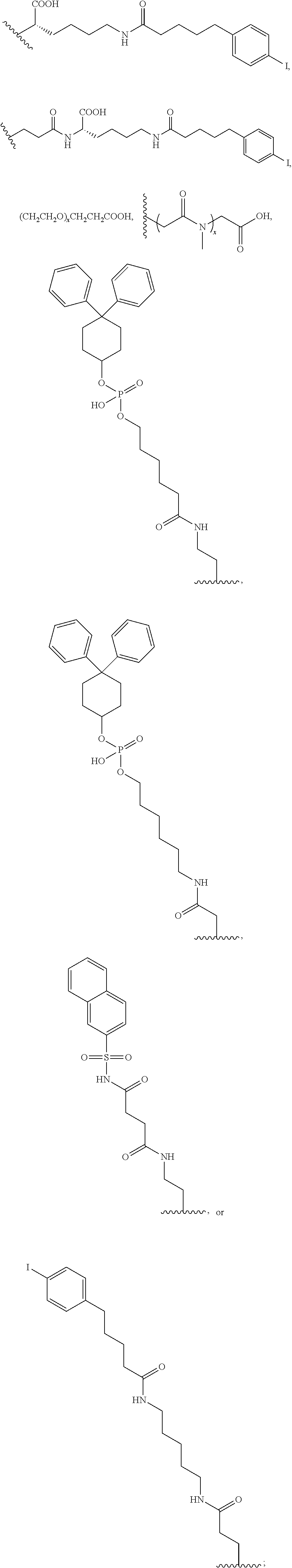

each R^{7b1} is independently selected from H and C₁-C₆ alkyl;
R^{7b2} is (CH₂CH₂O)ₓCH₂CH₂COOH, or (CH₂)_{y}COOH;
R^{7c} is H or C₁-C₆ alkyl; or
R^{7b} and R^{7c}, together with the nitrogen to which they are attached, combine to form a heterocycloalkyl group optionally substituted with amino, COOH, C₁-C₆ alkyl which is optionally substituted with OH or COOH;
R⁸ is H, C₁-C₆ haloalkyl, COOH, C(═O)NH—R^{8a},

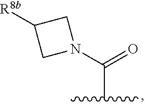

or C(═O)NH(CH₂)₂NHC(═O)—R^{8c};
R^{8a} is C₁-C₂₀ alkyl substituted with COOH or SO₃H, or C₁-C₆ alkoxy optionally substituted with amino;
R^{8b} is COOH or amino;
R^{8c} is (CH₂CH₂O)ₓCH₂CH₂COOH or (CH₂)_{y}COOH;
each x is independently an integer selected from 1-10;
each y is independently an integer selected from 1-20;
R^{d} is the residue of a bioactive molecule of the formula R^{d}—H;
wherein when R⁷ is H, then R^{R} is C₁-C₆ haloalkyl, COOH, C(═O)NH—R^{8a},

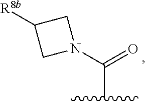

or C(═O)NH(CH₂)₂NHC(═O)—R^{8c}; and
wherein when R⁸ is H, then R⁷ is C₁-C₆ haloalkyl, OR^{7a}

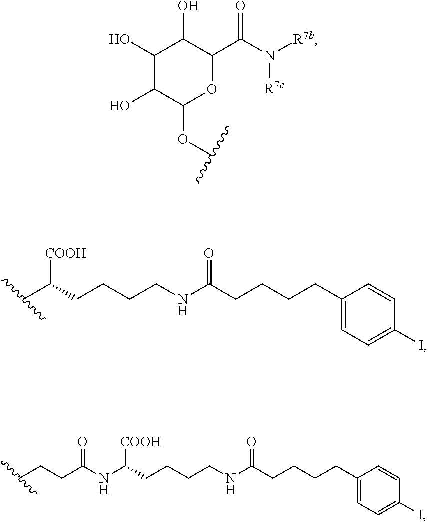

or C₁-C₆ alkyl optionally substituted with COOH

In some embodiments, R⁷ is H, C₁-C₃ haloalkyl, OR^{7a},

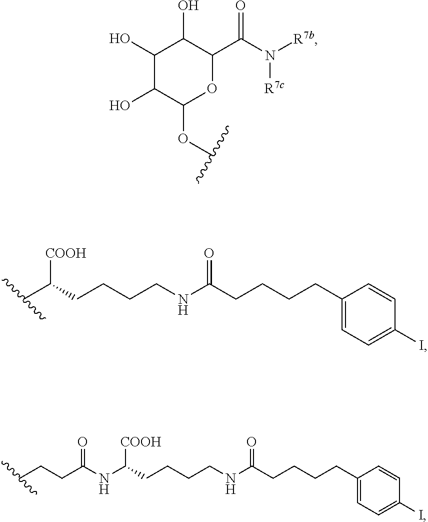

or C₁-C₃ alkyl optionally substituted with COOH;

R^{7a} is H, or C₁-C₃ alkyl optionally substituted with amino, COOH, or OH;
H, C₁-C₁₄ alkyl optionally substituted with OR^{7b1}, COOR^{7b1}, NR^{7b1}₂, or SO₃R^{7b1}; (CH₂)₂NHC(═O)—R^{7b2},

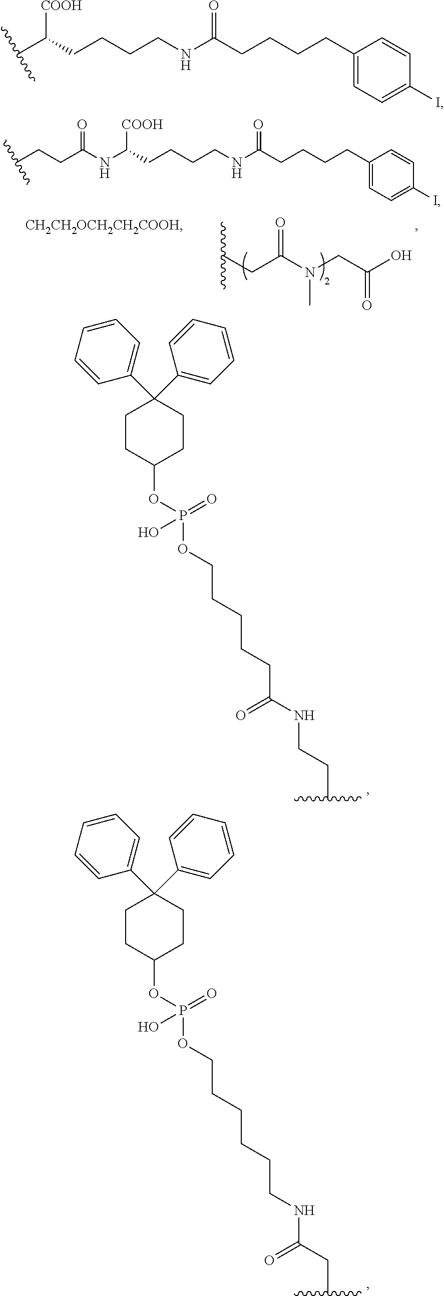

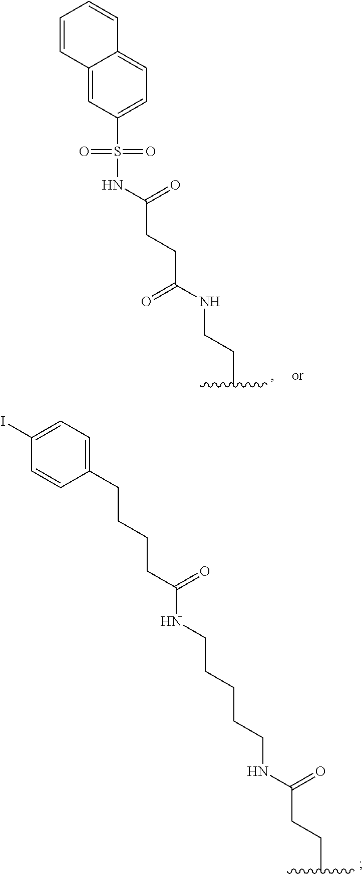 [SMILES: *CCNC(=O)CCC(=O)NS(=O)(=O)c1ccc2ccccc2c1 ; *CCC(=O)NCCCCCNC(=O)CCCCc1ccc(I)cc1]

R^{7b1} is H or C₁-C₃ alkyl;
R^{7b2} is (CH₂CH₂O)ₓCH₂CH₂COOH or (CH₂)_{y}COOH,
R^{7c} is H or C₁-C₃ alkyl; or
R^{7b} and R^{7c}, together with the nitrogen to which they are attached, combine to form a 3-6-membered heterocycloalkyl group optionally substituted with amino, COOH, or C₁-C₃ alkyl which is optionally substituted with OH or COOH;
R⁸ is H, C₁-C₃ haloalkyl, COOH, C(═O)NH—R^{8a},

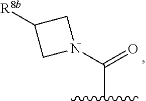

or C(═O)NH(CH₂)₂NHC(═O)—R^{8c};
R^{8a} is C₁-C₁₀ alkyl substituted with COOH or SO₃H, or C₁-C₃ alkoxy optionally substituted with amino;
R^{8b} is COOH or amino;
R^{8c} is (CH₂CH₂O)ₓCH₂CH₂COOH or (CH₂)_{y}COOH;
wherein when R⁷ is H, then R⁸ is C₁-C₃ haloalkyl, COOH, C(═O)NH—R^{8a},

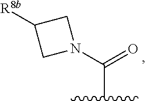

or C(═O)NH(CH₂)₂NHC(═O)—R^{8c}; and
wherein when R⁸ is H, then R⁷ is C₁-C₃ haloalkyl, OR^{7a},

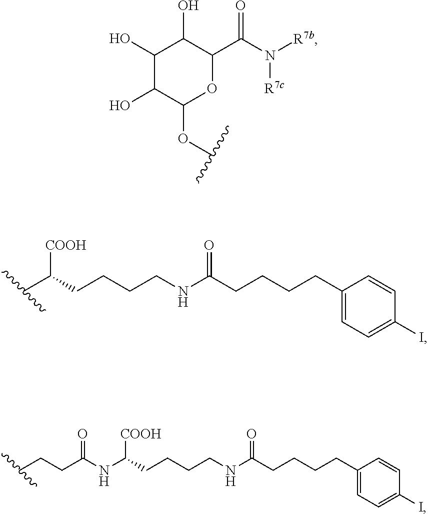

or C₁-C₃ alkyl optionally substituted with COOH.

In some embodiments, disclosed herein are compounds of Formula (II) wherein:
L is a ligand;
Spacer is a spacer group;
each A is independently selected from natural or unnatural amino acids, wherein any free amine of an amino acid is optionally independently substituted with CH₃;
w is an integer selected from 1-6;
R⁷ is H, C₁-C₆ alkyl, C₁-C₆ haloalkyl, OR^{7a}, or

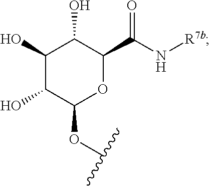

R^{7a} is H, or C₁-C₆ alkyl optionally substituted with amino, COOH, or OH;
R^{7b} is H; C₁-C₂₀ alkyl substituted with COOR^{7b1}, amino or SO₃H; (CH₂)₂NHC(═O)—R^{7b2};

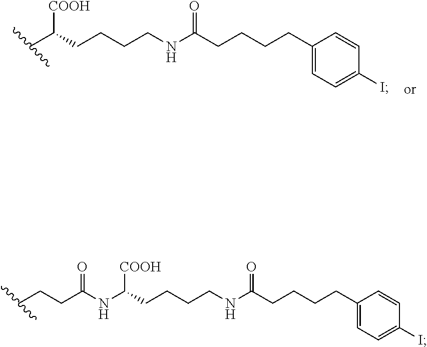

R^{7b1} is H or C₁-C₆ alkyl;
R^{7b2} is (CH₂CH₂O)ₓCH₂CH₂COOH or (CH₂)_{y}COOH;
R⁸ is H, C₁-C₆ haloalkyl, COOH, C(═O)NH—R^{8a},

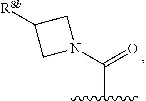

or C(═O)NH(CH₂)₂NHC(═O)—R^{8c};
R^{8a} is C₁-C₂₀ alkyl substituted with COOH or SO₃H; or C₁-C₆ alkoxy optionally substituted with amino;
R^{8b} is COOH or amino;
R^{8c} is (CH₂CH₂O)ₓCH₂CH₂COOH or (CH₂)_{y}COOH;
each x is independently an integer selected from 1-10;
each y is independently an integer selected from 1-20;
R^{d} is the residue of a bioactive molecule of the formula R^{d}—H;
wherein when R⁷ is H, then R⁸ is C₁-C₆ haloalkyl, COOH, C(═O)NH—R^{8a},

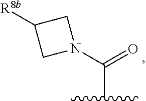

or C(═O)NH(CH₂)₂NHC(═O)—R^{8c}; and
wherein when R⁸ is H, then R⁷ is C₁-C₆ alkyl, C₁-C₆ haloalkyl, OR^{7a}, or

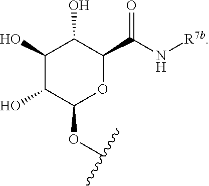

In some embodiments, R⁷ is H, C₁-C₃ alkyl, C₁-C₃ haloalkyl, OR^{7a}, or

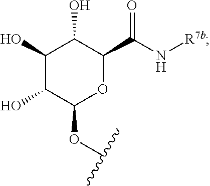

R^{7a} is H, or C₁-C₃ alkyl optionally substituted with amino, COOH, or OH;
R^{7b} is H; C₁-C₁₄ alkyl substituted with COOR^{7b1}, amino, or SO₃H; (CH₂)₂NHC(═O)—R^{7b2};

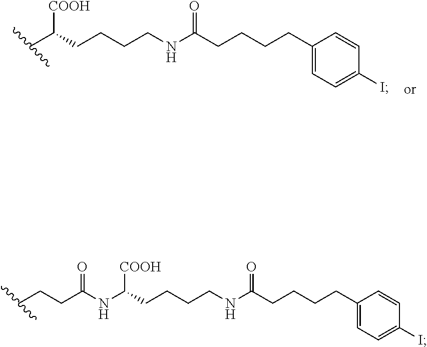

R^{7b1} is H or C₁-C₃ alkyl;
R^{7b2} is (CH₂CH₂O)ₓCH₂CH₂COOH, or (CH₂)_{y}COOH;
R⁸ is H, C₁-C₃ haloalkyl, COOH, C(═O)NH—R^{8a},

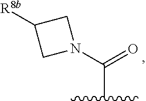

or C(═O)NH(CH₂)₂NHC(═O)—R^{8c};
R^{8a} is C₁-C₁₀ alkyl substituted with COOH or SO₃H; or C₁-C₃ alkoxy optionally substituted with amino;
R^{8b} is COOH or amino;
R^{8c} is (CH₂CH₂O)ₓCH₂CH₂COOH or (CH₂)_{y}COOH;
wherein when R⁷ is H, then R⁸ is C₁-C₃ haloalkyl, COOH, C(═O)NH—R^{8a},

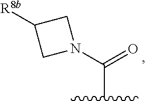

or C(═O)NH(CH₂)₂NHC(═O)—R^{8c}; and
wherein when R⁸ is H, then R⁷ is C₁-C₃ alkyl, C₁-C₃ haloalkyl, OR^{7a}, or

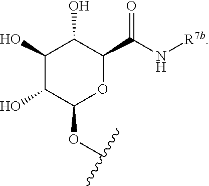

In some embodiments, R⁷ is methyl, trifluoromethyl, OR^{7a}, or

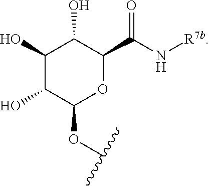

In some embodiments, R⁷ is

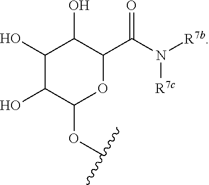

In some embodiments, R⁷ is OR^{7a} or

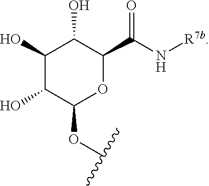

In some embodiments. R⁷ is methyl, trifluoromethyl, hydroxy, methoxy, OCH₂COOH, OCH₂CH₂NH₂, OCH₂CH₂OH, OCH₂CH₂CH₂OH,

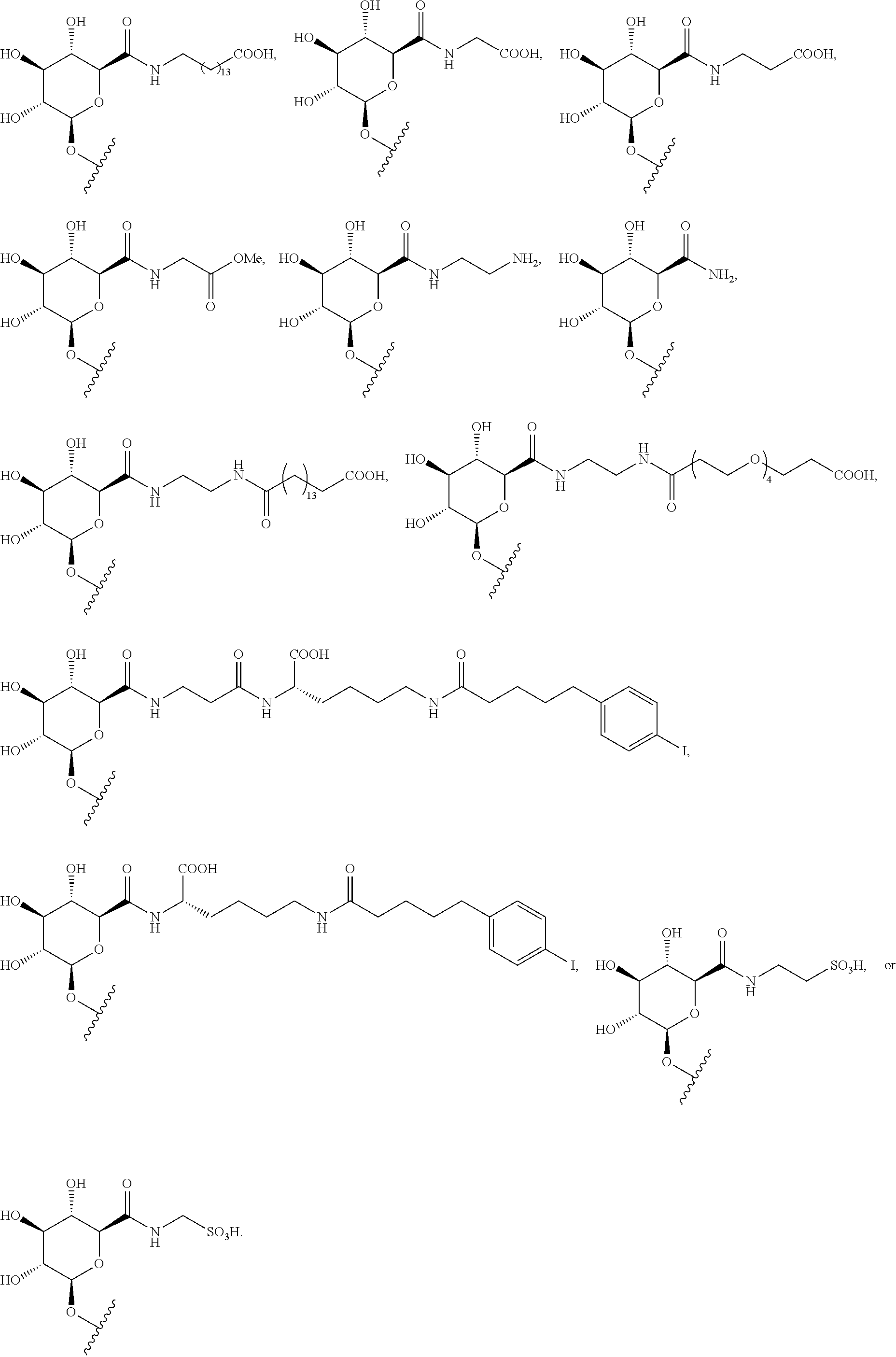

In some embodiments, R⁷ is OCH₂CH₂NH₂,

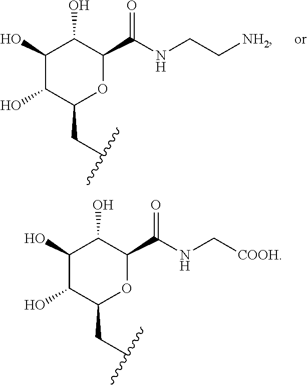 [SMILES: *C[C@@H]1O[C@H](C(=O)NCCN)[C@@H](O)[C@H](O)[C@H]1O ; *C[C@@H]1O[C@H](C(=O)NCC(=O)O)[C@@H](O)[C@H](O)[C@H]1O]

In some embodiments, R⁸ is trifluoromethyl, COOH, C(═O)NH—R^{8a},

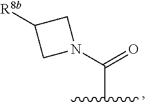

or C(═O)NH(CH₂)₂NHC(═O)—R^{8c}.

In some embodiments, R⁸ is trifluoromethyl, COOH

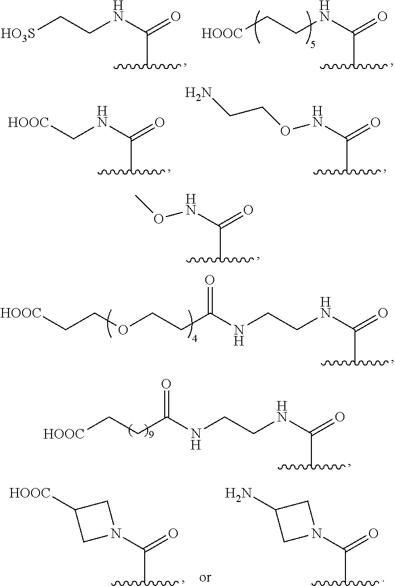

In some embodiments, R⁷ and R⁸ are as defined for compounds of Formula (I) above.

In some embodiments, L is a ligand. Ligands include, but are not limited to, small molecules. In some embodiments, the ligand, L, can contain a functional group that allows for conjugation to an antibody. Examples of functional groups that allow for conjugation to an antibody include, but are not limited to, an amino group (NH₂).

### Bioactive Molecules and Residues Thereof

In some embodiments, R^{d} is the residue of a bioactive molecule of the formula R^{d}—H.

In some embodiments, R^{d}—H is a chemotherapeutic that is a cytotoxic drug, kinase inhibitor or both.

In some embodiments, R^{d}—H is a chemotherapeutic that is a ligand for an extracellular protein in the extracellular environment of the tumor cells expressing the GPCR.

In some embodiments. R^{d}—H is a cytotoxic drug that is an antimitotic, DNA-damaging agent, transcriptional inhibitor or combination thereof.

In some embodiments, R^{d}—H is a cytotoxic drug that is an antimitotic, and wherein the antimitotic is a maytansinoid, taxane, auristatin, alkaloid, tubulysin, or epothilone.

In some embodiments, R^{d}—H is a cytotoxic drug that is a DNA-damaging agent, transcriptional inhibitor, or combination thereof, wherein the DNA-damaging agent, transcriptional inhibitor, or combination thereof is a DNA polymerase inhibitor, DNA replication inhibitor, topoisomerase inhibitor, cytotoxic antibiotic.

In some embodiments, R^{d}—H is a kinase inhibitor that is an inhibitor of a cytoplasmic tyrosine kinase (CTK), a serine/threonine kinase (S/T Kinase), a lipid kinase (LK), or a receptor tyrosine kinase (RTK).

In some embodiments. R^{d}—H is a cytotoxic drug that is an antimitotic, DNA-damaging agent, transcriptional inhibitor or combination thereof. In some embodiments, R^{d} comprises a cytotoxic drug that is an antimitotic, and wherein the antimitotic is a maytansinoid, taxane, auristatin, alkaloid, tubulysin, or epothilone. In some embodiments, the maytansinoid is mertansine or emtansine; the taxane is paclitaxel or docetaxel; the alkaloid is ellipticine or a vinca alkaloid; the Epothilone is epothilone A, epothilone B, epothilone C, epothilone D, epothilone E, epothilone F, or utidelone; the auristatin is monomethyl auristatin E or monomethyl auristatin F.

In some embodiments, the vinca alkaloid is vinblastine, vincristine, vindesine, vinorelbine, or vinflunine.

In some embodiments, the DNA-damaging agent, transcriptional inhibitor, or combination thereof is a DNA polymerase inhibitor. DNA replication inhibitor, topoisomerase inhibitor, cytotoxic antibiotic.

In some embodiments, the DNA polymerase inhibitor is amikhelline, aphidicolin, cytarabine, vernolepin; the DNA replication inhibitor is altretamine, bleomycin, cytarabine, dacarbazine, dactinomycin, ellipticine, estramustine, mitobronitol, mitomycin, mitosene, pingyangmycin, plicamycin, procarbazine, or temozolomide; the topoisomerase inhibitor is irinotecan, topotecan, camptothecin, etoposide, doxorubicin, mitoxantrone, teniposide, novobiocin, merbarone, or aclarubicin; the cytotoxic antibiotic is an anthracycline, bleomycin, mitomycin, or actinomycin.

In some embodiments, the anthracycline is doxorubicin, daunorubicin, epirubicin, idarubicin, pirarubicin, aclarubicin, or mitoxantrone; the mitomycin is mitomycin A, mitomycin B, or mitomycin C.

In some embodiments, a cytotoxic drug is selected from actinomycin, aldesleukin, alemtuzumab, alitretinoin, altretamine, amsacrine, anastrozole, azacitidine, azathioprine, bevacizumab, bexarotene, bicalutamide, bleomycin, bortezomib, botulinum toxin, busulfan, capecitabine, carboplatin, carmustine, cetuximab, clorambucil, chloramphenicol, chlormethine hydrochloride, ciclosporin, cidofovir, cisplatin, cladribine, clofarabine, clorambucil, crisantaspase, cyclophosphamide, cytarabine, dacarbazine, dactinomycin, danazol, dasatinib, daunorubicin hydrochloride, decitabine, denileukin, dienostrol, diethylstilbestrol, dinoprostone, docetaxel, doxorubicin, dutasteride, epirubicin, estradiol, estramustine phosphate sodium, estrogen-progestin combinations, estrogens conjugated, estrogens esterified, estrone, estropipate, etoposide, exemestane, finasteride, floxuridine, fludarabine, fluorouracil, fluoxymesterone, flutamide, fulvestrant, ganciclovir, gemcitabine, ibritumomab tiuxetan, idarubicin, ifosfamide, imatinib mesilate, irinotecan hydrochloride, leflunomide, letrozole, leuprorelin acetate, lomustine, lymphoglobuline, medroxyprogesterone, megestrol, melphalan, mercaptopurine, mesena, methotrexate, methyltestosterone, mifepristone, mitomycin, mitotane, mitoxantrone hydrochloride, mycophenolate mofetil, nafarelin, natalizumab, nilutamide, oestrogen, oxaliplatin, oxytocin (including syntocinon and syntometrine), paclitaxel, pemetrexed disodium, pentamidine isethionate, pentostatin, perphosphamide, pipobroman, piritrexim isethionate, plicamycin, podoflilox, prednimustine, procarbazine, progesterone containing products, progestins, raloxifene, raltitrexed, ribavirin, rituximab, sirolimus, streptozocin, tacrolimus, tamoxifen, temozolomide, teniposide, testolactone, testosterone, thalidomide, thioguanine, thiotepa, thymoglobulin, tioguanine, topotecan, toremifene citrate, tositumomab, trastuzumab, treosulfan, tretinoin, trifluridine, trimetrexate glucuronate, triptorelin, uramustine, valganciclovir, valrubicin, vidarabine, vinblastine sulfate, vincristine, vindesine, vinorelbine tartrate, and zidovudine.

In some embodiments. R^{d}—H is a kinase inhibitor. In some embodiments, R^{d}—H is a kinase inhibitor that is an inhibitor of a cytoplasmic tyrosine kinase (CTK), a serine/threonine kinase (S/T Kinase), a lipid kinase (LK), or a receptor tyrosine kinase (RTK).

In some embodiments, the cytoplasmic tyrosine kinase (CTK) is Sphingosine kinase 1 (SK1), or phosphoinositide 3-kinase (PI3K).

In some embodiments, the serine/threonine kinase (S/T Kinase) is Protein kinase Ci (PKCi), mammalian target of rapamycin (mTOR), cyclin-dependent kinases (CDKs), Ataxia telangiectasia mutated (ATM), protein kinase B (Akt), ribosomal protein S6 kinase (S6K), Serine/threonine kinase 11 or liver kinase B1 (STK11/LKB1), Polo-like kinases (PLKs), B-Raf proto-oncogene (b-Raf), Aurora Kinase A & B (Aur A & B), Mitogen Activated Protein (MAP) kinase (MAP3K), or Mitogen-activated protein kinase (MEK),

In some embodiments, the lipid kinase (LK) is Proto-oncogene tyrosine-protein kinase Src (c-SRC), c-Yes proto-oncogene (c-YES), Abelson murine leukemia viral oncogene homolog 1 (Abl), or Janus kinase 2 (JAK-2).

In some embodiments, the receptor tyrosine kinase (RTK) is Recepteur d'Origine Nantais (RON), Fibroblast growth factor receptors (FGFRs), c-MET proto-oncogene (c-Met), c-RET proto-oncogene(c-Ret), Insulin-like growth factor 1 receptor (IGF-IR), Epidermal growth factor receptor (EGFR), Platelet-derived growth factor receptor α (PDGFR-α), proto-oncogene c-Kit or Mast/stem cell growth factor receptor (c-Kit), Fins-like tyrosine kinase 3, 4 (Flt3,Flt-4), Platelet-derived growth factor receptor β (PDGFR-β), Anaplastic lymphoma kinase (ALK), human epidermal growth factor receptor-2 (HER-2), or Bruton's tyrosine kinase (BTK).

In some embodiments. R^{d}—H is acalabrutinib, afatinib, alectinib, axitinib, brigatinib, bosutinib, ceritinib, crizotinib, cabozantinib, dasatinib, dabrafenib, erlotinib, gefitinib, ibrutinib, imatinib, lapatinib, lenvatinib, nilotinib, nintedanib, osimertinib, palbociclib, pazopanib, ponatinib, regorafenib, ribociclib, ruxolitinib, sorafenib, sunitinib, trametinib, vandetanib, vemurafenib, or zanubrutinib.

In some embodiments, R^{d} is:

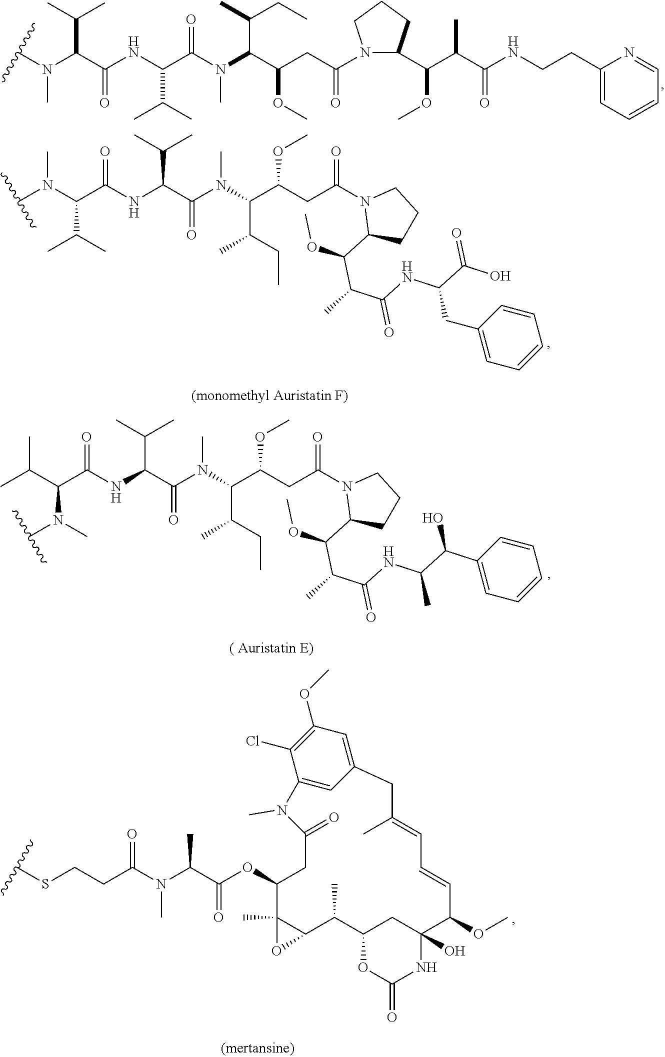

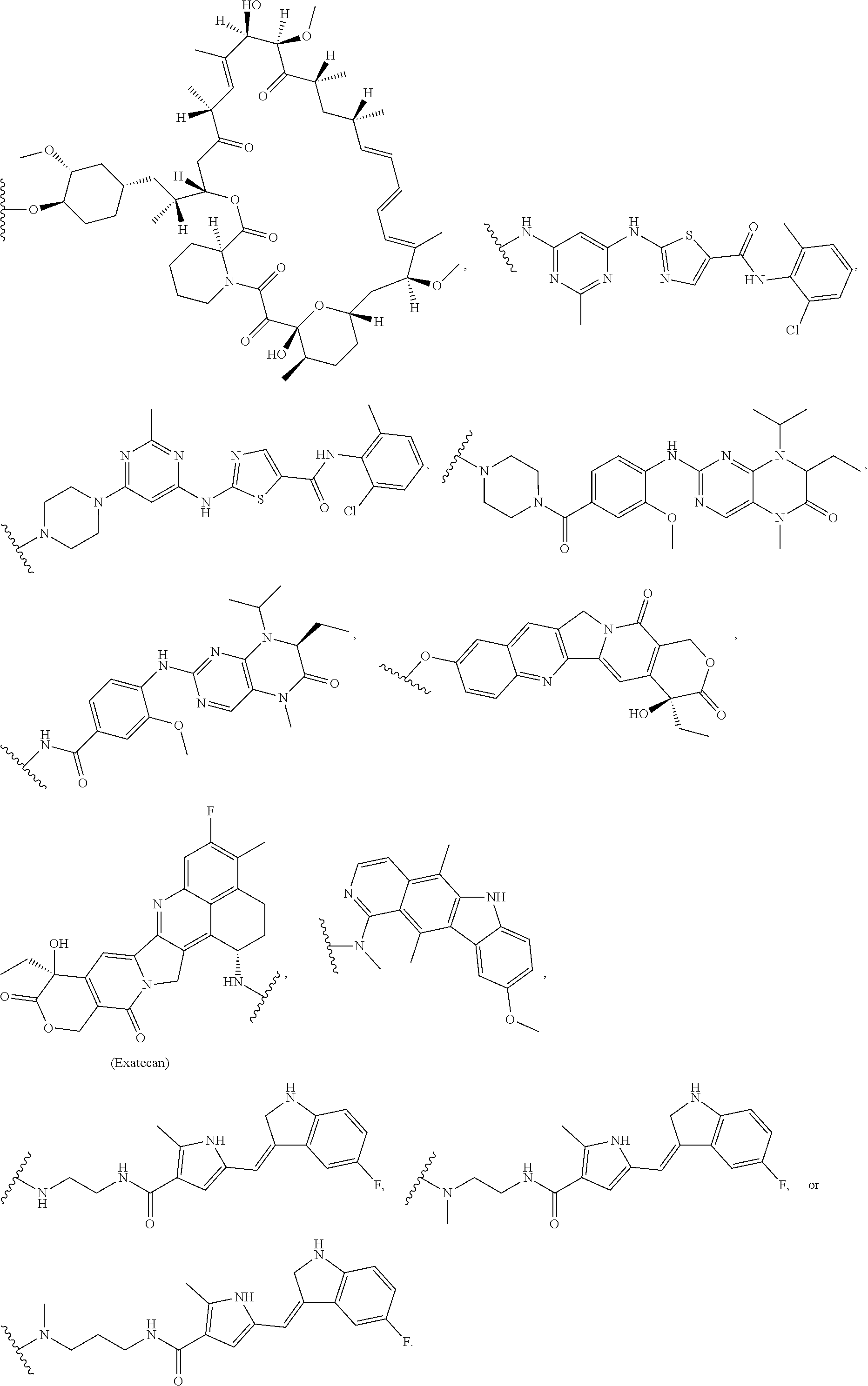

In some embodiments, R^{d}—H is an auristatin. In some embodiments, R^{d}—H is monomethyl auristatin E (auristatin E).

### Spacers

In some embodiments, the NDC of Formula (I) contains a Spacer, which is a spacer group. In some embodiments, the spacer group has a prescribed length thereby linking the ligand and R^{d} or linker/R^{d} while allowing an appropriate distance there between. In some embodiments, the spacer group is between 10 Å and 100 Å in length. In some embodiments, the spacer group has between 5 and 50 linear atoms, preferable atoms selected from C, N, and O. In some embodiments, the spacer group is between 100 and 1000 Da in mass. In some embodiments, the spacer group is between 5-10 linear atoms, selected from C, N, and O. In some embodiments, the spacer group is 7 linear atoms, selected from C and O. In some embodiments, the spacer group is 10 linear atoms, selected from C, N, and O.

In some embodiments, the spacer group includes one or more carbonyl groups. In some embodiments, the spacer group includes two carbonyl groups. In some embodiments, the spacer group includes an ether group. In some embodiments, the spacer group includes an amido group. In some embodiments, the spacer group includes two carbonyl groups and one ether group. In some embodiments, the spacer group includes two carbonyl groups, one amido group, and one ether group.

The spacer can also modulate the pharmacological activity of the NDC.

In some embodiments, the Spacer is a spacer group that is:

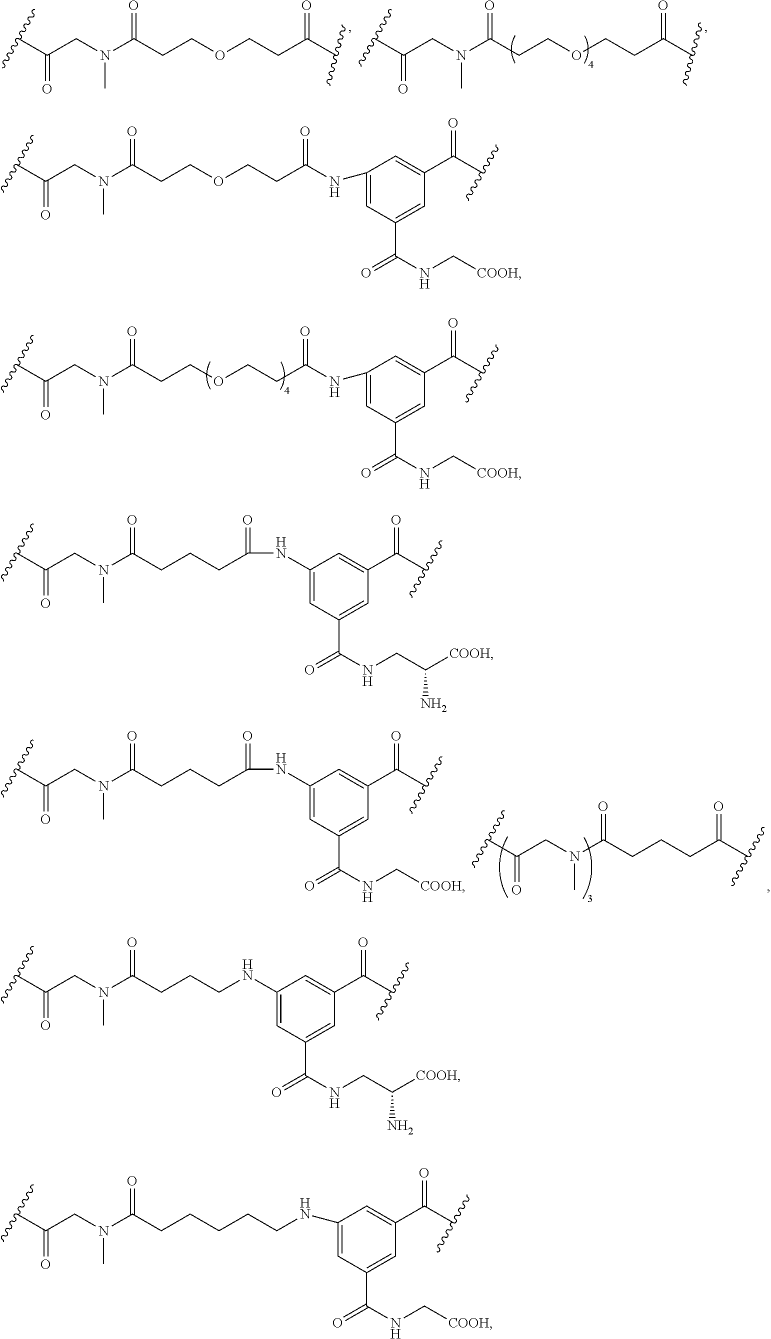

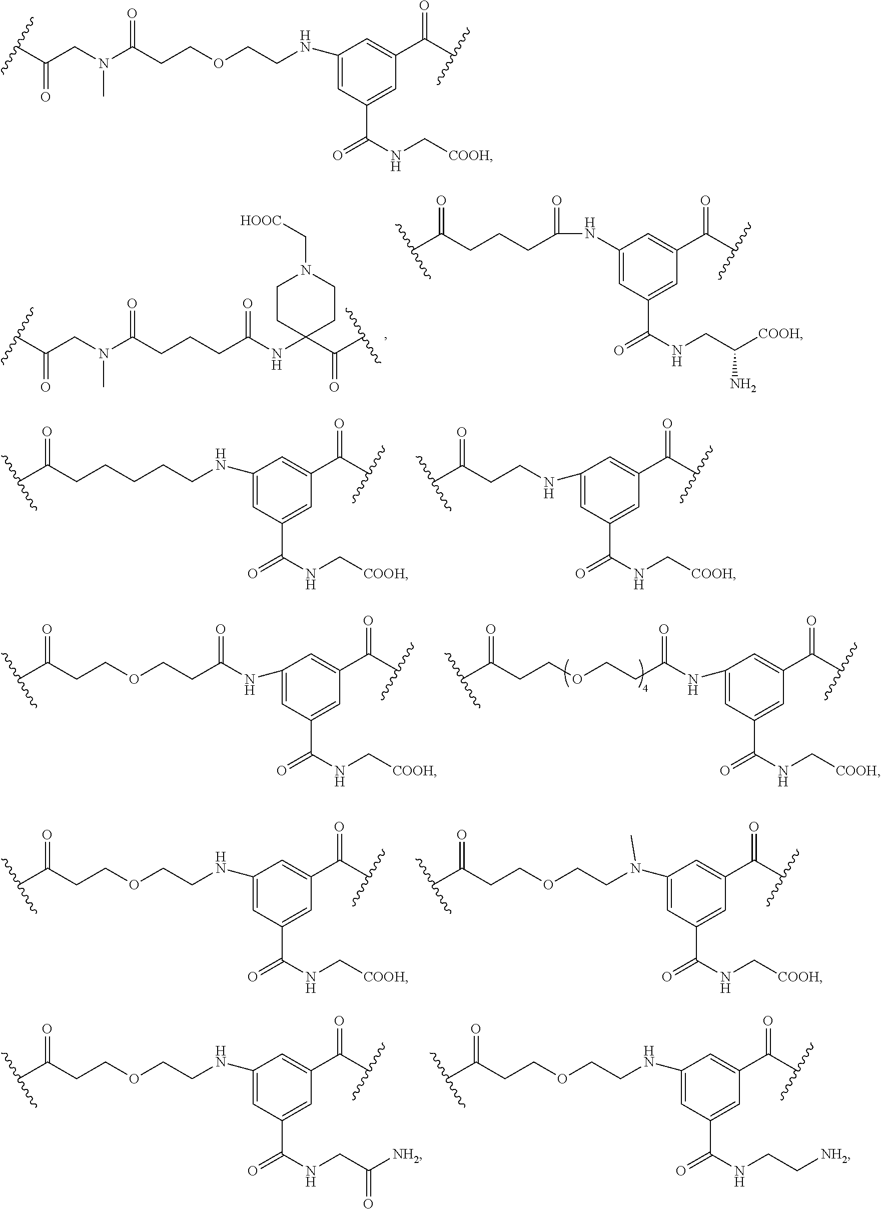

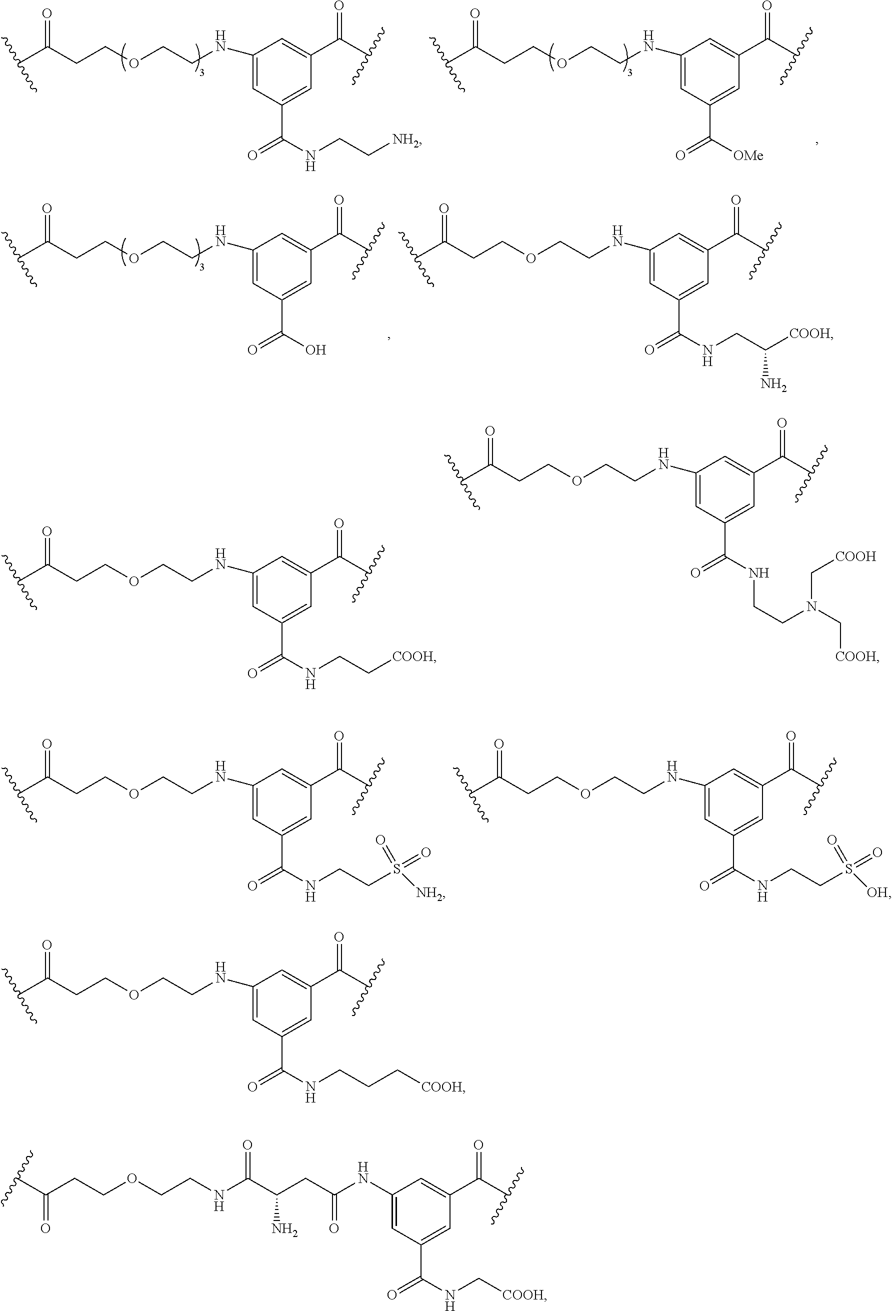

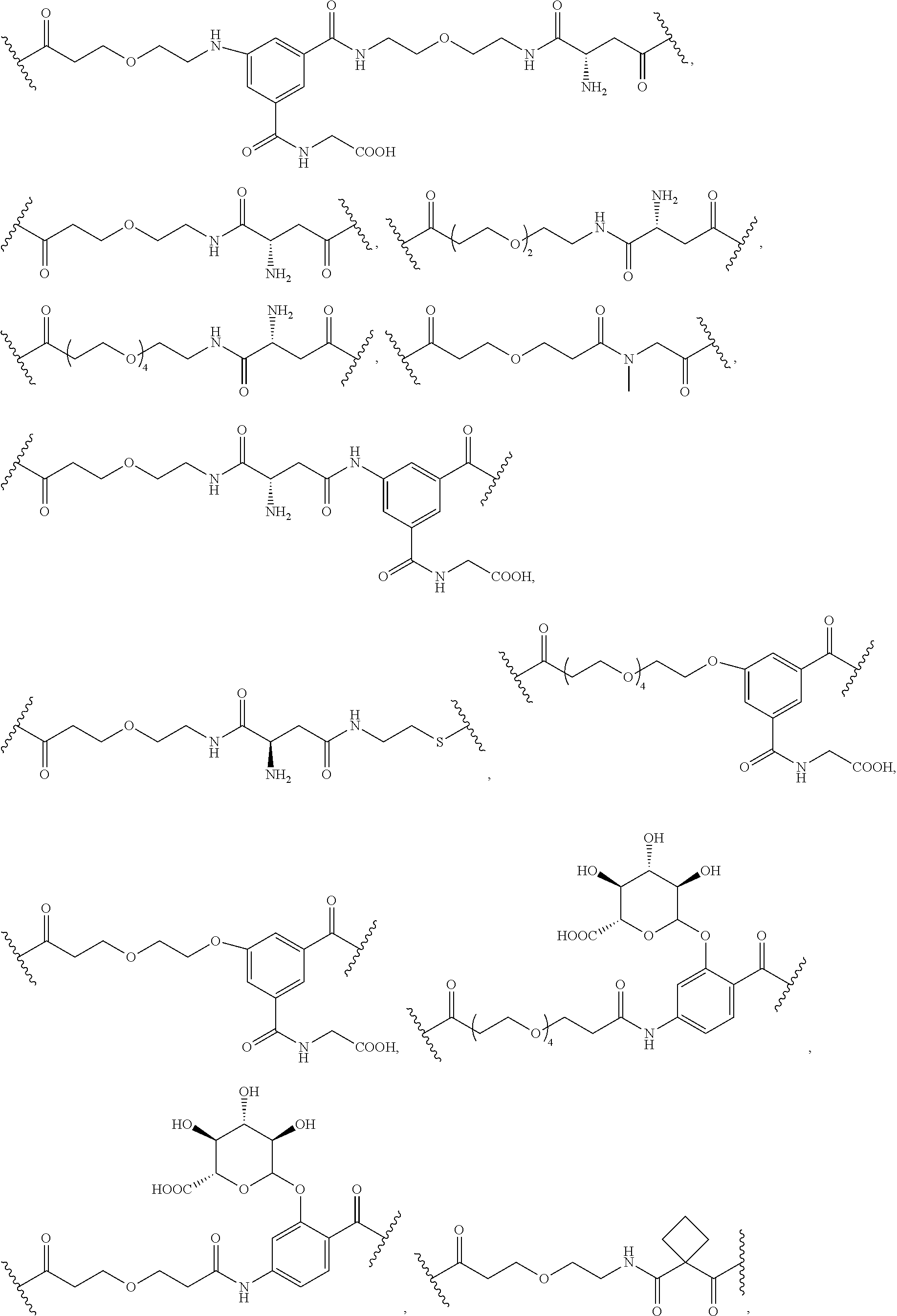

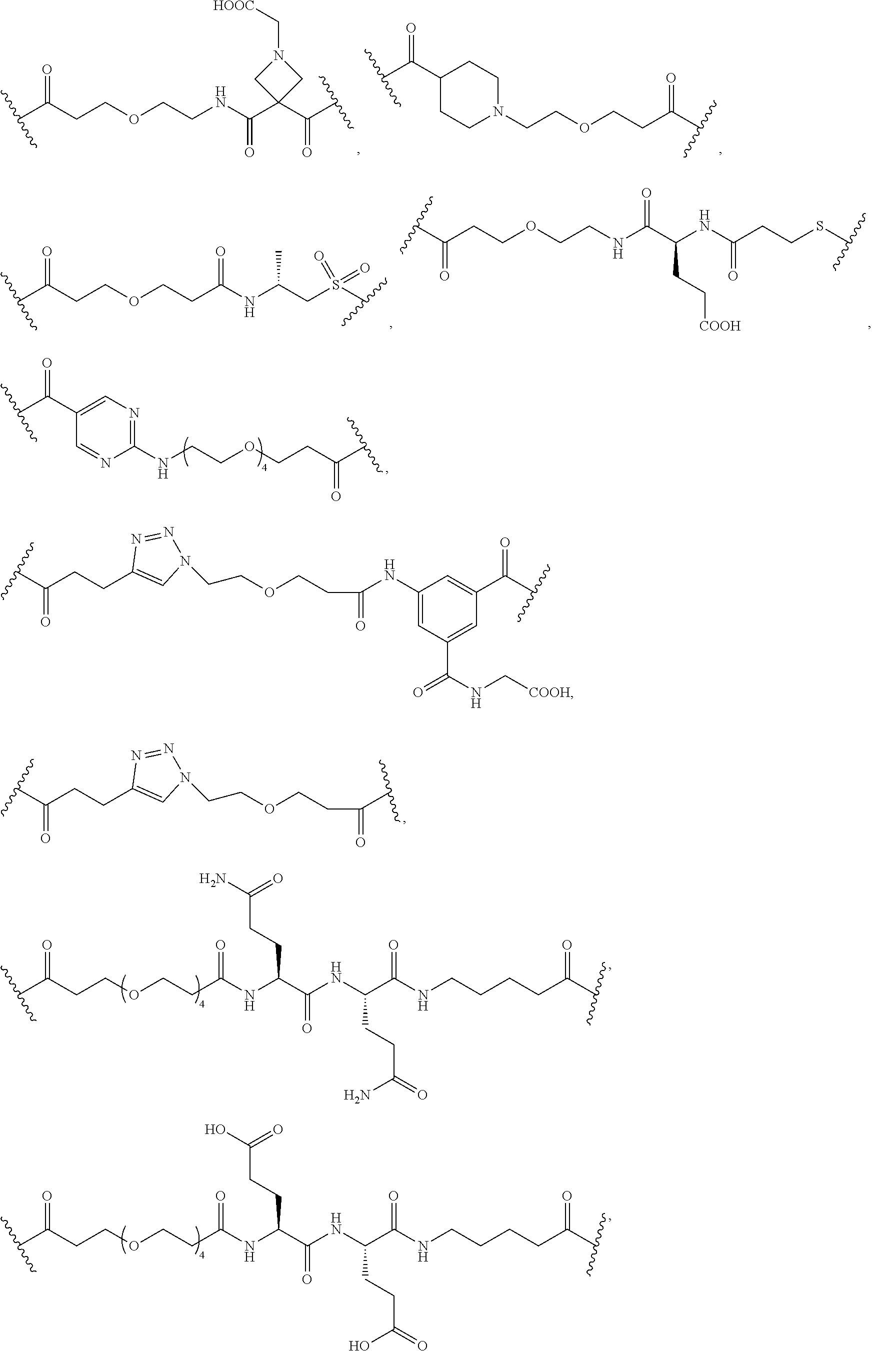

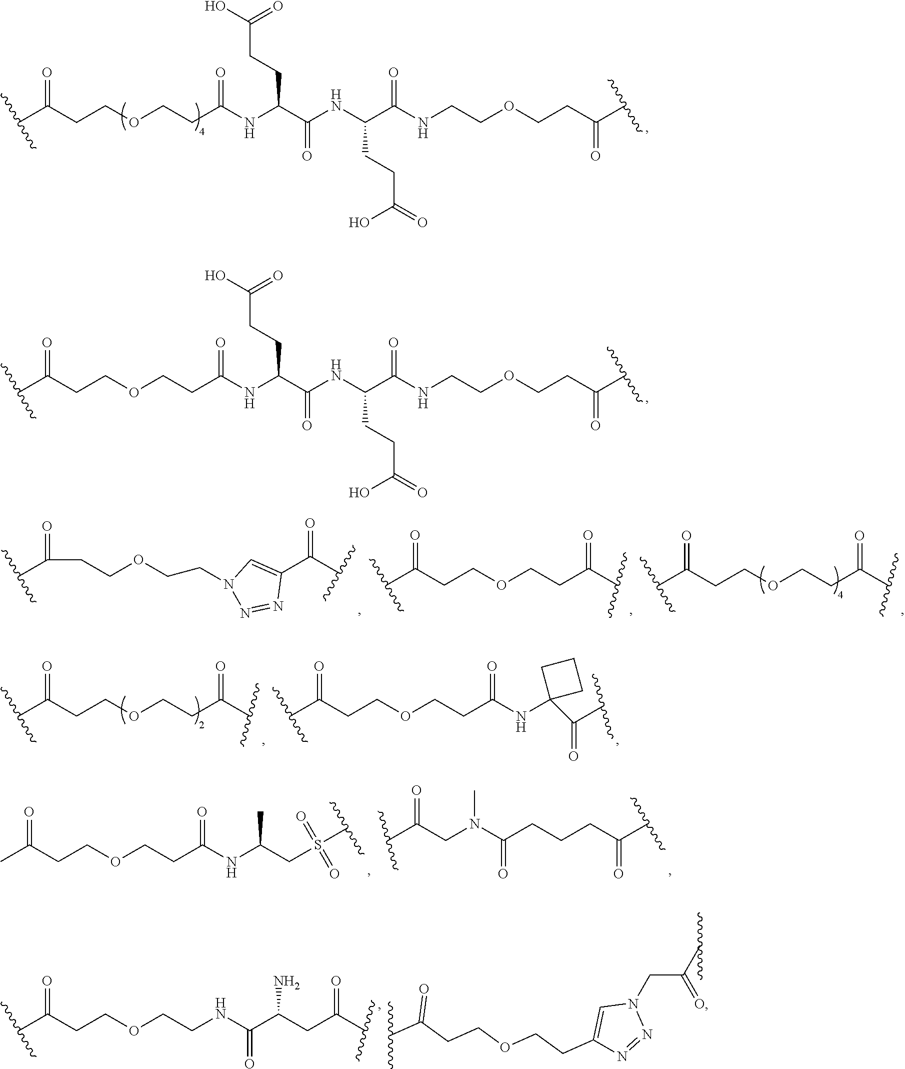

In some embodiments, the Spacer is a spacer group that is

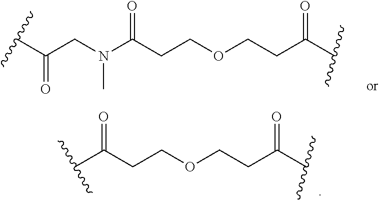

### Linkers

A linker is incorporated between the spacer group (Spacer) and the residue of a bioactive molecule (R^{d}) and comprises a peptide linkage. The peptide linkage comprises L-amino acids and/or D-amino acids. In some embodiments, D-amino acids are preferred in order to minimize immunogenicity and nonspecific cleavage by background peptidases or proteases. Cellular uptake of oligo-D-arginine sequences is known to be as good as or better than that of oligo-L-arginine.

A linker unit may be “self-immolative.” A “self-immolative” linker unit allows for release of the drug moiety without a separate hydrolysis step.

The linker comprises a p-aminobenzyl unit. In one such embodiment, a p-aminobenzyl alcohol is attached to an amino acid unit via an amide bond, and a carbamate, methylcarbamate, or carbonate is made between the benzyl alcohol and a cytotoxic agent. In one embodiment, the linker comprises p-aminobenzyloxycarbonyl (PABC).

In some embodiments, the linker is cleavable. In some embodiments, a linker is designed for cleavage in the presence of particular conditions or in a particular environment, such conditions or environments near such targeted cells, tissues, or regions. Cleavable linkers rely on the inherent properties of a cell's cytoplasmic compartments for selective release of the cytotoxic drug.

In some embodiments, the linker is cleaved by an intracellular peptidase or protease enzyme, including, but not limited to, a lysosomal or endosomal protease. In some embodiments, the linker is cleaved by a glycosidase, e.g., glucuronidase. Small peptide sequences such as Val-Cit and Phe-Lys have been developed as linkers for ADCs. These bi-peptide linkers show good stability in serum, yet can be recognized and rapidly hydrolyzed by certain lysosomal proteases, such as cathepsin B, following internalization. β-glucuronide linkers can be readily cleaved by the abundant lysosomal enzyme β-glucuronidase, facilitating facile and selective release of the active drug.

In some embodiments, the linker is cleaved by a protease, a matrix metalloproteinase, a serine protease, or a combination thereof. In some embodiments, the linker is cleaved by a reducing agent. In some embodiments, the linker is cleaved by an oxidizing agent or oxidative stress.

In some embodiments, the linker is cleaved by a matrix metalloproteinases (MMP). The hydrolytic activity of matrix metalloproteinases (MMPs) has been implicated in the invasive migration of metastatic tumor cells.

In some embodiments, the linker is cleaved by proteolytic enzymes or reducing environment, as may be found near cancerous cells. Such an environment, or such enzymes, are typically not found near normal cells.

In some embodiments, the linker is cleaved by serine proteases including but not limited to thrombin and cathepsins. In some embodiments, the linker is cleaved by cathepsin K, cathepsin S, cathepsin D, cathepsin E, cathepsin W, cathepsin F, cathepsin A, cathepsin C, cathepsin H, cathepsin Z, or any combinations thereof. In some embodiments, the linker is cleaved by cathepsin K and/or cathepsin S.

In some embodiments, the linker is a cleavable linker.

In some embodiments, linker is a cleavable linker that is a protease sensitive linker or glutathione-sensitive linker.

The linker of Formula (I) and (II) of the present invention is

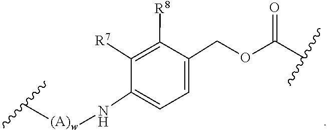

The inventors have found that introduction of the R⁷ or R⁸ substituents (as defined above) to the linker helps the self-immolitive moiety retain its stability in human and mouse serum while still allowing for efficient enzyme cleavage.

In some embodiments, the linker,

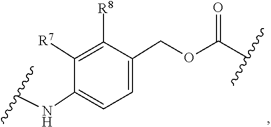

### of Formula (I) and (II) is:

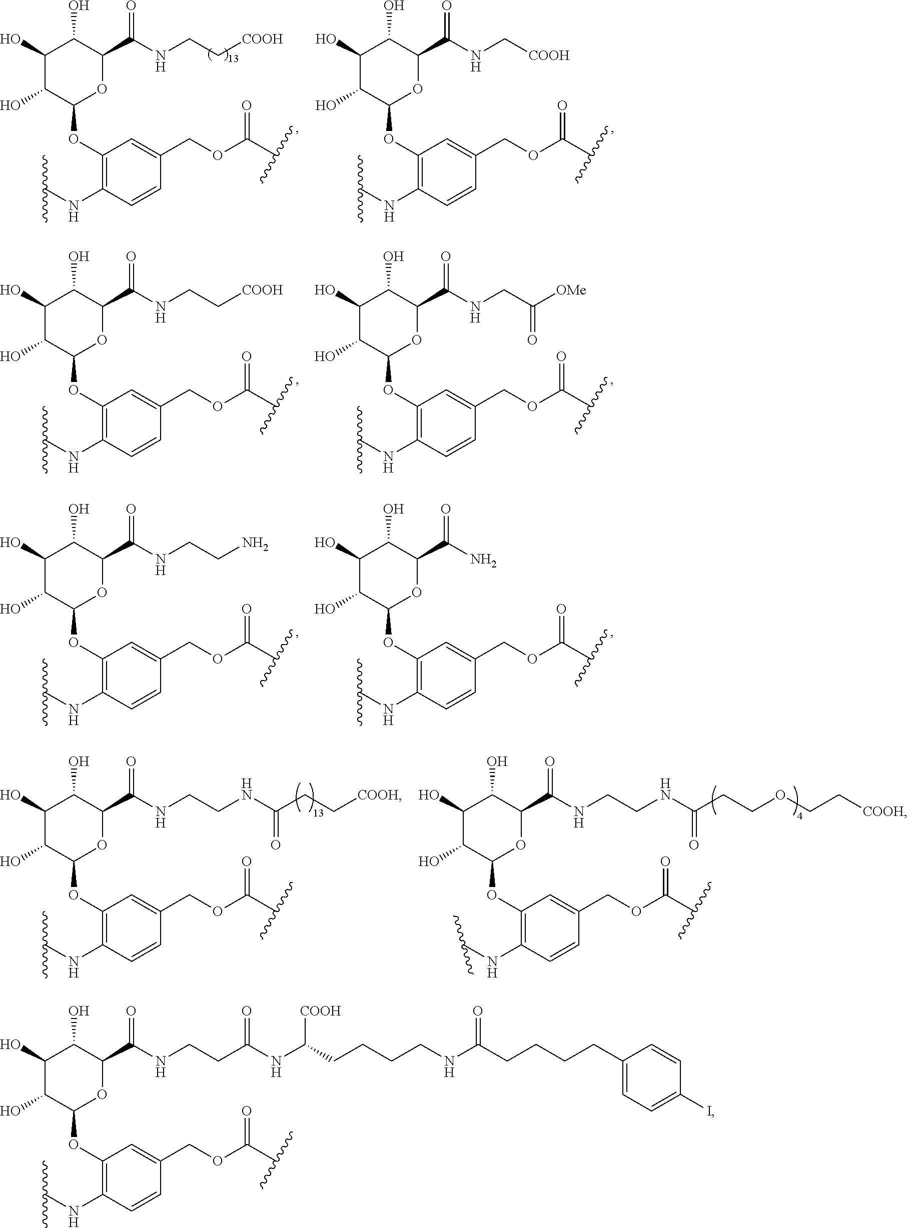

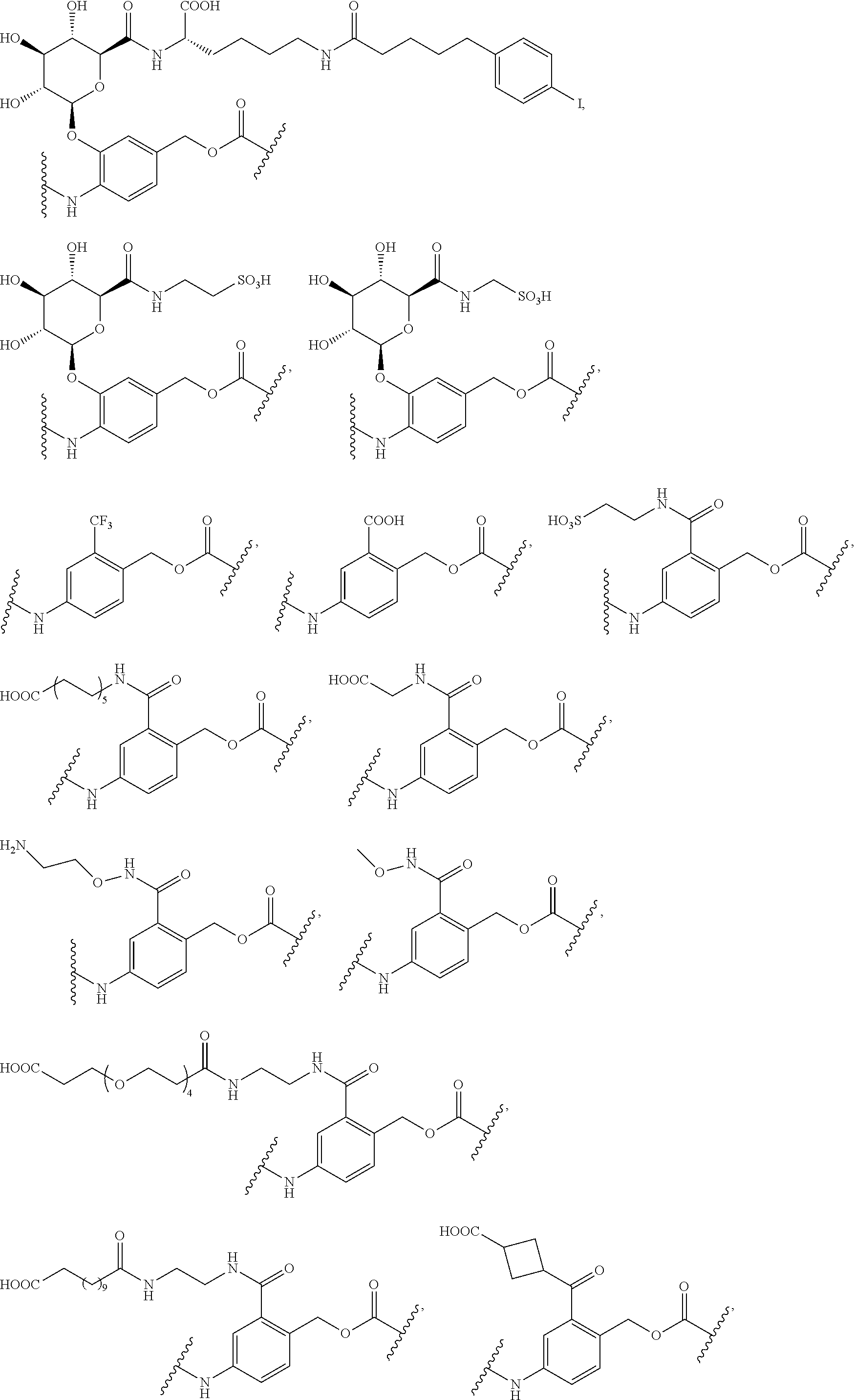

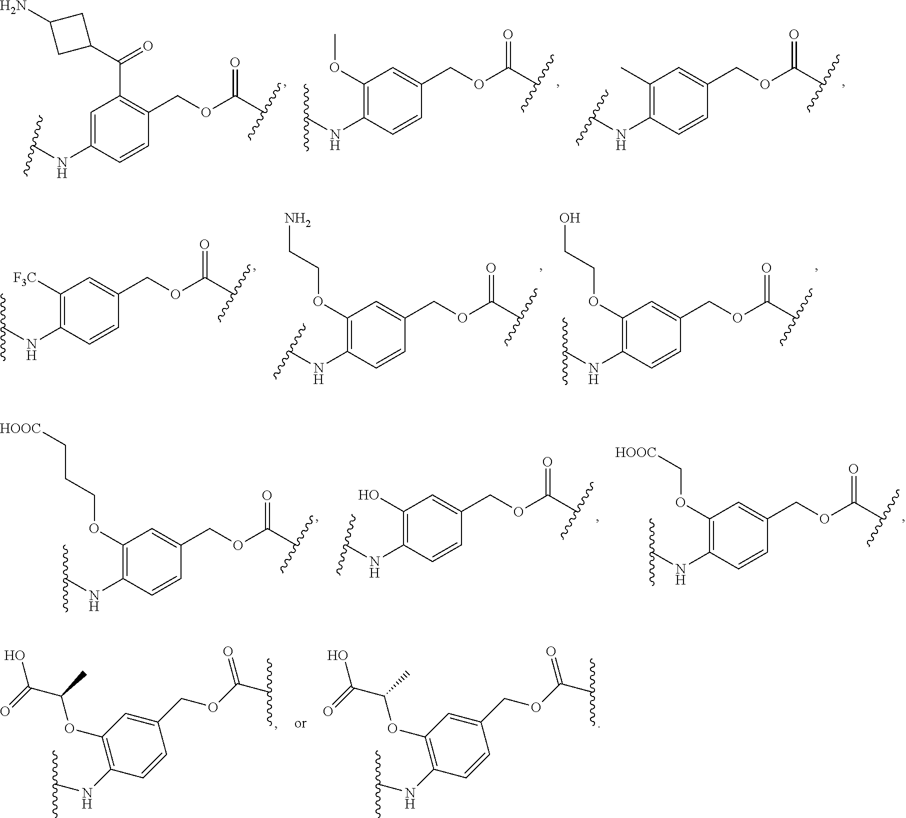

In some embodiments, the linker,

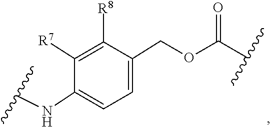

### of Formula (I) and (II) is:

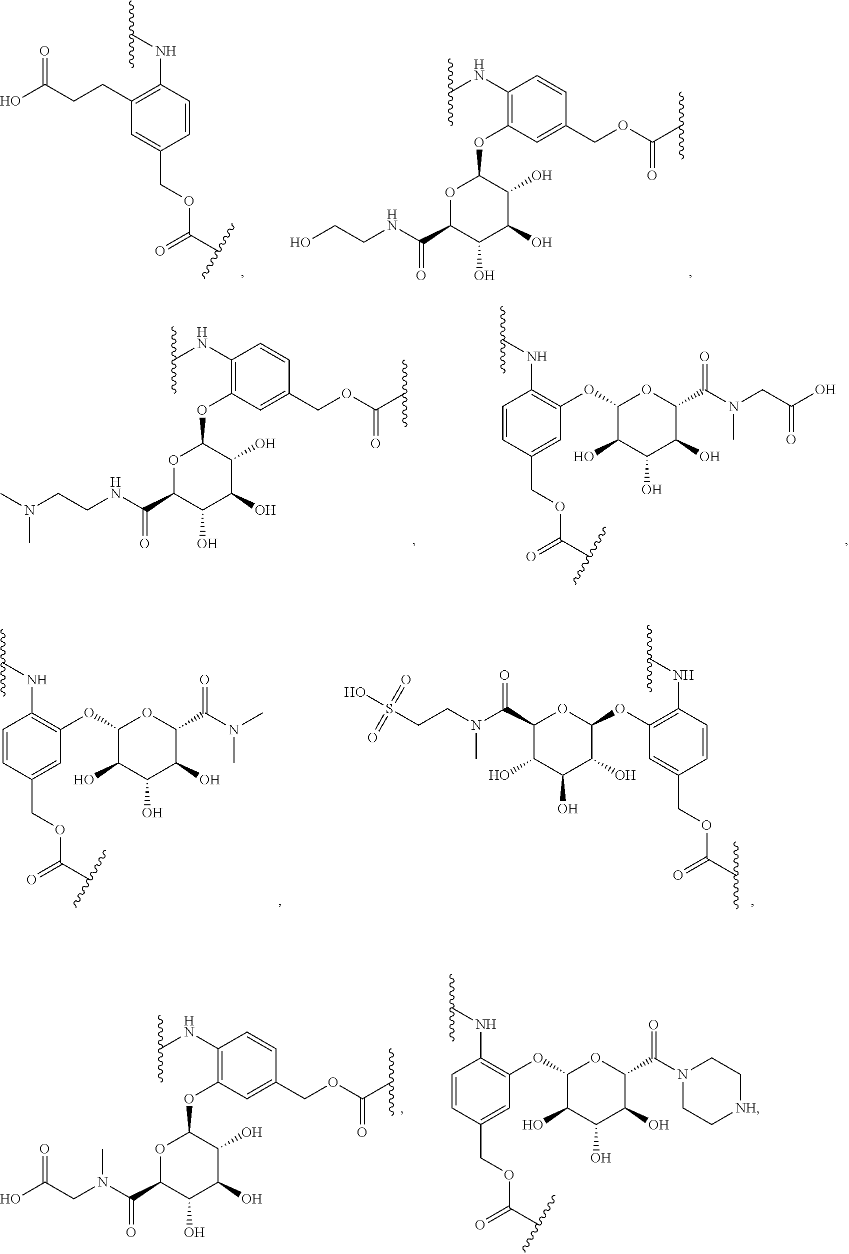

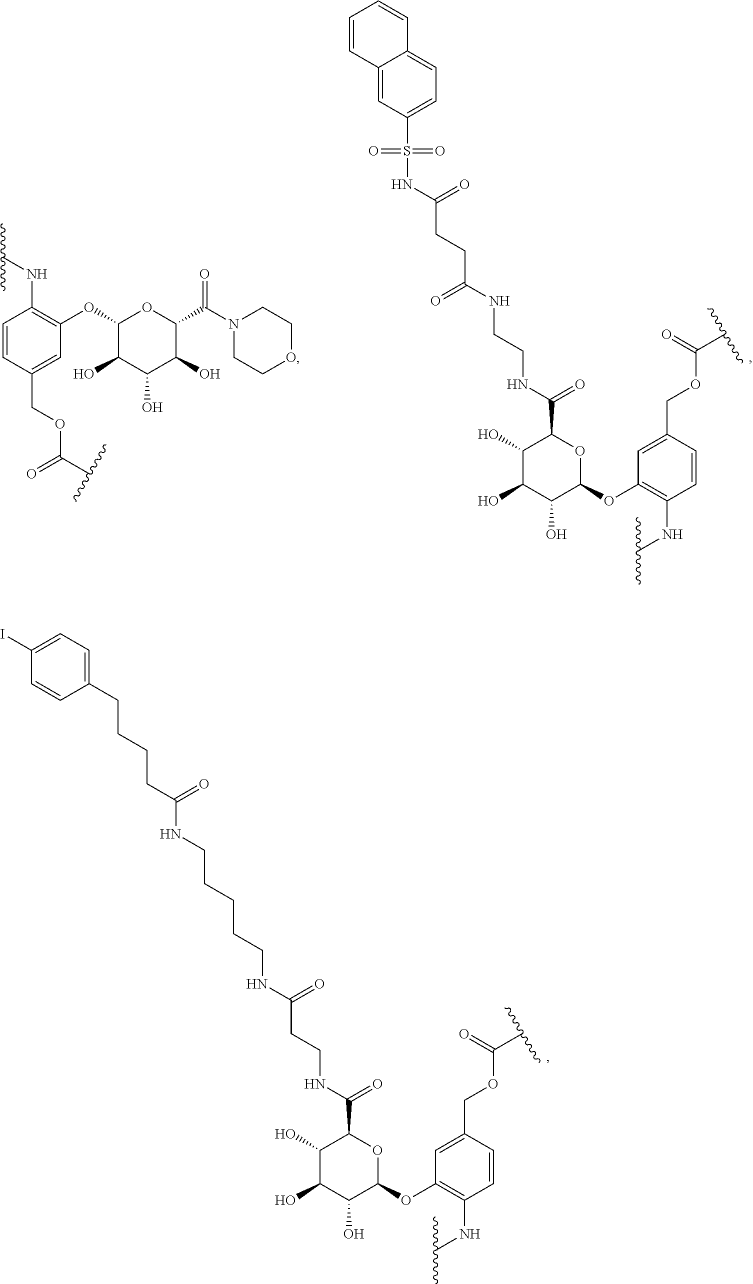

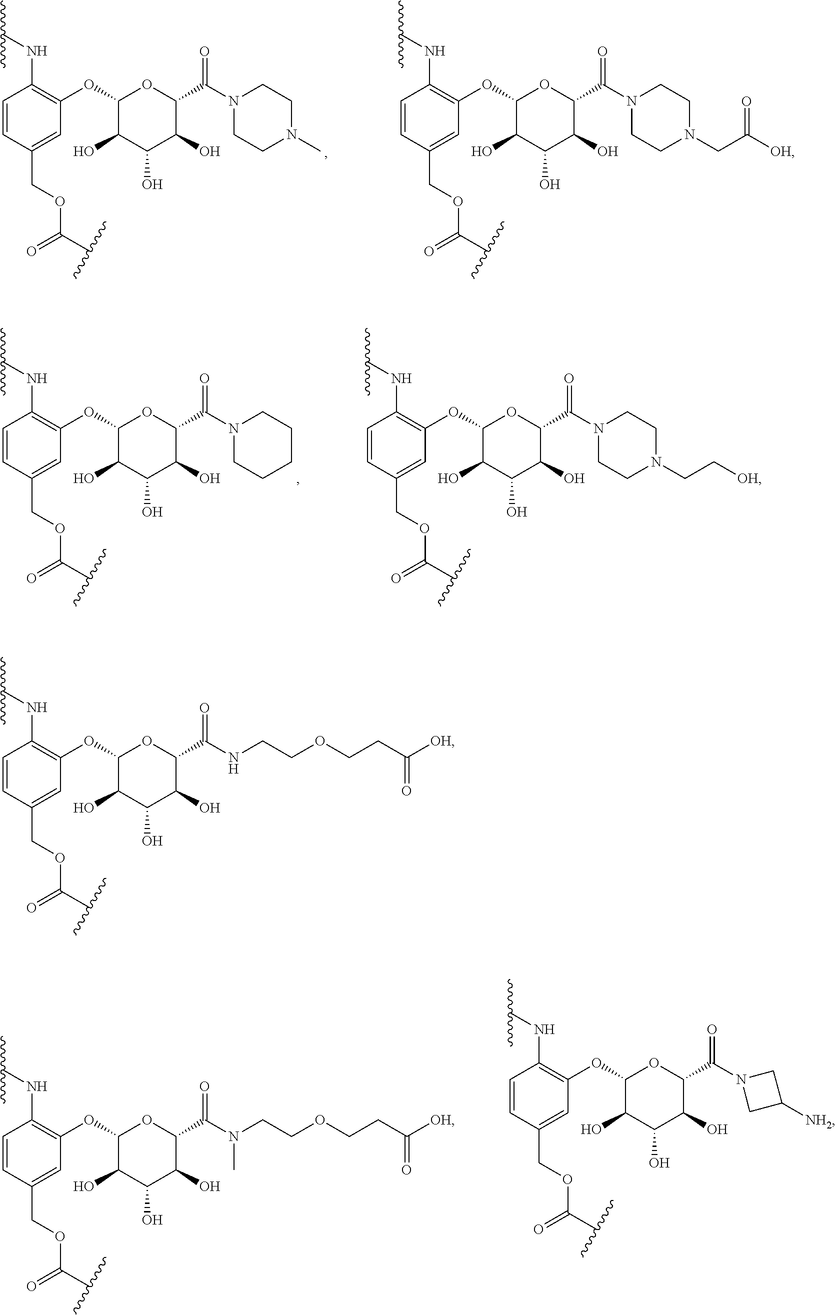

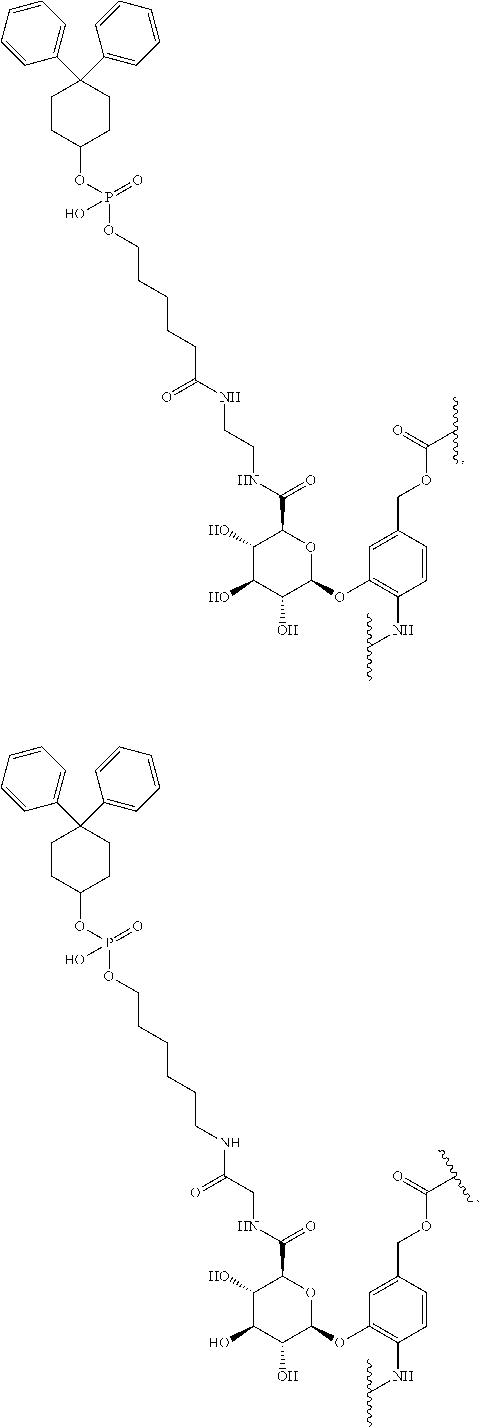

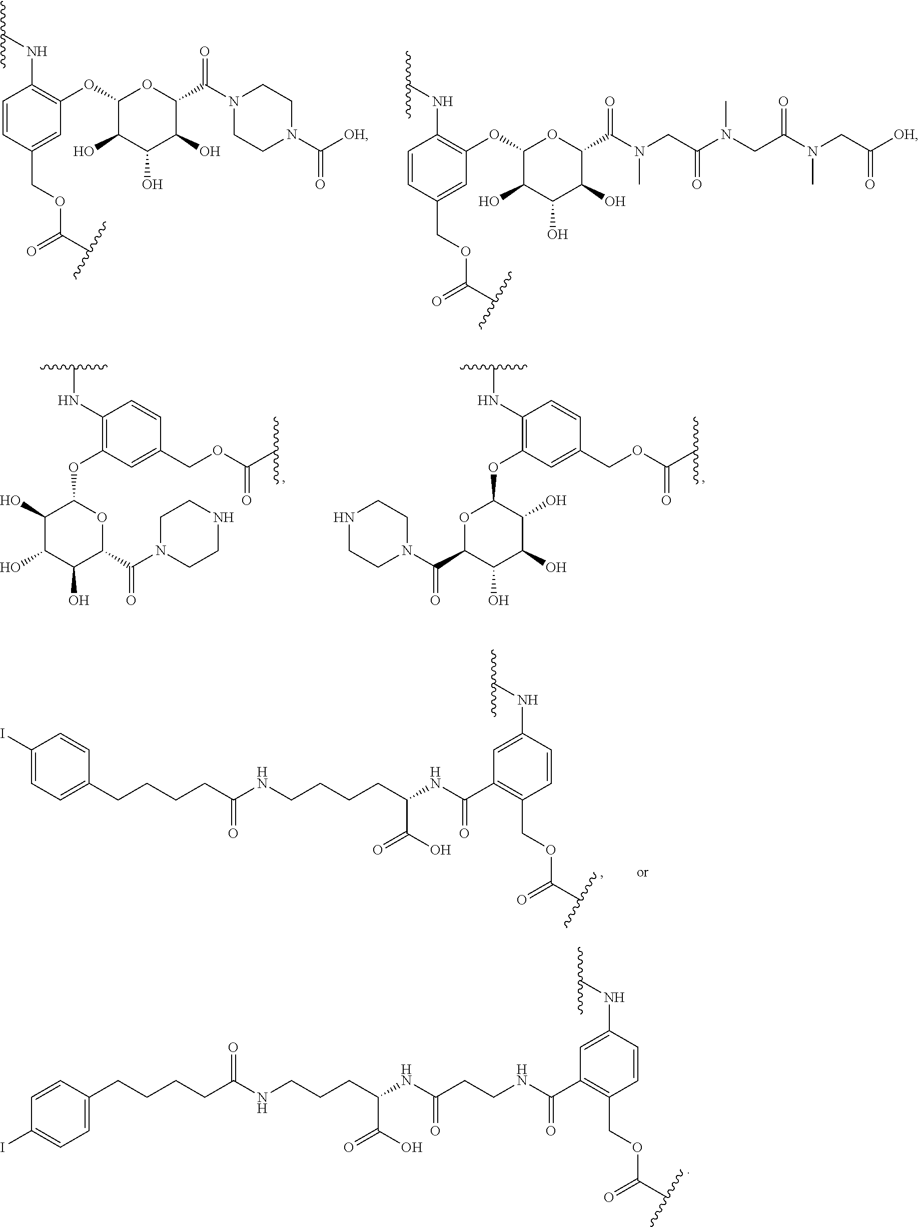

In some embodiments, the linker,

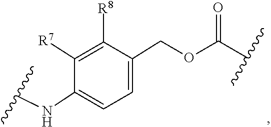

### of Formula (I) and (II) is:

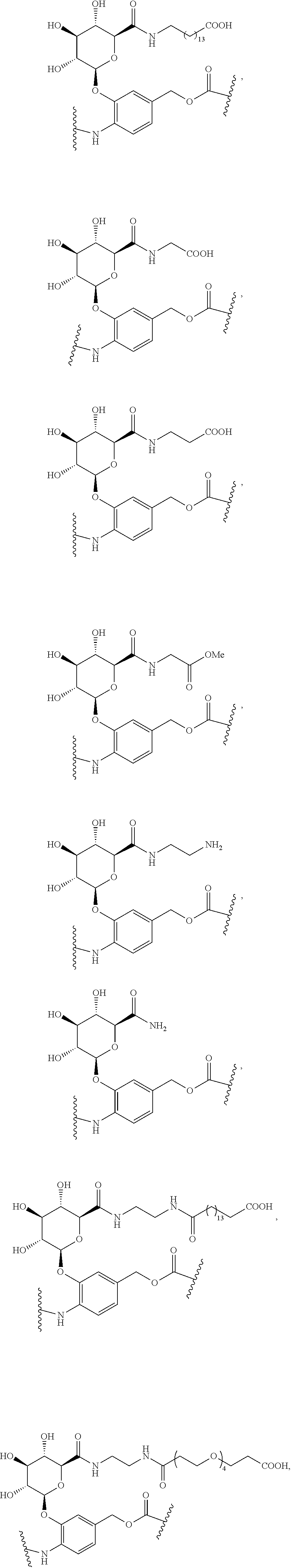

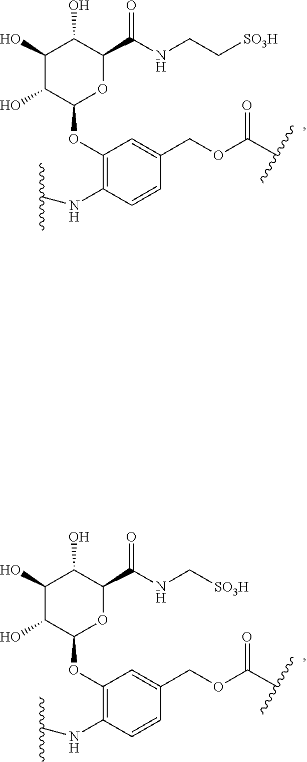 [SMILES: *Nc1ccc(COC(*)=O)cc1O[C@@H]1O[C@H](C(=O)NCCS(=O)(=O)O)[C@@H](O)[C@H](O)[C@H]1O ; *Nc1ccc(COC(*)=O)cc1O[C@@H]1O[C@H](C(=O)NCS(=O)(=O)O)[C@@H](O)[C@H](O)[C@H]1O]

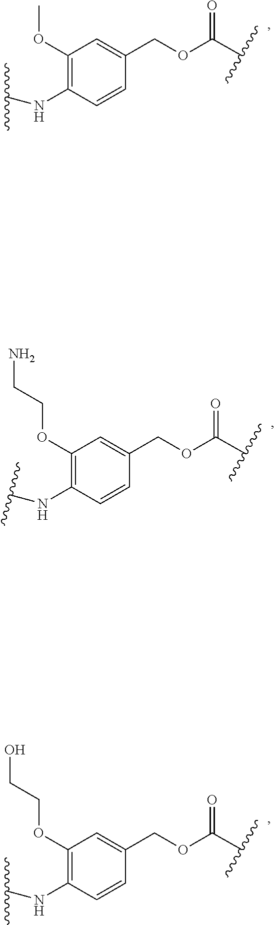 [SMILES: *Nc1ccc(COC(*)=O)cc1OC ; *Nc1ccc(COC(*)=O)cc1OCCN ; *Nc1ccc(COC(*)=O)cc1OCCO]

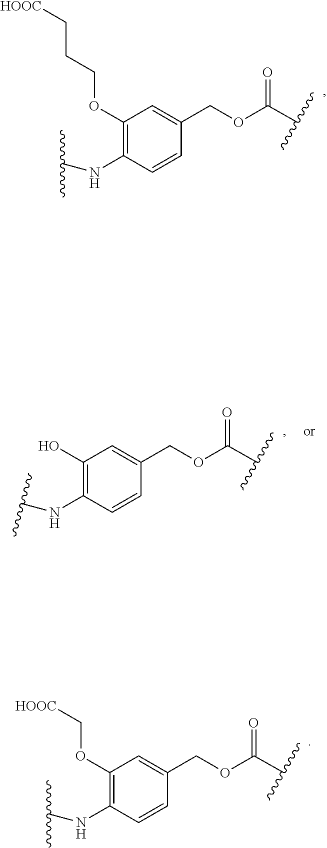 [SMILES: *Nc1ccc(COC(*)=O)cc1OCCCC(=O)O ; *Nc1ccc(COC(*)=O)cc1O ; *Nc1ccc(COC(*)=O)cc1OCC(=O)O]

In some embodiments, the linker,

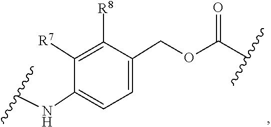

### of Formula (I) and (II) is:

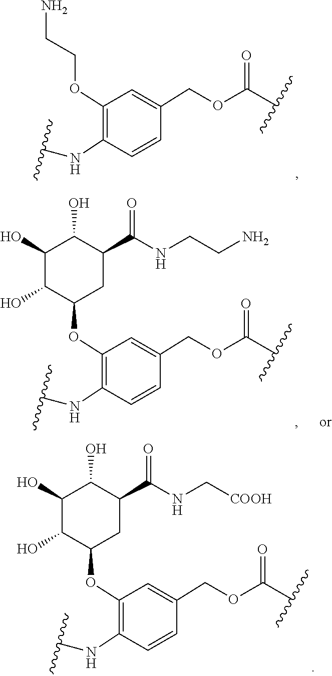

In some embodiments, in compounds of Formula (I) or Formula (II), each A is independently selected from natural or unnatural amino acids, wherein any free amine of an amino acid is optionally independently substituted with CH₃, wherein the natural or unnatural amino acids are selected from alanine (Ala), Ala(SO₃H), 3-(1-piperidinyl)alanine, cyclohexylalanine, arginine (Arg), asparagine (Asn), aspartate (Asp), cysteine (Cys), glutamine (Gln), glutamatic acid (Glu), glycine (Gly), leucine (Leu), lysine(Lys), methionine (Met), phenylalanine (Phe), homophenylalanine, proline (Pro), serine (Ser), 3-homoserine, tyrosine (Tyr), Tyr(SO₃H), tryptophan (Trp), valine (Val), citrulline, β-alanine, β3-homoserine, β3-homolysine, and β3-homoglutamic acid.

In some embodiments, each A is independently selected from natural or unnatural amino acids, wherein any free amine of an amino acid is optionally independently substituted with CH₃, wherein the natural or unnatural amino acids are selected from alanine (Ala), Ala(OH), Ala(SO₃H), glutamine (Gln), glutamatic acid (Glu), glycine (Gly), leucine (Leu), serine (Ser), tryptophan (Trp), valine (Val), and citrulline.

In some embodiments, w is 1, or 2, or 3, or 4, or 5, or 6. In some embodiments, w is 1. In some embodiments, w is 2. In some embodiments, w is 3. In some embodiments, w is 4. In some embodiments, w is 5. In some embodiments, w is 6. In some embodiments w is an integer selected from 2 or 3.

In some embodiments, (A)_{w} is valine-citrulline, valine-alanine, alanine(SO₃H)-valine-alanine, glutamic acid-valine-citrulline, glycine-valine-citrulline, glutamine-valine-citrulline, alanine(SO₃H)-valine-citrulline, serine-valine-citrulline, or tryptophane-valine-citrulline. In some embodiments, (A)_{w} is valine-citrulline, valine-alanine, or glutamic acid-valine-citrulline.

In some embodiments, the linker,

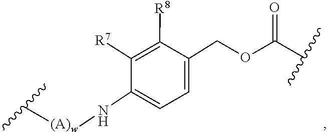

### of Formula (I) or Formula (II) is

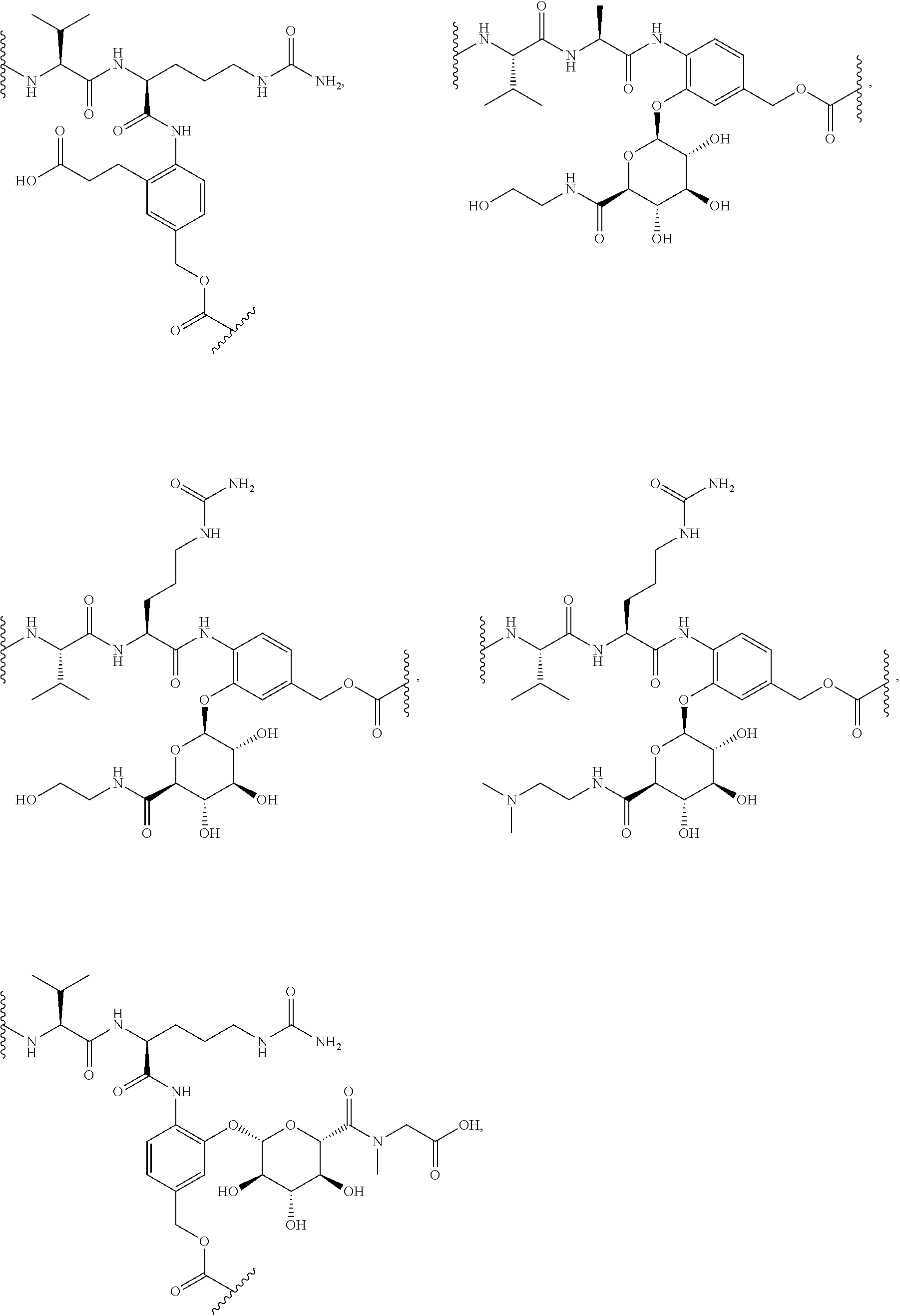

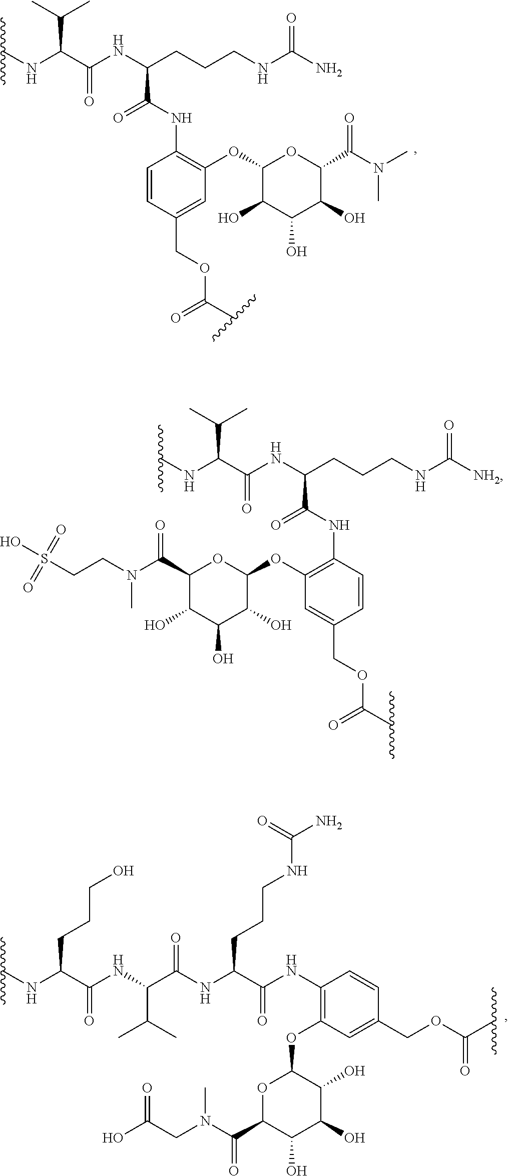 [SMILES: *N[C@H](C(=O)N[C@@H](CCCNC(N)=O)C(=O)Nc1ccc(COC(*)=O)cc1O[C@@H]1O[C@H](C(=O)N(C)C)[C@@H](O)[C@H](O)[C@H]1O)C(C)C ; *N[C@H](C(=O)N[C@@H](CCCNC(N)=O)C(=O)Nc1ccc(COC(*)=O)cc1O[C@@H]1O[C@H](C(=O)N(C)CCS(=O)(=O)O)[C@@H](O)[C@H](O)[C@H]1O)C(C)C ; *N[C@@H](CCCO)C(=O)N[C@H](C(=O)N[C@@H](CCCNC(N)=O)C(=O)Nc1ccc(COC(*)=O)cc1O[C@@H]1O[C@H](C(=O)N(C)CC(=O)O)[C@@H](O)[C@H](O)[C@H]1O)C(C)C]

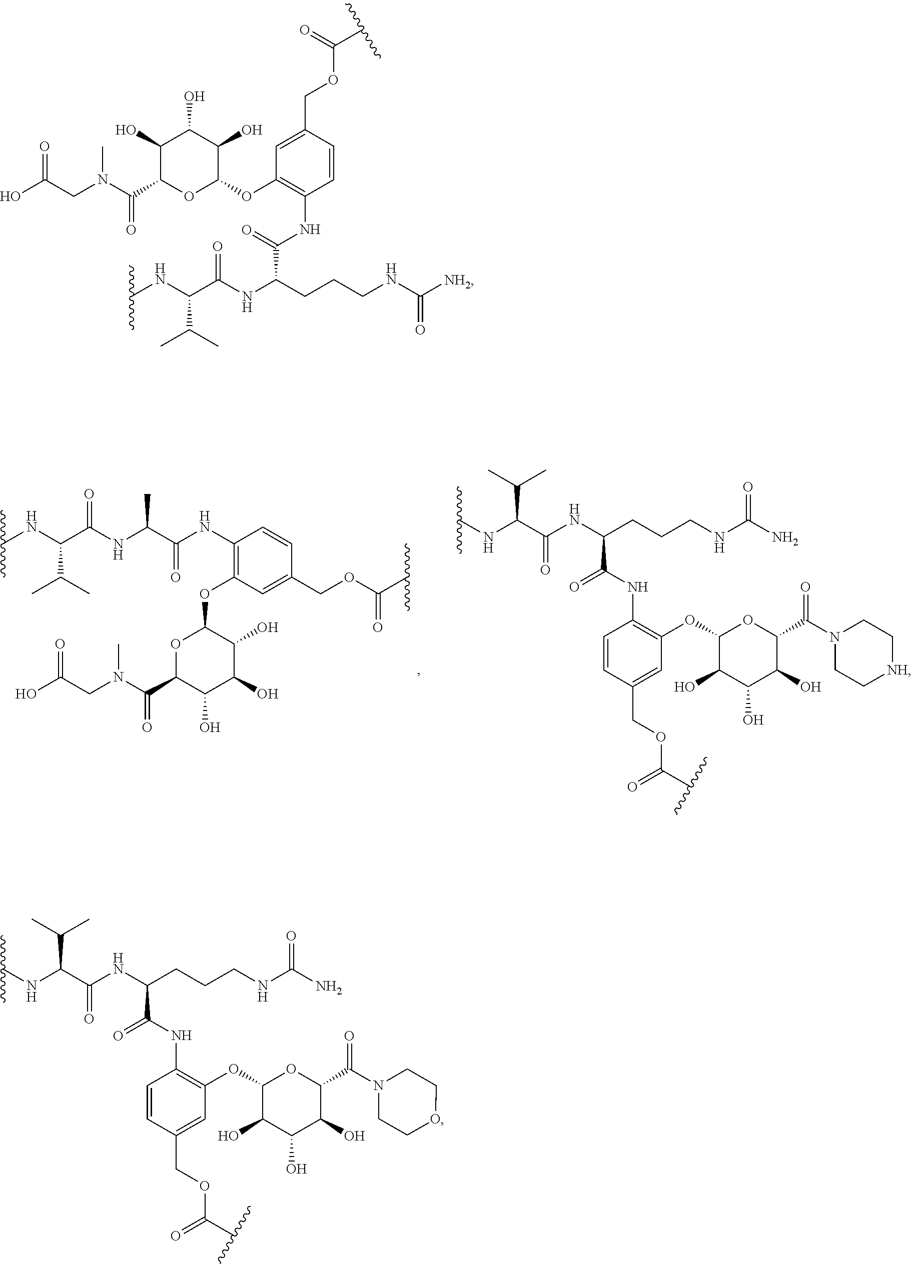 [SMILES: *N[C@H](C(=O)N[C@@H](CCCNC(N)=O)C(=O)Nc1ccc(COC(*)=O)cc1O[C@@H]1O[C@H](C(=O)N(C)CC(=O)O)[C@@H](O)[C@H](O)[C@H]1O)C(C)C ; *N[C@H](C(=O)N[C@@H](C)C(=O)Nc1ccc(COC(*)=O)cc1O[C@@H]1O[C@H](C(=O)N(C)CC(=O)O)[C@@H](O)[C@H](O)[C@H]1O)C(C)C ; *N[C@H](C(=O)N[C@@H](CCCNC(N)=O)C(=O)Nc1ccc(COC(*)=O)cc1O[C@@H]1O[C@H](C(=O)N2CCNCC2)[C@@H](O)[C@H](O)[C@H]1O)C(C)C ; *N[C@H](C(=O)N[C@@H](CCCNC(N)=O)C(=O)Nc1ccc(COC(*)=O)cc1O[C@@H]1O[C@H](C(=O)N2CCOCC2)[C@@H](O)[C@H](O)[C@H]1O)C(C)C]

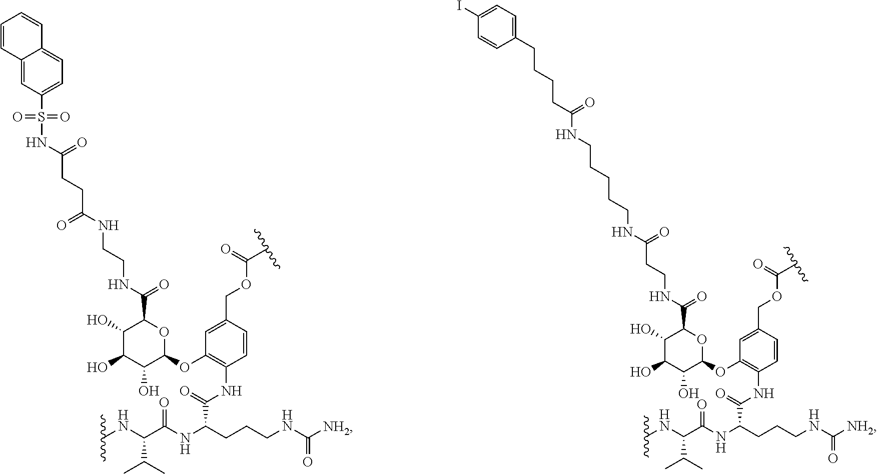 [SMILES: *N[C@H](C(=O)N[C@@H](CCCNC(N)=O)C(=O)Nc1ccc(COC(*)=O)cc1O[C@@H]1O[C@H](C(=O)NCCNC(=O)CCC(=O)NS(=O)(=O)c2ccc3ccccc3c2)[C@@H](O)[C@H](O)[C@H]1O)C(C)C ; *N[C@H](C(=O)N[C@@H](CCCNC(N)=O)C(=O)Nc1ccc(COC(*)=O)cc1O[C@@H]1O[C@H](C(=O)NCCC(=O)NCCCCCNC(=O)CCCCc2ccc(I)cc2)[C@@H](O)[C@H](O)[C@H]1O)C(C)C]

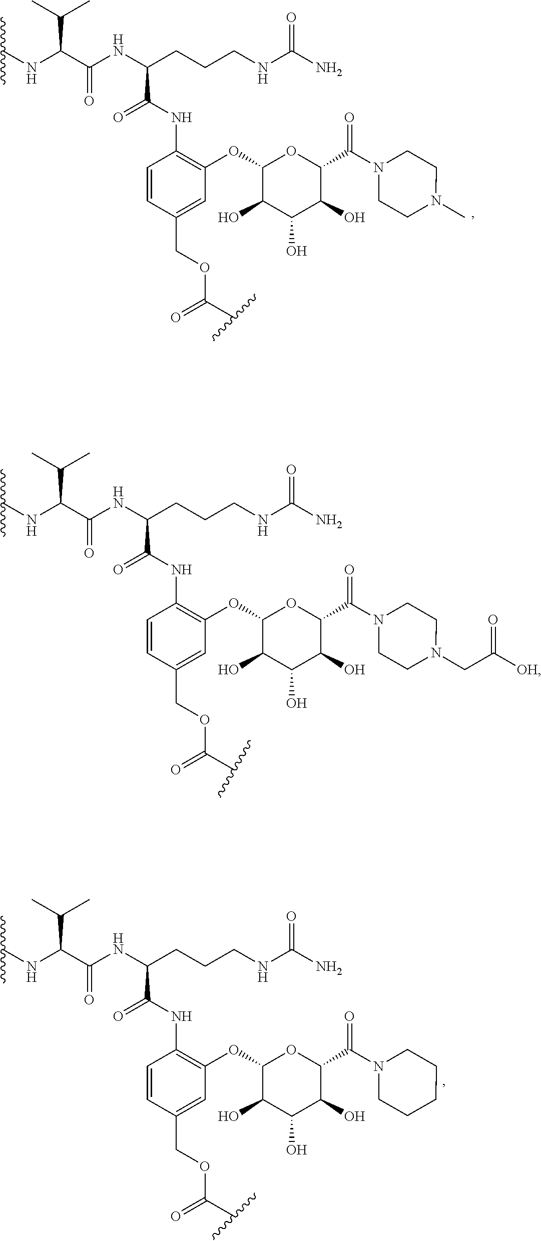 [SMILES: *N[C@H](C(=O)N[C@@H](CCCNC(N)=O)C(=O)Nc1ccc(COC(*)=O)cc1O[C@@H]1O[C@H](C(=O)N2CCN(C)CC2)[C@@H](O)[C@H](O)[C@H]1O)C(C)C ; *N[C@H](C(=O)N[C@@H](CCCNC(N)=O)C(=O)Nc1ccc(COC(*)=O)cc1O[C@@H]1O[C@H](C(=O)N2CCN(CC(=O)O)CC2)[C@@H](O)[C@H](O)[C@H]1O)C(C)C ; *N[C@H](C(=O)N[C@@H](CCCNC(N)=O)C(=O)Nc1ccc(COC(*)=O)cc1O[C@@H]1O[C@H](C(=O)N2CCCCC2)[C@@H](O)[C@H](O)[C@H]1O)C(C)C]

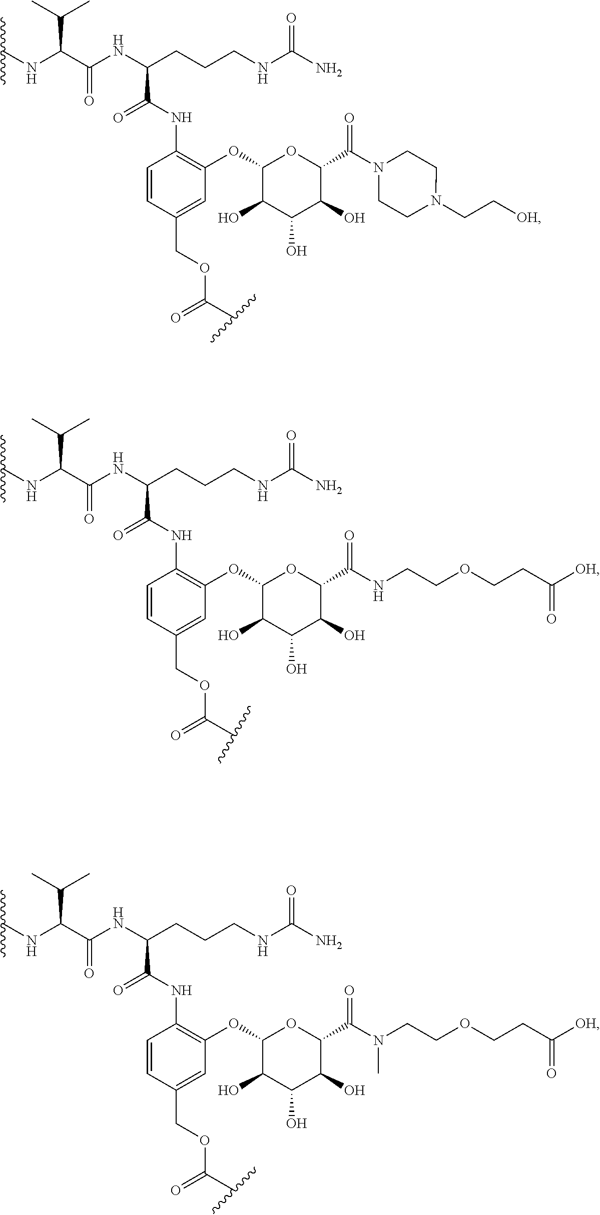 [SMILES: *N[C@H](C(=O)N[C@@H](CCCNC(N)=O)C(=O)Nc1ccc(COC(*)=O)cc1O[C@@H]1O[C@H](C(=O)N2CCN(CCO)CC2)[C@@H](O)[C@H](O)[C@H]1O)C(C)C ; *N[C@H](C(=O)N[C@@H](CCCNC(N)=O)C(=O)Nc1ccc(COC(*)=O)cc1O[C@@H]1O[C@H](C(=O)NCCOCCC(=O)O)[C@@H](O)[C@H](O)[C@H]1O)C(C)C ; *N[C@H](C(=O)N[C@@H](CCCNC(N)=O)C(=O)Nc1ccc(COC(*)=O)cc1O[C@@H]1O[C@H](C(=O)N(C)CCOCCC(=O)O)[C@@H](O)[C@H](O)[C@H]1O)C(C)C]

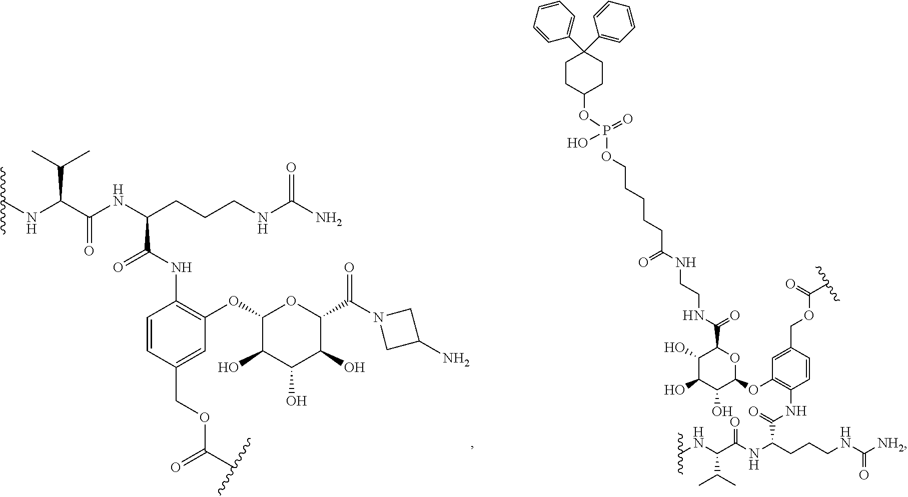 [SMILES: *N[C@H](C(=O)N[C@@H](CCCNC(N)=O)C(=O)Nc1ccc(COC(*)=O)cc1O[C@@H]1O[C@H](C(=O)N2CC(N)C2)[C@@H](O)[C@H](O)[C@H]1O)C(C)C ; *N[C@H](C(=O)N[C@@H](CCCNC(N)=O)C(=O)Nc1ccc(COC(*)=O)cc1O[C@@H]1O[C@H](C(=O)NCCNC(=O)CCCCCOP(=O)(O)OC2CCC(c3ccccc3)(c3ccccc3)CC2)[C@@H](O)[C@H](O)[C@H]1O)C(C)C]

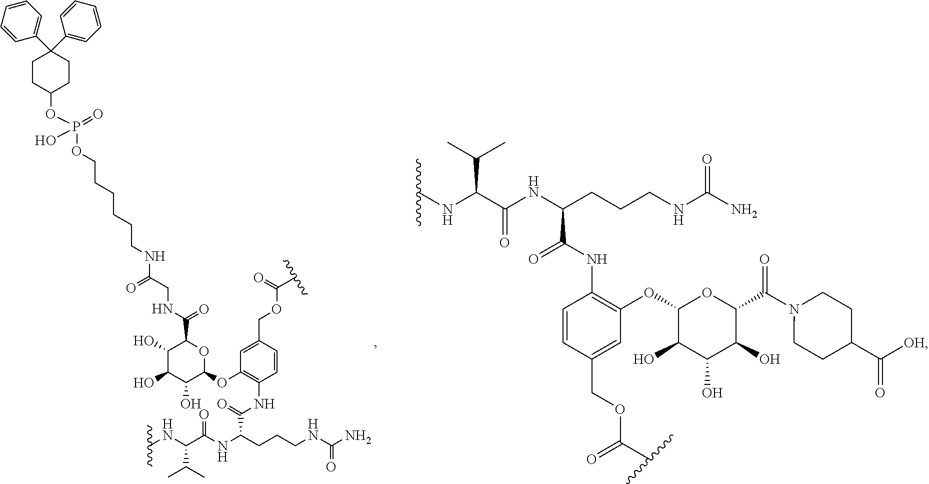 [SMILES: *N[C@H](C(=O)N[C@@H](CCCNC(N)=O)C(=O)Nc1ccc(COC(*)=O)cc1O[C@@H]1O[C@H](C(=O)NCC(=O)NCCCCCCOP(=O)(O)OC2CCC(c3ccccc3)(c3ccccc3)CC2)[C@@H](O)[C@H](O)[C@H]1O)C(C)C ; *N[C@H](C(=O)N[C@@H](CCCNC(N)=O)C(=O)Nc1ccc(COC(*)=O)cc1O[C@@H]1O[C@H](C(=O)N2CCC(C(=O)O)CC2)[C@@H](O)[C@H](O)[C@H]1O)C(C)C]

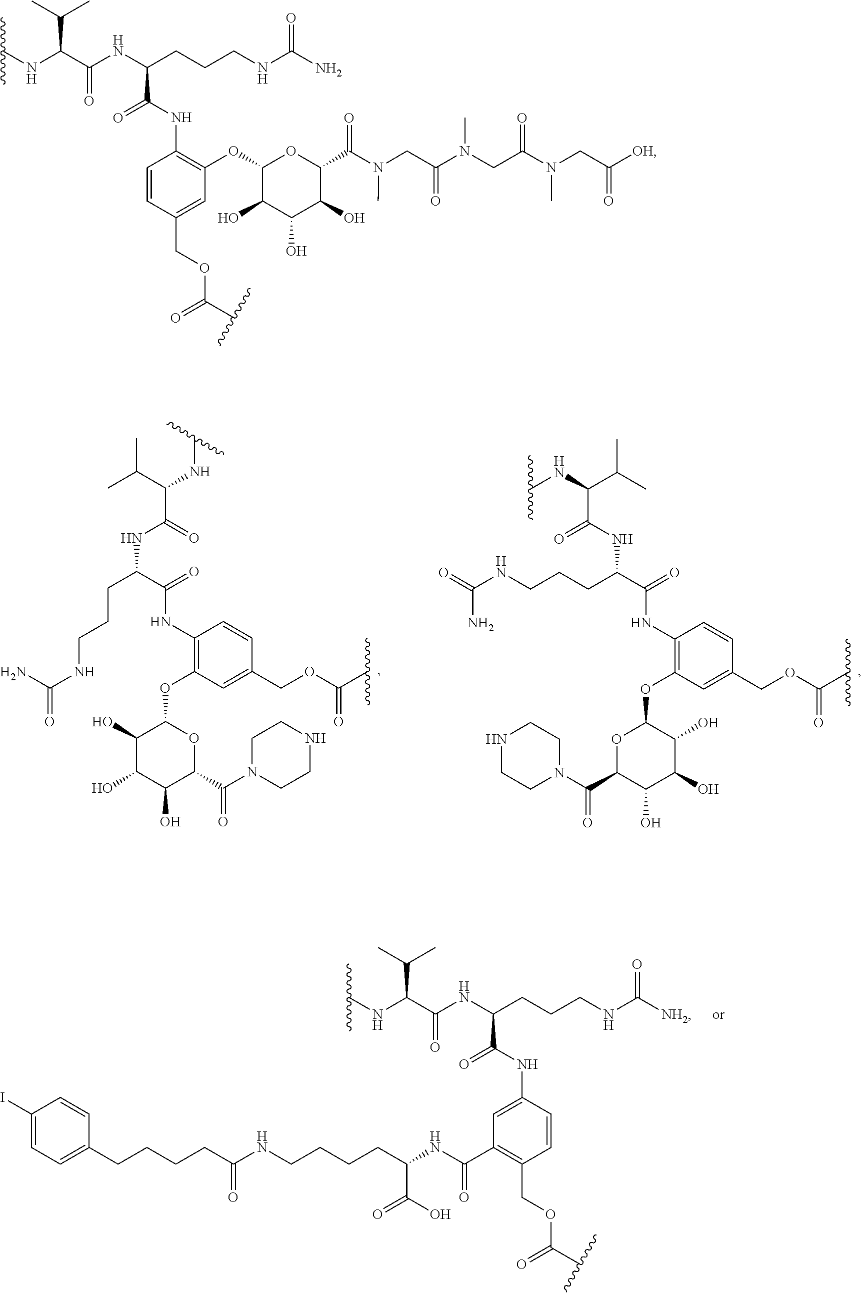 [SMILES: *N[C@H](C(=O)N[C@@H](CCCNC(N)=O)C(=O)Nc1ccc(COC(*)=O)cc1O[C@@H]1O[C@H](C(=O)N(C)CC(=O)N(C)CC(=O)N(C)CC(=O)O)[C@@H](O)[C@H](O)[C@H]1O)C(C)C ; *N[C@H](C(=O)N[C@@H](CCCNC(N)=O)C(=O)Nc1ccc(COC(*)=O)cc1O[C@@H]1O[C@H](C(=O)N2CCNCC2)[C@@H](O)[C@H](O)[C@H]1O)C(C)C ; *N[C@H](C(=O)N[C@@H](CCCNC(N)=O)C(=O)Nc1ccc(COC(*)=O)cc1O[C@@H]1O[C@H](C(=O)N2CCNCC2)[C@@H](O)[C@H](O)[C@H]1O)C(C)C ; *N[C@H](C(=O)N[C@@H](CCCNC(N)=O)C(=O)Nc1ccc(COC(*)=O)c(C(=O)N[C@@H](CCCCNC(=O)CCCCc2ccc(I)cc2)C(=O)O)c1)C(C)C]

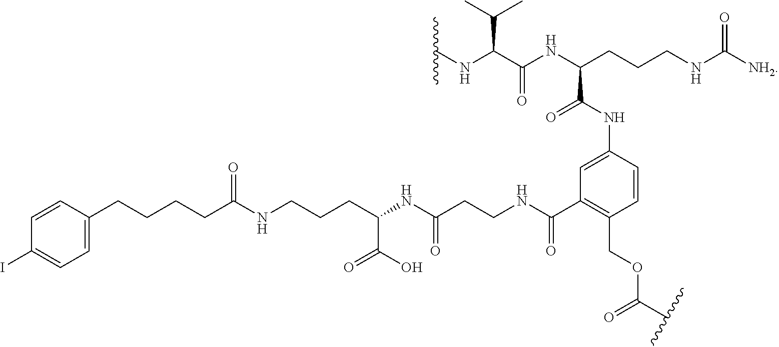 [SMILES: *N[C@H](C(=O)N[C@@H](CCCNC(N)=O)C(=O)Nc1ccc(COC(*)=O)c(C(=O)NCCC(=O)N[C@@H](CCCNC(=O)CCCCc2ccc(I)cc2)C(=O)O)c1)C(C)C]

In some embodiments, the linker,

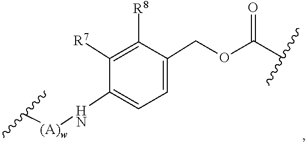

### of Formula (I) or Formula (II) is

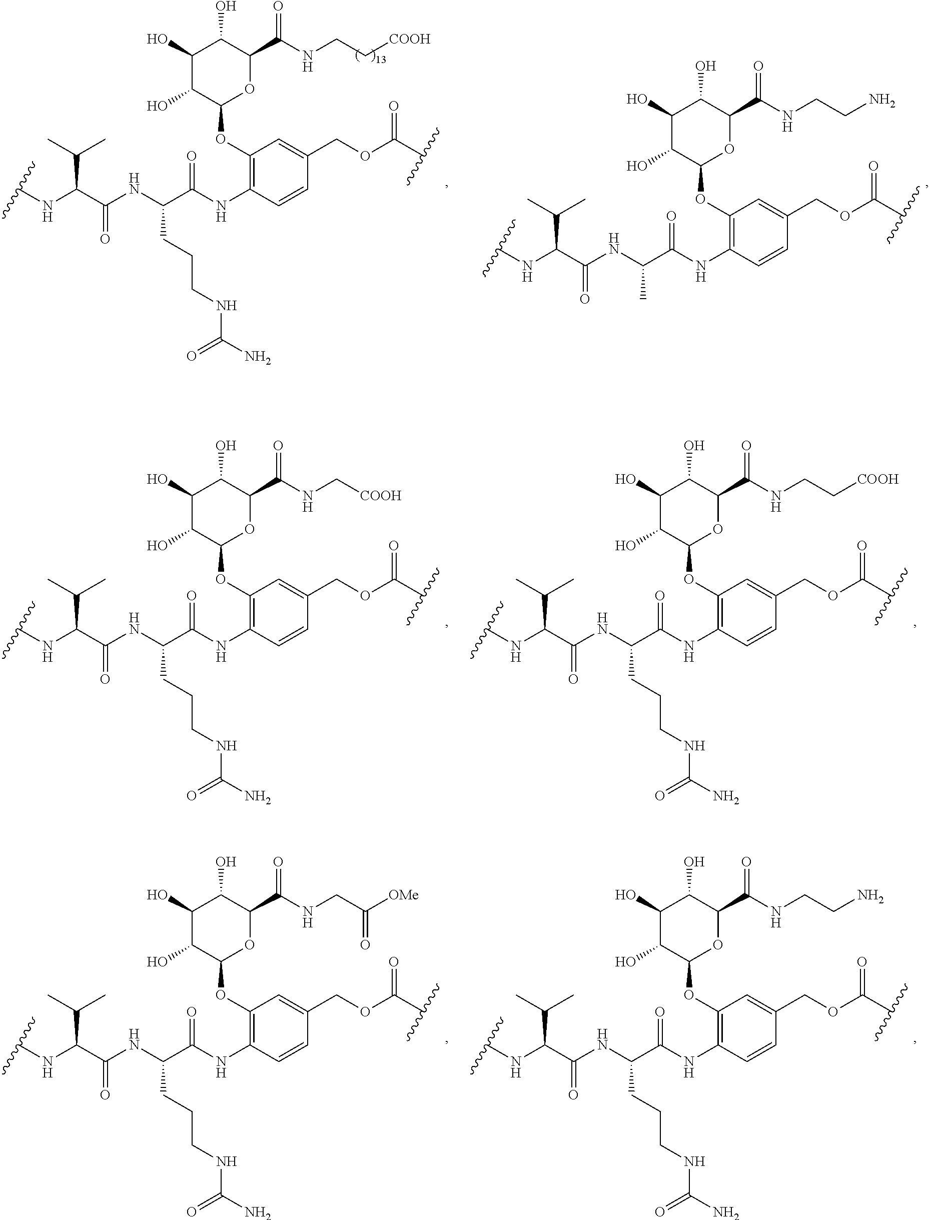

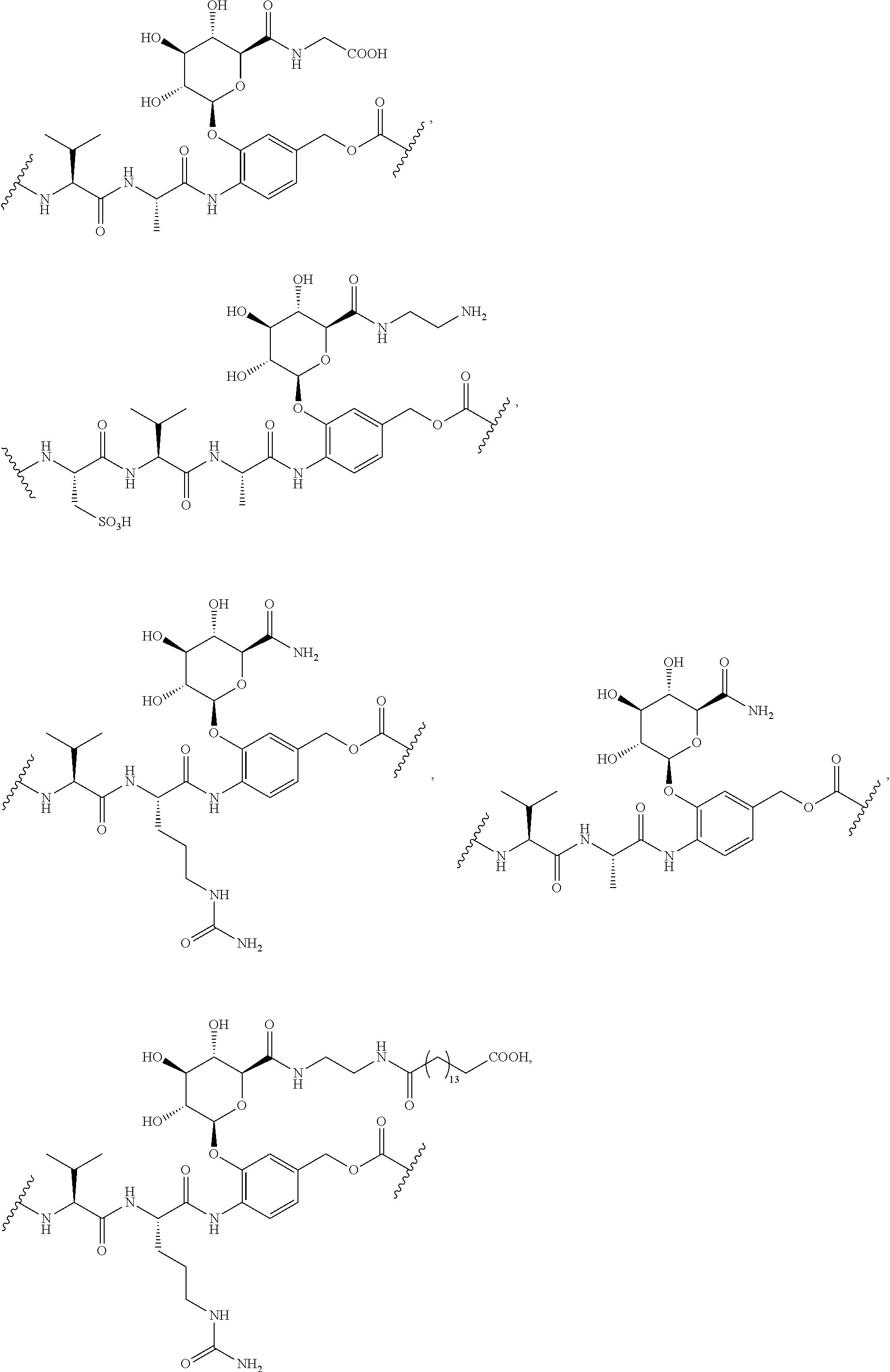

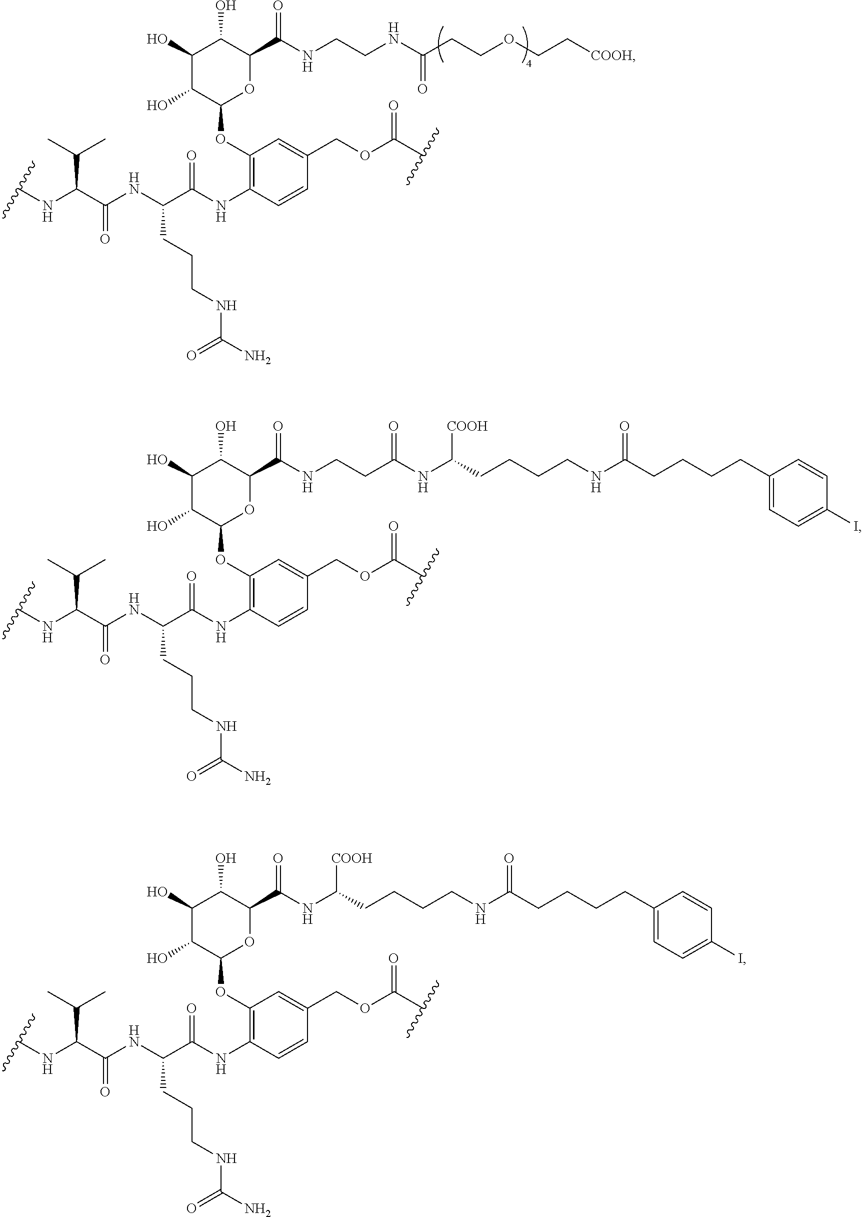

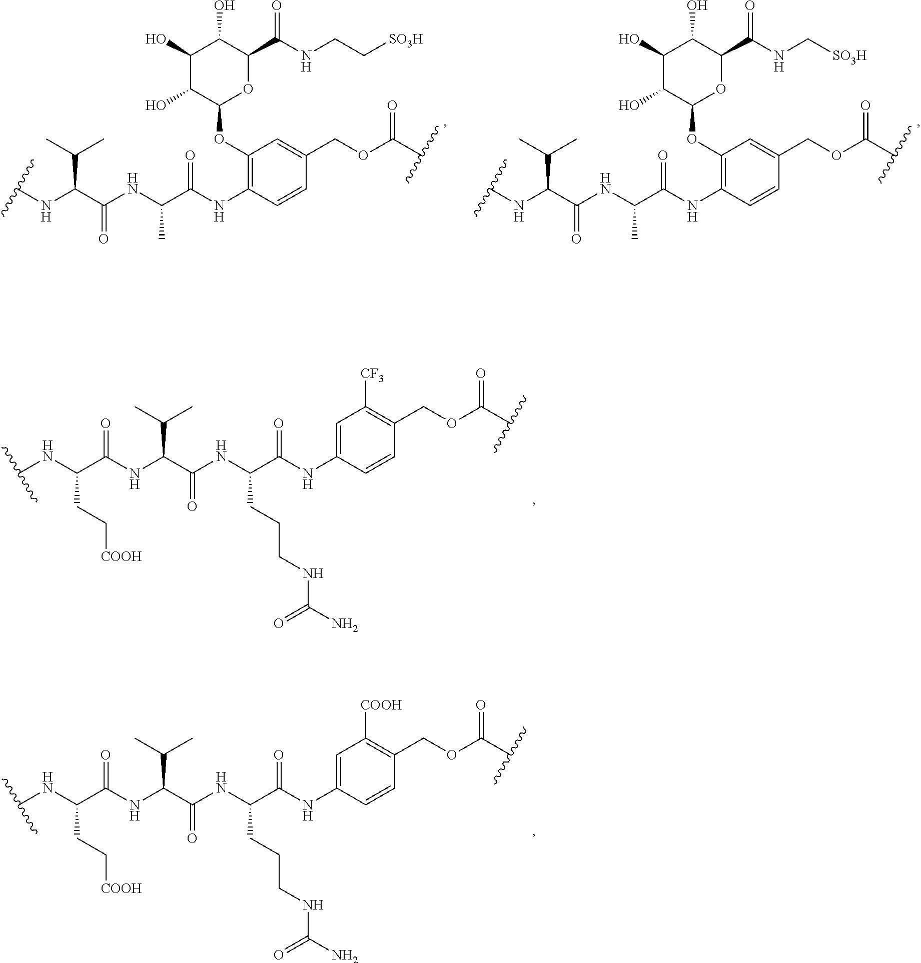 [SMILES: *N[C@H](C(=O)N[C@@H](C)C(=O)Nc1ccc(COC(*)=O)cc1O[C@@H]1O[C@H](C(=O)NCCS(=O)(=O)O)[C@@H](O)[C@H](O)[C@H]1O)C(C)C ; *N[C@H](C(=O)N[C@@H](C)C(=O)Nc1ccc(COC(*)=O)cc1O[C@@H]1O[C@H](C(=O)NCS(=O)(=O)O)[C@@H](O)[C@H](O)[C@H]1O)C(C)C ; *N[C@@H](CCC(=O)O)C(=O)N[C@H](C(=O)N[C@@H](CCCNC(N)=O)C(=O)Nc1ccc(COC(*)=O)c(C(F)(F)F)c1)C(C)C ; *N[C@@H](CCC(=O)O)C(=O)N[C@H](C(=O)N[C@@H](CCCNC(N)=O)C(=O)Nc1ccc(COC(*)=O)c(C(=O)O)c1)C(C)C]

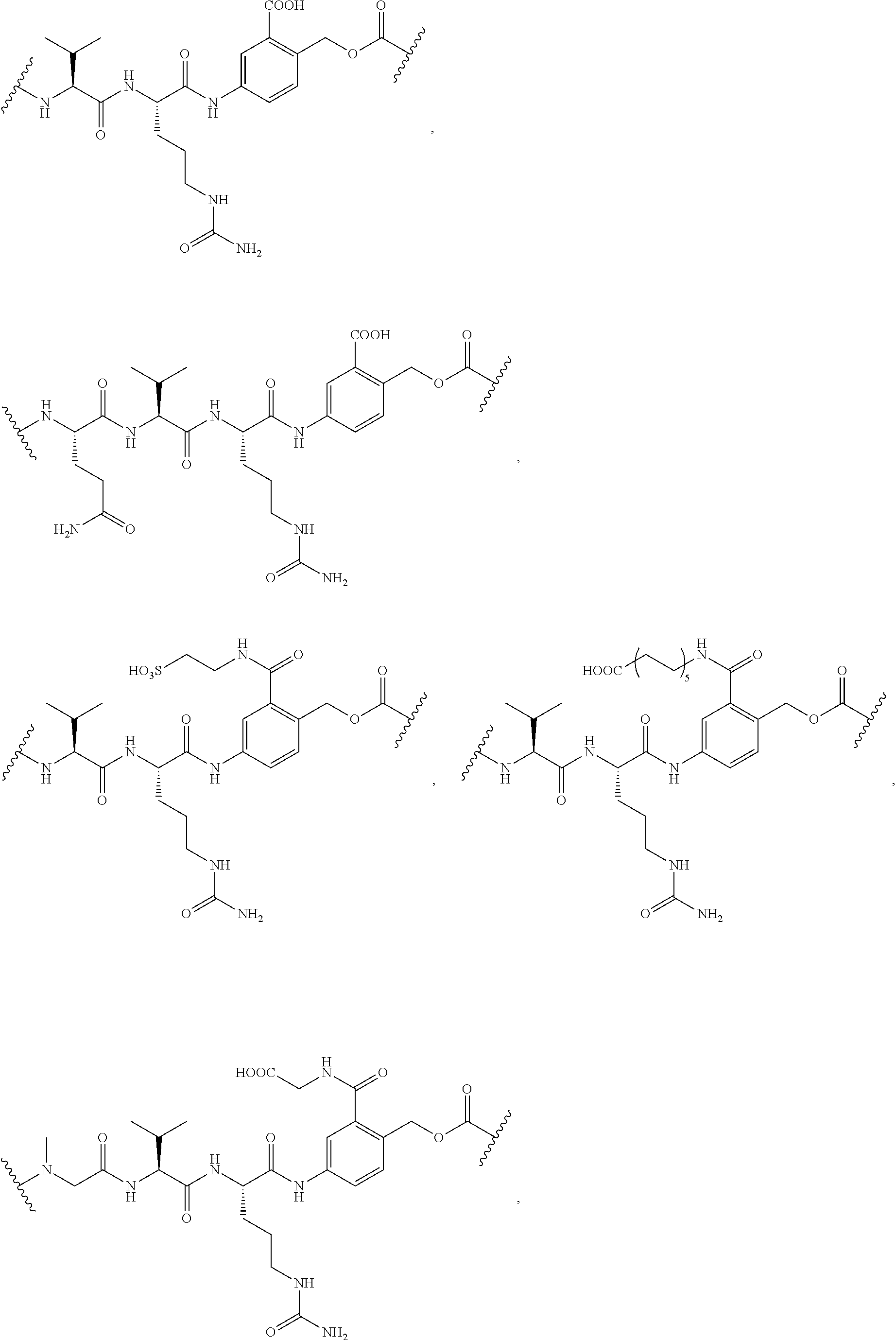

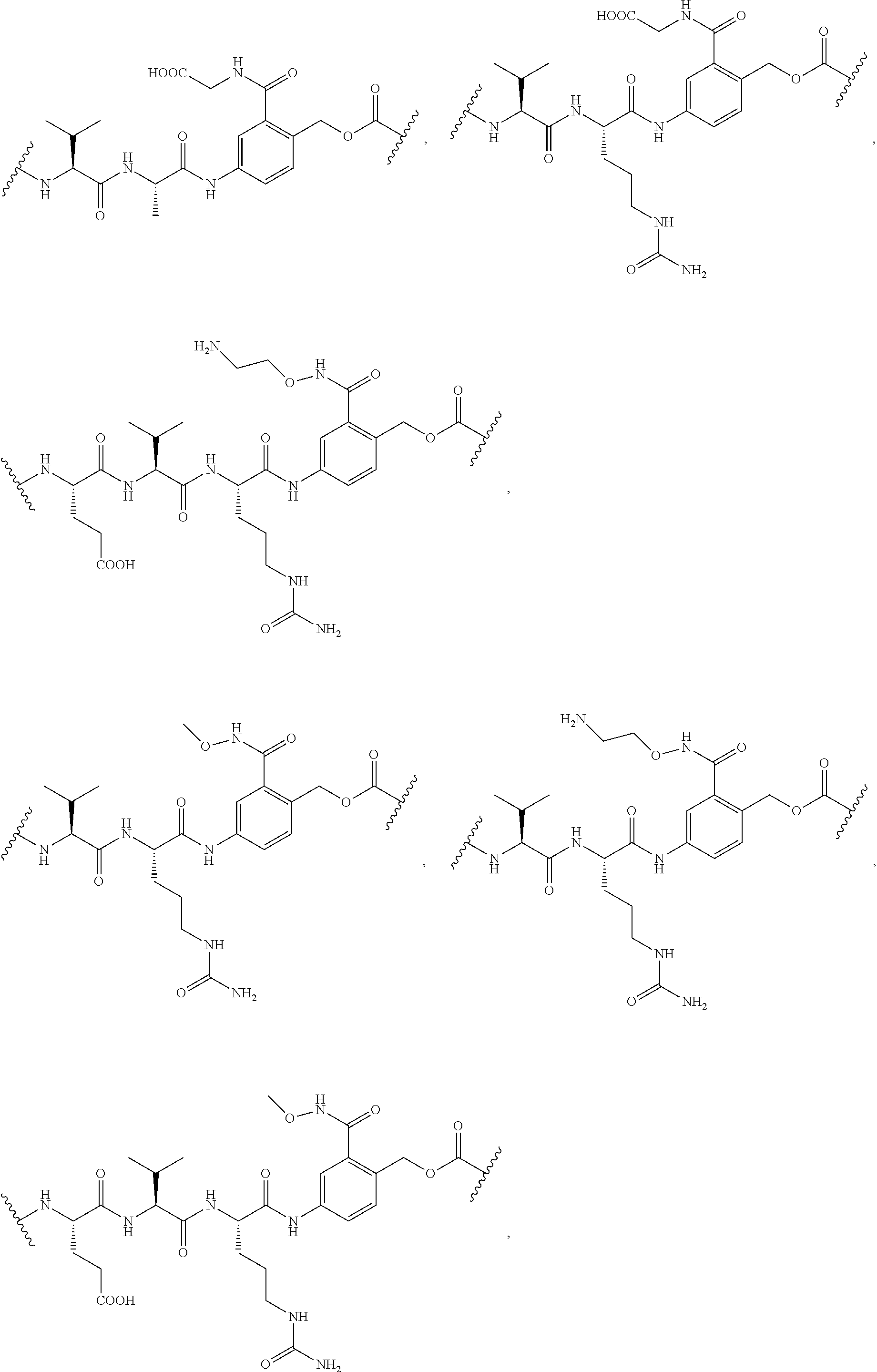 [SMILES: *N[C@H](C(=O)N[C@@H](C)C(=O)Nc1ccc(COC(*)=O)c(C(=O)NCC(=O)O)c1)C(C)C ; *N[C@H](C(=O)N[C@@H](CCCNC(N)=O)C(=O)Nc1ccc(COC(*)=O)c(C(=O)NCC(=O)O)c1)C(C)C ; *N[C@@H](CCC(=O)O)C(=O)N[C@H](C(=O)N[C@@H](CCCNC(N)=O)C(=O)Nc1ccc(COC(*)=O)c(C(=O)NOCCN)c1)C(C)C ; *N[C@H](C(=O)N[C@@H](CCCNC(N)=O)C(=O)Nc1ccc(COC(*)=O)c(C(=O)NOC)c1)C(C)C ; *N[C@H](C(=O)N[C@@H](CCCNC(N)=O)C(=O)Nc1ccc(COC(*)=O)c(C(=O)NOCCN)c1)C(C)C ; *N[C@@H](CCC(=O)O)C(=O)N[C@H](C(=O)N[C@@H](CCCNC(N)=O)C(=O)Nc1ccc(COC(*)=O)c(C(=O)NOC)c1)C(C)C]

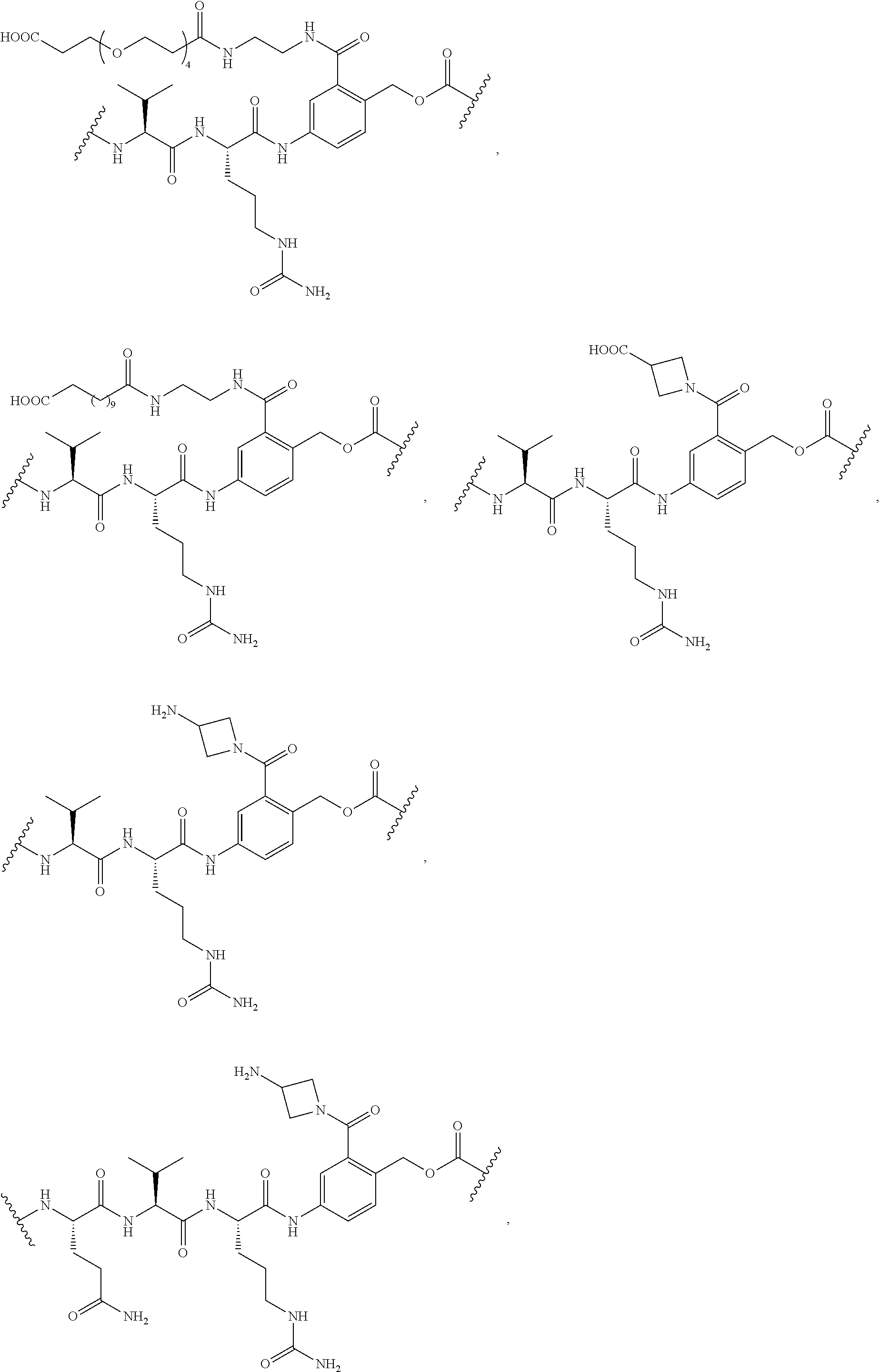

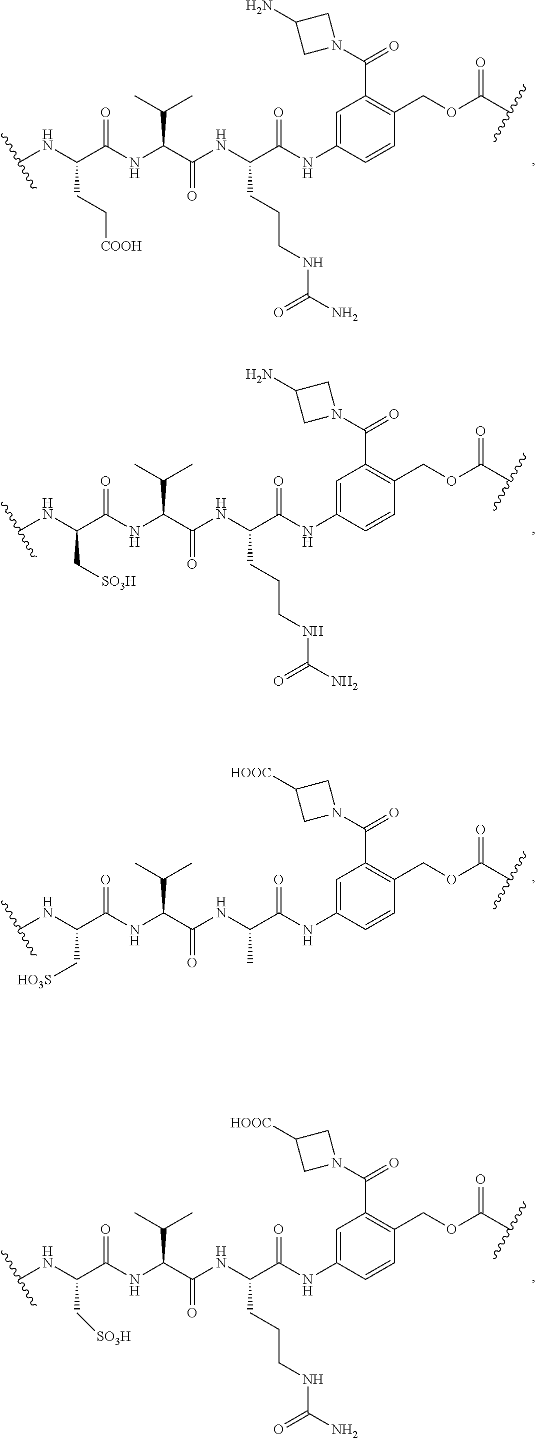 [SMILES: *N[C@@H](CCC(=O)O)C(=O)N[C@H](C(=O)N[C@@H](CCCNC(N)=O)C(=O)Nc1ccc(COC(*)=O)c(C(=O)N2CC(N)C2)c1)C(C)C ; *N[C@H](CS(=O)(=O)O)C(=O)N[C@H](C(=O)N[C@@H](CCCNC(N)=O)C(=O)Nc1ccc(COC(*)=O)c(C(=O)N2CC(N)C2)c1)C(C)C ; *N[C@@H](CS(=O)(=O)O)C(=O)N[C@H](C(=O)N[C@@H](C)C(=O)Nc1ccc(COC(*)=O)c(C(=O)N2CC(C(=O)O)C2)c1)C(C)C ; *N[C@@H](CS(=O)(=O)O)C(=O)N[C@H](C(=O)N[C@@H](CCCNC(N)=O)C(=O)Nc1ccc(COC(*)=O)c(C(=O)N2CC(C(=O)O)C2)c1)C(C)C]

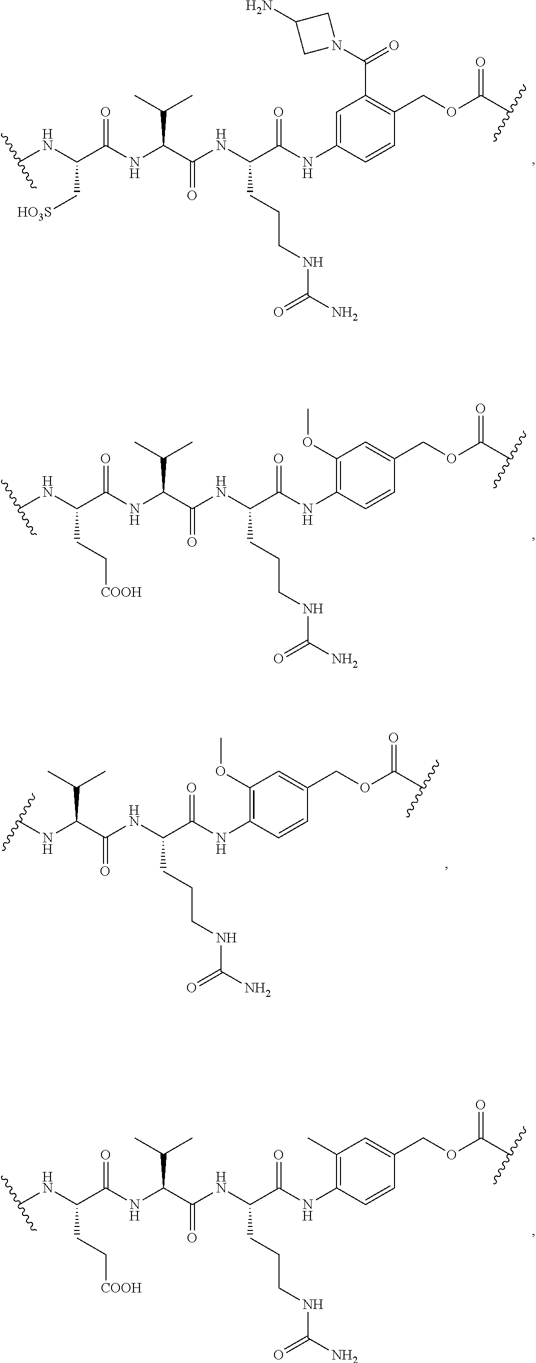 [SMILES: *N[C@@H](CS(=O)(=O)O)C(=O)N[C@H](C(=O)N[C@@H](CCCNC(N)=O)C(=O)Nc1ccc(COC(*)=O)c(C(=O)N2CC(N)C2)c1)C(C)C ; *N[C@@H](CCC(=O)O)C(=O)N[C@H](C(=O)N[C@@H](CCCNC(N)=O)C(=O)Nc1ccc(COC(*)=O)cc1OC)C(C)C ; *N[C@H](C(=O)N[C@@H](CCCNC(N)=O)C(=O)Nc1ccc(COC(*)=O)cc1OC)C(C)C ; *N[C@@H](CCC(=O)O)C(=O)N[C@H](C(=O)N[C@@H](CCCNC(N)=O)C(=O)Nc1ccc(COC(*)=O)cc1C)C(C)C]

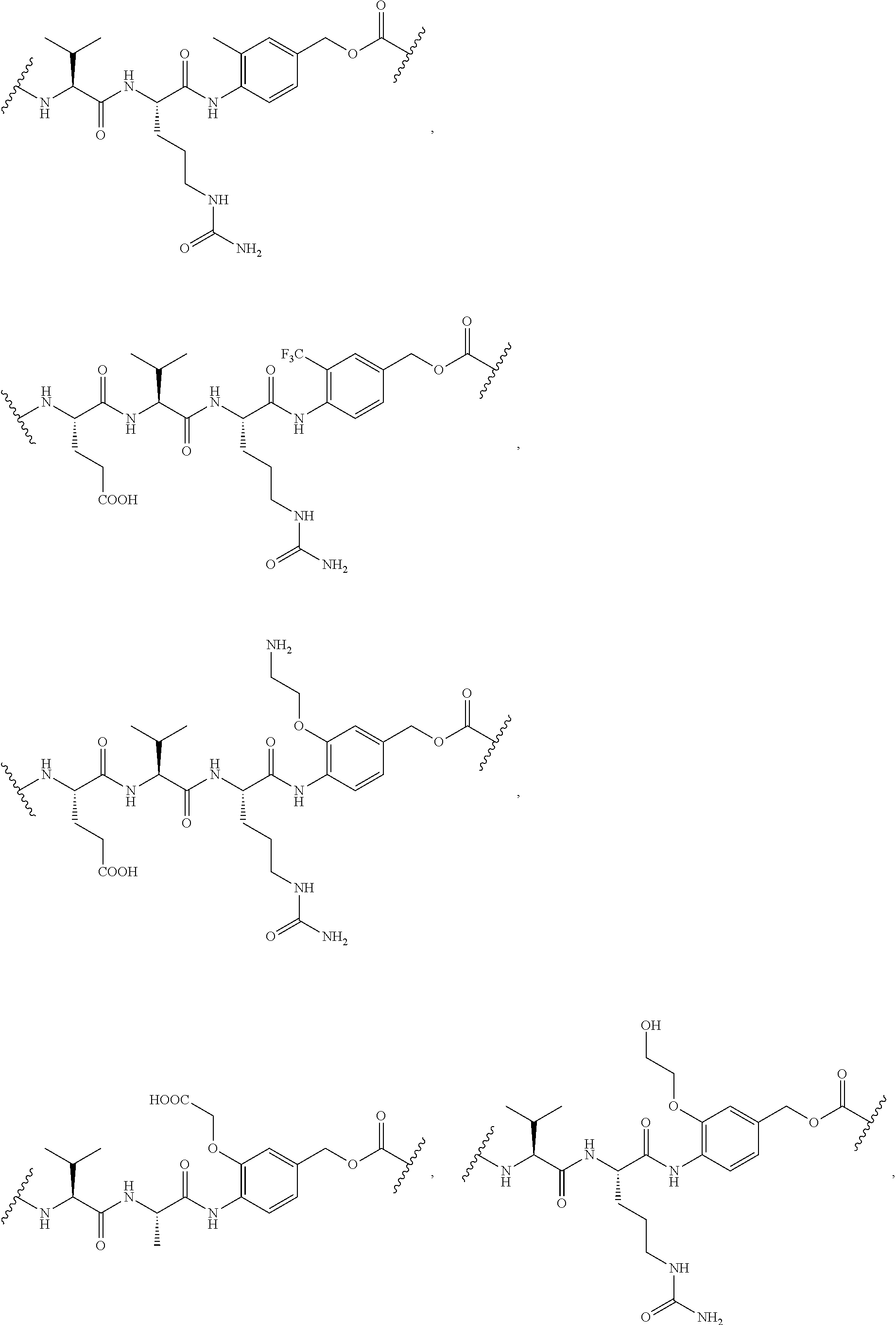 [SMILES: *N[C@H](C(=O)N[C@@H](CCCNC(N)=O)C(=O)Nc1ccc(COC(*)=O)cc1C)C(C)C ; *N[C@@H](CCC(=O)O)C(=O)N[C@H](C(=O)N[C@@H](CCCNC(N)=O)C(=O)Nc1ccc(COC(*)=O)cc1C(F)(F)F)C(C)C ; *N[C@@H](CCC(=O)O)C(=O)N[C@H](C(=O)N[C@@H](CCCNC(N)=O)C(=O)Nc1ccc(COC(*)=O)cc1OCCN)C(C)C ; *N[C@H](C(=O)N[C@@H](C)C(=O)Nc1ccc(COC(*)=O)cc1OCC(=O)O)C(C)C ; *N[C@H](C(=O)N[C@@H](CCCNC(N)=O)C(=O)Nc1ccc(COC(*)=O)cc1OCCO)C(C)C]

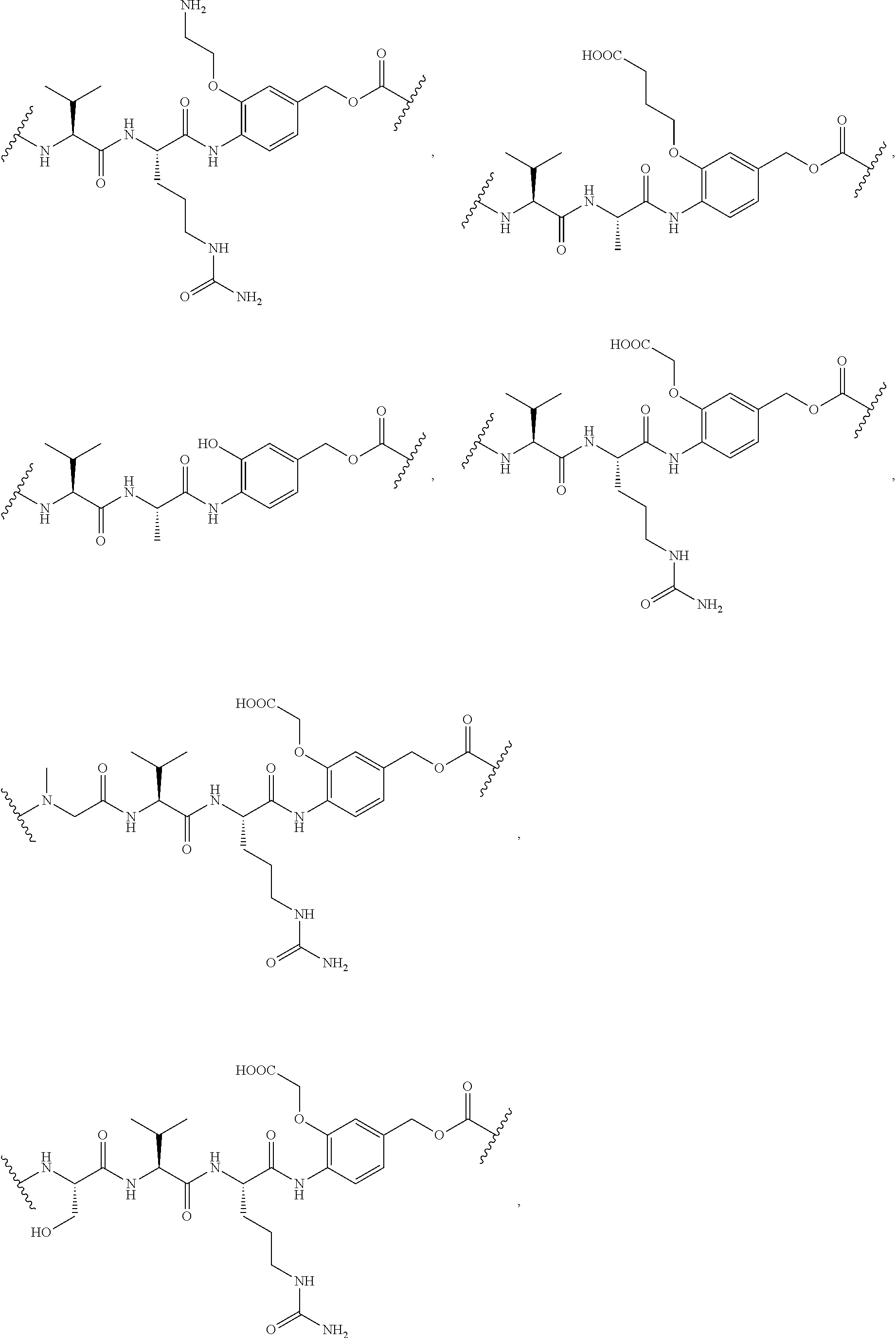 [SMILES: *N[C@H](C(=O)N[C@@H](CCCNC(N)=O)C(=O)Nc1ccc(COC(*)=O)cc1OCCN)C(C)C ; *N[C@H](C(=O)N[C@@H](C)C(=O)Nc1ccc(COC(*)=O)cc1OCCCC(=O)O)C(C)C ; *N[C@H](C(=O)N[C@@H](C)C(=O)Nc1ccc(COC(*)=O)cc1O)C(C)C ; *N[C@H](C(=O)N[C@@H](CCCNC(N)=O)C(=O)Nc1ccc(COC(*)=O)cc1OCC(=O)O)C(C)C ; *C(=O)OCc1ccc(NC(=O)[C@H](CCCNC(N)=O)NC(=O)[C@@H](NC(=O)CN(*)C)C(C)C)c(OCC(=O)O)c1 ; *N[C@@H](CO)C(=O)N[C@H](C(=O)N[C@@H](CCCNC(N)=O)C(=O)Nc1ccc(COC(*)=O)cc1OCC(=O)O)C(C)C]

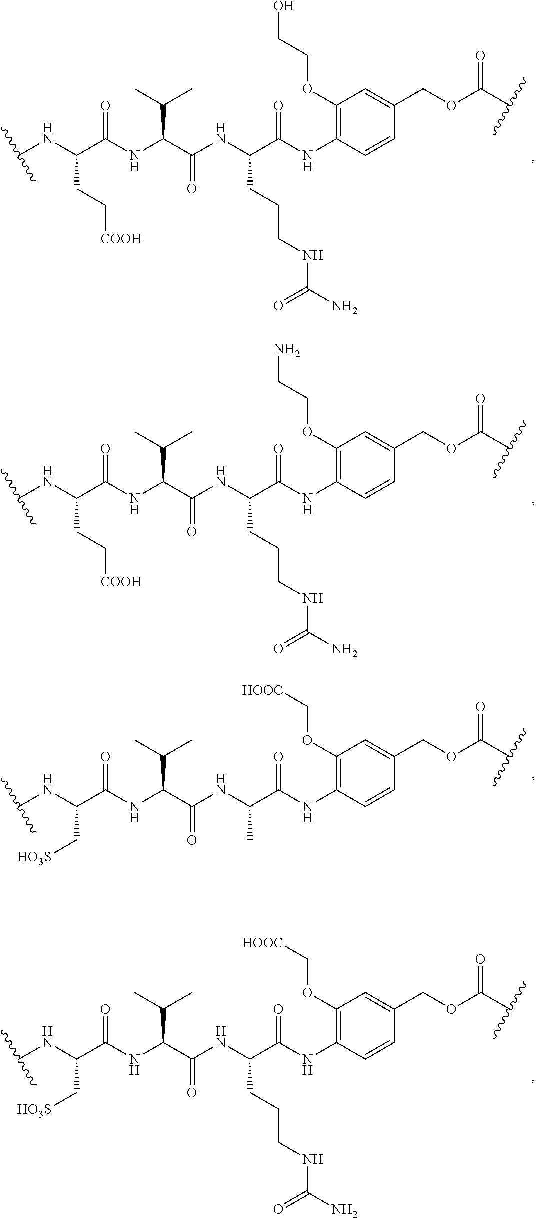

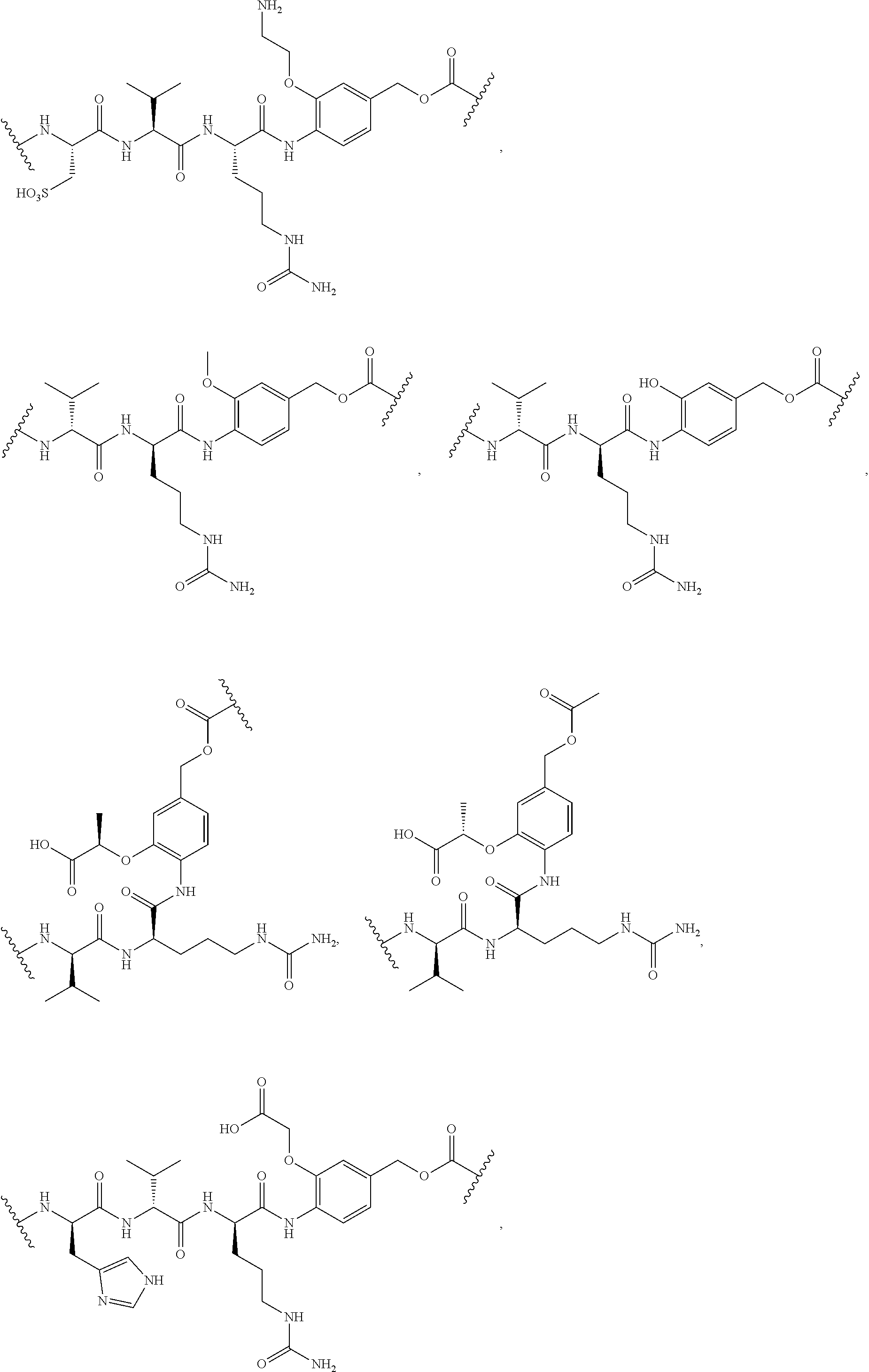 [SMILES: *N[C@@H](CS(=O)(=O)O)C(=O)N[C@H](C(=O)N[C@@H](CCCNC(N)=O)C(=O)Nc1ccc(COC(*)=O)cc1OCCN)C(C)C ; *N[C@@H](C(=O)N[C@H](CCCNC(N)=O)C(=O)Nc1ccc(COC(*)=O)cc1OC)C(C)C ; *N[C@@H](C(=O)N[C@H](CCCNC(N)=O)C(=O)Nc1ccc(COC(*)=O)cc1O)C(C)C ; *N[C@@H](C(=O)N[C@H](CCCNC(N)=O)C(=O)Nc1ccc(COC(*)=O)cc1O[C@H](C)C(=O)O)C(C)C ; *N[C@@H](C(=O)N[C@H](CCCNC(N)=O)C(=O)Nc1ccc(COC(C)=O)cc1O[C@@H](C)C(=O)O)C(C)C ; *N[C@H](Cc1c[nH]cn1)C(=O)N[C@@H](C(=O)N[C@H](CCCNC(N)=O)C(=O)Nc1ccc(COC(*)=O)cc1OCC(=O)O)C(C)C]

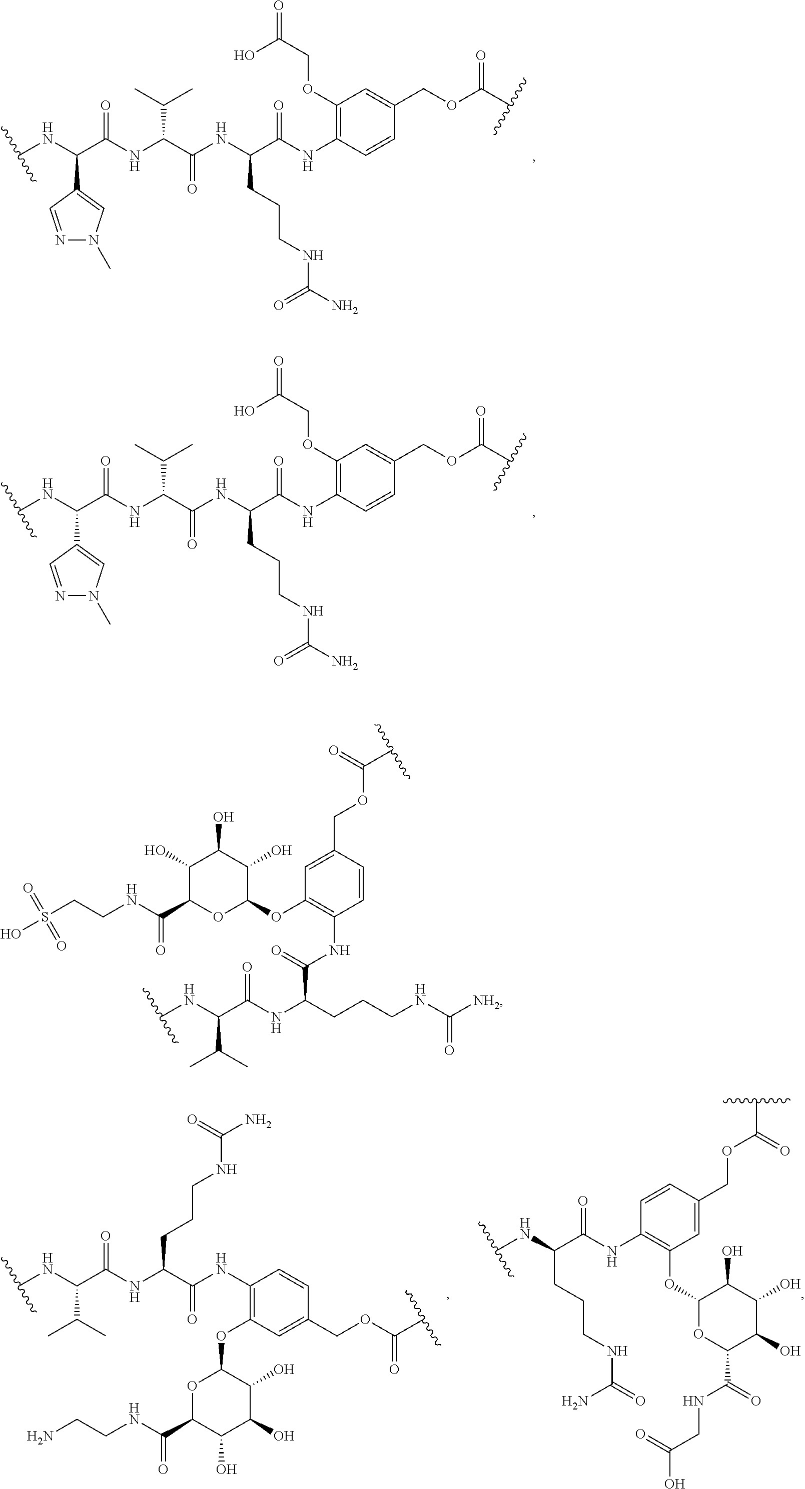

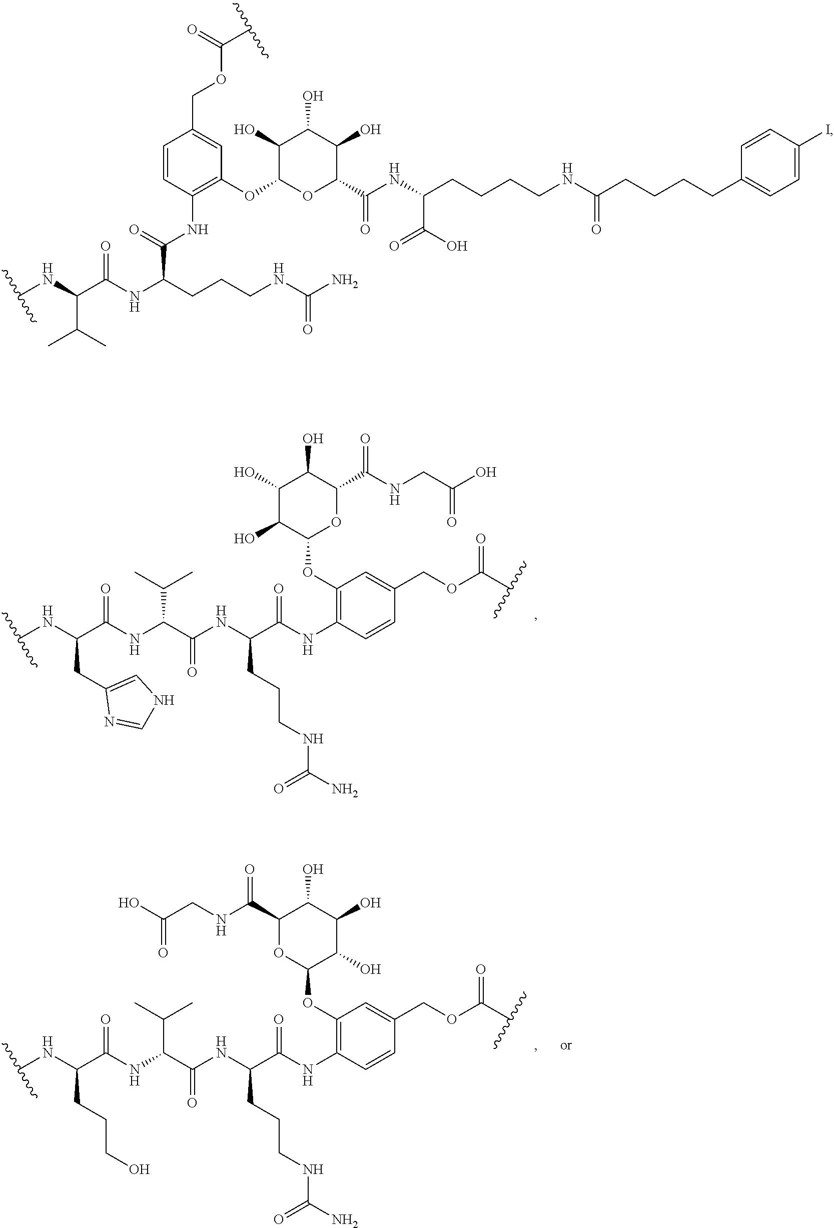 [SMILES: *N[C@@H](C(=O)N[C@H](CCCNC(N)=O)C(=O)Nc1ccc(COC(*)=O)cc1O[C@H]1O[C@@H](C(=O)N[C@H](CCCCNC(=O)CCCCc2ccc(I)cc2)C(=O)O)[C@H](O)[C@@H](O)[C@@H]1O)C(C)C ; *N[C@H](Cc1c[nH]cn1)C(=O)N[C@@H](C(=O)N[C@H](CCCNC(N)=O)C(=O)Nc1ccc(COC(*)=O)cc1O[C@H]1O[C@@H](C(=O)NCC(=O)O)[C@H](O)[C@@H](O)[C@@H]1O)C(C)C ; *N[C@H](CCCO)C(=O)N[C@@H](C(=O)N[C@H](CCCNC(N)=O)C(=O)Nc1ccc(COC(*)=O)cc1O[C@H]1O[C@@H](C(=O)NCC(=O)O)[C@H](O)[C@@H](O)[C@@H]1O)C(C)C]

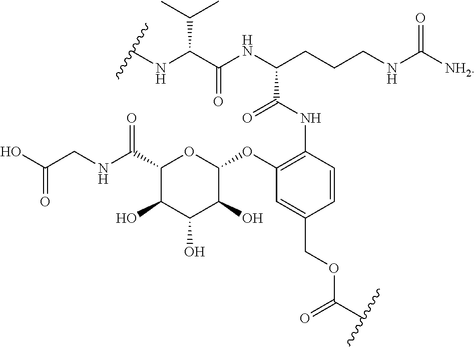 [SMILES: *N[C@@H](C(=O)N[C@H](CCCNC(N)=O)C(=O)Nc1ccc(COC(*)=O)cc1O[C@H]1O[C@@H](C(=O)NCC(=O)O)[C@H](O)[C@@H](O)[C@@H]1O)C(C)C]

In some embodiments, the linker,

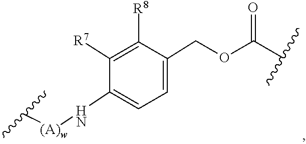

### of Formula (I) or Formula (II) is

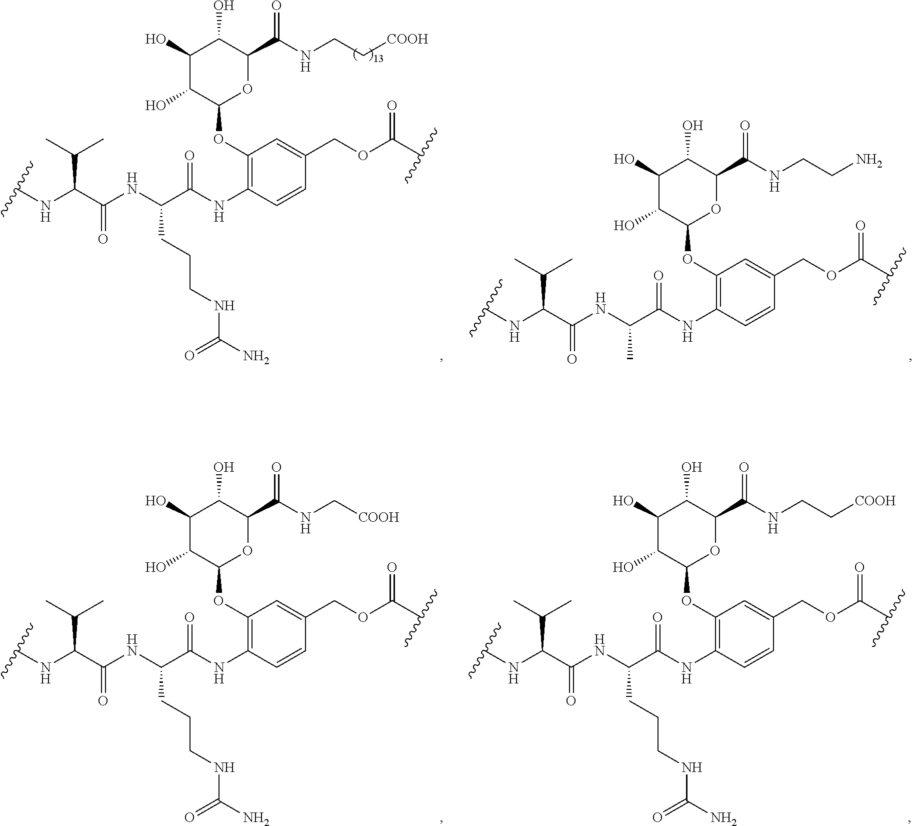

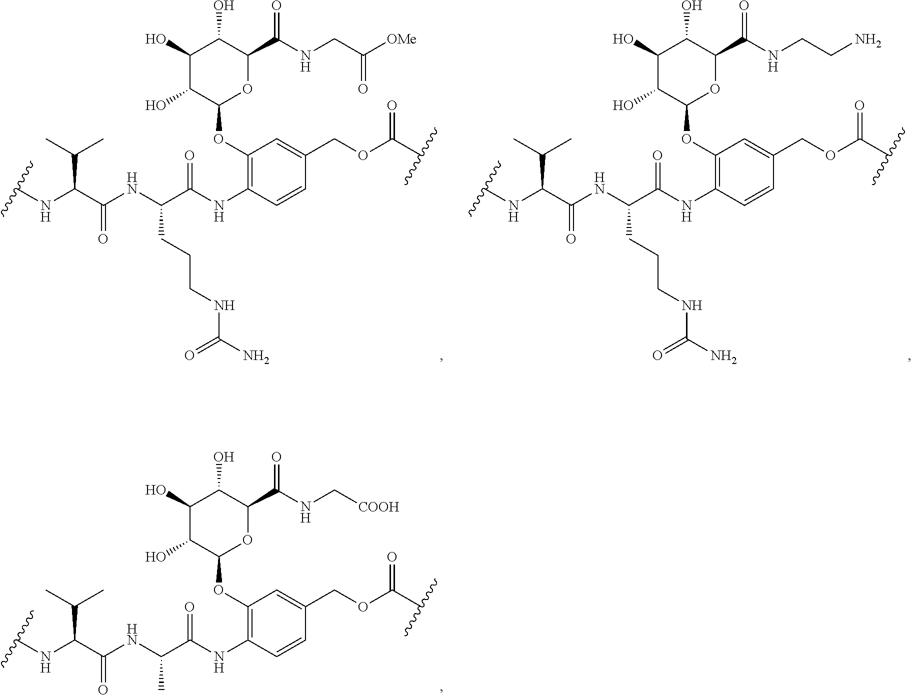 [SMILES: *N[C@H](C(=O)N[C@@H](CCCNC(N)=O)C(=O)Nc1ccc(COC(*)=O)cc1O[C@@H]1O[C@H](C(=O)NCC(=O)OC)[C@@H](O)[C@H](O)[C@H]1O)C(C)C ; *N[C@H](C(=O)N[C@@H](CCCNC(N)=O)C(=O)Nc1ccc(COC(*)=O)cc1O[C@@H]1O[C@H](C(=O)NCCN)[C@@H](O)[C@H](O)[C@H]1O)C(C)C ; *N[C@H](C(=O)N[C@@H](C)C(=O)Nc1ccc(COC(*)=O)cc1O[C@@H]1O[C@H](C(=O)NCC(=O)O)[C@@H](O)[C@H](O)[C@H]1O)C(C)C]

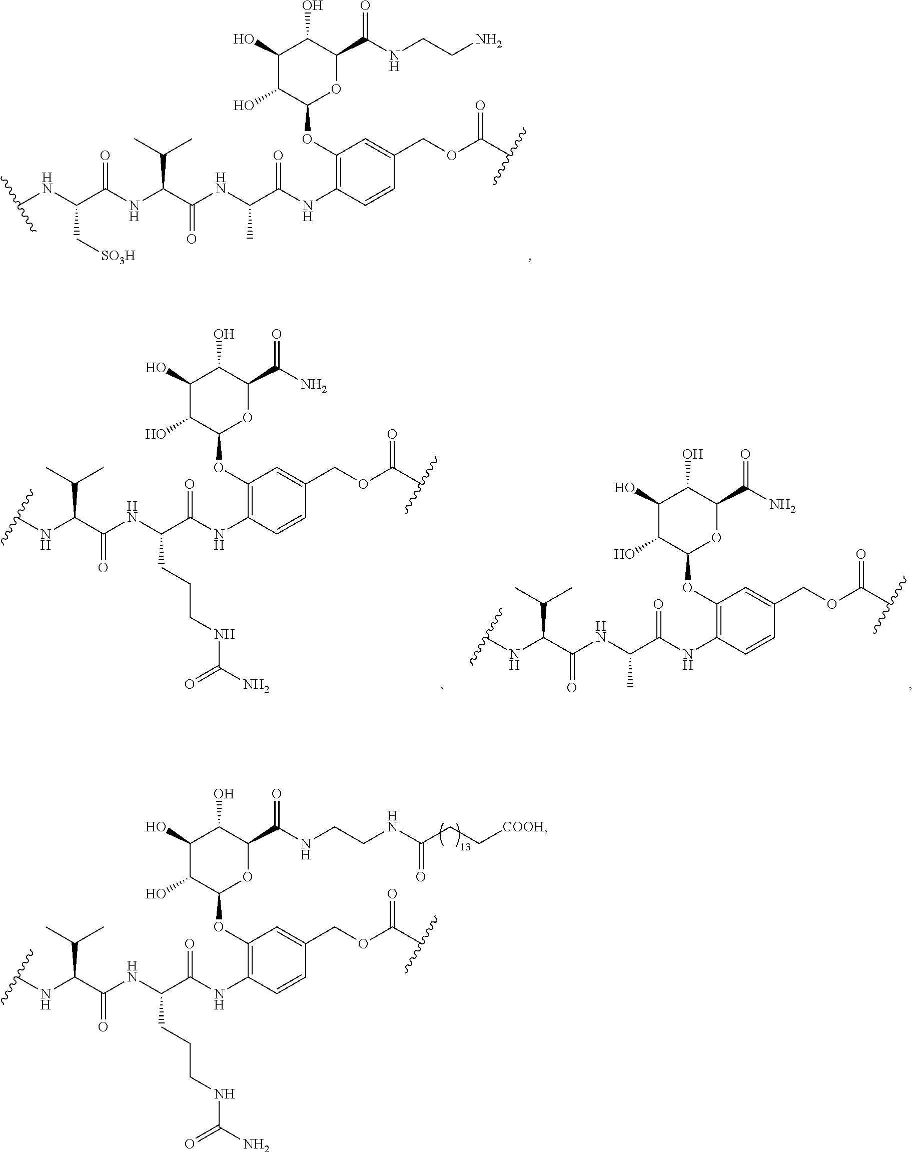

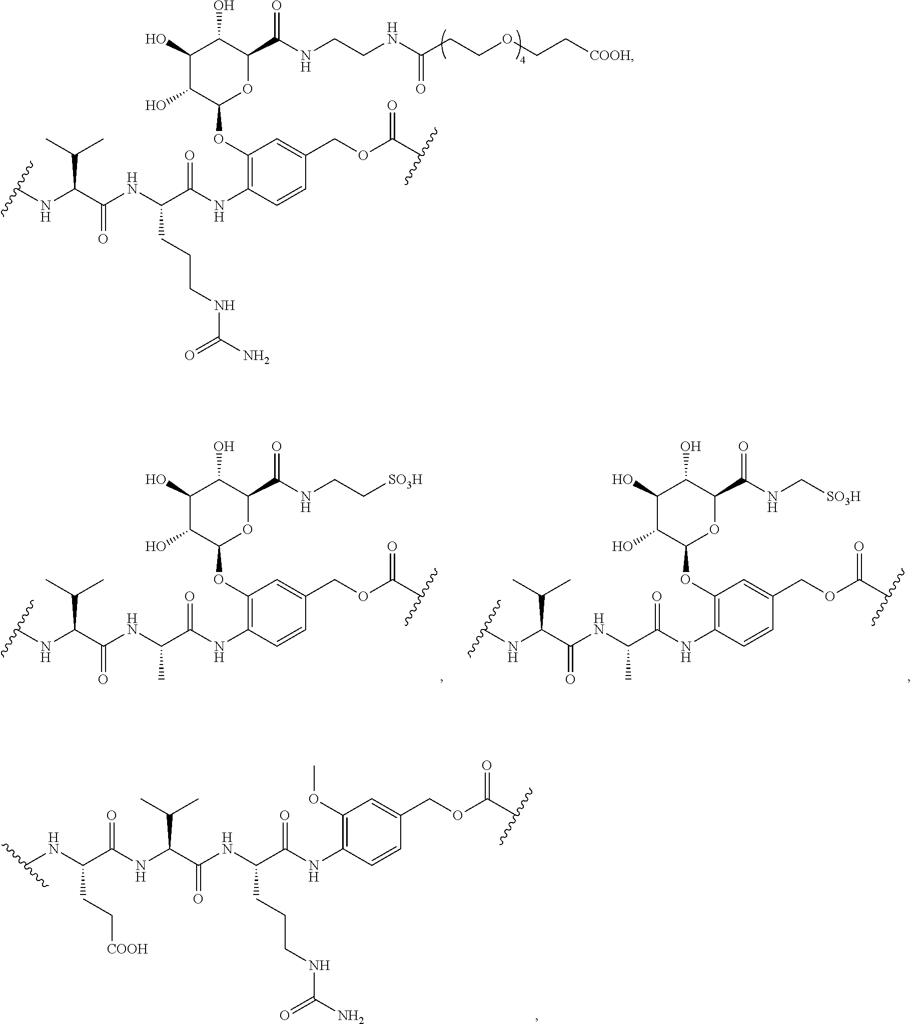

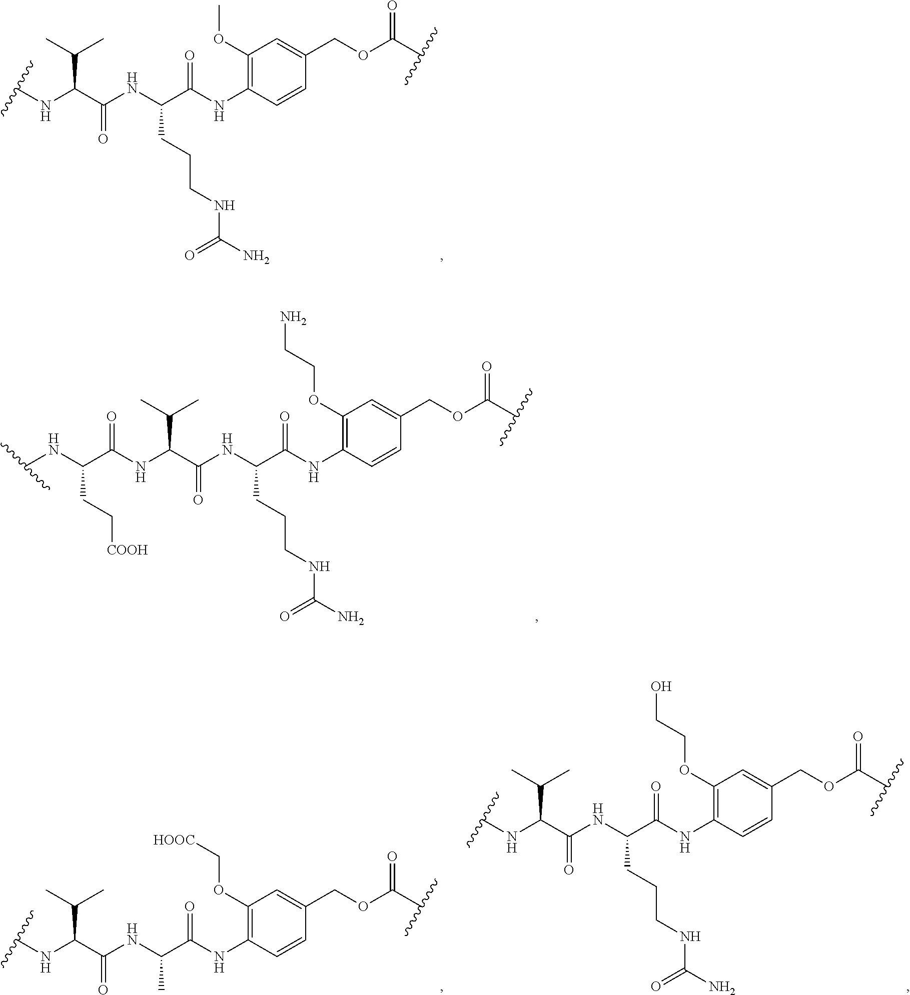 [SMILES: *N[C@H](C(=O)N[C@@H](CCCNC(N)=O)C(=O)Nc1ccc(COC(*)=O)cc1OC)C(C)C ; *N[C@@H](CCC(=O)O)C(=O)N[C@H](C(=O)N[C@@H](CCCNC(N)=O)C(=O)Nc1ccc(COC(*)=O)cc1OCCN)C(C)C ; *N[C@H](C(=O)N[C@@H](C)C(=O)Nc1ccc(COC(*)=O)cc1OCC(=O)O)C(C)C ; *N[C@H](C(=O)N[C@@H](CCCNC(N)=O)C(=O)Nc1ccc(COC(*)=O)cc1OCCO)C(C)C]

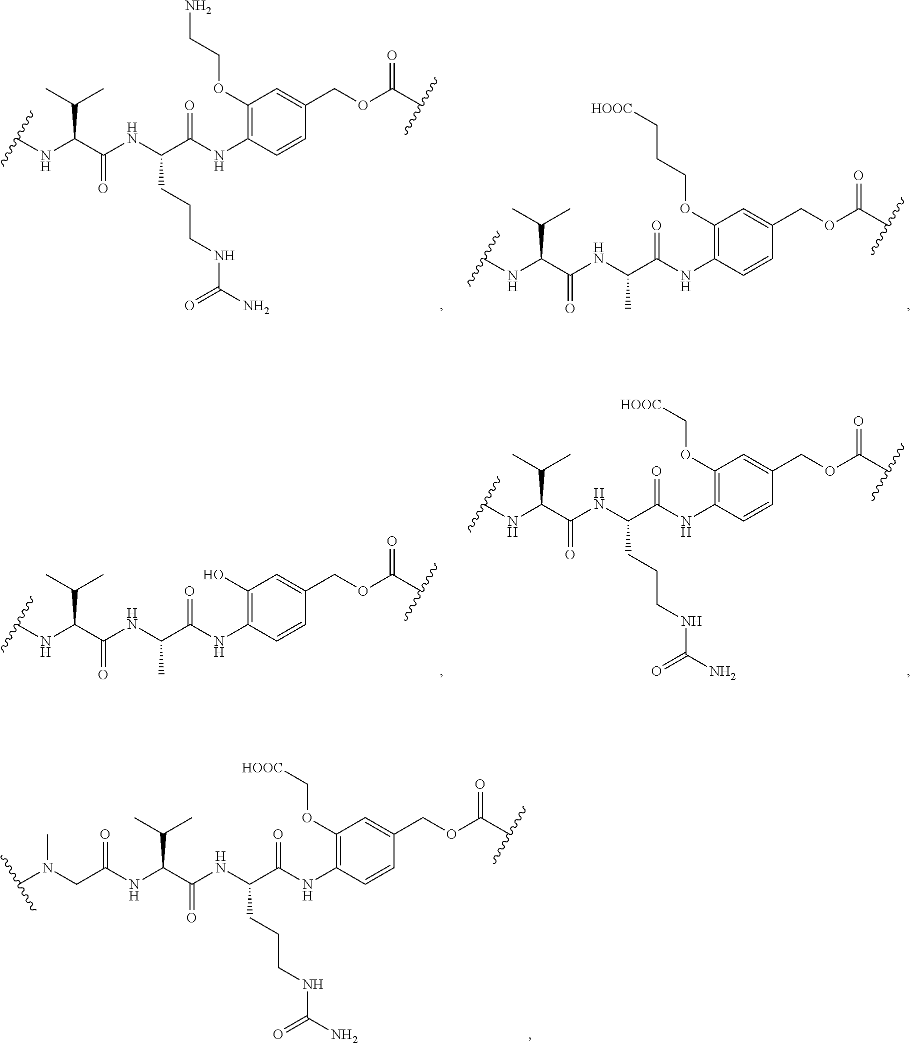 [SMILES: *N[C@H](C(=O)N[C@@H](CCCNC(N)=O)C(=O)Nc1ccc(COC(*)=O)cc1OCCN)C(C)C ; *N[C@H](C(=O)N[C@@H](C)C(=O)Nc1ccc(COC(*)=O)cc1OCCCC(=O)O)C(C)C ; *N[C@H](C(=O)N[C@@H](C)C(=O)Nc1ccc(COC(*)=O)cc1O)C(C)C ; *N[C@H](C(=O)N[C@@H](CCCNC(N)=O)C(=O)Nc1ccc(COC(*)=O)cc1OCC(=O)O)C(C)C ; *C(=O)OCc1ccc(NC(=O)[C@H](CCCNC(N)=O)NC(=O)[C@@H](NC(=O)CN(*)C)C(C)C)c(OCC(=O)O)c1]

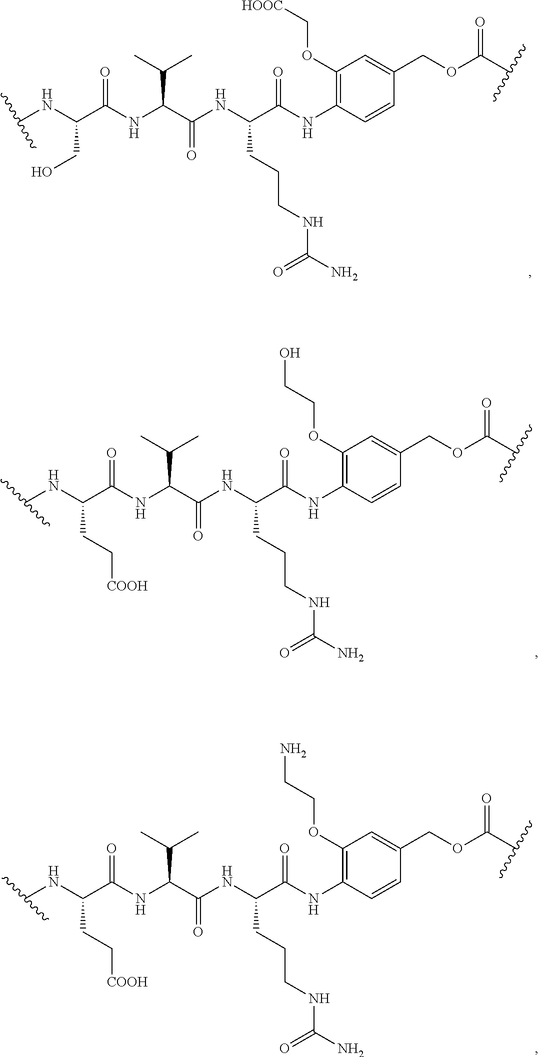 [SMILES: *N[C@@H](CO)C(=O)N[C@H](C(=O)N[C@@H](CCCNC(N)=O)C(=O)Nc1ccc(COC(*)=O)cc1OCC(=O)O)C(C)C ; *N[C@@H](CCC(=O)O)C(=O)N[C@H](C(=O)N[C@@H](CCCNC(N)=O)C(=O)Nc1ccc(COC(*)=O)cc1OCCO)C(C)C ; *N[C@@H](CCC(=O)O)C(=O)N[C@H](C(=O)N[C@@H](CCCNC(N)=O)C(=O)Nc1ccc(COC(*)=O)cc1OCCN)C(C)C]

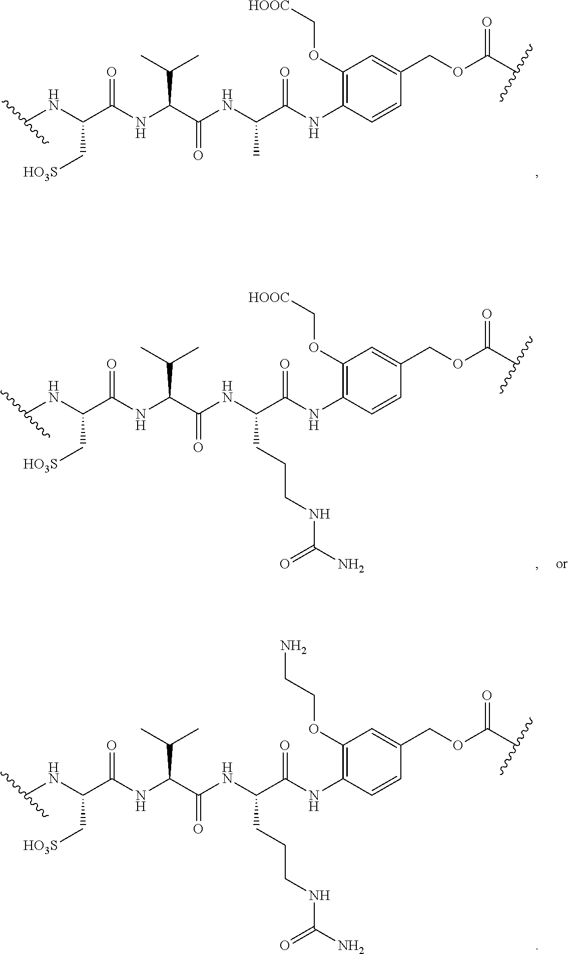 [SMILES: *N[C@@H](CS(=O)(=O)O)C(=O)N[C@H](C(=O)N[C@@H](C)C(=O)Nc1ccc(COC(*)=O)cc1OCC(=O)O)C(C)C ; *N[C@@H](CS(=O)(=O)O)C(=O)N[C@H](C(=O)N[C@@H](CCCNC(N)=O)C(=O)Nc1ccc(COC(*)=O)cc1OCC(=O)O)C(C)C ; *N[C@@H](CS(=O)(=O)O)C(=O)N[C@H](C(=O)N[C@@H](CCCNC(N)=O)C(=O)Nc1ccc(COC(*)=O)cc1OCCN)C(C)C]

In some embodiments, the linker,

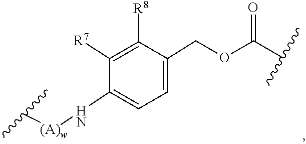

### of Formula (I) or Formula (II) is:

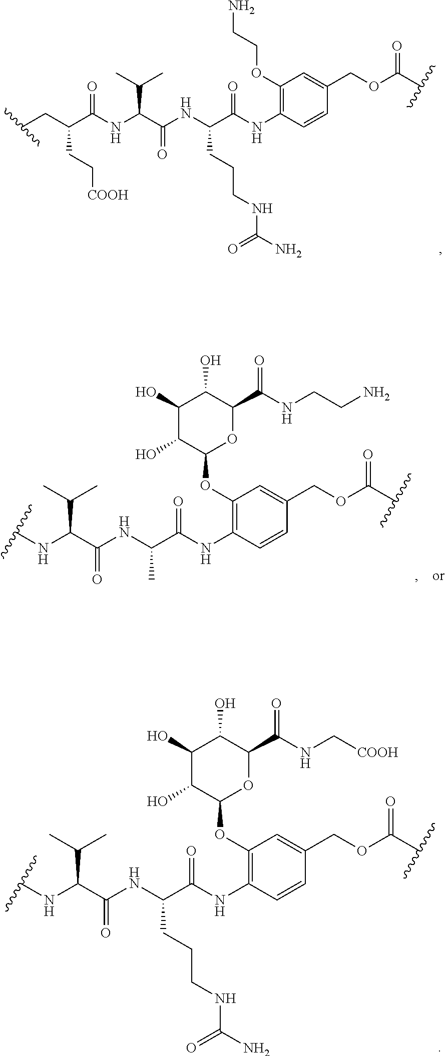 [SMILES: *C[C@@H](CCC(=O)O)C(=O)N[C@H](C(=O)N[C@@H](CCCNC(N)=O)C(=O)Nc1ccc(COC(*)=O)cc1OCCN)C(C)C ; *N[C@H](C(=O)N[C@@H](C)C(=O)Nc1ccc(COC(*)=O)cc1O[C@@H]1O[C@H](C(=O)NCCN)[C@@H](O)[C@H](O)[C@H]1O)C(C)C ; *N[C@H](C(=O)N[C@@H](CCCNC(N)=O)C(=O)Nc1ccc(COC(*)=O)cc1O[C@@H]1O[C@H](C(=O)NCC(=O)O)[C@@H](O)[C@H](O)[C@H]1O)C(C)C]

Any combination of the groups described above for the various variables is contemplated herein. Throughout the specification, groups and substituents thereof are chosen by one skilled in the field to provide stable moieties and compounds.

### Representative Conjugates

In some embodiments, the compound of Formula (I) is Compound 1.

In some embodiments, the compound of Formula (I) is Compound 2.

In some embodiments, the compound of Formula (I) is Compound 3.

In some embodiments, the compound of Formula (I) is Compound 4.

In some embodiments, the compound of Formula (I) is Compound 5.

In some embodiments, the compound of Formula (I) is Compound 6.

In some embodiments, the compound of Formula (I) is Compound 7.

In some embodiments, the compound of Formula (I) is Compound 8.

In some embodiments, the compound of Formula (I) is Compound 9.

In some embodiments, the compound of Formula (I) is Compound 10.

In some embodiments, the compound of Formula (I) is Compound 11.

In some embodiments, the compound of Formula (I) is Compound 12.

In some embodiments, the compound of Formula (I) is Compound 13.

In some embodiments, the compound of Formula (I) is Compound 14.

In some embodiments, the compound of Formula (I) is Compound 15.

In some embodiments, the compound of Formula (I) is Compound 16.

In some embodiments, the compound of Formula (I) is Compound 17.

In some embodiments, the compound of Formula (I) is Compound 18.

In some embodiments, the compound of Formula (I) is Compound 19.

In some embodiments, the compound of Formula (I) is Compound 20.

In some embodiments, the compound of Formula (I) is Compound 21.

In some embodiments, the compound of Formula (I) is Compound 22.

In some embodiments, the compound of Formula (I) is Compound 23.

In some embodiments, the compound of Formula (I) is Compound 24.

In some embodiments, the compound of Formula (I) is Compound 25.

In some embodiments, the compound of Formula (I) is Compound 26.

In some embodiments, the compound of Formula (I) is Compound 27.

In some embodiments, the compound of Formula (I) is Compound 28.

In some embodiments, the compound of Formula (I) is Compound 29.

In some embodiments, the compound of Formula (I) is Compound 30.

In some embodiments, the compound of Formula (I) is Compound 31.

In some embodiments, the compound of Formula (I) is Compound 32.

In some embodiments, the compound of Formula (I) is Compound 33.

In some embodiments, the compound of Formula (I) is Compound 34.

In some embodiments, the compound of Formula (I) is Compound 35.

In some embodiments, the compound of Formula (I) is Compound 36.

In some embodiments, the compound of Formula (I) is Compound 37.

In some embodiments, the compound of Formula (I) is Compound 38.

In some embodiments, the compound of Formula (I) is Compound 39.

In some embodiments, the compound of Formula (I) is Compound 40.

In some embodiments, the compound of Formula (I) is Compound 41.

In some embodiments, the compound of Formula (I) is Compound 42.

In some embodiments, the compound of Formula (I) is Compound 43.

In some embodiments, the compound of Formula (I) is Compound 44.

In some embodiments, the compound of Formula (I) is Compound 45.

In some embodiments, the compound of Formula (I) is Compound 46.

In some embodiments, the compound of Formula (I) is Compound 47.

In some embodiments, the compound of Formula (I) is Compound 48.

In some embodiments, the compound of Formula (I) is Compound 49.

In some embodiments, the compound of Formula (I) is Compound 50.

In some embodiments, the compound of Formula (I) is Compound 51.

In some embodiments, the compound of Formula (I) is Compound 52.

In some embodiments, the compound of Formula (I) is Compound 53.

In some embodiments, the compound of Formula (I) is Compound 54.

In some embodiments, the compound of Formula (I) is Compound 55.

In some embodiments, the compound of Formula (I) is Compound 56.

In some embodiments, the compound of Formula (I) is Compound 59.

In some embodiments, the compound of Formula (I) is Compound 60.

In some embodiments, the compound of Formula (I) is Compound 61.

In some embodiments, the compound of Formula (I) is Compound 62.

In some embodiments, the compound of Formula (I) is Compound 65.

In some embodiments, the compound of Formula (I) is Compound 67.

In some embodiments, the compound of Formula (I) is Compound 69.

In some embodiments, the compound of Formula (I) is Compound 71.

In some embodiments, the compound of Formula (I) is Compound 72.

In some embodiments, the compound of Formula (I) is Compound 73.

In some embodiments, the compound of Formula (I) is Compound 74.

In some embodiments, the compound of Formula (I) is Compound 75.

In some embodiments, the compound of Formula (I) is Compound 76.

In some embodiments, the compound of Formula (I) is Compound 77.

In some embodiments, the compound of Formula (I) is Compound 78.

In some embodiments, the compound of Formula (I) is Compound 83.

In some embodiments, the compound of Formula (I) is Compound 86.

In some embodiments, the compound of Formula (I) is Compound 87.

In some embodiments, the compound of Formula (I) is Compound 88.

In some embodiments, the compound of Formula (I) is Compound 89.

In some embodiments, the compound of Formula (I) is Compound 90.

In some embodiments, the compound of Formula (I) is Compound 91.

In some embodiments, the compound of Formula (I) is Compound 198.

In some embodiments, the compound of Formula (I) is Compound 199.

In some embodiments, the compound of Formula (I) is Compound 200.

In some embodiments, the compound of Formula (I) is Compound 201.

In some embodiments, the compound of Formula (I) is Compound 202.

In some embodiments, the compound of Formula (I) is Compound 203.

In some embodiments, the compound of Formula (I) is Compound 204.

In some embodiments, the compound of Formula (I) is Compound 208.

In some embodiments, the compound of Formula (I) is Compound 209.

In some embodiments, the compound of Formula (I) is Compound 210.

In some embodiments, the compound of Formula (I) is Compound 211.

In some embodiments, the compound of Formula (I) is Compound 212.

In some embodiments, the compound of Formula (I) is Compound 213.

In some embodiments, the compound of Formula (I) is Compound 214.

In some embodiments, the compound of Formula (I) is selected from:

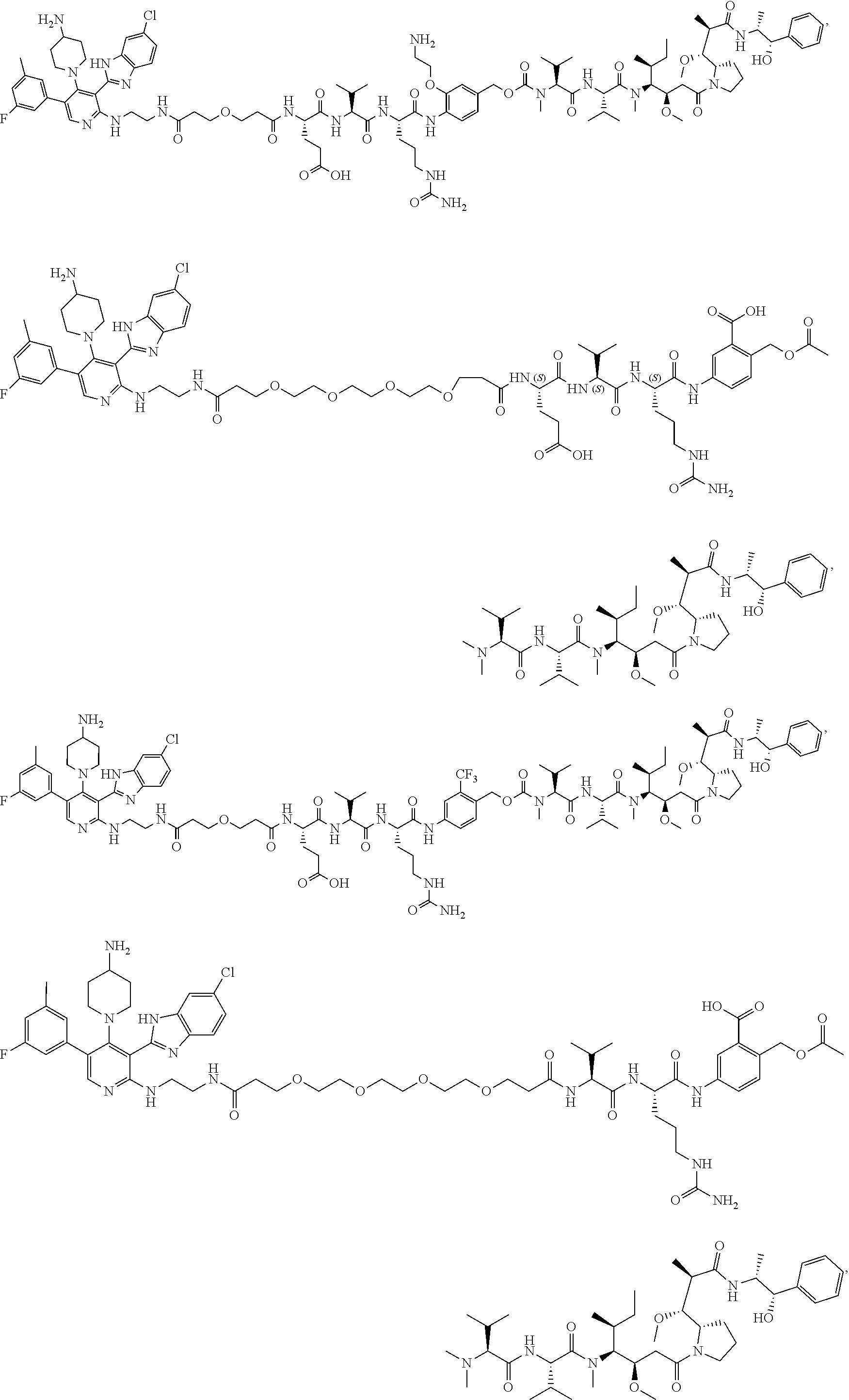

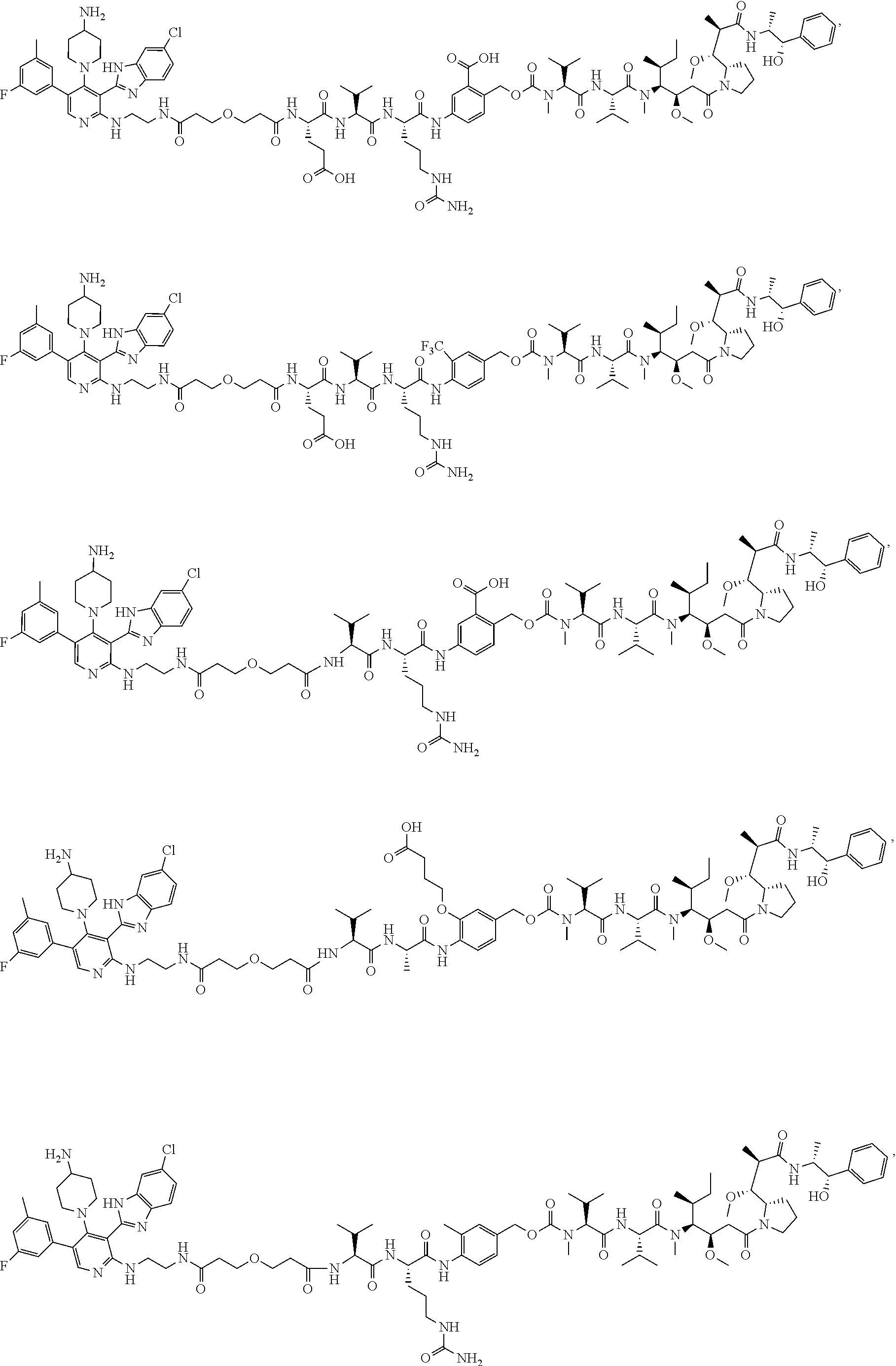

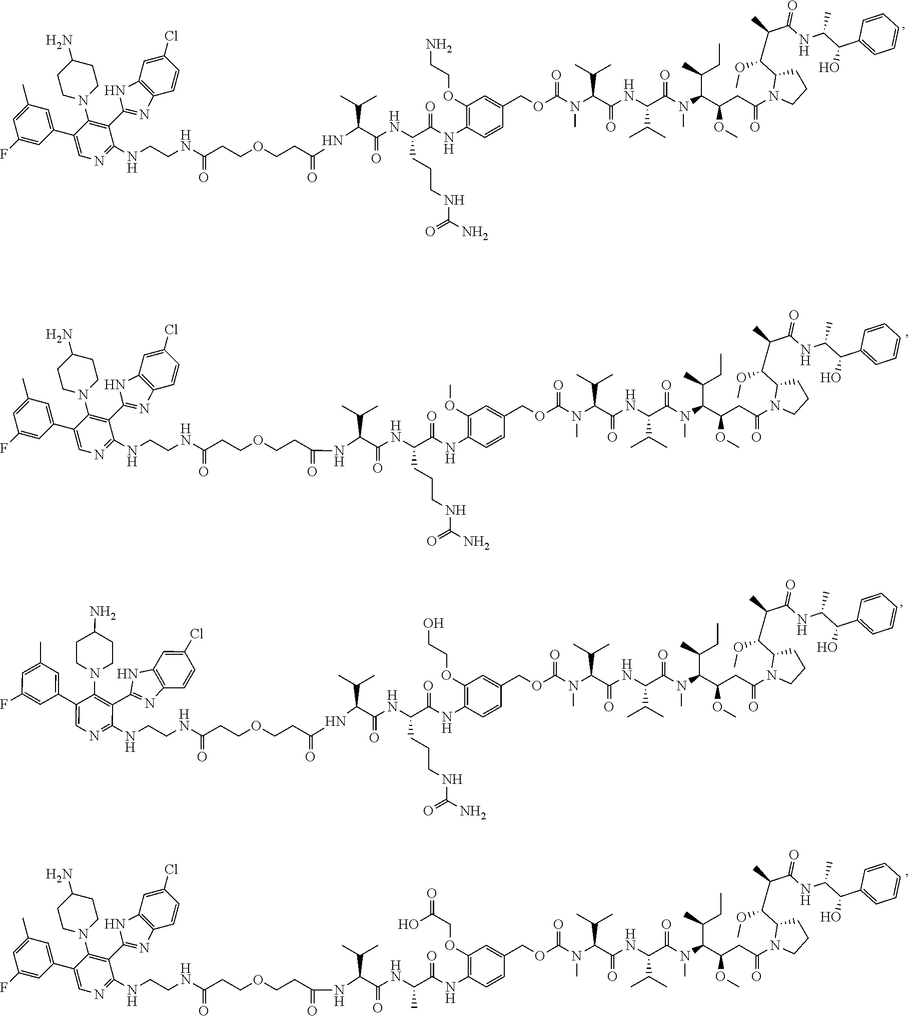

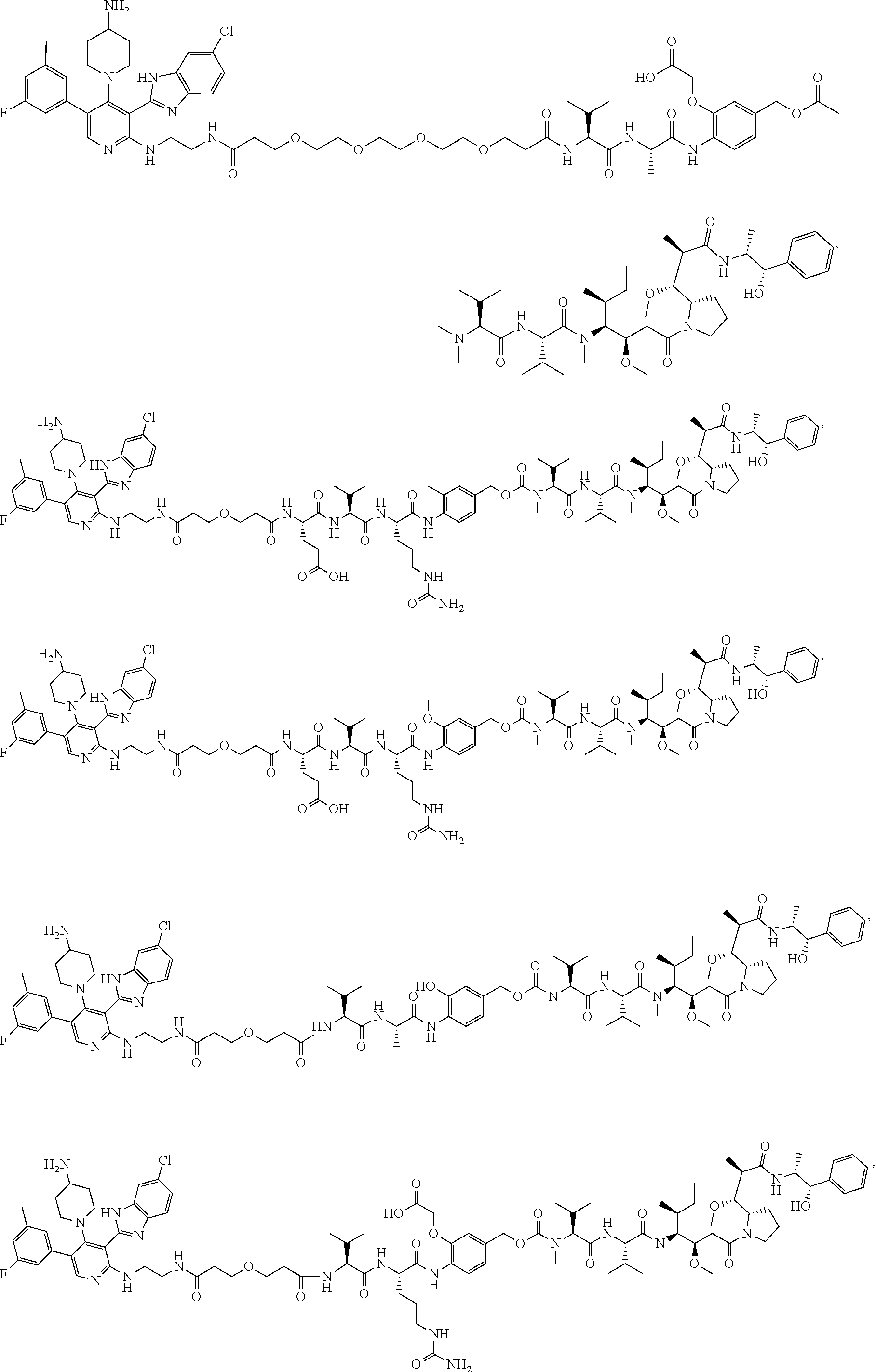

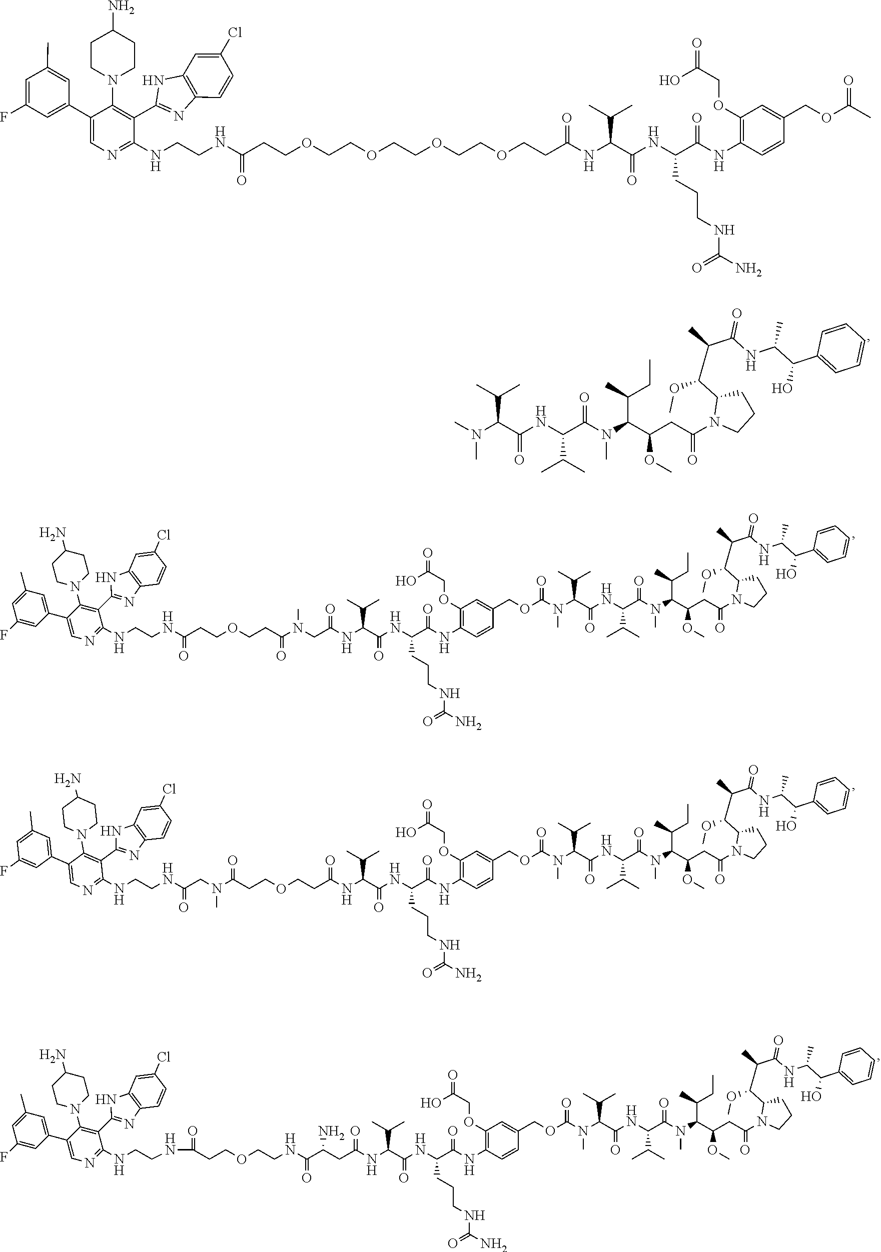

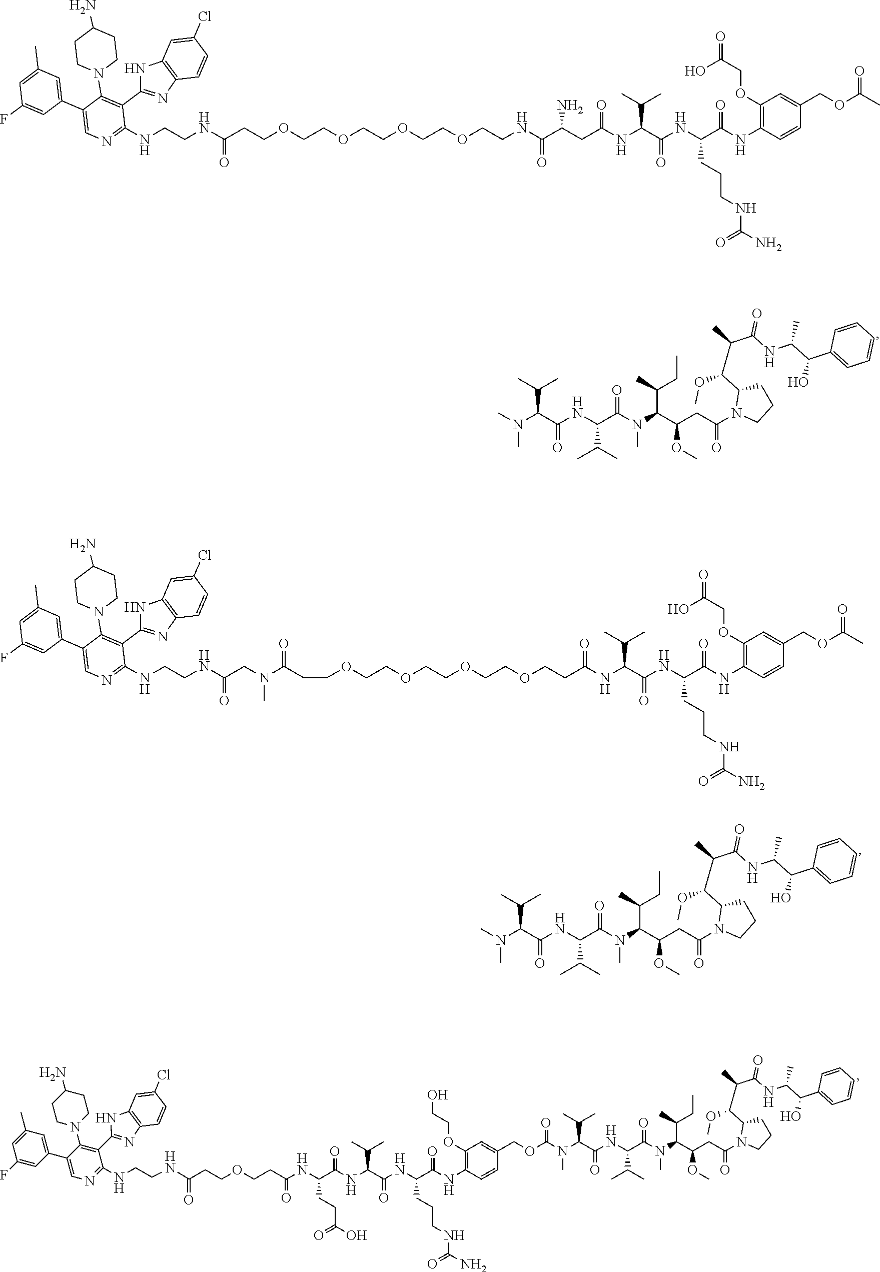

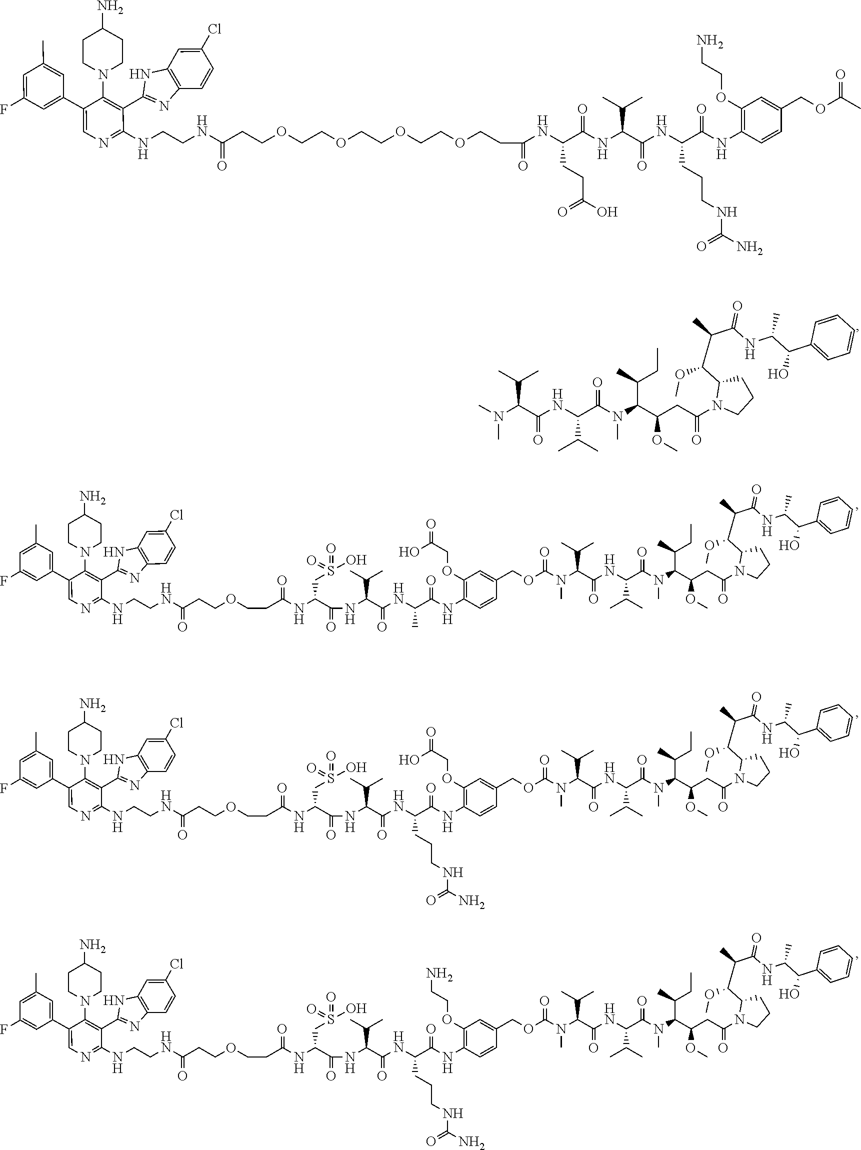

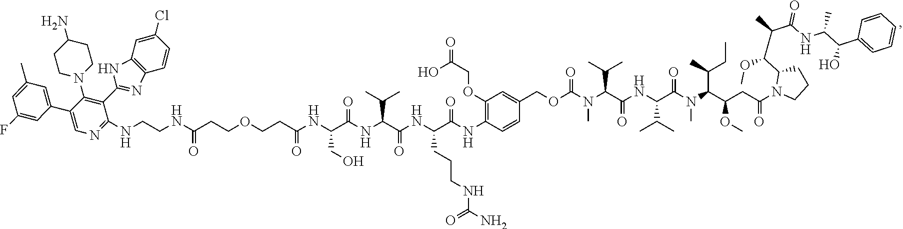 [SMILES: CC[C@H](C)[C@@H]([C@@H](CC(=O)N1CCC[C@H]1[C@H](OC)[C@@H](C)C(=O)N[C@H](C)[C@@H](O)c1ccccc1)OC)N(C)C(=O)[C@@H](NC(=O)[C@H](C(C)C)N(C)C(=O)OCc1ccc(NC(=O)[C@H](CCCNC(N)=O)NC(=O)[C@@H](NC(=O)[C@H](CO)NC(=O)CCOCCC(=O)NCCNc2ncc(-c3cc(C)cc(F)c3)c(N3CCC(N)CC3)c2-c2nc3ccc(Cl)cc3[nH]2)C(C)C)c(OCC(=O)O)c1)C(C)C ; ","]

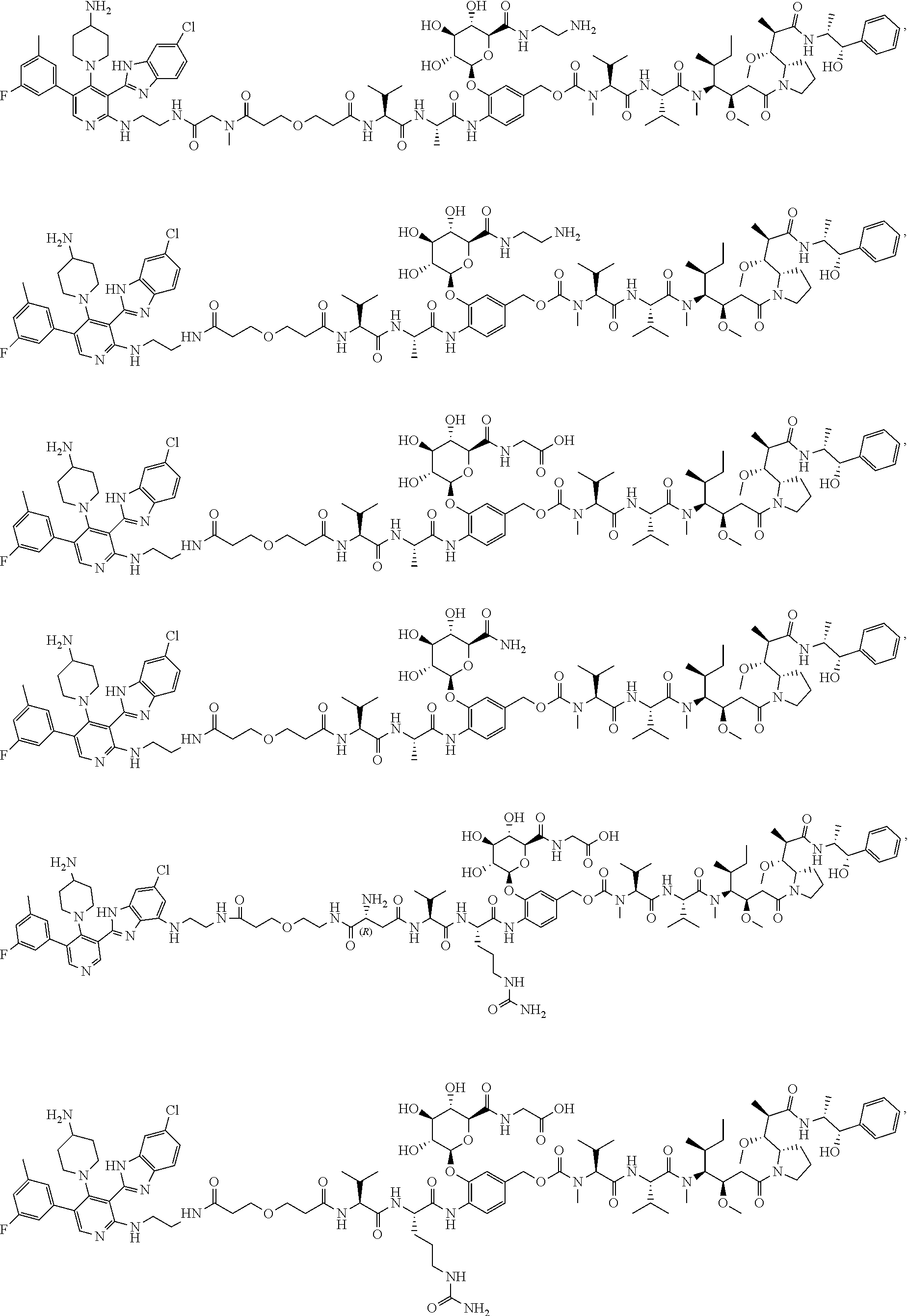

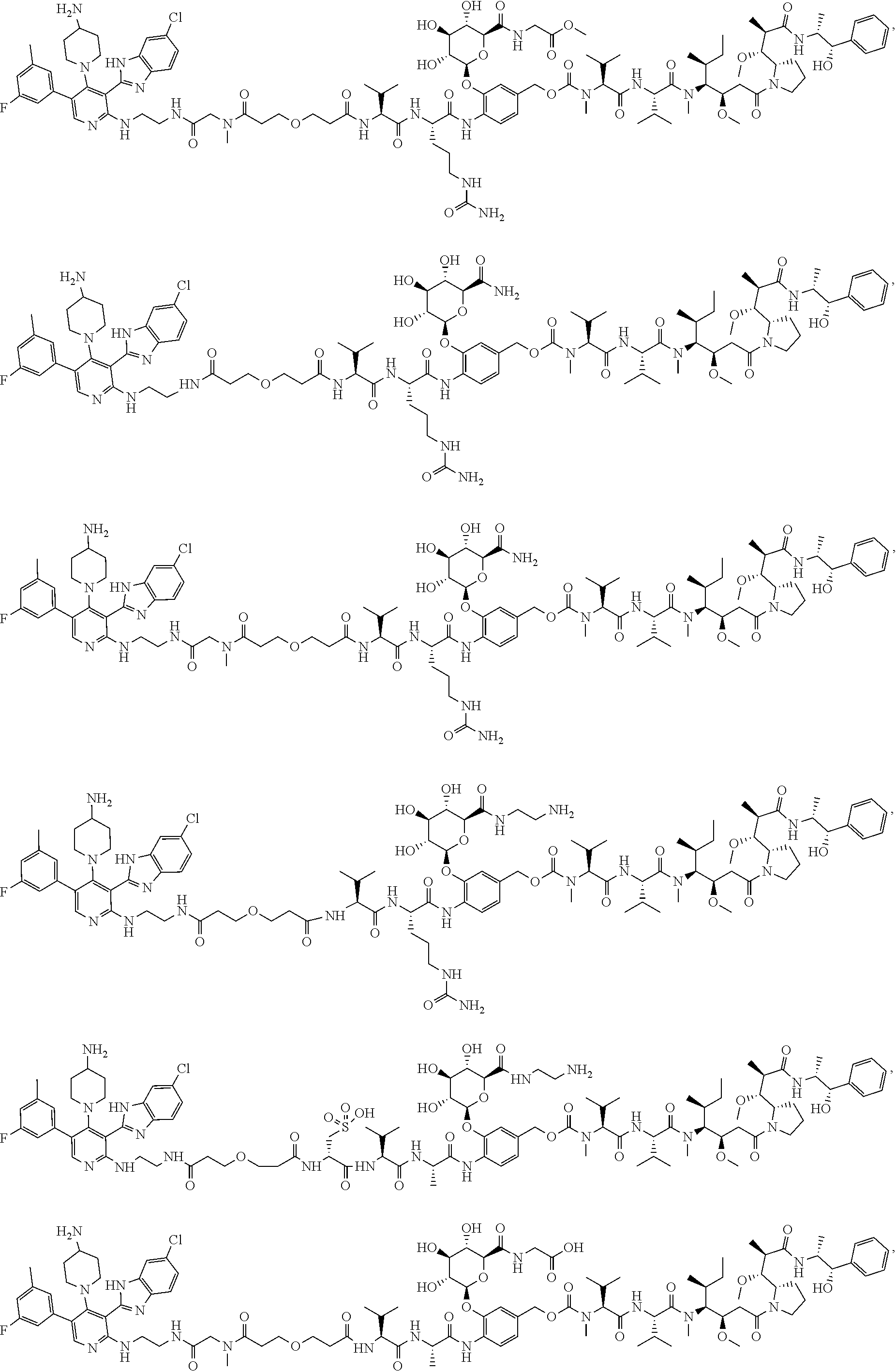

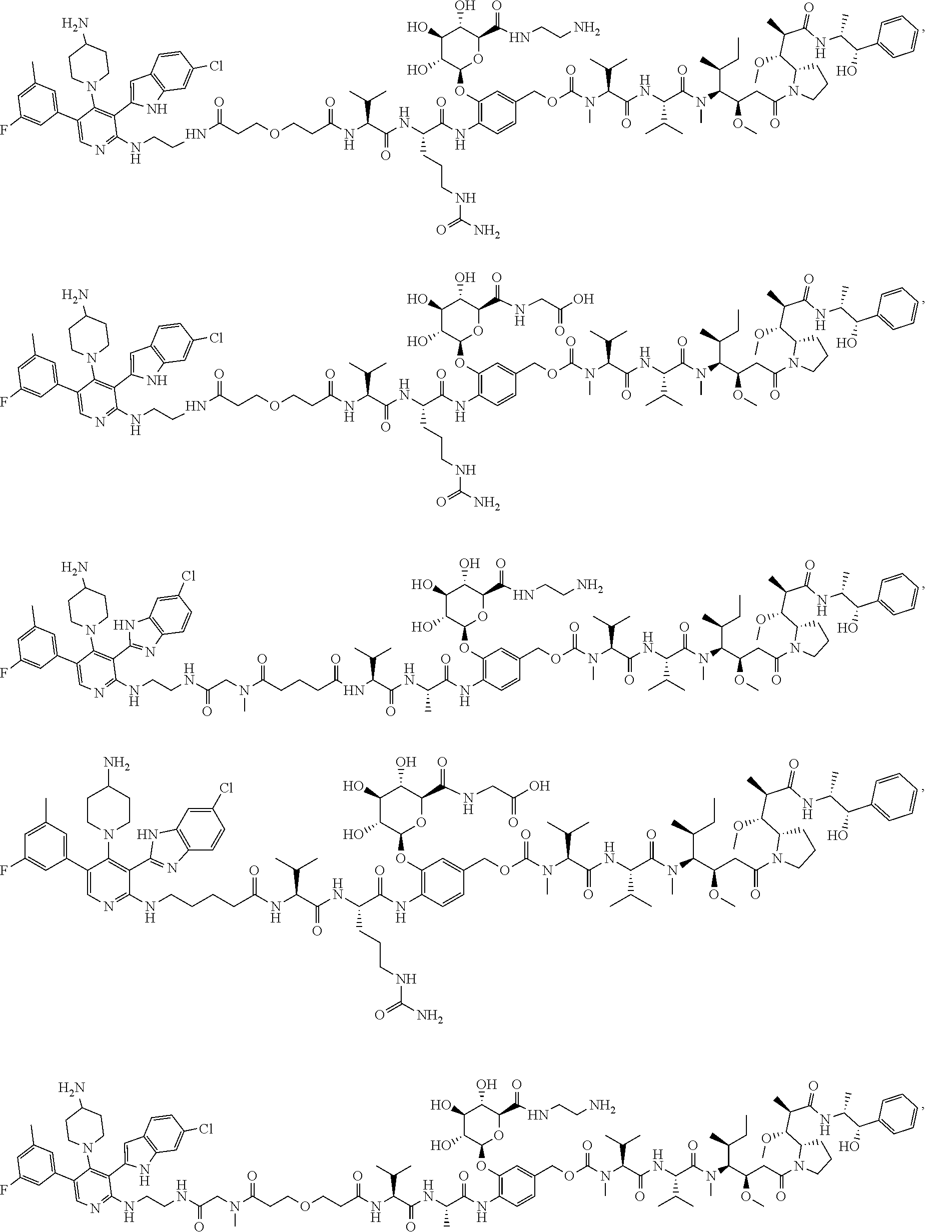

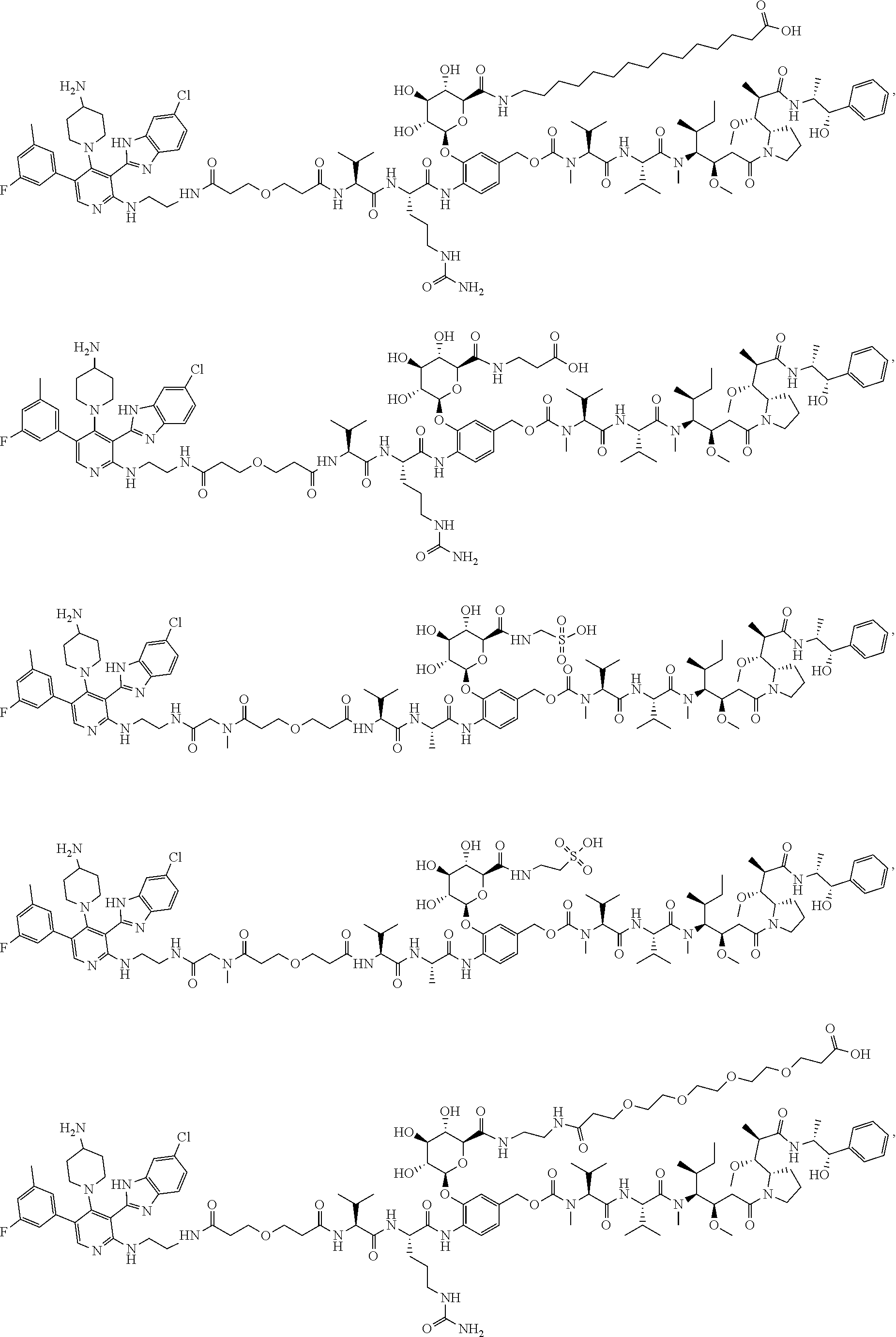

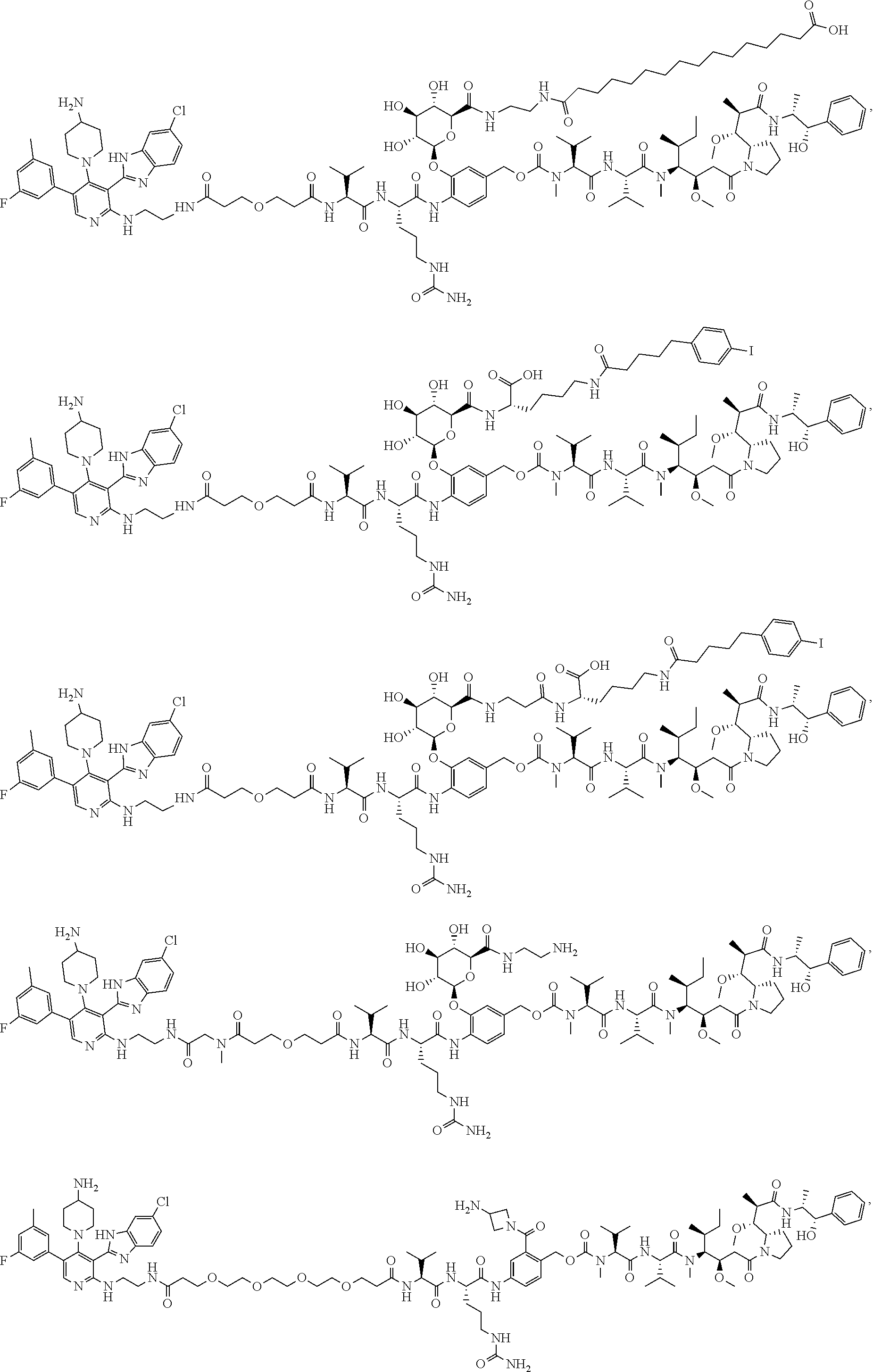

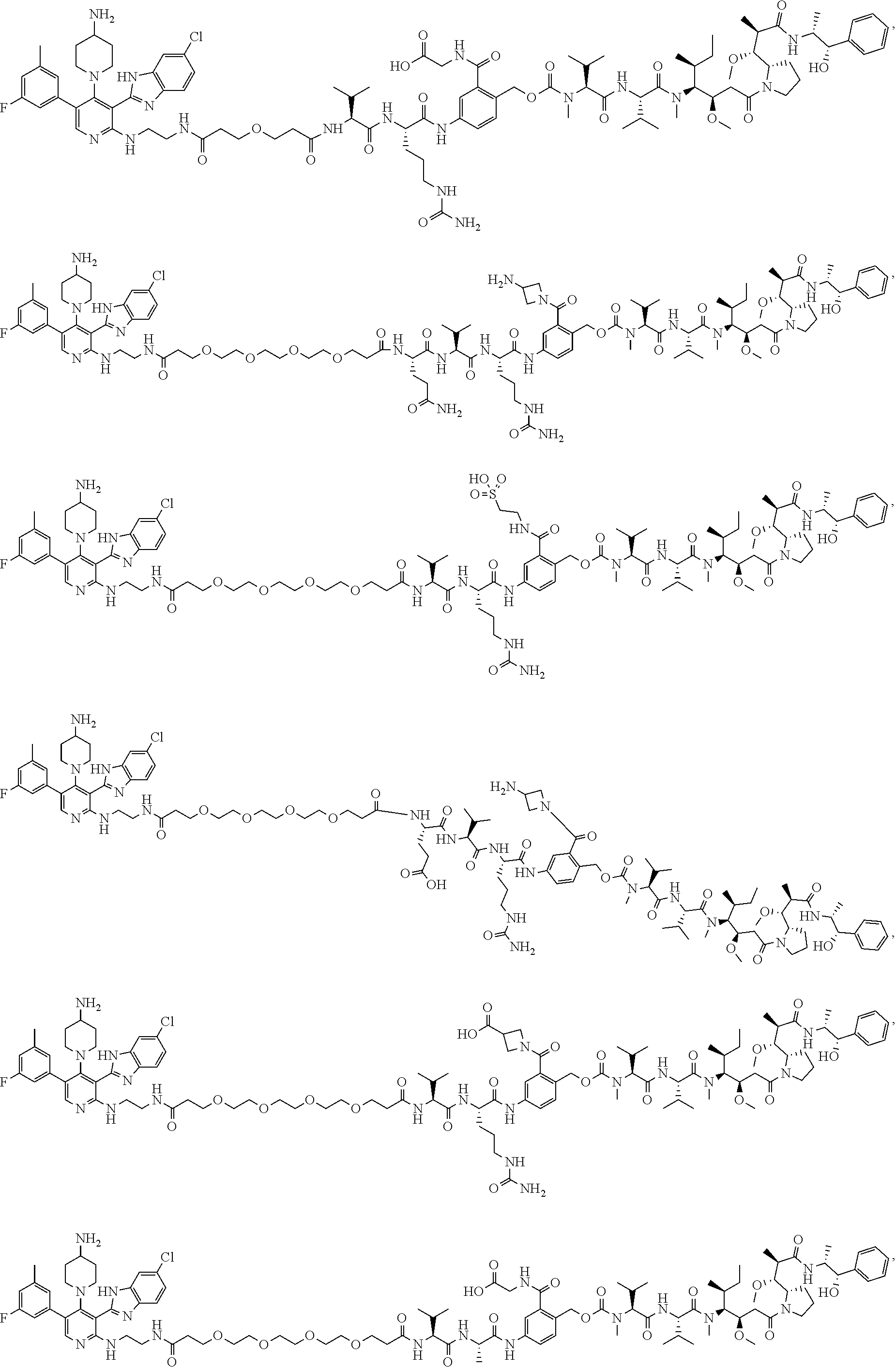

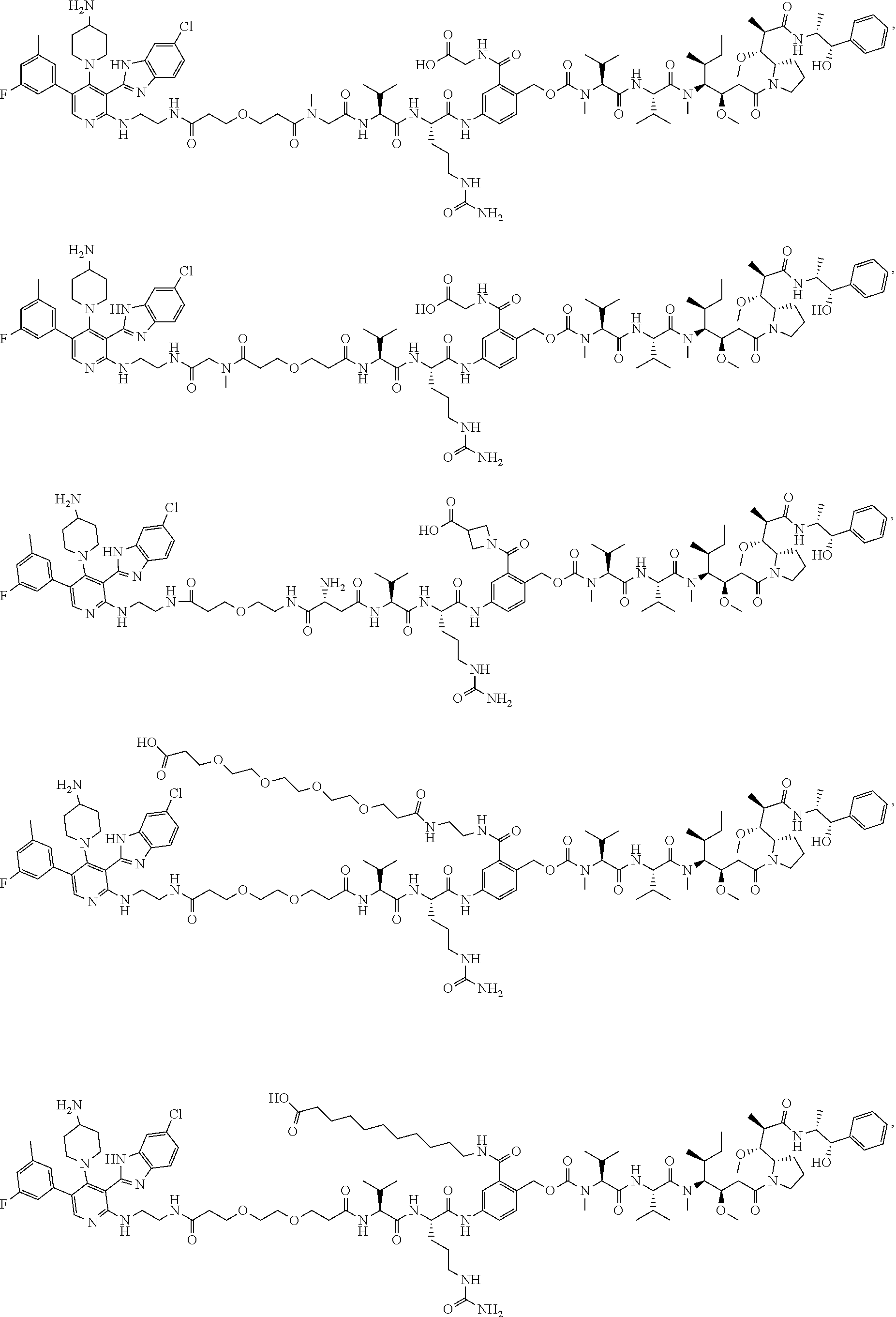

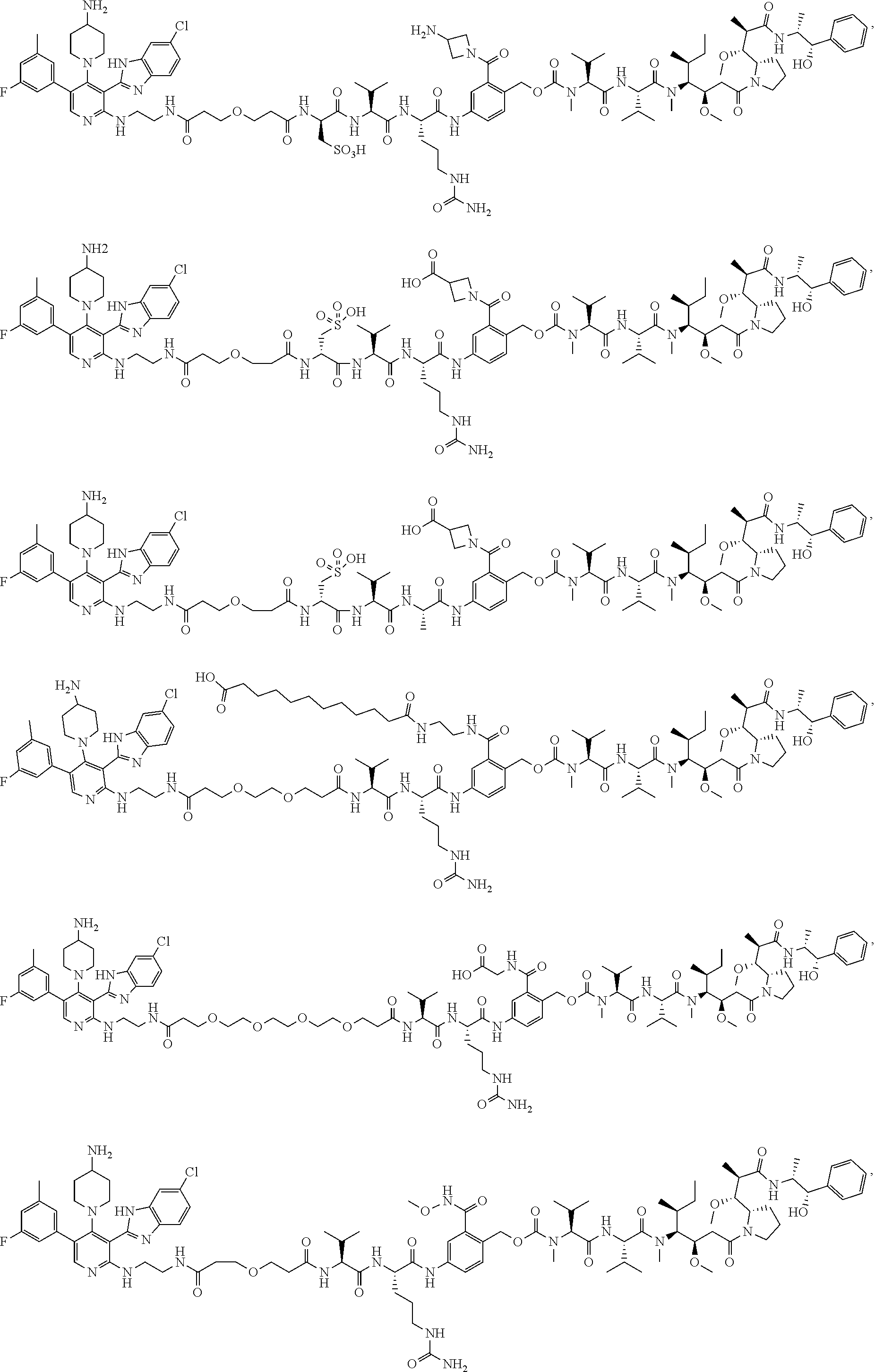

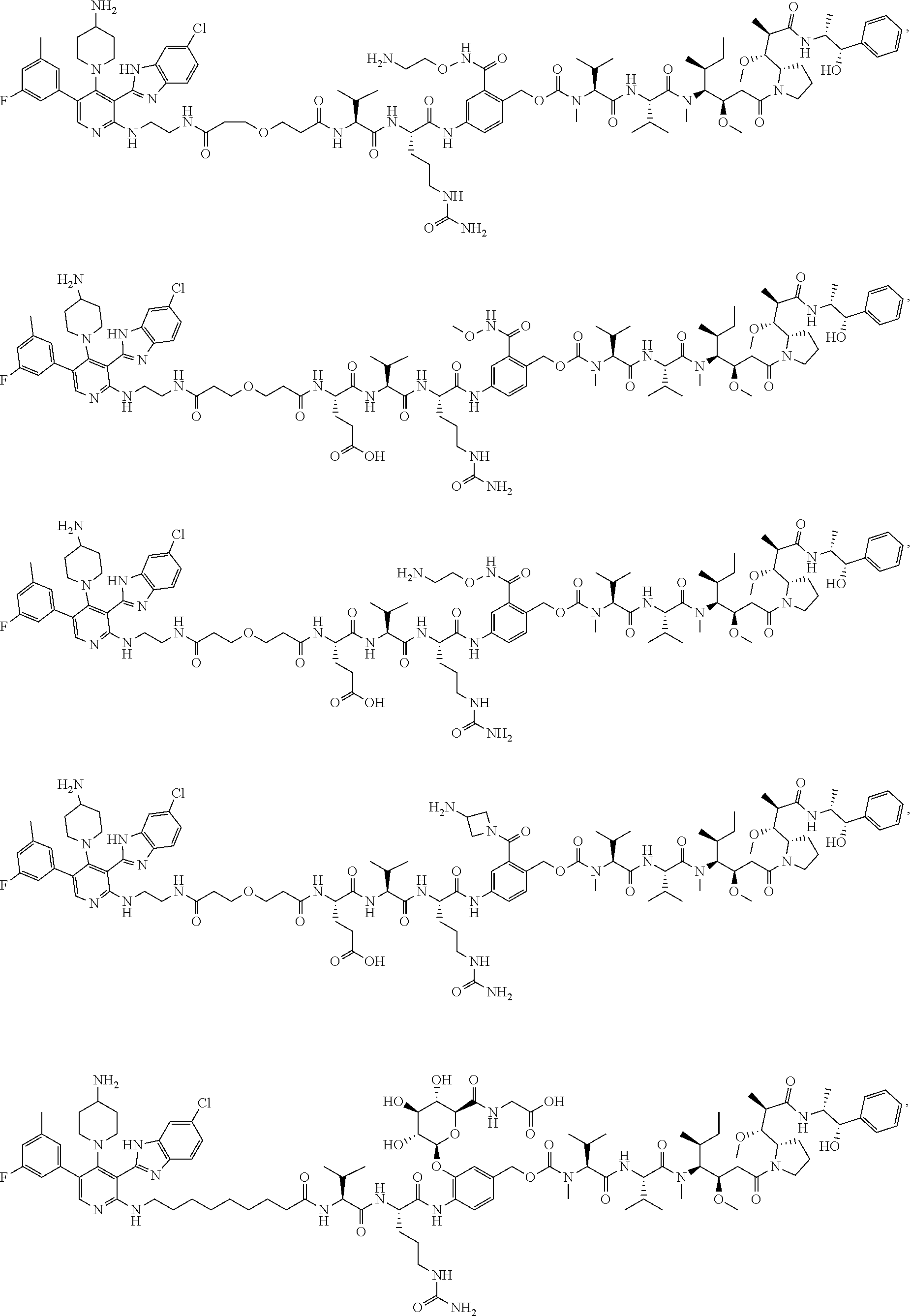

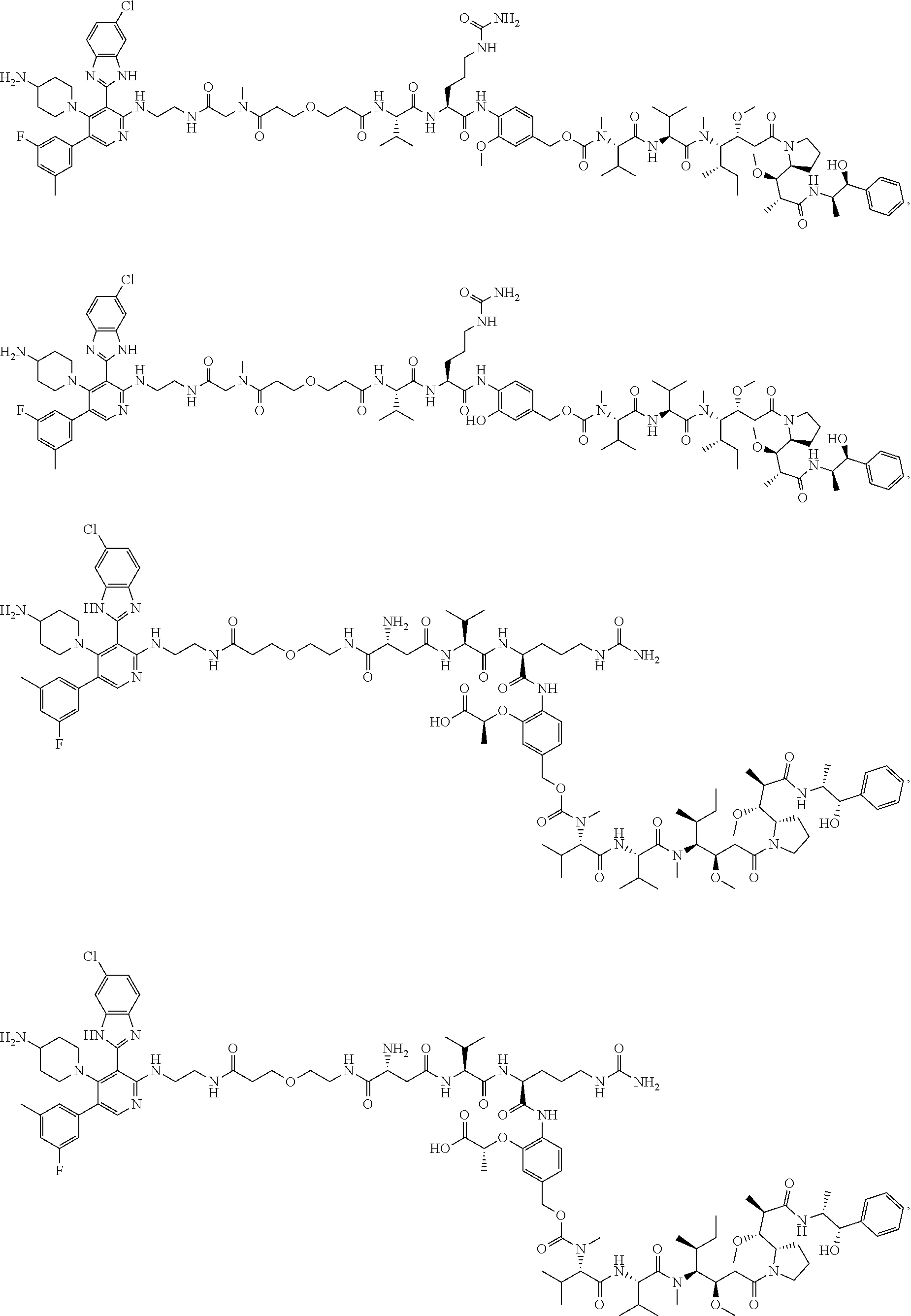

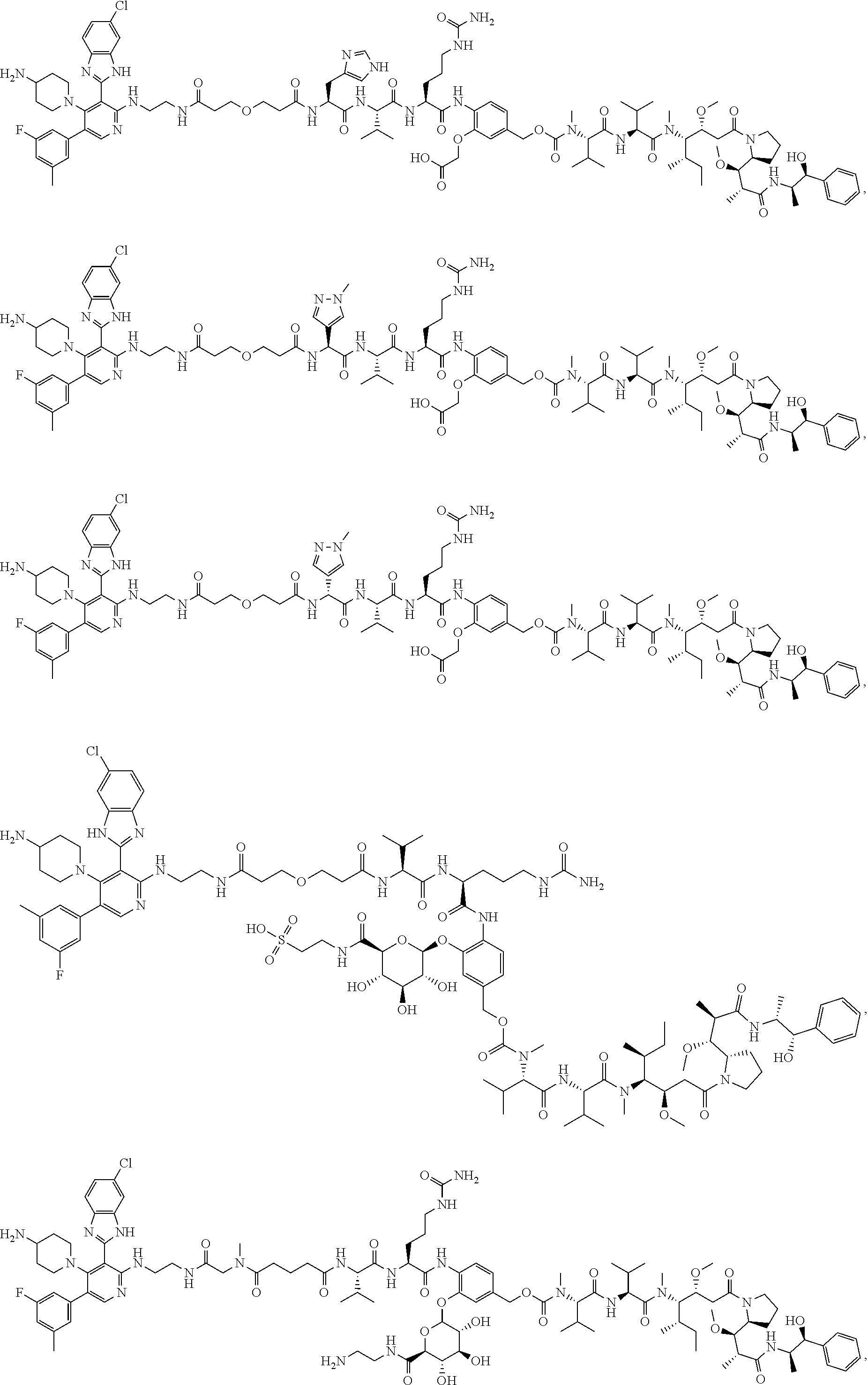

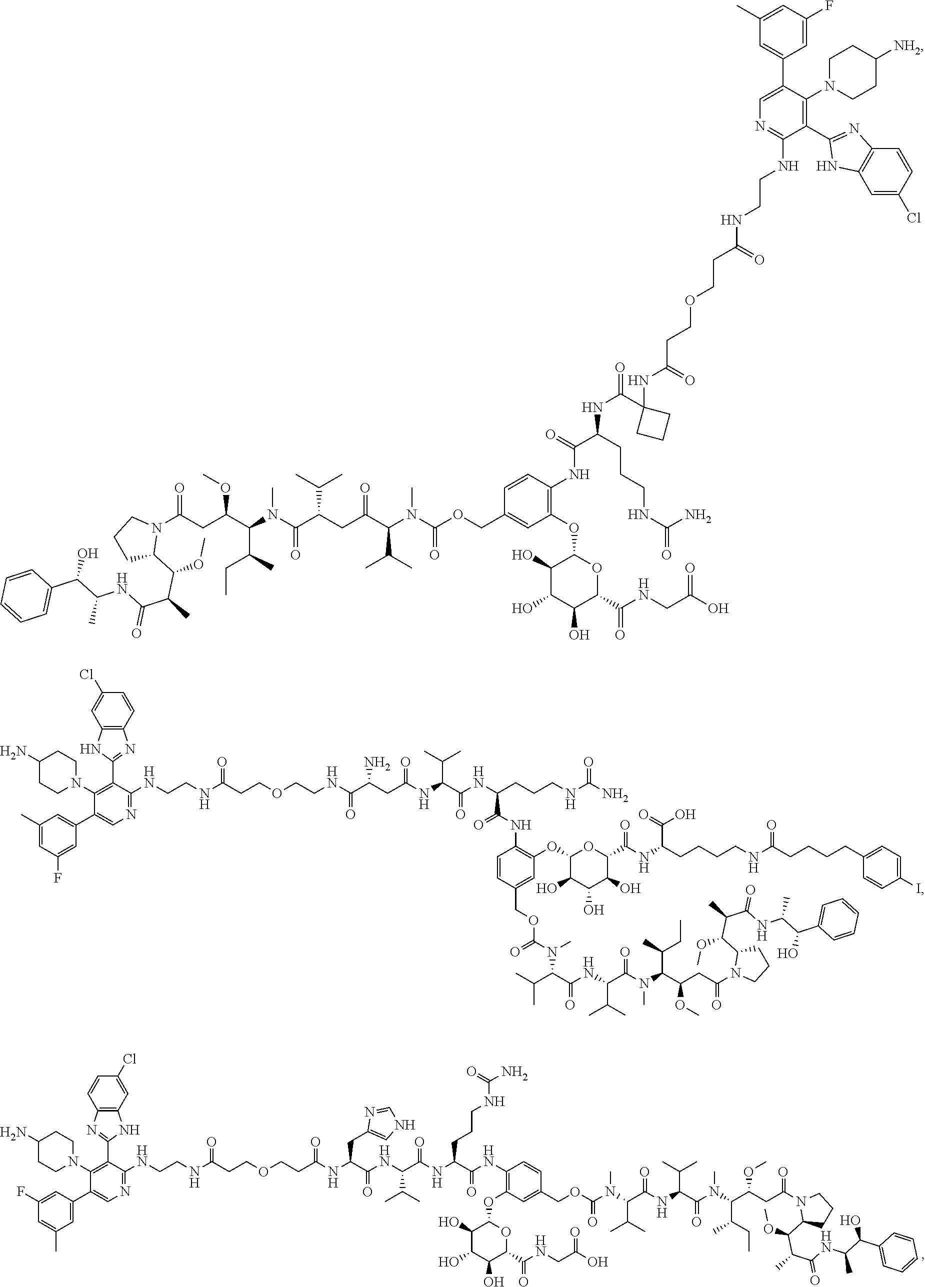

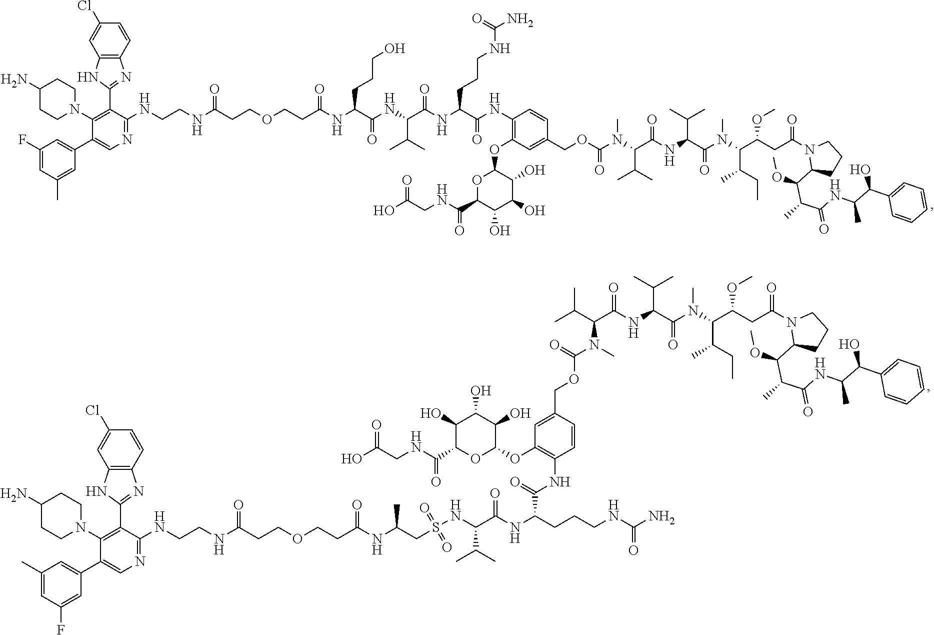

or a pharmaceutically acceptable salt thereof.

In some embodiments, the compound of Formula (I) is Compound 205.

In some embodiments, the compound of Formula (I) is Compound 206.

In some embodiments, the compound of Formula (I) is Compound 207.

In some embodiments, the compound of Formula (I) is Compound 215.

In some embodiments, the compound of Formula (I) is Compound 216.

In some embodiments, the compound of Formula (I) is Compound 217.

In some embodiments, the compound of Formula (I) is Compound 218.

In some embodiments, the compound of Formula (I) is Compound 219.

In some embodiments, the compound of Formula (I) is Compound 220.

In some embodiments, the compound of Formula (I) is Compound 221.

In some embodiments, the compound of Formula (I) is Compound 222.

In some embodiments, the compound of Formula (I) is Compound 223.

In some embodiments, the compound of Formula (I) is Compound 224.

In some embodiments, the compound of Formula (I) is Compound 225.

In some embodiments, the compound of Formula (I) is Compound 226.

In some embodiments, the compound of Formula (I) is Compound 227.

In some embodiments, the compound of Formula (I) is Compound 228.

In some embodiments, the compound of Formula (I) is Compound 229.

In some embodiments, the compound of Formula (I) is Compound 230.

In some embodiments, the compound of Formula (I) is Compound 231.

In some embodiments, the compound of Formula (I) is Compound 232.

In some embodiments, the compound of Formula (I) is Compound 233.

In some embodiments, the compound of Formula (I) is Compound 234.

In some embodiments, the compound of Formula (I) is Compound 235.

In some embodiments, the compound of Formula (I) is Compound 236.

In some embodiments, the compound of Formula (I) is Compound 237.

In some embodiments, the compound of Formula (I) is Compound 238.

In some embodiments, the compound of Formula (I) is Compound 246.

In some embodiments, the compound of Formula (I) is Compound 247.

In some embodiments, the compound of Formula (I) is selected from:

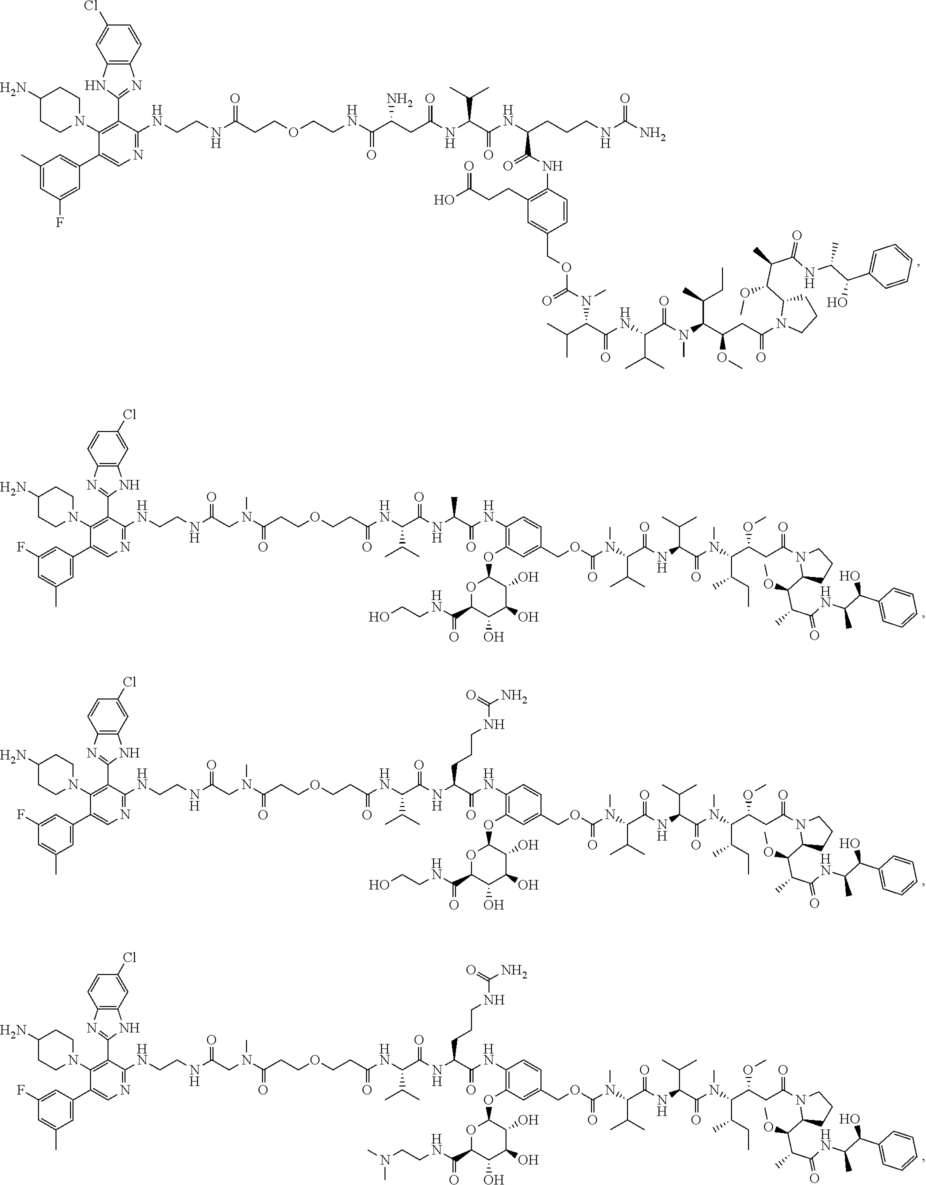

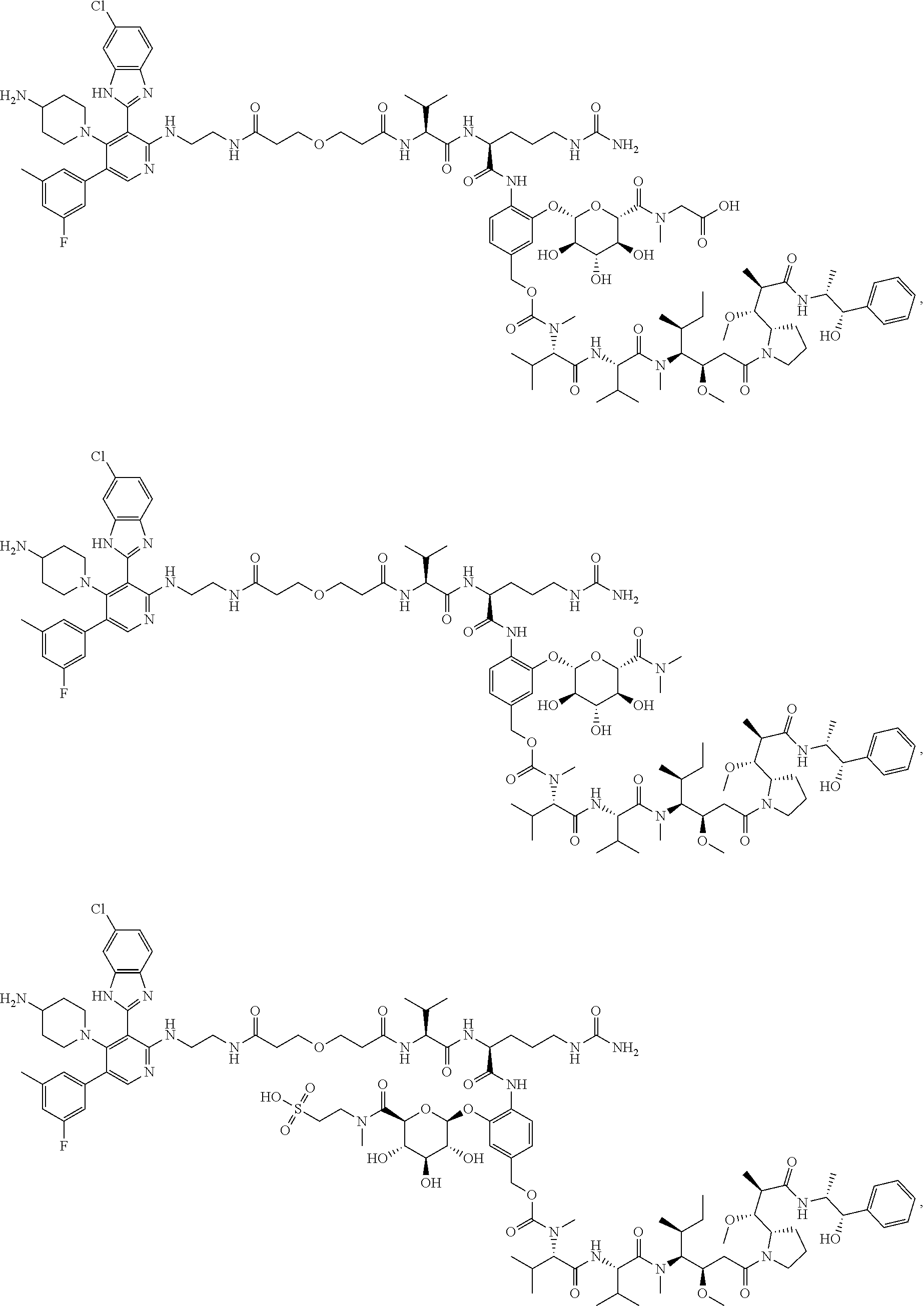

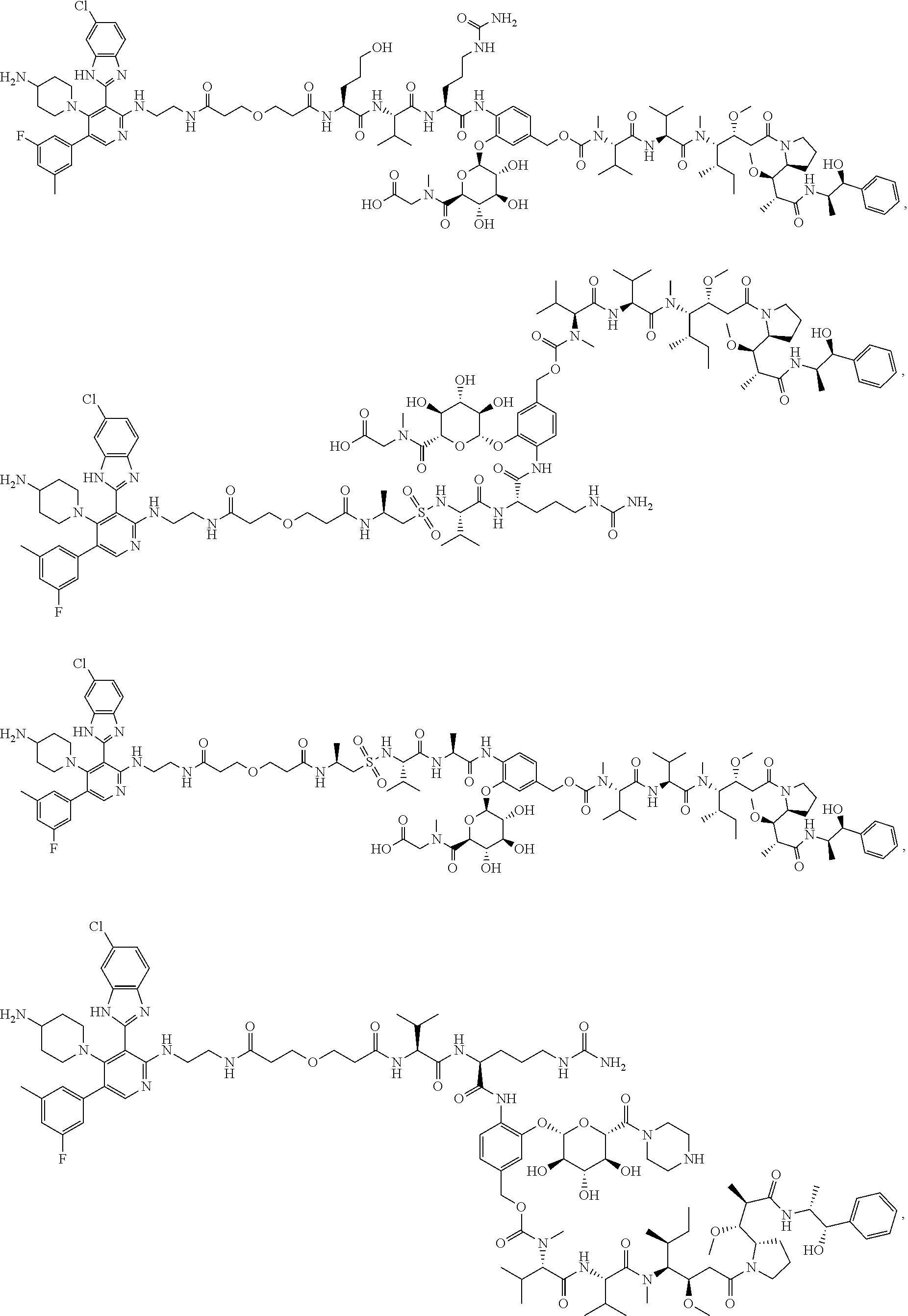

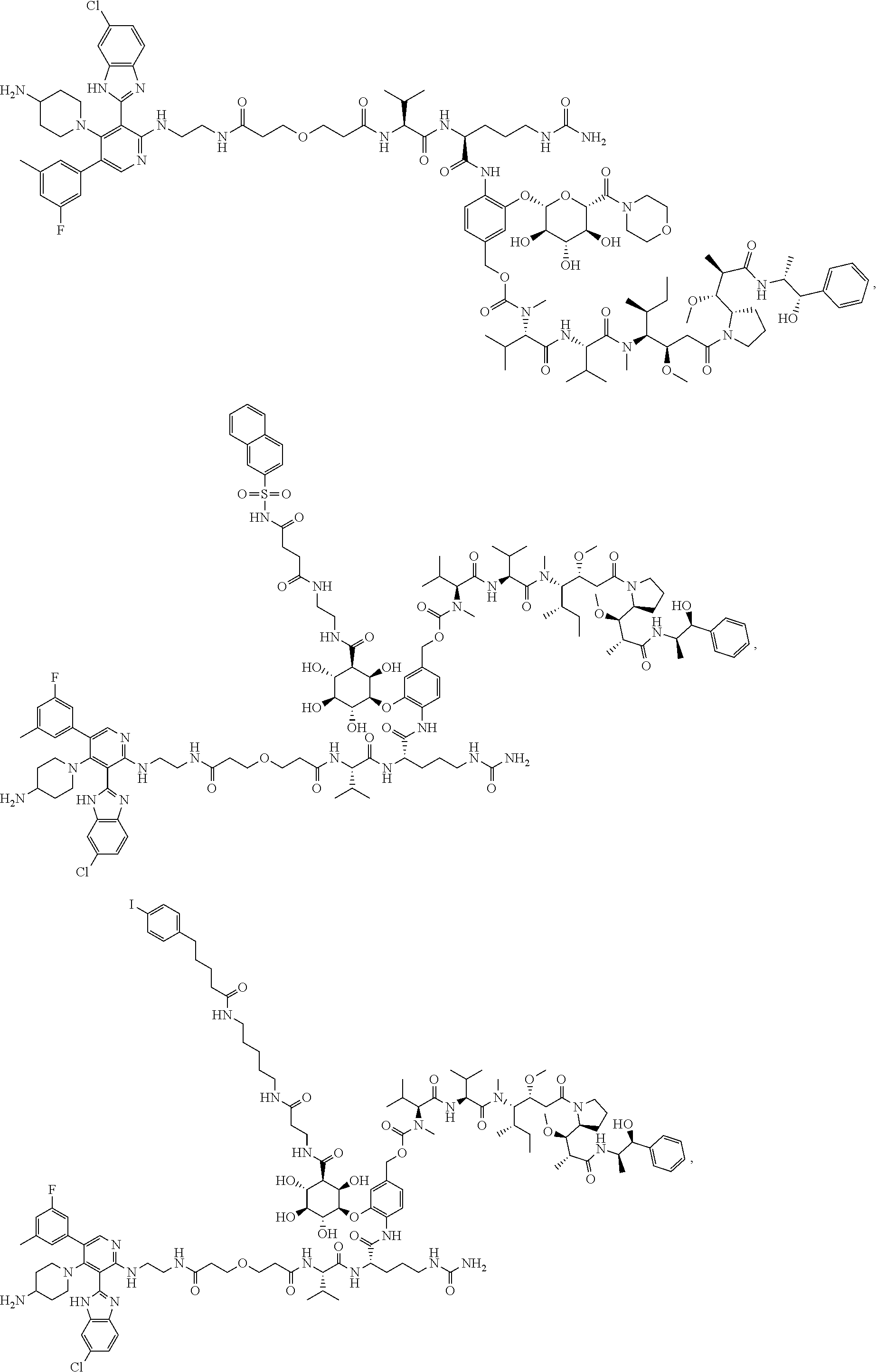

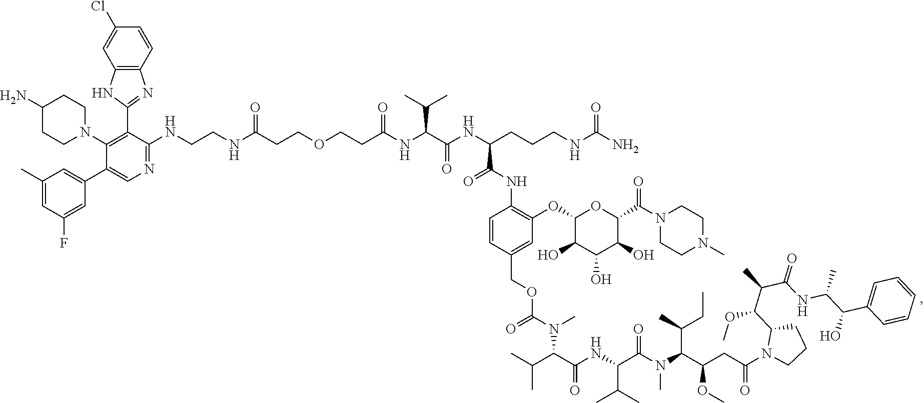 [SMILES: CC[C@H](C)[C@@H]([C@@H](CC(=O)N1CCC[C@H]1[C@H](OC)[C@@H](C)C(=O)N[C@H](C)[C@@H](O)c1ccccc1)OC)N(C)C(=O)[C@@H](NC(=O)[C@H](C(C)C)N(C)C(=O)OCc1ccc(NC(=O)[C@H](CCCNC(N)=O)NC(=O)[C@@H](NC(=O)CCOCCC(=O)NCCNc2ncc(-c3cc(C)cc(F)c3)c(N3CCC(N)CC3)c2-c2nc3ccc(Cl)cc3[nH]2)C(C)C)c(O[C@@H]2O[C@H](C(=O)N3CCN(C)CC3)[C@@H](O)[C@H](O)[C@H]2O)c1)C(C)C]

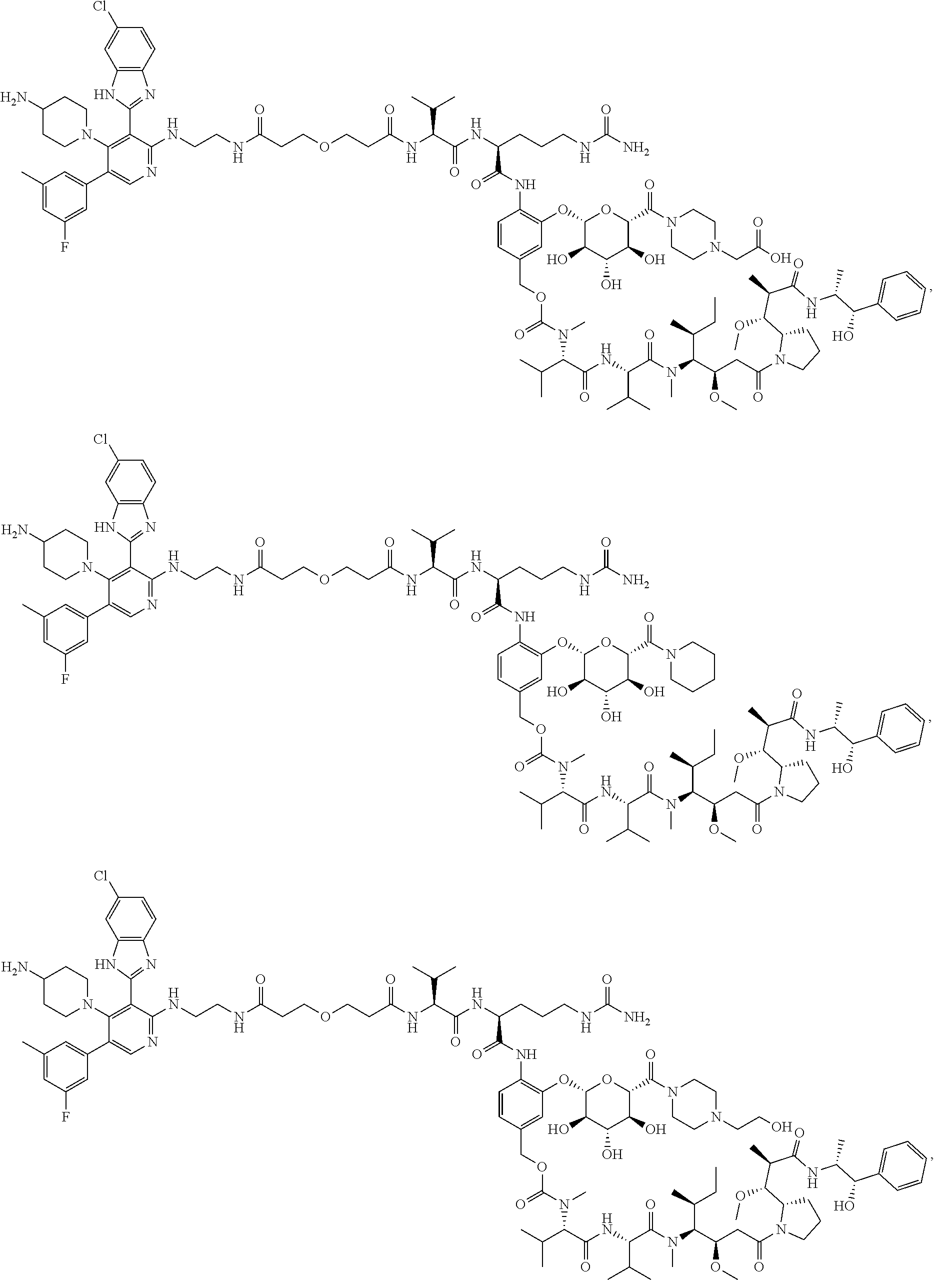

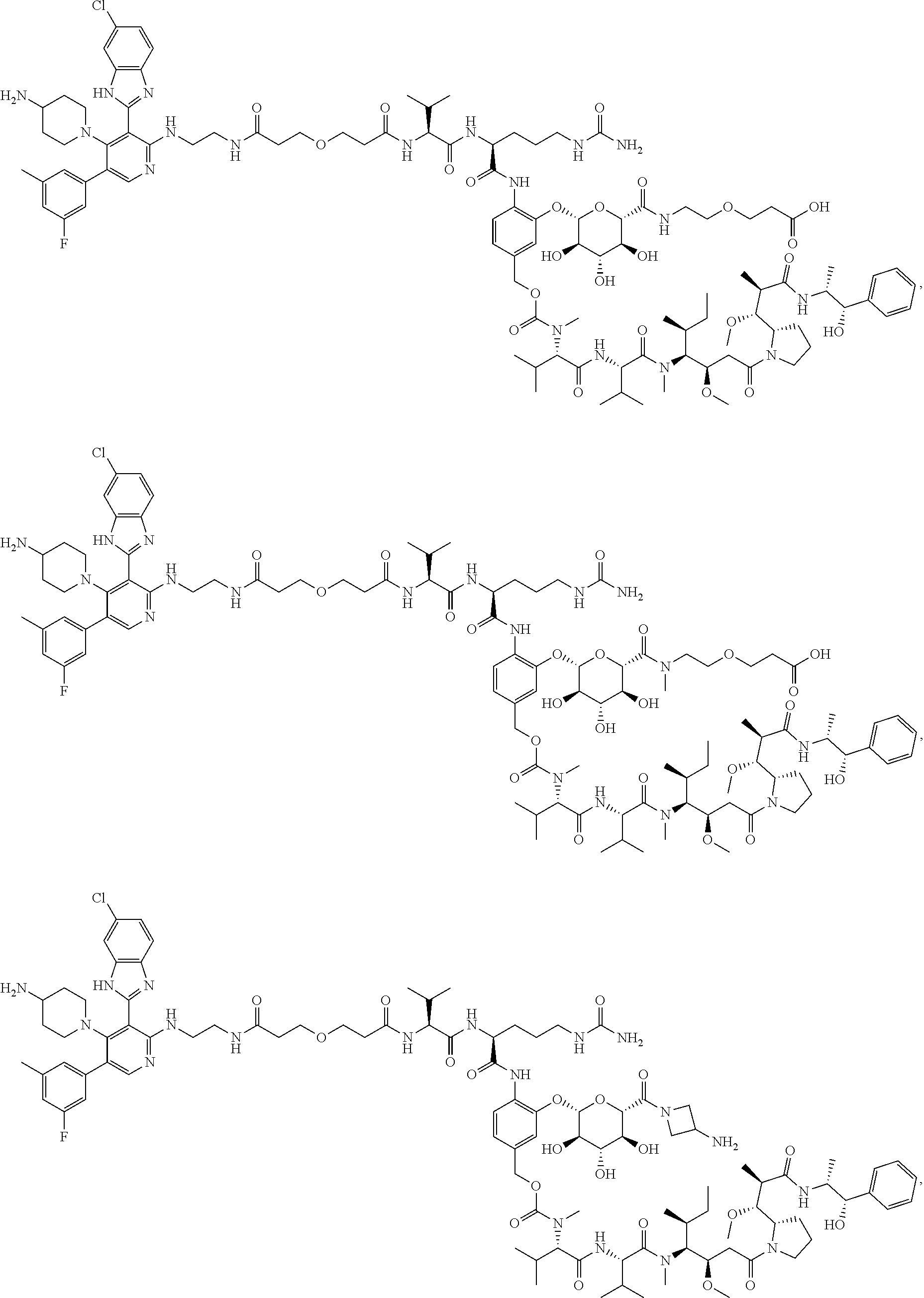

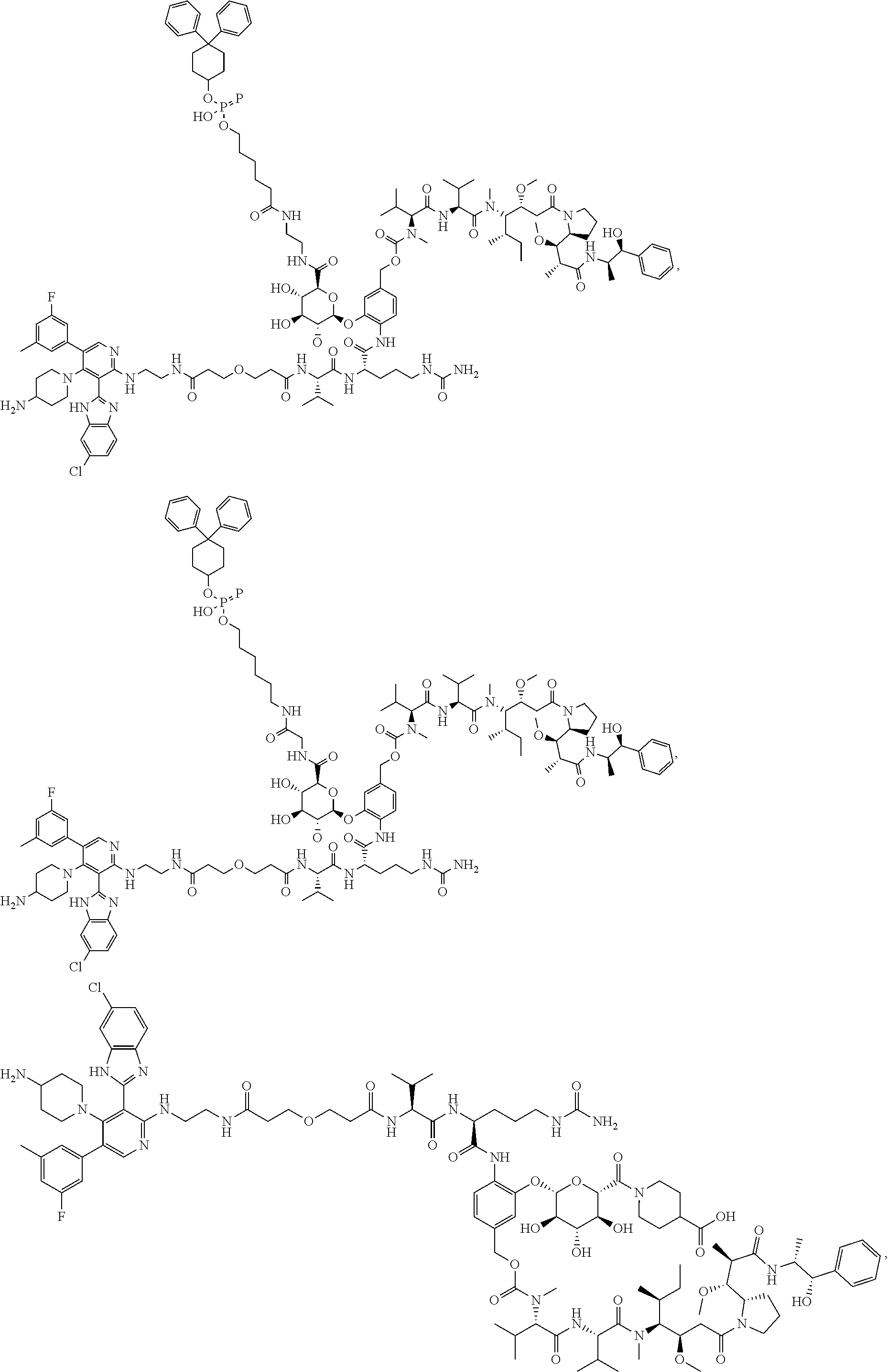

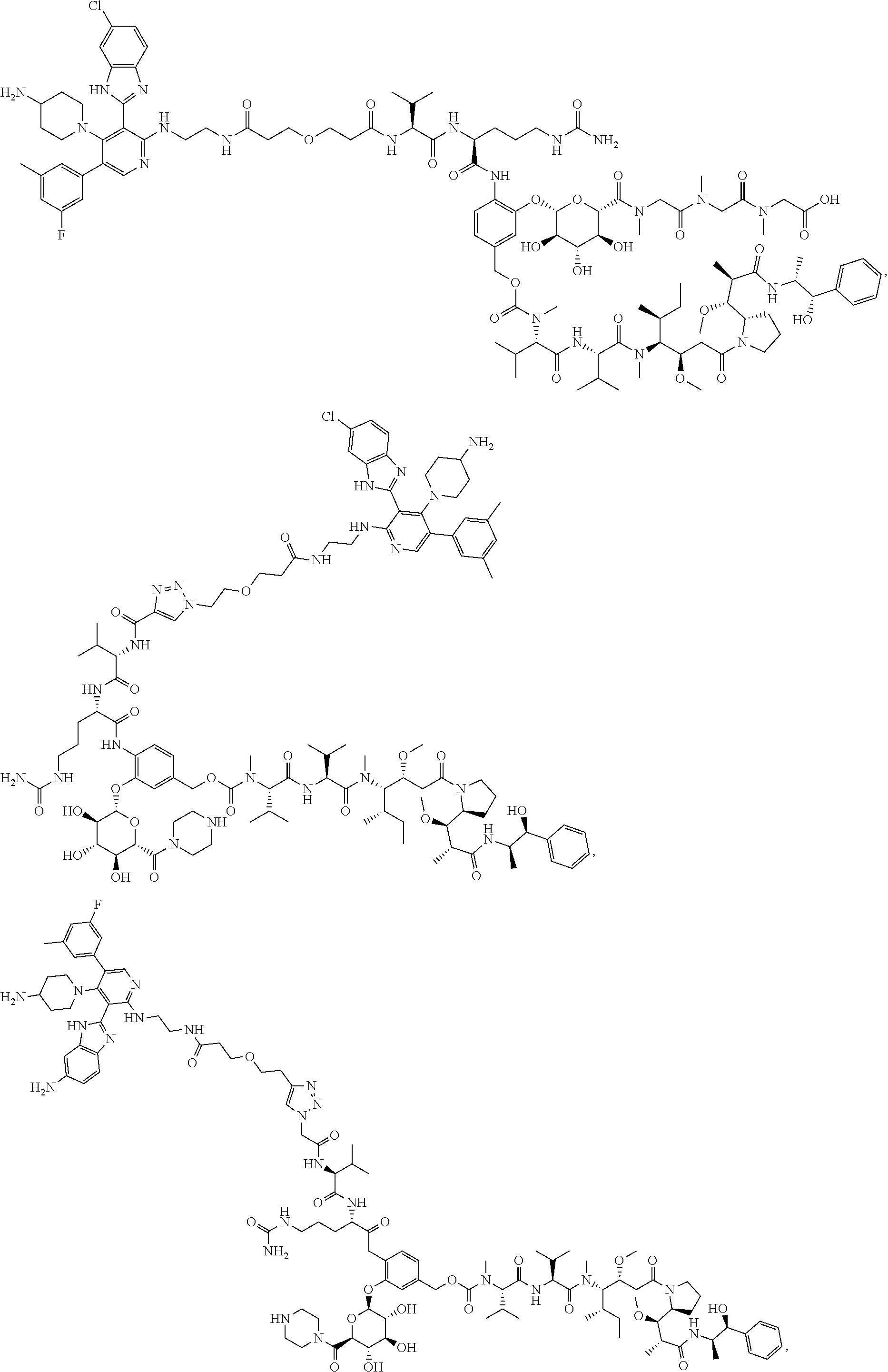

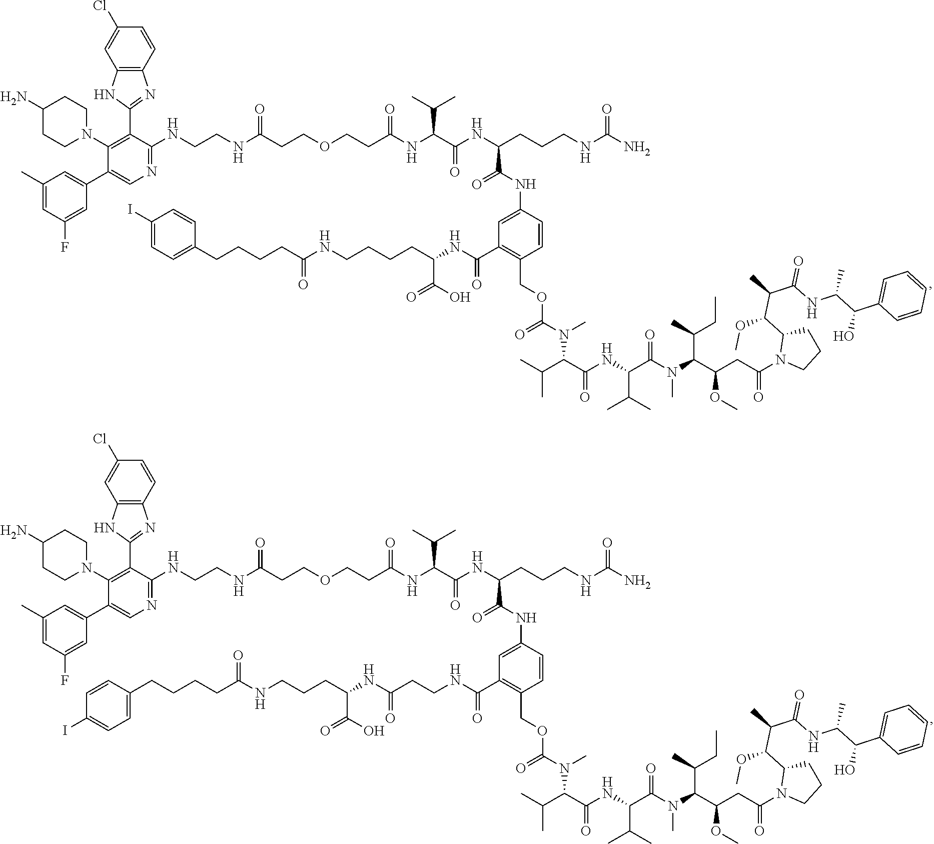

or a pharmaceutically acceptable salt thereof.

### Other Conjugates

In some embodiments, disclosed herein is an NDC.

In some embodiments, the NDC is Compound 57.

In some embodiments, the NDC is Compound 58.

In some embodiments, the NDC is Compound 64.

In some embodiments, the NDC is Compound 66.

In some embodiments, the NDC is Compound 79.

In some embodiments, the NDC is Compound 80.

In some embodiments, the NDC is Compound 81.

In some embodiments, the NDC is Compound 82.

In some embodiments, the NDC is Compound 84.

In some embodiments, the NDC is Compound 85.

In some embodiments, the NDC is Compound 92.

In some embodiments, the NDC is Compound 93.

In some embodiments, the NDC is Compound 94.

In some embodiments, the NDC is Compound 95.

In some embodiments, the NDC is Compound 96.

In some embodiments, the NDC is Compound 97.

In some embodiments, the NDC is Compound 98.

In some embodiments, the NDC is Compound 99.

In some embodiments, the NDC is Compound 100.

In some embodiments, the NDC is Compound 101.

In some embodiments, the NDC is Compound 102.

In some embodiments, the NDC is Compound 103.

In some embodiments, the NDC is Compound 104.

In some embodiments, the NDC is Compound 105.

In some embodiments, the NDC is Compound 106.

In some embodiments, the NDC is Compound 107.

In some embodiments, the NDC is Compound 108.

In some embodiments, the NDC is Compound 109.

In some embodiments, the NDC is Compound 110.

In some embodiments, the NDC is Compound 111.

In some embodiments, the NDC is Compound 112.

In some embodiments, the NDC is Compound 113.

In some embodiments, the NDC is Compound 114.

In some embodiments, the NDC is Compound 115.

In some embodiments, the NDC is Compound 116.

In some embodiments, the NDC is Compound 117.

In some embodiments, the NDC is Compound 118.

In some embodiments, the NDC is Compound 119.

In some embodiments, the NDC is Compound 120.

In some embodiments, the NDC is Compound 121.

In some embodiments, the NDC is Compound 122.

In some embodiments, the NDC is Compound 123.

In some embodiments, the NDC is Compound 124.

In some embodiments, the NDC is Compound 125.

In some embodiments, the NDC is Compound 126.

In some embodiments, the NDC is Compound 127.

In some embodiments, the NDC is Compound 128.

In some embodiments, the NDC is Compound 129.

In some embodiments, the NDC is Compound 130.

In some embodiments, the NDC is Compound 131.

In some embodiments, the NDC is Compound 132.

In some embodiments, the NDC is Compound 133.

In some embodiments, the NDC is Compound 134.

In some embodiments, the NDC is Compound 135.

In some embodiments, the NDC is Compound 136.

In some embodiments, the NDC is Compound 137.

In some embodiments, the NDC is Compound 138.

In some embodiments, the NDC is Compound 139.

In some embodiments, the NDC is Compound 140.

In some embodiments, the NDC is Compound 141.

In some embodiments, the NDC is Compound 142.

In some embodiments, the NDC is Compound 143.

In some embodiments, the NDC is Compound 144.

In some embodiments, the NDC is Compound 145.

In some embodiments, the NDC is Compound 146.

In some embodiments, the NDC is Compound 147.

In some embodiments, the NDC is Compound 148.

In some embodiments, the NDC is Compound 149.

In some embodiments, the NDC is Compound 150.

In some embodiments, the NDC is Compound 151.

In some embodiments, the NDC is Compound 152.

In some embodiments, the NDC is Compound 153.

In some embodiments, the NDC is Compound 154.

In some embodiments, the NDC is Compound 155.

In some embodiments, the NDC is Compound 156.

In some embodiments, the NDC is Compound 157.

In some embodiments, the NDC is Compound 158.

In some embodiments, the NDC is Compound 159.

In some embodiments, the NDC is Compound 160.

In some embodiments, the NDC is Compound 161.

In some embodiments, the NDC is Compound 162.

In some embodiments, the NDC is Compound 163.

In some embodiments, the NDC is Compound 164.

In some embodiments, the NDC is Compound 165.

In some embodiments, the NDC is Compound 166.

In some embodiments, the NDC is Compound 167.

In some embodiments, the NDC is Compound 168.

In some embodiments, the NDC is Compound 169.

In some embodiments, the NDC is Compound 170.

In some embodiments, the NDC is Compound 171.

In some embodiments, the NDC is Compound 172.

In some embodiments, the NDC is Compound 173.

In some embodiments, the NDC is Compound 174.

In some embodiments, the NDC is Compound 175.

In some embodiments, the NDC is Compound 176.

In some embodiments, the NDC is Compound 177.

In some embodiments, the NDC is Compound 178.

In some embodiments, the NDC is Compound 179.

In some embodiments, the NDC is Compound 180.

In some embodiments, the NDC is Compound 181.

In some embodiments, the NDC is Compound 182.

In some embodiments, the NDC is Compound 183.

In some embodiments, the NDC is Compound 184.

In some embodiments, the NDC is Compound 185.

In some embodiments, the NDC is Compound 186.

In some embodiments, the NDC is Compound 187.

In some embodiments, the NDC is Compound 188.

In some embodiments, the NDC is Compound 189.

In some embodiments, the NDC is Compound 190.

In some embodiments, the NDC is Compound 191.

In some embodiments, the NDC is Compound 192.

In some embodiments, the NDC is Compound 193.

In some embodiments, the NDC is Compound 194.

In some embodiments, the NDC is Compound 195.

In some embodiments, the NDC is Compound 196.

In some embodiments, the NDC is Compound 197.

In some embodiments, the NDC is Compound 239.

In some embodiments, the NDC is Compound 240.

In some embodiments, the NDC is Compound 241.

In some embodiments, the NDC is Compound 242.

In some embodiments, the NDC is Compound 243.

In some embodiments, the NDC is Compound 244.

In some embodiments, the NDC is Compound 245.

In some embodiments, the NDC is Compound 248.

In some embodiments, the NDC is Compound 249.

In some embodiments, the NDC is selected from:

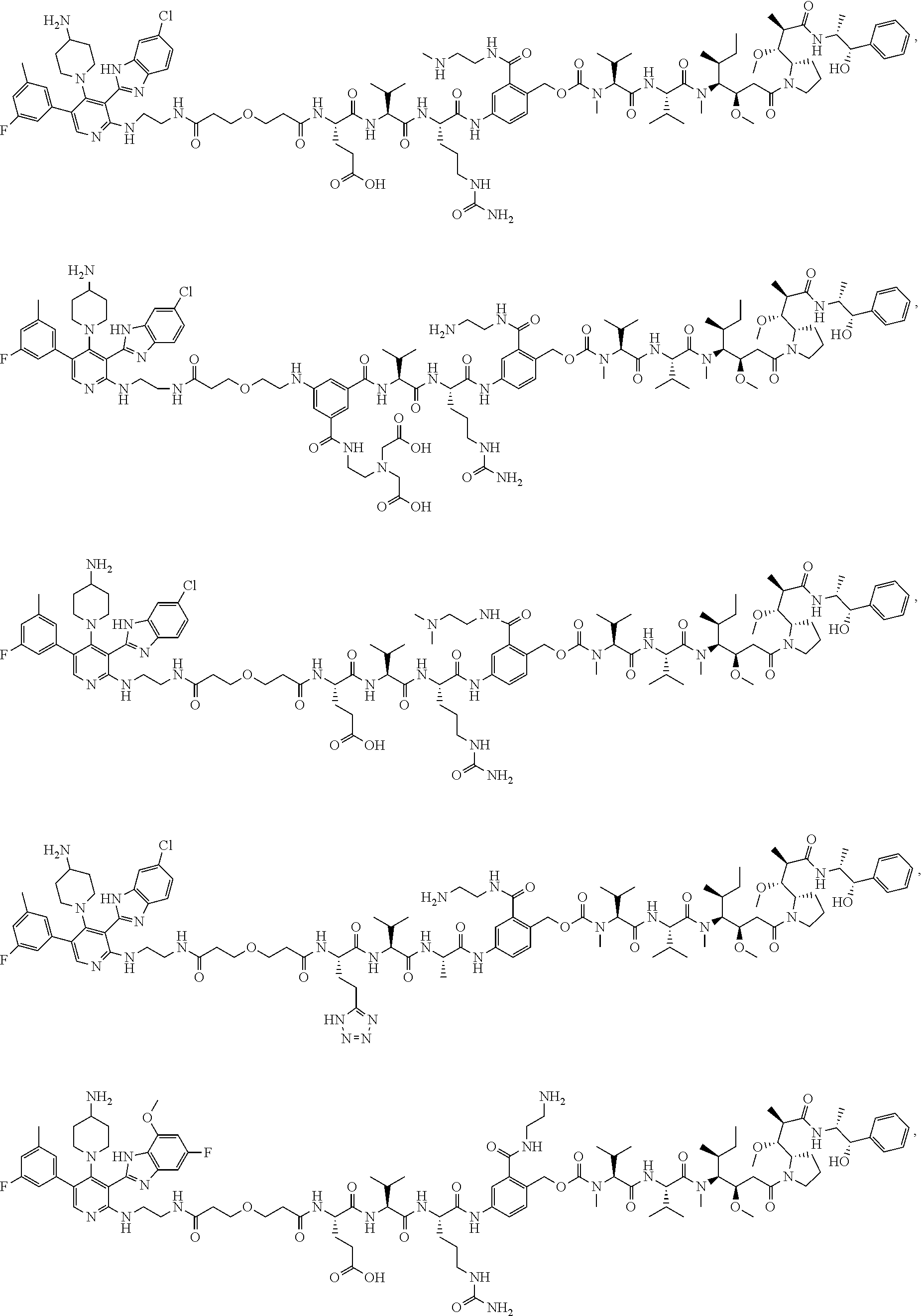

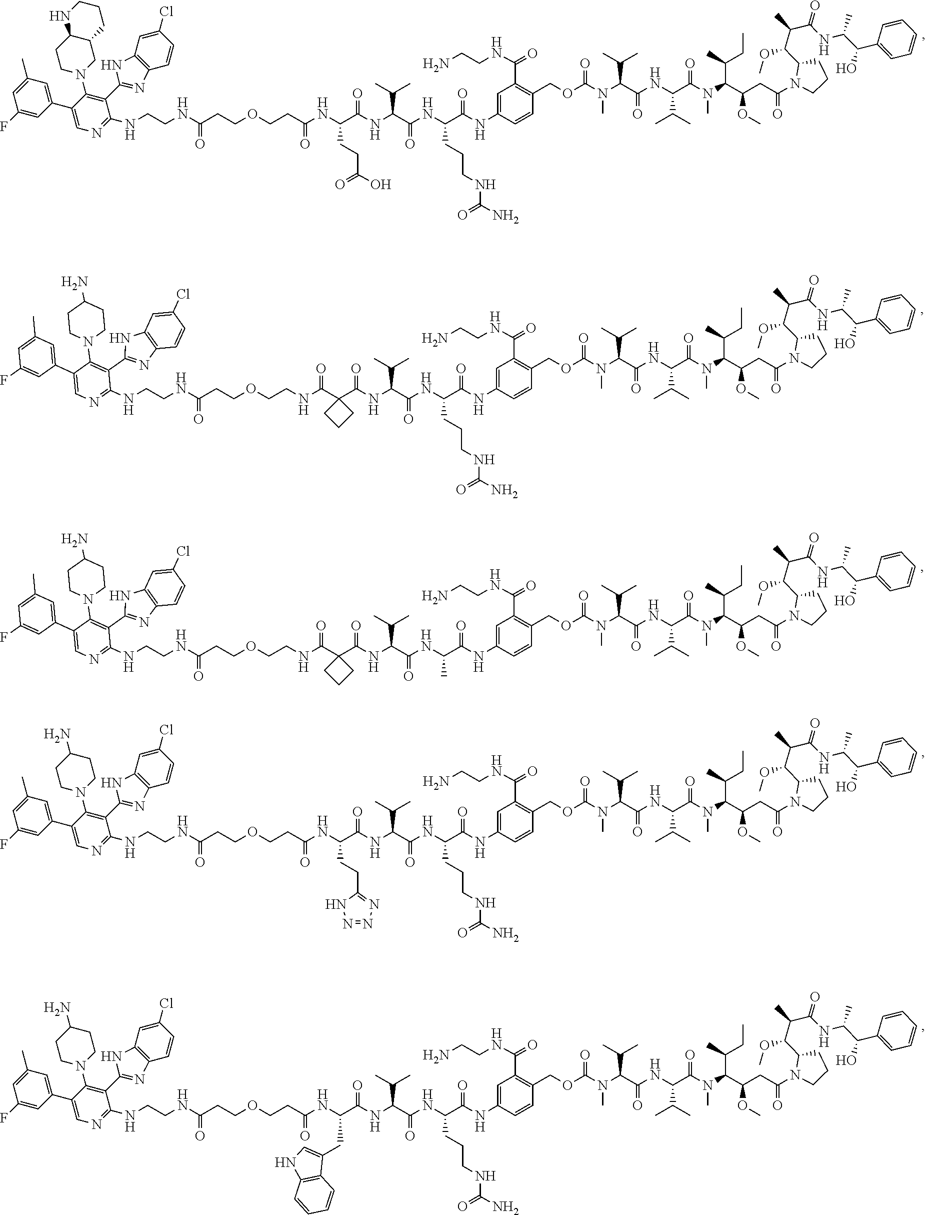

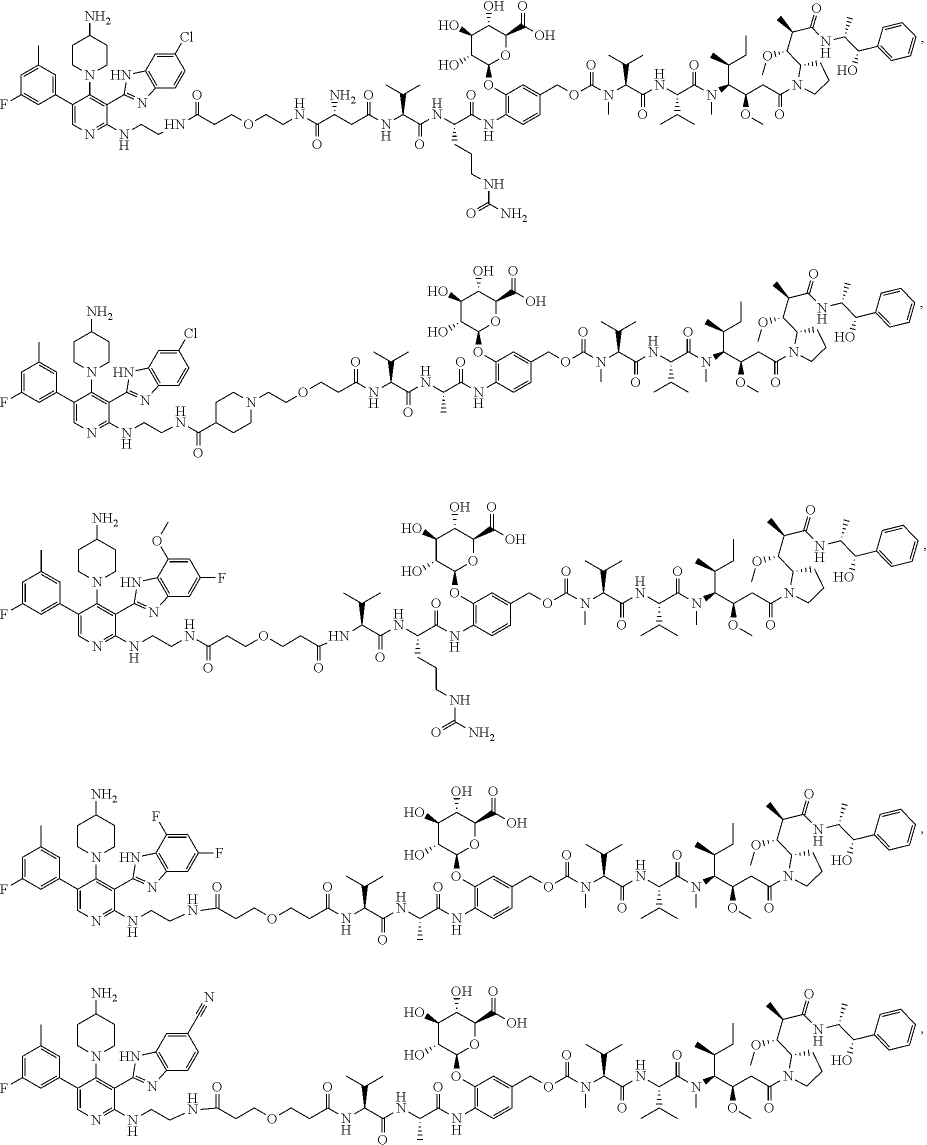

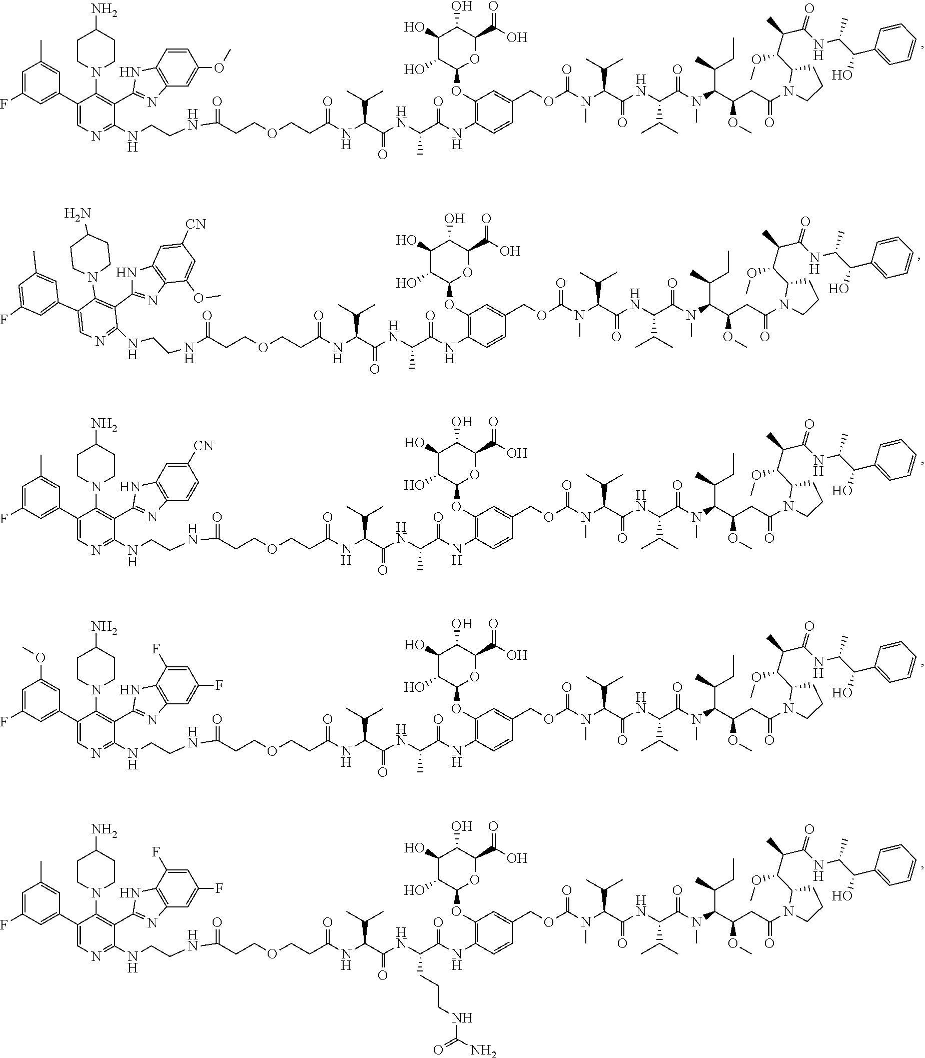

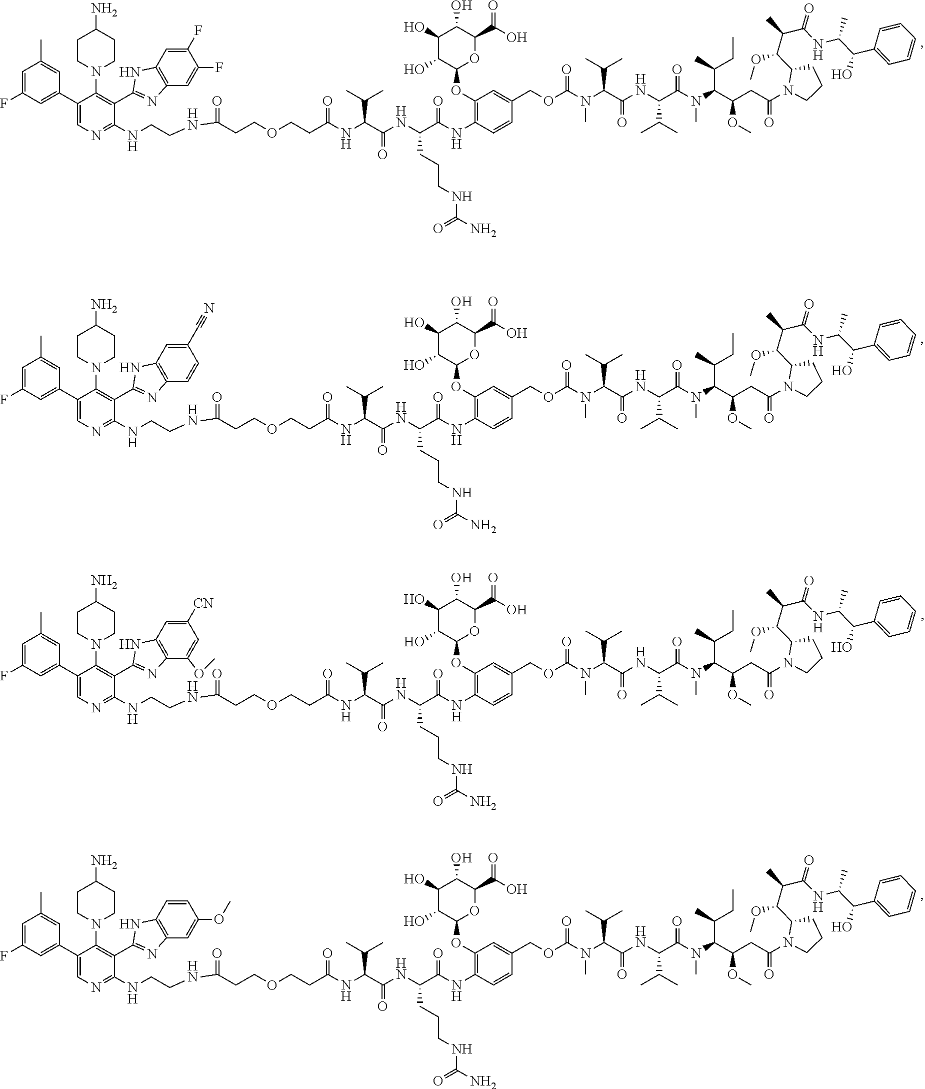

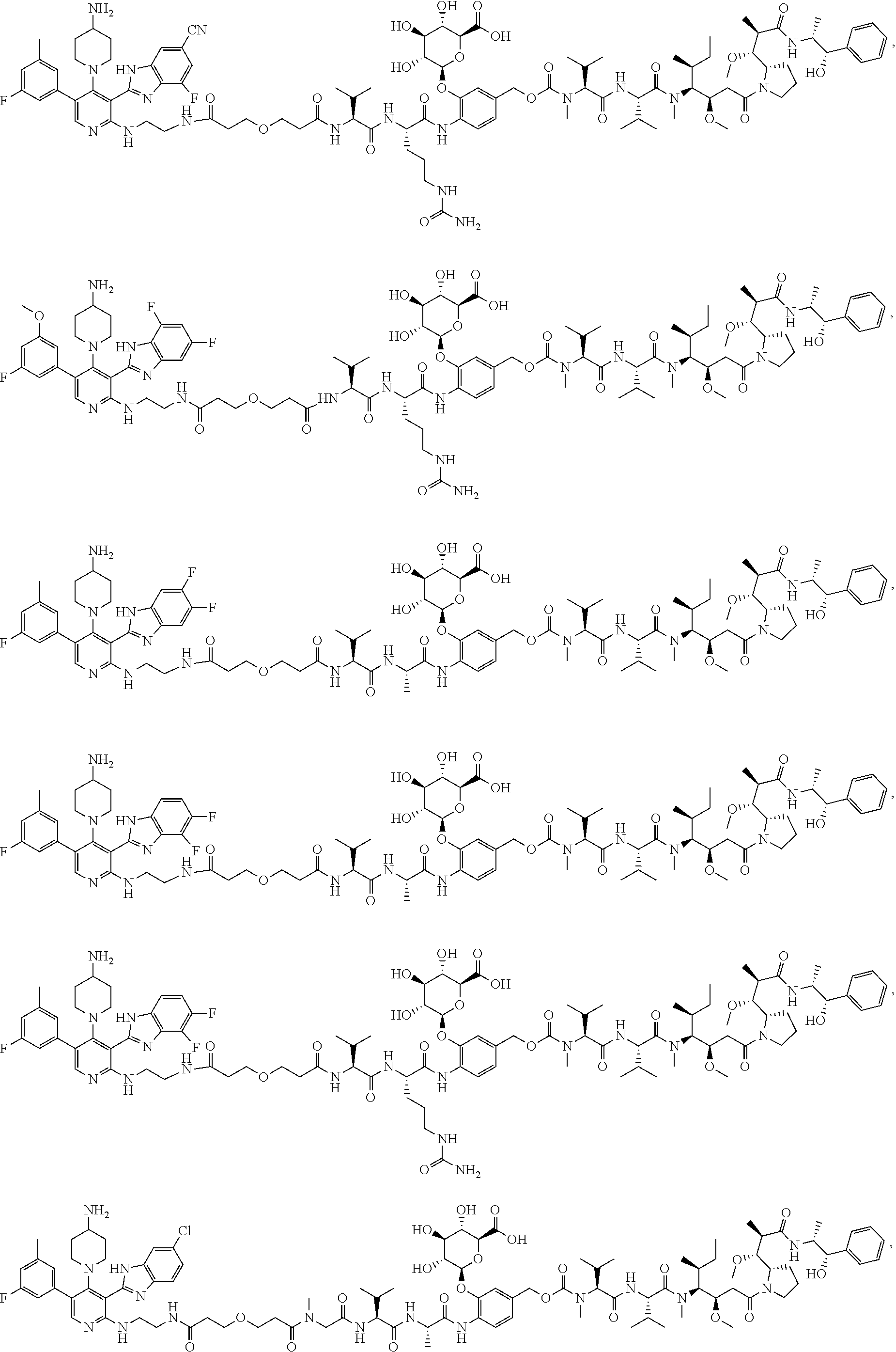

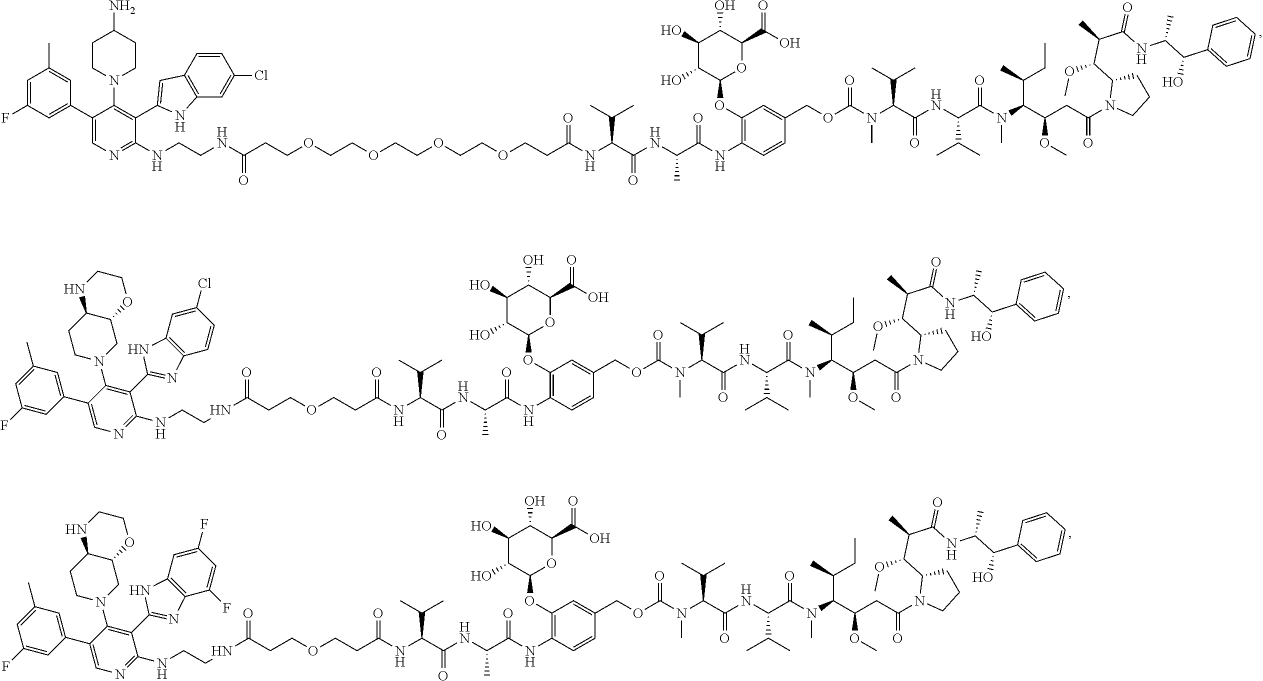

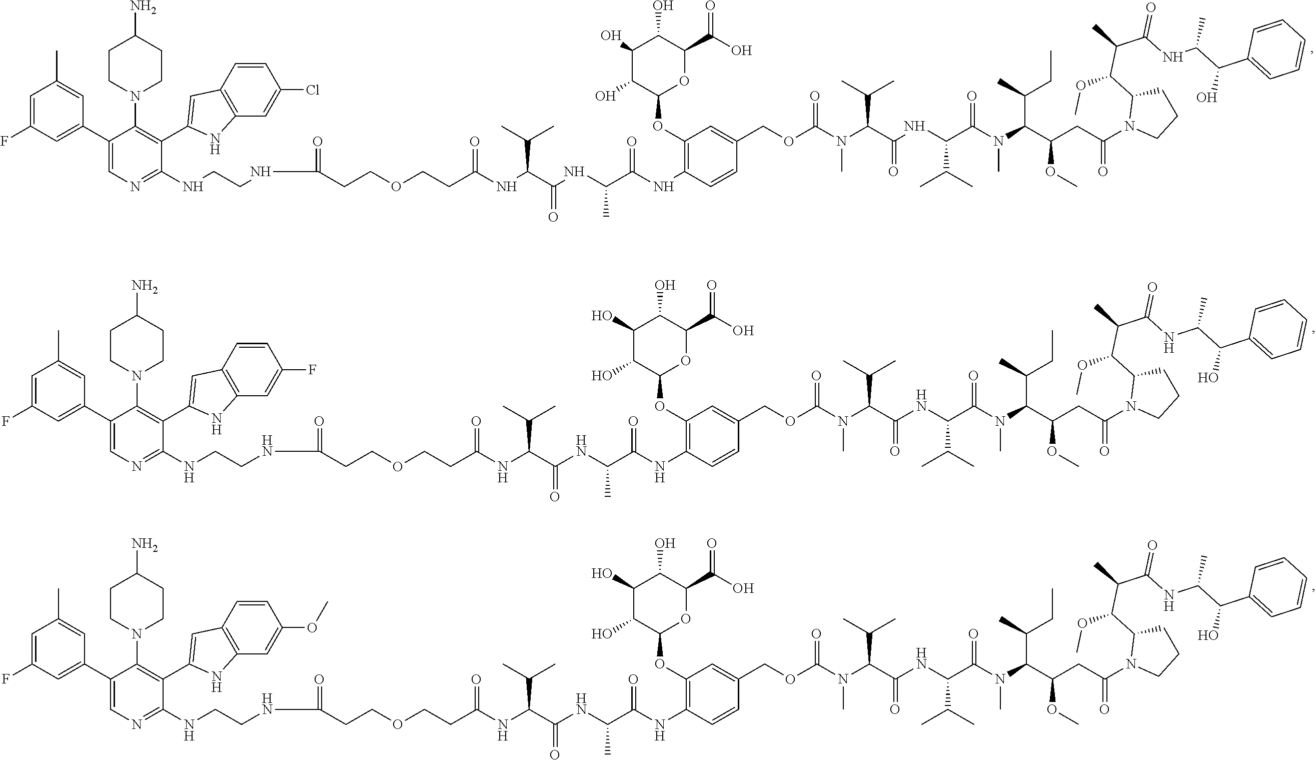

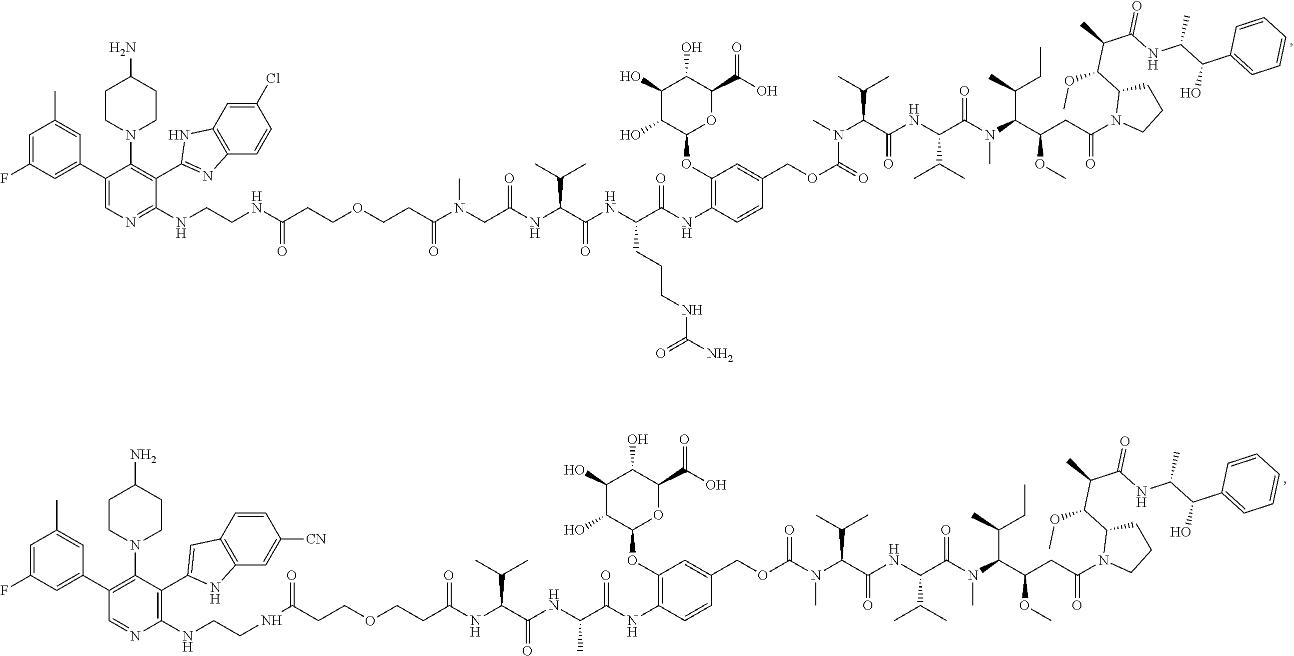

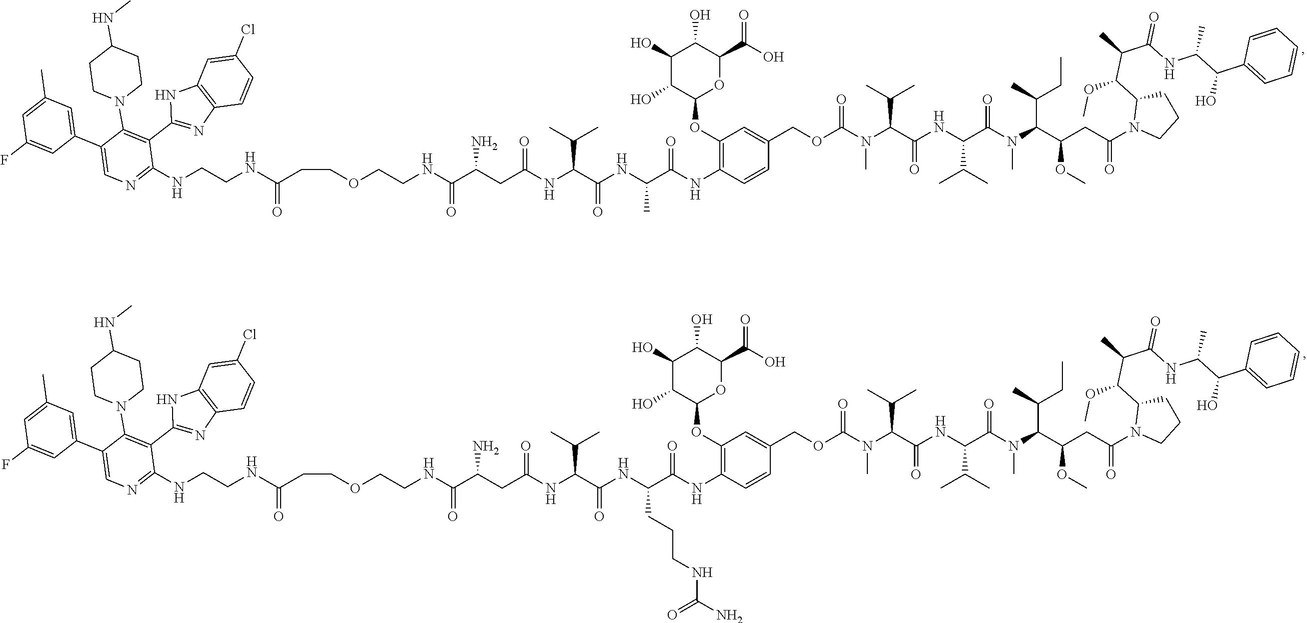

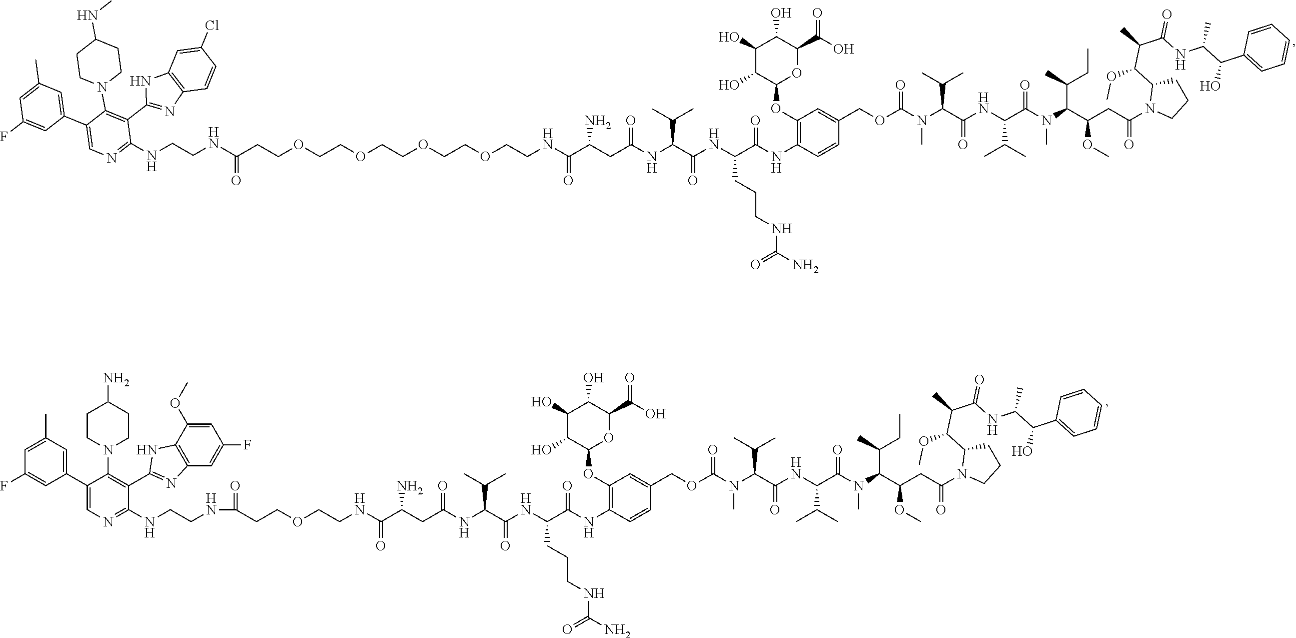

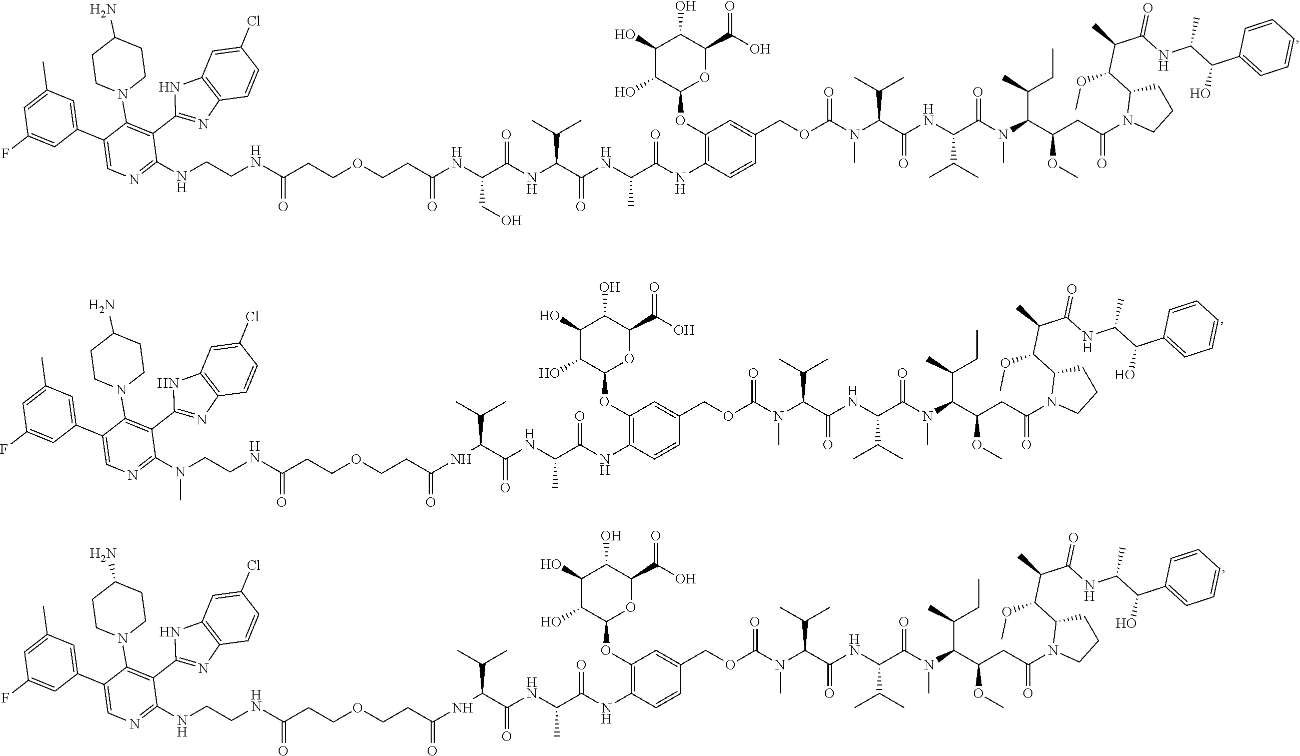

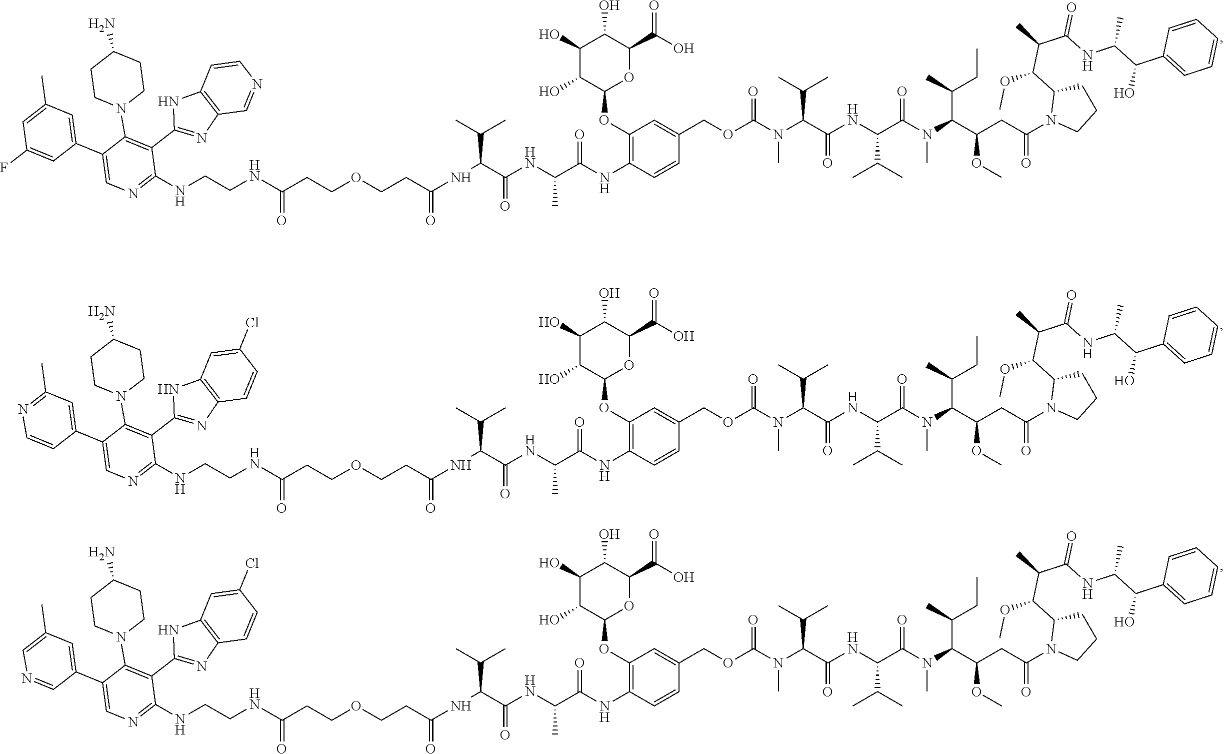

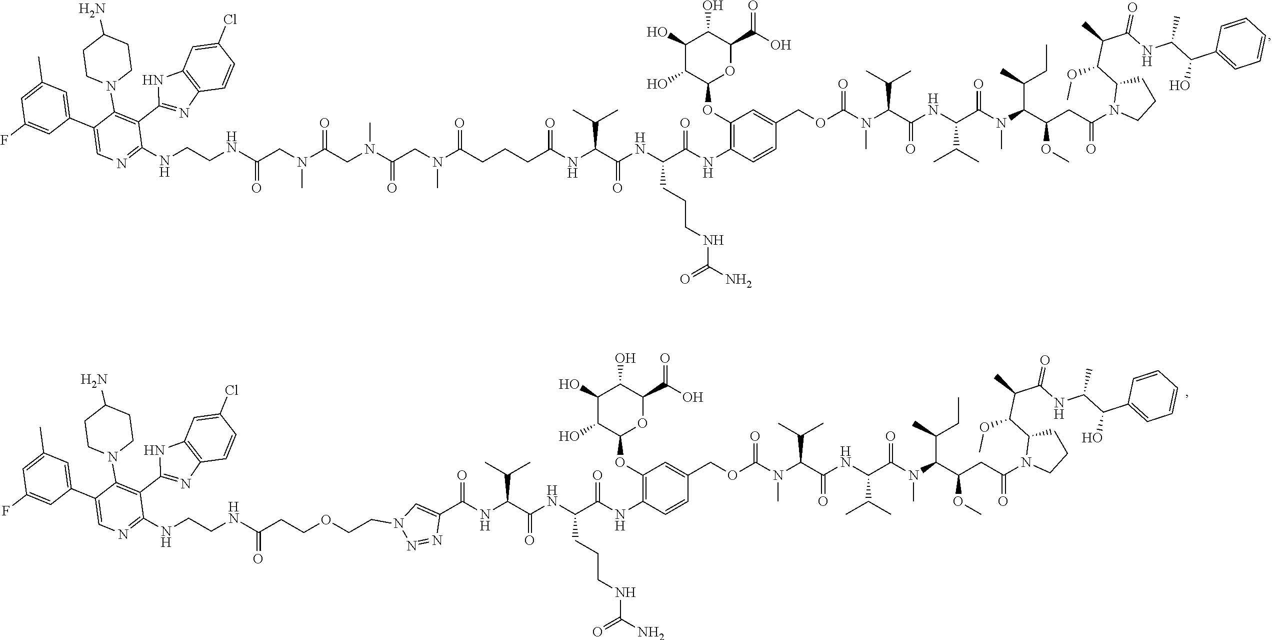

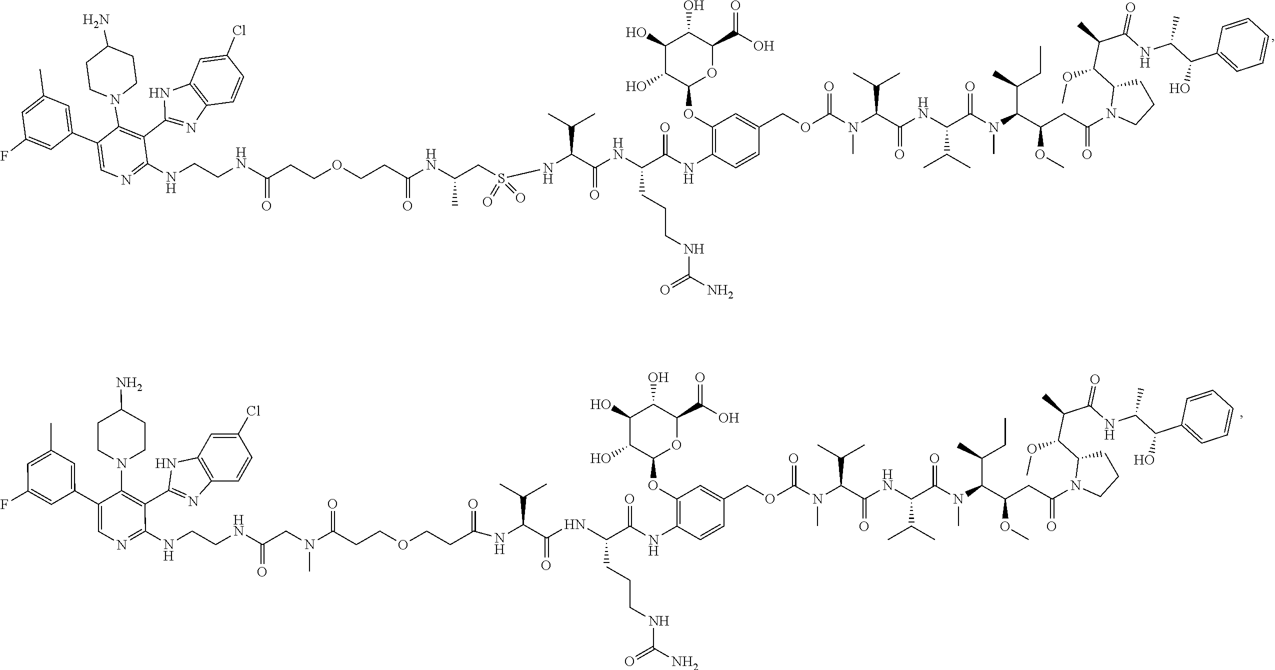

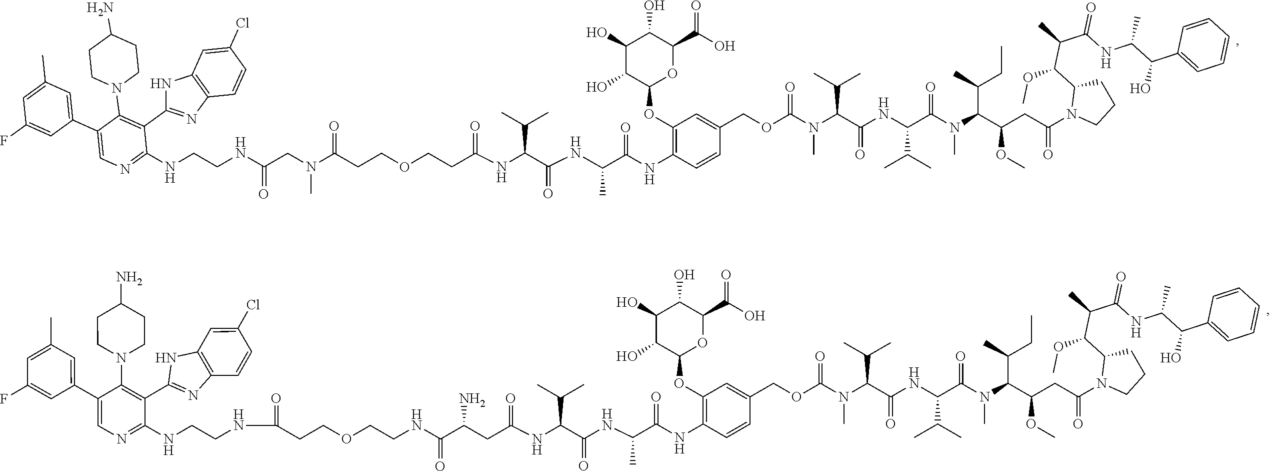

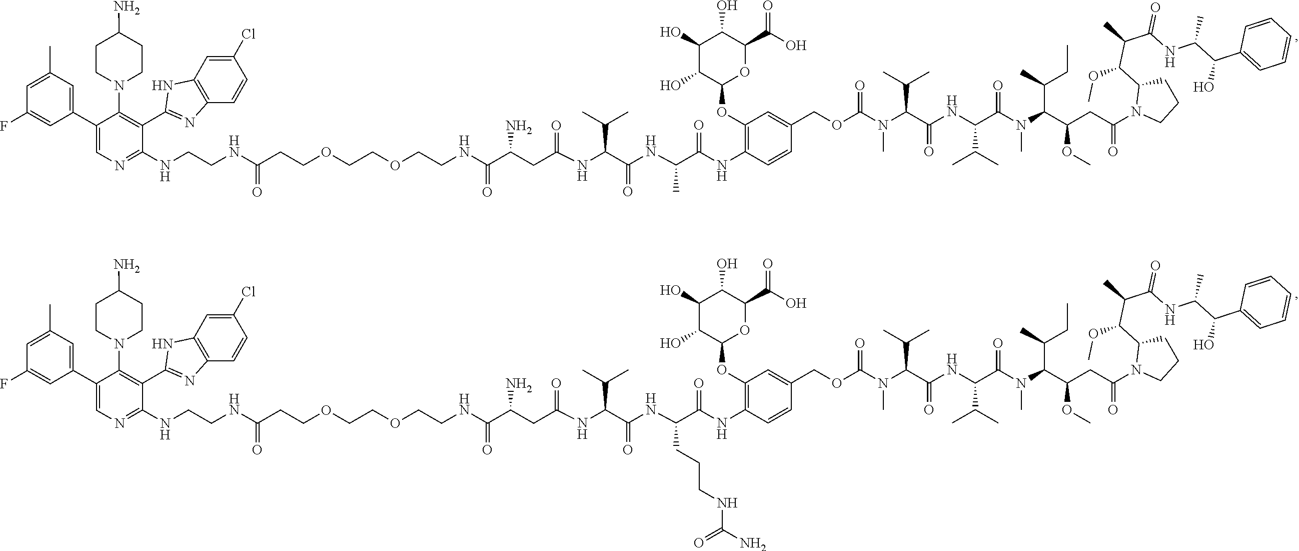

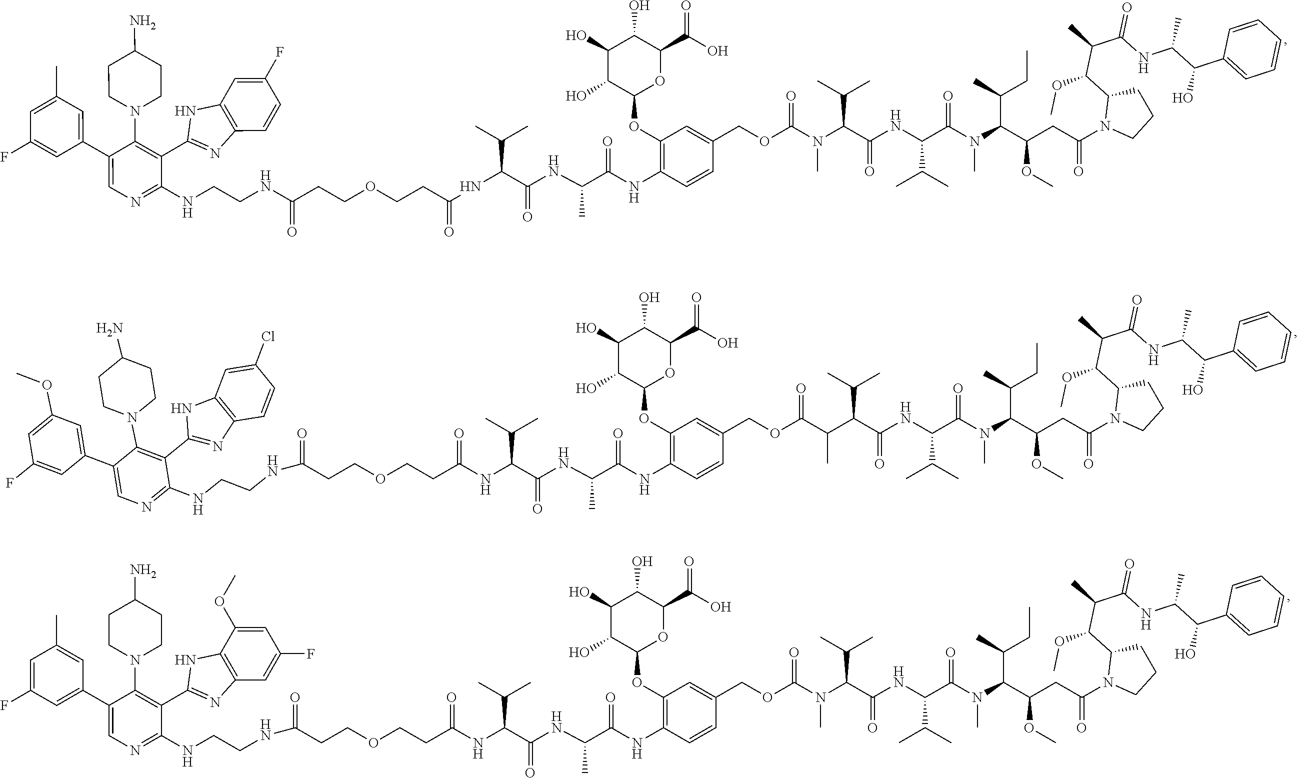

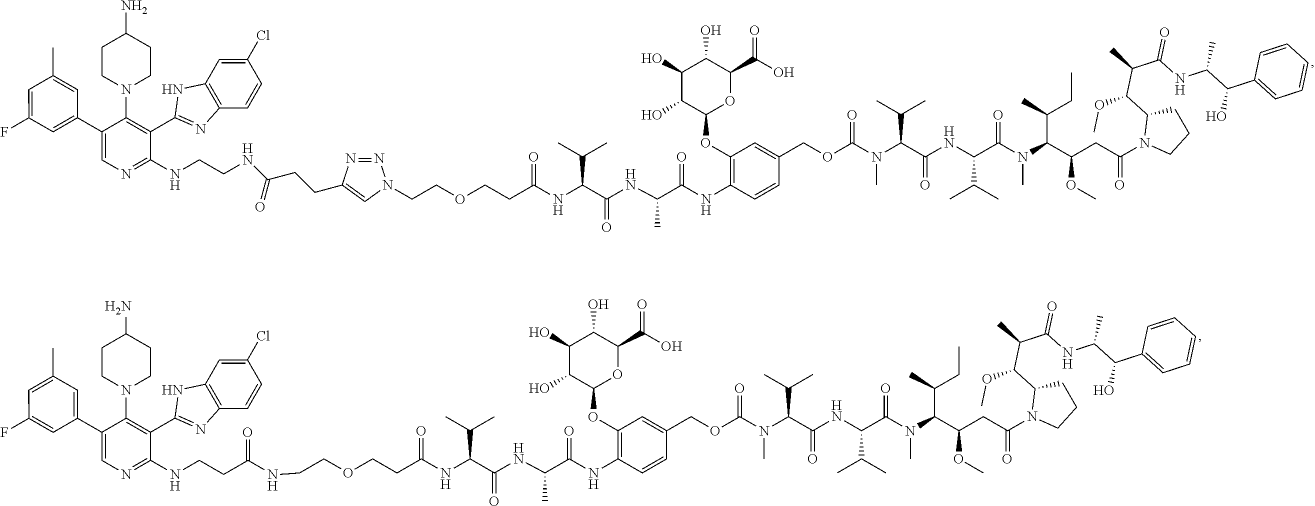

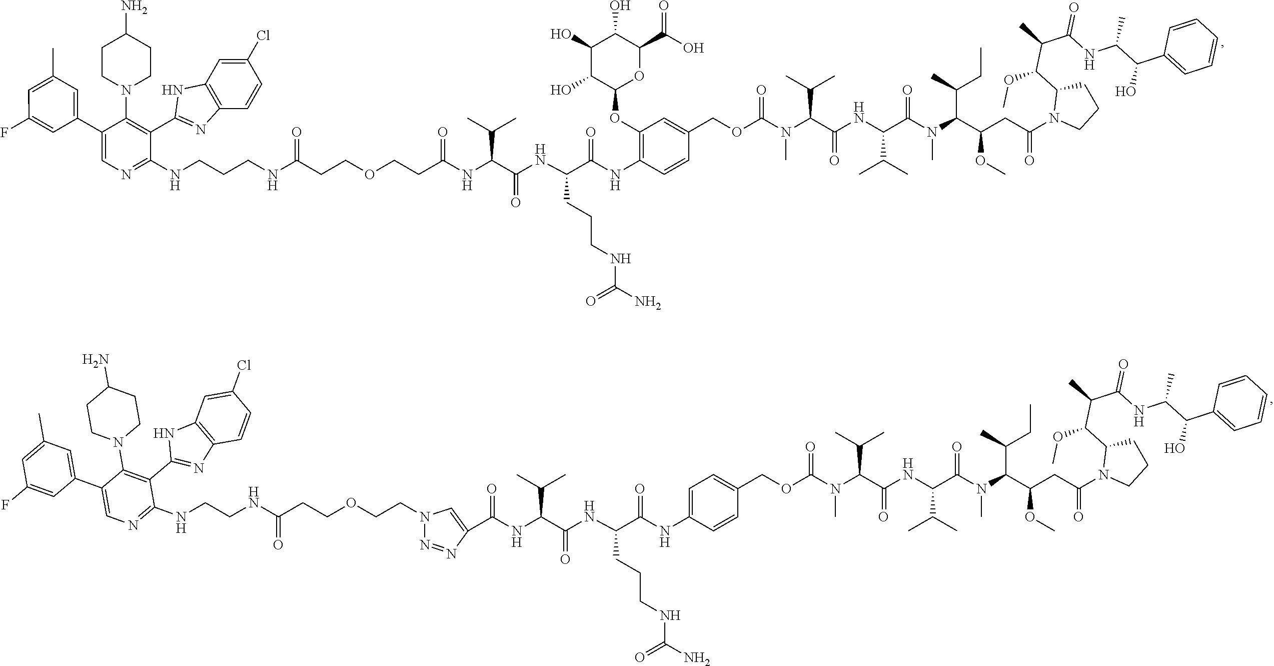

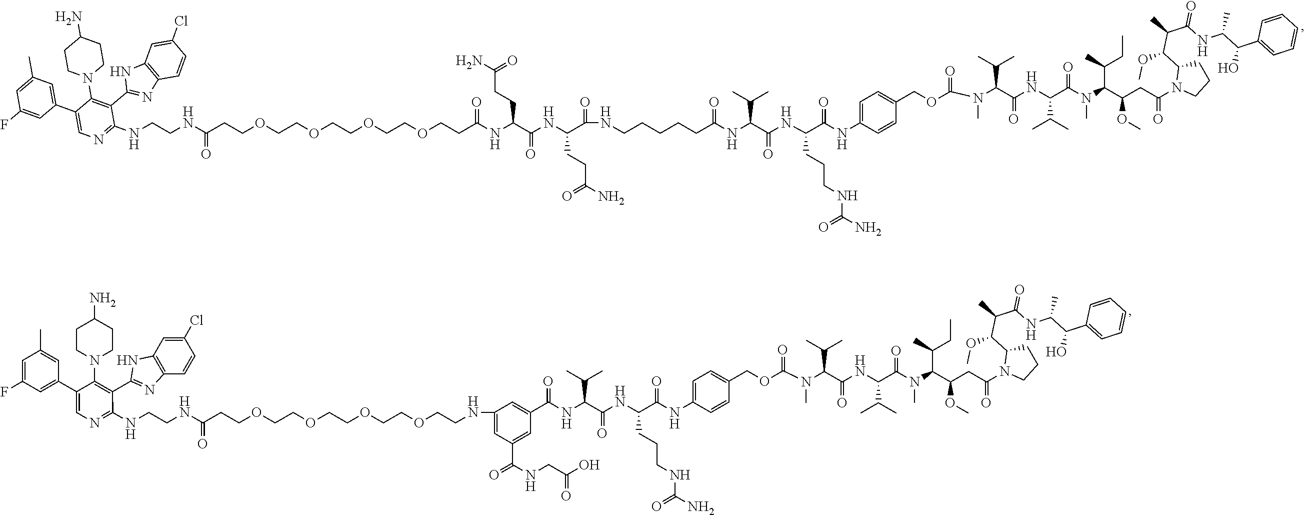

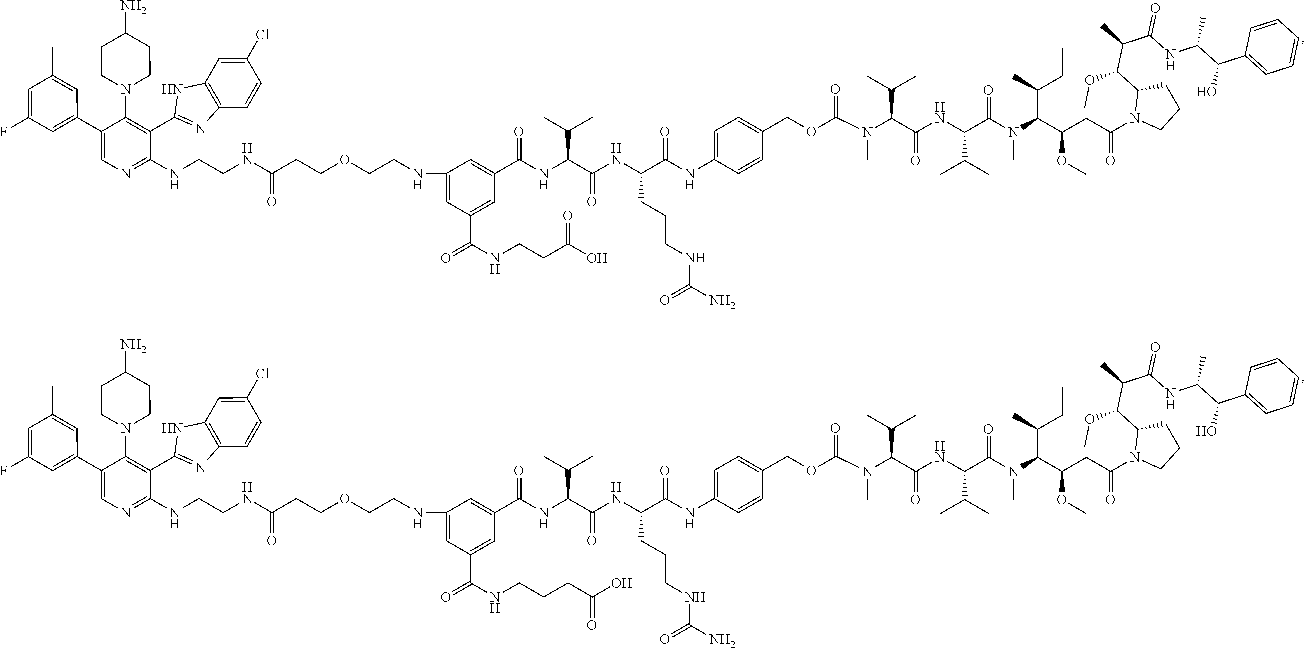

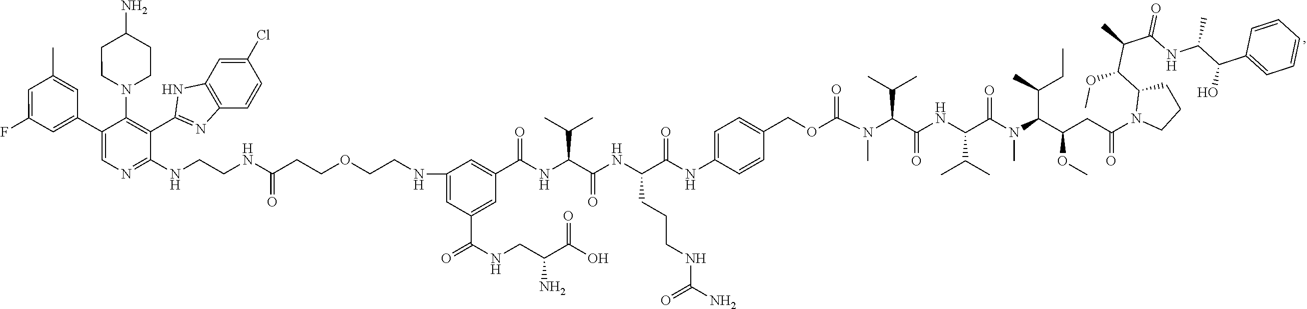 [SMILES: CC[C@H](C)[C@@H]([C@@H](CC(=O)N1CCC[C@H]1[C@H](OC)[C@@H](C)C(=O)N[C@H](C)[C@@H](O)c1ccccc1)OC)N(C)C(=O)[C@@H](NC(=O)[C@H](C(C)C)N(C)C(=O)OCc1ccc(NC(=O)[C@H](CCCNC(N)=O)NC(=O)[C@@H](NC(=O)c2cc(NCCOCCC(=O)NCCNc3ncc(-c4cc(C)cc(F)c4)c(N4CCC(N)CC4)c3-c3nc4ccc(Cl)cc4[nH]3)cc(C(=O)NC[C@@H](N)C(=O)O)c2)C(C)C)cc1)C(C)C]

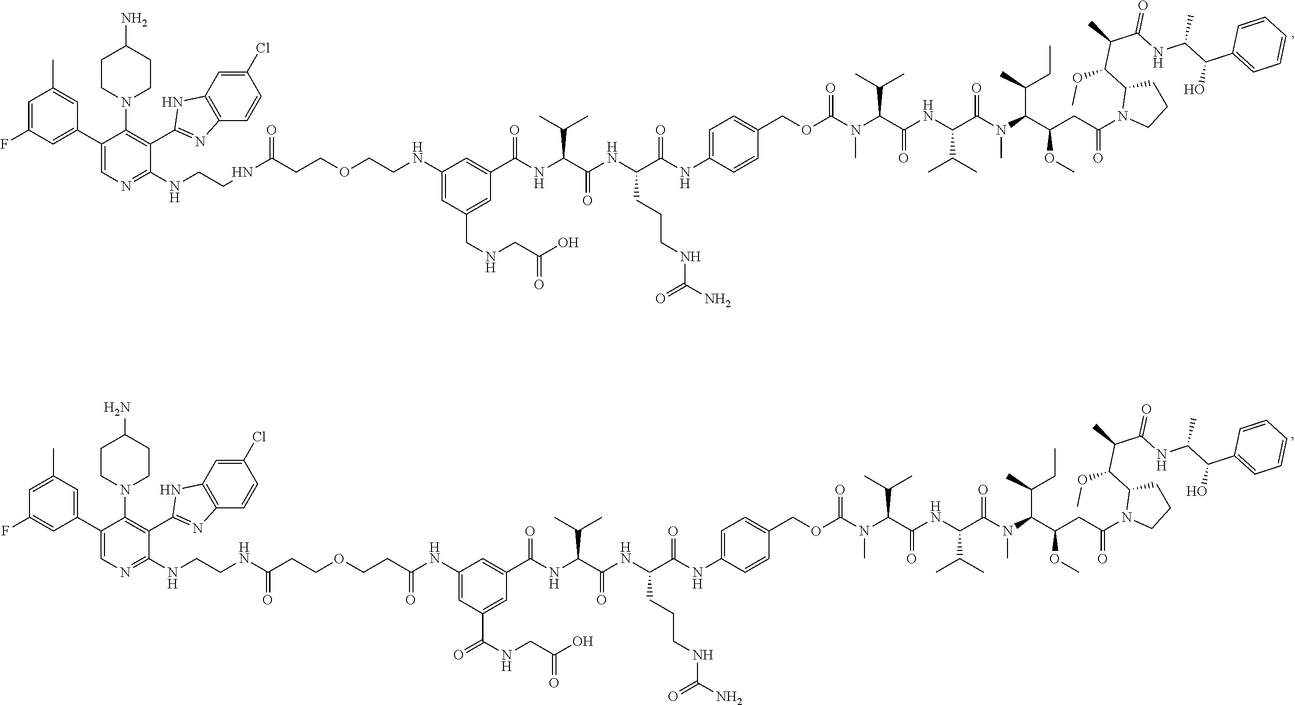

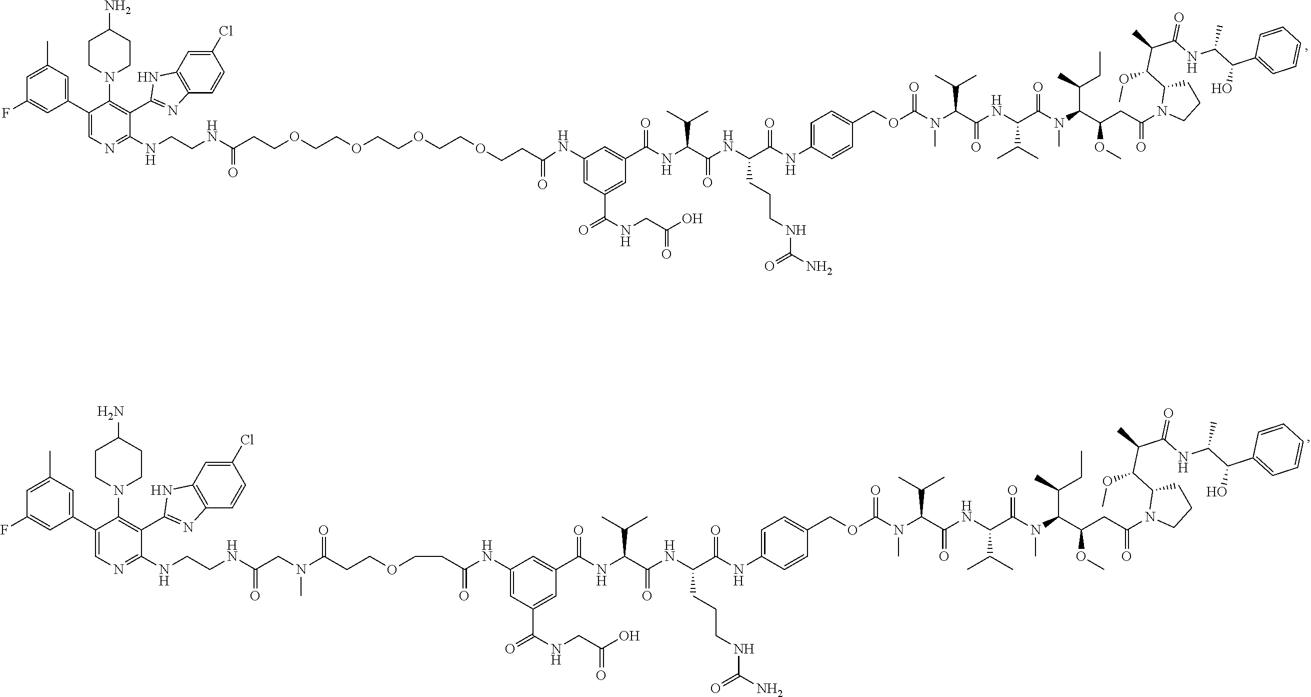

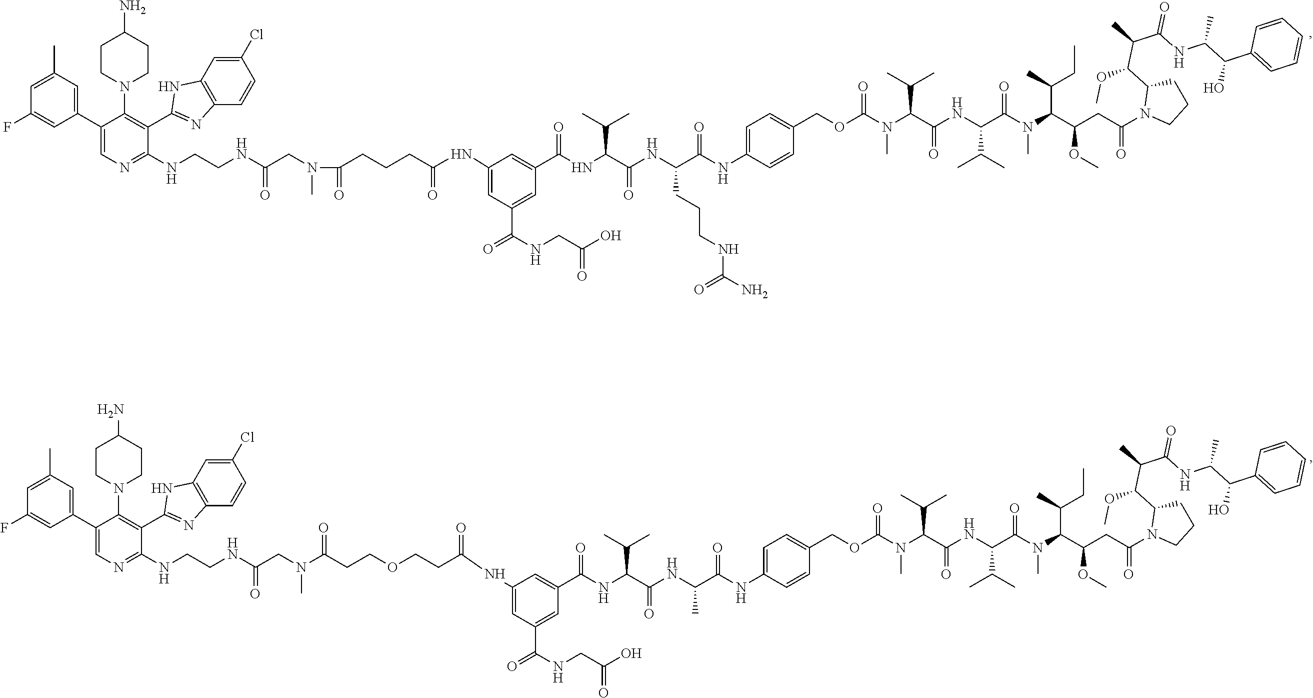

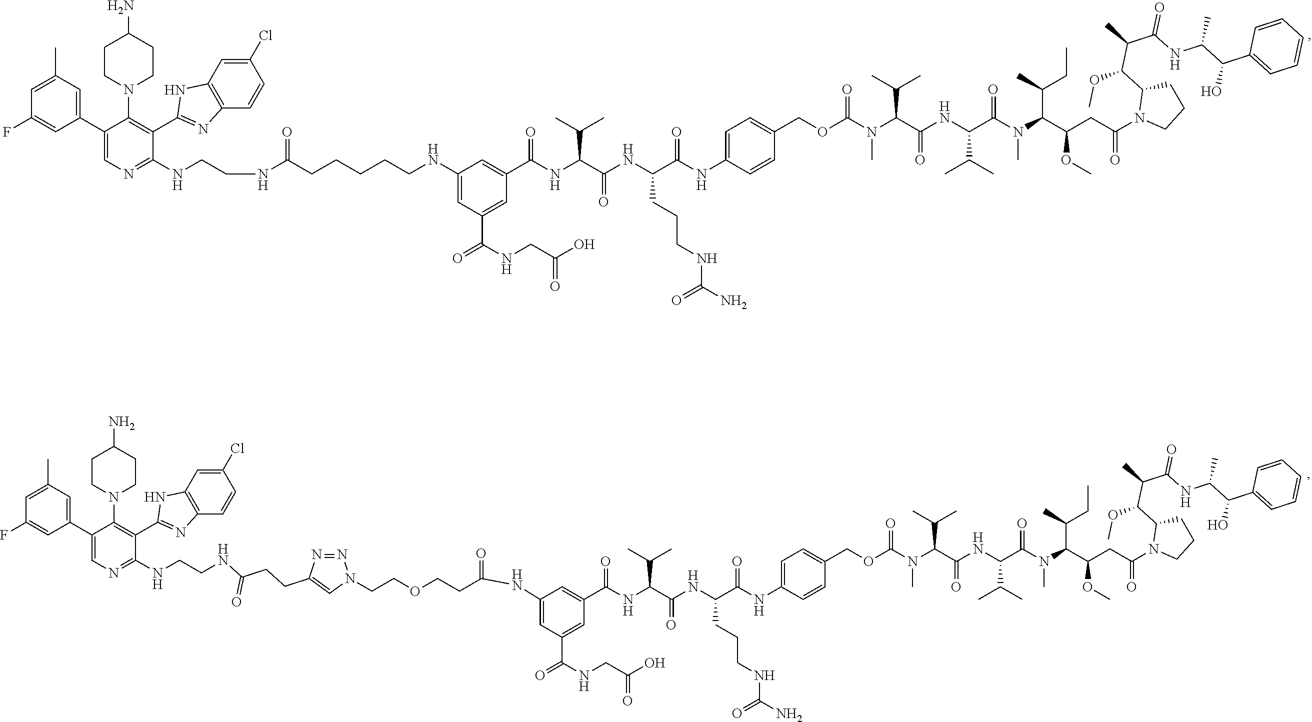

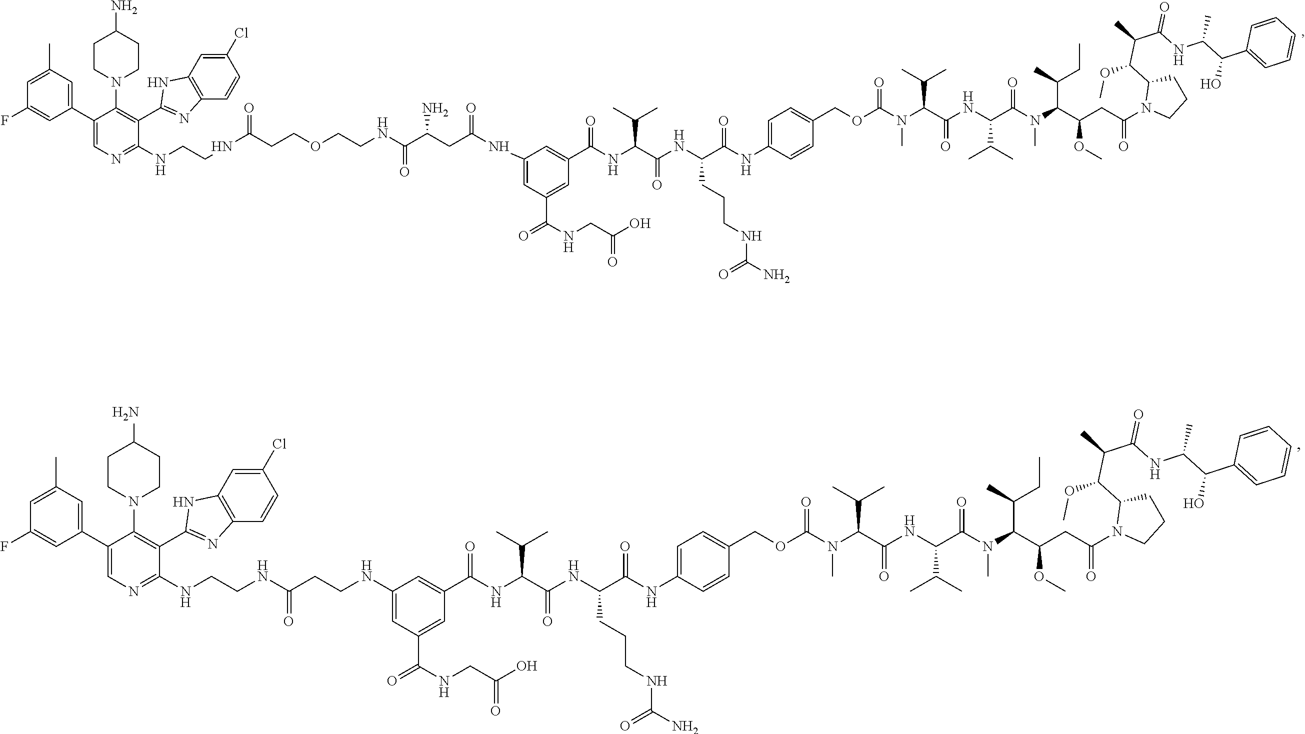

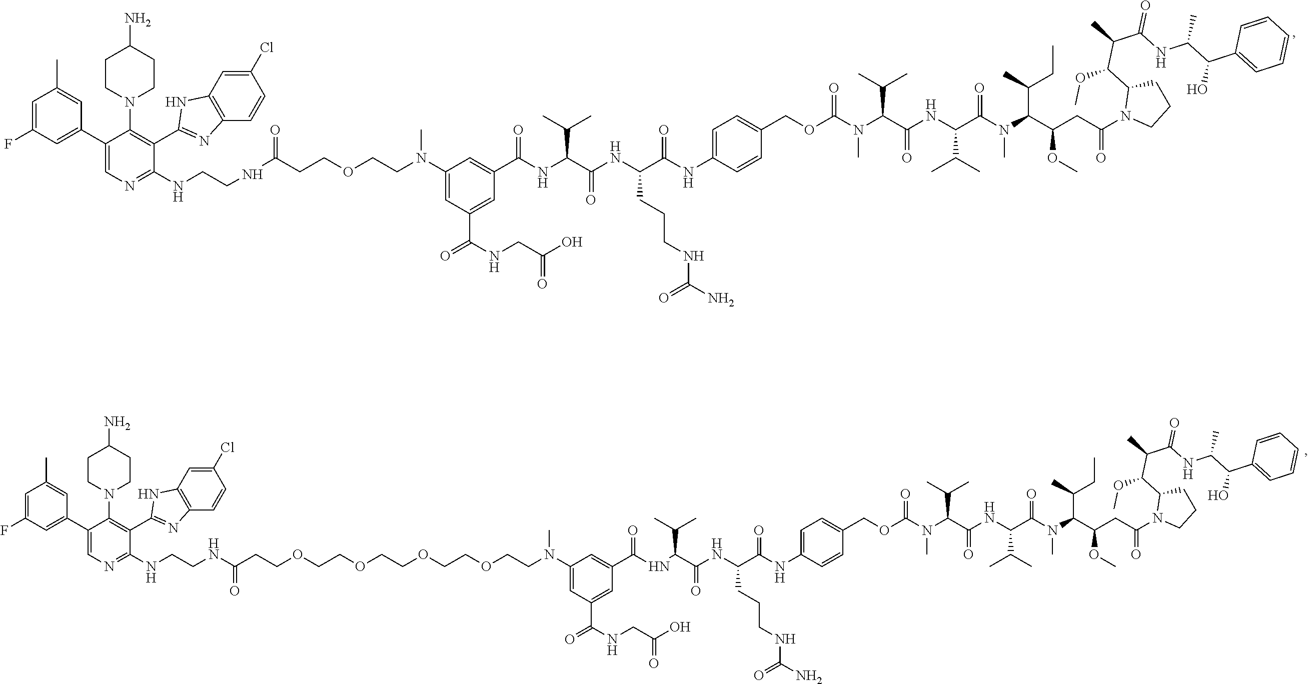

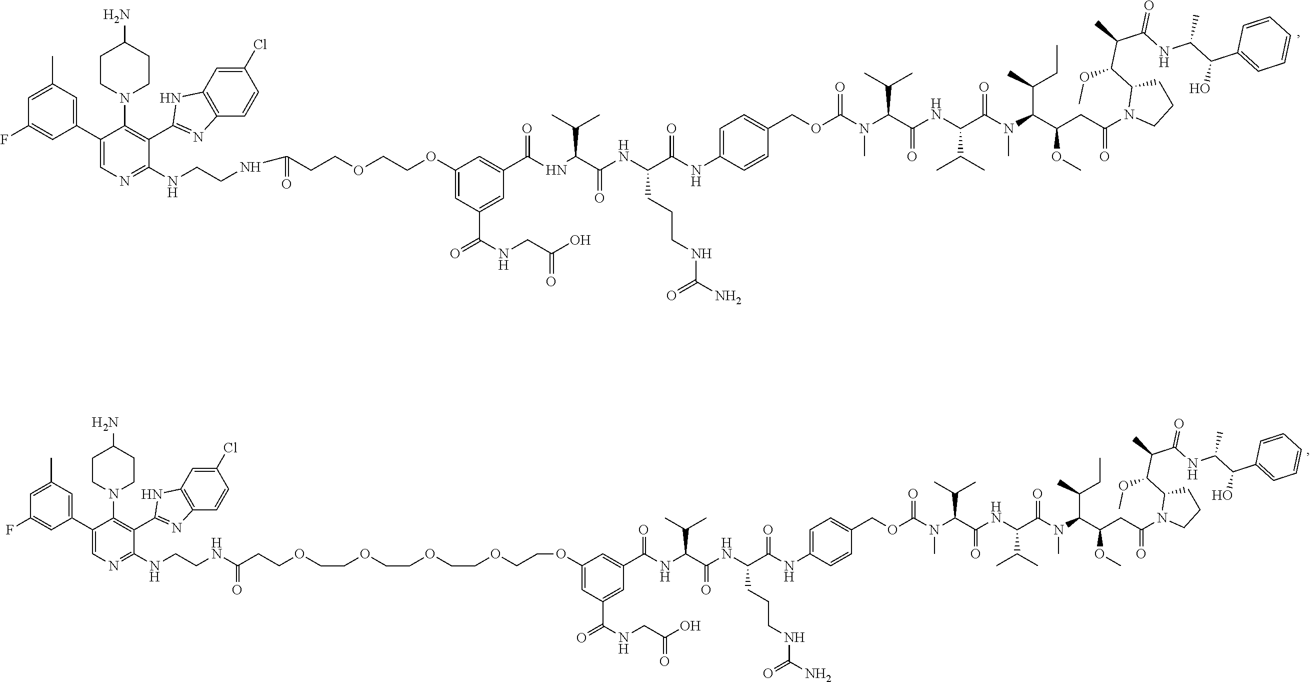

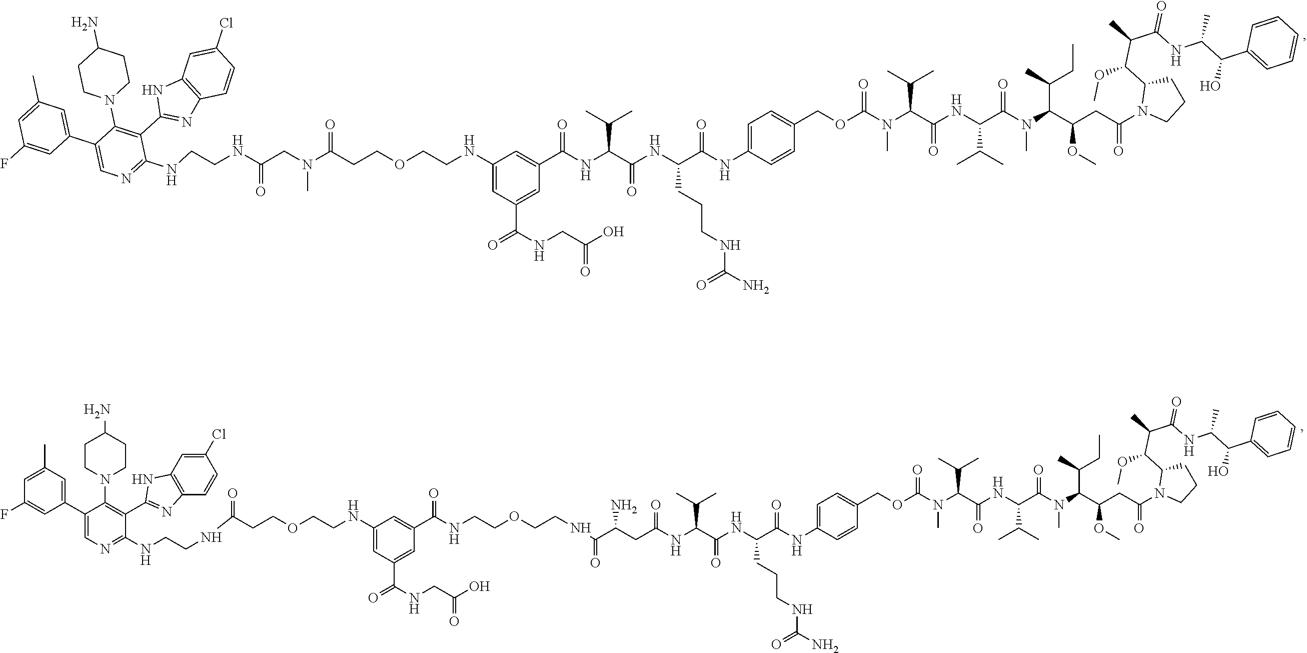

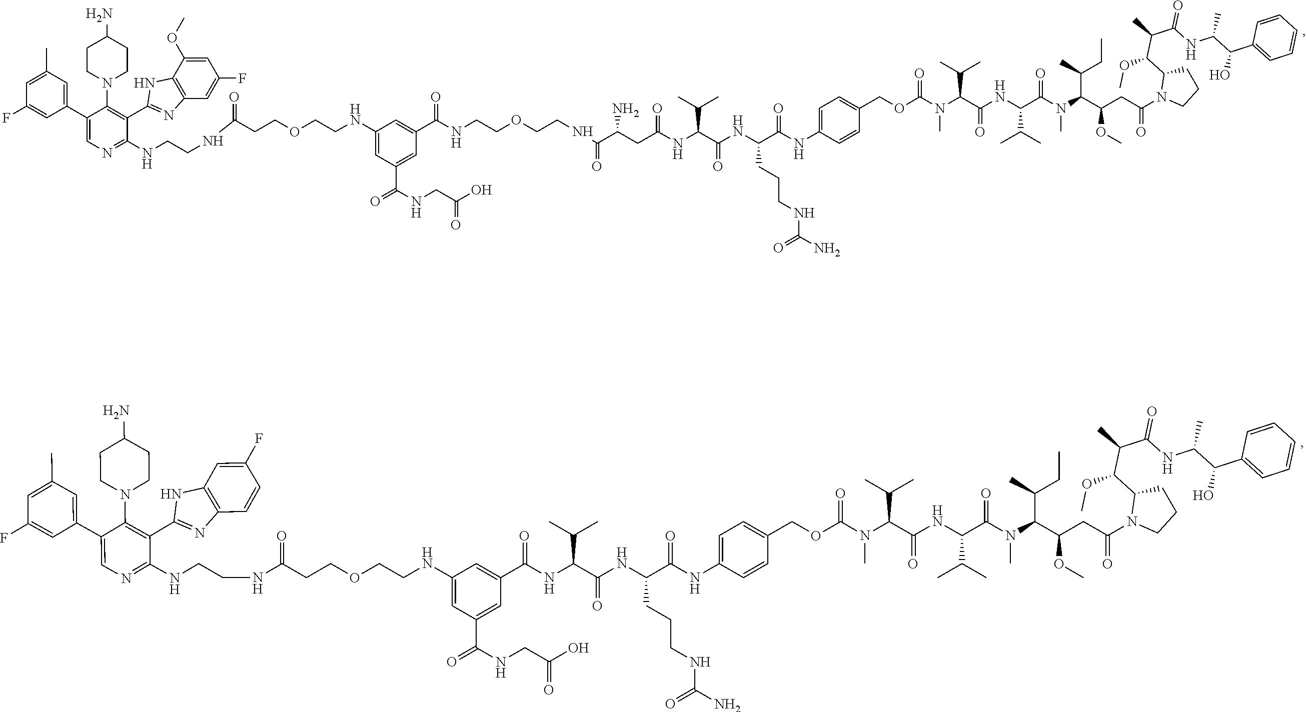

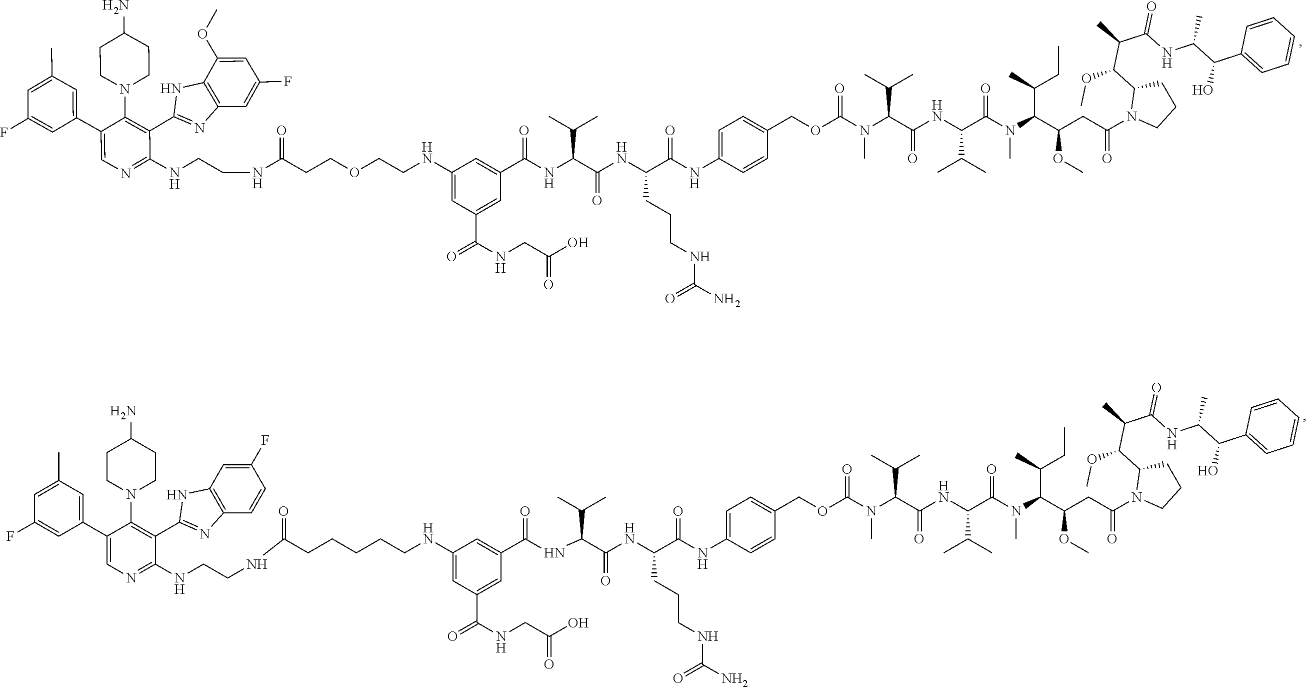

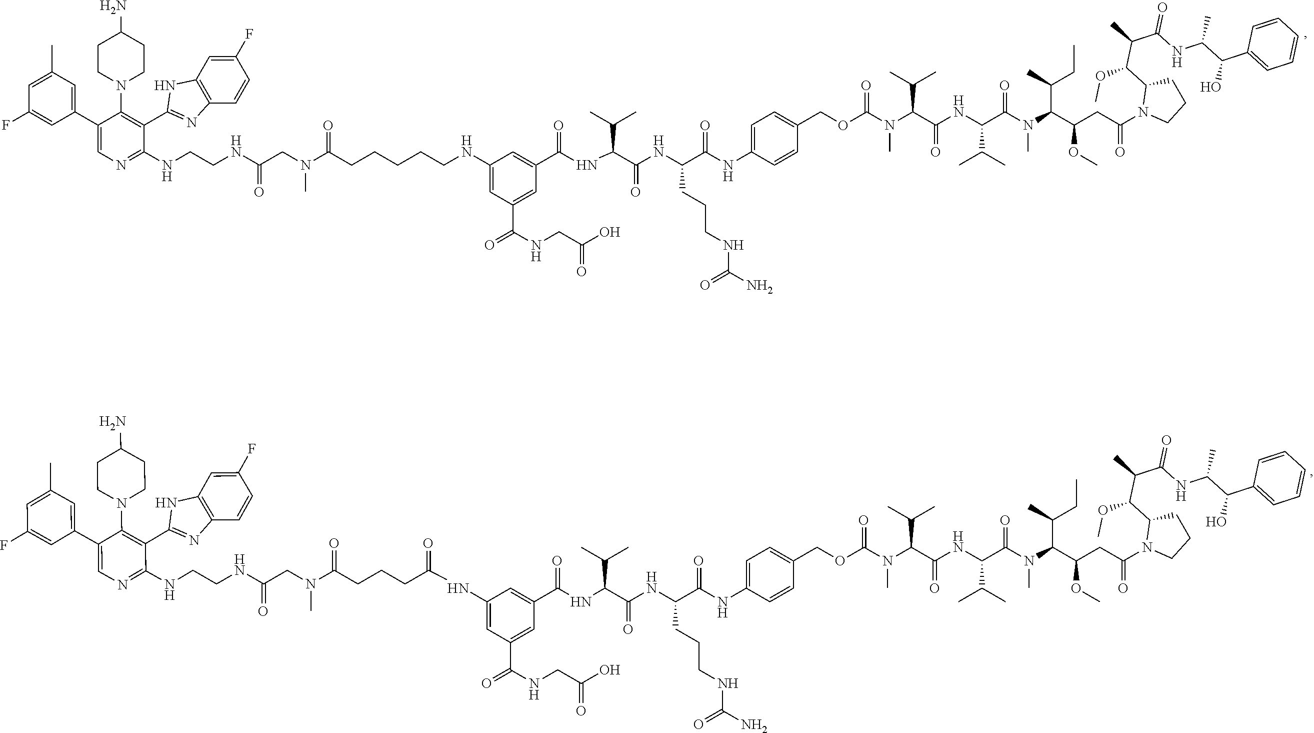

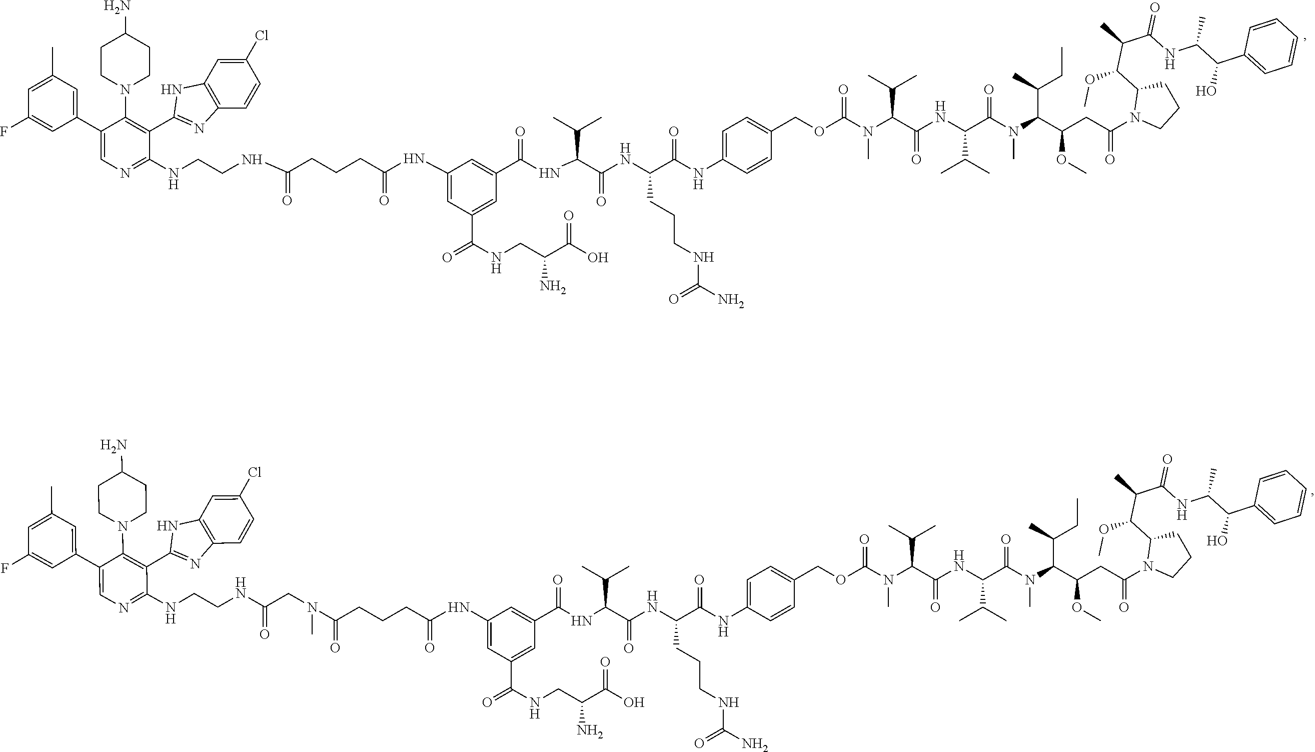

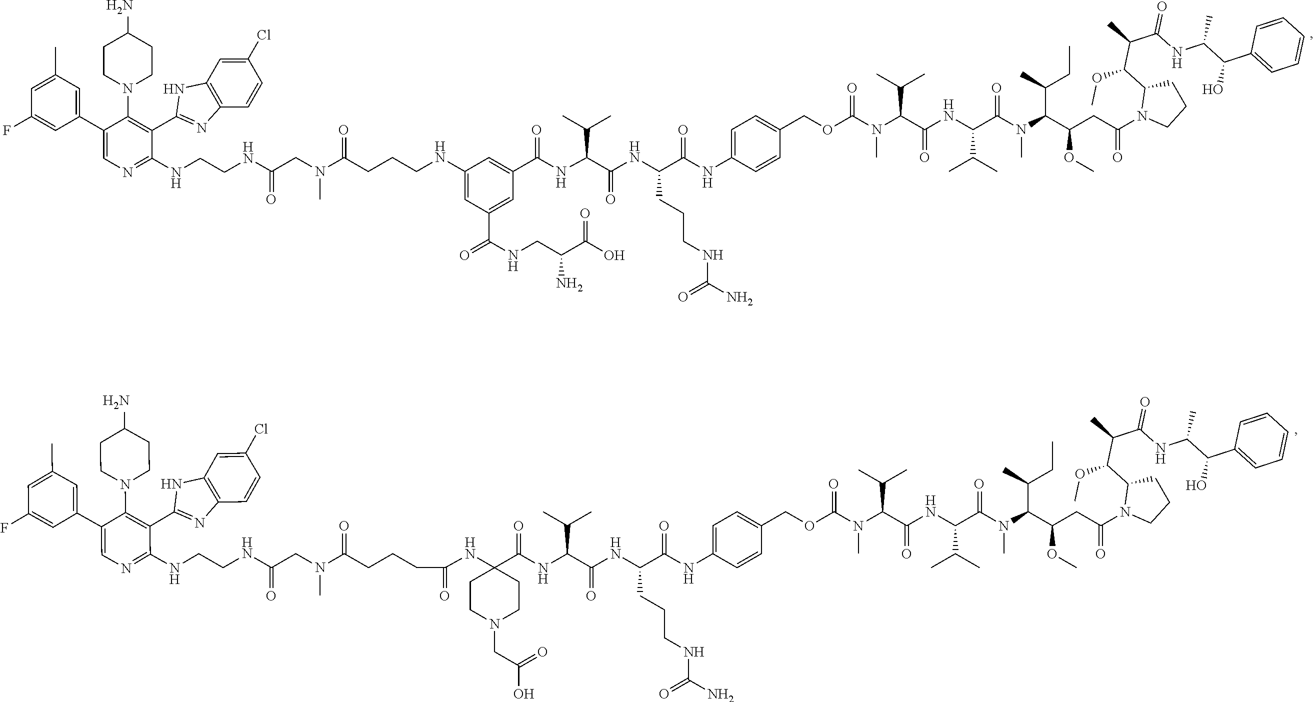

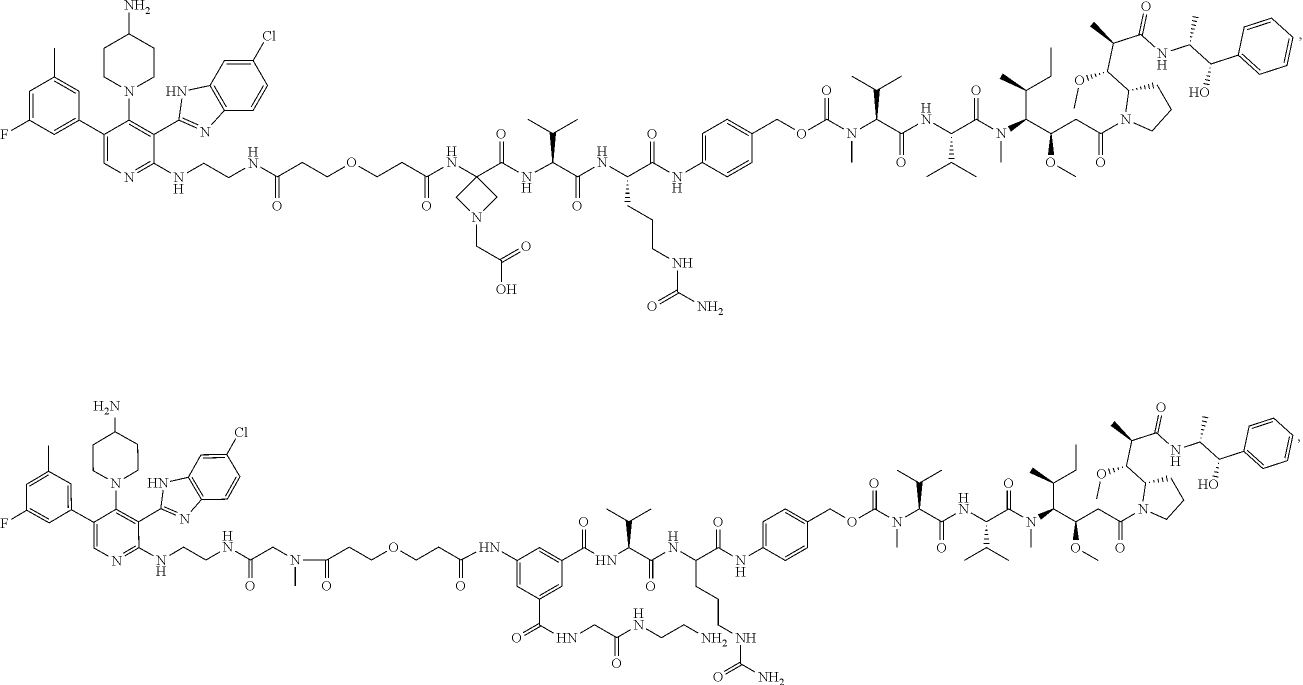

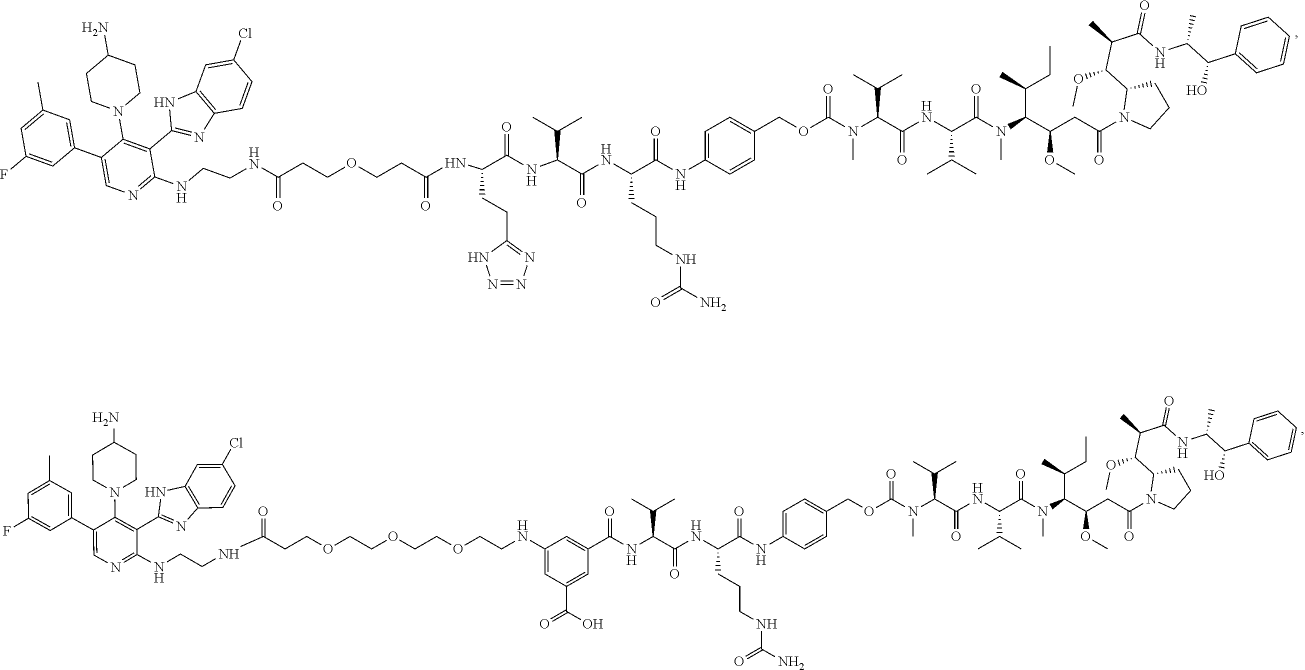

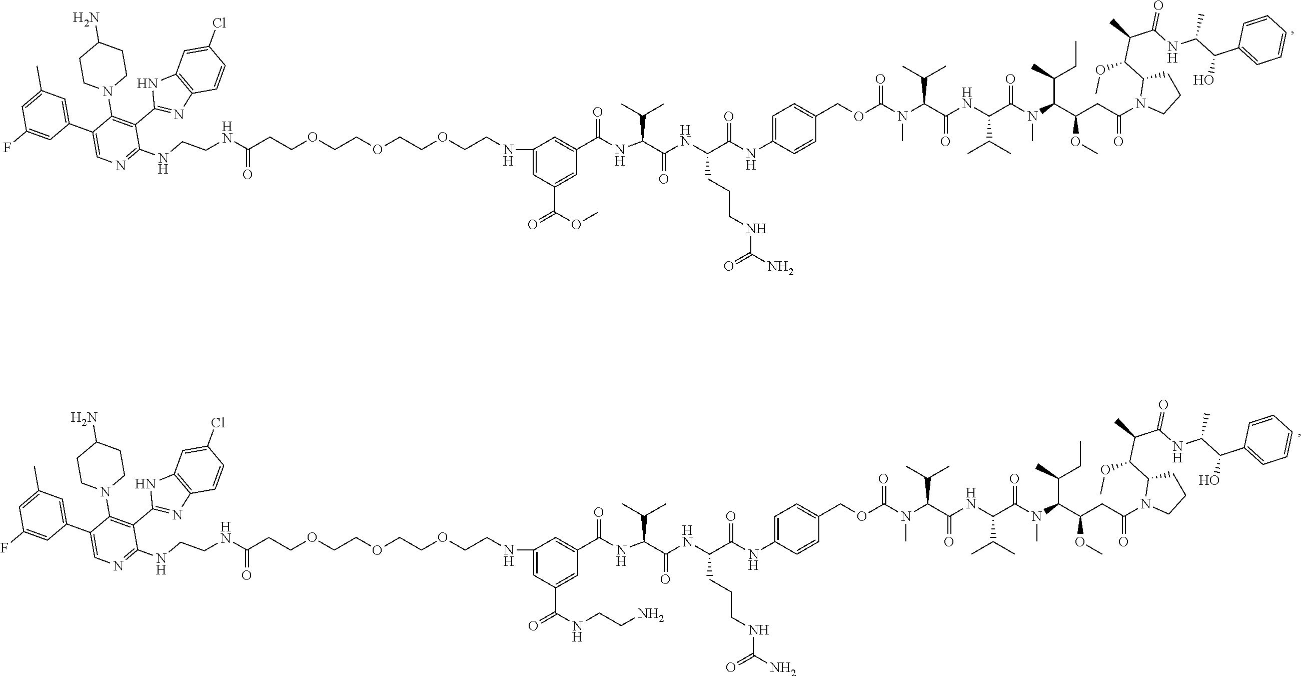

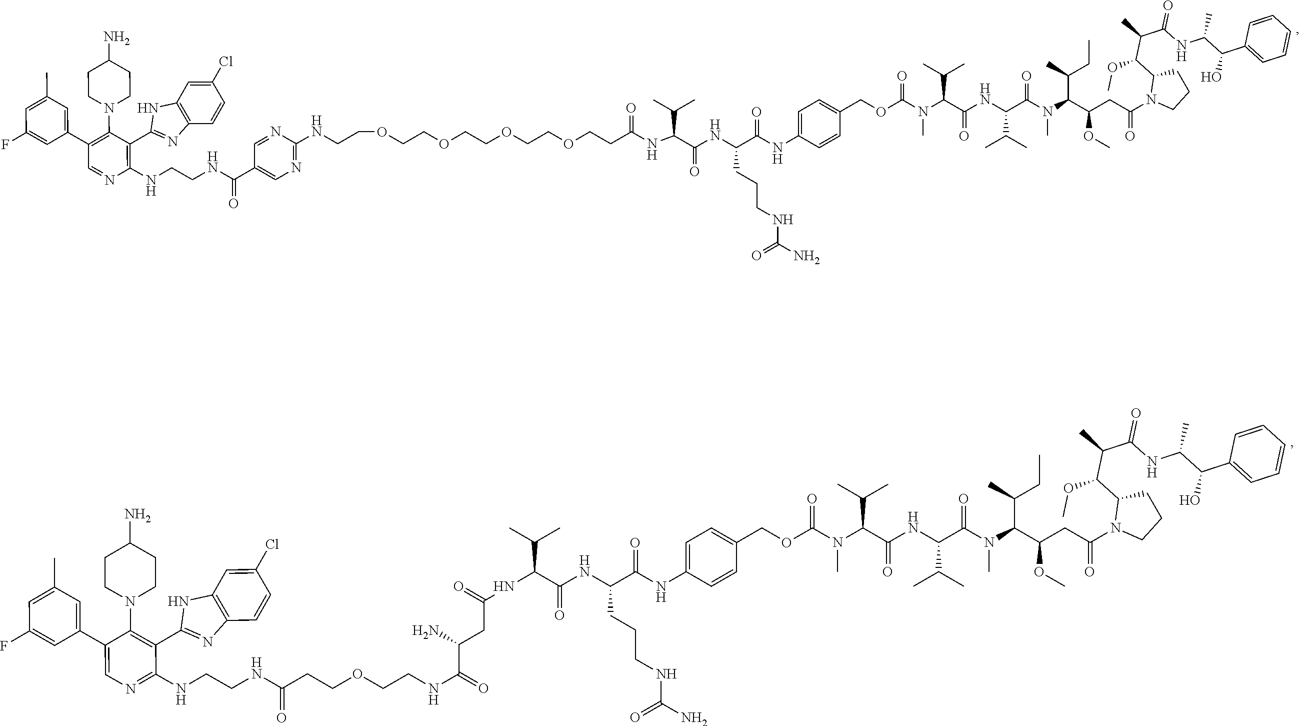

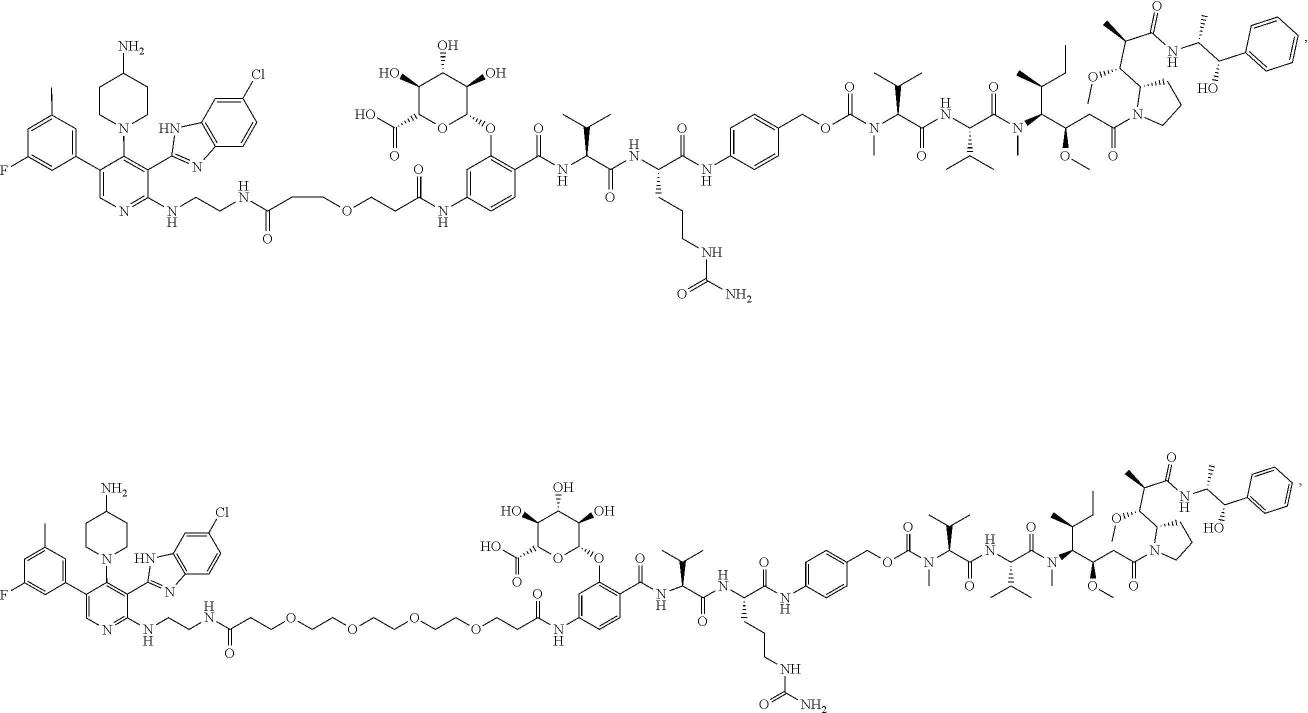

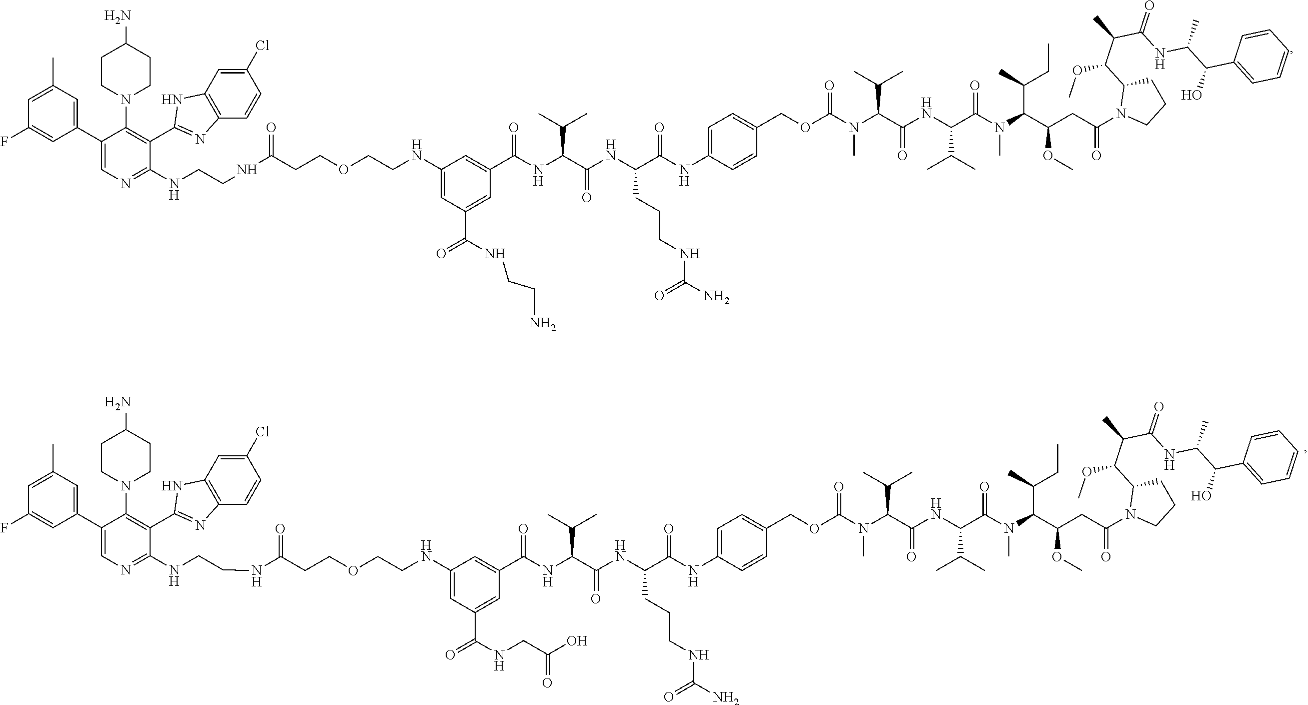

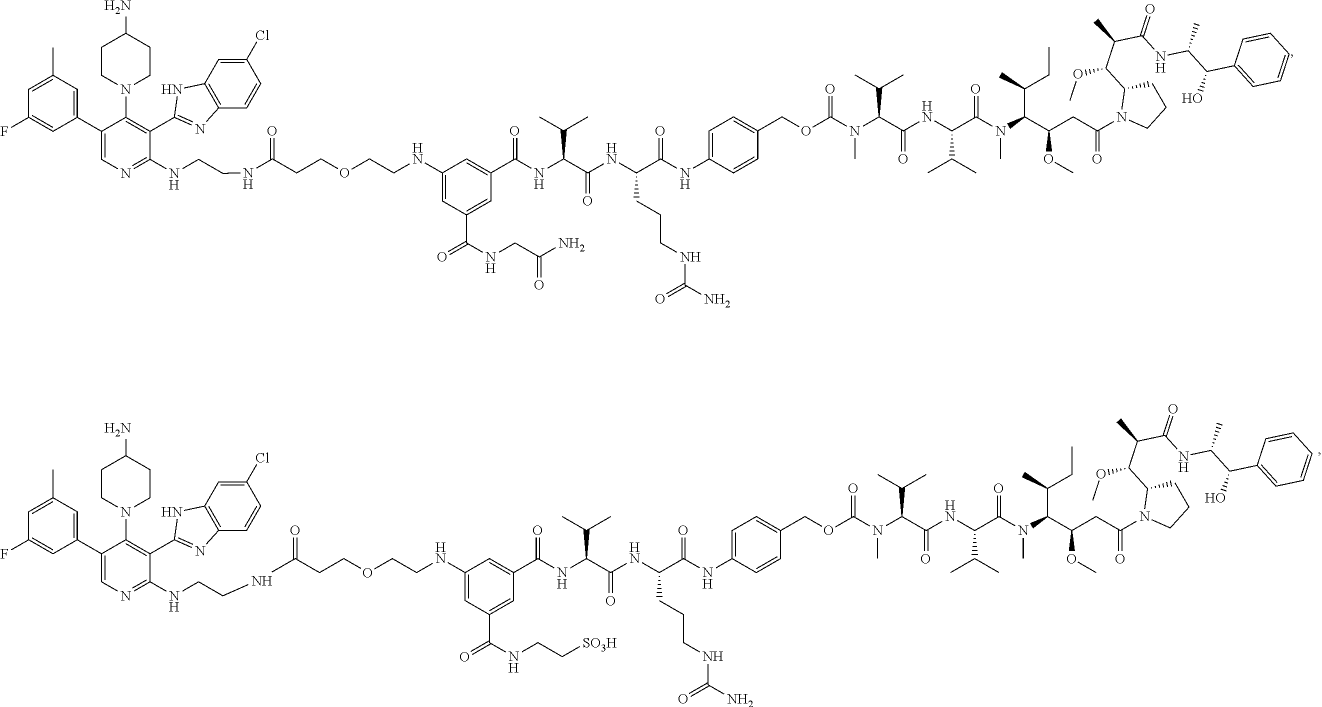

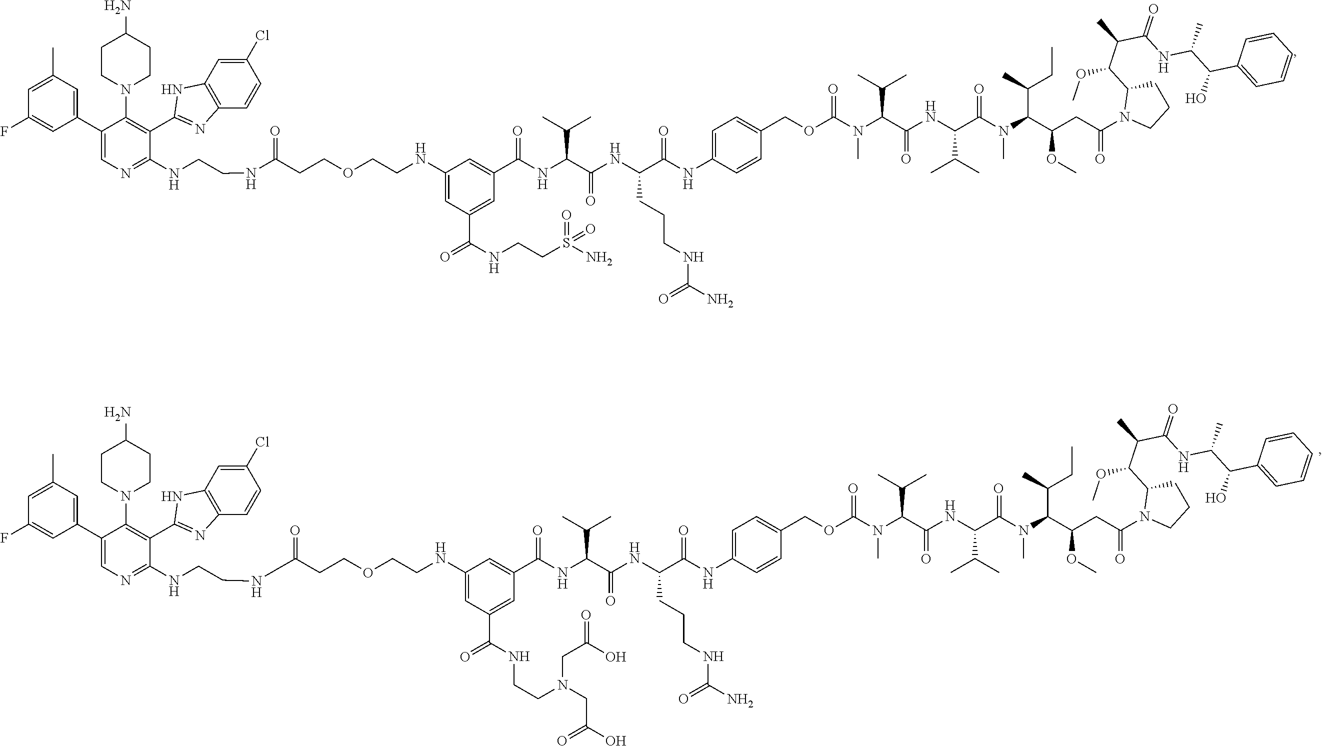

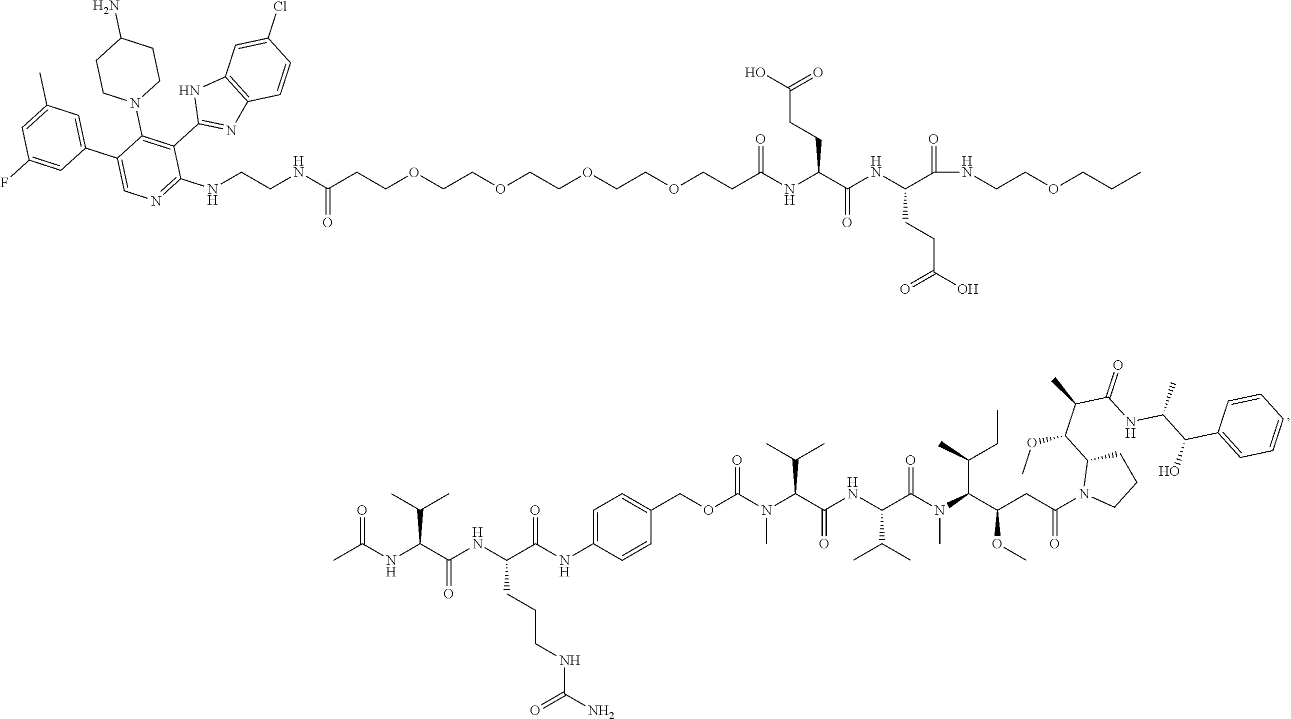

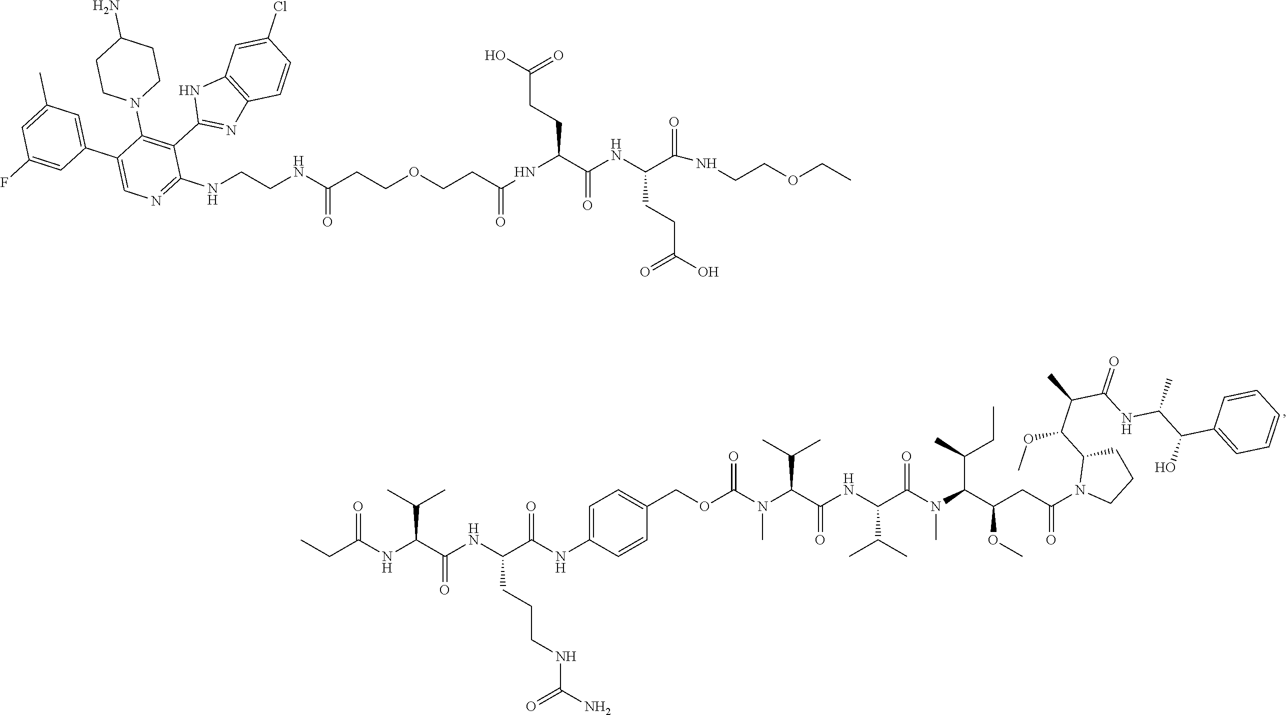

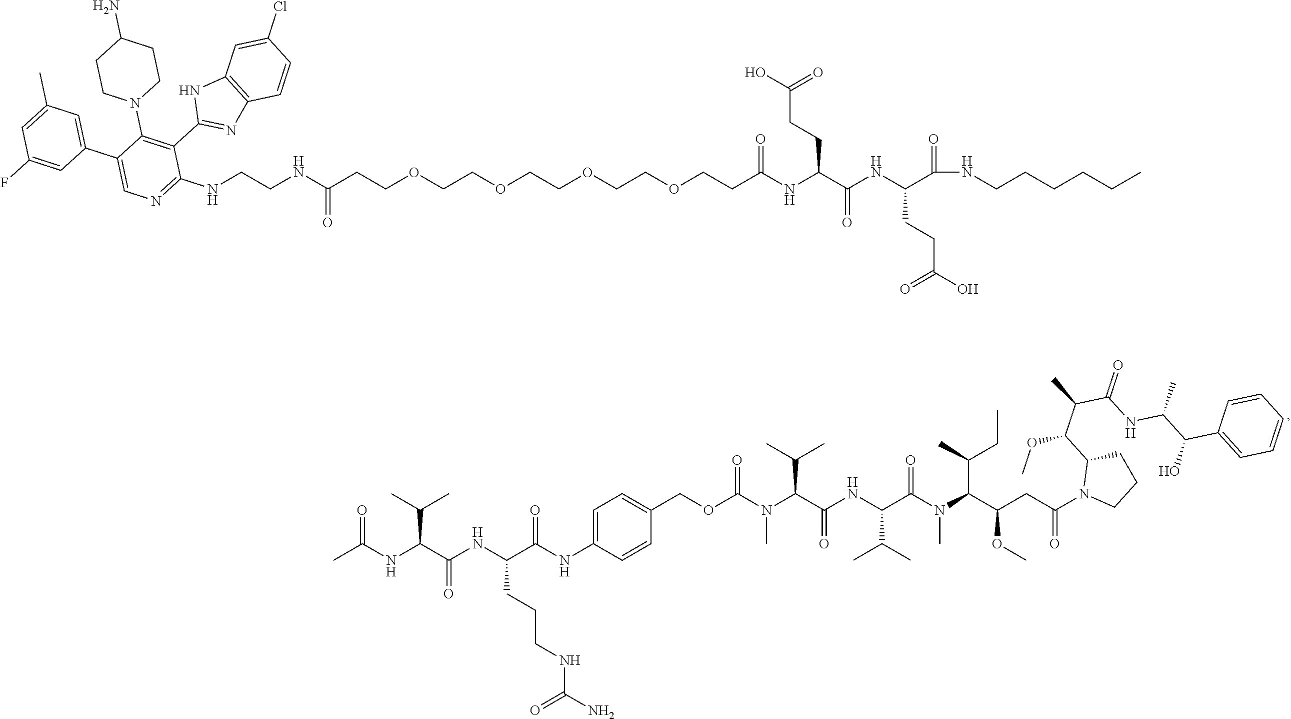

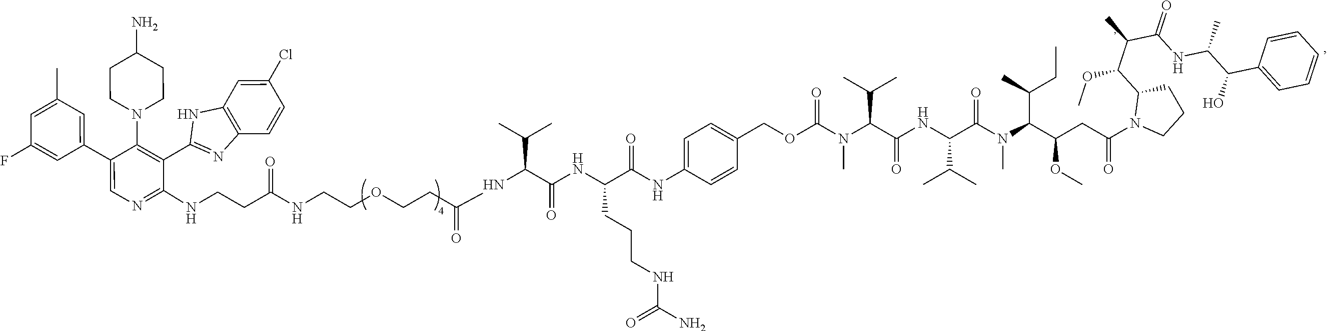

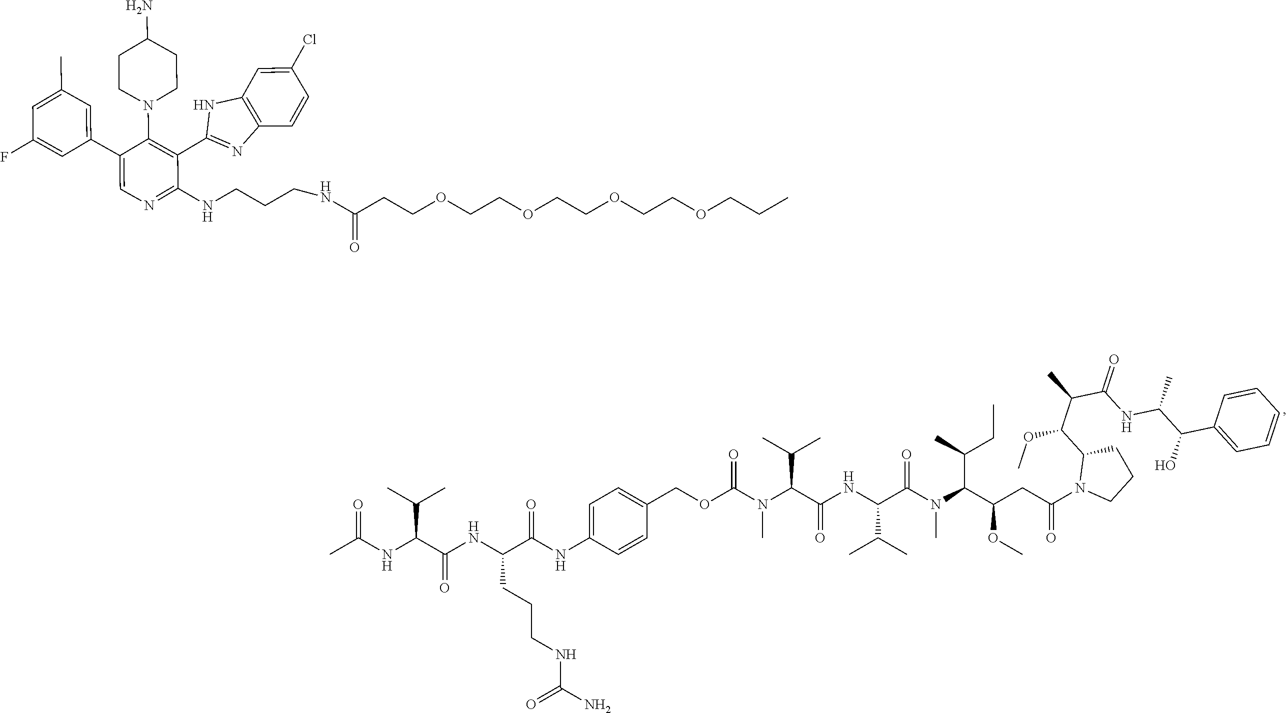

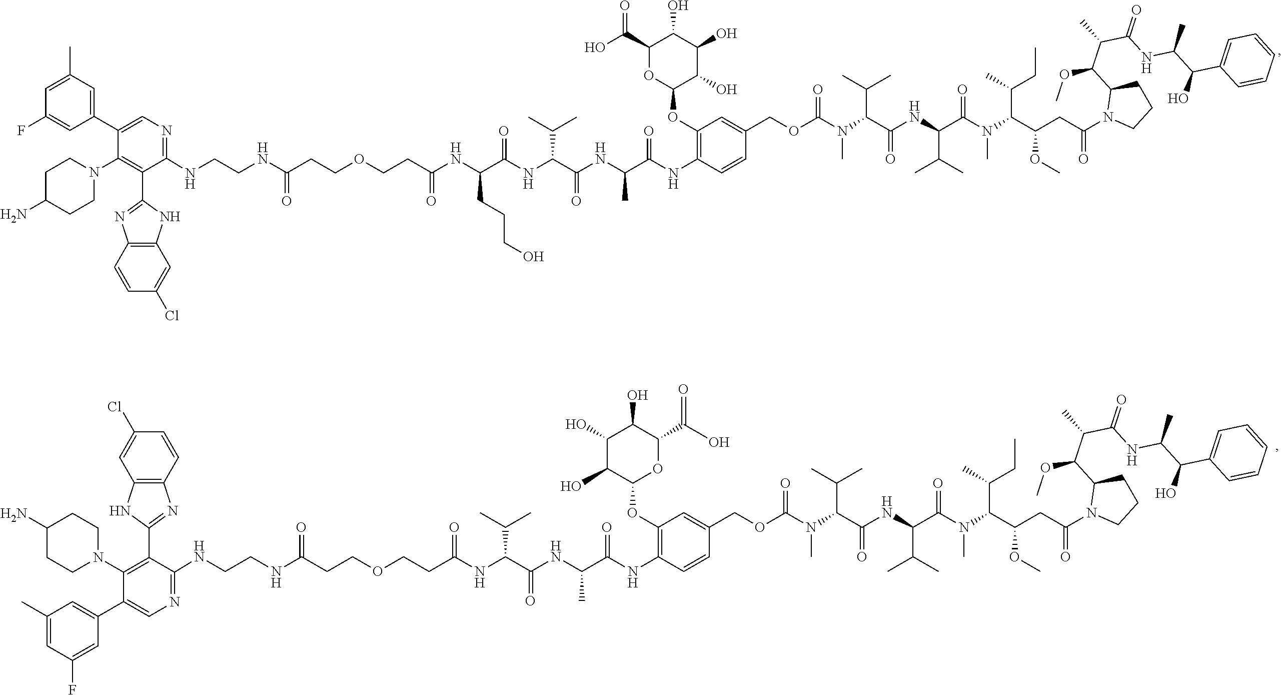

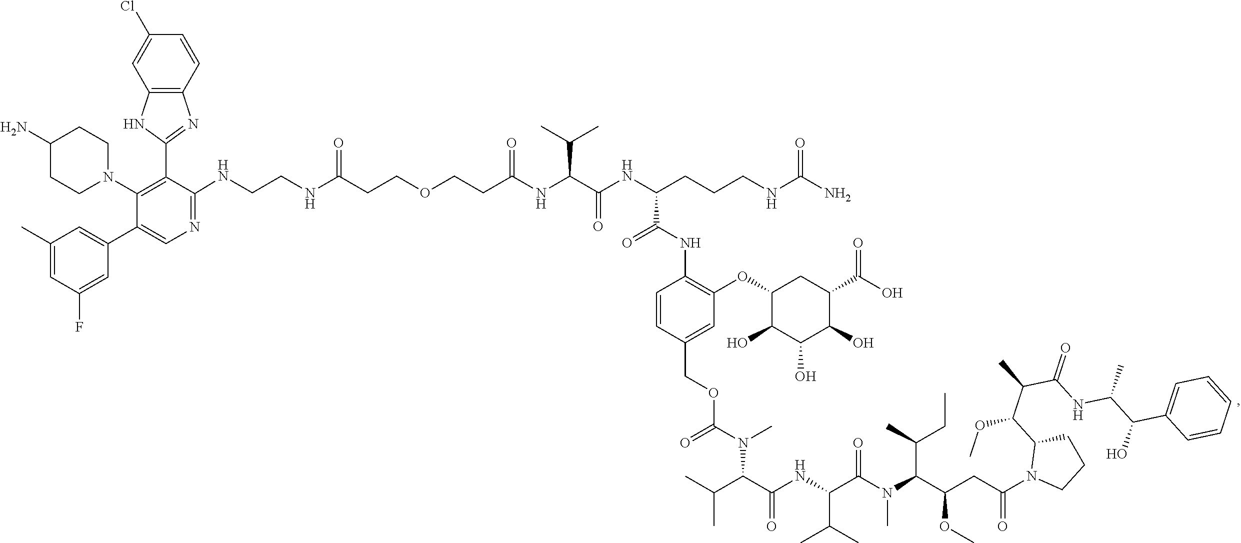 [SMILES: CC[C@H](C)[C@@H]([C@@H](CC(=O)N1CCC[C@H]1[C@H](OC)[C@@H](C)C(=O)N[C@H](C)[C@@H](O)c1ccccc1)OC)N(C)C(=O)[C@@H](NC(=O)[C@H](C(C)C)N(C)C(=O)OCc1ccc(NC(=O)[C@@H](CCCNC(N)=O)NC(=O)[C@@H](NC(=O)CCOCCC(=O)NCCNc2ncc(-c3cc(C)cc(F)c3)c(N3CCC(N)CC3)c2-c2nc3ccc(Cl)cc3[nH]2)C(C)C)c(O[C@@H]2C[C@H](C(=O)O)[C@@H](O)[C@H](O)[C@H]2O)c1)C(C)C]

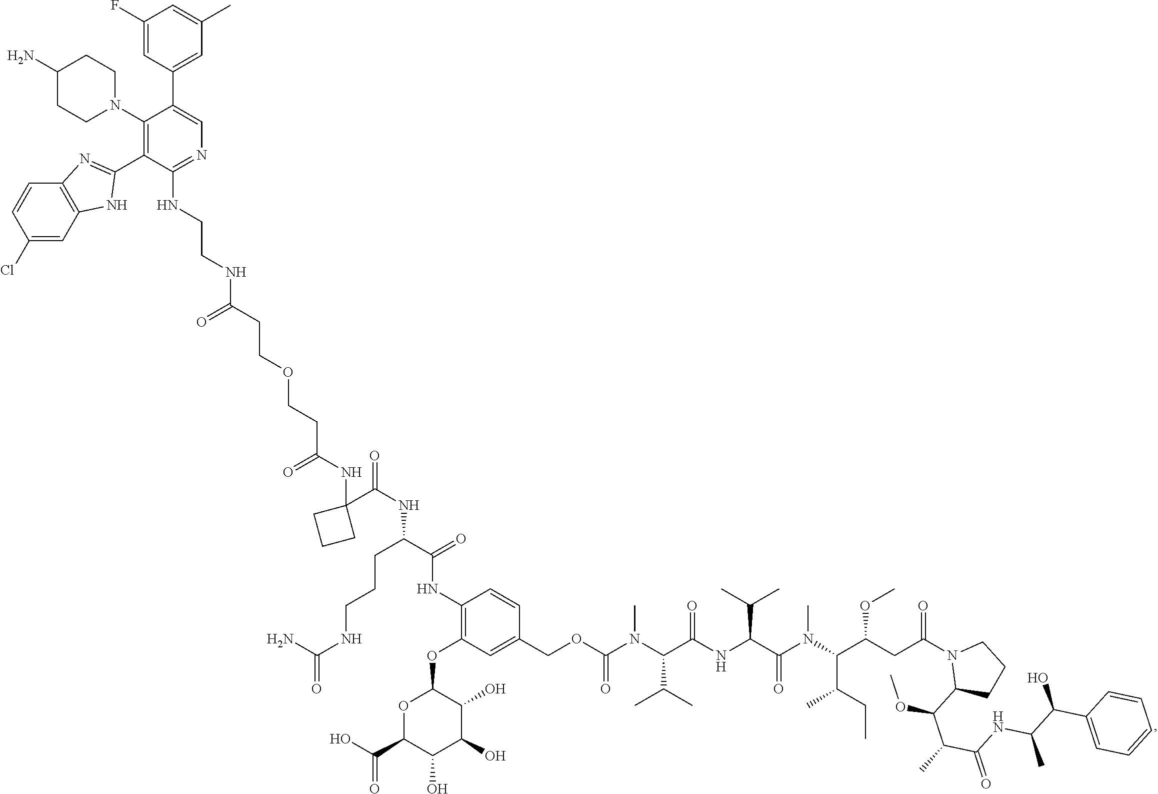 [SMILES: CC[C@H](C)[C@@H]([C@@H](CC(=O)N1CCC[C@H]1[C@H](OC)[C@@H](C)C(=O)N[C@H](C)[C@@H](O)c1ccccc1)OC)N(C)C(=O)[C@@H](NC(=O)[C@H](C(C)C)N(C)C(=O)OCc1ccc(NC(=O)[C@H](CCCNC(N)=O)NC(=O)C2(NC(=O)CCOCCC(=O)NCCNc3ncc(-c4cc(C)cc(F)c4)c(N4CCC(N)CC4)c3-c3nc4ccc(Cl)cc4[nH]3)CCC2)c(O[C@@H]2O[C@H](C(=O)O)[C@@H](O)[C@H](O)[C@H]2O)c1)C(C)C]

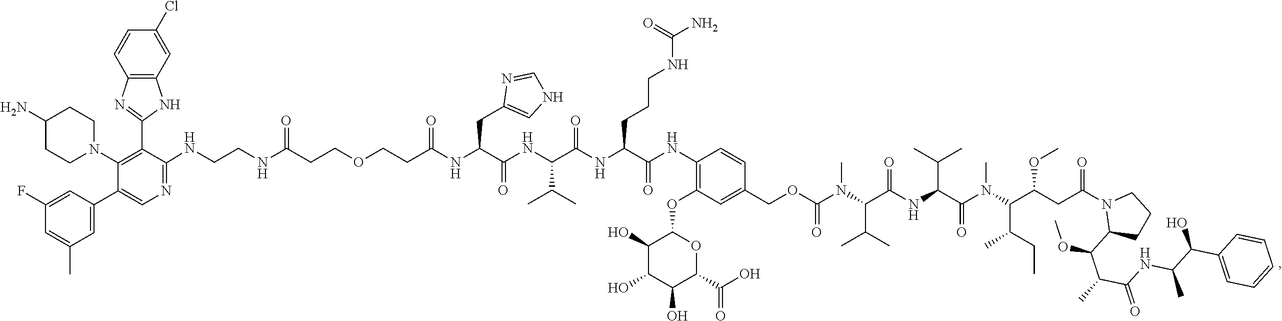

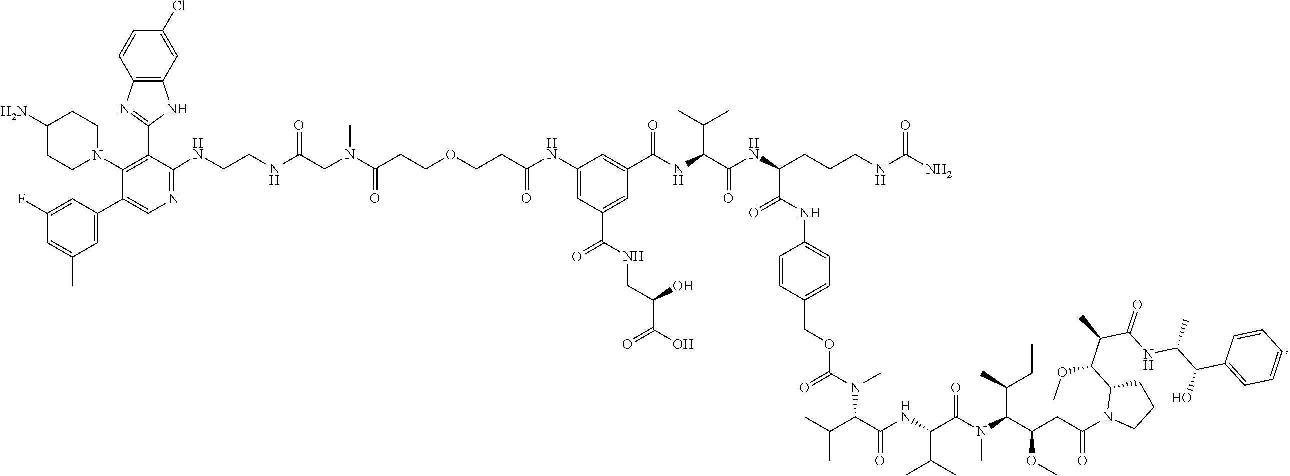 [SMILES: CC[C@H](C)[C@@H]([C@@H](CC(=O)N1CCC[C@H]1[C@H](OC)[C@@H](C)C(=O)N[C@H](C)[C@@H](O)c1ccccc1)OC)N(C)C(=O)[C@@H](NC(=O)[C@H](C(C)C)N(C)C(=O)OCc1ccc(NC(=O)[C@H](CCCNC(N)=O)NC(=O)[C@@H](NC(=O)c2cc(NC(=O)CCOCCC(=O)N(C)CC(=O)NCCNc3ncc(-c4cc(C)cc(F)c4)c(N4CCC(N)CC4)c3-c3nc4ccc(Cl)cc4[nH]3)cc(C(=O)NC[C@@H](O)C(=O)O)c2)C(C)C)cc1)C(C)C]

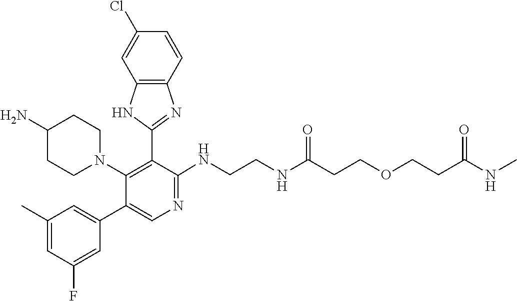

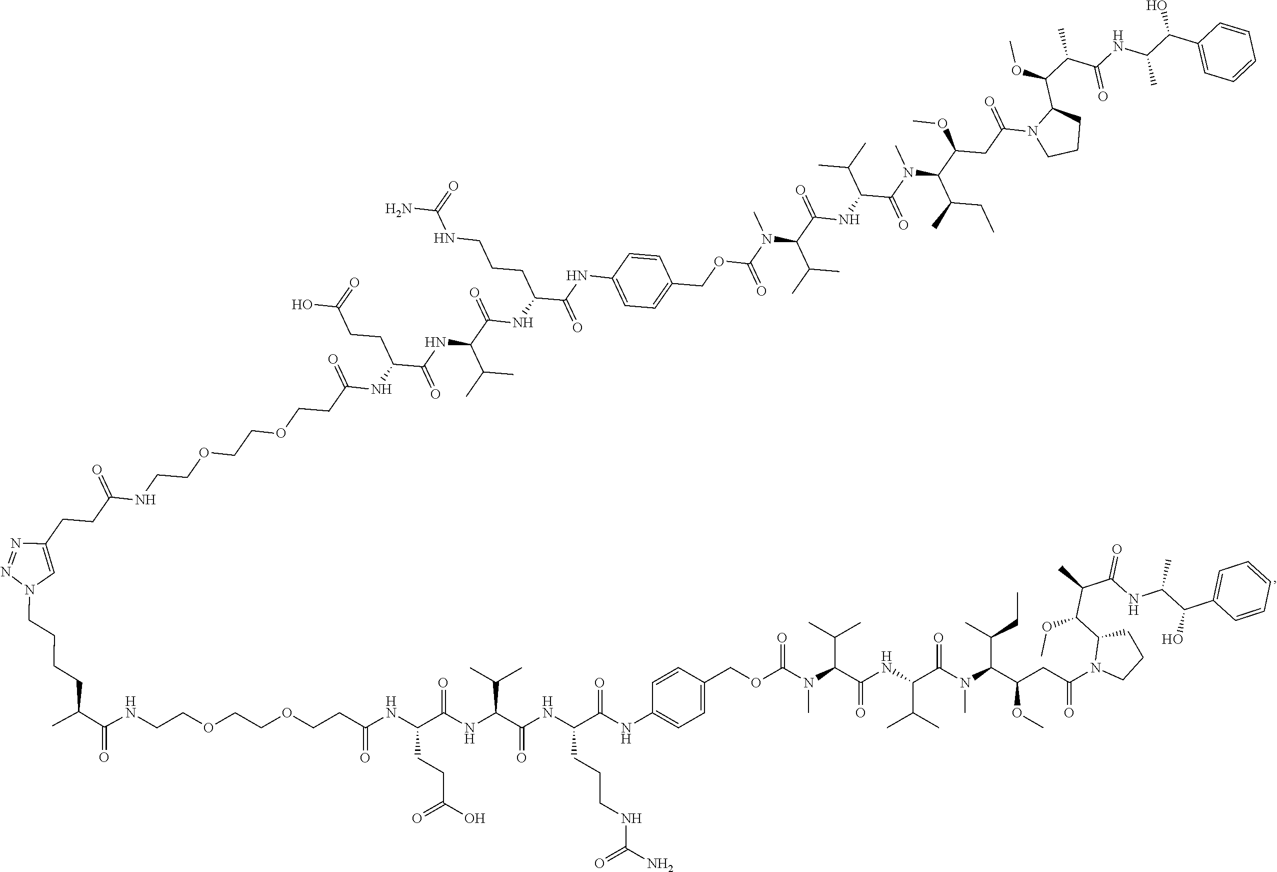

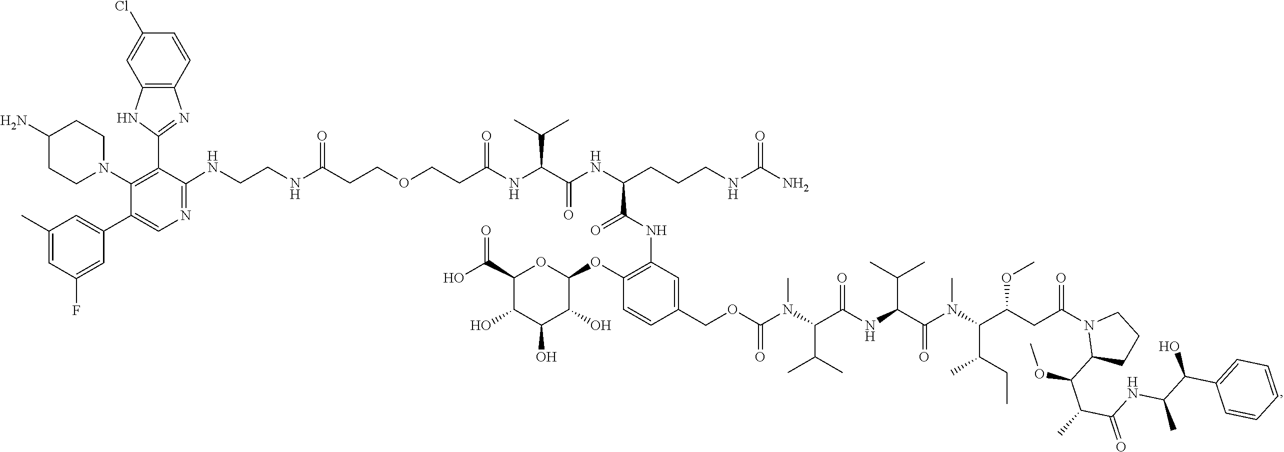 [SMILES: CC[C@H](C)[C@@H]([C@@H](CC(=O)N1CCC[C@H]1[C@H](OC)[C@@H](C)C(=O)N[C@H](C)[C@@H](O)c1ccccc1)OC)N(C)C(=O)[C@@H](NC(=O)[C@H](C(C)C)N(C)C(=O)OCc1ccc(O[C@@H]2O[C@H](C(=O)O)[C@@H](O)[C@H](O)[C@H]2O)c(NC(=O)[C@H](CCCNC(N)=O)NC(=O)[C@@H](NC(=O)CCOCCC(=O)NCCNc2ncc(-c3cc(C)cc(F)c3)c(N3CCC(N)CC3)c2-c2nc3ccc(Cl)cc3[nH]2)C(C)C)c1)C(C)C]

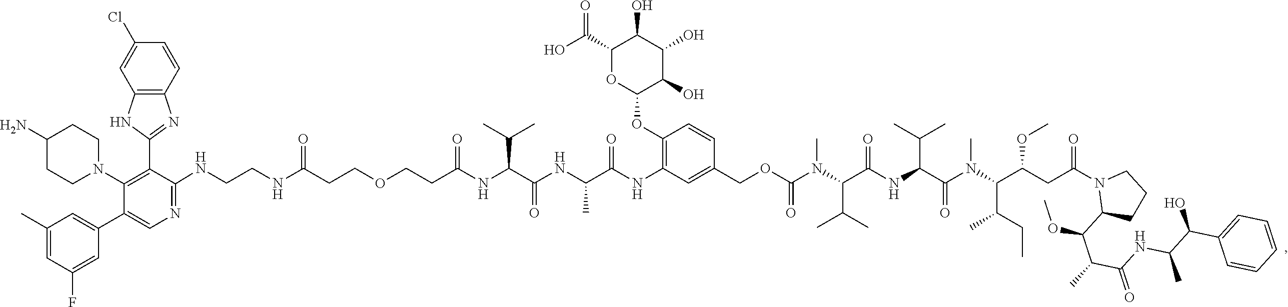 [SMILES: CC[C@H](C)[C@@H]([C@@H](CC(=O)N1CCC[C@H]1[C@H](OC)[C@@H](C)C(=O)N[C@H](C)[C@@H](O)c1ccccc1)OC)N(C)C(=O)[C@@H](NC(=O)[C@H](C(C)C)N(C)C(=O)OCc1ccc(O[C@@H]2O[C@H](C(=O)O)[C@@H](O)[C@H](O)[C@H]2O)c(NC(=O)[C@H](C)NC(=O)[C@@H](NC(=O)CCOCCC(=O)NCCNc2ncc(-c3cc(C)cc(F)c3)c(N3CCC(N)CC3)c2-c2nc3ccc(Cl)cc3[nH]2)C(C)C)c1)C(C)C]

or a pharmaceutically acceptable salt thereof.

### Synthesis of Compounds

Compounds described herein are synthesized using standard synthetic techniques or using methods known in the art in combination with methods described herein.

Unless otherwise indicated, conventional methods of mass spectroscopy, NMR, and HPLC are employed.

Compounds are prepared using standard organic chemistry techniques such as those described in, for example, March's Advanced Organic Chemistry, 6^{th} Edition, John Wiley and Sons, Inc. Alternative reaction conditions for the synthetic transformations described herein may be employed such as variation of solvent, reaction temperature, reaction time, as well as different chemical reagents and other reaction conditions.

In one aspect, compounds described herein are in the form of pharmaceutically acceptable salts.

The term “pharmaceutically acceptable salt” refers to a form of a therapeutically active agent that consists of a cationic form of the therapeutically active agent in combination with a suitable anion, or in alternative embodiments, an anionic form of the therapeutically active agent in combination with a suitable cation. Handbook of Pharmaceutical Salts: Properties. Selection and Use. International Union of Pure and Applied Chemistry, Wiley-VCH 2002. S. M. Berge. L. D. Bighley, D. C. Monkhouse, J. Pharm. Sci. 1977, 66, 1-19.

In some embodiments, pharmaceutically acceptable salts are obtained by reacting a compound of Formula (I) or an NDC disclosed herein with an acid. In some embodiments, the compound of Formula (I)(i.e. free base form) is basic and is reacted with an organic acid or an inorganic acid.

In some embodiments, pharmaceutically acceptable salts are obtained by reacting a compound of Formula (I) or an NDC disclosed herein with a base. In some embodiments, the compound of Formula (I) or NDC disclosed herein is acidic and is reacted with a base. In such situations, an acidic proton of the compound of Formula (I) or an NDC disclosed herein is replaced by a metal ion.

In some embodiments, the compounds of Formula (I) or NDC disclosed herein possess one or more stereocenters and each stereocenter exists independently in either the R or S configuration. In some embodiments, the compound of Formula (I) or an NDC disclosed herein exists in the R configuration. In some embodiments, the compound of Formula (I) or an NDC disclosed herein exists in the S configuration. The compounds presented herein include all diastereomeric, individual enantiomers, atropisomers, and epimeric forms as well as the appropriate mixtures thereof. The compounds and methods provided herein include all cis, trans, syn, anti, entgegen (E), and zusammen (Z) isomers as well as the appropriate mixtures thereof.

Individual stereoisomers are obtained, if desired, by methods such as, stereoselective synthesis and/or the separation of stereoisomers by chiral chromatographic columns or the separation of diastereomers by either non-chiral or chiral chromatographic columns or crystallization and recrystallization in a proper solvent or a mixture of solvents. In certain embodiments, compounds of Formula (I) or the NDCs disclosed herein are prepared as their individual stereoisomers by reacting a racemic mixture of the compound with an optically active resolving agent to form a pair of diastereoisomeric compounds/salts, separating the diastereomers and recovering the optically pure individual enantiomers. In some embodiments, resolution of individual enantiomers is carried out using covalent diastereomeric derivatives of the compounds described herein. In another embodiment, diastereomers are separated by separation/resolution techniques based upon differences in solubility. In other embodiments, separation of stereoisomers is performed by chromatography or by the forming diastereomeric salts and separation by recrystallization, or chromatography, or any combination thereof. Jean Jacques, Andre Collet, Samuel H. Wilen, “Enantiomers, Racemates and Resolutions”, John Wiley And Sons, Inc., 1981. In some embodiments, stereoisomers are obtained by stereoselective synthesis.

### Pharmaceutical Compositions

In some embodiments, the compounds described herein are formulated into pharmaceutical compositions. In some embodiments, the compounds of Formula (I) described herein are formulated into pharmaceutical compositions. In some embodiments, the NDCs described herein are formulated into pharmaceutical compositions.

In some embodiments, the compounds described herein are administered either alone or in combination with pharmaceutically acceptable carriers, excipients or diluents, in a pharmaceutical composition.

### Methods of Treatment

In some embodiments, the methods comprise administering to a subject a therapeutically effective amount of a compound of Formula (I), or an NDC disclosed herein, or a pharmaceutically acceptable salt thereof. In some embodiments, the compound of Formula (I), or an NDC disclosed herein, or pharmaceutically acceptable salt thereof is administered in a pharmaceutical composition. In some embodiments, the subject has cancer. In some embodiments, the cancer is a solid tumor or hematological cancer. In some embodiments, the subject has a noncancerous tumor. In some embodiments, the subject has an adenoma.

In embodiments, the treatment is sufficient to reduce or inhibit the growth of the subject's tumor, reduce the number or size of metastatic lesions, reduce tumor load, reduce primary tumor load, reduce invasiveness, prolong survival time, or maintain or improve the quality of life, or combinations thereof.

In some embodiments, provided herein are methods for killing a tumor cell comprising contacting the tumor cell with a compound of Formula (I), or an NDC disclosed herein, or a pharmaceutically acceptable salt thereof.

In one aspect, provided herein are methods and compositions for treating cancers. Cancer includes tissue and organ carcinogenesis including metastases such as for example gastrointestinal cancer, (e.g., gastric cancer, esophageal cancer, pancreatic cancer colorectal cancer, intestinal cancer, anal cancer, liver cancer, gallbladder cancer, or colon cancer; lung cancer; thyroid cancer; skin cancer (e.g., melanoma); oral cancer; urinary tract cancer (e.g. bladder cancer or kidney cancer); blood cancer (e.g. myeloma or leukemia) or prostate cancer. In some embodiments, the present disclosure provides methods and compositions for treating gastrointestinal cancer in a subject in need thereof by administering an effective amount of a non-peptide targeted therapeutic compound disclosed herein to the subject. Non-limiting examples of gastrointestinal cancers that can be treated according to the methods of the present disclosure include gastric cancer, esophageal cancer, pancreatic cancer, lung cancer (small cell lung cancer and/or non small-cell lung cancer), colorectal cancer, intestinal cancer, anal cancer, liver cancer, gallbladder cancer, or colon cancer. In some embodiments, the cancer is Hodkin's lymphoma or B-cell lymphoma.

In one aspect, provided herein are methods and compositions for treating an adenoma.

In one aspect, provided herein are methods and compositions for treating a peptide hormone G protein-coupled receptor expressing cancer. In some embodiments, the peptide hormone G protein-coupled receptor-expressing cancer to be treated is a primary or metastatic cancer of gastrointestinal origin, such as colorectal cancer, stomach cancer, small intestine cancer, or esophageal cancer. In some embodiments, the peptide hormone G protein-coupled receptor-expressing cancer to be treated is primary or metastatic pancreatic cancer. In some embodiments, the peptide hormone G protein-coupled receptor-expressing cancer to be treated is primary or metastatic lung cancer, such as squamous cell carcinoma, adenosquamous carcinoma, or adenocarcinoma. In some embodiments, the peptide hormone G protein-coupled receptor-expressing cancer to be treated is a sarcoma, such as leiomyosarcoma or rhabdomyosarcoma. In some embodiments, the peptide hormone G protein-coupled receptor-expressing cancer to be treated is a primary or metastasized neuroectodermal tumor, such as aphaechromotcytoma or a paraganglioma. In some embodiments, the peptide hormone G protein-coupled receptor-expressing cancer is a primary or a metastasized bronchopulmonary or a gastrointestinal neuroendocrine tumor. In some embodiments, the cancer is colorectal cancer.

In another aspect, provided herein is a method for treating cancer in a mammal comprising administering to the mammal in need thereof, a non-peptide targeted therapeutic compound disclosed herein. In some embodiments, the cancer expresses one or more peptide hormone G protein-coupled receptors. In some embodiments, the cancer comprises a peptide hormone G protein-coupled receptor-positive cancer. In some embodiments, the cancer comprises a solid tumor. In some embodiments, the cancer comprises a sarcoma, carcinoma, or lymphoma. In some embodiments, the cancer comprises a neuroendocrine tumor. In some embodiments, the cancer comprises an insulinoma In some embodiments, the cancer comprises peptide hormone G protein-coupled receptor-positive (e.g., somatostatin receptor-positive) gastroenteropancreatic neuroendocrine tumors (GEP-NETs).

### Methods of Dosing and Treatment Regimens

In one embodiment, compound of Formula (I), or an NDC disclosed herein, or a pharmaceutically acceptable salt thereof, are used in the preparation of medicaments for the treatment of tumors in a mammal. Methods for treating any of the diseases or conditions described herein in a mammal in need of such treatment, involves administration of pharmaceutical compositions that include at least one compound of Formula (I), or an NDC disclosed herein, or a pharmaceutically acceptable salt thereof in therapeutically effective amounts to said mammal.

In some embodiments, the conjugate or pharmaceutically acceptable salt thereof is administered in combination with another therapeutic agent. In certain embodiments the therapeutic agent is a chemotherapeutic agent. Chemotherapeutic agents include, but are not limited to, lutetium Lu 177 dotatate (LUTATHERA).

### Certain Terminology

Unless otherwise stated, the following terms used in this application have the definitions given below. The use of the term “including” as well as other forms, such as “include”, “includes,” and “included,” is not limiting. The section headings used herein are for organizational purposes only and are not to be construed as limiting the subject matter described.

As used herein. C₁-Cₓ includes C₁-C₂, C₁-C₃ . . . C₁-Cₓ. By way of example only, a group designated as “C₁-C₆” indicates that there are one to six carbon atoms in the moiety, i.e. groups containing 1 carbon atom, 2 carbon atoms, 3 carbon atoms or 4 carbon atoms. Thus, by way of example only, “C₁-C₄ alkyl” indicates that there are one to four carbon atoms in the alkyl group, i.e., the alkyl group is selected from among methyl, ethyl, propyl, iso-propyl, n-butyl, iso-butyl, sec-butyl, and t-butyl.

An “alkyl” group refers to an aliphatic hydrocarbon group. The alkyl group is branched or straight chain. In some embodiments, the “alkyl” group has 1 to 10 carbon atoms, i.e. a C₁-C₁₀alkyl. Whenever it appears herein, a numerical range such as “1 to 10” refers to each integer in the given range; e.g., “1 to 10 carbon atoms” means that the alkyl group consist of 1 carbon atom, 2 carbon atoms, 3 carbon atoms, etc., up to and including 10 carbon atoms, although the present definition also covers the occurrence of the term “alkyl” where no numerical range is designated. In some embodiments, an alkyl is a C₁-C₆alkyl. In one aspect the alkyl is methyl, ethyl, propyl, iso-propyl, n-butyl, iso-butyl, sec-butyl, or t-butyl. Typical alkyl groups include, but are in no way limited to, methyl, ethyl, propyl, isopropyl, butyl, isobutyl, sec-butyl, tertiary butyl, pentyl, neopentyl, or hexyl.

An “alkylene” group refers to a divalent alkyl radical. Any of the above mentioned monovalent alkyl groups may be an alkylene by abstraction of a second hydrogen atom from the alkyl. In some embodiments, an alkylene is a C₁-C₆alkylene. In other embodiments, an alkylene is a C₁-C₄alkylene. Typical alkylene groups include, but are not limited to, —CH₂—, —CH₂CH₂—, —CH₂CH₂CH₂—, —CH₂CH₂CH₂CH₂—, and the like. In some embodiments, an alkylene is —CH₂—.

An “alkoxy” group refers to a (alkyl)O— group, where alkyl is as defined herein.

The term “carbocyclic” or “carbocycle” refers to a ring or ring system where the atoms forming the backbone of the ring are all carbon atoms. The term thus distinguishes carbocyclic from “heterocyclic” rings or “heterocycles” in which the ring backbone contains at least one atom which is different from carbon. In some embodiments, at least one of the two rings of a bicyclic carbocycle is aromatic. In some embodiments, both rings of a bicyclic carbocycle are aromatic. Carbocycles include aryls and cycloalkyls.

As used herein, the term “aryl” refers to an aromatic ring wherein each of the atoms forming the ring is a carbon atom. In one aspect, aryl is phenyl or a naphthyl. In some embodiments, an aryl is a phenyl. In some embodiments, an aryl is a phenyl, naphthyl, indanyl, indenyl, or tetrahydronaphthyl. In some embodiments, an aryl is a C₆-C₁₀aryl. Depending on the structure, an aryl group is a monoradical or a diradical (i.e., an arylene group).

The term “cycloalkyl” refers to a monocyclic or polycyclic aliphatic, non-aromatic radical, wherein each of the atoms forming the ring (i.e. skeletal atoms) is a carbon atom. In some embodiments, cycloalkyls are spirocyclic or bridged compounds. In some embodiments, cycloalkyls are optionally fused with an aromatic ring, and the point of attachment is at a carbon that is not an aromatic ring carbon atom. Cycloalkyl groups include groups having from 3 to 10 ring atoms. In some embodiments, cycloalkyl groups are selected from among cyclopropyl, cyclobutyl, cyclopentyl, cyclopentenyl, cyclohexyl, cyclohexenyl, cycloheptyl, cyclooctyl, spiro[2.2]pentyl, norbornyl and bicycle[1.1.1]pentyl. In some embodiments, a cycloalkyl is a C₃-C₆cycloalkyl. In some embodiments, a cycloalkyl is a C₃-C₄cycloalkyl.

The term “halo” or, alternatively, “halogen” or “halide” means fluoro, chloro, bromo or iodo. In some embodiments, halo is fluoro, chloro, or bromo.

The term “fluoroalkyl” refers to an alkyl in which one or more hydrogen atoms are replaced by a fluorine atom. In one aspect, a fluoroalkyl is a C₁-C₆fluoroalkyl.

The term “heterocycle” or “heterocyclic” refers to heteroaromatic rings (also known as heteroaryls) and heterocycloalkyl rings containing one to four heteroatoms in the ring(s), where each heteroatom in the ring(s) is selected from O, S and N, wherein each heterocyclic group has from 3 to 10 atoms in its ring system, and with the proviso that any ring does not contain two adjacent O or S atoms. Non-aromatic heterocyclic groups (also known as heterocycloalkyls) include rings having 3 to 10 atoms in its ring system and aromatic heterocyclic groups include rings having 5 to 10 atoms in its ring system. The heterocyclic groups include benzo-fused ring systems. Examples of non-aromatic heterocyclic groups are pyrrolidinyl, tetrahydrofuranyl, dihydrofuranyl, tetrahydrothienyl, oxazolidinonyl, tetrahydropyranyl, dihydropyranyl, tetrahydrothiopyranyl, piperidinyl, morpholinyl, thiomorpholinyl, thioxanyl, piperazinyl, aziridinyl, azetidinyl, oxetanyl, thietanyl, homopiperidinyl, oxepanyl, thiepanyl, oxazepinyl, diazepinyl, thiazepinyl, 1,2,3,6-tetrahydropyridinyl, pyrrolin-2-yl, pyrrolin-3-yl, indolinyl, 2H-pyranyl, 4H-pyranyl, dioxanyl, 1,3-dioxolanyl, pyrazolinyl, dithianyl, dithiolanyl, dihydropyranyl, dihydrothienyl, dihydrofuranyl, pyrazolidinyl, imidazolinyl, imidazolidinyl, 3-azabicyclo[3.1.0]hexanyl, 3-azabicyclo[4.1.0]heptanyl, 3H-indolyl, indolin-2-onyl, isoindolin-1-onyl, isoindoline-1,3-dionyl, 3,4-dihydroisoquinolin-1(2H)-onyl, 3,4-dihydroquinolin-2(1H)-onyl, isoindoline-1,3-dithionyl, benzo[d]oxazol-2(3H)-onyl, 1H-benzo[d]imidazol-2(3H)-onyl, benzodithiazol-2(3H)-onyl, and quinolizinyl. Examples of aromatic heterocyclic groups are pyridinyl, imidazolyl, pyrimidinyl, pyrazolyl, triazolyl, pyrazinyl, tetrazolyl, furyl, thienyl, isoxazolyl, thiazolyl, oxazolyl, isothiazolyl, pyrrolyl, quinolinyl, isoquinolinyl, indolyl, benzimidazolyl, benzofuranyl, cinnolinyl, indazolyl, indolizinyl, phthalazinyl, pyridazinyl, triazinyl, isoindolyl, pteridinyl, purinyl, oxadiazolyl, thiadiazolyl, furazanyl, benzofurazanyl, benzothiophenyl, benzothiazolyl, benzoxazolyl, quinazolinyl, quinoxalinyl, naphthyridinyl, and furopyridinyl. The foregoing groups are either C-attached (or C-linked) or N-attached where such is possible. For instance, a group derived from pyrrole includes both pyrrol-1-yl (N-attached) or pyrrol-3-yl (C-attached). Further, a group derived from imidazole includes imidazol-1-yl or imidazol-3-yl (both N-attached) or imidazol-2-yl, imidazol-4-yl or imidazol-5-yl (all C-attached). The heterocyclic groups include benzo-fused ring systems. Non-aromatic heterocycles are optionally substituted with one or two oxo (═O) moieties, such as pyrrolidin-2-one. In some embodiments, at least one of the two rings of a bicyclic heterocycle is aromatic. In some embodiments, both rings of a bicyclic heterocycle are aromatic.

The terms “heteroaryl” or, alternatively, “heteroaromatic” refers to an aryl group that includes one or more ring heteroatoms selected from nitrogen, oxygen and sulfur. Illustrative examples of heteroaryl groups include monocyclic heteroaryls and bicyclic heteroaryls. Monocyclic heteroaryls include pyridinyl, imidazolyk, pyrimidinyl, pyrazolyl, triazolyl, pyrazinyl, tetrazolyl, furyl, thienyl, isoxazolyl, thiazolyl, oxazolyl, isothiazolyl, pyrrolyl, pyridazinyl, triazinyl, oxadiazolyl, thiadiazolyl, and furazanyl. Monocyclic heteroaryls include indolizine, indole, benzofuran, benzothiophene, indazole, benzimidazole, purine, quinolizine, quinoline, isoquinoline, cinnoline, phthalazine, quinazoline, quinoxaline, 1,8-naphthyridine, and pteridine. In some embodiments, a heteroaryl contains 0-4 N atoms in the ring. In some embodiments, a heteroaryl contains 1-4 N atoms in the ring. In some embodiments, a heteroaryl contains 0-4 N atoms, 0-1 O atoms, and 0-1 S atoms in the ring. In some embodiments, a heteroaryl contains 1-4 N atoms, 0-1 O atoms, and 0-1 S atoms in the ring. In some embodiments, heteroaryl is a C₁-C₉heteroaryl. In some embodiments, monocyclic heteroaryl is a C₁-C₅heteroaryl. In some embodiments, monocyclic heteroaryl is a 5-membered or 6-membered heteroaryl. In some embodiments, bicyclic heteroaryl is a C₆-C₉heteroaryl.

A “heterocycloalkyl” group refers to a cycloalkyl group that includes at least one heteroatom selected from nitrogen, oxygen and sulfur. In some embodiments, a heterocycloalkyl is fused with an aryl or heteroaryl. In some embodiments, the heterocycloalkyl is oxazolidinonyl, pyrrolidinyl, tetrahydrofuranyl, tetrahydrothienyl, tetrahydropyranyl, tetrahydrothiopyranyl, piperidinyl, morpholinyl, thiomorpholinyl, piperazinyl, piperidin-2-onyl, pyrrolidine-2,5-dithionyl, pyrrolidine-2,5-dionyl, pyrrolidinonyl, imidazolidinyl, imidazolidin-2-onyl, or thiazolidin-2-onyl. In one aspect, a heterocycloalkyl is a C₂-C₁₀heterocycloalkyl. In another aspect, a heterocycloalkyl is a C₄-C₁₀heterocycloalkyl. In some embodiments, a heterocycloalkyl is monocyclic or bicyclic. In some embodiments, a heterocycloalkyl is monocyclic and is a 3, 4, 5, 6, 7, or 8-membered ring. In some embodiments, a heterocycloalkyl is monocyclic and is a 3, 4, 5, or 6-membered ring. In some embodiments, a heterocycloalkyl is monocyclic and is a 3 or 4-membered ring. In some embodiments, a heterocycloalkyl contains 0-2 N atoms in the ring. In some embodiments, a heterocycloalkyl contains 0-2 N atoms, 0-2 O atoms and 0-1 S atoms in the ring.

The term “bond” or “single bond” refers to a chemical bond between two atoms, or two moieties when the atoms joined by the bond are considered to be part of larger substructure. In one aspect, when a group described herein is a bond, the referenced group is absent thereby allowing a bond to be formed between the remaining identified groups.

The term “moiety” refers to a specific segment or functional group of a molecule. Chemical moieties are often recognized chemical entities embedded in or appended to a molecule.

The term “optionally substituted” or “substituted” means that the referenced group is optionally substituted with one or more additional group(s) individually and independently selected from halogen, —CN, —NH₂, —NH(alkyl), —N(alkyl)₂, —OH, —CO₂H, —CO₂alkyl, —C(═O)NH₂, —C(═O)NH(alkyl), —C(═O)N(alkyl)₂, —S(═O)₂NH₂, —S(═O)₂NH(alkyl), —S(═O)₂N(alkyl)₂, alkyl, cycloalkyl, fluoroalkyl, heteroalkyl, alkoxy, fluoroalkoxy, heterocycloalkyl, aryl, heteroaryl, aryloxy, alkylthio, arylthio, alkylsulfoxide, arylsulfoxide, alkylsulfone, and arylsulfone. In some other embodiments, optional substituents are independently selected from halogen, —CN, —NH₂, —NH(CH₃), —N(CH₃)₂, —OH, —CO₂H, —CO₂(C₁-C₄alkyl), —C(═O)NH₂, —C(═O)NH(C₁-C₄alkyl), —C(═O)N(C₁-C₄alkyl)₂, —S(═O)₂NH₂, —S(═O)₂NH(C₁-C₄alkyl), —S(═O)₂N(C₁-C₄alkyl)₂, C₁-C₄alkyl, C₃-C₆cycloalkyl, C₁-C₄fluoroalkyl, C₁-C₄heteroalkyl, C₁-C₄alkoxy, C₁-C₄fluoroalkoxy, —SC₁-C₄alkyl, —S(═O)C₁-C₄alkyl, and —S(═O)₂C₁-C₄alkyl. In some embodiments, optional substituents are independently selected from halogen, —CN, —NH₂, —OH, —NH(CH₃), —N(CH₃)₂, —CH₃, —CH₂CH₃, —CHF₂, —CF₃, —OCH₃, —OCHF₂, and —OCF₃. In some embodiments, substituted groups are substituted with one or two of the preceding groups. In some embodiments, an optional substituent on an aliphatic carbon atom (acyclic or cyclic) includes oxo (═O).

The term “modulate” as used herein, means to interact with a target either directly or indirectly so as to alter the activity of the target, including, by way of example only, to enhance the activity of the target, to inhibit the activity of the target, to limit the activity of the target, or to extend the activity of the target.

The term “modulator” as used herein, refers to a molecule that interacts with a target either directly or indirectly. The interactions include, but are not limited to, the interactions of an agonist, partial agonist, an inverse agonist, antagonist, degrader, or combinations thereof. In some embodiments, a modulator is an agonist.

The terms “administer,” “administering”, “administration,” and the like, as used herein, refer to the methods that may be used to enable delivery of compounds or compositions to the desired site of biological action. These methods include, but are not limited to oral routes, intraduodenal routes, parenteral injection (including intravenous, subcutaneous, intraperitoneal, intramuscular, intravascular or infusion). Those of skill in the art are familiar with administration techniques that can be employed with the compounds and methods described herein.

The terms “co-administration” or the like, as used herein, are meant to encompass administration of the selected therapeutic agents to a single patient, and are intended to include treatment regimens in which the agents are administered by the same or different route of administration or at the same or different time.

The terms “effective amount” or “therapeutically effective amount,” as used herein, refer to a sufficient amount of an agent or a compound being administered, which will relieve to some extent one or more of the symptoms of the disease or condition being treated. The result includes reduction and/or alleviation of the signs, symptoms, or causes of a disease, or any other desired alteration of a biological system. For example, an “effective amount” for therapeutic uses is the amount of the composition comprising a compound as disclosed herein required to provide a clinically significant decrease in disease symptoms. An appropriate “effective” amount in any individual case is optionally determined using techniques, such as a dose escalation study.

The terms “enhance” or “enhancing,” as used herein, means to increase or prolong either in potency or duration a desired effect. Thus, in regard to enhancing the effect of therapeutic agents, the term “enhancing” refers to the ability to increase or prolong, either in potency or duration, the effect of other therapeutic agents on a system. An “enhancing-effective amount,” as used herein, refers to an amount adequate to enhance the effect of another therapeutic agent in a desired system.

The term “pharmaceutical combination” as used herein, means a product that results from the mixing or combining of more than one active ingredient and includes both fixed and non-fixed combinations of the active ingredients. The term “fixed combination” means that the active ingredients, e.g. a compound of Formula (I), or a pharmaceutically acceptable salt thereof, and a co-agent, are both administered to a patient simultaneously in the form of a single entity or dosage. The term “non-fixed combination” means that the active ingredients, e.g. a compound of Formula (I), or a pharmaceutically acceptable salt thereof, and a co-agent, are administered to a patient as separate entities either simultaneously, concurrently or sequentially with no specific intervening time limits, wherein such administration provides effective levels of the two compounds in the body of the patient. The latter also applies to cocktail therapy, e.g, the administration of three or more active ingredients.

The terms “article of manufacture” and “kit” are used as synonyms.

The term “subject” or “patient” encompasses mammals. Examples of mammals include, but are not limited to, any member of the Mammalian class: humans, non-human primates such as chimpanzees, and other apes and monkey species; farm animals such as cattle, horses, sheep, goats, swine; domestic animals such as rabbits, dogs, and cats; laboratory animals including rodents, such as rats, mice and guinea pigs, and the like. In one aspect, the mammal is a human.

The terms “treat,” “treating” or “treatment,” as used herein, include alleviating, abating or ameliorating at least one symptom of a disease or condition, preventing additional symptoms, inhibiting the disease or condition, e.g., arresting the development of the disease or condition, relieving the disease or condition, causing regression of the disease or condition, relieving a condition caused by the disease or condition, or stopping the symptoms of the disease or condition either prophylactically and/or therapeutically.

### EXAMPLES

The following examples are provided for illustrative purposes only and not to limit the scope of the claims provided herein.

### Synthesis of Compounds

### Example A. (S)-4-[N—(S)-1-[N—(S)-1-[N-4-({[(S)-1-[N—(S)-1-{[(1S,2R)-4-[(S)-2-{(1R,2R)-2-[N-(1R,2S)-2-hydroxy-1-methyl-2-phenylethylcarbamoyl]-1-methoxypropyl}-1-pyrrolidinyl]-1-[(S)-1-methylpropyl]-2-methoxy-4-oxobutyl]-N-methylcarbamoyl}-2-methylpropylcarbamoyl]-2-methylpropyl](methyl)(aminocarbonyloxy)}methyl)-2-(2-aminoethoxy)phenylcarbamoyl]-4-ureidobutylcarbamoyl]-2-methylpropylcarbamoyl]-4-[3-(2-{N-2-[4-(4-amino-1-piperidyl)-3-(6-chloro-1H-1,3-benzimidazol-2-yl)-5-(3-fluoro-5-tolyl)-2-pyridylamino]ethylcarbamoyl}ethoxy)propionylamino]butyric acid (Compound 1)

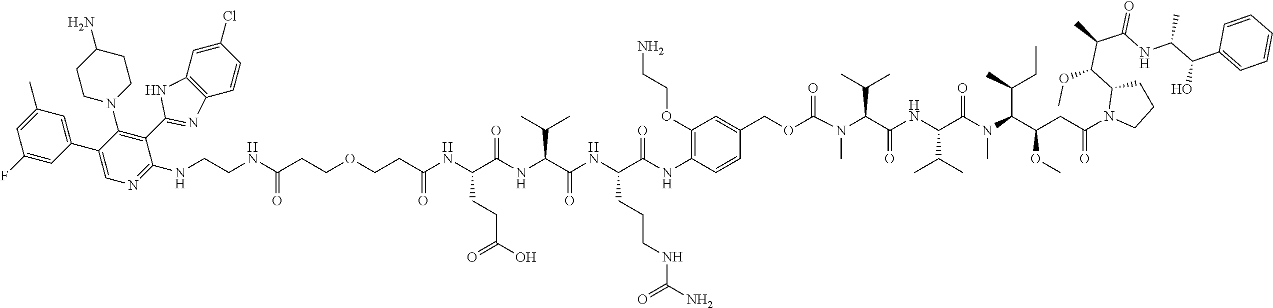 [SMILES: CC[C@H](C)[C@@H]([C@@H](CC(=O)N1CCC[C@H]1[C@H](OC)[C@@H](C)C(=O)N[C@H](C)[C@@H](O)c1ccccc1)OC)N(C)C(=O)[C@@H](NC(=O)[C@H](C(C)C)N(C)C(=O)OCc1ccc(NC(=O)[C@H](CCCNC(N)=O)NC(=O)[C@@H](NC(=O)[C@H](CCC(=O)O)NC(=O)CCOCCC(=O)NCCNc2ncc(-c3cc(C)cc(F)c3)c(N3CCC(N)CC3)c2-c2nc3ccc(Cl)cc3[nH]2)C(C)C)c(OCCN)c1)C(C)C]

Step A-1, preparation of tert-butyl (2-(5-formyl-2-nitrophenoxy)ethyl)carbamate: Into a 40-mL round-bottom flask was placed a mixture of 3-hydroxy-4-nitrobenzaldehyde (1 g, 1 Eq, 6 mmol), tert-butyl (2-bromoethyl)carbamate (3 g, 2 Eq, 0.01 mol), potassium carbonate (2.2 g, 0.93 mL, 3 Eq, 16 mmol) and acetonitrile (20 mL). The reaction mixture was stirred at 80° C. for 3 hours. The mixture was concentrated under reduced pressure. The crude product was purified by medium pressure liquid chromatography (MPLC) with the following conditions: Silica gel column 120 g, petroleum ether (PE)/ethyl acetate (EtOAc) system, the ratio of EtOAc from 0% to 85% in 30 minutes, Flow rate: 60 mL/min; Wave Length: 254 nm. The collected fractions were concentrated under reduced pressure to provide tert-butyl (2-(5-formyl-2-nitrophenoxy)ethyl)carbamate (350 mg, 1.13 mmol, 20%); (M+H)⁺=333.1.

Step A-2, preparation of tert-butyl (2-(2-amino-5-(hydroxymethyl)phenoxy)ethyl)carbamate: Into a 250-mL round-bottom flask purged and maintained with an inert atmosphere of nitrogen was placed tert-butyl (2-(5-formyl-2-nitrophenoxy)ethyl)carbamate (350 mg, 1 Eq, 1.13 mmol), EtOAc (50 mL) and triethylamine (114 mg, 157 μL, 0.999 Eq, 1.13 mmol), and Pearlman's catalyst (60 mg, 0.50 Eq, 0.56 mmol). The flask was evacuated and flushed with hydrogen three times, followed by flushing with hydrogen. The mixture was stirred at 25° C. for 24 hours under the pressure of H₂. The reaction mixture was filtered through a diatomite cushion and the filtrate was concentrated under reduced pressure to provide tert-butyl (2-(2-amino-5-(hydroxymethyl)phenoxy)ethyl)carbamate (300 mg, 1.06 mmol, 94.2%); (M+H)⁺=283.1.

Step A-3, preparation of tert-butyl (2-(2-((S)-2-((S)-2-((((9H-fluoren-9-yl)methoxy)carbonyl)amino)-3-methylbutanamido)-5-ureidopentanamido)-5-(hydroxymethyl)phenoxy)ethyl)carbamate: Into a 40-mL vial was placed tert-butyl (2-(2-amino-5-(hydroxymethyl)phenoxy)ethyl)carbamate (300 mg, 1 Eq, 1.06 mmol) and (S)-2-((S)-2-((((9H-fluoren-9-yl)methoxy)carbonyl)amino)-3-methylbutanamido)-5-ureidopentanoic acid (790 mg, 1.50 Eq, 1.59 mmol) in dichloromethane (DCM) (6 mL) and ethyl 2-ethoxyquinoline-1(2H)-carboxylate (526 mg, 2.00 Eq, 2.13 mmol) at room temperature. The resulting reaction mixture was stirred for 24 hours at 25° C. The crude product was purified by MPLC with the following conditions: Silica gel column 120 g. DCM/methanol (MeOH) system, the ratio of MeOH from 0% to 20% in 40 min, Flow rate: 60 mL/min; Wave Length: 254 nm. The collected fractions were concentrated under reduced pressure to provide tert-butyl (2-(2-((S)-2-((S)-2-((((9H-fluoren-9-yl)methoxy)carbonyl)amino)-3-methylbutanamido)-5-ureidopentanamido)-5-(hydroxymethyl)phenoxy)ethyl)carbamate (400 mg, 526 μmol, 49.5%); (M+H)⁺=761.3.

Step A-4, preparation tert-butyl (2-(2-((S)-2-((S)-2-((((9H-fluoren-9-yl)methoxy)carbonyl)amino)-3-methylbutanamido)-5-ureidopentanamido)-5-((((4-nitrophenoxy)carbonyl)oxy)methyl)phenoxy)ethyl)carbamate: Into a 40-mL vial was placed tert-butyl (2-(2-((S)-2-((S)-2-((((9H-fluoren-9-yl)methoxy)carbonyl)amino)-3-methylbutanamido)-5-ureidopentanamido)-5-(hydroxymethyl)phenoxy)ethyl)carbamate (400 mg, 1 Eq, 526 μmol), carbonicacid,bis(4-nitrophenyl)ester (240 mg, 1.50 Eq. 789 μmol) and diisopropylethylamine (204 mg, 0.27 mL, 3.00 Eq. 1.58 mmol) in dimethylformamide (DMF) (4 mL) at room temperature. The resulting reaction mixture was stirred for 3 hours at 25° C. The reaction mixture was concentrated under reduced pressure. The crude product was purified by MPLC with the following conditions: Silica gel column 120 g, DCM/MeOH system, the ratio of MeOH from 0% to 20% in 30 min, Flow rate: 60 mL/min; Wave Length: 254 nm. The collected fractions were concentrated under reduced pressure to provide tert-butyl (2-(2-((S)-2-((S)-2-((((9H-fluoren-9-yl)methoxy)carbonyl)amino)-3-methylbutanamido)-5-ureidopentanamido)-5-((((4-nitrophenoxy)carbonyl)oxy)methyl)phenoxy)ethyl)carbamate (400 mg, 432 μmol, 82.2%); (M+H)⁺=927.6.

Step A-5, preparation of 4-((S)-2-((S)-2-((((9H-fluoren-9-yl)methoxy)carbonyl)amino)-3-methylbutanamido)-5-ureidopentanamido)-3-(2-((tert-butoxycarbonyl)amino)ethoxy)benzyl((S)-1-(((S)-1-(((3R,4S,5S)-1-((S)-2-((1R,2R)-3-(((1S,2R)-1-hydroxy-1-phenylpropan-2-yl)amino)-1-methoxy-2-methyl-3-oxopropyl)pyrrolidin-1-yl)-3-methoxy-5-methyl-1-oxoheptan-4-yl)(methyl)amino)-3-methyl-1-oxobutan-2-yl)amino)-3-methyl-1-oxobutan-2-yl)(methyl)carbamate: Into a 8-mL vial was placed a mixture of tert-butyl (2-(2-((S)-2-((S)-2-((((9H-fluoren-9-yl)methoxy)carbonyl)amino)-3-methylbutanamido)-5-ureidopentanamido)-5-((((4-nitrophenoxy)carbonyl)oxy)methyl)phenoxy)ethyl)carbamate (160 mg, 1 Eq, 173 μmol), (S)—N-((3R,4S,5S)-1-((S)-2-((1R,2R)-3-(((1S,2R)-1-hydroxy-1-phenylpropan-2-yl)amino)-1-methoxy-2-methyl-3-oxopropyl)pyrrolidin-1-yl)-3-methoxy-5-methyl-1-oxoheptan-4-yl)-N,3-dimethyl-2-((S)-3-methyl-2-(methylamino)butanamido)butanamide (100 mg, 0.806 Eq, 139 μmol), 1-Hydroxy-1H-benzotriazole (35 mg, 36 μL, 1.5 Eq, 0.26 mmol), diisopropylethylamine (67 mg, 89 μL, 3.0 Eq, 0.52 mmol), and DMF (2 mL). The reaction mixture was stirred at 25° C. for 3 hours to provide 4-((S)-2-((S)-2-((((9H-fluoren-9-yl)methoxy)carbonyl)amino)-3-methylbutanamido)-5-ureidopentanamido)-3-(2-((tert-butoxycarbonyl)amino)ethoxy)benzyl((S)-1-(((S)-1-(((3R,4S,5S)-1-((S)-2-((1R,2R)-3-(((S,2R)-1-hydroxy-1-phenylpropan-2-yl)amino)-1-methoxy-2-methyl-3-oxopropyl)pyrrolidin-1-yl)-3-methoxy-5-methyl-1-oxoheptan-4-yl)methyl)amino)-3-methyl-1-oxobutan-2-yl)amino)-3-methyl-1-oxobutan-2-yl)methyl)carbamate (170 mg, 113 μmol, 65.4%); (M+H)⁺=1506.2. The reaction system was not purified and was directly put into the next step.

Step A-6, preparation of 4-((S)-2-((S)-2-amino-3-methylbutanamido)-5-ureidopentanamido)-3-(2-((tert-butoxycarbonyl)amino)ethoxy)benzyl ((S)-1-(((S)-1-(((3R,4S,5S)-1-((S)-2-((1R,2R)-3-(((1S,2R)-1-hydroxy-1-phenylpropan-2-yl)amino)-1-methoxy-2-methyl-3-oxopropyl)pyrrolidin-1-yl)-3-methoxy-5-methyl-1-oxoheptan-4-yl)(methyl)amino)-3-methyl-1-oxobutan-2-yl)amino)-3-methyl-1-oxobutan-2-yl)(methyl)carbamate: Into a 8-mL vial was placed 4-((S)-2-((S)-2-((((9H-fluoren-9-yl)methoxy)carbonyl)amino)-3-methylbutanamido)-5-ureidopentanamido)-3-(2-((tert-butoxy carbonyl)amino)ethoxy)benzyl ((S)-1-(((S)-1-(((3R,4S,5S)-1-((S)-2-((1R,2R)-3-(((1S,2R)-1-hydroxy-1-phenylpropan-2-yl)amino)-1-methoxy-2-methyl-3-oxopropyl)pyrrolidin-1-yl)-3-methoxy-5-methyl-1-oxoheptan-4-yl)(methyl)amino)-3-methyl-1-oxobutan-2-yl)amino)-3-methyl-1-oxobutan-2-yl)(methyl)carbamate (170 mg, 1 Eq, 113 μmol) in DMF (2 mL), and 1,8-Diazabicyclo[5.4.0]undec-7-ene (86 mg, 84 μL, 5.0 Eq, 0.56 mmol). The reaction mixture was stirred at 25° C. for 30 minutes. The mixture was directly purified by MPLC with the following conditions: Column, WelFlash™, C 18120 g, Spherical 20-40 μm; Mobile phase, Water (0.1% trifluoroacetic acid (TFA)) and acetonitrile (CAN) (5% ACN to 5% ACN in 1 min, 30% ACN up to 80% in 8 min, 95% ACN to 95% in 3 min); Total flow rate, 70 mL/min; Detector, UV 220 nm. This provided 4-((S)-2-((S)-2-amino-3-methylbutanamido)-5-ureidopentanamido)-3-(2-((tert-butoxycarbonyl)amino)ethoxy)benzyl ((S)-1-(((S)-1-(((3R,4S,5S)-1-((S)-2-((1R,2R)-3-(((I S,2R)-1-hydroxy-1-phenylpropan-2-yl)amino)-1-methoxy-2-methyl-3-oxopropyl)pyrrolidin-1-yl)-3-methoxy-5-methyl-1-oxoheptan-4-yl)(methyl)amino)-3-methyl-1-oxobutan-2-yl)amino)-3-methyl-1-oxobutan-2-yl)(methyl)carbamate (120 mg, 93.6 μmol, 82.8%); (M+H)⁺=1282.9.

Step A-7, preparation of tert-butyl (S)-4-((((9H-fluoren-9-yl)methoxy)carbonyl)amino)-5-(((S)-1-(((S)-1-((2-(2-((tert-butoxycarbonyl)amino)ethoxy)-4-((5S,8S,11S,12R)-11-((S)-sec-butyl)-12-(2-((S)-2-((1R,2R)-3-((1S,2R)-1-hydroxy-1-phenylpropan-2-yl)amino)-1-methoxy-2-methyl-3-oxopropyl)pyrrolidin-1-yl)-2-oxoethyl)-5,8-diisopropyl-4,10-dimethyl-3,6,9-trioxo-2,13-dioxa-4,7,10-triazatetradecyl)phenyl)amino)-1-oxo-5-ureidopentan-2-yl)amino)-3-methyl-1-oxobutan-2-yl)amino)-5-oxopentanoate: Into a 40-mL vial was placed a mixture of 4-((S)-2-((S)-2-amino-3-methylbutanamido)-5-ureidopentanamido)-3-(2-((tert-butoxycarbonyl)amino)ethoxy)benzyl ((S)-1-(((S)-1-(((3R,4S,5S)-1-((S)-2-((1R,2R)-3-(((1S,2R)-1-hydroxy-1-phenylpropan-2-yl)amino)-1-methoxy-2-methyl-3-oxopropyl)pyrrolidin-1-yl)-3-methoxy-5-methyl-1-oxoheptan-4-yl)(methyl)amino)-3-methyl-1-oxobutan-2-yl)amino)-3-methyl-1-oxobutan-2-yl)methyl)carbamate (60 mg, 1 Eq, 47 μmol), (S)-2-((((9H-fluoren-9-yl)methoxy)carbonyl)amino)-5-(tert-butoxy)-5-oxopentanoic acid (35 mg, 1.8 Eq, 82 μmol), diisopropylethylamine (DIEA) (18 mg, 24 μL, 3.0 Eq, 0.14 mmol), HATU (36 mg, 2.0 Eq, 95 μmol), and DMF (1.2 mL). The reaction mixture was stirred at 25° C. for 1 hour. The mixture did not need to be purified, and the crude product was used directly in the next step; (M+H)⁺=1648.0.

Step A-8, preparation of tert-butyl (S)-4-amino-5-((S)-1-(((S)-1-((2-(2-((tert-butoxy carbonyl)amino)ethoxy)-4-((5S,8S,11S,12R)-11-((S)-sec-butyl)-12-(2-((S)-2-((1R,2R)-3-(((1S,2R)-1-hydroxy-1-phenylpropan-2-yl)amino)-1-methoxy-2-methyl-3-oxopropyl)pyrrolidin-1-yl)-2-oxoethyl)-5,8-diisopropyl-4,10-dimethyl-3,6,9-trioxo-2,13-dioxa-4,7,10-triazatetradecyl)phenyl)amino)-1-oxo-5-ureidopentan-2-yl)amino)-3-methyl-1-oxobutan-2-yl)amino)-5-oxopentanoate: Into a 8-mL vial was placed tert-butyl (S)-4-((((9H-fluoren-9-yl)methoxy)carbonyl)amino)-5-(((S)-1-(((S)-1-((2-(2-((tert-butoxycarbonyl)amino)ethoxy)-4-((5S,8S,11S,12R)-11-((S)-sec-butyl)-12-(2-((S)-2-((1R,2R)-3-(((1S,2R)-1-hydroxy-1-phenylpropan-2-yl)amino)-1-methoxy-2-methyl-3-oxopropyl)pyrrolidin-1-yl)-2-oxoethyl)-5,8-diisopropyl-4,10-dimethyl-3,6,9-trioxo-2,13-dioxa-4,7,10-triazatetradecyl)phenyl)amino)-1-oxo-5-ureidopentan-2-yl)amino)-3-methyl-1-oxobutan-2-yl)amino)-5-oxopentanoate (40 mg, 1 Eq, 24 μmol), 2,3,4,6,7,8,9,10-octahydropyrimido[1,2-a]azepine (10 mg, 2.8 Eq, 66 μmol), and DMF. The reaction mixture was stirred at 25° C. for 30 minutes. The mixture was directly purified by MPLC with the following conditions: Column, WelFlash™, C18 120 g, Spherical 20-40 μm; Mobile phase, Water (0.1% TFA) and ACN (5% ACN to 5% ACN in 1 min, 30% ACN up to 80% in 8 min, 95% ACN to 95% in 3 min); Total flow rate, 70 mL/min; Detector, UV 220 nm. This provided tert-butyl (S)-4-amino-5-(((S)-1-(((S)-1-((2-(2-((tert-butoxy carbonyl)amino)ethoxy)-4-((5S,8S,11S,12R)-11-((S)-sec-butyl)-12-(2-((S)-2-((1R,2R)-3-(((1S,2R)-1-hydroxy-1-phenylpropan-2-yl)amino)-1-methoxy-2-methyl-3-oxopropyl)pyrrolidin-1-yl)-2-oxoethyl)-5,8-diisopropyl-4,10-dimethyl-3,6,9-trioxo-2,13-dioxa-4,7,10-triazatetradecyl)phenyl)amino)-1-oxo-5-ureidopentan-2-yl)amino)-3-methyl-1-oxobutan-2-yl)amino)-5-oxopentanoate (40 mg, 27 μmol); (M/2H)⁺=734.4.

Step A-9, preparation of tert-butyl (S)-5-(((S)-1-(((S)-1-((2-(2-((tert-butoxycarbonyl)amino)ethoxy)-4-((5S,8S,11S,12R)-11-((S)-sec-butyl)-12-(2-((S)-2-((1R,2R)-3-(((1S,2R)-1-hydroxy-1-phenylpropan-2-yl)amino)-1-methoxy-2-methyl-3-oxopropyl)pyrrolidin-1-yl)-2-oxoethyl)-5,8-diisopropyl-4,10-dimethyl-3,6,9-trioxo-2,13-dioxa-4,7,10-triazatetradecyl)phenyl)amino)-1-oxo-5-ureidopentan-2-yl)amino)-3-methyl-1-oxobutan-2-yl)amino)-4-(3-(3-((2-((4-(4-((tert-butoxycarbonyl)amino)piperidin-1-yl)-3-(6-chloro-1H-benzo[d]imidazol-2-yl)-5-(3-fluoro-5-methylphenyl)pyridin-2-yl)amino)ethyl)amino)-3-oxopropoxy)propanamido)-5-oxopentanoate: Into a 8-mL vial was placed tert-butyl (S)-4-amino-5-(((S)-1-(((S)-1-((2-(2-((tert-butoxycarbonyl)amino)ethoxy)-4-((5S,8S,11S,12R)-11-((S)-sec-butyl)-12-(2-((S)-2-((1R,2R)-3-(((1S,2R)-1-hydroxy-1-phenylpropan-2-yl)amino)-1-methoxy-2-methyl-3-oxopropyl)pyrrolidin-1-yl)-2-oxoethyl)-5,8-diisopropyl-4,10-dimethyl-3,6,9-trioxo-2,13-dioxa-4,7,10-triazatetradecyl)phenyl)amino)-1-oxo-5-ureidopentan-2-yl)amino)-3-methyl-1-oxobutan-2-yl)amino)-5-oxopentanoate (40 mg, 1 Eq, 27 μmol), 3-(3-((2-((4-(4-((tert-butoxy carbonyl)amino)piperidin-1-yl)-3-(6-chloro-1H-benzo[d]imidazol-2-yl)-5-(3-fluoro-5-methylphenyl)pyridin-2-yl)amino)ethyl)amino)-3-oxopropoxy)propanoic acid (20 mg, 0.99 Eq, 27 μmol), pentafluorophenyldiphenylphosphinate (25 mg, 2.4 Eq, 65 μmol) and DMF (0.8 mL) then 4-methylmorfolin (10 mg, 11 μL, 3.6 Eq, 99 μmol) was added and the reaction mixture was stirred at 25° C. for an additional hour. The mixture was directly purified by MPLC with the following conditions: Column, WelFlash™, C18120 g, Spherical 20-40 μm; Mobile phase, Water (0.1% TFA) and ACN (5% ACN to 5% ACN in 1 min, 30% ACN up to 80% in 8 min, 95% ACN to 95% in 3 min); Total flow rate, 70 mL/min; Detector, UV 220 nm. This resulted in tert-butyl (S)-5-(((S)-1-(((S)-1-((2-(2-((tert-butoxycarbonyl)amino)ethoxy)-4-((5S,8S,1S,12R)-11-((S)-sec-butyl)-12-(2-((S)-2-((1R,2R)-3-(((1S,2R)-1-hydroxy-1-phenylpropan-2-yl)amino)-1-methoxy-2-methyl-3-oxopropyl)pyrrolidin-1-yl)-2-oxoethyl)-5,8-diisopropyl-4,10-dimethyl-3,6,9-trioxo-2,13-dioxa-4,7,10-triazatetradecyl)phenyl)amino)-1-oxo-5-ureidopentan-2-yl)amino)-3-methyl-1-oxobutan-2-yl)amino)-4-(3-(3-((2-((4-(4-((tert-butoxy carbonyl)amino)piperidin-1-yl)-3-(6-chloro-1H-benzo[d]imidazol-2-yl)-5-(3-fluoro-5-methylphenyl)pyridin-2-yl)amino)ethyl)amino)-3-oxopropoxy)propanamido)-5-oxopentanoate (30 mg, 14 μmol, 50%); (M/2+H)⁺=1094.0.

Step A-10. Preparation of (S)-4-[N—(S)-1-[N—(S)-1-[N-4-({[(S)-1-[N—(S)-1-{[(1S,2R)-4-[(S)-2-{(1R,2R)-2-[N-(1R,2S)-2-hydroxy-1-methyl-2-phenylethylcarbamoyl]-1-methoxypropyl}-1-pyrrolidinyl]-1-[(S)-1-methylpropyl]-2-methoxy-4-oxobutyl]-N-methylcarbamoyl}-2-methylpropylcarbamoyl]-2-methlypropyl](methyl)(aminocarbonyloxy)}methyl)-2-(2-aminoethoxy)phenylcarbamoyl]-4-ureidobutylcarbamoyl]-2-methylpropylcarbamoyl]-4-[3-(2-{N-2-[4-(4-amino-1-piperidyl)-3-(6-chloro-1H-1,3-benzimidazol-2-yl)-5-(3-fluoro-5-tolyl)-2-pyridylamino]ethylcarbamoyl}ethoxy)propionylamino]butyric acid: Into a 8 mL vial was placed a mixture of tert-butyl (S)-5-(((S)-1-(((S)-4-((2-(2-aminoethoxy)-4-((5S,8S,11S,12R)-11-((S)-sec-butyl)-12-(2-((S)-2-((1R,2R)-3-(((1S,2R)-1-hydroxy-1-phenylpropan-2-yl)amino)-1-methoxy-2-methyl-3-oxopropyl)pyrrolidin-1-yl)-2-oxoethyl)-5,8-diisopropyl-4,10-dimethyl-3,6,9-trioxo-2,13-dioxa-4,7,10-triazatetradecyl)phenyl)amino)-1-oxo-5-ureidopentan-2-yl)amino)-3-methyl-1-oxobutan-2-yl)amino)-4-(3-(3-((2-((4-(4-((tert-butoxycarbonyl)amino)piperidin-1-yl)-3-(6-chloro-1H-benzo[d]imidazol-2-yl)-5-(3-fluoro-5-methylphenyl)pyridin-2-yl)amino)ethyl)amino)-3-oxopropoxy)propanamido)-5-oxopentanoate (30 mg, 1 Eq, 14 μmol) and TFA/DCM (0.6 ml), and the reaction mixture was stirred at 0° C. for 30 minutes. The crude product was purified by Prep-high pressure liquid chromatography (HPLC) with the following conditions: Column: SunFire prep OBD 19*150 mm 5 um; Mobile Phase A: Water (0.1% TFA); Mobile Phase B: ACN; Gradient: 28% B to 55% B in 14 min; Flow rate: 20 mL/min; Wave Length: 220 nm. The collected fractions were dried by lyophilization to provide (S)-5-(((S)-1-(((S)-1-((2-(2-aminoethoxy)-4-((5S,8S,11S,12R)-11-((S)-sec-butyl)-12-(2-((S)-2-((1R,2R)-3-(((1S,2R)-1-hydroxy-1-phenylpropan-2-yl)amino)-1-methoxy-2-methyl-3-oxopropyl)pyrrolidin-1-yl)-2-oxoethyl)-5,8-diisopropyl-4,10-dimethyl-3,6,9-trioxo-2,13-dioxa-4,7,10-triazatetradecyl)phenyl)amino)-1-oxo-5-ureidopentan-2-yl)amino)-3-methyl-1-oxobutan-2-yl)amino)-4-(3-(3-((2-((4-(4-aminopiperidin-1-yl)-3-(6-chloro-1H-benzo[d]imidazol-2-yl)-5-(3-fluoro-5-methylphenyl)pyridin-2-yl)amino)ethyl)amino)-3-oxopropoxy)propanamido)-5-oxopentanoic acid-2,2,2-trifluoroacetic acid (1/3) (11.7 mg, 5.15 μmol, 36%); (M+H)⁺=2273.0.

The following conjugates were prepared similarly to Example A with appropriate substituting reagents and substrates at different steps and they may require additional functional group modifications via well-known chemistry with appropriate reagents.

| | MS |
|---|---|
| Compound | (M + H)⁺ |
| (S)-4-[N-(S)-1-[N-(S)-1-[N-4-({[(S)-1-[N-(S)-1-{[(1S,2R)-4-[(S)-2-{(1R,2R)-2-[N-(1R,2S)-2-hydroxy-1-methyl-2- | 1939.9 |
| phenylethylcarbamoyl]-1-methoxypropyl}-1-pyrrolidinyl]-1-[(S)-1-methylpropyl]-2-methoxy-4-oxobutyl]-N- | |
| methylcarbamoyl}-2-methylpropylcarbamoyl]-2-methylpropyl](methyl)(aminocarbonyloxy)}methyl)-3- | |
| (trifluoromethyl)phenylcarbamoyl]-4-ureidobutylcarbamoyl]-2-methylpropylcarbamoyl]-4-[3-(2-{N-2-[4-(4-amino-1-piperidyl)- | |
| 3-(6-chloro-1H-1,3-benzimidazol-2-yl)-5-(3-fluoro-5-tolyl)-2-pyridylamino]ethylcarbamoyl}ethoxy)propionylamino]butyric | |
| acid | |
| 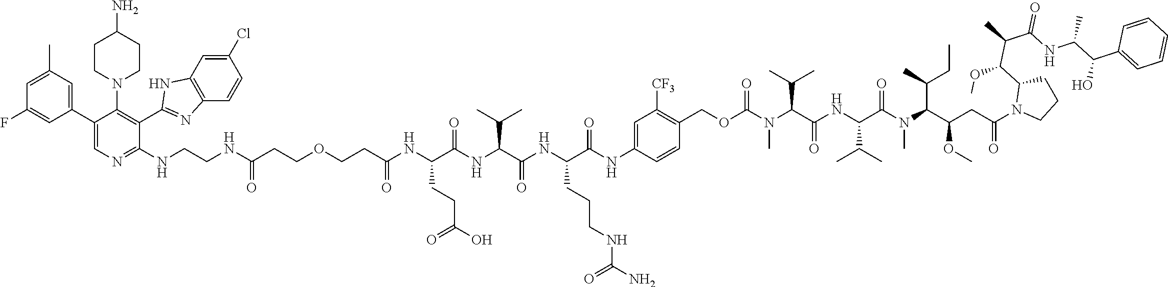 [SMILES: CC[C@H](C)[C@@H]([C@@H](CC(=O)N1CCC[C@H]1[C@H](OC)[C@@H](C)C(=O)N[C@H](C)[C@@H](O)c1ccccc1)OC)N(C)C(=O)[C@@H](NC(=O)[C@H](C(C)C)N(C)C(=O)OCc1ccc(NC(=O)[C@H](CCCNC(N)=O)NC(=O)[C@@H](NC(=O)[C@H](CCC(=O)O)NC(=O)CCOCCC(=O)NCCNc2ncc(-c3cc(C)cc(F)c3)c(N3CCC(N)CC3)c2-c2nc3ccc(Cl)cc3[nH]2)C(C)C)cc1C(F)(F)F)C(C)C] | |
| (Compound 2) | |
| 2-({[(S)-1-[N-(S)-1-{[(1S,2R)-4-[(S)-2-{(1R,2R)-2-[N-(1R,2S)-2-hydroxy-1-methyl-2-phenylethylcarbamoyl]-1- | 2047.0 |
| methoxypropyl}-1-pyrrolidinyl]-1-[(S)-1-methylpropyl]-2-methoxy-4-oxobutyl]-N-methylcarbamoyl}-2- | |
| methylpropylcarbamoyl]-2-methylpropyl](methyl)(aminocarbonyloxy)}methyl)-5-[(S)-2-[(S)-2[(S)-2-(14-{N-2-[4-(4-amino-1- | |
| piperidyl)-3-(6-chloro-1H-1,3-benzimidazol-2-yl)-5-(3-fluoro-5-tolyl)-2-pyridylamino]ethylcarbamoyl}-3,6,9,12- | |
| tetraoxatetradecylcarbonylamino)-4-carbamoylbutyrylamino]-3-methylbutyrylamino]-5-ureidovalerylamino]benzoic acid | |
| 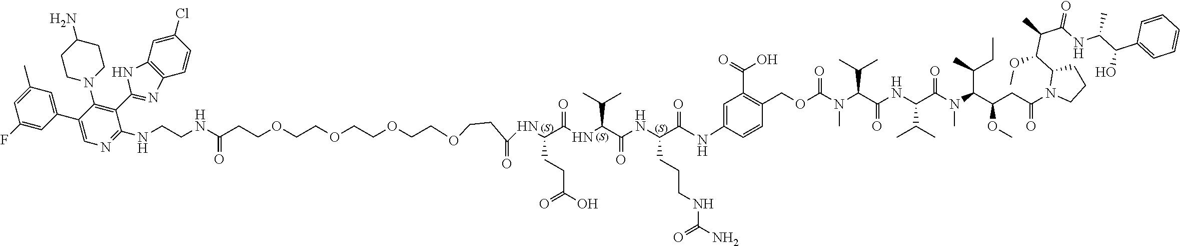 | |
| (Compound 3) | |
| 2-({[(S)-1-[N-(S)-1-{[(1S,2R)-4-[(S)-2-{(1R,2R)-2-[N-(1R,2S)-2-hydroxy-1-methyl-2-phenylethylcarbamoyl]-1- | 1919.0 |
| methoxypropyl}-1-pyrrolidinyl]-1-[(S)-1-methylpropyl]-2-methoxy-4-oxobutyl]-N-methylcarbamoyl}-2- | |
| methylpropylcarbamoyl]-2-methylpropyl](methyl)(aminocarbonyloxy)}methyl)-5-[(S)-2-[(S)-2-(14-{N-2-[4-(4-amino-1- | |
| piperidyl)-3-(6-chloro-1H-1,3-benzimidazol-2-yl)-5-(3-fluoro-5-tolyl)-2-pyridylamino]ethylcarbamoyl}-3,6,9,12- | |
| tetraoxatetradecylcarbonylamino)-3-methylbutyrylamino]-5-ureidovalerylamino]benzoic acid | |
| 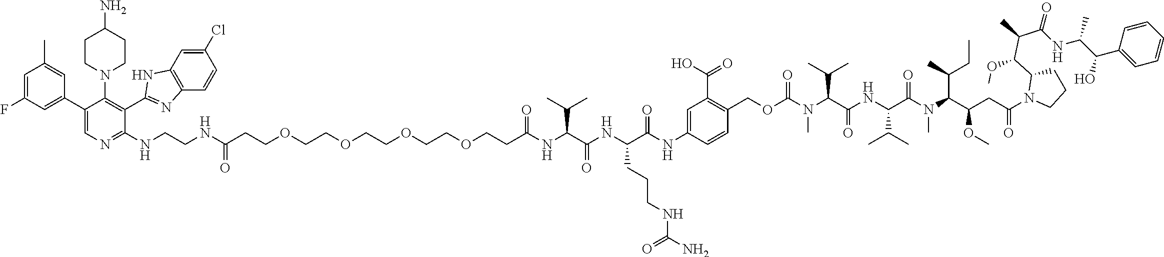 [SMILES: CC[C@H](C)[C@@H]([C@@H](CC(=O)N1CCC[C@H]1[C@H](OC)[C@@H](C)C(=O)N[C@H](C)[C@@H](O)c1ccccc1)OC)N(C)C(=O)[C@@H](NC(=O)[C@H](C(C)C)N(C)C(=O)OCc1ccc(NC(=O)[C@H](CCCNC(N)=O)NC(=O)[C@@H](NC(=O)CCOCCOCCOCCOCCC(=O)NCCNc2ncc(-c3cc(C)cc(F)c3)c(N3CCC(N)CC3)c2-c2nc3ccc(Cl)cc3[nH]2)C(C)C)cc1C(=O)O)C(C)C] | |
| (Compound 4) | |
| (S)-4-[N-(S)-1-[N-(S)-1-[N-4-({[(S)-1-{N-(S)-1-{[(1S,2R)-4-[(S)-2-{(1R,2R)-2-[N-(1R,2S)-2-hydroxy-1-methyl-2- | 1915.9 |
| phenylethylcarbamoyl]-1-methoxypropyl}-1-pyrrolidinyl]-1-[(S)-1-methylpropyl]-2-methoxy-4-oxobutyl]-N- | |
| methylcarbamoyl}-2-methylpropylcarbamoyl]-2-methylpropyl](methyl)(aminocarbonyloxy)}methyl)-3- | |
| carboxyphenylcarbamoyl]-4-ureidobutylcarbamoyl]-2-methylpropylcarbamoyl]-4-[3-(2-{N-2-[4-(4-amino-1-piperidyl)-3-(6- | |
| chloro-1H-1,3-benzimidazol-2-yl)-5-(3-fluoro-5-tolyl)-2-pyridylamino]ethylcarbamoyl}ethoxy)propionylamino]butyric acid | |
| 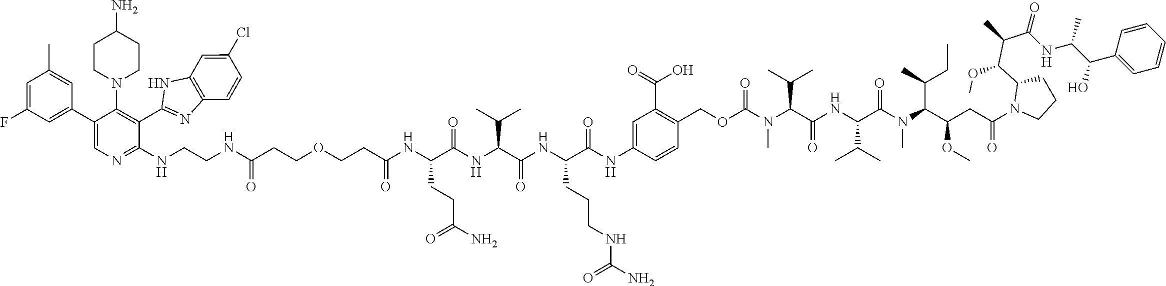 [SMILES: CC[C@H](C)[C@@H]([C@@H](CC(=O)N1CCC[C@H]1[C@H](OC)[C@@H](C)C(=O)N[C@H](C)[C@@H](O)c1ccccc1)OC)N(C)C(=O)[C@@H](NC(=O)[C@H](C(C)C)N(C)C(=O)OCc1ccc(NC(=O)[C@H](CCCNC(N)=O)NC(=O)[C@@H](NC(=O)[C@H](CCC(N)=O)NC(=O)CCOCCC(=O)NCCNc2ncc(-c3cc(C)cc(F)c3)c(N3CCC(N)CC3)c2-c2nc3ccc(Cl)cc3[nH]2)C(C)C)cc1C(=O)O)C(C)C] | |
| (Compound 5) | |
| (S)-4-[N-(S)-1-[N-(S)-1-[N-(S)-4-({[(S)-1-[N-(S)-1-{[(1S,2R)-4-[(S)-2-{(1R,2R)-2-[N-(1R,2S)-2-hydroxy-1-methyl-2- | 1939.9 |
| phenylethylcarbamoyl]-1-methoxypropyl}-1-pyrrolidinyl]-1-[(S)-1-methylpropyl]-2-methoxy-4-oxobutyl]-N- | |
| methylcarbamoyl}-2-methylpropylcarbamoyl]-2-methylpropyl](methyl)(aminocarbonyloxy)}methyl)-2- | |
| (trifluoromethyl)phenylcarbamoyl]-4-ureidobutylcarbamoyl]-2-methylpropylcarbamoyl]-4-[3-(2-{N-2-[4-(4-amino-1-piperidyl)- | |
| 3-(6-chloro-1H-1,3-benzimidazol-2-yl)-5-(3-fluoro-5-tolyl)-2-pyridylamino]ethylcarbamoyl}ethoxy)propionylamino]butyric acid | |
| 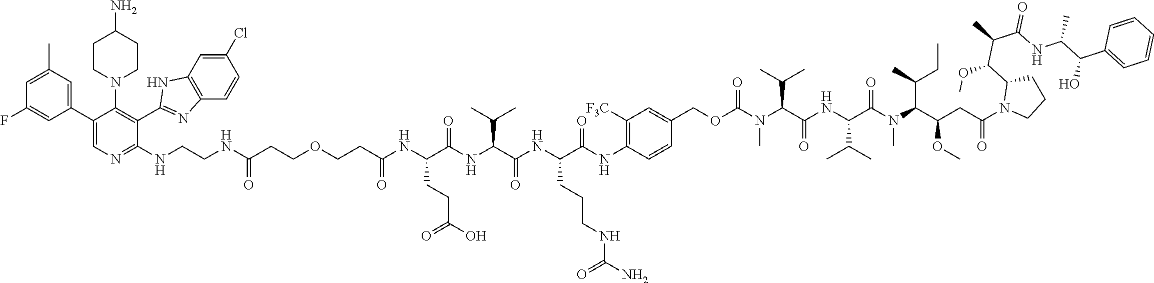 [SMILES: CC[C@H](C)[C@@H]([C@@H](CC(=O)N1CCC[C@H]1[C@H](OC)[C@@H](C)C(=O)N[C@H](C)[C@@H](O)c1ccccc1)OC)N(C)C(=O)[C@@H](NC(=O)[C@H](C(C)C)N(C)C(=O)OCc1ccc(NC(=O)[C@H](CCCNC(N)=O)NC(=O)[C@@H](NC(=O)[C@H](CCC(=O)O)NC(=O)CCOCCC(=O)NCCNc2ncc(-c3cc(C)cc(F)c3)c(N3CCC(N)CC3)c2-c2nc3ccc(Cl)cc3[nH]2)C(C)C)c(C(F)(F)F)c1)C(C)C] | |
| (Compound 6) | |
| 2-({[(S)-1-[N-(S)-1-{[(1S,2R)-4-[(S)2-{(1R,2R)-2-[N-(1R,2S)-2-hydroxy-1-methyl-2-phenylethylcarbamoyl]-1- | 1787.0 |
| methoxypropyl}-1-pyrrolidinyl]-1-[(S)-1-methylpropyl]-2-methoxy-4-oxobutyl]-N-methylcarbamoyl}-2- | |
| methylpropylcarbamoyl]-2-methylpropyl](methyl)(aminocarbonyloxy)}methyl)-5-[(S)-2-[(S)-2-[3-(2-{N-2-[4-(4-amino-1- | |
| piperidyl)-3-(6-chloro-1H-1,3-benzimidazol-2-yl)-5-(3-fluoro-5-tolyl)-2- | |
| pyridylamino]ethylcarbamoyl}ethoxy)propionylamino]-3-methylbutyrylamino]-5-ureidovalerylamino]benzoic acid | |
| 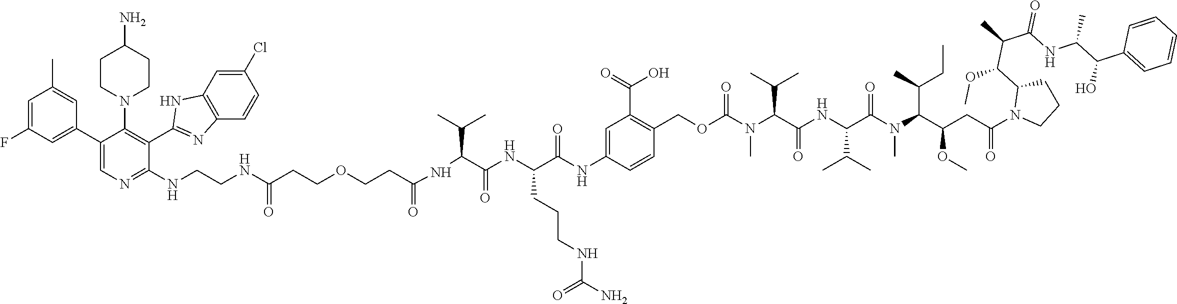 [SMILES: CC[C@H](C)[C@@H]([C@@H](CC(=O)N1CCC[C@H]1[C@H](OC)[C@@H](C)C(=O)N[C@H](C)[C@@H](O)c1ccccc1)OC)N(C)C(=O)[C@@H](NC(=O)[C@H](C(C)C)N(C)C(=O)OCc1ccc(NC(=O)[C@H](CCCNC(N)=O)NC(=O)[C@@H](NC(=O)CCOCCC(=O)NCCNc2ncc(-c3cc(C)cc(F)c3)c(N3CCC(N)CC3)c2-c2nc3ccc(Cl)cc3[nH]2)C(C)C)cc1C(=O)O)C(C)C] | |
| (Compound 7) | |
| 4-[5-({[(S)-1-[N-(S)-1-{[(1S,2R)-4-[(S)-2-{(1R,2R)-2-[N-(1R,2S)-2-hydroxy-1-methyl-2-phenylethylcarbamoyl]-1- | 1758.9 |
| methoxypropyl}-1-pyrrolidinyl]-1-[(S)-1-methylpropyl]-2-methoxy-4-oxobutyl]-N-methylcarbamoyl}-2- | |
| methylpropylcarbamoyl]-2-methylpropyl](methyl)(aminocarbonyloxy)}methyl)-2-[(S)-2-[(S)-2-[3-(2-{N-2-[4-(4-amino-1- | |
| piperidyl)-3-(6-chloro-1H-1,3-benzimidazol-2-yl)-5-(3-fluoro-5-tolyl)-2- | |
| pyridylamino]ethylcarbamoyl}ethoxy)propionylamino]-3-methylbutyrylamino]propionylamino]phenoxy]butyric acid | |
| 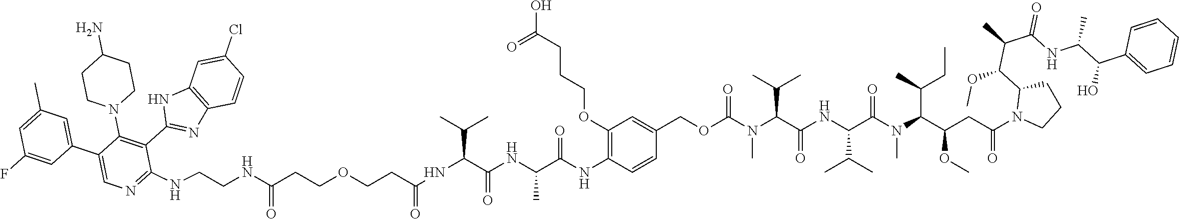 [SMILES: CC[C@H](C)[C@@H]([C@@H](CC(=O)N1CCC[C@H]1[C@H](OC)[C@@H](C)C(=O)N[C@H](C)[C@@H](O)c1ccccc1)OC)N(C)C(=O)[C@@H](NC(=O)[C@H](C(C)C)N(C)C(=O)OCc1ccc(NC(=O)[C@H](C)NC(=O)[C@@H](NC(=O)CCOCCC(=O)NCCNc2ncc(-c3cc(C)cc(F)c3)c(N3CCC(N)CC3)c2-c2nc3ccc(Cl)cc3[nH]2)C(C)C)c(OCCCC(=O)O)c1)C(C)C] | |
| (Compound 8) | |
| 1-(S)-1-[N-(S)-1-{[(1S,2R)-4-[(S)-2-{(1R,2R)-2-[N-(1R,2S)-2-hydroxy-1-methyl-2-phenylethylcarbamoyl]-1-methoxypropyl}- | 1756.9 |
| 1-pyrrolidinyl]-1-[(S)-1-methylpropyl]-2-methoxy-4-oxobutyl]-N-methylcarbamoyl}-2-methylpropylcarbamoyl]-2- | |
| methylpropyl 1-methyl {4-[(S)-2-[(S)-2-[3-(2-{N-2-[4-(4-amino-1-piperidyl)-3-(6-chloro-1H-1,3-benzimidazol-2-yl)-5-(3- | |
| fluoro-5-tolyl)-2-pyridylamino]ethylcarbamoyl}ethoxy)propionylamino]-3-methlylbutyrylamino]-5-ureidovalerylamino]-3- | |
| tolyl}methanecarbamate | |
| 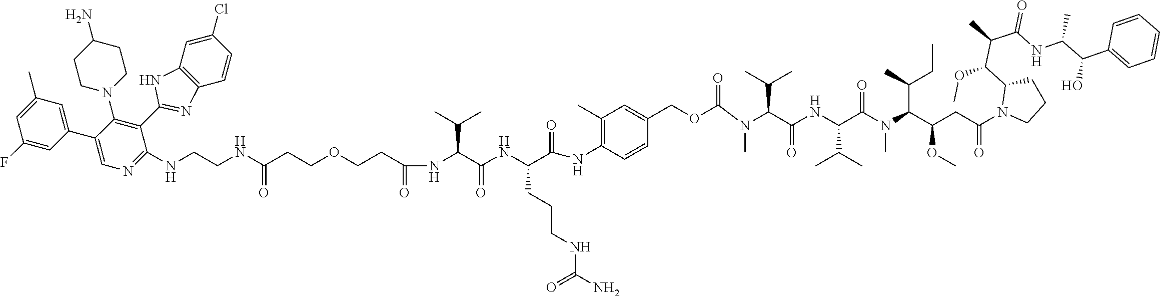 [SMILES: CC[C@H](C)[C@@H]([C@@H](CC(=O)N1CCC[C@H]1[C@H](OC)[C@@H](C)C(=O)N[C@H](C)[C@@H](O)c1ccccc1)OC)N(C)C(=O)[C@@H](NC(=O)[C@H](C(C)C)N(C)C(=O)OCc1ccc(NC(=O)[C@H](CCCNC(N)=O)NC(=O)[C@@H](NC(=O)CCOCCC(=O)NCCNc2ncc(-c3cc(C)cc(F)c3)c(N3CCC(N)CC3)c2-c2nc3ccc(Cl)cc3[nH]2)C(C)C)c(C)c1)C(C)C] | |
| (Compound 9) | |
| 1-(S)-1-[N-(S)-1-{[(1S,2R)-4-[(S)-2-{(1R,2R)-2-[N-(1R,2S)-2-hydroxy-1-methyl-2-phenylethylcarbamoyl]-1-methoxypropyl}- | 1801.9 |
| 1-pyrrolidinyl]-1-[(S)-1-methylpropyl]-2-methoxy-4-oxobutyl]-N-methylcarbamoyl}-2-methylpropylcarbamoyl]-2- | |
| methylpropyl 1-methyl {4-[(S)-2-[(S)-2-[3-(2-{N-2-[4-(4-amino-1-piperidyl)-3-(6-chloro-1H-1,3-benzimidazol-2-yl)-5-(3- | |
| fluoro-5-tolyl)-pyridylamino]ethylcarbamoyl}ethoxy)propionylamino]-3-methylbutyrylamino]-5-ureidovalerylamino]-3-(2- | |
| aminoethoxy)phenyl}methanecarbamate | |
| 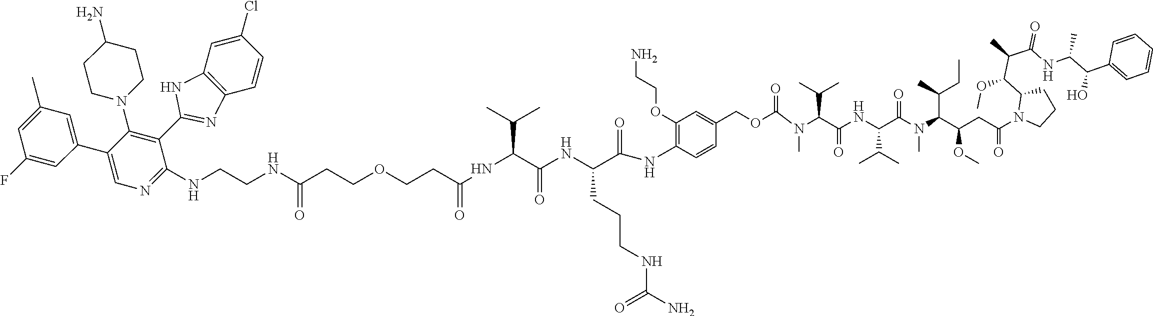 [SMILES: CC[C@H](C)[C@@H]([C@@H](CC(=O)N1CCC[C@H]1[C@H](OC)[C@@H](C)C(=O)N[C@H](C)[C@@H](O)c1ccccc1)OC)N(C)C(=O)[C@@H](NC(=O)[C@H](C(C)C)N(C)C(=O)OCc1ccc(NC(=O)[C@H](CCCNC(N)=O)NC(=O)[C@@H](NC(=O)CCOCCC(=O)NCCNc2ncc(-c3cc(C)cc(F)c3)c(N3CCC(N)CC3)c2-c2nc3ccc(Cl)cc3[nH]2)C(C)C)c(OCCN)c1)C(C)C] | |
| (Compound 10) | |
| 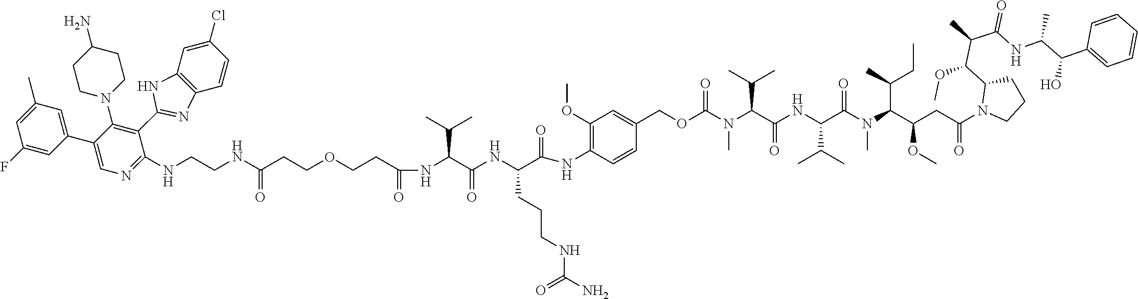 [SMILES: CC[C@H](C)[C@@H]([C@@H](CC(=O)N1CCC[C@H]1[C@H](OC)[C@@H](C)C(=O)N[C@H](C)[C@@H](O)c1ccccc1)OC)N(C)C(=O)[C@@H](NC(=O)[C@H](C(C)C)N(C)C(=O)OCc1ccc(NC(=O)[C@H](CCCNC(N)=O)NC(=O)[C@@H](NC(=O)CCOCCC(=O)NCCNc2ncc(-c3cc(C)cc(F)c3)c(N3CCC(N)CC3)c2-c2nc3ccc(Cl)cc3[nH]2)C(C)C)c(OC)c1)C(C)C] | 1773.0 |
| (Compound 11) | |
| 1-(S)-1-[N-(S)-1-{[(1S,2R)-4-[(S)-2-{(1R,2R)-2-[N-(1R,2S)-2-hydroxy-1-methyl-2-phenylethylcarbamoyl]-1-methoxypropyl}- | 1803.0 |
| 1-pyrrolidinyl]-1-[(S)-1-methylpropyl]-2-methoxy-4-oxobutyl]-N-methylcarbamoyl}-2-methylpropylcarbamoyl]-2- | |
| methylpropyl 1-methyl {4-[(S)-2-[(S)-2-[3-(2-{N-2-[4-(4-amino-1-piperidyl)-3-(6-chloro-1H-1,3-benzimidazol-2-yl)-5-(3- | |
| fluoro-5-tolyl)-2-pyridylamino]ethylcarbamoyl}ethoxy)propionylamino]-3-methylbutyrylamino]-5-ureidovalerylamino]-3- | |
| methoxyphenyl}methanecarbamate | |
| 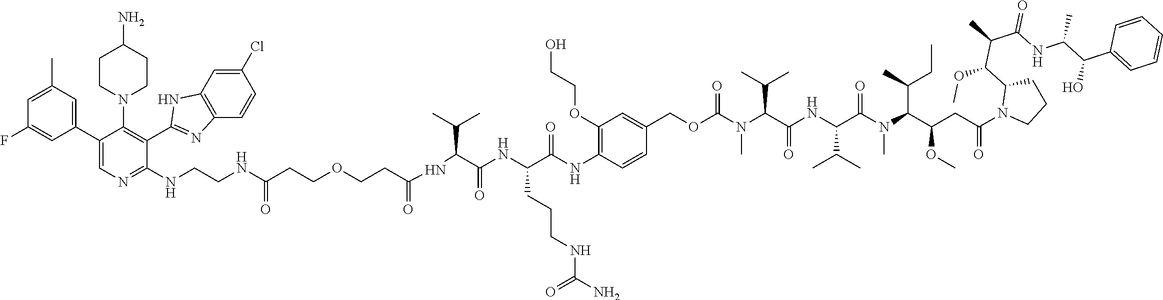 [SMILES: CC[C@H](C)[C@@H]([C@@H](CC(=O)N1CCC[C@H]1[C@H](OC)[C@@H](C)C(=O)N[C@H](C)[C@@H](O)c1ccccc1)OC)N(C)C(=O)[C@@H](NC(=O)[C@H](C(C)C)N(C)C(=O)OCc1ccc(NC(=O)[C@H](CCCNC(N)=O)NC(=O)[C@@H](NC(=O)CCOCCC(=O)NCCNc2ncc(-c3cc(C)cc(F)c3)c(N3CCC(N)CC3)c2-c2nc3ccc(Cl)cc3[nH]2)C(C)C)c(OCCO)c1)C(C)C] | |
| (Compound 12) | |
| [5-({[(S)-1-[N-(S)-1-{[(1S,2R)-4-[(S)-2-{(1R,2R)-2-[N-(1R,2S)-2-hydroxy-1-methyl-2-phenylethylcarbamoyl]-1- | 1730.9 |
| methoxypropyl}-1-pyrrolidinyl]-1-[(S)-1-methylpropyl]-2-methoxy-4-oxobutyl]-N-methylcarbamoyl}-2- | |
| methylpropylcarbamoyl]-2-methylpropyl](methyl)(aminocarbonyloxy)}methyl)-2-[(S)-2-[(S)-2-[3-(2-{N-2-[4-(4-amino-1- | |
| piperidyl)-3-(6-chloro-1H-1,3-benzimidazol-2-yl)-5-(3-fluoro-5-tolyl)-2- | |
| pyridylamino]ethylcarbamoyl}ethoxy)propionylamino]-3-methylbutyrylamino]propionylamino]phenoxy]acetic acid | |
| 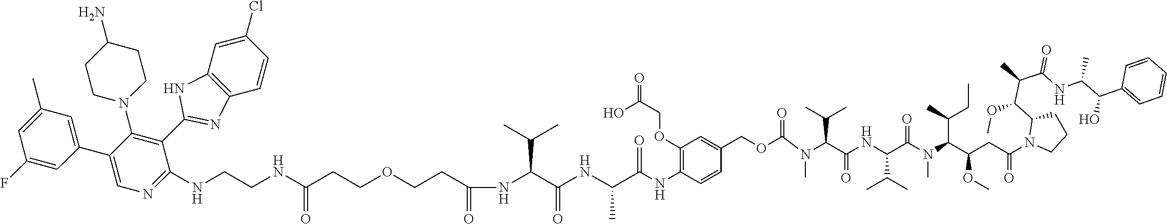 [SMILES: CC[C@H](C)[C@@H]([C@@H](CC(=O)N1CCC[C@H]1[C@H](OC)[C@@H](C)C(=O)N[C@H](C)[C@@H](O)c1ccccc1)OC)N(C)C(=O)[C@@H](NC(=O)[C@H](C(C)C)N(C)C(=O)OCc1ccc(NC(=O)[C@H](C)NC(=O)[C@@H](NC(=O)CCOCCC(=O)NCCNc2ncc(-c3cc(C)cc(F)c3)c(N3CCC(N)CC3)c2-c2nc3ccc(Cl)cc3[nH]2)C(C)C)c(OCC(=O)O)c1)C(C)C] | |
| (Compound 13) | |
| [5-({[(S)-1-[N-(S)-1-{[(1S,2R)-4-[(S)-2-{(1R,2R)-2-[N-(1R,2S)-2-hydroxy-1-methyl-2-phenylethylcarbamoyl]-1- | 1862.9 |
| methoxypropyl}-1-pyrrolidinyl]-1-[(S)-1-methylpropyl]-2-methoxy-4-oxobutyl]-N-methylcarbamoyl}-2- | |
| methylpropylcarbamoyl]-2-methylpropyl](methyl)(aminocarbonyloxy)}methyl)-2-[(S)-2-[(S)-2-(14-{N-[4-(4-amino-1- | |
| piperidyl)-3-(6-chloro-1H-1,3-benzimidazol-2-yl)-5-(3-fluoro-5-tolyl)-2-pyridylamino]ethylcarbamoyl}-3,6,9,12- | |
| tetraoxatetradecylcarbonylamino)-3-methylbutyrylamino]propionylamino]phenoxy]acetic acid | |
| 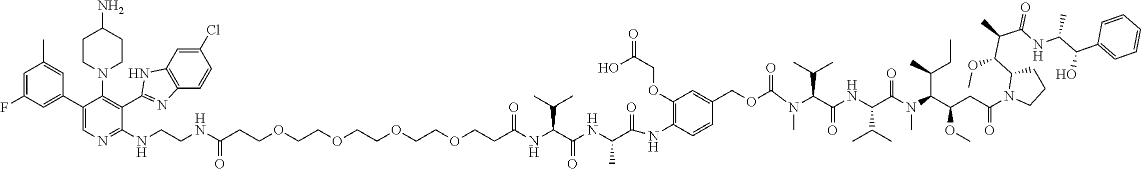 | |
| (Compound 14) | |
| (S)-4-[N-(S)-1-[N-(S)-1-[N-4-({[(S)-1-[N-(S)-1-{[(1S,2R)-4-[(S)-2-{(1R,2R)-2-[N-(1R,2S)-2-hydroxy-1-methyl-2- | 1885.7 |
| phenylethylcarbamoyl]-1-methoxypropyl}-1-pyrrolidinyl]-1-[(S)-1-methylpropyl]-2-methoxy-4-oxobutyl]-N- | |
| methylcarbamoyl}-2-methylpropylcarbamoyl]-2-methylpropyl](methyl)(aminocarbonyloxy)}methyl)-2-tolylcarbamoyl]-4- | |
| ureidobutylcarbamoyl]-2-methylpropylcarbamoyl]-4-[3-(2-{N-2-[4-(4-amino-1-piperidyl)-3-(6-chloro-1H-1,3-benzimidazol-2- | |
| yl)-5-(3-fluoro-5-tolyl)-2-pyridylamino]ethylcarbamoyl}ethoxy)propionylamino]butyric acid | |
| 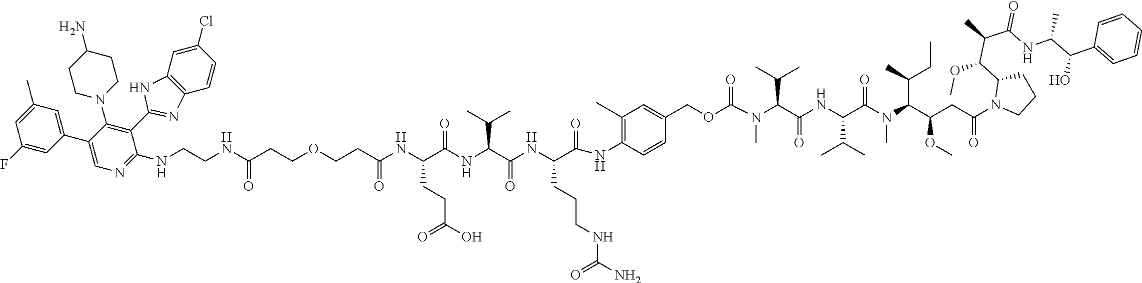 [SMILES: CC[C@H](C)[C@@H]([C@@H](CC(=O)N1CCC[C@H]1[C@H](OC)[C@@H](C)C(=O)N[C@H](C)[C@@H](O)c1ccccc1)OC)N(C)C(=O)[C@@H](NC(=O)[C@H](C(C)C)N(C)C(=O)OCc1ccc(NC(=O)[C@H](CCCNC(N)=O)NC(=O)[C@@H](NC(=O)[C@H](CCC(=O)O)NC(=O)CCOCCC(=O)NCCNc2ncc(-c3cc(C)cc(F)c3)c(N3CCC(N)CC3)c2-c2nc3ccc(Cl)cc3[nH]2)C(C)C)c(C)c1)C(C)C] | |
| (Compound 15) | |
| (S)-4-[N-(S)-1-[N-(S)-1-[N-4-({[(S)-1-[N-(S)-1-{[(1S,2R)-4-[(S)-2-{(1R,2R)-2-[N-(1R,2S)-2-hydroxy-1-methyl-2- | 1902.0 |
| phenylethylcarbamoyl]-1-methoxypropyl}-1-pyrrolidinyl]-1-[(S)-1-methylpropyl]-2-methoxy-4-oxobutyl]-N- | |
| methylcarbamoyl}-2-methylpropylcarbamoyl]-2-methylpropyl](methyl)(aminocarbonyloxy)}methyl)-2- | |
| methoxyphenylcarbamoyl]-4-ureidobutylcarbamoyl]-2-methylpropylcarbamoyl]-4-[3-(2-{N-2-[4-(4-amino-1-piperidyl)-3-(6- | |
| chloro-1H-1,3-benzimidazol-2-yl)-5-(3-fluoro-5-tolyl)-2-pyridylamino]ethylcarbamoyl}ethoxy)propionylamino]butyric acid | |
| 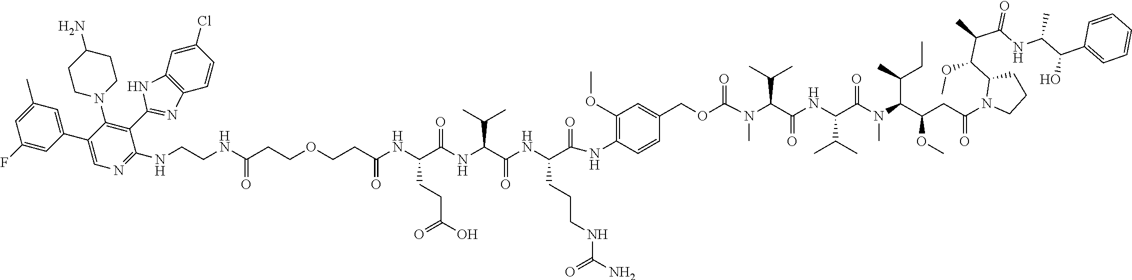 [SMILES: CC[C@H](C)[C@@H]([C@@H](CC(=O)N1CCC[C@H]1[C@H](OC)[C@@H](C)C(=O)N[C@H](C)[C@@H](O)c1ccccc1)OC)N(C)C(=O)[C@@H](NC(=O)[C@H](C(C)C)N(C)C(=O)OCc1ccc(NC(=O)[C@H](CCCNC(N)=O)NC(=O)[C@@H](NC(=O)[C@H](CCC(=O)O)NC(=O)CCOCCC(=O)NCCNc2ncc(-c3cc(C)cc(F)c3)c(N3CCC(N)CC3)c2-c2nc3ccc(Cl)cc3[nH]2)C(C)C)c(OC)c1)C(C)C] | |
| (Compound 16) | |
| 1-(S)-1-[N-(S)-1-{[(1S,2R)-4-[(S)-2-{(1R,2R)-2-[N-(1R,2S)-2-hydroxy-1-methyl-2-phenylethylcarbamoyl]-1-methoxypropyl}- | 1672.9 |
| 1-pyrrolidinyl]-1-[(S)-1-methylpropyl]-2-methoxy-4-oxobutyl]-N-methylcarbamoyl}-2-methylpropylcarbamoyl]-2- | |
| methylpropyl 1-methyl {4-[(S)-2-[(S)-2-[3-(2-{N-2-[4-(4-amino-1-piperidyl)-3-(6-chloro-1H-1,3-benzimidazol-2-yl)-5-(3- | |
| fluoro-5-tolyl)-2-pyridylamino]ethylcarbamoyl}ethoxy)propionylamino]-3-methylbutyrylamino]propionylamino]-3- | |
| hydroxyphenyl}methanecarbamate | |
| 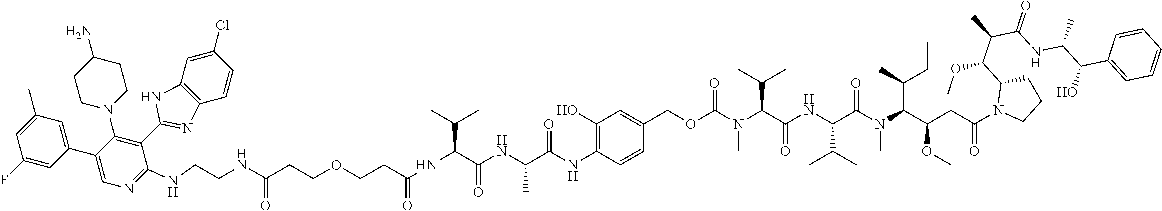 [SMILES: CC[C@H](C)[C@@H]([C@@H](CC(=O)N1CCC[C@H]1[C@H](OC)[C@@H](C)C(=O)N[C@H](C)[C@@H](O)c1ccccc1)OC)N(C)C(=O)[C@@H](NC(=O)[C@H](C(C)C)N(C)C(=O)OCc1ccc(NC(=O)[C@H](C)NC(=O)[C@@H](NC(=O)CCOCCC(=O)NCCNc2ncc(-c3cc(C)cc(F)c3)c(N3CCC(N)CC3)c2-c2nc3ccc(Cl)cc3[nH]2)C(C)C)c(O)c1)C(C)C] | |
| (Compound 17) | |
| [5-({[(S)-1-[N-(S)-1-{[(1S,2R)-4-[(S)-2-{(1R,2R)-2-[N-(1R,2S)-2-hydroxy-1-methyl-2-phenylethylcarbamoyl]-1- | 1817.0 |
| methoxypropyl}-pyrrolidinyl]-1-[(S)-1-methylpropyl]-2-methoxy-4-oxobutyl]-N-methylcarbamoyl}-2- | |
| methylpropylcarbamoyl]-2-methylpropyl](methyl)(aminocarbonyloxy)}methyl)-2-[(S)-2-[(S)-2-[3-(2-{N-2-[4-(4-amino-1- | |
| piperidyl)-3-(6-chloro-1H-1,3-benzimidazol-2-yl)-5-(3-fluoro-5-tolyl)-2- | |
| pyridylamino]ethylcarbamoyl}ethoxy)propionylamino]-3-methylbutyrylamino]-5-ureidovalerylamino]phenoxy]acetic acid | |
| 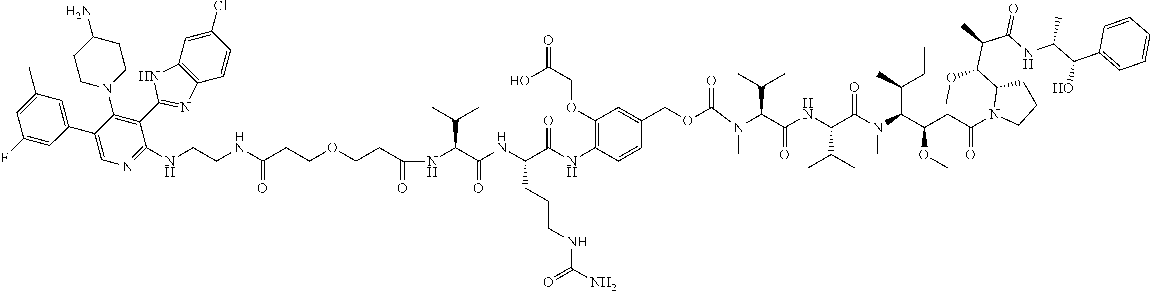 [SMILES: CC[C@H](C)[C@@H]([C@@H](CC(=O)N1CCC[C@H]1[C@H](OC)[C@@H](C)C(=O)N[C@H](C)[C@@H](O)c1ccccc1)OC)N(C)C(=O)[C@@H](NC(=O)[C@H](C(C)C)N(C)C(=O)OCc1ccc(NC(=O)[C@H](CCCNC(N)=O)NC(=O)[C@@H](NC(=O)CCOCCC(=O)NCCNc2ncc(-c3cc(C)cc(F)c3)c(N3CCC(N)CC3)c2-c2nc3ccc(Cl)cc3[nH]2)C(C)C)c(OCC(=O)O)c1)C(C)C] | |
| (Compound 18) | |
| [5-({[(S)-1-[N-(S)-1-{[(1S,2R)-4-[(S)-2-{(1R,2R)-2-[N-(1R,2S)-2-hydroxy-1-methyl-2-phenylethylcarbamoyl]-1- | 1949.0 |
| methoxypropyl}-1-pyrrolidinyl]-1-[(S)-1-methylpropyl]-2-methoxy-4-oxobutyl]-N-methylcarbamoyl}-2- | |
| methylpropylcarbamoyl]-2-methylpropyl](methyl)(aminocarbonyloxy)}methyl)-2-[(S)-2-[(S)-2-(14-{N-2-[4-(4-amino-1- | |
| piperidyl)-3-(6-chloro-1H-1,3-benzimidazol-2-yl)-5-(3-fluoro-5-tolyl)-2-pyridylamino]ethylcarbamoyl}-3,6,9,12- | |
| tetraoxatetradecylcarbonylamino)-3-methylbutyrylamino]-5-urediovalerylamino]phenoxy]acetic acid | |
| 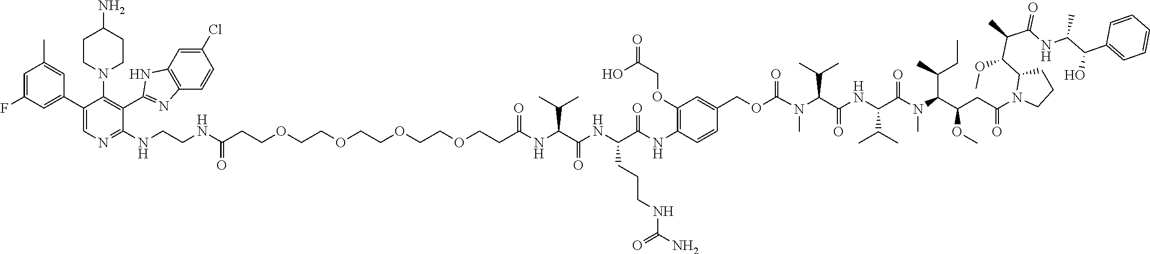 [SMILES: CC[C@H](C)[C@@H]([C@@H](CC(=O)N1CCC[C@H]1[C@H](OC)[C@@H](C)C(=O)N[C@H](C)[C@@H](O)c1ccccc1)OC)N(C)C(=O)[C@@H](NC(=O)[C@H](C(C)C)N(C)C(=O)OCc1ccc(NC(=O)[C@H](CCCNC(N)=O)NC(=O)[C@@H](NC(=O)CCOCCOCCOCCOCCC(=O)NCCNc2ncc(-c3cc(C)cc(F)c3)c(N3CCC(N)CC3)c2-c2nc3ccc(Cl)cc3[nH]2)C(C)C)c(OCC(=O)O)c1)C(C)C] | |
| (Compound 19) | |
| [5-({[(S)-1-[N-(S)-1-{[(1S,2R)-4-[(S)-2-{(1R,2R)-2-[N-(1R,2S)-2-hydroxy-1-methyl-2-phenylethylcarbamoyl]-1- | 1888.0 |
| methoxypropyl}-1-pyrrolidinyl]-1-[(S)-1-methylpropyl]-2-methoxy-4-oxobutyl]-N-methylcarbamoyl}-2- | |
| methylpropylcarbamoyl]-2-methylpropyl](methyl)(aminocarbonyloxy)}methyl)-2-[(S)-2-[(S)-2-[({N-methyl[2-(2-{N-2-[4-(4- | |
| amino-1-piperidyl)-3-(6-chloro-1H-1,3-benzimidazol-2-yl)-5-(3-fluoro-5-tolyl)-2- | |
| pyridylamino]ethylcarbamoyl}ethoxy)ethyl]carbonylamino}methyl)carbonylamino]-3-methylbutyrylamino]-5- | |
| ureidovalerylamino]phenoxy]acetic acid | |
| 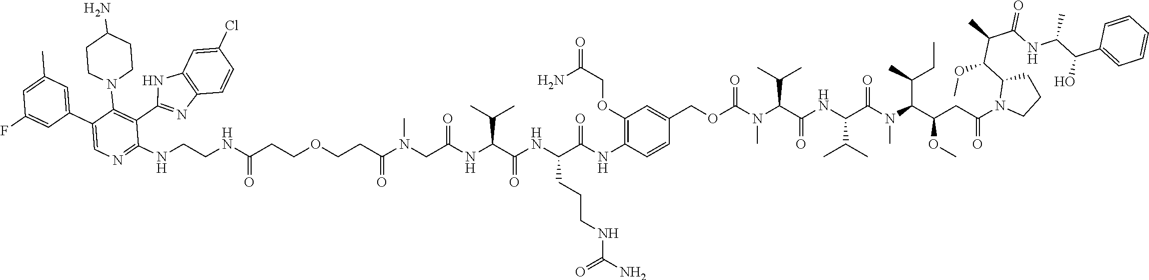 [SMILES: CC[C@H](C)[C@@H]([C@@H](CC(=O)N1CCC[C@H]1[C@H](OC)[C@@H](C)C(=O)N[C@H](C)[C@@H](O)c1ccccc1)OC)N(C)C(=O)[C@@H](NC(=O)[C@H](C(C)C)N(C)C(=O)OCc1ccc(NC(=O)[C@H](CCCNC(N)=O)NC(=O)[C@@H](NC(=O)CN(C)C(=O)CCOCCC(=O)NCCNc2ncc(-c3cc(C)cc(F)c3)c(N3CCC(N)CC3)c2-c2nc3ccc(Cl)cc3[nH]2)C(C)C)c(OCC(N)=O)c1)C(C)C] | |
| (Compound 20) | |
| [5-({[(S)-1-[N-(S)-1-{[(1S,2R)-4-[(S)-2-{(1R,2R)-2-[N-(1R,2S)-2-hydroxy-1-methyl-2-phenylethylcarbamoyl]-1- | 1888.0 |
| methoxypropyl}-1-pyrrolidinyl]-1-[(S)-1-methylpropyl]-2-methoxy-4-oxobutyl]-N-methylcarbamoyl}-2- | |
| methylpropylcarbamoyl]-2-methylpropyl](methyl)(aminocarbonyloxy)}methyl)-2-[(S)-2-[(S)-2-(3-{2-[({N-2-[4-(4-amino-1- | |
| piperidyl)-3-(6-chloro-1H-1,3-benzimidazol-2-yl)-5-(3-fluoro-5-tolyl)-2-pyridylamino]ethylcarbamoyl}methyl)-N- | |
| methylcarbamoyl]ethoxy}propionylamino)-3-methylbutyrylamino]-5-ureidovalerylamino]phenoxy}acetic acid | |
| 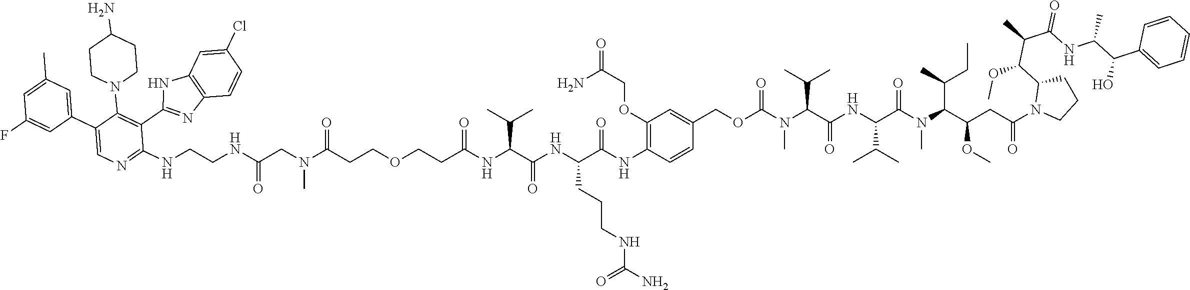 [SMILES: CC[C@H](C)[C@@H]([C@@H](CC(=O)N1CCC[C@H]1[C@H](OC)[C@@H](C)C(=O)N[C@H](C)[C@@H](O)c1ccccc1)OC)N(C)C(=O)[C@@H](NC(=O)[C@H](C(C)C)N(C)C(=O)OCc1ccc(NC(=O)[C@H](CCCNC(N)=O)NC(=O)[C@@H](NC(=O)CCOCCC(=O)N(C)CC(=O)NCCNc2ncc(-c3cc(C)cc(F)c3)c(N3CCC(N)CC3)c2-c2nc3ccc(Cl)cc3[nH]2)C(C)C)c(OCC(N)=O)c1)C(C)C] | |
| (Compound 21) | |
| [5-({[(S)-1-[N-(S)-1-{[(1S,2R)-4-[(S)-2-{(1R,2R)-2-[N-(1R,2S)-2-hydroxy-1-methyl-2-phenylethylcarbamoyl]-1- | 1903.0 |
| methoxypropyl}-1-pyrrolidinyl]-1-[(S)-1-methylpropyl]-2-methoxy-4-oxobutyl]-N-methylcarbamoyl}-2- | |
| methylpropylcarbamoyl]-2-methylpropyl](methyl)(aminocarbonyloxy)}methyl)-2-[(S)-2-[(S)-2-[(R)-3-[N-2-(2-{N-2-[4-(4- | |
| amino-1-piperidyl)-3-(6-chloro-1H-1,3-benzimidazol-2-yl)-5-(3-fluoro-5-tolyl)-2- | |
| pyridylamino]ethylcarbamoyl}ethoxy)ethylcarbamoyl]-3-aminopropionylamino]-3-methylbutyrylamino]-5- | |
| ureidovalerylamino]phenoxy]acetic acid | |
| 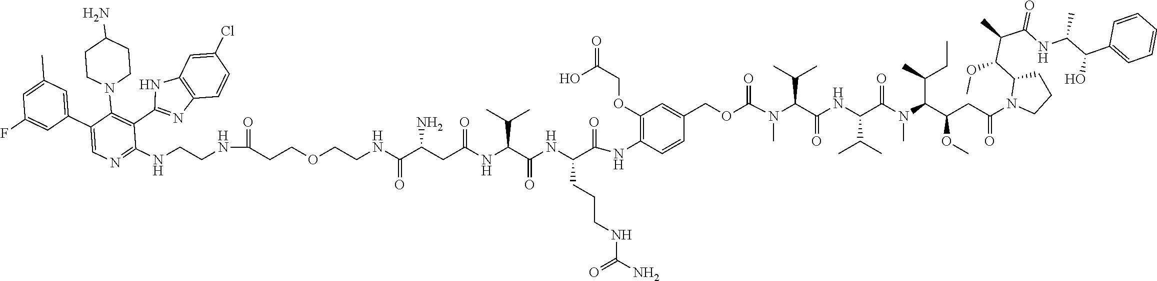 [SMILES: CC[C@H](C)[C@@H]([C@@H](CC(=O)N1CCC[C@H]1[C@H](OC)[C@@H](C)C(=O)N[C@H](C)[C@@H](O)c1ccccc1)OC)N(C)C(=O)[C@@H](NC(=O)[C@H](C(C)C)N(C)C(=O)OCc1ccc(NC(=O)[C@H](CCCNC(N)=O)NC(=O)[C@@H](NC(=O)C[C@@H](N)C(=O)NCCOCCC(=O)NCCNc2ncc(-c3cc(C)cc(F)c3)c(N3CCC(N)CC3)c2-c2nc3ccc(Cl)cc3[nH]2)C(C)C)c(OCC(=O)O)c1)C(C)C] | |
| (Compound 22) | |
| [5-({[(S)-1-[N-(S)-1-{[(1S,2R)-4-[(S)-2-{(1R,2R)-2-[N-(1R,2S)-2-hydroxy-1-methyl-2-phenylethylcarbamoyl]-1- | 2035.0 |
| methoxypropyl}-1-pyrrolidinyl]-1-[(S)-1-methylpropyl]-2-methoxy-4-oxobutyl]-N-methylcarbamoyl}-2- | |
| methylpropylcarbamoyl]-2-methylpropyl](methyl)(aminocarbonyloxy)}methyl)-2-[(S)-2-[(S)-2-[(R)-3-(N-14-{N-2-[4-(4- | |
| amino-1-piperidyl)-3-(6-chloro-1H-1,3-benzimidazol-2-yl)-5-(3-fluoro-5-tolyl)-2-pyridylamino]ethylcarbamoyl}-3,6,9,12- | |
| tetraoxatetradecylcarbamoyl)-3-aminopropionylamino]-3-methylbutyrylamino]-5-ureidovalerylamino]phenoxy]acetic acid | |
| 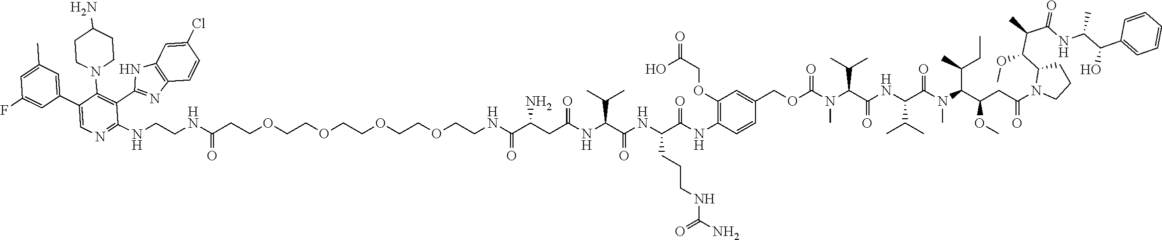 [SMILES: CC[C@H](C)[C@@H]([C@@H](CC(=O)N1CCC[C@H]1[C@H](OC)[C@@H](C)C(=O)N[C@H](C)[C@@H](O)c1ccccc1)OC)N(C)C(=O)[C@@H](NC(=O)[C@H](C(C)C)N(C)C(=O)OCc1ccc(NC(=O)[C@H](CCCNC(N)=O)NC(=O)[C@@H](NC(=O)C[C@@H](N)C(=O)NCCOCCOCCOCCOCCC(=O)NCCNc2ncc(-c3cc(C)cc(F)c3)c(N3CCC(N)CC3)c2-c2nc3ccc(Cl)cc3[nH]2)C(C)C)c(OCC(=O)O)c1)C(C)C] | |
| (Compound 23) | |
| [5-({[(S)-1-[N-(S)-1-{[(1S,2R)-4-[(S)-2-{(1R,2R)-2-[N-(1R,2S)-2-hydroxy-1-methyl-2-phenylethylcarbamoyl]-1- | 2020.0 |
| methoxypropyl}-1-pyrrolidinyl]-1-[(S)-1-methylpropyl]-2-methoxy-4-oxobutyl]-N-methylcarbamoyl}-2- | |
| methylpropylcarbamoyl]-2-methylpropyl](methyl)(aminocarbonyloxy)}methyl)-2-[(S)-2-[(S)-2-{14-[({N-2-[4-(4-amino-1- | |
| piperidyl)-3-(6-chloro-1H-1,3-benzimidazol-2-yl)-5-(3-fluoro-5-tolyl)-2-pyridylamino]ethylcarbamoyl}methyl)-N- | |
| methylcarbamoyl]-3,6,9,12-tetraoxatetradecylcarbonylamino}-3-methylbutyrylamino]-5-ureidovalerylamino]phenoxy]acetic | |
| acid | |
| 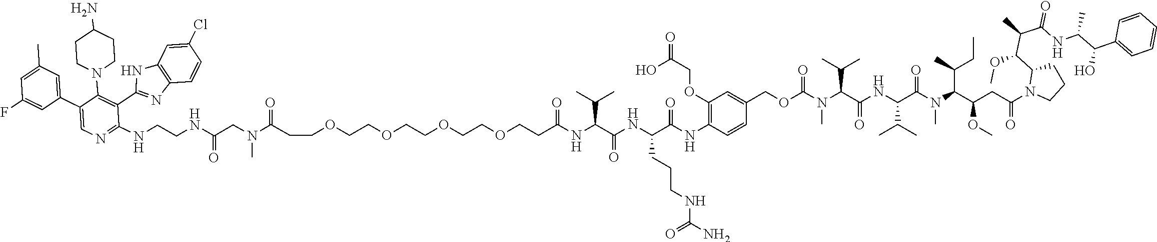 [SMILES: CC[C@H](C)[C@@H]([C@@H](CC(=O)N1CCC[C@H]1[C@H](OC)[C@@H](C)C(=O)N[C@H](C)[C@@H](O)c1ccccc1)OC)N(C)C(=O)[C@@H](NC(=O)[C@H](C(C)C)N(C)C(=O)OCc1ccc(NC(=O)[C@H](CCCNC(N)=O)NC(=O)[C@@H](NC(=O)CCOCCOCCOCCOCCC(=O)N(C)CC(=O)NCCNc2ncc(-c3cc(C)cc(F)c3)c(N3CCC(N)CC3)c2-c2nc3ccc(Cl)cc3[nH]2)C(C)C)c(OCC(=O)O)c1)C(C)C] | |
| (Compound 24) | |
| (S)-4-[N-(S)-1-[N-(S)-1-[N-4-({[(S)-1-[N-(S)-1-{[(1S,2R)-4-[(S)-2-{(1R,2R)-2-[N-(1R,2S)-2-hydroxy-1-methyl-2- | 1932.0 |
| phenylethylcarbamoyl]-1-methoxypropyl}-1-pyrrolidinyl]-1-[(S)-1-methylpropyl]-2-methoxy-4-oxobutyl]-N- | |
| methylcarbamoyl}-2-methylpropylcarbamoyl]-2-methylpropyl](methyl)(aminocarbonyloxy)}methyl)-2-(2- | |
| hydroxyethoxy)phenylcarbamoyl]-4-ureidobutylcarbamoyl]-2-methylpropylcarbamoyl]-4-[3-(2-{N-2-[4-(4-amino-1-piperidyl)- | |
| 3-(6-chloro-1H-1,3-benzimidazol-2-yl)-5-(3-fluoro-5-tolyl)-2-pyridylamino]ethylcarbamoyl}ethoxy)propionylamino]butyric | |
| acid | |
| 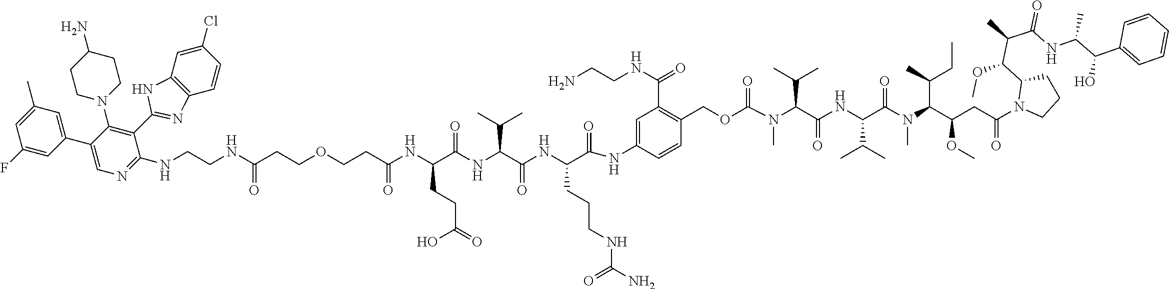 [SMILES: CC[C@H](C)[C@@H]([C@@H](CC(=O)N1CCC[C@H]1[C@H](OC)[C@@H](C)C(=O)N[C@H](C)[C@@H](O)c1ccccc1)OC)N(C)C(=O)[C@@H](NC(=O)[C@H](C(C)C)N(C)C(=O)OCc1ccc(NC(=O)[C@H](CCCNC(N)=O)NC(=O)[C@@H](NC(=O)[C@@H](CCC(=O)O)NC(=O)CCOCCC(=O)NCCNc2ncc(-c3cc(C)cc(F)c3)c(N3CCC(N)CC3)c2-c2nc3ccc(Cl)cc3[nH]2)C(C)C)cc1C(=O)NCCN)C(C)C] | |
| (Compound 25) | |
| (S)-4-[N-(S)-1-[N-(S)-1-[N-4-({[(S)-1-[N-(S)-1-{[(1S,2R)-4-[(S)-2-{(1R,2R)-2-[N-(1R,2S)-2-hydroxy-1-methyl-2- | 2063.1 |
| phenylethylcarbamoyl]-1-methoxypropyl}-1-pyrrolidinyl]-1-[(S)-1-methylpropyl]-2-methoxy-4-oxobutyl]-N- | |
| methylcarbamoyl}-2-methylpropylcarbamoyl]-2-methylpropyl](methyl)(aminocaronyloxy)}methyl)-2-(2- | |
| aminoethoxy)phenylcarbamoyl]-4-ureidobutylcarbamoyl]-2-methylpropylcarbamoyl]-4-(14-{N-2-[4-(4-amino-1-piperidyl)-3- | |
| (6-chloro-1H-1,3-benzimidazol-2-yl)-5-(3-fluoro-5-tolyl)-2-pyridylamino]ethylcarbamoyl}-3,6,9,12- | |
| tetraoxatetradecylcarbonylamino)butyric acid | |
| 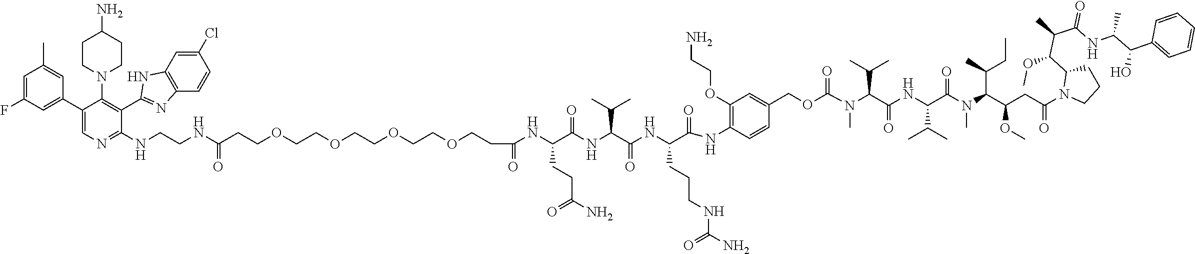 [SMILES: CC[C@H](C)[C@@H]([C@@H](CC(=O)N1CCC[C@H]1[C@H](OC)[C@@H](C)C(=O)N[C@H](C)[C@@H](O)c1ccccc1)OC)N(C)C(=O)[C@@H](NC(=O)[C@H](C(C)C)N(C)C(=O)OCc1ccc(NC(=O)[C@H](CCCNC(N)=O)NC(=O)[C@@H](NC(=O)[C@H](CCC(N)=O)NC(=O)CCOCCOCCOCCOCCC(=O)NCCNc2ncc(-c3cc(C)cc(F)c3)c(N3CCC(N)CC3)c2-c2nc3ccc(Cl)cc3[nH]2)C(C)C)c(OCCN)c1)C(C)C] | |
| (Compound 26) | |
| [5-({[(S)-1-[N-(S)-1-{[(1S,2R)-4-[(S)-2-{(1R,2R)-2-[N-(1R,2S)-2-hydroxy-1-methyl-2-phenylethylcarbamoyl]-1- | 1881.9 |
| methoxypropyl}-1-pyrrolidinyl]-1-[(S)-1-methylpropyl]-2-methoxy-4-oxobutyl]-N-methylcarbamoyl}-2- | |
| methylpropylcarbamoyl]-2-methylpropyl](methyl)(aminocarbonyloxy)}methyl)-2-[(S)-2-[(S)-2-[3-(2-{N-2-[4-(4-amino- | |
| 1-piperidyl)-3-(6-chloro-1H-1,3-benzimidazol-2-yl)-5-(3-fluoro-5-tolyl)-2- | |
| pyridylamino]ethylcarbamoyl}ethoxy)propionylamino]-3-sulfopropionylamino]-3- | |
| methylbutyrylamino]propionylamino]phenoxy]acetic acid | |
| 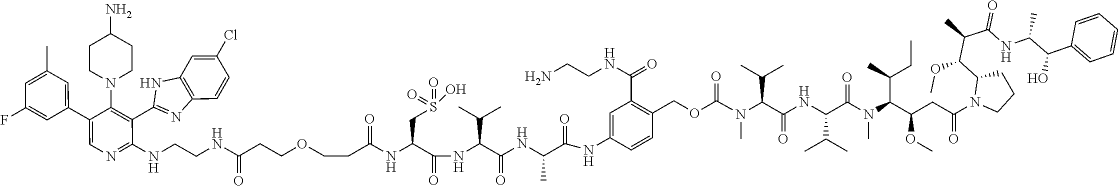 [SMILES: CC[C@H](C)[C@@H]([C@@H](CC(=O)N1CCC[C@H]1[C@H](OC)[C@@H](C)C(=O)N[C@H](C)[C@@H](O)c1ccccc1)OC)N(C)C(=O)[C@@H](NC(=O)[C@H](C(C)C)N(C)C(=O)OCc1ccc(NC(=O)[C@H](C)NC(=O)[C@@H](NC(=O)[C@H](CS(=O)(=O)O)NC(=O)CCOCCC(=O)NCCNc2ncc(-c3cc(C)cc(F)c3)c(N3CCC(N)CC3)c2-c2nc3ccc(Cl)cc3[nH]2)C(C)C)cc1C(=O)NCCN)C(C)C] | |
| (Compound 27) | |
| [5-({[(S)-1-[N-(S)-1-{[(1S,2R)-4-[(S)-2-{(1R,2R)-2-[N-(1R,2S)-2-hydroxy-1-methyl-2-phenylethylcarbamoyl]-1- | 1967.9 |
| methoxypropyl}-pyrrolidinyl]-1-[(S)-1-methylpropyl]-2-methoxy-4-oxobutyl]-N-methylcarbamoyl}-2- | |
| methylpropylcarbamoyl]-2-methylpropyl](methyl)(aminocarbonyloxy)}methyl)-2-[(S)-2-[(S)-2-[3-(2-{N-2-[4-(4-amino- | |
| 1-piperidyl)-3-(6-chloro-1H-1,3-benzimidazol-2-yl)-5-(3-fluoro-5-tolyl)-2- | |
| pyridylamino]ethylcarbamoyl}ethoxy)propionylamino]-3-sulfopropionylamino]-3-methylbutyrylamino]-5- | |
| ureidovalerylamino]phenoxy]acetic acid | |
| 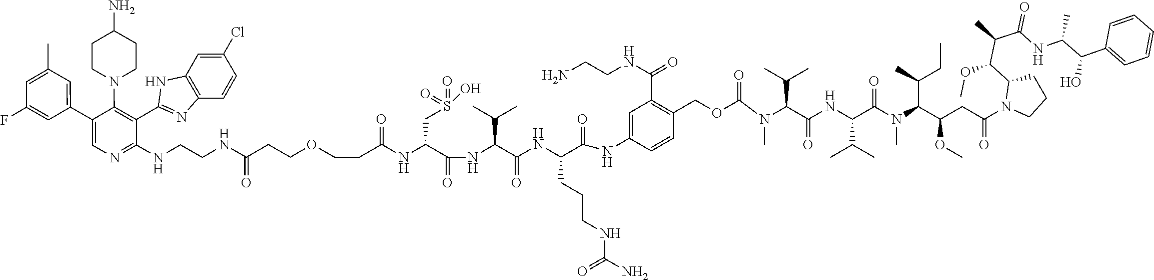 [SMILES: CC[C@H](C)[C@@H]([C@@H](CC(=O)N1CCC[C@H]1[C@H](OC)[C@@H](C)C(=O)N[C@H](C)[C@@H](O)c1ccccc1)OC)N(C)C(=O)[C@@H](NC(=O)[C@H](C(C)C)N(C)C(=O)OCc1ccc(NC(=O)[C@H](CCCNC(N)=O)NC(=O)[C@@H](NC(=O)[C@@H](CS(=O)(=O)O)NC(=O)CCOCCC(=O)NCCNc2ncc(-c3cc(C)cc(F)c3)c(N3CCC(N)CC3)c2-c2nc3ccc(Cl)cc3[nH]2)C(C)C)cc1C(=O)NCCN)C(C)C] | |
| (Compound 28) | |
| (S)-2-[N-(S)-1-[N-(S)-1-[N-4-({[(S)-1-[N-(S)-1-{[(1S,2R)-4-[(S)-2-{(1R,2R)-2-[N-(1R,2S)-2-hydroxy-1-methyl-2- | 1952.9 |
| phenylethylcarbamoyl]-1-methoxypropyl}-1-pyrrolidinyl]-1-[(S)-1-methylpropyl]-2-methoxy-4-oxobutyl]-N- | |
| methylcarbamoyl}-2-methylpropylcarbamoyl]-2-methylpropyl](methyl)(aminocarbonyloxy)}methyl)-2-(2- | |
| aminoethoxy)phenylcarbamoyl]-4-ureidobutylcarbamoyl]-2-methylpropylcarbamoyl]-2-[3-(2-{N-2-[4-(4-amino-1-piperidyl)-3- | |
| (6-chloro-1H-1,3-benzimidazol-2-yl)-5-(3-fluoro-5-tolyl)-2- | |
| pyridylamino]ethylcarbamoyl}ethoxy)propionylamino]ethanesulfonic acid | |
| 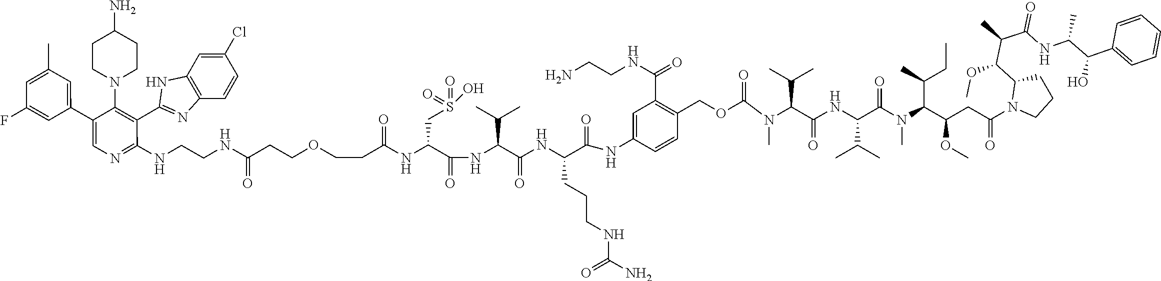 [SMILES: CC[C@H](C)[C@@H]([C@@H](CC(=O)N1CCC[C@H]1[C@H](OC)[C@@H](C)C(=O)N[C@H](C)[C@@H](O)c1ccccc1)OC)N(C)C(=O)[C@@H](NC(=O)[C@H](C(C)C)N(C)C(=O)OCc1ccc(NC(=O)[C@H](CCCNC(N)=O)NC(=O)[C@@H](NC(=O)[C@@H](CS(=O)(=O)O)NC(=O)CCOCCC(=O)NCCNc2ncc(-c3cc(C)cc(F)c3)c(N3CCC(N)CC3)c2-c2nc3ccc(Cl)cc3[nH]2)C(C)C)cc1C(=O)NCCN)C(C)C] | |
| (Compound 29) | |
| {2-[(S)-2-[(S)-2-[(S)-3-hydroxy-2-[3-(2-{N-2-[4-(4-amino-1-piperidyl)-3-(6-chloro-1H-1,3-benzimidazol-2-yl)-5-(3-fluoro-5- | 1903.9 |
| tolyl)-2-pyridylamino]ethylcarbamoyl}ethoxy)propionylamino]propionylamino]-3-methylbutyrylamino]-5-ureidovalerylamino]- | |
| 5-({methyl[(S)-2-methyl-1-[N-(S)-1-{[(1S,2R)-2-methoxy-4-[(S)-2-[(1R,2R)-1-methoxy-2-[N-(1R,2S)-2-hydroxy-1-methyl-2- | |
| phenylethylcarbamoyl]propyl]-1-pyrrolidinyl]-1-[(S)-1-methylpropyl]-4-oxobutyl]-N-methylcarbamoyl}-2- | |
| methylpropylcarbamoyl]propyl](aminocarbonyloxy)}methyl)phenoxy}acetic acid | |
| 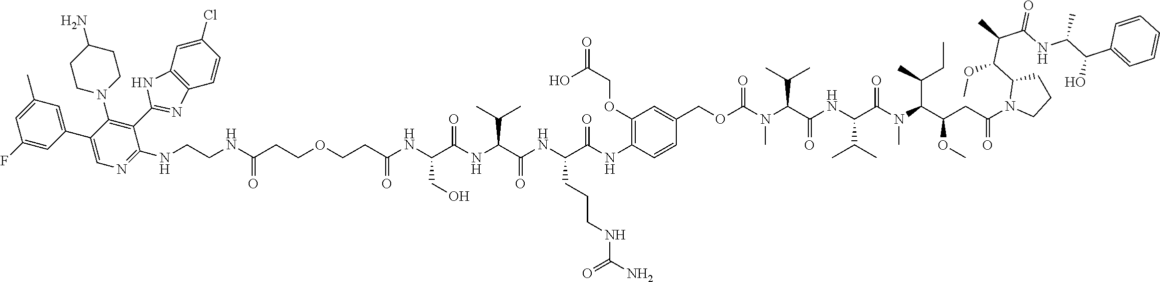 [SMILES: CC[C@H](C)[C@@H]([C@@H](CC(=O)N1CCC[C@H]1[C@H](OC)[C@@H](C)C(=O)N[C@H](C)[C@@H](O)c1ccccc1)OC)N(C)C(=O)[C@@H](NC(=O)[C@H](C(C)C)N(C)C(=O)OCc1ccc(NC(=O)[C@H](CCCNC(N)=O)NC(=O)[C@@H](NC(=O)[C@H](CO)NC(=O)CCOCCC(=O)NCCNc2ncc(-c3cc(C)cc(F)c3)c(N3CCC(N)CC3)c2-c2nc3ccc(Cl)cc3[nH]2)C(C)C)c(OCC(=O)O)c1)C(C)C] | |
| (Compound 30) | |
| 1-methyl 1-(S)-2-methyl-1-[N-(S)-1-{[(1S,2R)-2-methoxy-4-[(S)-2-[(1R,2R)-1-methoxy-2-[N-(1R,2S)-2-hydroxy-1-methyl-2- | 1843.9 |
| phenylethylcarbamoyl]propyl]-1-pyrrolidinyl]-1-[(S)-1-methylpropyl]-4-oxobutyl]-N-methylcarbamoyl}-2- | |
| methylpropylcarbamoyl]propyl {3-methoxy-4-[(S)-2-[(S)-3-methyl-2-(3-{2-[N-methyl({N-2-[4-(4-amino-1-piperidyl)-3-(6- | |
| chloro-1H-1,3-benzimidazol-2-yl)-5-(3-fluoro-5-tolyl)-2- | |
| pyridylamino]ethylcarbamoyl}methyl)carbamoyl]ethoxy}propionylamino)butyrylamino]-5- | |
| ureidovalerylamino]phenyl}methanecarbamate | |
| 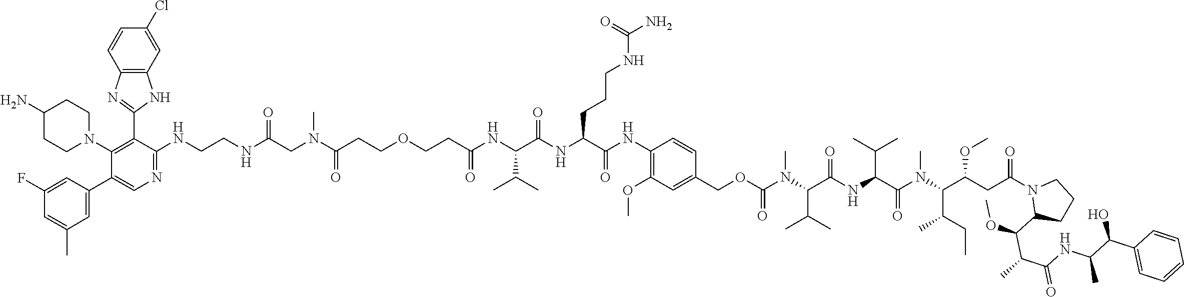 | |
| (Compound 198) | |
| 1-methyl 1-(S)-2-methyl-1-[N-(S)-1-{[(1S,2R)-2-methoxy-4-[(S)-2-[(1R,2R)-1-methoxy-2-[N-(1R,2S)-2-hydroxy-1-methyl-2- | 1829.9 |
| phenylethylcarbamoyl]propyl]-1-pyrrolidinyl]-1-[(S)-1-methylpropyl]-4-oxobutyl]-N-methylcarbamoyl}-2- | |
| methylpropylcarbamoyl]propyl {3-hydroxy-4-[(S)-2-[(S)-3-methyl-2-(3-{2-[N-methyl({N-2-[4-(4-amino-1-piperidyl)-3-(6- | |
| chloro-1H-1,3-benzimidazol-2-yl)-5-(3-fluoro-5-tolyl)-2- | |
| pyridylamino]ethylcarbamoyl}methyl)carbamoyl]ethoxy}propionylamino)butyrylamino]-5- | |
| ureidovalerylamino]phenyl}methanecarbamate | |
| 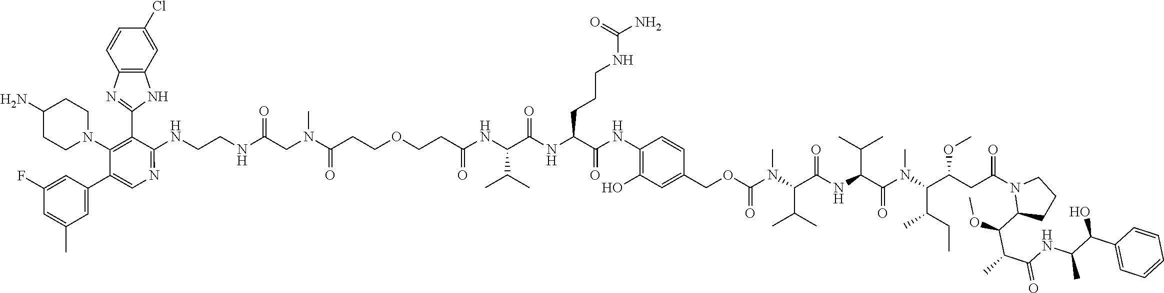 [SMILES: CC[C@H](C)[C@@H]([C@@H](CC(=O)N1CCC[C@H]1[C@H](OC)[C@@H](C)C(=O)N[C@H](C)[C@@H](O)c1ccccc1)OC)N(C)C(=O)[C@@H](NC(=O)[C@H](C(C)C)N(C)C(=O)OCc1ccc(NC(=O)[C@H](CCCNC(N)=O)NC(=O)[C@@H](NC(=O)CCOCCC(=O)N(C)CC(=O)NCCNc2ncc(-c3cc(C)cc(F)c3)c(N3CCC(N)CC3)c2-c2nc3ccc(Cl)cc3[nH]2)C(C)C)c(O)c1)C(C)C] | |
| (Compound 199) | |
| (S)-2-{2-[(S)-2-[(S)-2-[(R)-3-amino-3-[N-2-(2-{N-[4-(4-amino-1-piperidyl)-3-(6-chloro-1H-1,3-benzimidazol-2-yl)-5-(3- | 1917.0 |
| fluoro-5-tolyl)-2-pyridylamino]ethylcarbamoyl}ethoxy)ethylcarbamoyl]propionylamino]-3-methylbutyrylamino]-5- | |
| ureidovalerylamino]-5-({methyl[(S)-2-methyl-1-[N-(S)-1-{[(1S,2R)-2-methoxy-4-[(S)-2-[(1R,2R)-1-methoxy-2-[N-(1R,2S)-2- | |
| hydroxy-1-methyl-2-phenylethylcarbamoyl]propyl]-1-pyrrolidinyl]-1-[(S)-1-methylpropyl]-4-oxobutyl]-N-methylcarbamoyl}-2- | |
| methylpropylcarbamoyl]propyl](aminocarbonyloxy)}methyl)phenoxy}propionic acid | |
| 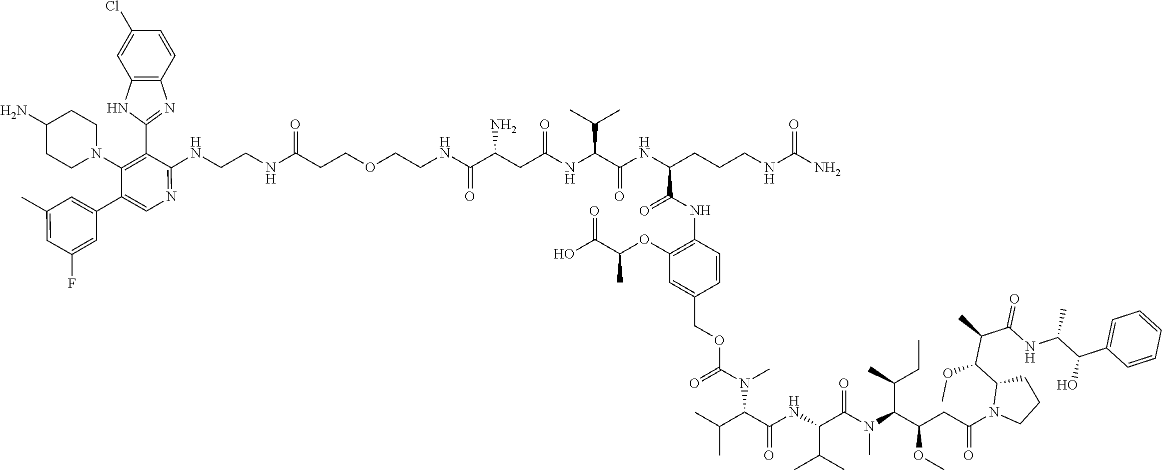 [SMILES: CC[C@H](C)[C@@H]([C@@H](CC(=O)N1CCC[C@H]1[C@H](OC)[C@@H](C)C(=O)N[C@H](C)[C@@H](O)c1ccccc1)OC)N(C)C(=O)[C@@H](NC(=O)[C@H](C(C)C)N(C)C(=O)OCc1ccc(NC(=O)[C@H](CCCNC(N)=O)NC(=O)[C@@H](NC(=O)C[C@@H](N)C(=O)NCCOCCC(=O)NCCNc2ncc(-c3cc(C)cc(F)c3)c(N3CCC(N)CC3)c2-c2nc3ccc(Cl)cc3[nH]2)C(C)C)c(O[C@@H](C)C(=O)O)c1)C(C)C] | |
| (Compound 200) | |
| (R)-2-{2-[(S)-2-[(S)-2-[(R)-3-amino-3-[N-2-(2-{N-2-[4-(4-amino-1-piperidyl)-3-(6-chloro-1H-1,3-benzimidazol-2-yl)-5-(3- | 1917.0 |
| fluoro-5-tolyl)-2-pyridylamino]ethylcarbamoyl}ethoxy)ethylcarbamoyl]propionylamino]-3-methylbutyrylamino]-5- | |
| ureidovalerylamino]-5-({methyl[(S)-2-methyl-1-[N-(S)-1-{[(1S,2R)-2-methoxy-4-[(S)-2-[(1R,2R)-1-methoxy-2-[N-(1R,2S)-2- | |
| hydroxy-1-methyl-2-phenylethylcarbamoyl]propyl]-1-pyrrolidinyl]-1-[(S)-1-methylpropyl]-4-oxobutyl]-N-methylcarbamoyl}-2- | |
| methylpropylcarbamoyl]propyl](aminocarbonyloxy)}methyl)phenoxy}propionic acid | |
| 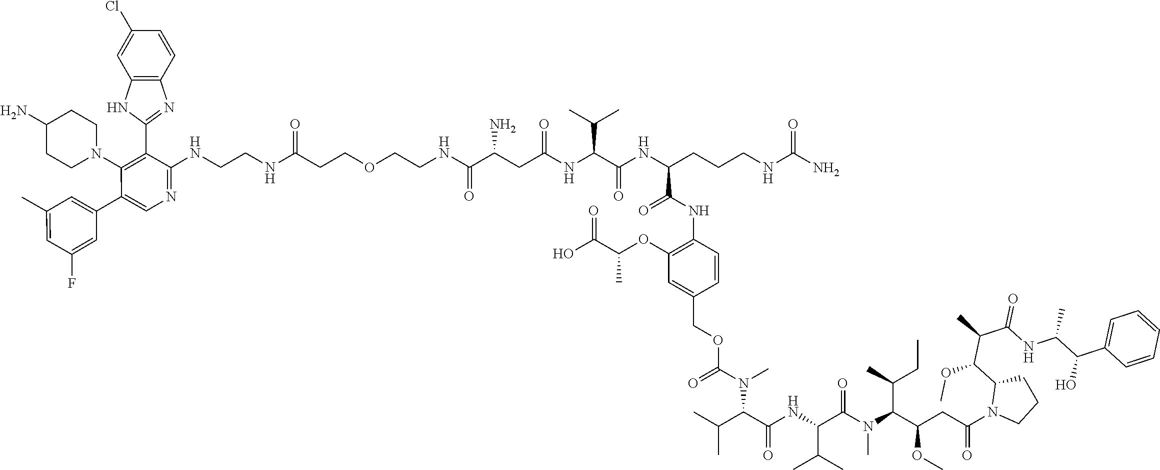 [SMILES: CC[C@H](C)[C@@H]([C@@H](CC(=O)N1CCC[C@H]1[C@H](OC)[C@@H](C)C(=O)N[C@H](C)[C@@H](O)c1ccccc1)OC)N(C)C(=O)[C@@H](NC(=O)[C@H](C(C)C)N(C)C(=O)OCc1ccc(NC(=O)[C@H](CCCNC(N)=O)NC(=O)[C@@H](NC(=O)C[C@@H](N)C(=O)NCCOCCC(=O)NCCNc2ncc(-c3cc(C)cc(F)c3)c(N3CCC(N)CC3)c2-c2nc3ccc(Cl)cc3[nH]2)C(C)C)c(O[C@H](C)C(=O)O)c1)C(C)C] | |
| (Compound 201) | |
| {2-[(S)-2-[(S)-2-[(S)-3-(4-imidazolyl)-2-[3-(2-{N-2-[4-(4-amino-1-piperidyl)-3-(6-chloro-1H-1,3-benzimidazol-2-yl)-5-(3- | 1953.9 |
| fluoro-5-tolyl)-2-pyridylamino]ethylcarbamoyl}ethoxy)propionylamino]propionylamino]-3-methylbutyrylamino]-5- | |
| ureidovalerylamino]-5-({methyl[(S)-2-methyl-1-[N-(S)-1-{[(1S,2R)-2-methoxy-4-[(S)-2-[(1R,2R)-1-methoxy-2-[N-(1R,2S)-2- | |
| hydroxy-1-methyl-2-phenylethylcarbamoyl]propyl]-1-pyrrolidinyl]-1-[(S)-1-methylpropyl]-4-oxobutyl]-N-methylcarbamoyl}-2- | |
| methylpropylcarbamoyl]propyl](aminocarbonyloxy)}methyl)phenoxy}acetic acid | |
| 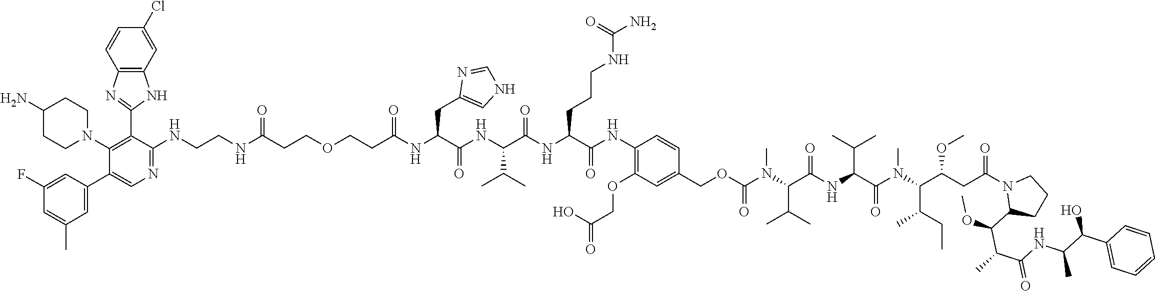 | |
| (Compound 202) | |
| [5-({methyl[(S)-2-methyl-1-[N-(S)-1-{[(1S,2R)-2-methoxy-4-[(S)-2-[(1R,2R)-1-methoxy-2-[N-(1R,2S)-2-hydroxy-1-methyl-2- | 1953.9 |
| phenylethylcarbamoyl]propyl]-1-pyrrolidinyl]-1-[(S)-1-methylpropyl]-4-oxobutyl]-N-methylcarbamoyl}-2- | |
| methylpropylcarbamoyl]propyl](aminocarbonyloxy)}methyl)-2-[(S)-2-[(S)-3-methyl-2-{[(S)-(1-methyl-4-pyrazolyl)[3-(2-{N-2- | |
| [4-(4-amino-1-piperidyl)-3-(6-chloro-1H-1,3-benzimidazol-2-yl)-5-(3-fluoro-5-tolyl)-2- | |
| pyridylamino]ethylcarbamoyl}ethoxy)propionylamino]methyl]carbonylamino}butyrylamino]-5- | |
| ureidovalerylamino]phenoxy]acetic acid | |
| 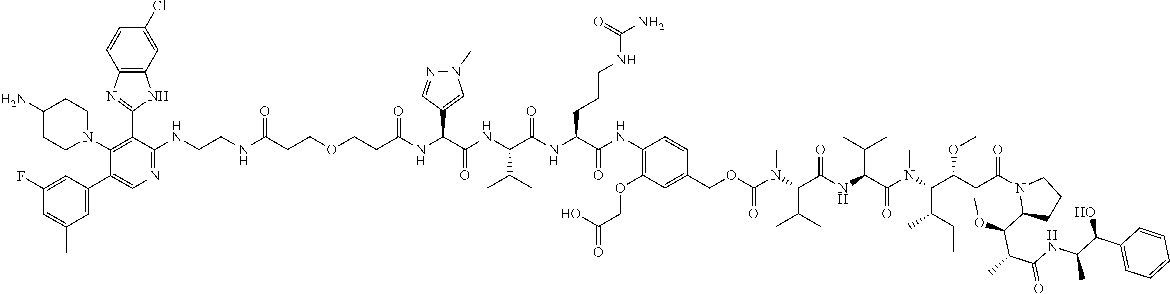 [SMILES: CC[C@H](C)[C@@H]([C@@H](CC(=O)N1CCC[C@H]1[C@H](OC)[C@@H](C)C(=O)N[C@H](C)[C@@H](O)c1ccccc1)OC)N(C)C(=O)[C@@H](NC(=O)[C@H](C(C)C)N(C)C(=O)OCc1ccc(NC(=O)[C@H](CCCNC(N)=O)NC(=O)[C@@H](NC(=O)[C@@H](NC(=O)CCOCCC(=O)NCCNc2ncc(-c3cc(C)cc(F)c3)c(N3CCC(N)CC3)c2-c2nc3ccc(Cl)cc3[nH]2)c2cnn(C)c2)C(C)C)c(OCC(=O)O)c1)C(C)C] | |
| (Compound 203) | |
| [5-({methyl[(S)-2-methyl-1-[N-(S)-1-{[(1S,2R)-2-methoxy-4-[(S)-2-[(1R,2R)-1-methoxy-2-[N-(1R,2S)-2-hydroxy-1-methyl-2- | 1953.9 |
| phenylethylcarbamoyl]propyl]-1-pyrrolidinyl]-1-[(S)-1-methylpropyl]-4-oxobutyl]-N-methylcarbamoyl}-2- | |
| methylpropylcarbamoyl]propyl](aminocarbonyloxy)}methyl)-2-[(S)-2-[(S)-3-methyl-2-{[(R)-(1-methyl-4-pyrazolyl)[3-(2-{N-2- | |
| [4-(4-amino-1-piperidyl)-3-(6-chloro-1H-1,3-benzimidazol-2-yl)-5-(3-fluoro-5-tolyl)-2- | |
| pyridylamino]ethylcarbamoyl}ethoxy)propionylamino]methyl]carbonylamino}butyrylamino]-5- | |
| ureidovalerylamino]phenoxy]acetic acid | |
| 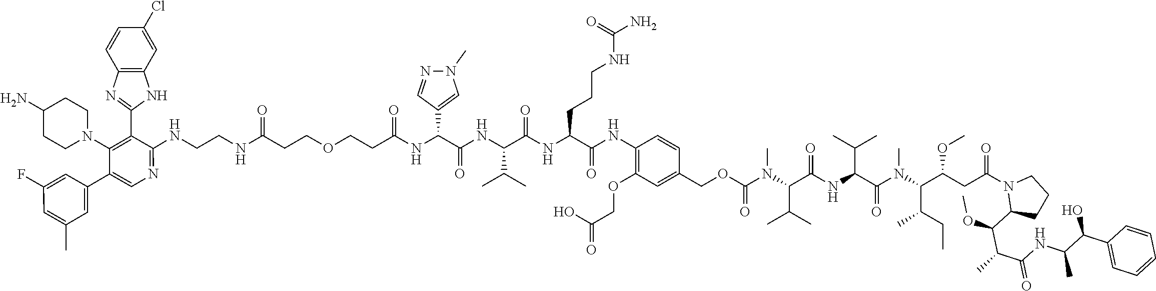 [SMILES: CC[C@H](C)[C@@H]([C@@H](CC(=O)N1CCC[C@H]1[C@H](OC)[C@@H](C)C(=O)N[C@H](C)[C@@H](O)c1ccccc1)OC)N(C)C(=O)[C@@H](NC(=O)[C@H](C(C)C)N(C)C(=O)OCc1ccc(NC(=O)[C@H](CCCNC(N)=O)NC(=O)[C@@H](NC(=O)[C@H](NC(=O)CCOCCC(=O)NCCNc2ncc(-c3cc(C)cc(F)c3)c(N3CCC(N)CC3)c2-c2nc3ccc(Cl)cc3[nH]2)c2cnn(C)c2)C(C)C)c(OCC(=O)O)c1)C(C)C] | |
| (Compound 204) | |
| 3-{2-[(S)-2-[(S)-2-[(R)-3-amino-3-[N-2-(2-{N-2-[4-(4-amino-1-piperidyl)-3-(6-chloro-1H-1,3-benzimidazol-2-yl)-5-(3-fluoro- | 1901.0 |
| 5-tolyl)-2-pyridylamino]ethylcarbamoyl}ethoxy)ethylcarbamoyl]propionylamino]-3-methylbutyrylamino]-5- | |
| ureidovalerylamino]-5-({methyl[(S)-2-methyl-1-[N-(S)-1-{[(1S,2R)-2-methoxy-4-[(S)-2-[(1R,2R)-1-methoxy-2-[N-(1R,2S)-2- | |
| hydroxy-1-methyl-2-phenylethylcarbamoyl]propyl]-1-pyrrolidinyl]-1-[(S)-1-methylpropyl]-4-oxobutyl]-N-methylcarbamoyl}-2- | |
| methylpropylcarbamoyl]propyl](aminocarbonyloxy)}methyl)phenyl}propionic acid | |
| 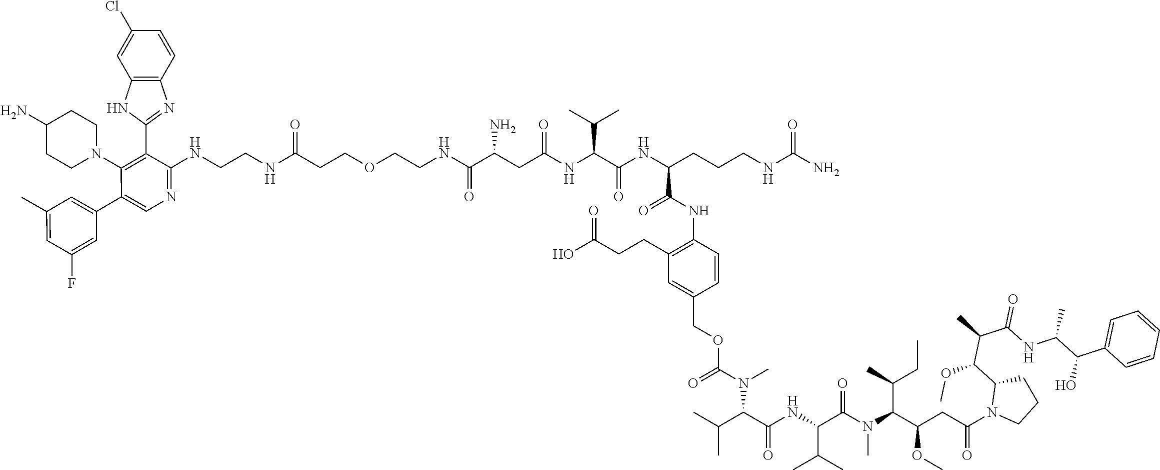 [SMILES: CC[C@H](C)[C@@H]([C@@H](CC(=O)N1CCC[C@H]1[C@H](OC)[C@@H](C)C(=O)N[C@H](C)[C@@H](O)c1ccccc1)OC)N(C)C(=O)[C@@H](NC(=O)[C@H](C(C)C)N(C)C(=O)OCc1ccc(NC(=O)[C@H](CCCNC(N)=O)NC(=O)[C@@H](NC(=O)C[C@@H](N)C(=O)NCCOCCC(=O)NCCNc2ncc(-c3cc(C)cc(F)c3)c(N3CCC(N)CC3)c2-c2nc3ccc(Cl)cc3[nH]2)C(C)C)c(CCC(=O)O)c1)C(C)C] | |
| (Compound 205) | |

### Example B. 1-(S)-1-[N—(S)-1-{[(1S,2R)-4-[(S)-2-{(1R,2R)-2-[N-(1R,2S)-2-hydroxy-1-methyl-2-phenylethylcarbamoyl]-1-methoxypropy})-1-pyrrolidinyl]-1-[(S)-1-methylpropyl]-2-methoxy-4-oxobutyl]-N-methylcarbamoyl}-2-methylpropylcarbamoyl]-2-methylpropyl 1-methyl {3-[(2S,3R,4S,5S,6S)-6-(N-2-aminoethylcarbamoyl)-3,4,5-trihydroxytetrahydro-2H-pyran-2-yloxy]-4-[(S)-2-[(S)-2-(3-{2-[({N-2-[4-(4-amino-1-piperidyl)-3-(6-chloro-1H-1,3-benzimidazol-2-yl)-5-(3-fluoro-5-tolyl)-2-pyridylamino]ethylcarbamoyl}methyl)-N-methylcarbamoyl]ethoxy}propionylamino)-3-methylbutyrylamino]propionylamino]phenyl}methanecarbamate (Compound 31)

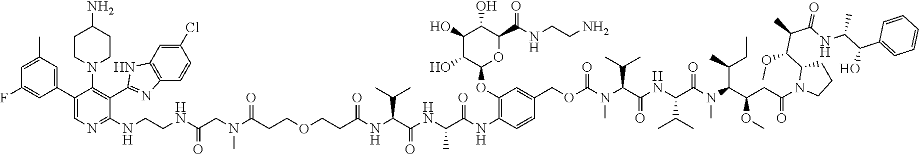 [SMILES: CC[C@H](C)[C@@H]([C@@H](CC(=O)N1CCC[C@H]1[C@H](OC)[C@@H](C)C(=O)N[C@H](C)[C@@H](O)c1ccccc1)OC)N(C)C(=O)[C@@H](NC(=O)[C@H](C(C)C)N(C)C(=O)OCc1ccc(NC(=O)[C@H](C)NC(=O)[C@@H](NC(=O)CCOCCC(=O)N(C)CC(=O)NCCNc2ncc(-c3cc(C)cc(F)c3)c(N3CCC(N)CC3)c2-c2nc3ccc(Cl)cc3[nH]2)C(C)C)c(O[C@@H]2O[C@H](C(=O)NCCN)[C@@H](O)[C@H](O)[C@H]2O)c1)C(C)C]

Step B-1, preparation of (9H-fluoren-9-yl)methyl (2-((2-((4-(4-((tert-butoxycarbonyl)amino)piperidin-1-yl)-3-(6-chloro-1H-benzo[d]imidazol-2-yl)-5-(3-fluoro-5-methylphenyl)pyridin-2-yl)amino)ethyl)amino)-2-oxoethyl)(methyl)carbamate: Into a 8-mL vial was placed was mixture of tert-butyl (1-(2-((2-aminoethyl)amino)-3-(6-chloro-1H-benzo[d]imidazol-2-yl)-5-(3-fluoro-5-methylphenyl)pyridin-4-yl)piperidin-4-yl)carbamate (400 mg, 1 Eq, 673 μmol). HATU (307 mg, 1.2 Eq, 808 μmol), N-(((9H-fluoren-9-yl)methoxy)carbonyl)-N-methylglycine (210 mg, 1 Eq, 673 μmol), diisopropylethylamine (DIEA) (1.13 g, 1.52 mL, 13.0 Eq, 8.75 mmol) and dimethylformamide (DMF) (5.0 mL). The reaction was stirred at 25° C. for 2 hours. The crude product was purified by MPLC with the following conditions: Column: SunFire prep OBD 19*150 mm 5 um; Mobile Phase A: Water (0.05% aqueous ammonia); Mobile Phase B: ACN; Gradient: 20% B to 98% B in 8 min; Flow rate: 60 mL/min; Wave Length: 254 nm. The collected fractions were concentrated under reduced pressure to provide (9H-fluoren-9-yl)methyl (2-((2-((4-(4-((tert-butoxycarbonyl)amino)piperidin-1-yl)-3-(6-chloro-1H-benzo[d]imidazol-2-yl)-5-(3-fluoro-5-methylphenyl)pyridin-2-yl)amino)ethyl)amino)-2-oxoethyl)(methyl)carbamate (360 mg, 406 μmol, 60.3%); (M+H)⁺=807.5.

Step B-2, preparation of tert-butyl (1-(3-(6-chloro-1H-benzo[d]imidazol-2-yl)-5-(3-fluoro-5-methylphenyl)-2-((2-(2-(methylamino)acetamido)ethyl)amino)pyridin-4-yl)piperidin-4-yl)carbamate: Into a 8-mL vial was placed a mixture of (9H-fluoren-9-yl)methyl (2-((2-((4-(4-((tert-butoxycarbonyl)amino)piperidin-1-yl)-3-(6-chloro-1H-benzo[d]imidazol-2-yl)-5-(3-fluoro-5-methylphenyl)pyridin-2-yl)amino)ethyl)amino)-2-oxoethyl)(methyl)carbamate (360 mg, 1 Eq, 406 μmol), 1,8-diazabicyclo[5.4.0]undec-7-ene (DBU)(180 mg, 2.91 Eq, 1.18 mmol,) and DMF (3.0 mL). The reaction was stirred at 25° C. for 2 hours. The crude product was purified by MPLC with the following conditions: Column: SunFire prep OBD 19*150 mm 5 um; Mobile Phase A: Water (0.05% aqueous ammonia); Mobile Phase B: ACN; Gradient: 20% B to 98% B in 8 min; Flow rate: 60 mL/min; Wave Length: 254 nm. The collected fractions were concentrated under reduced pressure to provide tert-butyl (1-(3-(6-chloro-1H-benzo[d]imidazol-2-yl)-5-(3-fluoro-5-methylphenyl)-2-((2-(2-(methylamino)acetamido)ethyl)amino)pyridin-4-yl)piperidin-4-yl)carbamate (300 mg, 0.36 mmol, 89%, 80% Purity); (M+H)⁺=665.4.

Step B-3, preparation of 3-(3-(2-((2-((4-(4-((tert-butoxycarbonyl)amino)piperidin-1-yl)-3-(6-chloro-1H-benzo[d]imidazol-2-yl)-5-(3-fluoro-5-methylphenyl)pyridin-2-yl)amino)ethyl)amino)-2-oxoethyl)(methyl)amino)-3-oxopropoxy)propanoic acid: Into a 8-mL vial was placed a mixture of tert-butyl (1-(3-(6-chloro-1H-benzo[d]imidazol-2-yl)-5-(3-fluoro-5-methylphenyl)-2-((2-(2-(methylamino)acetamido)ethyl)amino)pyridin-4-yl)piperidin-4-yl)carbamate (230 mg, 1 Eq, 346 μmol), HATU (150 mg, 1.14 Eq, 394 μmol), 3,3′-oxydipropionic acid (60 mg, 1.1 Eq, 0.37 mmol), DIEA (135 mg, 182 μL, 3.02 Eq, 1.04 mmol) and DMF (3.0 mL). The reaction stirred at 25° C. for 2 hours. The crude product was purified by MPLC with the following conditions: Column: SunFire prep OBD 19*150 mm 5 um; Mobile Phase A: Water (0.05% aqueous ammonia); Mobile Phase B: ACN; Gradient: 20% B to 98% B in 8 min; Flow rate: 60 mL/min; Wave Length: 254 nm. The collected fractions were concentrated under reduced pressure to provide 3-(3-((2-((2-((4-(4-((tert-butoxy carbonyl)amino)piperidin-1-yl)-3-(6-chloro-1H-benzo[d]imidazol-2-yl)-5-(3-fluoro-5-methylphenyl)pyridin-2-yl)amino)ethyl)amino)-2-oxoethyl)(methyl)amino)-3-oxopropoxy)propanoic acid (250 mg, 309 μmol, 89.3%); (M+H)⁺=809.6.

Step B-4, preparation of (2S,3R,4S,5S,6S)-2-(2-((2S,5S)-19-((4-(4-((tert-butoxycarbonyl)amino)piperidin-1-yl)-3-(6-chloro-1H-benzo[d]imidazol-2-yl)-5-(3-fluoro-5-methylphenyl)pyridin-2-yl)amino)-5-isopropyl-2,14-dimethyl-4,7,13,16-tetraoxo-10-oxa-3,6,14,17-tetraazanonadecanamido)-5-((5S,8S,11S,12R)-11-((S)-sec-butyl)-12-(2-((S)-2-((1R,2R)-3-(((1S,2R)-1-hydroxy-1-phenylpropan-2-yl)amino)-1-methoxy-2-methyl-3-oxopropyl)pyrrolidin-1-yl)-2-oxoethyl)-5,8-diisopropyl-4,10-dimethyl-3,6,9-trioxo-2,13-dioxa-4,7,10-triazatetradecyl)phenoxy)-6-(methoxycarbonyl)tetrahydro-2H-pyran-3,4,5-triyl triacetate: Into a 8-mL vial was placed a mixture of 3-(3-((2-((2-((4-(4-((tert-butoxy carbonyl)amino)piperidin-1-yl)-3-(6-chloro-1H-benzo[d]imidazol-2-yl)-5-(3-fluoro-5-methylphenyl)pyridin-2-yl)amino)ethyl)amino)-2-oxoethyl)methyl)amino)-3-oxopropoxy)propanoicacid (60 mg, 1.0 Eq, 74 μmol), N-methylmorpholine (NMM)(8 mg, 1 Eq, 0.08 mmol), pentafluorophenyl diphenylphosphinate (FDPP) (85 mg, 3.0 Eq. 0.22 mmol), (2S,3R,4S,5S,6S)-2-(2-((S)-2-((S)-2-amino-3-methylbutanamido)propanamido)-5-((5S,8S,11S,12R)-11-((S)-sec-butyl)-12-(2-((S)-2-((1R,2R)-3-(((1S,2R)-1-hydroxy-1-phenylpropan-2-yl)amino)-1-methoxy-2-methyl-3-oxopropyl)pyrrolidin-1-yl)-2-oxoethyl)-5,8-diisopropyl-4,10-dimethyl-3,6,9-trioxo-2,13-dioxa-4,7,10-triazatetradecyl)phenoxy)-6-(methoxycarbonyl)tetrahydro-2H-pyran-3,4,5-triyl triacetate (100 mg, 1 Eq, 73.0 μmol) and DMF (1.0 mL). The reaction stirred at 25° C. for 2 hours. The crude product was purified by MPLC with the following conditions: Column: SunFire prep OBD 19*150 mm 5 um; Mobile Phase A: Water (0.05% aqueous ammonia); Mobile Phase B: ACN; Gradient: 20% B to 98% B in 8 min; Flow rate: 60 mL/min; Wave Length: 254 nm. The collected fractions were concentrated under reduced pressure to provide (2S,3R,4S,5S,6S)-2-(2-((2S,5S)-19-((4-(4-((tert-butoxy carbonyl)amino)piperidin-1-yl)-3-(6-chloro-1H-benzo[d]imidazol-2-yl)-5-(3-fluoro-5-methylphenyl)pyridin-2-yl)amino)-5-isopropyl-2,14-dimethyl-4,7,13,16-tetraoxo-10-oxa-3,6,14,17-tetraazanonadecanamido)-5-((5S,8S,11S,12R)-11-((S)-sec-butyl)-12-(2-((S)-2-((1R,2R)-3-(((I S,2R)-1-hydroxy-1-phenylpropan-2-yl)amino)-1-methoxy-2-methyl-3-oxopropyl)pyrrolidin-1-yl)-2-oxoethyl)-5,8-diisopropyl-4,10-dimethyl-3,6,9-trioxo-2,13-dioxa-4,7,10-triazatetradecyl)phenoxy)-6-(methoxycarbonyl)tetrahydro-2H-pyran-3,4,5-triyl triacetate (80 mg, 37 μmol, 51%); (M/2+H)⁺=1081.4.

Step B-5, preparation of (2S,3S,4S,5R,6S)-3,4,5-triacetoxy-6-(2-((2S,5S)-19-((4-(4-((tert-butoxycarbonyl)amino)piperidin-1-yl)-3-(6-chloro-1H-benzo[d]imidazol-2-yl)-5-(3-fluoro-5-methylphenyl)pyridin-2-yl)amino)-5-isopropyl-2,14-dimethyl-4,7,13,16-tetraoxo-10-oxa-3,6,14,17-tetraazanonadecanamido)-5-((5S,8S,11S,12R)-11-((S)-sec-butyl)-12-(2-((S)-2-((1R,2R)-3-(((1S,2R)-1-hydroxy-1-phenylpropan-2-yl)amino)-1-methoxy-2-methyl-3-oxopropyl)pyrrolidin-1-yl)-2-oxoethyl)-5,8-diisopropyl-4,10-dimethyl-3,6,9-trioxo-2,13-dioxa-4,7,10-triazatetradecyl)phenoxy)tetrahydro-2H-pyran-2-carboxylic acid: Into a 8-mL vial was placed a mixture of (2S,3R,4S,5S,6S)-2-(2-((2S,5S)-19-((4-(4-((tert-butoxycarbonyl)amino)piperidin-1-yl)-3-(6-chloro-1H-benzo[d]imidazol-2-yl)-5-(3-fluoro-5-methylphenyl)pyridin-2-yl)amino)-5-isopropyl-2,14-dimethyl-4,7,13,16-tetraoxo-10-oxa-3,6,14,17-tetraazanonadecanamido)-5-((5S,8S,11S,12R)-11-((S)-sec-butyl)-12-(2-((S)-2-((1R,2R)-3-(((1S,2R)-1-hydroxy-1-phenylpropan-2-yl)amino)-1-methoxy-2-methyl-3-oxopropyl)pyrrolidin-1-yl)-2-oxoethyl)-5,8-diisopropyl-4,10-dimethyl-3,6,9-trioxo-2,13-dioxa-4,7,10-triazatetradecyl)phenoxy)-6-(methoxycarbonyl)tetrahydro-2H-pyran-3,4,5-triyl triacetate (70 mg, 1 Eq, 32 μmol), trimethyltinhydroxide (35 mg, 6.0 Eq, 0.19 mmol), and dichloroethane (DCE) (1.0 mL). The reaction stirred at 50° C. for 4 hours. The crude product was purified by MPLC with the following conditions: Column: SunFire prep OBD 19*150 mm 5 um; Mobile Phase A: Water (0.05% aqueous ammonia); Mobile Phase B: ACN; Gradient: 20% B to 98% B in 8 min; Flow rate: 60 mL/min; Wave Length: 254 nm. The collected fractions were concentrated under reduced pressure to provide (2S,3S,4S,5R,6S)-3,4,5-triacetoxy-6-(2-((2S,5S)-19-((4-(4-((tert-butoxycarbonyl)amino)piperidin-1-yl)-3-(6-chloro-1H-benzo[d]imidazol-2-yl)-5-(3-fluoro-5-methylphenyl)pyridin-2-yl)amino)-5-isopropyl-2,14-dimethyl-4,7,13,16-tetraoxo-10-oxa-3,6,14,17-tetraazmnonadecanamido)-5-((5S,8S,11S,12R)-1-((S)-sec-butyl)-12-(2-((S)-2-((1R,2R)-3-(((1S,2R)-1-hydroxy-1-phenylpropan-2-yl)amino)-1-methoxy-2-methyl-3-oxopropyl)pyrrolidin-1-yl)-2-oxoethyl)-5,8-diisopropyl-4,10-dimethyl-3,6,9-trioxo-2,13-dioxa-4,7,10-triazatetradecyl)phenoxy)tetrahydro-2H-pyran-2-carboxylic acid (40 mg, 19 μmol, 58%); (M/2+H)⁺=1074.5.

Step B-6, preparation of (2S,3S,4S,5R,6S)-2-((2-aminoethyl)carbamoyl)-6-(2-((2S,5S)-19-((4-(4-((tert-butoxycarbonyl)amino)piperidin-1-yl)-3-(6-chloro-1H-benzo[d]imidazol-2-yl)-5-(3-fluoro-5-methylphenyl)pyridin-2-yl)amino)-5-isopropyl-2,14-dimethyl-4,7,13,16-tetraoxo-10-oxa-3,6,14,17-tetraazanonadecanamido)-5-((5S,8S,11S,12R)-11-((S)-sec-butyl)-12-(2-((S)-2-((1R,2R)-3-(((1S,2R)-1-hydroxy-1-phenylpropan-2-yl)amino)-1-methoxy-2-methyl-3-oxopropyl)pyrrolidin-1-yl)-2-oxoethyl)-5,8-diisopropyl-4,10-dimethyl-3,6,9-trioxo-2,13-dioxa-4,7,10-triazatetradecyl)phenoxy)tetrahydro-2H-pyran-3,4,5-triyl triacetate: Into a 8-mL vial was placed a mixture of (2S,3S,4S,5R,6S)-3,4,5-triacetoxy-6-(2-((2S,5S)-19-((4-(4-((tert-butoxycarbonyl)amino)piperidin-1-yl)-3-(6-chloro-1H-benzo[d]imidazol-2-yl)-5-(3-fluoro-5-methylphenyl)pyridin-2-yl)amino)-5-isopropyl-2,14-dimethyl-4,7,13,16-tetraoxo-10-oxa-3,6,14,17-tetraazanonadecanamido)-5-((5S,8S,11S,12R)-11-((S)-sec-butyl)-12-(2-((S)-2-((1R,2R)-3-(((1S,2R)-1-hydroxy-1-phenylpropan-2-yl)amino)-1-methoxy-2-methyl-3-oxopropyl)pyrrolidin-1-yl)-2-oxoethyl)-5,8-diisopropyl-4,10-dimethyl-3,6,9-trioxo-2,13-dioxa-4,7,10-triazatetradecyl)phenoxy)tetrahydro-2H-pyran-2-carboxylic acid (40 mg, 1 Eq, 19 μmol), ethane-1,2-diamine (1.2 mg, 1.1 Eq, 20 μmol), HBTU (7.8 mg, 1.1 Eq, 20 μmol). DIEA (7.2 mg, 9.7 μL, 3.0 Eq, 56 μmol) and DMF (1.0 mL). The reaction stirred at 25° C. for 2 hours. The crude product was purified by MPLC with the following conditions: Column: SunFire prep OBD 19*150 mm 5 um; Mobile Phase A: Water (0.05% aqueous ammonia); Mobile Phase B: ACN; Gradient: 20% B to 98% B in 8 min; Flow rate: 60 mL/min; Wave Length: 254 nm. The collected fractions were concentrated under reduced pressure to provide (2S,3S,4S,5R,6S)-2-((2-aminoethyl)carbamoyl)-6-(2-((2S,5 S)-19-((4-(4-((tert-butoxycarbonyl)amino)piperidin-1-yl)-3-(6-chloro-1H-benzo[d]imidazol-2-yl)-5-(3-fluoro-5-methylphenyl)pyridin-2-yl)amino)-5-isopropyl-2,14-dimethyl-4,7,13,16-tetraoxo-10-oxa-3,6,14,17-tetraazanonadecanamido)-5-((5S,8S,11S,12R)-11-((S)-sec-butyl)-12-(2-((S)-2-((1R,2R)-3-(((1S,2R)-1-hydroxy-1-phenylpropan-2-yl)amino)-1-methoxy-2-methyl-3-oxopropyl)pyrrolidin-1-yl)-2-oxoethyl)-5,8-diisopropyl-4,10-dimethyl-3,6,9-trioxo-2,13-dioxa-4,7,10-triazatetradecyl)phenoxy)tetrahy dro-2H-pyran-3,4,5-triyl triacetate (20 mg, 9.1 μmol, 49%); (M/2+H)⁺=1053.2.

Step B-7, preparation of 3-(((2S,3R,4S,5S,6S)-6-((2-aminoethyl)carbamoyl)-3,4,5-trihydroxytetrahydro-2H-pyran-2-yl)oxy)-4-((2S,5S)-19-((4-(4-((tert-butoxycarbonyl)amino)piperidin-1-yl)-3-(6-chloro-1H-benzo[d]imidazol-2-yl)-5-(3-fluoro-5-methylphenyl)pyridin-2-yl)amino)-5-isopropyl-2,14-dimethyl-4,7,13,16-tetraoxo-10-oxa-3,6,14,17-tetraazanonadecanamido)benzyl((S)-1-(((S)-1-(((3R,4S,5S)-1-((S)-2-((1R,2R)-3-(((1S,2R)-1-hydroxy-1-phenylpropan-2-yl)amino)-1-methoxy-2-methyl-3-oxopropyl)pyrrolidin-1-yl)-3-methoxy-5-methyl-1-oxoheptan-4-yl)(methyl)amino)-3-methyl-1-oxobutan-2-yl)amino)-3-methyl-1-oxobutan-2-yl)(methyl)carbamate: Into a 8-mL vial was placed a mixture of (2S,3S,4S,5R,6S)-2-((2-aminoethyl)carbamoyl)-6-(2-((2S,5S)-19-((4-(4-((tert-butoxy carbonyl)amino)piperidin-1-yl)-3-(6-chloro-1H-benzo[d]imidazol-2-yl)-5-(3-fluoro-5-methylphenyl)pyridin-2-yl)amino)-5-isopropyl-2,14-dimethyl-4,7,13,16-tetraoxo-10-oxa-3,6,14,17-tetraazanonadecanamido)-5-((5S,8S,11S,12R)-11-((S)-sec-butyl)-12-(2-((S)-2-((1R,2R)-3-(((1S,2R)-1-hydroxy-1-phenylpropan-2-yl)amino)-1-methoxy-2-methyl-3-oxopropyl)pyrrolidin-1-yl)-2-oxoethyl)-5,8-diisopropyl-4,10-dimethyl-3,6,9-trioxo-2,13-dioxa-4,7,10-triazatetradecyl)phenoxy)tetrahydro-2H-pyran-3,4,5-triyl triacetate (20 mg, 1 Eq, 9.1 μmol), LiOH (1.3 mg, 5.9 Eq, 54 μmol), H₂O (0.3 mL), and MeOH (0.3 mL). The reaction stirred at 0° C. for 30 minutes. The crude product was purified by Prep-HPLC with the following conditions: Column: SunFire prep OBD 19*150 mm 5 um; Mobile Phase A: Water (0.05% formic acid); Mobile Phase B: ACN; Gradient: 20% B to 98% B in 8 min; Flow rate: 60 mL/min; Wave Length: 254 nm. The collected fractions were dried by lyophilization to provide 3-(((2S,3R,4S,5S,6S)-6-((2-aminoethyl)carbamoyl)-3,4,5-trihydroxy tetrahydro-2H-pyran-2-yl)oxy)-4-((2S,5S)-19-((4-(4-((tert-butoxy carbonyl)amino)piperidin-1-yl)-3-(6-chloro-1H-benzo[d]imidazol-2-yl)-5-(3-fluoro-5-methylphenyl)pyridin-2-yl)amino)-5-isopropyl-2,14-dimethyl-4,7,13,16-tetraoxo-10-oxa-3,6,14,17-tetraazanonadecanamido)benzyl((S)-1-(((S)-1-(((3R,4S,5S)-1-((S)-2-((1R,2R)-3-(((S,2R)-1-hydroxy-1-phenylpropan-2-yl)amino)-1-methoxy-2-methyl-3-oxopropyl)pyrrolidin-1-yl)-3-methoxy-5-methyl-1-oxoheptan-4-yl)(methyl)amino)-3-methyl-1-oxobutan-2-yl)amino)-3-methyl-1-oxobutan-2-yl)(methyl)carbamate (15 mg, 7.3 μmol, 80%); (M+H)⁺=1032.2.

Step B-8, preparation of 1-(S)-1-1N-(S)-1-{[(1S,2R)-4-[(S)-2-{(1R,2R)-2-[N-(1R,2S)-2-hydroxy-1-methyl-2-phenylethylcarbamoyl]-1-methoxypropyl}-1-pyrrolidinyl]-1-[(S)-1-methylpropyl]-2-methoxy-4-oxobutyl]-N-methylcarbamoyl}-2-methylpropylcarbamoyl]-2-methylpropyl 1-methyl {3-[(2S,3R,4S,5S,6S)-6-(N-2-aminoethylcarbamoyl)-3,4,5-trihydroxytetrahydro-2H-pyran-2-yloxy]-4-[(S)-2-[(S)-2-(3-{2-[({N-2-[4-(4-amino-1-piperidyl)-3-(6-chloro-1H-1,3-benzimidazol-2-yl)-5-(3-fluoro-5-tolyl)-2-pyridylamino]ethylcarbamoyl}methyl)-N-methylcarbamoyl]ethoxy}propionylamino)-3-methylbutyrylamino]propionylamino]phenyl}methanecarbamate: Into a 8-mL vial was placed a mixture of 3-(((2S,3R,4S,5S,6S)-6-((2-aminoethyl)carbamoyl)-3,4,5-trihydroxytetrahydro-2H-pyran-2-yl)oxy)-4-((2S,5S)-19-((4-(4-((tert-butoxycarbonyl)amino)piperidin-1-yl)-3-(6-chloro-1H-benzo[d]imidazol-2-yl)-5-(3-fluoro-5-methylphenyl)pyridin-2-yl)amino)-5-isopropyl-2,14-dimethyl-4,7,13,16-tetraoxo-10-oxa-3,6,14,17-tetraazanonadecanamido)benzyl ((S)-1-(((S)-1-(((3R,4S,5S)-1-((S)-2-((1R,2R)-3-(((1S,2R)-1-hydroxy-1-phenylpropan-2-yl)amino)-1-methoxy-2-methyl-3-oxopropyl)pyrrolidin-1-yl)-3-methoxy-5-methyl-1-oxoheptan-4-yl)(methyl)amino)-3-methyl-1-oxobutan-2-yl)amino)-3-methyl-1-oxobutan-2-yl)(methyl)carbamate (15 mg, 1 Eq, 7.3 μmol) and DCM (0.3 mL), to which was added trifluoroacetic acid (0.1 mL). The reaction mixture was stirred at 0° C. for 2 hours. Then the pH was adjusted to ˜7 with DIEA dropwise at 0° C. The mixture was concentrated under reduced pressure. The crude product was purified by Prep-HPLC with the following conditions: Column: SunFireprep OBD 19*150 mm 5 um; Mobile Phase A: Water (0.05% trifluoroacetic acid); Mobile Phase B: ACN; Gradient: 25% B to 65% B in 8 min; Flowrate: 20 mL/min; Wave Length: 220 nm. The collected fractions were dried by lyophilization to provide 1-(S)-1-[N—(S)-1-{[(1S,2R)-4-[(S)-2{(1R,2R)-2-[N-(1R,2S)-2-hydroxy-1-methyl-2-phenylethylcarbamoyl]-1-methoxypropyl}-1-pyrrolidinyl]-1-[(S)-1-methylpropyl]-2-methoxy-4-oxobutyl]-N-methylcarbamoyl}-2-methylpropylcarbamoyl]-2-methylpropyl 1-methyl {3-[(2S,3R,4S,5S,6S)-6-(N-2-aminoethylcarbamoyl)-3,4,5-trihydroxytetrahydro-2H-pyran-2-yloxy]-4-[(S)-2-[(S)-2-(3-{2-[({N-2-[4-(4-amino-1-piperidyl)-3-(6-chloro-1H-1,3-benzimidazol-2-yl)-5-(3-fluoro-5-tolyl)-2-pyridylamino]ethylcarbamoyl}methyl)-N-methylcarbamoyl]ethoxy}propionylamino)-3-methylbutyrylamino]propionylamino]phenyl}methanecarbamate (6.0 mg, 2.6 μmol, 36%); (M+H)⁺=1961.9.

The following conjugates were prepared similarly to Example B with appropriate substituting reagents and substrates at different steps and they may require additional functional group modifications via well-known chemistry with appropriate reagents.

| | MS |
|---|---|
| Compound | (M + H)⁺ |
| 1-(S)-1-[N-(S)-1-{[(1S,2R)-4-[(S)-2-{(1R,2R)-2-[N-(1R,2S)-2-hydroxy-1-methyl-2-phenylethylcarbamoyl]-1-methoxypropyl}- | 1890.9 |
| 1-pyrrolidinyl]-1-[(S)-1-methylpropyl]-2-methoxy-4-oxobutyl]-N-methylcarbamoyl}-2-methylpropylcarbamoyl]-2- | |
| methylpropyl 1-methyl {3-[(2S,3R,4S,5S,6S)-6-(N-2-aminoethylcarbamoyl)-3,4,5-trihydroxytetrahydro-2H-pyran-2-yloxy]-4- | |
| [(S)-2-[(S)-2-[3-(2-{N-2-[4-(4-amino-1-piperidyl)-3-(6-chloro-1H-1,3-benzimidazol-2-yl)-5-(3-fluoro-5-toly])-2- | |
| pyridylamino]ethylcarbamoyl}ethoxy)propionylamino]-3-methylbutyrylamino]propionylamino]phenyl}methanecarbamate | |
| 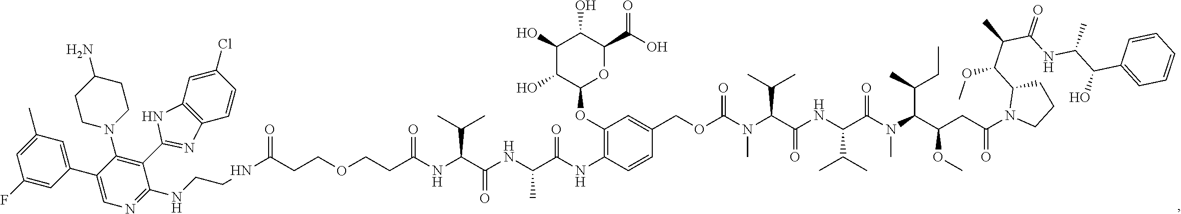 [SMILES: CC[C@H](C)[C@@H]([C@@H](CC(=O)N1CCC[C@H]1[C@H](OC)[C@@H](C)C(=O)N[C@H](C)[C@@H](O)c1ccccc1)OC)N(C)C(=O)[C@@H](NC(=O)[C@H](C(C)C)N(C)C(=O)OCc1ccc(NC(=O)[C@H](C)NC(=O)[C@@H](NC(=O)CCOCCC(=O)NCCNc2ncc(-c3cc(C)cc(F)c3)c(N3CCC(N)CC3)c2-c2nc3ccc(Cl)cc3[nH]2)C(C)C)c(O[C@@H]2O[C@H](C(=O)O)[C@@H](O)[C@H](O)[C@H]2O)c1)C(C)C] | |
| (Compound 32) | |
| [(2S,3S,4S,5R,6S)-6-[5-({[(S)-1-[N-(S)-1-{[(1S,2R)-4-[(S)-2-{(1R,2R)-2-[N-(1R,2S)-2-hydroxy-1-methyl-2- | 1905.9 |
| phenylethylcarbamoyl]-1-methoxypropyl}-1-pyrrolidinyl]-1-[(S)-1-methylpropyl]-2-methoxy-4-oxobutyl]-N- | |
| methylcarbamoyl}-2-methylpropylcarbamoyl]-2-methylpropyl](methyl)(aminocarbonyloxy)}methyl)-2-[(S)-2-[(S)-2-13-(2-{N- | |
| 2-[4-(4-amino-1-piperidyl)-3-(6-chloro-1H-1,3-benzimidazol-2-yl)-5-(3-fluoro-5-tolyl)-2- | |
| pyridylamino]ethylcarbamoyl}ethoxy)propionylamino]-3-methylbutyrylamino]propionylamino]phenoxy]-3,4,5- | |
| trihydroxytetrahydro-2H-pyran-2-ylcarbonylamino]acetic acid | |
| 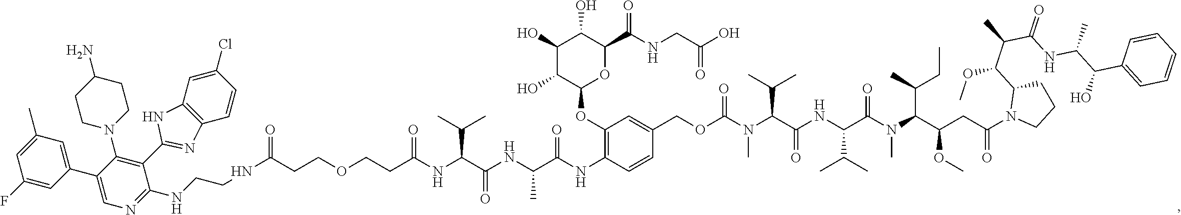 [SMILES: CC[C@H](C)[C@@H]([C@@H](CC(=O)N1CCC[C@H]1[C@H](OC)[C@@H](C)C(=O)N[C@H](C)[C@@H](O)c1ccccc1)OC)N(C)C(=O)[C@@H](NC(=O)[C@H](C(C)C)N(C)C(=O)OCc1ccc(NC(=O)[C@H](C)NC(=O)[C@@H](NC(=O)CCOCCC(=O)NCCNc2ncc(-c3cc(C)cc(F)c3)c(N3CCC(N)CC3)c2-c2nc3ccc(Cl)cc3[nH]2)C(C)C)c(O[C@@H]2O[C@H](C(=O)NCC(=O)O)[C@@H](O)[C@H](O)[C@H]2O)c1)C(C)C] | |
| (Compound 33) | |
| 1-(S)-1-[N-(S)-1-{[(1S,2R)-4-[(S)-2-{(1R,2R)-2-[N-(1R,2S)-2-hydroxy-1-methyl-2-phenylethylcarbamoyl]-1-methoxypropyl}- | 1847.9 |
| 1-pyrrolidinyl]-1-[(S)-1-methylpropyl]-2-methoxy-4-oxobutyl]-N-methylcarbamoyl}-2-methylpropylcarbamoyl]-2- | |
| methylpropyl 1-methyl {3-[(2S,3R,4S,5S,6S)-6-carbamoyl-3,4,5-trihydroxytetrahydro-2H-pyran-2-yloxy]-4-[(S)-2-[(S)-2-[3-(2- | |
| {N-2-[4-(4-amino-1-piperidyl)-3-(6-chloro-1H-1,3-benzimidazol-2-yl)-5-(3-fluoro-5-tolyl)-2- | |
| pyridylamino]ethylcarbamoyl}ethoxy)propionylamino]-3-methylbutyrylamino]propionylamino]phenyl}methanecarbamate | |
| 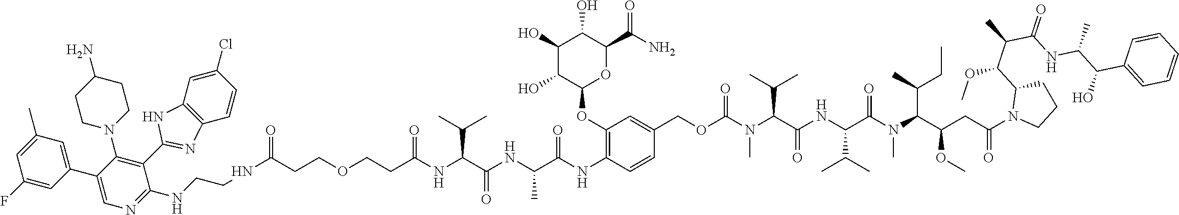 [SMILES: CC[C@H](C)[C@@H]([C@@H](CC(=O)N1CCC[C@H]1[C@H](OC)[C@@H](C)C(=O)N[C@H](C)[C@@H](O)c1ccccc1)OC)N(C)C(=O)[C@@H](NC(=O)[C@H](C(C)C)N(C)C(=O)OCc1ccc(NC(=O)[C@H](C)NC(=O)[C@@H](NC(=O)CCOCCC(=O)NCCNc2ncc(-c3cc(C)cc(F)c3)c(N3CCC(N)CC3)c2-c2nc3ccc(Cl)cc3[nH]2)C(C)C)c(O[C@@H]2O[C@H](C(N)=O)[C@@H](O)[C@H](O)[C@H]2O)c1)C(C)C] | |
| (Compound 34) | |
| [(2S,3S,4S,5R,6S)-6-[5-({[(S)-1-[N-(S)-1-{[(1S,2R)-4-[(S)-2-{(1R,2R)-2-[N-(1R,2S)-2-hydroxy-1-methyl-2- | 2078.0 |
| phenylethylcarbamoyl]-1-methoxypropyl}-1-pyrrolidinyl]-1-[(S)-1-methylpropyl]-2-methoxy-4-oxobutyl]-N- | |
| methylcarbamoyl}-2-methylpropylcarbamoyl]-2-methylpropyl](methyl)(aminocarbonyloxy)}methyl)-2-[(S)-2-[(S)-2-[(R)-3-[N- | |
| 2-(2-{N-2-[4-(4-amino-1-piperidyl)-3-(6-chloro-1H-1,3-benzimidazol-2-yl)-5-(3-fluoro-5-tolyl)-2- | |
| pyridylamino]ethylcarbamoyl}ethoxy)ethylcarbamoyl]-3-aminopropionylamino]-3-methylbutyrylamino]-5- | |
| ureidovalerylamino]phenoxy]-3,4,5-trihydroxytetrahydro-2H-pyran-2-ylcarbonylamino]acetic acid | |
| 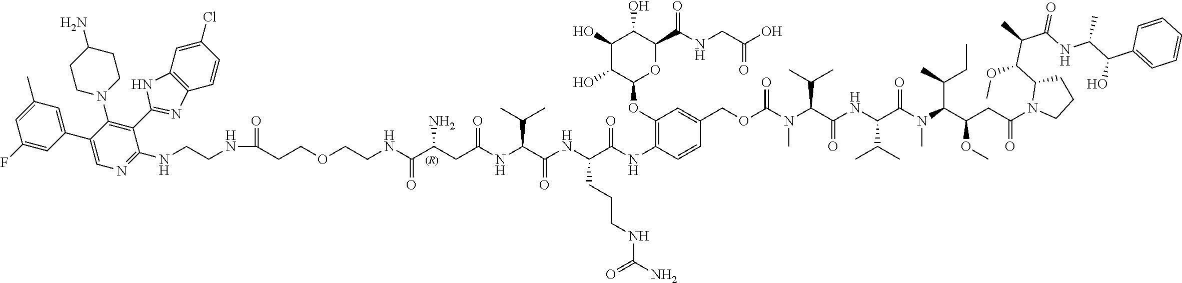 | |
| (Compound 35) | |
| [(2S,3S,4S,5R,6S)-6-[5-({[(S)-1-[N-(S)-1-{[(1S,2R)-4-[(S)-2-{(1R,2R)-2-[N-(1R,2S)-2-hydroxy-1-methyl-2- | 1992.0 |
| phenylethylcarbamoyl]-1-methoxypropyl}-1-pyrrolidinyl]-1-[(S)-1-methylpropyl]-2-methoxy-4-oxobutyl]-N- | |
| methylcarbamoyl}-2-methylpropylcarbamoyl]-2-methylpropyl](methyl)(aminocarbonyloxy)}methyl)-2-[(S)-2-[(S)-2-[3-(2-{N- | |
| 2-[4-(4-amino-1-piperidyl)-3-(6-chloro-1H-1,3-benzimidazol-2-yl)-5-(3-fluoro-5-tolyl)-2- | |
| pyridylamino]ethylcarbamoyl}ethoxy)propionylamino]-3-methylbutyrylamino]-5-ureidovalerylamino]phenoxy]-3,4,5- | |
| trihydroxytetrahydro-2H-pyran-2-ylcarbonylamino]acetic acid | |
| 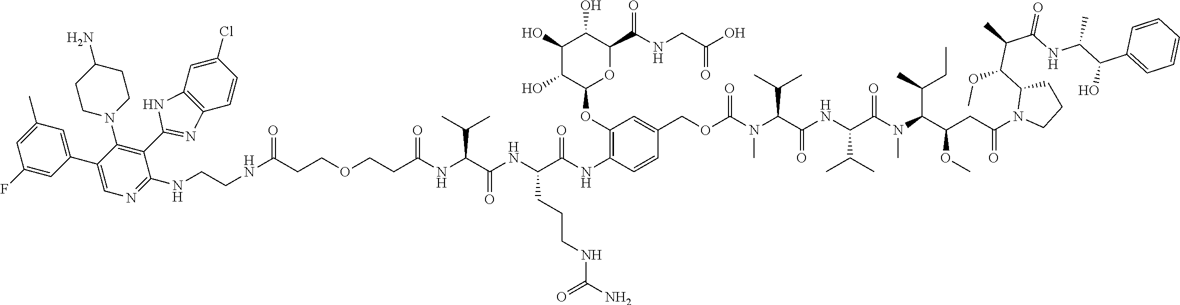 [SMILES: CC[C@H](C)[C@@H]([C@@H](CC(=O)N1CCC[C@H]1[C@H](OC)[C@@H](C)C(=O)N[C@H](C)[C@@H](O)c1ccccc1)OC)N(C)C(=O)[C@@H](NC(=O)[C@H](C(C)C)N(C)C(=O)OCc1ccc(NC(=O)[C@H](CCCNC(N)=O)NC(=O)[C@@H](NC(=O)CCOCCC(=O)NCCNc2ncc(-c3cc(C)cc(F)c3)c(N3CCC(N)CC3)c2-c2nc3ccc(Cl)cc3[nH]2)C(C)C)c(O[C@@H]2O[C@H](C(=O)NCC(=O)O)[C@@H](O)[C@H](O)[C@H]2O)c1)C(C)C] | |
| (Compound 36) | |
| [(2S,3S,4S,5R,6S)-6-[5-({[(S)-1-[N-(S)-1-{[(1S,2R)-4-[(S)-2-{(1R,2R)-2-[N-(1R,2S)-2-hydroxy-1-methyl-2- | 2063.0 |
| phenylethylcarbamoyl]-1-methoxypropyl}-1-pyrrolidinyl]-1-[(S)-1-methylpropyl]-2-methoxy-4-oxobutyl]-N- | |
| methylcarbamoyl}-2-methylpropylcarbamoyl]-2-methylpropyl](methyl)(aminocarbonyloxy)}methyl)-2-[(S)-2-[(S)-2-(3-{2- | |
| [({N-2-[4-(4-amino-1-piperidyl)-3-(6-chloro-1H-1,3-benzimidazol-2-yl)-5-(3-fluoro-5-tolyl)-2- | |
| pyridylamino]ethylcarbamoyl}methyl)-N-methylcarbamoyl]ethoxy}propionylamino)-3-methylbutyrylamino]-5- | |
| ureidovalerylamino]phenoxy]-3,4,5-trihydroxytetrahydro-2H-pyran-2-ylcarbonylamino]acetic acid | |
| 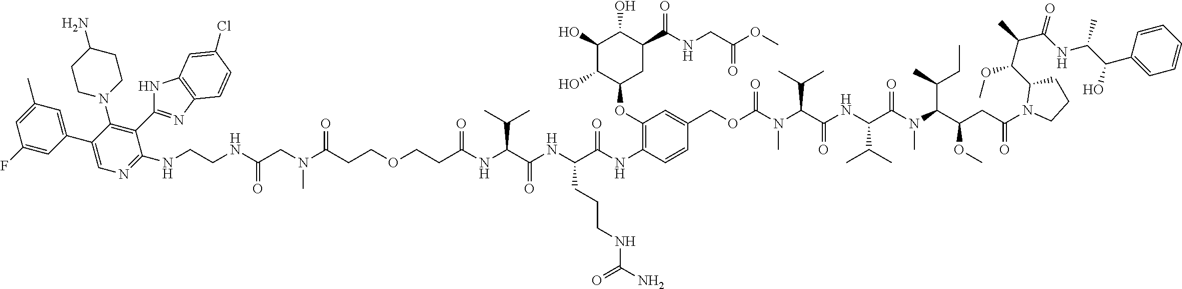 [SMILES: CC[C@H](C)[C@@H]([C@@H](CC(=O)N1CCC[C@H]1[C@H](OC)[C@@H](C)C(=O)N[C@H](C)[C@@H](O)c1ccccc1)OC)N(C)C(=O)[C@@H](NC(=O)[C@H](C(C)C)N(C)C(=O)OCc1ccc(NC(=O)[C@H](CCCNC(N)=O)NC(=O)[C@@H](NC(=O)CCOCCC(=O)N(C)CC(=O)NCCNc2ncc(-c3cc(C)cc(F)c3)c(N3CCC(N)CC3)c2-c2nc3ccc(Cl)cc3[nH]2)C(C)C)c(O[C@@H]2C[C@H](C(=O)NCC(=O)OC)[C@@H](O)[C@H](O)[C@H]2O)c1)C(C)C] | |
| (Compound 37) | |
| 1-(S)-1-[N-(S)-1-{[(1S,2R)-4-[(S)-2-{(1R,2R)-2-[N-(1R,2S)-2-hydroxy-1-methyl-2-phenylethylcarbamoyl]-1-methoxypropyl}- | 1934.0 |
| 1-pyrrolidinyl]-1-[(S)-1-methylpropyl]-2-methoxy-4-oxobutyl]-N-methylcarbamoyl}-2-methylpropylcarbamoyl]-2- | |
| methylpropyl 1-methyl {3-[(2S,3R,4S,5S,6S)-6-carbamoyl-3,4,5-trihydroxytetrahydro-2H-pyran-2-yloxy]-4-[(S)-2-[(S)-2-[3-(2- | |
| {N-2-[4-(4-amino-1-piperidyl)-3-(6-chloro-1H-1,3-benzimidazol-2-yl)-5-(3-fluoro-5-tolyl)-2- | |
| pyridylamino]ethylcarbamoyl}ethoxy)propionylamino]-3-methylbutyrylamino]-5- | |
| ureidovalerylamino]phenyl}methanecarbamate | |
| 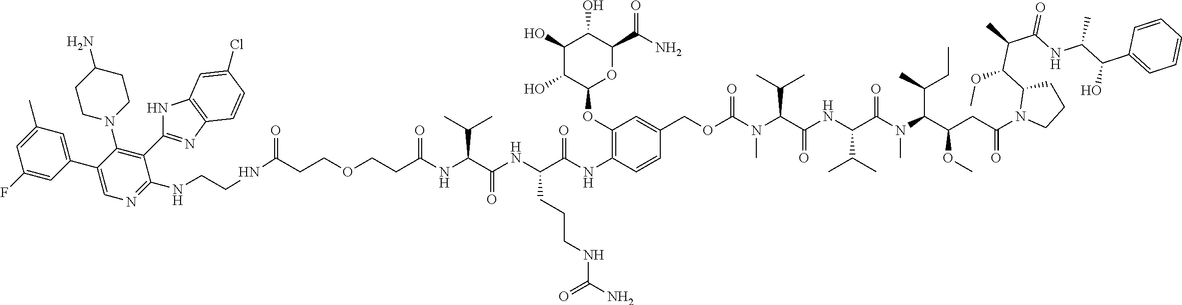 [SMILES: CC[C@H](C)[C@@H]([C@@H](CC(=O)N1CCC[C@H]1[C@H](OC)[C@@H](C)C(=O)N[C@H](C)[C@@H](O)c1ccccc1)OC)N(C)C(=O)[C@@H](NC(=O)[C@H](C(C)C)N(C)C(=O)OCc1ccc(NC(=O)[C@H](CCCNC(N)=O)NC(=O)[C@@H](NC(=O)CCOCCC(=O)NCCNc2ncc(-c3cc(C)cc(F)c3)c(N3CCC(N)CC3)c2-c2nc3ccc(Cl)cc3[nH]2)C(C)C)c(O[C@@H]2O[C@H](C(N)=O)[C@@H](O)[C@H](O)[C@H]2O)c1)C(C)C] | |
| (Compound 38) | |
| 1-(S)-1-[N-(S)-1-{[(1S,2R)-4-[(S)-2-{(1R,2R)-2-[N-(1R,2S)-2-hydroxy-1-methyl-2-phenylethylcarbamoyl]-1-methoxypropyl}- | 2005.0 |
| 1-pyrrolidinyl]-1-[(S)-1-methylpropyl]-2-methoxy-4-oxobutyl]-N-methylcarbamoyl}-2-methylpropylcarbamoyl]-2- | |
| methylpropyl 1-methyl {3-[(2S,3R,4S,5S,6S)-6-carbamoyl-3,4,5-trihydroxytetrahydro-2H-pyran-2-yloxy]-4-[(S)-2-[(S)-2-(3- | |
| (2-[({N-2-[4-(4-amino-1-piperidyl)-3-(6-chloro-1H-1,3-benzimidazol-2-yl)-5-(3-fluoro-5-tolyl)-2- | |
| pyridylamino]ethylcarbamoyl}methyl)-N-methylcarbamoyl]ethoxy}propionylamino)-3-methylbutyrylamino]-5- | |
| ureidovalerylamino]phenyl}methanecarbamate | |
| 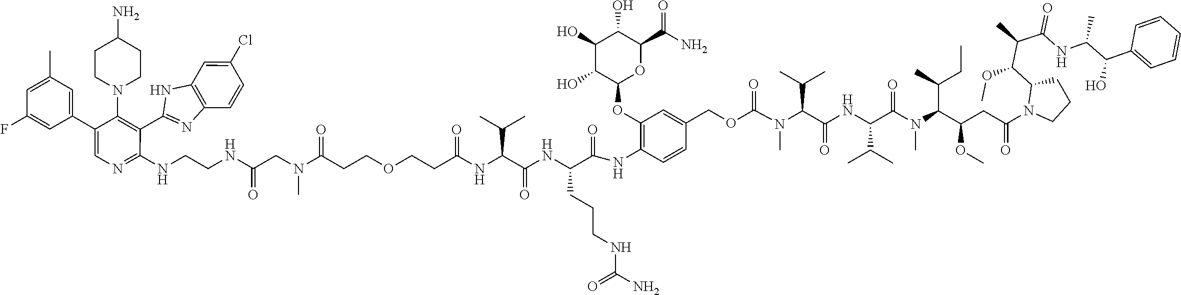 [SMILES: CC[C@H](C)[C@@H]([C@@H](CC(=O)N1CCC[C@H]1[C@H](OC)[C@@H](C)C(=O)N[C@H](C)[C@@H](O)c1ccccc1)OC)N(C)C(=O)[C@@H](NC(=O)[C@H](C(C)C)N(C)C(=O)OCc1ccc(NC(=O)[C@H](CCCNC(N)=O)NC(=O)[C@@H](NC(=O)CCOCCC(=O)N(C)CC(=O)NCCNc2ncc(-c3cc(C)cc(F)c3)c(N3CCC(N)CC3)c2-c2nc3ccc(Cl)cc3[nH]2)C(C)C)c(O[C@@H]2O[C@H](C(N)=O)[C@@H](O)[C@H](O)[C@H]2O)c1)C(C)C] | |
| (Compound 39) | |
| 1-(S)-1-[N-(S)-1-{[(1S,2R)-4-[(S)-2-{(1R,2R)-2-[N-(1R,2S)-2-hydroxy-1-methyl-2-phenylethylcarbamoyl]-1-methoxypropyl}- | 1977.0 |
| 1-pyrrolidinyl]-1-[(S)-1-methylpropyl]-2-methoxy-4-oxobutyl]-N-methylcarbamoyl}-2-methylpropylcarbamoyl]-2- | |
| methylpropyl 1-methyl {3-[(2S,3R,4S,5S,6S)-6-(N-2-aminoethylcarbamoyl)-3,4,5-trihydroxytetrahydro-2H-pyran-2-yloxy]-4- | |
| [(S)-2-[(S)-2-[3-(2-{N-2-[4-(4-amino-1-piperidyl)-3-(6-chloro-1H-1,3-benzimidazol-2-yl)-5-(3-fluoro-5-tolyl)-2- | |
| pyridylamino]ethylcarbamoyl}ethoxy)propionylamino]-3-methylbutyrylamino]-5- | |
| ureidovalerylamino]phenyl}methanecarbamate | |
| 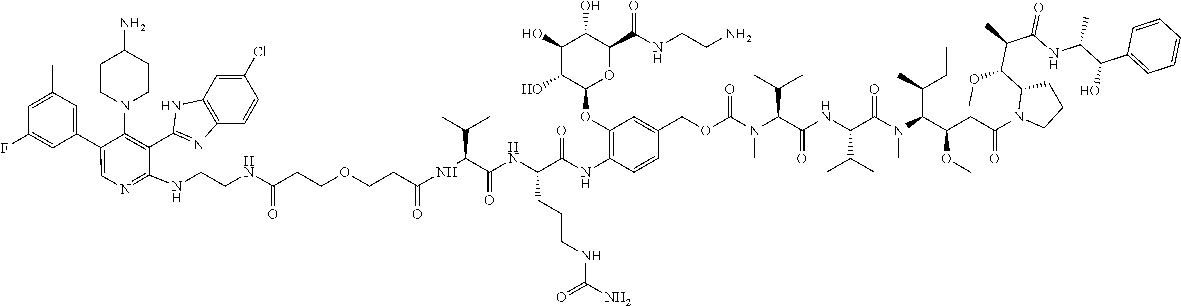 [SMILES: CC[C@H](C)[C@@H]([C@@H](CC(=O)N1CCC[C@H]1[C@H](OC)[C@@H](C)C(=O)N[C@H](C)[C@@H](O)c1ccccc1)OC)N(C)C(=O)[C@@H](NC(=O)[C@H](C(C)C)N(C)C(=O)OCc1ccc(NC(=O)[C@H](CCCNC(N)=O)NC(=O)[C@@H](NC(=O)CCOCCC(=O)NCCNc2ncc(-c3cc(C)cc(F)c3)c(N3CCC(N)CC3)c2-c2nc3ccc(Cl)cc3[nH]2)C(C)C)c(O[C@@H]2O[C@H](C(=O)NCCN)[C@@H](O)[C@H](O)[C@H]2O)c1)C(C)C] | |
| (Compound 40) | |
| (S)-2-[N-(S)-1-[N-(S)-1-[N-4-({[(S)-1-[N-(S)-1-{[(1S,2R)-4-[(S)-2-{(1R,2R)-2-[N-(1R,2S)-2-hydroxy-1-methyl-2- | 2042.0 |
| phenylethylcarbamoyl]-1-methoxypropyl}-1-pyrrolidinyl]-1-[(S)-1-methylpropyl]-2-methoxy-4-oxobutyl]-N- | |
| methylcarbamoyl}-2-methylpropylcarbamoyl]-2-methylpropyl](methyl)(aminocarbonyloxy)}methyl)-2-[(28,3R,4S,5S,6S)-6- | |
| (N-2-aminoethylcarbamoyl)-3,4,5-trihydroxytetrahydro-2H-pyran-2-yloxy]phenylcarbamoyl]ethylcarbamoyl]-2- | |
| methylpropylcarbamoyl]-2-[3-(2-{N-2-[4-(4-amino-1-piperidyl)-3-(6-chloro-1H-1,3-benzimidazol-2-yl)-5-(3-fluoro-5-tolyl)-2- | |
| pyridylamino]ethylcarbamoyl}ethoxy)propionylamino]ethanesulfonic acid | |
| 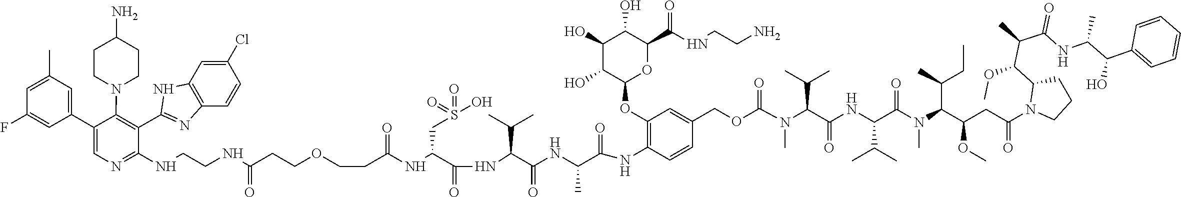 [SMILES: CC[C@H](C)[C@@H]([C@@H](CC(=O)N1CCC[C@H]1[C@H](OC)[C@@H](C)C(=O)N[C@H](C)[C@@H](O)c1ccccc1)OC)N(C)C(=O)[C@@H](NC(=O)[C@H](C(C)C)N(C)C(=O)OCc1ccc(NC(=O)[C@H](C)NC(=O)[C@@H](NC(=O)[C@@H](CS(=O)(=O)O)NC(=O)CCOCCC(=O)NCCNc2ncc(-c3cc(C)cc(F)c3)c(N3CCC(N)CC3)c2-c2nc3ccc(Cl)cc3[nH]2)C(C)C)c(O[C@@H]2O[C@H](C(=O)NCCN)[C@@H](O)[C@H](O)[C@H]2O)c1)C(C)C] | |
| (Compound 41) | |
| [(2S,3S,4S,5R,6S)-6-[5-({[(S)-1-[N-(S)-1-{[(1S,2R)-4-[(S)-2-{(1R,2R)-2-[N-(1R,2S)-2-hydroxy-1-methyl-2- | 1976.9 |
| phenylethylcarbamoyl]-1-methoxypropyl}-1-pyrrolidinyl]-1-[(S)-1-methylpropyl]-2-methoxy-4-oxobuty]]-N- | |
| methylcarbamoyl}-2-methylpropylcarbamoyl]-2-methylpropyl](methyl)(aminocarbonyloxy)}methyl)-2-[(S)-2-[(S)-2-(3-{2- | |
| [({N-2-[4-(4-amino-1-piperidyl)-3-(6-chloro-1H-1,3-benzimidazol-2-yl)-5-(3-fluoro-5-tolyl)-2- | |
| pyridylamino]ethylcarbamoyl}methyl)-N-methylcarbamoyl]ethoxy}propionylamino)-3- | |
| methylbutyrylamino]propionylamino]phenoxy]-3,4,5-trihydroxytetrahydro-2H-pyran-2-ylcarbonylamino]acetic acid | |
| 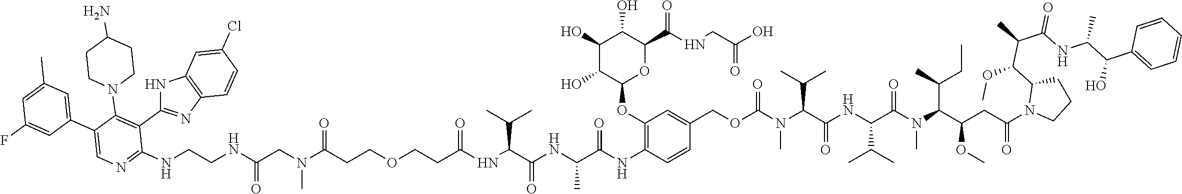 [SMILES: CC[C@H](C)[C@@H]([C@@H](CC(=O)N1CCC[C@H]1[C@H](OC)[C@@H](C)C(=O)N[C@H](C)[C@@H](O)c1ccccc1)OC)N(C)C(=O)[C@@H](NC(=O)[C@H](C(C)C)N(C)C(=O)OCc1ccc(NC(=O)[C@H](C)NC(=O)[C@@H](NC(=O)CCOCCC(=O)N(C)CC(=O)NCCNc2ncc(-c3cc(C)cc(F)c3)c(N3CCC(N)CC3)c2-c2nc3ccc(Cl)cc3[nH]2)C(C)C)c(O[C@@H]2O[C@H](C(=O)NCC(=O)O)[C@@H](O)[C@H](O)[C@H]2O)c1)C(C)C] | |
| (Compound 42) | |
| 1-(S)-1-[N-(S)-1-{[(1S,2R)-4-[(S)-2-{(1R,2R)-2-[N-(1R,2S)-2-hydroxy-1-methyl-2-phenylethylcarbamoyl]-1-methoxypropyl}- | 1976.0 |
| 1-pyrrolidinyl]-1-[(S)-1-methylpropyl]-2-methoxy-4-oxobutyl]-N-methylcarbamoyl}-2-methylpropylcarbamoyl]-2- | |
| methylpropyl 1-methyl {3-[(2S,3R,4S,5S,6S)-6-(N-2-aminoethylcarbamoyl)-3,4,5-trihydroxytetrahydro-2H-pyran-2-yloxy]-4- | |
| [(S)-2-[(S)-2-[3-(2-{N-2-[4-(4-amino-1-piperidyl)-3-(6-chloro-2-indoly])-5-(3-fluoro-5-tolyl)-2- | |
| pyridylamino]ethylcarbamoyl}ethoxy)propionylamino]-3-methylbutyrylamino]-5- | |
| ureidovalerylamino]phenyl}methanecarbamate | |
| 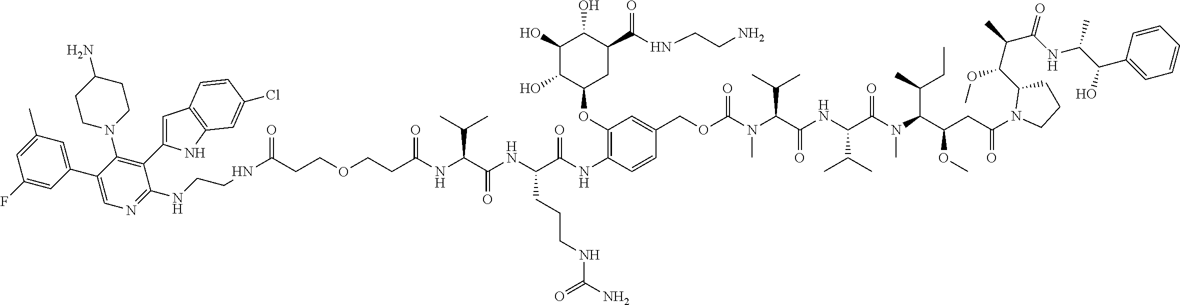 [SMILES: CC[C@H](C)[C@@H]([C@@H](CC(=O)N1CCC[C@H]1[C@H](OC)[C@@H](C)C(=O)N[C@H](C)[C@@H](O)c1ccccc1)OC)N(C)C(=O)[C@@H](NC(=O)[C@H](C(C)C)N(C)C(=O)OCc1ccc(NC(=O)[C@H](CCCNC(N)=O)NC(=O)[C@@H](NC(=O)CCOCCC(=O)NCCNc2ncc(-c3cc(C)cc(F)c3)c(N3CCC(N)CC3)c2-c2cc3ccc(Cl)cc3[nH]2)C(C)C)c(O[C@@H]2C[C@H](C(=O)NCCN)[C@@H](O)[C@H](O)[C@H]2O)c1)C(C)C] | |
| (Compound 43) | |
| [(2S,3S,4S,5R,6S)-6-[5-({[(S)-1-[N-(S)-1-{[(1S,2R)-4-[(S)-2-{(1R,2R)-2-[N-(1R,2S)-2-hydroxy-1-methyl-2- | 1991.0 |
| phenylethylcarbamoyl]-1-methoxypropyl}-1-pyrrolidinyl]-1-[(S)-1-methylpropyl]-2-methoxy-4-oxobutyl]-N- | |
| methylcarbamoyl}-2-methylpropylcarbamoyl]-2-methylpropyl](methyl)(aminocarbonyloxy)}methyl)-2-[(S)-2-[(S)-2-[3-(2-{N- | |
| 2-[4-(4-amino-1-piperidy])-3-(6-chloro-2-indolyl)-5-(3-fluoro-5-tolyl)-2- | |
| pyridylamino]ethylcarbamoyl)ethoxy)propionylamino]-3-methylbutyrylamino]-5-ureidovalerylamino]phenoxy]-3,4,5- | |
| trihydroxytetrahydro-2H-pyran-2-ylcarbonylamino]acetic acid | |
| 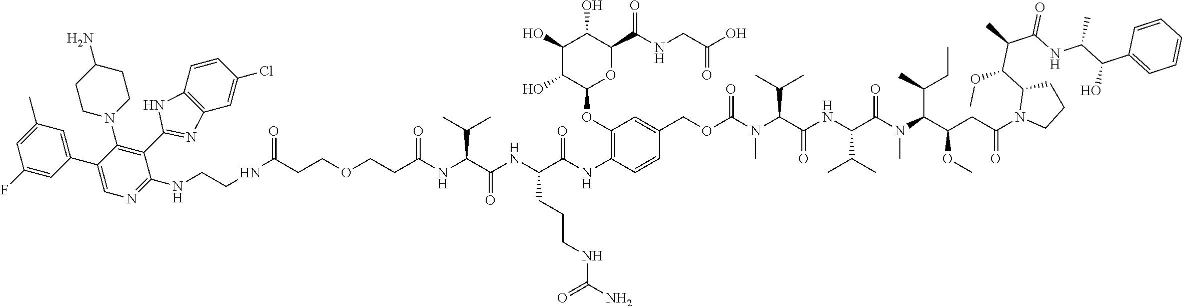 [SMILES: CC[C@H](C)[C@@H]([C@@H](CC(=O)N1CCC[C@H]1[C@H](OC)[C@@H](C)C(=O)N[C@H](C)[C@@H](O)c1ccccc1)OC)N(C)C(=O)[C@@H](NC(=O)[C@H](C(C)C)N(C)C(=O)OCc1ccc(NC(=O)[C@H](CCCNC(N)=O)NC(=O)[C@@H](NC(=O)CCOCCC(=O)NCCNc2ncc(-c3cc(C)cc(F)c3)c(N3CCC(N)CC3)c2-c2nc3cc(Cl)ccc3[nH]2)C(C)C)c(O[C@@H]2O[C@H](C(=O)NCC(=O)O)[C@@H](O)[C@H](O)[C@H]2O)c1)C(C)C] | |
| (Compound 44) | |
| 1-(S)-1-[N-(S)-1-{[(1S,2R)-4-[(S)-2-{(1R,2R)-2-[N-(1R,2S)-2-hydroxy-1-methyl-2-phenylethylcarbamoyl]-1-methoxypropyl}- | 1932.0 |
| 1-pyrrolidinyl]-1-[(S)-1-methylpropyl]-2-methoxy-4-oxobutyl]-N-methylcarbamoyl}-2-methylpropylcarbamoyl]-2- | |
| methylpropyl 1-methyl {3-[(2S,3R,4S,5S,6S)-6-(N-2-aminoethylcarbamoyl)-3,4,5-trihydroxytetrahydro-2H-pyran-2-yloxy]-4- | |
| [(S)-2-[(S)-2-{4-[({N-2-[4-(4-amino-1-piperidyl)-3-(6-chloro-1H-1,3-benzimidazol-2-yl)-5-(3-fluoro-5-tolyl)-2- | |
| pyridylamino]ethylcarbamoyl}methyl)-N-methylcarbamoyl]butyrylamino}-3- | |
| methylbutyrylamino]propionylamino]phenyl}methanecarbamate | |
| 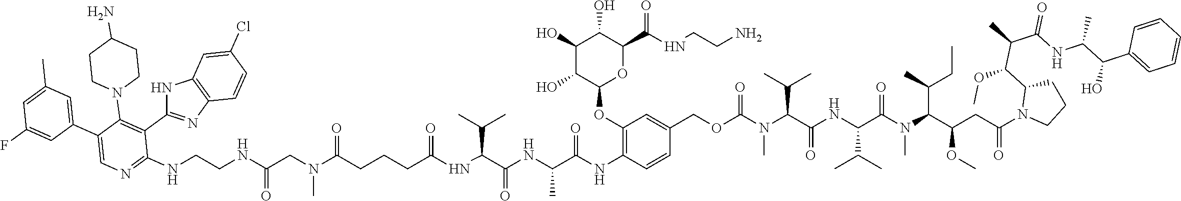 [SMILES: CC[C@H](C)[C@@H]([C@@H](CC(=O)N1CCC[C@H]1[C@H](OC)[C@@H](C)C(=O)N[C@H](C)[C@@H](O)c1ccccc1)OC)N(C)C(=O)[C@@H](NC(=O)[C@H](C(C)C)N(C)C(=O)OCc1ccc(NC(=O)[C@H](C)NC(=O)[C@@H](NC(=O)CCCC(=O)N(C)CC(=O)NCCNc2ncc(-c3cc(C)cc(F)c3)c(N3CCC(N)CC3)c2-c2nc3ccc(Cl)cc3[nH]2)C(C)C)c(O[C@@H]2O[C@H](C(=O)NCCN)[C@@H](O)[C@H](O)[C@H]2O)c1)C(C)C] | |
| (Compound 45) | |
| [(2S,3S,4S,5R,6S)-6-[5-({[(S)-1-[N-(S)-1-{[(1S,2R)-4-[(S)-2-{(1R,2R)-2-[N-(1R,2S)-2-hydroxy-1-methyl-2- | 1904.9 |
| phenylethylcarbamoyl]-1-methoxypropyl}-1-pyrrolidinyl]-1-[(S)-1-methylpropyl]-2-methoxy-4-oxobutyl]-N- | |
| methylcarbamoyl}-2-methylpropylcarbamoyl]-2-methylpropyl](methyl)(aminocarbonyloxy)}methyl)-2-[(S)-2-[(S)-2-{5-[4-(4- | |
| amino-1-piperidyl)-3-(6-chloro-1H-1,3-benzimidazol-2-yl)-5-(3-fluoro-5-tolyl)-2-pyridylamino]valerylamino}-3- | |
| methylbutyrylamino]-5-ureidovalerylamino]phenoxy]-3,4,5-trihydroxytetrahydro-2H-pyran-2-ylcarbonylamino]acetic acid | |
| 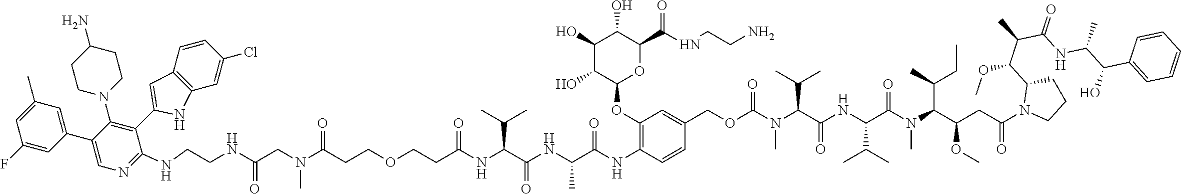 [SMILES: CC[C@H](C)[C@@H]([C@@H](CC(=O)N1CCC[C@H]1[C@H](OC)[C@@H](C)C(=O)N[C@H](C)[C@@H](O)c1ccccc1)OC)N(C)C(=O)[C@@H](NC(=O)[C@H](C(C)C)N(C)C(=O)OCc1ccc(NC(=O)[C@H](C)NC(=O)[C@@H](NC(=O)CCOCCC(=O)N(C)CC(=O)NCCNc2ncc(-c3cc(C)cc(F)c3)c(N3CCC(N)CC3)c2-c2cc3ccc(Cl)cc3[nH]2)C(C)C)c(O[C@@H]2O[C@H](C(=O)NCCN)[C@@H](O)[C@H](O)[C@H]2O)c1)C(C)C] | |
| (Compound 46) | |
| 1-(S)-1-[N-(S)-1-{[(1S,2R)-4-[(S)-2-{(1R,2R)-2-[N-(1R,2S)-2-hydroxy-1-methyl-2-phenylethylcarbamoyl]-1-methoxypropyl}- | 1961.0 |
| 1-pyrrolidinyl]-1-[(S)-1-methylpropyl]-2-methoxy-4-oxobutyl]-N-methylcarbamoyl}-2-methylpropylcarbamoyl]-2- | |
| methylpropyl 1-methyl {3-[(2S,3R,4S,5S,6S)-6-(N-2-aminoethylcarbamoyl)-3,4,5-trihydroxytetrahydro-2H-pyran-2-yloxy]-4- | |
| [(S)-2-[(S)-2-(3-{2-[({N-2-[4-(4-amino-1-piperidyl)-3-(6-chloro-2-indolyl)-5-(3-fluoro-5-tolyl)-2- | |
| pyridylamino]ethylcarbamoyl}methyl)-N-methylcarbamoyl]ethoxy}propionylamino)-3- | |
| methylbutyrylamino]propionylamino]phenyl}methanecarbamate | |
| 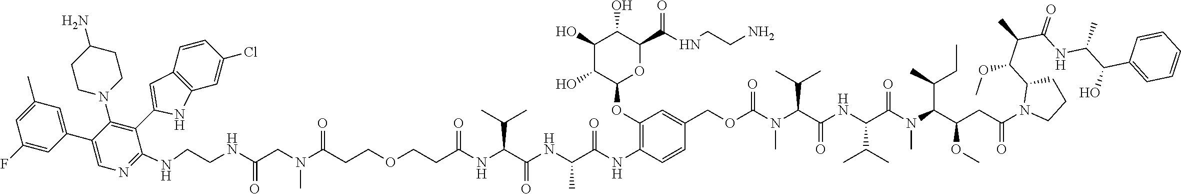 [SMILES: CC[C@H](C)[C@@H]([C@@H](CC(=O)N1CCC[C@H]1[C@H](OC)[C@@H](C)C(=O)N[C@H](C)[C@@H](O)c1ccccc1)OC)N(C)C(=O)[C@@H](NC(=O)[C@H](C(C)C)N(C)C(=O)OCc1ccc(NC(=O)[C@H](C)NC(=O)[C@@H](NC(=O)CCOCCC(=O)N(C)CC(=O)NCCNc2ncc(-c3cc(C)cc(F)c3)c(N3CCC(N)CC3)c2-c2cc3ccc(Cl)cc3[nH]2)C(C)C)c(O[C@@H]2O[C@H](C(=O)NCCN)[C@@H](O)[C@H](O)[C@H]2O)c1)C(C)C] | |
| (Compound 47) | |
| 15-[(28,3S,4S,5R,6S)-3,4,5-trihydroxy-6-[5-({methyl[(S)-2-methyl-1-[N-(S)-1-{[(1S,2R)-2-methoxy-4-[(S)-2-[(1R,2R)-1- | 2174.2 |
| methoxy-2-[N-(1R,2S)-2-hydroxy-1-methyl-2-phenylethylcarbamoyl]propyl]-1-pyrrolidinyl]-1-[(S)-1-methylpropyl]-4- | |
| oxobutyl]-N-methylcarbamoyl}-2-methylpropylcarbamoyl]propyl](aminocarbonyloxy)}methyl)-2-[(S)-2-[(S)-3-methyl-2-[3-(2- | |
| {N-2-[4-(4-amino-1-piperidyl)-3-(6-chloro-1H-1,3-benzimidazol-2-yl)-5-(3-fluoro-5-tolyl)-2- | |
| pyridylamino]ethylcarbamoyl}ethoxy)propionylamino]butyrylamino]-5-ureidovalerylamino]phenoxy]tetrahydro-2H-pyran-2- | |
| ylcarbonylamino]pentadecanoic acid | |
| 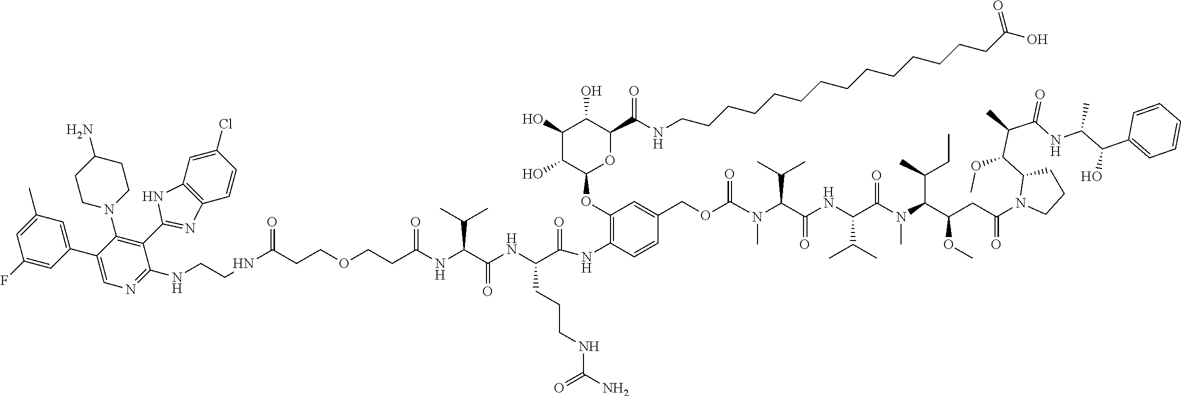 [SMILES: CC[C@H](C)[C@@H]([C@@H](CC(=O)N1CCC[C@H]1[C@H](OC)[C@@H](C)C(=O)N[C@H](C)[C@@H](O)c1ccccc1)OC)N(C)C(=O)[C@@H](NC(=O)[C@H](C(C)C)N(C)C(=O)OCc1ccc(NC(=O)[C@H](CCCNC(N)=O)NC(=O)[C@@H](NC(=O)CCOCCC(=O)NCCNc2ncc(-c3cc(C)cc(F)c3)c(N3CCC(N)CC3)c2-c2nc3ccc(Cl)cc3[nH]2)C(C)C)c(O[C@@H]2O[C@H](C(=O)NCCCCCCCCCCCCCCC(=O)O)[C@@H](O)[C@H](O)[C@H]2O)c1)C(C)C] | |
| (Compound 48) | |
| 3-[(28,3S,4S,5R,6S)-3,4,5-trihydroxy-6-[5-({methyl[(S)-2-methyl-1-[N-(S)-1-{[(1S,2R)-2-methoxy-4-[(S)-2-[(1R,2R)-1- | 2006.0 |
| methoxy-2-[N-(1R,2S)-2-hydroxy-1-methyl-2-phenylethylcarbamoyl]propyl]-1-pyrrolidinyl]-1-[(S)-1-methylpropyl]-4- | |
| oxobutyl]-N-methylcarbamoyl}-2-methylpropylcarbamoyl]propyl](aminocarbonyloxy)}methyl)-2-[(S)-2-[(S)-3-methyl-2-[3-(2- | |
| {N-2-[4-(4-amino-1-piperidyl)-3-(6-chloro-1H-1,3-benzimidazol-2-yl)-5-(3-fluoro-5-tolyl)-2- | |
| pyridylamino]ethylcarbamoyl}ethoxy)propionylamino]butyrylamino]-5-ureidovalerylamino]phenoxy]tetrahydro-2H-pyran-2- | |
| ylcarbonylamino]propionic acid | |
| 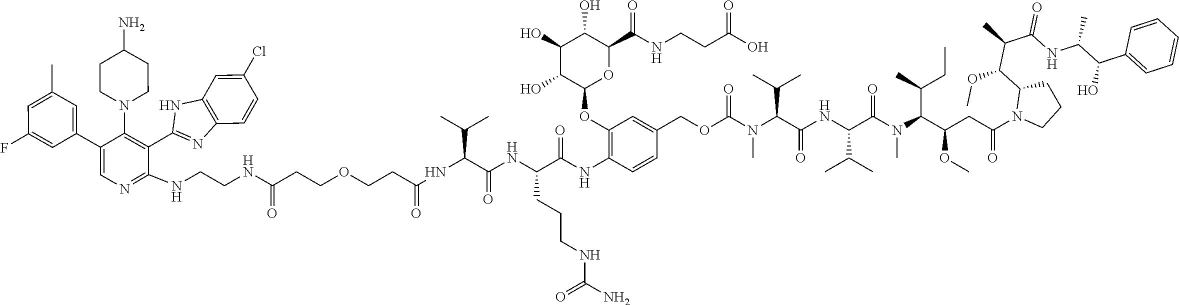 [SMILES: CC[C@H](C)[C@@H]([C@@H](CC(=O)N1CCC[C@H]1[C@H](OC)[C@@H](C)C(=O)N[C@H](C)[C@@H](O)c1ccccc1)OC)N(C)C(=O)[C@@H](NC(=O)[C@H](C(C)C)N(C)C(=O)OCc1ccc(NC(=O)[C@H](CCCNC(N)=O)NC(=O)[C@@H](NC(=O)CCOCCC(=O)NCCNc2ncc(-c3cc(C)cc(F)c3)c(N3CCC(N)CC3)c2-c2nc3ccc(Cl)cc3[nH]2)C(C)C)c(O[C@@H]2O[C@H](C(=O)NCCC(=O)O)[C@@H](O)[C@H](O)[C@H]2O)c1)C(C)C] | |
| (Compound 49) | |
| [(2S,3S,4S,5R,6S)-3,4,5-trihydroxy-6-[5-({methyl[(S)-2-methyl-1-[N-(S)-1-{[(1S,2R)-2-methoxy-4-[(S)-2-[(1R,2R)-1- | 2012.9 |
| methoxy-2-[N-(1R,2S)-2-hydroxy-1-methyl-2-phenylethylcarbamoyl]propyl]-1-pyrrolidinyl]-1-[(S)-1-methylpropyl]-4- | |
| oxobutyl]-N-methylcarbamoyl}-2-methylpropylcarbamoyl]propyl] | |
| (aminocarbonyloxy)}methyl)-2-[(S)-2-[(S)-3-methyl-2-(3-{2-[N-methyl({N-2-[4-(4-amino-1-piperidy])-3-(6-chloro-1H-1,3- | |
| benzimidazol-2-yl)-5-(3-fluoro-5-tolyl)-2-pyridylamino] | |
| ethylcarbamoyl}methyl)carbamoyl]ethoxy}propionylamino) | |
| butyrylamino]propionylamino]phenoxy]tetrahydro-2H-pyran-2-ylcarbonylamino]methanesulfonic acid | |
| 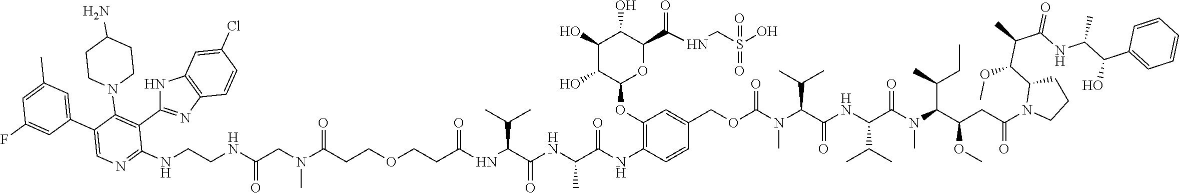 [SMILES: CC[C@H](C)[C@@H]([C@@H](CC(=O)N1CCC[C@H]1[C@H](OC)[C@@H](C)C(=O)N[C@H](C)[C@@H](O)c1ccccc1)OC)N(C)C(=O)[C@@H](NC(=O)[C@H](C(C)C)N(C)C(=O)OCc1ccc(NC(=O)[C@H](C)NC(=O)[C@@H](NC(=O)CCOCCC(=O)N(C)CC(=O)NCCNc2ncc(-c3cc(C)cc(F)c3)c(N3CCC(N)CC3)c2-c2nc3ccc(Cl)cc3[nH]2)C(C)C)c(O[C@@H]2O[C@H](C(=O)NCS(=O)(=O)O)[C@@H](O)[C@H](O)[C@H]2O)c1)C(C)C] | |
| (Compound 50) | |
| 2-[(2S,3S,4S,5R,6S)-3,4,5-trihydroxy-6-[5-({methyl[(S)-2-methyl-1-[N-(S)-1-{[(1S,2R)-2-methoxy-4-[(S)-2-[(1R,2R)-1- | 2026.9 |
| methoxy-2-[N-(1R,2S)-2-hydroxy-1-methyl-2-phenylethylcarbamoyl]propyl]-1-pyrrolidinyl]-1-[(S)-1-methylpropyl]-4- | |
| oxobutyl]-N-methylcarbamoyl}-2-methylpropylcarbamoyl]propyl](aminocarbonyloxy)}methyl)-2-[(S)-2-[(S)-3-methyl-2-(3- | |
| {2-[N-methyl({N-2-[4-(4-amino-1-piperidyl)-3-(6-chloro-1H-1,3-benzimidazol-2-y])-5-(3-fluoro-S-tolyl)-2- | |
| pyridylamino]ethylcarbamoyl}methyl)carbamoyl]ethoxy}propionylamino) | |
| butyrylamino]propionylamino]phenoxy]tetrahydro-2H-pyran-2-ylcarbonylamino]ethanesulfonic acid | |
| 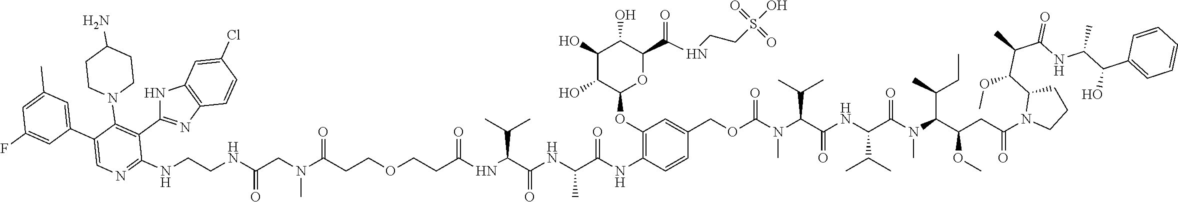 [SMILES: CC[C@H](C)[C@@H]([C@@H](CC(=O)N1CCC[C@H]1[C@H](OC)[C@@H](C)C(=O)N[C@H](C)[C@@H](O)c1ccccc1)OC)N(C)C(=O)[C@@H](NC(=O)[C@H](C(C)C)N(C)C(=O)OCc1ccc(NC(=O)[C@H](C)NC(=O)[C@@H](NC(=O)CCOCCC(=O)N(C)CC(=O)NCCNc2ncc(-c3cc(C)cc(F)c3)c(N3CCC(N)CC3)c2-c2nc3ccc(Cl)cc3[nH]2)C(C)C)c(O[C@@H]2O[C@H](C(=O)NCCS(=O)(=O)O)[C@@H](O)[C@H](O)[C@H]2O)c1)C(C)C] | |
| (Compound 51) | |
| 15-[N-2-[(2S,3S,4S,5R,6S)-3,4,5-trihydroxy-6-[5-({methyl[(S)-2-methyl-1-[N-(S)-1-{[(1S,2R)-2-methoxy-4-[(S)-2-[(1R,2R)- | 2253.1 |
| 1-methoxy-2-[N-(1R,2S)-2-hydroxy-1-methyl-2-phenylethylcarbamoyl]propyl]-1-pyrrolidinyl]-1-[(S)-1-methylpropyl]-4- | |
| oxobutyl]-N-methylcarbamoyl}-2-methylpropylcarbamoyl]propyl](aminocarbonyloxy)}methyl)-2-[(S)-2-[(S)-3-methyl-2-[3-(2- | |
| {N-2-[4-(4-amino-1-piperidyl)-3-(6-chloro-1H-1,3-benzimidazol-2-yl)-5-(3-fluoro-5-tolyl)-2- | |
| pyridylamino]ethylcarbamoyl}ethoxy)propionylamino]butyrylamino]-5-ureidovalerylamino]phenoxy]tetrahydro-2H-pyran-2- | |
| ylcarbonylamino]ethylcarbamoyl}-4,7,10,13-tetraoxapentadecanoic acid | |
| 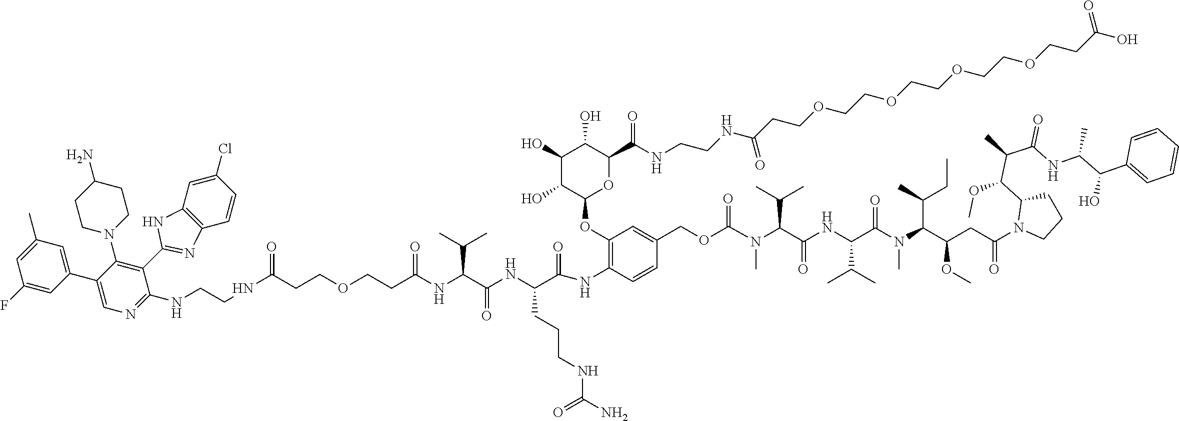 [SMILES: CC[C@H](C)[C@@H]([C@@H](CC(=O)N1CCC[C@H]1[C@H](OC)[C@@H](C)C(=O)N[C@H](C)[C@@H](O)c1ccccc1)OC)N(C)C(=O)[C@@H](NC(=O)[C@H](C(C)C)N(C)C(=O)OCc1ccc(NC(=O)[C@H](CCCNC(N)=O)NC(=O)[C@@H](NC(=O)CCOCCC(=O)NCCNc2ncc(-c3cc(C)cc(F)c3)c(N3CCC(N)CC3)c2-c2nc3ccc(Cl)cc3[nH]2)C(C)C)c(O[C@@H]2O[C@H](C(=O)NCCNC(=O)CCOCCOCCOCCOCCC(=O)O)[C@@H](O)[C@H](O)[C@H]2O)c1)C(C)C] | |
| (Compound 52) | |
| 15-(N-2-[(28,3S,4S,5R,6S)-3,4,5-trihydroxy-6-[5-({methyl[(S)-2-methyl-1-[N-(S)-1-{[(1S,2R)-2-methoxy-4-[(S)-2-[(1R,2R)- | 2245.2 |
| 1-methoxy-2-[N-(1R,2S)-2-hydroxy-1-methyl-2-phenylethylcarbamoyl]propyl]-1-pyrrolidinyl]-1-[(S)-1-methylpropyl]-4- | |
| oxobutyl]-N-methylcarbamoyl}-2-methylpropylcarbamoyl]propyl](aminocarbonyloxy)}methyl)-2-[(S)-2-[(S)-3-methyl-2-[3-(2- | |
| {N-2-[4-(4-amino-1-piperidyl)-3-(6-chloro-1H-1,3-benzimidazol-2-yl)-5-(3-fluoro-5-tolyl)-2- | |
| pyridylamino]ethylcarbamoyl}ethoxy)propionylamino]butyrylamino]-5-ureidovalerylamino]phenoxy]tetrahydro-2H-pyran-2- | |
| ylcarbonylamino]ethylcarbamoyl}pentadecanoic acid | |
| 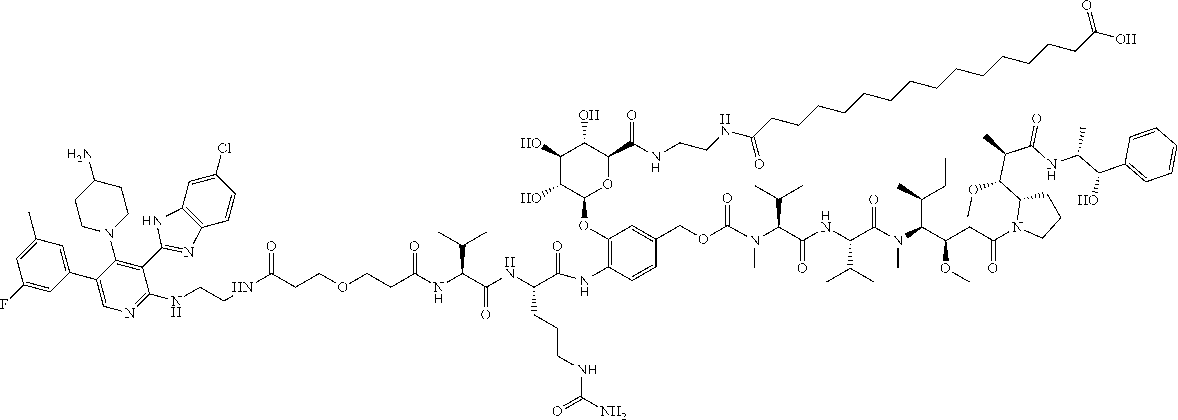 [SMILES: CC[C@H](C)[C@@H]([C@@H](CC(=O)N1CCC[C@H]1[C@H](OC)[C@@H](C)C(=O)N[C@H](C)[C@@H](O)c1ccccc1)OC)N(C)C(=O)[C@@H](NC(=O)[C@H](C(C)C)N(C)C(=O)OCc1ccc(NC(=O)[C@H](CCCNC(N)=O)NC(=O)[C@@H](NC(=O)CCOCCC(=O)NCCNc2ncc(-c3cc(C)cc(F)c3)c(N3CCC(N)CC3)c2-c2nc3ccc(Cl)cc3[nH]2)C(C)C)c(O[C@@H]2O[C@H](C(=O)NCCNC(=O)CCCCCCCCCCCCCCC(=O)O)[C@@H](O)[C@H](O)[C@H]2O)c1)C(C)C] | |
| (Compound 53) | |
| (S)-2,6-bis[(2S,3S,4S,5R,6S)-3,4,5-trihydroxy-6-[5-({methyl[(S)-2-methyl-1-[N-(S)-1-{[(1S,2R)-2-methoxy-4-[(S)-2-[(1R,2R)- | 2349.0 |
| 1-methoxy-2-[N-(1R,2S)-2-hydroxy-1-methyl-2-phenylethylcarbamoyl]propyl]-1-pyrrolidinyl]-1-[(S)-1-methylpropyl]-4- | |
| oxobutyl]-N-methylcarbamoyl}-2-methylpropylcarbamoyl]propyl](aminocarbonyloxy)}methyl)-2-[(S)-2-[(S)-3-methyl-2-[3-(2- | |
| {N-2-[4-(4-amino-1-piperidyl)-3-(6-chloro-1H-1,3-benzimidazol-2-yl)-5-(3-fluoro-5-tolyl)-2- | |
| pyridylamino]ethylcarbamoyl}ethoxy)propionylamino]butyrylamino]-5-ureidovalerylamino]phenoxy]tetrahydro-2H-pyran-2- | |
| ylcarbonylaminolhexanoic acid | |
| 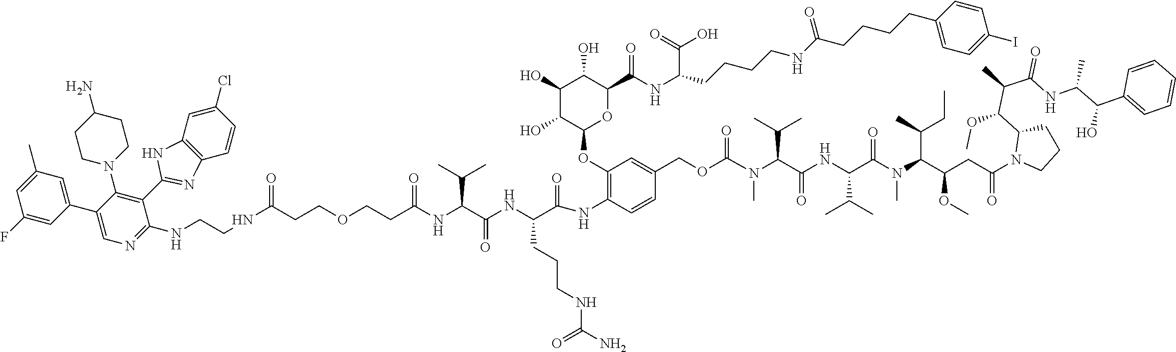 [SMILES: CC[C@H](C)[C@@H]([C@@H](CC(=O)N1CCC[C@H]1[C@H](OC)[C@@H](C)C(=O)N[C@H](C)[C@@H](O)c1ccccc1)OC)N(C)C(=O)[C@@H](NC(=O)[C@H](C(C)C)N(C)C(=O)OCc1ccc(NC(=O)[C@H](CCCNC(N)=O)NC(=O)[C@@H](NC(=O)CCOCCC(=O)NCCNc2ncc(-c3cc(C)cc(F)c3)c(N3CCC(N)CC3)c2-c2nc3ccc(Cl)cc3[nH]2)C(C)C)c(O[C@@H]2O[C@H](C(=O)N[C@@H](CCCCNC(=O)CCCCc3ccc(I)cc3)C(=O)O)[C@@H](O)[C@H](O)[C@H]2O)c1)C(C)C] | |
| (Compound 54) | |
| (S)-2,6-bis {3-[(2S,3S,4S,5R,6S)-3,4,5-trihydroxy-6-[5-({methyl[(S)-2-methyl-1-[N-(S)-1-{[(1S,2R)-2-methoxy-4-[(S)-2- | 2420.1 |
| [(1R,2R)-1-methoxy-2-[N-(1R.2S)-2-hydroxy-1-methyl-2-phenylethylcarbamoyl]propyl]-1-pyrrolidinyl]-1-[(S)-1- | |
| methylpropyl]-4-oxobutyl]-N-methylcarbamoyl}-2-methylpropylcarbamoyl]propyl](aminocarbonyloxy)}methyl)-2-[(S)-2-[(S)- | |
| 3-methyl-2-[3-(2-{N-2-[4-(4-amino-1-piperidyl)-3-(6-chloro-1H-1,3-benzimidazol-2-yl)-5-(3-fluoro-5-tolyl)-2- | |
| pyridylamino]ethylcarbamoyl}ethoxy)propionylamino]butyrylamino]-5-ureidovalerylamino]phenoxy]tetrahydro-2H-pyran-2- | |
| ylcarbonylamino]propionylamino}hexanoic acid | |
| 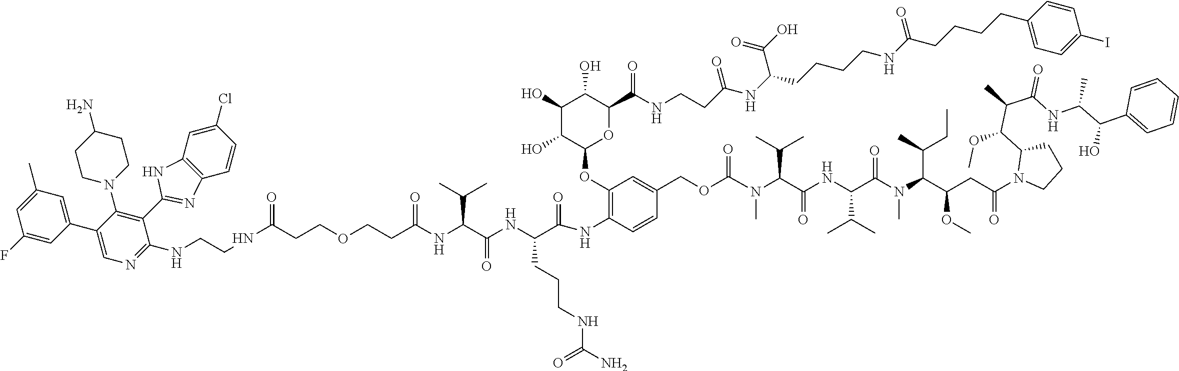 [SMILES: CC[C@H](C)[C@@H]([C@@H](CC(=O)N1CCC[C@H]1[C@H](OC)[C@@H](C)C(=O)N[C@H](C)[C@@H](O)c1ccccc1)OC)N(C)C(=O)[C@@H](NC(=O)[C@H](C(C)C)N(C)C(=O)OCc1ccc(NC(=O)[C@H](CCCNC(N)=O)NC(=O)[C@@H](NC(=O)CCOCCC(=O)NCCNc2ncc(-c3cc(C)cc(F)c3)c(N3CCC(N)CC3)c2-c2nc3ccc(Cl)cc3[nH]2)C(C)C)c(O[C@@H]2O[C@H](C(=O)NCCC(=O)N[C@@H](CCCCNC(=O)CCCCc3ccc(I)cc3)C(=O)O)[C@@H](O)[C@H](O)[C@H]2O)c1)C(C)C] | |
| (Compound 55) | |
| 1-(S)-1-[N-(S)-1-{[(1S,2R)-4-[(S)-2-{(1R,2R)-2-[N-(1R,2S)-2-hydroxy-1-methyl-2-phenylethylcarbamoyl]-1-methoxypropyl}- | 2048.0 |
| 1-pyrrolidinyl]-1-[(S)-1-methylpropyl]-2-methoxy-4-oxobutyl]-N-methylcarbamoyl}-2-methylpropylcarbamoyl]-2- | |
| methylpropyl 1-methyl {3-[(2S,3R,4S,5S,6S)-6-(N-2-aminoethylcarbamoyl)-3,4,5-trihydroxytetrahydro-2H-pyran-2-yloxy]-4- | |
| [(S)-2-[(S)-2-(3-{2-[{{N-2-[4-(4-amino-1-piperidy])-3-(6-chloro-1H-1,3-benzimidazol-2-yl)-5-(3-fluoro-5-tolyl)-2- | |
| pyridylamino]ethylcarbamoyl}methyl)-N-methylcarbamoyl]ethoxy}propionylamino)-3-methylbutyrylamino]-5- | |
| ureidovalerylamino]phenyl}methanecarbamate | |
| 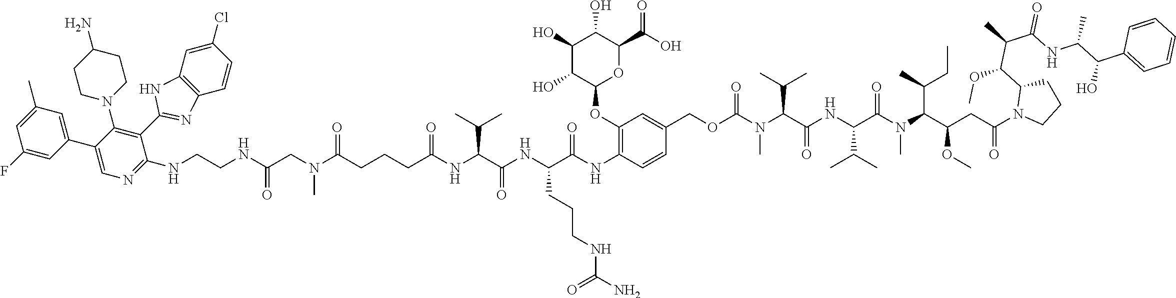 [SMILES: CC[C@H](C)[C@@H]([C@@H](CC(=O)N1CCC[C@H]1[C@H](OC)[C@@H](C)C(=O)N[C@H](C)[C@@H](O)c1ccccc1)OC)N(C)C(=O)[C@@H](NC(=O)[C@H](C(C)C)N(C)C(=O)OCc1ccc(NC(=O)[C@H](CCCNC(N)=O)NC(=O)[C@@H](NC(=O)CCCC(=O)N(C)CC(=O)NCCNc2ncc(-c3cc(C)cc(F)c3)c(N3CCC(N)CC3)c2-c2nc3ccc(Cl)cc3[nH]2)C(C)C)c(O[C@@H]2O[C@H](C(=O)O)[C@@H](O)[C@H](O)[C@H]2O)c1)C(C)C] | |
| (Compound 56) | |
| [(2S,3S,4S,5R,6S)-6-{2-{(S)-2-[(S)-2-{8-[4-(4-amino-1-piperidyl)-3-(6-chloro-1H-1,3-benzimidazol-2-yl)-5-(3-fluoro-5-tolyl)- | 1961.0 |
| 2-pyridylamino]octylcarbonylamino}-3-methylbutyrylamino]-5-ureidovalerylamino]-5-({methyl[(S)-2-methyl-1-[N-(S)-1- | |
| {[(1S,2R)-2-methoxy-4-[(S)-2-[(1R,2R)-1-methoxy-2-[N-(1R,2S)-2-hydroxy-1-methyl-2-phenylethylcarbamoyl]propyl]-1- | |
| pyrrolidinyl]-1-[(S)-1-methylpropyl]-4-oxobuty]]-N-methylcarbamoyl}-2- | |
| methylpropylcarbamoyl]propyl](aminocarbonyloxy)}methyl)phenoxy}-3,4,5-trihydroxytetrahydro-2H-pyran-2- | |
| ylcarbonylamino]acetic acid | |
| 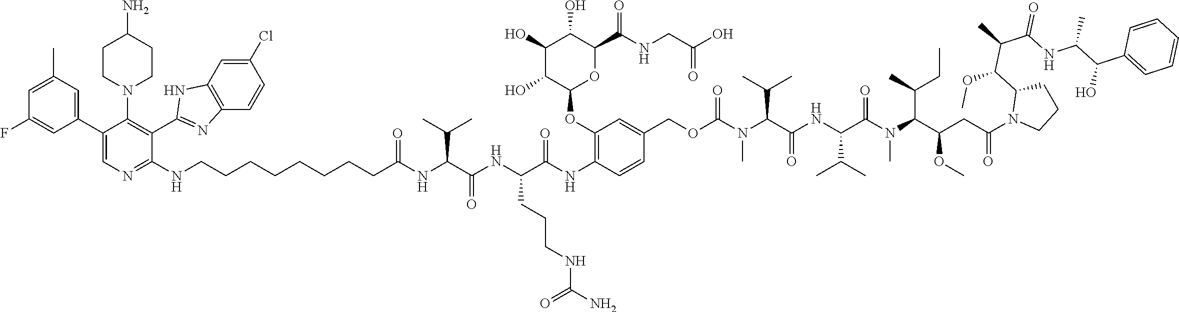 [SMILES: CC[C@H](C)[C@@H]([C@@H](CC(=O)N1CCC[C@H]1[C@H](OC)[C@@H](C)C(=O)N[C@H](C)[C@@H](O)c1ccccc1)OC)N(C)C(=O)[C@@H](NC(=O)[C@H](C(C)C)N(C)C(=O)OCc1ccc(NC(=O)[C@H](CCCNC(N)=O)NC(=O)[C@@H](NC(=O)CCCCCCCCNc2ncc(-c3cc(C)cc(F)c3)c(N3CCC(N)CC3)c2-c2nc3ccc(Cl)cc3[nH]2)C(C)C)c(O[C@@H]2O[C@H](C(=O)NCC(=O)O)[C@@H](O)[C@H](O)[C@H]2O)c1)C(C)C] | |
| (Compound 193) | |
| 1-methyl 1-(S)-2-methyl-1-[N-(S)-1-{[(1S,2R)-2-methoxy-4-[(S)-2-[(1R,2R)-1-methoxy-2-[N-(1R,2S)-2-hydroxy-1-methyl-2- | 1963.0 |
| phenylethylcarbamoyl]propyl]-1-pyrrolidinyl]-1-[(S)-1-methylpropyl]-4-oxobutyl]-N-methylcarbamoyl}-2- | |
| methylpropylcarbamoyl]propyl {4-[(S)-2-[(S)-3-methyl-2-(3-{2-[N-methyl({N-2-[4-(4-amino-1-piperidyl)-3-(6-chloro-1H-1,3- | |
| benzimidazol-2-yl)-5-(3-fluoro-5-toly])-2- | |
| pyridylamino]etbylcarbamoyl}methyl)carbamoyl]ethoxy}propionylamino)butyrylamino]propionylamino]-3-[(2S,3R,4S,5S,6S)- | |
| 3,4,5-trihydroxy-6-(N-2-hydroxyethylcarbamoyl)tetrahydro-2H-pyran-2-yloxy]phenyl}methanecarbamate | |
| 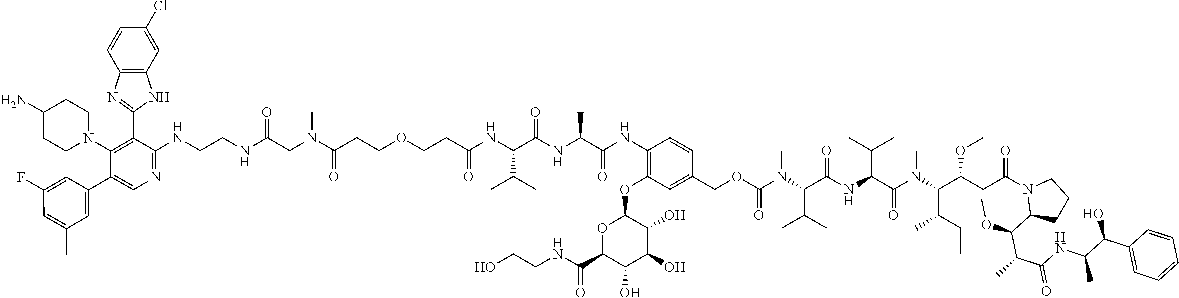 [SMILES: CC[C@H](C)[C@@H]([C@@H](CC(=O)N1CCC[C@H]1[C@H](OC)[C@@H](C)C(=O)N[C@H](C)[C@@H](O)c1ccccc1)OC)N(C)C(=O)[C@@H](NC(=O)[C@H](C(C)C)N(C)C(=O)OCc1ccc(NC(=O)[C@H](C)NC(=O)[C@@H](NC(=O)CCOCCC(=O)N(C)CC(=O)NCCNc2ncc(-c3cc(C)cc(F)c3)c(N3CCC(N)CC3)c2-c2nc3ccc(Cl)cc3[nH]2)C(C)C)c(O[C@@H]2O[C@H](C(=O)NCCO)[C@@H](O)[C@H](O)[C@H]2O)c1)C(C)C] | |
| (Compound 206) | |
| 1-methyl 1-(S)-2-methyl-1-[N-(S)-1-{[(1S,2R)-2-methoxy-4-[(S)-2-[(1R,2R)-1-methoxy-2-[N-(1R,2S)-2-hydroxy-1-methyl-2- | 2049.0 |
| phenylethylcarbamoyl]propyl]-1-pyrrolidinyl]-1-[(S)-1-methylpropyl]-4-oxobutyl]-N-methylcarbamoyl}-2- | |
| methylpropylcarbamoyl]propyl{4-[(S)-2-[(S)-3-methyl-2-(3-{2-[N-methyl({N-2-[4-(4-amino-1-piperidyl)-3-(6-chloro-1H-1,3- | |
| benzimidazol-2-yl)-5-(3-fluoro-5-toly])-2- | |
| pyridylamino]ethylcarbamoyl}methyl)carbamoyl]ethoxy}propionylamino)butyrylamino]-5-ureidovalerylamino]-3- | |
| [(2S,3R,4S,5S,6S)-3,4,5-trihydroxy-6-(N-2-hydroxyethylcarbamoyl)tetrahydro-2H-pyran-2-yloxy]phenyl}methanecarbamate | |
| 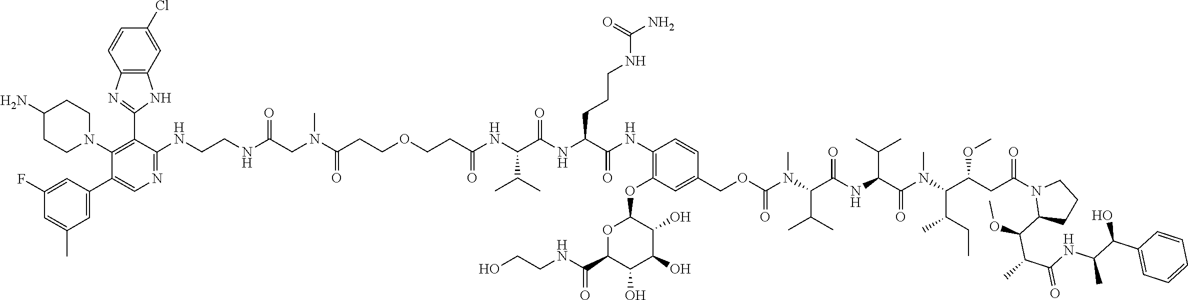 [SMILES: CC[C@H](C)[C@@H]([C@@H](CC(=O)N1CCC[C@H]1[C@H](OC)[C@@H](C)C(=O)N[C@H](C)[C@@H](O)c1ccccc1)OC)N(C)C(=O)[C@@H](NC(=O)[C@H](C(C)C)N(C)C(=O)OCc1ccc(NC(=O)[C@H](CCCNC(N)=O)NC(=O)[C@@H](NC(=O)CCOCCC(=O)N(C)CC(=O)NCCNc2ncc(-c3cc(C)cc(F)c3)c(N3CCC(N)CC3)c2-c2nc3ccc(Cl)cc3[nH]2)C(C)C)c(O[C@@H]2O[C@H](C(=O)NCCO)[C@@H](O)[C@H](O)[C@H]2O)c1)C(C)C] | |
| (Compound 207) | |
| 2-[(2S,3S,4S,5R,6S)-3,4,5-trihydroxy-6-[5-({methyl[(S)-2-methyl-1-[N-(S)-1-{[(1S,2R)-2-methoxy-4-[(S)-2-[(1R,2R)-1- | 2041.9 |
| methoxy-2-[N-(1R,2S)-2-hydroxy-1-methyl-2-phenylethylcarbamoyl]propyl]-1-pyrrolidinyl]-1-[(S)-1-methylpropyl]-4- | |
| oxobutyl]-N-methylcarbamoyl}-2-methylpropylcarbamoyl]propyl](aminocarbonyloxy)}methyl)-2-[(S)-2-[(S)-3-methyl-2-[3-(2- | |
| {N-2-[4-(4-amino-1-piperidyl)-3-(6-chloro-1H-1,3-benzimidazol-2-yl)-5-(3-fluoro-5-tolyl)-2- | |
| pyridylamino]ethylcarbamoyl}ethoxy)propionylamino]butyrylamino]-5-ureidovalerylamino]phenoxy]tetrahydro-2H-pyran-2- | |
| ylcarbonylamino]ethanesulfonic acid | |
| 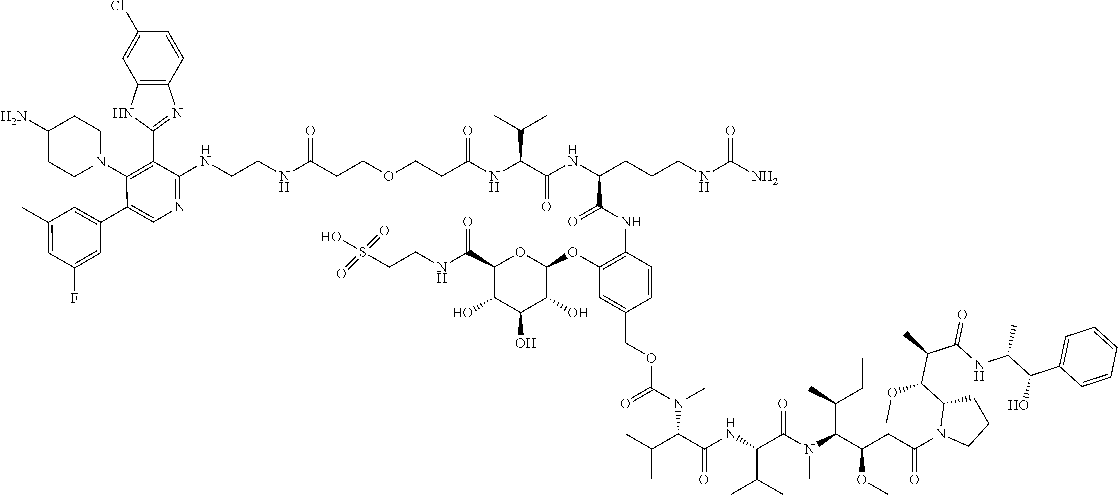 [SMILES: CC[C@H](C)[C@@H]([C@@H](CC(=O)N1CCC[C@H]1[C@H](OC)[C@@H](C)C(=O)N[C@H](C)[C@@H](O)c1ccccc1)OC)N(C)C(=O)[C@@H](NC(=O)[C@H](C(C)C)N(C)C(=O)OCc1ccc(NC(=O)[C@H](CCCNC(N)=O)NC(=O)[C@@H](NC(=O)CCOCCC(=O)NCCNc2ncc(-c3cc(C)cc(F)c3)c(N3CCC(N)CC3)c2-c2nc3ccc(Cl)cc3[nH]2)C(C)C)c(O[C@@H]2O[C@H](C(=O)NCCS(=O)(=O)O)[C@@H](O)[C@H](O)[C@H]2O)c1)C(C)C] | |
| (Compound 208) | |
| 1-methyl 1-(S)-2-methyl-1-[N-(S)-1-{[(1S,2R)-2-methoxy-4-[(S)-2-[(1R,2R)-1-methoxy-2-[N-(1R,2S)-2-hydroxy-1-methyl-2- | 2018.0 |
| phenylethylcarbamoyl]propyl]-1-pyrrolidinyl]-1-[(S)-1-methylpropyl]-4-oxobutyl]-N-methylcarbamoyl}-2- | |
| methylpropylcarbamoyl]propyl{4-[(S)-2-[(S)-3-methyl-2-{4-[N-methyl({N-2-[4-(4-amino-1-piperidyl)-3-(6-chloro-1H-1,3- | |
| benzimidazol-2-yl)-5-(3-fluoro-5-tolyl)-2-pyridylamino]ethylcarbamoyl}methyl)carbamoyl]butyrylamino}butyrylamino]-5- | |
| ureidovalerylamino]-3-[(2S,3R,4S,5S,6S)-3,4,5-trihydroxy-6-(N-2-aminoethylcarbamoyl)tetrahydro-2H-pyran-2- | |
| yloxy]phenyl}methanecarbamate | |
| 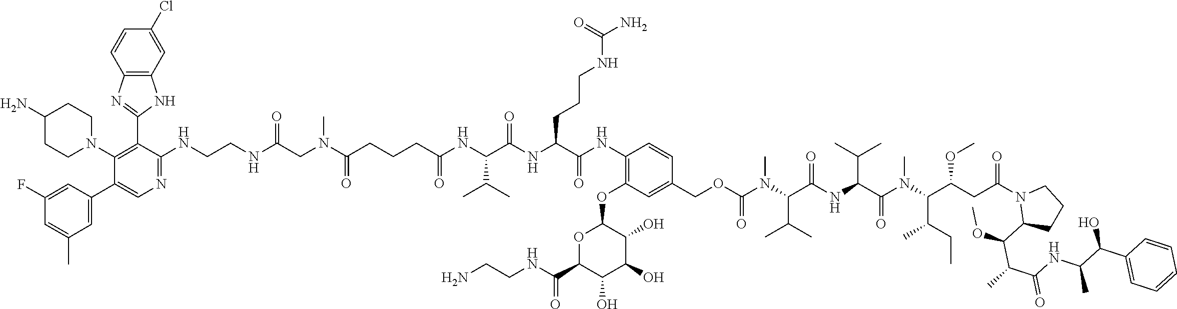 [SMILES: CC[C@H](C)[C@@H]([C@@H](CC(=O)N1CCC[C@H]1[C@H](OC)[C@@H](C)C(=O)N[C@H](C)[C@@H](O)c1ccccc1)OC)N(C)C(=O)[C@@H](NC(=O)[C@H](C(C)C)N(C)C(=O)OCc1ccc(NC(=O)[C@H](CCCNC(N)=O)NC(=O)[C@@H](NC(=O)CCCC(=O)N(C)CC(=O)NCCNc2ncc(-c3cc(C)cc(F)c3)c(N3CCC(N)CC3)c2-c2nc3ccc(Cl)cc3[nH]2)C(C)C)c(O[C@@H]2O[C@H](C(=O)NCCN)[C@@H](O)[C@H](O)[C@H]2O)c1)C(C)C] | |
| (Compound 209) | |
| [(28,3S,4S,5R,6S)-3,4,5-trihydroxy-6-[5-({methyl[(S)-2-methyl-1-[N-(S)-1-{[(1S,2R)-2-methoxy-4-[(S)-2-[(1R,2R)-1- | 1989.9 |
| methoxy-2-[N-(1R,2S)-2-hydroxy-1-methyl-2-phenylethylcarbamoyl]propyl]-1-pyrrolidinyl]-1-[(S)-1-methylpropyl]-4- | |
| oxobutyl]-N-methylcarbamoyl}-2-methylpropylcarbamoyl]propyl](aminocarbonyloxy)}methyl)-2-[(S)-2-{1-[3-(2-{N-2-[4-(4- | |
| amino-1-piperidyl)-3-(6-chloro-1H-1,3-benzimidazol-2-yl)-5-(3-fluoro-5-tolyl)-2- | |
| pyridylamino]ethylcarbamoyl}ethoxy)propionylamino]cyclovalerylamino}-5-ureidovalerylamino]phenoxy]tetrahydro-2H- | |
| pyran-2-ylcarbonylamino]acetic acid | |
| 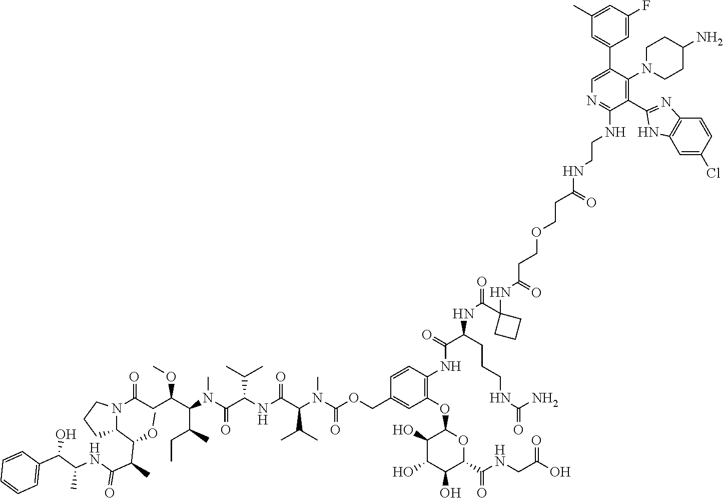 [SMILES: CC[C@H](C)[C@@H]([C@@H](CC(=O)N1CCC[C@H]1[C@H](OC)[C@@H](C)C(=O)N[C@H](C)[C@@H](O)c1ccccc1)OC)N(C)C(=O)[C@@H](NC(=O)[C@H](C(C)C)N(C)C(=O)OCc1ccc(NC(=O)[C@H](CCCNC(N)=O)NC(=O)C2(NC(=O)CCOCCC(=O)NCCNc3ncc(-c4cc(C)cc(F)c4)c(N4CCC(N)CC4)c3-c3nc4ccc(Cl)cc4[nH]3)CCC2)c(O[C@H]2O[C@H](C(=O)NCC(=O)O)[C@@H](O)[C@H](O)[C@H]2O)c1)C(C)C] | |
| (Compound 210) | |
| (S)-2,6-bis[(2S,3S,4S,5R,6S)-6-{2-[(S)-2-[(S)-2-[(R)-3-amino-3-[N-2-(2-{N-2-[4-(4-amino-1-piperidyl)-3-(6-chloro-1H-1,3- | 2435.1 |
| benzimidazol-2-yl)-5-(3-fluoro-5-tolyl)-2-pyridylamino]ethylcarbamoyl}ethoxy)ethylcarbamoyl]propionylamino]-3- | |
| methylbutyrylamino]-5-ureidovalerylamino]-5-({methyl[(S)-2-methyl-1-[N-(S)-1-{[(1S,2R)-2-methoxy-4-[(S)-2-[(1R,2R)-1- | |
| methoxy-2-[N-(1R,2S)-2-hydroxy-1-methyl-2-phenylethylcarbamoyl]propyl]-1-pyrrolidinyl]-1-[(S)-1-methylpropyl]-4- | |
| oxobutyl]-N-methylcarbamoyl}-2-methylpropylcarbamoyl]propyl](aminocarbonyloxy)}methyl)phenoxy}-3,4,5- | |
| trihydroxytetrahydro-2H-pyran-2-ylcarbonylamino]hexanoic acid | |
| 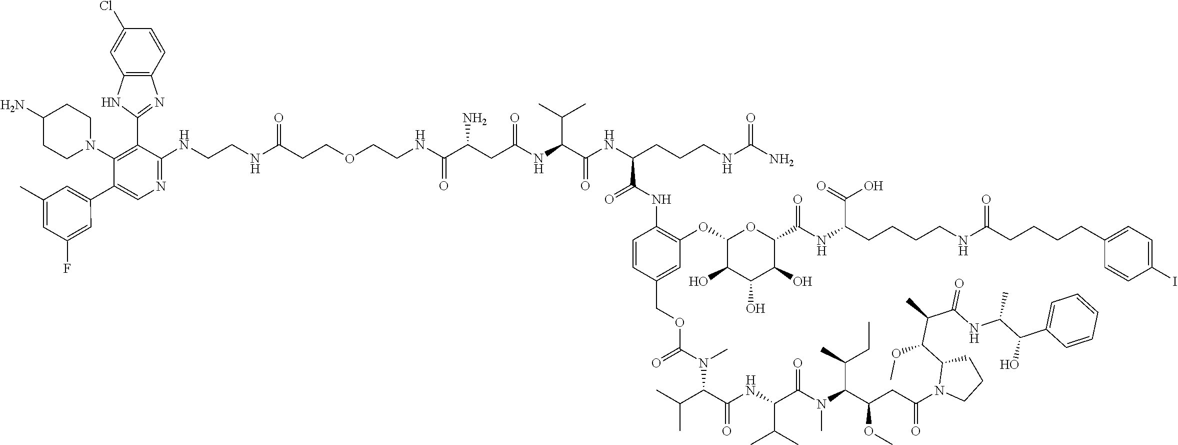 [SMILES: CC[C@H](C)[C@@H]([C@@H](CC(=O)N1CCC[C@H]1[C@H](OC)[C@@H](C)C(=O)N[C@H](C)[C@@H](O)c1ccccc1)OC)N(C)C(=O)[C@@H](NC(=O)[C@H](C(C)C)N(C)C(=O)OCc1ccc(NC(=O)[C@H](CCCNC(N)=O)NC(=O)[C@@H](NC(=O)C[C@@H](N)C(=O)NCCOCCC(=O)NCCNc2ncc(-c3cc(C)cc(F)c3)c(N3CCC(N)CC3)c2-c2nc3ccc(Cl)cc3[nH]2)C(C)C)c(O[C@@H]2O[C@H](C(=O)N[C@@H](CCCCNC(=O)CCCCc3ccc(I)cc3)C(=O)O)[C@@H](O)[C@H](O)[C@H]2O)c1)C(C)C] | |
| (Compound 211) | |
| [(2S,3S,4S,5R,6S)-3,4,5-trihydroxy-6-{2-[(S)-2-[(S)-2-[(S)-3-(4-imidazolyl)-2-[3-(2-{N-2-[4-(4-amino-1-piperidyl)-3-(6- | 2129.0 |
| chloro-1H-1,3-benzimidazol-2-yl)-5-(3-fluoro-5-tolyl)-2- | |
| pyridylamino]ethylcarbamoyl}ethoxy)propionylamino]propionylamino]-3-methylbutyrylamino]-5-ureidovalerylamino]-5- | |
| ({methyl[(S)-2-methyl-1-[N-(S)-1-{[(1S,2R)-2-methoxy-4-[(S)-2-[(1R,2R)-1-methoxy-2-[N-(1R,2S)-2-hydroxy-1-methyl-2- | |
| phenylethylcarbamoyl]propyl]-1-pyrrolidinyl]-1-[(S)-1-methylpropyl]-4-oxobutyl]-N-methylcarbamoyl}-2- | |
| methylpropylcarbamoyl]propyl](aminocarbonyloxy)}methyl)phenoxy}tetrahydro-2H-pyran-2-ylcarbonylamino]acetic acid | |
| 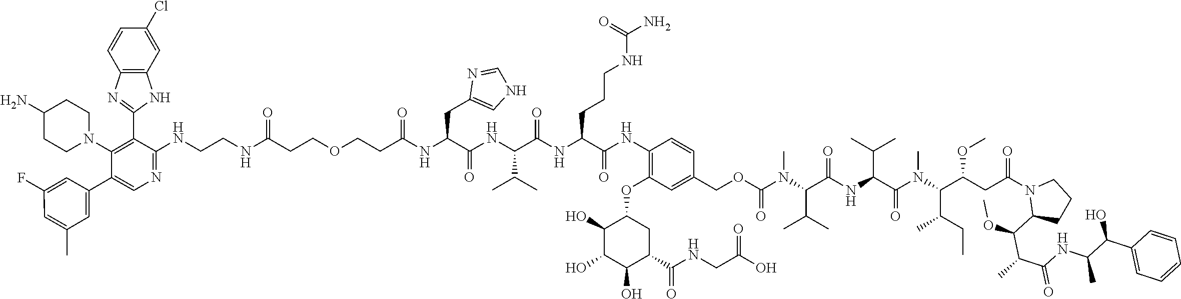 [SMILES: CC[C@H](C)[C@@H]([C@@H](CC(=O)N1CCC[C@H]1[C@H](OC)[C@@H](C)C(=O)N[C@H](C)[C@@H](O)c1ccccc1)OC)N(C)C(=O)[C@@H](NC(=O)[C@H](C(C)C)N(C)C(=O)OCc1ccc(NC(=O)[C@H](CCCNC(N)=O)NC(=O)[C@@H](NC(=O)[C@H](Cc2c[nH]cn2)NC(=O)CCOCCC(=O)NCCNc2ncc(-c3cc(C)cc(F)c3)c(N3CCC(N)CC3)c2-c2nc3ccc(Cl)cc3[nH]2)C(C)C)c(O[C@@H]2C[C@H](C(=O)NCC(=O)O)[C@@H](O)[C@H](O)[C@H]2O)c1)C(C)C] | |
| (Compound 212) | |
| [(2S,3S,4S,5R,6S)-3,4,5-trihydroxy-6-{2-[(S)-2-[(S)-2-[(S)-5-hydroxy-2-[3-(2-{N-2-14-(4-amino-1-piperidyl)-3-(6-chloro-1H- | 2107.0 |
| 1,3-benzimidazol-2-yl)-5-(3-fluoro-5-toly)-2-pyridylamino]ethylcarbamoyl}ethoxy)propionylamino]valerylamino]-3- | |
| methylbutyrylamino]-5-ureidovalerylamino]-5-({methyl[(S)-2-methyl-1-[N-(S)-1-{[(1S,2R)-2-methoxy-4-[(S)-2-[(1R,2R)-1- | |
| methoxy-2-[N-(1R,2S)-2-hydroxy-1-methyl-2-phenylethylcarbamoyl]propyl]-1-pyrrolidinyl]-1-[(S)-1-methylpropyl]-4- | |
| oxobutyl]-N-methylcarbamoyl}-2-methylpropylcarbamoyl]propyl](aminocarbonyloxy)}methyl)phenoxy}tetrahydro-2H-pyran- | |
| 2-ylcarbonylamino]acetic acid | |
| 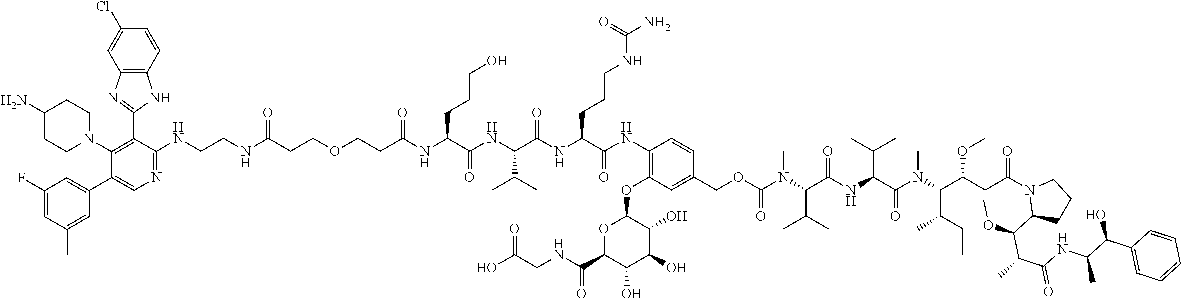 [SMILES: CC[C@H](C)[C@@H]([C@@H](CC(=O)N1CCC[C@H]1[C@H](OC)[C@@H](C)C(=O)N[C@H](C)[C@@H](O)c1ccccc1)OC)N(C)C(=O)[C@@H](NC(=O)[C@H](C(C)C)N(C)C(=O)OCc1ccc(NC(=O)[C@H](CCCNC(N)=O)NC(=O)[C@@H](NC(=O)[C@H](CCCO)NC(=O)CCOCCC(=O)NCCNc2ncc(-c3cc(C)cc(F)c3)c(N3CCC(N)CC3)c2-c2nc3cc(Cl)ccc3[nH]2)C(C)C)c(O[C@@H]2O[C@H](C(=O)NCC(=O)O)[C@@H](O)[C@H](O)[C@H]2O)c1)C(C)C] | |
| (Compound 213) | |
| [(2S,3S,4S,5R,6S)-3,4,5-trihydroxy-6-[5-({methyl[(S)-2-methyl-1-[N-(S)-1-{[(1S,2R)-2-methoxy-4-[(S)-2-[(1R,2R)-1- | 2113.0 |
| methoxy-2-[N-(1R,2S)-2-hydroxy-1-methyl-2-phenylethylcarbamoyl]propyl]-1-pyrrolidinyl]-1-[(S)-1-methylpropyl]-4- | |
| oxobutyl]-N-methylcarbamoyl}-2-methylpropylcarbamoyl]propyl](aminocarbonyloxy)}methyl)-2-[(S)-2-[(S)-3-methyl-2-[(S)- | |
| 2-[3-(2-{N-2-[4-(4-amino-1-piperidyl)-3-(6-chloro-1H-1,3-benzimidazol-2-yl)-5-(3-fluoro-5-tolyl)-2- | |
| pyridylamino]ethylcarbamoyl}ethoxy)propionylamino]propylsulfonylamino]butyrylamino]-5- | |
| ureidovalerylamino]phenoxy]tetrahydro-2H-pyran-2-ylcarbonylamino]acetic acid | |
| 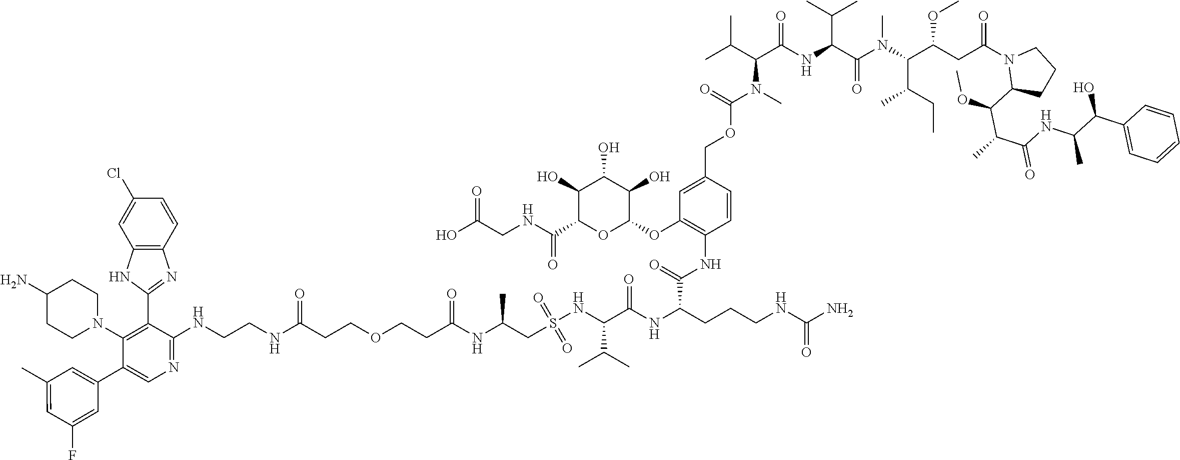 [SMILES: CC[C@H](C)[C@@H]([C@@H](CC(=O)N1CCC[C@H]1[C@H](OC)[C@@H](C)C(=O)N[C@H](C)[C@@H](O)c1ccccc1)OC)N(C)C(=O)[C@@H](NC(=O)[C@H](C(C)C)N(C)C(=O)OCc1ccc(NC(=O)[C@H](CCCNC(N)=O)NC(=O)[C@@H](NS(=O)(=O)C[C@H](C)NC(=O)CCOCCC(=O)NCCNc2ncc(-c3cc(C)cc(F)c3)c(N3CCC(N)CC3)c2-c2nc3ccc(Cl)cc3[nH]2)C(C)C)c(O[C@@H]2O[C@H](C(=O)NCC(=O)O)[C@@H](O)[C@H](O)[C@H]2O)c1)C(C)C] | |
| (Compound 214) | |
| 1-methyl 1-(S)-2-methyl-1-[N-(S)-1-{[(1S,2R)-2-methoxy-4-[(S)-2-[(1R,2R)-1-methoxy-2-[N-(1R,2S)-2-hydroxy-1-methyl-2- | 2076.1 |
| phenylethylcarbamoyl]propyl]-1-pyrrolidinyl]-1-[(S)-1-methylpropyl]-4-oxobutyl]-N-methylcarbamoyl}-2- | |
| methylpropylcarbamoyl]propyl{4-[(S)-2-[(S)-3-methyl-2-(3-{2-[N-methyl({N-2-[4-(4-amino-1-piperidyl)-3-(6-chloro-1H-1,3- | |
| benzimidazol-2-yl)-5-(3-fluoro-5-tolyl)-2- | |
| pyridylamino]ethylcarbamoyl}methyl)carbamoyl]ethoxy}propionylamino)butyrylamino]-5-ureidovalerylamino]-3- | |
| [(2S,3R,4S,5S,6S)-3,4,5-trihydroxy-6-[N-2-(dimethylamino)ethylcarbamoyl]tetrahydro-2H-pyran-2- | |
| yloxy]phenyl}methanecarbamate | |
| 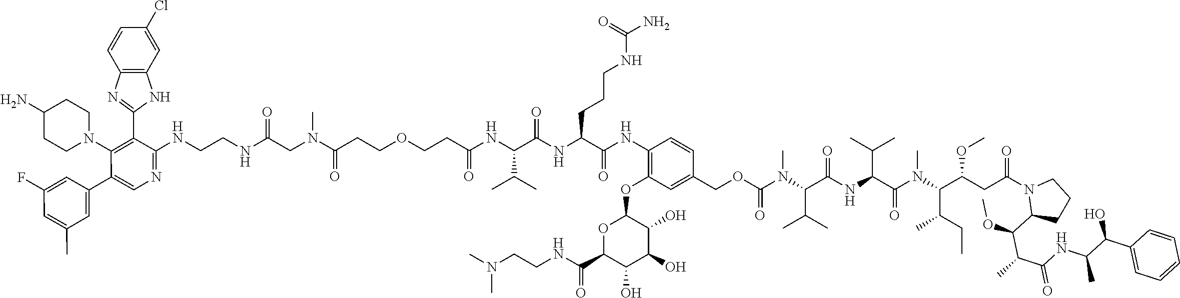 [SMILES: CC[C@H](C)[C@@H]([C@@H](CC(=O)N1CCC[C@H]1[C@H](OC)[C@@H](C)C(=O)N[C@H](C)[C@@H](O)c1ccccc1)OC)N(C)C(=O)[C@@H](NC(=O)[C@H](C(C)C)N(C)C(=O)OCc1ccc(NC(=O)[C@H](CCCNC(N)=O)NC(=O)[C@@H](NC(=O)CCOCCC(=O)N(C)CC(=O)NCCNc2ncc(-c3cc(C)cc(F)c3)c(N3CCC(N)CC3)c2-c2nc3ccc(Cl)cc3[nH]2)C(C)C)c(O[C@@H]2O[C@H](C(=O)NCCN(C)C)[C@@H](O)[C@H](O)[C@H]2O)c1)C(C)C] | |
| (Compound 215) | |
| {N-methyl[(2S,3S,4S,5R,6S)-3,4,5-trihydroxy-6-[5-({methyl[(S)-2-methyl-1-[N-(S)-1-{[(1S,2R)-2-methoxy-4-[(S)-2-[(1R,2R)- | 2006.0 |
| 1-methoxy-2-[N-(1R,2S)-2-hydroxy-1-methyl-2-phenylethylcarbamoyl]propyl]-1-pyrrolidinyl]-1-[(S)-1-methylpropyl]-4- | |
| oxobutyl]-N-methylcarbamoyl}-2-methylpropylcarbamoyl]propyl](aminocarbonyloxy)}methyl)-2-[(S)-2-[(S)-3-methyl-2-[3-(2- | |
| {N-2-[4-(4-amino-1-piperidyl)-3-(6-chloro-1H-1,3-benzimidazol-2-yl)-5-(3-fluoro-5-tolyl)-2- | |
| pyridylamino]ethylcarbamoyl}ethoxy)propionylamino]butyrylamino]-5-ureidovalerylamino]phenoxy]tetrahydro-2H-pyran-2- | |
| yl]carbonylamino}acetic acid | |
| 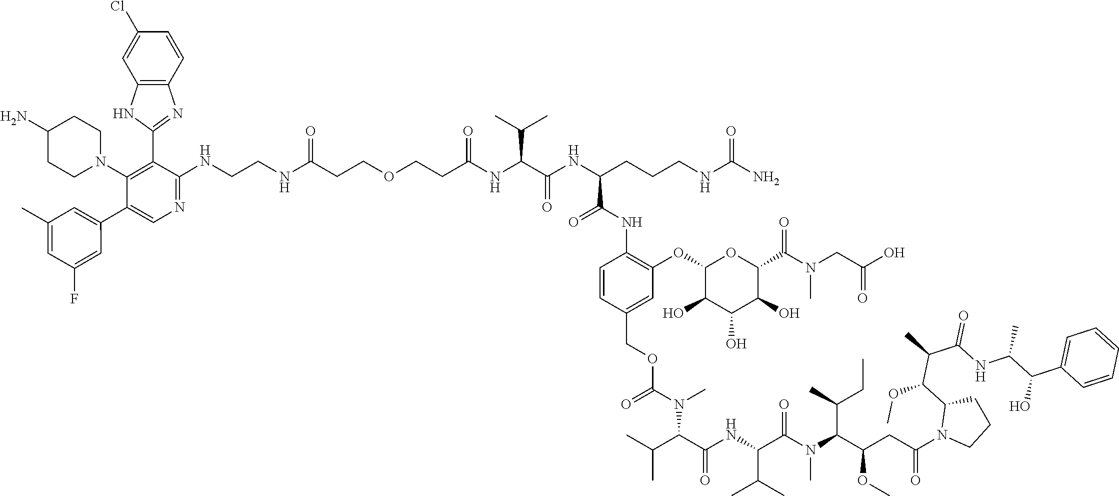 [SMILES: CC[C@H](C)[C@@H]([C@@H](CC(=O)N1CCC[C@H]1[C@H](OC)[C@@H](C)C(=O)N[C@H](C)[C@@H](O)c1ccccc1)OC)N(C)C(=O)[C@@H](NC(=O)[C@H](C(C)C)N(C)C(=O)OCc1ccc(NC(=O)[C@H](CCCNC(N)=O)NC(=O)[C@@H](NC(=O)CCOCCC(=O)NCCNc2ncc(-c3cc(C)cc(F)c3)c(N3CCC(N)CC3)c2-c2nc3ccc(Cl)cc3[nH]2)C(C)C)c(O[C@@H]2O[C@H](C(=O)N(C)CC(=O)O)[C@@H](O)[C@H](O)[C@H]2O)c1)C(C)C] | |
| (Compound 216) | |
| 1-methyl 1-(S)-2-methyl-1-[N-(S)-1-{[(1S,2R)-2-methoxy-4-[(S)-2-[(1R,2R)-1-methoxy-2-[N-(1R,2S)-2-hydroxy-1-methyl-2- | 1962.0 |
| phenylethylcarbamoyl]propyl]-1-pyrrolidinyl]-1-[(S)-1-methylpropyl]-4-oxobutyl]-N-methylcarbamoyl}-2- | |
| methylpropylcarbamoyl]propyl{4-[(S)-2-[(S)-3-methyl-2-[3-(2-{N-2-[4-(4-amino-1-piperidyl)-3-(6-chloro-1H-1,3- | |
| benzimidazol-2-yl)-5-(3-fluoro-5-tolyl)-2-pyridylamino]ethylcarbamoyl}ethoxy)propionylamino]butyrylamino]-5- | |
| ureidovalerylamino]-3-[(2S,3R,4S,5S,6S)-3,4,5-trihydroxy-6-(N,N-dimethylcarbamoyl)tetrahydro-2H-pyran-2- | |
| yloxy]phenyl}methanecarbamate | |
| 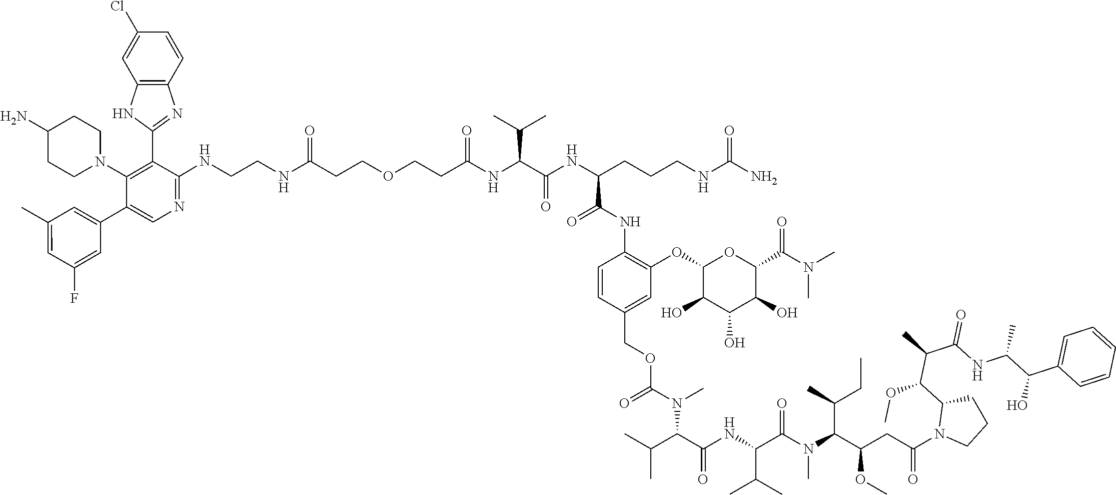 [SMILES: CC[C@H](C)[C@@H]([C@@H](CC(=O)N1CCC[C@H]1[C@H](OC)[C@@H](C)C(=O)N[C@H](C)[C@@H](O)c1ccccc1)OC)N(C)C(=O)[C@@H](NC(=O)[C@H](C(C)C)N(C)C(=O)OCc1ccc(NC(=O)[C@H](CCCNC(N)=O)NC(=O)[C@@H](NC(=O)CCOCCC(=O)NCCNc2ncc(-c3cc(C)cc(F)c3)c(N3CCC(N)CC3)c2-c2nc3ccc(Cl)cc3[nH]2)C(C)C)c(O[C@@H]2O[C@H](C(=O)N(C)C)[C@@H](O)[C@H](O)[C@H]2O)c1)C(C)C] | |
| (Compound 217) | |
| 2-{N-methyl[(2S,3S,4S,5R,6S)-3,4,5-trihydroxy-6-[5-({methyl[(S)-2-methyl-1-[N-(S)-1-{[(1S,2R)-2-methoxy-4-[(S)-2- | 2056.0 |
| [(1R,2R)-1-methoxy-2-[N-(1R,2S)-2-hydroxy-1-methyl-2-phenylethylcarbamoyl]propyl]-1-pyrrolidinyl]-1-[(S)-1- | |
| methylpropyl]-4-oxobutyl]-N-methylcarbamoyl}-2-methylpropylcarbamoyl]propyl](aminocarbonyloxy)}methyl)-2-[(S)-2-[(S)- | |
| 3-methyl-2-[3-(2-{N-2-[4-(4-amino-1-piperidyl)-3-(6-chloro-1H-1,3-benzimidazol-2-yl)-5-(3-fluoro-5-tolyl)-2- | |
| pyridylamino]ethylcarbamoyl}ethoxy)propionylamino]butyrylamino]-5-ureidovalerylamino]phenoxy]tetrahydro-2H-pyran-2- | |
| yl]carbonylamino}ethanesulfonic acid | |
| 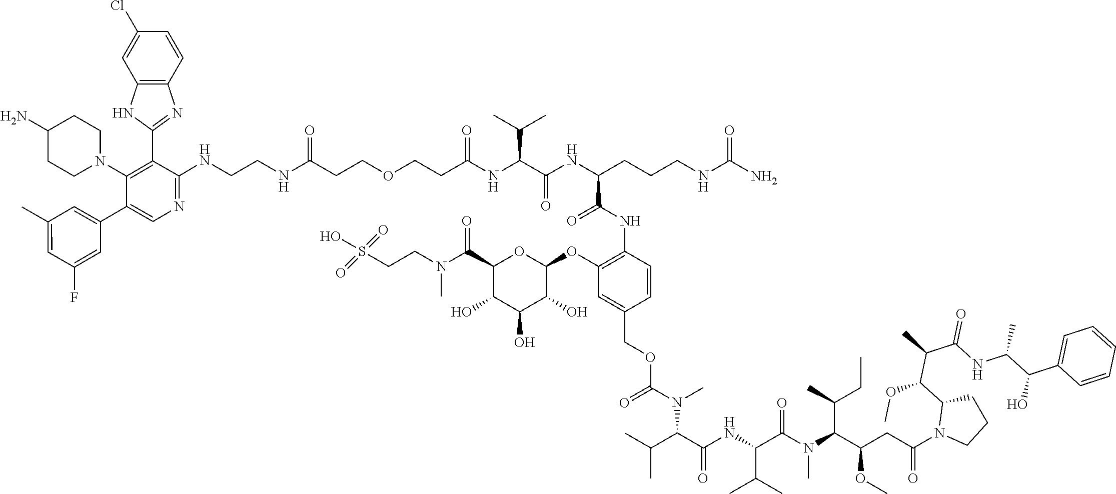 [SMILES: CC[C@H](C)[C@@H]([C@@H](CC(=O)N1CCC[C@H]1[C@H](OC)[C@@H](C)C(=O)N[C@H](C)[C@@H](O)c1ccccc1)OC)N(C)C(=O)[C@@H](NC(=O)[C@H](C(C)C)N(C)C(=O)OCc1ccc(NC(=O)[C@H](CCCNC(N)=O)NC(=O)[C@@H](NC(=O)CCOCCC(=O)NCCNc2ncc(-c3cc(C)cc(F)c3)c(N3CCC(N)CC3)c2-c2nc3ccc(Cl)cc3[nH]2)C(C)C)c(O[C@@H]2O[C@H](C(=O)N(C)CCS(=O)(=O)O)[C@@H](O)[C@H](O)[C@H]2O)c1)C(C)C] | |
| (Compound 218) | |
| {N-methyl[(2S,3S,4S,5R,6S)-3,4,5-trihydroxy-6-(2-[(S)-2-[(S)-2-[(S)-5-hydroxy-2-[3-(2-{N-2-14-(4-amino-1-piperidyl)-3-(6- | 2121.1 |
| chloro-1H-1,3-benzimidazol-2-yl)-5-(3-fluoro-5-tolyl)-2-pyridylamino]ethylcarbamoyl}ethoxy)propionylamino]valerylamino]- | |
| 3-methylbutyrylamino]-5-ureidovalerylamino]-5-({methyl[(S)-2-methyl-1-[N-(S)-1-{[(1S,2R)-2-methoxy-4-[(S)-2-[(1R,2R)-1- | |
| methoxy-2-[N-(1R,2S)-2-hydroxy-1-methyl-2-phenylethylcarbamoyl]propyl]-1-pyrrolidinyl]-1-[(S)-1-methylpropyl]-4- | |
| oxobutyl]-N-methylcarbamoyl}-2-methylpropylcarbamoyl]propyl](aminocarbonyloxy)}methyl)phenoxy}tetrahydro-2H-pyran- | |
| 2-yl]carbonylamino}acetic acid | |
| 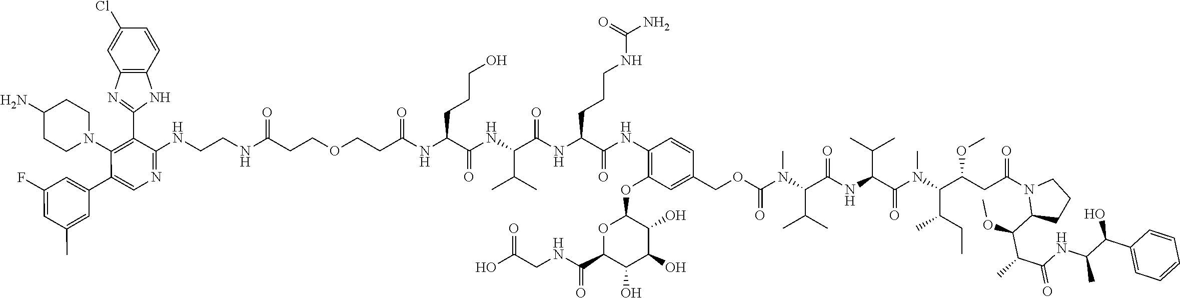 [SMILES: CC[C@H](C)[C@@H]([C@@H](CC(=O)N1CCC[C@H]1[C@H](OC)[C@@H](C)C(=O)N[C@H](C)[C@@H](O)c1ccccc1)OC)N(C)C(=O)[C@@H](NC(=O)[C@H](C(C)C)N(C)C(=O)OCc1ccc(NC(=O)[C@H](CCCNC(N)=O)NC(=O)[C@@H](NC(=O)[C@H](CCCO)NC(=O)CCOCCC(=O)NCCNc2ncc(-c3cc(C)cc(F)c3)c(N3CCC(N)CC3)c2-c2nc3cc(Cl)ccc3[nH]2)C(C)C)c(O[C@@H]2O[C@H](C(=O)NCC(=O)O)[C@@H](O)[C@H](O)[C@H]2O)c1)C(C)C] | |
| (Compound 219) | |
| {N-methyl[(2S,3S,4S,5R,6S)-3,4,5-trihydroxy-6-[5-({methyl[(S)-2-methyl-1-[N-(S)-1-{[(18,2R)-2-methoxy-4-[(S)-2-[(1R,2R)- | 2127.0 |
| 1-methoxy-2-[N-(1R,2S)-2-hydroxy-1-methyl-2-phenylethylcarbamoyl]propyl]-1-pyrrolidinyl]-1-[(S)-1-methylpropyl]-4- | |
| oxobutyl]-N-methylcarbamoyl}-2-methylpropylcarbamoyl]propyl](aminocarbonyloxy)}methyl)-2-[(S)-2-[(S)-3-methyl-2-[(S)- | |
| 2-[3-(2-{N-2-[4-(4-amino-1-piperidyl)-3-(6-chloro-1H-1,3-benzimidazol-2-y])-5-(3-fluoro-5-tolyl)-2- | |
| pyridylamino]ethylcarbamoyl}ethoxy)propionylamino]propylsulfonylamino]butyrylamino]-5- | |
| ureidovalerylamino]phenoxy]tetrahydro-2H-pyran-2-yl]carbonylamino}acetic acid | |
| 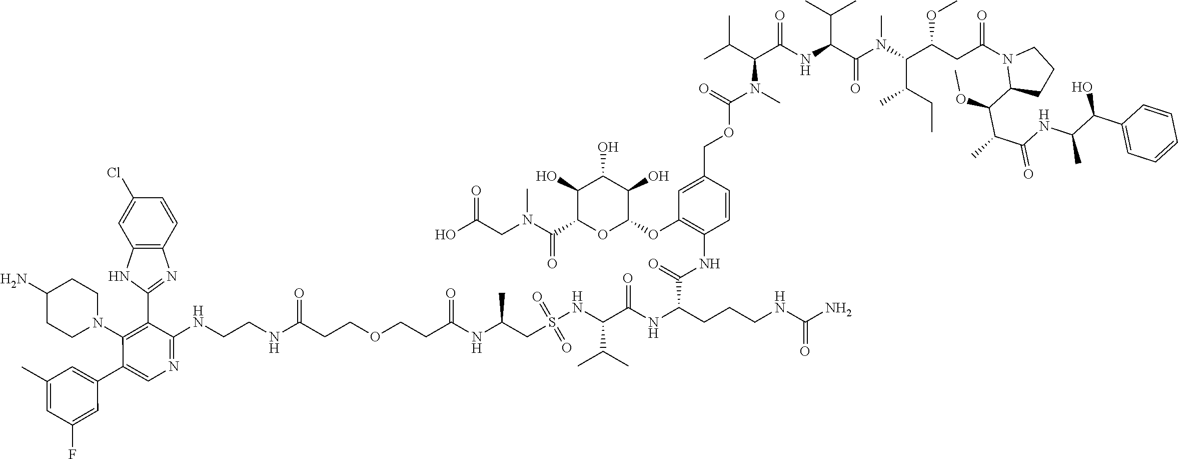 [SMILES: CC[C@H](C)[C@@H]([C@@H](CC(=O)N1CCC[C@H]1[C@H](OC)[C@@H](C)C(=O)N[C@H](C)[C@@H](O)c1ccccc1)OC)N(C)C(=O)[C@@H](NC(=O)[C@H](C(C)C)N(C)C(=O)OCc1ccc(NC(=O)[C@H](CCCNC(N)=O)NC(=O)[C@@H](NS(=O)(=O)C[C@H](C)NC(=O)CCOCCC(=O)NCCNc2ncc(-c3cc(C)cc(F)c3)c(N3CCC(N)CC3)c2-c2nc3ccc(Cl)cc3[nH]2)C(C)C)c(O[C@@H]2O[C@H](C(=O)N(C)CC(=O)O)[C@@H](O)[C@H](O)[C@H]2O)c1)C(C)C] | |
| (Compound 220) | |
| {N-methyl[(2S,3S,4S,5R,6S)-3,4,5-trihydroxy-6-[5-({methyl[(S)-2-methyl-1-[N-(S)-1-{[(1S,2R)-2-methoxy-4-[(S)-2-[(1R,2R)- | 2040.9 |
| 1-methoxy-2-[N-(1R,2S)-2-hydroxy-1-methyl-2-phenylethylcarbamoyl]propyl]-1-pyrrolidinyl]-1-[(S)-1-methylpropyl]-4- | |
| oxobutyl]-N-methylcarbamoyl}-2-methylpropylcarbamoyl]propyl](aminocarbonyloxy)}methyl)-2-[(S)-2-[(S)-3-methyl-2-[(S)- | |
| 2-[3-(2-{N-2-[4-(4-amino-1-piperidyl)-3-(6-chloro-1H-1,3-benzimidazol-2-y])-5-(3-fluoro-5-tolyl)-2- | |
| pyridylamino]ethylcarbamoyl}ethoxy)propionylamino]propylsulfonylamino]butyrylamino]propionylamino]phenoxy]tetrahydro- | |
| 2H-pyran-2-yl]carbonylamino}acetic acid | |
| 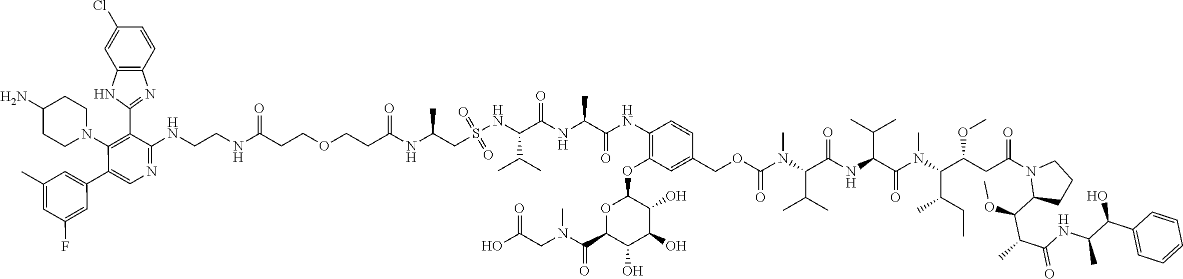 [SMILES: CC[C@H](C)[C@@H]([C@@H](CC(=O)N1CCC[C@H]1[C@H](OC)[C@@H](C)C(=O)N[C@H](C)[C@@H](O)c1ccccc1)OC)N(C)C(=O)[C@@H](NC(=O)[C@H](C(C)C)N(C)C(=O)OCc1ccc(NC(=O)[C@H](C)NC(=O)[C@@H](NS(=O)(=O)C[C@H](C)NC(=O)CCOCCC(=O)NCCNc2ncc(-c3cc(C)cc(F)c3)c(N3CCC(N)CC3)c2-c2nc3ccc(Cl)cc3[nH]2)C(C)C)c(O[C@@H]2O[C@H](C(=O)N(C)CC(=O)O)[C@@H](O)[C@H](O)[C@H]2O)c1)C(C)C] | |
| (Compound 221) | |
| 1-methyl 1-(S)-2-methyl-1-[N-(S)-1-{[(1S,2R)-2-methoxy-4-[(S)-2-[(1R,2R)-1-methoxy-2-[N-(1R,2S)-2-hydroxy-1-methyl-2- | 2003.0 |
| phenylethylcarbamoyl]propyl]-1-pyrrolidinyl]-1-[(S)-1-methylpropyl]-4-oxobutyl]-N-methylcarbamoyl}-2- | |
| methylpropylcarbamoyl]propyl{4-[(S)-2-[(S)-3-methyl-2-[3-(2-{N-2-[4-(4-amino-1-piperidyl)-3-(6-chloro-1H-1,3- | |
| benzimidazol-2-yl)-5-(3-fluoro-5-tolyl)-2-pyridylamino]ethylcarbamoyl}ethoxy)propionylamino]butyrylamino]-5- | |
| ureidovalerylamino]-3-[(2S,3R,4S,5S,6S)-3,4,5-trihydroxy-6-[(1-piperaziny])carbonyl]tetrahydro-2H-pyran-2- | |
| yloxy]phenyl} methanecarbamate | |
| 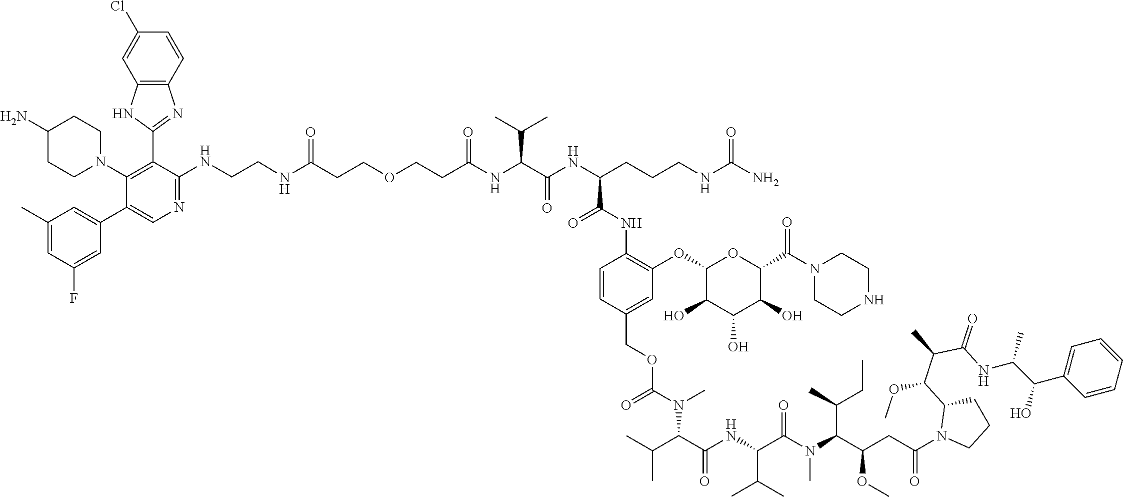 [SMILES: CC[C@H](C)[C@@H]([C@@H](CC(=O)N1CCC[C@H]1[C@H](OC)[C@@H](C)C(=O)N[C@H](C)[C@@H](O)c1ccccc1)OC)N(C)C(=O)[C@@H](NC(=O)[C@H](C(C)C)N(C)C(=O)OCc1ccc(NC(=O)[C@H](CCCNC(N)=O)NC(=O)[C@@H](NC(=O)CCOCCC(=O)NCCNc2ncc(-c3cc(C)cc(F)c3)c(N3CCC(N)CC3)c2-c2nc3ccc(Cl)cc3[nH]2)C(C)C)c(O[C@@H]2O[C@H](C(=O)N3CCNCC3)[C@@H](O)[C@H](O)[C@H]2O)c1)C(C)C] | |
| (Compound 222) | |
| 1-methyl 1-(S)-2-methyl-1-[N-(S)-1-{[(1S,2R)-2-methoxy-4-[(S)-2-[(1R,2R)-1-methoxy-2-[N-(1R,2S)-2-hydroxy-1-methyl-2- | 2004.0 |
| phenylethylcarbamoyl]propyl]-1-pyrrolidinyl]-1-[(S)-1-methylpropyl]-4-oxobutyl]-N-methylcarbamoyl}-2- | |
| methylpropylcarbamoyl]propyl{4-[(S)-2-[(S)-3-methyl-2-[3-(2-{N-2-[4-(4-amino-1-piperidyl)-3-(6-chloro-1H-1,3- | |
| benzimidazol-2-yl)-5-(3-fluoro-5-tolyl)-2-pyridylamino]ethylcarbamoyl}ethoxy)propionylamino]butyrylamino]-5- | |
| ureidovalerylamino]-3-[(2S,3R,4S,5S,6S)-3,4,5-trihydroxy-6-morpholinocarbonyltetrahydro-2H-pyran-2- | |
| yloxy]phenyl}methanecarbamate | |
| 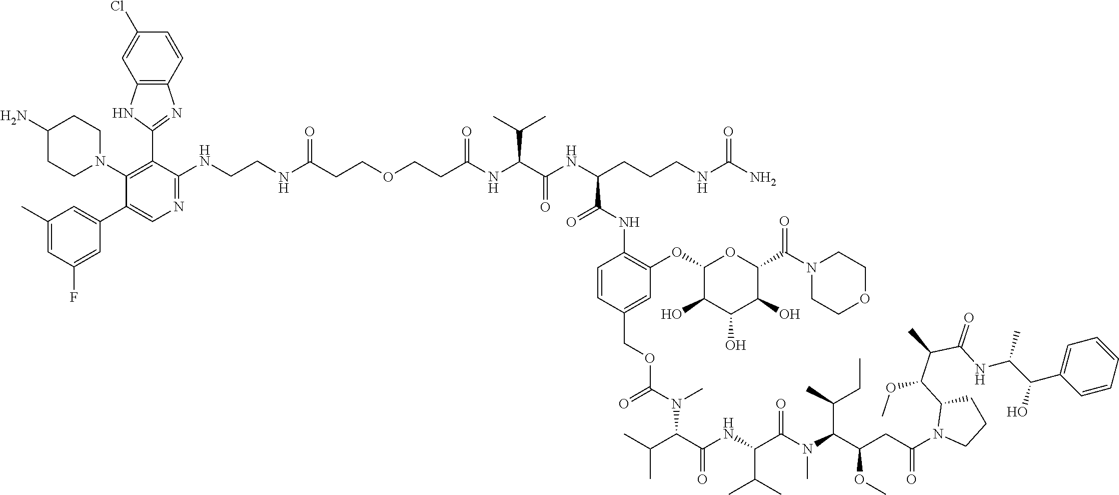 [SMILES: CC[C@H](C)[C@@H]([C@@H](CC(=O)N1CCC[C@H]1[C@H](OC)[C@@H](C)C(=O)N[C@H](C)[C@@H](O)c1ccccc1)OC)N(C)C(=O)[C@@H](NC(=O)[C@H](C(C)C)N(C)C(=O)OCc1ccc(NC(=O)[C@H](CCCNC(N)=O)NC(=O)[C@@H](NC(=O)CCOCCC(=O)NCCNc2ncc(-c3cc(C)cc(F)c3)c(N3CCC(N)CC3)c2-c2nc3ccc(Cl)cc3[nH]2)C(C)C)c(O[C@@H]2O[C@H](C(=O)N3CCOCC3)[C@@H](O)[C@H](O)[C@H]2O)c1)C(C)C] | |
| (Compound 223) | |
| 1-methyl 1-(S)-2-methyl-1-[N-(S)-1-{[(1S,2R)-2-methoxy-4-[(S)-2-[(1R,2R)-1-methoxy-2-[N-(1R,2S)-2-hydroxy-1-methyl-2- | 2266.0 |
| phenylethylcarbamoyl]propyl]-1-pyrrolidinyl]-1-[(S)-1-methylpropyl]-4-oxobutyl]-N-methylcarbamoyl}-2- | |
| methylpropylcarbamoyl]propyl {4-[(S)-2-[(S)-3-methyl-2-[3-(2-{N-2-[4-(4-amino-1-piperidyl)-3-(6-chloro-1H-1,3- | |
| benzimidazol-2-yl)-5-(3-fluoro-5-tolyl)-2-pyridylamino]ethylcarbamoyl}ethoxy)propionylamino]butyrylamino]-5- | |
| ureidovalerylamino]-3-[(2S,3R,4S,5S,6S)-3,4,5-trihydroxy-6-{N-2-[3-(N-2- | |
| naphthylsulfonylcarbamoyl)propionylamino]ethylcarbamoyl}tetrahydro-2H-pyran-2-yloxy]phenyl}methanecarbamate | |
| 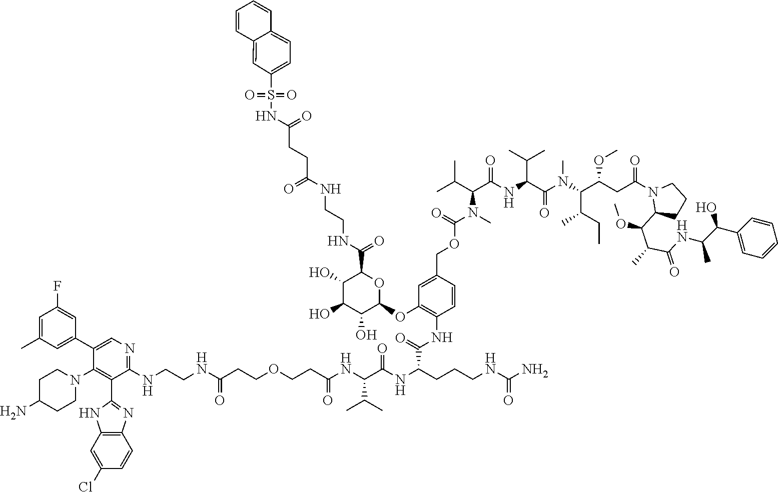 [SMILES: CC[C@H](C)[C@@H]([C@@H](CC(=O)N1CCC[C@H]1[C@H](OC)[C@@H](C)C(=O)N[C@H](C)[C@@H](O)c1ccccc1)OC)N(C)C(=O)[C@@H](NC(=O)[C@H](C(C)C)N(C)C(=O)OCc1ccc(NC(=O)[C@H](CCCNC(N)=O)NC(=O)[C@@H](NC(=O)CCOCCC(=O)NCCNc2ncc(-c3cc(C)cc(F)c3)c(N3CCC(N)CC3)c2-c2nc3ccc(Cl)cc3[nH]2)C(C)C)c(O[C@@H]2O[C@H](C(=O)NCCNC(=O)CCC(=O)NS(=O)(=O)c3ccc4ccccc4c3)[C@@H](O)[C@H](O)[C@H]2O)c1)C(C)C] | |
| (Compound 224) | |
| 1-methyl 1-(S)-2-methyl-1-[N-(S)-1-{[(1S,2R)-2-methoxy-4-[(S)-2-[(1R,2R)-1-methoxy-2-[N-(1R,2S)-2-hydroxy-1-methyl-2- | 2376.1 |
| phenylethylcarbamoyl]propyl]-1-pyrrolidinyl]-1-[(S)-1-methylpropyl]-4-oxobutyl]-N-methylcarbamoyl}-2- | |
| methylpropylcarbamoyl]propyl {4-[(S)-2-[(S)-3-methyl-2-[3-(2-{N-2-[4-(4-amino-1-piperidyl)-3-(6-chloro-1H-1,3- | |
| benzimidazol-2-yl)-5-(3-fluoro-5-tolyl)-2-pyridylamino]ethylcarbamoyl}ethoxy)propionylamino]butyrylamino]-5- | |
| ureidovalerylamino]-3-[(2S,3R,4S,5S,6S)-3,4,5-trihydroxy-6-(N-2-{N-5-[5-(p- | |
| iodophenyl)valerylamino]pentylcarbamoyl}ethylcarbamoyl)tetrahydro-2H-pyran-2-yloxy]phenyl}methanecarbamate | |
| 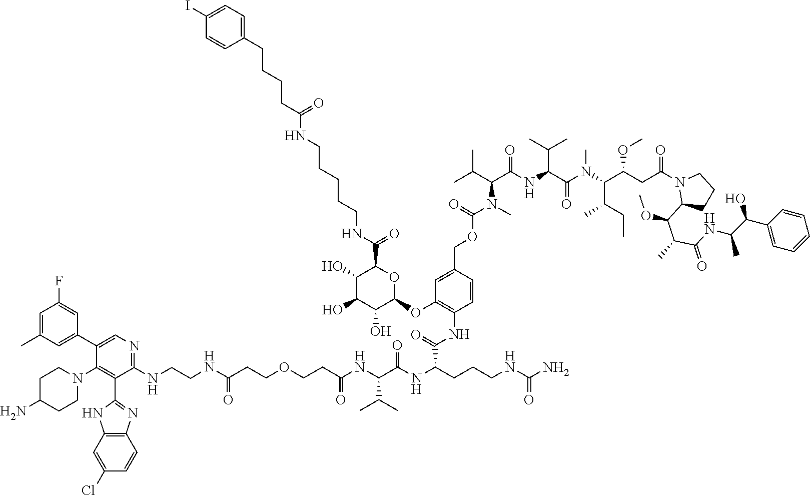 [SMILES: CC[C@H](C)[C@@H]([C@@H](CC(=O)N1CCC[C@H]1[C@H](OC)[C@@H](C)C(=O)N[C@H](C)[C@@H](O)c1ccccc1)OC)N(C)C(=O)[C@@H](NC(=O)[C@H](C(C)C)N(C)C(=O)OCc1ccc(NC(=O)[C@H](CCCNC(N)=O)NC(=O)[C@@H](NC(=O)CCOCCC(=O)NCCNc2ncc(-c3cc(C)cc(F)c3)c(N3CCC(N)CC3)c2-c2nc3ccc(Cl)cc3[nH]2)C(C)C)c(O[C@@H]2O[C@H](C(=O)NCCCCCNC(=O)CCCCc3ccc(I)cc3)[C@@H](O)[C@H](O)[C@H]2O)c1)C(C)C] | |
| (Compound 225) | |
| 1-methyl 1-(S)-2-methyl-1-[N-(S)-1-{[(1S,2R)-2-methoxy-4-[(S)-2-[(1R,2R)-1-methoxy-2-[N-(1R,2S)-2-hydroxy-1-methyl-2- | 2017.0 |
| phenylethylcarbamoyl]propyl]-1-pyrrolidinyl]-1-[(S)-1-methylpropyl]-4-oxobutyl]-N-methylcarbamoyl}-2- | |
| methylpropylcarbamoyl]propyl {4-[(S)-2-[(S)-3-methyl-2-[3-(2-{N-2-[4-(4-amino-1-piperidyl)-3-(6-chloro-1H-1,3- | |
| benzimidazol-2-yl)-5-(3-fluoro-5-tolyl)-2-pyridylamino]ethylcarbamoyl}ethoxy)propionylamino]butyrylamino]-5- | |
| ureidovalerylamino]-3-[(2S,3R,4S,5S,6S)-3,4,5-trihydroxy-6-[(4-methyl-1-piperaziny])carbonyl]tetrahydro-2H-pyran-2- | |
| yloxy]phenyl}methanecarbamate | |
| 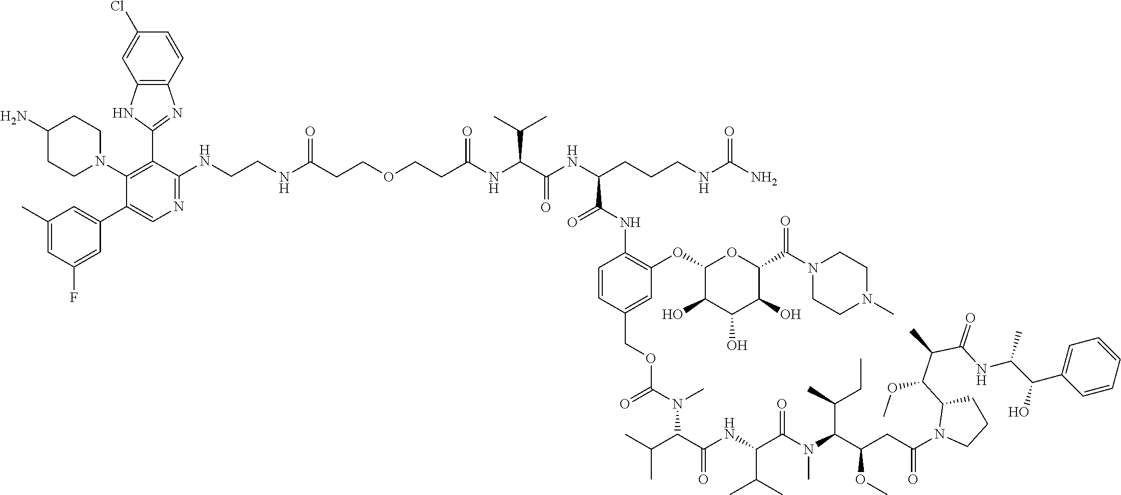 | |
| (Compound 226) | |
| (4-{[(2S,3S,4S,5R,6S)-3,4,5-trihydroxy-6-[5-({methyl[(S)-2-methyl-1-[N-(S)-1-{[(1S,2R)-2-methoxy-4-[(S)-2-[(1R,2R)-1- | 2061.0 |
| methoxy-2-[N-(1R,2S)-2-hydroxy-1-methyl-2-phenylethylcarbamoyl]propyl]-1-pyrrolidinyl]-1-[(S)-1-methylpropyl]-4- | |
| oxobutyl]-N-methylcarbamoyl}-2-methylpropylcarbamoyl]propyl](aminocarbonyloxy)}methyl)-2-[(S)-2-[(S)-3-methyl-2-[3-(2- | |
| (N-2-[4-(4-amino-1-piperidyl)-3-(6-chloro-1H-1,3-benzimidazol-2-yl)-5-(3-fluoro-5-tolyl)-2- | |
| pyridylamino]ethylcarbamoyl}ethoxy)propionylamino]butyrylamino]-5-ureidovalerylamino]phenoxy]tetrahydro-2H-pyran-2- | |
| yllcarbonyl}-1-piperazinyl)acetic acid | |
| 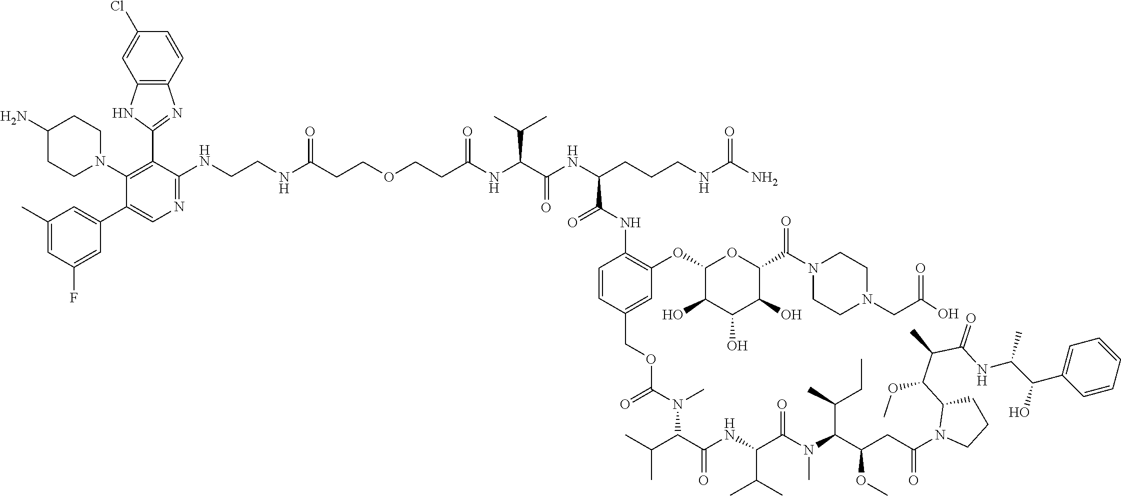 | |
| (Compound 227) | |
| 1-methyl 1-(S)-2-methyl-1-[N-(S)-1-{[(1S,2R)-2-methoxy-4-[(S)-2-[(1R,2R)-1-methoxy-2-[N-(1R,2S)-2-hydroxy-1-methyl-2- | 2002.0 |
| phenylethylcarbamoyl]propyl]-1-pyrrolidinyl]-1-[(S)-1-methylpropyl]-4-oxobutyl]-N-methylcarbamoyl}-2- | |
| methylpropylcarbamoyl]propyl {4-[(S)-2-[(S)-3-methyl-2-[3-(2-{N-2-[4-(4-amino-1-piperidyl)-3-(6-chloro-1H-1,3- | |
| benzimidazol-2-yl)-5-(3-fluoro-5-tolyl)-2-pyridylamino]ethylcarbamoyl}ethoxy)propionylamino]butyrylamino]-5- | |
| ureidovalerylamino]-3-[(2S,3R,4S,5S,6S)-3,4,5-trihydroxy-6-piperidinocarbonyltetrahydro-2H-pyran-2- | |
| yloxy]phenyl}methanecarbamate | |
| 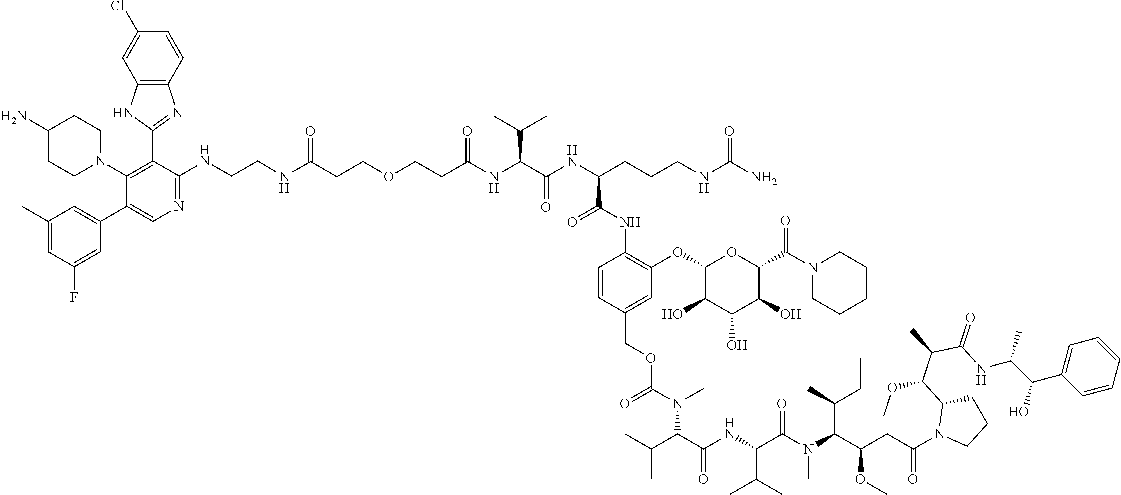 | |
| (Compound 228) | |
| 1-methyl 1-(S)-2-methyl-1-[N-(S)-1-{[(1S,2R)-2-methoxy-4-[(S)-2-[(1R,2R)-1-methoxy-2-[N-(1R,2S)-2-hydroxy-1-methyl-2- | 2047.0 |
| phenylethylcarbamoyl]propyl]-1-pyrrolidinyl]-1-[(S)-1-methylpropyl]-4-oxobutyl]-N-methylcarbamoyl}-2- | |
| methylpropylcarbamoyl]propyl {4-[(S)-2-[(S)-3-methyl-2-[3-(2-{N-2-[4-(4-amino-1-piperidyl)-3-(6-chloro-1H-1,3- | |
| benzimidazol-2-yl)-5-(3-fluoro-5-tolyl)-2-pyridylamino]ethylcarbamoyl}ethoxy)propionylamino]butyrylamino]-5- | |
| ureidovalerylamino]-3-[(2S,3R,4S,5S,6S)-3,4,5-trihydroxy-6-{[4-(2-hydroxyethyl)-1-piperaziny]]carbonyl}tetrahydro-2H- | |
| pyran-2-yloxy]phenyl}methanecarbamate | |
| 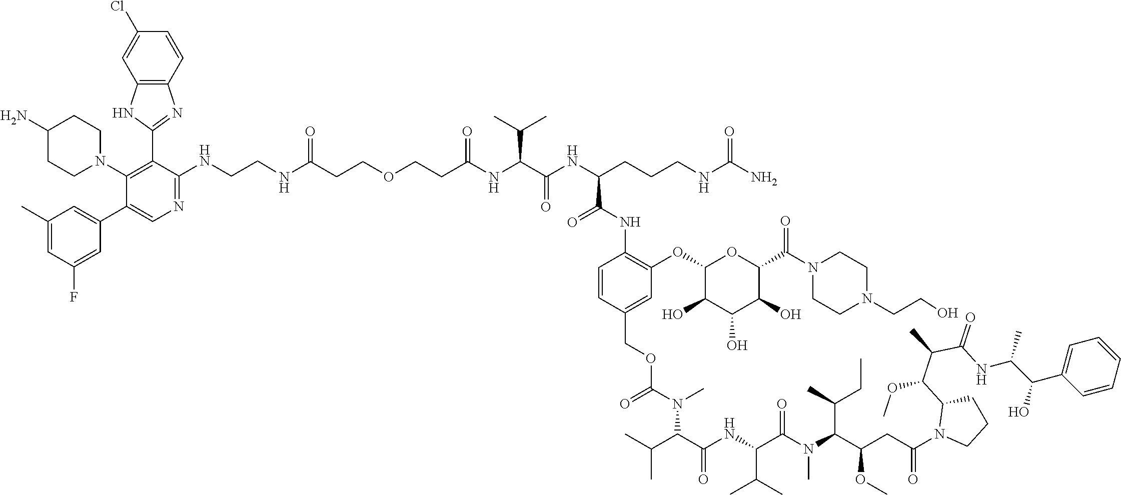 | |
| (Compound 229) | |
| 3-{2-[(2S,3S,4S,5R,6S)-3,4,5-trihydroxy-6-[5-({methyl[(S)-2-methyl-1-[N-(S)-1-{[(1S,2R)-2-methoxy-4-[(S)-2-[(1R,2R)-1- | 2050.0 |
| methoxy-2-[N-(1R,2S)-2-hydroxy-1-methyl-2-phenylethylcarbamoyl]propyl]-1-pyrrolidinyl]-1-[(S)-1-methylpropyl]-4- | |
| oxobutyl]-N-methylcarbamoyl}-2-methylpropylcarbamoyl]propyl](aminocarbonyloxy)}methyl)-2-[(S)-2-[(S)-3-methyl-2-[3-(2- | |
| (N-2-[4-(4-amino-1-piperidyl)-3-(6-chloro-1H-1,3-benzimidazol-2-yl)-5-(3-fluoro-5-tolyl)-2- | |
| pyridylamino]ethylcarbamoyl}ethoxy)propionylamino]butyrylamino]-5-ureidovalerylamino]phenoxy]tetrahydro-2H-pyran-2- | |
| ylcarbonylamino]ethoxy}propionic acid | |
| 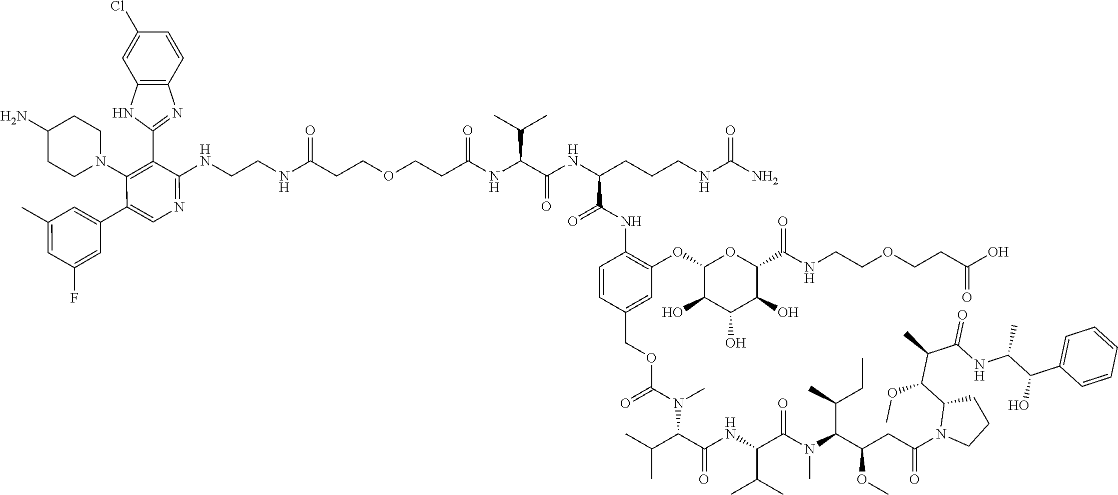 [SMILES: CC[C@H](C)[C@@H]([C@@H](CC(=O)N1CCC[C@H]1[C@H](OC)[C@@H](C)C(=O)N[C@H](C)[C@@H](O)c1ccccc1)OC)N(C)C(=O)[C@@H](NC(=O)[C@H](C(C)C)N(C)C(=O)OCc1ccc(NC(=O)[C@H](CCCNC(N)=O)NC(=O)[C@@H](NC(=O)CCOCCC(=O)NCCNc2ncc(-c3cc(C)cc(F)c3)c(N3CCC(N)CC3)c2-c2nc3ccc(Cl)cc3[nH]2)C(C)C)c(O[C@@H]2O[C@H](C(=O)NCCOCCC(=O)O)[C@@H](O)[C@H](O)[C@H]2O)c1)C(C)C] | |
| (Compound 230) | |
| 3-(2-{N-methyl[(28,38,4S,5R,6S)-3,4,5-trihydroxy-6-[5-({methyl[(S)-2-methyl-1-[N-(S)-1-{[(1S,2R)-2-methoxy-4-[(S)-2- | 2064.0 |
| [(1R,2R)-1-methoxy-2-[N-(1R,2S)-2-hydroxy-1-methyl-2-phenylethylcarbamoyl]propyl]-1-pyrrolidinyl]-1-[(S)-1- | |
| methylpropyl]-4-oxobutyl]-N-methylcarbamoyl}-2-methylpropylcarbamoyl]propyl](aminocarbonyloxy)}methyl)-2-[(S)-2-[(S)- | |
| 3-methyl-2-[3-(2-{N-2-[4-(4-amino-1-piperidyl)-3-(6-chloro-1H-1,3-benzimidazol-2-yl)-5-(3-fluoro-5-tolyl)-2- | |
| pyridylamino]ethylcarbamoyl}ethoxy)propionylamino]butyrylamino]-5-ureidovalerylamino]phenoxy]tetrahydro-2H-pyran-2- | |
| yl]carbonylamino}ethoxy)propionic acid | |
| 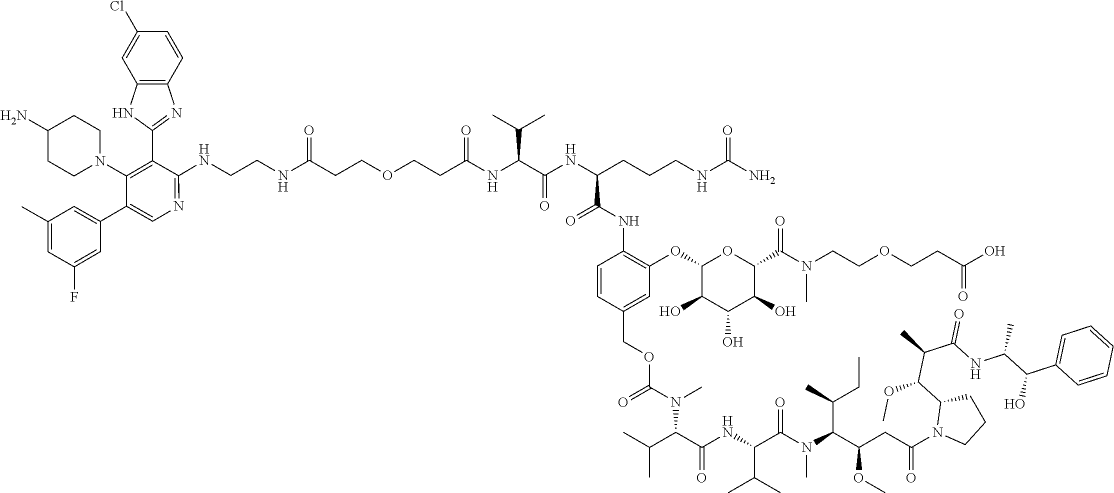 [SMILES: CC[C@H](C)[C@@H]([C@@H](CC(=O)N1CCC[C@H]1[C@H](OC)[C@@H](C)C(=O)N[C@H](C)[C@@H](O)c1ccccc1)OC)N(C)C(=O)[C@@H](NC(=O)[C@H](C(C)C)N(C)C(=O)OCc1ccc(NC(=O)[C@H](CCCNC(N)=O)NC(=O)[C@@H](NC(=O)CCOCCC(=O)NCCNc2ncc(-c3cc(C)cc(F)c3)c(N3CCC(N)CC3)c2-c2nc3ccc(Cl)cc3[nH]2)C(C)C)c(O[C@@H]2O[C@H](C(=O)N(C)CCOCCC(=O)O)[C@@H](O)[C@H](O)[C@H]2O)c1)C(C)C] | |
| (Compound 231) | |
| 1-methyl 1-(S)-2-methyl-1-[N-(S)-1-{[(1S,2R)-2-methoxy-4-[(S)-2-[(1R,2R)-1-methoxy-2-[N-(1R,2S)-2-hydroxy-1-methyl-2- | 1989.0 |
| phenylethylcarbamoyl]propyl]-1-pyrrolidinyl]-1-[(S)-1-methylpropyl]-4-oxobutyl]-N-methylcarbamoyl}-2- | |
| methylpropylcarbamoyI]propyl {3-[(28,3R,48,5S,6S)-6-[(3-amino-1-azetidinyl)carbonyl]-3,4,5-trihydroxytetrahydro-2H-pyran- | |
| 2-yloxy]-4-[(S)-2-[(S)-3-methyl-2-[3-(2-{N-2-[4-(4-amino-1-piperidyl)-3-(6-chloro-1H-1,3-benzimidazol-2-yl)-5-(3-fluoro-5- | |
| tolyl)-2-pyridylamino]ethylcarbamoyl}ethoxy)propionylamino]butyrylamino]-5-ureidovalerylamino]phenyl}methanecarbamate | |
| 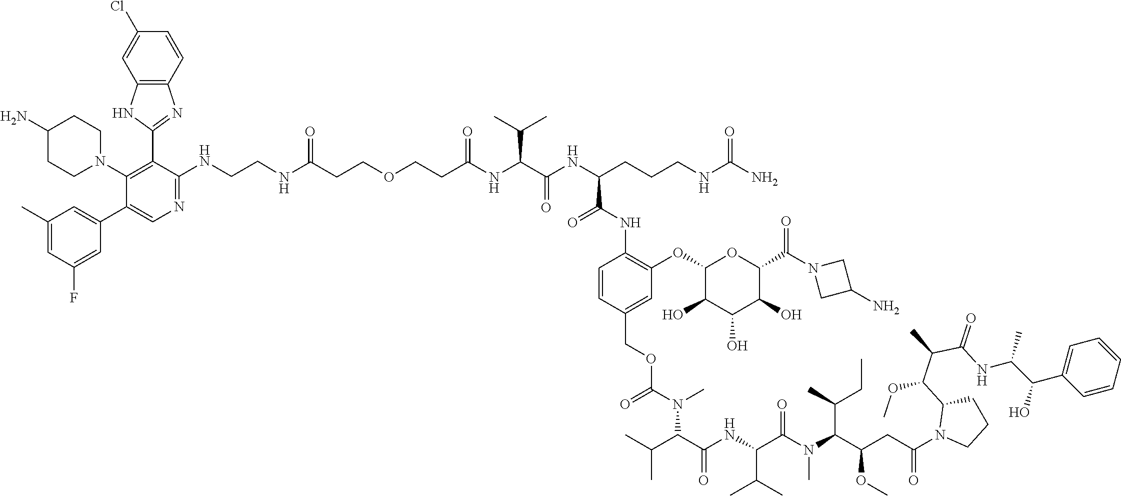 | |
| (Compound 232) | |
| 1-methyl 1-(S)-2-methyl-1-[N-(S)-1-{[(1S,2R)-2-methoxy-4-[(S)-2-[(1R,2R)-1-methoxy-2-[N-(1R,2S)-2-hydroxy-1-methyl-2- | 2405.2 |
| phenylethylcarbamoyl]propyl]-1-pyrrolidinyl]-1-[(S)-1-methylpropyl]-4-oxobutyl]-N-methylcarbamoyl}-2- | |
| methylpropylcarbamoyl]propyl {4-[(S)-2-[(S)-3-methyl-2-[3-(2-{N-2-[4-(4-amino-1-piperidyl)-3-(6-chloro-1H-1,3- | |
| benzimidazol-2-yl)-5-(3-fluoro-5-tolyl)-2-pyridylamino]ethylcarbamoyl}ethoxy)propionylamino]butyrylamino]-5- | |
| ureidovalerylamino]-3-[(2S,3R,4S,5S,6S)-3,4,5-trihydroxy-6-{N-2-[5-(hydroxy-4,4- | |
| diphenylcyclohexyloxyphosphoryloxy)pentylcarbonylamino]ethylcarbamoyl}tetrahydro-2H-pyran-2- | |
| yloxy]phenyl}methanecarbamate | |
| 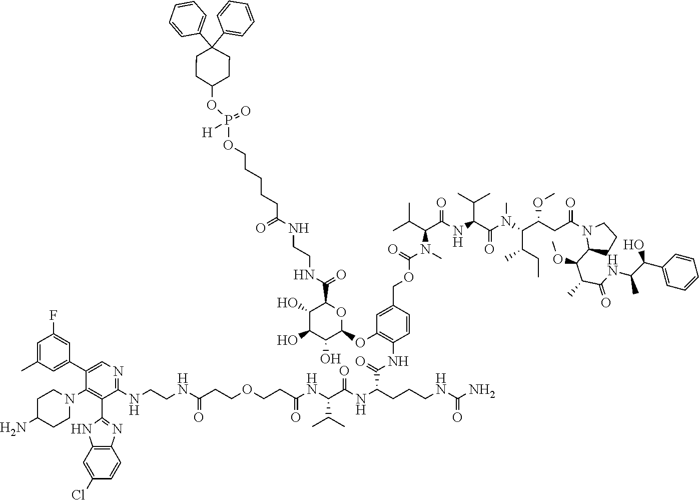 [SMILES: CC[C@H](C)[C@@H]([C@@H](CC(=O)N1CCC[C@H]1[C@H](OC)[C@@H](C)C(=O)N[C@H](C)[C@@H](O)c1ccccc1)OC)N(C)C(=O)[C@@H](NC(=O)[C@H](C(C)C)N(C)C(=O)OCc1ccc(NC(=O)[C@H](CCCNC(N)=O)NC(=O)[C@@H](NC(=O)CCOCCC(=O)NCCNc2ncc(-c3cc(C)cc(F)c3)c(N3CCC(N)CC3)c2-c2nc3ccc(Cl)cc3[nH]2)C(C)C)c(O[C@@H]2O[C@H](C(=O)NCCNC(=O)CCCCCO[PH](=O)OC3CCC(c4ccccc4)(c4ccccc4)CC3)[C@@H](O)[C@H](O)[C@H]2O)c1)C(C)C] | |
| (Compound 233) | |
| 1-methyl 1-(S)-2-methyl-1-[N-(S)-1-{[(1S,2R)-2-methoxy-4-[(S)-2-[(1R,2R)-1-methoxy-2-[N-(1R,2S)-2-hydroxy-1-methyl-2- | 2405.2 |
| phenylethylcarbamoyl]propyl]-1-pyrrolidinyl]-1-[(S)-1-methylpropyl]-4-oxobutyl]-N-methylcarbamoyl}-2- | |
| methylpropylcarbamoyl]propyl {4-[(S)-2-[(S)-3-methyl-2-[3-(2-{N-2-[4-(4-amino-1-piperidyl)-3-(6-chloro-1H-1,3- | |
| benzimidazol-2-yl)-5-(3-fluoro-5-tolyl)-2-pyridylamino]ethylcarbamoyl}ethoxy)propionylamino]butyrylamino]-5- | |
| ureidovalerylamino]-3-[(2S,3R,4S,5S,6S)-3,4,5-trihydroxy-6-({[N-6-(hydroxy-4,4- | |
| diphenylcyclohexyloxyphosphoryloxy)hexylcarbamoyl]methyl}carbamoyl)tetrahydro-2H-pyran-2- | |
| yloxy]phenyl}methanecarbamate | |
| 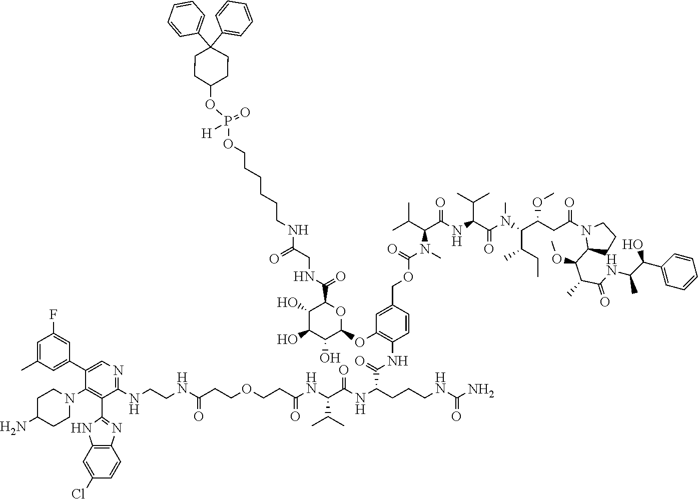 [SMILES: CC[C@H](C)[C@@H]([C@@H](CC(=O)N1CCC[C@H]1[C@H](OC)[C@@H](C)C(=O)N[C@H](C)[C@@H](O)c1ccccc1)OC)N(C)C(=O)[C@@H](NC(=O)[C@H](C(C)C)N(C)C(=O)OCc1ccc(NC(=O)[C@H](CCCNC(N)=O)NC(=O)[C@@H](NC(=O)CCOCCC(=O)NCCNc2ncc(-c3cc(C)cc(F)c3)c(N3CCC(N)CC3)c2-c2nc3ccc(Cl)cc3[nH]2)C(C)C)c(O[C@@H]2O[C@H](C(=O)NCC(=O)NCCCCCCO[PH](=O)OC3CCC(c4ccccc4)(c4ccccc4)CC3)[C@@H](O)[C@H](O)[C@H]2O)c1)C(C)C] | |
| (Compound 234) | |
| 1-{[(28,3S,4S,5R,6S)-3,4,5-trihydroxy-6-[5-({methyl[(S)-2-methyl-1-[N-(S)-1-{[(1S,2R)-2-methoxy-4-[(S)-2-[(1R,2R)-1- | 2046.0 |
| methoxy-2-[N-(1R,2S)-2-hydroxy-1-methyl-2-phenylethylcarbamoyl]propyl]-1-pyrrolidinyl]-1-[(S)-1-methylpropyl]-4- | |
| oxobutyl]-N-methylcarbamoyl}-2-methylpropylcarbamoyl]propyl](aminocarbonyloxy)}methyl)-2-[(S)-2-[(S)-3-methyl-2-[3-(2- | |
| {N-2-[4-(4-amino-1-piperidyl)-3-(6-chloro-1H-1,3-benzimidazol-2-yl)-5-(3-fluoro-5-tolyl)-2- | |
| pyridylamino]ethylcarbamoyl}ethoxy)propionylamino]butyrylamino]-5-ureidovalerylamino]phenoxy]tetrahydro-2H-pyran-2- | |
| yl]carbonyl}-4-piperidinecarboxylic acid | |
| 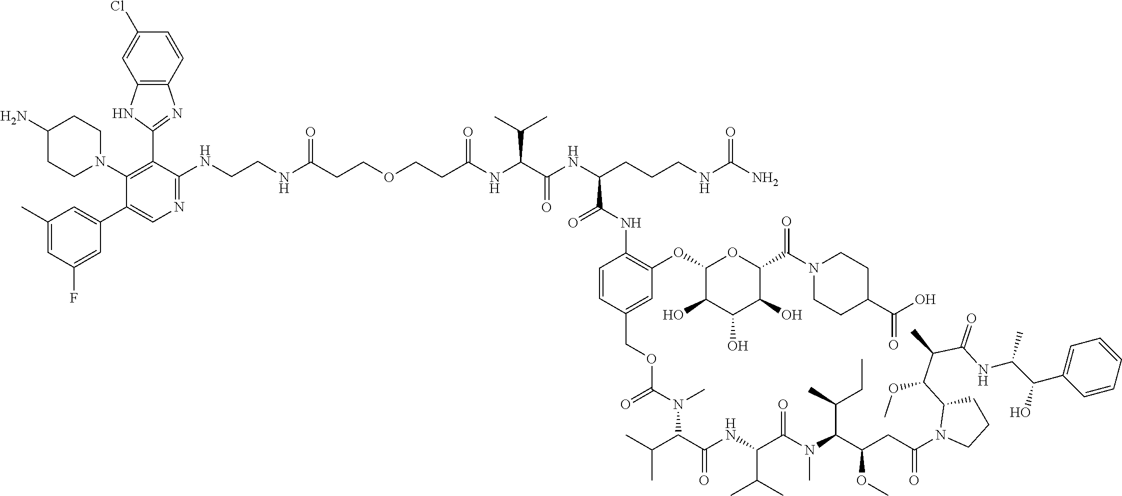 | |
| (Compound 235) | |
| (N-methyl{[N-methyl({N-methyl[(28,3S,4S,5R,6S)-3,4,5-trihydroxy-6-[5-({methyl[(S)-2-methyl-1-[N-(S)-1-{[(1S,2R)-2- | 2148.0 |
| methoxy-4-[(S)-2-[(1R,2R)-1-methoxy-2-[N-(1R,2S)-2-hydroxy-1-methyl-2-phenylethylcarbamoyl]propyl]-1-pyrrolidinyl]-1- | |
| [(S)-1-methylpropyl]-4-oxobutyl]-N-methylcarbamoyl}-2-methylpropylcarbamoyl]propyl](aminocarbonyloxy)}methyl)-2-[(S)- | |
| 2-[(S)-3-methyl-2-[3-(2-{N-2-[4-(4-amino-1-piperidyl)-3-(6-chloro-1H-1,3-benzimidazol-2-yl)-5-(3-fluoro-5-tolyl)-2- | |
| pyridylamino]ethylcarbamoyl}ethoxy)propionylamino]butyrylamino]-5-ureidovalerylamino]phenoxy]tetrahydro-2H-pyran-2- | |
| yl]carbonylamino}methyl)carbonylamino]methyl}carbonylamino)acetic acid | |
| 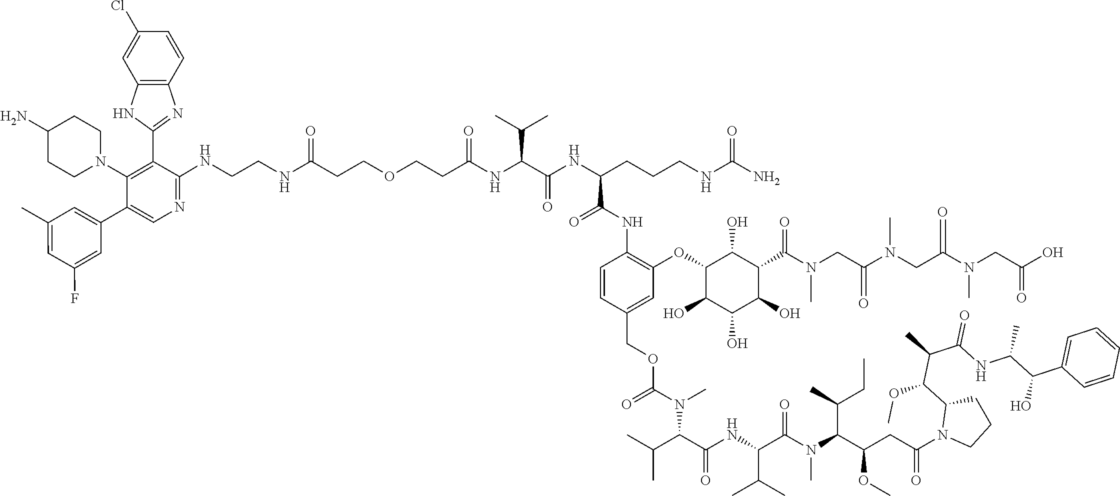 [SMILES: CC[C@H](C)[C@@H]([C@@H](CC(=O)N1CCC[C@H]1[C@H](OC)[C@@H](C)C(=O)N[C@H](C)[C@@H](O)c1ccccc1)OC)N(C)C(=O)[C@@H](NC(=O)[C@H](C(C)C)N(C)C(=O)OCc1ccc(NC(=O)[C@H](CCCNC(N)=O)NC(=O)[C@@H](NC(=O)CCOCCC(=O)NCCNc2ncc(-c3cc(C)cc(F)c3)c(N3CCC(N)CC3)c2-c2nc3ccc(Cl)cc3[nH]2)C(C)C)c(O[C@H]2[C@H](O)[C@@H](O)[C@H](O)[C@@H](C(=O)N(C)CC(=O)N(C)CC(=O)N(C)CC(=O)O)[C@H]2O)c1)C(C)C] | |
| (Compound 236) | |
| 1-methyl 1-(S)-2-methyl-1-[N-(S)-1-{[(1S,2R)-2-methoxy-4-[(S)-2-[(1R,2R)-1-methoxy-2-[N-(1R,2S)-2-hydroxy-1-methyl-2- | 2070.0 |
| phenylethylcarbamoyl]propyl]-1-pyrrolidinyl]-1-[(S)-1-methylpropyl]-4-oxobutyl]-N-methylcarbamoyl}-2- | |
| methylpropylcarbamoyl]propyl {4-[(S)-2-[(S)-3-methyl-2-{1-[2-(2-{N-2-[4-(4-amino-1-piperidyl)-3-(6-chloro-1H-1,3- | |
| benzimidazol-2-yl)-5-(3-fluoro-5-tolyl)-2-pyridylamino]ethylcarbamoyl}ethoxy)ethyl]-1H-1,2,3-triazol-4- | |
| ylcarbonylamino}butyrylamino]-5-ureidovalerylamino]-3-[(2S,3R,4S,5S,6S)-3,4,5-trihydroxy-6-[(1- | |
| piperazinyl)carbonyl]tetrahydro-2H-pyran-2-yloxy]phenyl}methanecarbamate | |
| 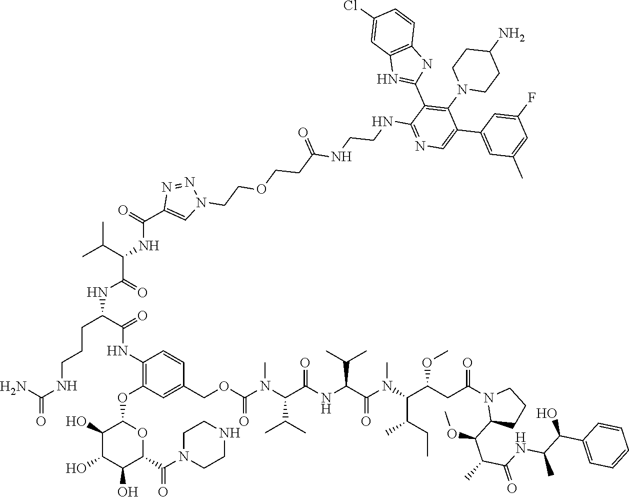 [SMILES: CC[C@H](C)[C@@H]([C@@H](CC(=O)N1CCC[C@H]1[C@H](OC)[C@@H](C)C(=O)N[C@H](C)[C@@H](O)c1ccccc1)OC)N(C)C(=O)[C@@H](NC(=O)[C@H](C(C)C)N(C)C(=O)OCc1ccc(NC(=O)[C@H](CCCNC(N)=O)NC(=O)[C@@H](NC(=O)c2cn(CCOCCC(=O)NCCNc3ncc(-c4cc(C)cc(F)c4)c(N4CCC(N)CC4)c3-c3nc4cc(Cl)ccc4[nH]3)nn2)C(C)C)c(O[C@@H]2O[C@H](C(=O)N3CCNCC3)[C@@H](O)[C@H](O)[C@H]2O)c1)C(C)C] | |
| (Compound 237) | |
| 1-methyl 1-(S)-2-methyl-1-[N-(S)-1-{[(1S,2R)-2-methoxy-4-[(S)-2-[(1R,2R)-1-methoxy-2-[N-(1R,2S)-2-hydroxy-1-methyl-2- | 2084.1 |
| phenylethylcarbamoyl]propyl]-1-pyrrolidinyl]-1-[(S)-1-methylpropyl]-4-oxobutyl]-N-methylcarbamoyl}-2- | |
| methylpropylcarbamoyl]propyl {4-[(S)-2-[(S)-3-methyl-2-[({4-[2-(2-{N-2-[4-(4-amino-1-piperidyl)-3-(6-chloro-1H-1,3- | |
| benzimidazol-2-yl)-5-(3-fluoro-5-tolyl)-2-pyridylamino]ethylcarbamoyl}ethoxy)ethyl]-1H-1,2,3-triazol-1- | |
| yl}methyl)carbonylamino]butyrylamino]-5-ureidovalerylamino]-3-[(2S,3R,4S,5S,6S)-3,4,5-trihydroxy-6-[(1- | |
| piperazinyl)carbonyl]tetrahydro-2H-pyran-2-yloxy]phenyl}methanecarbamate | |
| 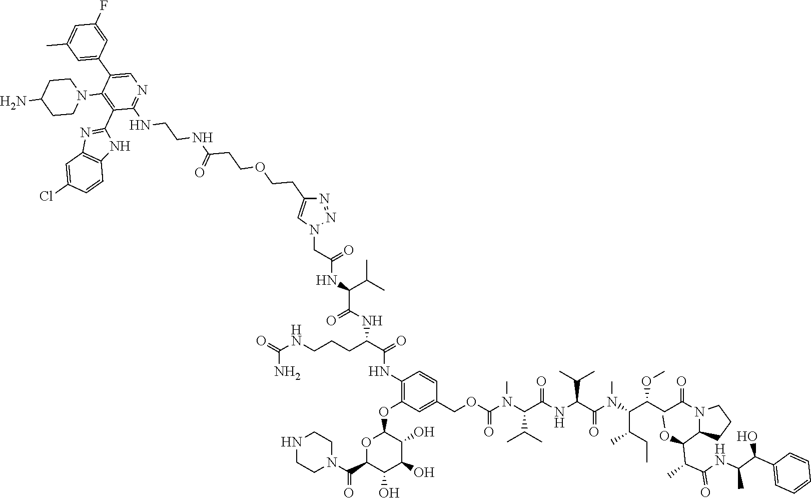 [SMILES: CC[C@H](C)[C@@H]([C@@H](CC(=O)N1CCC[C@H]1[C@H](OC)[C@@H](C)C(=O)N[C@H](C)[C@@H](O)c1ccccc1)OC)N(C)C(=O)[C@@H](NC(=O)[C@H](C(C)C)N(C)C(=O)OCc1ccc(NC(=O)[C@H](CCCNC(N)=O)NC(=O)[C@@H](NC(=O)Cn2cc(CCOCCC(=O)NCCNc3ncc(-c4cc(C)cc(F)c4)c(N4CCC(N)CC4)c3-c3nc4cc(Cl)ccc4[nH]3)nn2)C(C)C)c(O[C@@H]2O[C@H](C(=O)N3CCNCC3)[C@@H](O)[C@H](O)[C@H]2O)c1)C(C)C] | |
| (Compound 238) | |
| (S)-2,6-bis[2-({methyl[(S)-2-methyl-1-[N-(S)-1-{[(1S,2R)-2-methoxy-4-[(S)-2-[(1R,2R)-1-methoxy-2-[N-(1R,2S)-2-hydroxy- | 2201.0 |
| 1-methyl-2-phenylethylcarbamoyl]propyl]-1-pyrrolidinyl]-1-[(S)-1-methylpropyl]-4-oxobutyl]-N-methylcarbamoyl}-2- | |
| methylpropylcarbamoyl]propyl](aminocarbonyloxy)}methyl)-5-[(S)-2-[(S)-3-methyl-2-[3-(2-{N-2-[4-(4-amino-1-piperidyl)-3- | |
| (6-chloro-1H-1,3-benzimidazol-2-yl)-5-(3-fluoro-5-toly])-2- | |
| pyridylamino]ethylcarbamoyl}ethoxy)propionylamino]butyrylamino]-5-ureidovalerylamino]benzylamino]hexanoic acid | |
| 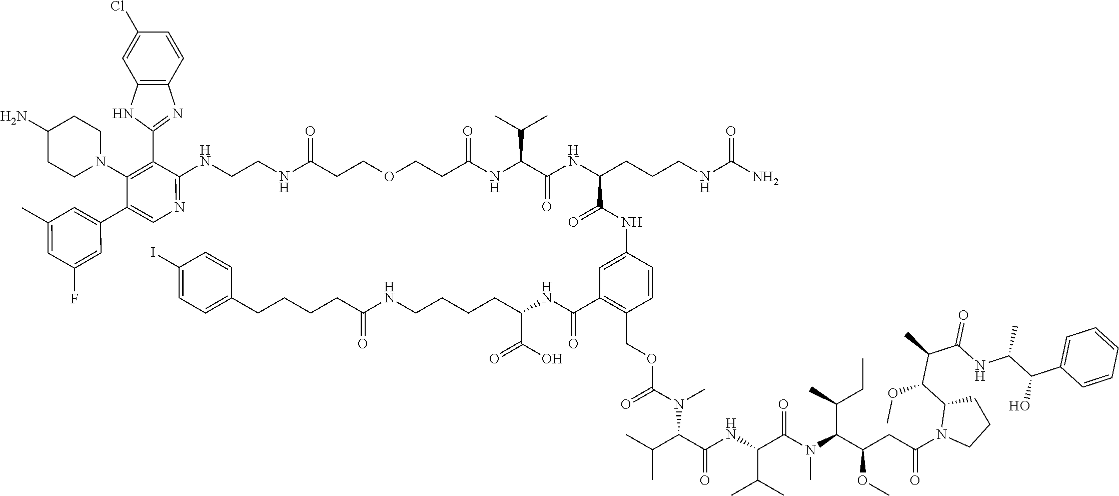 [SMILES: CC[C@H](C)[C@@H]([C@@H](CC(=O)N1CCC[C@H]1[C@H](OC)[C@@H](C)C(=O)N[C@H](C)[C@@H](O)c1ccccc1)OC)N(C)C(=O)[C@@H](NC(=O)[C@H](C(C)C)N(C)C(=O)OCc1ccc(NC(=O)[C@H](CCCNC(N)=O)NC(=O)[C@@H](NC(=O)CCOCCC(=O)NCCNc2ncc(-c3cc(C)cc(F)c3)c(N3CCC(N)CC3)c2-c2nc3ccc(Cl)cc3[nH]2)C(C)C)cc1C(=O)N[C@@H](CCCCNC(=O)CCCCc1ccc(I)cc1)C(=O)O)C(C)C] | |
| (Compound 246) | |
| (S)-2,5-bis{3-[2-({methyl[(S)-2-methyl-1-[N-(S)-1-{[(1S,2R)-2-methoxy-4-[(S)-2-[(1R,2R)-1-methoxy-2-[N-(1R,2S)-2- | 2258.0 |
| hydroxy-1-methyl-2-phenylethylcarbamoyl]propyl]-1-pyrrolidinyl]-1-[(S)-1-methylpropyl]-4-oxobutyl]-N-methylcarbamoyl}- | |
| 2-methylpropylcarbamoyl]propyl](aminocarbonyloxy)}methyl)-5-[(S)-2-[(S)-3-methyl-2-[3-(2-{N-2-[4-(4-amino-1-piperidyl)- | |
| 3-(6-chloro-1H-1,3-benzimidazol-2-yl)-5-(3-fluoro-5-tolyl)-2- | |
| pyridylamino]ethylcarbamoyl}ethoxy)propionylamino]butyrylamino]-5- | |
| ureidovalerylamino]benzylamino]propionylamino}valeric acid | |
| 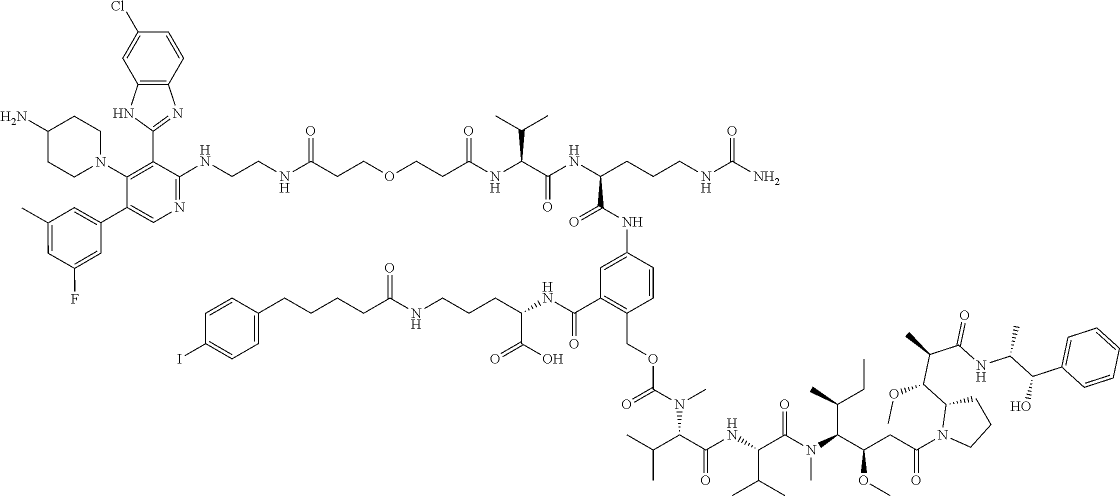 [SMILES: CC[C@H](C)[C@@H]([C@@H](CC(=O)N1CCC[C@H]1[C@H](OC)[C@@H](C)C(=O)N[C@H](C)[C@@H](O)c1ccccc1)OC)N(C)C(=O)[C@@H](NC(=O)[C@H](C(C)C)N(C)C(=O)OCc1ccc(NC(=O)[C@H](CCCNC(N)=O)NC(=O)[C@@H](NC(=O)CCOCCC(=O)NCCNc2ncc(-c3cc(C)cc(F)c3)c(N3CCC(N)CC3)c2-c2nc3ccc(Cl)cc3[nH]2)C(C)C)cc1C(=O)N[C@@H](CCCNC(=O)CCCCc1ccc(I)cc1)C(=O)O)C(C)C] | |
| (Compound 247) | |

### Example C. (S)-4-[N—(S)-1-[N—(S)-1-{N-3-[N-2-(methylamino)ethylcarbamoyl]-4-({[(S)-1-[N—(S)-1-({[(1S,2R)-4-[(S)-2-{(1R,2R)-2-[N-(1R,2S)-2-hydroxy-1-methyl-2-phenylethylcarbamoyl]-1-methoxypropyl}-1-pyrrolidinyl]-1-[(S)-1-methylpropyl]-2-methoxy-4-oxobutyl]-N-methylcarbamoyl}-2-methylpropylcarbamoyl]-2-methylpropyl](methyl)(aminocarbonyloxy)}methyl)phenylcarbamoyl}-4-ureidobutylcarbamoyl]-2-methylpropylcarbamoyl]-4-[3-(2-{N-2-[4-(4-amino-1-piperidyl)-3-(6-chloro-1H-1,3-benzimidazol-2-yl)-5-(3-fluoro-5-tolyl)-2-pyridylamino]ethylcarbamoyl}ethoxy)propionylamino]butyric acid (Compound 57)

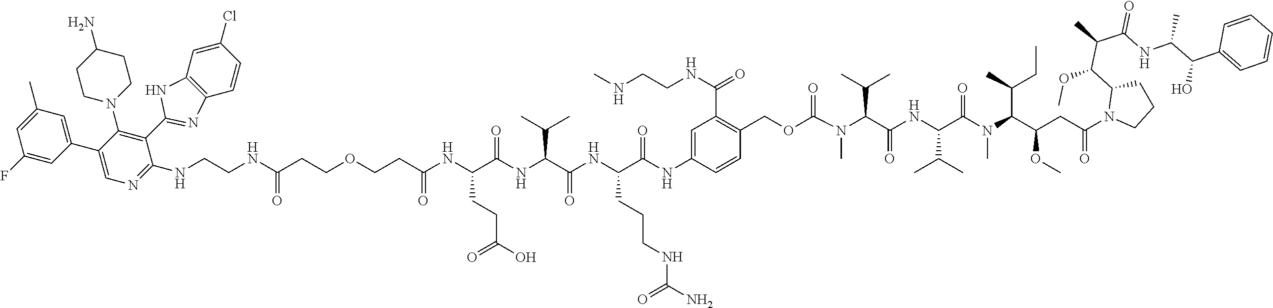 [SMILES: CC[C@H](C)[C@@H]([C@@H](CC(=O)N1CCC[C@H]1[C@H](OC)[C@@H](C)C(=O)N[C@H](C)[C@@H](O)c1ccccc1)OC)N(C)C(=O)[C@@H](NC(=O)[C@H](C(C)C)N(C)C(=O)OCc1ccc(NC(=O)[C@H](CCCNC(N)=O)NC(=O)[C@@H](NC(=O)[C@H](CCC(=O)O)NC(=O)CCOCCC(=O)NCCNc2ncc(-c3cc(C)cc(F)c3)c(N3CCC(N)CC3)c2-c2nc3ccc(Cl)cc3[nH]2)C(C)C)cc1C(=O)NCCNC)C(C)C]

Step C-1, preparation of tert-butyl (2-(5-amino-2-(hydroxymethyl)benzamido)ethyl)(methyl)carbamate: Into a 40-mL vial was placed 6-aminoisobenzofuran-1(3H)-one (1.5 g, 1 Eq, 10 mmol), tert-butyl (2-aminoethyl)(methyl)carbamate (8.0 g, 4.6 Eq, 46 mmol), and methanol (15 mL). The reaction mixture was stirred at 35° C. for 24 hours. The mixture was concentrated under reduced pressure. The crude product was purified by MPLC with the following conditions: Silica gel column 80 g, PE/EtOAc system, the ratio of EtOAc from 0% to 75% in 25 min, Flow rate: 50 mL/min; Wave Length: 254 nm. The collected fractions were concentrated under reduced pressure to provide tert-butyl (2-(5-amino-2-(hydroxymethyl)benzamido)ethyl)(methyl)carbamate (2.0 g, 6.2 mmol, 61%).

Step C-2, preparation of tert-butyl (2-(5-((S)-2-((S)-2-((((9H-fluoren-9-yl)methoxy)carbonyl)amino)-3-methylbutanamido)-5-ureidopentanamido)-2-(hydroxymethyl)benzamido)ethyl)(methyl)carbamate: Into a 40-mL vial was placed tert-butyl (2-(5-amino-2-(hydroxymethyl)benzamido)ethyl)(methyl)carbamate (1.0 g, 1 Eq, 3.1 mmol), EEDQ (1 g, 1 Eq, 4 mmol) and (S)-2-((S)-2-((((9H-fluoren-9-yl)methoxy)carbonyl)amino)-3-methylbutanamido)-5-ureidopentanoic acid (1.6 g, 1.0 Eq, 3.2 mmol). The reaction mixture was stirred at 21° C. for 16 hours. The mixture was concentrated under reduced pressure. The crude product was purified by MPLC with the following conditions: Silica gel column 120 g, PE/EtOAc system, the ratio of EtOAc from 0% to 100% in 15 min, Flow rate: 80 mL/min; Wave Length: 254 nm, DCM-MeOH system, the ratio of MeOH from 0% to 10% in 20 min, Flowrate: 80 mL/min; Wave Length: 254 nm. The collected fractions were concentrated under reduced pressure to provide tert-butyl (2-(5-((S)-2-((S)-2-((((9H-fluoren-9-yl)methoxy)carbonyl)amino)-3-methylbutanamido)-5-ureidopentanamido)-2-(hydroxymethyl)benzamido)ethyl)(methyl)carbamate (1.25 g, 1.56 mmol, 50%); (M+H)⁺=802.3.

Step C-3, preparation of tert-butyl (2-(5-((S)-2-((S)-2-((((9H-fluoren-9-yl)methoxy)carbonyl)amino)-3-methylbutanamido)-5-ureidopentanamido)-2-((((4-nitrophenoxy)carbonyl)oxy)methyl)benamido)ethyl)(methyl)carbamate: Into a 40-mL vial was placed with a mixture of tert-butyl (2-(5-((S)-2-((S)-2-((((9H-fluoren-9-yl)methoxy)carbonyl)amino)-3-methylbutanamido)-5-ureidopentanamido)-2-(hydroxymethyl)benzamido)ethyl)(methyl)carbamate (600 mg, 1 Eq, 748 μmol), bis(4-nitrophenyl) carbonate (685 mg, 3.01 Eq, 2.25 mmol), N-ethyl-N-isopropylpropan-2-amine (290 mg, 3.00 Eq, 2.24 mmol), and dimethylformamide (6 mL), dimethylaminopyridine (DMAP) (50 mg, 0.55 Eq, 0.41 mmol). The reaction mixture was stirred at 22° C. for 3 hours. The crude product was purified by Prep-HPLC with the following conditions: Column, WelFlash™, C18 120 g, Spherical 20-40 μm; Mobile phase, Water (0.1% formic acid) and ACN (5% ACN to 5% ACN in 1 min, 30% ACN up to 90% in 6 min, 95% ACN to 95% in 3 min); Total flow rate, 70 mL/min; Detector, UV 220 nm. The collected fractions were dried by lyophilization to provide tert-butyl (2-(5-((S)-2-((S)-2-((((9H-fluoren-9-yl)methoxy)carbonyl)amino)-3-methylbutanamido)-5-ureidopentanamido)-2-((((4-nitrophenoxy)carbonyl)oxy)methyl)benzamido)ethyl)(methyl)carbamate (230 mg, 238 μmol, 31.8%); (M+H)⁺=967.7.

Step C-4, preparation of 4-((S)-2-((S)-2-((((9H-fluoren-9-yl)methoxy)carbonyl)amino)-3-methylbutanamido)-5-ureidopentanamido)-2-((2-((tert-butoxycarbonyl)(methyl)amino)ethyl)carbamoyl)benzyl ((S)-1-(((S)-1-(((3R,4S,5S)-1-((S)-2-((1R,2R)-3-(((1S,2R)-1-hydroxy-1-Phenylpropan-2-yl)amino)-1-methoxy-2-methyl-3-oxopropyl)pyrrolidin-1-yl)-3-methoxy-5-methyl-1-oxoheptan-4-yl)(methyl)amino)-3-methyl-1-oxobutan-2-yl)amino)-3-methyl-1-oxobutan-2-yl)(methyl)carbamate: Into a 8-mL vial was placed a mixture of tert-butyl (2-(5-((S)-2-((S)-2-((((9H-fluoren-9-yl)methoxy)carbonyl)amino)-3-methylbutanamido)-5-ureidopentanamido)-2-((((4-nitrophenoxy)carbonyl)oxy)methyl)benzamido)ethyl)(methyl)carbamate (210 mg, 1 Eq, 217 μmol), 1H-benzo[d][1,2,3]triazol-1-ol hydrate (40 mg, 1.2 Eq, 0.26 mmol), N-ethyl-N-isopropylpropan-2-amine (85 mg, 3.0 Eq, 0.66 mmol), (S)—N-((3R,4S,5S)-1-((S)-2-((1R,2R)-3-(((1S,2R)-1-hydroxy-1-phenylpropan-2-yl)amino)-1-methoxy-2-methyl-3-oxopropyl)pyrrolidin-1-yl)-3-methoxy-5-methyl-1-oxoheptan-4-yl)-N,3-dimethyl-2-((S)-3-methyl-2-(methylamino)butanamido)butanamide (125 mg, 0.802 Eq, 174 μmol) and dimethylformamide (DMF) (2 mL). The reaction mixture was stirred at 21° C. for 2 hours. The mixture was directly purified by MPLC with the following conditions: Column, WelFlash™, C18 120 g, Spherical 20-40 μm; Mobile phase, Water (0.1% formic acid) and ACN (5% ACN to 5% ACN in 1 min, 30% ACN up to 98% in 6 min, 98% ACN to 98% in 3 min); Total flowrate, 70 mL % min; Detector, UV 220 nm. The collected fractions were concentrated to provide 4-((S)-2-((S)-2-((((9H-fluoren-9-yl)methoxy)carbonyl)amino)-3-methylbutanamido)-5-ureidopentanamido)-2-((2-((tert-butoxycarbonyl)(methyl)amino)ethyl)carbamoyl)benzyl ((S)-1-(((S)-1-(((3R,4S,5S)-1-((S)-2-((1R,2R)-3-(((1S,2R)-1-hydroxy-1-phenylpropan-2-yl)amino)-1-methoxy-2-methyl-3-oxopropyl)pyrrolidin-1-yl)-3-methoxy-5-methyl-1-oxoheptan-4-yl)(methyl)amino)-3-methyl-1-oxobutan-2-yl)amino)-3-methyl-1-oxobutan-2-yl)(methyl)carbamate (145 mg, 93.8 μmol, 43.2%); (M/2+H)⁺=773.7.

Step C-5, preparation of 4-((S)-2-((S)-2-amino-3-methylbutanamido)-5-ureidopentanamido)-2-((2-((tert-butoxycarbonyl)(methyl)amino)ethyl)carbamoyl)benzyl ((S)-1-(((S)-1-(((3R,4S,5S)-1-((S)-2-((1R,2R)-3-(((1S,2R)-1-hydroxy-1-phenylpropan-2-yl)amino)-1-methoxy-2-methyl-3-oxopropyl)pyrrolidin-1-yl)-3-methoxy-5-methyl-1-oxoheptan-4-yl)(methyl)amino)-3-methyl-1-oxobutan-2-yl)amino)-3-methyl-1-oxobutan-2-yl)(methyl)carbamate: Into a 8-mL vial was placed a mixture of 4-((S)-2-((S)-2-((((9H-fluoren-9-yl)methoxy)carbonyl)amino)-3-methylbutanamido)-5-ureidopentanamido)-2-((2-((tert-butoxycarbonyl)methyl)amino)ethyl)carbamoyl)benzyl ((S)-1-(((S)-1-(((3R,4S,5S)-1-((S)-2-((1R,2R)-3-(((1S,2R)-1-hydroxy-1-phenylpropan-2-yl)amino)-1-methoxy-2-methyl-3-oxopropyl)pyrrolidin-1-yl)-3-methoxy-5-methyl-1-oxoheptan-4-yl)(methyl)amino)-3-methyl-1-oxobutan-2-yl)amino)-3-methyl-1-oxobutan-2-yl)(methyl)carbamate (145 mg, 1 Eq, 93.8 μmol), DBU (50 mg, 50 μL, 3.5 Eq, 0.33 mmol) and ACN (1.5 mL). The reaction mixture was stirred at 23° C. for 1 hour. The crude product was purified by Prep-HPLC with the following conditions: Column: SunFire prep OBD 19*150 mm 5 um; Mobile Phase A: Water (0.1% trifluoroacetic acid); Mobile Phase B: ACN; Gradient: 23% B to 50% B in 10 min; Flow rate: 20 mL/min; Wave Length: 220 nm. The collected fractions were dried by lyophilization to provide 4-((S)-2-((S)-2-amino-3-methylbutanamido)-5-ureidopentanamido)-2-((2-((tert-butoxy carbonyl)methyl)amino)ethyl)carbamoyl)benzyl ((S)-1-(((S)-1-(((3R,4S,5S)-1-((S)-2-((1R,2R)-3-(((1S,2R)-1-hydroxy-1-phenylpropan-2-yl)amino)-1-methoxy-2-methyl-3-oxopropyl)pyrrolidin-1-yl)-3-methoxy-5-methyl-1-oxoheptan-4-yl)(methyl)amino)-3-methyl-1-oxobutan-2-yl)amino)-3-methyl-1-oxobutan-2-yl)methyl)carbamate (130 mg, 88 μmol, 94%, 90% Purity); (M+H)⁺=1323.0.

Step C-6, preparation of tert-butyl (S)-4-((((9H-fluoren-9-yl)methoxy)carbonyl)amino)-5-(((S)-1-(((S)-1-((3-((2-((tert-butoxycarbonyl)(methyl)amino)ethyl)carbamoyl)-4-((5S,8S,11S,12R)-11-((S)-sec-butyl)-12-(2-((S)-2-((1R,2R)-3-((1S,2R)-1-hydroxy-1-phenylpropan-2-yl)amino)-1-methoxy-2-methyl-3-oxopropyl)pyrrolidin-1-yl)-2-oxoethyl)-5,8-diisopropyl-4,10-dimethyl-3,6,9-trioxo-2,13-dioxa-4,7,10-triazatetradecyl)phenyl)amino)-1-oxo-5-ureidopentan-2-yl)amino)-3-methyl-1-oxobutan-2-yl)amino)-5-oxopentanoate: Into a 8-mL vial was placed a mixture of (S)-2-((((9H-fluoren-9-yl)methoxy)carbonyl)amino)-5-(tert-butoxy)-5-oxopentanoic acid (25 mg, 1.2 Eq, 59 μmol), HATU (25 mg, 1.3 Eq, 66 μmol), diisopropylethylamine (DIEA) (20 mg, 27 μL, 3.2 Eq, 0.15 mmol) and dimethylformamide (DMF) (1 mL). The reaction mixture was stirred at 22° C. for 15 minutes. Then 4-((S)-2-((S)-2-amino-3-methylbutanamido)-5-ureidopentanamido)-2-((2-((tert-butoxycarbonyl)(methyl)amino)ethyl)carbamoyl)benzyl ((S)-1-(((S)-1-(((3R,4S,5S)-1-((S)-2-((1R,2R)-3-(((1S,2R)-1-hydroxy-1-phenylpropan-2-yl)amino)-1-methoxy-2-methyl-3-oxopropyl)pyrrolidin-1-yl)-3-methoxy-5-methyl-1-oxoheptan-4-yl)(methyl)amino)-3-methyl-1-oxobutan-2-yl)amino)-3-methyl-1-oxobutan-2-yl)(methyl)carbamate (65 mg, 1 Eq, 49 μmol) was added. The reaction mixture was stirred at 22° C. for 1 hour. There was 63% product in LCMS, which was used directly for next step without any purification. (M+H)⁺=1730.9.

Step C-7, preparation of tert-butyl (S)-4-amino-5-(((S)-1-(((S)-1-((3-((2-((tert-butoxycarbonyl)(methyl)amino)ethyl)carbamoyl)-4-((5S,8S,11S,12R)-11-((S)-sec-butyl)-12-(2-((S)-2-((1R,2R)-3-(((1S,2R)-1-hydroxy-1-phenylpropan-2-yl)amino)-1-methoxy-2-methyl-3-oxopropyl)pyrrolidin-1-yl)-2-oxoethyl)-5,8-diisopropyl-4,10-dimethyl-3,6,9-trioxo-2,13-dioxa-4,7,10-triazatetradecyl)phenyl)amino)-1-oxo-5-ureidopentan-2-yl)amino)-3-methyl-1-oxobutan-2-yl)amino)-5-oxopentanoate: Into a 8-mL vial was placed a mixture of tert-butyl (S)-4-((((9H-fluoren-9-yl)methoxy)carbonyl)amino)-5-(((S)-1-(((S)-1-((3-((2-((tert-butoxycarbonyl)(methyl)amino)ethyl)carbamoyl)-4-((5S,8S,11S,12R)-11-((S)-sec-butyl)-12-(2-((S)-2-((1R,2R)-3-(((1S,2R)-1-hydroxy-1-phenylpropan-2-yl)amino)-1-methoxy-2-methyl-3-oxopropyl)pyrrolidin-1-yl)-2-oxoethyl)-5,8-diisopropyl-4,10-dimethyl-3,6,9-trioxo-2,13-dioxa-4,7,10-triazatetradecyl)phenyl)amino)-1-oxo-5-ureidopentan-2-yl)amino)-3-methyl-1-oxobutan-2-yl)amino)-5-oxopentanoate (60 mg, 1 Eq, 35 μmol), DBU (20 mg, 20 μL, 3.8 Eq, 0.13 mmol) and DMF (0.5 mL). The reaction mixture was stirred at 21° C. for 1 hour. The crude product was purified by Prep-HPLC with the following conditions: Column: SunFire prep OBD 19*150 mm5 um; Mobile Phase A: Water (0.1% trifluoracetic acid (TFA)): Mobile Phase B: ACN; Gradient: 25% B to 50% B in 10 min: Flow rate: 20 mL/min: Wave Length: 220 nm. The collected fractions were dried by lyophilization to provide tert-butyl (S)-4-amino-5-(((S)-1-(((S)-1-((3-((2-((tert-butoxycarbonyl)(methyl)amino)ethyl)carbamoyl)-4-((5S,8S,11S,12R)-11-((S)-sec-butyl)-12-(2-((S)-2-((1R,2R)-3-(((1S,2R)-1-hydroxy-1-phenylpropan-2-yl)amino)-1-methoxy-2-methyl-3-oxopropyl)pyrrolidin-1-yl)-2-oxoethyl)-5,8-diisopropyl-4,10-dimethyl-3,6,9-trioxo-2,13-dioxa-4,7,10-triazatetradecyl)phenyl)amino)-1-oxo-5-ureidopentan-2-yl)amino)-3-methyl-1-oxobutan-2-yl)amino)-5-oxopentanoate (40 mg, 27 μmol, 76%); (M+H+Na)⁺=1530.8.

Step C-8, preparation of tert-butyl (S)-5-(((S)-1-(((S)-1-((3-((2-((tert-butoxycarbonyl)(methyl)amino)ethyl)carbamoyl)-4-((5S,8S,11S,12R)-11-((S)-sec-butyl)-12-(2-((S)-2-((1R,2R)-3-(((1S,2R)-1-hydroxy-1-phenylpropan-2-yl)amino)-1-methoxy-2-methyl-3-oxopropyl)pyrrolidin-1-yl)-2-oxoethyl)-5,8-diisopropyl-4,10-dimethyl-3,6,9-trioxo-2,13-dioxa-4,7,10-triazatetradecyl)phenyl)amino)-1-oxo-5-ureidopentan-2-yl)amino)-3-methyl-1-oxobutan-2-yl)amino)-4-(3-(3-((2-((4-(4-((tert-butoxycarbonyl)amino)piperidin-1-yl)-3-(6-chloro-1H-benzo[d]imidazol-2-yl)-5-(3-fluoro-5-methylphenyl)pyridin-2-yl)amino)ethyl)amino)-3-oxopropoxy)propanamido)-5-oxopentanoate: Into a 8-mL vial was placed a mixture of 3-(3-((2-((4-(4-((tert-butoxycarbonyl)amino)piperidin-1-yl)-3-(6-chloro-1H-benzo[d]imidazol-2-yl)-5-(3-fluoro-5-methylphenyl)pyridin-2-yl)amino)ethyl)amino)-3-oxopropoxy)propanoic acid (20 mg, 1.2 Eq, 27 μmol), perfluorophenyldiphenylphosphinate (12 mg, 1.3 Eq, 31 μmol), 4-methylmorpholine (10 mg, 4.3 Eq. 99 μmol), and DMF (0.5 mL). The reaction mixture was stirred at 22° C. for 15 minutes. Then tert-butyl (S)-4-amino-5-(((S)-1-(((S)-1-((3-((2-((tert-butoxycarbonyl)(methyl)amino)ethyl)carbamoyl)-4-((5S,8S,11S,12R)-11-((S)-sec-butyl)-12-(2-((S)-2-((1R,2R)-3-(((1S,2R)-1-hydroxy-1-phenylpropan-2-yl)amino)-1-methoxy-2-methyl-3-oxopropyl)pyrrolidin-1-yl)-2-oxoethyl)-5,8-diisopropyl-4,10-dimethyl-3,6,9-trioxo-2,13-dioxa-4,7,10-triazatetradecyl)phenyl)amino)-1-oxo-5-ureidopentan-2-yl)amino)-3-methyl-1-oxobutan-2-yl)amino)-5-oxopentanoate (35 mg, 1 Eq, 23 μmol) was added. The reaction mixture was stirred at 22° C. for 1 hour. The crude product was purified by Prep-HPLC with the following conditions: Column: SunFire prep OBD 19*150 mm 5 um; Mobile Phase A: Water (0.1% TFA); Mobile Phase B: ACN; Gradient: 30% B to 61% B in 10 min; Flowrate: 20 mL/min; Wave Length: 220 nm. The collected fractions were dried by lyophilization to provide tert-butyl (S)-5-(((S)-1-(((S)-1-((3-((2-((tert-butoxycarbonyl)(methyl)amino)ethyl)carbamoyl)-4-((5S,8S,11S,12R)-11-((S)-sec-butyl)-12-(2-((S)-2-((1R,2R)-3-(((1S,2R)-1-hydroxy-1-phenylpropan-2-yl)amino)-1-methoxy-2-methyl-3-oxopropyl)pyrrolidin-1-yl)-2-oxoethyl)-5,8-diisopropyl-4,10-dimethyl-3,6,9-trioxo-2,13-dioxa-4,7,10-triazatetradecyl)phenyl)amino)-1-oxo-5-ureidopentan-2-yl)amino)-3-methyl-1-oxobutan-2-yl)amino)-4-(3-(3-((2-((4-(4-((tert-butoxy carbonyl)amino)piperidin-1-yl)-3-(6-chloro-1H-benzo[d]imidazol-2-yl)-5-(3-fluoro-5-methylphenyl)pyridin-2-yl)amino)ethyl)amino)-3-oxopropoxy)propanamido)-5-oxopentanoate (30 mg, 13 μmol, 58%); (M/2+H)⁺=1115.5, 1116.5.

Step C-9, preparation of (S)-4-[N—(S)-1-[N—(S)-1-{N-3-[N-2-(methylamino)ethylcarbamoyl]-4-({[(S)-1-[N—(S)-1-{[(1S,2R)-4-[(S)-2-{(1R,2R)-2-[N-(1R,2S)-2-hydroxy-1-methyl-2-phenylethylcarbamoyl]-1-methoxypropyl}-1-pyrrolidinyl]-1-[(S)-1-methylpropyl]-2-methoxy-4-oxobutyl-N-methylcarbamoyl}-2-methylpropylcarbamoyl]-2-methylpropyl](methyl)(aminocarbonyloxy)}methyl)phenylcarbamoy}-4-ureidobutylcarbamoyl]-2-methylpropylcarbamoyl]-4-[3-(2-{N-2-[4-(4-amino-1-piperidyl)-3-(6-chloro-1H-1,3-benzimidazol-2-yl)-5-(3-fluoro-5-tolyl)-2-pyridylamino]ethylcarbamoyl}ethoxy)propionylamino]butyric acid: Into a 8-mL vial was placed a mixture of tert-butyl (S)-5-(((S)-1-(((S)-1-((3-((2-((tert-butoxycarbonyl)(methyl)amino)ethyl)carbamoyl)-4-((5S,8S,11S,12R)-11-((S)-sec-butyl)-12-(2-((S)-2-((1R,2R)-3-(((1S,2R)-1-hydroxy-1-phenylpropan-2-yl)amino)-1-methoxy-2-methyl-3-oxopropyl)pyrrolidin-1-yl)-2-oxoethyl)-5,8-diisopropyl-4,10-dimethyl-3,6,9-trioxo-2,13-dioxa-4,7,10-triazatetradecyl)phenyl)amino)-1-oxo-5-ureidopentan-2-yl)amino)-3-methyl-1-oxobutan-2-yl)amino)-4-(3-(3-((2-((4-(4-((tert-butoxy carbonyl)amino)piperidin-1-yl)-3-(6-chloro-1H-benzo[d]imidazol-2-yl)-5-(3-fluoro-5-methylphenyl)pyridin-2-yl)amino)ethyl)amino)-3-oxopropoxy)propanamido)-5-oxopentanoate (30 mg, 1 Eq, 13 μmol), DCM (0.3 mL) and TFA (0.15 mL). The reaction mixture was stirred at 0° C. for 4 hours. DIEA was added to the reaction solution to adjust the pH to basic, and blown with nitrogen until dry. The crude product was purified by Prep-HPLC with the following conditions: Column: SunFire prep OBD 19*150 mm 5 um; Mobile Phase A: Water (0.1% TFA): Mobile Phase B: ACN: Gradient: 28% B to 69% B in 10 min; Flow rate: 20 mL/min; Wave Length: 220 nm. The collected fractions were dried by lyophilization to provide ((S)-4-[N—(S)-1-[N—(S)-1-{N-3-[N-2-(methylamino)ethylcarbamoyl]-4-({[(S)-1-[N—(S)-1-{[(1S,2R)-4-[(S)-2-{(1R,2R)-2-[N-(1R,2S)-2-hydroxy-1-methyl-2-phenylethylcarbamoyl]-1-methoxypropyl}-1-pyrrolidinyl]-1-[(S)-1-methylpropyl]-2-methoxy-4-oxobutyl]-N-methylcarbamoyl}-2-methylpropylcarbamoyl]-2-methylpropyl](methyl)(aminocarbonyloxy)}methyl)phenylcarbamoyl}-4-ureidobutylcarbamoyl]-2-methylpropylcarbamoyl]-4-[3-(2-{N-2-[4-(4-amino-1-piperidyl)-3-(6-chloro-1H-1,3-benzimidazol-2-yl)-5-(3-fluoro-5-tolyl)-2-pyridylamino]ethylcarbamoyl}ethoxy)propionylamino]butyric acid (7.3 mg, 3.2 μmol, 23%); (M+H)⁺=1972.0, 1974.0.

The following conjugates were prepared similarly to Example C with appropriate substituting reagents and substrates at different steps and they may require additional functional group modifications via well-known chemistry with appropriate reagents.

| | MS |
|---|---|
| Compound | (M+H)⁺ |
| [(2-{3-[N-(S)-1-[N-(S)-1-[N-3-(N-2-aminoethylcarbamoyl)-4-({[(S)-1-[N-(S)-1-{[(1S,2R)-4-[(S)-2-{(1R,2R)-2-[N-(1R,2S)-2- | 2122.1 |
| hydroxy-1-methyl-2-phenylethylcarbamoyl]-1-methoxypropyl}-1-pyrrolidinyl]-[(S)-1-methylpropyl]-2-methoxy-4-oxobutyl]-N- | |
| methylcarbamoyl}-2-methylpropylcarbamoyl]-2-methylpropyl](methyl)(aminocarbonyloxy)}methyl)phenylcarbamoyl]-4- | |
| ureidobutylcarbamoyl]-2-methylpropylcarbamoyl]-5-[2-(2-{N-2-[4-(4-amino-1-piperidyl)-3-(6-chloro-1H-1,3-benzimidazol-2-yl)-5- | |
| (3-fluoro-5-tolyl)-2-pyridylamino]ethylcarbamoyl}ethoxy)ethylamino]benzylamino}ethyl)(carboxymethyl)amino]acetic acid | |
| 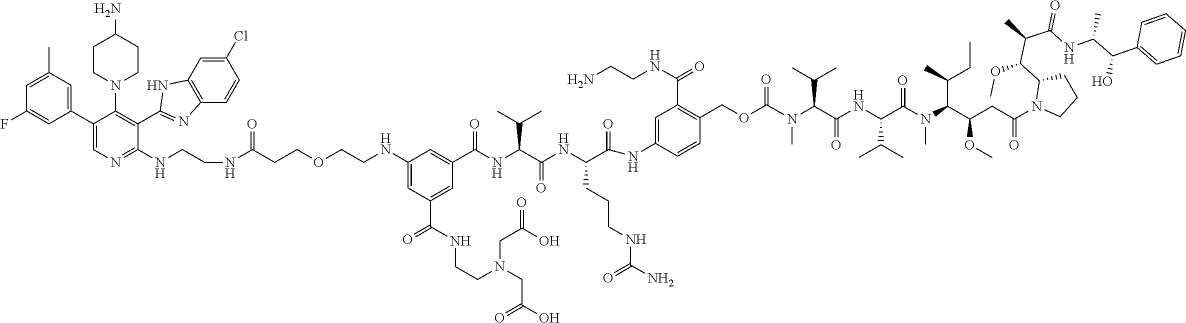 [SMILES: CC[C@H](C)[C@@H]([C@@H](CC(=O)N1CCC[C@H]1[C@H](OC)[C@@H](C)C(=O)N[C@H](C)[C@@H](O)c1ccccc1)OC)N(C)C(=O)[C@@H](NC(=O)[C@H](C(C)C)N(C)C(=O)OCc1ccc(NC(=O)[C@H](CCCNC(N)=O)NC(=O)[C@@H](NC(=O)c2cc(NCCOCCC(=O)NCCNc3ncc(-c4cc(C)cc(F)c4)c(N4CCC(N)CC4)c3-c3nc4ccc(Cl)cc4[nH]3)cc(C(=O)NCCN(CC(=O)O)CC(=O)O)c2)C(C)C)cc1C(=O)NCCN)C(C)C] | |
| (Compound 58) | |
| 1-(S)-1-[N-(S)-1-{[(1S,2R)-4-[(S)-2-{(1R,2R)-2-[N-(1R,2S)-2-hydroxy-1-methyl-2-phenylethylcarbamoyl]-1-methoxypropyl}-1- | 1973.1 |
| pyrrolidinyl]-1-[(S)-1-methylpropyl]-2-methoxy-4-oxobutyl]-N-methylcarbamoyl}-2-methylpropylcarbamoyl]-2-methylpropyl 1- | |
| methyl{4-[(S)-2-[(S)-2-(14-{N-2-[4-(4-amino-1-piperidyl)-3-(6-chloro-1H-1,3-benzimidazol-2-yl)-5-(3-fluoro-5-toly])-2- | |
| pyridylamino]ethylcarbamoyl}-3,6,9,12-tetraoxatetradecylcarbonylamino)-3-methylbutyrylamino]-5-ureidovalerylamino]-2-[(3-amino- | |
| 1-azetidinyl)carbonyl\|phenyl}methanecarbamate | |
| 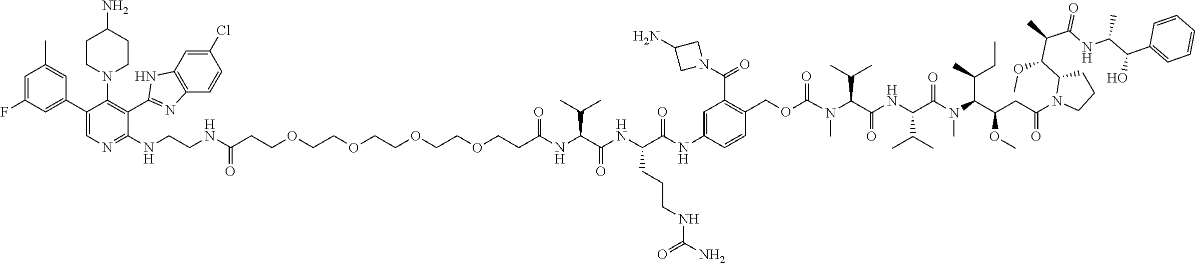 [SMILES: CC[C@H](C)[C@@H]([C@@H](CC(=O)N1CCC[C@H]1[C@H](OC)[C@@H](C)C(=O)N[C@H](C)[C@@H](O)c1ccccc1)OC)N(C)C(=O)[C@@H](NC(=O)[C@H](C(C)C)N(C)C(=O)OCc1ccc(NC(=O)[C@H](CCCNC(N)=O)NC(=O)[C@@H](NC(=O)CCOCCOCCOCCOCCC(=O)NCCNc2ncc(-c3cc(C)cc(F)c3)c(N3CCC(N)CC3)c2-c2nc3ccc(Cl)cc3[nH]2)C(C)C)cc1C(=O)N1CC(N)C1)C(C)C] | |
| (Compound 59) | |
| [2-({[(S)-1-[N-(S)-1-{[(1S,2R)-4-[(S)-2-{(1R,2R)-2-[N-(1R,2S)-2-hydroxy-1-methyl-2-phenylethylcarbamoyl]-1-methoxypropyl}-1- | 1843.9 |
| pyrrolidinyl]-1-[(S)-1-methylpropyl]-2-methoxy-4-oxobutyl]-N-methylcarbamoyl}-2-methylpropylcarbamoyl]-2- | |
| methylpropyl](methyl)(aminocarbonyloxy)}methyl)-5-[(S)-2-[(S)-2-[3-(2-{N-2-[4-(4-amino-1-piperidyl)-3-(6-chloro-1H-1,3- | |
| benzimidazol-2-yl)-5-(3-fluoro-5-tolyl)-2-pyridylamino]ethylcarbamoyl}ethoxy)propionylamino]-3-methylbutyrylamino]-5- | |
| ureidovalerylamino]benzylamino]acetic acid | |
| 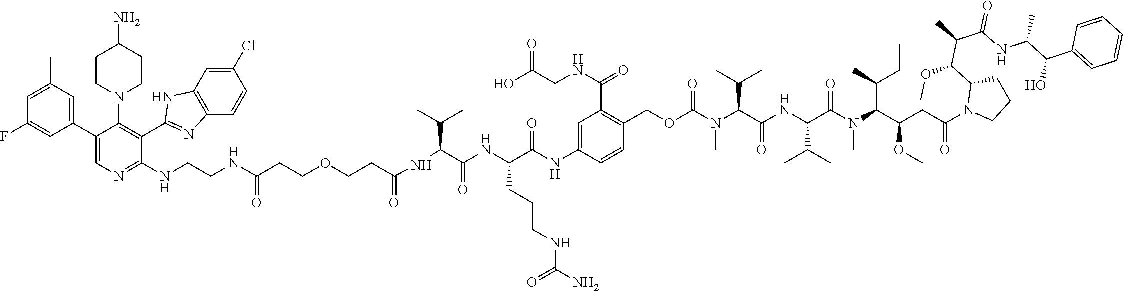 [SMILES: CC[C@H](C)[C@@H]([C@@H](CC(=O)N1CCC[C@H]1[C@H](OC)[C@@H](C)C(=O)N[C@H](C)[C@@H](O)c1ccccc1)OC)N(C)C(=O)[C@@H](NC(=O)[C@H](C(C)C)N(C)C(=O)OCc1ccc(NC(=O)[C@H](CCCNC(N)=O)NC(=O)[C@@H](NC(=O)CCOCCC(=O)NCCNc2ncc(-c3cc(C)cc(F)c3)c(N3CCC(N)CC3)c2-c2nc3ccc(Cl)cc3[nH]2)C(C)C)cc1C(=O)NCC(=O)O)C(C)C] | |
| (Compound 60) | |
| 1-(S)-1-[N-(S)-1-{[(1S,2R)-4-[(S)-2-{(1R,2R)-2-[N-(1R,2S)-2-hydroxy-1-methyl-2-phenylethylcarbamoyl]-1-methoxypropyl}-1- | 2101.1 |
| pyrrolidinyl]-1-[(S)-1-methylpropyl]-2-methoxy-4-oxobutyl]-N-methylcarbamoyl}-2-methylpropylcarbamoyl]-2-methylpropyl 1 - | |
| methyl {4-[(S)-2-[(S)-2-[(S)-2-(14-{N-2-[4-(4-amino-1-piperidyl)-3-(6-chloro-1H-1,3-benzimidazol-2-yl)-5-(3-fluoro-5-tolyl)-2- | |
| pyridylamino]ethylcarbamoyl}-3,6,9,12-tetraoxatetradecylcarbonylamino)-4-carbamoylbutyrylamino]-3-methylbutyrylamino]-5- | |
| ureidovalerylamino]-2-[(3-amino-1-azetidinyl)carbonyl]phenyl}methanecarbamate | |
| 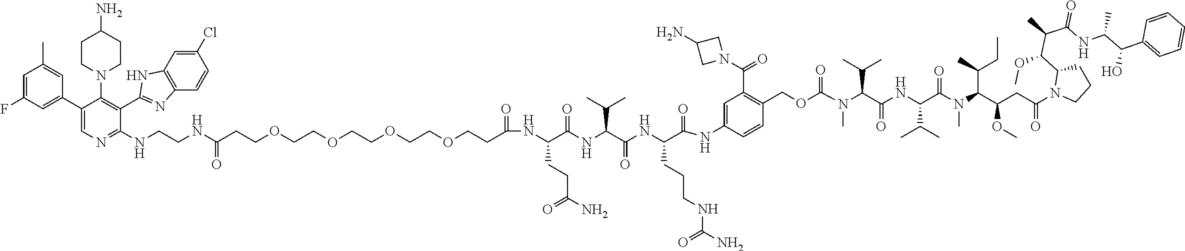 [SMILES: CC[C@H](C)[C@@H]([C@@H](CC(=O)N1CCC[C@H]1[C@H](OC)[C@@H](C)C(=O)N[C@H](C)[C@@H](O)c1ccccc1)OC)N(C)C(=O)[C@@H](NC(=O)[C@H](C(C)C)N(C)C(=O)OCc1ccc(NC(=O)[C@H](CCCNC(N)=O)NC(=O)[C@@H](NC(=O)[C@H](CCC(N)=O)NC(=O)CCOCCOCCOCCOCCC(=O)NCCNc2ncc(-c3cc(C)cc(F)c3)c(N3CCC(N)CC3)c2-c2nc3ccc(Cl)cc3[nH]2)C(C)C)cc1C(=O)N1CC(N)C1)C(C)C] | |
| (Compound 61) | |
| 2-[2-({[(S)-1-[N-(S)-1-{[(1S,2R)-4-[(S)-2-{(1R,2R)-2-[N-(1R,2S)-2-hydroxy-1-methyl-2-phenylethylcarbamoyl]]-1-methoxypropyl}-1- | 2026.0 |
| pyrrolidinyl]-1-[(S)-1-methylpropyl]-2-methoxy-4-oxobutyl]-N-methylcarbamoyl}-2-methylpropylcarbamoyl]-2- | |
| methylpropyl](methyl)(aminocarbonyloxy)}methyl)-5-[(S)-2-[(S)-2-(14-{N-2-[4-(4-amino-1-piperidyl)-3-(6-chloro-1H-1,3- | |
| benzimidazol-2-yl)-5-(3-fluoro-5-tolyl)-2-pyridylamino]ethylcarbamoyl}-3,6,9,12-tetraoxatetradecylcarbonylamino)-3- | |
| methylbutyrylamino]-5-ureidovalerylamino]benzylamino]ethanesulfonic acid | |
| 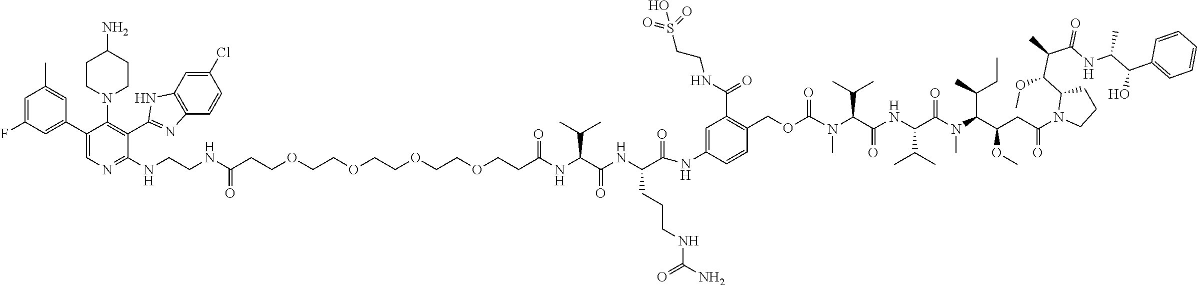 [SMILES: CC[C@H](C)[C@@H]([C@@H](CC(=O)N1CCC[C@H]1[C@H](OC)[C@@H](C)C(=O)N[C@H](C)[C@@H](O)c1ccccc1)OC)N(C)C(=O)[C@@H](NC(=O)[C@H](C(C)C)N(C)C(=O)OCc1ccc(NC(=O)[C@H](CCCNC(N)=O)NC(=O)[C@@H](NC(=O)CCOCCOCCOCCOCCC(=O)NCCNc2ncc(-c3cc(C)cc(F)c3)c(N3CCC(N)CC3)c2-c2nc3ccc(Cl)cc3[nH]2)C(C)C)cc1C(=O)NCCS(=O)(=O)O)C(C)C] | |
| (Compound 62) | |
| (S)-4-[N-(S)-1-[N-(S)-1-{N-3-[N-2-(dimethylamino)ethylcarbamoyl]-4-({[(S)-1-[N-(S)-1-{[(1S,2R)-4-[(S)-2-{(1R,2R)-2-[N-(1R,2S)- | 1986.0 |
| 2-hydroxy-1-methyl-2-phenylethylcarbamoyl]-1-methoxypropyl}-1-pyrrolidinyl]-1-[(S)-1-methylpropyl]-2-methoxy-4-oxobutyl]-N- | |
| methylcarbamoyl}-2-methylpropylcarbamoyl]-2-methylpropyl](methyl)(aminocarbonyloxy)}methyl)phenylcarbamoyl}-4- | |
| ureidobutylcarbamoyl]-2-methylpropylcarbamoyl]-4-[3-(2-{N-2-[4-(4-amino-1-piperidyl)-3-(6-chloro-1H-1,3-benzimidazol-2-yl)-5- | |
| (3-fluoro-5-tolyl)-2-pyridylamino]ethylcarbamoyl}ethoxy)propionylamino]butyric acid | |
| 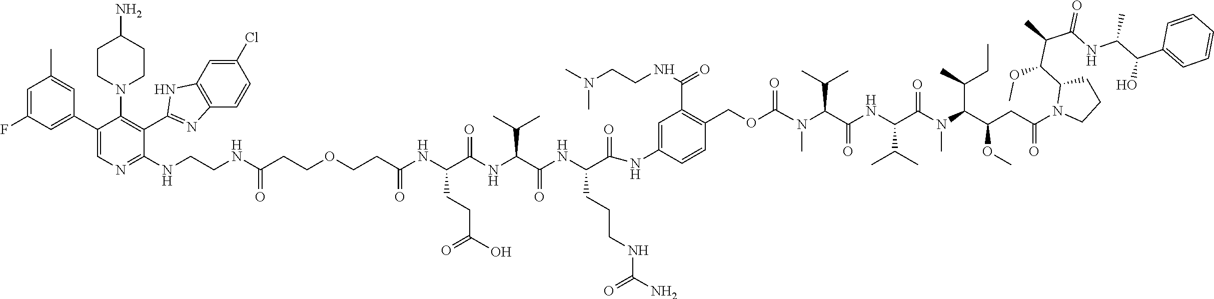 [SMILES: CC[C@H](C)[C@@H]([C@@H](CC(=O)N1CCC[C@H]1[C@H](OC)[C@@H](C)C(=O)N[C@H](C)[C@@H](O)c1ccccc1)OC)N(C)C(=O)[C@@H](NC(=O)[C@H](C(C)C)N(C)C(=O)OCc1ccc(NC(=O)[C@H](CCCNC(N)=O)NC(=O)[C@@H](NC(=O)[C@H](CCC(=O)O)NC(=O)CCOCCC(=O)NCCNc2ncc(-c3cc(C)cc(F)c3)c(N3CCC(N)CC3)c2-c2nc3ccc(Cl)cc3[nH]2)C(C)C)cc1C(=O)NCCN(C)C)C(C)C] | |
| (Compound 64) | |
| (S)-4-[N-(S)-1-[N-(S)-1-[N-4-({[(S)-1-[N-(S)-1-{[(1S,2R)-4-[(S)-2-{(1R,2R)-2-[N-(1R,2S)-2-hydroxy-1-methyl-2- | 2102.1 |
| phenylethylcarbamoyl]-1-methoxypropyl}-1-pyrrolidinyl]-1-[(S)-1-methylpropyl]-2-methoxy-4-oxobutyl]-N-methylcarbamoyl}-2- | |
| methylpropylcarbamoyl]-2-methylpropyl](methyl)(aminocarbonyloxy)}methyl)-3-[(3-amino-1-azetidinyl)carbonyl]phenylcarbamoyl]- | |
| 4-ureidobutylcarbamoyl]-2-methylpropylcarbamoyl]-4-(14-{N-2-[4-(4-amino-1-piperidyl)-3-(6-chloro-1H-1,3-benzimidazol-2-yl)-5- | |
| (3-fluoro-5-tolyl)-2-pyridylamino]ethylcarbamoyl}-3,6,9,12-tetraoxatetradecylcarbonylamino)butyric acid | |
| 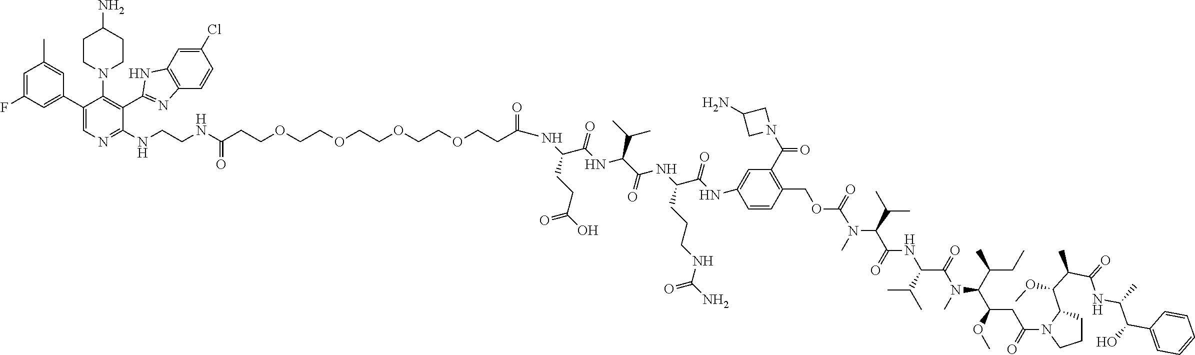 [SMILES: CC[C@H](C)[C@@H]([C@@H](CC(=O)N1CCC[C@H]1[C@H](OC)[C@@H](C)C(=O)N[C@H](C)[C@@H](O)c1ccccc1)OC)N(C)C(=O)[C@@H](NC(=O)[C@H](C(C)C)N(C)C(=O)OCc1ccc(NC(=O)[C@H](CCCNC(N)=O)NC(=O)[C@@H](NC(=O)[C@H](CCC(=O)O)NC(=O)CCOCCOCCOCCOCCC(=O)NCCNc2ncc(-c3cc(C)cc(F)c3)c(N3CCC(N)CC3)c2-c2nc3ccc(Cl)cc3[nH]2)C(C)C)cc1C(=O)N1CC(N)C1)C(C)C] | |
| (Compound 65) | |
| 1-(S)-1-[N-(S)-1-{[(1S,2R)-4-[(S)-2-{(1R,2R)-2-[N-(1R,2S)-2-hydroxy-1-methyl-2-phenylethylcarbamoyl]-1-methoxypropyl}-1- | 1896.0 |
| pyrrolidinyl]-1-[(S)-1-methylpropyl]-2-methoxy-4-oxobutyl]-N-methylcarbamoyl}-2-methylpropylcarbamoyl]-2-methylpropyl 1- | |
| methyl [2-(N-2-aminoethylcarbamoyl)-4-[(S)-2-[(S)-2-[(S)-2-[3-(2-{N-2-[4-(4-amino-1-piperidyl)-3-(6-chloro-1H-1,3-benzimidazol-2- | |
| yl)-5-(3-fluoro-5-tolyl)-2-pyridylamino]ethylcarbamoyl}ethoxy)propionylamino]-4-(1H-tetraazol-5-yl)butyrylamino]-3- | |
| methylbutyrylamino]propionylamino]phenyl]methanecarbamate | |
| 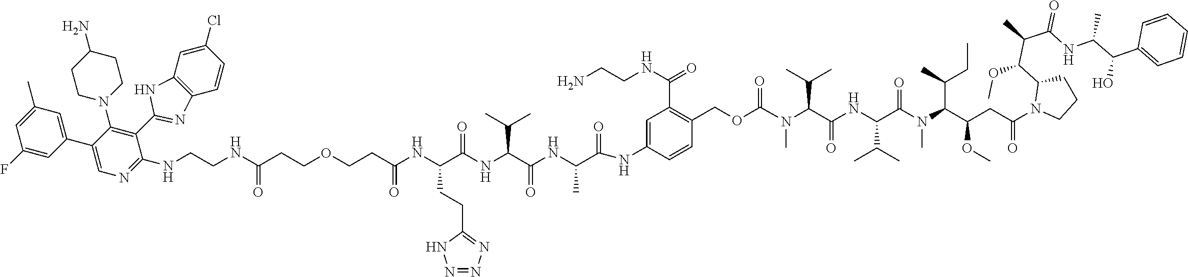 [SMILES: CC[C@H](C)[C@@H]([C@@H](CC(=O)N1CCC[C@H]1[C@H](OC)[C@@H](C)C(=O)N[C@H](C)[C@@H](O)c1ccccc1)OC)N(C)C(=O)[C@@H](NC(=O)[C@H](C(C)C)N(C)C(=O)OCc1ccc(NC(=O)[C@H](C)NC(=O)[C@@H](NC(=O)[C@H](CCc2nnn[nH]2)NC(=O)CCOCCC(=O)NCCNc2ncc(-c3cc(C)cc(F)c3)c(N3CCC(N)CC3)c2-c2nc3ccc(Cl)cc3[nH]2)C(C)C)cc1C(=O)NCCN)C(C)C] | |
| (Compound 66) | |
| 1-[2-({[(S)-1-[N-(S)-1-{[(1S,2R)-4-[(S)-2-{(1R,2R)-2-[N-(1R,2S)-2-hydroxy-1-methyl-2-phenylethylcarbamoyl]-1-methoxypropyl}-1- | 2002.0 |
| pyrrolidinyl]-1-[(S)-1-methylpropyl]-2-methoxy-4-oxobutyl]-N-methylcarbamoyl}-2-methylpropylcarbamoyl]-2- | |
| methylpropyl](methyl)(aminocarbonyloxy)}methyl)-5-[(S)-2-[(S)-2-(14-{N-2-[4-(4-amino-1-piperidyl)-3-(6-chloro-1H-1,3- | |
| benzimidazol-2-yl)-5-(3-fluoro-5-tolyl)-2-pyridylamino]ethylcarbamoyl}-3,6,9,12-tetraoxatetradecylcarbonylamino)-3- | |
| methylbutyrylamino]-5-ureidovalerylamino]benzoyl]-3-azetidinecarboxylic acid | |
| 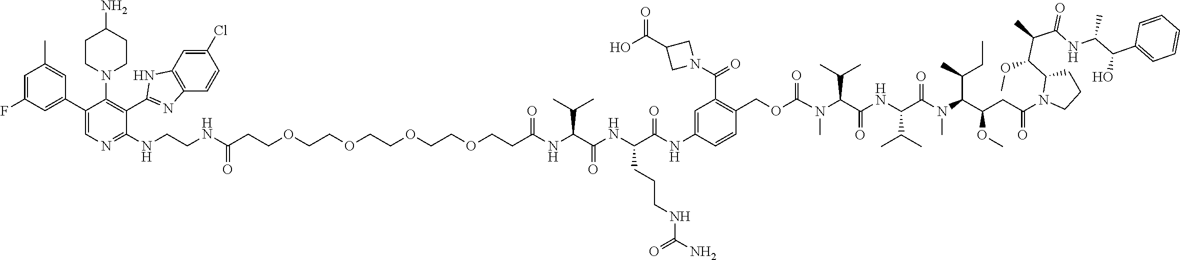 [SMILES: CC[C@H](C)[C@@H]([C@@H](CC(=O)N1CCC[C@H]1[C@H](OC)[C@@H](C)C(=O)N[C@H](C)[C@@H](O)c1ccccc1)OC)N(C)C(=O)[C@@H](NC(=O)[C@H](C(C)C)N(C)C(=O)OCc1ccc(NC(=O)[C@H](CCCNC(N)=O)NC(=O)[C@@H](NC(=O)CCOCCOCCOCCOCCC(=O)NCCNc2ncc(-c3cc(C)cc(F)c3)c(N3CCC(N)CC3)c2-c2nc3ccc(Cl)cc3[nH]2)C(C)C)cc1C(=O)N1CC(C(=O)O)C1)C(C)C] | |
| (Compound 67) | |
| [2-({[(S)-1-[N-(S)-1-{[(1S,2R)-4-[(S)-2-{(1R,2R)-2-[N-(1R,2S)-2-hydroxy-1-methyl-2-phenylethylcarbamoyl]-1-methoxypropyl}-1- | 1890.0 |
| pyrrolidinyl]-1-[(S)-1-methylpropyl]-2-methoxy-4-oxobutyl]-N-methylcarbamoyl}-2-methylpropylcarbamoyl]-2- | |
| methylpropyl](methyl)(aminocarbonyloxy)}methyl)-5-[(S)-2-[(S)-2-(14-{N-2-[4-(4-amino-1-piperidyl)-3-(6-chloro-1H-1,3- | |
| benzimidazol-2-yl)-5-(3-fluoro-5-tolyl)-2-pyridylamino]ethylcarbamoyl}-3,6,9,12-tetraoxatetradecylcarbonylamino)-3- | |
| methylbutyrylamino]propionylamino]benzylamino]acetic acid | |
| 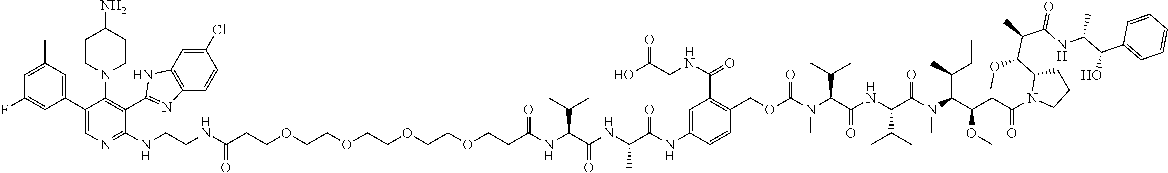 [SMILES: CC[C@H](C)[C@@H]([C@@H](CC(=O)N1CCC[C@H]1[C@H](OC)[C@@H](C)C(=O)N[C@H](C)[C@@H](O)c1ccccc1)OC)N(C)C(=O)[C@@H](NC(=O)[C@H](C(C)C)N(C)C(=O)OCc1ccc(NC(=O)[C@H](C)NC(=O)[C@@H](NC(=O)CCOCCOCCOCCOCCC(=O)NCCNc2ncc(-c3cc(C)cc(F)c3)c(N3CCC(N)CC3)c2-c2nc3ccc(Cl)cc3[nH]2)C(C)C)cc1C(=O)NCC(=O)O)C(C)C] | |
| (Compound 69) | |
| [2-({[(S)-1-[N-(S)-1-{[(1S,2R)-4-[(S)-2-{(1R,2R)-2-[N-(1R,2S)-2-hydroxy-1-methyl-2-phenylethylcarbamoyl]-1-methoxypropyl}-1- | 1915.0 |
| pyrrolidinyl]-1-[(S)-1-methylpropyl]-2-methoxy-4-oxobutyl]-N-methylcarbamoyl}-2-methylpropylcarbamoyl]-2- | |
| methylpropyl](methyl)(aminocarbonyloxy)} methyl)-5-[(S)-2-[(S)-2-[({N-methyl]2-(2-{N-2-[4-(4-amino-1-piperidyl)-3-(6-chloro-1H- | |
| 1,3-benzimidazol-2-yl)-5-(3-fluoro-5-tolyl)-2-pyridylamino]ethylcarbamoyl}ethoxy)ethyl]carbonylamino}methyl)carbonylamino]-3- | |
| methylbutyrylamino]-5-ureidovalerylamino]benzylamino]acetic acid | |
| 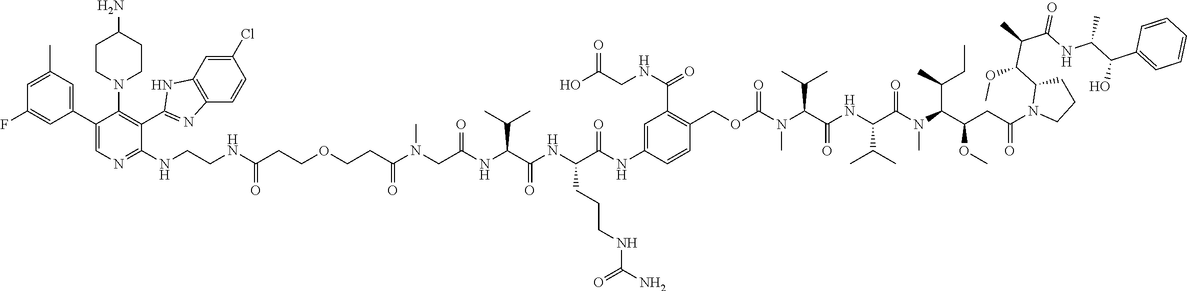 [SMILES: CC[C@H](C)[C@@H]([C@@H](CC(=O)N1CCC[C@H]1[C@H](OC)[C@@H](C)C(=O)N[C@H](C)[C@@H](O)c1ccccc1)OC)N(C)C(=O)[C@@H](NC(=O)[C@H](C(C)C)N(C)C(=O)OCc1ccc(NC(=O)[C@H](CCCNC(N)=O)NC(=O)[C@@H](NC(=O)CN(C)C(=O)CCOCCC(=O)NCCNc2ncc(-c3cc(C)cc(F)c3)c(N3CCC(N)CC3)c2-c2nc3ccc(Cl)cc3[nH]2)C(C)C)cc1C(=O)NCC(=O)O)C(C)C] | |
| (Compound 71) | |
| [2-({[(S)-1-[N-(S)-1-{[(1S,2R)-4-[(S)-2-{(1R,2R)-2-[N-(1R,2S)-2-hydroxy-1-methyl-2-phenylethylcarbamoyl]-1-methoxypropyl}-1- | 1915.0 |
| pyrrolidinyl]-1-[(S)-1-methylpropyl]-2-methoxy-4-oxobutyl]-N-methylcarbamoyl}-2-methylpropylcarbamoyl]-2- | |
| methylpropyl](methyl)(aminocarbonyloxy)}methyl)-5-[(S)-2-[(S)-2-(3-{2-[({N-2-[4-(4-amino-1-piperidyl)-3-(6-chloro-1H-1,3- | |
| benzimidazol-2-yl)-5-(3-fluoro-5-tolyl)-2-pyridylamino]ethylcarbamoyl}methyl)-N-methylcarbamoyl]ethoxy}propionylamino)-3- | |
| methylbutyrylamino]-5-ureidovalerylamino]benzylamino]acetic acid | |
| 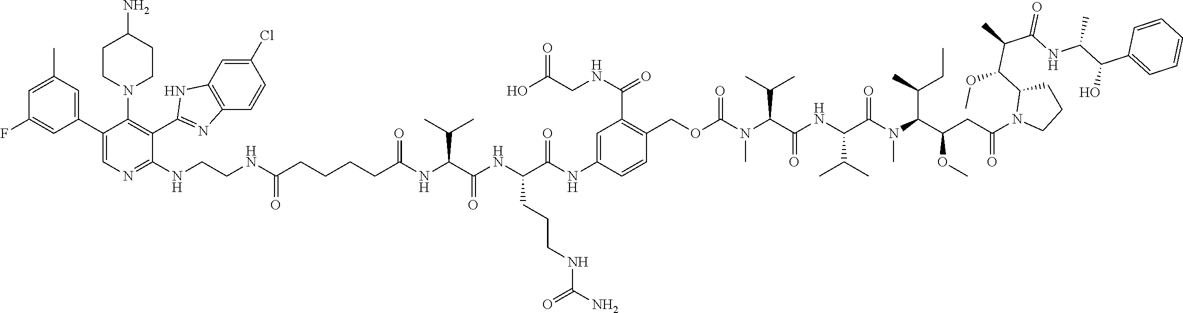 [SMILES: CC[C@H](C)[C@@H]([C@@H](CC(=O)N1CCC[C@H]1[C@H](OC)[C@@H](C)C(=O)N[C@H](C)[C@@H](O)c1ccccc1)OC)N(C)C(=O)[C@@H](NC(=O)[C@H](C(C)C)N(C)C(=O)OCc1ccc(NC(=O)[C@H](CCCNC(N)=O)NC(=O)[C@@H](NC(=O)CCCCC(=O)NCCNc2ncc(-c3cc(C)cc(F)c3)c(N3CCC(N)CC3)c2-c2nc3ccc(Cl)cc3[nH]2)C(C)C)cc1C(=O)NCC(=O)O)C(C)C] | |
| (Compound 72) | |
| 1-[2-({[(S)-1-[N-(S)-1-{[(1S,2R)-4-[(S)-2-{(1R,2R)-2-[N-(1R,2S)-2-hydroxy-1-methyl-2-phenylethylcarbamoyl]-1-methoxypropyl}-1- | 1956.0 |
| pyrrolidinyl]-1-[(S)-1-methylpropyl]-2-methoxy-4-oxobutyl]-N-methylcarbamoyl}-2-methylpropylcarbamoyl]-2- | |
| methylpropyl](methyl)(aminocarbonyloxy)}methyl)-5-[(S)-2-[(S)-2-[(R)-3-[N-2-(2-{N-2-[4-(4-amino-1-piperidyl)-3-(6-chloro-1H- | |
| 1,3-benzimidazol-2-yl)-5-(3-fluoro-5-tolyl)-2-pyridylamino]ethylcarbamoyl}ethoxy)ethylcarbamoyl]-3-aminopropionylamino]-3- | |
| methylbutyrylamino]-5-ureidovalerylamino]benzoyl]-3-azetidinecarboxylic acid | |
| 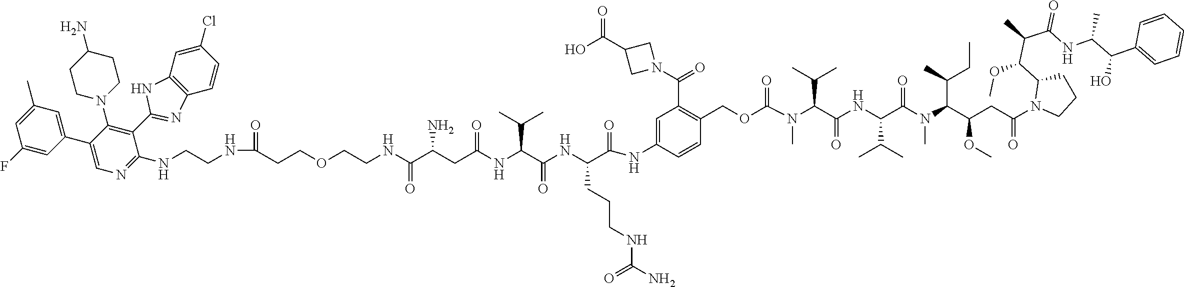 [SMILES: CC[C@H](C)[C@@H]([C@@H](CC(=O)N1CCC[C@H]1[C@H](OC)[C@@H](C)C(=O)N[C@H](C)[C@@H](O)c1ccccc1)OC)N(C)C(=O)[C@@H](NC(=O)[C@H](C(C)C)N(C)C(=O)OCc1ccc(NC(=O)[C@H](CCCNC(N)=O)NC(=O)[C@@H](NC(=O)C[C@@H](N)C(=O)NCCOCCC(=O)NCCNc2ncc(-c3cc(C)cc(F)c3)c(N3CCC(N)CC3)c2-c2nc3ccc(Cl)cc3[nH]2)C(C)C)cc1C(=O)N1CC(C(=O)O)C1)C(C)C] | |
| (Compound 73) | |
| 15-{N-2-[2-({[(S)-1-[N-(S)-1-{[(1S,2R)-4-[(S)-2-{(1R,2R)-2-[N-(1R,2S)-2-hydroxy-1-methyl-2-phenylethylcarbamoyl]-1- | 2149.1 |
| methoxypropyl}-1-pyrrolidinyl]-1-[(S)-1-methylpropyl]-2-methoxy-4-oxobutyl]-N-methylcarbamoyl}-2-methylpropylcarbamoyl]-2- | |
| methylpropyl](methyl)(aminocarbonyloxy)methyl)-5-[(S)-2-[(S)-2-{3-[2-(2-{N-2-[4-(4-amino-1-piperidyl)-3-(6-chloro-1H-1,3- | |
| benzimidazol-2-yl)-5-(3-fluoro-5-tolyl)-2-pyridylamino]ethylcarbamoyl}ethoxy)ethoxy]propionylamino}-3-methylbutyrylamino]-5- | |
| ureidovalerylamino]benzylamino]ethylcarbamoyl}-4,7,10,13-tetraoxapentadecanoic acid | |
| 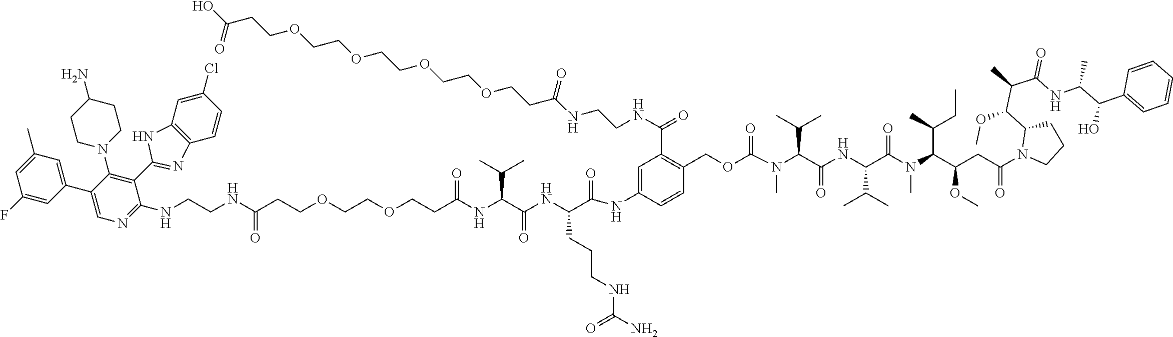 [SMILES: CC[C@H](C)[C@@H]([C@@H](CC(=O)N1CCC[C@H]1[C@H](OC)[C@@H](C)C(=O)N[C@H](C)[C@@H](O)c1ccccc1)OC)N(C)C(=O)[C@@H](NC(=O)[C@H](C(C)C)N(C)C(=O)OCc1ccc(NC(=O)[C@H](CCCNC(N)=O)NC(=O)[C@@H](NC(=O)CCOCCOCCC(=O)NCCNc2ncc(-c3cc(C)cc(F)c3)c(N3CCC(N)CC3)c2-c2nc3ccc(Cl)cc3[nH]2)C(C)C)cc1C(=O)NCCNC(=O)CCOCCOCCOCCOCCC(=O)O)C(C)C] | |
| (Compound 74) | |
| 11-[2-({[(S)-1-[N-(S)-1-{[(18,2R)-4-[(S)-2-{(1R,2R)-2-[N-(1R,2S)-2-hydroxy-1-methyl-2-phenylethylcarbamoyl]-1-methoxypropyl}- | 2014.1 |
| 1-pyrrolidinyl]-1-[(S)-1-methylpropyl]-2-methoxy-4-oxobutyl]-N-methylcarbamoyl}-2-methylpropylcarbamoyl]-2- | |
| methylpropyl](methyl)(aminocarbonyloxy)}methyl)-5-[(S)-2-[(S)-2-{3-[2-(2-{N-2-[4-(4-amino-1-piperidyl)-3-(6-chloro-1H-1,3- | |
| benzimidazol-2-yl)-5-(3-fluoro-5-tolyl)-2-pyridylamino]ethylcarbamoyl}ethoxy)ethoxy]propionylamino}-3-methylbutyrylamino]-5- | |
| ureidovalerylamino]benzylamino]undecanoic acid | |
| 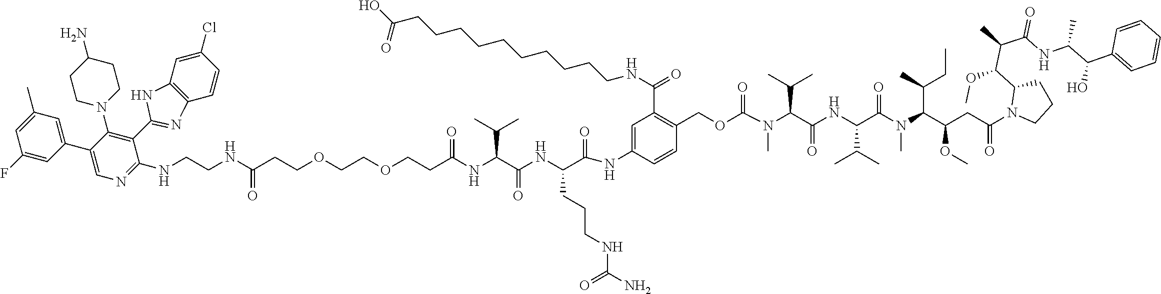 [SMILES: CC[C@H](C)[C@@H]([C@@H](CC(=O)N1CCC[C@H]1[C@H](OC)[C@@H](C)C(=O)N[C@H](C)[C@@H](O)c1ccccc1)OC)N(C)C(=O)[C@@H](NC(=O)[C@H](C(C)C)N(C)C(=O)OCc1ccc(NC(=O)[C@H](CCCNC(N)=O)NC(=O)[C@@H](NC(=O)CCOCCOCCC(=O)NCCNc2ncc(-c3cc(C)cc(F)c3)c(N3CCC(N)CC3)c2-c2nc3ccc(Cl)cc3[nH]2)C(C)C)cc1C(=O)NCCCCCCCCCCC(=O)O)C(C)C] | |
| (Compound 75) | |
| (S)-2-[N-(S)-1-[N-(S)-1-[N-4-({[(S)-1-[N-(S)-1-{[(1S,2R)-4-[(S)-2-{(1R,2R)-2-[N-(1R,2S)-2-hydroxy-1-methyl-2- | 1992.0 |
| phenylethylcarbamoyl]-1-methoxypropyl}-1-pyrrolidinyl]-1-[(S)-1-methylpropyl]-2-methoxy-4-oxobutyl]-N-methylcarbamoyl}-2- | |
| methylpropylcarbamoyl]-2-methylpropyl](methyl)(aminocarbonyloxy)}methyl)-3-[(3-amino-1-azetidinyl)carbonyl]phenylcarbamoyl]- | |
| 4-ureidobutylcarbamoyl]-2-methylpropylcarbamoyl]-2-[3-(2-{N-2-[4-(4-amino-1-piperidyl)-3-(6-chloro-1H-1,3-benzimidazol-2-yl)-5- | |
| (3-fluoro-5-tolyl)-2-pyridylamino]ethylcarbamoyl}ethoxy)propionylamino]ethanesulfonic acid | |
| 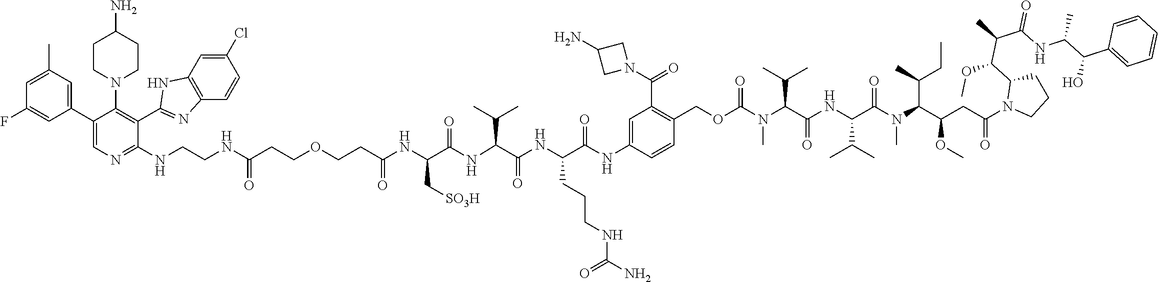 [SMILES: CC[C@H](C)[C@@H]([C@@H](CC(=O)N1CCC[C@H]1[C@H](OC)[C@@H](C)C(=O)N[C@H](C)[C@@H](O)c1ccccc1)OC)N(C)C(=O)[C@@H](NC(=O)[C@H](C(C)C)N(C)C(=O)OCc1ccc(NC(=O)[C@H](CCCNC(N)=O)NC(=O)[C@@H](NC(=O)[C@@H](CS(=O)(=O)O)NC(=O)CCOCCC(=O)NCCNc2ncc(-c3cc(C)cc(F)c3)c(N3CCC(N)CC3)c2-c2nc3ccc(Cl)cc3[nH]2)C(C)C)cc1C(=O)N1CC(N)C1)C(C)C] | |
| (Compound 76) | |
| 1-[2-({[(S)-1-[N-(S)-1-{[(1S,2R)-4-[(S)-2-{(1R,2R)-2-[N-(1R,2S)-2-hydroxy-1-methyl-2-phenylethylcarbamoyl]-1-methoxypropyl}-1- | 2020.9 |
| pyrrolidinyl]-1-[(S)-1-methylpropyl]-2-methoxy-4-oxobutyl]-N-methylcarbamoyl}-2-methylpropylcarbamoyl]-2- | |
| methylpropyl](methyl)(aminocarbonyloxy)} methyl)-5-[(S)-2-[(S)-2-[(S)-2-[3-(2-{N-2-[4-(4-amino-1-piperidyl)-3-(6-chloro-1H-1,3- | |
| benzimidazol-2-yl)-5-(3-fluoro-5-tolyl)-2-pyridylamino]ethylcarbamoyl}ethoxy)propionylamino]-3-sulfopropionylamino]-3- | |
| methylbutyrylamino]-5-ureidovalerylamino]benzoyl]-3-azetidinecarboxylic acid | |
| 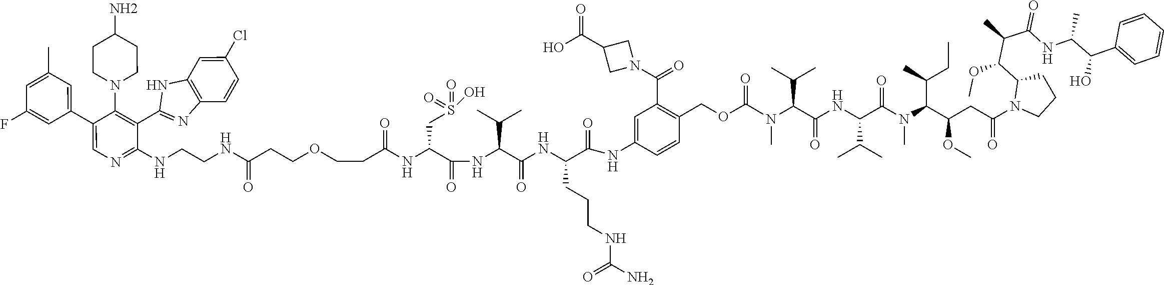 [SMILES: CC[C@H](C)[C@@H]([C@@H](CC(=O)N1CCC[C@H]1[C@H](OC)[C@@H](C)C(=O)N[C@H](C)[C@@H](O)c1ccccc1)OC)N(C)C(=O)[C@@H](NC(=O)[C@H](C(C)C)N(C)C(=O)OCc1ccc(NC(=O)[C@H](CCCNC(N)=O)NC(=O)[C@@H](NC(=O)[C@@H](CS(=O)(=O)O)NC(=O)CCOCCC(=O)NCCNc2ncc(-c3cc(C)cc(F)c3)c(N3CCC(N)CC3)c2-c2nc3ccc(Cl)cc3[nH]2)C(C)C)cc1C(=O)N1CC(C(=O)O)C1)C(C)C] | |
| (Compound 77) | |
| 1-[2-({[(S)-1-[N-(S)-1-{[(1S,2R)-4-[(S)-2-{(1R,2R)-2-[N-(1R,2S)-2-hydroxy-1-methyl-2-phenylethylcarbamoyl]-1-methoxypropyl}-1- | 1934.9 |
| pyrrolidinyl]-1-[(S)-1-methylpropyl]-2-methoxy-4-oxobutyl]-N-methylcarbamoyl}-2-methylpropylcarbamoyl]-2- | |
| methylpropyl](methyl)(aminocarbonyloxy)}methyl)-5-[(S)-2-[(S)-2-[(S)-2-[3-(2-{N-2-[4-(4-amino-1-piperidyl)-3-(6-chloro-1H-1,3- | |
| benzimidazol-2-yl)-5-(3-fluoro-5-tolyl)-2-pyridylamino]ethylcarbamoyl}ethoxy)propionylamino]-3-sulfopropionylamino]-3- | |
| methylbutyrylamino]propionylamino]benzoyl]-3-azetidinecarboxylic acid | |
| 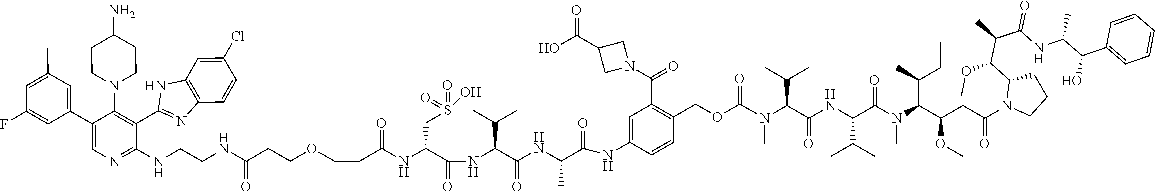 [SMILES: CC[C@H](C)[C@@H]([C@@H](CC(=O)N1CCC[C@H]1[C@H](OC)[C@@H](C)C(=O)N[C@H](C)[C@@H](O)c1ccccc1)OC)N(C)C(=O)[C@@H](NC(=O)[C@H](C(C)C)N(C)C(=O)OCc1ccc(NC(=O)[C@H](C)NC(=O)[C@@H](NC(=O)[C@@H](CS(=O)(=O)O)NC(=O)CCOCCC(=O)NCCNc2ncc(-c3cc(C)cc(F)c3)c(N3CCC(N)CC3)c2-c2nc3ccc(Cl)cc3[nH]2)C(C)C)cc1C(=O)N1CC(C(=O)O)C1)C(C)C] | |
| (Compound 78) | |
| (S)-4-[N-(S)-1-[N-(S)-1-[N-3-(N-2-aminoethylcarbamoyl)-4-({[(S)-1-[N-(S)-1-{[(1S,2R)-4-[(S)-2-{(1R,2R)-2-[N-(1R,2S)-2-hydroxy- | 1972.0 |
| 1-methyl-2-phenylethylcarbamoyl]-1-methoxypropyl}-1-pyrrolidinyl]-1-[(S)-1-methylpropyl]-2-methoxy-4-oxobutyl]-N- | |
| methylcarbamoyl}-2-methylpropylcarbamoyl]-2-methylpropyl](methyl)(aminocarbonyloxy)}methyl)phenylcarbamoyl]-4- | |
| ureidobutylcarbamoyl]-2-methylpropylcarbamoyl]-4-[3-(2-{N-2-[4-(4-amino-1-piperidyl)-3-(5-fluoro-7-methoxy-1H-1,3- | |
| benzimidazol-2-yl)-5-(3-fluoro-5-tolyl)-2-pyridylamino]ethylcarbamoyl}ethoxy)propionylamino]butyric acid | |
| 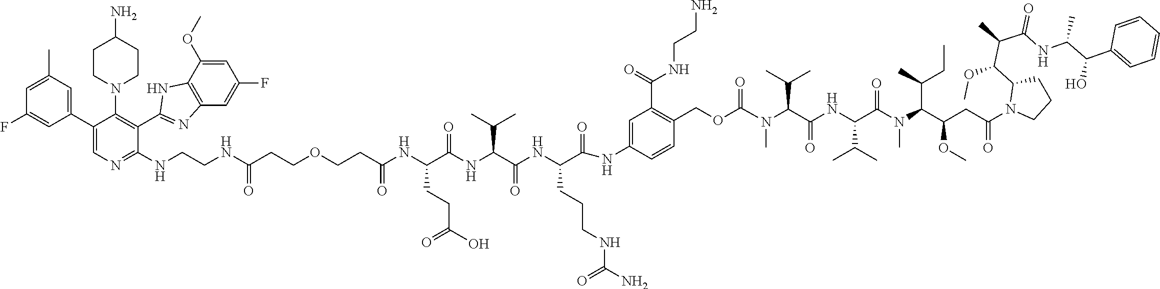 [SMILES: CC[C@H](C)[C@@H]([C@@H](CC(=O)N1CCC[C@H]1[C@H](OC)[C@@H](C)C(=O)N[C@H](C)[C@@H](O)c1ccccc1)OC)N(C)C(=O)[C@@H](NC(=O)[C@H](C(C)C)N(C)C(=O)OCc1ccc(NC(=O)[C@H](CCCNC(N)=O)NC(=O)[C@@H](NC(=O)[C@H](CCC(=O)O)NC(=O)CCOCCC(=O)NCCNc2ncc(-c3cc(C)cc(F)c3)c(N3CCC(N)CC3)c2-c2nc3cc(F)cc(OC)c3[nH]2)C(C)C)cc1C(=O)NCCN)C(C)C] | |
| (Compound 79) | |
| (S)-4-[N-(S)-1-[N-(S)-1-[N-3-(N-2-aminoethylcarbamoyl)-4-({[(S)-1-[N-(S)-1-{[(1S,2R)-4-[(S)-2-{(1R,2R)-2-[N-(1R,2S)-2-hydroxy- | 2000.0 |
| 1-methyl-2-phenylethylcarbamoyl]-1-methoxypropyl}-1-pyrrolidinyl]-1-[(S)-1-methylpropyl]-2-methoxy-4-oxobutyl]-N- | |
| methylcarbamoyl}-2-methylpropylcarbamoyl]-2-methylpropyl](methyl)(aminocarbonyloxy)}methyl)phenylcarbamoyl]-4- | |
| ureidobutylcarbamoyl]-2-methylpropylcarbamoyl]-4-{3-[2-(N-2-{4-[(4aR,8aR)-octahydro-1H-4-oxa-1,6-diazanaphth-6-yl]-3-(6- | |
| chloro-1H-1,3-benzimidazol-2-yl)-5-(3-fluoro-5-tolyl)-2-pyridylamino}ethylcarbamoyl)ethoxy]propionylamino}butyric acid | |
| 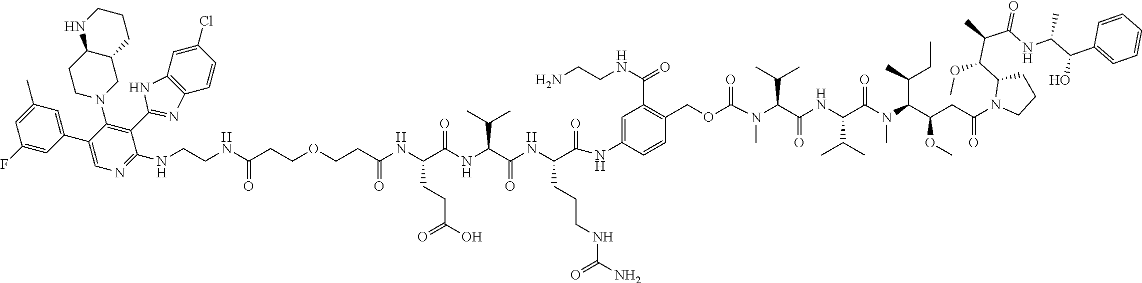 [SMILES: CC[C@H](C)[C@@H]([C@@H](CC(=O)N1CCC[C@H]1[C@H](OC)[C@@H](C)C(=O)N[C@H](C)[C@@H](O)c1ccccc1)OC)N(C)C(=O)[C@@H](NC(=O)[C@H](C(C)C)N(C)C(=O)OCc1ccc(NC(=O)[C@H](CCCNC(N)=O)NC(=O)[C@@H](NC(=O)[C@H](CCC(=O)O)NC(=O)CCOCCC(=O)NCCNc2ncc(-c3cc(C)cc(F)c3)c(N3CC[C@H]4NCCC[C@@H]4C3)c2-c2nc3ccc(Cl)cc3[nH]2)C(C)C)cc1C(=O)NCCN)C(C)C] | |
| (Compound 80) | |
| 1-(S)-1-[N-(S)-1-{[(1S,2R)-4-[(S)-2-{(1R,2R)-2-[N-(1R,2S)-2-hydroxy-1-methyl-2-phenylethylcarbamoyl]-1-methoxypropyl}-1- | 1926.0 |
| pyrrolidinyl]-1-[(S)-1-methylpropyl]-2-methoxy-4-oxobutyl]-N-methylcarbamoyl}-2-methylpropylcarbamoyl]-2-methylpropyl 1- | |
| methyl [2-(N-2-aminoethylcarbamoyl)-4-[(S)-2-[(S)-2-{1-[N-2-(2-{N-2-[4-(4-amino-1-piperidyl)-3-(6-chloro-1H-1,3-benzimidazol-2- | |
| yl)-5-(3-fluoro-5-tolyl)-2-pyridylamino]ethylcarbamoyl}ethoxy)ethylcarbamoyl]cyclovalerylamino}-3-methylbutyrylamino]-5- | |
| ureidovalerylamino]phenyl]methanecarbamate | |
| 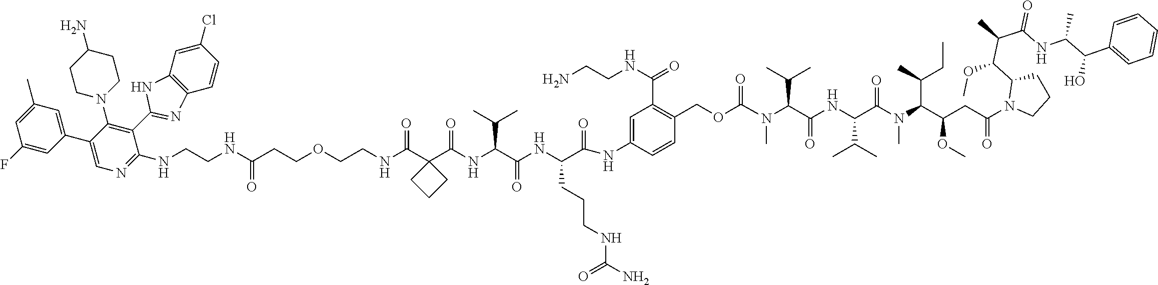 [SMILES: CC[C@H](C)[C@@H]([C@@H](CC(=O)N1CCC[C@H]1[C@H](OC)[C@@H](C)C(=O)N[C@H](C)[C@@H](O)c1ccccc1)OC)N(C)C(=O)[C@@H](NC(=O)[C@H](C(C)C)N(C)C(=O)OCc1ccc(NC(=O)[C@H](CCCNC(N)=O)NC(=O)[C@@H](NC(=O)C2(C(=O)NCCOCCC(=O)NCCNc3ncc(-c4cc(C)cc(F)c4)c(N4CCC(N)CC4)c3-c3nc4ccc(Cl)cc4[nH]3)CCC2)C(C)C)cc1C(=O)NCCN)C(C)C] | |
| (Compound 81) | |
| 1-(S)-1-[N-(S)-1-{[(1S,2R)-4-[(S)-2-{(1R,2R)-2-[N-(1R,2S)-2-hydroxy-1-methyl-2-phenylethylcarbamoyl]-1-methoxypropyl}-1- | 1840.0 |
| pyrrolidinyl]-1-[(S)-1-methylpropyl]-2-methoxy-4-oxobutyl]-N-methylcarbamoyl}-2-methylpropylcarbamoyl]-2-methylpropyl 1- | |
| methyl [2-(N-2-aminoethylcarbamoyl)-4-[(S)-2-[(S)-2-{1-[N-2-(2-{N-2-[4-(4-amino-1-piperidyl)-3-(6-chloro-1H-1,3-benzimidazol-2- | |
| yl)-5-(3-fluoro-5-tolyl)-2-pyridylamino]ethylcarbamoyl}ethoxy)ethylcarbamoyl]cyclovalerylamino}-3- | |
| methylbutyrylamino]propionylamino]phenyl]methanecarbamate | |
| 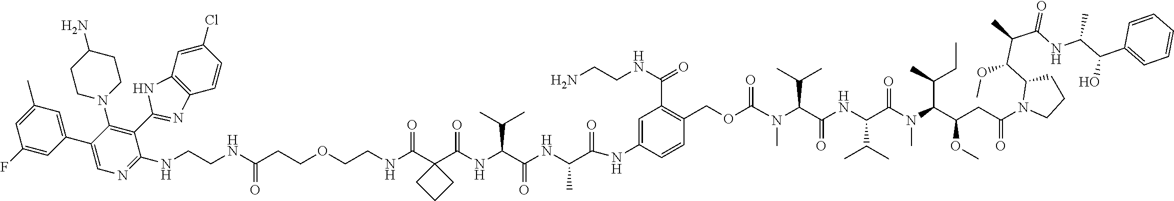 [SMILES: CC[C@H](C)[C@@H]([C@@H](CC(=O)N1CCC[C@H]1[C@H](OC)[C@@H](C)C(=O)N[C@H](C)[C@@H](O)c1ccccc1)OC)N(C)C(=O)[C@@H](NC(=O)[C@H](C(C)C)N(C)C(=O)OCc1ccc(NC(=O)[C@H](C)NC(=O)[C@@H](NC(=O)C2(C(=O)NCCOCCC(=O)NCCNc3ncc(-c4cc(C)cc(F)c4)c(N4CCC(N)CC4)c3-c3nc4ccc(Cl)cc4[nH]3)CCC2)C(C)C)cc1C(=O)NCCN)C(C)C] | |
| (Compound 82) | |
| 11-{N-2-[2-({methyl[(S)-2-methyl-1-[N-(S)-1-{[(1S,2R)-2-methoxy-4-[(S)-2-[(1R,2R)-1-methoxy-2-[N-(1R,2S)-2-hydroxy-1-methyl- | 2805.1 |
| 2-phenylethylcarbamoyl]propyl]-1-pyrrolidinyl]-1-[(S)-1-methylpropyl]-4-oxobutyl]-N-methylcarbamoyl}-2- | |
| methylpropylcarbamoyl]propyl](aminocarbonyloxy)}methyl)-5-[(S)-2-[(S)-3-methyl-2-{3-[2-(2-{N-2-[4-(4-amino-1-piperidyl)-3-(6- | |
| chloro-1H-1,3-benzimidazol-2-yl)-5-(3-fluoro-5-tolyl)-2- | |
| pyridylamino]ethylcarbamoyl}ethoxy)ethoxy]propionylamino}butyrylamino]-5- | |
| ureidovalerylamino]benzylamino]ethylcarbamoyl}undecanoic acid | |
| 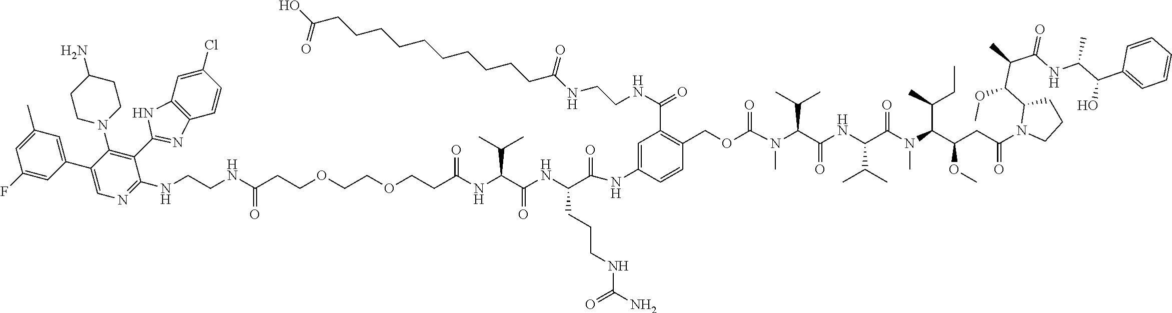 [SMILES: CC[C@H](C)[C@@H]([C@@H](CC(=O)N1CCC[C@H]1[C@H](OC)[C@@H](C)C(=O)N[C@H](C)[C@@H](O)c1ccccc1)OC)N(C)C(=O)[C@@H](NC(=O)[C@H](C(C)C)N(C)C(=O)OCc1ccc(NC(=O)[C@H](CCCNC(N)=O)NC(=O)[C@@H](NC(=O)CCOCCOCCC(=O)NCCNc2ncc(-c3cc(C)cc(F)c3)c(N3CCC(N)CC3)c2-c2nc3ccc(Cl)cc3[nH]2)C(C)C)cc1C(=O)NCCNC(=O)CCCCCCCCCCC(=O)O)C(C)C] | |
| (Compound 83) | |
| 1-(S)-1-[N-(S)-1-{[(1S,2R)-4-[(S)-2-{(1R,2R)-2-[N-(1R,2S)-2-hydroxy-1-methyl-2-phenylethylcarbamoyl]-1-methoxypropyl}-1- | 1982.0 |
| pyrrolidinyl]-1-[(S)-1-methylpropyl]-2-methoxy-4-oxobutyl]-N-methylcarbamoyl}-2-methylpropylcarbamoyl]-2-methylpropyl 1- | |
| methyl [2-(N-2-aminoethylcarbamoyl)-4-[(S)-2-[(S)-2-[(S)-2-[3-(2-{N-2-[4-(4-amino-1-piperidyl)-3-(6-chloro-1H-1,3-benzimidazol-2- | |
| yl)-5-(3-fluoro-5-tolyl)-2-pyridylamino]ethylcarbamoyl}ethoxy)propionylamino]-4-(1H-tetraazol-5-yl)butyrylamino]-3- | |
| methylbutyrylamino]-5-ureidovalerylamino]phenyl]methanecarbamate | |
| 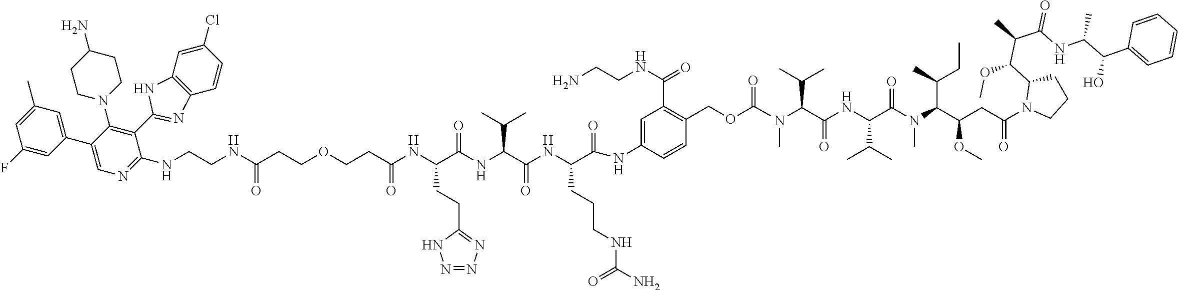 [SMILES: CC[C@H](C)[C@@H]([C@@H](CC(=O)N1CCC[C@H]1[C@H](OC)[C@@H](C)C(=O)N[C@H](C)[C@@H](O)c1ccccc1)OC)N(C)C(=O)[C@@H](NC(=O)[C@H](C(C)C)N(C)C(=O)OCc1ccc(NC(=O)[C@H](CCCNC(N)=O)NC(=O)[C@@H](NC(=O)[C@H](CCc2nnn[nH]2)NC(=O)CCOCCC(=O)NCCNc2ncc(-c3cc(C)cc(F)c3)c(N3CCC(N)CC3)c2-c2nc3ccc(Cl)cc3[nH]2)C(C)C)cc1C(=O)NCCN)C(C)C] | |
| (Compound 84) | |
| 1-(S)-1-[N-(S)-1-{[(1S,2R)-4-[(S)-2-{(1R,2R)-2-[N-(1R,2S)-2-hydroxy-1-methyl-2-phenylethylcarbamoyl]-1-methoxypropyl}-1- | 2015.0 |
| pyrrolidinyl]-1-[(S)-1-methylpropyl]-2-methoxy-4-oxobutyl]-N-methylcarbamoyl}-2-methylpropylcarbamoyl]-2-methylpropyl 1- | |
| methyl [2-(N-2-aminoethylcarbamoy])-4-[(S)-2-[(S)-2-[(S)-2-[3-(2-{N-2-[4-(4-amino-1-piperidyl)-3-(6-chloro-1H-1,3-benzimidazol-2- | |
| yl)-5-(3-fluoro-5-tolyl)-2-pyridylamino]ethylcarbamoyl}ethoxy)propionylamino]-3-(3-indolyl)propionylamino]-3- | |
| methylbutyrylamino]-5-ureidovalerylamino]phenyl\|methanecarbamate | |
| 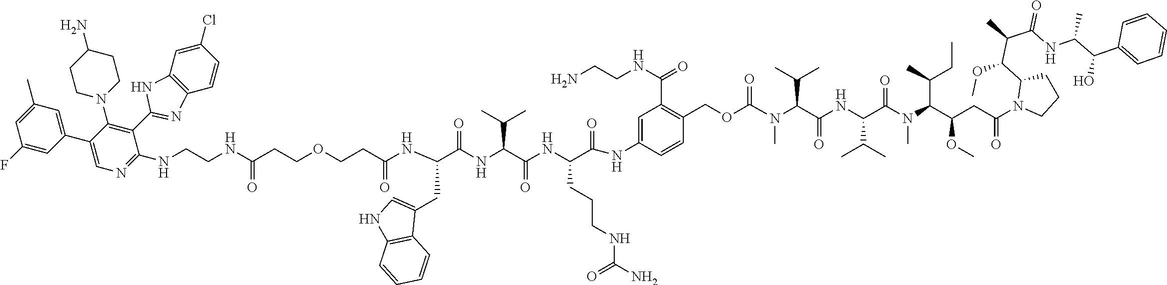 [SMILES: CC[C@H](C)[C@@H]([C@@H](CC(=O)N1CCC[C@H]1[C@H](OC)[C@@H](C)C(=O)N[C@H](C)[C@@H](O)c1ccccc1)OC)N(C)C(=O)[C@@H](NC(=O)[C@H](C(C)C)N(C)C(=O)OCc1ccc(NC(=O)[C@H](CCCNC(N)=O)NC(=O)[C@@H](NC(=O)[C@H](Cc2c[nH]c3ccccc23)NC(=O)CCOCCC(=O)NCCNc2ncc(-c3cc(C)cc(F)c3)c(N3CCC(N)CC3)c2-c2nc3ccc(Cl)cc3[nH]2)C(C)C)cc1C(=O)NCCN)C(C)C] | |
| (Compound 85) | |
| [2-({[(S)-1-[N-(S)-1-{[(1S,2R)-4-[(S)-2-{(1R,2R)-2-[N-(1R,2S)-2-hydroxy-1-methyl-2-phenylethylcarbamoyl]-1-methoxypropyl}-1- | 1975.9 |
| pyrrolidinyl]-1-[(S)-1-methylpropyl]-2-methoxy-4-oxobutyl]-N-methylcarbamoyl}-2-methylpropylcarbamoyl]-2- | |
| methylpropyl](methyl)(aminocarbonyloxy)}methyl)-5-[(S)-2-[(S)-2-(14-{N-2-[4-(4-amino-1-piperidyl)-3-(6-chloro-1H-1,3- | |
| benzimidazol-2-yl)-5-(3-fluoro-5-tolyl)-2-pyridylamino]ethylcarbamoyl}-3,6,9,12-tetraoxatetradecylcarbonylamino)-3- | |
| methylbutyrylamino]-5-ureidovalerylamino]benzylamino]acetic acid | |
| 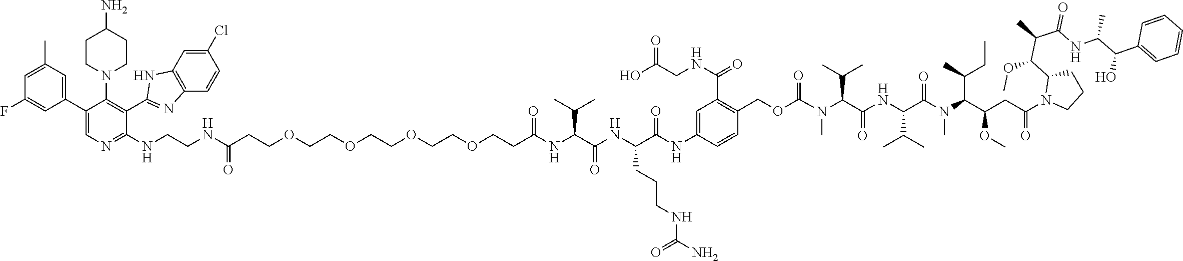 [SMILES: CC[C@H](C)[C@@H]([C@@H](CC(=O)N1CCC[C@H]1[C@H](OC)[C@@H](C)C(=O)N[C@H](C)[C@@H](O)c1ccccc1)OC)N(C)C(=O)[C@@H](NC(=O)[C@H](C(C)C)N(C)C(=O)OCc1ccc(NC(=O)[C@H](CCCNC(N)=O)NC(=O)[C@@H](NC(=O)CCOCCOCCOCCOCCC(=O)NCCNc2ncc(-c3cc(C)cc(F)c3)c(N3CCC(N)CC3)c2-c2nc3ccc(Cl)cc3[nH]2)C(C)C)cc1C(=O)NCC(=O)O)C(C)C] | |
| (Compound 86) | |
| 1-(S)-1-[N-(S)-1-{[(1S,2R)-4-[(S)-2-{(1R,2R)-2-[N-(1R,2S)-2-hydroxy-1-methyl-2-phenylethylcarbamoyl]-1-methoxypropyl}-1- | 1816.0 |
| pyrrolidinyl]-1-[(S)-1-methylpropyl]-2-methoxy-4-oxobutyl]-N-methylcarbamoyl}-2-methylpropylcarbamoyl]-2-methylpropyl 1- | |
| methyl [2-(N-methoxycarbamoyl)-4-[(S)-2-[(S)-2-[3-(2-{N-2-[4-(4-amino-1-piperidyl)-3-(6-chloro-1H-1,3-benzimidazol-2-yl)-5-(3- | |
| fluoro-5-tolyl)-2-pyridylamino]ethylcarbamoyl}ethoxy)propionylamino]-3-methylbutyrylamino]-5- | |
| ureidovalerylamino]phenyl]methanecarbamate | |
| 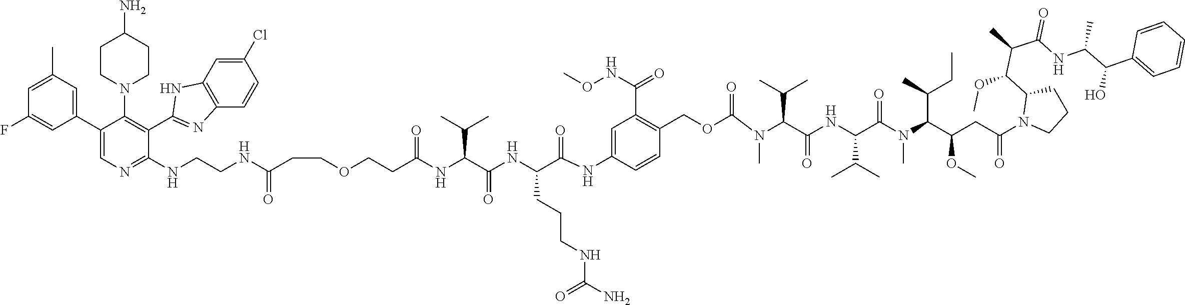 [SMILES: CC[C@H](C)[C@@H]([C@@H](CC(=O)N1CCC[C@H]1[C@H](OC)[C@@H](C)C(=O)N[C@H](C)[C@@H](O)c1ccccc1)OC)N(C)C(=O)[C@@H](NC(=O)[C@H](C(C)C)N(C)C(=O)OCc1ccc(NC(=O)[C@H](CCCNC(N)=O)NC(=O)[C@@H](NC(=O)CCOCCC(=O)NCCNc2ncc(-c3cc(C)cc(F)c3)c(N3CCC(N)CC3)c2-c2nc3ccc(Cl)cc3[nH]2)C(C)C)cc1C(=O)NOC)C(C)C] | |
| (Compound 87) | |
| 1-(S)-1-[N-(S)-1-{[(1S,2R)-4-[(S)-2-{(1R,2R)-2-[N-(1R,2S)-2-hydroxy-1-methyl-2-phenylethylcarbamoyl]-1-methoxypropyl}-1- | 1845.0 |
| pyrrolidinyl]-1-[(S)-1-methylpropyl]-2-methoxy-4-oxobutyl]-N-methylcarbamoyl}-2-methylpropylcarbamoyl]-2-methylpropyl 1- | |
| methyl [2-(N-2-aminoethoxycarbamoyl)-4-[(S)-2-[(S)-2-[3-(2-{N-2-[4-(4-amino-1-piperidyl)-3-(6-chloro-1H-1,3-benzimidazol-2-yl)- | |
| 5-(3-fluoro-5-tolyl)-2-pyridylamino]ethylcarbamoyl}ethoxy)propionylamino]-3-methylbutyrylamino]-5- | |
| ureidovalerylamino]phenyl]methanecarbamate | |
| 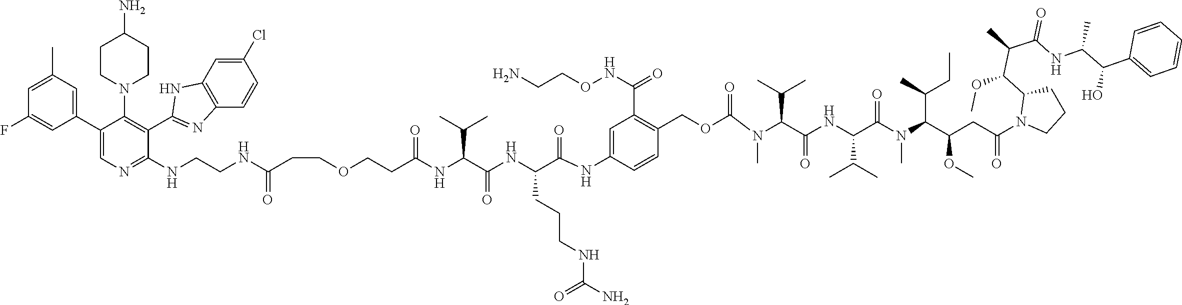 [SMILES: CC[C@H](C)[C@@H]([C@@H](CC(=O)N1CCC[C@H]1[C@H](OC)[C@@H](C)C(=O)N[C@H](C)[C@@H](O)c1ccccc1)OC)N(C)C(=O)[C@@H](NC(=O)[C@H](C(C)C)N(C)C(=O)OCc1ccc(NC(=O)[C@H](CCCNC(N)=O)NC(=O)[C@@H](NC(=O)CCOCCC(=O)NCCNc2ncc(-c3cc(C)cc(F)c3)c(N3CCC(N)CC3)c2-c2nc3ccc(Cl)cc3[nH]2)C(C)C)cc1C(=O)NOCCN)C(C)C] | |
| (Compound 88) | |
| (S)-4-[N-(S)-1-[N-(S)-1-[N-3-(N-methoxycarbamoyl)-4-({[(S)-1-[N-(S)-1-{[(1S,2R)-4-[(S)-2-{(1R,2R)-2-[N-(1R,2S)-2-hydroxy-1- | 1945.0 |
| methyl-2-phenylethylcarbamoyl]-1-methoxypropyl}-1-pyrrolidinyl]-1-[(S)-1-methylpropyl]-2-methoxy-4-oxobutyl]-N- | |
| methylcarbamoyl}-2-methylpropylcarbamoyl]-2-methylpropyl](methyl)(aminocarbonyloxy)}methyl)phenylcarbamoyl]-4- | |
| ureidobutylcarbamoyl]-2-methylpropylcarbamoyl]-4-[3-(2-{N-2-[4-(4-amino-1-piperidyl)-3-(6-chloro-1H-1,3-benzimidazol-2-yl)-5- | |
| (3-fluoro-5-tolyl)-2-pyridylamino]ethylcarbamoyl}ethoxy)propionylamino]butyric acid | |
| 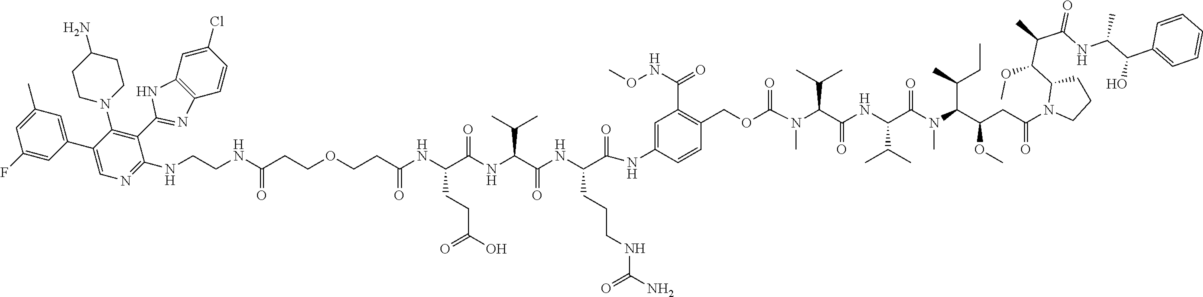 [SMILES: CC[C@H](C)[C@@H]([C@@H](CC(=O)N1CCC[C@H]1[C@H](OC)[C@@H](C)C(=O)N[C@H](C)[C@@H](O)c1ccccc1)OC)N(C)C(=O)[C@@H](NC(=O)[C@H](C(C)C)N(C)C(=O)OCc1ccc(NC(=O)[C@H](CCCNC(N)=O)NC(=O)[C@@H](NC(=O)[C@H](CCC(=O)O)NC(=O)CCOCCC(=O)NCCNc2ncc(-c3cc(C)cc(F)c3)c(N3CCC(N)CC3)c2-c2nc3ccc(Cl)cc3[nH]2)C(C)C)cc1C(=O)NOC)C(C)C] | |
| (Compound 89) | |
| (S)-4-[N-(S)-1-[N-(S)-1-[N-3-(N-2-aminoethoxycarbamoyl)-4-({[(S)-1-[N-(S)-1-{[(1S,2R)-4-[(S)-2-{(1R,2R)-2-[N-(1R,2S)-2- | 1974.0 |
| hydroxy-1-methyl-2-phenylethylcarbamoyl]-1-methoxypropyl}-1-pyrrolidinyl)-1-[(S)-1-methylpropyl]-2-methoxy-4-oxobutyl]-N- | |
| methylcarbamoyl}-2-methylpropylcarbamoyl]-2-methylpropyl](methyl)(aminocarbonyloxy)}methyl)phenylcarbamoyl]-4- | |
| ureidobutylcarbamoyl]-2-methylpropylcarbamoyl]-4-[3-(2-{N-2-[4-(4-amino-1-piperidyl)-3-(6-chloro-1H-1,3-benzimidazol-2-yl)-5- | |
| (3-fluoro-5-tolyl)-2-pyridylamino]ethylcarbamoyl}ethoxy)propionylamino]butyric acid | |
| 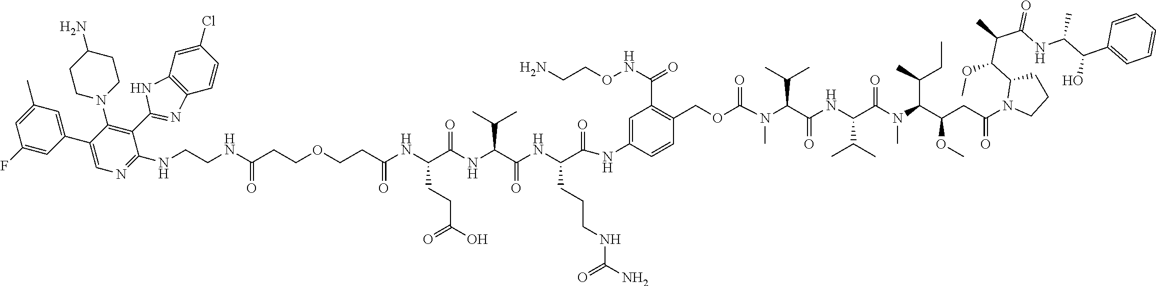 [SMILES: CC[C@H](C)[C@@H]([C@@H](CC(=O)N1CCC[C@H]1[C@H](OC)[C@@H](C)C(=O)N[C@H](C)[C@@H](O)c1ccccc1)OC)N(C)C(=O)[C@@H](NC(=O)[C@H](C(C)C)N(C)C(=O)OCc1ccc(NC(=O)[C@H](CCCNC(N)=O)NC(=O)[C@@H](NC(=O)[C@H](CCC(=O)O)NC(=O)CCOCCC(=O)NCCNc2ncc(-c3cc(C)cc(F)c3)c(N3CCC(N)CC3)c2-c2nc3ccc(Cl)cc3[nH]2)C(C)C)cc1C(=O)NOCCN)C(C)C] | |
| (Compound 90) | |
| (S)-4-[N-(S)-1-[N-(S)-1-[N-4-({[(S)-1-[N-(S)-1-{[(1S,2R)-4-[(S)-2-{(1R,2R)-2-[N-(1R,2S)-2-hydroxy-1-methyl-2- | 1970.0 |
| phenylethylcarbamoyl]-1-methoxypropyl}-1-pyrrolidinyl]-1-[(S)-1-methylpropyl]-2-methoxy-4-oxobutyl]-N-methylcarbamoyl}-2- | |
| methylpropylcarbamoyl]-2-methylpropyl](methyl)(aminocarbonyloxy)} methyl)-3-[(3-amino-1-azetidinyl)carbonyl]phenylcarbamoyl]- | |
| 4-ureidobutylcarbamoyl]-2-methylpropylcarbamoyl]-4-[3-(2-{N-2-[4-(4-amino-1-piperidyl)-3-(6-chloro-1H-1,3-benzimidazol-2-y])-5- | |
| (3-fluoro-5-tolyl)-2-pyridylamino]ethylcarbamoyl}ethoxy)propionylamino]butyric acid | |
| 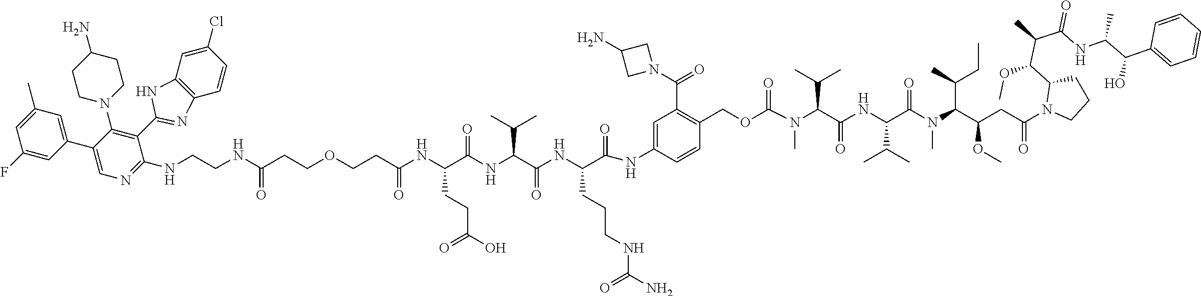 [SMILES: CC[C@H](C)[C@@H]([C@@H](CC(=O)N1CCC[C@H]1[C@H](OC)[C@@H](C)C(=O)N[C@H](C)[C@@H](O)c1ccccc1)OC)N(C)C(=O)[C@@H](NC(=O)[C@H](C(C)C)N(C)C(=O)OCc1ccc(NC(=O)[C@H](CCCNC(N)=O)NC(=O)[C@@H](NC(=O)[C@H](CCC(=O)O)NC(=O)CCOCCC(=O)NCCNc2ncc(-c3cc(C)cc(F)c3)c(N3CCC(N)CC3)c2-c2nc3ccc(Cl)cc3[nH]2)C(C)C)cc1C(=O)N1CC(N)C1)C(C)C] | |
| (Compound 91) | |

### Example D. (2S,3S,4S,5R,6S)-6-[5-({[(S)-1-[N—(S)-1-{[(1S,2R)-4-[(S)-2-{1R,2R)-2-[N-(1R,2S)-2-hydroxy-1-methyl-2-phenylethylcarbamoyl]-1-methoxypropyl}-1-pyrrolidinyl]-1-[(S)-1-methylpropyl]-2-methoxy-4-oxobutyl]-N-methylcarbamoyl}-2-methylpropylcarbamoyl]-2-methylpropyl](methyl)aminocarbonyloxy)}methyl)-2-[(S)-2-[(S)-2-[(R)-3-[N-2-(2-{N-2-[4-(4-amino-1-piperidyl)-3-(6-chloro-1H-1,3-benzimidazol-2-yl)-5-(3-fluoro-5-tolyl)-2-pyridylamino]ethylcarbamoyl}ethoxy)ethylcarbamoyl]-3-aminopropionylamino]-3-methylbutyrylamino]-5-ureidovalerylamino]phenoxy]-3,4,5-trihydroxytetrahydro-2H-pyran-2-carboxylic acid (Compound 92)

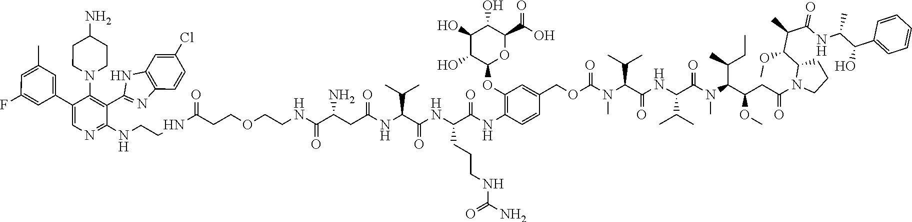 [SMILES: CC[C@H](C)[C@@H]([C@@H](CC(=O)N1CCC[C@H]1[C@H](OC)[C@@H](C)C(=O)N[C@H](C)[C@@H](O)c1ccccc1)OC)N(C)C(=O)[C@@H](NC(=O)[C@H](C(C)C)N(C)C(=O)OCc1ccc(NC(=O)[C@H](CCCNC(N)=O)NC(=O)[C@@H](NC(=O)C[C@@H](N)C(=O)NCCOCCC(=O)NCCNc2ncc(-c3cc(C)cc(F)c3)c(N3CCC(N)CC3)c2-c2nc3ccc(Cl)cc3[nH]2)C(C)C)c(O[C@@H]2O[C@H](C(=O)O)[C@@H](O)[C@H](O)[C@H]2O)c1)C(C)C]

Step D-1, preparation of tert-butyl (1-(2-((1-(9H-fluoren-9-yl)-3,10-dioxo-2,7-dioxa-4,11-diazatridecan-13-yl)amino)-3-(6-chloro-1H-benzo[d]imidazol-2-yl)-5-(3-fluoro-5-methylphenyl)pyridin-4-yl)piperidin-4-yl)carbamate: Into a 40-mL vial was placed 3-(2-((((9H-fluoren-9-yl)methoxy)carbonyl)amino)ethoxy)propanoic acid (450 mg, 1.50 Eq, 1.27 mmol), 2-(2,5-dioxopyrrolidin-1-yl)-1,1,3,3-tetramethyluronium hexafluorophosphate(V) (453 mg, 1.50 Eq, 1.26 mmol), Diisopropylethylamine (330 mg, 0.44 mL, 3.03 Eq, 2.55 mmol) and DMF (5 mL). The reaction mixture was stirred at 25° C. for 10 minutes, then tert-butyl (1-(2-((2-aminoethyl)amino)-3-(6-chloro-1H-benzo[d]imidazol-2-yl)-5-(3-fluoro-5-methylphenyl)pyridin-4-yl)piperidin-4-yl)carbamate (500 mg, 1 Eq, 842 μmol) was added and the reaction mixture was stirred at 25° C. for an additional hour to provide tert-butyl (1-(2-((1-(9H-fluoren-9-yl)-3,10-dioxo-2,7-dioxa-4,11-diazatridecan-13-yl)amino)-3-(6-chloro-1H-benzo[d]imidazol-2-yl)-5-(3-fluoro-5-methylphenyl)pyridin-4-yl)piperidin-4-yl)carbamate (520 mg, 558 μmol, 66.3%); (M+H)⁺=931.4. The reaction system was not purified and directly put into the next step.

Step D-2, preparation of tert-butyl (1-(2-((2-(3-(2-aminoethoxy)propanamido)ethyl)amino)-3-(6-chloro-1H-benzo[d]imidazol-2-yl)-5-(3-fluoro-5-methylphenyl)pyridin-4-yl)piperidin-4-yl)carbamate: Into a 40-mL vial was placed tert-butyl (1-(2-((1-(9H-fluoren-9-yl)-3,10-dioxo-2,7-dioxa-4,11-diazatridecan-13-yl)amino)-3-(6-chloro-1H-benzo[d]imidazol-2-yl)-5-(3-fluoro-5-methylphenyl)pyridin-4-yl)piperidin-4-yl)carbamate (520 mg, 1 Eq. 558 μmol) in DMF (5 mL), and 1,8-Diazabicydo[5.4.0]undec-7-ene (255 mg, 250 μL, 3.00 Eq, 1.67 mmol). The reaction mixture was stirred at 25° C. for 30 minutes. The mixture was directly purified by MPLC with the following conditions: Column, WelFlash™, C18 120 g, Spherical 20-40 μm; Mobile phase, Water (0.1% TFA) and ACN (5% ACN to 5% ACN in 1 min, 30% ACN up to 80% in 8 min, 95% ACN to 95% in 3 min); Total flow rate, 70 mL/min; Detector, UV 220 nm. This provided tert-butyl (1-(2-((2-(3-(2-aminoethoxy)propanamido)ethyl)amino)-3-(6-chloro-1H-benzo[d]imidazol-2-yl)-5-(3-fluoro-5-methylphenyl)pyridin-4-yl)piperidin-4-yl)carbamate (350 mg, 493 μmol, 88.4%); (M+H)⁺=709.3.

Step D-3, preparation of tert-butyl (R)-3-((tert-butoxycarbonyl)amino)-4-((2-(3-((2-((4-(4-((tert-butoxycarbonyl)amino)piperidin-1-yl)-3-(6-chloro-1H-benzo[d]imidazol-2-yl)-5-(3-fluoro-5-methylphenyl)pyridin-2-yl)amino)ethyl)amino)-3-oxopropoxy)ethyl)amino)-4-oxobutanoate: Into a 8-mL vial was placed (R)-4-(tert-butoxy)-2-((tert-butoxycarbonyl)amino)-4-oxobutanoic acid (306 mg, 1.50 Eq, 1.06 mmol), N,N,n,n-tetramethyl-o-(n-succinimidyl)uronium hexafluorophosphate (380 mg, 1.50 Eq, 1.06 mmol), Diisopropylethylamine (275 mg, 366 μL, 3.02 Eq, 2.13 mmol), and DMF (5 mL). The resulting reaction mixture was stirred for 10 minutes at 25° C. Then to this was added tert-butyl (1-(2-((2-(3-(2-aminoethoxy)propanamido)ethyl)amino)-3-(6-chloro-1H-benzo[d]imidazol-2-yl)-5-(3-fluoro-5-methylphenyl)pyridin-4-yl)piperidin-4-yl)carbamate (500 mg, 1 Eq, 705 μmol). The resulting reaction mixture was stirred for 1 hour at 25° C. The crude product was purified by Prep-HPLC with the following conditions: Column: SunFire prep OBD 19*150 mm 5 um; Mobile Phase A: Water (0.05% TFA); Mobile Phase B: ACN; Gradient: 25% B to 65% B in 8 min; Flow rate:50 mL/min; Wave Length: 220 nm. The collected fractions were dried by lyophilization to provide tert-butyl (R)-3-((tert-butoxycarbonyl)amino)-4-((2-(3-((2-((4-(4-((tert-butoxycarbonyl)amino)piperidin-1-yl)-3-(6-chloro-1H-benzo[d]imidazol-2-yl)-5-(3-fluoro-5-methylphenyl)pyridin-2-yl)amino)ethyl)amino)-3-oxopropoxy)ethyl)amino)-4-oxobutanoate (500 mg, 510 μmol, 72.3%); (M+H)⁺=980.5.

Step D-4, preparation of (R)-3-((tert-butoxycarbonyl)amino)-4-((2-(3-((2-((4-(4-((tert-butoxy carbonyl)amino)piperidin-1-yl)-3-(6-chloro-1H-benzo[d]imidazol-2-yl)-5-(3-fluoro-5-methylphenyl)pyridin-2-yl)amino)ethyl)amino)-3-oxopropoxy)ethyl)amino)-4-oxobutanoic acid: Into a 40-mL vial was placed a mixture of tert-butyl (R)-3-((tert-butoxycarbonyl)amino)-4-((2-(3-((2-((4-(4-((tert-butoxycarbonyl)amino)piperidin-1-yl)-3-(6-chloro-1H-benzo[d]imidazol-2-yl)-5-(3-fluoro-5-methylphenyl)pyridin-2-yl)amino)ethyl)amino)-3-oxopropoxy)ethyl)amino)-4-oxobutanoate (500 mg, 1 Eq, 510 μmol), lithium hydroxide (61 mg, 5.0 Eq. 2.5 mmol), MeOH (5 mL) and H₂O (5 mL). The reaction mixture was stirred at 40° C. for 1 hour. The mixture was directly purified by MPLC with the following conditions: Column, WelFlash™, C18 120 g, Spherical 20-40 μm; Mobile phase, Water (0.1% TFA) and ACN (5% ACN to 5% ACN in 1 min, 30% ACN up to 80% in 8 min, 95% ACN to 95% in 3 min); Total flow rate, 70 mL/min; Detector, UV 220 nm. This provided (R)-3-((tert-butoxycarbonyl)amino)-4-((2-(3-((2-((4-(4-((tert-butoxycarbonyl)amino)piperidin-1-yl)-3-(6-chloro-1H-benzo[d]imidazol-2-yl)-5-(3-fluoro-5-methylphenyl)pyridin-2-yl)amino)ethyl)amino)-3-oxopropoxy)ethyl)amino)-4-oxobutanoic acid (400 mg, 433 μmol, 84.9%); (M+H)⁺=924.5.

Step D-5, preparation of (2S,3R,4S,5S,6S)-2-(2-((2S,5S,9R)-9-((tert-butoxycarbonyl)amino)-20-((4-(4-((tert-butoxycarbonyl)amino)piperidin-1-yl)-3-(6-chloro-1H-benzo[d]imidazol-2-yl)-5-(3-fluoro-5-methylphenyl)pyridin-2-yl)amino)-5-isopropyl-4,7,10,17-tetraoxo-2-(3-ureidopropyl)-14-oxa-3,6,11,18-tetraazaicosanamido)-5-((5S,8S,11S,12R)-11-((S)-sec-butyl)-12-(2-((S)-2-((1R,2R)-3-(((1S,2R)-1-hydroxy-1-phenylpropan-2-yl)amino)-1-methoxy-2-methyl-3-oxopropyl)pyprrolidin-1-yl)-2-oxoethyl)-5,8-diisopropyl-4,10-dimethyl-3,6,9-trioxo-2,13-dioxa-4,7,10-triazatetradecyl)phenoxy)-6-(methoxycarbonyl)tetrahydro-2H-pyran-3,4,5-triyl triacetate: Into a 8-mL vial was placed (R)-3-((tert-butoxycarbonyl)amino)-4-((2-(3-((2-((4-(4-((tert-butoxycarbonyl)amino)piperidin-1-yl)-3-(6-chloro-1H-benzo[d]imidazol-2-yl)-5-(3-fluoro-5-methylphenyl)pyridin-2-yl)amino)ethyl)amino)-3-oxopropoxy)ethyl)amino)-4-oxobutanoic acid 100 mg, 1.26 Eq. 108 mol), pentafluorophenyldiphenylphosphinate (100 mg, 3.03 Eq, 260 μmol), 4-methylmorpholine (26 mg, 3.0 Eq, 0.26 mmol), and DMF (2 mL). The resulting reaction mixture was stirred for 5 minutes at 25° C. Then to this was added (2S,3R,4S,5S,6S)-2-(2-((S)-2-((S)-2-amino-3-methylbutanamido)-5-ureidopentanamido)-5-((5S,8S,11S,12R)-11-((S)-sec-butyl)-12-(2-((S)-2-((1R,2R)-3-(((1S,2R)-1-hydroxy-1-phenylpropan-2-yl)amino)-1-methoxy-2-methyl-3-oxopropyl)pyrrolidin-1-yl)-2-oxoethyl)-5,8-diisopropyl-4,10-dimethyl-3,6,9-trioxo-2,13-dioxa-4,7,10-triazatetradecyl)phenoxy)-6-(methoxycarbonyl)tetrahydro-2H-pyran-3,4,5-triyl triacetate (125 mg, 1 Eq, 85.9 μmol). The resulting reaction mixture was stirred for 1 hour at 25° C. The crude product was purified by Prep-HPLC with the following conditions: Column: SunFire prep OBD 19*150 mm 5 um; Mobile Phase A: Water (0.05% TFA); Mobile Phase B: ACN; Gradient: 25% B to 65% B in 8 min; Flow rate:50 mL/min; Wave Length: 220 nm. The collected fractions were dried by lyophilization to provide (2S,3R,4S,5S,6S)-2-(2-((2S,5S,9R)-9-((tert-butoxycarbonyl)amino)-20-((4-(4-((tert-butoxycarbonyl)amino)piperidin-1-yl)-3-(6-chloro-1H-benzo[d]imidazol-2-yl)-5-(3-fluoro-5-methylphenyl)pyridin-2-yl)amino)-5-isopropyl-4,7,10,17-tetraoxo-2-(3-ureidopropyl)-14-oxa-3,6,11,18-tetraazaicosanamido)-5-((5S,8S,11S,12R)-11-((S)-sec-butyl)-12-(2-((S)-2-((1R,2R)-3-(((1S,2R)-1-hydroxy-1-phenylpropan-2-yl)amino)-1-methoxy-2-methyl-3-oxopropyl)pyrrolidin-1-yl)-2-oxoethyl)-5,8-diisopropyl-4,10-dimethyl-3,6,9-trioxo-2,13-dioxa-4,7,10-triazatetradecyl)phenoxy)-6-(methoxy carbonyl)tetrahydro-2H-pyran-3,4,5-triyl triacetate (95 mg, 40 μmol, 47%); (M+H)⁺=1182.3.

Step D-6, preparation of (2S,3S,4S,5R,6S)-6-(2-((2S,5S,9R)-9-((tert-butoxycarbonyl)amino)-20-((4-(4-((tert-butoxycarbonyl)amino)piperidin-1-yl)-3-(6-chloro-1H-benzo[d]imidazol-2-yl)-5-(3-fluoro-5-methylphenyl)pyridin-2-v)amino)-5-isopropyl-4,7,10,17-tetraoxo-2-(3-ureidopropyl)-14-oxa-3,6,11,18-tetraazaicosanamido)-5-((5S,8S,11S,12R)-11-((S)-sec-butyl)-12-(2-((S)-2-((1R,2R)-3-(((I S,2R)-1-hydroxy-1-phenylpropan-2-yl)amino)-1-methoxy-2-methyl-3-oxopropyl)pyrrolidin-1-yl)-2-oxoethyl)-5,8-diisopropyl-4,10-dimethyl-3,6,9-trioxo-2,13-dioxa-4,7,10-triazatetradecyl)phenoxy)-3,4,5-trihydroxytetrahydro-2H-pyran-2-carboxylic acid: Into a 8-mL vial was placed a mixture of (2S,3R,4S,5S,6S)-2-(2-((2S,5S,9R)-9-((tert-butoxycarbonyl)amino)-20-((4-(4-((tert-butoxycarbonyl)amino)piperidin-1-yl)-3-(6-chloro-1H-benzo[d]imidazol-2-yl)-5-(3-fluoro-5-methylphenyl)pyridin-2-yl)amino)-5-isopropyl-4,7,10,17-tetraoxo-2-(3-ureidopropyl)-14-oxa-3,6,11,18-tetraazaicosanamido)-5-((5S,8S,11S,12R)-11-((S)-sec-butyl)-12-(2-((S)-2-((1R,2R)-3-(((I S,2R)-1-hydroxy-1-phenylpropan-2-yl)amino)-1-methoxy-2-methyl-3-oxopropyl)pyrrolidin-1-yl)-2-oxoethyl)-5,8-diisopropyl-4,10-dimethyl-3,6,9-trioxo-2,13-dioxa-4,7,10-triazatetradecyl)phenoxy)-6-(methoxycarbonyl)tetrahydro-2H-pyran-3,4,5-triyl triacetate (95 mg, 1 Eq, 40 μmol), lithium hydroxide (5 mg, 5 Eq, 0.2 mmol), MeOH (2 mL) and H₂O (1 mL). The reaction mixture was stirred at 0° C. for 1 hour. The mixture was directly purified by MPLC with the following conditions: Column, WelFlash™, C18120 g. Spherical 20-40 μm; Mobile phase, Water (0.1% TFA) and ACN (5% ACN to 5% ACN in 1 min, 30% ACN up to 80% in 8 min, 95% ACN to 95% in 3 min); Total flow rate, 70 mL/min; Detector, UV 220 nm. This provided (2S,3S,4S,5R,6S)-6-(2-((2S,5 S,9R)-9-((tert-butoxy carbonyl)amino)-20-((4-(4-((tert-butoxycarbonyl)amino)piperidin-1-yl)-3-(6-chloro-1H-benzo[d]imidazol-2-yl)-5-(3-fluoro-5-methylphenyl)pyridin-2-yl)amino)-5-isopropyl-4,7,10,17-tetraoxo-2-(3-ureidopropyl)-14-oxa-3,6,11,18-tetraazaicosanamido)-5-((5S,8S,11S,12R)-11-((S)-sec-butyl)-12-(2-((S)-2-((1R,2R)-3-(((1S,2R)-1-hydroxy-1-phenylpropan-2-yl)amino)-1-methoxy-2-methyl-3-oxopropyl)pyrrolidin-1-yl)-2-oxoethyl)-5,8-diisopropyl-4,10-dimethyl-3,6,9-trioxo-2,13-dioxa-4,7,10-triazatetradecyl)phenoxy)-3,4,5-trihydroxytetrahydro-2H-pyran-2-carboxylic acid (70 mg, 32 μmol, 78%); (M+H)⁺=1111.7.

Step D-7, preparation of (2S,3S,4S,5R,6S)-6-[15-({[(S)-1-[N—(S)-1-{1(1S,2R)-4-[(S)-2-{(1R,2R)-2-[N-(1R,2S)-2-hydroxy-1-methyl-2-phenylethylcarbamoyl]-1-methoxy propyl}-1-pyrrolidinyl]-1-[(S)-1-methylpropyl]-2-methoxy-4-oxobutyl]-N-methylcarbamoyl}-2-methylpropylcarbamoyl]-2-methylpropyl](methyl)(aminocarbonyloxy)}methyl)-2-[(S)-2-[(S)-2-[(R)-3-[N-2-(2-{N-2-[4-(4-amino-1-piperidyl)-3-(6-chloro-1H-1,3-benzimidazol-2-yl)-5-(3-fluoro-5-tolyl)-2-pyridylamino]ethylcarbamoyl}ethoxy)ethylcarbamoyl]-3-aminopropionylamino]-3-methylbutyrylamino]-5-ureidovalerylamino]phenoxyl-3,4,5-trihydroxytetrahydro-2H-pyran-2-carboxylic acid: Into a 8 mL vial was placed a mixture of (2S,3S,4S,5R,6S)-6-(2-((2S,5S,9R)-9-((tert-butoxycarbonyl)amino)-20-((4-(4-((tert-butoxy carbonyl)amino)piperidin-1-yl)-3-(6-chloro-1H-benzo[d]imidazol-2-yl)-5-(3-fluoro-5-methylphenyl)pyridin-2-yl)amino)-5-isopropyl-4,7,10,17-tetraoxo-2-(3-ureidopropyl)-14-oxa-3,6,11,18-tetraazaicosanamido)-5-((5S,8S,11S,12R)-11-((S)-sec-butyl)-12-(2-((S)-2-((1R,2R)-3-(((1S,2R)-1-hydroxy-1-phenylpropan-2-yl)amino)-1-methoxy-2-methyl-3-oxopropyl)pyrrolidin-1-yl)-2-oxoethyl)-5,8-diisopropyl-4,10-dimethyl-3,6,9-trioxo-2,13-dioxa-4,7,10-triazatetradecyl)phenoxy)-3,4,5-trihydroxytetrahydro-2H-pyran-2-carboxylic acid (70 mg, 1 Eq, 32 μmol), DCM (0.6 mL) and TFA (0.3 mL), and the reaction mixture was stirred at 0° C. for 30 minutes. The crude product was purified by Prep-HPLC with the following conditions: Column: SunFire prep OBD 19*150 mm 5 um; Mobile Phase A: Water (0.05% TFA); Mobile Phase B: ACN: Gradient: 25% B to 65% B in 8 min; Flow rate: 60 mL/min; Wavelength: 220 nm. The collected fractions were dried by lyophilization to provide (2S,3S,4S,5R,6S)-6-[5-({[(S)-1-[N—(S)-1-{[(1S,2R)-4-[(S)-2-{(1R,2R)-2-[N-(1R,2S)-2-hydroxy-1-methyl-2-phenylethylcarbamoyl]-1-methoxypropyl}-1-pyrrolidinyl]-1-[(S)-1-methylpropyl]-2-methoxy-4-oxobutyl]-N-methylcarbamoyl}-2-methylpropylcarbamoyl]-2-methylpropyl](methyl)(aminocarbonyloxy)}methyl)-2-[(S)-2-[(S)-2-[(R)-3-[N-2-(2-{N-2-[4-(4-amino-1-piperidyl)-3-(6-chloro-1H-1,3-benzimidazol-2-yl)-5-(3-fluoro-5-tolyl)-2-pyridylamino]ethylcarbamoyl}ethoxy)ethylcarbamoyl]-3-aminopropionylamino]-3-methylbutyrylamino]-5-ureidovalerylamino]phenoxy]-3,4,5-trihydroxytetrahydro-2H-pyran-2-carboxylic acid (41.7 mg, 17.5 μmol, 56%, 99.3% Purity); (M+H)⁺=2021.0.

The following conjugates were prepared similarly to Example D with appropriate substituting reagents and substrates at different steps and they may require additional functional group modifications via well-known chemistry with appropriate reagents

| | MS |
|---|---|
| | (M + |
| Compound | H)⁺ |
| (2S,3S,4S,5R,6S)-6-[5-({[(S)-1-[N-(S)-1-{[(1S,2R)-4-[(S)-2-{(1R,2R)-2-[N-(1R,2S)-2-hydroxy-1-methyl-2-phenylethylcarbamoyl]-1- | 1932.0 |
| methoxypropyl}-1-pyrrolidinyl]-1-[(S)-1-methylpropyl]-2-methoxy-4-oxobutyl]-N-methylcarbamoyl}-2-methylpropylcarbamoyl]-2- | |
| methylpropyl](methyl)(aminocarbonyloxy)}methyl)-2-[(S)-2-[(S)-2-{3-[2-(4-{N-2-[4-(4-amino-1-piperidyl)-3-(6-chloro-1H-1,3- | |
| benzimidazol-2-yl)-5-(3-fluoro-5-tolyl)-2-pyridylamino]ethylcarbamoyl}-1-piperidyl)ethoxy]propionylamino}-3- | |
| methylbutyrylamino]propionylamino]phenoxy]-3,4,5-trihydroxytetrahydro-2H-pyran-2-carboxylic acid | |
| 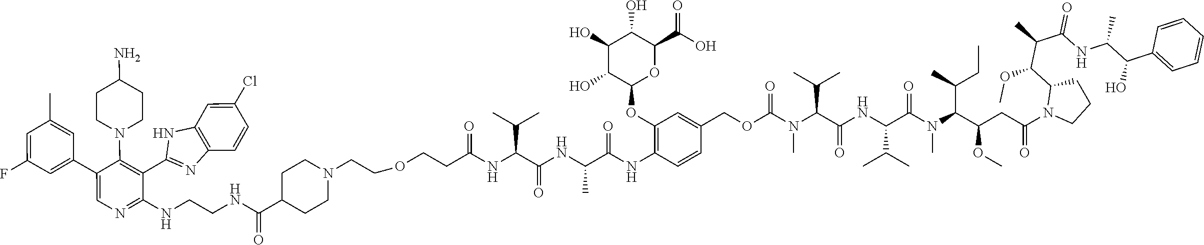 [SMILES: CC[C@H](C)[C@@H]([C@@H](CC(=O)N1CCC[C@H]1[C@H](OC)[C@@H](C)C(=O)N[C@H](C)[C@@H](O)c1ccccc1)OC)N(C)C(=O)[C@@H](NC(=O)[C@H](C(C)C)N(C)C(=O)OCc1ccc(NC(=O)[C@H](C)NC(=O)[C@@H](NC(=O)CCOCCN2CCC(C(=O)NCCNc3ncc(-c4cc(C)cc(F)c4)c(N4CCC(N)CC4)c3-c3nc4ccc(Cl)cc4[nH]3)CC2)C(C)C)c(O[C@@H]2O[C@H](C(=O)O)[C@@H](O)[C@H](O)[C@H]2O)c1)C(C)C] | |
| (Compound 93) | |
| (2S,3S,4S,5R,6S)-6-[5-({[(S)-1-[N-(S)-1-{[(1S,2R)-4-[(S)-2-{(1R,2R)-2-[N-(1R,2S)-2-hydroxy-1-methyl-2-phenylethylcarbamoyl]-1- | 1949.0 |
| methoxypropyl}-1-pyrrolidinyl]-1-[(S)-1-methylpropyl]-2-methoxy-4-oxobutyl]-N-methylcarbamoyl}-2-methylpropylcarbamoyl]-2- | |
| methylpropyl](methyl)(aminocarbonyloxy)}methyl)-2-[(S)-2-[(S)-2-[3-(2-{N-2-[4-(4-amino-1-piperidyl)-3-(5-fluoro-7-methoxy-1H- | |
| 1,3-benzimidazol-2-yl)-5-(3-fluoro-5-tolyl)-2-pyridylamino]ethylcarbamoyl}ethoxy)propionylamino]-3-methylbutyrylamino]-5- | |
| ureidovalerylamino]phenoxy]-3,4,5-trihydroxytetrahydro-2H-pyran-2-carboxylic acid | |
| 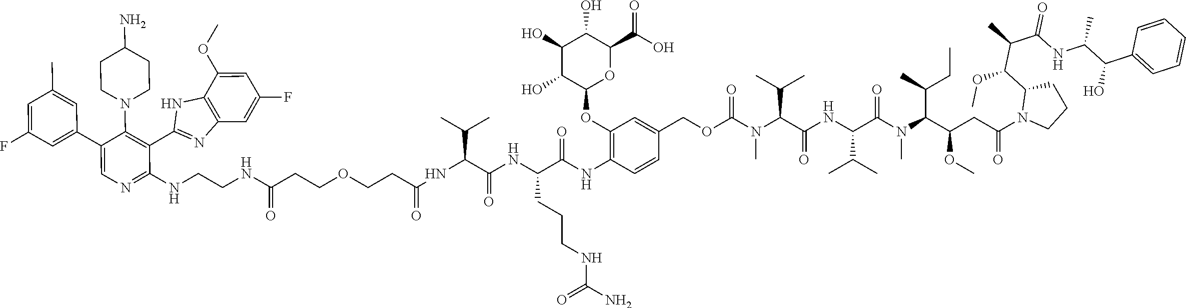 [SMILES: CC[C@H](C)[C@@H]([C@@H](CC(=O)N1CCC[C@H]1[C@H](OC)[C@@H](C)C(=O)N[C@H](C)[C@@H](O)c1ccccc1)OC)N(C)C(=O)[C@@H](NC(=O)[C@H](C(C)C)N(C)C(=O)OCc1ccc(NC(=O)[C@H](CCCNC(N)=O)NC(=O)[C@@H](NC(=O)CCOCCC(=O)NCCNc2ncc(-c3cc(C)cc(F)c3)c(N3CCC(N)CC3)c2-c2nc3cc(F)cc(OC)c3[nH]2)C(C)C)c(O[C@@H]2O[C@H](C(=O)O)[C@@H](O)[C@H](O)[C@H]2O)c1)C(C)C] | |
| (Compound 94) | |
| (2S,3S,4S,5R,6S)-6-[5-({[(S)-1-[N-(S)-1-{[(1S,2R)-4-[(S)-2-{(1R,2R)-2-[N-(1R,2S)-2-hydroxy-1-methyl-2-phenylethylcarbamoyl]-1- | 1850.9 |
| methoxypropyl}-1-pyrrolidinyl]-1-[(S)-1-methylpropyl]-2-methoxy-4-oxobutyl]-N-methylcarbamoyl}-2-methylpropylcarbamoyl]-2- | |
| methylpropyl](methyl)(aminocarbonyloxy)}methyl)-2-[(S)-2-[(S)-2-[3-(2-{N-2-[4-(4-amino-1-piperidyl)-3-(5,7-difluoro-1H-1,3- | |
| benzimidazol-2-yl)-5-(3-fluoro-5-tolyl)-2-pyridylamino]ethylcarbamoyl}ethoxy)propionylamino]-3- | |
| methylbutyrylamino]propionylamino]phenoxy]-3,4,5-trihydroxytetrahydro-2H-pyran-2-carboxylic acid | |
| 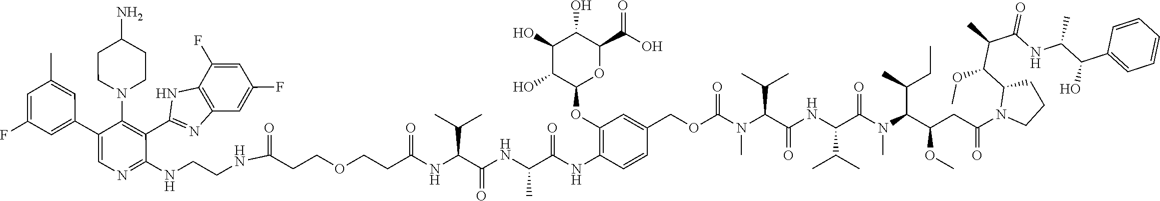 [SMILES: CC[C@H](C)[C@@H]([C@@H](CC(=O)N1CCC[C@H]1[C@H](OC)[C@@H](C)C(=O)N[C@H](C)[C@@H](O)c1ccccc1)OC)N(C)C(=O)[C@@H](NC(=O)[C@H](C(C)C)N(C)C(=O)OCc1ccc(NC(=O)[C@H](C)NC(=O)[C@@H](NC(=O)CCOCCC(=O)NCCNc2ncc(-c3cc(C)cc(F)c3)c(N3CCC(N)CC3)c2-c2nc3cc(F)cc(F)c3[nH]2)C(C)C)c(O[C@@H]2O[C@H](C(=O)O)[C@@H](O)[C@H](O)[C@H]2O)c1)C(C)C] | |
| (Compound 95) | |
| (2S,3S,4S,5R,6S)-6-[5-({[(S)-1-[N-(S)-1-{[(1S,2R)-4-[(S)-2-{(1R,2R)-2-[N-(1R,2S)-2-hydroxy-1-methyl-2-phenylethylcarbamoyl]-1- | 1839.9 |
| methoxypropyl-1-pyrrolidinyl]-1-[(S)-1-methylpropyl]-2-methoxy-4-oxobutyl]-N-methylcarbamoyl}-2-methylpropylcarbamoyl]-2- | |
| methylpropyl](methyl)(aminocarbonyloxy)}methyl)-2-[(S)-2-[(S)-2-[3-(2-{N-2-[4-(4-amino-1-piperidy)-3-(6-cyano-1H-1,3- | |
| benzimidazol-2-yl)-5-(3-fluoro-5-tolyl)-2-pyridylamino]ethylcarbamoyl}ethoxy)propionylamino]-3- | |
| methylbutyrylamino]propionylamino]phenoxy]-3,4,5-trihydroxytetrahydro-2H-pyran-2-carboxylic acid | |
| 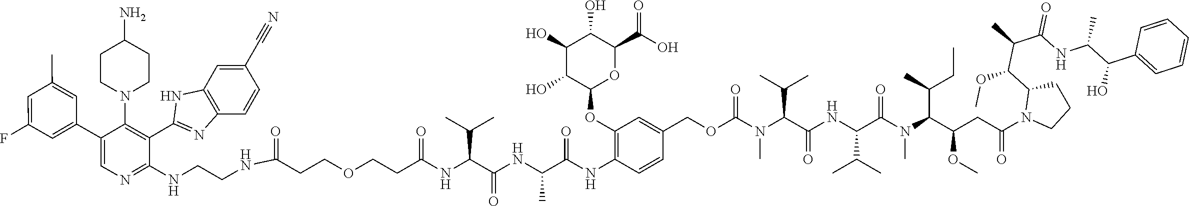 [SMILES: CC[C@H](C)[C@@H]([C@@H](CC(=O)N1CCC[C@H]1[C@H](OC)[C@@H](C)C(=O)N[C@H](C)[C@@H](O)c1ccccc1)OC)N(C)C(=O)[C@@H](NC(=O)[C@H](C(C)C)N(C)C(=O)OCc1ccc(NC(=O)[C@H](C)NC(=O)[C@@H](NC(=O)CCOCCC(=O)NCCNc2ncc(-c3cc(C)cc(F)c3)c(N3CCC(N)CC3)c2-c2nc3ccc(C#N)cc3[nH]2)C(C)C)c(O[C@@H]2O[C@H](C(=O)O)[C@@H](O)[C@H](O)[C@H]2O)c1)C(C)C] | |
| (Compound 96) | |
| (2S,3S,4S,5R,6S)-6-[5-({[(S)-1-[N-(S)-1-{[(1S,2R)-4-[(S)-2-{(1R,2R)-2-[N-(1R,2S)-2-hydroxy-1-methyl-2-phenylethylcarbamoyl]-1- | 1844.9 |
| methoxypropyl}-1-pyrrolidinyl]-1-[(S)-1-methylpropyl]-2-methoxy-4-oxobutyl]-N-methylcarbamoyl}-2-methylpropylcarbamoyl]-2- | |
| methylpropyl](methyl)(aminocarbonyloxy)}methyl)-2-[(S)-2-[(S)-2-[3-(2-{N-2-[4-(4-amino-1-piperidyl)-5-(3-fluoro-5-tolyl)-3-(5- | |
| methoxy-1H-1,3-benzimidazol-2-yl)-2-pyridylamino]ethylcarbamoyl}ethoxy)propionylamino]-3- | |
| methylbutyrylamino]propionylamino]phenoxy]-3,4,5-trihydroxytetrahydro-2H-pyran-2-carboxylic acid | |
| 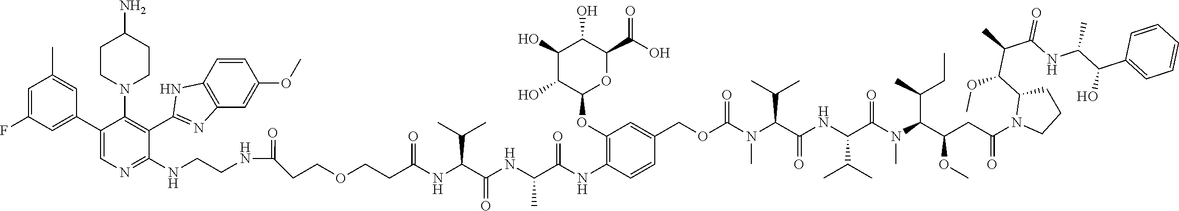 [SMILES: CC[C@H](C)[C@@H]([C@@H](CC(=O)N1CCC[C@H]1[C@H](OC)[C@@H](C)C(=O)N[C@H](C)[C@@H](O)c1ccccc1)OC)N(C)C(=O)[C@@H](NC(=O)[C@H](C(C)C)N(C)C(=O)OCc1ccc(NC(=O)[C@H](C)NC(=O)[C@@H](NC(=O)CCOCCC(=O)NCCNc2ncc(-c3cc(C)cc(F)c3)c(N3CCC(N)CC3)c2-c2nc3cc(OC)ccc3[nH]2)C(C)C)c(O[C@@H]2O[C@H](C(=O)O)[C@@H](O)[C@H](O)[C@H]2O)c1)C(C)C] | |
| (Compound 97) | |
| (2S,3S,4S,5R,6S)-6-[5-({[(S)-1-[N-(S)-1-{[(1S,2R)-4-[(S)-2-{(1R,2R)-2-[N-(1R,2S)-2-hydroxy-1-methyl-2-phenylethylcarbamoy]-1- | 1869.9 |
| methoxypropyl}-1-pyrrolidinyl]-1-[(S)-1-methylpropyl]-2-methoxy-4-oxobutyl]-N-methylcarbamoyl}-2-methylpropylcarbamoy]-2- | |
| methylpropyl](methyl)(aminocarbonyloxy)}methyl)-2-[(S)-2-[(S)-2-[3-(2-{N-2-[4-(4-amino-1-piperidyl)-3-(6-cyano-4-methoxy-1H- | |
| 1,3-benzimidazol-2-yl)-5-(3-fluoro-5-tolyl)-2-pyridylamino]ethylcarbamoyl}ethoxy )propionylamino]-3- | |
| methylbutyrylamino]propionylamino]phenoxy]-3,4,5-trihydroxytetrahydro-2H-pyran-2-carboxylic acid | |
| 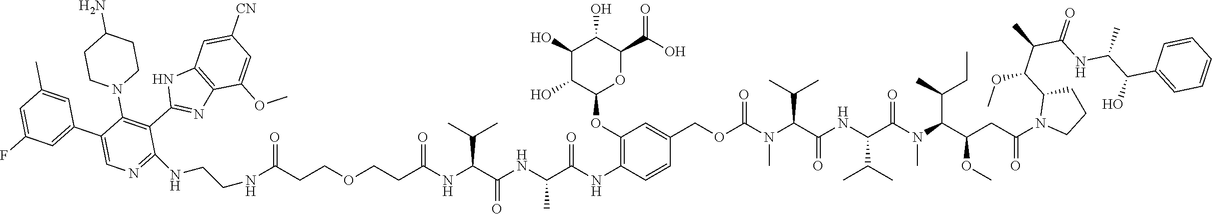 [SMILES: CC[C@H](C)[C@@H]([C@@H](CC(=O)N1CCC[C@H]1[C@H](OC)[C@@H](C)C(=O)N[C@H](C)[C@@H](O)c1ccccc1)OC)N(C)C(=O)[C@@H](NC(=O)[C@H](C(C)C)N(C)C(=O)OCc1ccc(NC(=O)[C@H](C)NC(=O)[C@@H](NC(=O)CCOCCC(=O)NCCNc2ncc(-c3cc(C)cc(F)c3)c(N3CCC(N)CC3)c2-c2nc3c(OC)cc(C#N)cc3[nH]2)C(C)C)c(O[C@@H]2O[C@H](C(=O)O)[C@@H](O)[C@H](O)[C@H]2O)c1)C(C)C] | |
| (Compound 98) | |
| (2S,3S,4S,5R,6S)-6-[5-({[(S)-1-[N-(S)-1-{[(1S,2R)-4-[(S)-2-{(1R,2R)-2-[N-(1R,2S)-2-hydroxy-1-methyl-2-phenylethylcarbamoyl]-1- | 1857.9 |
| methoxypropyl}-1-pyrrolidiny1]-1-[(S)-1-methylpropyl]-2-methoxy-4-oxobutyl]-N-methylcarbamoyl}-2-methylpropylcarbamoyl]-2- | |
| methylpropyl](methyl)(aminocarbonyloxy)}methy1)-2-[(S)-2-[(S)-2-[3-(2-{N-2-[4-(4-amino-1-piperidyl)-3-(6-cyano-4-fluoro-1H-1,3- | |
| benzimidazol-2-yl)-5-(3-fluoro-5-tolyl)-2-pyridylamino]ethylcarbamoyl}ethoxy )propionylamino]-3- | |
| methylbutyrylamino]propionylamino]phenoxy ]-3,4,5-trihydroxytetrahydro-2H-pyran-2-carboxylic acid | |
| 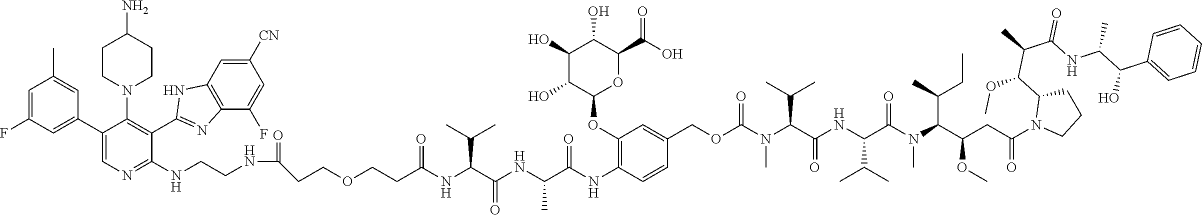 [SMILES: CC[C@H](C)[C@@H]([C@@H](CC(=O)N1CCC[C@H]1[C@H](OC)[C@@H](C)C(=O)N[C@H](C)[C@@H](O)c1ccccc1)OC)N(C)C(=O)[C@@H](NC(=O)[C@H](C(C)C)N(C)C(=O)OCc1ccc(NC(=O)[C@H](C)NC(=O)[C@@H](NC(=O)CCOCCC(=O)NCCNc2ncc(-c3cc(C)cc(F)c3)c(N3CCC(N)CC3)c2-c2nc3c(F)cc(C#N)cc3[nH]2)C(C)C)c(O[C@@H]2O[C@H](C(=O)O)[C@@H](O)[C@H](O)[C@H]2O)c1)C(C)C] | |
| (Compound 99) | |
| (2S,3S,4S,5R,6S)-6-[5-({[(S)-1 -[N-(S)-1-{[(1S,2R)-4-[(S)-2-{(1R,2R)-2-[N-(1R,2S)-2-hydroxy-1-methyl-2-phenylethylcarbamoyl]-1- | 1866.9 |
| methoxypropyl}-1-pyrrolidinyl]-1-[(S)-1-methylpropyl]-2-methoxy-4-oxobutyl]-N-methylcarbamoyl}-2-methylpropylcarbamoyl]-2- | |
| methylpropyl](methyl)(aminocarbonyloxy)}methyl)-2-[(S)-2-[(S)-2-[3-(2-{N-2-[4-(4-amino-1-piperidyl)-3-(5,7-difluoro-1H-1,3- | |
| benzimidazol-2-yl)-5-(3-fluoro-5-methoxyphenyl)-2-pyridylamino]ethylcarbamoyl}ethoxy)propionylamino]-3- | |
| methylbutyrylamino]propionylamino]phenoxy]-3,4,5-trihydroxytetrahydro-2H-pyran-2-carboxylic acid | |
| 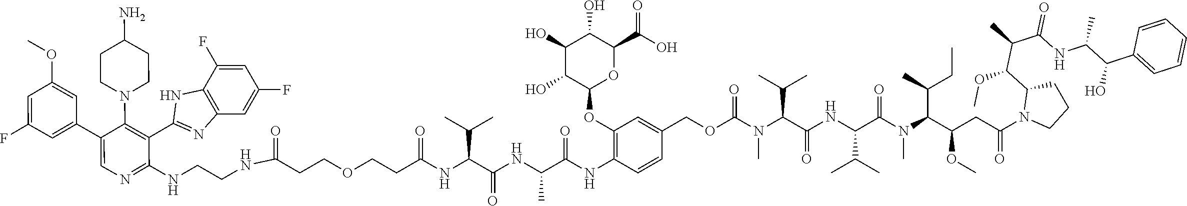 [SMILES: CC[C@H](C)[C@@H]([C@@H](CC(=O)N1CCC[C@H]1[C@H](OC)[C@@H](C)C(=O)N[C@H](C)[C@@H](O)c1ccccc1)OC)N(C)C(=O)[C@@H](NC(=O)[C@H](C(C)C)N(C)C(=O)OCc1ccc(NC(=O)[C@H](C)NC(=O)[C@@H](NC(=O)CCOCCC(=O)NCCNc2ncc(-c3cc(F)cc(OC)c3)c(N3CCC(N)CC3)c2-c2nc3cc(F)cc(F)c3[nH]2)C(C)C)c(O[C@@H]2O[C@H](C(=O)O)[C@@H](O)[C@H](O)[C@H]2O)c1)C(C)C] | |
| (Compound 100) | |
| (2S,3S,4S,5R,6S)-6-[5-({[(S)-1-[N-(S)-1-{[(1S,2R)-4-[(S)-2-{(1R,2R)-2-[N-(1R,2S)-2-hydroxy-1-methyl-2-phenylethylcarbamoyl]-1- | 1937.0 |
| methoxypropyl}-1-pyrrolidinyl]-1-[(S)-1-methylpropyl]-2-methoxy-4-oxobutyl]-N-methylcarbamoyl}-2-methylpropylcarbamoyl]-2- | |
| methylpropyl](methyl)(aminocarbonyloxy)}methyl)-2-[(S)-2-[(S)-2-[3-(2-{N-2-[4-(4-amino-1-piperidyl)-3-(5,7-difluoro-1H-1,3- | |
| benzimidazol-2-yl)-5-(3-fluoro-5-tolyl)-2-pyridylamino]ethylcarbamoyl}ethoxy)propionylamino]-3-methylbutyrylamino]-5- | |
| ureidovalerylamino]phenoxy]-3,4,5-trihydroxytetrahydro-2H-pyran-2-carboxylic acid | |
| 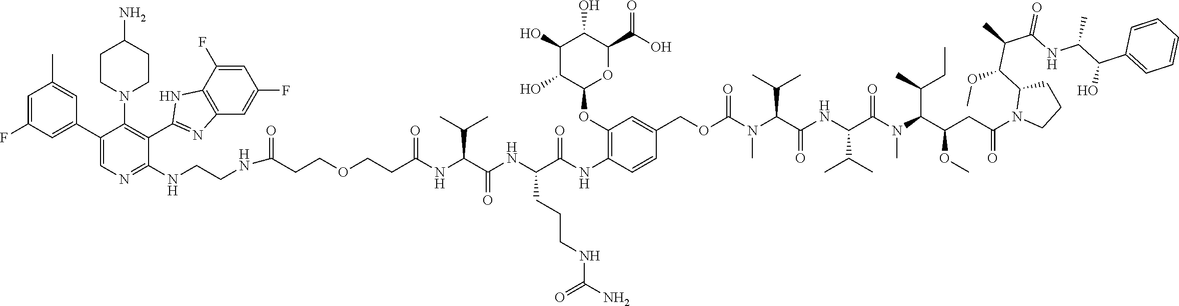 [SMILES: CC[C@H](C)[C@@H]([C@@H](CC(=O)N1CCC[C@H]1[C@H](OC)[C@@H](C)C(=O)N[C@H](C)[C@@H](O)c1ccccc1)OC)N(C)C(=O)[C@@H](NC(=O)[C@H](C(C)C)N(C)C(=O)OCc1ccc(NC(=O)[C@H](CCCNC(N)=O)NC(=O)[C@@H](NC(=O)CCOCCC(=O)NCCNc2ncc(-c3cc(C)cc(F)c3)c(N3CCC(N)CC3)c2-c2nc3cc(F)cc(F)c3[nH]2)C(C)C)c(O[C@@H]2O[C@H](C(=O)O)[C@@H](O)[C@H](O)[C@H]2O)c1)C(C)C] | |
| (Compound 101) | |
| (2S,3S,4S,5R,6S)-6-[5-({[(S)-1-[N-(S)-1-{[(1S,2R)-4-[(S)-2-{(1R,2R)-2-[N-(1R,2S)-2-hydroxy-1-methyl-2-phenylethylcarbamoyl]-1- | 1937.0 |
| methoxypropyl}-1-pyrrolidinyl]-1-[(S)-1-methylpropyl]-2-methoxy-4-oxobutyl]-N-methylcarbamoyl}-2-methylpropylcarbamoyl]-2- | |
| methylpropyl](methyl)(aminocarbonyloxy)}methyl)-2-[(S)-2-[(S)-2-[3-(2-{N-2-[4-(4-amino-1-piperidyl)-3-(5,6-difluoro-1H-1,3- | |
| benzimidazol-2-yl)-5-(3-fluoro-5-tolyl)-2-pyridylamino]ethylcarbamoyl}ethoxy)propionylamino]-3-methylbutyrylamino]-5- | |
| ureidovalerylamino phenoxy]-3,4,5-trihydroxytetrahydro-2H-pyran-2-carboxylic acid | |
| 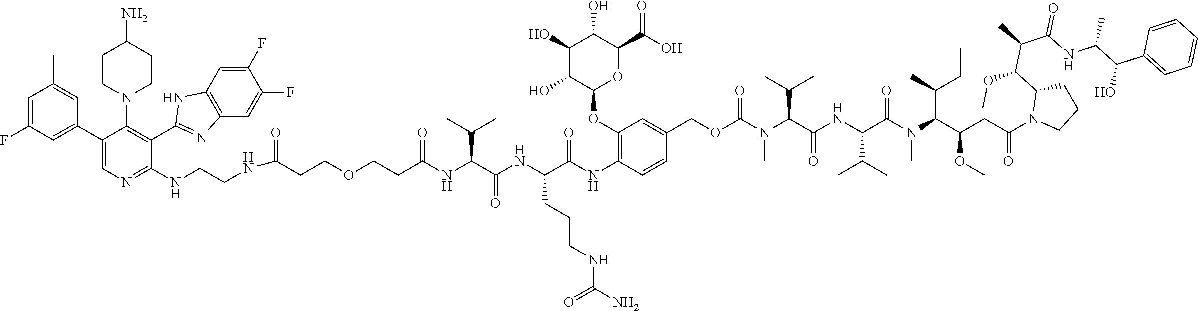 [SMILES: CC[C@H](C)[C@@H]([C@@H](CC(=O)N1CCC[C@H]1[C@H](OC)[C@@H](C)C(=O)N[C@H](C)[C@@H](O)c1ccccc1)OC)N(C)C(=O)[C@@H](NC(=O)[C@H](C(C)C)N(C)C(=O)OCc1ccc(NC(=O)[C@H](CCCNC(N)=O)NC(=O)[C@@H](NC(=O)CCOCCC(=O)NCCNc2ncc(-c3cc(C)cc(F)c3)c(N3CCC(N)CC3)c2-c2nc3cc(F)c(F)cc3[nH]2)C(C)C)c(O[C@@H]2O[C@H](C(=O)O)[C@@H](O)[C@H](O)[C@H]2O)c1)C(C)C] | |
| (Compound 102) | |
| (2S,3S,4S,5R,6S)-6-[5-({[(S)-1-[N-(S)-1-{[(1S,2R)-4-[(S)-2-{(1R,2R)-2-[N-(1R,2S)-2-hydroxy-1-methyl-2-phenylethylcarbamoyl]-1- | 1925.7 |
| methoxypropyl}-1-pyrrolidinyl]-1-[(S)-1-methylpropyl]-2-methoxy-4-oxobutyl]-N-methylcarbamoyl}-2-methylpropylcarbamoyl]-2- | |
| methylpropyl](methyl)(aminocarbonyloxy)}methyl)-2-[(S)-2-[(S)-2-[3-(2-{N-2-[4-(4-amino-1-piperidyl)-3-(6-cyano-1H-1,3- | |
| benzimidazol-2-yl)-5-(3-fluoro-5-tolyl)-2-pyridylamino]ethylcarbamoyl}ethoxy)propionylamino]-3-methylbutyrylamino]-5- | |
| ureidovalerylamino phenoxy]-3,4,5-trihydroxytetrahydro-2H-pyran-2-carboxylic acid | |
| 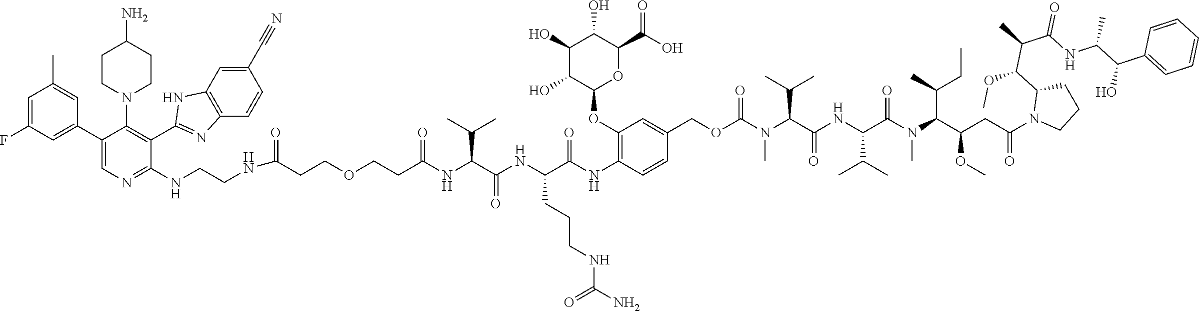 [SMILES: CC[C@H](C)[C@@H]([C@@H](CC(=O)N1CCC[C@H]1[C@H](OC)[C@@H](C)C(=O)N[C@H](C)[C@@H](O)c1ccccc1)OC)N(C)C(=O)[C@@H](NC(=O)[C@H](C(C)C)N(C)C(=O)OCc1ccc(NC(=O)[C@H](CCCNC(N)=O)NC(=O)[C@@H](NC(=O)CCOCCC(=O)NCCNc2ncc(-c3cc(C)cc(F)c3)c(N3CCC(N)CC3)c2-c2nc3ccc(C#N)cc3[nH]2)C(C)C)c(O[C@@H]2O[C@H](C(=O)O)[C@@H](O)[C@H](O)[C@H]2O)c1)C(C)C] | |
| (Compound 103) | |
| (2S,3S,4S,5R,6S)-6-[5-({[(S)-1-[N-(S)-1-{[(1S,2R)-4-[(S)-2-{(1R,2R)-2-[N-(1R,2S)-2-hydroxy-1-methyl-2-phenylethylcarbamoyl]-1- | 1956.0 |
| methoxypropyl}-1-pyrrolidinyl]-1-[(S)-1-methylpropyl]-2-methoxy-4-oxobutyl]-N-methylcarbamoyl}-2-methylpropylcarbamoyl]-2- | |
| methylpropyl](methyl)(aminocarbonyloxy)}methyl)-2-[(S)-2-[(S)-2-[3-(2-{N-2-[4-(4-amino-1-piperidyl)-3-(6-cyano-4-methoxy-1H- | |
| 1,3-benzimidazol-2-yl)-5-(3-fluoro-5-toly])-2-pyridylamino]ethylcarbamoyl}ethoxy)propionylamino]-3-methylbutyrylamino]-5- | |
| ureidovalerylamino]phenoxy]-3,4,5-trihydroxytetrahydro-2H-pyran-2-carboxylic acid | |
| 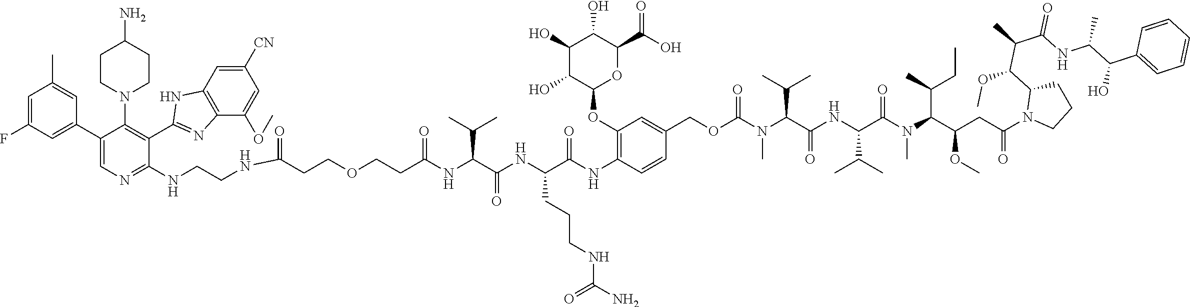 [SMILES: CC[C@H](C)[C@@H]([C@@H](CC(=O)N1CCC[C@H]1[C@H](OC)[C@@H](C)C(=O)N[C@H](C)[C@@H](O)c1ccccc1)OC)N(C)C(=O)[C@@H](NC(=O)[C@H](C(C)C)N(C)C(=O)OCc1ccc(NC(=O)[C@H](CCCNC(N)=O)NC(=O)[C@@H](NC(=O)CCOCCC(=O)NCCNc2ncc(-c3cc(C)cc(F)c3)c(N3CCC(N)CC3)c2-c2nc3c(OC)cc(C#N)cc3[nH]2)C(C)C)c(O[C@@H]2O[C@H](C(=O)O)[C@@H](O)[C@H](O)[C@H]2O)c1)C(C)C] | |
| (Compound 104) | |
| (2S,3S,4S,5R,6S)-6-[5-({[(S)-1-[N-(S)-1-{[(1S,2R)-4-[(S)-2-{(1R,2R)-2-[N-(1R,2S)-2-hydroxy-1-methyl-2-phenylethylcarbamoyl]-1- | 1931.0 |
| methoxypropyl}-1-pyrrolidinyl]-1-[(S)-1-methylpropyl]-2-methoxy-4-oxobutyl]-N-methylcarbamoyl}-2-methylpropylcarbamoyl]-2- | |
| methylpropyl](methyl)(aminocarbonyloxy)}methyl)-2-[(S)-2-[(S)-2-[3-(2-{N-2-[4-(4-amino-1-piperidyl)-5-(3-fluoro-5-tolyl)-3-(5- | |
| methoxy-1H-1,3-benzimidazol-2-yl)-2-pyridylamino]ethylcarbamoyl}ethoxy)propionylamino]-3-methylbutyrylamino]-5- | |
| ureidovalerylaminophenoxy]-3,4,5-trihydroxytetrahydro-2H-pyran-2-carboxylic acid | |
| 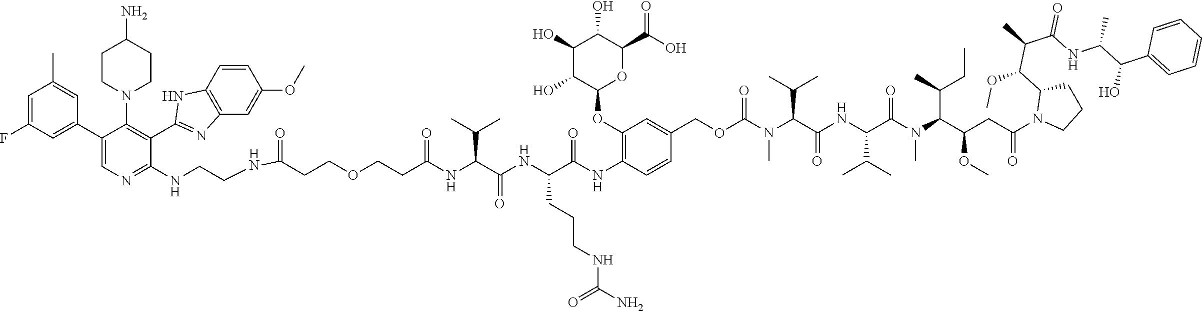 [SMILES: CC[C@H](C)[C@@H]([C@@H](CC(=O)N1CCC[C@H]1[C@H](OC)[C@@H](C)C(=O)N[C@H](C)[C@@H](O)c1ccccc1)OC)N(C)C(=O)[C@@H](NC(=O)[C@H](C(C)C)N(C)C(=O)OCc1ccc(NC(=O)[C@H](CCCNC(N)=O)NC(=O)[C@@H](NC(=O)CCOCCC(=O)NCCNc2ncc(-c3cc(C)cc(F)c3)c(N3CCC(N)CC3)c2-c2nc3cc(OC)ccc3[nH]2)C(C)C)c(O[C@@H]2O[C@H](C(=O)O)[C@@H](O)[C@H](O)[C@H]2O)c1)C(C)C] | |
| (Compound 105) | |
| (2S,3S,4S,5R,6S)-6-[5-({[(S)-1-[N-(S)-1-{[(1S,2R)-4-[(S)-2-{(1R,2R)-2-[N-(1R,2S)-2-hydroxy-1-methyl-2-phenylethylcarbamoyl]-1- | 1944.0 |
| methoxypropyl}-1-pyrrolidiny1]-1-[(S)-1-methylpropyl]-2-methoxy-4-oxobutyl]-N-methylcarbamoyl}-2-methylpropylcarbamoyl]-2- | |
| methylpropy](methyl)(aminocarbonyloxy)}methyl)-2-[(S)-2-[(S)-2-[3-(2-{N-2-[4-(4-amino-1-piperidyl)-3-(6-cyano-4-fluoro-1H-1,3- | |
| benzimidazol-2-yl)-5-(3-fluoro-5-tolyl)-2-pyridylamino]ethylcarbamoyl}ethoxy)propionylamino]-3-methylbutyrylamino]-5- | |
| ureidovalerylaminophenoxy]-3,4,5-trihydroxytetrahydro-2H-pyran-2-carboxylic acid | |
| 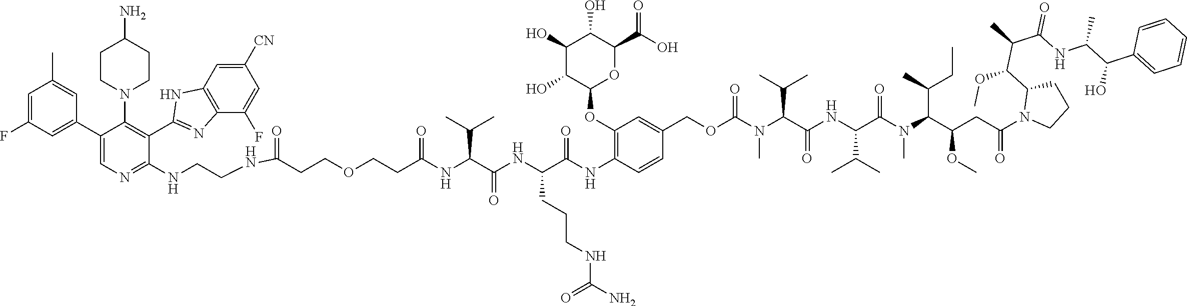 [SMILES: CC[C@H](C)[C@@H]([C@@H](CC(=O)N1CCC[C@H]1[C@H](OC)[C@@H](C)C(=O)N[C@H](C)[C@@H](O)c1ccccc1)OC)N(C)C(=O)[C@@H](NC(=O)[C@H](C(C)C)N(C)C(=O)OCc1ccc(NC(=O)[C@H](CCCNC(N)=O)NC(=O)[C@@H](NC(=O)CCOCCC(=O)NCCNc2ncc(-c3cc(C)cc(F)c3)c(N3CCC(N)CC3)c2-c2nc3c(F)cc(C#N)cc3[nH]2)C(C)C)c(O[C@@H]2O[C@H](C(=O)O)[C@@H](O)[C@H](O)[C@H]2O)c1)C(C)C] | |
| (Compound 106) | |
| (2S,3S,4S,5R,6S)-6-[5-({[(S)-1-[N-(S)-1-{[(1S,2R)-4-[(S)-2-{(1R,2R)-2-[N-(1R,2S)-2-hydroxy-1-methyl-2-phenylethylcarbamoyl]-1- | 1953.0 |
| methoxypropyl}-1-pyrrolidinyl]-1-[(S)-1-methylpropyl]-2-methoxy-4-oxobutyl]-N-methylcarbamoyl}-2-methylpropylcarbamoyl]-2- | |
| methylpropyl](methyl)(aminocarbonyloxy)}methyl)-2-[(S)-2-[(S)-2-[3-(2-{N-2-[4-(4-amino-1-piperidyl)-3-(5,7-difluoro-1H-1,3- | |
| benzimidazol-2-yl)-5-(3-fluoro-5-methoxyphenyl)-2-pyridylamino]ethylcarbamoyl}ethoxy)propionylamino]-3-methylbutyrylamino]-5- | |
| ureidovalerylamino phenoxy ]-3,4,5-trihydroxytetrahydro-2H-pyran-2-carboxylic acid | |
| 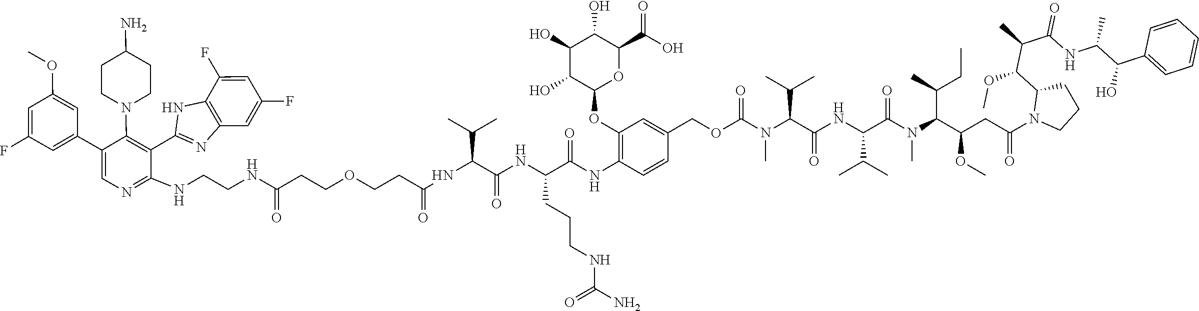 [SMILES: CC[C@H](C)[C@@H]([C@@H](CC(=O)N1CCC[C@H]1[C@H](OC)[C@@H](C)C(=O)N[C@H](C)[C@@H](O)c1ccccc1)OC)N(C)C(=O)[C@@H](NC(=O)[C@H](C(C)C)N(C)C(=O)OCc1ccc(NC(=O)[C@H](CCCNC(N)=O)NC(=O)[C@@H](NC(=O)CCOCCC(=O)NCCNc2ncc(-c3cc(F)cc(OC)c3)c(N3CCC(N)CC3)c2-c2nc3cc(F)cc(F)c3[nH]2)C(C)C)c(O[C@@H]2O[C@H](C(=O)O)[C@@H](O)[C@H](O)[C@H]2O)c1)C(C)C] | |
| (Compound 107) | |
| (2S,3S,4S,5R,6S)-6-[5-({[(S)-1-[N-(S)-1-{[(1S,2R)-4-[(S)-2-{(1R,2R)-2-[N-(1R,2S)-2-hydroxy-1-methyl-2-phenylethylcarbamoyl]-1- | 1850.8 |
| methoxypropyl}-1-pyrrolidinyl]-1-[(S)-1-methylpropyl]-2-methoxy-4-oxobutyl]-N-methylcarbamoyl}-2-methylpropylcarbamoy1]-2- | |
| methylpropyl](methyl)(aminocarbonyloxy)}methyl)-2-[(S)-2-[(S)-2-[3-(2-{N-2-[4-(4-amino-1-piperidyl)-3-(5,6-difluoro-1H-1,3- | |
| benzimidazol-2-yl)-5-(3-fluoro-5-tolyl)-2-pyridylamino]etbylcarbamoyl}ethoxy)propionylamino]-3- | |
| methylbutyrylamino]propionylamino]phenoxy]-3,4,5-trihydroxytetrahydro-2H-pyran-2-carboxylic acid | |
| 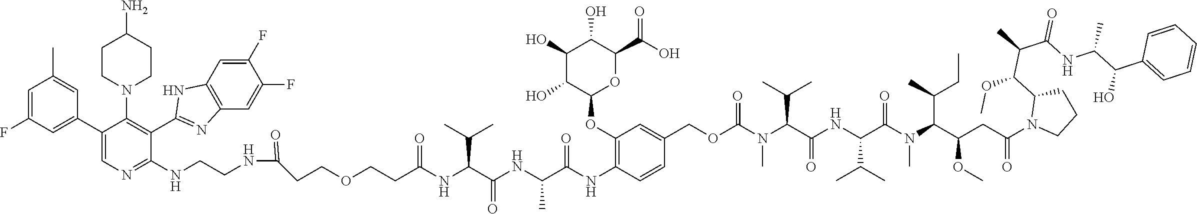 [SMILES: CC[C@H](C)[C@@H]([C@@H](CC(=O)N1CCC[C@H]1[C@H](OC)[C@@H](C)C(=O)N[C@H](C)[C@@H](O)c1ccccc1)OC)N(C)C(=O)[C@@H](NC(=O)[C@H](C(C)C)N(C)C(=O)OCc1ccc(NC(=O)[C@H](C)NC(=O)[C@@H](NC(=O)CCOCCC(=O)NCCNc2ncc(-c3cc(C)cc(F)c3)c(N3CCC(N)CC3)c2-c2nc3cc(F)c(F)cc3[nH]2)C(C)C)c(O[C@@H]2O[C@H](C(=O)O)[C@@H](O)[C@H](O)[C@H]2O)c1)C(C)C] | |
| (Compound 108) | |
| (2S,3S,4S,5R,6S)-6-[5-({[(S)-1-[N-(S)-1-{[(1S,2R)-4-[(S)-2-{(1R,2R)-2-[N-(1R,2S)-2-hydroxy-1-methyl-2-phenylethylcarbamoyl]-1- | 1850.9 |
| methoxypropyl}-1-pyrrolidiny1]-1-[(S)-1-methylpropyl]-2-methoxy-4-oxobutyl]-N-methylcarbamoyl}-2-methylpropylcarbamoyl]-2- | |
| methylpropyl](methyl)(aminocarbonyloxy)}methyl)-2-[(S)-2-[(S)-2-[3-(2-{N-2-[4-(4-amino-1-piperidyl)-3-(4,5-difluoro-1H-1,3- | |
| benzimidazol-2-yl)-5-(3-fluoro-5-tolyl)-2-pyridylamino]ethylcarbamoyl}ethoxy)propionylamino]-3- | |
| methylbutyrylamino]propionylamino]phenoxy]-3,4,5-trihydroxytetrahydro-2H-pyran-2-carboxylic acid | |
| 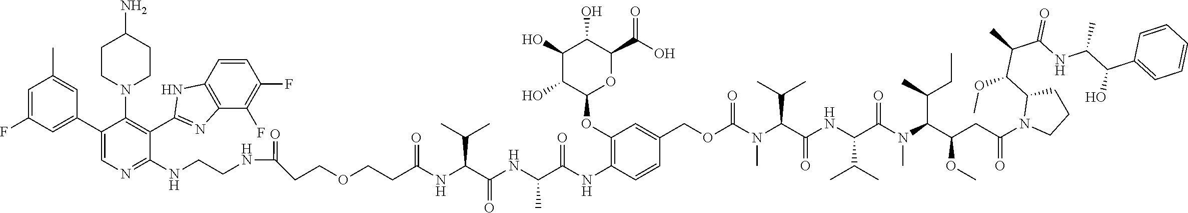 [SMILES: CC[C@H](C)[C@@H]([C@@H](CC(=O)N1CCC[C@H]1[C@H](OC)[C@@H](C)C(=O)N[C@H](C)[C@@H](O)c1ccccc1)OC)N(C)C(=O)[C@@H](NC(=O)[C@H](C(C)C)N(C)C(=O)OCc1ccc(NC(=O)[C@H](C)NC(=O)[C@@H](NC(=O)CCOCCC(=O)NCCNc2ncc(-c3cc(C)cc(F)c3)c(N3CCC(N)CC3)c2-c2nc3c(F)c(F)ccc3[nH]2)C(C)C)c(O[C@@H]2O[C@H](C(=O)O)[C@@H](O)[C@H](O)[C@H]2O)c1)C(C)C] | |
| (Compound 109) | |
| (2S,3S,4S,5R,6S)-6-[5-({[(S)-1-[N-(S)-1-{[(1S,2R)-4-[(S)-2-{(1R,2R)-2-[N-(1R,2S)-2-hydroxy-1-methyl-2-phenylethylcarbamoy1]-1- | 1937.0 |
| methoxypropyl}-1-pyrrolidinyl]-1-[(S)-1-methylpropyl]-2-methoxy-4-oxobutyl]-N-methylcarbamoyl}-2-methylpropylcarbamoyl]-2- | |
| methylpropyl](methyl)(aminocarbonyloxy)}methyl)-2-[(S)-2-[(S)-2-13-(2-{N-2-[4-(4-amino-1-piperidyl)-3-(4,5-difluoro-1H-1,3- | |
| benzimidazol-2-yl)-5-(3-fluoro-5-tolyl)-2-pyridylamino]ethylcarbamoyl}ethoxy)propionylamino]-3-methylbutyrylamino]-5- | |
| ureidovalerylamino]phenoxy]-3,4,5-trihydroxytetrahydro-2H-pyran-2-carboxylic acid | |
| 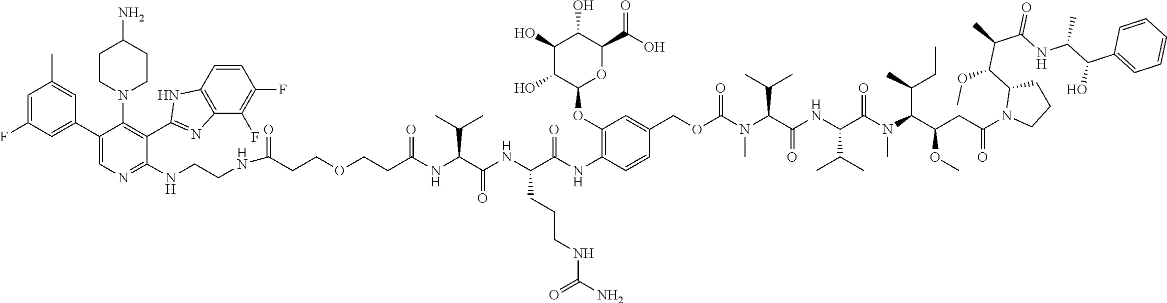 [SMILES: CC[C@H](C)[C@@H]([C@@H](CC(=O)N1CCC[C@H]1[C@H](OC)[C@@H](C)C(=O)N[C@H](C)[C@@H](O)c1ccccc1)OC)N(C)C(=O)[C@@H](NC(=O)[C@H](C(C)C)N(C)C(=O)OCc1ccc(NC(=O)[C@H](CCCNC(N)=O)NC(=O)[C@@H](NC(=O)CCOCCC(=O)NCCNc2ncc(-c3cc(C)cc(F)c3)c(N3CCC(N)CC3)c2-c2nc3c(F)c(F)ccc3[nH]2)C(C)C)c(O[C@@H]2O[C@H](C(=O)O)[C@@H](O)[C@H](O)[C@H]2O)c1)C(C)C] | |
| (Compound 110) | |
| (2S,3S,4S,5R,6S)-6-[5-({[(S)-1-[N-(S)-1-{[(1S,2R)-4-[(S)-2-{(1R,2R)-2-[N-(1R,2S)-2-hydroxy-1-methyl-2-phenylethylcarbamoyl]-1- | 1919.9 |
| methoxypropyl}-1-pyrrolidiny1]-1-[(S)-1-methylpropyl]-2-methoxy-4-oxobutyl]-N-methylcarbamoyl}-2-methylpropylcarbamoyl]-2- | |
| methylpropyl](methyl)(aminocarbonyloxy)}methyl)-2-[(S)-2-[(S)-2-[({N-methyl[2-(2-{N-2-[4-(4-amino-1-piperidyl)-3-(6-chloro-1H- | |
| 1,3-benzimidazol-2-yl)-5-(3-fluoro-5-tolyl)-2-pyridylamino]ethylcarbamoyl}ethoxy)ethyl\|carbonylamino}methyl)carbonylamino]-3- | |
| methylbutyrylamino]propionylamino]phenoxy]-3,4,5-trihydroxytetrahydro-2H-pyran-2-carboxylic acid | |
| 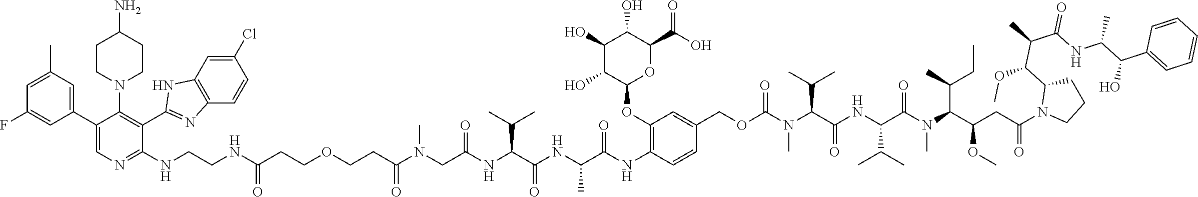 [SMILES: CC[C@H](C)[C@@H]([C@@H](CC(=O)N1CCC[C@H]1[C@H](OC)[C@@H](C)C(=O)N[C@H](C)[C@@H](O)c1ccccc1)OC)N(C)C(=O)[C@@H](NC(=O)[C@H](C(C)C)N(C)C(=O)OCc1ccc(NC(=O)[C@H](C)NC(=O)[C@@H](NC(=O)CN(C)C(=O)CCOCCC(=O)NCCNc2ncc(-c3cc(C)cc(F)c3)c(N3CCC(N)CC3)c2-c2nc3ccc(Cl)cc3[nH]2)C(C)C)c(O[C@@H]2O[C@H](C(=O)O)[C@@H](O)[C@H](O)[C@H]2O)c1)C(C)C] | |
| (Compound 111) | |
| (2S,3S,4S,5R,6S)-6-[5-({[(S)-1-[N-(S)-1-{[(1S,2R)-4-[(S)-2-{(1R,2R)-2-[N-(1R,2S)-2-hydroxy-1-methyl-2-phenylethylcarbamoyl]-1- | 1980.0 |
| methoxypropyl}-1-pyrrolidinyl]-1-[(S)-1-methylpropyl]-2-methoxy-4-oxobutyl]-N-methylcarbamoyl}-2-methylpropylcarbamoyl]-2- | |
| methylpropyl](methyl)(aminocarbonyloxy)}methy1)-2-[(S)-2-[(S)-2-(14-{N-2-[4-(4-amino-1-piperidyl)-3-(6-chloro-2-indolyl)-5-(3- | |
| fluoro-5-tolyl)-2-pyridylamino]ethylcarbamoyl}-3,6,9,12-tetraoxatetradecylcarbonylamino)-3- | |
| methylbutyrylamino]propionylamino]phenoxy]-3,4,5-trihydroxytetrahydro-2H-pyran-2-carboxylic acid | |
| 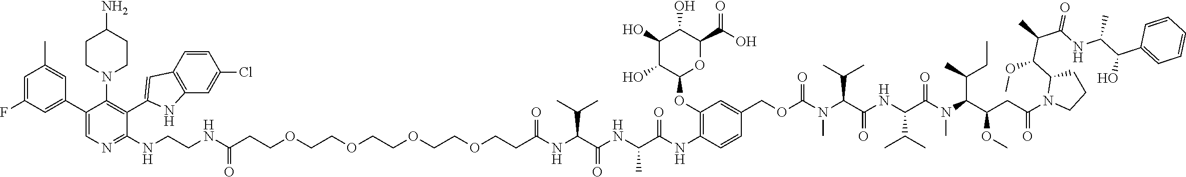 [SMILES: CC[C@H](C)[C@@H]([C@@H](CC(=O)N1CCC[C@H]1[C@H](OC)[C@@H](C)C(=O)N[C@H](C)[C@@H](O)c1ccccc1)OC)N(C)C(=O)[C@@H](NC(=O)[C@H](C(C)C)N(C)C(=O)OCc1ccc(NC(=O)[C@H](C)NC(=O)[C@@H](NC(=O)CCOCCOCCOCCOCCC(=O)NCCNc2ncc(-c3cc(C)cc(F)c3)c(N3CCC(N)CC3)c2-c2cc3ccc(Cl)cc3[nH]2)C(C)C)c(O[C@@H]2O[C@H](C(=O)O)[C@@H](O)[C@H](O)[C@H]2O)c1)C(C)C] | |
| (Compound 112) | |
| (2S,3S,4S,5R,6S)-6-[5-({[(S)-1-[N-(S)-1-{[(1S,2R)-4-[(S)-2-{(1R,2R)-2-[N-(1R,2S)-2-hydroxy-1-methyl-2-phenylethylcarbamoyl]-1- | 1891.0 |
| methoxypropyl}-1-pyrrolidinyl]-1-[(S)-1-methylpropyl]-2-methoxy-4-oxobutyl]-N-methylcarbamoyl}-2-methylpropylcarbamoyl]-2- | |
| methylpropyl](methyl)(aminocarbonyloxy)}methyl)-2-[(S)-2-[(S)-2-{3-[2-(N-2-{4-[(4aR,8aR)-octahydro-1H-4-oxa-1,6-diazanaphth- | |
| 6-yl]-3-(6-chloro-1H-1,3-benzimidazol-2-yl)-5-(3-fluoro-5-tolyl)-2-pyridylamino}ethylcarbamoyl)ethoxy]propionylamino}-3- | |
| methylbutyrylamino]propionylaminophenoxy]-3,4,5-trihydroxytetrahydro-2H-pyran-2-carboxylic acid | |
| 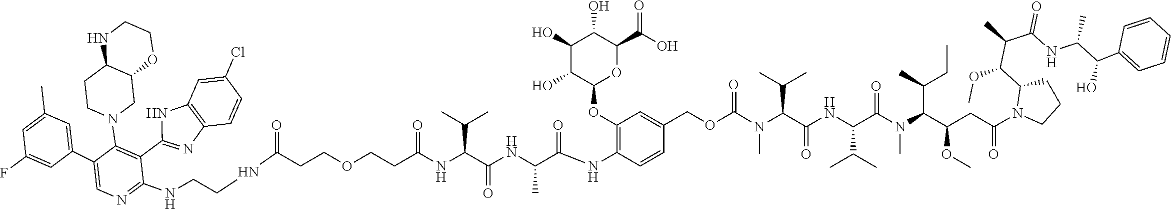 [SMILES: CC[C@H](C)[C@@H]([C@@H](CC(=O)N1CCC[C@H]1[C@H](OC)[C@@H](C)C(=O)N[C@H](C)[C@@H](O)c1ccccc1)OC)N(C)C(=O)[C@@H](NC(=O)[C@H](C(C)C)N(C)C(=O)OCc1ccc(NC(=O)[C@H](C)NC(=O)[C@@H](NC(=O)CCOCCC(=O)NCCNc2ncc(-c3cc(C)cc(F)c3)c(N3CC[C@H]4NCCO[C@@H]4C3)c2-c2nc3ccc(Cl)cc3[nH]2)C(C)C)c(O[C@@H]2O[C@H](C(=O)O)[C@@H](O)[C@H](O)[C@H]2O)c1)C(C)C] | |
| (Compound 113) | |
| (2S,3S,4S,5R,6S)-6-15-({[(S)-1-[N-(S)-1-{[(1S,2R)-4-[(S)-2-{(1R,2R)-2-[N-(1R,2S)-2-hydroxy-1-methyl-2-phenylethylcarbamoyl]-1- | 1892.9 |
| methoxypropyl}-1-pyrrolidinyl]-1-[(S)-1-methylpropyl]-2-methoxy-4-oxobutyl]-N-methylcarbamoyl}-2-methylpropylcarbamoyl]-2- | |
| methylpropyl](methyl)(aminocarbonyloxy)}methyl)-2-[(S)-2-[(S)-2-{3-[2-(N-2-{4-[(4aR,8aR)-octahydro-1H-4-oxa-1,6-diazanaphth- | |
| 6-yl]-3-(4,6-difluoro-1H-1,3-benzimidazol-2-yl)-5-(3-fluoro-5-tolyl)-2-pyridylamino}ethylcarbamoyl)ethoxy ]propionylamino}-3- | |
| methylbutyrylamino]propionylamino]phenoxy]-3,4,5-trihydroxytetrahydro-2H-pyran-2-carboxylic acid | |
| 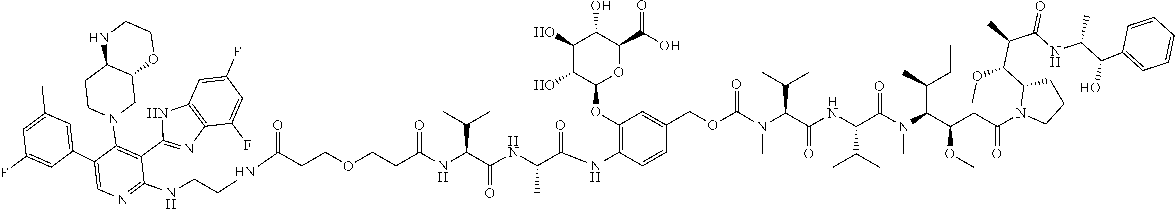 [SMILES: CC[C@H](C)[C@@H]([C@@H](CC(=O)N1CCC[C@H]1[C@H](OC)[C@@H](C)C(=O)N[C@H](C)[C@@H](O)c1ccccc1)OC)N(C)C(=O)[C@@H](NC(=O)[C@H](C(C)C)N(C)C(=O)OCc1ccc(NC(=O)[C@H](C)NC(=O)[C@@H](NC(=O)CCOCCC(=O)NCCNc2ncc(-c3cc(C)cc(F)c3)c(N3CC[C@H]4NCCO[C@@H]4C3)c2-c2nc3c(F)cc(F)cc3[nH]2)C(C)C)c(O[C@@H]2O[C@H](C(=O)O)[C@@H](O)[C@H](O)[C@H]2O)c1)C(C)C] | |
| (Compound 114) | |
| (2S,3S,4S,5R,6S)-6-[5-({[(S)-1-[N-(S)-1-{[(1S,2R)-4-[(S)-2-{(1R,2R)-2-[N-(1R,2S)-2-hydroxy-1-methyl-2-phenylethylcarbamoyl]-1- | 1847.9 |
| methoxypropyl}-1-pyrrolidiny1]-1-[(S)-1-methylpropyl]-2-methoxy-4-oxobutyl]-N-methylcarbamoyl}-2-methylpropylcarbamoyl]-2- | |
| methylpropyl](methyl)(aminocarbonyloxy)}methy1)-2-[(S)-2-[(S)-2-[3-(2-{N-2-[4-(4-amino-1-piperidyl)-3-(6-chloro-2-indoly1)-5-(3- | |
| fluoro-5-tolyl)-2-pyridylamino]ethylcarbamoyl}ethoxy)propionylamino]-3-methylbutyrylamino]propionylamino]phenoxy]-3,4,5- | |
| trihydroxytetrahydro-2H-pyran-2-carboxylic acid | |
| 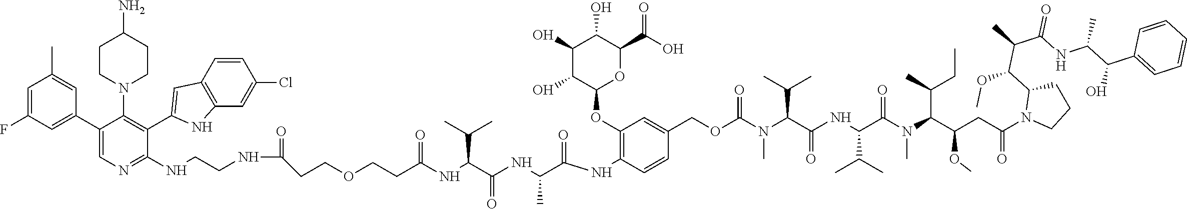 [SMILES: CC[C@H](C)[C@@H]([C@@H](CC(=O)N1CCC[C@H]1[C@H](OC)[C@@H](C)C(=O)N[C@H](C)[C@@H](O)c1ccccc1)OC)N(C)C(=O)[C@@H](NC(=O)[C@H](C(C)C)N(C)C(=O)OCc1ccc(NC(=O)[C@H](C)NC(=O)[C@@H](NC(=O)CCOCCC(=O)NCCNc2ncc(-c3cc(C)cc(F)c3)c(N3CCC(N)CC3)c2-c2cc3ccc(Cl)cc3[nH]2)C(C)C)c(O[C@@H]2O[C@H](C(=O)O)[C@@H](O)[C@H](O)[C@H]2O)c1)C(C)C] | |
| (Compound 115) | |
| (2S,3S,4S,5R,6S)-6-[5-({[(S)-1-[N-(S)-1-{[(1S,2R)-4-[(S)-2-{(1R,2R)-2-[N-(1R,2S)-2-hydroxy-1-methyl-2-phenylethylcarbamoyl]-1- | 1832.0 |
| methoxypropyl}-1-pyrrolidiny1]-1-[(S)-1-methylpropyl]-2-methoxy-4-oxobutyl]-N-methylcarbamoyl}-2-methylpropylcarbamoyl]-2- | |
| methylpropyl](methyl)(aminocarbonyloxy)}methyl)-2-[(S)-2-[(S)-2-[3-(2-{N-2-[4-(4-amino-1-piperidyl)-3-(6-fluoro-2-indolyl)-5-(3- | |
| fluoro-5-tolyl)-2-pyridylamino]ethylcarbamoyl}ethoxy)propionylamino]-3-methylbutyrylamino\|propionylamino]phenoxy]-3,4,5- | |
| trihydroxytetrahydro-2H-pyran-2-carboxylic acid | |
| 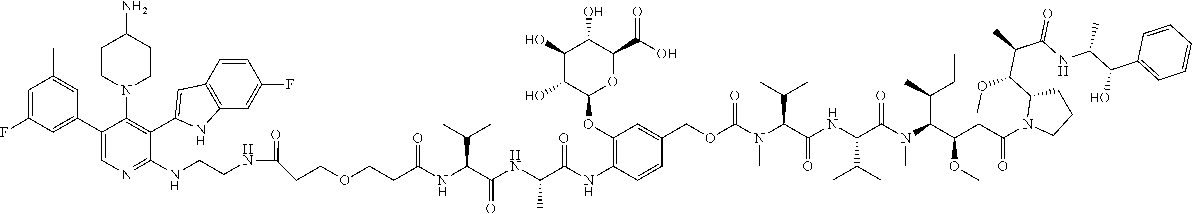 [SMILES: CC[C@H](C)[C@@H]([C@@H](CC(=O)N1CCC[C@H]1[C@H](OC)[C@@H](C)C(=O)N[C@H](C)[C@@H](O)c1ccccc1)OC)N(C)C(=O)[C@@H](NC(=O)[C@H](C(C)C)N(C)C(=O)OCc1ccc(NC(=O)[C@H](C)NC(=O)[C@@H](NC(=O)CCOCCC(=O)NCCNc2ncc(-c3cc(C)cc(F)c3)c(N3CCC(N)CC3)c2-c2cc3ccc(F)cc3[nH]2)C(C)C)c(O[C@@H]2O[C@H](C(=O)O)[C@@H](O)[C@H](O)[C@H]2O)c1)C(C)C] | |
| (Compound 116) | |
| (2S,3S,4S,5R,6S)-6-[5-({[(S)-1-[N-(S)-1-{[(1S,2R)-4-[(S)-2-{(1R,2R)-2-[N-(1R,2S)-2-hydroxy-1-methyl-2-phenylethylcarbamoyl]-1- | 1843.9 |
| methoxypropyl}-1-pyrrolidinyl]-1-[(S)-1-methylpropyl]-2-methoxy-4-oxobutyl]-N-methylcarbamoyl}-2-methylpropylcarbamoyl]-2- | |
| methylpropyl](methyl)(aminocarbonyloxy)}methyl)-2-[(S)-2-[(S)-2-[3-(2-{N-2-[4-(4-amino-1-piperidyl)-5-(3-fluoro-5-tolyl)-3-(6- | |
| methoxy-2-indolyl)-2-pyridylamino]ethylcarbamoyl}ethoxy)propionylamino]-3-methylbutyrylamino]propionylamino]phenoxy]-3,4,5- | |
| trihydroxytetrahydro-2H-pyran-2-carboxylic acid | |
| 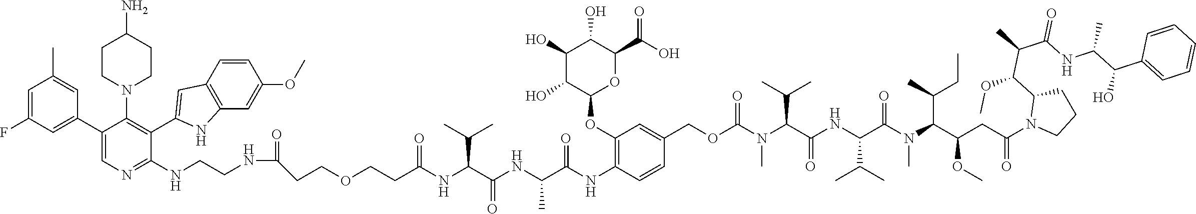 [SMILES: CC[C@H](C)[C@@H]([C@@H](CC(=O)N1CCC[C@H]1[C@H](OC)[C@@H](C)C(=O)N[C@H](C)[C@@H](O)c1ccccc1)OC)N(C)C(=O)[C@@H](NC(=O)[C@H](C(C)C)N(C)C(=O)OCc1ccc(NC(=O)[C@H](C)NC(=O)[C@@H](NC(=O)CCOCCC(=O)NCCNc2ncc(-c3cc(C)cc(F)c3)c(N3CCC(N)CC3)c2-c2cc3ccc(OC)cc3[nH]2)C(C)C)c(O[C@@H]2O[C@H](C(=O)O)[C@@H](O)[C@H](O)[C@H]2O)c1)C(C)C] | |
| (Compound 117) | |
| (2S,3S,4S,5R,6S)-6-[5-({[(S)-1-[N-(S)-1-{[(1S,2R)-4-[(S)-2-{(1R,2R)-2-[N-(1R,2S)-2-hydroxy-1-methyl-2-phenylethylcarbamoyl]-1- | 2006.0 |
| methoxypropyl}-1-pyrrolidinyl]-1-[(S)-1-methylpropyl]-2-methoxy-4-oxobutyl]-N-methylcarbamoyl}-2-methylpropylcarbamoyl]-2- | |
| methylpropyl](methyl)(aminocarbonyloxy)}methyl)-2-[(S)-2-[(S)-2-[({N-methyl[2-(2-{N-2-[4-(4-amino-1-piperidyl)-3-(6-chloro-1H- | |
| 1,3-benzimidazol-2-yl)-5-(3-fluoro-5-tolyl)-2-pyridylamino]ethylcarbamoyl}ethoxy)ethyl]carbonylamino}methyl)carbonylamino]-3- | |
| methylbutyrylamino]-5-ureidovalerylamino]phenoxy]-3,4,5-trihydroxytetrahydro-2H-pyran-2-carboxylic acid | |
| 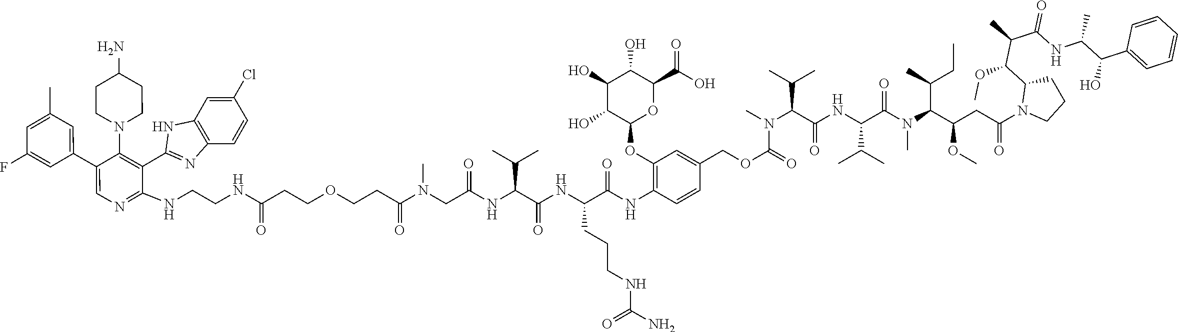 [SMILES: CC[C@H](C)[C@@H]([C@@H](CC(=O)N1CCC[C@H]1[C@H](OC)[C@@H](C)C(=O)N[C@H](C)[C@@H](O)c1ccccc1)OC)N(C)C(=O)[C@@H](NC(=O)[C@H](C(C)C)N(C)C(=O)OCc1ccc(NC(=O)[C@H](CCCNC(N)=O)NC(=O)[C@@H](NC(=O)CN(C)C(=O)CCOCCC(=O)NCCNc2ncc(-c3cc(C)cc(F)c3)c(N3CCC(N)CC3)c2-c2nc3ccc(Cl)cc3[nH]2)C(C)C)c(O[C@@H]2O[C@H](C(=O)O)[C@@H](O)[C@H](O)[C@H]2O)c1)C(C)C] | |
| (Compound 118) | |
| (2S,3S,4S,5R,6S)-6-[5-({[(S)-1-[N-(S)-1-{[(1S,2R)-4-[(S)-2-{(1R,2R)-2-[N-(1R,2S)-2-hydroxy-1-methyl-2-phenylethylcarbamoyl]-1- | 1839.0 |
| methoxypropyl}-1-pyrrolidinyl]-1-[(S)-1-methylpropyl]-2-methoxy-4-oxobutyl]-N-methylcarbamoyl}-2-methylpropylcarbamoyl]-2- | |
| methylpropyl](methyl)(aminocarbonyloxy)}methyl)-2-[(S)-2-[(S)-2-[3-(2-{N-2-[4-(4-amino-1-piperidyl)-3-(6-cyano-2-indoly1)-5-(3- | |
| fluoro-5-tolyl)-2-pyridylamino]ethylcarbamoyl}ethoxy)propionylamino]-3-methylbutyrylamino]propionylaminolphenoxy]-3,4,5- | |
| trihydroxytetrahydro-2H-pyran-2-carboxylic acid | |
| 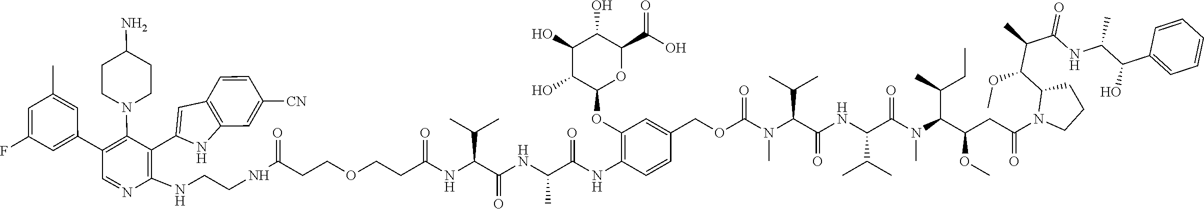 [SMILES: CC[C@H](C)[C@@H]([C@@H](CC(=O)N1CCC[C@H]1[C@H](OC)[C@@H](C)C(=O)N[C@H](C)[C@@H](O)c1ccccc1)OC)N(C)C(=O)[C@@H](NC(=O)[C@H](C(C)C)N(C)C(=O)OCc1ccc(NC(=O)[C@H](C)NC(=O)[C@@H](NC(=O)CCOCCC(=O)NCCNc2ncc(-c3cc(C)cc(F)c3)c(N3CCC(N)CC3)c2-c2cc3ccc(C#N)cc3[nH]2)C(C)C)c(O[C@@H]2O[C@H](C(=O)O)[C@@H](O)[C@H](O)[C@H]2O)c1)C(C)C] | |
| (Compound 119) | |
| (2S,3S,4S,5R,6S)-6-[5-({[(S)-1-[N-(S)-1-{[(1S,2R)-4-[(S)-2-{(1R,2R)-2-[N-(1R,2S)-2-hydroxy-1-methyl-2-phenylethylcarbamoyl]-1- | 1949.0 |
| methoxypropyl}-1-pyrrolidiny1]-1-[(S)-1-methylpropyl]-2-methoxy-4-oxobutyl]-N-methylcarbamoyl}-2-methylpropylcarbamoyl]-2- | |
| methylpropyl](methyl)(aminocarbonyloxy)}methyl)-2-[(S)-2-[(S)-2-[(R)-3-{N-2-[2-(N-2-{3-(6-chloro-1H-1,3-benzimidazol-2-yl)-5- | |
| (3-fluoro-5-tolyl)-4-[4-(methylamino)-1-piperidyl]-2-pyridylamino}ethylcarbamoyl)ethoxy]ethylcarbamoyl}-3- | |
| aminopropionylamino]-3-methylbutyrylamino]propionylamino]phenoxy]-3,4,5-trihydroxytetrahydro-2H-pyran-2-carboxylic acid | |
| 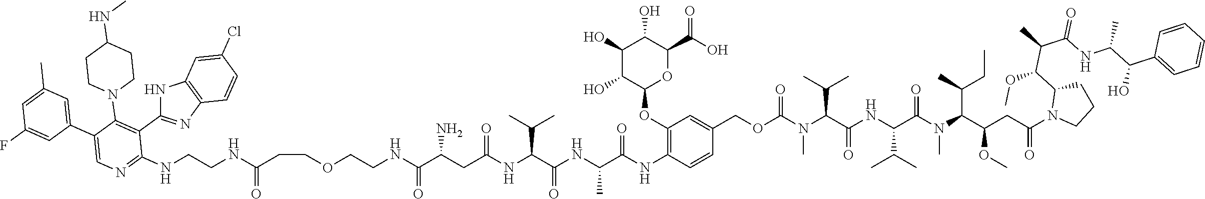 [SMILES: CC[C@H](C)[C@@H]([C@@H](CC(=O)N1CCC[C@H]1[C@H](OC)[C@@H](C)C(=O)N[C@H](C)[C@@H](O)c1ccccc1)OC)N(C)C(=O)[C@@H](NC(=O)[C@H](C(C)C)N(C)C(=O)OCc1ccc(NC(=O)[C@H](C)NC(=O)[C@@H](NC(=O)C[C@@H](N)C(=O)NCCOCCC(=O)NCCNc2ncc(-c3cc(C)cc(F)c3)c(N3CCC(NC)CC3)c2-c2nc3ccc(Cl)cc3[nH]2)C(C)C)c(O[C@@H]2O[C@H](C(=O)O)[C@@H](O)[C@H](O)[C@H]2O)c1)C(C)C] | |
| (Compound 120) | |
| (2S,3S,4S,5R,6S)-6-[5-({[(S)-1-[N-(S)-1-{[(1S,2R)-4-[(S)-2-{(1R,2R)-2-[N-(1R,2S)-2-hydroxy-1-methyl-2-phenylethylcarbamoyl]-1- | 2035.0 |
| methoxypropyl}-1-pyrrolidinyl]-1-[(S)-1-methylpropyl]-2-methoxy-4-oxobutyl]-N-methylcarbamoyl}-2-methylpropylcarbamoyl]-2- | |
| methylpropyl](methyl)(aminocarbonyloxy)}methyl)-2-[(S)-2-[(S)-2-[(R)-3-{N-2-[2-(N-2-{3-(6-chloro-1H-1,3-benzimidazol-2-yl)-5- | |
| (3-fluoro-5-tolyl)-4-[4-(methylamino)-1-piperidyl]-2-pyridylamino}ethylcarbamoyl)ethoxy]ethylcarbamoyl}-3- | |
| aminopropionylamino]-3-methylbutyrylamino]-5-ureidovalerylamino]phenoxy]-3,4,5-trihydroxytetrahydro-2H-pyran-2-carboxylic | |
| acid | |
| 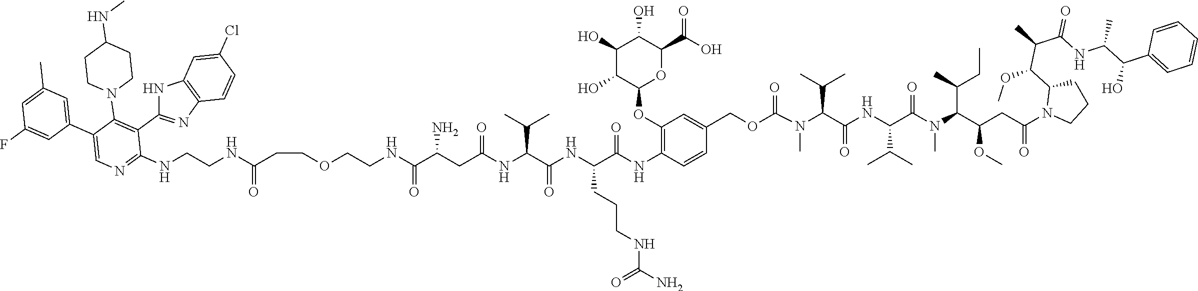 [SMILES: CC[C@H](C)[C@@H]([C@@H](CC(=O)N1CCC[C@H]1[C@H](OC)[C@@H](C)C(=O)N[C@H](C)[C@@H](O)c1ccccc1)OC)N(C)C(=O)[C@@H](NC(=O)[C@H](C(C)C)N(C)C(=O)OCc1ccc(NC(=O)[C@H](CCCNC(N)=O)NC(=O)[C@@H](NC(=O)C[C@@H](N)C(=O)NCCOCCC(=O)NCCNc2ncc(-c3cc(C)cc(F)c3)c(N3CCC(NC)CC3)c2-c2nc3ccc(Cl)cc3[nH]2)C(C)C)c(O[C@@H]2O[C@H](C(=O)O)[C@@H](O)[C@H](O)[C@H]2O)c1)C(C)C] | |
| (Compound 121) | |
| (2S,3S,4S,5R,6S)-6-[5-({[(S)-1-[N-(S)-1-{[(1S,2R)-4-[(S)-2-{(1R,2R)-2-[N-(1R,2S)-2-hydroxy-1-methyl-2-phenylethylcarbamoyl]-1- | 2167.1 |
| methoxypropyl}-1-pyrrolidinyl]-1-[(S)-1-methylpropyl]-2-methoxy-4-oxobutyl]-N-methylcarbamoyl}-2-methylpropylcarbamoyl]-2- | |
| methylpropyl](methyl)(aminocarbonyloxy)}methyl)-2-[(S)-2-[(S)-2-[(R)-3-[N-14-(N-2-{3-(6-chloro-1H-1,3-benzimidazol-2-yl)-5-(3- | |
| fluoro-5-tolyl)-4-[4-(methylamino)-1-piperidyl]-2-pyridylamino}ethylcarbamoyl)-3,6,9,12-tetraoxatetradecylcarbamoyl]-3- | |
| aminopropionylamino]-3-methylbutyrylamino]-5-ureidovalerylamino]phenoxy]-3,4,5-trihydroxytetrahydro-2H-pyran-2-carboxylic | |
| acid | |
| 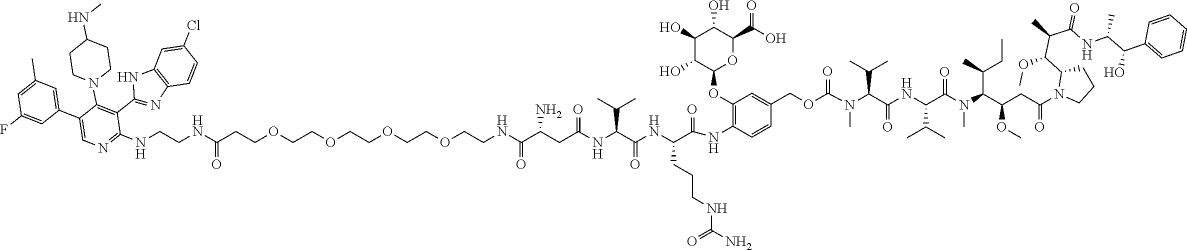 [SMILES: CC[C@H](C)[C@@H]([C@@H](CC(=O)N1CCC[C@H]1[C@H](OC)[C@@H](C)C(=O)N[C@H](C)[C@@H](O)c1ccccc1)OC)N(C)C(=O)[C@@H](NC(=O)[C@H](C(C)C)N(C)C(=O)OCc1ccc(NC(=O)[C@H](CCCNC(N)=O)NC(=O)[C@@H](NC(=O)C[C@@H](N)C(=O)NCCOCCOCCOCCOCCC(=O)NCCNc2ncc(-c3cc(C)cc(F)c3)c(N3CCC(NC)CC3)c2-c2nc3ccc(Cl)cc3[nH]2)C(C)C)c(O[C@@H]2O[C@H](C(=O)O)[C@@H](O)[C@H](O)[C@H]2O)c1)C(C)C] | |
| (Compound 122) | |
| (2S,3S,4S,5R,6S)-6-[5-({[(S)-1-[N-(S)-1-{[(1S,2R)-4-[(S)-2-{(1R,2R)-2-[N-(1R,2S)-2-hydroxy-1-methyl-2-phenylethylcarbamoyl]-1- | 2035.0 |
| methoxypropyl}-1-pyrrolidiny1]-1-[(S)-1-methylpropyl]-2-methoxy-4-oxobutyl]-N-methylcarbamoyl}-2-methylpropylcarbamoyl]-2- | |
| methylpropyl](methyl)(aminocarbonyloxy)}methyl)-2-[(S)-2-[(S)-2-[(R)-3-[N-2-(2-{N-2-[4-(4-amino-1-piperidyl)-3-(5-fluoro-7- | |
| methoxy-1H-1,3-benzimidazol-2-yl)-5-(3-fluoro-5-tolyl)-2-pyridylamino]ethylcarbamoyl}ethoxy)ethylcarbamoy1]-3- | |
| aminopropionylamino]-3-methylbutyrylamino]-5-ureidovalerylamino]phenoxy]-3,4,5-trihydroxytetrahydro-2H-pyran-2-carboxylic | |
| acid | |
| 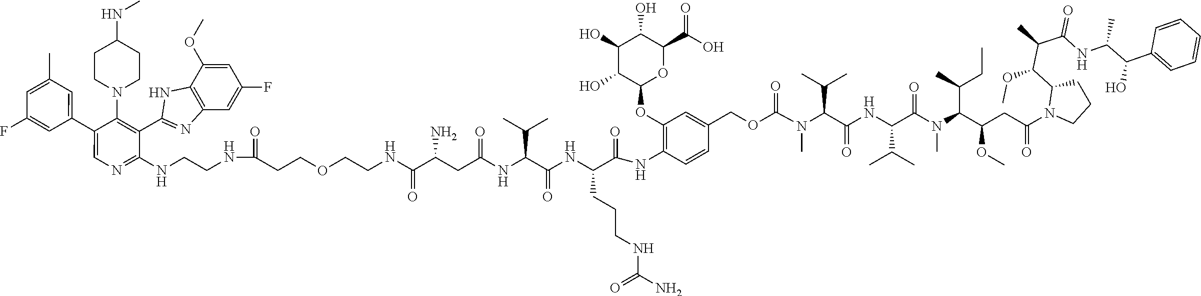 [SMILES: CC[C@H](C)[C@@H]([C@@H](CC(=O)N1CCC[C@H]1[C@H](OC)[C@@H](C)C(=O)N[C@H](C)[C@@H](O)c1ccccc1)OC)N(C)C(=O)[C@@H](NC(=O)[C@H](C(C)C)N(C)C(=O)OCc1ccc(NC(=O)[C@H](CCCNC(N)=O)NC(=O)[C@@H](NC(=O)C[C@@H](N)C(=O)NCCOCCC(=O)NCCNc2ncc(-c3cc(C)cc(F)c3)c(N3CCC(NC)CC3)c2-c2nc3cc(F)cc(OC)c3[nH]2)C(C)C)c(O[C@@H]2O[C@H](C(=O)O)[C@@H](O)[C@H](O)[C@H]2O)c1)C(C)C] | |
| (Compound 123) | |
| (2S,3S,4S,5R,6S)-3,4,5-trihydroxy-6-{2-[(S)-2-[(S)-2-[(S)-3-hydroxy-2-[3-(2-{N-2-[4-(4-amino-1-piperidyl)-3-(6-chloro-1H-1,3- | 1935.9 |
| benzimidazol-2-yl)-5-(3-fluoro-5-tolyl)-2-pyridylamino]ethylcarbamoyl}ethoxy)propionylamino]propionylamino]-3- | |
| methylbutyrylamino]propionylamino]-5-({methyl[(S)-2-methyl-1-[N-(S)-1-{[(1S,2R)-2-methoxy-4-[(S)-2-[(1R,2R)-1-methoxy-2-[N- | |
| (1R,2S)-2-hydroxy-1-methyl-2-phenylethylcarbamoyl\|propyl]-1-pyrrolidinyl]-1-[(S)-1-methylpropyl]-4-oxobutyl]-N- | |
| methylcarbamoyl}-2-methylpropylcarbamoyl]propyl](aminocarbonyloxy)}methyl)phenoxy}tetrahydro-2H-pyran-2-carboxylic acid | |
| 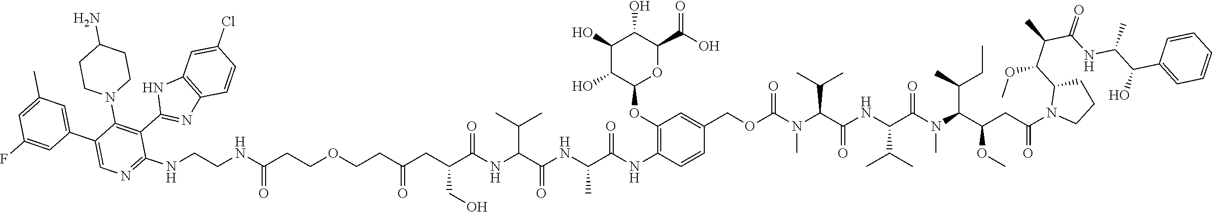 [SMILES: CC[C@H](C)[C@@H]([C@@H](CC(=O)N1CCC[C@H]1[C@H](OC)[C@@H](C)C(=O)N[C@H](C)[C@@H](O)c1ccccc1)OC)N(C)C(=O)[C@@H](NC(=O)[C@H](C(C)C)N(C)C(=O)OCc1ccc(NC(=O)[C@H](C)NC(=O)C(NC(=O)[C@H](CO)CC(=O)CCOCCC(=O)NCCNc2ncc(-c3cc(C)cc(F)c3)c(N3CCC(N)CC3)c2-c2nc3ccc(Cl)cc3[nH]2)C(C)C)c(O[C@@H]2O[C@H](C(=O)O)[C@@H](O)[C@H](O)[C@H]2O)c1)C(C)C] | |
| (Compound 124) | |
| (2S,3S,4S,5R,6S)-6-[5-({[(S)-1-[N-(S)-1-{[(1S,2R)-4-[(S)-2-{(1R,2R)-2-[N-(1R,2S)-2-hydroxy-1-methyl-2-phenylethylcarbamoyl]-1- | 1862.9 |
| methoxypropyl}-1-pyrrolidinyl]-1-[(S)-1-methylpropyl]-2-methoxy-4-oxobutyl]-N-methylcarbamoyl}-2-methylpropylcarbamoyl]-2- | |
| methylpropyl](methyl)(aminocarbonyloxy)}methyl)-2-[(S)-2-[(S)-2-{3-[2-(N-2-{[4-(4-amino-1-piperidyl)-3-(6-chloro-1H-1,3- | |
| benzimidazol-2-yl)-5-(3-fluoro-5-tolyl)-2-pyridyl]-N-methylamino}ethylcarbamoyl)ethoxy]propionylamino}-3- | |
| methylbutyrylamino]propionylamino]phenoxy]-3,4,5-trihydroxytetrahydro-2H-pyran-2-carboxylic acid | |
| 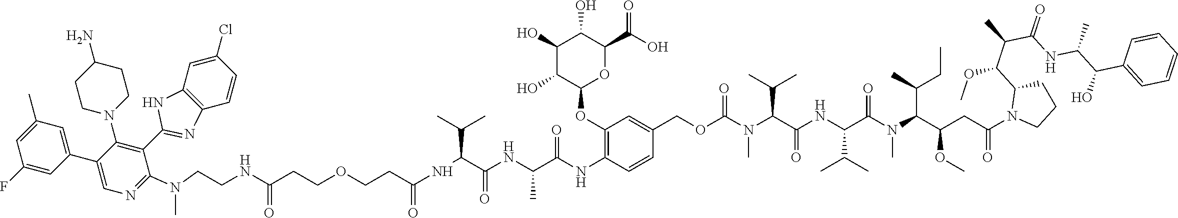 [SMILES: CC[C@H](C)[C@@H]([C@@H](CC(=O)N1CCC[C@H]1[C@H](OC)[C@@H](C)C(=O)N[C@H](C)[C@@H](O)c1ccccc1)OC)N(C)C(=O)[C@@H](NC(=O)[C@H](C(C)C)N(C)C(=O)OCc1ccc(NC(=O)[C@H](C)NC(=O)[C@@H](NC(=O)CCOCCC(=O)NCCN(C)c2ncc(-c3cc(C)cc(F)c3)c(N3CCC(N)CC3)c2-c2nc3ccc(Cl)cc3[nH]2)C(C)C)c(O[C@@H]2O[C@H](C(=O)O)[C@@H](O)[C@H](O)[C@H]2O)c1)C(C)C] | |
| (Compound 125) | |
| (2S,3S,4S,5R,6S)-6-[5-({[(S)-1-[N-(S)-1-{[(1S,2R)-4-[(S)-2-{(1R,2R)-2-[N-(1R,2S)-2-hydroxy-1-methyl-2-phenylethylcarbamoyl]-1- | 1849.9 |
| methoxypropyl}-1-pyrrolidinyl]-1-[(S)-1-methylpropyl]-2-methoxy-4-oxobutyl]-N-methylcarbamoyl}-2-methylpropylcarbamoyl]-2- | |
| methylpropyl](methyl)(aminocarbonyloxy)}methyl)-2-[(S)-2-[(S)-2-13-(2-(N-2-[4-(4-amino-1-piperidyl)-3-(6-chloro-1H-1,3,4- | |
| triazainden-2-yl)-5-(3-fluoro-5-tolyl)-2-pyridylamino]ethylcarbamoyl}ethoxy)propionylamino]-3- | |
| methylbutyrylamino]propionylamino]phenoxy]-3,4,5-trihydroxytetrahydro-2H-pyran-2-carboxylic acid | |
| 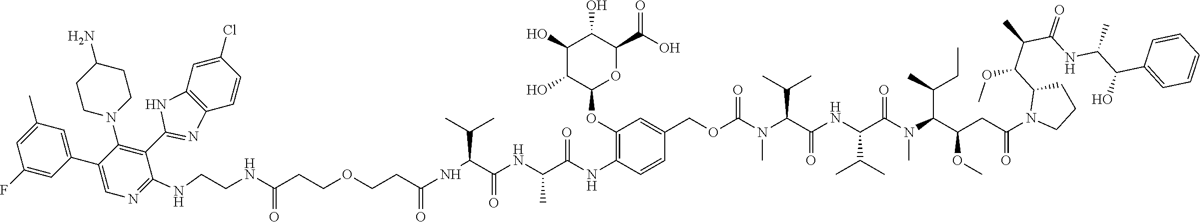 [SMILES: CC[C@H](C)[C@@H]([C@@H](CC(=O)N1CCC[C@H]1[C@H](OC)[C@@H](C)C(=O)N[C@H](C)[C@@H](O)c1ccccc1)OC)N(C)C(=O)[C@@H](NC(=O)[C@H](C(C)C)N(C)C(=O)OCc1ccc(NC(=O)[C@H](C)NC(=O)[C@@H](NC(=O)CCOCCC(=O)NCCNc2ncc(-c3cc(C)cc(F)c3)c(N3CCC(N)CC3)c2-c2nc3ccc(Cl)cc3[nH]2)C(C)C)c(O[C@@H]2O[C@H](C(=O)O)[C@@H](O)[C@H](O)[C@H]2O)c1)C(C)C] | |
| (Compound 126) | |
| (2S,3S,4S,5R,6S)-6-[5-({[(S)-1-[N-(S)-1-{[(1S,2R)-4-[(S)-2-{(1R,2R)-2-[N-(1R,2S)-2-hydroxy-1-methyl-2-phenylethylcarbamoyl]-1- | 1815.9 |
| methoxypropyl}-1-pyrrolidinyl]-1-[(S)-1-methylpropyl]-2-methoxy-4-oxobutyl]-N-methylcarbamoyl}-2-methylpropylcarbamoyl]-2- | |
| methylpropyl](methyl)(aminocarbonyloxy)}methy1)-2-[(S)-2-[(S)-2-[3-(2-{N-2-[4-(4-amino-1-piperidyl)-5-(3-fluoro-5-toly1)-3-(1H- | |
| 1,3,5-triazainden-2-yl)-2-pyridylamino]ethylcarbamoyl)ethoxy)propionylamino]-3-methylbutyrylamino]propionylamino]phenoxy]- | |
| 3,4,5-trihydroxytetrahydro-2H-pyran-2-carboxylic acid | |
| 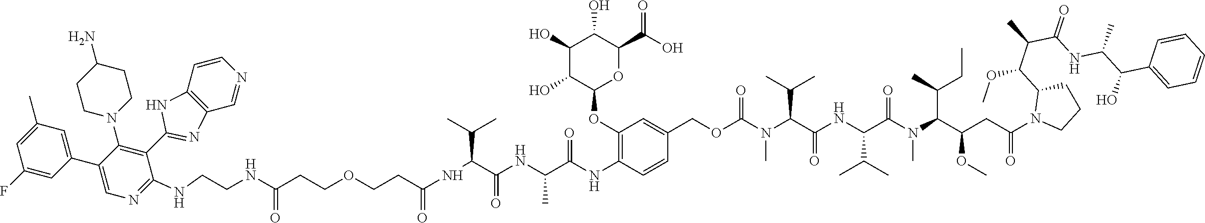 [SMILES: CC[C@H](C)[C@@H]([C@@H](CC(=O)N1CCC[C@H]1[C@H](OC)[C@@H](C)C(=O)N[C@H](C)[C@@H](O)c1ccccc1)OC)N(C)C(=O)[C@@H](NC(=O)[C@H](C(C)C)N(C)C(=O)OCc1ccc(NC(=O)[C@H](C)NC(=O)[C@@H](NC(=O)CCOCCC(=O)NCCNc2ncc(-c3cc(C)cc(F)c3)c(N3CCC(N)CC3)c2-c2nc3cnccc3[nH]2)C(C)C)c(O[C@@H]2O[C@H](C(=O)O)[C@@H](O)[C@H](O)[C@H]2O)c1)C(C)C] | |
| (Compound 127) | |
| (2S,3S,4S,5R,6S)-6-[5-({[(S)-1-[N-(S)-1-{[(1S,2R)-4-[(S)-2-{(1R,2R)-2-[N-(1R,2S)-2-hydroxy-1-methyl-2-phenylethylcarbamoyl]-1- | 1832.1 |
| methoxypropyl}-1-pyrrolidiny1]-1-[(S)-1-methylpropyl]-2-methoxy-4-oxobutyl]-N-methylcarbamoyl}-2-methylpropylcarbamoyl]-2- | |
| methylpropyl](methyl)(aminocarbonyloxy)}methyl)-2-[(S)-2-[(S)-2-[3-(2-{N-2-[4-(4-amino-1-piperidyl)-5-(6-chloro-1H-1,3- | |
| benzimidazol-2-yl)-2′-methyl-3,4′-bipyridyl-6-ylamino]ethylcarbamoyl}ethoxy)propionylamino]-3- | |
| methylbutyrylamino]propionylamino]phenoxy]-3,4,5-trihydroxytetrahydro-2H-pyran-2-carboxylic acid | |
| 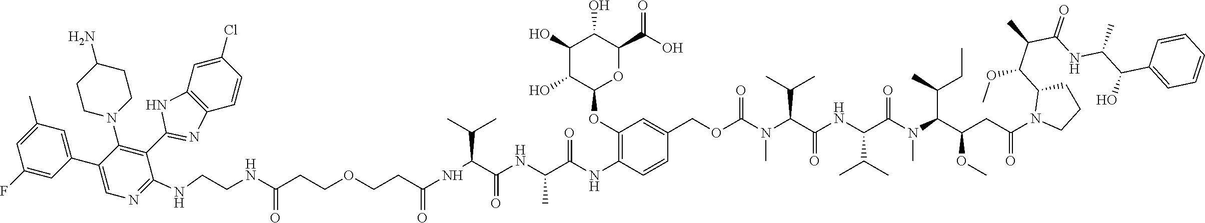 [SMILES: CC[C@H](C)[C@@H]([C@@H](CC(=O)N1CCC[C@H]1[C@H](OC)[C@@H](C)C(=O)N[C@H](C)[C@@H](O)c1ccccc1)OC)N(C)C(=O)[C@@H](NC(=O)[C@H](C(C)C)N(C)C(=O)OCc1ccc(NC(=O)[C@H](C)NC(=O)[C@@H](NC(=O)CCOCCC(=O)NCCNc2ncc(-c3cc(C)cc(F)c3)c(N3CCC(N)CC3)c2-c2nc3ccc(Cl)cc3[nH]2)C(C)C)c(O[C@@H]2O[C@H](C(=O)O)[C@@H](O)[C@H](O)[C@H]2O)c1)C(C)C] | |
| (Compound 128) | |
| (2S,3S,4S,5R,6S)-6-[5-({[(S)-1-[N-(S)-1-{[(1S,2R)-4-[(S)-2-{(1R,2R)-2-[N-(1R,2S)-2-hydroxy-1-methyl-2-phenylethylcarbamoyl]-1- | 1831.9 |
| methoxypropyl}-1-pyrrolidinyl]-1-[(S)-1-methylpropyl]-2-methoxy-4-oxobutyl]-N-methylcarbamoyl}-2-methylpropylcarbamoyl]-2- | |
| methylpropyl](methyl)(aminocarbonyloxy)}methyl)-2-[(S)-2-[(S)-2-[3-(2-{N-2-[4-(4-amino-1-piperidyl)-5-(6-chloro-1H-1,3- | |
| benzimidazol-2-yl)-5′-methyl-3,3′-bipyridyl-6-ylamino]ethylcarbamoyl}ethoxy)propionylamino]-3- | |
| methylbutyrylamino]propionylamino]phenoxy]-3,4,5-trihydroxytetrahydro-2H-pyran-2-carboxylic acid | |
| 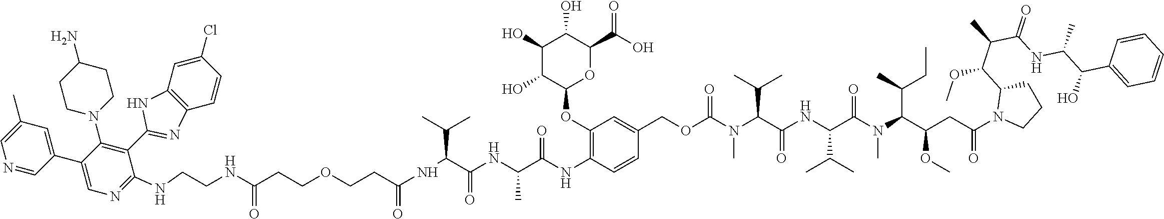 [SMILES: CC[C@H](C)[C@@H]([C@@H](CC(=O)N1CCC[C@H]1[C@H](OC)[C@@H](C)C(=O)N[C@H](C)[C@@H](O)c1ccccc1)OC)N(C)C(=O)[C@@H](NC(=O)[C@H](C(C)C)N(C)C(=O)OCc1ccc(NC(=O)[C@H](C)NC(=O)[C@@H](NC(=O)CCOCCC(=O)NCCNc2ncc(-c3cncc(C)c3)c(N3CCC(N)CC3)c2-c2nc3ccc(Cl)cc3[nH]2)C(C)C)c(O[C@@H]2O[C@H](C(=O)O)[C@@H](O)[C@H](O)[C@H]2O)c1)C(C)C] | |
| (Compound 129) | |
| (2S,3S,4S,5R,6S)-6-[5-({[(S)-1-[N-(S)-1-{[(1S,2R)-4-[(S)-2-{(1R,2R)-2-[N-(1R,2S)-2-hydroxy-1-methyl-2-phenylethylcarbamoyl]-1- | 2118.0 |
| methoxypropyl}-1-pyrrolidinyl]-1-[(S)-1-methylpropyl]-2-methoxy-4-oxobutyl]-N-methylcarbamoyl}-2-methylpropylcarbamoyl]-2- | |
| methylpropyl](methyl)(aminocarbonyloxy)}methyl)-2-[(S)-2-[(S)-2-(4-{[({[({N-2-[4-(4-amino-1-piperidyl)-3-(6-chloro-1H-1,3- | |
| benzimidazol-2-yl)-5-(3-fluoro-5-tolyl)-2-pyridylamino]ethylcarbamoyl}methyl)-N-methylcarbamoyl]methyl}-N- | |
| methylcarbamoyl)methyl]-N-methylcarbamoyl}butyrylamino)-3-methylbutyrylamino]-5-ureidovalerylamino\|phenoxy]-3,4,5- | |
| trihydroxytetrahydro-2H-pyran-2-carboxylic acid | |
| 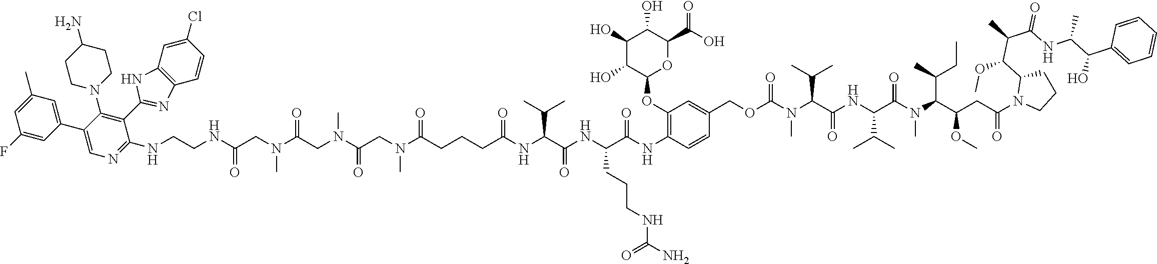 [SMILES: CC[C@H](C)[C@@H]([C@@H](CC(=O)N1CCC[C@H]1[C@H](OC)[C@@H](C)C(=O)N[C@H](C)[C@@H](O)c1ccccc1)OC)N(C)C(=O)[C@@H](NC(=O)[C@H](C(C)C)N(C)C(=O)OCc1ccc(NC(=O)[C@H](CCCNC(N)=O)NC(=O)[C@@H](NC(=O)CCCC(=O)N(C)CC(=O)N(C)CC(=O)N(C)CC(=O)NCCNc2ncc(-c3cc(C)cc(F)c3)c(N3CCC(N)CC3)c2-c2nc3ccc(Cl)cc3[nH]2)C(C)C)c(O[C@@H]2O[C@H](C(=O)O)[C@@H](O)[C@H](O)[C@H]2O)c1)C(C)C] | |
| (Compound 130) | |
| (2S,3S,4S,5R,6S)-6-[5-({[(S)-1-[N-(S)-1-{[(1S,2R)-4-[(S)-2-{(1R,2R)-2-[N-(1R,2S)-2-hydroxy-1-methyl-2-phenylethylcarbamoyl]-1- | 2001.9 |
| methoxypropyl}-1-pyrrolidinyl]-1-[(S)-1-methylpropyl]-2-methoxy-4-oxobutyl]-N-methylcarbamoyl}-2-methylpropylcarbamoyl]-2- | |
| methylpropyl](methyl)(aminocarbonyloxy)}methyl)-2-[(S)-2-[(S)-2-{1-[2-(2-{N-2-[4-(4-amino-1-piperidyl)-3-(6-chloro-1H-1,3- | |
| benzimidazol-2-yl)-5-(3-fluoro-5-tolyl)-2-pyridylamino]ethylcarbamoyl}ethoxy)ethyl]-1H-1,2,3-triazol-4-ylcarbonylamino}-3- | |
| methylbutyrylamino]-5-ureidovalerylamino]phenoxy]-3,4,5-trihydroxytetrahydro-2H-pyran-2-carboxylic acid | |
| 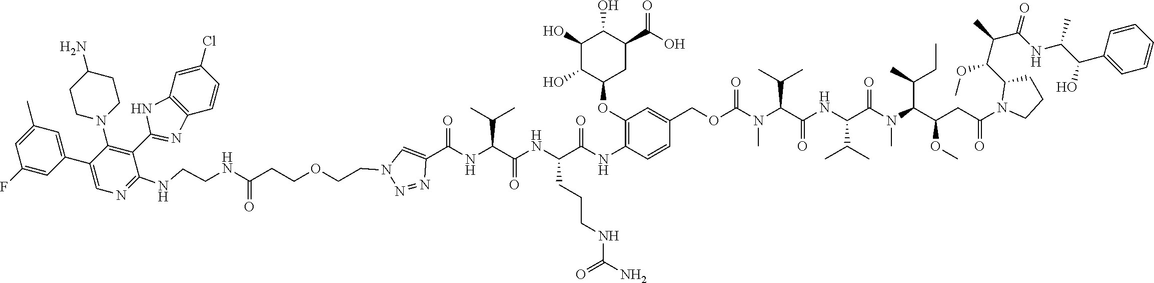 [SMILES: CC[C@H](C)[C@@H]([C@@H](CC(=O)N1CCC[C@H]1[C@H](OC)[C@@H](C)C(=O)N[C@H](C)[C@@H](O)c1ccccc1)OC)N(C)C(=O)[C@@H](NC(=O)[C@H](C(C)C)N(C)C(=O)OCc1ccc(NC(=O)[C@H](CCCNC(N)=O)NC(=O)[C@@H](NC(=O)c2cn(CCOCCC(=O)NCCNc3ncc(-c4cc(C)cc(F)c4)c(N4CCC(N)CC4)c3-c3nc4ccc(Cl)cc4[nH]3)nn2)C(C)C)c(O[C@@H]2C[C@H](C(=O)O)[C@@H](O)[C@H](O)[C@H]2O)c1)C(C)C] | |
| (Compound 131) | |
| (2S,3S,4S,5R,6S)-6-[5-({[(S)-1-[N-(S)-1-{[(1S,2R)-4-[(S)-2-{(1R,2R)-2-[N-(1R,2S)-2-hydroxy-1-methyl-2-phenylethylcarbamoyl]-1- | 2056.0 |
| methoxypropyl}-1-pyrrolidiny1]-1-[(S)-1-methylpropyl]-2-methoxy-4-oxobutyl\|-N-methylcarbamoyl}-2-methylpropylcarbamoyl]-2- | |
| methylpropyl](methyl)(aminocarbonyloxy)}methyl)-2-[(S)-2-[(S)-2-[(S)-2-[3-(2-{N~2-[4-(4-amino-1-piperidy])-3-(6-chloro-1H-1,3- | |
| benzimidazol-2-yl)-5-(3-fluoro-5-tolyl)-2-pyridylamino]ethylcarbamoyl}ethoxy)propionylamino\|propylsulfonylamino]-3- | |
| methylbutyrylamino]-5-ureidovalerylamino]phenoxy]-3,4,5-trihydroxytetrahydro-2H-pyran-2-carboxylic acid | |
| 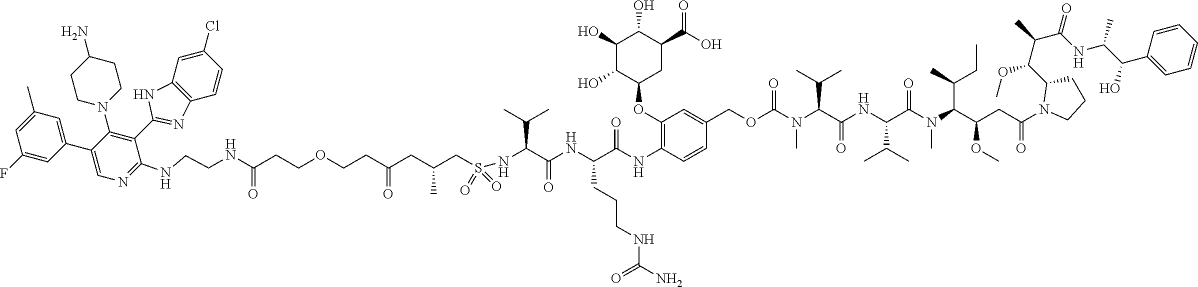 [SMILES: CC[C@H](C)[C@@H]([C@@H](CC(=O)N1CCC[C@H]1[C@H](OC)[C@@H](C)C(=O)N[C@H](C)[C@@H](O)c1ccccc1)OC)N(C)C(=O)[C@@H](NC(=O)[C@H](C(C)C)N(C)C(=O)OCc1ccc(NC(=O)[C@H](CCCNC(N)=O)NC(=O)[C@@H](NS(=O)(=O)C[C@H](C)CC(=O)CCOCCC(=O)NCCNc2ncc(-c3cc(C)cc(F)c3)c(N3CCC(N)CC3)c2-c2nc3ccc(Cl)cc3[nH]2)C(C)C)c(O[C@@H]2C[C@H](C(=O)O)[C@@H](O)[C@H](O)[C@H]2O)c1)C(C)C] | |
| (Compound 132) | |
| (2S,3S,4S,5R,6S)-6-[5-({[(S)-1-[N-(S)-1-{[(1S,2R)-4-[(S)-2-{(1R,2R)-2-[N-(1R,2S)-2-hydroxy-1-methyl-2-phenylethylcarbamoyl]-1- | 2006.0 |
| methoxypropyl}-1-pyrrolidinyl]-1-[(S)-1-methylpropyl]-2-methoxy-4-oxobutyl]-N-methylcarbamoyl}-2-methylpropylcarbamoyl]-2- | |
| methylpropyl](methyl)(aminocarbonyloxy)}methyl)-2-[(S)-2-[(S)-2-(3-{2-[({N-2-[4-(4-amino-1-piperidyl)-3-(6-chloro-1H-1,3- | |
| benzimidazol-2-yl)-5-(3-fluoro-5-tolyl)-2-pyridylamino]ethylcarbamoyl}methyl)-N-methylcarbamoyl]ethoxy}propionylamino)-3- | |
| methylbutyrylamino]-5-ureidovalerylamino]phenoxy]-3,4,5-trihydroxytetrahydro-2H-pyran-2-carboxylic acid | |
| 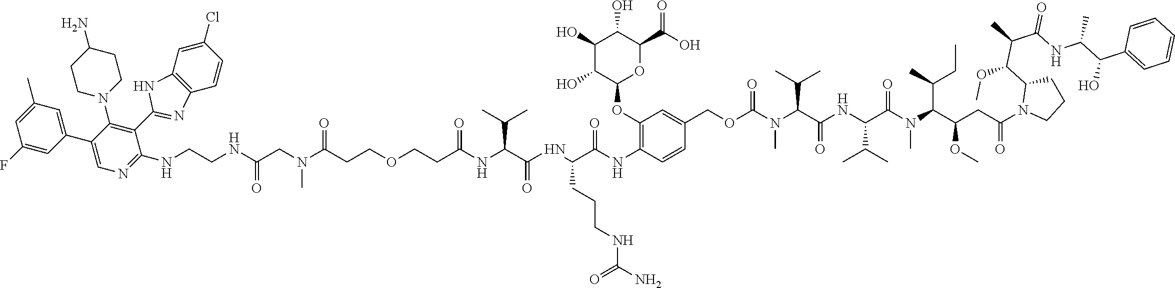 [SMILES: CC[C@H](C)[C@@H]([C@@H](CC(=O)N1CCC[C@H]1[C@H](OC)[C@@H](C)C(=O)N[C@H](C)[C@@H](O)c1ccccc1)OC)N(C)C(=O)[C@@H](NC(=O)[C@H](C(C)C)N(C)C(=O)OCc1ccc(NC(=O)[C@H](CCCNC(N)=O)NC(=O)[C@@H](NC(=O)CCOCCC(=O)N(C)CC(=O)NCCNc2ncc(-c3cc(C)cc(F)c3)c(N3CCC(N)CC3)c2-c2nc3ccc(Cl)cc3[nH]2)C(C)C)c(O[C@@H]2O[C@H](C(=O)O)[C@@H](O)[C@H](O)[C@H]2O)c1)C(C)C] | |
| (Compound 133) | |
| (2S,3S,4S,5R,6S)-6-[5-({[(S)-1-[N-(S)-1-{[(1S,2R)-4-[(S)-2-{(1R,2R)-2-[N-(1R,2S)-2-hydroxy-1-methyl-2-phenylethylcarbamoyl]-1- | 1919.9 |
| methoxypropyl}-1-pyrrolidinyl]-1-[(S)-1-methylpropyl]-2-methoxy-4-oxobutyl\|-N-methylcarbamoyl}-2-methylpropylcarbamoyl]-2- | |
| methylpropyl](methyl)(aminocarbonyloxy)}methyl)-2-[(S)-2-[(S)-2-(3-{2-[({N-2-[4-(4-amino-1-piperidyl)-3-(6-chloro-1H-1,3- | |
| benzimidazol-2-yl)-5-(3-fluoro-5-tolyl)-2-pyridylamino]ethylcarbamoyl}methyl)-N-methylcarbamoyl]ethoxy}propionylamino)-3- | |
| methylbutyrylamino]propionylamino]phenoxy]-3,4,5-trihydroxytetrahydro-2H-pyran-2-carboxylic acid | |
| 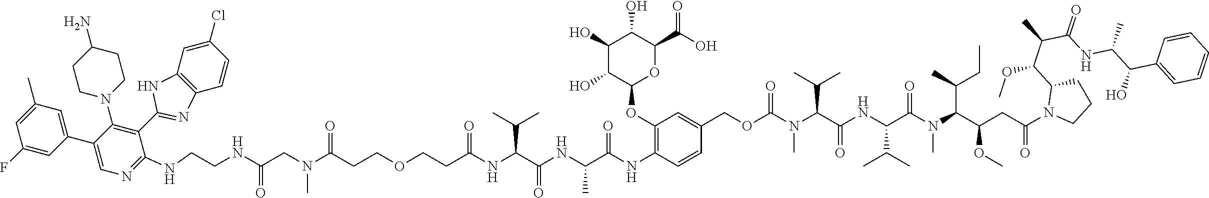 [SMILES: CC[C@H](C)[C@@H]([C@@H](CC(=O)N1CCC[C@H]1[C@H](OC)[C@@H](C)C(=O)N[C@H](C)[C@@H](O)c1ccccc1)OC)N(C)C(=O)[C@@H](NC(=O)[C@H](C(C)C)N(C)C(=O)OCc1ccc(NC(=O)[C@H](C)NC(=O)[C@@H](NC(=O)CCOCCC(=O)N(C)CC(=O)NCCNc2ncc(-c3cc(C)cc(F)c3)c(N3CCC(N)CC3)c2-c2nc3ccc(Cl)cc3[nH]2)C(C)C)c(O[C@@H]2O[C@H](C(=O)O)[C@@H](O)[C@H](O)[C@H]2O)c1)C(C)C] | |
| (Compound 134) | |
| (2S,3S,4S,5R,6S)-6-[5-({[(S)-1-[N-(S)-1-{[(1S,2R)-4-[(S)-2-{(1R,2R)-2-[N-(1R,2S)-2-hydroxy-1-methyl-2-phenylethylcarbamoyl]-1- | 1934.9 |
| methoxypropyl}-1-pyrrolidinyl]-1-[(S)-1-methylpropyl]-2-methoxy-4-oxobutyl]-N-methylcarbamoyl}-2-methylpropylcarbamoyl]-2- | |
| methylpropyl](methyl)(aminocarbonyloxy)}methyl)-2-[(S)-2-[(S)-2-[(R)-3-[N-2-(2-{N-2-[4-(4-amino-1-piperidy])-3-(6-chloro-1H- | |
| 1,3-benzimidazol-2-yl)-5-(3-fluoro-5-toly)-2-pyridylamino]ethylcarbamoyl)ethoxy)ethylcarbamoyl]-3-aminopropionylamino]-3- | |
| methylbutyrylamino]propionylamino]phenoxy]-3,4,5-trihydroxytetrahydro-2H-pyran-2-carboxylic acid | |
| 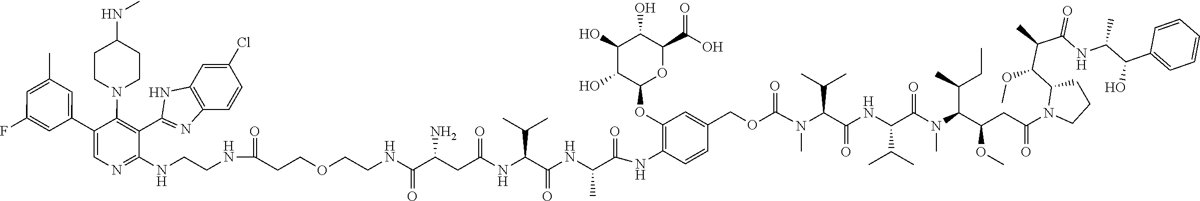 [SMILES: CC[C@H](C)[C@@H]([C@@H](CC(=O)N1CCC[C@H]1[C@H](OC)[C@@H](C)C(=O)N[C@H](C)[C@@H](O)c1ccccc1)OC)N(C)C(=O)[C@@H](NC(=O)[C@H](C(C)C)N(C)C(=O)OCc1ccc(NC(=O)[C@H](C)NC(=O)[C@@H](NC(=O)C[C@@H](N)C(=O)NCCOCCC(=O)NCCNc2ncc(-c3cc(C)cc(F)c3)c(N3CCC(NC)CC3)c2-c2nc3ccc(Cl)cc3[nH]2)C(C)C)c(O[C@@H]2O[C@H](C(=O)O)[C@@H](O)[C@H](O)[C@H]2O)c1)C(C)C] | |
| (Compound 135) | |
| (2S,3S,4S,5R,6S)-6-[5-({[(S)-1-[N-(S)-1-{[(1S,2R)-4-[(S)-2-{(1R,2R)-2-[N-(1R,2S)-2-hydroxy-1-methyl-2-phenylethylcarbamoyl]-1- | 1979.0 |
| methoxypropyl}-1-pyrrolidinyl]-1-[(S)-1-methylpropyl]-2-methoxy-4-oxobutyl]-N-methylcarbamoyl}-2-methylpropylcarbamoyl]-2- | |
| methylpropyl](methyl)(aminocarbonyloxy)}methyl)-2-[(S)-2-[(S)-2-[(R)-3-{N-2-[2-(2-{N-2-[4-(4-amino-1-piperidyl)-3-(6-chloro-1H- | |
| 1,3-benzimidazol-2-yl)-5-(3-fluoro-5-tolyl)-2-pyridylamino]ethylcarbamoyl}ethoxy)ethoxy]ethylcarbamoyl}-3- | |
| aminopropionylamino]-3-methylbutyrylamino]propionylamino]phenoxy]-3,4,5-trihydroxytetrahydro-2H-pyran-2-carboxylic acid | |
| 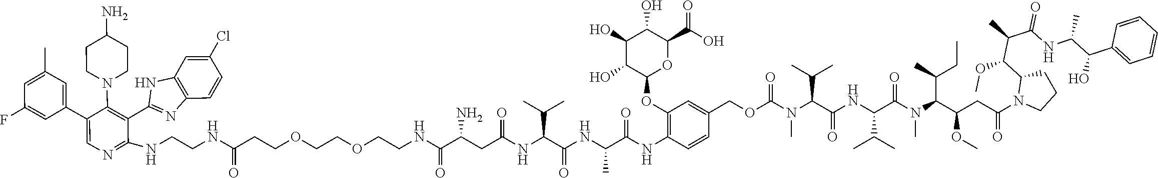 [SMILES: CC[C@H](C)[C@@H]([C@@H](CC(=O)N1CCC[C@H]1[C@H](OC)[C@@H](C)C(=O)N[C@H](C)[C@@H](O)c1ccccc1)OC)N(C)C(=O)[C@@H](NC(=O)[C@H](C(C)C)N(C)C(=O)OCc1ccc(NC(=O)[C@H](C)NC(=O)[C@@H](NC(=O)C[C@@H](N)C(=O)NCCOCCOCCC(=O)NCCNc2ncc(-c3cc(C)cc(F)c3)c(N3CCC(N)CC3)c2-c2nc3ccc(Cl)cc3[nH]2)C(C)C)c(O[C@@H]2O[C@H](C(=O)O)[C@@H](O)[C@H](O)[C@H]2O)c1)C(C)C] | |
| (Compound 136) | |
| (2S,3S,4S,5R,6S)-6-[5-({[(S)-1-[N-(S)-1-{[(1S,2R)-4-[(S)-2-{(1R,2R)-2-[N-(1R,2S)-2-hydroxy-1-methyl-2-phenylethylcarbamoyl]-1- | 2065.0 |
| methoxypropyl}-1-pyrrolidinyl]-1-[(S)-1-methylpropyl]-2-methoxy-4-oxobutyl]-N-methylcarbamoyl}-2-methylpropylcarbamoyl]-2- | |
| methylpropyl](methyl)(aminocarbonyloxy)}methyl)-2-[(S)-2-[(S)-2-[(R)-3-{N-2-[2-(2-{N-2-[4-(4-amino-1-piperidyl)-3-(6-chloro-1H- | |
| 1,3-benzimidazol-2-yl)-5-(3-fluoro-5-tolyl)-2-pyridylamino]ethylcarbamoyl}ethoxy)ethoxy ]ethylcarbamoyl}-3- | |
| aminopropionylamino]-3-methylbutyrylamino]-5-ureidovalerylamino]phenoxy]-3,4,5-trihydroxytetrahydro-2H-pyran-2-carboxylic | |
| acid | |
| 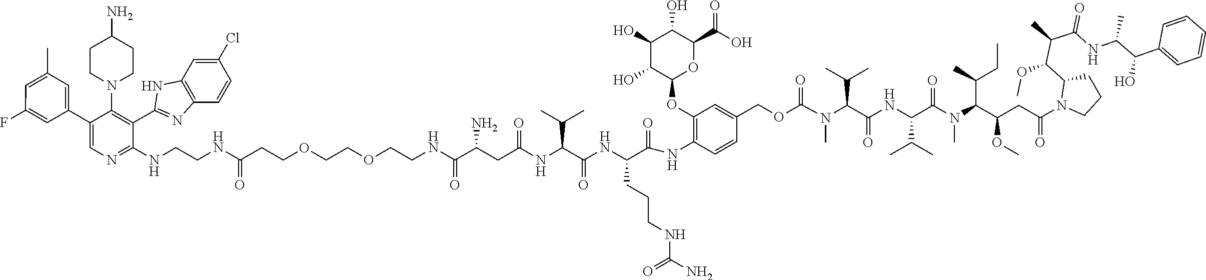 [SMILES: CC[C@H](C)[C@@H]([C@@H](CC(=O)N1CCC[C@H]1[C@H](OC)[C@@H](C)C(=O)N[C@H](C)[C@@H](O)c1ccccc1)OC)N(C)C(=O)[C@@H](NC(=O)[C@H](C(C)C)N(C)C(=O)OCc1ccc(NC(=O)[C@H](CCCNC(N)=O)NC(=O)[C@@H](NC(=O)C[C@@H](N)C(=O)NCCOCCOCCC(=O)NCCNc2ncc(-c3cc(C)cc(F)c3)c(N3CCC(N)CC3)c2-c2nc3ccc(Cl)cc3[nH]2)C(C)C)c(O[C@@H]2O[C@H](C(=O)O)[C@@H](O)[C@H](O)[C@H]2O)c1)C(C)C] | |
| (Compound 137) | |
| (2S,3S,4S,5R,6S)-6-[5-({[(S)-1-[N-(S)-1-{[(1S,2R)-4-[(S)-2-{(1R,2R)-2-[N-(1R,2S)-2-hydroxy-1-methyl-2-phenylethylcarbamoyl]-1- | 1832.9 |
| methoxypropyl}-1-pyrrolidinyl]-1-[(S)-1-methylpropyl]-2-methoxy-4-oxobutyl]-N-methylcarbamoyl}-2-methylpropylcarbamoyl]-2- | |
| methylpropyl](methyl)(aminocarbonyloxy)}methyl)-2-[(S)-2-[(S)-2-[3-(2-{N-2-[4-(4-amino-1-piperidyl)-3-(6-fluoro-1H-1,3- | |
| benzimidazol-2-yl)-5-(3-fluoro-5-tolyl)-2-pyridylamino]ethylcarbamoyl}ethoxy)propionylamino]-3- | |
| methylbutyrylamino]propionylamino]phenoxy]-3,4,5-trihydroxytetrahydro-2H-pyran-2-carboxylic acid | |
| 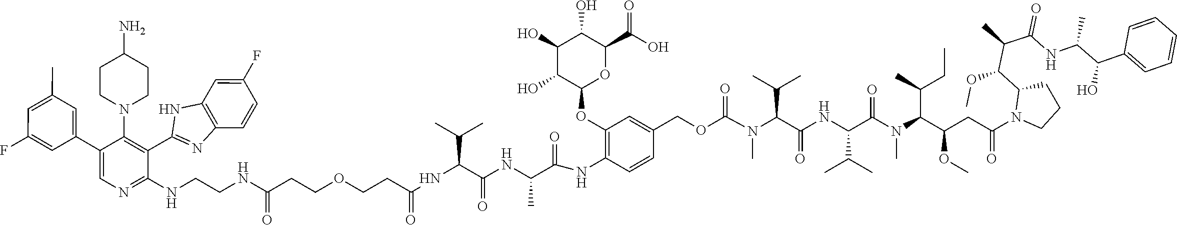 [SMILES: CC[C@H](C)[C@@H]([C@@H](CC(=O)N1CCC[C@H]1[C@H](OC)[C@@H](C)C(=O)N[C@H](C)[C@@H](O)c1ccccc1)OC)N(C)C(=O)[C@@H](NC(=O)[C@H](C(C)C)N(C)C(=O)OCc1ccc(NC(=O)[C@H](C)NC(=O)[C@@H](NC(=O)CCOCCC(=O)NCCNc2ncc(-c3cc(C)cc(F)c3)c(N3CCC(N)CC3)c2-c2nc3ccc(F)cc3[nH]2)C(C)C)c(O[C@@H]2O[C@H](C(=O)O)[C@@H](O)[C@H](O)[C@H]2O)c1)C(C)C] | |
| (Compound 138) | |
| (2S,3S,4S,5R,6S)-6-[5-({[(S)-1-[N-(S)-1-{[(1S,2R)-4-[(S)-2-{(1R,2R)-2-[N-(1R,2S)-2-hydroxy-1-methyl-2-phenylethylcarbamoyl]-1- | 1864.9 |
| methoxypropyl}-1-pyrrolidiny1]-1-[(S)-1-methylpropyl]-2-methoxy-4-oxobutyl]-N-methylcarbamoyl}-2-methylpropylcarbamoyl]-2- | |
| methylpropyl](methyl)(aminocarbonyloxy)}methyl)-2-[(S)-2-[(S)-2-[3-(2-{N-2-[4-(4-amino-1-piperidyl)-3-(6-chloro-1H-1,3- | |
| benzimidazol-2-yl)-5-(3-fluoro-5-methoxyphenyl)-2-pyridylamino]ethylcarbamoy]}ethoxy)propionylamino]-3- | |
| methylbutyrylamino\|propionylamino]phenoxy]-3,4,5-trihydroxytetrahydro-2H-pyran-2-carboxylic acid | |
| 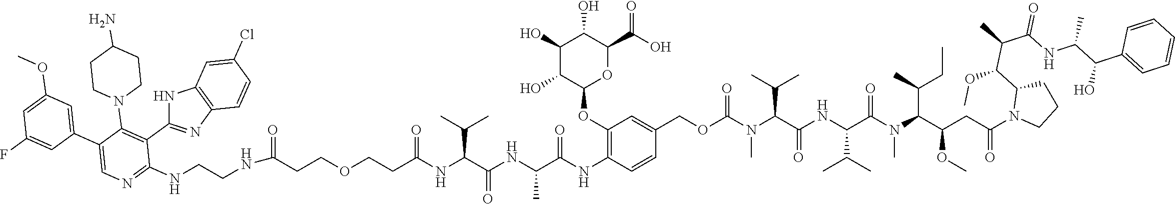 [SMILES: CC[C@H](C)[C@@H]([C@@H](CC(=O)N1CCC[C@H]1[C@H](OC)[C@@H](C)C(=O)N[C@H](C)[C@@H](O)c1ccccc1)OC)N(C)C(=O)[C@@H](NC(=O)[C@H](C(C)C)N(C)C(=O)OCc1ccc(NC(=O)[C@H](C)NC(=O)[C@@H](NC(=O)CCOCCC(=O)NCCNc2ncc(-c3cc(F)cc(OC)c3)c(N3CCC(N)CC3)c2-c2nc3ccc(Cl)cc3[nH]2)C(C)C)c(O[C@@H]2O[C@H](C(=O)O)[C@@H](O)[C@H](O)[C@H]2O)c1)C(C)C] | |
| (Compound 139) | |
| (2S,3S,4S,5R,6S)-6-[5-({[(S)-1-[N-(S)-1-{[(1S,2R)-4-[(S)-2-{(1R,2R)-2-[N-(1R,2S)-2-hydroxy-1-methyl-2-phenylethylcarbamoyl]-1- | 1862.8 |
| methoxypropyl}-1-pyrrolidinyl]-1-[(S)-1-methylpropyl]-2-methoxy-4-oxobutyl]-N-methylcarbamoyl}-2-methylpropylcarbamoyl]-2- | |
| methylpropyl](methyl)(aminocarbonyloxy)}methyl)-2-[(S)-2-[(S)-2-[3-(2-{N-2-[4-(4-amino-1-piperidyl)-3-(5-fluoro-7-methoxy-1H- | |
| 1,3-benzimidazol-2-yl)-5-(3-fluoro-5-tolyl)-2-pyridylamino]ethylcarbamoyl}ethoxy)propionylamino]-3- | |
| methylbutyrylamino]propionylamino]phenoxy]-3,4,5-trihydroxytetrahydro-2H-pyran-2-carboxylic acid | |
| 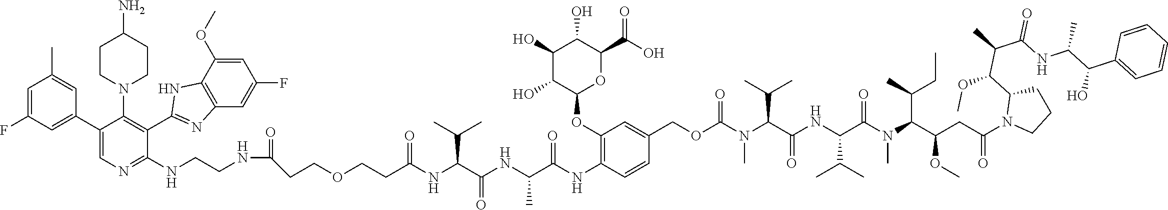 [SMILES: CC[C@H](C)[C@@H]([C@@H](CC(=O)N1CCC[C@H]1[C@H](OC)[C@@H](C)C(=O)N[C@H](C)[C@@H](O)c1ccccc1)OC)N(C)C(=O)[C@@H](NC(=O)[C@H](C(C)C)N(C)C(=O)OCc1ccc(NC(=O)[C@H](C)NC(=O)[C@@H](NC(=O)CCOCCC(=O)NCCNc2ncc(-c3cc(C)cc(F)c3)c(N3CCC(N)CC3)c2-c2nc3cc(F)cc(OC)c3[nH]2)C(C)C)c(O[C@@H]2O[C@H](C(=O)O)[C@@H](O)[C@H](O)[C@H]2O)c1)C(C)C] | |
| (Compound 140) | |
| (2S,3S,4S,5R,6S)-6-[5-({[(S)-1-[N-(S)-1-{[(1S,2R)-4-[(S)-2-{(1R,2R)-2-[N-(1R,2S)-2-hydroxy-1-methyl-2-phenylethylcarbamoyl]-1- | 1943.9 |
| methoxypropyl}-1-pyrrolidinyl]-1-[(S)-1-methylpropyl]-2-methoxy-4-oxobutyl]-N-methylcarbamoyl}-2-methylpropylcarbamoyl]-2- | |
| methylpropyl](methyl)(aminocarbonyloxy)}methyl)-2-[(S)-2-[(S)-2-(3-{2-[4-(2-{N-2-[4-(4-amino-1-piperidyl)-3-(6-chloro-1H-1,3- | |
| benzimidazol-2-yl)-5-(3-fluoro-5-tolyl)-2-pyridylamino]ethylcarbamoyl}ethyl)-1H-1,2,3-triazol-1-yl]ethoxy}propionylamino)-3- | |
| methylbutyrylamino]propionylamino]phenoxy]-3,4,5-trihydroxytetrahydro-2H-pyran-2-carboxylic acid | |
| 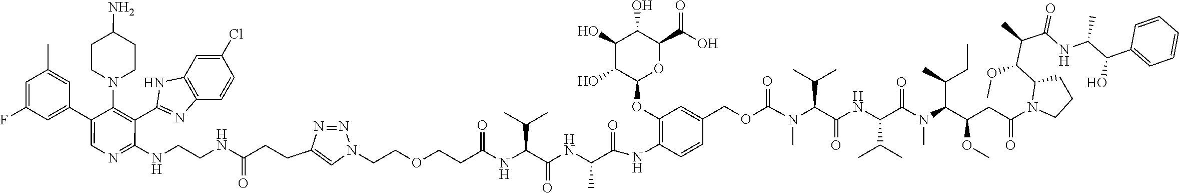 [SMILES: CC[C@H](C)[C@@H]([C@@H](CC(=O)N1CCC[C@H]1[C@H](OC)[C@@H](C)C(=O)N[C@H](C)[C@@H](O)c1ccccc1)OC)N(C)C(=O)[C@@H](NC(=O)[C@H](C(C)C)N(C)C(=O)OCc1ccc(NC(=O)[C@H](C)NC(=O)[C@@H](NC(=O)CCOCCn2cc(CCC(=O)NCCNc3ncc(-c4cc(C)cc(F)c4)c(N4CCC(N)CC4)c3-c3nc4ccc(Cl)cc4[nH]3)nn2)C(C)C)c(O[C@@H]2O[C@H](C(=O)O)[C@@H](O)[C@H](O)[C@H]2O)c1)C(C)C] | |
| (Compound 141) | |
| (2S,3S,4S,5R,6S)-6-[5-({[(S)-1-[N-(S)-1-{[(1S,2R)-4-[(S)-2-{(1R,2R)-2-[N-(1R,2S)-2-hydroxy-1-methyl-2-phenylethylcarbamoyl]-1- | 1848.9 |
| methoxypropyl}-1-pyrrolidiny1]-1-[(S)-1-methylpropyl]-2-methoxy-4-oxobutyl]-N-methylcarbamoyl}-2-methylpropylcarbamoyl]-2- | |
| methylpropyl](methyl)(aminocarbonyloxy)}methyl)-2-[(S)-2-[(S)-2-[3-(2-{3-[4-(4-amino-1-piperidyl)-3-(6-chloro-1H-1,3- | |
| benzimidazol-2-yl)-5-(3-fluoro-5-tolyl)-2-pyridylamino]propionylamino}ethoxy)propionylamino]-3- | |
| methylbutyrylamino]propionylamino]phenoxy]-3,4,5-trihydroxytetrahydro-2H-pyran-2-carboxylic acid | |
| 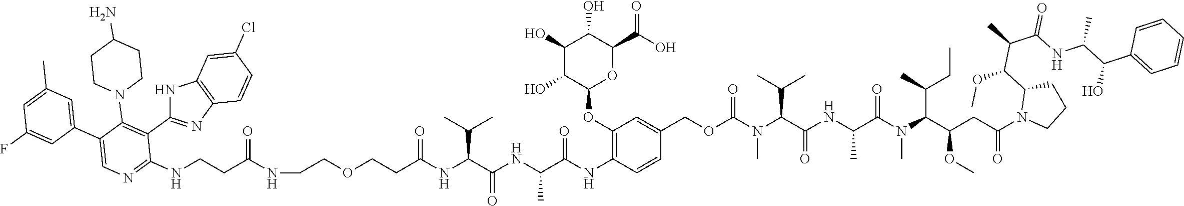 [SMILES: CC[C@H](C)[C@@H]([C@@H](CC(=O)N1CCC[C@H]1[C@H](OC)[C@@H](C)C(=O)N[C@H](C)[C@@H](O)c1ccccc1)OC)N(C)C(=O)[C@H](C)NC(=O)[C@H](C(C)C)N(C)C(=O)OCc1ccc(NC(=O)[C@H](C)NC(=O)[C@@H](NC(=O)CCOCCNC(=O)CCNc2ncc(-c3cc(C)cc(F)c3)c(N3CCC(N)CC3)c2-c2nc3ccc(Cl)cc3[nH]2)C(C)C)c(O[C@@H]2O[C@H](C(=O)O)[C@@H](O)[C@H](O)[C@H]2O)c1] | |
| (Compound 194) | |
| (2S,3S,4S,5R,6S)-6-[5-({[(S)-1-[N-(S)-1-{[(1S,2R)-4-[(S)-2-{(1R,2R)-2-[N-(1R,2S)-2-hydroxy-1-methyl-2-phenylethylcarbamoyl]-1- | 1949.0 |
| methoxypropyl}-1-pyrrolidiny1]-1-[(S)-1-methylpropyl]-2-methoxy-4-oxobutyl]-N-methylcarbamoyl}-2-methylpropylcarbamoyl]-2- | |
| methylpropyl](methyl)(aminocarbonyloxy)}methyl)-2-[(S)-2-[(S)-2-[3-(2-{N-3-[4-(4-amino-1-piperidyl)-3-(6-chloro-1H-1,3- | |
| benzimidazol-2-yl)-5-(3-fluoro-5-tolyl)-2-pyridylamino]propylcarbamoyl}ethoxy)propionylamino]-3-methylbutyrylamino]-5- | |
| ureidovalerylamino]phenoxy ]-3,4,5-trihydroxytetrahydro-2H-pyran-2-carboxylic acid | |
| 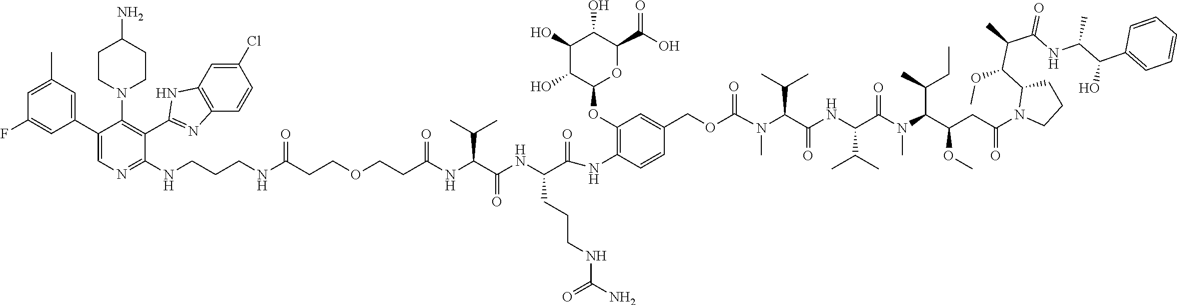 [SMILES: CC[C@H](C)[C@@H]([C@@H](CC(=O)N1CCC[C@H]1[C@H](OC)[C@@H](C)C(=O)N[C@H](C)[C@@H](O)c1ccccc1)OC)N(C)C(=O)[C@@H](NC(=O)[C@H](C(C)C)N(C)C(=O)OCc1ccc(NC(=O)[C@H](CCCNC(N)=O)NC(=O)[C@@H](NC(=O)CCOCCC(=O)NCCCNc2ncc(-c3cc(C)cc(F)c3)c(N3CCC(N)CC3)c2-c2nc3ccc(Cl)cc3[nH]2)C(C)C)c(O[C@@H]2O[C@H](C(=O)O)[C@@H](O)[C@H](O)[C@H]2O)c1)C(C)C] | |
| (Compound 195) | |
| (2S,3S,4S,5R,6S)-3,4,5-trihydroxy-6-{2-[(S)-2-[(S)-2-[(S)-5-hydroxy-2-[3-(2-{N-2-[4-(4-amino-1-piperidyl)-3-(6-chloro-1H-1,3- | 1964.0 |
| benzimidazol-2-yl)-5-(3-fluoro-5-tolyl)-2-pyridylamino]ethylcarbamoyl}ethoxy)propionylamino]valerylamino]-3- | |
| methylbutyrylamino]propionylamino]-5-({methyl[(S)-2-methyl-1-[N-(S)-1-{[(1S,2R)-2-methoxy-4-[(S)-2-[(1R,2R)-1-methoxy-2-[N- | |
| (1R,2S)-2-hydroxy-1-methyl-2-phenylethylcarbamoyl\|propyl]-1-pyrrolidinyl]-1-[(S)-1-methylpropyl]-4-oxobutyl]-N- | |
| methylcarbamoyl}-2-methylpropylcarbamoyl]propyl](aminocarbonyloxy)}methyl)phenoxy}tetrahydro-2H-pyran-2-carboxylic acid | |
| 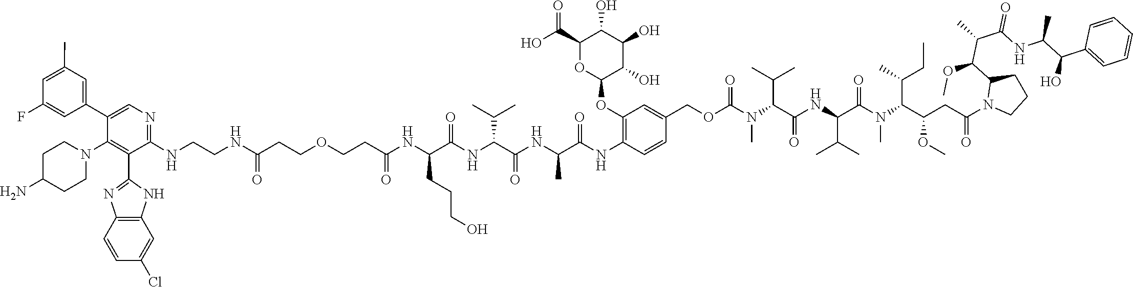 | |
| (Compound 239) | |
| (2S,3S,4S,5R,6S)-3,4,5-trihydroxy-6-[5-({methyl[(S)-2-methyl-1-[N-(S)-1-{[(1S,2R)-2-methoxy-4-[(S)-2-{(1R,2R)-1-methoxy-2-[N- | 1848.9 |
| (1R,2S)-2-hydroxy-1-methyl-2-phenylethylcarbamoyl]propyl]-1-pyrrolidinyl]-1-[(S)-1-methylpropyl]-4-oxobutyl]-N- | |
| methylcarbamoyl}-2-methylpropylcarbamoyl]propyl](aminocarbonyloxy)}methyl)-2-[(R)-2-[(S)-3-methyl-2-[3-(2-{N-2-[4-(4-amino- | |
| 1-piperidyl)-3-(6-chloro-1H-1,3-benzimidazol-2-yl)-5-(3-fluoro-5-tolyl)-2- | |
| pyridylamino]ethylcarbamoyl}ethoxy)propionylamino]butyrylamino]propionylamino]phenoxy]tetrahydro-2H-pyran-2-carboxylic acid | |
| 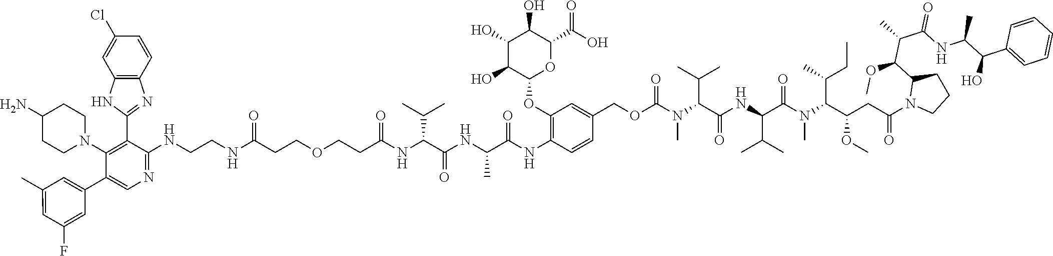 | |
| (Compound 240) | |
| (2S,3S,4S,5R,6S)-3,4,5-trihydroxy-6-[5-({methyl[(S)-2-methyl-1-[N-(S)-1-{[(1S,2R)-2-methoxy-4-[(S)-2-[(1R,2R)-1-methoxy-2-[N- | 1934.9 |
| (1R,2S)-2-hydroxy-1-methyl-2-phenylethylcarbamoyl]propyl]-1-pyrrolidinyl]-1-[(S)-1-methylpropyl]-4-oxobutyl]-N- | |
| methylcarbamoyl}-2-methylpropylcarbamoyl]propyl](aminocarbonyloxy)}methyl)-2-[(R)-2-[(S)-3-methyl-2-[3-(2-{N-2-[4-(4-amino- | |
| 1-piperidyl)-3-(6-chloro-1H-1,3-benzimidazol-2-yl)-5-(3-fluoro-5-toly])-2- | |
| pyridylamino]ethylcarbamoyl}ethoxy)propionylamino]butyrylamino]-5-ureidovalerylamino]phenoxy ]tetrahydro-2H-pyran-2- | |
| carboxylic acid | |
| 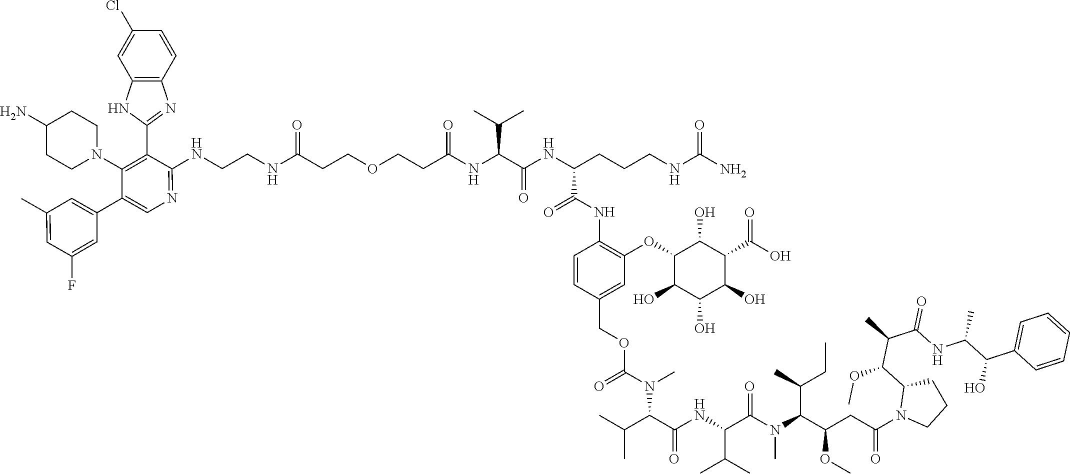 [SMILES: CC[C@H](C)[C@@H]([C@@H](CC(=O)N1CCC[C@H]1[C@H](OC)[C@@H](C)C(=O)N[C@H](C)[C@@H](O)c1ccccc1)OC)N(C)C(=O)[C@@H](NC(=O)[C@H](C(C)C)N(C)C(=O)OCc1ccc(NC(=O)[C@@H](CCCNC(N)=O)NC(=O)[C@@H](NC(=O)CCOCCC(=O)NCCNc2ncc(-c3cc(C)cc(F)c3)c(N3CCC(N)CC3)c2-c2nc3ccc(Cl)cc3[nH]2)C(C)C)c(O[C@H]2[C@H](O)[C@@H](O)[C@H](O)[C@@H](C(=O)O)[C@H]2O)c1)C(C)C] | |
| (Compound 241) | |
| (2S,3S,4S,5R,6S)-3,4,5-trihydroxy-6-[5-({methyl[(S)-2-methyl-1-[N-(S)-1-{[(1S,2R)-2-methoxy-4-[(S)-2-[(1R,2R)-1-methoxy-2-[N- | 1932.9 |
| (1R,2S)-2-hydroxy-1-methyl-2-phenylethylcarbamoyl]propyl]-1-pyrrolidiny1]-1-[(S)-1-methylpropyl]-4-oxobutyl]-N- | |
| methylcarbamoyl}-2-methylpropylcarbamoyl]propyl](aminocarbonyloxy)}methyl)-2-[(S)-2-{1-[3-(2- {N-2-[4-(4-amino-1-piperidyl)-3- | |
| (6-chloro-1H-1,3-benzimidazol-2-yl)-5-(3-fluoro-5-tolyl)-2- | |
| pyridylamino]ethylcarbamoyl}ethoxy )propionylamino]cyclovalerylamino}-5-ureidovalerylaminophenoxy]tetrahydro-2H-pyran-2- | |
| carboxylic acid | |
| 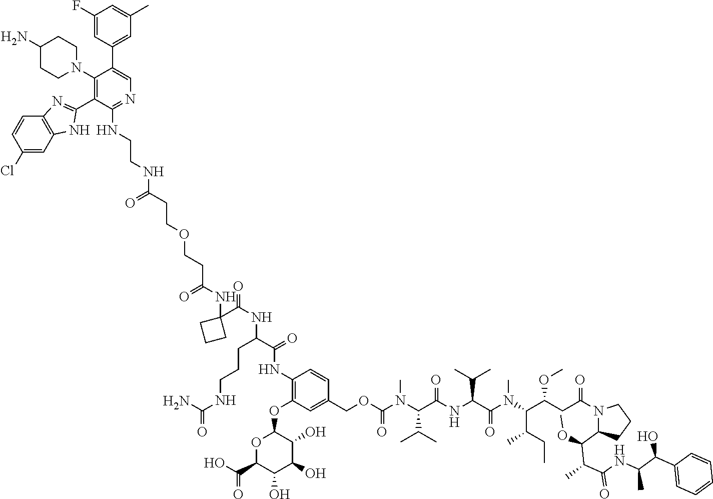 [SMILES: CC[C@H](C)[C@@H]([C@@H](CC(=O)N1CCC[C@H]1[C@H](OC)[C@@H](C)C(=O)N[C@H](C)[C@@H](O)c1ccccc1)OC)N(C)C(=O)[C@@H](NC(=O)[C@H](C(C)C)N(C)C(=O)OCc1ccc(NC(=O)C(CCCNC(N)=O)NC(=O)C2(NC(=O)CCOCCC(=O)NCCNc3ncc(-c4cc(C)cc(F)c4)c(N4CCC(N)CC4)c3-c3nc4ccc(Cl)cc4[nH]3)CCC2)c(O[C@@H]2O[C@H](C(=O)O)[C@@H](O)[C@H](O)[C@H]2O)c1)C(C)C] | |
| (Compound 242) | |
| (2S,3S,4S,5R,6S)-3,4,5-trihydroxy-6-{2-[(S)-2-[(S)-2-[(S)-3-(4-imidazolyl)-2-3-(2-{N-2-[4-(4-amino-1-piperidyl)-3-(6-chloro-1H- | 2072.0 |
| 1,3-benzimidazol-2-yl)-5-(3-fluoro-5-tolyl)-2-pyridylamino]ethylcarbamoyl}ethoxy)propionylamino]propionylamino]-3- | |
| methylbutyrylamino]-5-ureidovalerylamino]-5-({methyl[(S)-2-methyl-1-[N-(S)-1-{[(1S,2R)-2-methoxy-4-[(S)-2-[(IR,2R)-1-methoxy- | |
| 2-[N-(1R,2S)-2-hydroxy-1-methyl-2-phenylethylcarbamoyl]propyl]-1-pyrrolidinyl]-1-[(S)-1-methylpropyl]-4-oxobutyl]-N- | |
| methylcarbamoyl}-2-methylpropylcarbamoyl]propyl](aminocarbonyloxy)}methyl)phenoxy}tetrahydro-2H-pyran-2-carboxylic acid | |
| 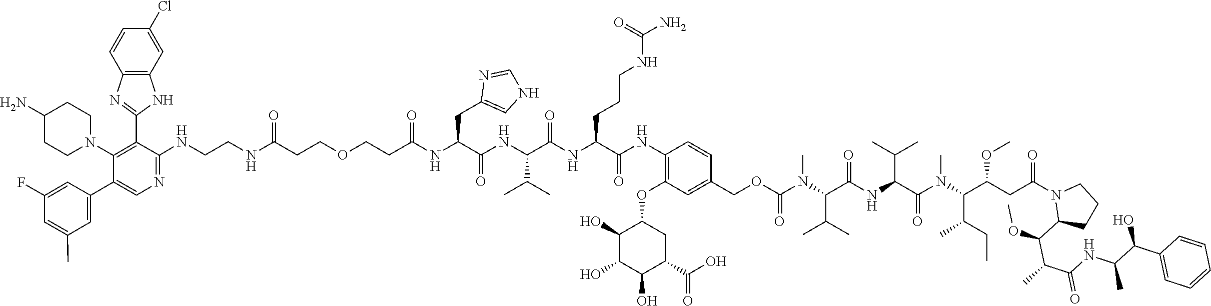 [SMILES: CC[C@H](C)[C@@H]([C@@H](CC(=O)N1CCC[C@H]1[C@H](OC)[C@@H](C)C(=O)N[C@H](C)[C@@H](O)c1ccccc1)OC)N(C)C(=O)[C@@H](NC(=O)[C@H](C(C)C)N(C)C(=O)OCc1ccc(NC(=O)[C@H](CCCNC(N)=O)NC(=O)[C@@H](NC(=O)[C@H](Cc2c[nH]cn2)NC(=O)CCOCCC(=O)NCCNc2ncc(-c3cc(C)cc(F)c3)c(N3CCC(N)CC3)c2-c2nc3ccc(Cl)cc3[nH]2)C(C)C)c(O[C@@H]2C[C@H](C(=O)O)[C@@H](O)[C@H](O)[C@H]2O)c1)C(C)C] | |
| (Compound 243) | |
| (2S,3S,4S,5R,6S)-3,4,5-trihydroxy-6-[4-({methyl[(S)-2-methyl-1-[N-(S)-1-{[(1S,2R)-2-methoxy-4-[(S)-2-{(1R,2R)-1-methoxy-2-[N- | 1934.9 |
| (1R,2S)-2-hydroxy-1-methyl-2-phenylethylcarbamoyl\|propyl]-1-pyrrolidinyl]-1-[(S)-1-methylpropyl]-4-oxobutyl]-N- | |
| methylcarbamoyl}-2-methylpropylcarbamoyl]propyl](aminocarbonyloxy)}methyl)-2-[(S)-2-[(S)-3-methyl-2-[3-(2-{N-2-[4-(4-amino- | |
| 1-piperidyl)-3-(6-chloro-1H-1,3-benzimidazol-2-yl)-5-(3-fluoro-5-tolyl)-2- | |
| pyridylamino]ethylcarbamoyl}ethoxy)propionylamino]butyrylamino]-5-ureidovalerylamino]phenoxy]tetrahydro-2H-pyran-2- | |
| carboxylic acid | |
| 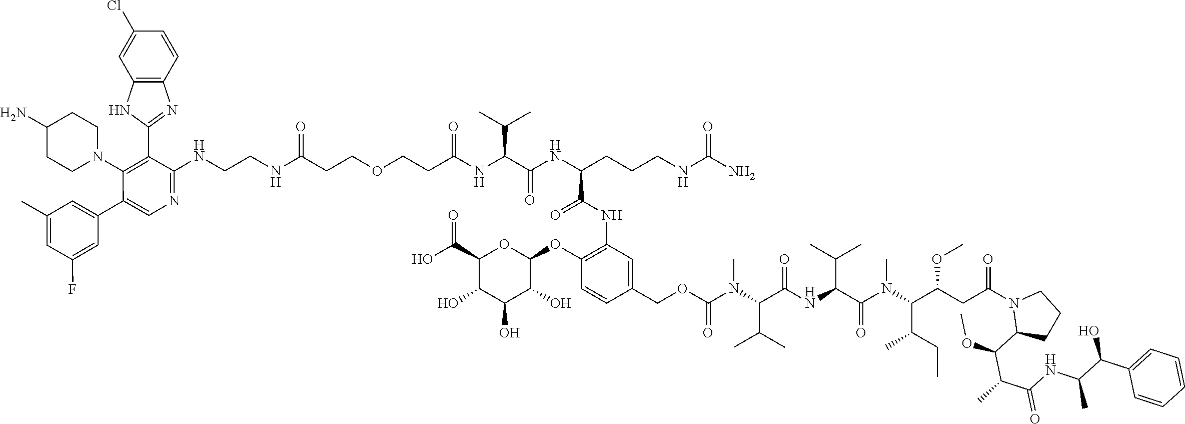 [SMILES: CC[C@H](C)[C@@H]([C@@H](CC(=O)N1CCC[C@H]1[C@H](OC)[C@@H](C)C(=O)N[C@H](C)[C@@H](O)c1ccccc1)OC)N(C)C(=O)[C@@H](NC(=O)[C@H](C(C)C)N(C)C(=O)OCc1ccc(O[C@@H]2O[C@H](C(=O)O)[C@@H](O)[C@H](O)[C@H]2O)c(NC(=O)[C@H](CCCNC(N)=O)NC(=O)[C@@H](NC(=O)CCOCCC(=O)NCCNc2ncc(-c3cc(C)cc(F)c3)c(N3CCC(N)CC3)c2-c2nc3ccc(Cl)cc3[nH]2)C(C)C)c1)C(C)C] | |
| (Compound 248) | |
| (2S,3S,4S,5R,6S)-3,4,5-trihydroxy-6-[4-({methyl[(S)-2-methyl-1-[N-(S)-1-{[(1S,2R)-2-methoxy-4-[(S)-2-[(1R,2R)-1-methoxy-2-[N- | 1848.9 |
| (1R,2S)-2-hydroxy-1-methyl-2-phenylethylcarbamoyl]propyl]-1-pyrrolidinyl]-1-[(S)-1-methylpropyl]-4-oxobutyl]-N- | |
| methylcarbamoyl}-2-methylpropylcarbamoyl]propyl](aminocarbonyloxy)}methyl)-2-[(S)-2-[(S)-3-methyl-2-[3-(2-{N-2-[4-(4-amino- | |
| 1-piperidyl)-3-(6-chloro-1H-1,3-benzimidazol-2-yl)-5-(3-fluoro-5-tolyl)-2- | |
| pyridylamino]ethylcarbamoyl}ethoxy)propionylamino]butyrylamino]propionylamino]phenoxy]tetrahydro-2H-pyran-2-carboxylic acid | |
| 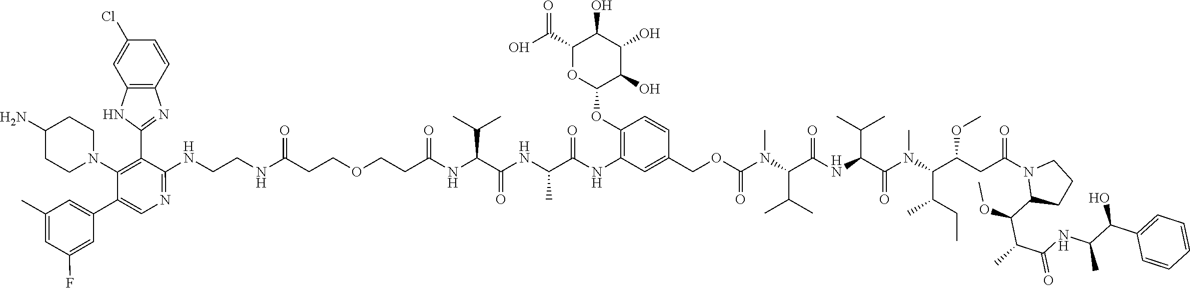 | |
| (Compound 249) | |

### Example E. 1-(S)-1-[N—(S)-1-{[(1S,2R)-4-[(S)-2-{(2R)-2-[N-(1R,2S)-2-hydroxy-1-methyl-2-phenylethylcarbamoyl]-1-methoxypropyl}-1-pyrrolidinyl]-1-[(S)-1-methylpropyl]-2-methoxy-4-oxobutyl]-N-methylcarbamoyl}-2-methylpropylcarbamoyl]-2-methylpropyl 1-methyl {p-[(S)-2-[(S)-2-{1-[2-(2-{N-2-[4-(4-amino-1-piperidyl)-3-(6-chloro-1H-1,3-benzimidazol-2-yl)-5-(3-fluoro-5-tolyl)-2-pyridylamino]ethylcarbamoyl}ethoxy)ethyl]-1H-1,2,3-triazol-4-ylcarbonylamino}-3-methylbutyrylamino]-5-ureidovalenylamino]phenyl}methanecarbamate (Compound 142)

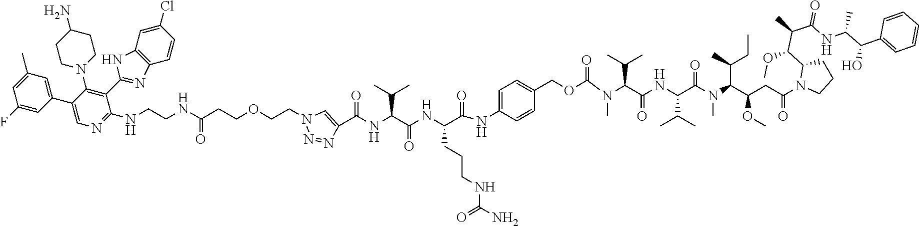 [SMILES: CC[C@H](C)[C@@H]([C@@H](CC(=O)N1CCC[C@H]1[C@H](OC)[C@@H](C)C(=O)N[C@H](C)[C@@H](O)c1ccccc1)OC)N(C)C(=O)[C@@H](NC(=O)[C@H](C(C)C)N(C)C(=O)OCc1ccc(NC(=O)[C@H](CCCNC(N)=O)NC(=O)[C@@H](NC(=O)c2cn(CCOCCC(=O)NCCNc3ncc(-c4cc(C)cc(F)c4)c(N4CCC(N)CC4)c3-c3nc4ccc(Cl)cc4[nH]3)nn2)C(C)C)cc1)C(C)C]

Step E-1, preparation of tert-butyl (1-(2-((2-(3-(2-bromoethoxy)propanamido)ethyl)amino)-3-(6-chloro-1H-benzo[d]imidazol-2-yl)-5-(3-fluoro-5-methylphenyl)pyridin-4-yl)piperidin-4-yl)carbamate: Into a 40-mL vial was placed a mixture of 3-(2-bromoethoxy)propanoic acid (200 mg, 1.51 Eq, 1.02 mmol). HATU (310 mg, 1.21 Eq, 815 μmol), DIEA (270 mg, 364 LL, 3.10 Eq, 2.09 mmol) and DMF (4 mL). The reaction mixture was stirred at 25° C. for 20 minutes, then tert-butyl (1-(2-((2-aminoethyl)amino)-3-(6-chloro-1H-benzo[d]imidazol-2-yl)-5-(3-fluoro-5-methylphenyl)pyridin-4-yl)piperidin-4-yl)carbamate (400 mg, 1 Eq, 673 μmol) was added and the reaction mixture was stirred at 25° C. for 1 hour. The crude product was purified by Prep-HPLC with the following conditions: Column: SunFire prep OBD 19*150 mm 5 um; Mobile Phase A: Water (0.05% formic acid): Mobile Phase B: ACN; Gradient; 20% B to 58% B in 8 min; Flow rate: 20 mL/min; Wave Length: 254 nm. The collected fractions were dried by lyophilization to provide tert-butyl (1-(2-((2-(3-(2-bromoethoxy)propanamido)ethyl)amino)-3-(6-chloro-1H-benzo[d]imidazol-2-yl)-5-(3-fluoro-5-methylphenyl)pyridin-4-yl)piperidin-4-yl)carbamate (220 mg, 285 μmol, 42.3%); (M+H)⁺=772.1.

Step E-2, preparation of tert-butyl (1(2-((2-(3-(2-azidoethoxy)propanamido)ethyl)amino)-3-(6-chloro-1H-benzo[d]imidazol-2-yl)-5-(3-fluoro-5-methylphenyl)pyridin-4-yl)piperidin-4-yl)carbamate: Into a 40-mL vial was placed a mixture of tert-butyl (1-(2-((2-(3-(2-bromoethoxy)propanamido)ethyl)amino)-3-(6-chloro-1H-benzo[d]imidazol-2-yl)-5-(3-fluoro-5-methylphenyl)pyridin-4-yl)piperidin-4-yl)carbamate (200 mg, 1 Eq, 259 μmol), sodium azide (170 mg, 10.1 Eq, 2.61 mmol) and DMF (2 mL). The reaction mixture was stirred at 110° C. for 3 hours. The mixture was quenched by adding saturated NaHSO₃ solution, diluted with 50 mL of water, extracted with EtOAc (50 mL×3), the combined organic layers were washed with water (50 mL×2) and brine (50 mL), dried over anhydrous sodium sulfate and concentrated under reduced pressure to provide tert-butyl (1-(2-((2-(3-(2-azidoethoxy)propanamido)ethyl)amino)-3-(6-chloro-1H-benzo[d]imidazol-2-yl)-5-(3-fluoro-5-methylphenyl)pyridin-4-yl)piperidin-4-yl)carbamate (110 mg, 150 μmol, 57.8%); (M+H)⁺=735.3.

Step E-3, preparation of 1-(2-(3-((2-((4-(4-((tert-butoxycarbonyl)amino)piperidin-1-yl)-3-(6-chloro-1H-benzo[d]imidazol-2-yl)-5-(3-fluoro-5-methylphenyl)pyridin-2-yl)amino)ethyl)amino)-3-oxopropoxy)ethyl)-1H-1,2,3-triazole-4-carboxylic acid: Into a 40-mL vial was placed tert-butyl (1-(2-((2-(3-(2-azidoethoxy)propanamido)ethyl)amino)-3-(6-chloro-1H-benzo[d]imidazol-2-yl)-5-(3-fluoro-5-methylphenyl)pyridin-4-yl)piperidin-4-yl)carbamate (110 mg, 1 Eq, 150 μmol), isoascorbic acid (6 mg, 4 μL, 0.2 Eq, 0.03 mmol), propiolic acid (15 mg, 1.4 Eq, 0.21 mmol) and copper (II) sulfate anhydrous (3 mg, 1 μL, 0.1 Eq. 0.02 mmol) in ethanol (1.1 mL). The resulting reaction mixture was stirred for 1 hour at 25° C. The crude product was purified by Prep-HPLC with the following conditions: Column: SunFire prep OBD 19*150 mm 5 um; Mobile Phase A: Water (0.05% trifluoroacetic acid); Mobile Phase B: ACN; Gradient: 25% B to 65% B in 8 min; Flow rate: 20 mL/min; Wave Length: 254 nm. The collected fractions were dried by lyophilization to provide 1-(2-(3-((2-((4-(4-((tert-butoxy carbonyl)amino)piperidin-1-yl)-3-(6-chloro-1H-benzo[d]imidazol-2-yl)-5-(3-fluoro-5-methylphenyl)pyridin-2-yl)amino)ethyl)amino)-3-oxopropoxy)ethyl)-1H-1,2,3-triazole-4-carboxylic acid (80 mg, 99 μmol, 66%); (M+H)⁺=805.4.

Step E-4, preparation of 4-((S)-2-((S)-2-(1-(2-(3-((24(4-(4-((tert-butoxycarbonyl)amino)piperidin-1-yl)-3-(6-chloro-1H-benzo[d]imidazol-2-yl)-5-(3-fluoro-5-methylphenyl)pyridin-2-yl)amino)ethyl)amino)-3-oxopropoxy)ethyl)-1H-1,2,3-triazole-4-carboxamido)-3-methylbutanamido)-5-ureidopentanamido)benzyl ((S)-1-(((S)-1-(((3R,4S,5S)-1-((S)-2-((1R,2R)-3-(((1S,2R)-1-hydroxy-1-phenylpropan-2-yl)amino)-1-methoxy-2-methyl-3-oxopropyl)pyrrolidin-1-yl)-3-methoxy-5-methyl-1-oxoheptan-4-yl)(methyl)amino)-3-methyl-1-oxobutan-2-yl)amino)-3-methyl-1-oxobutan-2-yl)(methyl)carbamate: Into a 40-mL vial was placed 1-(2-(3-((2-((4-(4-((tert-butoxycarbonyl)amino)piperidin-1-yl)-3-(6-chloro-1H-benzo[d]imidazol-2-yl)-5-(3-fluoro-5-methylphenyl)pyridin-2-yl)amino)ethyl)amino)-3-oxopropoxy)ethyl)-1H-1,2,3-triazole-4-carboxylic acid (40 mg, 1 Eq, 50 μmol), 4-((S)-2-((S)-2-amino-3-methylbutanamido)-5-ureidopentanamido)benzyl ((S)-1-(((S)-1-(((3R,4S,5S)-1-((S)-2-((1R,2R)-3-(((1S,2R)-1-hydroxy-1-phenylpropan-2-yl)amino)-1-methoxy-2-methyl-3-oxopropyl)pyrrolidin-1-yl)-3-methoxy-5-methyl-1-oxoheptan-4-yl)(methyl)amino)-3-methyl-1-oxobutan-2-yl)amino)-3-methyl-1-oxobutan-2-yl)(methyl)carbamate (55 mg, 0.99 Eq, 49 μmol), pentafluorophenyldiphenylphosphinate (30 mg, 1.6 Eq, 78 μmol) and 4-methylmorfolin (15 mg, 16 μL, 3.0 Eq, 0.15 mmol) in DMF (0.4 mL). The resulting reaction mixture was stirred for 2 hours at 25° C. The crude product was purified by Prep-HPLC with the following conditions: Column: SunFire prep OBD 19*150 mm 5 um; Mobile Phase A: Water (0.05% trifluoracetic acid); Mobile Phase B: ACN; Gradient: 25% B to 65% B in 8 min; Flow rate: 20 mL/min; Wave Length: 254 nm. The collected fractions were dried by lyophilization to provide 4-((S)-2-((S)-2-(1-(2-(3-((2-((4-(4-((tert-butoxycarbonyl)amino)piperidin-1-yl)-3-(6-chloro-1H-benzo[d]imidazol-2-yl)-5-(3-fluoro-5-methylphenyl)pyridin-2-yl)amino)ethyl)amino)-3-oxopropoxy)ethyl)-1H-1,2,3-triazole-4-carboxamido)-3-methylbutanamido)-5-ureidopentanamido)benzyl ((S)-1-(((S)-1-(((3R,4S,5S)-1-((S)-2-((1R,2R)-3-(((1S,2R)-1-hydroxy-1-phenylpropan-2-yl)amino)-1-methoxy-2-methyl-3-oxopropyl)pyrrolidin-1-yl)-3-methoxy-5-methyl-1-oxoheptan-4-yl)(methyl)amino)-3-methyl-1-oxobutan-2-yl)amino)-3-methyl-1-oxobutan-2-yl)(methyl)carbamate (14 mg, 7.3 μmol, 15%); (M+H)⁺=1911.4.

Step E-5, preparation of 1-(S)-1-[N—(S)-1-{[(1S,2R)-4-[(S)-2-{(2R)-2-[N-(1R,2S)-2-hydroxy-1-methyl-2-phenylethylcarbamoyl]-1-methoxypropyl}-1-pyrrolidinyl]-1-[(S)-1-methylpropyl]-2-methoxy-4-oxobutyl]-N-methylcarbamoyl}-2-methylpropylcarbamoyl]-2-methylpropyl 1-methy {p-[(S)-2-[(S)-2-{1-[2-(2-{N-2-[4-(4-amino-1-piperidyl)-3-(6-chloro-1H-1,3-benzimidazol-2-yl)-5-(3-fluoro-5-tolyl)-2-pyridylamino]ethylcarbamoyl}ethoxy)ethyl]-1H-1,2,3-triazol-4-ylcarbonylamino}-3-methylbutyrylamino]-5-ureidovalerylamino]phenyl}methanecarbamate: Into a 8 mL vial was placed a mixture of 4-((S)-2-((S)-2-(1-(2-(3-((2-((4-(4-((tert-butoxycarbonyl)amino)piperidin-1-yl)-3-(6-chloro-1H-benzo[d]imidazol-2-yl)-5-(3-fluoro-5-methylphenyl)pyridin-2-yl)amino)ethyl)amino)-3-oxopropoxy)ethyl)-1H-1,2,3-triazole-4-carboxamido)-3-methylbutanamido)-5-ureidopentanamido)benzyl ((S)-1-(((S)-1-(((3R,4S,5S)-1-((S)-2-((1R,2R)-3-(((1S,2R)-1-hydroxy-1-phenylpropan-2-yl)amino)-1-methoxy-2-methyl-3-oxopropyl)pyrrolidin-1-yl)-3-methoxy-5-methyl-1-oxoheptan-4-yl)(methyl)amino)-3-methyl-1-oxobutan-2-yl)amino)-3-methyl-1-oxobutan-2-yl)(methyl)carbamate (14 mg, 1 Eq. 7.3 μmol), DCM (0.2 mL) and trifluoroacetic acid (0.04 mL), and the reaction mixture was stirred at 0° C. for 30 minutes. The crude product was purified by Prep-HPLC with the following conditions: Column: SunFire prep OBD 19*150 mm 5 um; Mobile Phase A: Water (0.05% trifluoroacetic acid); Mobile Phase B:ACN; Gradient: 25% B to 65% B in 8 min; Flow rate: 20 mL/min; Wave Length: 220 nm. The collected fractions were dried by lyophilization to provide 1-(S)-1-[N—(S)-1-{[(1S,2R)-4-[(S)-2-{(2R)-2-[N-(1R,2S)-2-hydroxy-1-methyl-2-phenylethylcarbamoyl]-1-methoxypropyl}-1-pyrrolidinyl]-1-[(S)-1-methylpropyl]-2-methoxy-4-oxobutyl]-N-methylcarbamoyl}-2-methylpropylcarbamoyl]-2-methylpropyl 1-methyl {p-[(S)-2-[(S)-2-{1-[2-(2-{N-2-[4-(4-amino-1-piperidyl)-3-(6-chloro-1H-1,3-benzimidazol-2-yl)-5-(3-fluoro-5-tolyl)-2-pyridylamino]ethylcarbamoy}ethoxy)ethyl]-1H-1,2,3-triazol-4-ylcarbonylamino}-3-methylbutyrylamino]-5-ureidovalerylamino]phenyl}methanecarbamate (5.4 mg, 2.5 μmol, 34%); (M+H)⁺=1809.9.

The following conjugates were prepared similarly to Example E with appropriate substituting reagents and substrates at different steps and they may require additional functional group modifications via well-known chemistry with appropriate reagents.

| | MS |
|---|---|
| Compound | (M + H)⁺ |
| 1-(S)-1-[N-(S)-1-{[(1S,2R)-4-[(S)-2-{(1R,2R)-2-[N-(1R,2S)-2-hydroxy-1-methyl-2-phenylethylcarbamoyl]-1-methoxypropyl}-1-pyrrolidinyl]- | 2244.2 |
| 1-[(S)-1-methylpropyl]-2-methoxy-4-oxobutyl]-N-methylcarbamoyl}-2-methylpropylcarbamoyl]-2-methylpropyl 1-methyl {p-[(S)-2-[(S)-2-{5- | |
| [(S)-2-[(S)-2-(14-{N-2-[4-(4-amino-1-piperidyl)-3-(6-chloro-1H-1,3-benzimidazol-2-yl)-5-(3-fluoro-5-tolyl)-2-pyridylamino]ethylcarbamoyl}- | |
| 3,6,9,12-tetraoxatetradecylcarbonylamino)-4-carbamoylbutyrylamino]-4-carbamoylbutyrylamino]pentylcarbonylamino}-3- | |
| methylbutyrylamino]-5-ureidovalerylamino]phenyl}methanecarbamate | |
| 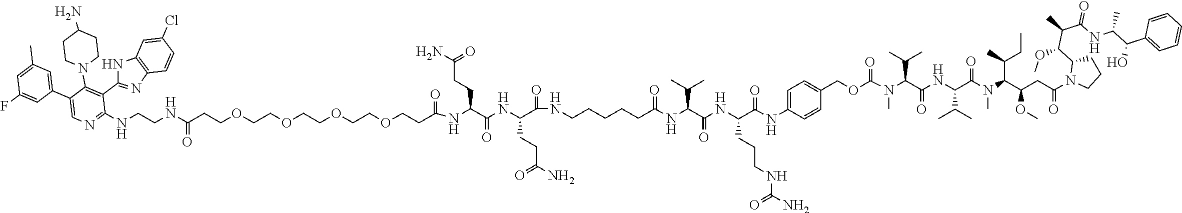 [SMILES: CC[C@H](C)[C@@H]([C@@H](CC(=O)N1CCC[C@H]1[C@H](OC)[C@@H](C)C(=O)N[C@H](C)[C@@H](O)c1ccccc1)OC)N(C)C(=O)[C@@H](NC(=O)[C@H](C(C)C)N(C)C(=O)OCc1ccc(NC(=O)[C@H](CCCNC(N)=O)NC(=O)[C@@H](NC(=O)CCCCCNC(=O)[C@H](CCC(N)=O)NC(=O)[C@H](CCC(N)=O)NC(=O)CCOCCOCCOCCOCCC(=O)NCCNc2ncc(-c3cc(C)cc(F)c3)c(N3CCC(N)CC3)c2-c2nc3ccc(Cl)cc3[nH]2)C(C)C)cc1)C(C)C] | |
| (Compound 143) | |
| {3-[N-(S)-1-[N-(S)-1-[N-p-({[(S)-1-[N-(S)-1-{[(1S,2R)-4-[(S)-2-{(1R,2R)-2-[N-(1R,2S)-2-hydroxy-1-methyl-2-phenylethylcarbamoyl]-1- | 2067.0 |
| methoxypropyl}-1-pyrrolidinyl]-1-[(S)-1-methylpropyl]-2-methoxy-4-oxobutyl]-N-methylcarbamoyl}-2-methylpropylcarbamoyl]-2- | |
| methylpropyl](methyl)(aminocarbonyloxy)}methyl)phenylcarbamoyl]-4-ureidobutylcarbamoyl]-2-methylpropylcarbamoyl]-5-[2-(2-{2-[2-(2- | |
| {N-2-[4-(4-amino-1-piperidyl)-3-(6-chloro-1H-1,3-benzimidazol-2-yl)-5-(3-fluoro-5-tolyl)-2- | |
| pyridylamino]ethylcarbamoyl}ethoxy)ethoxy]ethoxy}ethoxy)ethylamino]benzylamino}acetic acid | |
| 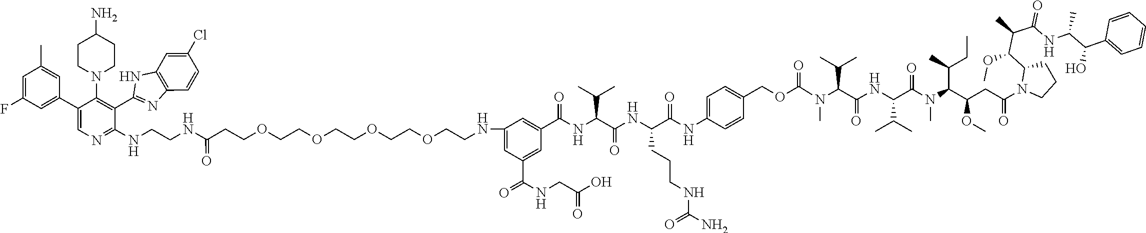 [SMILES: CC[C@H](C)[C@@H]([C@@H](CC(=O)N1CCC[C@H]1[C@H](OC)[C@@H](C)C(=O)N[C@H](C)[C@@H](O)c1ccccc1)OC)N(C)C(=O)[C@@H](NC(=O)[C@H](C(C)C)N(C)C(=O)OCc1ccc(NC(=O)[C@H](CCCNC(N)=O)NC(=O)[C@@H](NC(=O)c2cc(NCCOCCOCCOCCOCCC(=O)NCCNc3ncc(-c4cc(C)cc(F)c4)c(N4CCC(N)CC4)c3-c3nc4ccc(Cl)cc4[nH]3)cc(C(=O)NCC(=O)O)c2)C(C)C)cc1)C(C)C] | |
| (Compound 144) | |
| 3-{3-[N-(S)-1-[N-(S)-1-[N-p-({[(S)-1-[N-(S)-1-{[(1S,2R)-4-[(S)-2-{(1R,2R)-2-[N-(1R,2S)-2-hydroxy-1-methyl-2-phenylethylcarbamoyl]-1- | 1949.0 |
| methoxypropyl}-1-pyrrolidinyl]-1-[(S)-1-methylpropyl]-2-methoxy-4-oxobutyl]-N-methylcarbamoyl}-2-methylpropylcarbamoyl]-2- | |
| methylpropyl](methyl)(aminocarbonyloxy)}methyl)phenylcarbamoyl]-4-ureidobutylcarbamoyl]-2-methylpropylcarbamoyl]-5-[2-(2-{N-2-[4-(4- | |
| amino-1-piperidyl)-3-(6-chloro-1H-1,3-benzimidazol-2-yl)-5-(3-fluoro-5-tolyl)-2- | |
| pyridylamino]ethylcarbamoyl}ethoxy)ethylamino]benzylamino}propionic acid | |
| 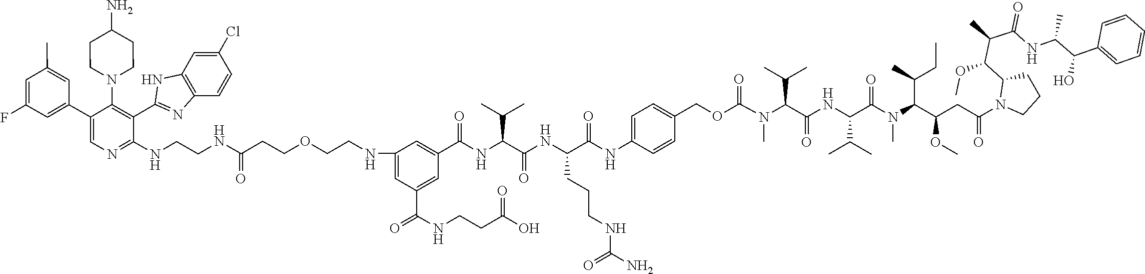 [SMILES: CC[C@H](C)[C@@H]([C@@H](CC(=O)N1CCC[C@H]1[C@H](OC)[C@@H](C)C(=O)N[C@H](C)[C@@H](O)c1ccccc1)OC)N(C)C(=O)[C@@H](NC(=O)[C@H](C(C)C)N(C)C(=O)OCc1ccc(NC(=O)[C@H](CCCNC(N)=O)NC(=O)[C@@H](NC(=O)c2cc(NCCOCCC(=O)NCCNc3ncc(-c4cc(C)cc(F)c4)c(N4CCC(N)CC4)c3-c3nc4ccc(Cl)cc4[nH]3)cc(C(=O)NCCC(=O)O)c2)C(C)C)cc1)C(C)C] | |
| (Compound 145) | |
| 4-{3-[N-(S)-1-[N-(S)-1-[N-p-({[(S)-1-[N-(S)-1-{[(1S,2R)-4-[(S)-2-{(1R,2R)-2-[N-(1R,2S)-2-hydroxy-1-methyl-2-phenylethylcarbamoyl]-1- | 1963.0 |
| methoxypropyl}-1-pyrrolidinyl]-1-[(S)-1-methylpropyl]-2-methoxy-4-oxobutyl]-N-methylcarbamoyl}-2-methylpropylcarbamoyl]-2- | |
| methylpropyl](methyl)(aminocarbonyloxy)}methyl)phenylcarbamoyl]-4-ureidobutylcarbamoyl]-2-methylpropylcarbamoyl]-5-[2-(2-{N-2-[4-(4- | |
| amino-1-piperidyl)-3-(6-chloro-1H-1,3-benzimidazol-2-yl)-5-(3-fluoro-5-tolyl)-2- | |
| pyridylamino]ethylcarbamoyl}ethoxy)ethylamino]benzylamino}butyric acid | |
| 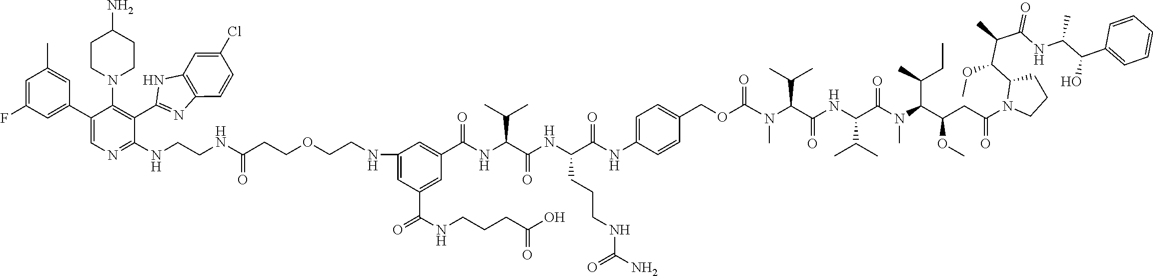 [SMILES: CC[C@H](C)[C@@H]([C@@H](CC(=O)N1CCC[C@H]1[C@H](OC)[C@@H](C)C(=O)N[C@H](C)[C@@H](O)c1ccccc1)OC)N(C)C(=O)[C@@H](NC(=O)[C@H](C(C)C)N(C)C(=O)OCc1ccc(NC(=O)[C@H](CCCNC(N)=O)NC(=O)[C@@H](NC(=O)c2cc(NCCOCCC(=O)NCCNc3ncc(-c4cc(C)cc(F)c4)c(N4CCC(N)CC4)c3-c3nc4ccc(Cl)cc4[nH]3)cc(C(=O)NCCCC(=O)O)c2)C(C)C)cc1)C(C)C] | |
| (Compound 146) | |
| (R)-3-{3-[N-(S)-1-[N-(S)-1-[N-p-({[(S)-1-[N-(S)-1-{[(1S,2R)-4-[(S)-2-{(1R,2R)-2-[N-(1R,2S)-2-hydroxy-1-methyl-2-phenylethylcarbamoyl]- | 1964.0 |
| 1-methoxypropyl}-1-pyrrolidinyl]-1-[(S)-1-methylpropyl]-2-methoxy-4-oxobutyl]-N-methylcarbamoyl}-2-methylpropylcarbamoyl]-2- | |
| methylpropyl](methyl)(aminocarbonyloxy)}methyl)phenylcarbamoyl]-4-ureidobutylcarbamoyl]-2-methylpropylcarbamoyl]-5-[2-(2-{N-2-[4-(4- | |
| amino-1-piperidyl)-3-(6-chloro-1H-1,3-benzimidazol-2-yl)-5-(3-fluoro-5-tolyl)-2- | |
| pyridylamino]ethylcarbamoyl}ethoxy)ethylamino]benzylamino}-2-aminopropionic acid | |
| 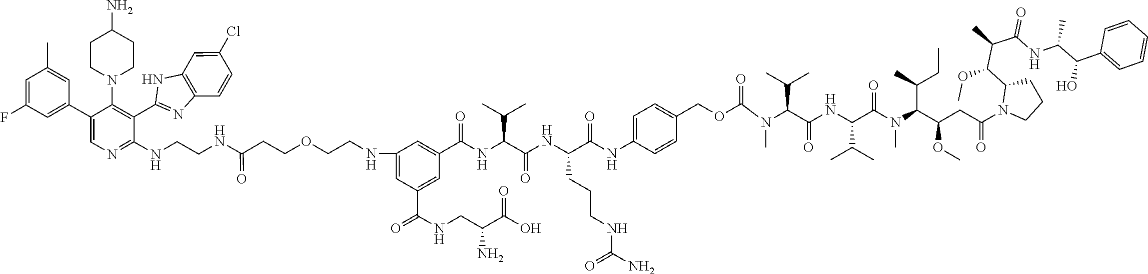 [SMILES: CC[C@H](C)[C@@H]([C@@H](CC(=O)N1CCC[C@H]1[C@H](OC)[C@@H](C)C(=O)N[C@H](C)[C@@H](O)c1ccccc1)OC)N(C)C(=O)[C@@H](NC(=O)[C@H](C(C)C)N(C)C(=O)OCc1ccc(NC(=O)[C@H](CCCNC(N)=O)NC(=O)[C@@H](NC(=O)c2cc(NCCOCCC(=O)NCCNc3ncc(-c4cc(C)cc(F)c4)c(N4CCC(N)CC4)c3-c3nc4ccc(Cl)cc4[nH]3)cc(C(=O)NC[C@@H](N)C(=O)O)c2)C(C)C)cc1)C(C)C] | |
| (Compound 147) | |
| [({3-[N-(S)-1-[N-(S)-1-[N-p-({[(S)-1-[N-(S)-1-{[(1S,2R)-4-[(S)-2-{(1R,2R)-2-[N-(1R,2S)-2-hydroxy-1-methyl-2-phenylethylcarbamoyl]-1- | 1921.0 |
| methoxypropyl}-1-pyrrolidinyl]-1-[(S)-1-methylpropyl]-2-methoxy-4-oxobutyl]-N-methylcarbamoyl}-2-methylpropylcarbamoyl]-2- | |
| methylpropyl](methyl)(aminocarbonyloxy)}methyl)phenylcarbamoyl]-4-ureidobutylcarbamoyl]-2-methylpropylcarbamoyl]-5-[2-(2-{N-[4-(4- | |
| amino-1-piperidyl)-3-(6-chloro-1H-1,3-benzimidazol-2-yl)-5-(3-fluoro-5-tolyl)-2- | |
| pyridylamino]ethylcarbamoyl}ethoxy)ethylamino]phenyl}methyl)amino]acetic acid | |
| 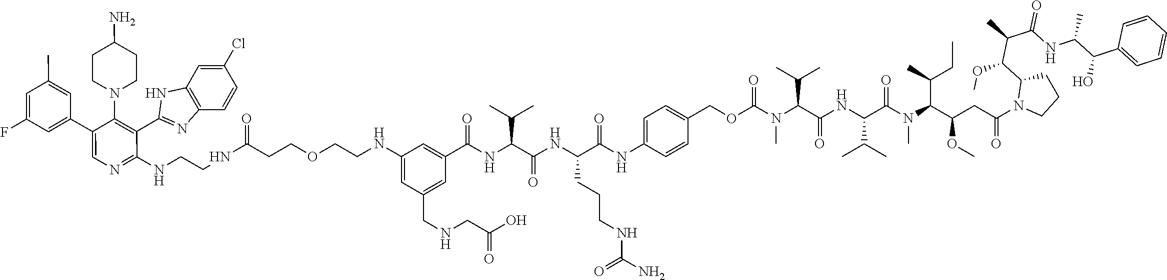 [SMILES: CC[C@H](C)[C@@H]([C@@H](CC(=O)N1CCC[C@H]1[C@H](OC)[C@@H](C)C(=O)N[C@H](C)[C@@H](O)c1ccccc1)OC)N(C)C(=O)[C@@H](NC(=O)[C@H](C(C)C)N(C)C(=O)OCc1ccc(NC(=O)[C@H](CCCNC(N)=O)NC(=O)[C@@H](NC(=O)c2cc(CNCC(=O)O)cc(NCCOCCC(=O)NCCNc3ncc(-c4cc(C)cc(F)c4)c(N4CCC(N)CC4)c3-c3nc4ccc(Cl)cc4[nH]3)c2)C(C)C)cc1)C(C)C] | |
| (Compound 148) | 1963.0 |
| {3-[N-(S)-1-[N-(S)-1-[N-p-({[(S)-1-[N-(S)-1-{[(1S,2R)-4-[(S)-2-{(1R,2R)-2-[N-(1R,2S)-2-hydroxy-1-methyl-2-phenylethylcarbamoyl]-1- | |
| methoxypropyl}-1-pyrrolidinyl]-1-[(S)-1-methylpropyl]-2-methoxy-4-oxobutyl]-N-methylcarbamoyl}-2-methylpropylcarbamoyl]-2- | |
| methylpropyl](methyl)(aminocarbonyloxy)}methyl)phenylcarbamoyl]-4-ureidobutylcarbamoyl]-2-methylpropylcarbamoyl]-5-[3-(2-{N-2-[4-(4- | |
| amino-1-piperidyl)-3-(6-chloro-1H-1,3-benzimidazol-2-yl)-5-(3-fluoro-5-tolyl)-2- | |
| pyridylamino]ethylcarbamoyl}ethoxy)propionylamino]benzylamino}acetic acid | |
| 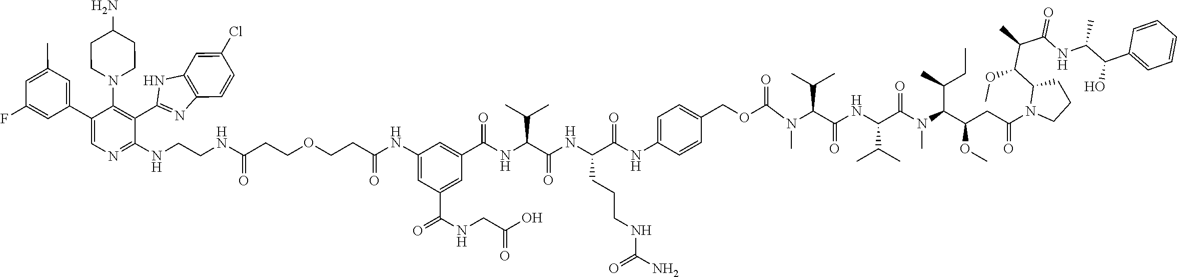 [SMILES: CC[C@H](C)[C@@H]([C@@H](CC(=O)N1CCC[C@H]1[C@H](OC)[C@@H](C)C(=O)N[C@H](C)[C@@H](O)c1ccccc1)OC)N(C)C(=O)[C@@H](NC(=O)[C@H](C(C)C)N(C)C(=O)OCc1ccc(NC(=O)[C@H](CCCNC(N)=O)NC(=O)[C@@H](NC(=O)c2cc(NC(=O)CCOCCC(=O)NCCNc3ncc(-c4cc(C)cc(F)c4)c(N4CCC(N)CC4)c3-c3nc4ccc(Cl)cc4[nH]3)cc(C(=O)NCC(=O)O)c2)C(C)C)cc1)C(C)C] | |
| (Compound 149) | |
| {3-[N-(S)-1-[N-(S)-1-[N-p-({[(S)-1-[N-(S)-1-{[(1S,2R)-4-[(S)-2-{[(1R,2R)-2-[N-(1R,2S)-2-hydroxy-1-methyl-2-phenylethylcarbamoyl]-1- | 2095.0 |
| methoxypropyl}-1-pyrrolidinyl]-1-[(S)-1-methylpropyl]-2-methoxy-4-oxobutyl]-N-methylcarbamoyl}-2-methylpropylcarbamoyl]-2- | |
| methylpropyl](methyl)(aminocarbonyloxy)}methyl)phenylcarbamoyl]-4-ureidobutylcarbamoyl]-2-methylpropylcarbamoyl]-5-(14-{N-2-[4-(4- | |
| amino-1-piperidyl)-3-(6-chloro-1H-1,3-benzimidazol-2-yl)-5-(3-fluoro-5-tolyl)-2-pyridylamino]ethylcarbamoyl}-3,6,9,12- | |
| tetraoxatetradecylcarbonylamino)benzylamino}acetic acid | |
| 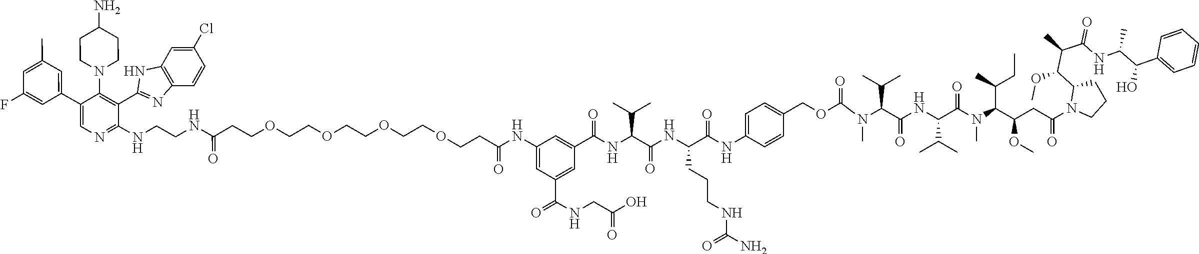 [SMILES: CC[C@H](C)[C@@H]([C@@H](CC(=O)N1CCC[C@H]1[C@H](OC)[C@@H](C)C(=O)N[C@H](C)[C@@H](O)c1ccccc1)OC)N(C)C(=O)[C@@H](NC(=O)[C@H](C(C)C)N(C)C(=O)OCc1ccc(NC(=O)[C@H](CCCNC(N)=O)NC(=O)[C@@H](NC(=O)c2cc(NC(=O)CCOCCOCCOCCOCCC(=O)NCCNc3ncc(-c4cc(C)cc(F)c4)c(N4CCC(N)CC4)c3-c3nc4ccc(Cl)cc4[nH]3)cc(C(=O)NCC(=O)O)c2)C(C)C)cc1)C(C)C] | |
| (Compound 150) | |
| {3-[N-(S)-1-[N-(S)-1-[N-p-({[(S)-1-[N-(S)-1-{[(1S,2R)-4-[(S)-2-{(1R,2R)-2-[N-(1R,2S)-2-hydroxy-1-methyl-2-phenylethylcarbamoyl]-1- | 2034.0 |
| methoxypropyl}-1-pyrrolidinyl]-1-[(S)-1-methylpropyl]-2-methoxy-4-oxobutyl]-N-methylcarbamoyl}-2-methylpropylcarbamoyl]-2- | |
| methylpropyl](methyl)(aminocarbonyloxy)}methyl)phenylcarbamoyl]-4-ureidobutylcarbamoyl]-2-methylpropylcarbamoyl]-5-(3-{2-[({N-2-[4- | |
| (4-amino-1-piperidinyl)-3-(6-chloro-1H-1,3-benzimidazol-2-yl)-5-(3-fluoro-5-tolyl)-2-pyridylamino]ethylcarbamoyl}methyl)-N- | |
| methylcarbamoyl]ethoxy}propionylamino)benzylamino}acetic acid | |
| 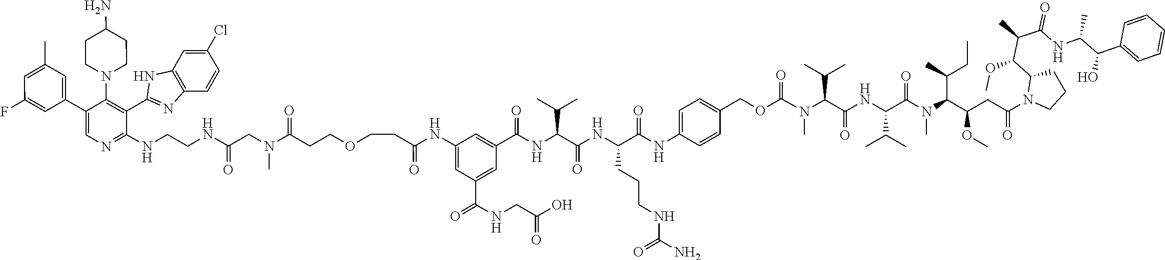 [SMILES: CC[C@H](C)[C@@H]([C@@H](CC(=O)N1CCC[C@H]1[C@H](OC)[C@@H](C)C(=O)N[C@H](C)[C@@H](O)c1ccccc1)OC)N(C)C(=O)[C@@H](NC(=O)[C@H](C(C)C)N(C)C(=O)OCc1ccc(NC(=O)[C@H](CCCNC(N)=O)NC(=O)[C@@H](NC(=O)c2cc(NC(=O)CCOCCC(=O)N(C)CC(=O)NCCNc3ncc(-c4cc(C)cc(F)c4)c(N4CCC(N)CC4)c3-c3nc4ccc(Cl)cc4[nH]3)cc(C(=O)NCC(=O)O)c2)C(C)C)cc1)C(C)C] | |
| (Compound 151) | |
| {3-[N-(S)-1-[N-(S)-1-[N-p-({[(S)-1-[N-(S)-1-{[(1S,2R)-4-[(S)-2-{(1R,2R)-2-[N-(1R,2S)-2-hydroxy-1-methyl-2-phenylethylcarbamoyl]-1- | 2004.0 |
| methoxypropyl}-1-pyrrolidinyl]-1-[(S)-1-methylpropyl]-2-methoxy-4-oxobutyl]-N-methylcarbamoyl}-2-methylpropylcarbamoyl]-2- | |
| methylpropyl](methyl)(aminocarbonyloxy)}methyl)phenylcarbamoyl]-4-ureidobutylcarbamoyl]-2-methylpropylcarbamoyl]-5-{4-[({N-2-[4-(4- | |
| amino-1-piperidyl)-3-(6-chloro-1H-1,3-benzimidazol-2-yl)-5-(3-fluoro-5-tolyl)-2-pyridylamino]ethylcarbamoyl}methyl)-N- | |
| methylcarbamoyl]butyrylamino}benzylamino}acetic acid | |
| 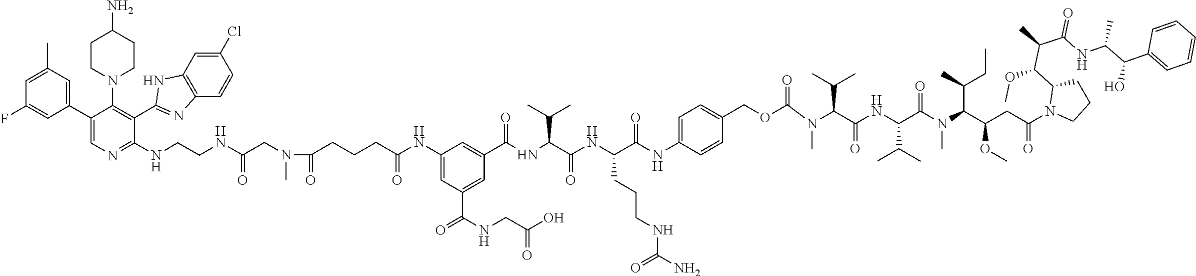 [SMILES: CC[C@H](C)[C@@H]([C@@H](CC(=O)N1CCC[C@H]1[C@H](OC)[C@@H](C)C(=O)N[C@H](C)[C@@H](O)c1ccccc1)OC)N(C)C(=O)[C@@H](NC(=O)[C@H](C(C)C)N(C)C(=O)OCc1ccc(NC(=O)[C@H](CCCNC(N)=O)NC(=O)[C@@H](NC(=O)c2cc(NC(=O)CCCC(=O)N(C)CC(=O)NCCNc3ncc(-c4cc(C)cc(F)c4)c(N4CCC(N)CC4)c3-c3nc4ccc(Cl)cc4[nH]3)cc(C(=O)NCC(=O)O)c2)C(C)C)cc1)C(C)C] | |
| (Compound 152) | |
| {3-[N-(S)-1-[N-(S)-1-[N-p-({[(S)-1-[N-(S)-1-{[(1S,2R)-4-[(S)-2-{(1R,2R)-2-[N-(1R,2S)-2-hydroxy-1-methyl-2-phenylethylcarbamoyl]-1- | 1947.9 |
| methoxypropyl}-1-pyrrolidinyl]-1-[(S)-1-methylpropyl]-2-methoxy-4-oxobutyl]-N-methylcarbamoyl}-2-methylpropylcarbamoyl]-2- | |
| methylpropyl](methyl)(aminocarbonyloxy)}methyl)phenylcarbamoyl]ethylcarbamoyl]-2-methylpropylcarbamoyl]-5-(3-{2-[({N-2-[4-(4-amino- | |
| 1-piperidyl)-3-(6-chloro-1H-1,3-benzimidazol-2-yl)-5-(3-fluoro-5-tolyl)-2-pyridylamino]ethylcarbamoyl}methyl)-N- | |
| methylcarbamoyl]ethoxy}propionylamino)benzylamino}acetic acid | |
| 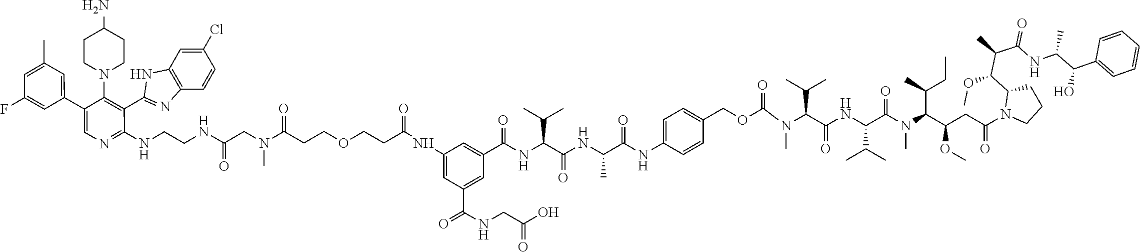 [SMILES: CC[C@H](C)[C@@H]([C@@H](CC(=O)N1CCC[C@H]1[C@H](OC)[C@@H](C)C(=O)N[C@H](C)[C@@H](O)c1ccccc1)OC)N(C)C(=O)[C@@H](NC(=O)[C@H](C(C)C)N(C)C(=O)OCc1ccc(NC(=O)[C@H](C)NC(=O)[C@@H](NC(=O)c2cc(NC(=O)CCOCCC(=O)N(C)CC(=O)NCCNc3ncc(-c4cc(C)cc(F)c4)c(N4CCC(N)CC4)c3-c3nc4ccc(Cl)cc4[nH]3)cc(C(=O)NCC(=O)O)c2)C(C)C)cc1)C(C)C] | |
| (Compound 153) | |
| {3-[N-(S)-1-[N-(S)-1-[N-p-({[(S)-1-[N-(S)-1-{[(1S,2R)-4-[(S)-2-{(1R,2R)-2-[N-(1R,2S)-2-hydroxy-1-methyl-2-phenylethylcarbamoyl]-1- | 1933.0 |
| methoxypropyl}-1-pyrrolidinyl]-1-[(S)-1-methylpropyl]-2-methoxy-4-oxobutyl]-N-methylcarbamoyl}-2-methylpropylcarbamoyl]-2- | |
| methylpropyl](methyl)(aminocarbonyloxy)}methyl)phenylcarbamoyl]-4-ureidobutylcarbamoyl]-2-methylpropylcarbamoyl]-5-(5-{N-2-[4-(4- | |
| amino-1-piperidyl)-3-(6-chloro-1H-1,3-benzimidazol-2-yl)-5-(3-fluoro-5-tolyl)-2- | |
| pyridylamino]ethylcarbamoyl}pentylamino)benzylamino}acetic acid | |
| 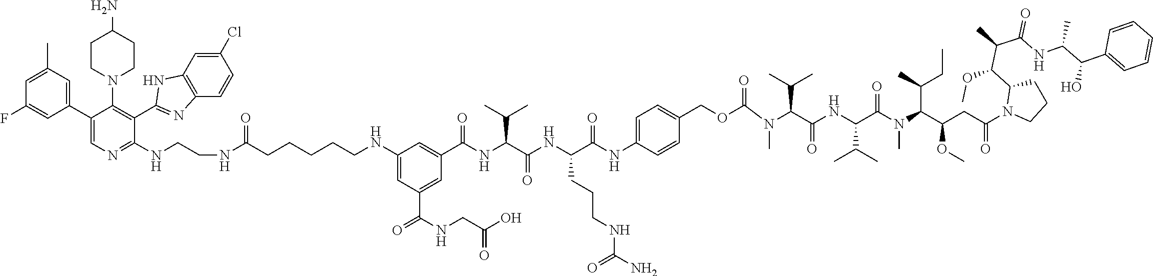 [SMILES: CC[C@H](C)[C@@H]([C@@H](CC(=O)N1CCC[C@H]1[C@H](OC)[C@@H](C)C(=O)N[C@H](C)[C@@H](O)c1ccccc1)OC)N(C)C(=O)[C@@H](NC(=O)[C@H](C(C)C)N(C)C(=O)OCc1ccc(NC(=O)[C@H](CCCNC(N)=O)NC(=O)[C@@H](NC(=O)c2cc(NCCCCCC(=O)NCCNc3ncc(-c4cc(C)cc(F)c4)c(N4CCC(N)CC4)c3-c3nc4ccc(Cl)cc4[nH]3)cc(C(=O)NCC(=O)O)c2)C(C)C)cc1)C(C)C] | |
| (Compound 154) | |
| {3-[N-(S)-1-[N-(S)-1-[N-p-({[(S)-1-[N-(S)-1-{[(1S,2R)-4-[(S)-2-{(1R,2R)-2-[N-(1R,2S)-2-hydroxy-1-methyl-2-phenylethylcarbamoyl]-1- | 2058.0 |
| methoxypropyl}-1-pyrrolidinyl]-1-[(S)-1-methylpropyl]-2-methoxy-4-oxobutyl]-N-methylcarbamoyl}-2-methylpropylcarbamoyl]-2- | |
| methylpropyl](methyl)(aminocarbonyloxy)}methyl)phenylcarbamoyl]-4-ureidobutylcarbamoyl]-2-methylpropylcarbamoyl]-5-(3-{2-[4-(2-{N- | |
| 2-[4-(4-amino-1-piperidyl)-3-(6-chloro-1H-1,3-benzimidazol-2-yl)-5-(3-fluoro-5-tolyl)-2-pyridylamino]ethylcarbamoyl}ethyl)-1H-1,2,3- | |
| triazol-1-yl]ethoxy}propionylamino)benzylamino}acetic acid | |
| 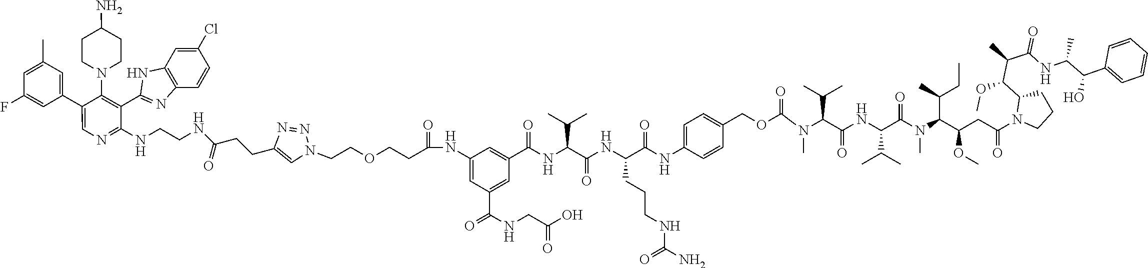 [SMILES: CC[C@H](C)[C@@H]([C@@H](CC(=O)N1CCC[C@H]1[C@H](OC)[C@@H](C)C(=O)N[C@H](C)[C@@H](O)c1ccccc1)OC)N(C)C(=O)[C@@H](NC(=O)[C@H](C(C)C)N(C)C(=O)OCc1ccc(NC(=O)[C@H](CCCNC(N)=O)NC(=O)[C@@H](NC(=O)c2cc(NC(=O)CCOCCn3cc(CCC(=O)NCCNc4ncc(-c5cc(C)cc(F)c5)c(N5CCC(N)CC5)c4-c4nc5ccc(Cl)cc5[nH]4)nn3)cc(C(=O)NCC(=O)O)c2)C(C)C)cc1)C(C)C] | |
| (Compound 155) | |
| {3-[N-(S)-1-[N-(S)-1-[N-p-({[(S)-1-[N-(S)-1-{[(1S,2R)-4-[(S)-2-{(1R,2R)-2-[N-(1R,2S)-2-hydroxy-1-methyl-2-phenylethylcarbamoyl]-1- | 2049.0 |
| methoxypropyl}-1-pyrrolidinyl]-1-[(S)-1-methylpropyl]-2-methoxy-4-oxobutyl]-N-methylcarbamoyl}-2-methylpropylcarbamoyl]-2- | |
| methylpropyl](methyl)(aminocarbonyloxy)}methyl)phenylcarbamoyl]-4-ureidobutylcarbamoyl]-2-methylpropylcarbamoyl]-5-[(R)-3-[N-2-(2- | |
| {N-2-[4-(4-amino-1-piperidyl)-3-(6-chloro-1H-1,3-benzimidazol-2-yl)-5-(3-fluoro-5-tolyl)-2- | |
| pyridylamino]ethylcarbamoyl}ethoxy)ethylcarbamoyl]-3-aminopropionylamino]benzylamino}acetic acid | |
| 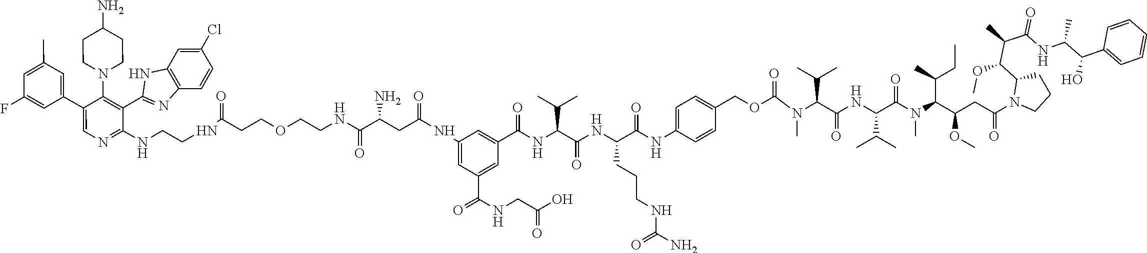 [SMILES: CC[C@H](C)[C@@H]([C@@H](CC(=O)N1CCC[C@H]1[C@H](OC)[C@@H](C)C(=O)N[C@H](C)[C@@H](O)c1ccccc1)OC)N(C)C(=O)[C@@H](NC(=O)[C@H](C(C)C)N(C)C(=O)OCc1ccc(NC(=O)[C@H](CCCNC(N)=O)NC(=O)[C@@H](NC(=O)c2cc(NC(=O)C[C@@H](N)C(=O)NCCOCCC(=O)NCCNc3ncc(-c4cc(C)cc(F)c4)c(N4CCC(N)CC4)c3-c3nc4ccc(Cl)cc4[nH]3)cc(C(=O)NCC(=O)O)c2)C(C)C)cc1)C(C)C] | |
| (Compound 156) | |
| {3-[N-(S)-1-[N-(S)-1-[N-p-({[(S)-1-[N-(S)-1-{[(1S,2R)-4-[(S)-2-{(1R,2R)-2-[N-(1R,2S)-2-hydroxy-1-methyl-2-phenylethylcarbamoyl]-1- | 1891.0 |
| methoxypropyl}-1-pyrrolidinyl]-1-[(1S)-1-methylpropyl]-2-methoxy-4-oxobutyl]-N-methylcarbamoyl}-2-methylpropylcarbamoyl]-2- | |
| methylpropyl](methyl)(aminocarbonyloxy)}methyl)phenylcarbamoyl]-4-ureidobutylcarbamoyl]-2-methylpropylcarbamoyl]-5-(2-{N-2-[4-(4- | |
| amino-1-piperidyl)-3-(6-chloro-1H-1,3-benzimidazol-2-yl)-5-(3-fluoro-5-tolyl)-2- | |
| pyridylamino]ethylcarbamoyl}ethylamino)benzylamino}acetic acid | |
| 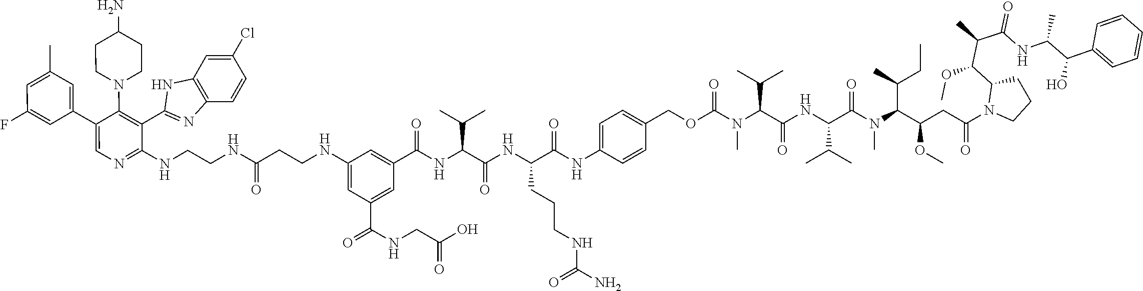 [SMILES: CC[C@H](C)[C@@H]([C@@H](CC(=O)N1CCC[C@H]1[C@H](OC)[C@@H](C)C(=O)N[C@H](C)[C@@H](O)c1ccccc1)OC)N(C)C(=O)[C@@H](NC(=O)[C@H](C(C)C)N(C)C(=O)OCc1ccc(NC(=O)[C@H](CCCNC(N)=O)NC(=O)[C@@H](NC(=O)c2cc(NCCC(=O)NCCNc3ncc(-c4cc(C)cc(F)c4)c(N4CCC(N)CC4)c3-c3nc4ccc(Cl)cc4[nH]3)cc(C(=O)NCC(=O)O)c2)C(C)C)cc1)C(C)C] | |
| (Compound 157) | |
| {3-[N-(S)-1-[N-(S)-1-[N-p-({[(S)-1-[N-(S)-1-{[(1S,2R)-4-[(S)-2-{(1R,2R)-2-[N-(1R,2S)-2-hydroxy-1-methyl-2-phenylethylcarbamoyl]-1- | 1949.0 |
| methoxypropyl}-1-pyrrolidinyl]-1-[(S)-1-methylpropyl]-2-methoxy-4-oxobutyl]-N-methylcarbamoyl}-2-methylpropylcarbamoyl]-2- | |
| methylpropyl](methyl)(aminocarbonyloxy)}methyl)phenylcarbamoyl]-4-ureidobutylcarbamoyl]-2-methylpropylcarbamoyl]-5-{[2-(2-{N-2-[4- | |
| (4-amino-1-piperidyl)-3-(6-chloro-1H-1,3-benzimidazol-2-yl)-5-(3-fluoro-5-tolyl)-2-pyridylamino]ethylcarbamoyl}ethoxy)ethyl]-N- | |
| methylamino}benzylamino}acetic acid | |
| 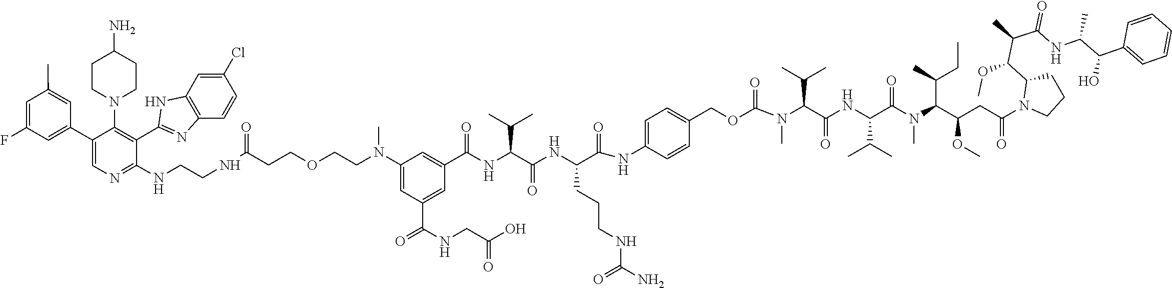 [SMILES: CC[C@H](C)[C@@H]([C@@H](CC(=O)N1CCC[C@H]1[C@H](OC)[C@@H](C)C(=O)N[C@H](C)[C@@H](O)c1ccccc1)OC)N(C)C(=O)[C@@H](NC(=O)[C@H](C(C)C)N(C)C(=O)OCc1ccc(NC(=O)[C@H](CCCNC(N)=O)NC(=O)[C@@H](NC(=O)c2cc(C(=O)NCC(=O)O)cc(N(C)CCOCCC(=O)NCCNc3ncc(-c4cc(C)cc(F)c4)c(N4CCC(N)CC4)c3-c3nc4ccc(Cl)cc4[nH]3)c2)C(C)C)cc1)C(C)C] | |
| (Compound 158) | |
| {3-[N-(S)-1-[N-(S)-1-[N-p-({[(S)-1-[N-(S)-1-{[(1S,2R)-4-[(S)-2-{(1R,2R)-2-[N-(1R,2S)-2-hydroxy-1-methyl-2-phenylethylcarbamoyl]-1- | 2081.1 |
| methoxypropyl}-1-pyrrolidinyl]-1-[(S)-1-methylpropyl]-2-methoxy-4-oxobutyl]-N-methylcarbamoyl}-2-methylpropylcarbamoyl]-2- | |
| methylpropyl](methyl)(aminocarbonyloxy)}methyl)phenylcarbamoyl]-4-ureidobutylcarbamoyl]-2-methylpropylcarbamoyl]-5-(15-{N-[4-(4- | |
| amino-1-piperidyl)-3-(6-chloro-1H-1,3-benzimidazol-2-yl)-5-(3-fluoro-5-tolyl)-2-pyridylamino]ethylcarbamoyl}-1-methyl-4,7,10,13-tetraoxa- | |
| 1-azapentadecyl)benzylamino}acetic acid | |
| 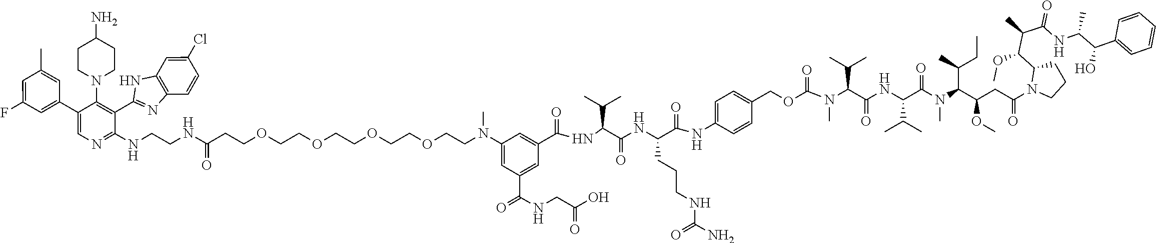 [SMILES: CC[C@H](C)[C@@H]([C@@H](CC(=O)N1CCC[C@H]1[C@H](OC)[C@@H](C)C(=O)N[C@H](C)[C@@H](O)c1ccccc1)OC)N(C)C(=O)[C@@H](NC(=O)[C@H](C(C)C)N(C)C(=O)OCc1ccc(NC(=O)[C@H](CCCNC(N)=O)NC(=O)[C@@H](NC(=O)c2cc(C(=O)NCC(=O)O)cc(N(C)CCOCCOCCOCCOCCC(=O)NCCNc3ncc(-c4cc(C)cc(F)c4)c(N4CCC(N)CC4)c3-c3nc4ccc(Cl)cc4[nH]3)c2)C(C)C)cc1)C(C)C] | |
| (Compound 159) | |
| {3-[N-(S)-1-[N-(S)-1-[N-p-({[(S)-1-[N-(S)-1-{[(1S,2R)-4-[(S)-2-{(1R,2R)-2-[N-(1R,2S)-2-hydroxy-1-methyl-2-phenylethylcarbamoyl]-1- | 1936.0 |
| methoxypropyl}-1-pyrrolidinyl]-1-[(S)-1-methylpropyl]-2-methoxy-4-oxobutyl]-N-methylcarbamoyl}-2-methylpropylcarbamoyl]-2- | |
| methylpropyl](methyl)(aminocarbonyloxy)}methyl)phenylcarbamoyl]-4-ureidobutylcarbamoyl]-2-methylpropylcarbamoyl]-5-[2-(2-{N-2-[4-(4- | |
| amino-1-piperidyl)-3-(6-chloro-1H-1,3-benzimidazol-2-yl)-5-(3-fluoro-5-tolyl)-2- | |
| pyridylamino]ethylcarbamoyl}ethoxy)ethoxy]benzylamino}acetic acid | |
| 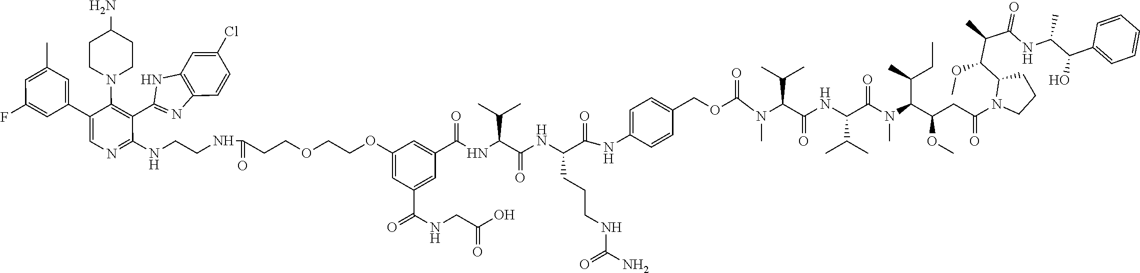 [SMILES: CC[C@H](C)[C@@H]([C@@H](CC(=O)N1CCC[C@H]1[C@H](OC)[C@@H](C)C(=O)N[C@H](C)[C@@H](O)c1ccccc1)OC)N(C)C(=O)[C@@H](NC(=O)[C@H](C(C)C)N(C)C(=O)OCc1ccc(NC(=O)[C@H](CCCNC(N)=O)NC(=O)[C@@H](NC(=O)c2cc(OCCOCCC(=O)NCCNc3ncc(-c4cc(C)cc(F)c4)c(N4CCC(N)CC4)c3-c3nc4ccc(Cl)cc4[nH]3)cc(C(=O)NCC(=O)O)c2)C(C)C)cc1)C(C)C] | |
| (Compound 160) | |
| {3-[N-(S)-1-[N-(S)-1-[N-p-({[(S)-1-[N-(S)-1-{[(1S,2R)-4-[(S)-2-{(1R,2R)-2-[N-(1R,2S)-2-hydroxy-1-methyl-2-phenylethylcarbamoyl]-1- | 2068.0 |
| methoxypropyl}-1-pyrrolidinyl]-1-[(S)-1-methylpropyl]-2-methoxy-4-oxobutyl]-N-methylcarbamoyl}-2-methylpropylcarbamoyl]-2- | |
| methylpropyl](methyl)(aminocarbonyloxy)}methyl)phenylcarbamoyl]-4-ureidobutylcarbamoyl]-2-methylpropylcarbamoyl]-5-[2-(2-{2-[2-(2- | |
| {N-2-[4-(4-amino-1-piperidyl)-3-(6-chloro-1H-1,3-benzimidazol-2-yl)-5-(3-fluoro-5-tolyl)-2- | |
| pyridylamino]ethylcarbamoyl}ethoxy)ethoxy]ethoxy}ethoxy)ethoxy]benzylamino}acetic acid | |
| 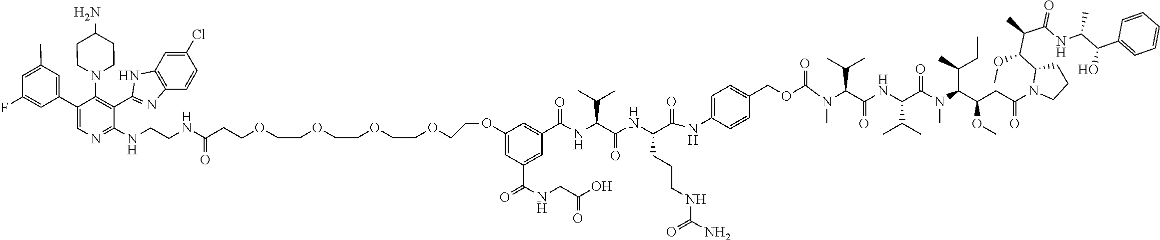 [SMILES: CC[C@H](C)[C@@H]([C@@H](CC(=O)N1CCC[C@H]1[C@H](OC)[C@@H](C)C(=O)N[C@H](C)[C@@H](O)c1ccccc1)OC)N(C)C(=O)[C@@H](NC(=O)[C@H](C(C)C)N(C)C(=O)OCc1ccc(NC(=O)[C@H](CCCNC(N)=O)NC(=O)[C@@H](NC(=O)c2cc(OCCOCCOCCOCCOCCC(=O)NCCNc3ncc(-c4cc(C)cc(F)c4)c(N4CCC(N)CC4)c3-c3nc4ccc(Cl)cc4[nH]3)cc(C(=O)NCC(=O)O)c2)C(C)C)cc1)C(C)C] | |
| (Compound 161) | |
| {3-[N-(S)-1-[N-(S)-1-[N-p-({[(S)-1-[N-(S)-1-{[(1S,2R)-4-[(S)-2-{(1R,2R)-2-[N-(1R,2S)-2-hydroxy-1-methyl-2-phenylethylcarbamoyl]-1- | 2006.0 |
| methoxypropyl}-1-pyrrolidinyl]-1-[(S)-1-methylpropyl]-2-methoxy-4-oxobutyl]-N-methylcarbamoyl}-2-methylpropylcarbamoyl]-2- | |
| methylpropyl](methyl)(aminocarbonyloxy)}methyl)phenylcarbamoyl]-4-ureidobutylcarbamoyl]-2-methylpropylcarbamoyl]-5-(2-{2-[({N-2-[4- | |
| (4-amino-1-piperidyl)-3-(6-chloro-1H-1,3-benzimidazol-2-yl)-5-(3-fluoro-5-tolyl)-2-pyridylamino]ethylcarbamoyl}methyl)-N- | |
| methylcarbamoyl]ethoxy}ethylamino)benzylamino}acetic acid | |
| 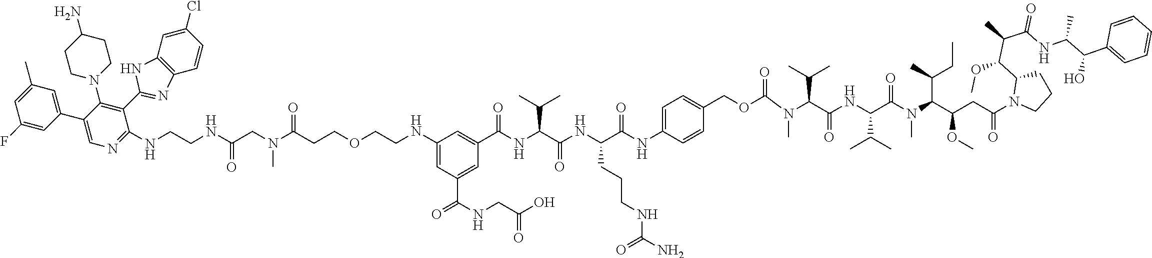 [SMILES: CC[C@H](C)[C@@H]([C@@H](CC(=O)N1CCC[C@H]1[C@H](OC)[C@@H](C)C(=O)N[C@H](C)[C@@H](O)c1ccccc1)OC)N(C)C(=O)[C@@H](NC(=O)[C@H](C(C)C)N(C)C(=O)OCc1ccc(NC(=O)[C@H](CCCNC(N)=O)NC(=O)[C@@H](NC(=O)c2cc(NCCOCCC(=O)N(C)CC(=O)NCCNc3ncc(-c4cc(C)cc(F)c4)c(N4CCC(N)CC4)c3-c3nc4ccc(Cl)cc4[nH]3)cc(C(=O)NCC(=O)O)c2)C(C)C)cc1)C(C)C] | |
| (Compound 162) | |
| [3-(N-2-{2-[(R)-3-[N-(S)-1-[N-(S)-1-[N-p-({[(S)-1-[N-(S)-1-{[(1S,2R)-4-[(S)-2-{(1R,2R)-2-[N-(1R,2S)-2-hydroxy-1-methyl-2- | 2136.1 |
| phenylethylcarbamoyl]-1-methoxypropyl}-1-pyrrolidinyl]-1-[(S)-1-methylpropyl]-2-methoxy-4-oxobutyl]-N-methylcarbamoyl}-2- | |
| methylpropylcarbamoyl]-2-methylpropyl](methyl)(aminocarbonyloxy)}methyl)phenylcarbamoyl]-4-ureidobutylcarbamoyl]-2- | |
| methylpropylcarbamoyl]-2-aminopropionylamino]ethoxy}ethylcarbamoyl)-5-[2-(2-{N-2-[4-(4-amino-1-piperidyl)-3-(6-chloro-1H-1,3- | |
| benzimidazol-2-yl)-5-(3-fluoro-5-tolyl)-2-pyridylamino]ethylcarbamoyl}ethoxy)ethylamino]benzylamino]acetic acid | |
| 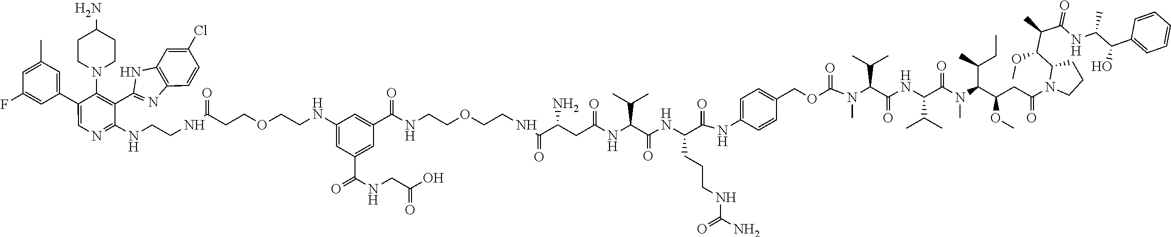 [SMILES: CC[C@H](C)[C@@H]([C@@H](CC(=O)N1CCC[C@H]1[C@H](OC)[C@@H](C)C(=O)N[C@H](C)[C@@H](O)c1ccccc1)OC)N(C)C(=O)[C@@H](NC(=O)[C@H](C(C)C)N(C)C(=O)OCc1ccc(NC(=O)[C@H](CCCNC(N)=O)NC(=O)[C@@H](NC(=O)C[C@@H](N)C(=O)NCCOCCNC(=O)c2cc(NCCOCCC(=O)NCCNc3ncc(-c4cc(C)cc(F)c4)c(N4CCC(N)CC4)c3-c3nc4ccc(Cl)cc4[nH]3)cc(C(=O)NCC(=O)O)c2)C(C)C)cc1)C(C)C] | |
| (Compound 163) | |
| [3-(N-2-{2-[(R)-3-[N-(S)-1-[N-(S)-1-[N-p-({[(S)-1-[N-(S)-1-{[(1S,2R)-4-[(S)-2-{(1R,2R)-2-[N-(1R,2S)-2-hydroxy-1-methyl-2- | 2150.1 |
| phenylethylcarbamooyl]-1-methoxypropyl}-1-pyrrolidinyl]-1-[(S)-1-methylpropyl]-2-methoxy-4-oxobutyl]-N-methylcarbamoyl}-2- | |
| methylpropylcarbamoyl]-2-methylpropyl](methyl)(aminocarbonyloxy)}methyl)phenylcarbamoyl]-4-ureidobutylcarbamoyl]-2- | |
| methylpropylcarbamoyl]-2-aminopropionylamino]ethoxy}ethylcarbamoyl)-5-[2-(2-{N-2-[4-(4-amino-1-piperidyl)-3-(5-fluoro-7-methoxy-1H- | |
| 1,3-benzimidazol-2-yl)-5-(3-fluoro-5-tolyl)-2-pyridylamino]ethylcarbamoyl}ethoxy)ethylamino]benzylamino]acetic acid | |
| 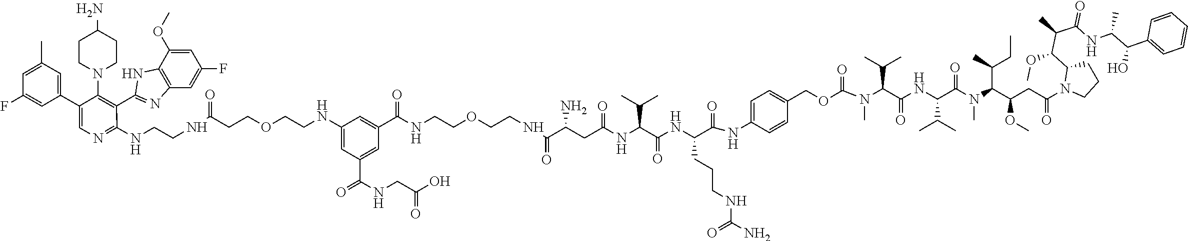 [SMILES: CC[C@H](C)[C@@H]([C@@H](CC(=O)N1CCC[C@H]1[C@H](OC)[C@@H](C)C(=O)N[C@H](C)[C@@H](O)c1ccccc1)OC)N(C)C(=O)[C@@H](NC(=O)[C@H](C(C)C)N(C)C(=O)OCc1ccc(NC(=O)[C@H](CCCNC(N)=O)NC(=O)[C@@H](NC(=O)C[C@@H](N)C(=O)NCCOCCNC(=O)c2cc(NCCOCCC(=O)NCCNc3ncc(-c4cc(C)cc(F)c4)c(N4CCC(N)CC4)c3-c3nc4cc(F)cc(OC)c4[nH]3)cc(C(=O)NCC(=O)O)c2)C(C)C)cc1)C(C)C] | |
| (Compound 164) | |
| {3-[N-(S)-1-[N-(S)-1-[N-p-({[(S)-1-[N-(S)-1-{[(1S,2R)-4-[(S)-2-{(1R,2R)-2-[N-(1R,2S)-2-hydroxy-1-methyl-2-phenylethylcarbamoyl]-1- | 1919.6 |
| methoxypropyl}-1-pyrrolidinyl]-1-[(S)-1-methylpropyl]-2-methoxy-4-oxobutyl]-N-methylcarbamoyl}-2-methylpropylcarbamoyl]-2- | |
| methylpropyl](methyl)(aminocarbonyloxy)}methyl)phenylcarbamoyl]-4-ureidobutylcarbamoyl]-2-methylpropylcarbamoyl]-5-[2-(2-{N-2-[4-(4- | |
| amino-1-piperidyl)-3-(6-fluoro-1H-1,3-benzimidazol-2-yl)-5-(3-fluoro-5-tolyl)-2- | |
| pyridylamino]ethylcarbamoyl}ethoxy)ethylamino]benzylamino}acetic acid | |
| 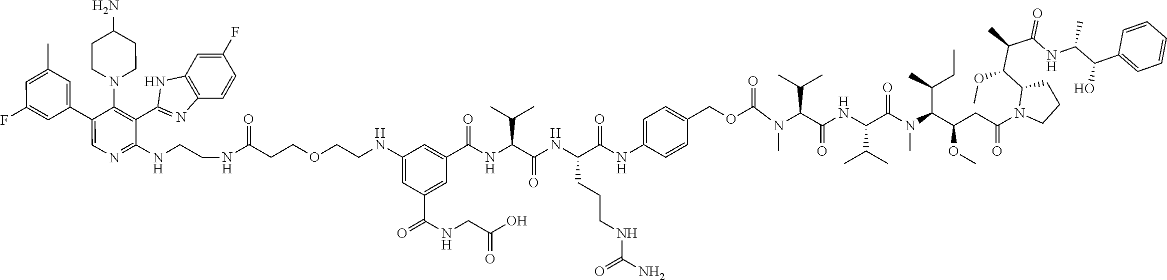 [SMILES: CC[C@H](C)[C@@H]([C@@H](CC(=O)N1CCC[C@H]1[C@H](OC)[C@@H](C)C(=O)N[C@H](C)[C@@H](O)c1ccccc1)OC)N(C)C(=O)[C@@H](NC(=O)[C@H](C(C)C)N(C)C(=O)OCc1ccc(NC(=O)[C@H](CCCNC(N)=O)NC(=O)[C@@H](NC(=O)c2cc(NCCOCCC(=O)NCCNc3ncc(-c4cc(C)cc(F)c4)c(N4CCC(N)CC4)c3-c3nc4ccc(F)cc4[nH]3)cc(C(=O)NCC(=O)O)c2)C(C)C)cc1)C(C)C] | |
| (Compound 165) | |
| {3-[N-(S)-1-[N-(S)-1-[N-p-({[(S)-1-[N-(S)-1-{[(1S,2R)-4-[(S)-2-{(1R,2R)-2-[N-(1R,2S)-2-hydroxy-1-methyl-2-phenylethylcarbamoyl]-1- | 1949.0 |
| methoxypropyl}-1-pyrrolidinyl]-1-[(S)-1-methylpropyl]-2-methoxy-4-oxobutyl]-N-methylcarbamoyl}-2-methylpropylcarbamoyl]-2- | |
| methylpropyl](methyl)(aminocarbonyloxy)}methyl)phenylcarbamoyl]-4-ureidobutylcarbamoyl]-2-methylpropylcarbamoyl]-5-[2-(2-{N-2-[4-(4- | |
| amino-1-piperidyl)-3-(5-fluoro-7-methoxy-1H-1,3-benzimidazol-2-yl)-5-(3-fluoro-5-tolyl)-2- | |
| pyridylamino]ethylcarbamoyl}ethoxy)ethylamino]benzylamino}acetic acid | |
| 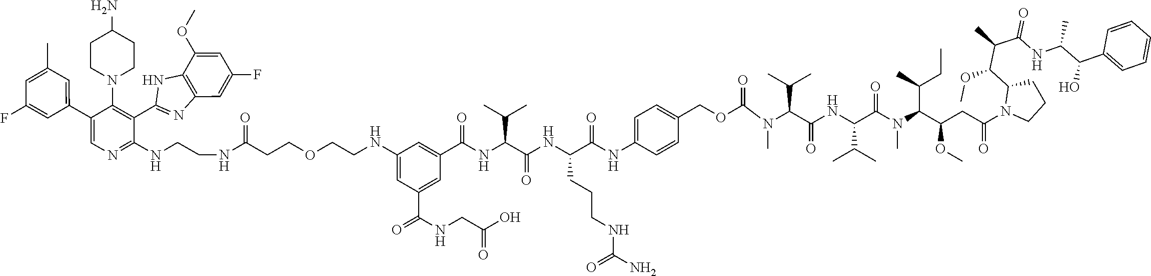 [SMILES: CC[C@H](C)[C@@H]([C@@H](CC(=O)N1CCC[C@H]1[C@H](OC)[C@@H](C)C(=O)N[C@H](C)[C@@H](O)c1ccccc1)OC)N(C)C(=O)[C@@H](NC(=O)[C@H](C(C)C)N(C)C(=O)OCc1ccc(NC(=O)[C@H](CCCNC(N)=O)NC(=O)[C@@H](NC(=O)c2cc(NCCOCCC(=O)NCCNc3ncc(-c4cc(C)cc(F)c4)c(N4CCC(N)CC4)c3-c3nc4cc(F)cc(OC)c4[nH]3)cc(C(=O)NCC(=O)O)c2)C(C)C)cc1)C(C)C] | |
| (Compound 166) | |
| {3-[N-(S)-1-[N-(S)-1-[N-p-({[(S)-1-[N-(S)-1-{[(1S,2R)-4-[(S)-2-{(1R,2R)-2-[N-(1R,2S)-2-hydroxy-1-methyl-2-phenylethylcarbamoyl]-1- | 1917.0 |
| methoxypropyl}-1-pyrrolidinyl]-1-[(S)-1-methylpropyl]-2-methoxy-4-oxobutyl]-N-methylcarbamoyl}-2-methylpropylcarbamoyl]-2- | |
| methylpropyl](methyl)(aminocarbonyloxy)}methyl)phenylcarbamoyl]-4-ureidobutylcarbamoyl]-2-methylpropylcarbamoyl]-5-(5-{N-2-[4-(4- | |
| amino-1-piperidyl)-3-(6-fluoro-1H-1,3-benzimidazol-2-yl)-5-(3-fluoro-5-tolyl)-2- | |
| pyridylamino]ethylcarbamoyl}pentylamino)benzylamino}acetic acid | |
| 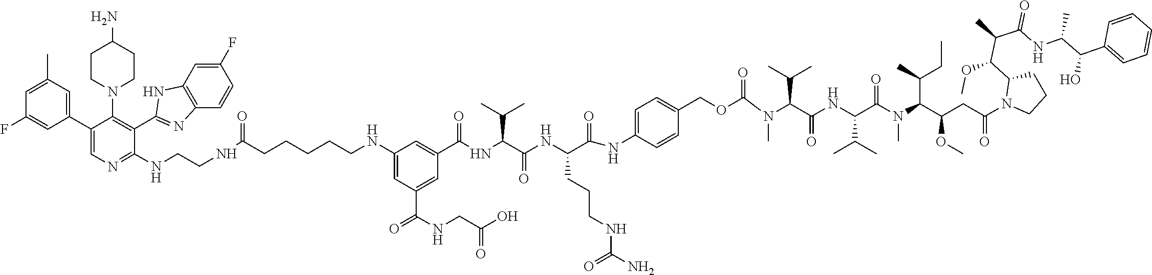 [SMILES: CC[C@H](C)[C@@H]([C@@H](CC(=O)N1CCC[C@H]1[C@H](OC)[C@@H](C)C(=O)N[C@H](C)[C@@H](O)c1ccccc1)OC)N(C)C(=O)[C@@H](NC(=O)[C@H](C(C)C)N(C)C(=O)OCc1ccc(NC(=O)[C@H](CCCNC(N)=O)NC(=O)[C@@H](NC(=O)c2cc(NCCCCCC(=O)NCCNc3ncc(-c4cc(C)cc(F)c4)c(N4CCC(N)CC4)c3-c3nc4ccc(F)cc4[nH]3)cc(C(=O)NCC(=O)O)c2)C(C)C)cc1)C(C)C] | |
| (Compound 167) | |
| {3-[N-(S)-1-[N-(S)-1-[N-p-({[(S)-1-[N-(S)-1-{[(1S,2R)-4-[(S)-2-{(1R,2R)-2-[N-(1R,2S)-2-hydroxy-1-methyl-2-phenylethylcarbamoyl]-1- | 1988.1 |
| methoxypropyl}-1-pyrrolidinyl]-1-[(S)-1-methylpropyl]-2-methoxy-4-oxobutyl]-N-methylcarbamoyl}-2-methylpropylcarbamoyl]-2- | |
| methylpropyl](methyl)(aminocarbonyloxy)}methyl)phenylcarbamoyl]-4-ureidobutylcarbamoyl]-2-methylpropylcarbamoyl]-5-{5-[({N-2-[4-(4- | |
| amino-1-piperidyl)-3-(6-fluoro-1H-1,3-benzimidazol-2-yl)-5-(3-fluoro-5-tolyl)-2-pyridylamino]ethylcarbamoyl}methyl)-N- | |
| methylcarbamoyl]pentylamino}benzylamino}acetic acid | |
| 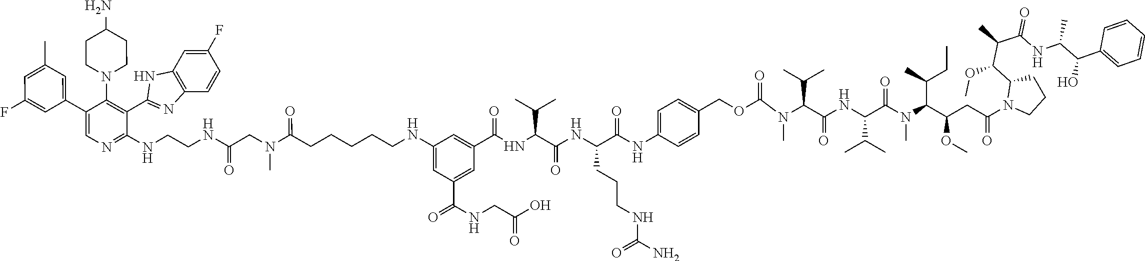 [SMILES: CC[C@H](C)[C@@H]([C@@H](CC(=O)N1CCC[C@H]1[C@H](OC)[C@@H](C)C(=O)N[C@H](C)[C@@H](O)c1ccccc1)OC)N(C)C(=O)[C@@H](NC(=O)[C@H](C(C)C)N(C)C(=O)OCc1ccc(NC(=O)[C@H](CCCNC(N)=O)NC(=O)[C@@H](NC(=O)c2cc(NCCCCCC(=O)N(C)CC(=O)NCCNc3ncc(-c4cc(C)cc(F)c4)c(N4CCC(N)CC4)c3-c3nc4ccc(F)cc4[nH]3)cc(C(=O)NCC(=O)O)c2)C(C)C)cc1)C(C)C] | |
| (Compound 168) | |
| {3-[N-(S)-1-[N-(S)-1-[N-p-({[(S)-1-[N-(S)-1-{[(1S,2R)-4-[(S)-2-{(1R,2R)-2-[N-(1R,2S)-2-hydroxy-1-methyl-2-phenylethylcarbamoyl]-1- | 1988.0 |
| methoxypropyl}-1-pyrrolidinyl]-1-[(S)-1-methylpropyl]-2-methoxy-4-oxobutyl]-N-methylcarbamoyl}-2-methylpropylcarbamoyl]-2- | |
| methylpropyl](methyl)(aminocarbonyloxy)}methyl)phenylcarbamoyl]-4-ureidobutylcarbamoyl]-2-methylpropylcarbamoyl]-5-{4-[({N-2-[4-(4- | |
| amino-1-piperidyl)-3-(6-fluoro-1H-1,3-benzimidazol-2-yl)-5-(3-fluoro-5-tolyl)-2-pyridylamino]ethylcarbamoyl}methyl)-N- | |
| methylcarbamoyl]butyrylamino}benzylamino}acetic acid | |
| 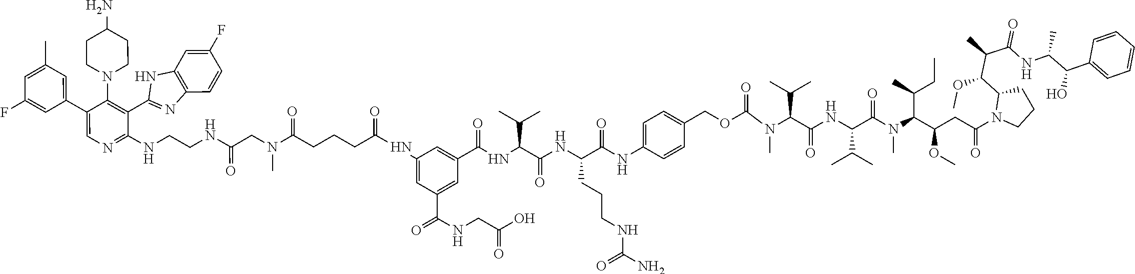 [SMILES: CC[C@H](C)[C@@H]([C@@H](CC(=O)N1CCC[C@H]1[C@H](OC)[C@@H](C)C(=O)N[C@H](C)[C@@H](O)c1ccccc1)OC)N(C)C(=O)[C@@H](NC(=O)[C@H](C(C)C)N(C)C(=O)OCc1ccc(NC(=O)[C@H](CCCNC(N)=O)NC(=O)[C@@H](NC(=O)c2cc(NC(=O)CCCC(=O)N(C)CC(=O)NCCNc3ncc(-c4cc(C)cc(F)c4)c(N4CCC(N)CC4)c3-c3nc4ccc(F)cc4[nH]3)cc(C(=O)NCC(=O)O)c2)C(C)C)cc1)C(C)C] | |
| (Compound 169) | |
| (R)-3-{3-[N-(S)-1-[N-(S)-1-[N-p-({[(S)-1-[N-(S)-1-{[(1S,2R)-4-[(S)-2-{(1R,2R)-2-[N-(1R,2S)-2-hydroxy-1-methyl-2-phenylethylcarbamoyl]- | 1962.0 |
| 1-methoxypropyl}-1-pyrrolidinyl]-1-[(S)-1-methylpropyl]-2-methoxy-4-oxobutyl]-N-methylcarbamoyl}-2-methylpropylcarbamoyl]-2- | |
| methylpropyl](methyl)(aminocarbonyloxy)}methyl)phenylcarbamoyl]-4-ureidobutylcarbamoyl]-2-methylpropylcarbamoyl]-5-(4-{N-2-[4-(4- | |
| amino-1-piperidyl)-3-(6-chloro-1H-1,3-benzimidazol-2-yl)-5-(3-fluoro-5-tolyl)-2-pyridylamino]ethylcarbamoyl}butyrylamino)benzylamino}- | |
| 2-aminopropionic acid | |
| 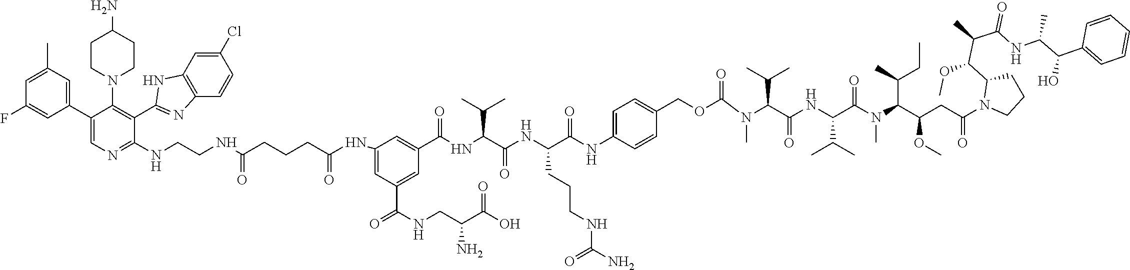 [SMILES: CC[C@H](C)[C@@H]([C@@H](CC(=O)N1CCC[C@H]1[C@H](OC)[C@@H](C)C(=O)N[C@H](C)[C@@H](O)c1ccccc1)OC)N(C)C(=O)[C@@H](NC(=O)[C@H](C(C)C)N(C)C(=O)OCc1ccc(NC(=O)[C@H](CCCNC(N)=O)NC(=O)[C@@H](NC(=O)c2cc(NC(=O)CCCC(=O)NCCNc3ncc(-c4cc(C)cc(F)c4)c(N4CCC(N)CC4)c3-c3nc4ccc(Cl)cc4[nH]3)cc(C(=O)NC[C@@H](N)C(=O)O)c2)C(C)C)cc1)C(C)C] | |
| (Compound 170) | |
| (R)-3-{3-[N-(S)-1-[N-(S)-1-[N-p-({[(S)-1-[N-(S)-1-{[(1S,2R)-4-[(S)-2-{(1R,2R)-2-[N-(1R,2S)-2-hydroxy-1-methyl-2-phenylethylcarbamoyl]- | 2033.0 |
| 1-methoxypropyl}-1-pyrrolidinyl]-1-[(S)-1-methylpropyl]-2-methoxy-4-oxobutyl]-N-methylcarbamoyl}-2-methylpropylcarbamoyl]-2- | |
| methylpropyl](methyl)(aminocarbonyloxy)}methyl)phenylcarbamoyl]-4-ureidobutylcarbamoyl]-2-methylpropylcarbamoyl]-5-{4-[({N-2-[4-(4- | |
| amino-1-piperidyl)-3-(6-chloro-1H-1,3-benzimidazol-2-yl)-5-(3-fluoro-5-tolyl)-2-pyridylamino]ethylcarbamoyl}methyl)-N- | |
| methylcarbamoyl]butyrylamino}benzylamino}-2-aminopropanoic acid | |
| 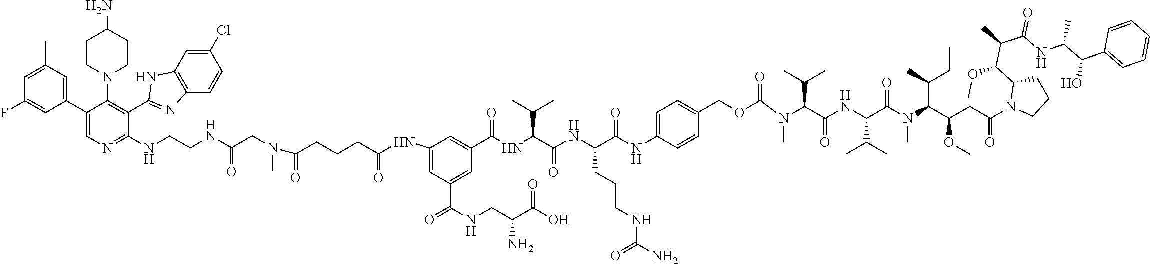 [SMILES: CC[C@H](C)[C@@H]([C@@H](CC(=O)N1CCC[C@H]1[C@H](OC)[C@@H](C)C(=O)N[C@H](C)[C@@H](O)c1ccccc1)OC)N(C)C(=O)[C@@H](NC(=O)[C@H](C(C)C)N(C)C(=O)OCc1ccc(NC(=O)[C@H](CCCNC(N)=O)NC(=O)[C@@H](NC(=O)c2cc(NC(=O)CCCC(=O)N(C)CC(=O)NCCNc3ncc(-c4cc(C)cc(F)c4)c(N4CCC(N)CC4)c3-c3nc4ccc(Cl)cc4[nH]3)cc(C(=O)NC[C@@H](N)C(=O)O)c2)C(C)C)cc1)C(C)C] | |
| (Compound 171) | |
| (R)-3-{3-[N-(S)-1-[N-(S)-1-[N-p-({[(S)-1-{[(1S,2R)-4-[(S)-2-{(1R,2R)-2-[N-(1R,2S)-2-hydroxy-1-methyl-2-phenylethylcarbamoyl]- | 2005.0 |
| 1-methoxypropyl}-1-pyrrolidinyl]-1-[(S)-1-methylpropyl]-2-methoxy-4-oxobutyl]-N-methylcarbamoyl}-2-methylpropylcarbamoyl]-2- | |
| methylpropyl](methyl)(aminocarbonyloxy)}methyl)phenylcarbamoyl]-4-ureidobutylcarbamoyl]-2-methylpropylcarbamoyl]-5-{3-[({N-2-[4-(4- | |
| amino-1-piperidyl)-3-(6-chloro-1H-1,3-benzimidazol-2-yl)-5-(3-fluoro-5-tolyl)-2-pyridylamino]ethylcarbamoyl}methyl)-N- | |
| methylcarbamoyl]propylamino}benzylamino}-2-aminopropionic acid | |
| 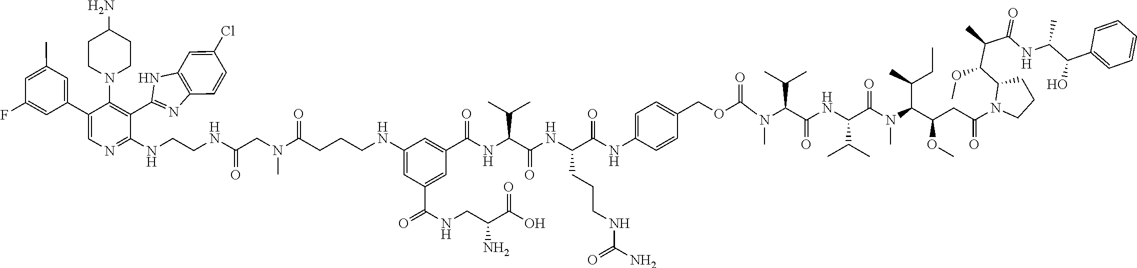 [SMILES: CC[C@H](C)[C@@H]([C@@H](CC(=O)N1CCC[C@H]1[C@H](OC)[C@@H](C)C(=O)N[C@H](C)[C@@H](O)c1ccccc1)OC)N(C)C(=O)[C@@H](NC(=O)[C@H](C(C)C)N(C)C(=O)OCc1ccc(NC(=O)[C@H](CCCNC(N)=O)NC(=O)[C@@H](NC(=O)c2cc(NCCCC(=O)N(C)CC(=O)NCCNc3ncc(-c4cc(C)cc(F)c4)c(N4CCC(N)CC4)c3-c3nc4ccc(Cl)cc4[nH]3)cc(C(=O)NC[C@@H](N)C(=O)O)c2)C(C)C)cc1)C(C)C] | |
| (Compound 172) | |
| {4-[N-(S)-1-[N-(S)-1-[N-p-({[(S)-1-[N-(S)-1-{[(1S,2R)-4-[(S)-2-{(1R,2R)-2-[N-(1R,2S)-2-hydroxy-1-methyl-2-phenylethylcarbamoyl]-1- | 1968.0 |
| methoxypropyl}-1-pyrrolidinyl]-1-[(S)-1-methylpropyl]-2-methoxy-4-oxobutyl]-N-methylcarbamoyl}-2-methylpropylcarbamoyl]-2- | |
| methylpropyl](methyl)(aminocarbonyloxy)}methyl)phenylcarbamoyl]-4-ureidobutylcarbamoyl]-2-methylpropylcarbamoyl]-4-{4-[({N-2-[4-(4- | |
| amino-1-piperidyl)-3-(6-chloro-1H-1,3-benzimidazol-2-yl)-5-(3-fluoro-5-tolyl)-2-pyridylamino]ethylcarbamoyl}methyl)-N- | |
| methylcarbamoyl]butyrylamino}-1-piperidyl}acetic acid | |
| 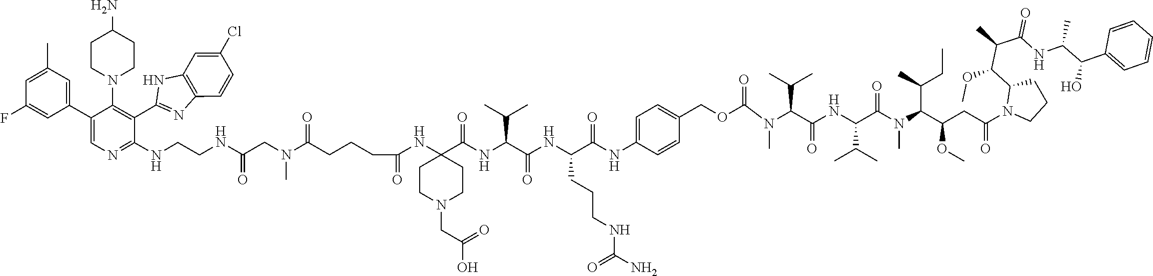 [SMILES: CC[C@H](C)[C@@H]([C@@H](CC(=O)N1CCC[C@H]1[C@H](OC)[C@@H](C)C(=O)N[C@H](C)[C@@H](O)c1ccccc1)OC)N(C)C(=O)[C@@H](NC(=O)[C@H](C(C)C)N(C)C(=O)OCc1ccc(NC(=O)[C@H](CCCNC(N)=O)NC(=O)[C@@H](NC(=O)C2(NC(=O)CCCC(=O)N(C)CC(=O)NCCNc3ncc(-c4cc(C)cc(F)c4)c(N4CCC(N)CC4)c3-c3nc4ccc(Cl)cc4[nH]3)CCN(CC(=O)O)CC2)C(C)C)cc1)C(C)C] | |
| (Compound 173) | |
| {3-[3-(2-{N-2-[4-(4-amino-1-piperidyl)-3-(6-chloro-1H-1,3-benzimidazol-2-yl)-5-(3-fluoro-5-tolyl)-2- | 1899.0 |
| pyridylamino]ethylcarbamoyl}ethoxy)propionylamino]-3-[N-(S)-2-methyl-1-[N-(S)-1-[N-p-({methyl[(S)-2-methyl-1-[N-(S)-1-{[(1S,2R)-2- | |
| methoxy-4-[(S)-2-[(1R,2R)-1-methoxy-2-[N-(1R,2S)-2-hydroxy-1-methyl-2-phenylethylcarbamoyl]propyl]-1-pyrrolidinyl]-1-[(S)-1- | |
| methylpropyl]-4-oxobutyl]-N-methylcarbamoyl}-2-methylpropylcarbamoyl]propyl](aminocarbonyloxy)}methyl)phenylcarbamoyl]-4- | |
| ureidobutylcarbamoyl]propylcarbamoyl]-1-azetidinyl}acetic acid | |
| 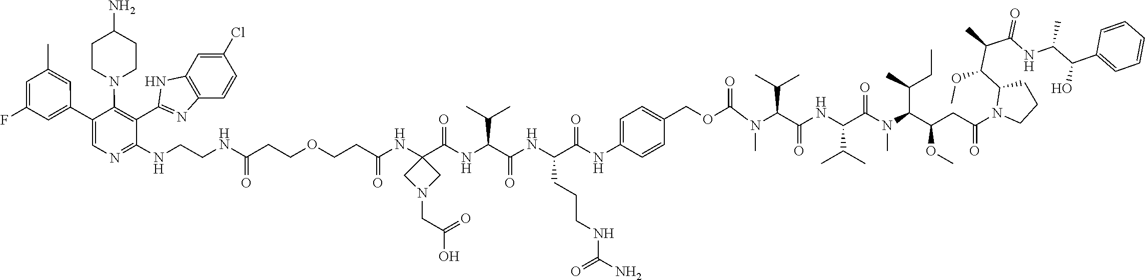 [SMILES: CC[C@H](C)[C@@H]([C@@H](CC(=O)N1CCC[C@H]1[C@H](OC)[C@@H](C)C(=O)N[C@H](C)[C@@H](O)c1ccccc1)OC)N(C)C(=O)[C@@H](NC(=O)[C@H](C(C)C)N(C)C(=O)OCc1ccc(NC(=O)[C@H](CCCNC(N)=O)NC(=O)[C@@H](NC(=O)C2(NC(=O)CCOCCC(=O)NCCNc3ncc(-c4cc(C)cc(F)c4)c(N4CCC(N)CC4)c3-c3nc4ccc(Cl)cc4[nH]3)CN(CC(=O)O)C2)C(C)C)cc1)C(C)C] | |
| (Compound 174) | |
| 1-methyl 1-(S)-2-methyl-1-[N-(S)-1-{[(1S,2R)-2-methoxy-4-[(S)-2-[(1R,2R)-1-methoxy-2-[N-(1R,2S)-2-hydroxy-1-methyl-2- | 2076.1 |
| phenylethylcarbamoyl]propyl]-1-pyrrolidinyl]-1-[(S)-1-methylpropyl]-4-oxobutyl]-N-methylcarbamoyl}-2-methylpropylcarbamoyl]propyl {p- | |
| [(S)-2-[(S)-3-methyl-2-(5-{[(N-2-aminoethylcarbamoyl)methyl]carbamoyl}-3-(3-{2-[N-methyl({N-2-[4-(4-amino-1-piperidyl)-3-(6-chloro-1H- | |
| 1,3-benzimidazol-2-yl)-5-(3-fluoro-5-tolyl)-2- | |
| pyridylamino]ethylcarbamoyl}methyl)carbamoyl]ethoxy}propionylamino)benzylamino)butyrylamino]-5- | |
| ureidovalerylamino]phenyl}methanecarbamate | |
| 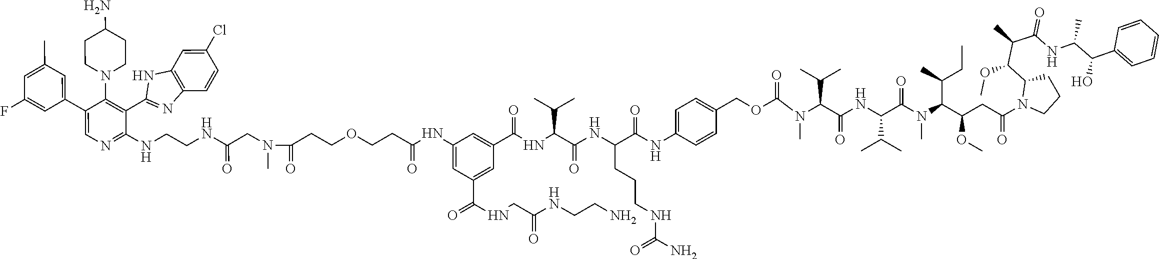 [SMILES: CC[C@H](C)[C@@H]([C@@H](CC(=O)N1CCC[C@H]1[C@H](OC)[C@@H](C)C(=O)N[C@H](C)[C@@H](O)c1ccccc1)OC)N(C)C(=O)[C@@H](NC(=O)[C@H](C(C)C)N(C)C(=O)OCc1ccc(NC(=O)C(CCCNC(N)=O)NC(=O)[C@@H](NC(=O)c2cc(NC(=O)CCOCCC(=O)N(C)CC(=O)NCCNc3ncc(-c4cc(C)cc(F)c4)c(N4CCC(N)CC4)c3-c3nc4ccc(Cl)cc4[nH]3)cc(C(=O)NCC(=O)NCCN)c2)C(C)C)cc1)C(C)C] | |
| (Compound 175) | |
| 1-(S)-1-[N-(S)-1-{[(1S,2R)-4-[(S)-2-{(2R)-2-[N-(1R,2S)-2-hydroxy-1-methyl-2-phenylethylcarbamoyl]-1-methoxypropyl}-1-pyrrolidinyl]-1- | 1896.0 |
| [(S)-1-methylpropyl]-2-methoxy-4-oxobutyl]-N-methylcarbamoyl}-2-methylpropylcarbamoyl]-2-methylpropyl 1-methyl {p-[(S)-2-[(S)-2-[(S)- | |
| 2-[3-(2-{N-2-[4-(4-amino-1-piperidyl)-3-(6-chloro-1H-1,3-benzimidazol-2-yl)-5-(3-fluoro-5-tolyl)-2- | |
| pyridylamino]ethylcarbamoyl}ethoxy)propionylamino]-4-(1H-tetrazol-5-yl)butyrylamino]-3-methylbutyrylamino]-5- | |
| ureidovalerylamino]phenyl}methanecarbamate | |
| 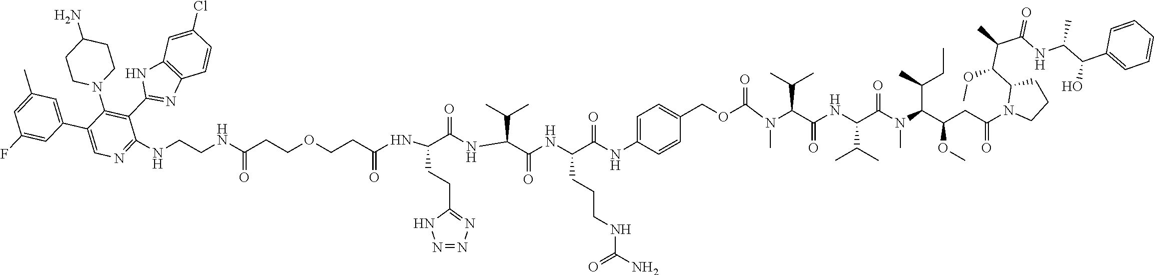 [SMILES: CC[C@H](C)[C@@H]([C@@H](CC(=O)N1CCC[C@H]1[C@H](OC)[C@@H](C)C(=O)N[C@H](C)[C@@H](O)c1ccccc1)OC)N(C)C(=O)[C@@H](NC(=O)[C@H](C(C)C)N(C)C(=O)OCc1ccc(NC(=O)[C@H](CCCNC(N)=O)NC(=O)[C@@H](NC(=O)[C@H](CCc2nnn[nH]2)NC(=O)CCOCCC(=O)NCCNc2ncc(-c3cc(C)cc(F)c3)c(N3CCC(N)CC3)c2-c2nc3ccc(Cl)cc3[nH]2)C(C)C)cc1)C(C)C] | |
| (Compound 176) | |
| 3-[N-(S)-1-[N-(S)-1-[N-(p-({[(S)-1-[N-(S)-1-{[(1S,2R)-4-[(S)-2-{(1R,2R)-2-[N-(1R,2S)-2-hydroxy-1-methyl-2-phenylethylcarbamoyl]-1- | 1966.0 |
| methoxypropyl}-1-pyrrolidinyl]-1-[(S)-1-methylpropyl]-2-methoxy-4-oxobutyl]-N-methylcarbamoyl}-2-methylpropylcarbamoyl]-2- | |
| methylpropyl](methyl)(aminocarbonyloxy)}methyl)phenylcarbamoyl]-4-ureidobutylcarbamoyl]-2-methylpropylcarbamoyl]-5-(2-{2-[2-(2-{N- | |
| 2-[4-(4-amino-1-piperidyl)-3-(6-chloro-1H-1,3-benzimidazol-2-yl)-5-(3-fluoro-5-tolyl)-2- | |
| pyridylamino]ethylcarbamoyl}ethoxy)ethoxy]ethoxy}ethylamino)benzoic acid | |
| 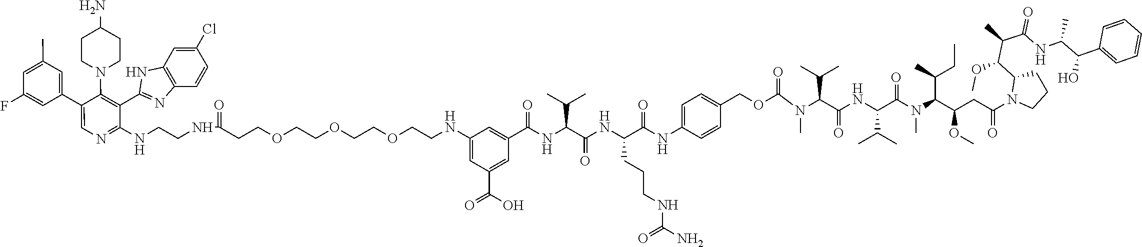 [SMILES: CC[C@H](C)[C@@H]([C@@H](CC(=O)N1CCC[C@H]1[C@H](OC)[C@@H](C)C(=O)N[C@H](C)[C@@H](O)c1ccccc1)OC)N(C)C(=O)[C@@H](NC(=O)[C@H](C(C)C)N(C)C(=O)OCc1ccc(NC(=O)[C@H](CCCNC(N)=O)NC(=O)[C@@H](NC(=O)c2cc(NCCOCCOCCOCCC(=O)NCCNc3ncc(-c4cc(C)cc(F)c4)c(N4CCC(N)CC4)c3-c3nc4ccc(Cl)cc4[nH]3)cc(C(=O)O)c2)C(C)C)cc1)C(C)C] | |
| (Compound 177) | |
| methyl 3-[N-(S)-1-[N-(S)-1-[N-p-({[(S)-1-[N-(S)-1-{[(1S,2R)-4-[(S)-2-{(1R,2R)-2-[N-(1R,2S)-2-hydroxy-1-methyl-2-phenylethylcarbamoyl]- | 1980.0 |
| 1-methoxypropyl}-1-pyrrolidinyl]-1-[(S)-1-methylpropyl]-2-methoxy-4-oxobutyl]-N-methylcarbamoyl}-2-methylpropylcarbamoyl]-2- | |
| methylpropyl](methyl)(aminocarbonyloxy)}methyl)phenylcarbamoyl]-4-ureidobutylcarbamoyl]-2-methylpropylcarbamoyl]-5-(2-{2-[2-(2-{N- | |
| 2-[4-(4-amino-1-piperidyl)-3-(6-chloro-1H-1,3-benzimidazol-2-yl)-5-(3-fluoro-5-tolyl)-2- | |
| pyridylamino]ethylcarbamoyl}ethoxy)ethoxy]ethoxy}ethylamino)benzoate | |
| 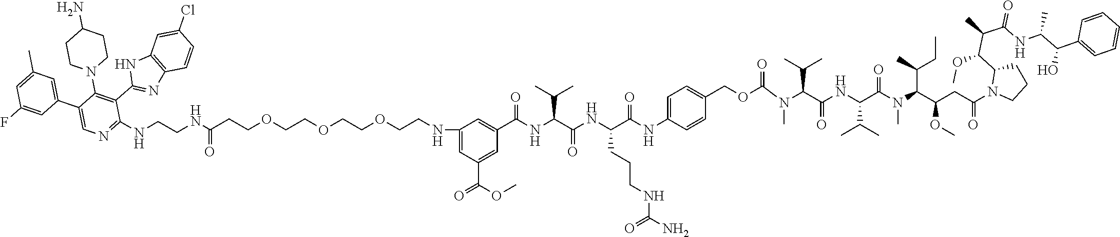 [SMILES: CC[C@H](C)[C@@H]([C@@H](CC(=O)N1CCC[C@H]1[C@H](OC)[C@@H](C)C(=O)N[C@H](C)[C@@H](O)c1ccccc1)OC)N(C)C(=O)[C@@H](NC(=O)[C@H](C(C)C)N(C)C(=O)OCc1ccc(NC(=O)[C@H](CCCNC(N)=O)NC(=O)[C@@H](NC(=O)c2cc(NCCOCCOCCOCCC(=O)NCCNc3ncc(-c4cc(C)cc(F)c4)c(N4CCC(N)CC4)c3-c3nc4ccc(Cl)cc4[nH]3)cc(C(=O)OC)c2)C(C)C)cc1)C(C)C] | |
| (Compound 178) | |
| 1-(S)-1-[N-(S)-1-{[(1S,2R)-4-[(S)-2-{(1R,2R)-2-[N-(1R,2S)-2-hydroxy-1-methyl-2-phenylethylcarbamoyl]-1-methoxypropyl}-1-pyrrolidinyl]- | 2008.1 |
| 1-[(S)-1-methylpropyl]-2-methoxy-4-oxobutyl]-N-methylcarbamoyl}-2-methylpropylcarbamoyl]-2-methylpropyl 1-methyl {p-[(S)-2-[(S)-2-[3- | |
| (N-2-aminoethylcarbamoyl)-5-(2-{2-[2-(2-{N-2-[4-(4-amino-1-piperidyl)-3-(6-chloro-1H-1,3-benzimidazol-2-yl)-5-(3-fluoro-5-tolyl)-2- | |
| pyridylamino]ethylcarbamoyl}ethoxy)ethoxy]ethoxy}ethylamino)benzylamino]-3-methylbutyrylamino]-5- | |
| ureidovalerylamino]phenyl}methanecarbamate | |
| 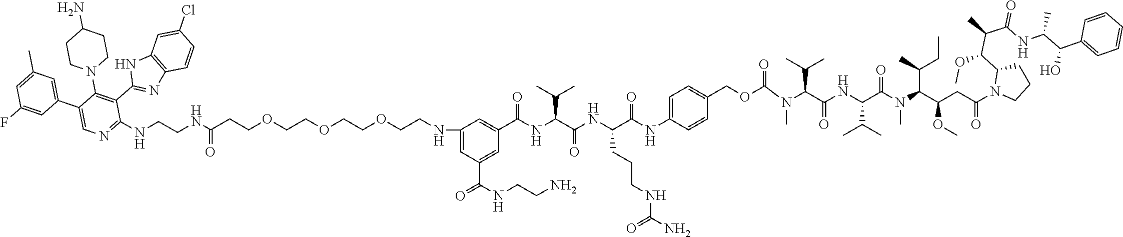 [SMILES: CC[C@H](C)[C@@H]([C@@H](CC(=O)N1CCC[C@H]1[C@H](OC)[C@@H](C)C(=O)N[C@H](C)[C@@H](O)c1ccccc1)OC)N(C)C(=O)[C@@H](NC(=O)[C@H](C(C)C)N(C)C(=O)OCc1ccc(NC(=O)[C@H](CCCNC(N)=O)NC(=O)[C@@H](NC(=O)c2cc(NCCOCCOCCOCCC(=O)NCCNc3ncc(-c4cc(C)cc(F)c4)c(N4CCC(N)CC4)c3-c3nc4ccc(Cl)cc4[nH]3)cc(C(=O)NCCN)c2)C(C)C)cc1)C(C)C] | |
| (Compound 179) | |
| 1-(S)-1-[N-(S)-1-{[(1S,2R)-4-[(S)-2-{(1R,2R)-2-[N-(1R,2S)-2-hydroxy-1-methyl-2-phenylethylcarbamoyl]-1-methoxypropyl}-1-pyrrolidinyl]- | 1968.0 |
| 1-[(S)-1-methylpropyl]-2-methoxy-4-oxobutyl]-N-methylcarbamoyl}-2-methylpropylcarbamoyl]-2-methylpropyl 1-methyl {p-[(S)-2-[(S)-2-{3- | |
| [2-(2-{2-[2-(5-{N-2-[4-(4-amino-1-piperidyl)-3-(6-chloro-1H-1,3-benzimidazol-2-yl)-5-(3-fluoro-5-tolyl)-2-pyridylamino]ethylcarbamoyl}-2- | |
| pyrimidinylamino)ethoxy]ethoxy}ethoxy)ethoxy]propionylamino}-3-methylbutyrylamino]-5-ureidovalerylamino]phenyl}methanecarbamate | |
| 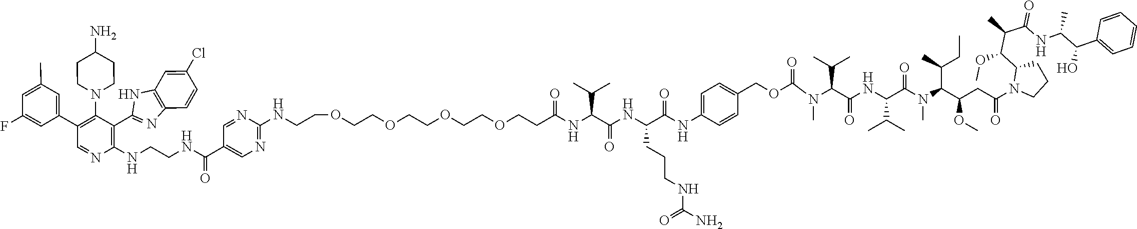 [SMILES: CC[C@H](C)[C@@H]([C@@H](CC(=O)N1CCC[C@H]1[C@H](OC)[C@@H](C)C(=O)N[C@H](C)[C@@H](O)c1ccccc1)OC)N(C)C(=O)[C@@H](NC(=O)[C@H](C(C)C)N(C)C(=O)OCc1ccc(NC(=O)[C@H](CCCNC(N)=O)NC(=O)[C@@H](NC(=O)CCOCCOCCOCCOCCNc2ncc(C(=O)NCCNc3ncc(-c4cc(C)cc(F)c4)c(N4CCC(N)CC4)c3-c3nc4ccc(Cl)cc4[nH]3)cn2)C(C)C)cc1)C(C)C] | |
| (Compound 180) | |
| 1-(S)-1-[N-(S)-1-{[(1S,2R)-4-[(S)-2-{(1R,2R)-2-[N-(1R,2S)-2-hydroxy-1-methyl-2-phenylethylcarbamoyl]-1-methoxypropyl}-1-pyrrolidinyl]- | 1829.0 |
| 1-[(S)-1-methylpropyl]-2-methoxy-4-oxobutyl]-N-methylcarbamoyl}-2-methylpropylcarbamoyl]-2-methylpropyl 1-methyl {p-[(S)-2-[(S)-2- | |
| [(R)-3-[N-2-(2-{N-2-[4-(4-amino-1-piperidyl)-3-(6-chloro-1H-1,3-benzimidazol-2-yl)-5-(3-fluoro-5-tolyl)-2- | |
| pyridylamino]ethylcarbamoyl}ethoxy)ethylcarbamoyl]-3-aminopropionylamino]-3-methylbutyrylamino]-5- | |
| ureidovalerylamino]phenyl}methanecarbamate | |
| 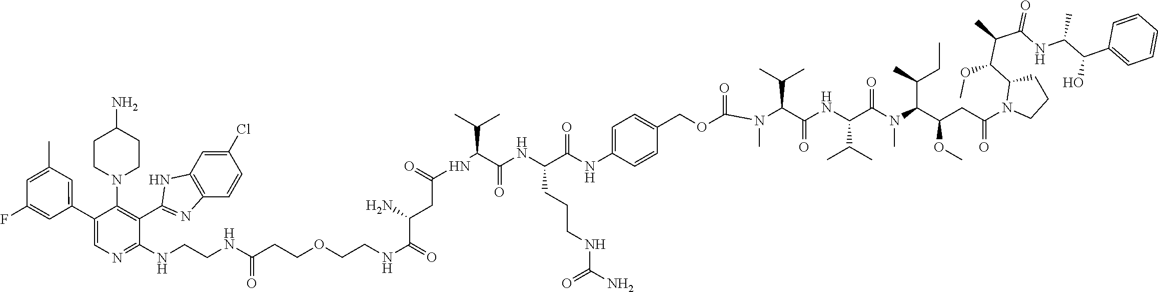 [SMILES: CC[C@H](C)[C@@H]([C@@H](CC(=O)N1CCC[C@H]1[C@H](OC)[C@@H](C)C(=O)N[C@H](C)[C@@H](O)c1ccccc1)OC)N(C)C(=O)[C@@H](NC(=O)[C@H](C(C)C)N(C)C(=O)OCc1ccc(NC(=O)[C@H](CCCNC(N)=O)NC(=O)[C@@H](NC(=O)C[C@@H](N)C(=O)NCCOCCC(=O)NCCNc2ncc(-c3cc(C)cc(F)c3)c(N3CCC(N)CC3)c2-c2nc3ccc(Cl)cc3[nH]2)C(C)C)cc1)C(C)C] | |
| (Compound 181) | |
| (2R,3S,4S,5R,6S)-6-{2-[N-(S)-1-[N-(S)-1-[N-p-({[(S)-1-[N-(S)-1-{[(1S,2R)-4-[(S)-2-{(1R,2R)-2-[N-(1R,2S)-2-hydroxy-1-methyl-2- | 2054.0 |
| phenylethylcarbamoyl]-1-methoxypropyl}-1-pyrrolidinyl]-1-[(S)-1-methylpropyl]-2-methoxy-4-oxobutyl]-N-methylcarbamoyl}-2- | |
| methylpropylcarbamoyl]-2-methylpropyl](methyl)(aminocarbonyloxy)}methyl)phenylcarbamoyl]-4-ureidobutylcarbamoyl]-2- | |
| methylpropylcarbamoyl]-5-[3-(2-{N-2-[4-(4-amino-1-piperidyl)-3-(6-chloro-1H-1,3-benzimidazol-2-yl)-5-(3-fluoro-5-tolyl)-2- | |
| pyridylamino]ethylcarbamoyl}ethoxy)propionylamino]phenoxy}-3,4,5-trihydroxytetrahydro-2H-pyran-2-carboxylic acid | |
| 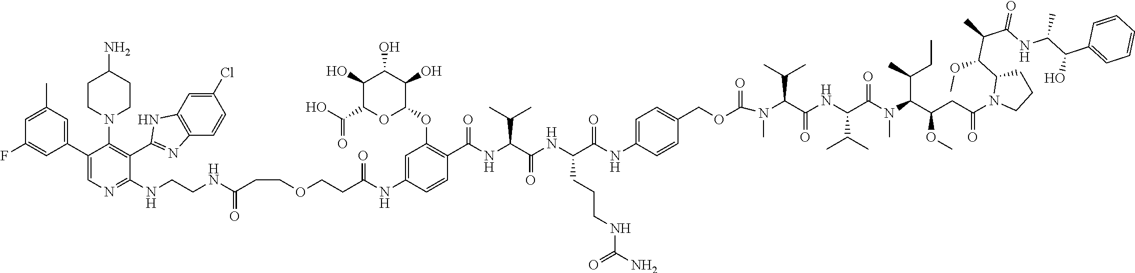 | |
| (Compound 182) | |
| (2R,3S,4S,5R,6S)-6-{2-[N-(S)-1-[N-(S)-1-[N-p-({[(S)-1-[N-(S)-1-{[(1S,2R)-4-[(S)-2-{(1R,2R)-2-[N-(1R,2S)-2-hydroxy-1-methyl-2- | 2186.1 |
| phenylethylcarbamoyl]-1-methoxypropyl}-1-pyrrolidinyl]-1-[(S)-1-methylpropyl]-2-methoxy-4-oxobutyl]-N-methylcarbamoyl}-2- | |
| methylpropylcarbamoyl]-2-methylpropyl](methyl)(aminocarbonyloxy)}methyl)phenylcarbamoyl]-4-ureidobutylcarbamoyl]-2- | |
| methylpropylcarbamoyl]-5-(14-{N-2-[4-(4-amino-1-piperidyl)-3-(6-chloro-1H-1,3-benzimidazol-2-yl)-5-(3-fluoro-5-tolyl)-2- | |
| pyridylamino]ethylcarbamoyl}-3,6,9,12-tetraoxatetradecylcarbonylamino)phenoxy}-3,4,5-trihydroxytetrahydro-2H-pyran-2-carboxylic acid | |
| 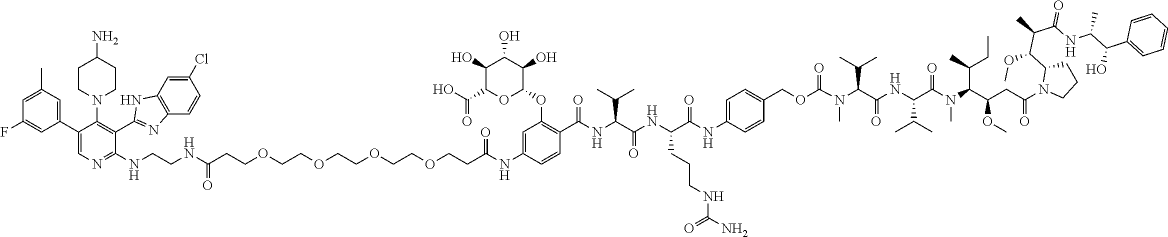 | |
| (Compound 183) | |
| 1-(S)-1-[N-(S)-1-{[(1S,2R)-4-[(S)-2-{(1R,2R)-2-[N-(1R,2S)-2-hydroxy-1-methyl-1-phenylethylcarbamoyl]-1-methoxypropyl}-1-pyrrolidinyl]- | 1920.0 |
| 1-[(S)-1-methylpropyl]-2-methoxy-4-oxobutyl]-N-methylcarbamoyl}-2-methylpropylcarbamoyl]-2-methylpropyl 1-methyl {p-[(S)-2-[(S)-2-[3- | |
| (N-2-aminoethylcarbamoyl)-5-[2-(2-{N-2-[4-(4-amino-1-piperidyl)-3-(6-chloro-1H-1,3-benzimidazol-2-yl)-5-(3-fluoro-5-tolyl)-2- | |
| pyridylamino]ethylcarbamoyl}ethoxy)ethylamino]benzylamino]-3-methylbutyrylamino]-5-ureidovalerylamino]phenyl}methanecarbamate | |
| 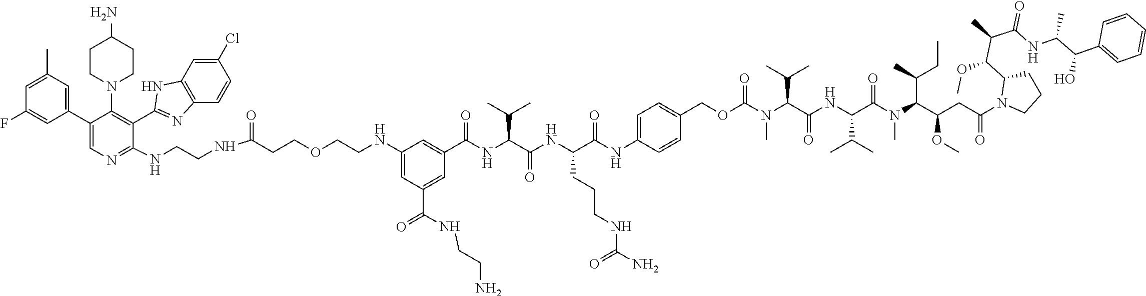 [SMILES: CC[C@H](C)[C@@H]([C@@H](CC(=O)N1CCC[C@H]1[C@H](OC)[C@@H](C)C(=O)N[C@H](C)[C@@H](O)c1ccccc1)OC)N(C)C(=O)[C@@H](NC(=O)[C@H](C(C)C)N(C)C(=O)OCc1ccc(NC(=O)[C@H](CCCNC(N)=O)NC(=O)[C@@H](NC(=O)c2cc(NCCOCCC(=O)NCCNc3ncc(-c4cc(C)cc(F)c4)c(N4CCC(N)CC4)c3-c3nc4ccc(Cl)cc4[nH]3)cc(C(=O)NCCN)c2)C(C)C)cc1)C(C)C] | |
| (Compound 184) | |
| {3-[N-(S)-1-[N-(S)-1-[N-p-({[(S)-1-[N-(S)-1-{[(1S,2R)-4-[(S)-2-{(1R,2R)-2-[N-(1R,2S)-2-hydroxy-1-methyl-2-phenylethylcarbamoyl]-1- | 1935.0 |
| methoxypropyl}-1-pyrrolidinyl]-1-[(S)-1-methylpropyl]-2-methoxy-4-oxobutyl]-N-methylcarbamoyl}-2-methylpropylcarbamoyl]-2- | |
| methylpropyl](methyl)(aminocarbonyloxy)}methyl)phenylcarbamoyl]-4-ureidobutylcarbamoyl]-2-methylpropylcarbamoyl]-5-[2-(2-{N-2-[4-(4- | |
| amino-1-piperidyl)-3-(6-chloro-1H-1,3-benzimidazol-2-yl)-5-(3-fluoro-5-tolyl)-2- | |
| pyridylamino]ethylcarbamoyl}ethoxy)ethylamino]benzylamino}acetic acid | |
| 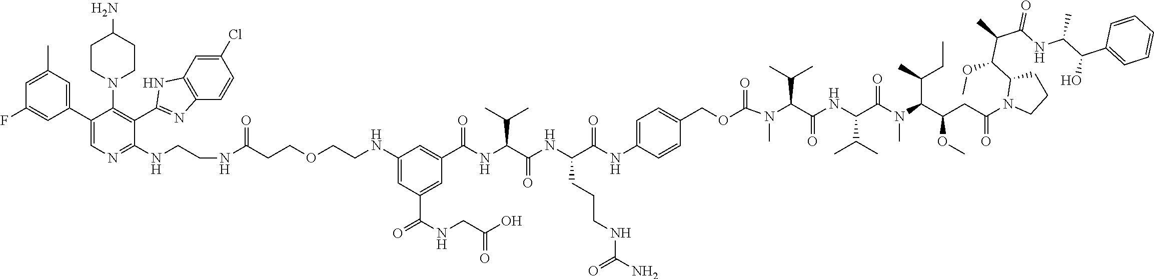 [SMILES: CC[C@H](C)[C@@H]([C@@H](CC(=O)N1CCC[C@H]1[C@H](OC)[C@@H](C)C(=O)N[C@H](C)[C@@H](O)c1ccccc1)OC)N(C)C(=O)[C@@H](NC(=O)[C@H](C(C)C)N(C)C(=O)OCc1ccc(NC(=O)[C@H](CCCNC(N)=O)NC(=O)[C@@H](NC(=O)c2cc(NCCOCCC(=O)NCCNc3ncc(-c4cc(C)cc(F)c4)c(N4CCC(N)CC4)c3-c3nc4ccc(Cl)cc4[nH]3)cc(C(=O)NCC(=O)O)c2)C(C)C)cc1)C(C)C] | |
| (Compound 185) | |
| 1-(S)-1-[N-(S)-1-{[(1S,2R)-4-[(S)-2-{(1R,2R)-2-[N-(1R,2S)-2-hydroxy-1-methyl-2-phenylethylcarbamoyl]-1-methoxypropyl}-1-pyrrolidinyl]- | 1934.0 |
| 1-[(S)-1-methylpropyl]-2-methoxy-4-oxobutyl]-N-methylcarbamoyl}-2-methylpropylcarbamoyl]-2-methylpropyl 1-methyl {p-[(S)-2-[(S)-2-{3- | |
| [2-(2-{N-2-[4-(4-amino-1-piperidyl)-3-(6-chloro-1H-1,3-benzimidazol-2-yl)-5-(3-fluoro-5-tolyl)-2- | |
| pyridylamino]ethylcarbamoyl}ethoxy)ethylamino]-5-[(carbamoylmethyl)carbamoyl]benzylamino}-3-methylbutyrylamino]-5- | |
| ureidovalerylamino]phenyl}methanecarbamate | |
| 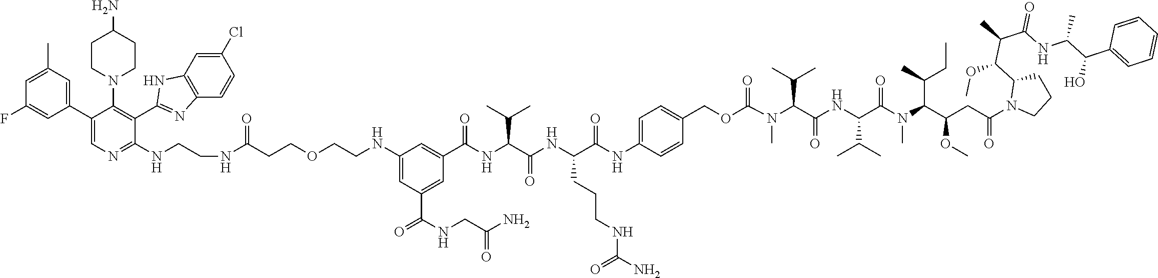 [SMILES: CC[C@H](C)[C@@H]([C@@H](CC(=O)N1CCC[C@H]1[C@H](OC)[C@@H](C)C(=O)N[C@H](C)[C@@H](O)c1ccccc1)OC)N(C)C(=O)[C@@H](NC(=O)[C@H](C(C)C)N(C)C(=O)OCc1ccc(NC(=O)[C@H](CCCNC(N)=O)NC(=O)[C@@H](NC(=O)c2cc(NCCOCCC(=O)NCCNc3ncc(-c4cc(C)cc(F)c4)c(N4CCC(N)CC4)c3-c3nc4ccc(Cl)cc4[nH]3)cc(C(=O)NCC(N)=O)c2)C(C)C)cc1)C(C)C] | |
| (Compound 186) | |
| 2-{3-[N-(S)-1-[N-(S)-1-[N-p-({[(S)-1-[N-(S)-1-{[(1S,2R)-4-[(S)-2-{(1R,2R)-2-[N-(1R,2S)-2-hydroxy-1-methyl-2-phenylethylcarbamoyl]-1- | 1984.9 |
| methoxypropyl}-1-pyrrolidinyl]-1-[(S)-1-methylpropyl]-2-methoxy-4-oxobutyl]-N-methylcarbamoyl}-2-methylpropylcarbamoyl]-2- | |
| methylpropyl](methyl)(aminocarbonyloxy)}methyl)phenylcarbamoyl]-4-ureidobutylcarbamoyl]-2-methylpropylcarbamoyl]-5-[2-(2-{N-2-[4-(4- | |
| amino-1-piperidyl)-3-(6-chloro-1H-1,3-benzimidazol-2-yl)-5-(3-fluoro-5-tolyl)-2- | |
| pyridylamino]ethylcarbamoyl}ethoxy)ethylamino]benzylamino}ethanesulfonic acid | |
| 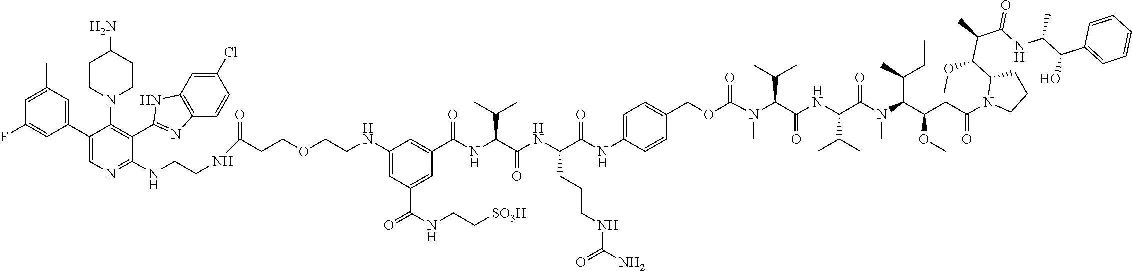 [SMILES: CC[C@H](C)[C@@H]([C@@H](CC(=O)N1CCC[C@H]1[C@H](OC)[C@@H](C)C(=O)N[C@H](C)[C@@H](O)c1ccccc1)OC)N(C)C(=O)[C@@H](NC(=O)[C@H](C(C)C)N(C)C(=O)OCc1ccc(NC(=O)[C@H](CCCNC(N)=O)NC(=O)[C@@H](NC(=O)c2cc(NCCOCCC(=O)NCCNc3ncc(-c4cc(C)cc(F)c4)c(N4CCC(N)CC4)c3-c3nc4ccc(Cl)cc4[nH]3)cc(C(=O)NCCS(=O)(=O)O)c2)C(C)C)cc1)C(C)C] | |
| (Compound 187) | |
| 2-{3-[N-(S)-1-[N-(S)-1-[N-p-({[(S)-1-[N-(S)-1-{[(1S,2R)-4-[(S)-2-{(1R,2R)-2-[N-(1R,2S)-2-hydroxy-1-methyl-2-phenylethylcarbamoyl]-1- | 1984.0 |
| methoxypropyl}-1-pyrrolidinyl]-1-[(S)-1-methylpropyl]-2-methoxy-4-oxobutyl]-N-methylcarbamoyl}-2-methylpropylcarbamoyl]-2- | |
| methylpropyl](methyl)(aminocarbonyloxy)}methyl)phenylcarbamoyl]-4-ureidobutylcarbamoyl]-2-methylpropylcarbamoyl]-5-[2-(2-{N-2-[4-(4- | |
| amino-1-piperidyl)-3-(6-chloro-1H-1,3-benzimidazol-2-yl)-5-(3-fluoro-5-tolyl)-2- | |
| pyridylamino]ethylcarbamoyl}ethoxy)ethylamino]benzylamino}ethanesulfonamide | |
| 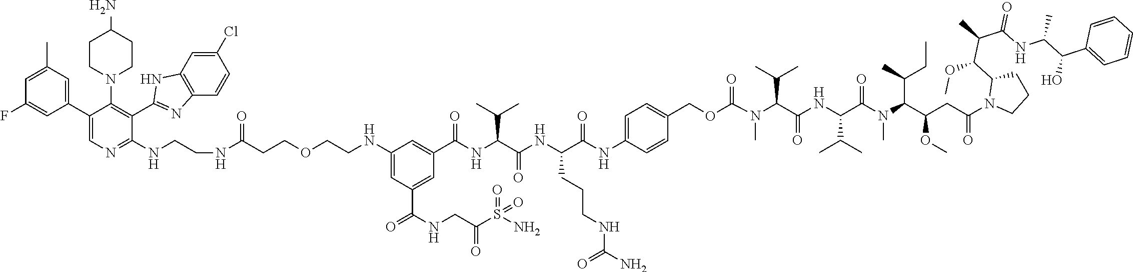 [SMILES: CC[C@H](C)[C@@H]([C@@H](CC(=O)N1CCC[C@H]1[C@H](OC)[C@@H](C)C(=O)N[C@H](C)[C@@H](O)c1ccccc1)OC)N(C)C(=O)[C@@H](NC(=O)[C@H](C(C)C)N(C)C(=O)OCc1ccc(NC(=O)[C@H](CCCNC(N)=O)NC(=O)[C@@H](NC(=O)c2cc(NCCOCCC(=O)NCCNc3ncc(-c4cc(C)cc(F)c4)c(N4CCC(N)CC4)c3-c3nc4ccc(Cl)cc4[nH]3)cc(C(=O)NCC(=O)S(N)(=O)=O)c2)C(C)C)cc1)C(C)C] | |
| (Compound 188) | |
| [(2-{3-[N-(S)-1-[N-(S)-1-[N-p-({[(S)-1-[N-(S)-1-{[(1S,2R)-4-[(S)-2-{(1R,2R)-2-[N-(1R,2S)-2-hydroxy-1-methyl-2-phenylethylcarbamoyl]-1- | 2036.0 |
| methoxypropyl}-1-pyrrolidinyl]-1-[(S)-1-methylpropyl]-2-methoxy-4-oxobutyl]-N-methylcarbamoyl}-2-methylpropylcarbamoyl]-2- | |
| methylpropyl](methyl)(aminocarbonyloxy)}methyl)phenylcarbamoyl]-4-ureidobutylcarbamoyl]-2-methylpropylcarbamoyl]-5-[2-(2-{N-2-[4-(4- | |
| amino-1-piperidyl)-3-(6-chloro-1H-1,3-benzimidazol-2-yl)-5-(3-fluoro-5-tolyl)-2- | |
| pyridylamino]ethylcarbamoyl}ethoxy)ethylamino]benzylamino}ethyl)carboxymethyl)amino]acetic acid | |
| 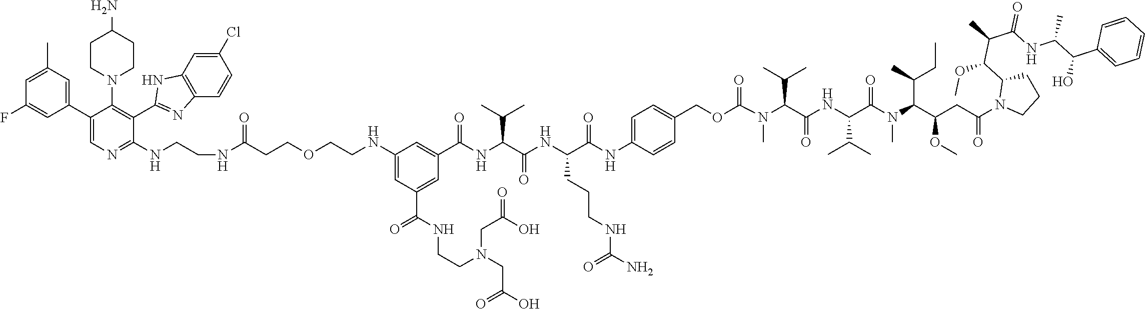 [SMILES: CC[C@H](C)[C@@H]([C@@H](CC(=O)N1CCC[C@H]1[C@H](OC)[C@@H](C)C(=O)N[C@H](C)[C@@H](O)c1ccccc1)OC)N(C)C(=O)[C@@H](NC(=O)[C@H](C(C)C)N(C)C(=O)OCc1ccc(NC(=O)[C@H](CCCNC(N)=O)NC(=O)[C@@H](NC(=O)c2cc(NCCOCCC(=O)NCCNc3ncc(-c4cc(C)cc(F)c4)c(N4CCC(N)CC4)c3-c3nc4ccc(Cl)cc4[nH]3)cc(C(=O)NCCN(CC(=O)O)CC(=O)O)c2)C(C)C)cc1)C(C)C] | |
| (Compound 189) | |
| (S)-4-(N-2-{2-[N-(S)-1-[N-(S)-1-[N-p-({[(S)-1-[N-(S)-1-{[(1S,2R)-4-[(S)-2-{(1R,2R)-2-[N-(1R,2S)-2-hydroxy-1-methyl-2- | 2248.1 |
| phenylethylcarbamoyl]-1-methoxypropyl}-1-pyrrolidinyl]-1-[(S)-1-methylpropyl]-2-methoxy-4-oxobutyl]-N-methylcarbamoyl}-2- | |
| methylpropylcarbamoyl]-2-methylpropyl](methyl)(aminocarbonyloxy)}methyl)phenylcarbamoyl]-4-ureidobutylcarbamoyl]-2- | |
| methylpropylcarbamoyl]ethoxy}ethylcarbamoyl)-4-[(S)-2-(14-{N-2-[4-(4-amino-1-piperidyl)-3-(6-chloro-1H-1,3-benzimidazol-2-yl)-5-(3- | |
| fluoro-5-tolyl)-2-pyridylamino]ethylcarbamoyl}-3,6,9,12-tetraoxatetradecylcarbonylamino)-4-carboxybutyrylamino]butyric acid | |
| 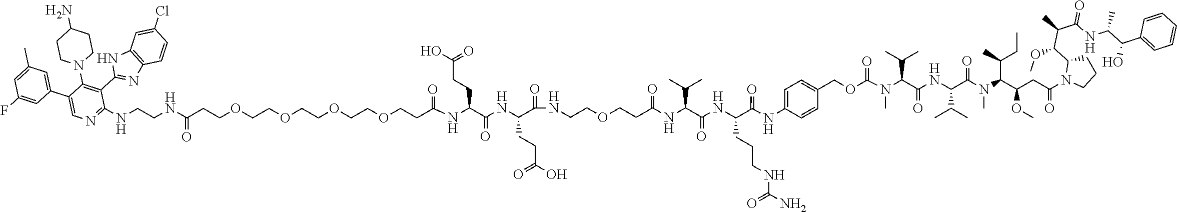 [SMILES: CC[C@H](C)[C@@H]([C@@H](CC(=O)N1CCC[C@H]1[C@H](OC)[C@@H](C)C(=O)N[C@H](C)[C@@H](O)c1ccccc1)OC)N(C)C(=O)[C@@H](NC(=O)[C@H](C(C)C)N(C)C(=O)OCc1ccc(NC(=O)[C@H](CCCNC(N)=O)NC(=O)[C@@H](NC(=O)CCOCCNC(=O)[C@H](CCC(=O)O)NC(=O)[C@H](CCC(=O)O)NC(=O)CCOCCOCCOCCOCCC(=O)NCCNc2ncc(-c3cc(C)cc(F)c3)c(N3CCC(N)CC3)c2-c2nc3ccc(Cl)cc3[nH]2)C(C)C)cc1)C(C)C] | |
| (Compound 190) | |
| (S)-4-(N-2-{2-[N-(S)-1-[N-(S)-1-[N-p-({[(S)-1-[N-(S)-1-{[(1S,2R)-4-[(S)-2-{(1R,2R)-2-[N-(1R,2S)-2-hydroxy-1-methyl-2- | 2116.1 |
| phenylethylcarbamoyl]-1-methoxypropyl}-1-pyrrolidinyl]-1-[(S)-1-methylpropyl]-2-methoxy-4-oxobutyl]-N-methylcarbamoyl}-2- | |
| methylpropylcarbamoyl]-2-methylpropyl](methyl)(aminocarbonyloxy)}methyl)phenylcarbamoyl]-4-ureidobutylcarbamoyl]-2- | |
| methylpropylcarbamoyl]ethoxy}ethylcarbamoyl)-4-[(S)-2-[3-(2-{N-2-[4-(4-amino-1-piperidyl)-3-(6-chloro-1H-1,3-benzimidazol-2-yl)-5-(3- | |
| fluoro-5-tolyl)-2-pyridylamino]ethylcarbamoyl}ethoxy)propionylamino]-4-carboxybutyrylamino]butyric acid | |
| 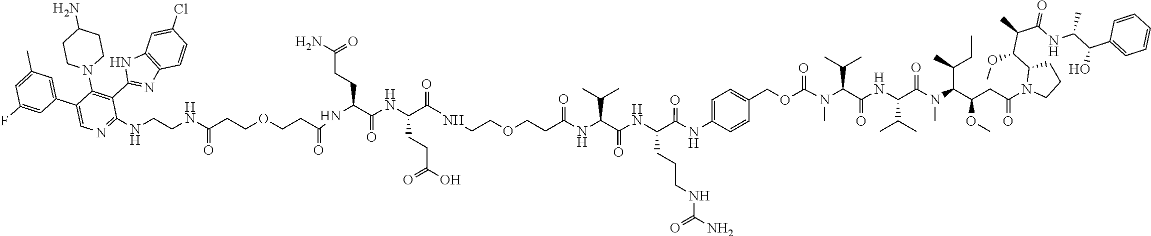 [SMILES: CC[C@H](C)[C@@H]([C@@H](CC(=O)N1CCC[C@H]1[C@H](OC)[C@@H](C)C(=O)N[C@H](C)[C@@H](O)c1ccccc1)OC)N(C)C(=O)[C@@H](NC(=O)[C@H](C(C)C)N(C)C(=O)OCc1ccc(NC(=O)[C@H](CCCNC(N)=O)NC(=O)[C@@H](NC(=O)CCOCCNC(=O)[C@H](CCC(=O)O)NC(=O)[C@H](CCC(N)=O)NC(=O)CCOCCC(=O)NCCNc2ncc(-c3cc(C)cc(F)c3)c(N3CCC(N)CC3)c2-c2nc3ccc(Cl)cc3[nH]2)C(C)C)cc1)C(C)C] | |
| (Compound 191) | |
| (S)-4-{N-5-[N-(S)-1-[N-(S)-1-[N-p-({[(S)-1-[N-(S)-1-{[(1S,2R)-4-[(S)-2-{(1R,2R)-2-[N-(1R,2S)-2-hydroxy-1-methyl-2- | 2246.2 |
| phenylethylcarbamoyl]-1-methoxypropyl}-1-pyrrolidinyl]-1-[(S)-1-methylpropyl]-2-methoxy-4-oxobutyl]-N-methylcarbamoyl}-2- | |
| methylpropylcarbamoyl]-2-methylpropyl](methyl)(aminocarbonyloxy)}methyl)phenylcarbamoyl]-4-ureidobutylcarbamoyl]-2- | |
| methylpropylcarbamoyl]pentylcarbamoyl}-4-[(S)-2-(14-{N-2-[4-(4-amino-1-piperidyl)-3-(6-chloro-1H-1,3-benzimidazol-2-yl)-5-(3-fluoro-5- | |
| tolyl)-2-pyridylamino]ethylcarbamoyl}-3,6,9,12-tetraoxatetradecylcarbonylamino)-4-carboxybutyrylamino]butyric acid | |
| 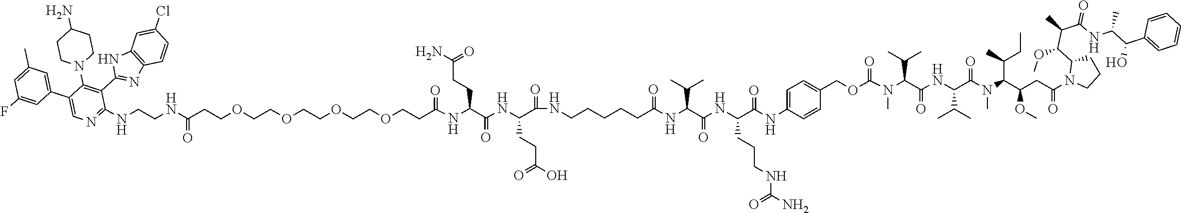 [SMILES: CC[C@H](C)[C@@H]([C@@H](CC(=O)N1CCC[C@H]1[C@H](OC)[C@@H](C)C(=O)N[C@H](C)[C@@H](O)c1ccccc1)OC)N(C)C(=O)[C@@H](NC(=O)[C@H](C(C)C)N(C)C(=O)OCc1ccc(NC(=O)[C@H](CCCNC(N)=O)NC(=O)[C@@H](NC(=O)CCCCCNC(=O)[C@H](CCC(=O)O)NC(=O)[C@H](CCC(N)=O)NC(=O)CCOCCOCCOCCOCCC(=O)NCCNc2ncc(-c3cc(C)cc(F)c3)c(N3CCC(N)CC3)c2-c2nc3ccc(Cl)cc3[nH]2)C(C)C)cc1)C(C)C] | |
| (Compound 192) | |
| 1-(S)-1-[N-(S)-1-{[(1S,2R)-4-[(S)-2-{(1R,2R)-2-[N-(1R,2S)-2-hydroxy-1-methyl-2-phenylethylcarbamooyl]-1-methoxypropyl}-1-pyrrolidinyl]- | 1875.0 |
| 1-[(S)-1-methylpropyl]-2-methoxy-4-oxobutyl]-N-methylcarbamoyl}-2-methylpropylcarbamoyl]-2-methylpropyl 1-methyl {p-[(S)-2-[(S)-2- | |
| (14-{3-[4-(4-amino-1-piperidyl)-3-(6-chloro-1H-1,3-benzimidazol-2-yl)-5-(3-fluoro-5-tolyl)-2-pyridylamino]propionylamino}-3,6,9,12- | |
| tetraoxatetradecylcarbonylamino)-3-methylbutyrylamino]-5-ureidovalerylamino]phenyl}methanecarbamate | |
| 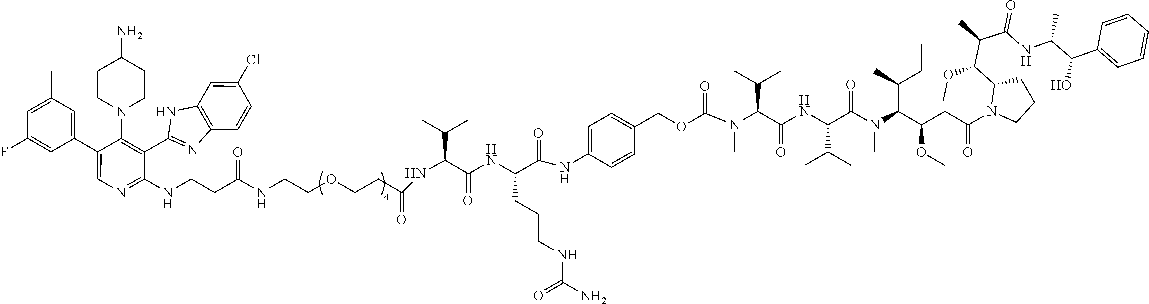 | |
| (Compound 196) | |
| 1-(S)-1-[N-(S)-1-{[(1S,2R)-4-[(S)-2-{(1R,2R)-2-[N-(1R,2S)-2-hydroxy-1-methyl-2-phenylethylcarbamoyl]-1-methoxypropyl}-1-pyrrolidinyl]- | 1889.0 |
| 1-[(S)-1-methylpropyl]-2-methoxy-4-oxobutyl]-N-methylcarbamoyl}-2-methylpropylcarbamoyl]-2-methylpropyl 1-methyl {p-[(S)-2-[(S)-2- | |
| (14-{N-3-[4-(4-amino-1-piperidyl)-3-(6-chloro-1H-1,3-benzimidazol-2-yl)-5-(3-fluoro-5-tolyl)-2-pyridylamino]propylcarbamoyl}-3,6,9,12- | |
| tetraoxatetradecylcarbonylamino)-3-methylbutyrylamino]-5-ureidovalerylamino]phenyl}methanecarbamate | |
| 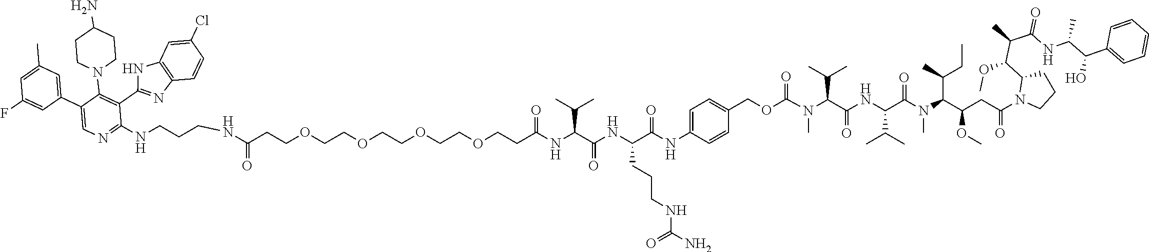 [SMILES: CC[C@H](C)[C@@H]([C@@H](CC(=O)N1CCC[C@H]1[C@H](OC)[C@@H](C)C(=O)N[C@H](C)[C@@H](O)c1ccccc1)OC)N(C)C(=O)[C@@H](NC(=O)[C@H](C(C)C)N(C)C(=O)OCc1ccc(NC(=O)[C@H](CCCNC(N)=O)NC(=O)[C@@H](NC(=O)CCOCCOCCOCCOCCC(=O)NCCCNc2ncc(-c3cc(C)cc(F)c3)c(N3CCC(N)CC3)c2-c2nc3ccc(Cl)cc3[nH]2)C(C)C)cc1)C(C)C] | |
| (Compound 197) | |
| (R)-2-hydroxy-3-[5-(3-{2-[N-methyl({N-2-[4-(4-amino-1-piperidyl)-3-(6-chloro-1H-1,3-benzimidazol-2-yl)-5-(3-fluoro-5-tolyl)-2- | 2064.0 |
| pyridylamino]ethylcarbamoyl}methyl)carbamoyl]ethoxy}propionylamino)-3-[N-(S)-2-methyl-1-[N-(S)-1-[N-p-({methyl[(S)-2-methyl-1-[N- | |
| (S)-1-{[(1S,2R)-2-methoxy-4-[(S)-2-[(1R,2R)-1-methoxy-2-[N-(1R,2S)-2-hydroxy-1-methyl-2-phenylethylcarbamoyl]propyl]-1-pyrrolidinyl]- | |
| 1-[(S)-1-methylpropyl]-4-oxobutyl]-N-methylcarbamoyl}-2-methylpropylcarbamoyl]propyl](aminocarbonyloxy)}methyl)phenylcarbamoyl]-4- | |
| ureidobutylcarbamoyl]propylcarbamoyl]benzylamino]propionic acid | |
| 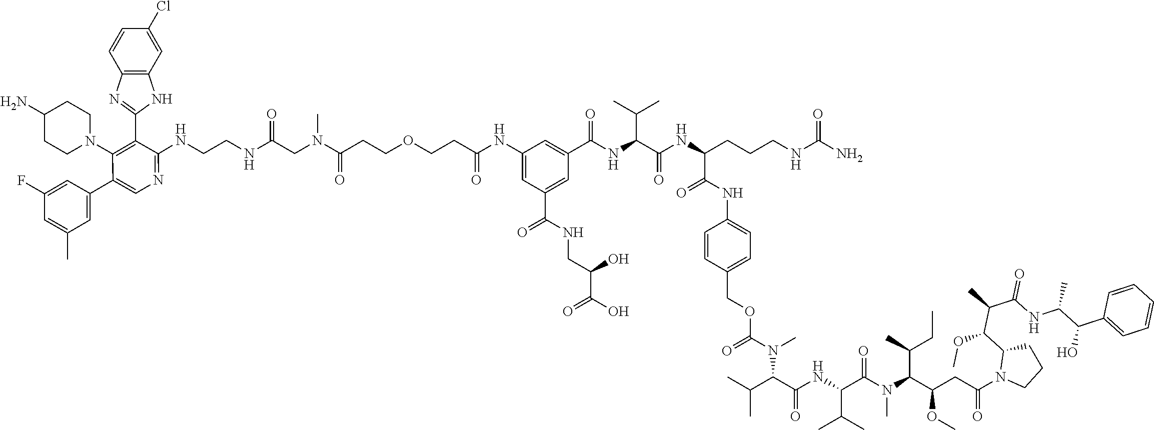 [SMILES: CC[C@H](C)[C@@H]([C@@H](CC(=O)N1CCC[C@H]1[C@H](OC)[C@@H](C)C(=O)N[C@H](C)[C@@H](O)c1ccccc1)OC)N(C)C(=O)[C@@H](NC(=O)[C@H](C(C)C)N(C)C(=O)OCc1ccc(NC(=O)[C@H](CCCNC(N)=O)NC(=O)[C@@H](NC(=O)c2cc(NC(=O)CCOCCC(=O)N(C)CC(=O)NCCNc3ncc(-c4cc(C)cc(F)c4)c(N4CCC(N)CC4)c3-c3nc4ccc(Cl)cc4[nH]3)cc(C(=O)NC[C@@H](O)C(=O)O)c2)C(C)C)cc1)C(C)C] | |
| (Compound 244) | |
| (12S,25S)-12-(4-(4-((6S,9S,12S)-1-amino-6-((4-((5S,8S,11S,12R)-11-((S)-sec-butyl)-12-(2-((S)-2-((1R,2R)-3-(((1S,2R)-1-hydroxy-1- | 3679.0 |
| phenylpropan-2-yl)amino)-1-methoxy-2-methyl-3-oxopropyl)pyrrolidin-1-yl)-2-oxoethyl)-5,8-diisopropyl-4,10-dimethyl-3,6,9-trioxo-2,13- | |
| dioxa-4,7,10-triazatetradecyl)phenyl)carbamoyl)-12-(2-carboxyethyl)-9-isopropyl-1,8,11,14,24-pentaoxo-17,20-dioxa-2,7,10,13,23- | |
| pentaazahexacosan-26-yl)-1H-1,2,3-triazol-1-yl)butyl)-1-((4-(4-aminopiperidin-1-yl)-3-(6-chloro-1H-benzo[d]imidazol-2-yl)-5-(3-fluoro-5- | |
| methylphenyl)pyridin-2-yl)amino)-25-(((S)-1-(((S)-1-((4-((5S,8S,11S,12R)-11-((S)-sec-butyl)-12-(2-((S)-2-((1R,2R)-3-(((1S,2R)-1-hydroxy-1- | |
| phenylpropan-2-yl)amino)-1-methoxy-2-methyl-3-oxopropyl)pyrrolidin-1-yl)-2-oxoethyl)-5,8-diazaspiro-4,10-dimethyl-3,6,9-trioxo-2,13- | |
| dioxa-4,7,10-triazatetradecyl)phenyl)amino)-1-oxo-5-ureidopentan-2-yl)amino)-3-methyl-1-oxobutan-2-yl)carbamoyl)-4,10,13,23-tetraoxo- | |
| 7,17,20-trioxa-3,11,14,24-tetraazaoctacosan-28-oic acid | |
| 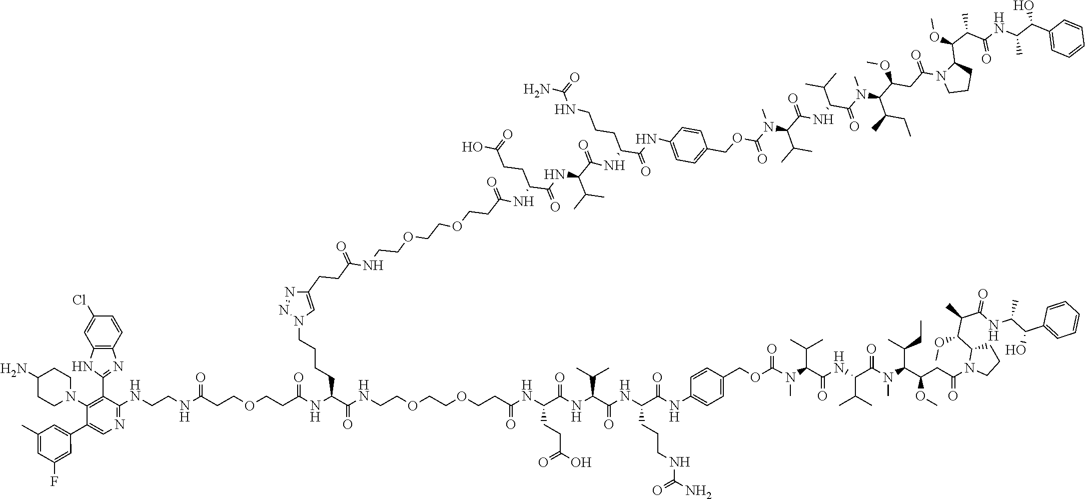 | |
| (Compound 245) | |

### Biological Assays

### Example F: SSTR Assay

General overview: All five SSTR subtypes are Gi coupled G-protein coupled receptors (GPCRs) that lead to decreases in intracellular cyclic AMP (cAMP) when activated by an agonist. Therefore, measurement of intracellular cAMP levels can be used to assess whether compounds of the invention are agonists of SSTR subtypes (John Kelly, Troy Stevens, W. Joseph Thompson, and Roland Seifert, Current Protocols in Pharmacology. 2005, 2.2.1-2.2). One example of an intracellular cAMP assay is described below.

### Functional Assay for SSTR2 Agonists

Four days prior to the assay, 2,000 Chinese hamster ovary cells (CHO-K1, ATCC #CCL-61) stably expressing the human somatostatin receptor subtype 2 are plated in each well of a 96-well tissue culture-treated plate in Ham's F12 growth media (ThermoFisher #10-080-CM) supplemented with 10% donor bovine serum (Gemini Bio-Products #100-506), 100 U/mL penicillin; 100 ug/mL streptomycin, 2 mM L-glutamine (Gemini Bio-Products #400-110) and 0.2 mg/mL hygromycin B (GoldBio #31282-04-9). The cells are cultured at 37° C., 5% CO₂ and 95% humidity. cAMP is measured with HTRF dynamic cAMP assay (Cisbio. #62AM5PEJ) per manufacturer's instructions. On the day of the assay, the media is aspirated and the cells are treated with 50 μL of stimulation buffer supplemented with 10.2 mM 3-Isobutyl-1-methylxanthine (IBMX, Millipore Sigma #I5879) and 1.6 μM NKH477 (Tocris #1603), plus various dilutions of NDCs of the present invention. The cells are incubated for 20 minutes at 37° C. (the final concentration of the compounds of the invention are typically 0-10,000 nM). The cells are treated with 50 μL of lysis buffer (HRTF cAMP kit, Cisbio) and incubated at room temperature for 30 minutes with rotary shaking at 600 rpm, and then diluted with 150 μL of stimulation buffer and shaken for an additional five minutes at 300 rpm. The lysate is transferred to 384-well plates and incubated for 1-24 hours at room temperature, and cAMP accumulation is detected by d2-labeled cAMP and Anti-cAMP-Cryptate. The time-resolved fluorescent signal is read using a m1000 Pro (Tecan) or CLARIOStar (BMG Labtech) microplate reader, where the samples are excited with light at 340 nm and emission light is measured at 620 nm and 665 nm. The data is expressed as a calculation of the fluorescence ratio (665 nm/620 nm). The intracellular cAMP concentrations are calculated by regression to a standard curve and are plotted vs, the concentration of the compounds of the invention. The EC₅₀ of the compounds are calculated using standard methods. All data manipulations are in GraphPad Prism v9 (GraphPad, San Diego, CA).

### Example G: Internalization Assay Protocol

NDCs of the present invention were assessed in vitro using the human PathHunter CHO-K1 SST2 β-arrestin2 and PathHunter CHO-K1 SST2 Internalization Assays from DiscoverX. The CHO-K cells were seeded in 20 μLs of cell culture medium in a white-walled 384 well tissue culture-compatible plate at a density of 2500 cells/well. After a 48-hour incubation, cells were treated with 5 μLs of a 1:3 serially diluted SST2 NDC prepared at 5× concentration with a final top compound concentration of 10 uM for 90 min at 37 C and in 5% CO₂. 11 dilutions were tested as well as one treatment that was compound free. After incubation, the media containing treatments were discarded and 20 μL of a working solution of Beta-Glo from Promega Beta-Go Assay System was added to each well. The plate was then incubated for 60 min at room temperature in the dark. Luminescence was measured using a Tecan plate reader. IC₅₀ curves were generated using the nonlinear regression analysis (four-parameters) with GraphPad Prism 9. Results were the average of duplicates and are corrected for background.

### Example H: H524 Assay Protocol

NDCs of the present invention were assessed in an in vitro assay evaluating the inhibition of cell proliferation. NCI-H524 (ATCC) human small cell lung cancer cells were plated in 96-well V-bottom plates (Costar) at a concentration of 5,000 cells/well. 24 h later, the cells were treated for 2 h with a NDC. NDC starting dose was 1 uM and two-fold serial dilutions were done for a total of 11 points. After 2 h of treatment, cells were spun down, and the drug-containing media was removed, and fresh complete media was added and used to resuspend the cells, which were spun again. After removal of the media, the cells were resuspended in complete medium and incubated for an additional 70 h. Cell proliferation was assessed using the CellTiter Glo Assay according to the standard protocol (Promega). Luminescence was measured using a TECAN plate reader. Percent proliferation inhibition was calculated using the following formula: % inhibition=(Luminescence Control−Luminescence Treatment)/Luminescence Control*100. IC₅₀ curves were generated using the nonlinear regression analysis (four-parameter) with GraphPad Prism 9. Illustrative biological activity of compounds is demonstrated in the following Tables.

TABLE 1
Representative Activity

| | hSST2 cAMP | Internalization | H524 |
|---|---|---|---|
| Compound | Avg EC₅₀ (nM) | EC₅₀ (nM) | EC₅₀ (nM) |
| 142 | 0.37 | 6.3 | 790 |
| 176 | 0.28 | 3.7 | 620 |
| 57 | 0.31 | 6.6 | 1000 |
| 177 | 0.23 | 11 | 2300 |
| 178 | 0.27 | 5.6 | 620 |
| 179 | 0.54 | 9.8 | 370 |
| 180 | 0.52 | 16 | 680 |
| 92 | 0.2 | 2.5 | 680 |
| 181 | 0.38 | 3.4 | 190 |
| 130 | 0.25 | 4.4 | 3800 |
| 84 | 0.28 | 3.8 | 580 |
| 182 | 0.093 | 11 | 2300 |
| 183 | 0.074 | 3.1 | 1000 |
| 85 | 0.72 | 3.6 | 73 |
| 184 | 0.063 | 1.8 | 240 |
| 185 | 0.19 | 2.6 | 1100 |
| 186 | 0.26 | 2.7 | 470 |
| 187 | 0.5 | 3.7 | 1800 |
| 188 | 0.34 | 2.7 | 670 |
| 131 | 0.34 | 4.5 | 1600 |
| 2 | 0.57 | 8.6 | 2500 |
| 189 | 0.4 | 7.6 | 2200 |
| 3 | 0.19 | 6.8 | 680 |
| 86 | 0.068 | 4.1 | 1000 |
| 190 | 0.33 | 14 | 3200 |
| 87 | 0.1 | 4.6 | 600 |
| 88 | 0.25 | 3.1 | 84 |
| 4 | 0.11 | 3.8 | 740 |
| 132 | 0.26 | 3.2 | 410 |
| 191 | 1.9 | 38 | 1700 |
| 89 | 0.57 | 3.6 | 1100 |
| 90 | 0.66 | 5.5 | 480 |
| 91 | 0.46 | 2.5 | 1000 |
| 5 | 0.29 | 2.2 | 520 |
| 6 | 0.21 | 3.5 | 7300 |
| 58 | 0.26 | 14 | 910 |
| 133 | 0.097 | 5.3 | 1900 |
| 134 | 0.11 | 2.1 | 1200 |
| 135 | 0.42 | 3.6 | 650 |
| 136 | 0.29 | 3.5 | 540 |
| 137 | 0.27 | 2.8 | 850 |
| 59 | 0.25 | 2 | 260 |
| 192 | 0.37 | 12 | 1300 |
| 143 | 0.4 | n.d. | n.d. |
| 7 | 0.26 | n.d. | n.d. |
| 60 | 0.17 | n.d. | n.d. |
| 32 | 0.32 | 2.3 | 0.48 |
| 33 | 0.15 | 2.5 | 7 |
| 64 | 0.12 | 2.9 | 11 |
| 9 | 0.38 | 2.5 | 4.3 |
| 65 | 0.074 | 3.4 | 17 |
| 10 | 0.21 | 2.7 | 0.76 |
| 11 | 0.33 | 10 | 14 |
| 138 | 0.072 | 4.4 | 20 |
| 139 | 0.1 | 4.9 | 30 |
| 144 | 0.25 | 21 | 81 |
| 145 | 0.38 | 17 | 78 |
| 146 | 0.29 | 9.9 | 62 |
| 34 | 0.12 | 4.4 | 1.6 |
| 140 | 0.15 | 2.9 | 6.6 |
| 147 | 0.56 | 23 | 35 |
| 12 | 0.24 | 6.9 | 3.9 |
| 141 | 0.1 | 8.8 | 20 |
| 66 | 0.14 | 5.6 | 6.8 |
| 13 | 0.16 | 3.2 | 11 |
| 14 | 0.037 | 4.6 | 19 |
| 67 | 0.086 | 5.5 | 85 |
| 148 | 0.18 | 8.9 | 30 |
| 93 | 0.28 | 33 | 120 |
| 15 | 0.27 | 17 | 91 |
| 1 | 0.25 | 18 | 15 |
| 16 | 0.083 | 7.8 | 160 |
| 69 | 0.16 | 5.6 | 36 |
| 149 | 0.033 | 12 | 100 |
| 150 | 0.083 | 8.3 | 53 |
| 35 | 0.1 | 5 | 16 |
| 17 | 0.44 | 3.8 | 5.4 |
| 71 | 0.14 | 7.5 | 100 |
| 72 | 0.11 | 4.7 | 40 |
| 18 | 0.097 | 6.4 | 7.8 |
| 19 | 0.07 | 8.1 | 28 |
| 20 | 0.093 | 7 | 29 |
| 21 | 0.096 | 7.6 | 13 |
| 40 | 0.2 | 8.7 | 1.7 |
| 22 | 0.17 | 17 | 22 |
| 23 | 0.29 | 20 | 32 |
| 24 | 0.2 | 20 | 100 |
| 25 | 0.29 | 6.5 | 41 |
| 26 | 0.3 | 12 | 17 |
| 56 | 0.81 | 68 | 26 |
| 73 | 0.06 | 3.1 | 110 |
| 74 | 0.19 | 4.3 | 17 |
| 75 | 0.21 | 2.4 | 440 |
| 76 | 0.075 | 4.7 | 12 |
| 77 | 0.092 | 4.2 | 130 |
| 27 | 0.12 | 7.2 | 190 |
| 28 | 0.074 | 11 | 120 |
| 29 | 0.076 | 8.5 | 2.6 |
| 41 | 0.081 | 9 | 40 |
| 78 | 0.22 | 8.7 | 350 |
| 42 | 0.1 | 3.8 | 25 |
| 43 | 0.24 | 6.6 | 1.8 |
| 44 | 0.085 | 4.5 | 4.8 |
| 45 | 1.3 | n.d. | n.d. |
| 46 | 0.23 | n.d | n.d |
| 47 | 0.31 | n.d. | n.d. |
| 154 | 1.6 | 7.7 | 39 |
| 155 | 0.44 | 10 | 300 |
| 108 | 0.18 | 8.2 | 52 |
| 109 | 1.1 | 35 | 200 |
| 110 | 1 | 23 | 120 |
| 156 | 0.28 | 14 | 140 |
| 111 | 0.046 | 8.7 | 19 |
| 112 | 0.12 | 8.4 | 10 |
| 113 | 110 | 1300 | 2000 |
| 114 | 85 | 850 | 4600 |
| 80 | 180 | 920 | 540 |
| 115 | 0.075 | 5.9 | 5.1 |
| 116 | 0.19 | 14 | 21 |
| 117 | 0.46 | 19 | 58 |
| 157 | 0.45 | 20 | 95 |
| 118 | 0.24 | 9.3 | 22 |
| 81 | 0.094 | 10 | 2 |
| 119 | 0.11 | 11 | 34 |
| 120 | 0.088 | 5.2 | 20 |
| 121 | 0.14 | 5.3 | 19 |
| 158 | 0.15 | 5.1 | 40 |
| 82 | 0.26 | 13 | 6.6 |
| 122 | 0.19 | 8.8 | 27 |
| 159 | 1.2 | 23 | 130 |
| 160 | 0.25 | 7.4 | 100 |
| 161 | 0.32 | 9.1 | 63 |
| 162 | 0.27 | 4.7 | 60 |
| 123 | 0.2 | 2.3 | 8.4 |
| 163 | 0.15 | 6.3 | 67 |
| 164 | 0.35 | 4.1 | 27 |
| 165 | 0.5 | 10 | 90 |
| 79 | 0.12 | 5.1 | 7.7 |
| 166 | 0.29 | 4.5 | 18 |
| 167 | 0.24 | 13 | 150 |
| 168 | 0.17 | 5.8 | 34 |
| 169 | 0.1 | 10 | 32 |
| 170 | 0.31 | 12 | 22 |
| 171 | 0.33 | 8.3 | 14 |
| 172 | 0.33 | 6.3 | 22 |
| 173 | 0.24 | 19 | 52 |
| 83 | 0.53 | 7.0 | 310 |
| 49 | 0.14 | 3.4 | 9.4 |
| 50 | 0.13 | 6.8 | 36 |
| 51 | 0.092 | 4.4 | 28 |
| 175 | 0.41 | 18 | 56 |
| 52 | 0.14 | 3.5 | 17 |
| 53 | 0.48 | n.d. | 200 |
| 54 | 0.26 | 2.1 | 20 |
| 55 | 0.25 | 2.0 | 11 |
| 125 | 0.59 | 48 | 580 |
| 126 | 0.41 | 39 | 340 |
| 127 | 1.6 | 290 | 170 |
| 128 | 200 | 3899 | 3200 |
| 129 | 190 | 960 | 3000 |
| 196 | 0.29 | 8.6 | 28 |
| 194 | 0.16 | 7.6 | 21 |
| 197 | 0.27 | 6.5 | 12 |
| 195 | 0.35 | 4.1 | 8.5 |
| 193 | 0.87 | 3.8 | 32 |
| 239 | 0.16 | 4.9 | 12 |
| 244 | 0.26 | 4 | 55 |
| 240 | 0.12 | 6 | 3100 |
| 241 | 0.19 | 4.6 | 2900 |
| 242 | 0.045 | 2.5 | 70 |
| 243 | 0.082 | 3.2 | 17 |
| 198 | 0.082 | 3.5 | 32 |
| 199 | 0.071 | 4.1 | 8.9 |
| 200 | 0.18 | 3.9 | 23 |
| 20 | 0.1 | 4.2 | 21 |
| 202 | 0.14 | 4.6 | 14 |
| 203 | 0.11 | 7.7 | 71 |
| 204 | 0.14 | 9.2 | 30 |
| 206 | 0.12 | 4.5 | 14 |
| 207 | 0.23 | 5.9 | 8.4 |
| 208 | 0.062 | 3.1 | 8.9 |
| 209 | 0.11 | 2.7 | 1.5 |
| 210 | 0.051 | 2.8 | 2300 |
| 211 | 0.19 | 3.2 | 89 |
| 212 | 0.088 | 4.5 | 20 |
| 213 | 0.089 | 3.4 | 9.2 |
| 214 | 0.11 | 4 | 8.9 |
| 205 | 0.086 | 5.2 | 37 |
| 215 | 0.14 | 7.8 | 5. |
| 216 | 0.076 | 3 | 8.6 |
| 217 | 0.093 | 6 | 6.9 |
| 218 | 0.07 | 2.8 | 7 |
| 219 | 0.099 | 2.7 | 7.5 |
| 220 | 0.077 | 3.1 | 7.6 |
| 221 | 0.15 | 8.8 | 27 |
| 245 | 11 | 440 | 140 |
| 222 | 0.11 | 4.3 | 1.9 |
| 223 | 0.054 | 2.8 | 9.2 |
| 246 | 0.1 | 1.5 | 74 |
| 224 | 0.068 | 3.2 | 27 |
| 247 | 0.19 | 1.4 | 18 |
| 225 | 0.26 | 1.9 | 18 |
| 226 | 0.28 | 9.3 | 4.3 |
| 227 | 0.078 | 3.1 | 11 |
| 228 | 0.26 | 10 | 36 |
| 229 | 0.1 | 4.8 | 4 |
| 230 | 0.095 | 4.2 | 14 |
| 231 | 0.33 | 12 | 49 |
| 232 | 0.15 | 5.2 | 5 |
| 233 | 1 | 18 | 500 |
| 234 | 0.55 | 6.6 | 140 |
| 235 | 0.11 | 4.4 | 14 |
| 236 | 0.14 | 5.1 | 9.9 |
| 237 | 0.15 | 8.4 | 8.2 |
| 238 | 0.12 | 4.7 | 2.9 |
| 248 | 0.14 | 2.5 | 3.2 |
| 249 | 0.086 | 4.5 | 9.8 |

The examples and embodiments described herein are for illustrative purposes only and various modifications or changes suggested to persons skilled in the art are to be included within the spirit and purview of this application and scope of the appended claims.

1.-54. (canceled)

55. A compound that has the structure of Formula (I), or a pharmaceutically acceptable salt thereof: 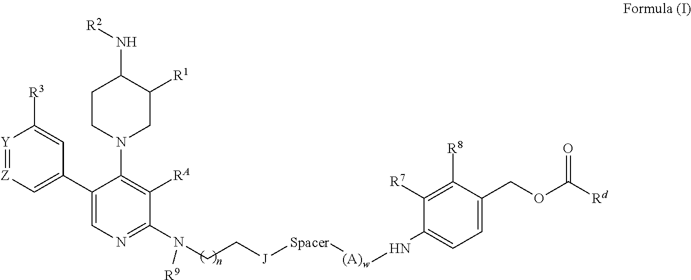
wherein:
J is a bond or NR⁶,
n is an integer selected from 1-5;
R^{A} is 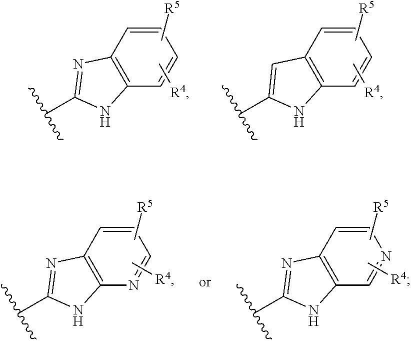
Y is N or CH;
Z is N or CR¹⁰;
R¹ is H;
R² is H or C₁-C₆ alkyl; or
R¹ and R², together with the N and C to which they are attached, combine to form a heterocycloalkyl;
R³ is C₁-C₆ alkyl or C₁-C₆ alkoxy;
R⁴ is H, halogen, CN, or C₁-C₆ alkoxy;
R⁵ is H, halogen, or CN;
R⁶ is H or C₁-C₆ alkyl;
R⁹ is H or C₁-C₆ alkyl;
R¹⁰ is H or halogen;
Spacer is a spacer group;
each A is independently selected from natural or unnatural amino acids, wherein any free amine of an amino acid is optionally independently substituted with CH₃;
w is an integer selected from 1-6;
R⁷ is H, C₁-C₆ haloalkyl, OR^{7a}, 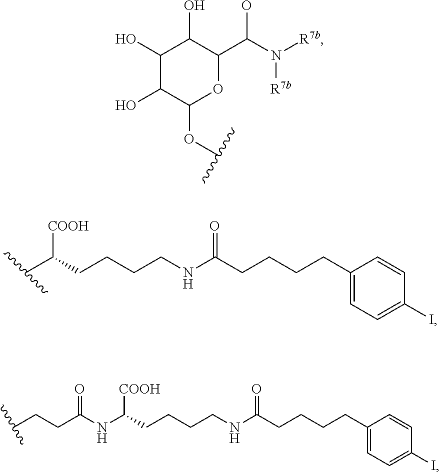
C₁-C₆ alkyl optionally substituted with COOH;
R^{7a} is H, or C₁-C₆ alkyl optionally substituted with amino, COOH, or OH;
R^{7b} is H, C₁-C₂₀ alkyl optionally substituted with OR^{7b1}, COOR^{7b1}, NR^{7b1}₂, or SO₃R^{7b1}, (CH₂)₂NHC(═O)—R^{7b2}, 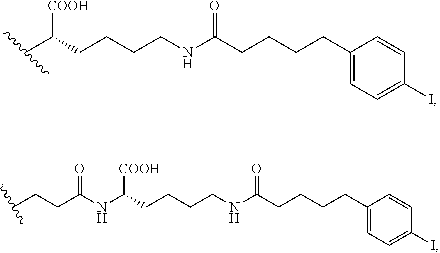 [SMILES: *[C@@H](CCCCNC(=O)CCCCc1ccc(I)cc1)C(=O)O ; *CCC(=O)N[C@@H](CCCCNC(=O)CCCCc1ccc(I)cc1)C(=O)O]
(CH₂CH₂O)ₓCH₂CH₂COOH, 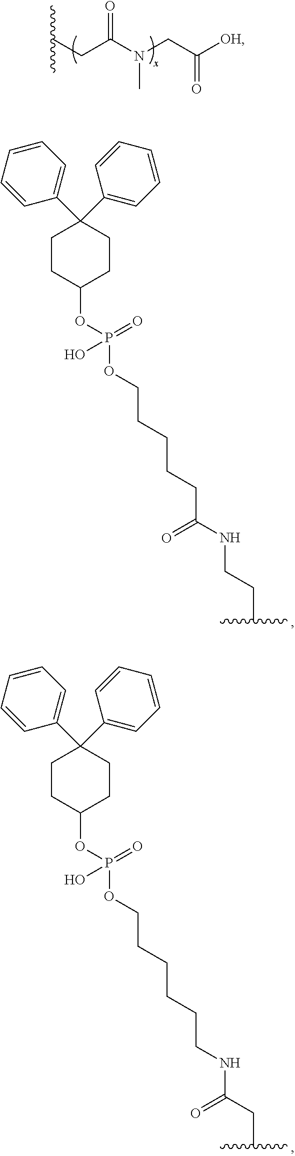 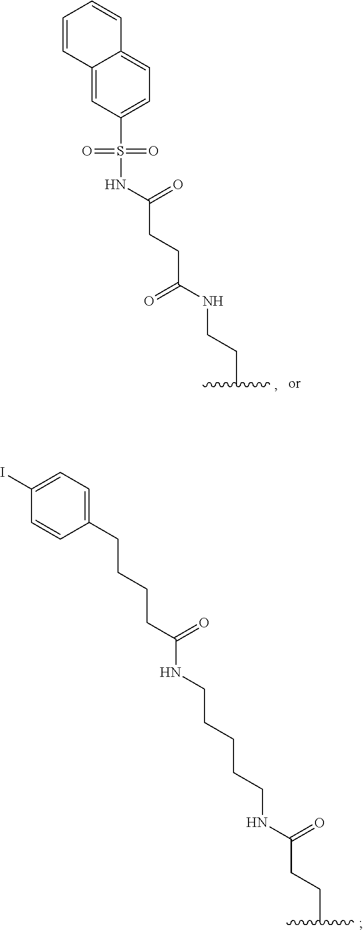 [SMILES: *CCNC(=O)CCC(=O)NS(=O)(=O)c1ccc2ccccc2c1 ; *CCC(=O)NCCCCCNC(=O)CCCCc1ccc(I)cc1]
each R^{7b1} is independently selected from H and C₁-C₆ alkyl;
R^{7b2} is (CH₂CH₂O)ₓCH₂CH₂COOH, or (CH₂)_{y}COOH;
R^{7c} is H or C₁-C₆ alkyl; or
R^{7b} and R^{7c}, together with the nitrogen to which they are attached, combine to form a heterocycloalkyl group optionally substituted with C₁-C₆ alkyl which is optionally substituted with OH, COOH, or amino;
R^{R} is H, C₁-C₆ haloalkyl, COOH, C(═O)NH—R^{8a}, 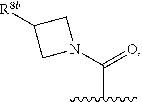
or C(═O)NH(CH₂)₂NHC(═O)—R^{8c};
R^{8a} is C₁-C₂₀ alkyl substituted with COOH or SO₃H, or C₁-C₆ alkoxy optionally substituted with amino;
R^{8b} is COOH or amino;
R^{8c} is (CH₂CH₂O)ₓCH₂CH₂COOH or (CH₂)_{y}COOH;
each x is independently an integer selected from 1-10;
each y is independently an integer selected from 1-20;
R^{d} is the residue of a bioactive molecule of the formula R^{d}—H;
wherein when R⁷ is H, then R⁸ is C₁-C₆ haloalkyl, COOH, C(═O)NH—R^{8a}, 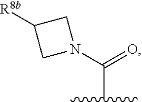
or C(═O)NH(CH₂)₂NHC(═O)—R^{8c}; and
wherein when R⁸ is H, then R⁷ is C₁-C₆ haloalkyl, OR^{7a}, 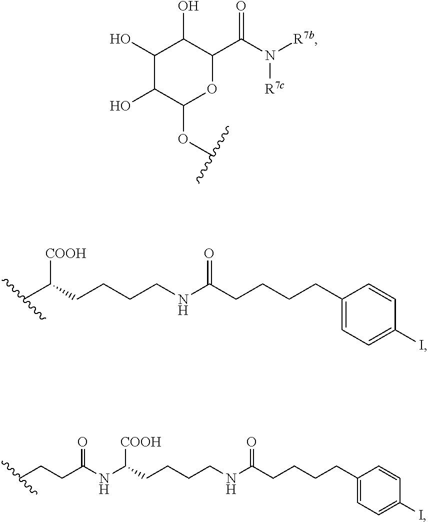
or C₁-C₆ alkyl optionally substituted with COOH.

56. The compound of claim 55, or a pharmaceutically acceptable salt thereof, wherein:
J is a bond or NR⁶;
n is an integer selected from 1-5;
R^{A} is 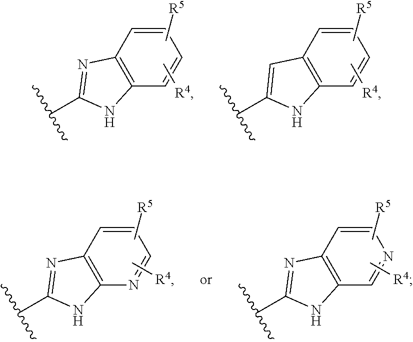
Y is N or CH;
Z is N or CR¹⁰;
R¹ is H;
R² is H or C₁-C₆ alkyl; or
R¹ and R², together with the N and C to which they are attached, combine to form a heterocycloalkyl;
R³ is C₁-C₆ alkyl or C₁-C₆ alkoxy;
R⁴ is H, halogen, CN, or C₁-C₆ alkoxy;
R⁵ is H, halogen, or CN;
R⁶ is H or C₁-C₆ alkyl;
R⁹ is H or C₁-C₆ alkyl;
R¹⁰ is H or halogen;
Spacer is a spacer group;
each A is independently selected from natural or unnatural amino acids, wherein any free amine of an amino acid is optionally independently substituted with CH₃;
w is an integer selected from 1-6;
R⁷ is H, C₁-C₆ alkyl, C₁-C₆ haloalkyl, OR⁷³, or 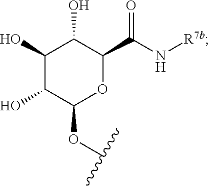
R^{7a} is H, or C₁-C₆ alkyl optionally substituted with amino, COOH, or OH;
R^{7b} is H, C₁-C₂₀ alkyl substituted with COOR^{7b1}, amino, or SO₃H, (CH₂)₂NHC(═O)—R^{7b2}, 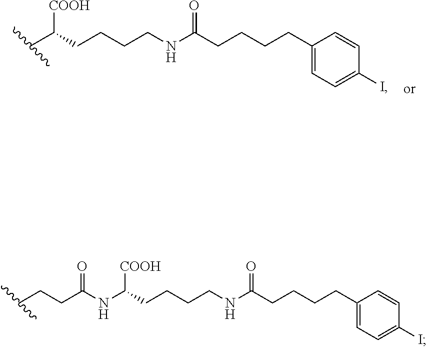
R^{7b1} is H or C₁-C₆ alkyl;
R^{7b2} is (CH₂CH₂O)ₓCH₂CH₂COOH, or (CH₂)_{y}COOH;
R⁸ is H, C₁-C₆ haloalkyl, COOH, C(═O)NH—R^{8a}, 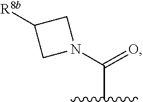
or C(═O)NH(CH₂)₂NHC(═O)—R^{8c};
R^{8a} is C₁-C₂₀ alkyl substituted with COOH or SO₃H, or C₁-C₆ alkoxy optionally substituted with amino;
R^{8b} is COOH or amino;
R^{8c} is (CH₂CH₂O)ₓCH₂CH₂COOH or (CH₂)_{y}COOH;
each x is independently an integer selected from 1-10;
each y is independently an integer selected from 1-20;
R^{d} is the residue of a bioactive molecule of the formula R^{d}—H;
wherein when R⁷ is H, then R⁸ is C₁-C₆ haloalkyl, COOH, C(═O)NH—R^{8a}, 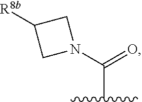
or C(═O)NH(CH₂)₂NHC(═O)—R^{8c}; and
wherein when R⁸ is H, then R⁷ is C₁-C₆ alkyl, C₁-C₆ haloalkyl, OR^{7a}, or

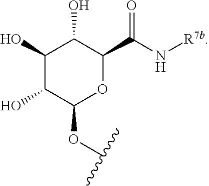

57. The compound of claim 55, or a pharmaceutically acceptable salt thereof, wherein R⁷ is

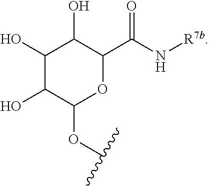

58. The compound of claim 55, or a pharmaceutically acceptable salt thereof, wherein R⁷ is OR^{7a} or

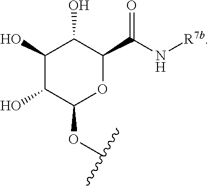

59. The compound of claim 55, or a pharmaceutically acceptable salt thereof, wherein 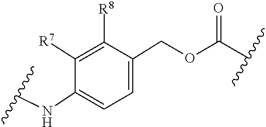
of Formula (I) is: 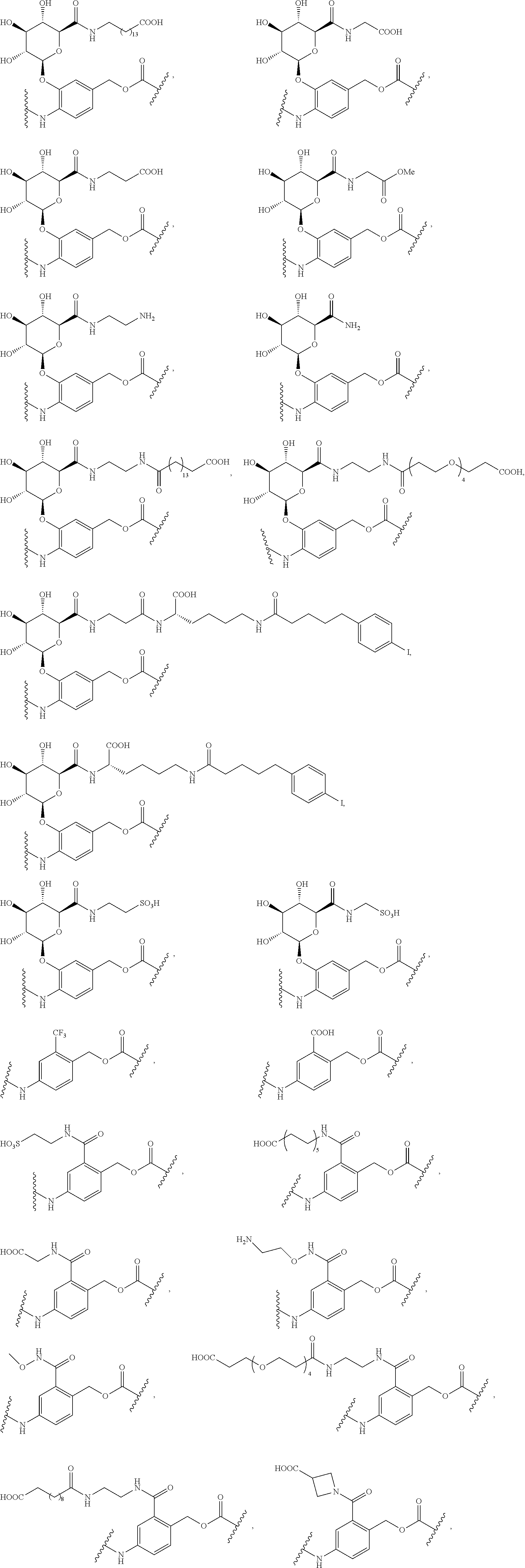 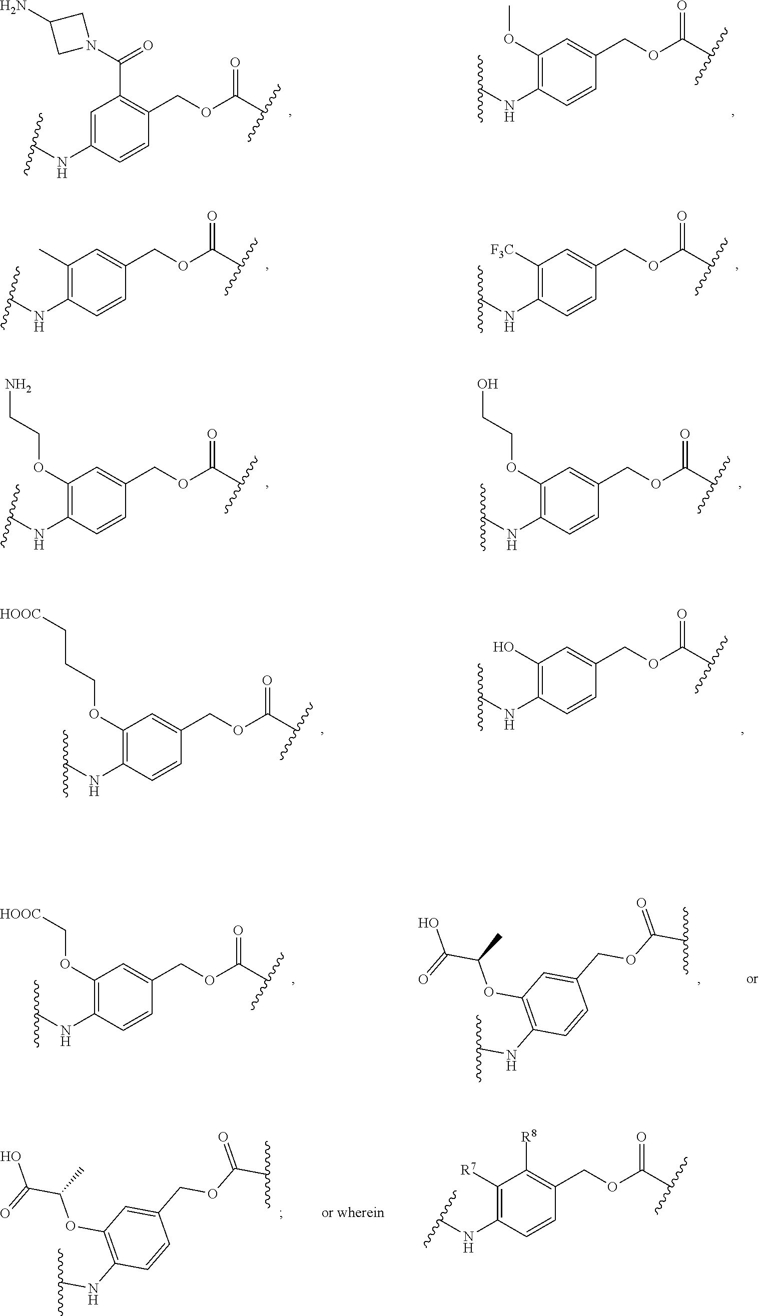
of Formula (I) is:

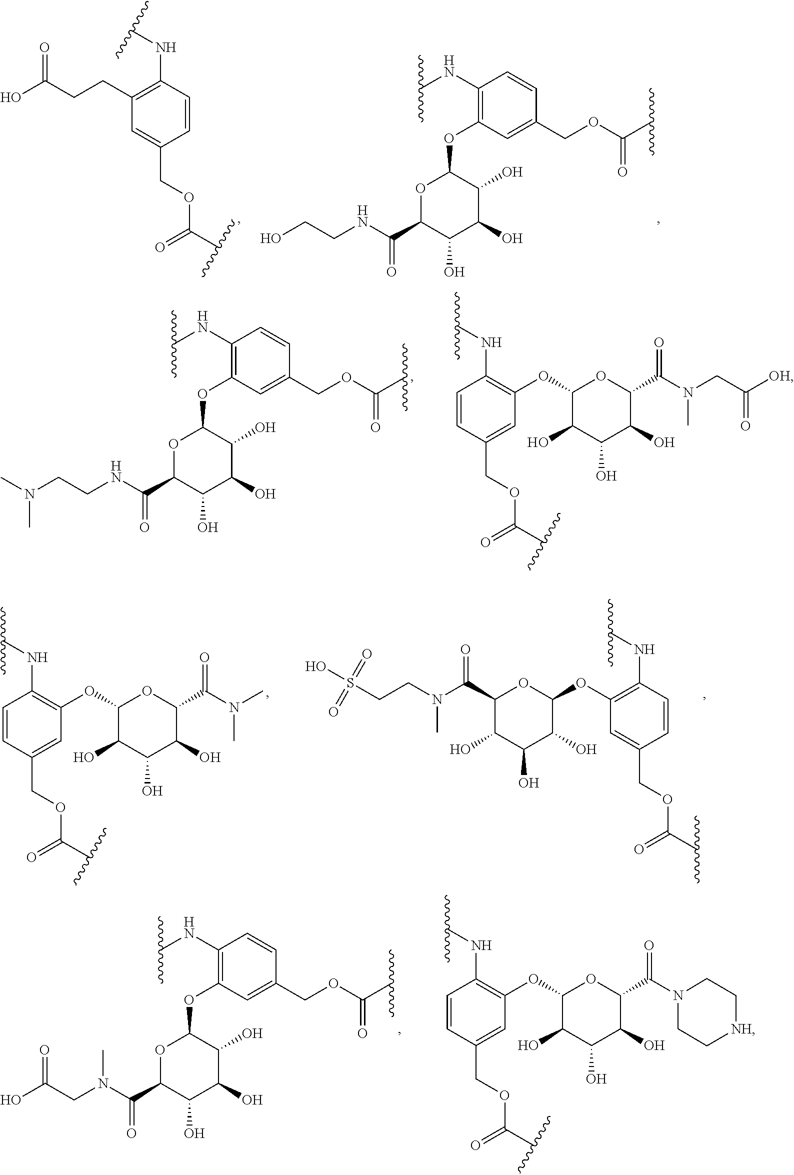

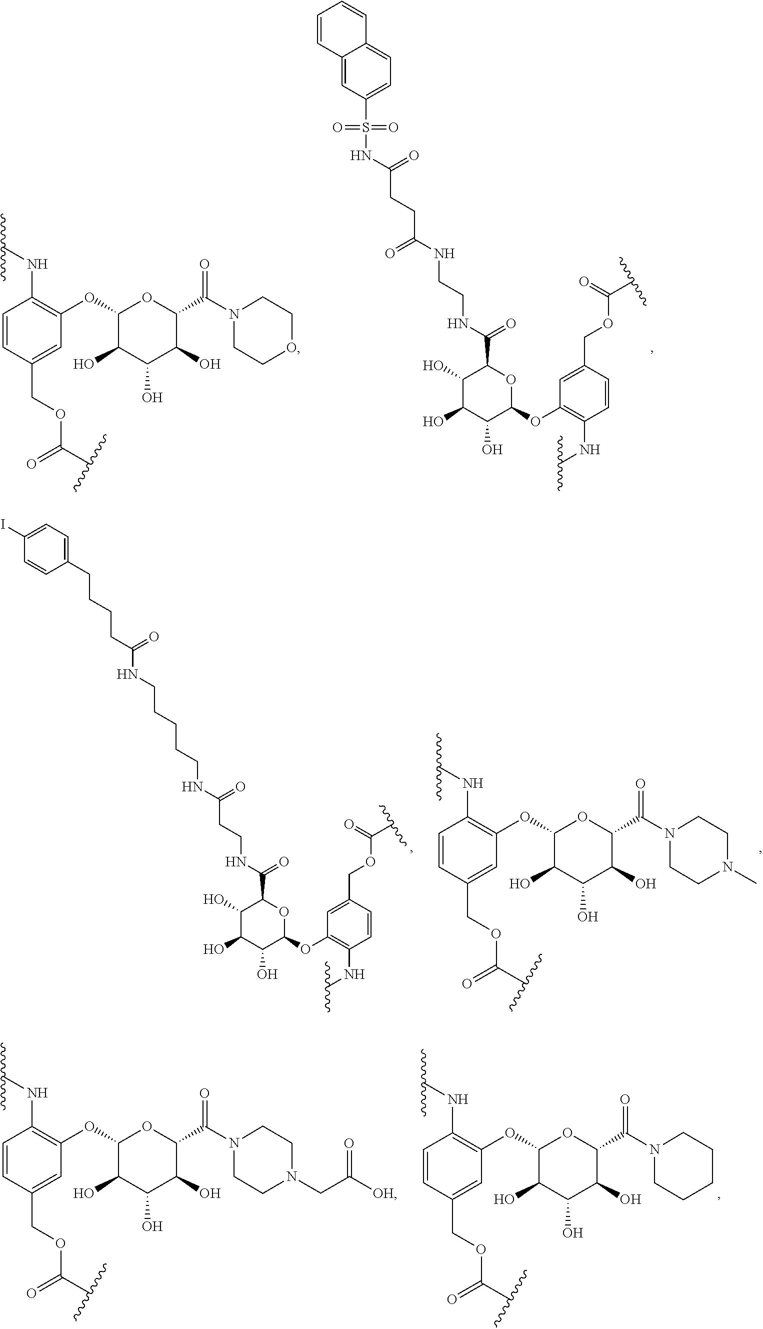

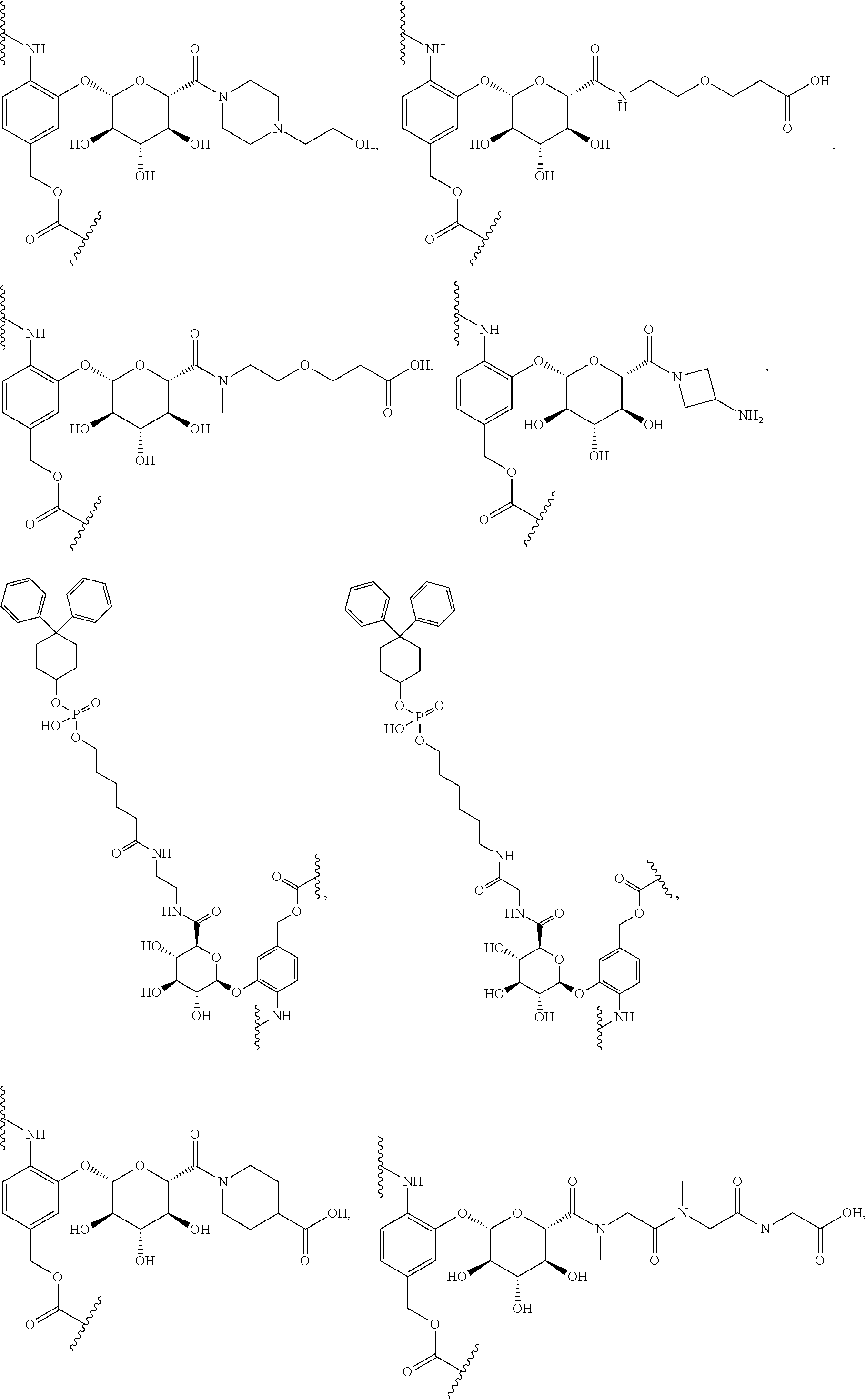

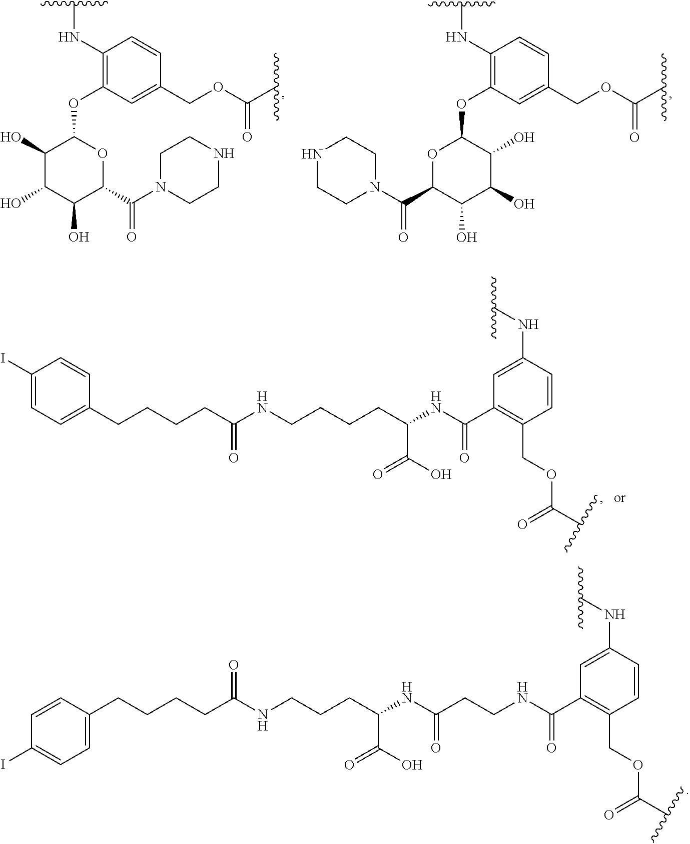 [SMILES: *Nc1ccc(COC(*)=O)cc1O[C@@H]1O[C@H](C(=O)N2CCNCC2)[C@@H](O)[C@H](O)[C@H]1O ; *Nc1ccc(COC(*)=O)cc1O[C@@H]1O[C@H](C(=O)N2CCNCC2)[C@@H](O)[C@H](O)[C@H]1O ; *Nc1ccc(COC(*)=O)c(C(=O)N[C@@H](CCCCNC(=O)CCCCc2ccc(I)cc2)C(=O)O)c1 ; *Nc1ccc(COC(*)=O)c(C(=O)NCCC(=O)N[C@@H](CCCNC(=O)CCCCc2ccc(I)cc2)C(=O)O)c1]

60. The compound of claim 55, or a pharmaceutically acceptable salt thereof, wherein 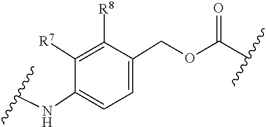
of Formula (I) is:

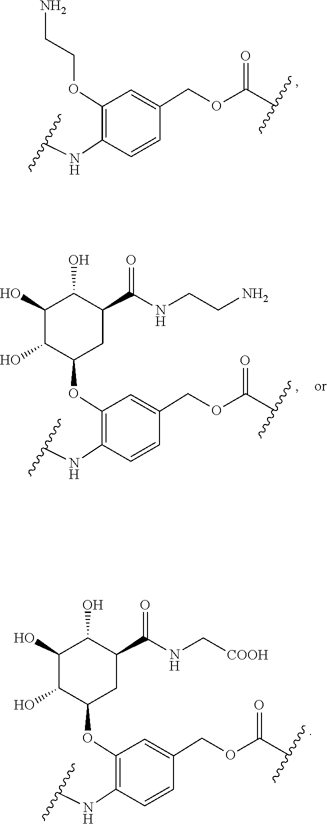 [SMILES: *Nc1ccc(COC(*)=O)cc1OCCN ; *Nc1ccc(COC(*)=O)cc1O[C@@H]1C[C@H](C(=O)NCCN)[C@@H](O)[C@H](O)[C@H]1O ; *Nc1ccc(COC(*)=O)cc1O[C@@H]1C[C@H](C(=O)NCC(=O)O)[C@@H](O)[C@H](O)[C@H]1O]

61. The compound of claim 55, or a pharmaceutically acceptable salt thereof, wherein each A is independently selected from natural or unnatural amino acids, wherein any free amine of an amino acid is optionally independently substituted with CH₃, wherein the natural or unnatural amino acids are selected from alanine (Ala), Ala(OH), Ala(SO₃H), 3-(1-piperidinyl)alanine, cyclohexylalanine, arginine (Arg), asparagine (Asn), aspartate (Asp), cysteine (Cys), glutamine (GIn), glutamatic acid (Glu), glycine (Gly), leucine (Leu), lysine(Lys), methionine(Met), phenylalanine (Phe), homophenylalanine, proline (Pro), serine (Ser), 3-homoserine, tyrosine (Tyr), Tyr(SO₃H), tryptophan (Trp), valine (Val), citrulline, p-alanine, β3-homoserine, β3-homolysine, and β3-homoglutamic acid.

62. The compound of claim 55, or a pharmaceutically acceptable salt thereof, wherein each A is independently selected from natural or unnatural amino acids, wherein any free amine of an amino acid is optionally independently substituted with CH₃, wherein the natural or unnatural amino acids are selected from alanine (Ala), Ala(OH), Ala(SO₃H), glutamine (Gin), glutamatic acid (Glu), glycine (Gly), leucine (Leu), tryptophan (Trp), valine (Val), serine (Ser), and citrulline.

63. The compound of claim 55, or a pharmaceutically acceptable salt thereof, wherein w is an integer selected from 2 or 3.

64. The compound of claim 55, or a pharmaceutically acceptable salt thereof, wherein (A), is valine-citrulline, valine-alanine, alanine(SO₃H)-valine-alanine, glutamic acid-valine-citrulline, glycine-valine-citrulline, glutamine-valine-citrulline, serine-valine-citrulline, alanine(SO₃H)-valine-citrulline, or tryptophane-valine-citulline.

65. The compound of claim 55, or a pharmaceutically acceptable salt thereof, wherein (A), is valine-citrulline, valine-alanine, or glutamic acid-valine-citrulline.

66. The compound of claim 55, or a pharmaceutically acceptable salt thereof, wherein 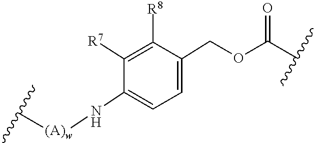
of Formula (I) is:

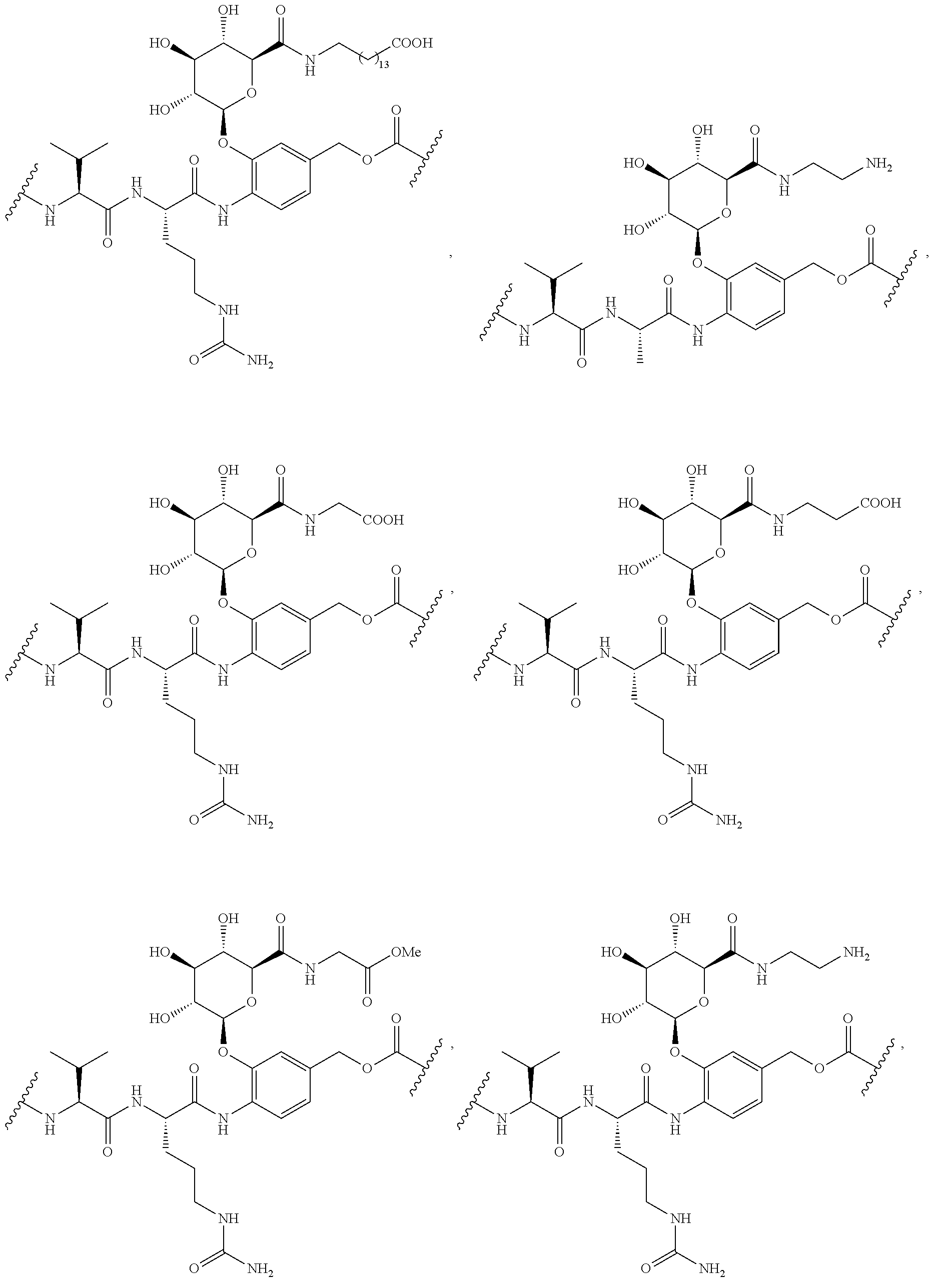

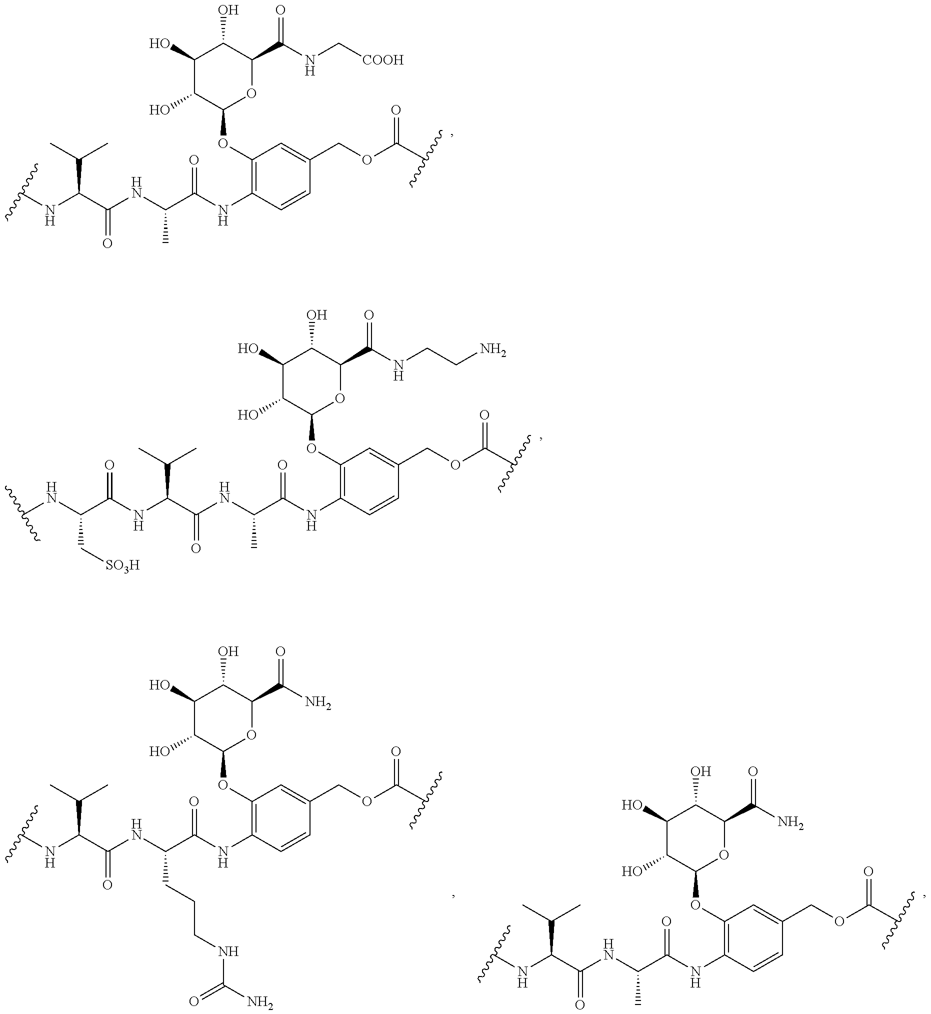 [SMILES: *N[C@H](C(=O)N[C@@H](C)C(=O)Nc1ccc(COC(*)=O)cc1O[C@@H]1O[C@H](C(=O)NCC(=O)O)[C@@H](O)[C@H](O)[C@H]1O)C(C)C ; *N[C@@H](CS(=O)(=O)O)C(=O)N[C@H](C(=O)N[C@@H](C)C(=O)Nc1ccc(COC(*)=O)cc1O[C@@H]1O[C@H](C(=O)NCCN)[C@@H](O)[C@H](O)[C@H]1O)C(C)C ; *N[C@H](C(=O)N[C@@H](CCCNC(N)=O)C(=O)Nc1ccc(COC(*)=O)cc1O[C@@H]1O[C@H](C(N)=O)[C@@H](O)[C@H](O)[C@H]1O)C(C)C ; *N[C@H](C(=O)N[C@@H](C)C(=O)Nc1ccc(COC(*)=O)cc1O[C@@H]1O[C@H](C(N)=O)[C@@H](O)[C@H](O)[C@H]1O)C(C)C]

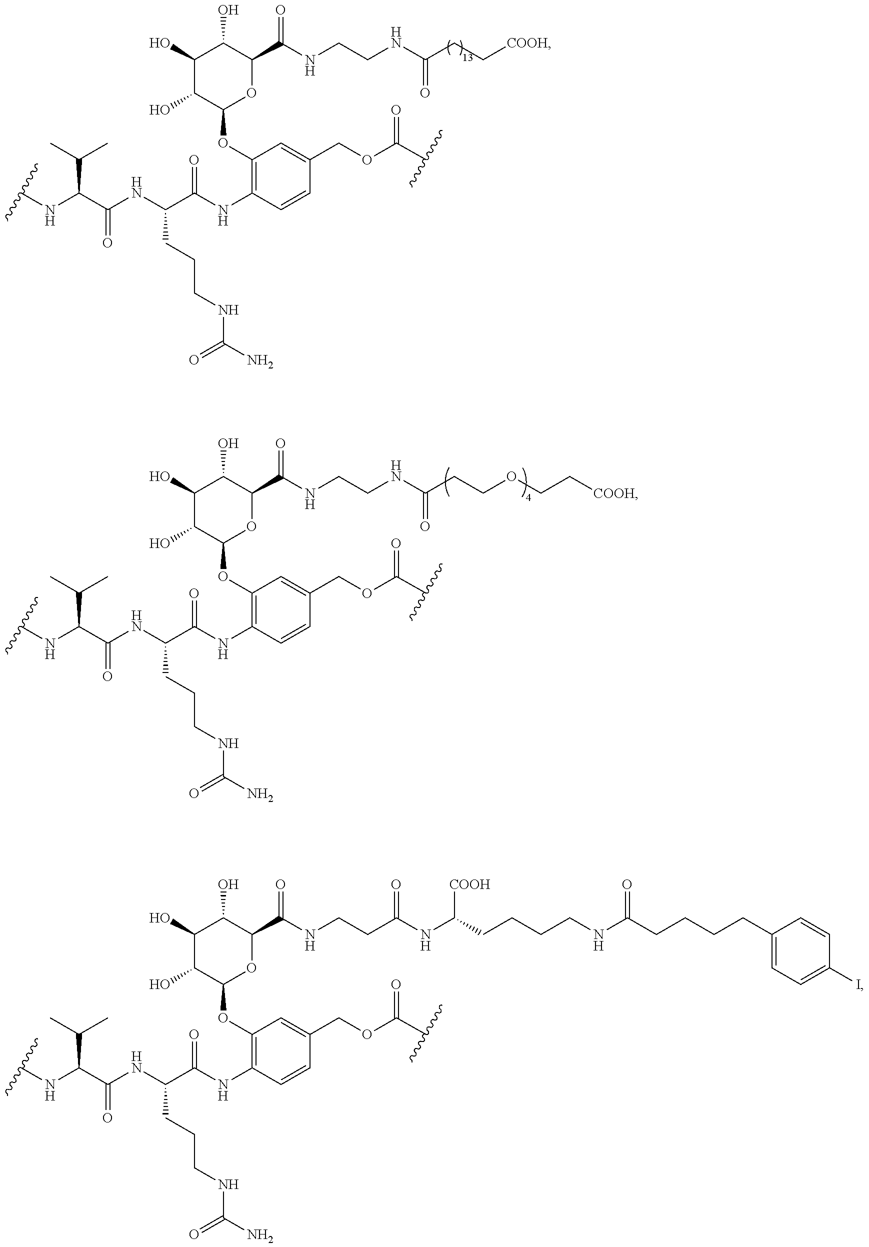

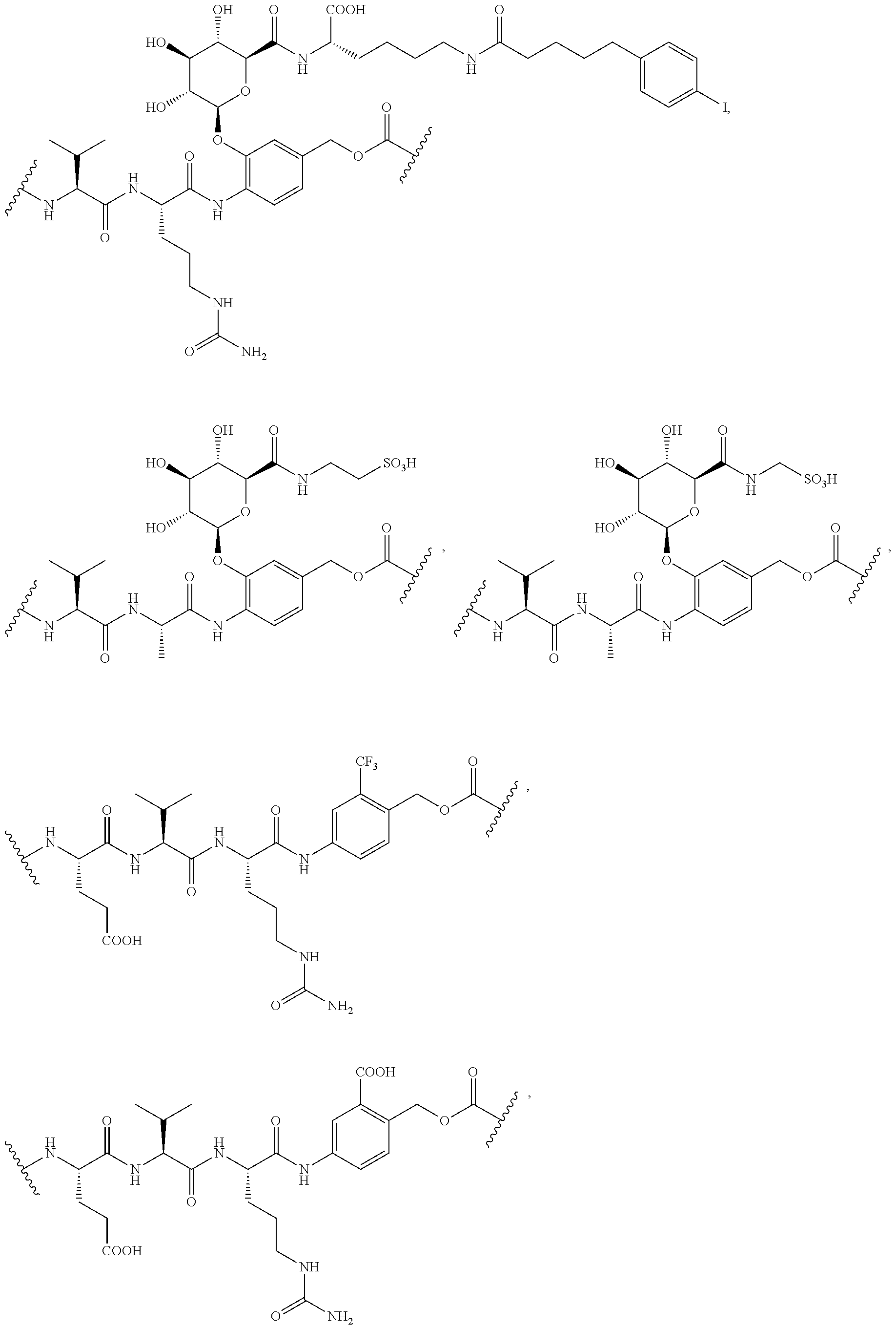

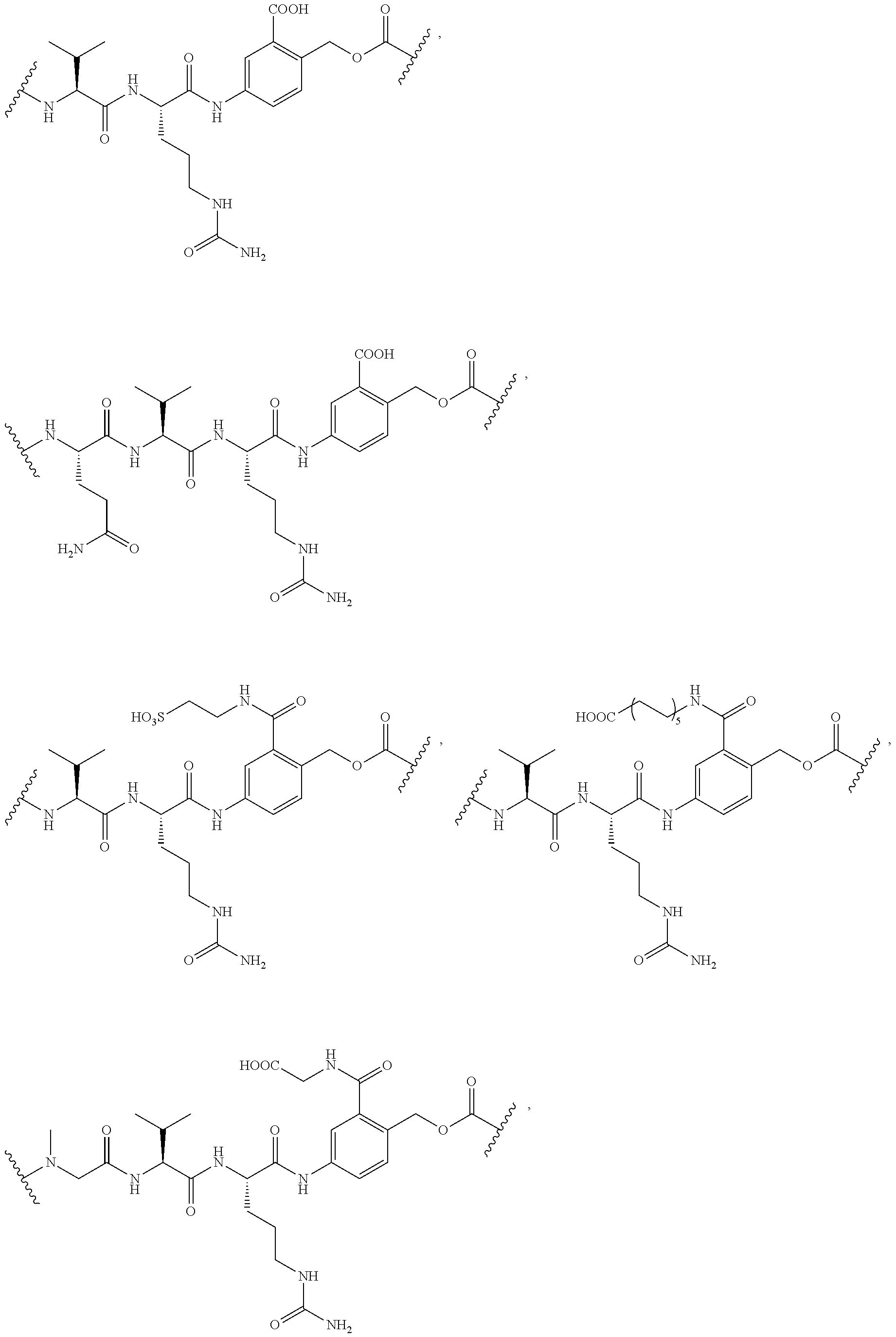

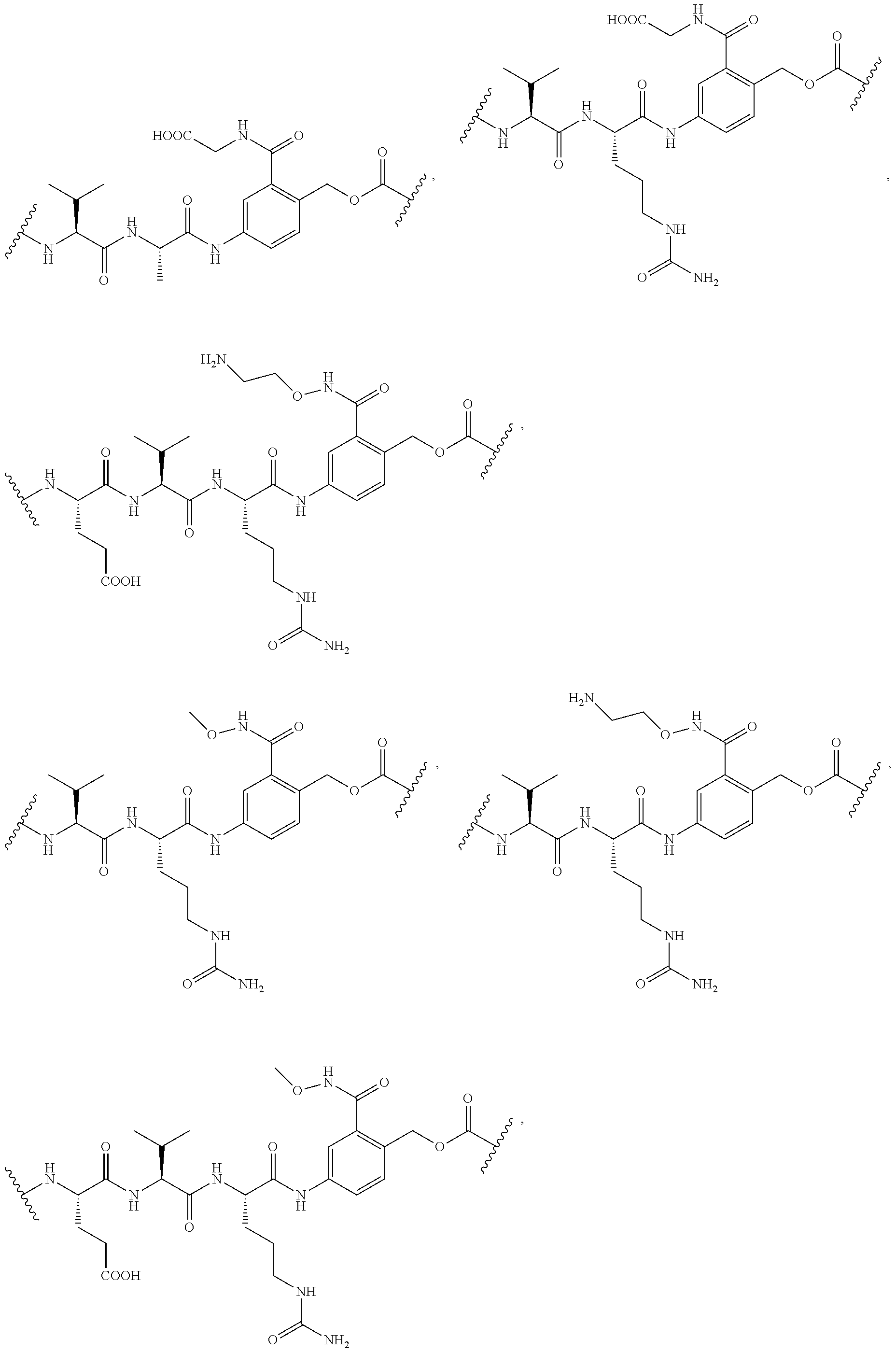 [SMILES: *N[C@H](C(=O)N[C@@H](C)C(=O)Nc1ccc(COC(*)=O)c(C(=O)NCC(=O)O)c1)C(C)C ; *N[C@H](C(=O)N[C@@H](CCCNC(N)=O)C(=O)Nc1ccc(COC(*)=O)c(C(=O)NCC(=O)O)c1)C(C)C ; *N[C@@H](CCC(=O)O)C(=O)N[C@H](C(=O)N[C@@H](CCCNC(N)=O)C(=O)Nc1ccc(COC(*)=O)c(C(=O)NOCCN)c1)C(C)C ; *N[C@H](C(=O)N[C@@H](CCCNC(N)=O)C(=O)Nc1ccc(COC(*)=O)c(C(=O)NOC)c1)C(C)C ; *N[C@H](C(=O)N[C@@H](CCCNC(N)=O)C(=O)Nc1ccc(COC(*)=O)c(C(=O)NOCCN)c1)C(C)C ; *N[C@@H](CCC(=O)O)C(=O)N[C@H](C(=O)N[C@@H](CCCNC(N)=O)C(=O)Nc1ccc(COC(*)=O)c(C(=O)NOC)c1)C(C)C]

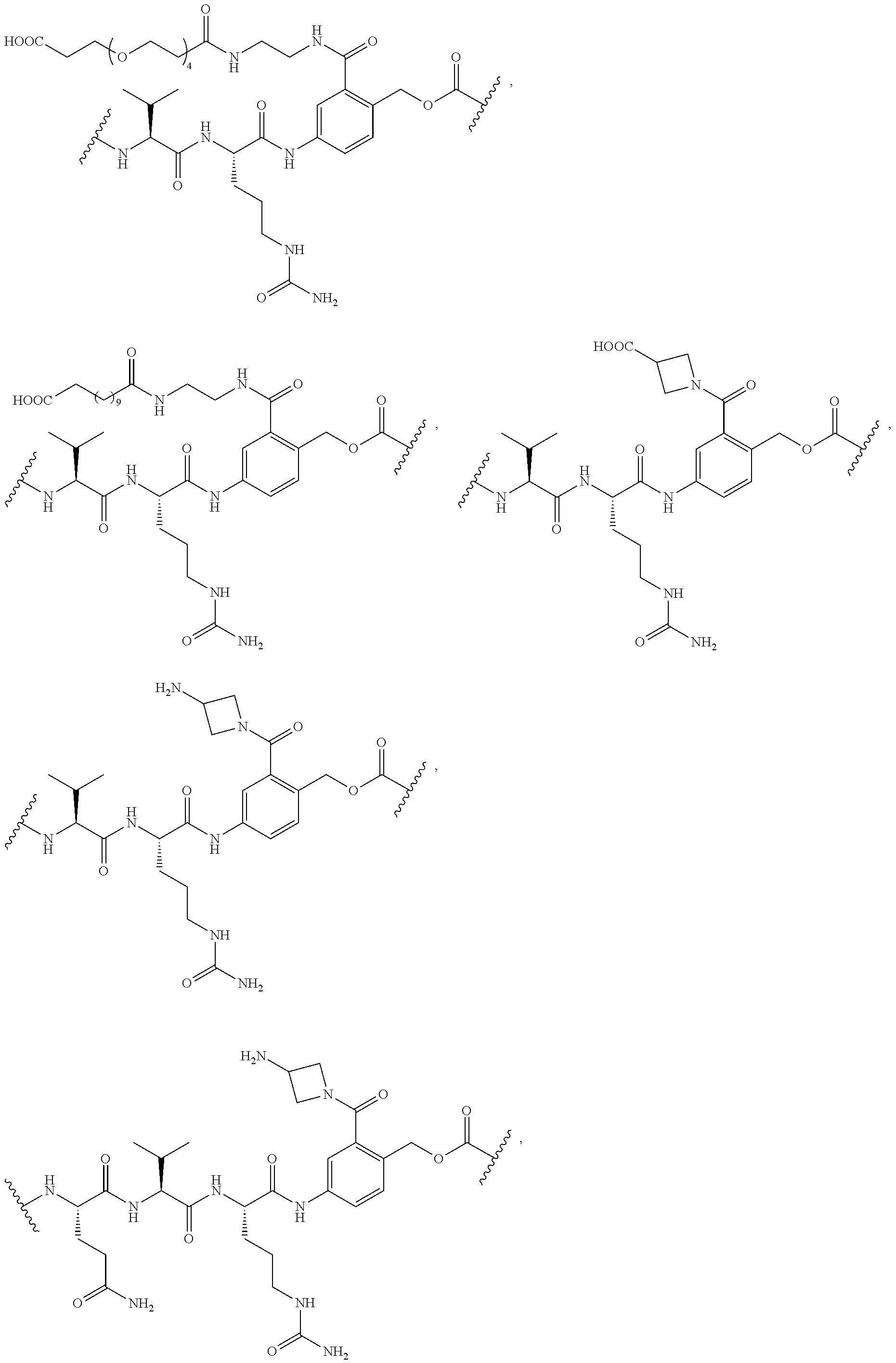

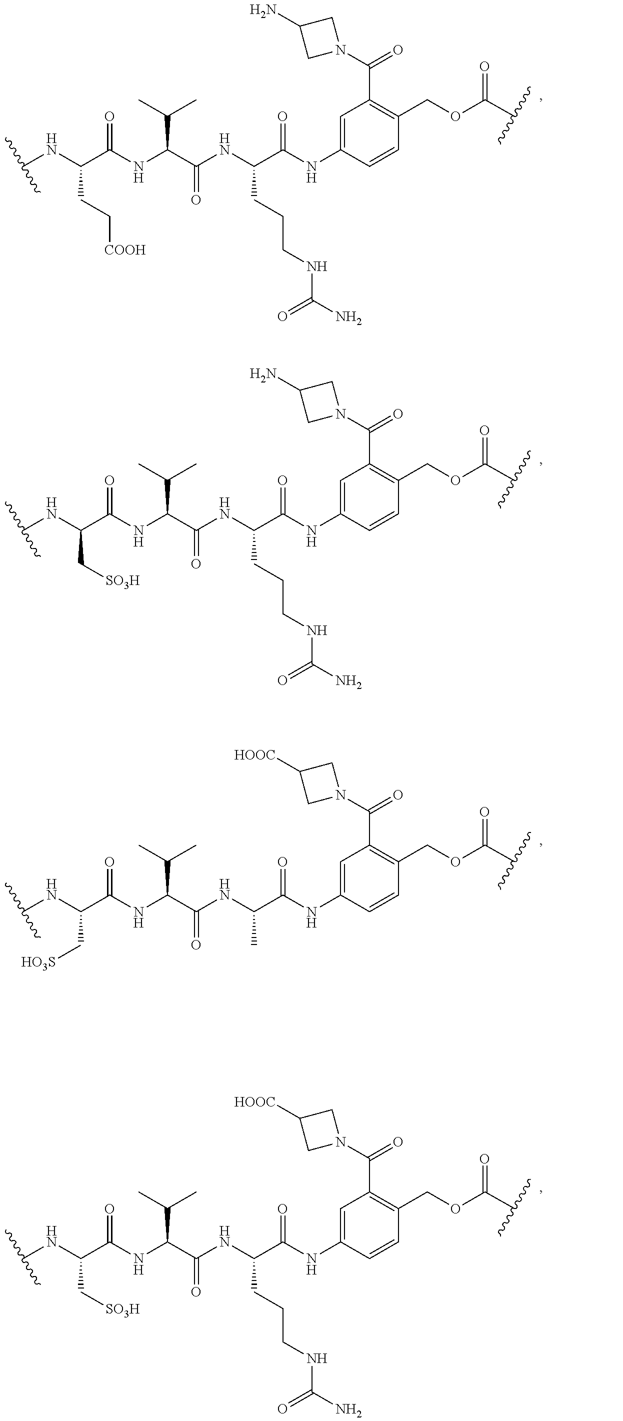 [SMILES: *N[C@@H](CCC(=O)O)C(=O)N[C@H](C(=O)N[C@@H](CCCNC(N)=O)C(=O)Nc1ccc(COC(*)=O)c(C(=O)N2CC(N)C2)c1)C(C)C ; *N[C@H](CS(=O)(=O)O)C(=O)N[C@H](C(=O)N[C@@H](CCCNC(N)=O)C(=O)Nc1ccc(COC(*)=O)c(C(=O)N2CC(N)C2)c1)C(C)C ; *N[C@@H](CS(=O)(=O)O)C(=O)N[C@H](C(=O)N[C@@H](C)C(=O)Nc1ccc(COC(*)=O)c(C(=O)N2CC(C(=O)O)C2)c1)C(C)C ; *N[C@@H](CS(=O)(=O)O)C(=O)N[C@H](C(=O)N[C@@H](CCCNC(N)=O)C(=O)Nc1ccc(COC(*)=O)c(C(=O)N2CC(C(=O)O)C2)c1)C(C)C]

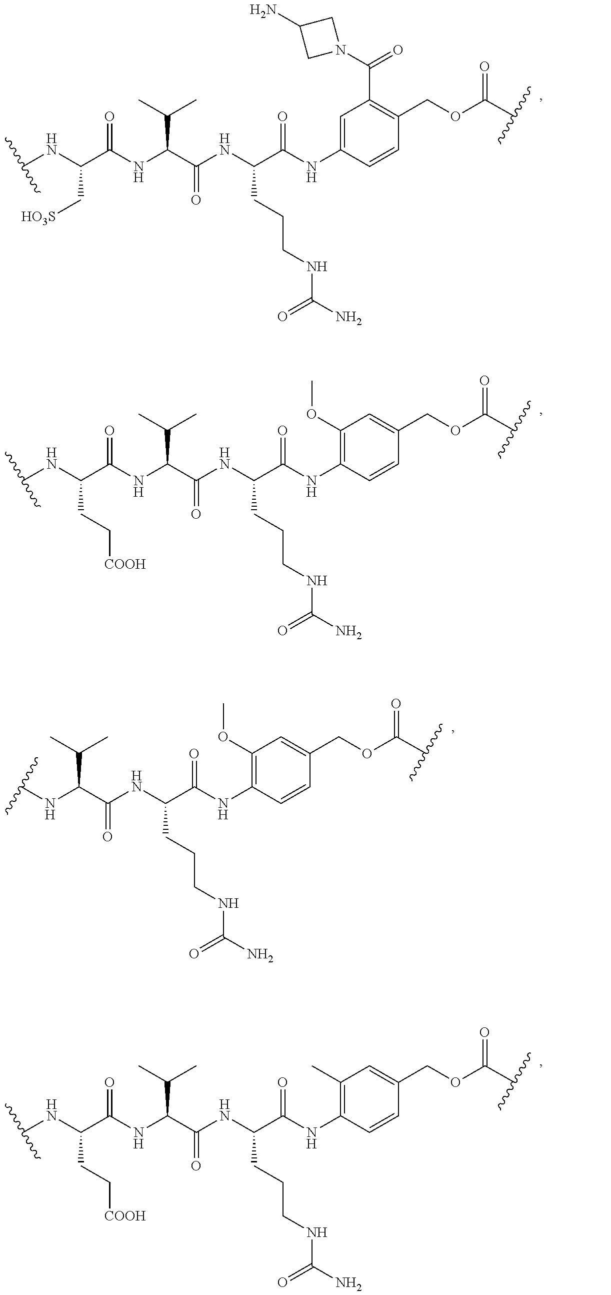 [SMILES: *N[C@@H](CS(=O)(=O)O)C(=O)N[C@H](C(=O)N[C@@H](CCCNC(N)=O)C(=O)Nc1ccc(COC(*)=O)c(C(=O)N2CC(N)C2)c1)C(C)C ; *N[C@@H](CCC(=O)O)C(=O)N[C@H](C(=O)N[C@@H](CCCNC(N)=O)C(=O)Nc1ccc(COC(*)=O)cc1OC)C(C)C ; *N[C@H](C(=O)N[C@@H](CCCNC(N)=O)C(=O)Nc1ccc(COC(*)=O)cc1OC)C(C)C ; *N[C@@H](CCC(=O)O)C(=O)N[C@H](C(=O)N[C@@H](CCCNC(N)=O)C(=O)Nc1ccc(COC(*)=O)cc1C)C(C)C]

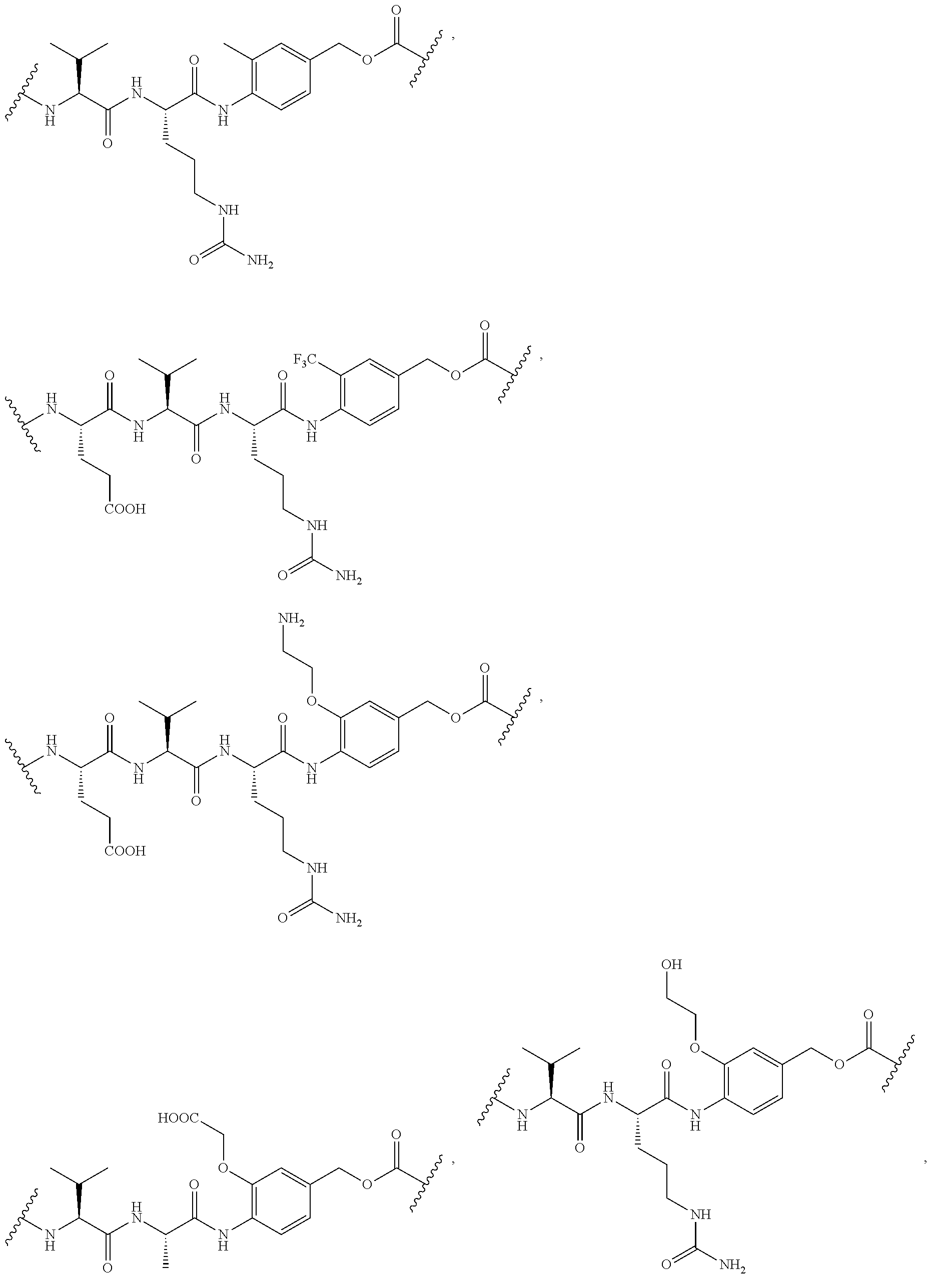

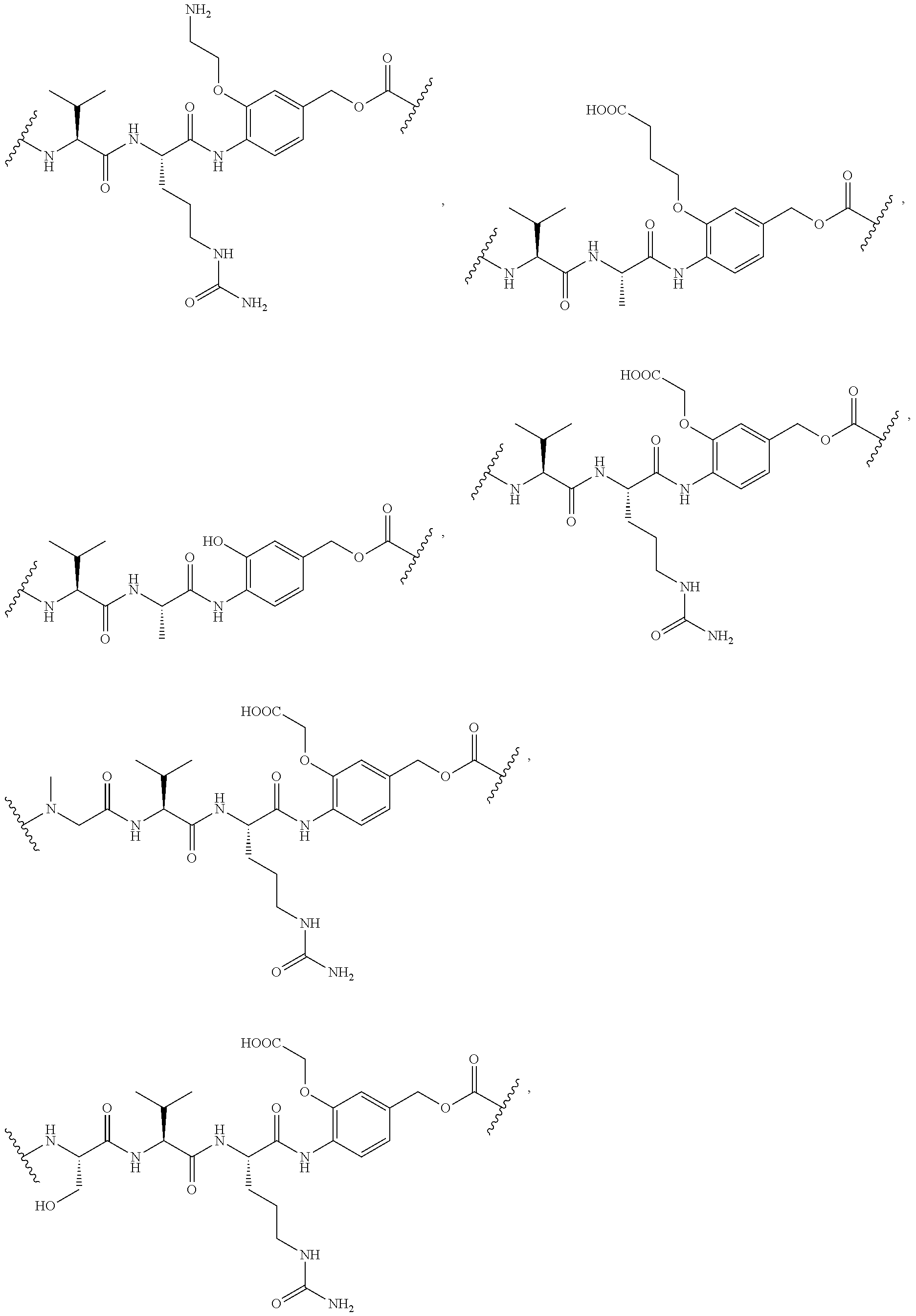 [SMILES: *N[C@H](C(=O)N[C@@H](CCCNC(N)=O)C(=O)Nc1ccc(COC(*)=O)cc1OCCN)C(C)C ; *N[C@H](C(=O)N[C@@H](C)C(=O)Nc1ccc(COC(*)=O)cc1OCCCC(=O)O)C(C)C ; *N[C@H](C(=O)N[C@@H](C)C(=O)Nc1ccc(COC(*)=O)cc1O)C(C)C ; *N[C@H](C(=O)N[C@@H](CCCNC(N)=O)C(=O)Nc1ccc(COC(*)=O)cc1OCC(=O)O)C(C)C ; *C(=O)OCc1ccc(NC(=O)[C@H](CCCNC(N)=O)NC(=O)[C@@H](NC(=O)CN(*)C)C(C)C)c(OCC(=O)O)c1 ; *N[C@@H](CO)C(=O)N[C@H](C(=O)N[C@@H](CCCNC(N)=O)C(=O)Nc1ccc(COC(*)=O)cc1OCC(=O)O)C(C)C]

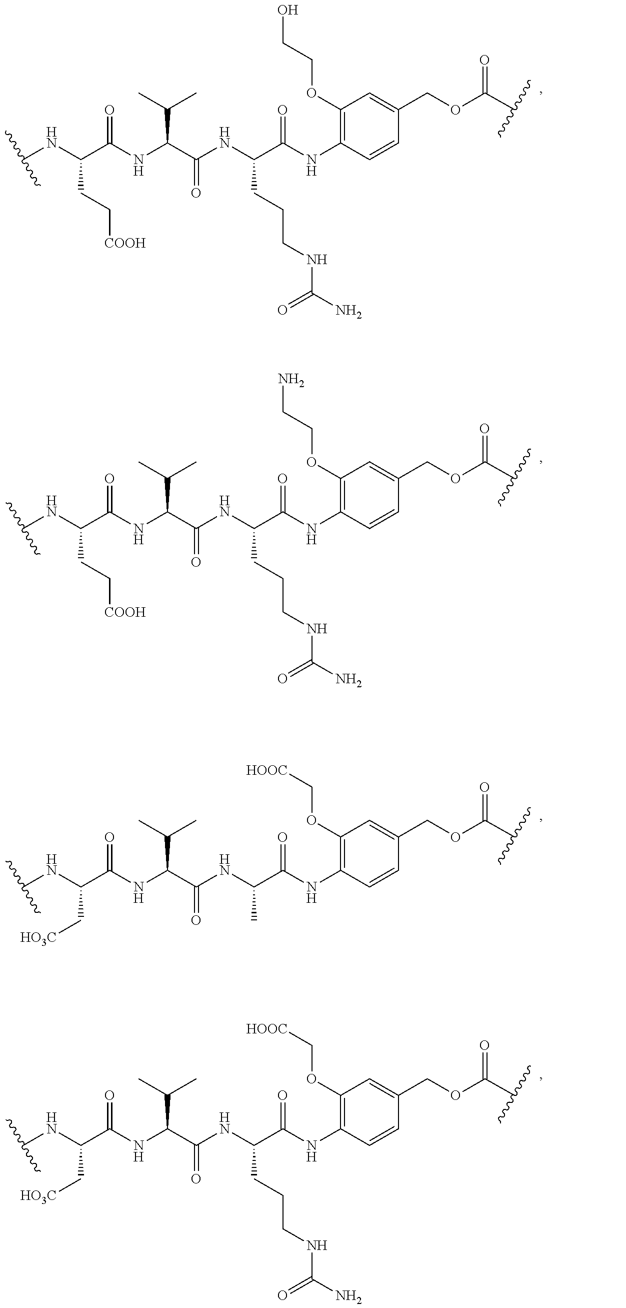

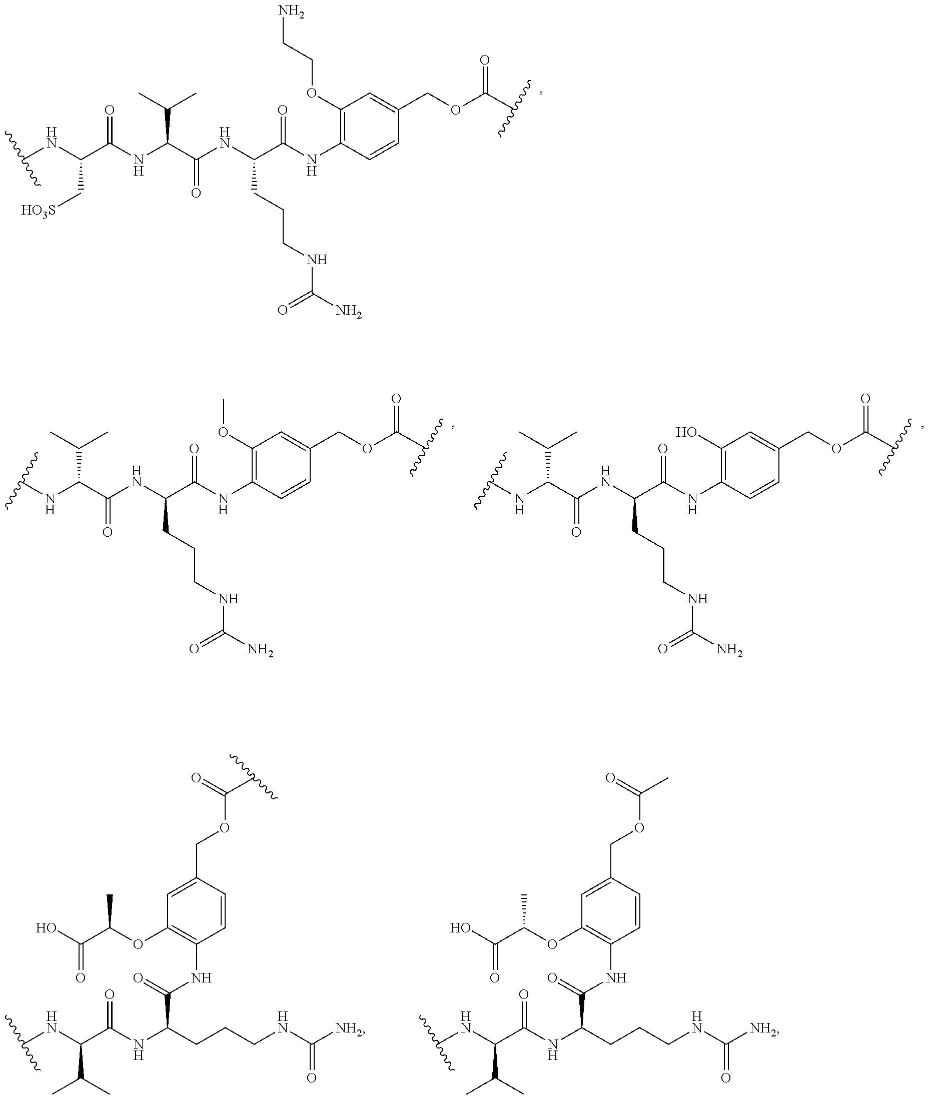 [SMILES: *N[C@@H](CS(=O)(=O)O)C(=O)N[C@H](C(=O)N[C@@H](CCCNC(N)=O)C(=O)Nc1ccc(COC(*)=O)cc1OCCN)C(C)C ; *N[C@@H](C(=O)N[C@H](CCCNC(N)=O)C(=O)Nc1ccc(COC(*)=O)cc1OC)C(C)C ; *N[C@@H](C(=O)N[C@H](CCCNC(N)=O)C(=O)Nc1ccc(COC(*)=O)cc1O)C(C)C ; *N[C@@H](C(=O)N[C@H](CCCNC(N)=O)C(=O)Nc1ccc(COC(*)=O)cc1O[C@H](C)C(=O)O)C(C)C ; *N[C@@H](C(=O)N[C@H](CCCNC(N)=O)C(=O)Nc1ccc(COC(C)=O)cc1O[C@@H](C)C(=O)O)C(C)C]

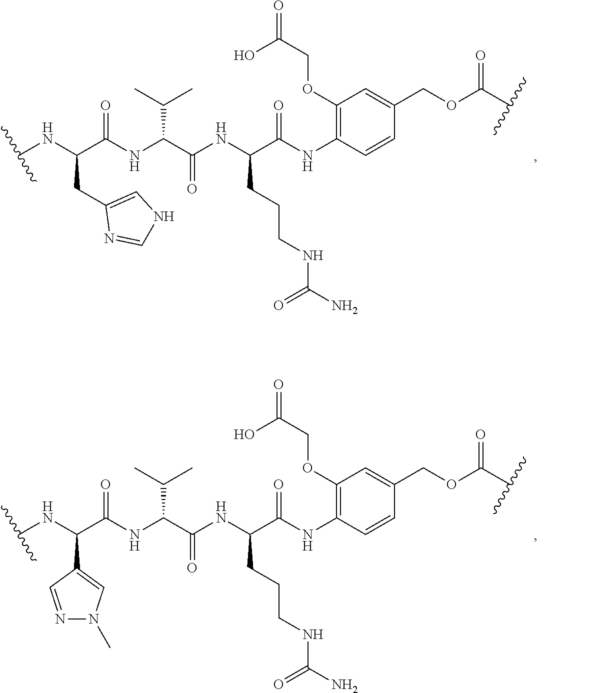

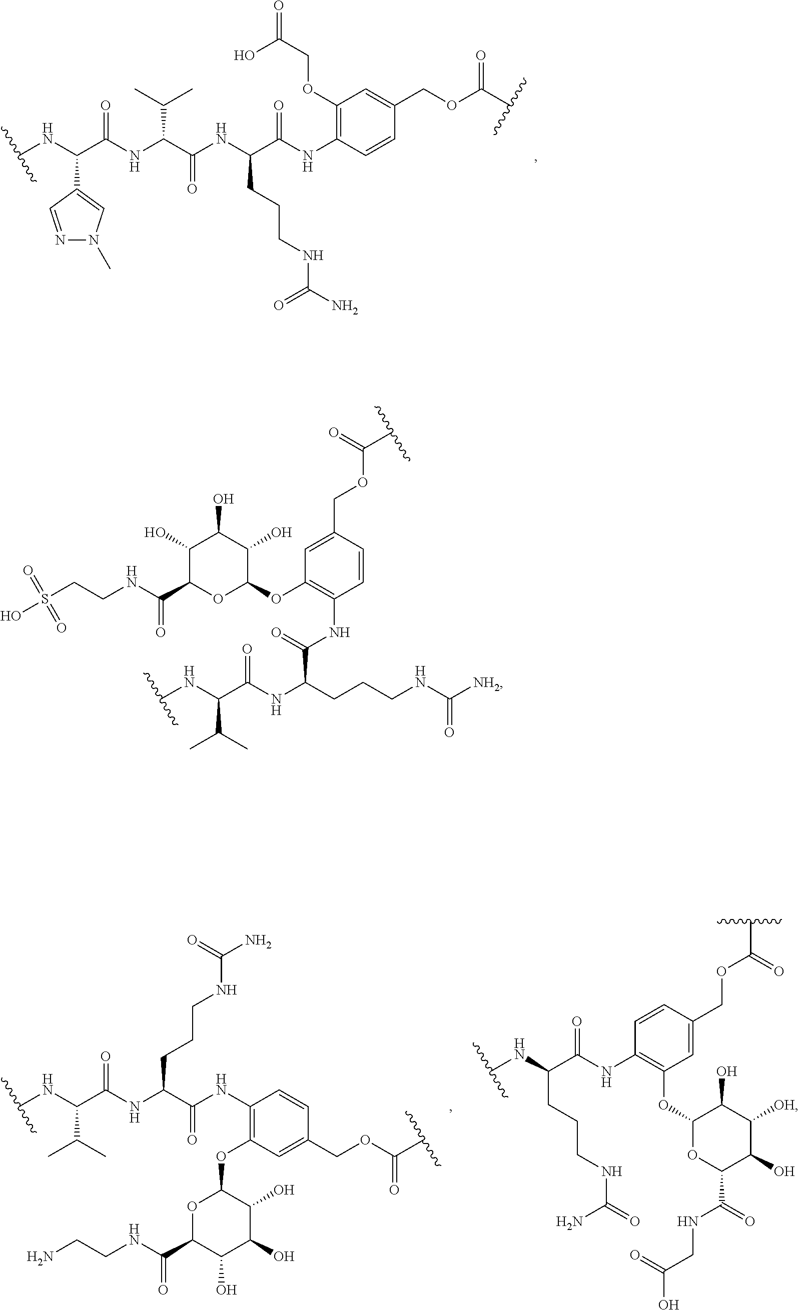 [SMILES: *N[C@H](C(=O)N[C@@H](C(=O)N[C@H](CCCNC(N)=O)C(=O)Nc1ccc(COC(*)=O)cc1OCC(=O)O)C(C)C)c1cnn(C)c1 ; *N[C@@H](C(=O)N[C@H](CCCNC(N)=O)C(=O)Nc1ccc(COC(*)=O)cc1O[C@H]1O[C@@H](C(=O)NCCS(=O)(=O)O)[C@H](O)[C@@H](O)[C@@H]1O)C(C)C ; *N[C@H](C(=O)N[C@@H](CCCNC(N)=O)C(=O)Nc1ccc(COC(*)=O)cc1O[C@@H]1O[C@H](C(=O)NCCN)[C@@H](O)[C@H](O)[C@H]1O)C(C)C ; *N[C@H](CCCNC(N)=O)C(=O)Nc1ccc(COC(*)=O)cc1O[C@H]1O[C@@H](C(=O)NCC(=O)O)[C@H](O)[C@@H](O)[C@@H]1O]

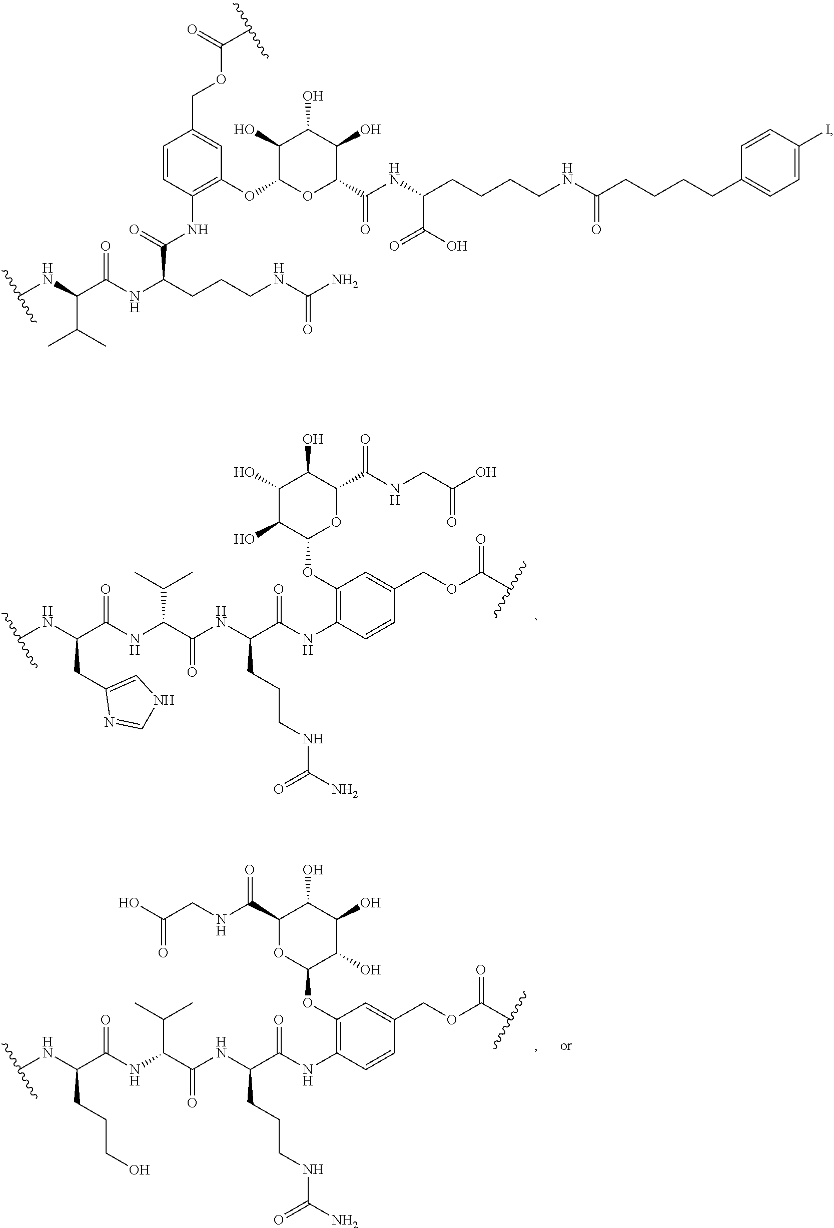 [SMILES: *N[C@@H](C(=O)N[C@H](CCCNC(N)=O)C(=O)Nc1ccc(COC(*)=O)cc1O[C@H]1O[C@@H](C(=O)N[C@H](CCCCNC(=O)CCCCc2ccc(I)cc2)C(=O)O)[C@H](O)[C@@H](O)[C@@H]1O)C(C)C ; *N[C@H](Cc1c[nH]cn1)C(=O)N[C@@H](C(=O)N[C@H](CCCNC(N)=O)C(=O)Nc1ccc(COC(*)=O)cc1O[C@H]1O[C@@H](C(=O)NCC(=O)O)[C@H](O)[C@@H](O)[C@@H]1O)C(C)C ; *N[C@H](CCCO)C(=O)N[C@@H](C(=O)N[C@H](CCCNC(N)=O)C(=O)Nc1ccc(COC(*)=O)cc1O[C@H]1O[C@@H](C(=O)NCC(=O)O)[C@H](O)[C@@H](O)[C@@H]1O)C(C)C]

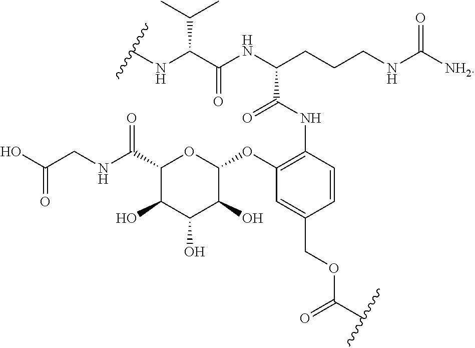 [SMILES: *N[C@@H](C(=O)N[C@H](CCCNC(N)=O)C(=O)Nc1ccc(COC(*)=O)cc1O[C@H]1O[C@@H](C(=O)NCC(=O)O)[C@H](O)[C@@H](O)[C@@H]1O)C(C)C]

67. The compound of claim 55, or a pharmaceutically acceptable salt thereof, wherein 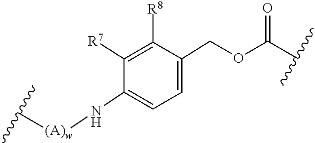
of Formula (I) is:

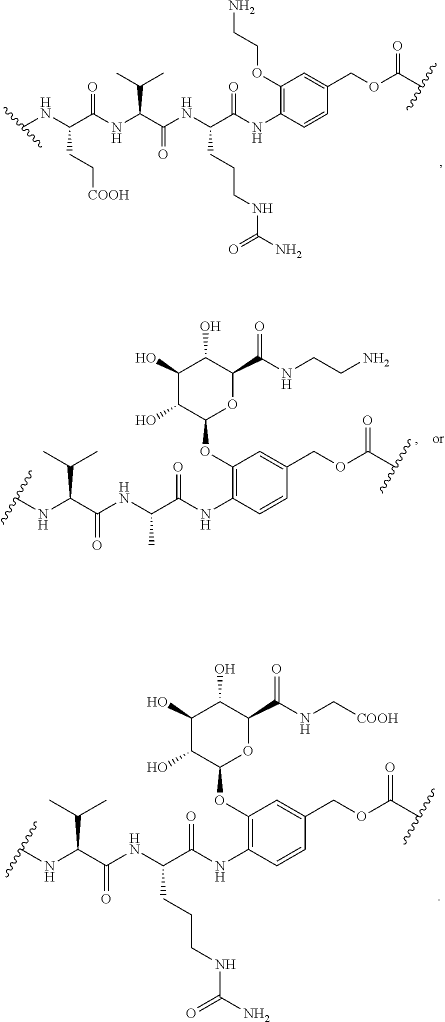 [SMILES: *N[C@@H](CCC(=O)O)C(=O)N[C@H](C(=O)N[C@@H](CCCNC(N)=O)C(=O)Nc1ccc(COC(*)=O)cc1OCCN)C(C)C ; *N[C@H](C(=O)N[C@@H](C)C(=O)Nc1ccc(COC(*)=O)cc1O[C@@H]1O[C@H](C(=O)NCCN)[C@@H](O)[C@H](O)[C@H]1O)C(C)C ; *N[C@H](C(=O)N[C@@H](CCCNC(N)=O)C(=O)Nc1ccc(COC(*)=O)cc1O[C@@H]1O[C@H](C(=O)NCC(=O)O)[C@@H](O)[C@H](O)[C@H]1O)C(C)C]

68. The compound of claim 55, or a pharmaceutically acceptable salt thereof, wherein Spacer is

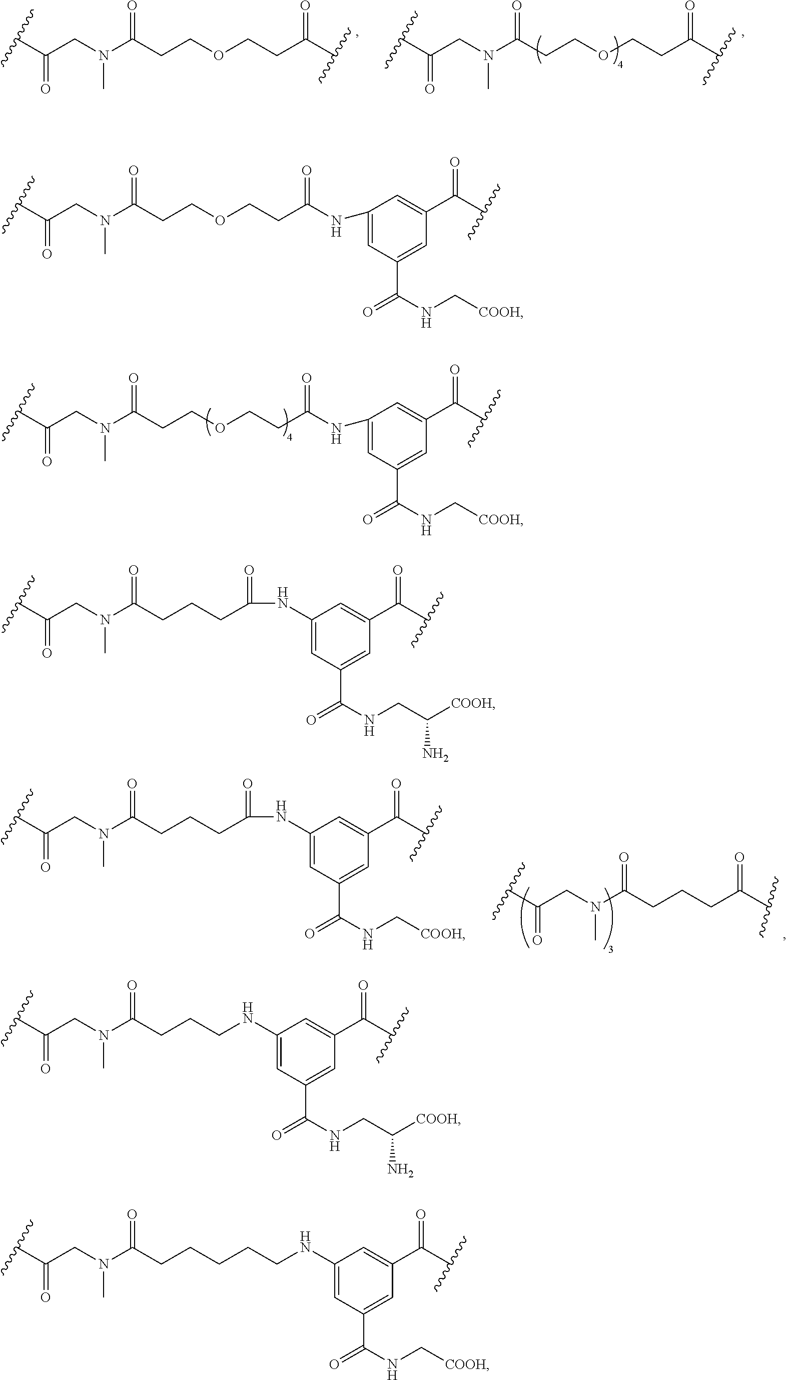

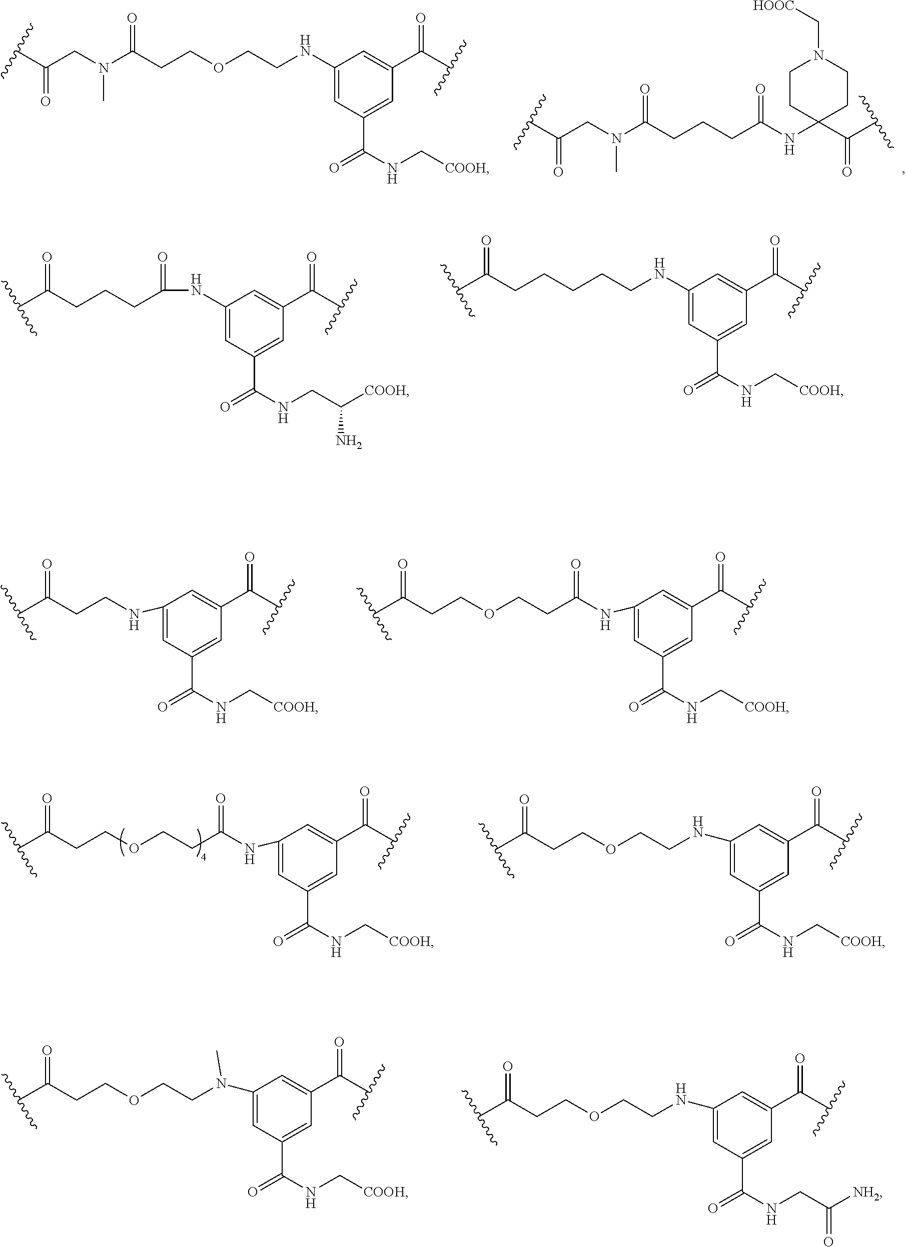

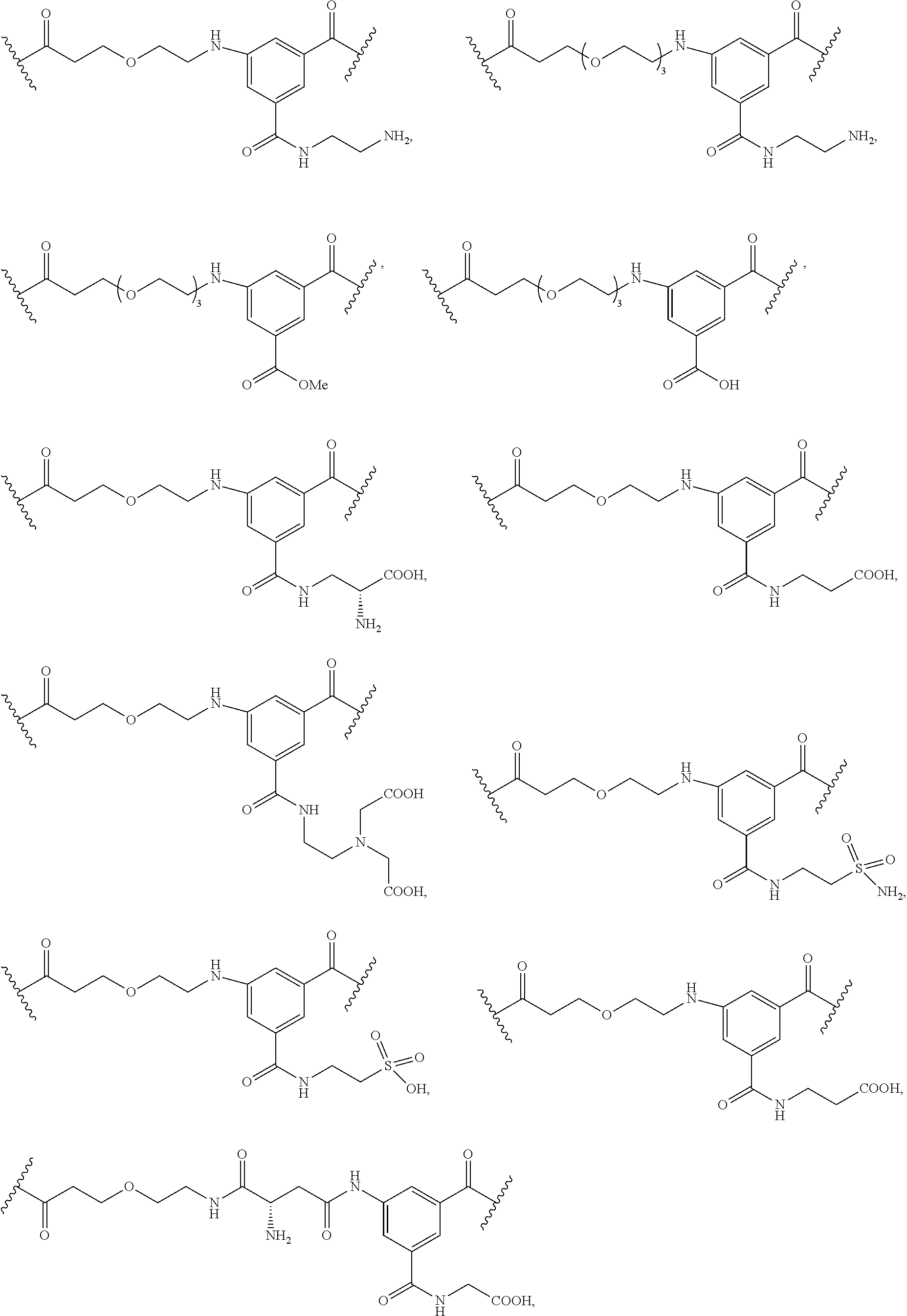

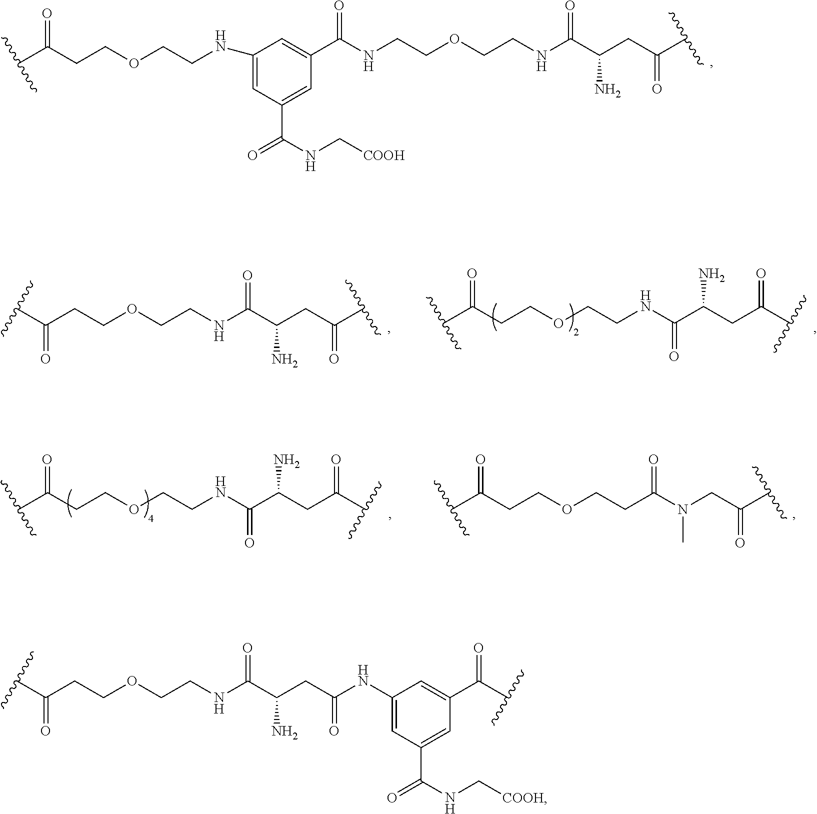

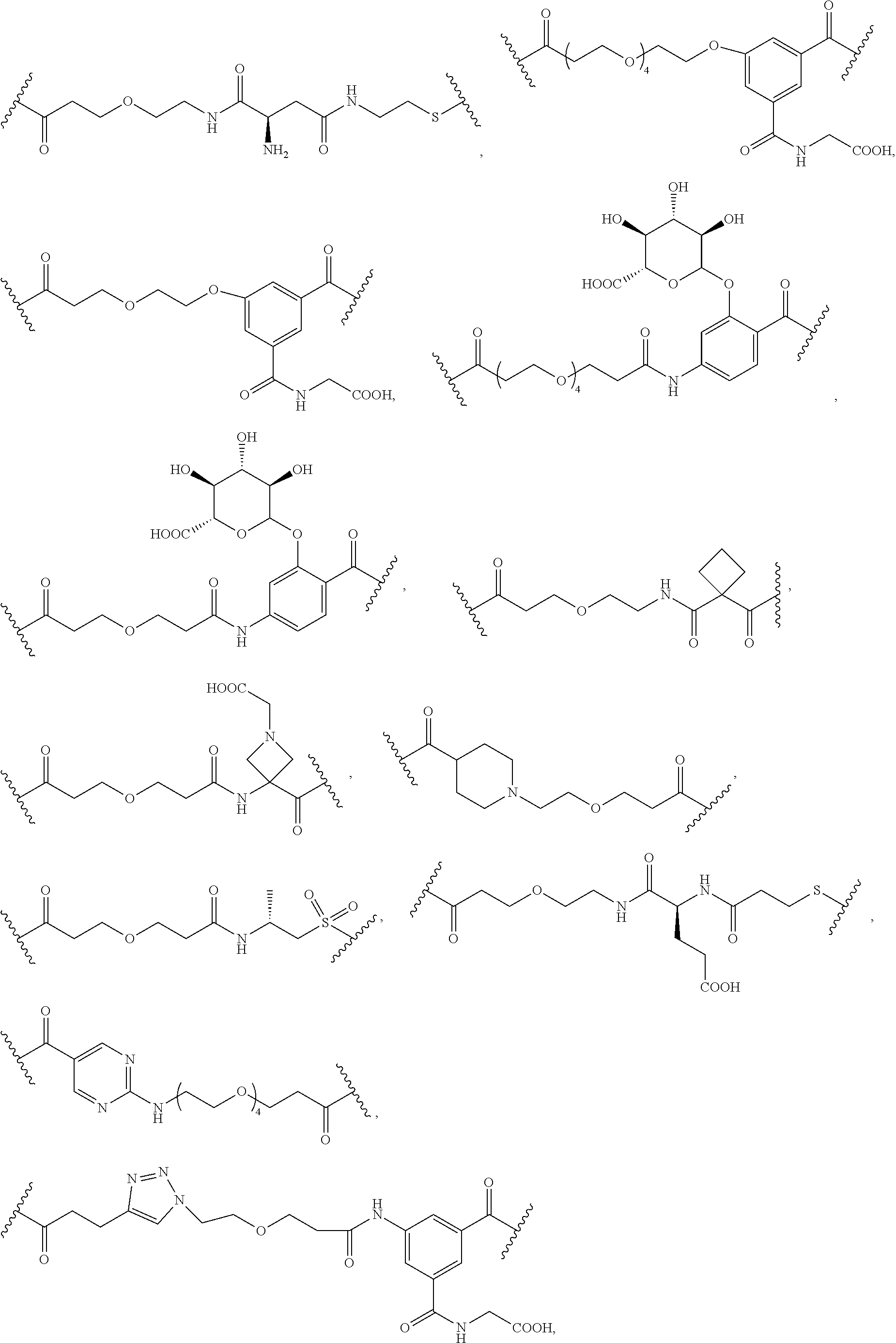

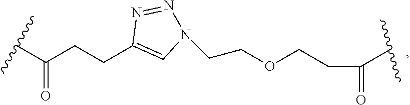 [SMILES: *C(=O)CCOCCn1cc(CCC(*)=O)nn1]

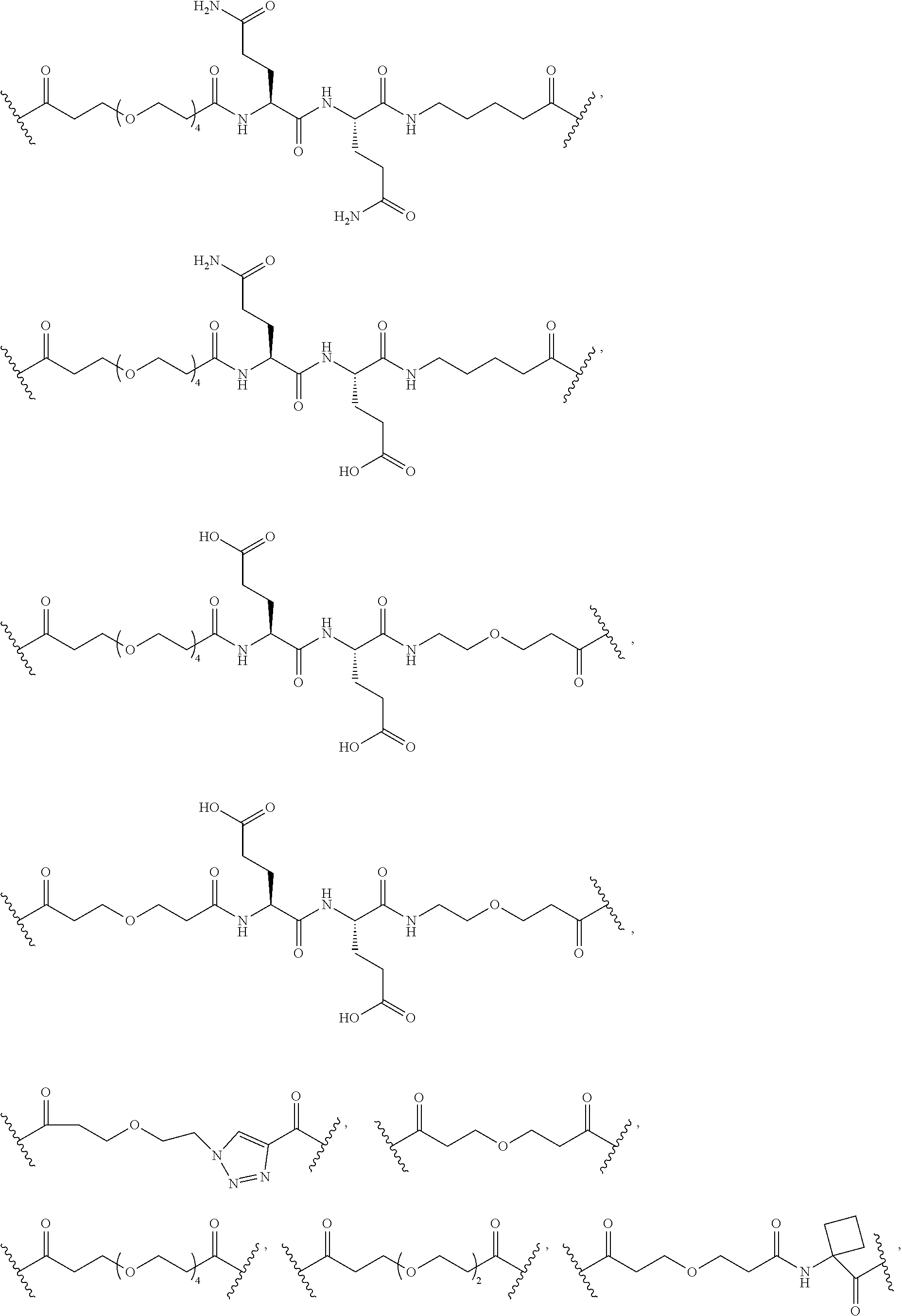

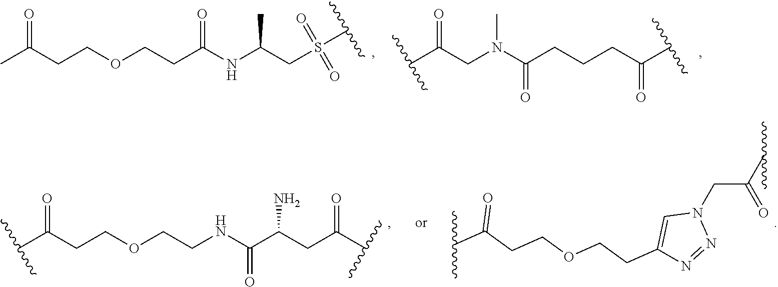 [SMILES: *S(=O)(=O)C[C@H](C)NC(=O)CCOCCC(C)=O ; *C(=O)CCCC(=O)N(C)CC(*)=O ; *C(=O)CCOCCNC(=O)[C@H](N)CC(*)=O ; *C(=O)CCOCCc1cn(CC(*)=O)nn1]

69. The compound of claim 55, or a pharmaceutically acceptable salt thereof, wherein the Spacer is or

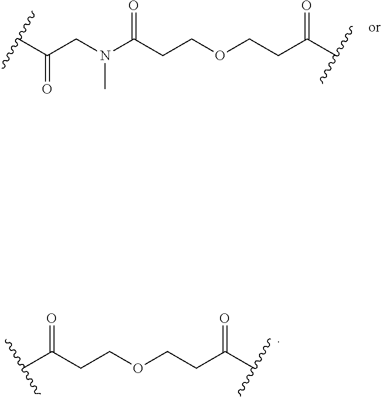 [SMILES: *C(=O)CCOCCC(=O)N(C)CC(*)=O ; *C(=O)CCOCCC(*)=O]

70. The compound of claim 55, or a pharmaceutically acceptable salt thereof, wherein the compound of Formula (I) is a compound of Formula (Ia):

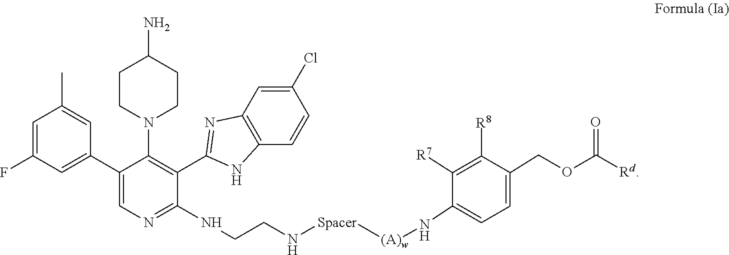

71. The compound of claim 55, or a pharmaceutically acceptable salt thereof, wherein R^{d}—H is monomethyl auristatin E.

72. A pharmaceutical composition comprising a compound of claim 55, or a pharmaceutically acceptable salt thereof, and a pharmaceutically acceptable excipient.

73. A method for the treatment of cancer comprising administering to a mammal with cancer an effective amount of a compound of claim 55, or a pharmaceutically acceptable salt thereof.

74. A compound selected from the group consisting of: Compounds 1-56, 59-62, 65, 67, 69, 71-78, 83, 86-91, 198-204, and 208-214, or a pharmaceutically acceptable salt thereof.

75. A compound selected from the group consisting of Compounds 205-207, 215-238, 246, and 247, or a pharmaceutically acceptable salt thereof.

76. A compound selected from the group consisting of Compounds 57, 58, 64, 66, 79-82, 84, 85, 92-197, 239-245, 248, and 249, or a pharmaceutically or a pharmaceutically acceptable salt thereof.

77. A compound that has the structure of Formula (II): 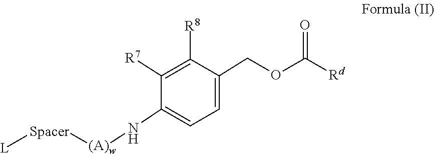
wherein:
L is a ligand;
Spacer is a spacer group;
each A is independently selected from natural or unnatural amino acids, wherein any free amine of an amino acid is optionally independently substituted with CH₃;
w is an integer selected from 1-6;
R⁷ is H, C₁-C₆ haloalkyl, OR^{7a}, 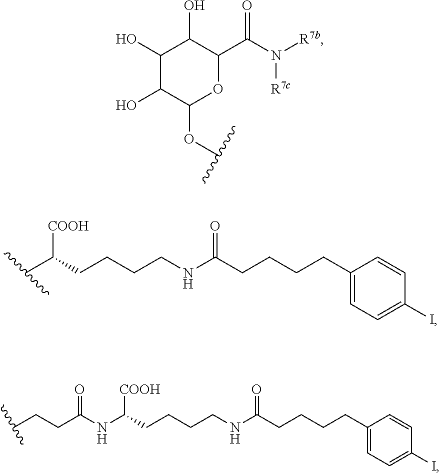
or C₁-C₆ alkyl optionally substituted with COOH;
R^{7a} is H, or C₁-C₆ alkyl optionally substituted with amino, COOH, or OH;
R^{7b} is H, C₁-C₂₀ alkyl optionally substituted with OR^{7b1}, COOR^{7b1}, NR^{7b1}₂, or SO₃R^{7b1}; (CH₂)₂NHC(═O)—R^{7b2}, 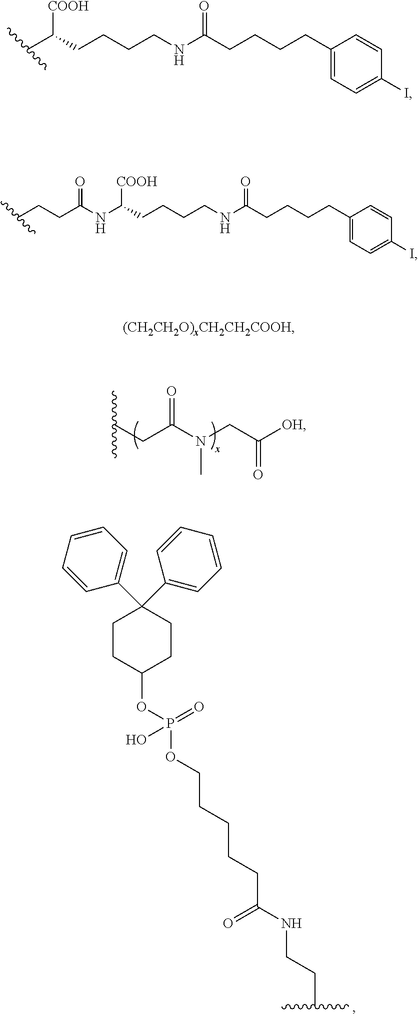 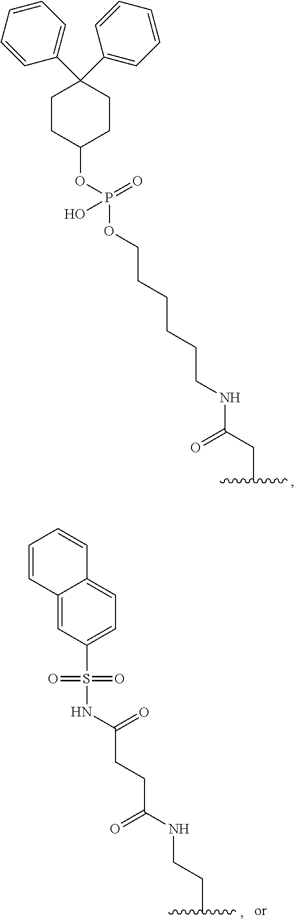 [SMILES: *CC(=O)NCCCCCCOP(=O)(O)OC1CCC(c2ccccc2)(c2ccccc2)CC1 ; *CCNC(=O)CCC(=O)NS(=O)(=O)c1ccc2ccccc2c1] 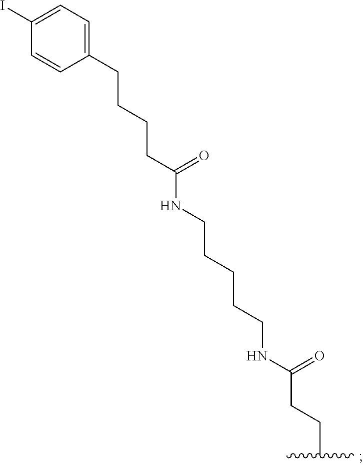 [SMILES: *CCC(=O)NCCCCCNC(=O)CCCCc1ccc(I)cc1]
each R^{7b1} is independently selected from H and C₁-C₆ alkyl;
R^{7b2} is (CH₂CH₂O)_{K}CH₂CH₂COOH, or (CH₂)_{y}COOH;
R^{7a} is H or C₁-C₆ alkyl; or
R^{7b} and R^{7c}, together with the nitrogen to which they are attached, combine to form a heterocycloalkyl group optionally substituted with C₁-C₆ alkyl which is optionally substituted with OH, COOH, or amino;
R⁸ is H, C₁-C₆ haloalkyl, COOH, C(═O)NH—R^{8a}, 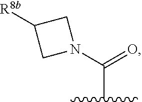
or C(═O)NH(CH₂)₂NHC(═O)—R^{8c};
R^{8a} is C₁-C₂₀ alkyl substituted with COOH or SO₃H, or C₁-C₆ alkoxy optionally substituted with amino;
R^{8b} is COOH or amino;
R^{8c} is (CH₂CH₂O)ₓCH₂CH₂COOH or (CH₂)_{y}COOH;
each x is independently an integer selected from 1-10;
each y is independently an integer selected from 1-20;
R^{d} is the residue of a bioactive molecule of the formula R^{d}—H;
wherein when R⁷ is H, then R⁸ is C₁-C₆ haloalkyl, COOH, C(═O)NH—R^{8a}, 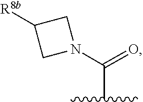
or C(═O)NH(CH₂)₂NHC(═O)—R^{8c}; and
wherein when R⁸ is H, then R⁷ is C₁-C₆ haloalkyl, OR^{7a}, 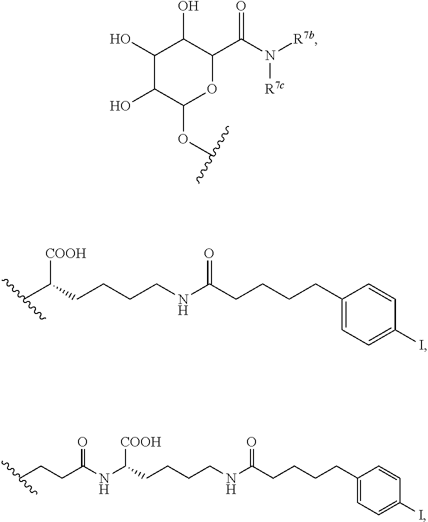
or C₁-C₆ alkyl optionally substituted with COOH.

78. The compound of claim 77, or a pharmaceutically acceptable salt thereof, wherein:
L is a ligand;
Spacer is a spacer group;
each A is independently selected from natural or unnatural amino acids, wherein any free amine of an amino acid is optionally independently substituted with CH₃;
w is an integer selected from 1-6;
R⁷ is H, C₁-C₆ alkyl, C₁-C₆ haloalkyl, OR^{7a}, or 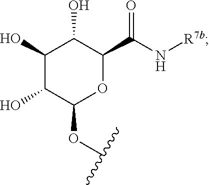
R^{7a} is H, or C₁-C₆ alkyl optionally substituted with amino, COOH, or OH;
R^{7b} is H, C₁-C₂₀ alkyl substituted with COOR^{7b1}, amino, or SO₃H, (CH₂)₂NHC(═O)—R^{7b2}, 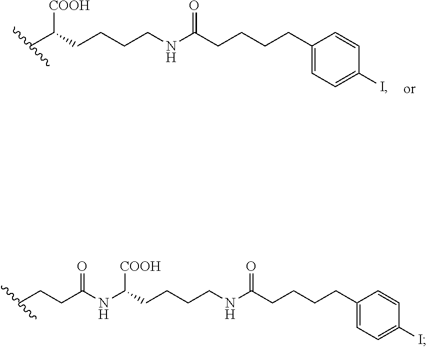
R^{7b1} is H or C₁-C₆ alkyl;
R^{7b2} is (CH₂CH₂O)ₓCH₂CH₂COOH, or (CH₂)_{y}COOH;
R⁸ is H, C₁-C₆ haloalkyl, COOH, C(═O)NH—R^{8a}, 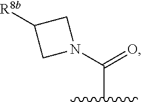
or C(═O)NH(CH₂)₂NHC(═O)—R^{8c};
R^{8s} is C₁-C₂₀ alkyl substituted with COOH or SO₃H, or C₁-C₆ alkoxy optionally substituted with amino;
R^{8b} is COOH or amino;
R^{8c} is (CH₂CH₂O)ₓCH₂CH₂COOH or (CH₂)_{y}COOH;
each x is independently an integer selected from 1-10;
each y is independently an integer selected from 1-20;
R^{d} is the residue of a bioactive molecule of the formula R^{d}—H;
wherein when R⁷ is H, then R⁸ is C₁-C₆ haloalkyl, COOH, C(═O)NH—R^{8a}, 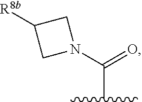
or C(═O)NH(CH₂)₂NHC(═O)—R^{8c}; and
wherein when R⁸ is H, then R⁷ is C₁-C₆ alkyl, C₁-C₆ haloalkyl, OR^{7a}, or

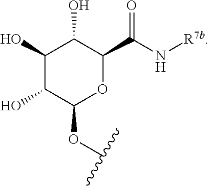

79. The compound of claim 77, or a pharmaceutically acceptable salt thereof, wherein R⁷ is

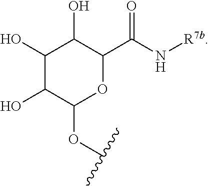

80. The compound of claim 77, or a pharmaceutically acceptable salt thereof, wherein R⁷ is OR^{7a} or

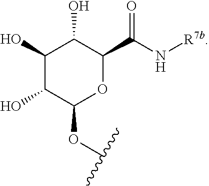

81. The compound of claim 77, or a pharmaceutically acceptable salt thereof, wherein 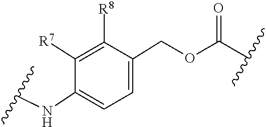
of Formula (II) is: 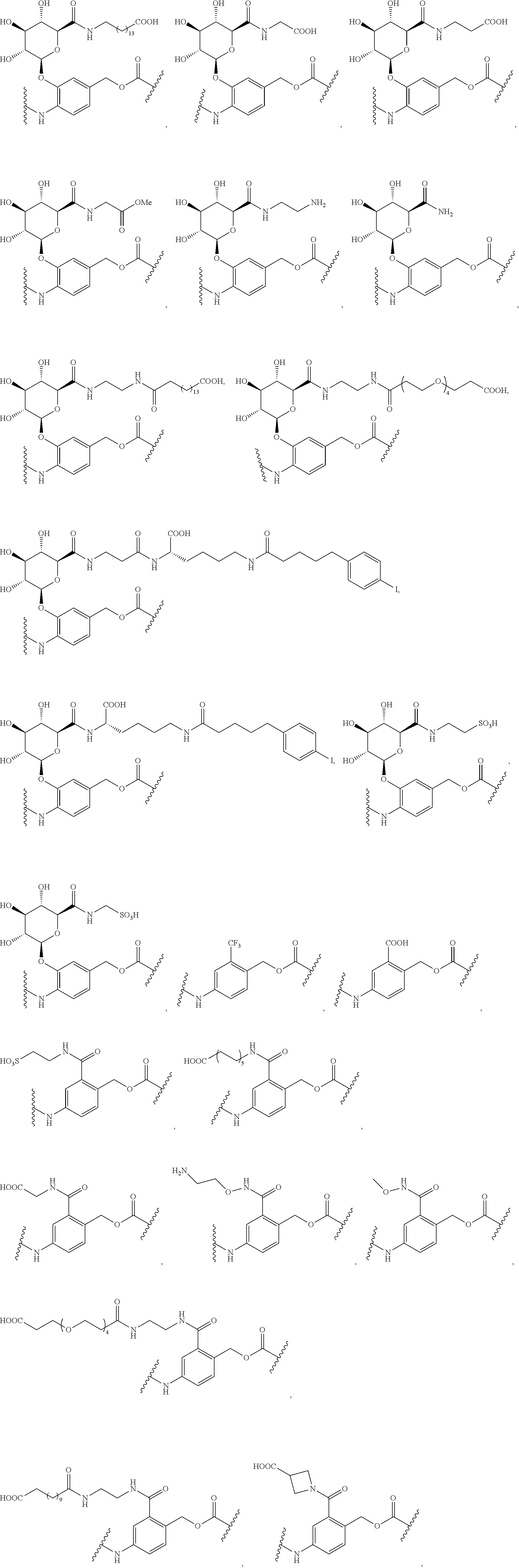 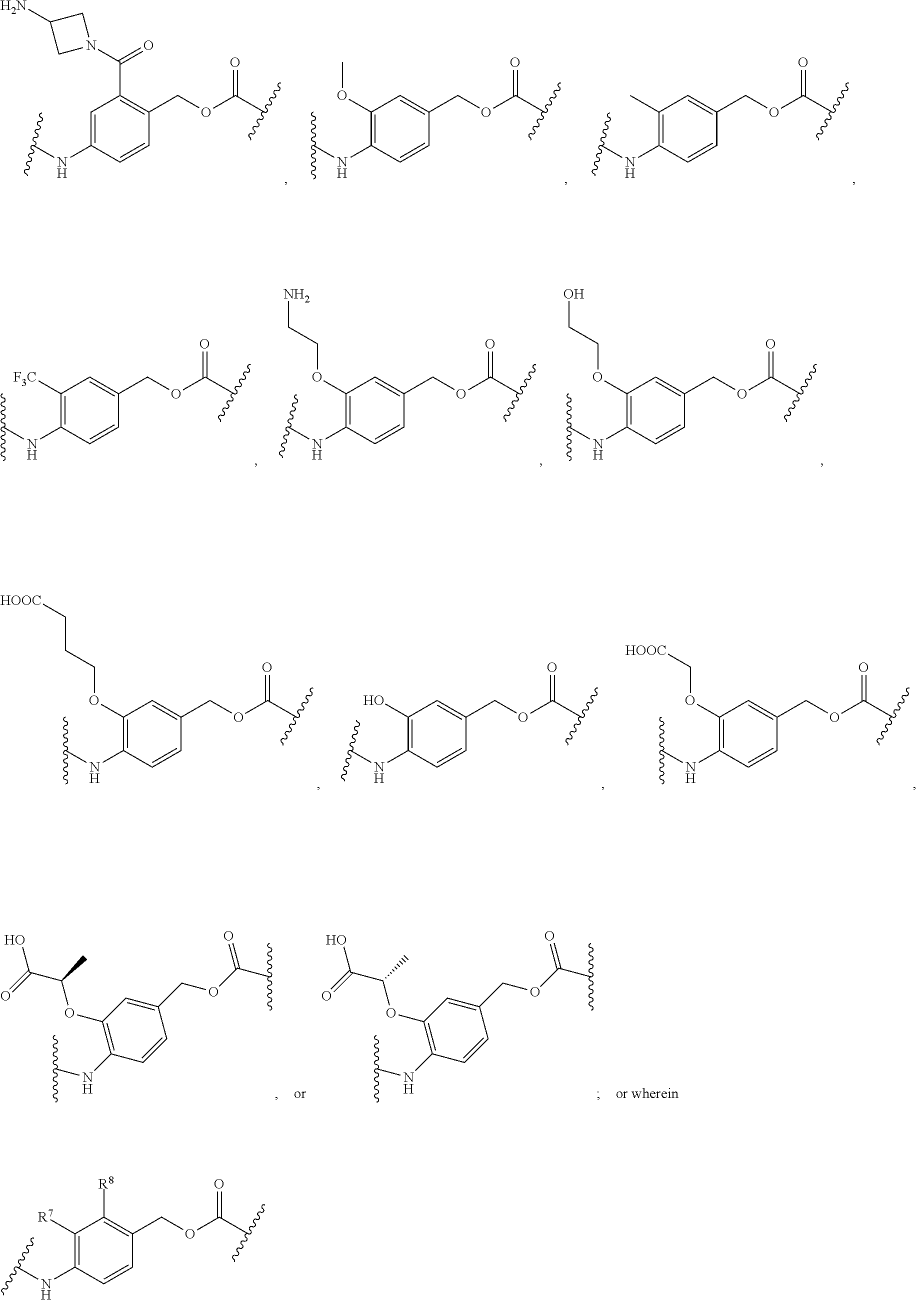
of Formula (II) is:

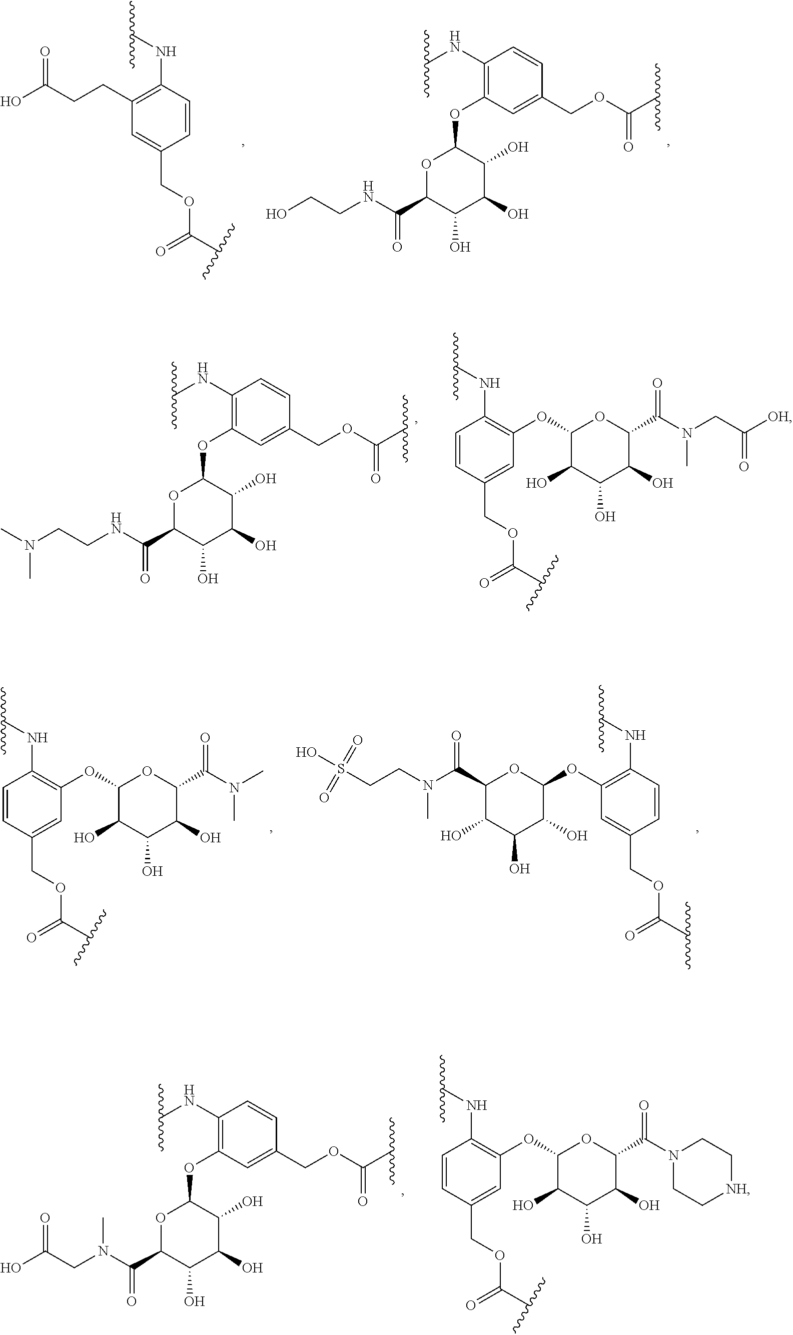

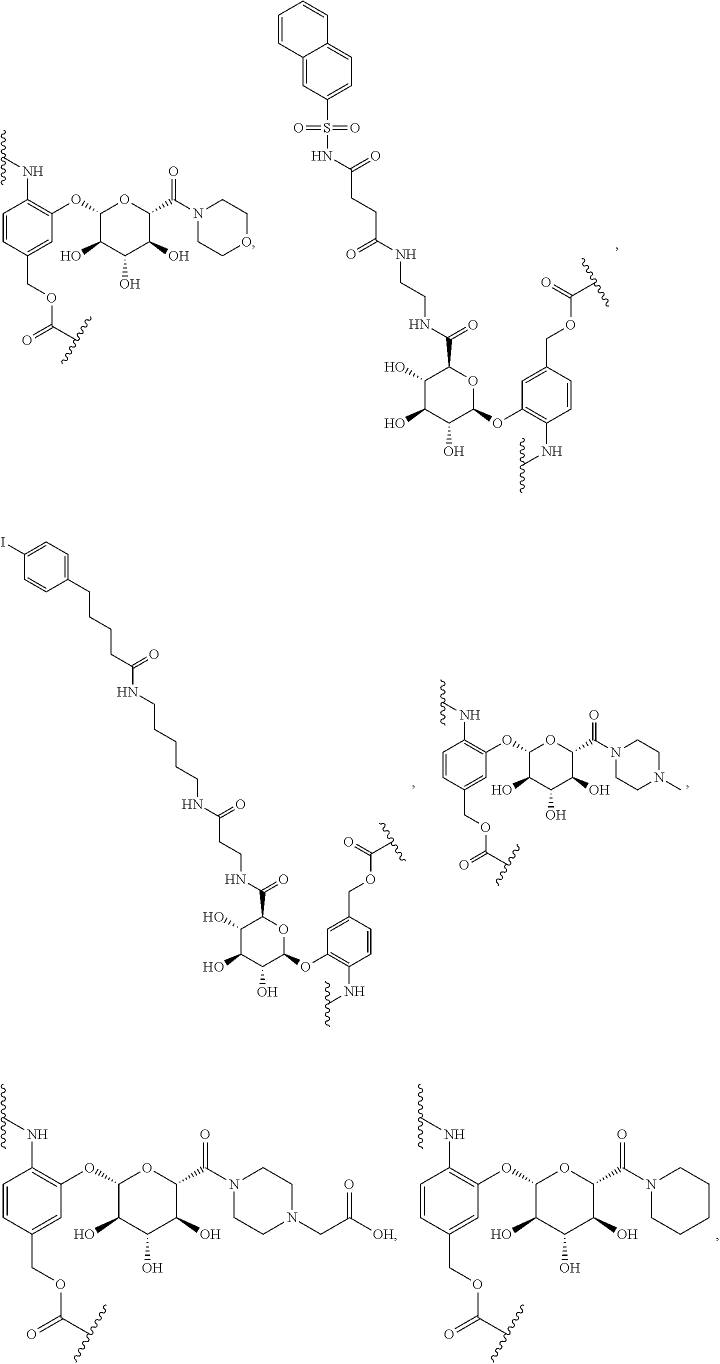

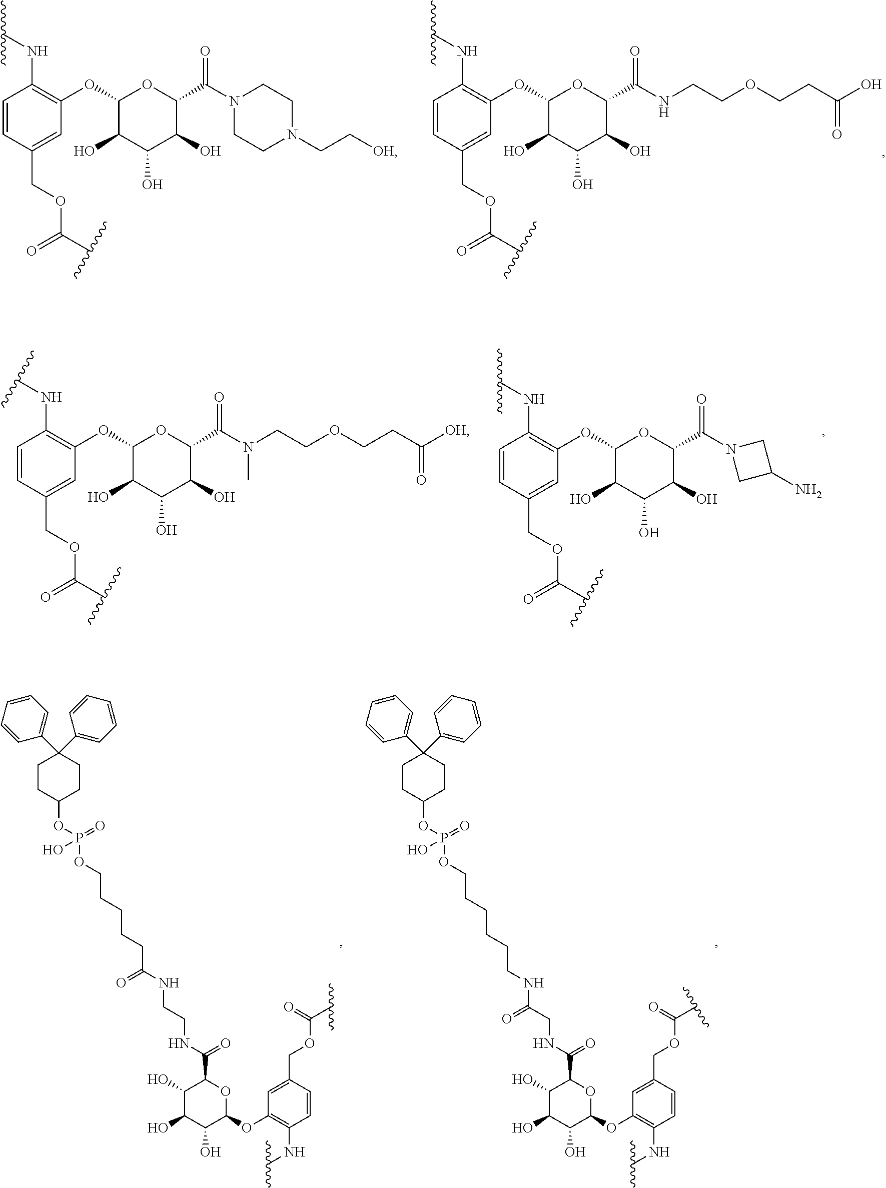

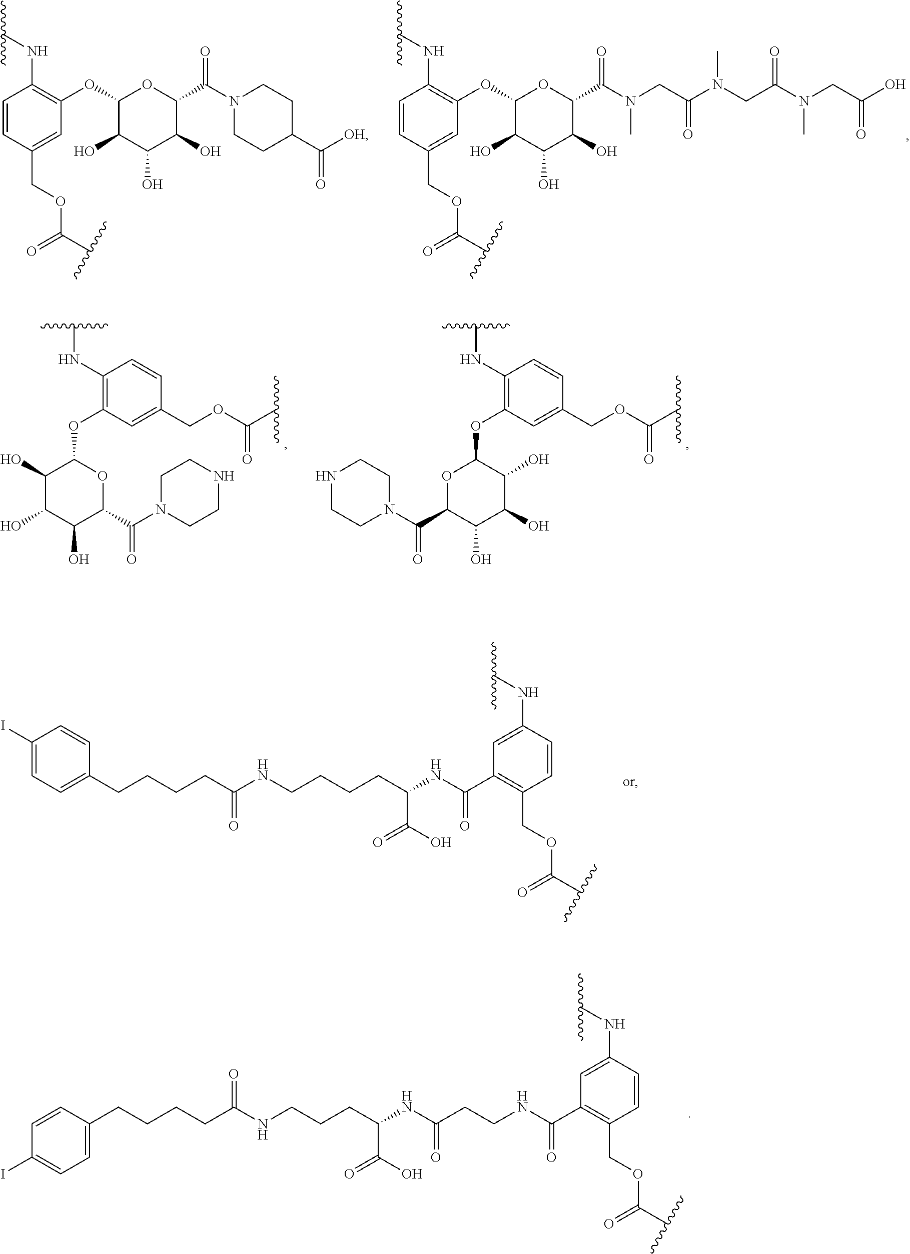 [SMILES: *Nc1ccc(COC(*)=O)cc1O[C@@H]1O[C@H](C(=O)N2CCC(C(=O)O)CC2)[C@@H](O)[C@H](O)[C@H]1O ; *Nc1ccc(COC(*)=O)cc1O[C@@H]1O[C@H](C(=O)N(C)CC(=O)N(C)CC(=O)N(C)CC(=O)O)[C@@H](O)[C@H](O)[C@H]1O ; *Nc1ccc(COC(*)=O)cc1O[C@@H]1O[C@H](C(=O)N2CCNCC2)[C@@H](O)[C@H](O)[C@H]1O ; *Nc1ccc(COC(*)=O)cc1O[C@@H]1O[C@H](C(=O)N2CCNCC2)[C@@H](O)[C@H](O)[C@H]1O ; *Nc1ccc(COC(*)=O)c(C(=O)N[C@@H](CCCCNC(=O)CCCCc2ccc(I)cc2)C(=O)O)c1 ; *Nc1ccc(COC(*)=O)c(C(=O)NCCC(=O)N[C@@H](CCCNC(=O)CCCCc2ccc(I)cc2)C(=O)O)c1]

82. The compound of claim 77, or a pharmaceutically acceptable salt thereof, wherein 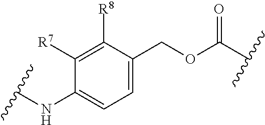
of Formula (II) is:

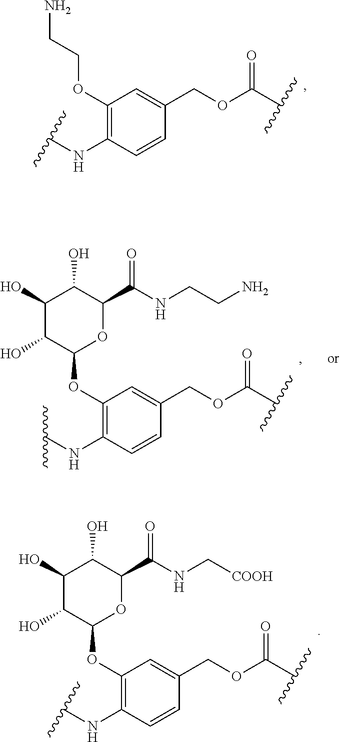 [SMILES: *Nc1ccc(COC(*)=O)cc1OCCN ; *Nc1ccc(COC(*)=O)cc1O[C@@H]1O[C@H](C(=O)NCCN)[C@@H](O)[C@H](O)[C@H]1O ; *Nc1ccc(COC(*)=O)cc1O[C@@H]1O[C@H](C(=O)NCC(=O)O)[C@@H](O)[C@H](O)[C@H]1O]

83. The compound of claim 77, or a pharmaceutically acceptable salt thereof, wherein each A is independently selected from natural or unnatural amino acids, wherein any free amine of an amino acid is optionally independently substituted with CH₃, wherein the natural or unnatural amino acids are selected from alanine (Ala), Ala(SO₃H), 3-(1-piperidinyl)alanine, cyclohexylalanine, arginine (Arg), asparagine (Asn), aspartate (Asp), cysteine (Cys), glutamine (Gin), glutamatic acid (Glu), glycine (Gly), leucine (Leu), lysine(Lys), methionine(Met), phenylalanine (Phe), homophenylalanine, proline (Pro), serine (Ser), 3-homoserine, tyrosine (Tyr), Tyr(SO₃H), tryptophan (Trp), valine (Val), citrulline, β-alanine, β3-homoserine, β3-homolysine, and β3-homoglutamic acid.
